(19) United States
(12) Patent Application Publication
(10) Pub. No.: US 2026/0167647 A1
(43) Pub. Date: Jun. 18, 2026

(54) THERAPEUTIC AGENTS

(71) Applicant: Charm Therapeutics Ltd., London (GB)

(72) Inventors: Andrew Robert Bayly, Sandhoe (GB); James David Cuthbertson, Cambridgeshire (GB); Sebastien Louis Degorce, Uttlesford (GB); Adam Ieuan Green, Greater Manchester (GB); Hannah Elspeth Bruce Macdonald, Central Bedfordshire (GB); Mark Pickworth, St. Albans (GB); Francesco Rianjongdee, London (GB)

(21) Appl. No.: 19/422,182

(22) Filed: Dec. 16, 2025

Related U.S. Application Data
(60) Provisional application No. 63/734,668, filed on Dec. 16, 2024.
(60) Provisional application No. 63/827,696, filed on Jun. 20, 2025.

(51) Int. Cl. C07D 487/08; A61K 31/551; A61P 35/02; C07D 519/00

(52) U.S. Cl. CPC C07D 487/08; A61K 31/551; A61P 35/02; C07D 519/00

(57) ABSTRACT

This disclosure provides chemical entities (e.g., a compound, or a pharmaceutically acceptable salt thereof, or an atropisomer thereof; or a pharmaceutically acceptable salt of an atropisomer) that modulate (e.g., inhibit) the interaction between menin and mixed-lineage leukaemia (“MLL”) proteins (e.g., MLL fusion proteins). The chemical entities are useful, e.g., for treating a subject (e.g., a human) having a condition, disease or disorder in which aberrant (e.g., increased, e.g., excessive) menin-MLL interaction contribute to the pathology, symptoms and/or progression of the condition, disease or disorder (e.g., cancer, diabetes). This disclosure also provides compositions containing the chemical entities as well as methods of making and using the same . . . .

### CROSS REFERENCE TO RELATED APPLICATIONS

This application claims the benefit of U.S. Provisional Application No. 63/734,668, filed on Dec. 16, 2024 and U.S. Provisional Application No. 63/827,696, filed on Jun. 20, 2025, each of which is incorporated herein by reference in its entirety.

### TECHNICAL FIELD

This disclosure provides chemical entities (e.g., a compound, or a pharmaceutically acceptable salt thereof, or an atropisomer thereof; or a pharmaceutically acceptable salt of an atropisomer) that modulate (e.g., inhibit) the interaction between menin and mixed-lineage leukaemia (“MLL”) proteins (e.g., MLL fusion proteins). The chemical entities are useful, e.g., for treating a subject (e.g., a human) having a condition, disease or disorder in which aberrant (e.g., increased, e.g., excessive) menin-MLL interaction contribute to the pathology, symptoms and/or progression of the condition, disease or disorder (e.g., cancer, diabetes). This disclosure also provides compositions containing the chemical entities as well as methods of making and using the same.

### BACKGROUND

The Histone-lysine N-methyltransferase 2 (KMT2) family of proteins are known to play an important role in the regulation of gene expression during early development and hematopoiesis (Rao & Dou, Nat. Rev. Cancer 15, 334-346 (2015)).

The human KMT2 family was initially named the mixed-lineage leukaemia (MLL) family, in part, because of the causal relationship between family member KMT2A and the onset of this particular form of leukaemia. KMT2A is commonly referred to as MLL (or MLL1), The menin protein, encoded by the MEN1 gene, is a scaffold protein that regulates gene expression by binding to the mixed lineage leukaemia methyltransferase, KMT2A (MLL; MLL1). It functions as both a tumour suppressor in mouse models of epithelial cancers and as an oncogene in leukaemias.

Menin is emerging as a key regulator of acute myeloid leukaemia (AML). Chimeric rearrangements in the mixed lineage leukaemia (MLL; MLL1; KMT2A) gene are found in 10% of all leukaemias including acute myeloid leukaemia (AML), acute lymphoblastic leukaemia (ALL) and mixed-lineage leukaemia (MLL). Mutations in the NPM1 gene are responsible for 30% of all acute myeloid leukaemias. NPM1 is primarily located in the nucleus, however mutations lead to cytoplasmic relocalisation (Falini et al., Blood. 2007 109 (3): 874-885). These two genetic abnormalities promote tumorigenesis when menin interacts with KMT2A, driving transcriptional activation of MEIS1 and HOX genes whose expression is a characteristic of AML. Activation of these genes keeps the aberrant cells in a state of pluripotency, blocking terminal differentiation and cell death (Grembecka et al., Nat. Chem. Biol. 2012; 8:277-284; Uckelmann et al., Science. 2020; 367:586-590; Krivtsov et al., Cancer Cell. 2019; 36:660-673). Therefore, disruption of the interaction between KMT2A and menin has been the focus of multiple drug discovery efforts.

Recent publications and clinical trials have shown that whilst first generation menin inhibitors can induce profound complete responses in relapsed or refractory AML with KMT2A rearrangements or NPM1 mutations, multiple different resistance mutations in the binding pocket of menin are now being detected in patients, including—but not limited to—M327I, M327V, G331R, T349M, S160C and S160T, rendering them less sensitive to further treatment.

### SUMMARY

This disclosure provides chemical entities (e.g., a compound, or a pharmaceutically acceptable salt thereof, or an atropisomer thereof; or a pharmaceutically acceptable salt of an atropisomer) that modulate (e.g., inhibit) the interaction between menin and mixed-lineage leukaemia (“MLL”) proteins (e.g., MLL fusion proteins). The chemical entities are useful, e.g., for treating a subject (e.g., a human) having a condition, disease or disorder in which aberrant (e.g., increased, e.g., excessive) menin-MLL interaction contribute to the pathology, symptoms and/or progression of the condition, disease or disorder (e.g., cancer, diabetes). This disclosure also provides compositions containing the chemical entities as well as methods of making and using the same. Advantageously, and without wishing to be bound by theory, it is believed that the chemical entities described herein can achieve equipotency and efficacy in the presence of both WT and mutant menin.

In one aspect, this disclosure provides compounds of Formula (I):

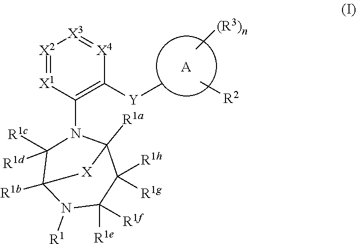

or a pharmaceutically acceptable salt thereof, or an atropisomer thereof, or a pharmaceutically acceptable salt of an atropisomer thereof, wherein:
R¹ is H, a nitrogen protecting group, or

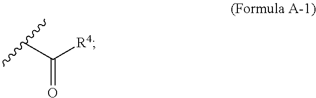

X is CH₂, CH(CH₃), or C(CH₃)₂;
each of R^{1a} and R^{1b} is independently selected from the group consisting of H and CH₃;
each of R^{1c}, R^{1d}, R^{1e}, R^{1f}, R^{1g}, and R^{1h} is independently selected from the group consisting of H, halo, C₁-C₂ alkyl, and C₁-C₂ haloalkyl;
one, two, or three of X¹, X², X³, and X⁴ are N, and the other(s) are an independently selected CR⁵,
Y is O, S, S(O), or SO₂;
Ring A is:
   C₆₋₁₀ aryl; or
   heteroaryl of 5-10 ring atoms, wherein 1-4 ring atoms are heteroatoms, each independently selected from the group consisting of N, N(H), N(R^{d}), O, and S(O)₀₋₂;
R² is selected from the group consisting of

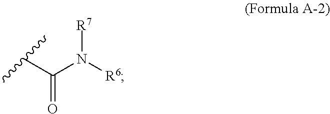

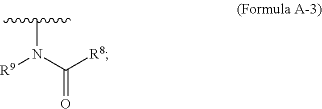

### and R¹⁰;

each occurrence of R³ is, independently, selected from the group consisting of: halo; C₁₋₄ alkyl; C₂₋₄ alkynyl; C₁₋₄ alkoxy; C₁₋₄ haloalkoxy; —S(O)₁₋₂(C₁₋₄ alkyl); and cyano;
n is 0, 1 or 2;
R⁴ is:
   C₁₋₈ alkyl or C₂₋₈ alkenyl, each of which is optionally substituted with 1-3 independently selected R^{a},
   L₁-C₃₋₁₀ cycloalkyl or C₃₋₁₀ cycloalkenyl, each of which is optionally substituted with 1-4 substituents independently selected from the group consisting of oxo, R^{b}, and R^{g};
   L₁-heterocyclyl or L₁-heterocycloalkenyl of 3-12 ring atoms, wherein 1-3 ring atoms are heteroatoms, each independently selected from the group consisting of N, N(H), N(O—), N(R^{d}), O, and S(O)₀₋₂, and wherein the heterocyclyl or heterocycloalkenyl is optionally substituted with 1-4 substituents independently selected from the group consisting of oxo, R^{b}, and R^{g};
   L₁-heteroaryl of 5-12 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of oxo, R^{c}, and R^{g}; or
   L₁-C₆₋₁₀ aryl optionally substituted with 1-4 substituents independently selected from the group consisting of oxo, R^{c}, and R^{g};
L₁ is a bond or C₁₋₄ alkylene;
R⁵ is H, C₁₋₂ alkoxy, or C₁₋₂ alkyl;
each of R⁶ and R⁷ is independently selected from the group consisting of;
   H;
   C₁₋₆ alkyl optionally substituted with 1-4 independently selected R^{a}; and
   C₃₋₈ cycloalkyl optionally substituted with substituents independently selected from the group consisting of oxo, R^{b}, and R^{g}; or
R⁶ and R⁷ together with the nitrogen atom to which each is attached forms a heterocyclyl of 3-8 ring atoms, wherein 1-3 ring atoms are heteroatoms, each independently selected from the group consisting of N, N(H), N(R^{d}), O, and S(O)₀₋₂, and wherein the heterocyclyl is optionally substituted with 1-4 substituents independently selected from the group consisting of oxo, R^{b}, and R^{g};
each of R¹ and R⁹ is independently selected from the group consisting of;
   H;
   C₁₋₆ alkyl optionally substituted with 1-4 independently selected R^{a}; and
   C₃₋₈ cycloalkyl optionally substituted with substituents independently selected from the group consisting of oxo, R^{b}, and R^{g};
R¹⁰ is:
   L₁-heteroaryl of 5-12 ring atoms or heterocyclyl of 5-12 ring atoms, wherein 1-4 ring atoms are heteroatoms, each independently selected from the group consisting of N, N(H), N(R^{d}), O, and S(O)₀₋₂, and wherein each of which is optionally substituted with 1-4 substituents independently selected from the group consisting of oxo, R^{c}, and R^{g};
   L₁-C₆₋₁₀ aryl optionally substituted with 1-4 substituents independently selected from the group consisting of oxo, R^{c}, and R^{g};
   C₁₋₆ alkoxy;
   C₃₋₈ cycloalkyl optionally substituted with substituents independently selected from the group consisting of oxo, R^{b}, and R^{g};
each occurrence of R^{a} is independently selected from the group consisting of —OH; -halo; —NR^{e}R^{f}; C₁₋₄ alkoxy; C₁₋₄ haloalkoxy; —C(═O)O(C₁₋₄ alkyl); —C(═O)(C₁₋₄ alkyl); —OC(═O)(C₁₋₄ alkyl); —C(═O)OH; —CONR′R″; —S(O)₁₋₂NR′R″; —S(O)₁₋₂(C₁₋₄ alkyl); and cyano;
each occurrence of R^{b} and R^{c} is independently selected from the group consisting of halo; cyano; C₁₋₁₀ alkyl which is optionally substituted with 1-6 independently selected R^{a}; C₂₋₆ alkenyl; C₂₋₆ alkynyl; C₁₋₄ alkoxy; C₁₋₄ haloalkoxy; —S(O)₁₋₂(C₁₋₄ alkyl); —S(O)(═NH)(C₁₋₄ alkyl); —NR^{e}R^{f}; —OH; —S(O)₁₋₂NR′R″; —C₁₋₄ thioalkoxy; —NO₂; —C(═O)(C₁₋₁₀ alkyl); —C(═O)O(C₁₋₄ alkyl); —OC(═O)(C₁₋₄ alkyl); —C(═O)OH; —C(═O)NR′R″; —NR′C(═O)(C₁₋₄ alkyl) and —SF₅;
each occurrence of R^{d} is independently selected from the group consisting of: C₁₋₆ alkyl optionally substituted with 1-3 independently selected R^{a}; —C(O)(C₁₋₄ alkyl); —C(O)O(C₁₋₄ alkyl); —CONR′R″; —S(O)₁₋₂NR′R″; —S(O)₁₋₂(C₁₋₄ alkyl); —OH; and C₁₋₄ alkoxy;
each occurrence of R^{e} and R^{f} is independently selected from the group consisting of: H; C₁₋₆ alkyl optionally substituted with 1-3 substituents each independently selected from the group consisting of NR′R″, —OH, halo, C₁₋₄ alkoxy, and C₁₋₄ haloalkoxy; —C(O)(C₁₋₄ alkyl); —C(O)O(C₁₋₄ alkyl); —CONR′R″; —S(O)₁₋₂NR′R″; —S(O)₁₋₂(C₁₋₄ alkyl); —OH; and C₁₋₄ alkoxy;
each occurrence of R⁹ is independently selected from the group consisting of:
   C₃₋₁₂ cycloalkyl or C₃₋₁₂ cycloalkenyl, each of which is optionally substituted with 1-4 substituents independently selected from the group consisting of oxo, R^{b}, and R^{h};
   heterocyclyl or heterocycloalkenyl of 3-12 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo, R^{b}, and R^{h};
   heteroaryl of 5-12 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of oxo, R^{e}, and R^{h}; and
   C₆₋₁₀ aryl optionally substituted with 1-4 substituents independently selected from the group consisting of oxo, R^{e}, and R^{h};
each occurrence of R^{h} is independently selected from the group consisting of
   C₃₋₁₂ cycloalkyl or C₃₋₁₂ cycloalkenyl, each of which is optionally substituted with 1-4 Rⁱ;
   heterocyclyl or heterocycloalkenyl of 3-12 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 Rⁱ;
   heteroaryl of 5-12 ring atoms, wherein 1-3 ring atoms are heteroatoms, each independently selected from the group consisting of N, N(H), N(R^{d}), O, and S(O)₀₋₂, and wherein the heteroaryl is optionally substituted with 1-4 Rⁱ; and
   C₆₋₁₀ aryl optionally substituted with 1-4 Rⁱ;
each occurrence of Rᵢ is independently selected from the group consisting of C₁₋₆ alkyl; C₁₋₄ haloalkyl; C₁₋₄ alkoxy; C₁₋₄ haloalkoxy; and halo; and each occurrence of R′ and R″ is independently selected from the group consisting of H; —OH; and C₁₋₄ alkyl.

In another aspect, this disclosure provides compounds of Formula (I):

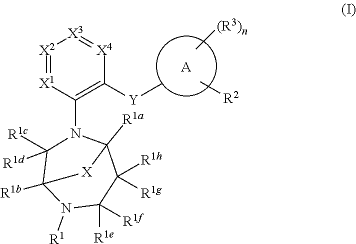

or a pharmaceutically acceptable salt thereof, or an atropisomer thereof, of a pharmaceutically acceptable salt of an atropisomer thereof, wherein:
R¹ is H, a nitrogen protecting group, or

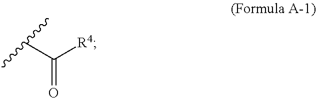

X is CH₂, CH(CH₃), or C(CH₃)₂;
each of R^{1a} and R^{1b} is independently selected from the group consisting of H and CH₃;
each of R^{1c}, R^{1d}, R^{1e}, R^{1f}, R^{1g}, and R^{1h} is independently selected from the group consisting of H, halo, C₁-C₂ alkyl, and C₁-C₂ haloalkyl;
one, two, or three of X¹, X², X³, and X⁴ are N, and the other(s) are an independently selected CR⁵,
Y is O, S, S(O), or SO₂;
Ring A is:
   C₆₋₁₀ aryl; or
   heteroaryl of 5-10 ring atoms, wherein 1-4 ring atoms are heteroatoms, each independently selected from the group consisting of N, N(H), N(R^{d}), O, and S(O)₀₋₂;
R² is:

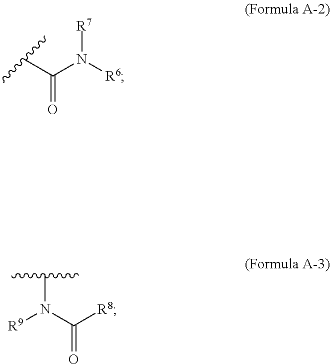

or R¹⁰;
each occurrence of R³ is, independently, selected from the group consisting of: halo; C₁₋₄ alkyl; C₂₋₄ alkynylC₁₋₄ alkoxy; C₁₋₄ haloalkoxy; —S(O)₁₋₂(C₁₋₄ alkyl); and cyano;
n is 0, 1 or 2;
R⁴ is:
   C₁₋₈ alkyl or C₂₋₈ alkenyl, each of which is optionally substituted with 1-3 independently selected R^{a},
   L₁-C₃₋₁₀ cycloalkyl or L₁-C₃₋₁₀ cycloalkenyl, each of which is optionally substituted with 1-4 substituents independently selected from the group consisting of oxo, R^{b}, and R^{g};
   L₁-heterocyclyl or L₁-heterocycloalkenyl of 3-12 ring atoms, wherein 1-3 ring atoms are heteroatoms, each independently selected from the group consisting of N, N(H), N(R^{d}), N(O⁻), O, and S(O)₀₋₂, and wherein the heterocyclyl or heterocycloalkenyl is optionally substituted with 1-4 substituents independently selected from the group consisting of oxo, R^{b}, and R^{g};
   L₁-heteroaryl of 5-12 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of oxo, R^{c}, and R^{g}; or
L₁-C₆₋₁₀ aryl optionally substituted with 1-4 substituents independently selected from the group consisting of oxo, R^{c}, and R^{g};
L₁ is a bond or C₁₋₄ alkylene;
R⁵ is H, C₁₋₂ alkoxy or C₁₋₂ alkyl;
each of R⁶ and R⁷ is independently selected from the group consisting of,
   H;
   C₁₋₆ alkyl optionally substituted with 1-4 independently selected R^{a}; and
   C₃₋₈ cycloalkyl optionally substituted with substituents independently selected from the group consisting of oxo, R^{b}, and R^{g}; or
R⁶ and R⁷ together with the nitrogen atom to which each is attached forms a heterocyclyl of 3-8 ring atoms, wherein 1-3 ring atoms are heteroatoms, each independently selected from the group consisting of N, N(H), N(R^{d}), O, and S(O)₀₋₂, and wherein the heterocyclyl is optionally substituted with 1-4 substituents independently selected from the group consisting of oxo, R^{b}, and R^{g};
each of R⁸ and R⁹ is independently selected from the group consisting of;
   H;
   C₁₋₆ alkyl optionally substituted with 1-4 independently selected R^{a}; and
   C₃₋₈ cycloalkyl optionally substituted with substituents independently selected from the group consisting of oxo, R^{b}, and R^{g};
R¹⁰ is:
   L₁-heteroaryl of 5-12 ring atoms or heterocyclyl of 5-12 ring atoms, wherein 1-4 ring atoms are heteroatoms, each independently selected from the group consisting of N, N(H), N(R^{d}), O, and S(O)₀₋₂, and wherein each is optionally substituted with 1-4 substituents independently selected from the group consisting of oxo, R^{c}, and R^{g};
   L₁-C₆₋₁₀ aryl optionally substituted with 1-4 substituents independently selected from the group consisting of oxo, R^{c}, and R^{g};
   C₁₋₆ alkoxy;
   C₃₋₈ cycloalkyl optionally substituted with substituents independently selected from the group consisting of oxo, R^{b}, and R^{g}; and
   L₁-heterocyclyl or L₁-heterocycloalkenyl of 3-12 ring atoms, wherein 1-3 ring atoms are heteroatoms, each independently selected from the group consisting of N, N(H), N(R^{d}), N(O—), O, and S(O)₀₋₂, and wherein the heterocyclyl or heterocycloalkenyl is optionally substituted with 1-4 substituents independently selected from the group consisting of oxo, R^{b}, and R^{g};
each occurrence of R^{a} is independently selected from the group consisting of —OH; -halo; —NR^{e}R^{f}; C₁₋₄ alkoxy; C₁₋₄ haloalkoxy; —C(═O)O(C₁₋₄ alkyl); —C(═O)(C₁₋₄ alkyl); —OC(═O)(C₁₋₄ alkyl); —C(═O)OH; —CONR′R″; —S(O)₁₋₂NR′R″; —S(O)₁₋₂(C₁₋₄ alkyl); and cyano;
each occurrence of R^{b} and R^{c} is independently selected from the group consisting of halo; cyano; C₁₋₁₀ alkyl which is optionally substituted with 1-6 independently selected R^{a}; C₂₋₆ alkenyl; C₂₋₆ alkynyl; C₁₋₄ alkoxy; C₁₋₄ haloalkoxy; —S(O)₁₋₂(C₁₋₄ alkyl); —S(O)(═NH)(C₁₋₄ alkyl); —NR^{e}R^{f}; —OH; —S(O)₁₋₂NR′R″; —C₁₋₄ thioalkoxy; —NO₂; —C(═O)(C₁₋₁₀ alkyl); —C(═O)O(C₁₋₄ alkyl); —OC(═O)(C₁₋₄ alkyl); —C(═O)OH; —C(═O)NR′R″; —NR′C(═O)(C₁₋₄ alkyl) and —SF₅;
each occurrence of R^{d} is independently selected from the group consisting of: C₁₋₆ alkyl optionally substituted with 1-3 independently selected R^{a}; —C(O)(C₁₋₄ alkyl); —C(O)O(C₁₋₄ alkyl); —CONR′R″; —S(O)₁₋₂NR′R″; —S(O)₁₋₂(C₁₋₄ alkyl); —OH; benzyl, and C₁₋₄ alkoxy;
each occurrence of R^{e} and R^{f} is independently selected from the group consisting of: H; C₁₋₆ alkyl optionally substituted with 1-3 substituents each independently selected from the group consisting of NR′R″, —OH, halo, C₁₋₄ alkoxy, and C₁₋₄ haloalkoxy; —C(O)(C₁₋₄ alkyl); —C(O)O(C₁₋₄ alkyl); —CONR′R″; —S(O)₁₋₂NR′R″; —S(O)₁₋₂(C₁₋₄ alkyl); —OH; and C₁₋₄ alkoxy;
each occurrence of R⁹ is independently selected from the group consisting of:
   C₃₋₁₂ cycloalkyl or C₃₋₁₂ cycloalkenyl, each of which is optionally substituted with 1-4 substituents independently selected from the group consisting of oxo, R^{b}, and R^{b};
   heterocyclyl or heterocycloalkenyl of 3-12 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo, R^{b}, and R^{b};
   heteroaryl of 5-12 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of oxo, R^{e}, and R^{h}; and
   C₆₋₁₀ aryl optionally substituted with 1-4 substituents independently selected from the group consisting of oxo, R^{e}, and R^{b};
each occurrence of R^{h} is independently selected from the group consisting of
   C₃₋₁₂ cycloalkyl or C₃₋₁₂ cycloalkenyl, each of which is optionally substituted with 1-4 Rⁱ;
   heterocyclyl or heterocycloalkenyl of 3-12 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 Rⁱ;
   heteroaryl of 5-12 ring atoms, wherein 1-3 ring atoms are heteroatoms, each independently selected from the group consisting of N, N(H), N(R^{d}), O, and S(O)₀₋₂, and wherein the heteroaryl is optionally substituted with 1-4 Rⁱ; and
   C₆₋₁₀ aryl optionally substituted with 1-4 Rⁱ;
each occurrence of Rⁱ is independently selected from the group consisting of C₁₋₆ alkyl; C₁₋₄ haloalkyl; C₁₋₄ alkoxy; C₁₋₄ haloalkoxy; and halo; and
each occurrence of R′ and R″ is independently selected from the group consisting of: H; —OH; and C₁₋₄ alkyl.

In a further aspect, this disclosure features compounds of formula (I):
(I):

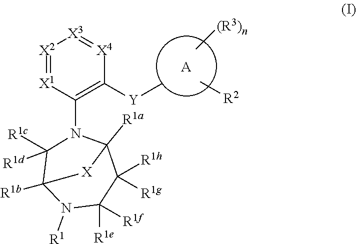

or a pharmaceutically acceptable salt thereof, or an atropisomer thereof, of a pharmaceutically acceptable salt of an atropisomer thereof, wherein:
R¹ is H, a nitrogen protecting group, or

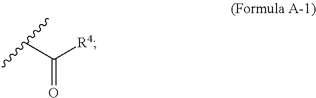

X is CH₂, CH(CH₃), or C(CH₃)₂;
each of R^{1a} and R^{1b} is independently selected from the group consisting of H and CH₃;
each of R^{1c}, R^{1d}, R^{1e}, R^{1f}, R^{1g}, and R^{1h} is independently selected from the group consisting of H, halo, C₁-C₂ alkyl, and C₁-C₂ haloalkyl;
one, two, or three of X¹, X², X³, and X⁴ are N, and the other(s) are an independently selected CR⁵,
Y is O, S, S(O), or SO₂;
Ring A is:
   C₆₋₁₀ aryl; or
   heteroaryl of 5-10 ring atoms, wherein 1-4 ring atoms are heteroatoms,
each independently selected from the group consisting of N, N(H), N(R^{d}), O, and S(O)₀₋₂;
R² is:

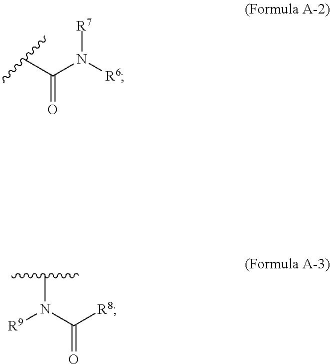

R¹⁰; —C₁-C₄ alkylene-NR^{e}R^{f}; or —OR^{g};
each occurrence of R³ is, independently, selected from the group consisting of: halo; C₁₋₄ alkyl; C₂₋₄ alkynylC₁₋₄ alkoxy; C₁₋₄ haloalkoxy; —S(O)₁₋₂(C₁₋₄ alkyl); and cyano;
n is 0, 1 or 2;
R⁴ is:
   C₁₋₈ alkyl or C₂₋₈ alkenyl, each of which is optionally substituted with 1-3 independently selected R^{a},
   L₁-C₃₋₁₀ cycloalkyl or L₁-C₃₋₁₀ cycloalkenyl, each of which is optionally substituted with 1-4 substituents independently selected from the group consisting of oxo, R^{b}, and R^{g};
   L₁-heterocyclyl or L₁-heterocycloalkenyl of 3-12 ring atoms, wherein 1-3 ring atoms are heteroatoms, each independently selected from the group consisting of N, N(H), N(R^{d}), N(O⁻), O, and S(O)₀₋₂, and wherein the heterocyclyl or heterocycloalkenyl is optionally substituted with 1-4 substituents independently selected from the group consisting of oxo, R^{b}, and R^{g};
   L₁-heteroaryl of 5-12 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of oxo, R^{c}, and R^{g}; or
   L₁-C₆₋₁₀ aryl optionally substituted with 1-4 substituents independently selected from the group consisting of oxo, R^{c}, and R^{g};
L₁ is a bond or C₁₋₄ alkylene;
R⁵ is H, C₁₋₂ alkoxy or C₁₋₂ alkyl;
each of R⁶ and R⁷ is independently selected from the group consisting of,
   H;
   C₁₋₆ alkyl optionally substituted with 1-4 independently selected R^{a}; and
   C₃₋₈ cycloalkyl optionally substituted with substituents independently selected from the group consisting of oxo, R^{b}, and R^{g}; or
R⁶ and R⁷ together with the nitrogen atom to which each is attached forms a heterocyclyl of 3-8 ring atoms, wherein 1-3 ring atoms are heteroatoms, each independently selected from the group consisting of N, N(H), N(R^{d}), O, and S(O)₀₋₂, and wherein the heterocyclyl is optionally substituted with 1-4 substituents independently selected from the group consisting of oxo, R^{b}, and R^{g};
each of R⁸ and R⁹ is independently selected from the group consisting of,
   H;
   C₁₋₆ alkyl optionally substituted with 1-4 independently selected R^{a}; and
   C₃₋₈ cycloalkyl optionally substituted with substituents independently selected from the group consisting of oxo, R^{b}, and R^{g};
R¹⁰ is:
   L₁-heteroaryl of 5-12 ring atoms or heterocyclyl of 5-12 ring atoms, wherein 1-4 ring atoms are heteroatoms, each independently selected from the group consisting of N, N(H), N(R^{d}), O, and S(O)₀₋₂, and wherein each is optionally substituted with 1-4 substituents independently selected from the group consisting of oxo, R^{c}, and R^{g};
   L₁-C₆₋₁₀ aryl optionally substituted with 1-4 substituents independently selected from the group consisting of oxo, R^{c}, and R^{g};
   C₁₋₆ alkoxy;
   C₃₋₈ cycloalkyl optionally substituted with substituents independently selected from the group consisting of oxo, R^{b}, and R^{g}; and
   L₁-heterocyclyl or L₁-heterocycloalkenyl of 3-12 ring atoms, wherein 1-3 ring atoms are heteroatoms, each independently selected from the group consisting of N, N(H), N(R^{d}), N(O⁻), O, and S(O)₀₋₂, and wherein the heterocyclyl or heterocycloalkenyl is optionally substituted with 1-4 substituents independently selected from the group consisting of oxo, R^{b}, and R^{g};
each occurrence of R^{a} is independently selected from the group consisting of —OH; -halo; —NR^{e}R^{f}; C₁₋₄ alkoxy; C₁₋₄ haloalkoxy; —C(═O)O(C₁₋₄ alkyl); —C(═O)(C₁₋₄ alkyl); —OC(═O)(C₁₋₄ alkyl); —C(═O)OH; —CONR′R″; —S(O)₁₋₂NR′R″; —S(O)₁₋₂(C₁₋₄ alkyl); and cyano;
each occurrence of R^{b} and R^{c} is independently selected from the group consisting of halo; cyano; C₁₋₁₀ alkyl which is optionally substituted with 1-6 independently selected R^{a}; C₂₋₆ alkenyl; C₂₋₆ alkynyl; C₁₋₄ alkoxy; C₁₋₄ haloalkoxy; —S(O)₁₋₂(C₁₋₄ alkyl); —S(O)(═NH)(C₁₋₄ alkyl); —NR^{e}R^{f}; —OH; —S(O)₁₋₂NR′R″; —C₁₋₄ thioalkoxy; —NO₂; —C(═O)(C₁₋₁₀ alkyl); —C(═O)O(C₁₋₄ alkyl); —OC(═O)(C₁₋₄ alkyl); —C(═O)OH; —C(═O)NR′R″; —NR′C(═O)(C₁₋₄ alkyl) and —SF₅;
each occurrence of R^{d} is independently selected from the group consisting of: C₁₋₆ alkyl optionally substituted with 1-3 independently selected R^{a}; —C(O)(C₁₋₄ alkyl); —C(O)O(C₁₋₄ alkyl); —CONR′R″; —S(O)₁₋₂NR′R″; —S(O)₁₋₂(C₁₋₄ alkyl); —OH; benzyl, and C₁₋₄ alkoxy;
each occurrence of R^{e} and R^{f} is independently selected from the group consisting of H; C₁₋₆ alkyl optionally substituted with 1-3 substituents each independently selected from the group consisting of NR′R″, —OH, halo, C₁₋₄ alkoxy, and C₁₋₄ haloalkoxy; —C(O)(C₁₋₄ alkyl); —C(O)O(C₁₋₄ alkyl); —CONR′R″; —S(O)₁₋₂NR′R″; —S(O)₁₋₂(C₁₋₄ alkyl); —OH; phenyl; and C₁₋₄ alkoxy;
each occurrence of R⁹ is independently selected from the group consisting of
   C₃₋₁₂ cycloalkyl or C₃₋₁₂ cycloalkenyl, each of which is optionally substituted with 1-4 substituents independently selected from the group consisting of oxo, R^{b}, and R^{h};
   heterocyclyl or heterocycloalkenyl of 3-12 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo, R^{b}, and R^{h};
   heteroaryl of 5-12 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of oxo, R^{e}, and R^{h}; and
   C₆₋₁₀ aryl optionally substituted with 1-4 substituents independently selected from the group consisting of oxo, R^{e}, and R^{h};
each occurrence of R^{h} is independently selected from the group consisting of
   C₃₋₁₂ cycloalkyl or C₃₋₁₂ cycloalkenyl, each of which is optionally substituted with 1-4 Rⁱ;
   heterocyclyl or heterocycloalkenyl of 3-12 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 Rⁱ;
   heteroaryl of 5-12 ring atoms, wherein 1-3 ring atoms are heteroatoms, each independently selected from the group consisting of N, N(H), N(R^{d}), O, and S(O)₀₋₂, and wherein the heteroaryl is optionally substituted with 1-4 Rⁱ; and
   C₆₋₁₀ aryl optionally substituted with 1-4 Rⁱ;
each occurrence of Rⁱ is independently selected from the group consisting of C₁₋₆ alkyl; C₁₋₄ haloalkyl; C₁₋₄ alkoxy; C₁₋₄ haloalkoxy; and halo; and
each occurrence of R′ and R″ is independently selected from the group consisting of H; —OH; and C₁₋₄ alkyl.

Also provided herein are compositions, e.g., pharmaceutical compositions, which include a compound of Formula (I), or a pharmaceutically acceptable salt thereof, or an atropisomer thereof; or a pharmaceutically acceptable salt of an atropisomer thereof and a pharmaceutically acceptable carrier.

This disclosure also features methods of using the compounds described herein, or a pharmaceutically acceptable salt thereof; or an atropisomer thereof; or a pharmaceutically acceptable salt of an atropisomer thereof, to modulate (e.g., inhibit) menin-MLL-interaction.

Methods include in vitro methods. By way of example, such methods include contacting a menin/MLL-containing sample (e.g., a biological sample) with a compound of formula (I), or a pharmaceutically acceptable salt thereof; or an atropisomer thereof; or a pharmaceutically acceptable salt of an atropisomer thereof.

Methods can also include in vivo methods. By way of example, such methods include administering a compound of formula (I), or a pharmaceutically acceptable salt thereof; or an atropisomer thereof; or a pharmaceutically acceptable salt of an atropisomer thereof, or a pharmaceutical composition thereof, to a subject (e.g., a human subject, e.g., a patient, e.g., a human patient). In some embodiments, the subject is suffering from a condition, disease or disorder in which aberrant (e.g., increased, e.g., excessive) menin-MLL interaction contributes to the pathology and/or symptoms and/or progression of the condition, disease or disorder (e.g., cancer, e.g., diabetes).

In some embodiments, the subject has cancer.

In some embodiments, the patient has diabetes.

Accordingly, in some embodiments, this disclosure provides methods for treating a disease, condition, or disorder that is causally related to the aberrant activity of a menin-MLL interaction in vivo.

For example, this disclosure features methods of treating cancer in a subject (e.g., a human subject, e.g., a patient, e.g., a human patient) in need of such treatment.

The methods include administering a compound of Formula (I), or a pharmaceutically acceptable salt thereof; or an atropisomer thereof, of a pharmaceutically acceptable salt of an atropisomer thereof, to the subject.

In some embodiments, the cancer is selected from the group consisting of acute lymphoblastic leukaemia, acute myeloid leukaemia, childhood medulloblastoma, chronic lymphocytic leukaemia, diffuse large B cell lymphoma, follicular lymphoma, glioblastoma, liver cancer, myelodysplastic syndrome, pancreatic cancer, prostate cancer, renal cell carcinoma, and triple negative breast cancer.

As another example, this disclosure features methods of treating diabetes (e.g., type 1 or type 2) in a subject (e.g., a human subject, e.g., a patient, e.g., a human patient) in need of such treatment. The methods include administering a compound of Formula (I), or a pharmaceutically acceptable salt thereof, or an atropisomer thereof; of a pharmaceutically acceptable salt of an atropisomer thereof, to the subject.

In certain embodiments, the methods further include administering one or more additional therapeutic agents.

In some embodiments, the subject is a human subject, e.g., a patient, e.g., a human patient.

In some embodiments, the subject has, or previously determined to have, an increase in activity of a menin-MLL interaction that is higher than a control level of said activity.

In some embodiments, the methods further include identifying, e.g., from a biological sample obtained from the subject, that the subject has said increase in activity.

Accordingly, the methods can further include determining whether a subject (e.g., a human subject) is eligible or a candidate for treatment with a compound of formula (I) or a pharmaceutically acceptable salt thereof, or an atropisomer thereof, of a pharmaceutically acceptable salt of an atropisomer thereof, or a pharmaceutical composition thereof.

In some embodiments, the methods can further include evaluating the response of a subject (e.g., a human subject) to a compound of formula (I) or a pharmaceutically acceptable salt thereof, or a pharmaceutical composition thereof.

### Definitions

To facilitate understanding of the disclosure set forth herein, a number of additional terms are defined below. Generally, the nomenclature used herein and the laboratory procedures in organic chemistry, medicinal chemistry, and pharmacology described herein are those well-known and commonly employed in the art. Unless defined otherwise, all technical and scientific terms used herein generally have the same meaning as commonly understood by one of ordinary skill in the art to which this disclosure belongs. Each of the patents, applications, published applications, and other publications that are mentioned throughout the specification and the attached appendices are incorporated herein by reference in their entireties.

### Sterochemistry

The compounds provided herein encompass various stereochemical forms.

The compounds described herein include all stereoisomers, including diastereoisomers, atropisomers, enantiomers, mixtures thereof and racemic mixtures, which arise because of structural asymmetry in certain compounds. Pharmaceutically acceptable salts thereof of these stereoisomeric forms are also included.

In some embodiments, compounds described herein, e.g., ortho-substituted biaryl compounds, may exhibit conformational, rotational isomerism, herein referred to as atropisomers (Eliel, E. and Wilen, S. (1994) Stereochemistry of Organic Compounds, John Wiley & Sons, Inc., pp. 1142-55). In some instances, depending upon the substituents on the bi-aryl ring moiety, such biaryl compounds described herein exhibit atropisomerism.

In some instances, the specification depicts and/or names structurally asymmetric compounds without specifying the stereochemistry (e.g., the stereochemistry at one or more chiral centers and/or one or more rotational isomers). In these cases, and for the avoidance of doubt, the chemical formulas and/or names represent all possible stereoisomers of the compound.

For certain compounds, the use of solid and dashed wedges or lines in conjunction with the symbols (R) and (S) at a chiral center denotes that this chiral center has been resolved (i.e., is a single epimer) and is intended to indicate absolute stereochemistry in accordance with the Cahn-Ingold-Prelog priority rules.

In some embodiments, the structurally asymmetric compounds described here are resolved using chiral HPLC. Unless otherwise indicated, the specification does not correlate absolute stereochemistry with peak retention time.

### Nitrogen Protecting Groups

As used herein, the term “nitrogen-protecting group” (PG) in a compound described herein to a group that protects a functional groups against unwanted secondary reactions, such as acylations, etherifications, esterifications, oxidations, solvolysis and similar reactions A variety of deprotection conditions are available for removing the protecting groups described here. Depending on the protecting group employ ed, the skilled person would know how to remove the protecting group to obtain the free amine NH₂ group by reference to known procedures. These include reference to organic chemistry textbooks and literature procedures such as J. F. W. McOmie, “Protective Groups in Organic Chemistry”, T. W. Greene and P. G. M. Wuts. “Greene's Protective Groups in Organic Synthesis”, and in “Methoden der organischen Chemie” (Methods of Organic Chemistry).

In some embodiments, nitrogen-protecting groups include C₁-C₆alkyl (e.g. tert-butyl), preferably C₁-C₄alkyl, more preferably C₁-C₂alkyl, most preferably C₁-alkyl which is mono-, di- or tri-substituted by trialkylsilyl-C₁-C₇alkoxy (e.g. trimethylsilylethoxy).

In some embodiments, nitrogen-protecting groups include aryl, preferably phenyl, or a heterocyclic group (e.g., benzyl, cumyl, benzhydryl, pyrrolidinyl, trityl, pyrrolidinylmethyl 1-methyl-1,1-dimethylbenzyl(phenyl)methylbenzene) wherein the aryl ring or the heterocyclic group is unsubstituted or substituted by one or more, e.g. two or three, residues, e.g. selected from the group consisting of C₁-C₇alkyl, hydroxy, C₁-C₇alkoxy (e.g. para-methoxy benzyl (PMB)), C₂-C₈-alkanoyl-oxy, halogen, nitro, cyano, and CF₃.

In some embodiments, nitrogen-protecting groups include aryl-C₁-C₂-alkoxycarbonyl (preferably phenyl-C₁-C₇-alkoxycarbonyl (e.g. benzyloxycarbonyl (Cbz), benzyloxymethyl (BOM), pivaloyloxymethyl (POM)), C₆-C₁₀-alkenyloxycarbonyl, C₁-C₆alkylcarbonyl (e.g. acetyl orpivaloyl), C₆-C₁₀-arylcarbonyl; C₁-C₆-alkoxycarbonyl (e.g. tert-butoxycarbonyl (Boc), methylcarbonyl trichloroethoxycarbonyl(Troc), pivaloyl (Piv), allyloxycarbonyl), C₆-C₁₀-arylC1-C₆-alkoxycarbonyl (e.g. 9-fluorenylmethyloxycarbonyl (Fmoc)), allyl or cinnamyl, sulfonyl or sulfenyl, succinimidyl group, silyl groups (e.g. triarylsilyl, trialkylsilyl, triethylsilyl (TES), trimethylsilylethoxymethyl (SEM), trimethylsilyl (TMS), triisopropylsilyl or tert-butyldimethylsilyl).

In embodiments, the nitrogen-protecting group is C₁-C₆-alkoxycarbonyl (e.g. tert-butoxycarbonyl (Boc), methyloxycarbonyl, trichloroethoxycarbonyl (Troc)-pivaloyl (Piv) allyloxycarbonyl), e.g., the nitrogen-protecting group can be tert-butoxycarbonyl.

In embodiments, the nitrogen-protecting group is a C₁-C₆-alkoxycarbonyl (e.g. tert-butoxycarbonyl or t-butyl carbamate (Boc), methyloxycarbonyl or methyl carbamate, ethyl carbamate, 9-fluorophenylmethyl carbamate (Fmoc) and analogs thereof, 2,2,2-trichloroethyl carbamate trichloroethoxycarbonyl (Troc), 2-trimethylsilylethyl carbamate (Teoc), pivaloyl (Piv), allyloxycarbonyl or allyl carbamate (Alloc), benzyl carbamate (Cbz)) or an amide protecting group e.g. COCF₃ (trifluoroacetamide), or N-allyl or N-benzyl and analogs thereof; e.g., the nitrogen-protecting group is tert-butoxycarbonyl.

In certain embodiments, the nitrogen protecting group is selected from Carbobenzyloxy (Cbz); p-Methoxybenzyl carbonyl (Moz or MeOZ); tert-Butyloxycarbonyl (BOC) group; 9-Fluorenylmethyloxycarbonyl (FMOC); Acetyl (Ac); Trifluoroacetyl; Benzoyl (Bz); Benzyl (Bn); Carbamates; p-Methoxybenzyl (PMB); 3,4-Dimethoxybenzyl (DMPM); p-methoxyphenyl (PMP) group; and Tosyl (Ts) group.

### Other Definitions

The term “acceptable” with respect to a formulation, composition or ingredient, as used herein, means having no persistent detrimental effect on the general health of the subject being treated.

The terms “effective amount” or “therapeutically effective amount,” as used herein, refer to a sufficient amount of a chemical entity being administered which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is determined using any suitable technique, such as a dose escalation study.

The term “excipient” or “pharmaceutically acceptable excipient” means a pharmaceutically-acceptable material, composition, or vehicle, such as a liquid or solid filler, diluent, carrier, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21st ed.; Lippincott Williams & Wilkins: Philadelphia, PA, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009.

The term “pharmaceutically acceptable salt” refers to a formulation of a compound that does not cause significant irritation to an organism to which it is administered and does not abrogate the biological activity and properties of the compound. In certain instances, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. In some instances, pharmaceutically acceptable salts are obtained by reacting a compound having acidic group described herein with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods previously determined. The pharmacologically acceptable salt s not specifically limited as far as it can be used in medicaments. Examples of a salt that the compounds described hereinform with a base include the following: salts thereof with inorganic bases such as sodium, potassium, magnesium, calcium, and aluminum; salts thereof with organic bases such as methylamine, ethylamine and ethanolamine; salts thereof with basic amino acids such as lysine and ornithine; and ammonium salt. The salts may be acid addition salts, which are specifically exemplified by acid addition salts with the following: mineral acids such as hydrochloric acid, hydrobromic acid, hydroiodic acid, sulfuric acid, nitric acid, and phosphoric acid:organic acids such as formic acid, acetic acid, propionic acid, oxalic acid, malonic acid, succinic acid, fumaric acid, maleic acid, lactic acid, malic acid, tartaric acid, citric acid, methanesulfonic acid, and ethanesulfonic acid; acidic amino acids such as aspartic acid and glutamic acid.

The term “pharmaceutical composition” refers to a mixture of a compound described herein with other chemical components (referred to collectively herein as “excipients”), such as carriers, stabilizers, diluents, dispersing agents, suspending agents, and/or thickening agents. The pharmaceutical composition facilitates administration of the compound to an organism. Multiple techniques of administering a compound exist in the art including, but not limited to: rectal, oral, intravenous, aerosol, parenteral, ophthalmic, pulmonary, and topical administration.

The term “subject” refers to an animal, including, but not limited to, a primate (e.g., human), monkey, cow, pig, sheep, goat, horse, dog, cat, rabbit, rat, or mouse. The terms “subject” and “patient” are used interchangeably herein in reference, for example, to a mammalian subject, such as a human.

The term “halo” refers to fluoro (F), chloro (Cl), bromo (Br), or iodo (I).

The term “oxo” refers to a divalent doubly bonded oxygen atom (i.e., “═O”). As used herein, oxo groups are attached to carbon atoms to form carbonyls.

The term “alkyl” refers to a saturated acyclic hydrocarbon radical that may be a straight chain or branched chain, containing the indicated number of carbon atoms. For example, C₁₋₁₀ indicates that the group may have from 1 to 10 (inclusive) carbon atoms in it. Alkyl groups can either be unsubstituted or substituted with one or more substituents. Non-limiting examples include methyl, ethyl, iso-propyl, tert-butyl, n-hexyl. The term “saturated” as used in this context means only single bonds present between constituent carbon atoms and other available valences occupied by hydrogen and/or other substituents as defined herein.

The term “haloalkyl” refers to an alkyl, in which one or more hydrogen atoms is/are replaced with an independently selected halo.

The term “alkoxy” refers to an —O-alkyl radical (e.g., —OCH₃).

The term “alkylene” refers to a divalent alkyl (e.g., —CH₂—). Similarly, terms such as “cycloalkylene” and “heterocyclylene” refer to divalent cycloalkyl and heterocyclyl respectively. For avoidance of doubt, in “cycloalkylene” and “heterocyclylene”, the two radicals can be on the same ring carbon atom (e.g., a geminal diradical such as

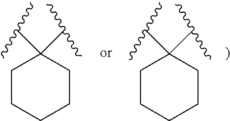 [SMILES: *C1(*)CCCCC1 ; *C1(*)CCCCC1]

or on different ring atoms (e.g., ring carbon and/or nitrogen atoms (e.g., vicinal ring carbon and/or nitrogen atoms)) (e.g.,

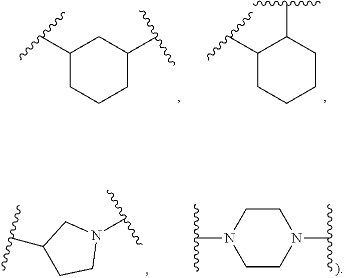 [SMILES: *C1CCCC(*)C1 ; *C1CCCCC1* ; *C1CCN(*)C1 ; *N1CCN(*)CC1]

The term “alkenyl” refers to an acyclic hydrocarbon chain that may be a straight chain or branched chain having one or more carbon-carbon double bonds. The alkenyl moiety contains the indicated number of carbon atoms. For example, C₂₋₆ indicates that the group may have from 2 to 6 (inclusive) carbon atoms in it. Alkenyl groups can either be unsubstituted or substituted with one or more substituents.

The term “alkenylene” refers to divalent alkenyl.

The term “alkynyl” refers to an acyclic hydrocarbon chain that may be a straight chain or branched chain having one or more carbon-carbon triple bonds. The alkynyl moiety contains the indicated number of carbon atoms. For example, C₂₋₆ indicates that the group may have from 2 to 6 (inclusive) carbon atoms in it. Alkynyl groups can either be unsubstituted or substituted with one or more substituents.

The term “alkynylene” refers to divalent alkynyl.

The term “aryl” refers to a 6-20 carbon mono-, bi-, tri- or polycyclic group wherein at least one ring in the system is aromatic (e.g., 6-carbon monocyclic, 10-carbon bicyclic, or 14-carbon tricyclic aromatic ring system); and wherein 0, 1, 2, 3, or 4 atoms of each ring may be substituted by a substituent. Examples of aryl groups include phenyl, naphthyl, tetrahydronaphthyl, and the like.

The term “cycloalkyl” as used herein refers to cyclic saturated hydrocarbon groups having, e.g., 3 to 20 ring carbons, preferably 3 to 16 ring carbons, and more preferably 3 to 12 ring carbons or 3-10 ring carbons or 3-6 ring carbons, wherein the cycloalkyl group may be optionally substituted. Examples of cycloalkyl groups include, without limitation, cyclopropyl, cyclobutyl, cyclopentyl, cyclohexyl, cycloheptyl, and cyclooctyl. Cycloalkyl may include multiple fused and/or bridged rings. Non-limiting examples of fused/bridged cycloalkyl includes: bicyclo[1.1.0]butane, bicyclo[2.1.0]pentane, bicyclo[1.1.1]pentane, bicyclo[3.1.0]hexane, bicyclo[2.1.1]hexane, bicyclo[3.2.0]heptane, bicyclo[4.1.0]heptane, bicyclo[2.2.1]heptane, bicyclo[3.1.1]heptane, bicyclo[4.2.0]octane, bicyclo[3.2.1]octane, bicyclo[2.2.2]octane, and the like. Cycloalkyl also includes spirocyclic rings (e.g., spirocyclic bicycle wherein two rings are connected through just one atom). Non-limiting examples of spirocyclic cycloalkyls include spiro[2.2]pentane, spiro[2.5]octane, spiro[3.5]nonane, spiro[3.5]nonane, spiro[3.5]nonane, spiro[4.4]nonane, spiro[2.6]nonane, spiro[4.5]decane, spiro[3.6]decane, spiro[5.5]undecane, and the like. The term “saturated” as used in this context means only single bonds present between constituent carbon atoms.

The term “cycloalkenyl” as used herein means partially unsaturated cyclic hydrocarbon groups having 3 to 20 ring carbons, preferably 3 to 16 ring carbons, and more preferably 3 to 12 ring carbons or 3-10 ring carbons or 3-6 ring carbons, wherein the cycloalkenyl group may be optionally substituted. Examples of cycloalkenyl groups include, without limitation, cyclopentenyl, cyclohexenyl, cycloheptenyl, and cyclooctenyl. As partially unsaturated cyclic hydrocarbon groups, cycloalkenyl groups may have any degree of unsaturation provided that one or more double bonds is present in the ring, none of the rings in the ring system are aromatic, and the cycloalkenyl group is not fully saturated overall. Cycloalkenyl may include multiple fused and/or bridged and/or spirocyclic rings.

The term “heteroaryl”, as used herein, means a mono-, bi-, tri- or polycyclic group having 5 to 20 ring atoms, alternatively 5, 6, 9, 10, or 14 ring atoms; wherein at least one ring in the system contains one or more heteroatoms independently selected from the group consisting of N, O, and S and at least one ring in the system is aromatic (but does not have to be a ring which contains a heteroatom, e.g. tetrahydroisoquinolinyl, e.g., tetrahydroquinolinyl). Heteroaryl groups can either be unsubstituted or substituted with one or more substituents. Examples of heteroaryl include thienyl, pyridinyl, furyl, oxazolyl, oxadiazolyl, pyrrolyl, imidazolyl, triazolyl, thiodiazolyl, pyrazolyl, isoxazolyl, thiadiazolyl, pyranyl, pyrazinyl, pyrimidinyl, pyridazinyl, triazinyl, thiazolyl benzothienyl, benzoxadiazolyl, benzofuranyl, benzimidazolyl, benzotriazolyl, cinnolinyl, indazolyl, indolyl, isoquinolinyl, isothiazolyl, naphthyridinyl, purinyl, thienopyridinyl, pyrido[2,3-d]pyrimidinyl, pyrrolo[2,3-b]pyridinyl, quinazolinyl, quinolinyl, thieno[2,3-c]pyridinyl, pyrazolo[3,4-b]pyridinyl, pyrazolo[3,4-c]pyridinyl, pyrazolo[4,3-c]pyridine, pyrazolo[4,3-b]pyridinyl, tetrazolyl, chromane, 2,3-dihydrobenzo[b][1,4]dioxine, benzo[d][1,3]dioxole, 2,3-dihydrobenzofuran, tetrahydroquinoline, 2,3-dihydrobenzo[b][1,4]oxathiine, isoindoline, and others. In some embodiments, the heteroaryl is selected from thienyl, pyridinyl, furyl, pyrazolyl, imidazolyl, isoindolinyl, pyranyl, pyrazinyl, and pyrimidinyl. For purposes of clarification, heteroaryl also includes aromatic lactams, aromatic cyclic ureas, or vinylogous analogs thereof, in which each ring nitrogen adjacent to a carbonyl is tertiary (i.e., all three valences are occupied by non-hydrogen substituents), such as one or more of pyridone

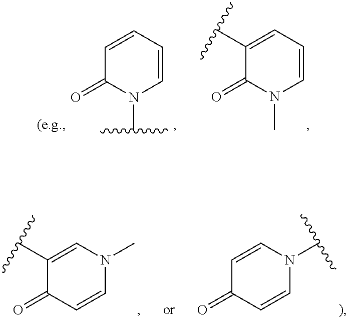

pyrimidone

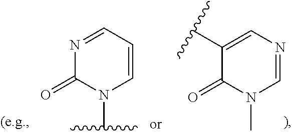

pyridazinone

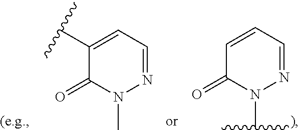

pyrazinone

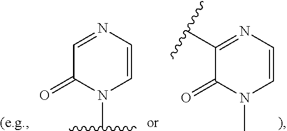

and imidazolone

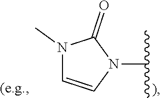 [SMILES: *n1ccn(C)c1=O]

wherein each ring nitrogen adjacent to a carbonyl is tertiary (i.e., the oxo group (i.e., “═O”) herein is a constituent part of the heteroaryl ring).

The term “heterocyclyl” refers to a mono-, bi-, tri-, or polycyclic saturated ring system with 3-16 ring atoms (e.g., 5-8 membered monocyclic, 8-12 membered bicyclic, or 11-14 membered tricyclic ring system) having 1-3 heteroatoms if monocyclic, 1-6 heteroatoms if bicyclic, or 1-9 heteroatoms if tricyclic or polycyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2 or 3 atoms of each ring may be substituted by a substituent. Examples of heterocyclyl groups include piperazinyl, pyrrolidinyl, dioxanyl, morpholinyl, tetrahydrofuranyl, and the like. Heterocyclyl may include multiple fused and bridged rings. Non-limiting examples of fused/bridged heterocyclyl includes: 2-azabicyclo[1.1.0]butane, 2-azabicyclo[2.1.0]pentane, 2-azabicyclo[1.1.1]pentane, 3-azabicyclo[3.1.0]hexane, 5-azabicyclo[2.1.1]hexane, 3-azabicyclo[3.2.0]heptane, octahydrocyclopenta[c]pyrrole, 3-azabicyclo[4.1.0]heptane, 7-azabicyclo[2.2.1]heptane, 6-azabicyclo[3.1.1]heptane, 7-azabicyclo[4.2.0]octane, 2-azabicyclo[2.2.2]octane, 3-azabicyclo[3.2.1]octane, 2-oxabicyclo[1.1.0]butane, 2-oxabicyclo[2.1.0]pentane, 2-oxabicyclo[1.1.1]pentane, 3-oxabicyclo[3.1.0]hexane, 5-oxabicyclo[2.1.1]hexane, 3-oxabicyclo[3.2.0]heptane, 3-oxabicyclo[4.1.0]heptane, 7-oxabicyclo[2.2.1]heptane, 6-oxabicyclo[3.1.1]heptane, 7-oxabicyclo[4.2.0]octane, 2-oxabicyclo[2.2.2]octane, 3-oxabicyclo[3.2.1]octane, and the like. Heterocyclyl also includes spirocyclic rings (e.g., spirocyclic bicycle wherein two rings are connected through just one atom). Non-limiting examples of spirocyclic heterocyclyls include 2-azaspiro[2.2]pentane, 4-azaspiro[2.5]octane, 1-azaspiro[3.5]nonane, 2-azaspiro[3.5]nonane, 7-azaspiro[3.5]nonane, 2-azaspiro[4.4]nonane, 6-azaspiro[2.6]nonane, 1,7-diazaspiro[4.5]decane, 7-azaspiro[4.5]decane 2,5-diazaspiro[3.6]decane, 3-azaspiro[5.5]undecane, 2-oxaspiro[2.2]pentane, 4-oxaspiro[2.5]octane, 1-oxaspiro[3.5]nonane, 2-oxaspiro[3.5]nonane, 7-oxaspiro[3.5]nonane, 2-oxaspiro[4.4]nonane, 6-oxaspiro[2.6]nonane, 1,7-dioxaspiro[4.5]decane, 2,5-dioxaspiro[3.6]decane, 1-oxaspiro[5.5]undecane, 3-oxaspiro[5.5]undecane, 3-oxa-9-azaspiro[5.5]undecane and the like. The term “saturated” as used in this context means only single bonds present between constituent ring atoms and other available valences occupied by hydrogen and/or other substituents as defined herein.

The term “heterocycloalkenyl” as used herein means partially unsaturated cyclic ring system with 3-16 ring atoms (e.g., 5-8 membered monocyclic, 8-12 membered bicyclic, or 11-14 membered tricyclic ring system) having 1-3 heteroatoms if monocyclic, 1-6 heteroatoms if bicyclic, or 1-9 heteroatoms if tricyclic or polycyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2 or 3 atoms of each ring may be substituted by a substituent. Examples of heterocycloalkenyl groups include, without limitation, tetrahydropyridyl, dihydropyrazinyl, dihydropyridyl, dihydropyrrolyl, dihydrofuranyl, dihydrothiophenyl.

As partially unsaturated cyclic groups, heterocycloalkenyl groups may have any degree of unsaturation provided that one or more double bonds is present in the ring, none of the rings in the ring system are aromatic, and the heterocycloalkenyl group is not fully saturated overall. Heterocycloalkenyl may include multiple fused and/or bridged and/or spirocyclic rings.

As used herein, examples of aromatic rings include: benzene, pyridine, pyrimidine, pyrazine, pyridazine, pyridone, pyrrole, pyrazole, oxazole, thioazole, isoxazole, isothiazole, and the like.

As used herein, when a ring is described as being “partially unsaturated”, it means said ring has one or more additional degrees of unsaturation (in addition to the degree of unsaturation attributed to the ring itself; e.g., one or more double or triple bonds between constituent ring atoms), provided that the ring is not aromatic. Examples of such rings include: cyclopentene, cyclohexene, cycloheptene, dihydropyridine, tetrahydropyridine, dihydropyrrole, dihydrofuran, dihydrothiophene, and the like.

For the avoidance of doubt, and unless otherwise specified, for rings and cyclic groups (e.g., aryl, heteroaryl, heterocyclyl, heterocycloalkenyl, cycloalkenyl, cycloalkyl, and the like described herein) containing a sufficient number of ring atoms to form bicyclic or higher order ring systems (e.g., tricyclic, polycyclic ring systems), it is understood that such rings and cyclic groups encompass those having fused rings, including those in which the points of fusion are located (i) on adjacent ring atoms (e.g., [x.x.0] ring systems, in which 0 represents a zero atom bridge

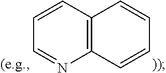

(ii) a single ring atom (spiro-fused ring systems)

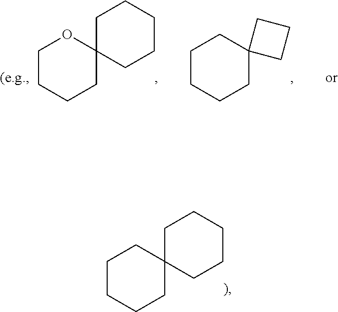

or (iii) a contiguous array of ring atoms (bridged ring systems having all bridge lengths>0)

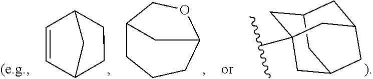

In addition, atoms making up the compounds of the present embodiments are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

In addition, the compounds generically or specifically disclosed herein are intended to include all tautomeric forms. Thus, by way of example, a compound containing the moiety:

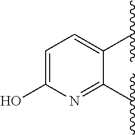 [SMILES: *c1ccc(O)nc1*]

encompasses the tautomeric form containing the moiety:

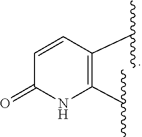 [SMILES: *c1ccc(=O)[nH]c1*]

Similarly, a pyridinyl or pyrimidinyl moiety that is described to be optionally substituted with hydroxyl encompasses pyridone or pyrimidone tautomeric forms.

The details of one or more embodiments of the invention are set forth in the accompanying drawings and the description below. Other features and advantages will be apparent from the description and drawings, and from the claims

### DESCRIPTION OF DRAWINGS

FIGS. 1A and 1B show efficacy study in menin(WT) MV-4-11 subcutaneous model (WT SC) in linear and logarithmic scales, respectively. Statistical analysis conducted comparing the vehicle to the different treatment groups; p≤0.05=*, p≤0.02=**, p≤0.0002=***, p≤0.0001=****.

FIGS. 2A and 2B show efficacy study in menin(M327I) homozygous mutant MV-4-11 subcutaneous model (M327I SC) in linear and logarithmic scales respectively. Statistical analysis conducted comparing the vehicle to the different treatment groups; p≤0.05=*, p≤0.02=**, p≤0.0002=***, p≤0.0001=****.

### DETAILED DESCRIPTION

This disclosure provides chemical entities (e.g., a compound, or a pharmaceutically acceptable salt thereof, or an atropisomer thereof; or a pharmaceutically acceptable salt of an atropisomer) that modulate (e.g., inhibit) the interaction between menin and mixed-lineage leukaemia (“MLL”) proteins (e.g., MLL fusion proteins). The chemical entities are useful, e.g., for treating a subject (e.g., a human) having a condition, disease or disorder in which aberrant (e.g., increased, e.g., excessive) menin-MLL interaction contribute to the pathology, symptoms and/or progression of the condition, disease or disorder (e.g., cancer, diabetes). This disclosure also provides compositions containing the chemical entities as well as methods of making and using the same. Advantageously, and without wishing to be bound by theory, it is believed that the chemical entities described herein can achieve equipotency and efficacy in the presence of both WT and mutant menin.

### Compound Embodiments of Formula (I)

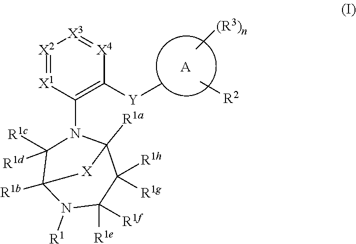

Variables R^{1a}, R^{1b}, R^{1c}, R^{1d}, R^{1e}, R^{1f}, R^{1g}, R^{1h} and X

In some embodiments, each of R^{1a} and R^{1b} is H.

In some embodiments, X is CH₂.

In some embodiments, each of R^{1c}, R^{1d}, R^{1e}, R^{1f}, R^{1g}, and R^{1h} is H.

In some embodiments, the compound has Formula (I-A):

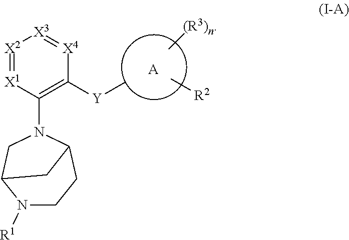

In some embodiments, the compound has Formula (I-B):

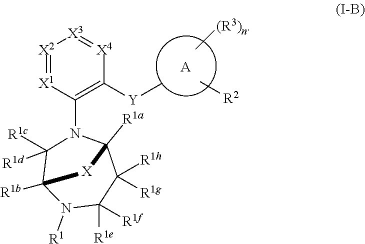

In some embodiments of Formula (I-B), each of R^{1a} and R^{1b} is H.

In some embodiments of Formula (I-B), X is CH₂.

In some embodiments of Formula (I-B), each of R^{1c}, R^{1d}, R^{1e}, R^{1f}, R^{1g}, and R^{1b} is H.

In some embodiments, the compound has Formula (I-C):

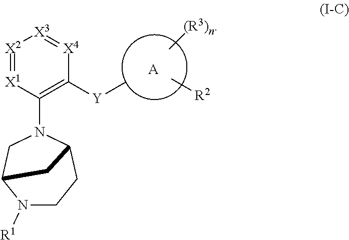

### Variable R¹

In some embodiments of Formula (I), (I-A), (I-B), and/or (I-C), R¹ is

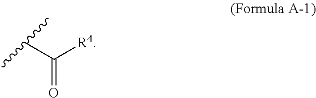

In some embodiments of Formula (A-1), R⁴ is:
C₁₋₈ alkyl or C₂₋₈ alkenyl, each of which is optionally substituted with 1-3 independently selected R^{a},
L₁-C₃₋₁₀ cycloalkyl or C₃₋₁₀ cycloalkenyl, each of which is optionally substituted with 1-4 substituents independently selected from the group consisting of oxo, R^{b}, and R^{g};
L₁-heterocyclyl or L₁-heterocycloalkenyl of 3-12 ring atoms, wherein 1-3 ring atoms are heteroatoms, each independently selected from the group consisting of N, N(H), N(R^{d}), N(O⁻), O, and S(O)₀₋₂, and wherein the heterocyclyl or heterocycloalkenyl is optionally substituted with 1-4 substituents independently selected from the group consisting of oxo, R^{b}, and R^{g}; or
L₁-heteroaryl of 5-12 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of oxo, R^{c}, and R⁹.

In certain embodiments of Formula (A-1), R⁴ is:
C₁₋₈ alkyl, which is optionally substituted with 1-3 independently selected R^{a}, or
L₁-C₃₋₁₀ cycloalkyl, which is optionally substituted with 1-4 substituents independently selected from the group consisting of oxo, R^{b}, and R^{g};
L₁-heterocyclyl of 3-12 ring atoms, wherein 1-3 ring atoms are heteroatoms, each independently selected from the group consisting of N, N(H), N(R^{d}), N(O⁻), O, and S(O)₀₋₂, and wherein the heterocyclyl or heterocycloalkenyl is optionally substituted with 1-4 substituents independently selected from the group consisting of oxo, R^{b}, and R^{g}; or
L₁-heteroaryl of 5-12 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of oxo, R^{c}, and R⁹.

In certain embodiments, R⁴ in Formula A-1 is L₁-heterocyclyl or L₁-heterocycloalkenyl of 3-12 ring atoms, wherein 1-3 ring atoms are heteroatoms, each independently selected from the group consisting of N, N(H), N(R^{d}), N(O⁻), O, and S(O)₀₋₂, and wherein the heterocyclyl or heterocycloalkenyl is optionally substituted with 1-4 substituents independently selected from the group consisting of oxo, R^{b}, and R^{g}.

In certain embodiments, R⁴ in Formula A-1 is L₁-heterocyclyl of 3-12 ring atoms, wherein 1-3 ring atoms are heteroatoms, each independently selected from the group consisting of N(H), N(R^{d}), N(O⁻), O, and S(O)₀₋₂, and wherein the heterocyclyl is optionally substituted with 1-4 substituents independently selected from the group consisting of oxo, R^{b}, and R^{g}.

In certain embodiments, R⁴ in Formula A-1 is L₁-heterocyclyl of 3-8 ring atoms, wherein 1-2 ring atoms are heteroatoms, each independently selected from the group consisting of N(H), N(R^{d}), N(O—), O, and S(O)₀₋₂, and wherein the heterocyclyl is optionally substituted with 1-4 substituents independently selected from the group consisting of oxo, R^{b}, and R^{g}.

In certain embodiments, R⁴ in Formula A-1 is L₁-heterocyclyl of 3-8 ring atoms, wherein 1-2 ring atoms are heteroatoms, each independently selected from the group consisting of N(H), N(R^{d}), N(O⁻), and O, and wherein the heterocyclyl is optionally substituted with 1-4 substituents independently selected from the group consisting of oxo, R^{b}, and R^{g}

In certain of the foregoing Formula A-1 embodiments, the heterocyclyl in Formula A-1 is monocyclic.

In certain embodiments, R⁴ in Formula A-1 is L₁-monocyclic heterocyclyl of 4-6 ring atoms, wherein 1-2 ring atoms are heteroatoms, each independently selected from the group consisting of N(H), N(R^{d}), N(O⁻), O, and S(O)₀₋₂, which is optionally substituted with 1-4 substituents independently selected from the group consisting of oxo, R^{b}, and R^{g}.

In certain embodiments, R⁴ in Formula A-1 is L₁-monocyclic heterocyclyl of 4-6 ring atoms, wherein 1-2 ring atoms are heteroatoms, each independently selected from the group consisting of N(H), N(R^{d}), N(O—), and O, which is optionally substituted with 1-4 substituents independently selected from the group consisting of oxo, R^{b}, and R^{g}.

For example, R⁴ in Formula A-1 can be selected from the group consisting of L₁-oxetanyl, L₁-azetidinyl, L₁-thietanyl, L₁-thietanyl-1,1-dioxide, L₁-piperidinyl, L₁-piperazinyl, L₁-pyrrolidinyl, L₁-dioxanyl, L₁-morpholinyl, L₁-tetrahydrofuranyl, L₁-tetrehydrothienyl, and L₁-tetrehydrothienyl-1,1-dioxide, each of which is optionally substituted with 1-4 substituents independently selected from the group consisting of oxo, R^{b}, and R^{g}; optionally wherein R⁴ is L₁-pyrrolidinyl, which is optionally substituted with 1-4 substituents independently selected from the group consisting of oxo, R^{b}, and R^{g}; (and/or an N-oxide thereor); optionally wherein R⁴ is 2-methylpyrolidin-2-yl (or an N-oxide thereof).

In certain of the foregoing Formula A-1 embodiments, the heterocyclyl in Formula A-1 is bicyclic.

In certain embodiments, R⁴ in Formula A-1 is L₁-bicyclic heterocyclyl of 4-12 ring atoms, wherein 1-3 ring atoms are heteroatoms, each independently selected from the group consisting of N, N(H), N(R^{d}), N(O⁻), O, and S(O)₀₋₂, and wherein the heterocyclyl is optionally substituted with 1-4 substituents independently selected from the group consisting of oxo, R^{b}, and R^{g} (and/or an N-oxide thereor).

In certain embodiments, R⁴ in Formula A-1 is L₁-bicyclic heterocyclyl of 6-10 ring atoms, wherein 1-3 ring atoms are heteroatoms, each independently selected from the group consisting of N, N(H), N(R^{d}), N(O⁻), O, and S(O)₀₋₂, and wherein the heterocyclyl is optionally substituted with 1-4 substituents independently selected from the group consisting of oxo, R^{b}, and R^{g} (and/or an N-oxide thereor).

In certain embodiments, R⁴ in Formula A-1 is L₁-bicyclic heterocyclyl of 6-8 ring atoms, wherein 1-3 ring atoms are heteroatoms, each independently selected from the group consisting of N, N(H), N(R^{d}), N(O⁻), O, and S(O)₀₋₂, and wherein the heterocyclyl is optionally substituted with 1-4 substituents independently selected from the group consisting of oxo, R^{b}, and R^{g} (and/or an N-oxide thereor).

In certain embodiments, R⁴ in Formula A-1 can be selected from the group consisting of L₁-1-azabicyclo[1.1.1]pentanyl, L₁-2-azabicyclo[3.1.0]hexanyl, L₁-3-azabicyclo[3.1.0]hexanyl, L₁-2-azabicyclo[2.1.1]hexanyl, L₁-5-azabicyclo[2.1.1]hexanyl, L₁-3-azabicyclo[3.2.0]heptanyl, L₁-octahydrocyclopenta[c]pyrrolyl, L₁-3-azabicyclo[4.1.0]heptanyl, L₁-7-azabicyclo[2.2.1]heptanyl, L₁-6-azabicyclo[3.1.1]heptanyl, L₁-7-azabicyclo[4.2.0]octanyl, L₁-2-azabicyclo[2.2.2]octanyl, L₁-3-azabicyclo[3.2.1]octanyl, L₁-3-azabicyclo[3.3.0]octanyl, L₁-hexahydro-1H-pyrrolizinyl, L₁-5-azaspiro[2.4]heptanyl, L₁-4-azaspiro[2.4]heptanyl, L₁-azaspiro[2.5]octanyl, L₁-1-azaspiro[3.5]nonanyl, L₁-2-azaspiro[3.5]nonanyl, L₁-7-azaspiro[3.5]nonanyl, L₁-2-azaspiro[4.4]nonanyl, L₁-6-azaspiro[2.6]nonanyl, L₁-1,7-diazaspiro[4.5]decanyl, L₁-7-azaspiro[4.5]decanyl L₁-2,5-diazaspiro[3.6]decanyl, and L₁-3-azaspiro[5.5]undecanyl, each of which is optionally substituted with 1-4 substituents independently selected from the group consisting of oxo, R^{b}, and R^{g} (and/or an N-oxide thereof)

In certain embodiments, R⁴ in Formula A-1 is selected from the group consisting of L₁-2-azabicyclo[3.1.0]hexanyl, L₁-3-azabicyclo[3.1.0]hexanyl, L₁-2-azabicyclo[2.1.1]hexanyl, L₁-5-azabicyclo[2.1.1]hexanyl, L₁-5-azaspiro[2.4]heptanyl, L₁-3-azabicyclo[3.3.0]octanyl, L₁-3-azabicyclo[3.3.0]octanyl, L₁-hexahydro-1H-pyrrolizinyl, and L₁-4-azaspiro[2.4]heptanyl, each of which is optionally substituted with 1-4 substituents independently selected from the group consisting of oxo, R^{b}, and R^{g} (and/or an N-oxide thereof).

For example, R⁴ in Formula A-1 is selected from the group consisting of

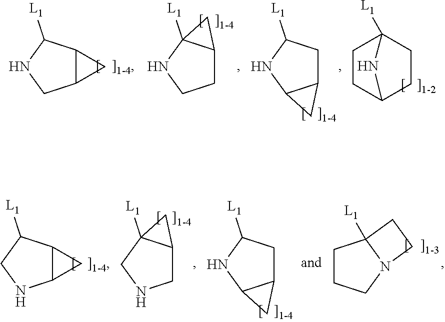

which is optionally substituted with 1-4 substituents independently selected from the group consisting of oxo, R^{b}, and R^{g} (and/or an N-oxide thereof).

As another example, R⁴ in Formula A-1 is selected from the group consisting of:

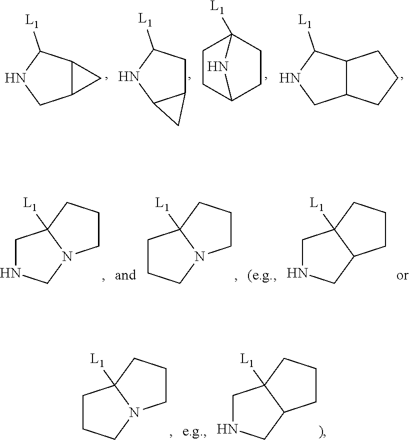

each of which is optionally substituted with 1-4 substituents independently selected from the group consisting of oxo, R^{b}, and R^{g} (and/or an N-oxide thereof).

As a further example, R⁴ in Formula A-1 has one of the following formulas:

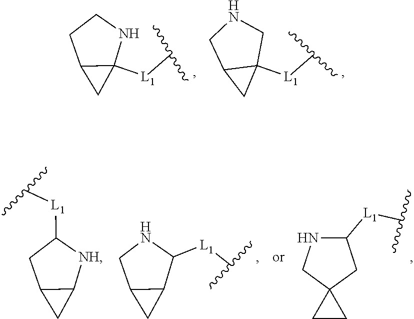

each of which is optionally substituted with 1-4 substituents independently selected from the group consisting of oxo, R^{b}, and R^{g} (and/or an N-oxide thereof).

In some of the Formula A-1 embodiments, each occurrence of R^{b} is, independently, selected from the group consisting of: halo; C₁-10 alkyl which is optionally substituted with 1-6 independently selected R^{a}; C₁₋₄ alkoxy; C₁₋₄ haloalkoxy; —NR^{e}R^{f}; —OH; and —C₁₋₄ thioalkoxy.

In some of the Formula A-1 embodiments, each occurrence of R^{b} is, independently, selected from the group consisting of CH₃, OCH₃, F, CH₂F, and OH.

In some embodiments, R⁴ in Formula A-1 is C₁₋₈ alkyl, which is optionally substituted with 1-3 independently selected R^{a} In certain embodiments, R⁴ in Formula A-1 is C₁₋₄ alkyl, which is optionally substituted with 1-2 independently selected R^{a} In certain embodiments, wherein R⁴ in Formula A-1 is C₁₋₄ alkyl, which is optionally substituted with 1 R^{a} In certain of these Formula A-1 embodiments, each occurrence of R^{a} is NR^{e}R^{f}; optionally wherein one of R^{e} and R^{f} is H, and the other is C₁-C₃ alkyl.

For example, each occurrence of R^{a} can be NH₂ or NH(CH₃).

In certain of these Formula A-1 embodiments, R⁴ is unsubstituted C₁₋₄ alkyl.

In certain of these Formula A-1 embodiments, the substituted or unsubstituted C₁₋₄ alkyl is a straight chain C₁₋₄ alkyl.

In other of these Formula A-1 embodiments, the substituted or unsubstituted C₁-4 alkyl is a branched chain C₁₋₄ alkyl.

For example, the branched chain C₁₋₄ alkyl can include —*CH(CH₃)— as part of its structure. In certain instances, the*C has the (R)-configuration. In other instances, the *C has the (S)-configuration.

In some embodiments, R⁴ in Formula A-1 is L₁-C₃₋₁₀ cycloalkyl, which is optionally substituted with 1-4 substituents independently selected from the group consisting of oxo, R^{b}, and R^{g}.

In certain embodiments, R⁴ in Formula A-1 is L₁-C₃₋₆ cycloalkyl, which is optionally substituted with 1-4 substituents independently selected from the group consisting of oxo, R^{b}, and R^{g}.

For example, R⁴ in Formula A-1 is L₁-cyclopropyl or L₁-cyclobutyl, each of which is optionally substituted with 1-4 substituents independently selected from the group consisting of oxo, R^{b}, and R^{g}.

In some embodiments, R⁴ in Formula A-1 is L₁-heteroaryl of 5-12 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of oxo, R^{e}, and R⁹.

In certain embodiments, R⁴ in Formula A-1 is L₁-heteroaryl of 5-6 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of oxo, R^{e}, and R⁹.

For example, R⁴ in Formula A-1 is L₁-thienyl, L₁-pyridinyl, L₁-furyl, L₁-oxazolyl, L₁-oxadiazolyl, L₁-pyrrolyl, L₁-imidazolyl, L₁-triazolyl, L₁-thiodiazolyl, L₁-pyrazolyl, L₁-isoxazolyl, L₁-thiadiazolyl, L₁-pyrazinyl, L₁-pyrimidinyl, L₁-pyridazinyl, and L₁-triazinyl.

In some of the foregoing Formula A-1 embodiments, L₁ is a bond.

In certain of these embodiments, a ring carbon atom serves as the point of connection of the cycloalkyl, cycloalkenyl, heterocyclyl, heterocycloalkenyl, heteroaryl, or aryl ring to the C═O in Formula A-1.

In some embodiments, R¹ has the formula:

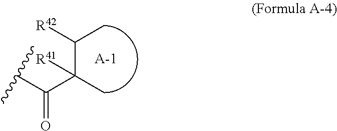

wherein:
R⁴¹ is H or R^{b}; and
R⁴² is H or R^{d}; and

Ring A-1 is an optionally substituted heterocyclyl or an optionally substituted heterocycloalkenyl having 4-8 total ring atoms; optionally having 4-6 total ring atoms, or optionally having five total ring atoms.

In some of the Formula A-4 embodiments, R⁴¹ is R^{b}.

In some of the Formula A-4 embodiments, R⁴¹ is C₁-C₂ alkyl.

For example, R⁴¹ can be CH₃.

As another example, R⁴¹ can be H.

In certain of the Formula A-4 embodiments, R¹ is:

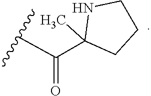 [SMILES: *C(=O)C1(C)CCCN1]

In embodiments, the carbon atom attached to the CH₃ in the formula above has the (R)-configuration.

In embodiments, the carbon atom attached to the CH₃ in the formula above has (S)-configuration.

In some of the foregoing Formula A-1 embodiments, L₁ is C₁₋₄ alkylene.

In certain of these Formula A-1 embodiments, a ring carbon atom serves as the point of connection of the cycloalkyl, cycloalkenyl, heterocyclyl, heterocycloalkenyl, heteroaryl, or aryl ring to L₁.

In certain of these Formula A-1 embodiments, the C₁₋₄ alkylene is a straight chain alkylene.

In other of these Formula A-1 embodiments, C₁₋₄ alkylene is a branched chain alkylene.

For example, the branched chain alkylene can incude *CH(CH₃)— as part of its structure. In some instances, the *C has the (R)-configuration. In other instances, the *C has the (S)-configuration.

In some embodiments of Formula (I), (I-A), (I-B), and/or (I-C), R¹ is H.

In some embodiments of Formula (I), (I-A), (I-B), and/or (I-C), R¹ is a nitrogen protecting group.

### Variable R²

In some embodiments of Formula (I), (I-A), (I-B), and/or (I-C), R² is:

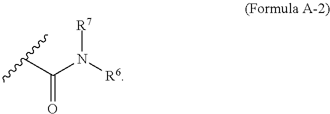

In some of the formula A-2 embodiments, each of R¹ and R⁷ is an independently selected C₁₋₄ alkyl. For example, one of R⁶ and R⁷ is —CH₂CH₃, and the other is —CH(CH₃)₂.

In some embodiments of Formula (I), (I-A), (I-B), and/or (I-C), R² is:

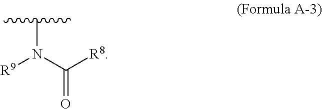

In some of the formula A-3 embodiments, each of R⁸ and R⁹ is an independently selected C₁₋₄ alkyl. For example, one of R⁶ and R⁷ is —CH₂CH₃, and the other is —CH(CH₃)₂.

In some embodiments of Formula (I), (I-A), (I-B), and/or (I-C), R² is R¹⁰.

In some of these embodiments. R¹⁰ is L₁-heteroaryl of 5-12 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of oxo, R^{e}, and R⁹.

In certain of these embodiments, R¹⁰ is L₁-heteroaryl of 5-6 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of oxo, R^{e}, and R⁹.

In certain of these embodiments, R¹⁰ can be selected from the group consisting of L₁-thienyl, L₁-pyridinyl, L₁-furyl, L₁-oxazolyl, L₁-oxadiazolyl, L₁-pyrrolyl, L₁-imidazolyl, L₁-triazolyl, L₁-thiodiazolyl, L₁-pyrazolyl, L₁-isoxazolyl, L₁-thiadiazolyl, L₁-pyrazinyl, L₁-pyrimidinyl, L₁-pyridazinyl, and L₁-triazinyl, each of which is optionally substituted with 1-4 substituents independently selected from the group consisting of oxo, R^{e}, and R⁹. In certain of these embodiments, L₁ is a bond.

For example, R¹⁰ can be selected from the group consisting of:

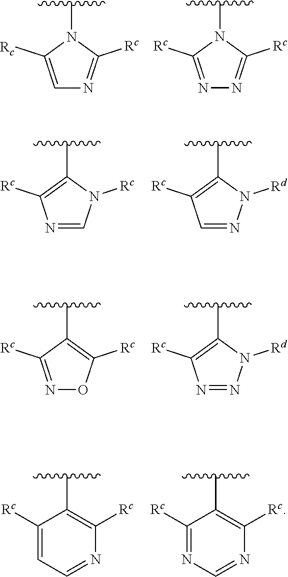

In some of the foregoing R¹⁰ embodiments, each R^{c} is, independently, C₁₋₄ alkyl, C₁₋₄ haloalkyl, or C₃₋₆ cycloalkyl.

In some of the foregoing R¹⁰ embodiments, each R^{c} is, independently, C₁₋₄ alkyl, or C₃₋₆ cycloalkyl.

In some of the foregoing R¹⁰ embodiments, each R^{c} is, independently, —CH₃, —CH(CH₃)₂, or cyclopropyl.

In some of the foregoing R¹⁰ embodiments, each R^{d} is, independently, C₁₋₄ alkyl or C₃₋₆ cycloalkyl.

In some of the foregoing R¹⁰ embodiments, each R^{c} is, independently, —CH₃ or cyclopropyl.

For example, in some of the foregoing R¹⁰ embodiments, R¹⁰ is selected from the group consisting of:

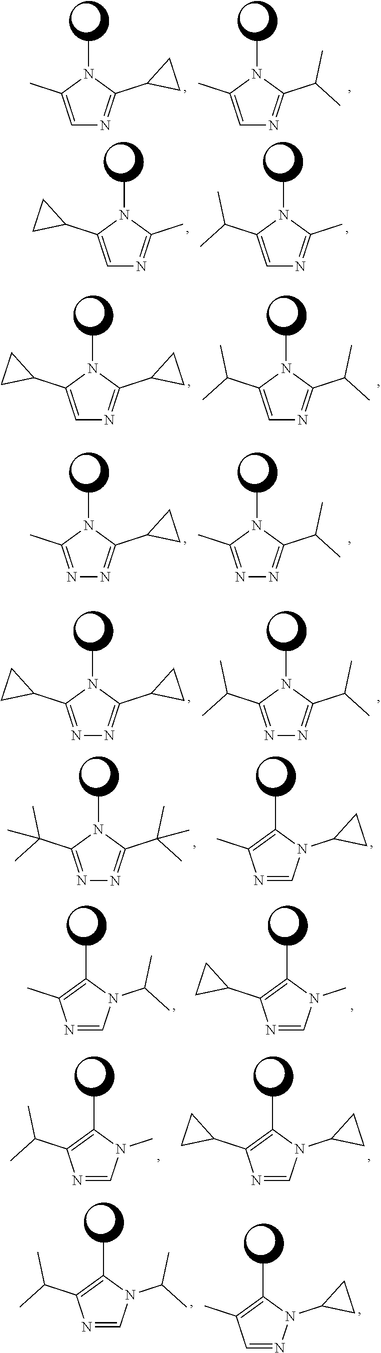

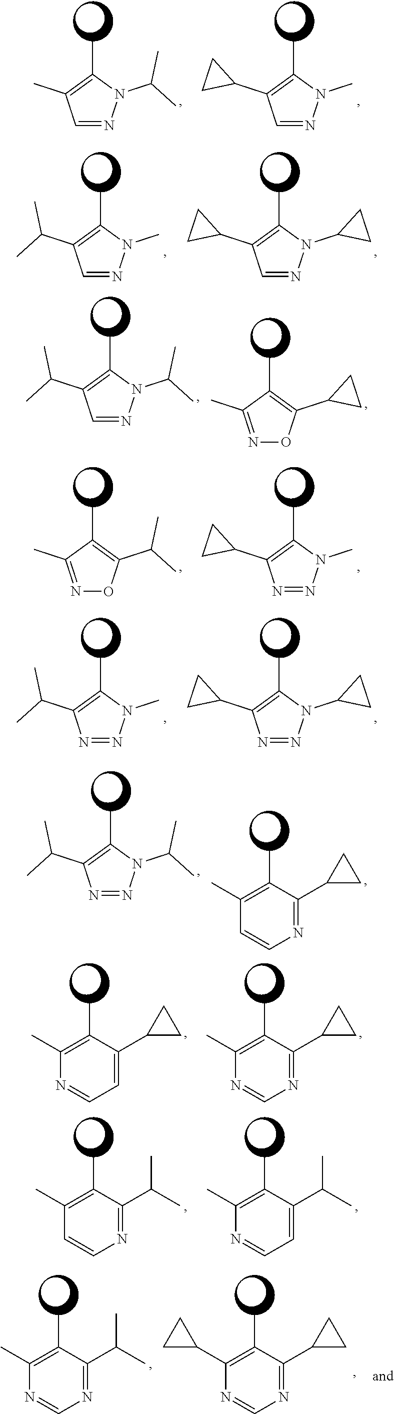

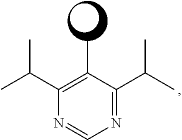

wherein the shaded circle represents the point of attachment to ring A.

In some embodiments of Formula (I), (I-A), (I-B), and/or (I-C), R¹⁰ is C₁₋₆ alkoxy.

In some embodiments of Formula (I), (I-A), (I-B), and/or (I-C), R¹⁰ is C₃₋₈ cycloalkyl optionally substituted with substituents independently selected from the group consisting of oxo, R^{b}, and R^{g}.

Variables X¹, X², X³, and X⁴

In some embodiments of Formula (I), (I-A), (I-B), and/or (I-C), two or three of X¹, X², X³, and X⁴ are N, and the others are an independently selected CR⁵.

In some embodiments of Formula (I), (I-A), (I-B), and/or (I-C), two of X¹, X², X³, and X⁴ are N, and the others are an independently selected CR⁵. In certain of these embodiments, R⁵ is H In certain of these embodiments, X¹ and X³ are N, and X² and X⁴ are an independently selected CR⁵. In certain of these embodiments, R⁵ is H.

In some embodiments of Formula (I), (I-A), (I-B), and/or (I-C), three of X¹, X², X³, and X⁴ are N and the other is CR⁵. In certain of these embodiments, R⁵ is H.

In certain of these embodiments, X¹, X³, and X⁴ are N and X² is CR⁵. In certain of these embodiments, R⁵ is H.

### Ring A, Variable n, and Variable R³

In some embodiments of Formula (I), (I-A), (I-B), and/or (I-C), ring A is phenyl.

In certain of these embodiments, ring A has the formula:

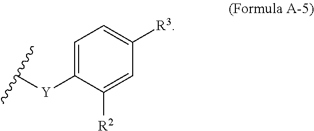

In some embodiments of Formula (I), (I-A), (I-B), and/or (I-C), n is 1.

In some embodiments of Formula (I), (I-A), (I-B), and/or (I-C), R³ is halo. For example, R³ can be fluoro.

### Variable Y

In some embodiments of Formula (I), (I-A), (I-B), and/or (I-C), Y is O.

In some embodiments of Formula (I), (I-A), (I-B), and/or (I-C), Y is S(O) or SO₂.

### Non-Limiting Combinations

In some embodiments of Formula (I), (I-A), (I-B), and/or (I-C), the compound has the formula:

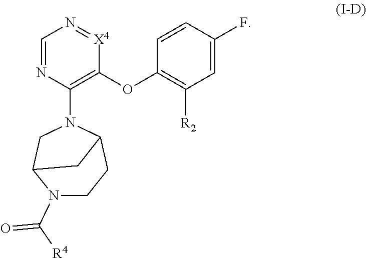

In some embodiments of Formula (I), (I-A), (I-B), (I-C), and/or (I-D), the compound has the formula:

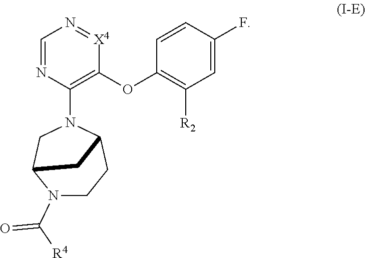

In some embodiments of Formula (I), (I-A), (I-B), (I-C), (I-D), and/or (I-E), the compound has the formula:

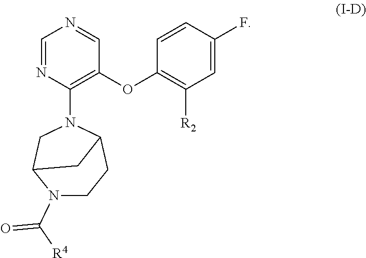

In some embodiments of Formula (I), (I-A), (I-B), and/or (I-C), the compound has the formula:

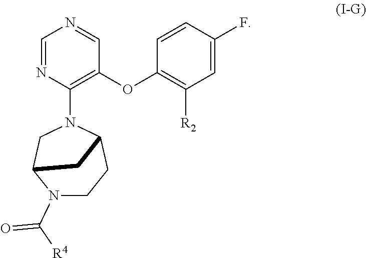

R² in Formula (I-D), (I-E), (I-F), and/or (I-G) can be as defined anywhere herein.

R⁴ in Formula (I-D), (I-E), (I-F), and/or (I-G) can be as defined anywhere herein.

R² and R⁴ in Formula (I-D), (I-E), (I-F), and/or (I-G) can be as defined anywhere herein.
[i] In some embodiments of Formula (I), (I-A), (I-B), (I-C), (I-D), (I-E), (I-F), and/or (I-G):
R⁴ is L₁-heterocyclyl of 3-12 ring atoms, wherein 1-3 ring atoms are heteroatoms, each independently selected from the group consisting of N(H), N(R^{d}), 0, and S(O)₀₋₂, and wherein the heterocyclyl is optionally substituted with 1-4 substituents independently selected from the group consisting of oxo, R^{b}, and R^{g}; and
R² is:

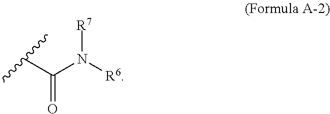

[ii] In some embodiments of Formula (I), (I-A), (I-B), (I-C), (I-D), (I-E), (I-F), and/or (I-G):
R⁴ is L₁-heterocyclyl of 3-12 ring atoms, wherein 1-3 ring atoms are heteroatoms, each independently selected from the group consisting of N(H), N(R^{d}), 0, and S(O)₀₋₂, and wherein the heterocyclyl is optionally substituted with 1-4 substituents independently selected from the group consisting of oxo, R^{b}, and R^{g}; and
R² is:

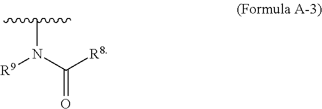

[iii] In some embodiments of Formula (I), (I-A), (I-B), (I-C), (I-D), (I-E), (I-F), and/or (I-G):
R⁴ is L₁-heterocyclyl of 3-12 ring atoms, wherein 1-3 ring atoms are heteroatoms, each independently selected from the group consisting of N(H), N(R^{d}), 0, and S(O)₀₋₂, and wherein the heterocyclyl is optionally substituted with 1-4 substituents independently selected from the group consisting of oxo, R^{b}, and R^{g}; and
R² is R¹⁰, optionally, wherein R¹⁰ is L₁-heteroaryl of 5-12 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of oxo, R^{e}, and R^{g}.

In some embodiments of combinations [i], [ii], and [iii], R⁴ is monocyclic.

In certain embodiments of combinations [i], [ii], and [iii], R⁴ is L₁-monocyclic heterocyclyl of 4-6 ring atoms, wherein 1-2 ring atoms are heteroatoms, each independently selected from the group consisting of N(H), N(R^{d}), O, and S(O)₀₋₂, which is optionally substituted with 1-4 substituents independently selected from the group consisting of oxo, R^{b}, and R^{g} (and/or an N-oxide thereor).

In certain embodiments of combinations [i], [ii], and [iii], R⁴ is L₁-monocyclic heterocyclyl of 4-6 ring atoms, wherein 1-2 ring atoms are heteroatoms, each independently selected from the group consisting of N(H), N(R^{d}), and O, which is optionally substituted with 1-4 substituents independently selected from the group consisting of oxo, R^{b}, and R^{g} (and/or an N-oxide thereor).

For example, in combinations [i], [ii], and [iii], R⁴ can be selected from the group consisting of L₁-oxetanyl, L₁-azetidinyl, L₁-thietanyl, L₁-thietanyl-1,1-dioxide, L₁-piperidinyl, L₁-piperazinyl, L₁-pyrrolidinyl, L₁-dioxanyl, L₁-morpholinyl, L₁-tetrahydrofuranyl, L₁-tetrehydrothienyl, and L₁-tetrehydrothienyl-1,1-dioxide, each of which is optionally substituted with 1-4 substituents independently selected from the group consisting of oxo, R^{b}, and R^{g}; optionally wherein R⁴ is L₁-pyrrolidinyl, which is optionally substituted with 1-4 substituents independently selected from the group consisting of oxo, R^{b}, and R^{g} (and/or an N-oxide thereor); optionally wherein R⁴ is 2-methylpyrolidin-2-yl(and/or an N-oxide thereor).

In certain of the foregoing embodiments, L₁ is a bond.

In other embodiments, L₁ is C₁-C₄ alkylene.

In some embodiments of combinations [i], [ii], and [iii], R⁴ is bicyclic.

In certain embodiments of combinations [i], [ii], and [iii], R⁴ is L₁-bicyclic heterocyclyl of 4-12 ring atoms, wherein 1-3 ring atoms are heteroatoms, each independently selected from the group consisting of N, N(H), N(R^{d}), O, and S(O)₀₋₂, and wherein the heterocyclyl is optionally substituted with 1-4 substituents independently selected from the group consisting of oxo, R^{b}, and R^{g} (and/or an N-oxide thereor).

In certain embodiments of combinations [i], [ii], and [iii], R⁴ is L₁-bicyclic heterocyclyl of 6-10 ring atoms, wherein 1-3 ring atoms are heteroatoms, each independently selected from the group co nsisting of N, N(H), N(R^{d}), O, and S(O)₀₋₂, and wherein the heterocyclyl is optionally substituted with 1-4 substituents independently selected from the group consisting of oxo, R^{b}, and R^{g} (and/or an N-oxide thereor).

In certain embodiments of combinations [i], [ii], and [iii], R⁴ is L₁-bicyclic heterocyclyl of 6-8 ring atoms, wherein 1-3 ring atoms are heteroatoms, each independently selected from the group consisting of N, N(H), N(R^{d}), O, and S(O)₀₋₂, and wherein the heterocyclyl is optionally substituted with 1-4 substituents independently selected from the group consisting of oxo, R^{b}, and R^{g} (and/or an N-oxide thereor).

In certain embodiments of combinations [i], [ii], and [iii], R⁴ is selected from the group consisting of L₁-2-azabicyclo[3.1.0]hexanyl, L₁-3-azabicyclo[3.1.0]hexanyl, L₁-2-azabicyclo[2.1.1]hexanyl, L₁-5-azabicyclo[2.1.1]hexanyl, L₁-5-azaspiro[2.4]heptanyl, L₁-3-azabicyclo[3.3.0]octanyl, L₁-3-azabicyclo[3.3.0]octanyl, L₁-hexahydro-TH-pyrrolizinyl, and L₁-4-azaspiro[2.4]heptanyl, each of which is optionally substituted with 1-4 substituents independently selected from the group consisting of oxo, R^{b}, and R^{g} (and/or an N-oxide thereor).

For example, R⁴ can be selected from the group consisting of.

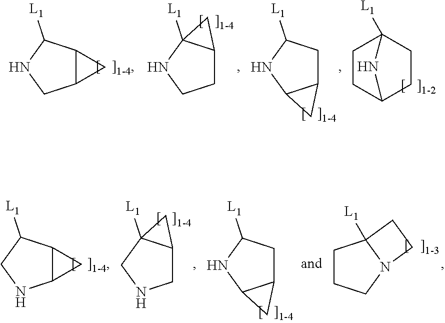

each of which is optionally substituted with 1-4 substituents independently selected from the group consisting of oxo, R^{b}, and R^{g} (and/or an N-oxide thereor).

As another example, R⁴ can be selected from the group consisting of

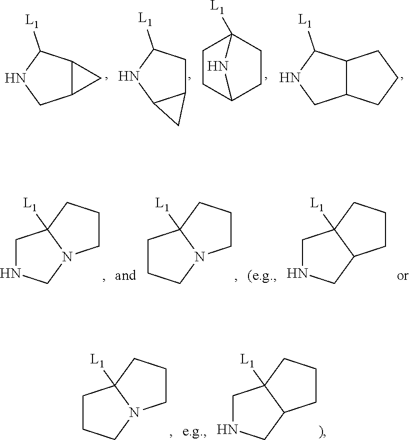

each of which is optionally substituted with 1-4 substituents independently selected from the group consisting of oxo, R^{b}, and R^{g}(and/or an N-oxide thereor).

As a further example, R⁴ can have one of the following formulas:

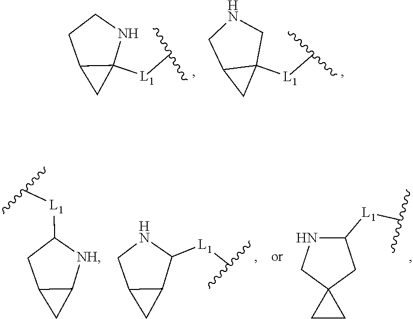

each of which is optionally substituted with 1-4 substituents independently selected from the group consisting of oxo, R^{b}, and R⁹ (and/or an N-oxide thereor).

In some embodiments of combinations [i], [ii], and [iii], each of R⁶ and R⁷ is an independently selected C₁₋₄ alkyl. For example, one of R⁶ and R⁷ is —CH₂CH₃, and the other is —CH(CH₃)₂.

In some embodiments of combinations [i], [ii], and [iii], each of R⁸ and R⁹ is an independently selected C₁₋₄ alkyl. For example, one of R⁸ and R⁹ is —CH₂CH₃, and the other is —CH(CH₃)₂.

In some embodiments of combinations [i], [ii], and [iii], R¹⁰ is selected from the group consisting of L₁-thienyl, L₁-pyridinyl, L₁-furyl, L₁-oxazolyl, L₁-oxadiazolyl, L₁-pyrrolyl, L₁-imidazolyl, L₁-triazolyl, L₁-thiodiazolyl, L₁-pyrazolyl, L₁-isoxazolyl, L₁-thiadiazolyl, L₁-pyrazinyl, L₁-pyrimidinyl, L₁-pyridazinyl, and L₁-triazinyl, each of which is optionally substituted with 1-4 substituents independently selected from the group consisting of oxo, R^{e}, and R⁹. In certain of these embodiments, L₁ is a bond.

For example, R¹⁰ can be selected from the group consisting of.

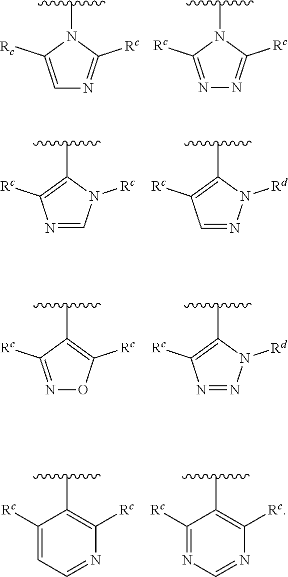

In some of the foregoing R¹⁰ embodiments, each R^{c} is, independently, C₁₋₄ alkyl, C₁₋₄ haloalkyl, or C₃₋₆ cycloalkyl.

In some of the foregoing R¹⁰ embodiments, each R^{c} is, independently, C₁₋₄ alkyl, or C₃₋₆ cycloalkyl.

In some of the foregoing R¹⁰ embodiments, each R^{c} is, independently, —CH₃, —CH(CH₃)₂, or cyclopropyl.

In some of the foregoing R¹⁰ embodiments, each R^{d} is, independently, C₁₋₄ alkyl or C₃₋₆ cycloalkyl.

In some of the foregoing R¹⁰ embodiments, each R^{c} is, independently, —CH₃ or cyclopropyl.

For example, in some of the foregoing R¹⁰ embodiments, R¹⁰ is selected from the group consisting of:

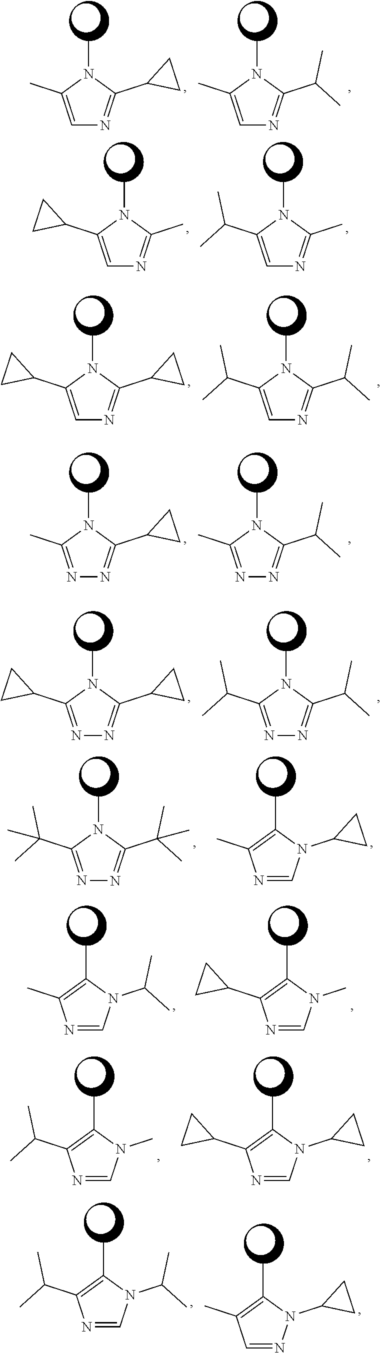

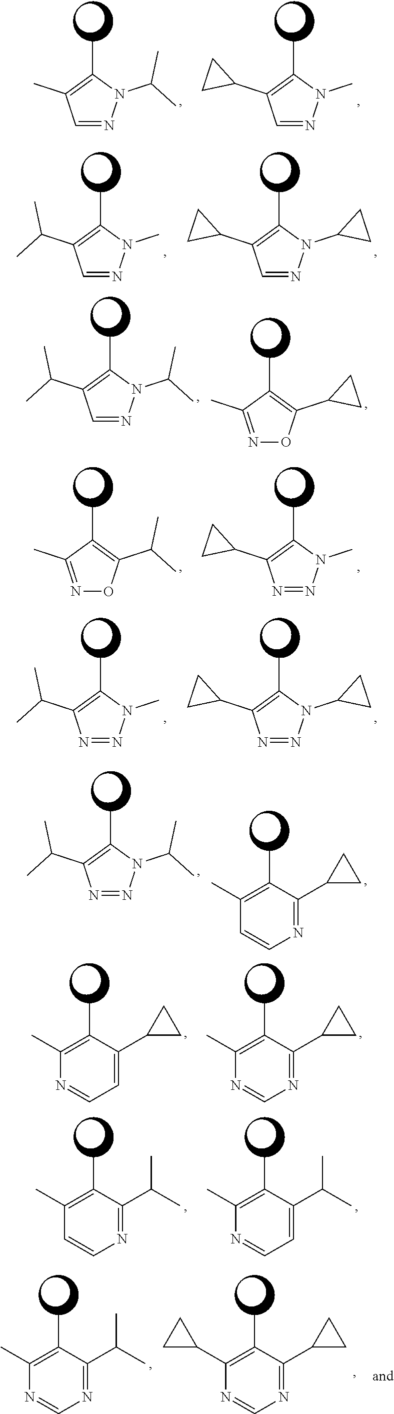

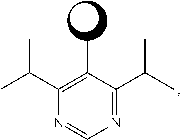

wherein the shaded circle represents the point of attachment to ring A.

The compounds of this disclosure include isotopically labeled compounds of the disclosure. An “isotopically” or “radio-labeled” compound is a compound of the disclosure where one or more atoms are replaced or substituted by an atom having an atomic mass or mass number different from the atomic mass or mass number typically found in nature (i.e., naturally occurring). Suitable radionuclides that may be incorporated in compounds of the present disclosure include but are not limited to 2H (also written as D for deuterium), 3H (also written as T for tritium), 11C, 13C, 14C, 13N, 15N, 15O, 17O, 18O, 18F, 35S, 36Cl, 82Br, 75Br, 76Br, 77Br, 123I, 124I, 125I and 131I. For example, one or more hydrogen atoms in a compound of the present disclosure can be replaced by deuterium atoms (e.g., one or more hydrogen atoms of a C₁-6 alkyl group of Formula (I) can be optionally substituted with deuterium atoms, such as —CD3 being substituted for —CH3). In some embodiments, alkyl groups of the disclosed Formulas (e.g., Formula (I)) can be perdeuterated.

### Pharmaceutical Compositions and Administration

### General

In some embodiments, the chemical entities described herein is administered as a pharmaceutical composition that includes the chemical entity and one or more pharmaceutically acceptable excipients, and optionally one or more additional therapeutic agents as described herein.

In some embodiments, the chemical entities can be administered in combination with one or more conventional pharmaceutical excipients. Pharmaceutically acceptable excipients include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens, poloxamers or other similar polymeric delivery matrices, serum proteins, such as human serum albumin, buffer substances such as phosphates, tris, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, β, and γ-cyclodextrin, or chemically modified derivatives such as hydroxyalkylcyclodextrins, including 2- and 3-hydroxypropyl-β-cyclodextrins, or other solubilized derivatives can also be used to enhance delivery of compounds described herein. Dosage forms or compositions containing a chemical entity as described herein in the range of 0.005% to 100% with the balance made up from non-toxic excipient may be prepared. The contemplated compositions may contain 0.001%-100% of a chemical entity provided herein, in one embodiment 0.1-95%, in another embodiment 75-85%, in a further embodiment 20-80%. Actual methods of preparing such dosage forms are known, or will be apparent, to those skilled in this art; for example, see Remington: The Science and Practice of Pharmacy, 22^{nd} Edition (Pharmaceutical Press, London, U K. 2012).

### Routes of Administration and Composition Components

In some embodiments, the chemical entities described herein or a pharmaceutical composition thereof can be administered to subject in need thereof by any accepted route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal.

Compositions can be formulated for parenteral administration, e.g., formulated for injection via the intravenous, intramuscular, sub-cutaneous, or even intraperitoneal routes. Typically, such compositions can be prepared as injectables, as either liquid solutions or suspensions; solid forms suitable for use to prepare solutions or suspensions upon the addition of a liquid prior to injection can also be prepared; and the preparations can be emulsified. The preparation of such formulations will be known to those of skill in the art in light of the present disclosure.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum drying and freeze-drying techniques, which yield a powder of the active ingredient, plus any additional desired ingredient from a previously sterile-filtered solution thereof.

Solid dosage forms for oral administration include capsules, tablets, pills, powders, and granules. In such solid dosage forms, the chemical entity is mixed with one or more pharmaceutically acceptable excipients, such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

In one embodiment, the compositions will take the form of a unit dosage form such as a pill or tablet and thus the composition may contain, along with a chemical entity provided herein, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In another solid dosage form, a powder, marume, solution or suspension (e.g., in propylene carbonate, vegetable oils, PEG's, poloxamer 124 or triglycerides) is encapsulated in a capsule (gelatin or cellulose base capsule). Unit dosage forms in which one or more chemical entities provided herein or additional active agents are physically separated are also contemplated; e.g., capsules with granules (or tablets in a capsule) of each drug; two-layer tablets; two-compartment gel caps, etc. Enteric coated or delayed release oral dosage forms are also contemplated.

Other physiologically acceptable compounds include wetting agents, emulsifying agents, dispersing agents or preservatives that are particularly useful for preventing the growth or action of microorganisms. Various preservatives are well known and include, for example, phenol and ascorbic acid.

Topical compositions can include ointments and creams. Ointments are semisolid preparations that are typically based on petrolatum or other petroleum derivatives. Creams containing the selected active agent are typically viscous liquid or semisolid emulsions, often either oil-in-water or water-in-oil. Cream bases are typically water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant.

The emulsifier in a cream formulation is generally a nonionic, anionic, cationic or amphoteric surfactant. As with other carriers or vehicles, an ointment base should be inert, stable, nonirritating and non-sensitizing.

In any of the foregoing embodiments, pharmaceutical compositions described herein can include one or more one or more of the following: lipids, interbilayer crosslinked multilamellar vesicles, biodegradeable poly(D,L-lactic-co-glycolic acid) [PLGA]-based or poly anhydride-based nanoparticles or microparticles, and nanoporous particle-supported lipid bilayers.

### Dosages

The dosages may be varied depending on the requirement of the patient, the severity of the condition being treated and the particular compound being employed. One skilled in the medical arts can determine the proper dosage for a particular situation. The total daily dosage may be divided and administered in portions throughout the day or by means of providing continuous delivery.

In some embodiments, the compounds described herein are administered at a dosage of from about 0.001 mg/kg to about 500 mg/kg (e.g., from about 0.001 mg/kg to about 200 mg/kg; from about 0.01 mg/kg to about 200 mg/kg; from about 0.01 mg/kg to about 150 mg/kg; from about 0.01 mg/kg to about 100 mg/kg; from about 0.01 mg/kg to about 50 mg/kg; from about 0.01 mg/kg to about 10 mg/kg; from about 0.01 mg/kg to about 5 mg/kg; from about 0.01 mg/kg to about 1 mg/kg; from about 0.01 mg/kg to about 0.5 mg/kg; from about 0.01 mg/kg to about 0.1 mg/kg; from about 0.1 mg/kg to about 200 mg/kg; from about 0.1 mg/kg to about 150 mg/kg; from about 0.1 mg/kg to about 100 mg/kg; from about 0.1 mg/kg to about 50 mg/kg; from about 0.1 mg/kg to about 10 mg/kg; from about 0.1 mg/kg to about 5 mg/kg; from about 0.1 mg/kg to about 1 mg/kg; from about 0.1 mg/kg to about 0.5 mg/kg).

### Regimens

The foregoing dosages can be administered on a daily basis (e.g., as a single dose or as two or more divided doses) or non-daily basis (e.g., every other day, every two days, every three days, once weekly, twice weeks, once every two weeks, once a month).

In some embodiments, the period of administration of a compound described herein is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In an embodiment, a therapeutic compound is administered to an individual for a period of time followed by a separate period of time.

In another embodiment, a therapeutic compound is administered for a first period and a second period following the first period, with administration stopped during the second period, followed by a third period where administration of the therapeutic compound is started and then a fourth period following the third period where administration is stopped. In an aspect of this embodiment, the period of administration of a therapeutic compound followed by a period where administration is stopped is repeated for a determined or undetermined period of time. In a further embodiment, a period of administration is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

### Methods of Treatment

This disclosure provides methods of treating a disease, condition, or disorder that is causally related to the aberrant activity of a menin-MLL interaction in a subject (e.g., a human patient), which include administering to the patient a therapeutically effective amount of any formula (I) compound described herein, or a pharmaceutically acceptable salt thereof.

The compounds described herein are useful in treating diseases associated with the menin-MLL interaction or menin-MLL fusion protein interaction. For example, diseases and conditions treatable according to the methods of the invention include cancer, such as leukaemia, and other diseases or disorders mediated by the menin-MLL interaction or menin-MLL fusion protein interaction such as diabetes.

In some embodiments, the disease, condition, or disorder is an autoimmune disease, a heteroimmune disease, a cancer, a metabolic disease, mastocytosis, osteoporosis or bone resorption disorder, or an inflammatory disease.

In some embodiments, the cancers include hematological cancer (e.g., leukaemia and lymphoma), bladder cancer, brain cancer (e.g., glioma, diffuse intrinsic pontine glioma (DIPG)), breast cancer (e.g., triple-negative breast cancer, estrogen-receptor-positive breast cancer (i.e., ER+breast cancer)), colorectal cancer, cervical cancer, gastrointestinal cancer (e.g., colorectal carcinoma, gastric cancer), genitourinary cancer, head and neck cancer, liver cancer, lung cancer, melanoma, ovarian cancer, pancreatic cancer, prostate cancer (e.g., castration resistant prostate cancer), renal cancer (e.g., renal cell carcinoma), skin cancer, thyroid cancer (e.g., papillary thyroid carcinoma), testicular cancer, sarcoma (e.g., Ewing's sarcoma), and AIDS-related cancers. In some embodiments, the cancer is associated with a rearranged MLL gene. In some embodiments, the pathophysiology of the cancer is dependent on the MLL gene. In some embodiments, the cancer is associated with mutant p53 gain-of-function.

Other examples include cardiac cancers, such as for example, sarcoma (e.g., angiosarcoma, fibrosarcoma, rhabdomyosarcoma, and liposarcoma), myxoma, rhabdomyoma, fibroma, lipoma and teratoma; lung cancers, including, for example, bronchogenic carcinoma (e.g., squamous cell, undifferentiated small cell, undifferentiated large cell, and adenocarcinoma), alveolar and bronchiolar carcinoma, bronchial adenoma, sarcoma, lymphoma, chondromatous hamartoma, mesothelioma, non-small cell lung cancer, small cell lung cancer, bronchial adenomas/carcinoids, and pleuropulmonary blastoma; gastrointestinal cancer, including, for example, cancers of the esophagus (e.g., squamous cell carcinoma, adenocarcinoma, leiomyosarcoma, and lymphoma), cancers of the stomach (e.g., carcinoma, lymphoma, and leiomyosarcoma), cancers of the pancreas (e.g., ductal adenocarcinoma, insulinoma, glucagonoma, gastrinoma, carcinoid tumors, and vipoma), cancers of the small bowel (e.g., adenocarcinoma, lymphoma, carcinoid tumors, Kaposi's sarcoma, leiomyoma, hemangioma, lipoma, neurofibroma, and fibroma), cancers of the large bowel or colon, (e.g. adenocarcinoma, tubular adenoma, villous adenoma, hamartoma, and leiomyoma), and other cancers of the digestive tract (e.g., anal cancer, anorectal cancer, appendix cancer, cancer of the anal canal, cancer of the tongue, gallbladder cancer, gastrointestinal stromal tumor (GIST), colon cancer, colorectal cancer, extrahepatic bile duct cancer, intrahepatic bile duct cancer, rectal cancer, and small intestine cancer); genitourinary tract cancers, including, for example, cancers of the kidney (e.g., adenocarcinoma, Wilm's tumor (nephroblastoma), lymphoma, and leukaemia), cancers of the bladder and urethra (e.g., squamous cell carcinoma, transitional cell carcinoma, and adenocarcinoma), cancers of the prostate (e.g., adenocarcinoma and sarcoma), cancers of the testis, (e.g., seminoma, teratoma, embryonal carcinoma, teratocarcinoma, choriocarcinoma, sarcoma, interstitial cell carcinoma, fibroma, fibroadenoma, adenomatoid tumors, and lipoma), as well as transitional cell cancer, transitional cell cancer of the renal pelvis and ureter and other urinary organs, urethral cancer, and urinary bladder cancer; liver cancers, including, for example, hepatoma (e.g., hepatocellular carcinoma), cholangiocarcinoma, hepatoblastoma, angiosarcoma, hepatocellular adenoma, and hemangioma; bone cancers, including, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochrondroma (osteocartilaginous exostoses), benign chondroma, chondroblastoma, chondromyxoibroma, osteoid osteoma and giant cell tumors; nervous system cancers, including, for example, cancers of the skull (e.g., osteoma, hemangioma, granuloma, xanthoma, and osteitis deformans); cancers of the meninges (e.g., meningioma, meningiosarcoma, and gliomatosis); cancers of the brain (e.g., astrocytoma, medulloblastoma, glioma, ependymoma, germinoma (pinealoma), glioblastoma multiforme, oligodendroglioma, schwannoma, retinoblastoma, and congenital tumors); cancers of the spinal cord (e.g., neurofibroma, meningioma, glioma, and sarcoma), and other nervous system cancers (e.g., brain stem glioma, diffuse intrinsic pontine glioma (DIPG), brain tumor, central nervous system cancer, cerebellar astrocytoma, cerebral astrocytoma/malignant glioma, childhood cerebellar astrocytoma, childhood cerebral astrocytoma, primary central nervous system lymphoma, visual pathway and hypothalamic glioma, nervous system lymphoma, supratentorial primitive neuroectodeimal tumors, pineoblastoma and supratentorial primitive neuroectodermal tumors); gynecological cancers, including, for example, cancers of the uterus (e.g., endometrial carcinoma), cancers of the cervix (e.g., cervical carcinoma, and pre tumor cervical dysplasia), cancers of the ovaries (e.g., ovarian carcinoma, including serous cystadenocarcinoma, mucinous cystadenocarcinoma, unclassified carcinoma, granulosathecal cell tumors, Sertoli Leydig cell tumors, dysgerminoma, and malignant teratoma), cancers of the vulva (e.g., squamous cell carcinoma, intraepithelial carcinoma, adenocarcinoma, fibrosarcoma, and melanoma), cancers of the vagina (e.g., clear cell carcinoma, squamous cell carcinoma, botryoid sarcoma, and embryonal rhabdomyosarcoma), and cancers of the fallopian tubes (e.g., carcinoma); other reproductive tract cancers, including, for example, endometrial cancer, endometrial uterine cancer, germ cell tumor, gestational trophoblastic tumor, gestational trophoblastic tumor glioma, ovarian epithelial cancer, ovarian germ cell tumor, ovarian low malignant potential tumor, penile cancer, vaginal cancer, vulvar cancer, extracranial germ cell tumor, extragonadal germ cell tumor, uterine cancer, uterine corpus cancer, uterine sarcoma; lymphatic and hematologic cancers, including, for example, cancers of the blood (e.g., acute myeloid leukaemia (AML), chronic myeloid leukaemia (CML), acute lymphoblastic leukaemia (ALL), chronic lymphoblastic leukaemia, chronic lymphocytic leukaemia, myeloproliferative diseases, multiple myeloma, and myelodysplastic syndrome, Hodgkin's lymphoma, non Hodgkin's lymphoma (malignant lymphoma) and Waldenstrom's macroglobulinemia), and other lymphatic or hematologic cancers including, for example, childhood leukaemia, myeloproliferative disorders (e.g., primary myelofibrosis), plasma cell neoplasm/multiple myeloma, myelodysplasia, myelodysplastic syndrome, cutaneous T-cell lymphoma, lymphoid neoplasm, AIDS-related lymphoma, thymoma, thymoma and thymic carcinoma, mycosis fungoides, and Sezary Syndrome; skin cancers, including, for example, malignant melanoma, basal cell carcinoma, squamous cell carcinoma, Kaposi's sarcoma, moles dysplastic nevi, lipoma, angioma, dermatofibroma, keloids, psoriasis, merkel cell carcinoma, merkel cell skin carcinoma, melanoma, and carcinoid tumor: adrenal gland cancers, including, for example, neuroblastoma; other cancers associated with the endocrine system including, for example, adrenocortical carcinoma, multiple endocrine neoplasia (e.g., multiple endocrine neoplasia type I), multiple endocrine neoplasia syndrome, parathyroid cancer, pituitary tumor, pheochromocytoma, islet cell pancreatic cancer, and islet cell tumors): connective tissue cancer (e.g., bone cancer, bone and joint cancer, osteosarcoma and malignant fibrous histiocytoma); cancer associated with the head, neck, and mouth (e.g., head and neck cancer, paranasal sinus and nasal cavity cancer, metastatic squamous neck cancer, mouth cancer, throat cancer, esophageal cancer, laryngeal cancer, pharyngeal cancer, hypopharyngeal cancer, lip and oral cavity cancer, nasopharyngeal cancer, oral cancer, oropharyngeal cancer, and salivary gland cancer); and cancer associated with the eye (e.g., ocular cancer, intraocular melanoma). In some embodiments, the cancer is Ewing's sarcoma.

In some embodiments, the cancer is a hematological cancer such as leukaemia or lymphoma. Example leukaemia and lymphomas treatable by the compounds of the invention include mixed lineage leukaemia (MLL), MLL-related leukaemia, MLL-associated leukaemia, MLL-positive leukaemia, MLL-induced leukaemia, rearranged mixed lineage leukaemia (MLL-r), leukaemia associated with a MLL rearrangement or a rearrangement of the MLL gene, acute leukaemia, chronic leukaemia, indolent leukaemia, lymphoblastic leukaemia, lymphocytic leukaemia, myeloid leukaemia, myelogenous leukaemia, childhood leukaemia, acute lymphocytic leukaemia (ALL) (also referred to as acute lymphoblastic leukaemia or acute lymphoid leukaemia), acute myeloid leukaemia (AML) (also referred to as acute myelogenous leukaemia or acute myeloblastic leukaemia), acute granulocytic leukaemia, acute nonlymphocytic leukaemia, chronic lymphocytic leukaemia (CLL) (also referred to as chronic lymphoblastic leukaemia), chronic myelogenous leukaemia (CML) (also referred to as chronic myeloid leukaemia), therapy related leukaemia, myelodysplastic syndrome (MDS), myeloproliferative disease (MPD) (such as primary myelofibrosis (PMF)), myeloproliferative neoplasia (MPN), plasma cell neoplasm, multiple myeloma, myelodysplasia, cutaneous T-cell lymphoma, lymphoid neoplasm, AIDS-related lymphoma, thymoma, thymic carcinoma, mycosis fuingoides, Alibert-Bazin syndrome, granuloma fungoides, Sezary Syndrome, hairy cell leukaemia, T-cell prolymphocytic leukaemia (T-PLL), large granular lymphocytic leukaemia, meningeal leukaemia, leukemic leptomeningitis, leukemic meningitis, multiple myeloma, Hodgkin's lymphoma, non Hodgkin's lymphoma (malignant lymphoma), and Waldenstrom's macroglobulinemia. In some embodiments, the acute myeloid leukaemia (AIL) is abstract nucleophosmin (NPM1)-mutated acute myeloid leukaemia (i.e., NPM1mut acute myloid leukaemia).

In particular embodiments, the compounds described herein are used to treat leukaemia associated with a MLL rearrangement, acute lymphocytic leukaemia associated with a MLL rearrangement, acute lymphoblastic leukaemia associated with a MLL rearrangement, acute lymphoid leukaemia associated wdith a MLL rearrangement, acute myeloid leukaemia associated with a MLL rearrangement, acute myelogenous leukaemia associated with a MLL rearrangement, or acute myeloblastic leukaemia associated with a MLL rearrangement. As used herein, “MLL rearrangement” means a rearrangement of the MLL gene.

In some embodiments, diseases and conditions also include insulin resistance, pre-diabetes, diabetes (e.g., Type 2 diabetes or Type 1 diabetes), and risk of diabetes. In some embodiments, diseases and conditions treatable with compounds of the invention include hyperglycemia. In some embodiments, the hyperglycemia is associated with diabetes, such as Type 2 diabetes. In some embodiments, compounds described herein are used to treat loss of response to other anti-diabetic agents and/or reduced beta cell function in a patient or subject. In some embodiments, compounds of the invention are used to restore response to other anti-diabetic agents and/or to restore beta cell function, and/or stimulate beta cell proliferation, and/or to reduce the need for insulin in a patient or subject. In some embodiments, compounds of the invention are used to reduce insulin resistance, reduce the risk of diabetes, or reduce increases in blood glucose caused by a statin in a subject taking a statin. In some embodiments, compounds of the invention are used to treat diabetes in a subject taking a statin or to prevent diabetes in a subject taking a statin. Methods of the invention include decreasing, reducing, inhibiting, suppressing, limiting or controlling in the patient elevated blood glucose levels. In further aspects, methods of the invention include increasing, stimulating, enhancing, promoting, inducing or activating in the subject insulin sensitivity. Statins include, but are not limited to atorvastatin, cerivastatin, fluvastatin, lovastatin, mevastatin, pitavastatin, pravastatin, rousuvastatin and simvastatin.

### Combinations

The disclosure also features combination therapy for treating a disease or a disorder described herein. In some embodiments, the combination therapy comprises administering at least one compound described herein in combination with one or more other pharmaceutically active agents for treating cancer or other disorders mediated by menin/MLL. In some embodiments, the combination therapy comprises administering at least one compound of the present invention in combination with one or more other pharmaceutically active agents, such as for the treatment of cancer. For example, in the field of medical oncology it is normal practice to use a combination of different forms of treatment to treat each subject with cancer. In medical oncology the other component(s) of such conjoint treatment or therapy in addition to compositions provided herein may be, for example, surgery, radiotherapy, and chemotherapeutic agents. For example, a surgery may be open surgery or minimally invasive surgery. Compounds of Formula (I), or pharmaceutically acceptable salts or solvates thereof, therefore may also be useful as adjuvants to cancer treatment, that is, they can be used in combination with one or more additional therapies or therapeutic agents, for example, a chemotherapeutic agent that works by the same or by a different mechanism of action. In some embodiments, a compound of Formula (I), or a pharmaceutically acceptable salt thereof, can be used prior to administration of an additional therapeutic agent or additional therapy. For example, a subject in need thereof can be administered one or more doses of a compound of Formula (I), or a pharmaceutically acceptable salt thereof, for a period of time and then undergo at least partial resection of the tumor. In some embodiments, the treatment with one or more doses of a compound of Formula (I), or a pharmaceutically acceptable salt thereof, reduces the size of the tumor (e.g., the tumor burden) prior to the at least partial resection of the tumor. In some embodiments, a subject in need thereof can be administered one or more doses of a compound of Formula (I), or a pharmaceutically acceptable salt thereof, for a period of time and under one or more rounds of radiation therapy. In some embodiments, the treatment with one or more doses of a compound of Formula (I), or a pharmaceutically acceptable salt thereof, reduces the size of the tumor (e.g., the tumor burden) prior or subsequent to the one or more rounds of radiation therapy.

One or more additional pharmaceutical agents, for example, chemotherapeutics, anti-inflammatory agents, steroids, immunosuppressants, immune-oncology agents, metabolic enzyme inhibitors, chemokine receptor inhibitors, phosphatase inhibitors, and statins as well as targeted therapies can be used in combination with the compounds of the present disclosure for treatment of the-associated diseases, disorders or conditions described herein. The one or more additional pharmaceutical agents can be administered to a patient simultaneously or sequentially.

### Compound Preparation

The compounds disclosed herein can be prepared in a variety of ways using commercially available starting materials, compounds known in the literature, or from readily prepared intermediates, by employing standard synthetic methods and procedures either known to those skilled in the art, or in light of the teachings herein. The synthesis of the compounds disclosed herein can be achieved by generally following Scheme 1, with modification for specific desired substituents.

Standard synthetic methods and procedures for the preparation of organic molecules and functional group transformations and manipulations can be obtained from the relevant scientific literature or from standard textbooks in the field. Although not limited to any one or several sources, classic texts such as R. Larock, Comprehensive Organic Transformations, VCH Publishers (1989); L. Fieser and M. Fieser, Fieser and Fieser's Reagents for Organic Synthesis, John Wiley and Sons (1994); Smith, M. B., March, J., March's Advanced Organic Chemistry: Reactions, Mechanisms, and Structure, 5th edition, John Wiley & Sons: New York, 2001; and Greene, T. W., Wuts, P. G. M., Protective Groups in Organic Synthesis, 3rd edition, John Wiley & Sons: New York, 1999, are useful and recognized reference textbooks of organic synthesis known to those in the art. The following descriptions of synthetic methods are designed to illustrate, but not to limit, general procedures for the preparation of compounds of the present disclosure.

The synthetic processes disclosed herein can tolerate a wide variety of functional groups; therefore, various substituted starting materials can be used. The processes generally provide the desired final compound at or near the end of the overall process, although it may be desirable in certain instances to further convert the compound to a pharmaceutically acceptable salt thereof.

### EXAMPLES

The starting materials used for the synthesis were synthesized according to known literature procedures or obtained from commercial sources, such as, but not limited to, Sigma-Aldrich, Fluka, Acros Organics, AlfaAesar, VWR Scientific, and the like. Nuclear Magnetic Resonance (NMR) analysis was conducted using either a 400 or 600 MHz spectrometer with an appropriate deuterated solvent. NMR chemical shift (6) is expressed in units of parts per million (ppm). General methods for the preparation of compounds can be modified using appropriate reagents and conditions for the introduction of the various moieties found in the structures as provided herein.

While preferred embodiments of the present invention have been shown and described herein, it will be obvious to those skilled in the art that such embodiments are provided by way of example only. Numerous variations, changes, and substitutions will now occur to those skilled in the art without departing from the invention. Various alternatives to the embodiments of the invention described herein may be employed in practicing the invention. It is intended that the following claims define the scope of the invention and that methods and structures within the scope of these claims and their equivalents be covered thereby.

Standard abbreviations and acronyms as defined in Journal of Organic Chemistry's Author's Guideline, and in Hans Reich's Collection. Organic Acronyms are used herein.

Several general methods relating to the synthesis of the compounds are herein disclosed. These methods by no means limit how these compounds can be prepared by one of ordinary skill in the art.

### General Procedure A1: Amide Bond Formation Using HATU, DIEA/NEt₃ in DMF/DMAc

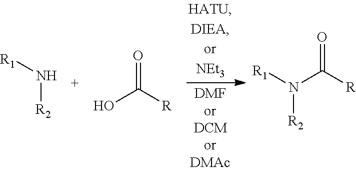

A solution of a primary or secondary amine (1 equiv), a carboxylic acid (1-1.5 equiv), HATU (1.2-1.5 equiv), DIEA or NEt₃ (2-10 equiv) in DMF or DCM or DMAc (6-10 mL/mmol) was stirred at room temperature for 1-16 h under nitrogen. The resulting mixture was concentrated under reduced pressure. The crude residue was generally purified directly according to the indicated method.

### General Procedure A2: Amide Bond Formation

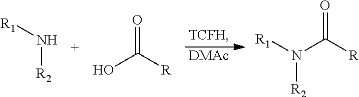

A solution of a primary or secondary amine (1.0 equiv) and a carboxylic acid (1.0 equiv), TCFH (1.5 equiv), 1-methyl-1H-imidazole (9.0 equiv) in MeCN (3 mL) was stirred at room temperature for 16 h. The resulting mixture was diluted with water (5 mL), the resulting mixture was extracted with EtOAc (3×10 mL) and the combined organic layers were washed with brine (4 mL), dried over Na₂SO₄, filtered and the filtrate was purified by Prep-HPLC to give the indicated product.

### General Procedure A3: Amide Bond Formation Using EDCI/HOBt

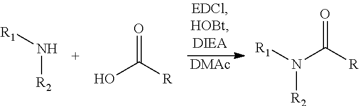

A solution of a primary or secondary amine (1 equiv) and a carboxylic acid (1.2 equiv), EDCI (1.2 equiv), HOBT (0.3 equiv), DIEA (3.0 equiv), in DMAc (3 mL) was stirred at room temperature for 5 h. After filtration, the filtrate was purified by Prep-HPLC to give the indicated product.

### General Procedure A4: Amide Bond Formation Using T₃P

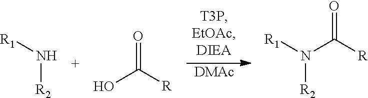

A solution of a primary or secondary amine (1 equiv) and a carboxylic acid, T₃P in EtOAc (1.2 equiv, 50%), DIEA (3 equiv) in dimethylacetamide (3 mL) was stirred at room temperature for 5 h. After filtration, the filtrate was purified by Prep-HPLC to give the indicated product.

### General Procedure A5: Amide Bond Formation Using PyBOP

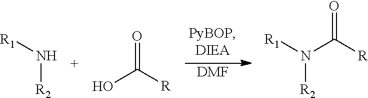

A solution of a primary or secondary amine (1.0-1.1 equiv) and a carboxylic acid (1.0-1.3 equiv.), PyBOP (1.2-1.3 equiv), DIEA (2.0-3.0 equiv) in DMF (6.5-9.5 mL/mmol) was stirred at room temperature or 80° C. for 2-16 h. After filtration, the filtrate was purified by Prep-HPLC or RPFC to give the indicated product.

### General Procedure B1: Boc Deprotection Using TFA in DCM

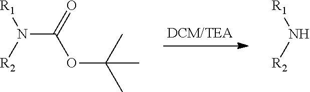

A solution of the Boc-protected analogue (1 equiv) in DCM/TFA (4:1, 5 mL/mmol) was stirred at room temperature for 1-18 h under nitrogen. The resulting mixture was concentrated under reduced pressure and the crude product was generally purified directly according to the indicated method.

### General Procedure B2: Boc Deprotection Using HCl in Dioxane/EtOAc/DCM

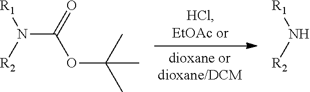

A solution of the Boc-protected analogue (1 equiv) in 4 M HCl in 1,4-dioxane or 1,4-dioxane/DCM (1:1) or EtOAc (25 mL/mmol) was stirred at room temperature for 1-18 h under nitrogen. The resulting mixture was concentrated under reduced pressure and the crude product was generally purified directly according to the indicated method.

### General Procedure B3: Boc Deprotection Using N,O-Bis(Trimethylsilyl)Acetamide in DCM

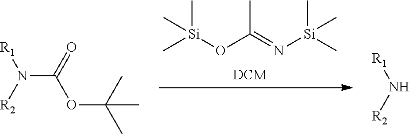

A mixture of Boc-protected analogue (25-40 mg, 1 equiv), N,O-bis(trimethylsilyl)acetamide (1.0 equiv) in DCM (3-5 mL) was stirred at room temperature for 1-18 h. The reaction was quenched with MeOH. The resulting mixture was concentrated under reduced pressure and the crude product was generally purified directly according to the indicated method.

### General Procedure B4: Boc Deprotection Using AlCl₃ in HFIP

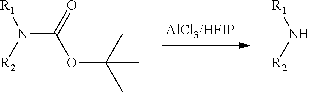

AlCl₃ (3 equiv) was added in portions to a stirred mixture of the Boc-protected analogue (1 equiv) in HFIP (0.5-1 mL/mmol), and the mixture was stirred at 0° C. under nitrogen. The resulting mixture was allowed to stir at room temperature for 1-6 h, after which it was quenched with sat. aq. NaHCO₃. The crude product was generally purified directly according to the indicated method.

### General Procedure C1: Phenol Ether Coupling

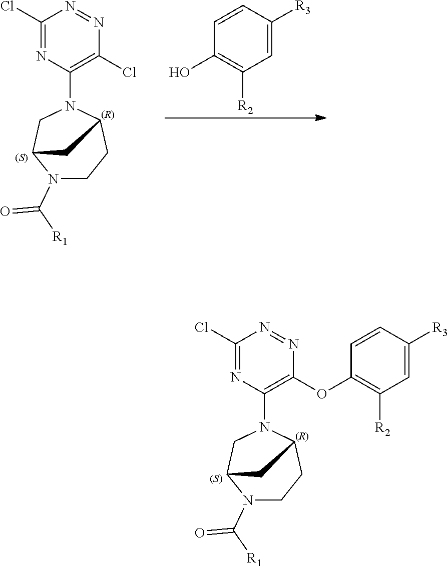

A solution of a substituted 3,6-dichlorotriazine (1.0 equiv), a phenol (0.8-2 equiv) and DBU (2 equiv) in THF (5 mL/mmol) was stirred at room temperature to 25-80° C. for 2-16 h under nitrogen, after which the mixture was allowed to cool down to room temperature. The resulting reaction mixture was generally purified according to the indicated method.

### General Procedure D1: Hydrogenolysis (De-Chlorination) of 3-Chlorotriazines Using NaBH₄

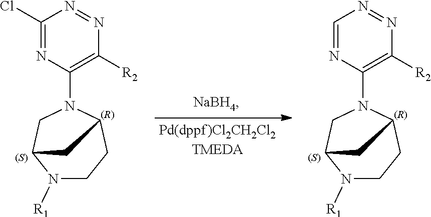

A solution of substituted 3 chlorotriazine (1.0 equiv), Pd(dppf)Cl₂CH₂Cl₂ (0.1-0.2 equiv), TMEDA (2-3 equiv) and NaBH₄ (2-3 equiv) in THF (8 mL/mmol) was stirred at room temperature for 2-16 h under nitrogen. The reaction mixture was generally purified according to the indicated method.

### General Procedure D2: Hydrogenolysis of 3-Chlorotriazines Using Pd/C

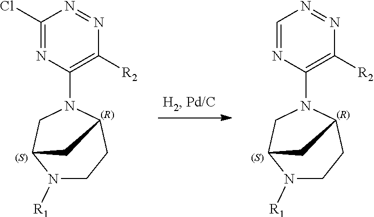

A solution of substituted 3-chlorotriazine (1.0 equiv) and Pd/C (10% wt, wet) in MeOH (22 mL/mmol) was stirred at room temperature under a hydrogen atmosphere for 16 h. The resulting mixture was filtered through a Celite pad and concentrated under reduced pressure. The reaction mixture was generally purified according to the indicated method.

### General Procedure E1: Formation of 4-Chloropyrimdines

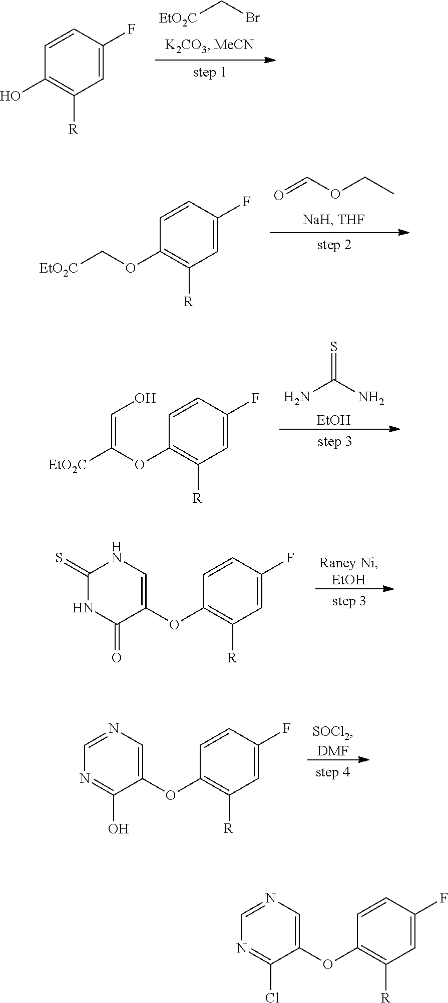

### Step 1: Synthesis of a Substituted ethyl 4-fluorophenoxyacetate

A solution of a 2-substituted 4-fluorophenol (1.0 equiv), ethyl 2-bromoacetate (1.1 equiv), and K₂CO₃ (3.0 equiv) in MeCN (2-2.5 mL/mmol) was stirred at 80° C. for 2 h under nitrogen. The resulting reaction mixture was generally purified according to the indicated method.

### Step 2: Synthesis of a Substituted ethyl (Z)-2-(4-fluorophenoxy)-3-hydroxyacrylate

To a stirred mixture of an ethyl 4-fluorophenoxyacetate (1.0 equiv) and NaH (2.0 equiv) in THF (4-5 mL/mmol) was added ethyl formate (3.0 equiv) in portions at room temperature under nitrogen. The resulting mixture was stirred at 35° C. for 1 h under nitrogen, and then quenched with EtOH at room temperature. The resulting mixture was concentrated under reduced pressure, affording crude material, which was used in the next step without further purification.

### Step 3: Synthesis of a Substituted 5-(4-fluorophenoxy)-2-thioxo-2,3-dihydropyrimidin-4(1H)-one

A mixture of an ethyl (Z)-2-(4-fluorophenoxy)-3-hydroxyacrylate (1 equiv) and thiourea (1.0 equiv) in EtOH (4-5 mL/mmol) was stirred at 80-90° C. for 16 h under nitrogen. After evaporation, the residue was purified by reversed-phase flash chromatography.

### Step 4: Synthesis of a Substituted 5-(4-fluorophenoxy)pyrimidin-4-ol

A mixture of a substituted 5-(4-fluorophenoxy)-2-thioxo-2,3-dihydropyrimidin-4(1H)-one (1.0 equiv) and Raney Ni (2.0 equiv) in EtOH (4-5 mL/mmol) was stirred at 90° C. for 1 h under nitrogen. The resulting mixture was filtered, the precipitate washed with EtOH, and the filtrate was concentrated under reduced pressure to afford crude material, which was used in the next step without further purification.

### Step 5: Synthesis of 4-chloro-5-(2-(2-cyclopropyl-5-methyl-1H-imidazol-1-yl)-4-fluorophenoxy)pyrimidine

A solution of a substituted 5-(4-fluorophenoxy)pyrimidin-4-ol (1.0 equiv) in DMF (0.2 mL/mmol) and SOCl₂ (2 mL/mmol) was stirred at 80° C. for 30 min under nitrogen. The resulting mixture was concentrated under reduced pressure, and then quenched with sat. NaHCO₃ at 0° C. The resulting residue was usually extracted with EtOAc, and the combined organic layers washed with water, and dried over Na₂SO₄. The crude 4-chloropyrimidine could be used crude in the next step, or purified as indicated.

### General Procedure F1: Synthesis of 4-fluoro-2-(5-methyl-1H-imidazol-1-yl)phenols

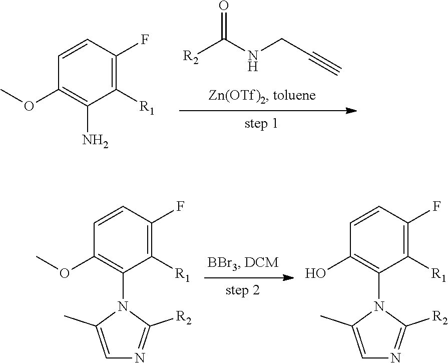

### Step 1: Synthesis of a 2-substituted 1-(5-fluoro-2-methoxyphenyl)-5-methyl-1H-imidazole

A solution of 5-fluoro-2-methoxyaniline (1.0 equiv), a substituted N-(prop-2-yn-1-yl)acetamide (1.0 equiv), Zn(OTf)₂ (0.3 equiv) in toluene (0.5-1 mL/mmol) was stirred at 90° C. overnight under nitrogen. The resulting mixture was concentrated under reduced pressure and purified by reversed-phase flash chromatography as indicated to afford the corresponding 2-substituted 1-(5-fluoro-2-methoxyphenyl)-5-methyl-1H-imidazole.

### Step 2: Synthesis of a substituted 4-fluoro-2-(5-methyl-1H-imidazol-1-yl)phenol

BBr₃ (1 M in DCM, 3.0 equiv) was added to a stirred solution of a 2-substituted 1-(5-fluoro-2-methoxyphenyl)-5-methyl-1H-imidazole (1.0 equiv) in DCM (1-2 mL/mmol), in portions at room temperature under nitrogen. The resulting mixture was allowed to stir at room temperature for 16 h, and then was quenched with MeOH at 0° C. The resulting mixture was concentrated under reduced pressure, neutralized to pH 7 with sat. NaHCO₃, and extracted with EtOAc. The combined organic layers were washed with brine, dried over Na₂SO₄, and the filtrate was concentrated under reduced pressure. The residue was purified as indicated.

### General Procedure G1: S_{N}Ar on 4-chloropyrimidines

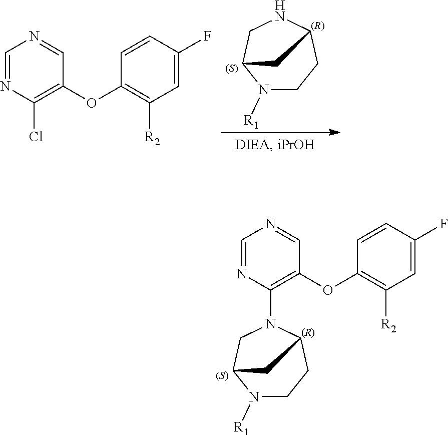

A mixture of a 4-chloropyrimidine (1 equiv), a 2,6-diazabicyclo[3.2.1]octane (0.8-1.5 equiv), and DIEA (2-3 equiv) in iPrOH (2-3 mL/mmol) was stirred at 80-90° C. under nitrogen for 1-6 h. The resulting reaction mixture was generally purified according to the indicated method.

### General Procedure H1: Reductive Aminations

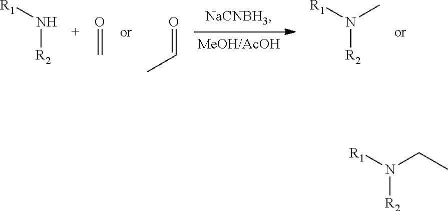

A solution of a primary or secondary amine (1 equiv), sodium cyanoborohydride (3-6 equiv), and either formaldehyde solution or acetaldehyde (3-7 equiv) in MeOH (10-20 mL/mmol) was stirred at room temperature for 1-18 h under nitrogen. The resulting reaction mixture was generally purified according to the indicated method.

General procedure I1: Synthesis of α-methylated prolines

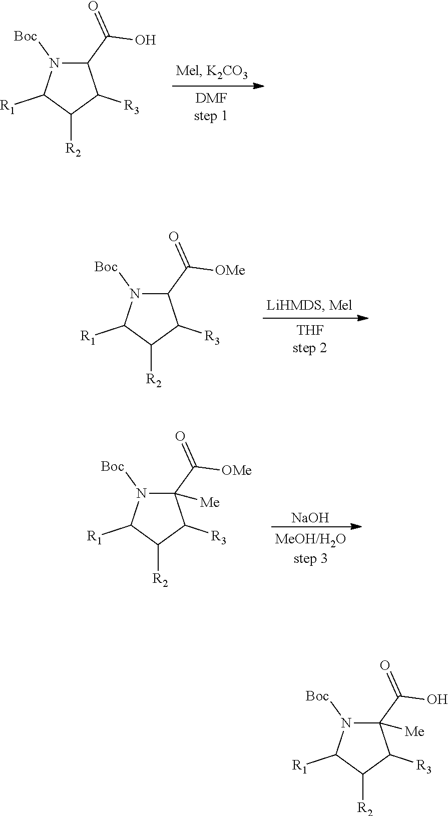

### Step 1: Synthesis of proline methyl esters

A solution of proline derivative (1.0 equiv), K₂CO₃ (3 equiv), and MeI (1.5-3.0 equiv) in DMF or THF (1-6 mL/mmol) was stirred at room temperature (DMF) or 60° C. (THF) for 1-16 h under nitrogen. The resulting mixture was filtered then diluted with water. The aqueous phase was extracted with EtOAc then the combined organic extracts were washed with brine, dried over Na₂SO₄ then concentrated under reduced pressure. The crude product was generally purified according to the indicated method.

### Step 2: α-Methylation of Proline Derivatives

To a stirred solution of proline methyl ester (1.0 equiv) in THF (2-8 mL/mmol) at −20° C. under nitrogen was added portionwise/dropwise LiHMDS (solid or 1.0 M THF solution, 1.5-2.0 equiv). The resulting mixture was stirred at −20° C. or −78° C. for 1-2 h, then MeI (1.5-2.0 equiv) was added dropwise. The resulting mixture was warmed to room temperature and stirred 2-16 h. The reaction was quenched with sat. aq. NH₄Cl at 0° C., then the aqueous phase was extracted with EtOAc. The combined organic extracts were washed with brine, dried over Na₂SO₄ then concentrated under reduced pressure. The crude product was generally purified according to the indicated method.

### Step 3: Hydrolysis of Proline Derivatives

To a stirred solution of α-methylated proline derivative (1.0 equiv) in MeOH/H₂O (1:1, 2.5-5.2 mL/mmol) was added NaOH (5-26 equiv). The resulting mixture was stirred at 50° C. for 4-16 h, then the mixture was acidified to pH 5-7 with conc. HCl. The crude product was generally purified according to the indicated method.

### Purification Methods

| Purification | | | | |
|---|---|---|---|---|
| Method | Type | Column | Mobile Phase | Detection |
| RPFC1 | Reversed-phase | C18 silica gel | MeCN in Water | 254 nm |
| | flash | | (10 mM NH₄HCO₃) | |
| | chromatography | | | |
| RPFC2 | Reversed-phase | C18 silica gel | MeCN in Water | 254 nm |
| | flash | | (0.1% FA) | |
| | chromatography | | | |
| RPFC3 | Reversed-phase | C18 silica gel | MeCN in Water | 254 nm/ |
| | flash | | | 220 nm |
| | chromatography | | | |
| RPFC4 | Reversed-phase | C18 silica gel | MeCN in Water | 254 nm |
| | flash | | (0.1% NH₃•H₂O) | |
| | chromatography | | | |
| RPFC5 | Reversed-phase | C18 silica gel | MeCN in Water | 254 nm |
| | flash | | (0.1% TFA) | |
| | chromatography | | | |
| Prep-HPLC1 | Prep-HPLC | XBridge Prep | A: Water (10 mM | 254 nm/ |
| | | OBD C18, | NH₄HCO₃ + 0.05% NH₃H₂O); | 220 nm |
| | | 30*150 mm, | B: ACN; | |
| | | 5 μm | Flow rate: 60 mL/min | |
| Prep-HPLC2 | Prep-HPLC | XBridge Prep | A: Water (10 mM | 254 nm/ |
| | | OBD C18 | NH₄HCO₃ + 0.05% NH₃H₂O); | 220 nm |
| | | Column, | B: MeOH; | |
| | | 19*250 mm, | Flow rate: 25 mL/min | |
| | | 5 μm | | |
| Prep-HPLC3 | Prep-HPLC | Welch | A: Water (10 mmol | 254 nm/ |
| | | Xtimate C18 | NH₄HCO₃); | 220 nm. |
| | | 30*150 mm, | B: ACN; | |
| | | 10 μm | Flow rate: 35 mL/min | |
| Prep-HPLC4 | Prep-HPLC | Welch | A: Water (0.1% FA); | 254 nm/ |
| | | Xtimate C18 | B: ACN; | 220 nm |
| | | 30*150 mm, | Flow rate: 35 mL/min | |
| | | 10 μm | | |
| Prep-HPLC5 | Prep-HPLC | Xselect CSH | A: Water (0.1% FA); | 254 nm/ |
| | | C18 OBD | B: ACN; | 220 nm |
| | | 19*250 mm | Flow rate: 30 mL/min | |
| | | 5 μm | | |
| Prep-HPLC6 | Prep-HPLC | Xselect CSH | A: Water (0.1% FA); | 254 nm/ |
| | | C18 OBD | B: ACN; | 220 nm |
| | | 30*150 mm | Flow rate: 60 mL/min | |
| | | 5 μm | | |
| Prep-HPLC7 | Prep-HPLC | Xbridge BEH | A: Water (10 mM | 254 nm/ |
| | | Shield RP18, | NH₄HCO₃ + 0.05% NH₃H₂O); | 220 nm |
| | | 19*250 mm | B: MeOH; | |
| | | 5 μm | Flow rate: 25 mL/min | |
| Prep-HPLC8 | Prep-HPLC | Xbridge BEH | A: Water (10 mM | 254 nm/ |
| | | Shield RP18, | NH₄HCO₃ + 0.05% NH₃H₂O); | 220 nm |
| | | 30*150 mm | B: MeOH; | |
| | | 5 μm | Flow rate: 25 mL/min | |
| Prep-HPLC9 | Prep-HPLC | XBridge Prep | A: Water (0.1% TFA); | 254 nm/ |
| | | OBD C18, | B: ACN; | 220 nm |
| | | 19*250 mm, | Flow rate: 30 mL/min | |
| | | 5 μm | | |
| Prep-HPLC10 | Prep-HPLC | YMC-Actus | A: Water (0.1% FA); | 254 nm/ |
| | | Triart C18, | B: ACN; | 220 nm |
| | | 19*250 mm, | Flow rate: 25 mL/min | |
| | | 5 μm | | |
| Prep-HPLC11 | Prep-HPLC | Xselect CSH | A: Water (0.05% TFA); | 254 nm/ |
| | | C18 OBD | B: ACN; | 220 nm |
| | | 30*150 mm | Flow rate: 60 mL/min | |
| | | 5 μm | | |
| Prep-HPLC12 | Prep-HPLC | Welch | A: Water (10 mmol | 254 nm/ |
| | | Xtimate C18 | NH₄HCO₃) | 220 nm |
| | | 30*150 mm, | B: ACN | |
| | | 10 μm | Flow rate: 35 mL/min | |
| Prep-HPLC13 | Prep-HPLC | Welch | A: Water (0.1% Formic | 254 nm/ |
| | | Xtimate C18 | Acid) | 220 nm |
| | | 30*150 mm, | B: ACN | |
| | | 10 μm | Flow rate: 35 mL/min | |
| Prep-HPLC14 | Prep-HPLC | Xbridge BEH | A: Water (10 mM | 254 nm/ |
| | | Shield RP18, | NH₄HCO₃ + 0.05% NH₃H₂O); | 220 nm |
| | | 30*150 mm | B: ACN; | |
| | | 5 μm | Flow rate: 60 mL/min | |
| Prep-HPLC15 | Prep-HPLC | Xselect CSH | A: Water (0.05% TFA); | 254 nm/ |
| | | C18 OBD | B: ACN; | 220 nm |
| | | 19*250 mm | Flow rate: 60 mL/min | |
| | | 5 μm | | |
| Prep-HPLC16 | Prep-HPLC | Xselect CSH | A: Water (0.1% FA); | 254 nm/ |
| | | C18 OBD | B: ACN; | 220 nm |
| | | 30*150 mm | Flow rate: 60 mL/min | |
| | | 5 μm | | |
| Prep-HPLC17 | Prep-HPLC | Poroshell 120 | A: Water (10 mM | 254 nm/ |
| | | 4 HPH-C18 | NH₄HCO₃ + 0.05% NH₃H₂O); | 220 nm |
| | | 21.2*150 mm | B: ACN; | |
| | | 5 μm | Flow rate: 25 mL/min | |
| Prep-HPLC18 | Prep-HPLC | YMC-Actus | A: Water (10 mM | 254 nm/ |
| | | Triart C18, | NH₄HCO₃ + 0.05% NH₃H₂O); | 220 nm |
| | | 20*250 mm, | B: ACN; | |
| | | 5 μm | Flow rate: 25 mL/min | |
| Prep-HPLC19 | Prep-HPLC | YMC-Actus | A: Water (0.1% FA); | 254 nm/ |
| | | Triart C18, | B: ACN; | 220 nm |
| | | 30*150 mm, | Flow rate: 25 mL/min | |
| | | 5 μm | | |
| Prep-HPLC20 | Prep-HPLC | XBridge Prep | A: Water (10 mM | 254 nm/ |
| | | OBD C18 | NH₄HCO₃ + 0.05% NH₃H₂O); | 220 nm |
| | | Column, | B: ACN; | |
| | | 19*250 mm, | Flow rate: 30 mL/min | |
| | | 5 μm | | |
| Prep-HPLC21 | Prep-HPLC | Xselect CSH | A: Water (0.1% FA); | 254 nm/ |
| | | C18 OBD | B: ACN; | 220 nm |
| | | 30*75 mm | Flow rate: 60 mL/min | |
| | | 3.5 μm | | |
| Prep-HPLC22 | Prep-HPLC | YMC-Actus | A: Water (0.1% FA); | 254 nm/ |
| | | Triart C18, | B: ACN; | 220 nm |
| | | 20*250 mm, | Flow rate: 25 mL/min | |
| | | 5 μm | | |
| Prep-HPLC23 | Prep-HPLC | XBridge Prep | A: Water(10 mM | 254 nm/ |
| | | OBD C18 | NH₄HCO₃ + 0.05% | 220 nm |
| | | Column, | NH₃H₂O); | |
| | | 30*75 mm, | B: ACN; | |
| | | 7.5 μm | Flow rate: 60 mL/min | |
| Prep-HPLC24 | Prep-HPLC | XBridge Prep | A: Water (0.1% FA); | 254 nm/ |
| | | OBD C18, | B: ACN; | 220 nm |
| | | 30*150 mm, | Flow rate: 60 mL/min | |
| | | 5 μm | | |
| Prep-HPLC25 | Prep-HPLC | Xselect CSH | A: Water (0.1% FA); | 254 nm/ |
| | | C18 OBD | B: ACN; | 220 nm |
| | | 19*250 mm | Flow rate: 60 mL/min | |
| | | 5 μm | | |
| HP-FLASH1 | HP-Flash | YM C18 | A: Water (10 mmol | 254 nm/ |
| | | 50*150 mm, | NH₄HCO₃); | 220 nm |
| | | 10 μm | B: ACN; | |
| | | | Flow rate: 80 mL/min | |
| HP-FLASH2 | HP-Flash | YM 50*150 | A: Water (10 mmol | 254 nm/ |
| | | mm, 10 μm | NH₄HCO₃) | 220 nm |
| | | | B: ACN | |
| | | | Flow rate: 80 mL/min | |
| CHIRALPAK3 | Chiral HPLC | CHIRALPAK3 | Solvent indicated; | 254 nm/ |
| | | | Flow rate: 1 mL/min | 220 nm |
| CHIRALPAKIE | Chiral HPLC | CHIRALPAKIE | Solvent indicated; | 254 nm/ |
| | | 2*25 mm, 5 | Flow rate: 20 mL/min | 220 nm |
| | | μm | | |
| CHIRALPAKIF | Chiral HPLC | CHIRALPAKIF | Solvent indicated; | 254 nm/ |
| | | 2*25 mm, 5 | Flow rate: 20 mL/min | 220 nm |
| | | μm | | |
| CHIRALPAKIH | Chiral HPLC | CHIRALPAKIH-3 | Solvent indicated; | 254 nm/ |
| | | 4.6*50 mm, 3 | Flow rate: 4 mL/min | 220 nm |
| | | μm | | |
| CHIRALPAKIM | Chiral HPLC | CHIRALPAKIM | Solvent indicated; | 254 nm/ |
| | | 2*25 cm, 5 | Flow rate: 20 mL/min | 220 nm |
| | | μm | | |
| CHIRALART | Chiral HPLC | CHIRALART | Solvent indicated; | 254 nm/ |
| | | CelluloseZ | Flow rate: 1 or 20 mL/min | 220 nm |
| CHIRALART2 | Chiral HPLC | CHIRALART | Solvent indicated; | 254 nm/ |
| | | Cellulose-SZ2 | Flow rate: 20 mL/min | 220 nm |
| | | 2*25 cm, 5 | | |
| | | μm | | |
| CHIRALPAKIK | Chiral HPLC | CHIRALPAKIK | Solvent indicated; | 254 nm/ |
| | | 2*25 cm, 5 | Flow rate: 18 or 40 mL/min | 220 nm |
| | | μm | | |
| CHIRALPAKIK3 | Chiral HPLC | CHIRALPAKIK | Solvent indicated; | 254 nm/ |
| | | 3*25 cm, 5 | Flow rate: 30 mL/min | 220 nm |
| | | μm | | |
| CHIRALLUX | Chiral HPLC | Lux 5 μm | Solvent indicated; | 254 nm/ |
| | | Cellulose-2- | Flow rate: 22 mL/min | 220 nm |
| | | 2.12*25 cm, 5 | | |
| | | μm | | |
| CHIRALPAKIG | Chiral HPLC | CHIRALPAKIG | Solvent indicated; | 254 nm/ |
| | | 2*25 cm, | rate: 25 mL/min | 220 nm |
| | | 5 μm | | |
| CHIRALPAKIC | Chiral HPLC | CHIRALPAKIC3 | Solvent indicated; | 254 nm/ |
| | | | rate: 1 mL/min | 220 nm |
| NPFC1 | Normal-phase | — | Solvent indicated | 254 nm/ |
| | flash | | | 220 nm |
| | chromatography | | | |
| Prep-SFC1 | Prep-SFC | DAICEL | Solvent indicated; | 254 nm |
| | | DCpak P4VP | Rate: 60 mL/min | |
| | | 3*25 cm, | Column Temperature: 35° C. | |
| | | 5 μm | Back pressure: 100 bar | |

### Intermediates

Known intermediates

| Structure | CAS no. |
|---|---|
| 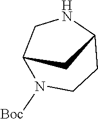 [SMILES: CC(C)(C)OC(=O)N1CC[C@@H]2C[C@H]1CN2] | 1932168-90-5 |
| 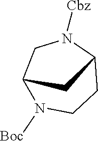 [SMILES: CC(C)(C)OC(=O)N1CC[C@@H]2C[C@H]1CN2C(=O)OCc1ccccc1] | 2178069-32-2 |
| 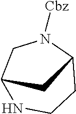 [SMILES: O=C(OCc1ccccc1)N1C[C@@H]2C[C@H]1CCN2] | 2178069-31-1 |
| 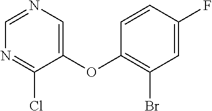 [SMILES: Fc1ccc(Oc2cncnc2Cl)c(Br)c1] | 2169919-47-3 |
| 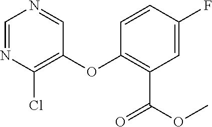 [SMILES: COC(=O)c1cc(F)ccc1Oc1cncnc1Cl] | 2169920-28-7 |
| 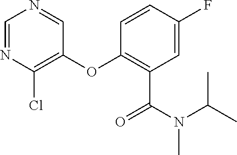 [SMILES: CC(C)N(C)C(=O)c1cc(F)ccc1Oc1cncnc1Cl] | 2169920-13-0 |
| 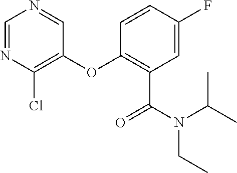 [SMILES: CCN(C(=O)c1cc(F)ccc1Oc1cncnc1Cl)C(C)C] | 2169923-04-8 |
| 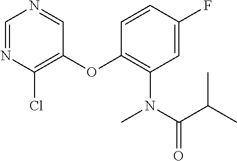 [SMILES: CC(C)C(=O)N(C)c1cc(F)ccc1Oc1cncnc1Cl] | 2169920-13-0 |
| 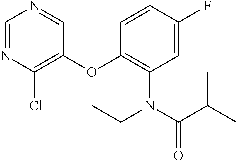 [SMILES: CCN(C(=O)C(C)C)c1cc(F)ccc1Oc1cncnc1Cl] | 2169923-04-8 |
| 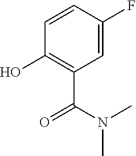 | 898541-62-3 |
| 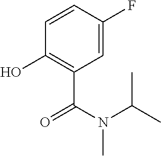 | 1553111-45-7 |
| 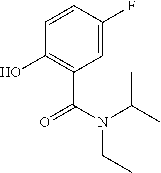 [SMILES: CCN(C(=O)c1cc(F)ccc1O)C(C)C] | 1549490-65-4 |
| 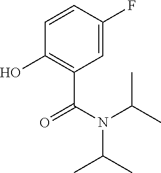 [SMILES: CC(C)N(C(=O)c1cc(F)ccc1O)C(C)C] | 1394933-63-1 |
| 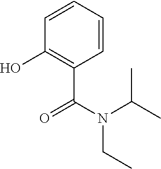 | 1094798-44-3 |
| | 2952717-09-6 |
| 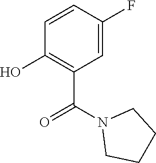 [SMILES: O=C(c1cc(F)ccc1O)N1CCCC1] | 1394954-57-4 |
| 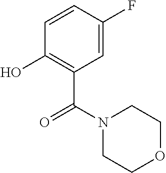 [SMILES: O=C(c1cc(F)ccc1O)N1CCOCC1] | 1394917-49-7 |
| 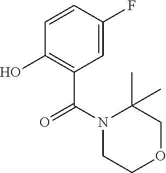 [SMILES: CC1(C)COCCN1C(=O)c1cc(F)ccc1O] | 1545340-23-5 |
| 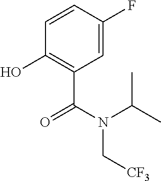 [SMILES: CC(C)N(CC(F)(F)F)C(=O)c1cc(F)ccc1O] | 1546029-38-2 |
| 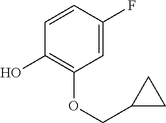 [SMILES: Oc1ccc(F)cc1OCC1CC1] | 1243456-68-9 |
| 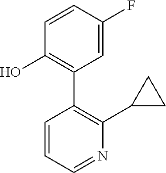 [SMILES: Oc1ccc(F)cc1-c1cccnc1C1CC1] | 2866314-76-1 |
| 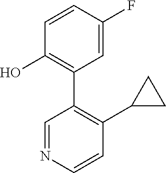 [SMILES: Oc1ccc(F)cc1-c1cnccc1C1CC1] | 2866315-14-0 |
| 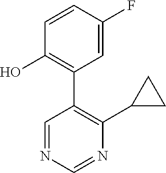 [SMILES: Oc1ccc(F)cc1-c1cncnc1C1CC1] | 2654082-97-8 |
| 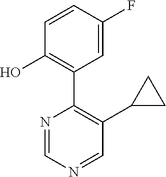 [SMILES: Oc1ccc(F)cc1-c1ncncc1C1CC1] | 2866315-22-0 |
| 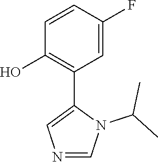 [SMILES: CC(C)n1cncc1-c1cc(F)ccc1O] | 2169922-02-3 |
| 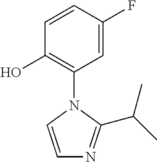 [SMILES: CC(C)c1nccn1-c1cc(F)ccc1O] | 2169922-02-3 |
| 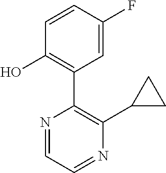 [SMILES: Oc1ccc(F)cc1-c1nccnc1C1CC1] | 2866315-20-8 |
| 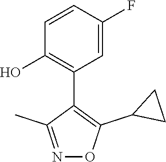 [SMILES: Cc1noc(C2CC2)c1-c1cc(F)ccc1O] | 2866316-32-5 |
| 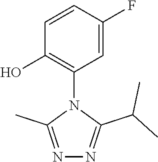 [SMILES: Cc1nnc(C(C)C)n1-c1cc(F)ccc1O] | 2866316-27-8 |
| 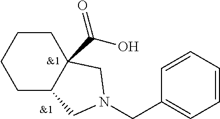 [SMILES: O=C(O)[C@]12CCCC[C@H]1CN(Cc1ccccc1)C2] | 2307772-61-6 |
| 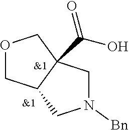 [SMILES: O=C(O)[C@]12COC[C@H]1CN(Cc1ccccc1)C2] | 2380998-82-1 |
| 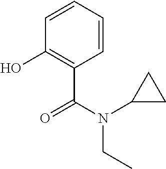 | 1550037-63-2 |
| 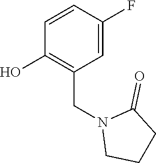 [SMILES: O=C1CCCN1Cc1cc(F)ccc1O] | 3103505-05-8 |
| 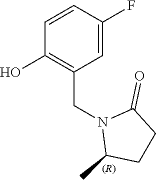 | 3103505-07-0 |

### N-ethyl-N-(5-fluoro-2-hydroxyphenyl)cyclopropanecarboxamide

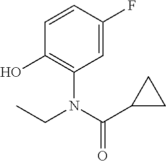 [SMILES: CCN(C(=O)C1CC1)c1cc(F)ccc1O]

### Step 1: N-(5-fluoro-2-methoxyphenyl)cyclopropanecarboxamide

To a stirred solution of 5-fluoro-2-methoxyaniline (3.0 g, 21 mmol, 1.0 equiv) and NEt₃ (4.3 g, 42.5 mmol, 2.0 equiv) in DCM (30 mL) were added cyclopropanecarbonyl chloride (2.2 g, 21 mmol, 1.0 equiv) dropwise at 0° C. under nitrogen. The resulting mixture was stirred at room temperature for 2 h under nitrogen. The resulting mixture was diluted with water (30 mL), and extracted with DCM (3×80 mL). The combined organic layers were washed with brine (20 mL), dried over Na₂SO₄, and concentrated under reduced pressure. The residue was purified by silica gel column chromatography, eluting with PE/EtOAC (1:1) to afford N-(5-fluoro-2-methoxyphenyl)cyclopropanecarboxamide (4.0 g, 90%) as a light yellow oil. m/z: ES⁺ [M+H]⁺=201.10.

### Step 2: N-ethyl-N-(5-fluoro-2-methoxyphenyl)cyclopropanecarboxamide

To a stirred solution of N-(5-fluoro-2-methoxyphenyl)cyclopropanecarboxamide (3.0 g, 14 mmol, 1.0 equiv) in THF (30 mL) were added NaH (1.0 g, 43 mmol, 3.0 equiv) dropwise at 0° C. under nitrogen. The resulting mixture was stirred at room temperature for 30 min, and ethyl iodide (2.7 g, 17 mmol, 1.2 equiv) was added at room temperature. The resulting mixture was stirred at room temperature for additional 5 h under nitrogen, and then diluted with water (100 mL) and extracted with EtOAc (3×100 mL). The combined organic layers were washed with brine (100 mL), dried over Na₂SO₄, and concentrated under reduced pressure. The residue was purified by silica gel column chromatography, eluting with PE/EtOAC (1:1) to afford N-ethyl-N-(5-fluoro-2-methoxyphenyl)cyclopropanecarboxamide (3.3 g, 97%) as a light yellow oil. m/z: ES⁺ [M+H]⁺=238.10.

### Step 3: N-ethyl-N-(5-fluoro-2-hydroxyphenyl)cyclopropanecarboxamide

To a stirred solution of N-ethyl-N-(5-fluoro-2-methoxyphenyl)cyclopropanecarboxamide (1.4 g, 6.1 mmol, 1.0 equiv) in DCM (14 mL) was added BBr₃ (4.6 g, 18 mmol, 3.0 equiv) dropwise at 0° C. under nitrogen. The resulting mixture was stirred at room temperature for 5 h under nitrogen and then quenched by the addition of MeOH (2 mL) at 0° C. The resulting mixture was diluted with water and extracted with DCM (2×10 mL). The combined organic layers were washed with brine (10 mL), dried over Na₂SO₄, and concentrated under reduced pressure. The residue was purified using method RPFC1 (40% to 50% gradient in 10 min) to afford N-ethyl-N-(5-fluoro-2-hydroxyphenyl)cyclopropanecarboxamide (600 mg, 44%) as a light yellow oil. m/z: ES⁺ [M+H]⁺=224.10.

### N-(5-fluoro-2-hydroxyphenyl)-N-isopropylcyclopropanecarboxamide

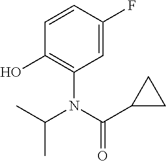 [SMILES: CC(C)N(C(=O)C1CC1)c1cc(F)ccc1O]

### Step 1: N-(5-fluoro-2-methoxyphenyl)-N-isopropylcyclopropanecarboxamide

To a stirred solution of 5-fluoro-N-isopropyl-2-methoxyaniline (1.5 g, 8.2 mmol, 1.0 equiv) and NEt₃ (2.5 g, 25 mmol, 3.0 equiv) in DCM (20 mL) was added cyclopropanecarbonyl chloride (0.94 g, 9.0 mmol, 1.1 equiv) dropwise at 0° C. under nitrogen. The resulting mixture was stirred at room temperature for 1 h, diluted with water (100 mL), and was extracted with DCM (3×50 mL). The combined organic layers were washed with brine (30 mL), dried over Na₂SO₄, and concentrated under reduced pressure. The residue was purified using method RPFC4 (45% to 50% gradient in 10 min) to afford N-(5-fluoro-2-methoxyphenyl)-N-isopropyl cyclopropanecarboxamide (1.5 g, 73%) as a light yellow oil. m/z: ES⁺ [M+H]⁺=252.10.

### Step 2: N-(5-fluoro-2-hydroxyphenyl)-N-isopropylcyclopropanecarboxamide

To a stirred solution of N-(5-fluoro-2-methoxyphenyl)-N-isopropylcyclopropanecarboxamide (1.3 g, 5.2 mmol, 1.0 equiv) in DCM (13 mL) were added BBr₃ (3.9 g, 16 mmol, 3.0 equiv) dropwise at 0° C. under nitrogen. The resulting mixture was stirred at room temperature overnight and quenched with sat. NaHCO₃ at 0° C., and extracted with DCM (3×50 mL). The combined organic layers were washed with brine (20 mL), dried over Na₂SO₄, and concentrated under reduced pressure. The residue was purified using method RPFC4 (70% to 80% gradient in 10 min) to afford N-(5-fluoro-2-hydroxyphenyl)-N-isopropylcyclopropanecarboxamide (200 mg, 16%) as a light yellow oil. m/z: ES⁺ [M+H]⁺=238.15.

### N-(5-fluoro-2-hydroxyphenyl)-N-isopropylisobutyramide

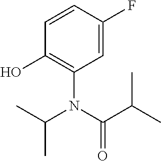 [SMILES: CC(C)C(=O)N(c1cc(F)ccc1O)C(C)C]

### Step 1: N-(5-fluoro-2-methoxyphenyl)-N-isopropylisobutyramide

A solution of 5-fluoro-N-isopropyl-2-methoxyaniline (1.1 g, 5.8 mmol, 1.0 equiv) and isobutyryl chloride (0.74 g, 6.9 mmol, 1.2 equiv) in pyridine (10 mL) was stirred at room temperature for 2 h. The resulting mixture was diluted with water (50 mL) and extracted with DCM (3×50 mL). The combined organic layers were dried over Na₂SO₄ and filtered. The filtrate was concentrated under vacuum and the residue purified by RPFC1 (0% to 100% gradient in 30 min) to afford N-(5-fluoro-2-methoxyphenyl)-N-isopropylisobutyramide (1.2 g, 82%) as a colourless solid. m/z: ES⁺ [M+H]⁺=254.10.

### Step 2: N-(5-fluoro-2-hydroxyphenyl)-N-isopropylisobutyramide

A solution of N-(5-fluoro-2-methoxyphenyl)-N-isopropylisobutyramide (500 mg, 2.0 mmol, 1.0 equiv) and BBr₃ (2 mL, 0.47 mmol, 4.0 equiv) in DCM (0.5 mL) was stirred at room temperature for 16 h. The resulting mixture was diluted with water (10 mL) and extracted with DCM (3×5 mL). The combined organic layers were dried over Na₂SO₄ and filtered. The filtrate was concentrated under reduced pressure and used in the next step directly without further purification. m/z: ES⁺ [M+H]⁺=240.00.

### N-cyclopropyl-N-(5-fluoro-2-hydroxyphenyl)cyclopropanecarboxamide

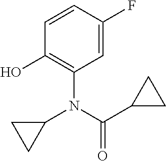 [SMILES: O=C(C1CC1)N(c1cc(F)ccc1O)C1CC1]

### Step 1: 2-(benzyloxy)-N-cyclopropyl-5-fluoroaniline

To a stirred mixture of 1-(benzyloxy)-2-bromo-4-fluorobenzene (2.5 g, 8.9 mmol, 1.0 equiv) and cyclopropanamine (0.76 g, 13 mmol, 1.5 equiv) in toluene (40 mL) were added Pd(dppf)Cl₂CH₂Cl₂ (0.73 g, 0.89 mmol, 0.1 equiv), BINAP (1.1 g, 1.8 mmol, 0.2 equiv) and tBuONa (1.7 g, 18 mmol, 2.0 equiv) in portions at room temperature under nitrogen. The resulting mixture was stirred at 80° C. for 16 h under nitrogen, and then diluted with water and extracted with EtOAc (3×50 mL). The combined organic layers were washed with brine (3×20 mL), dried over Na₂SO₄, and concentrated under reduced pressure. The residue was purified using method RPFC1 (0% to 100% gradient in 30 min) to afford 2-(benzyloxy)-N-cyclopropyl-5-fluoroaniline (1.4 g, 61%) as a yellow oil. m/z: ES⁺ [M+H]⁺=258.15.

### Step 2: N-(2-(benzyloxy)-5-fluorophenyl)-N-cyclopropylcyclopropane carboxamide

To a stirred mixture of 2-(benzyloxy)-N-cyclopropyl-5-fluoroaniline (600 mg, 2.3 mmol, 1.0 equiv) and cyclopropanecarbonyl chloride (487 mg, 4.7 mmol, 2.0 equiv) in DCM (10 mL) was added NEt₃ (708 mg, 7.0 mmol, 3.0 equiv) at room temperature. The resulting mixture was stirred at room temperature for 1 h, and then quenched with water at 0° C. and extracted with EtOAc (3×20 mL). The combined organic layers were washed with brine (3×10 mL), dried over Na₂SO₄, and concentrated under reduced pressure. The residue was purified using method RPFC1 (0% to 100% gradient in 30 min) to afford N-(2-(benzyloxy)-5-fluorophenyl)-N-cyclopropylcyclopropanecarboxamide (480 mg, 63%) as a yellow oil. m/z: ES⁺ [M+H]⁺=326.20.

### Step 3: N-cyclopropyl-N-(5-fluoro-2-hydroxyphenyl)cyclopropane carboxamide

A mixture of N-(2-(benzyloxy)-5-fluorophenyl)-N-cyclopropylcyclopropanecarboxamide (460 mg, 1.4 mmol, 1.0 equiv) and Pd/C (230 mg, 10% wt, wet) in EtOAc (10 mL) was stirred at room temperature for 2 h under hydrogen. The resulting mixture was then filtered, and the filter cake was washed with MeOH (3×10 mL). The filtrate was concentrated under reduced pressure to afford N-cyclopropyl-N-(5-fluoro-2-hydroxyphenyl)cyclopropanecarboxamide (320 mg, 96%) as a yellow solid. m/z: ES⁺ [M+H]⁺=236.20.

### 2-(3-(ethyl(isopropyl)amino)oxetan-3-yl)-4-fluorophenol

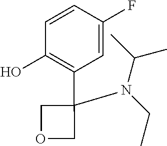 [SMILES: CCN(C(C)C)C1(c2cc(F)ccc2O)COC1]

### Step 1: N-(3-(2-(benzyloxy)-5-fluorophenyl)oxetan-3-yl)-2-methylpropane-2-sulfinamide

A solution of 1-(benzyloxy)-2-bromo-4-fluorobenzene (2.0 g, 7.1 mmol, 1.0 equiv) and nBuL₁ (0.91 g, 14 mmol, 2.0 equiv) in THF (20 mL) was stirred at −78° C. for 2 h under nitrogen. 2-methyl-N-(oxetan-3-ylidene)propane-2-sulfinamide (2.5 g, 14 mmol, 2.0 equiv) was then added and the reaction allowed to warm to room temperature and stirred for 16 h under nitrogen. The reaction was quenched with sat. NH₄Cl, and extracted with DCM (2×100 mL). The combined organic layers were washed with brine (2×100 mL), dried over Na₂SO₄, and concentrated under reduced pressure. The residue was purified by RPFC1 (0% to 100% gradient in 20 min) to afford N-(3-(2-(benzyloxy)-5-fluorophenyl)oxetan-3-yl)-2-methylpropane-2-sulfinamide (800 mg, 30%) as a colourless solid. m/z: ES⁺ [M+H]⁺=378.15.

### Step 2: 3-(2-(benzyloxy)-5-fluorophenyl)oxetan-3-amine

A solution of N-(3-(2-(benzyloxy)-5-fluorophenyl)oxetan-3-yl)-2-methylpropane-2-sulfinamide (700 mg, 1.9 mmol, 1.0 equiv) and TFA (2 mL) in DCM (7 mL) was stirred at room temperature for 2 h. The resulting mixture was concentrated under reduced pressure and the residue purified by RPFC3 (0% to 100% gradient in 15 min) to afford 3-(2-(benzyloxy)-5-fluorophenyl)oxetan-3-amine (410 mg, 81%) as a yellow oil. m/z: ES⁺ [M+H]⁺=274.12.

### Step 3: 3-(2-(benzyloxy)-5-fluorophenyl)-N-isopropyloxetan-3-amine

A solution of 3-(2-(benzyloxy)-5-fluorophenyl)oxetan-3-amine (400 mg, 1.5 mmol, 1.0 equiv), acetone (94 mg, 1.6 mmol, 1.1 equiv), Ti(OEt)₄ (3.0 g, 13 mmol, 9.0 equiv) and NaBH₃CN (276 mg, 4.4 mmol, 3.0 equiv) in MeOH (5 mL) was stirred at room temperature for 16 h under nitrogen. The reaction was quenched with sat. NH₄Cl, and extracted with DCM (2×20 mL). The combined organic layers were washed with brine (2×40 mL), dried over Na₂SO₄, and concentrated under reduced pressure. The residue was purified by RPFC1 (0% to 100% gradient in 25 min) to afford 3-(2-(benzyloxy)-5-fluorophenyl)-N-isopropyloxetan-3-amine (220 mg, 48%) as a yellow oil. m/z: ES⁺ [M+H]⁺=316.16.

### Step 4: 3-(2-(benzyloxy)-5-fluorophenyl)-N-ethyl-N-isopropyloxetan-3-amine

A solution of 3-(2-(benzyloxy)-5-fluorophenyl)-N-isopropyloxetan-3-amine (140 mg, 0.44 mmol, 1.0 equiv), acetaldehyde (196 mg, 4.4 mmol, 10 equiv), Na₂SO₄ (19 mg, 0.13 mmol, 0.3 equiv) and STAB (282 mg, 1.3 mmol, 3.0 equiv) in DCM (1.5 mL) was stirred at room temperature for 2 h. The reaction was quenched with sat. NH₄Cl, and extracted with DCM (2×20 mL). The combined organic layers were washed with brine (2×40 mL), dried over Na₂SO₄, and concentrated under reduced pressure. The residue was purified by RPFC4 (0% to 100% gradient in 25 min) to afford 3-(2-(benzyloxy)-5-fluorophenyl)-N-ethyl-N-isopropyloxetan-3-amine (110 mg, 72%) as a yellow oil. m/z: ES⁺ [M+H]⁺=344.19.

### Step 5: 2-(3-(ethyl(isopropyl)amino)oxetan-3-yl)-4-fluorophenol

A solution of 3-(2-(benzyloxy)-5-fluorophenyl)-N-ethyl-N-isopropyloxetan-3-amine (100 mg, 0.29 mmol, 1.0 equiv) and Pd/C (50 mg, 10% wt, wet) in MeOH (1.5 mL) was stirred at room temperature for 2 h under hydrogen. The resulting mixture was filtered, the filter cake washed with MeOH (3×3 mL) and the filtrate concentrated under reduced pressure. The crude product was used in the next step directly without further purification. m/z: ES⁺ [M+H]⁺=254.15.

### 2-(4-cyclopropyl-2-methylpyridin-3-yl)-4-fluorophenol

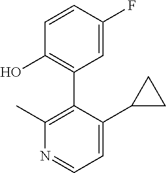 [SMILES: Cc1nccc(C2CC2)c1-c1cc(F)ccc1O]

### Step 1: 3-(2-(benzyloxy)-5-fluorophenyl)-4-chloro-2-methylpyridine

A solution of 3-bromo-4-chloro-2-methylpyridine (150 mg, 0.73 mmol, 1.0 equiv), (2-(benzyloxy)-5-fluorophenyl)boronic acid (268 mg, 1.1 mmol, 1.5 equiv), Pd(PPh₃)₄ (84 mg, 0.07 mmol, 0.1 equiv) and K₃PO₄ (463 mg, 2.2 mmol, 3.0 equiv) in dioxane (0.7 mL) and water (0.1 mL) was stirred at 80° C. for 16 h under nitrogen. The resulting mixture was diluted with water (10 mL), extracted with DCM (3×10 mL), and dried over Na₂SO₄. After filtration, the filtrate was concentrated under reduced pressure, and purified using method RPFC1 (0% to 100% gradient in 20 min). This resulted in 3-(2-(benzyloxy)-5-fluorophenyl)-4-chloro-2-methylpyridine (200 mg, 84%) as a yellow oil. m/z: ES⁺ [M+H]⁺=328.08.

### Step 2: 3-(2-(benzyloxy)-5-fluorophenyl)-4-cyclopropyl-2-methylpyridine

A solution of 3-(2-(benzyloxy)-5-fluorophenyl)-4-chloro-2-methylpyridine (100 mg, 0.31 mmol, 1.0 equiv), cyclopropylboronic acid (52 mg, 0.61 mmol, 2.0 equiv), Pd(OAc)₂ (7.0 mg, 0.03 mmol, 0.1 equiv), PCy₃ (17 mg, 0.06 mmol, 0.2 equiv) and K₃PO₄ (194 mg, 0.92 mmol, 3.0 equiv) in toluene (1 mL) was stirred at 80° C. for 16 h under nitrogen. The resulting mixture was diluted with water (10 mL), extracted with DCM (3×5 mL), dried over Na₂SO₄, and concentrated under reduced pressure, and purified using method RPFC1 (0% to 100% gradient in 20 min). This resulted in 3-(2-(benzyloxy)-5-fluorophenyl)-4-chloro-2-methylpyridine (100 mg, 98%) as a light yellow oil. m/z: ES⁺ [M+H]⁺=334.15.

### Step 3: 2-(4-cyclopropyl-2-methylpyridin-3-yl)-4-fluorophenol

A solution of 3-(2-(benzyloxy)-5-fluorophenyl)-4-cyclopropyl-2-methylpyridine (130 mg, 0.39 mmol, 1.0 equiv) and Pd/C (25 mg, 10% wt, wet) in EtOAc (3 mL) was stirred at room temperature for 16 h under hydrogen. The resulting mixture was filtered, the filter cake was washed with MeOH (3×5 mL), and concentrated under reduced pressure. This resulted in 2-(4-cyclopropyl-2-methylpyridin-3-yl)-4-fluorophenol (80 mg, 84%) as a colourless solid. m/z: ES⁺ [M+H]⁺=244.11.

### 2-(2-cyclopropyl-4-methylpyridin-3-yl)-4-fluorophenol

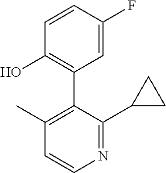 [SMILES: Cc1ccnc(C2CC2)c1-c1cc(F)ccc1O]

### Step 1: 3-bromo-2-cyclopropyl-4-methylpyridine

A solution of 2,3-dibromo-4-methylpyridine (3.4 g, 14 mmol, 1.0 equiv), cyclopropylboronic acid (1.7 g, 20 mmol, 1.5 equiv), K₃PO₄ (5.7 g, 27 mmol, 2.0 equiv) and Pd(PPh₃)₄ (3.1 g, 2.7 mmol, 0.2 equiv) in water (3.5 mL) and 1,4-dioxane (35 mL) was stirred at room temperature under nitrogen. The resulting mixture was stirred at 80° C. for 16 h under nitrogen, and then diluted with water (10 mL) and extracted with DCM. The combined organic layers were washed with brine, dried over Na₂SO₄, and concentrated under reduced pressure. The residue was purified using method RPFC1 (10% to 100% gradient in 25 min) to afford 3-bromo-2-cyclopropyl-4-methylpyridine (250 mg, 9%) as a brown solid. m/z: ES⁺ [M+H]⁺=212.05.

### Step 2: 2-(2-cyclopropyl-4-methylpyridin-3-yl)-4-fluorophenol

A solution of 3-bromo-2-cyclopropyl-4-methylpyridine (200 mg, 0.94 mmol, 1.0 equiv), (5-fluoro-2-hydroxyphenyl)boronic acid (441 mg, 2.8 mmol, 3.0 equiv), K₃PO₄ (601 mg, 2.8 mmol, 3.0 equiv) and Pd(PPh₃)₄ (272 mg, 0.24 mmol, 0.25 equiv) in dioxane (3 mL) and water (0.5 mL) was stirred at room temperature under nitrogen. The resulting mixture was stirred at 80° C. for 16 h, and then diluted with water (10 mL) and extracted with DCM. The combined organic layers were washed with brine, dried over Na₂SO₄, and concentrated under reduced pressure. The residue was purified using method RPFC1 (10% to 100% gradient in 25 min) to afford 2-(2-cyclopropyl-4-methylpyridin-3-yl)-4-fluorophenol (310 mg, crude) as a grey-white viscous oil. m/z: ES⁺ [M+H]⁺=266.15.

### 4-fluoro-2-(2-methoxy-5-methyl-1H-imidazol-1-yl)phenol

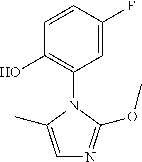

### Step 1: 1-(2-(benzyloxy)-5-fluorophenyl)-2-methoxy-1H-imidazole

A solution of 2-methoxy-1H-imidazole (1.5 g, 15 mmol, 1.0 equiv), (2-(benzyloxy)-5-fluorophenyl)boronic acid (3.8 g, 15 mmol, 1.0 equiv), Cu(OAc)₂ (0.56 g, 3.1 mmol, 0.2 equiv) and NEt₃ (4.6 g, 46 mmol, 3.0 equiv) in MeOH (20 mL) was stirred at room temperature. The resulting mixture was diluted with water (100 mL) and extracted with DCM (2×100 mL). The combined organic layers were washed with brine (2×150 mL), dried over Na₂SO₄, and concentrated under reduced pressure. The residue was purified using method RPFC1 (10% to 50% gradient in 10 min) to afford 1-(2-(benzyloxy)-5-fluorophenyl)-2-methoxy-1H-imidazole (600 mg, 13%) as a yellow oil. m/z: ES⁺ [M+H]⁺=299.05.

### Step 2: 1-(2-(benzyloxy)-5-fluorophenyl)-5-bromo-2-methoxy-1H-imidazole

A solution of 1-(2-(benzyloxy)-5-fluorophenyl)-2-methoxy-1H-imidazole (600 mg, 2.0 mmol, 1.0 equiv) and NBS (358 mg, 2.0 mmol, 1.0 equiv) in DMF (6 mL) was stirred at room temperature for 1 h. The residue was purified using method RPFC1 (10% to 50% gradient in 10 min) to afford 1-(2-(benzyloxy)-5-fluorophenyl)-5-bromo-2-methoxy-1H-imidazole (350 mg, 46%) as a light yellow oil. m/z: ES⁺ [M+H]+=378.90.

### Step 3: 1-(2-(benzyloxy)-5-fluorophenyl)-2-methoxy-5-methyl-1H-imidazole

A solution of 1-(2-(benzyloxy)-5-fluorophenyl)-5-bromo-2-methoxy-1H-imidazole (300 mg, 0.8 mmol, 1.0 equiv), trimethyl-1,3,5,2,4,6-trioxatriborinane (199 mg, 1.6 mmol, 2.0 equiv), RuPhos Pd G3 (66 mg, 0.08 mmol, 0.1 equiv), RuPhos (74 mg, 0.15 mmol, 0.2 equiv) and K₂CO₃ (219 mg, 1.6 mmol, 2.0 equiv) in 1,2-dimethoxyethane (1.0 mL) was stirred at 100° C. for 3 h under nitrogen. The resulting mixture was diluted with water (30 mL) and extracted with DCM (2×30 mL). The combined organic layers were washed with brine (2×50 mL), dried over Na₂SO₄, and concentrated under reduced pressure. The residue was purified using method RPFC1 (10% to 50% gradient in 10 min) to afford 1-(2-(benzyloxy)-5-fluorophenyl)-2-methoxy-5-methyl-1H-imidazole (150 mg, 60%) as a brown oil. m/z: ES⁺ [M+H]+=313.00.

### Step 4: 4-fluoro-2-(2-methoxy-5-methyl-1H-imidazol-1-yl)phenol

A solution of 1-(2-(benzyloxy)-5-fluorophenyl)-2-methoxy-5-methyl-1H-imidazole (130 mg, 0.41 mmol, 1.0 equiv) and Pd/C (80 mg, 10% wt, wet) in EtOAc (1 mL) was stirred at room temperature for 2 h under hydrogen (1 atm). The reaction mixture was filtered through a Celite pad and concentrated under reduced pressure to afford 4-fluoro-2-(2-methoxy-5-methyl-1H-imidazol-1-yl)phenol (80 mg, 86%) as a brown oil. m/z: ES⁺ [M+H]⁺=223.05.

### 4-fluoro-2-(2-isopropoxy-5-methyl-1H-imidazol-1-yl)phenol

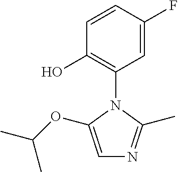 [SMILES: Cc1ncc(OC(C)C)n1-c1cc(F)ccc1O]

### Step 1: 1-(2-(benzyloxy)-5-fluorophenyl)-2-isopropoxy-1H-imidazole

A solution of 2-isopropoxy-1H-imidazole (2.5 g, 20 mmol, 1.0 equiv), 2-(benzyloxy)-5-fluorophenylboronic acid (5.9 g, 24 mmol, 1.2 equiv), NEt₃ (6.0 g, 60 mmol, 3.0 equiv) and Cu(OAc)₂ (0.72 g, 3.9 mmol, 0.2 equiv) in MeOH (40 mL) were stirred at room temperature for 16 h. The resulting mixture was concentrated under reduced pressure, diluted with water (150 mL) and extracted with EtOAc (2×250 mL). The combined organic layers were washed with brine (500 mL), dried over Na₂SO₄, and concentrated under reduced pressure. The residue was purified using method RPFC1 (40% to 80% gradient in 10 min) followed by Prep-HPLC1 (32% to 57% MeCN in 8 min; Rt=7.8 min) to afford 1-(2-(benzyloxy)-5-fluorophenyl)-2-isopropoxy-1H-imidazole (300 mg, 4.6%) as a yellow oil. m/z: ES⁺ [M+H]⁺=327.10.

### Step 2: 1-(2-(benzyloxy)-5-fluorophenyl)-5-bromo-2-isopropoxy-1H-imidazole

A solution of 1-(2-(benzyloxy)-5-fluorophenyl)-2-isopropoxy-1H-imidazole (300 mg, 0.91 mmol, 1.0 equiv) and NBS (130 mg, 0.73 mmol, 0.8 equiv) in DMF (5 mL) was stirred at room temperature for 2 h. The resulting mixture was diluted with water (30 mL) and extracted with EtOAc (2×80 mL). The combined organic layers were washed with brine (160 mL), dried over Na₂SO₄, and concentrated under reduced pressure. The residue was purified using method RPFC1 (40% to 80% gradient in 10 min) to give 1-(2-(benzyloxy)-5-fluorophenyl)-5-bromo-2-isopropoxy-1H-imidazole (300 mg, 81%) as a light yellow solid. m/z: ES⁺ [M+H]⁺=405.05.

### Step 3: 1-(2-(benzyloxy)-5-fluorophenyl)-2-isopropoxy-5-methyl-1H-imidazole

A solution of 1-(2-(benzyloxy)-5-fluorophenyl)-5-bromo-2-isopropoxy-1H-imidazole (290 mg, 0.71 mmol, 1.0 equiv), K₂CO₃ (270 mg, 2.1 mmol, 3.0 equiv), trimethyl-1,3,5,2,4,6-trioxatriborinane (180 mg, 1.4 mmol, 2.0 equiv), RuPhos (67 mg, 0.14 mmol, 0.2 equiv) and RuPhos Pd G₃ (90 mg, 0.1 mmol, 0.15 equiv) in 1,4-dioxane (5 mL) was stirred at 100° C. for 2 h under nitrogen. The resulting mixture was diluted with water (50 mL) and extracted with EtOAc (2×80 mL). The combined organic layers were washed with brine (150 mL), dried over anhydrous Na₂SO₄, and concentrated under reduced pressure. The residue was purified using method RPFC1 (20% to 80% gradient in 15 min) to give 1-(2-(benzyloxy)-5-fluorophenyl)-2-isopropoxy-5-methyl-1H-imidazole (200 mg, 82%) as a light yellow oil. m/z: ES⁺ [M+H]⁺=341.15.

### Step 4: 4-fluoro-2-(2-isopropoxy-5-methyl-1H-imidazol-1-yl)phenol

A solution of 1-(2-(benzyloxy)-5-fluorophenyl)-2-isopropoxy-5-methyl-1H-imidazole (190 mg, 0.6 mmol, 1.0 equiv) in EtOAc (6 mL) was added Pd/C (59 mg, 10% wt, wet) and the resulting mixture was stirred at room temperature for 2 h under hydrogen. The resulting mixture was filtered and the filter cake was washed with EtOAc (2×10 mL). The filtrate was concentrated under reduced pressure to give 4-fluoro-2-(2-isopropoxy-5-methyl imidazol-1-yl)phenol (120 mg, 86%) as a colourless solid. m/z: ES⁺ [M+H]⁺=251.15.

### 2-(2-cyclopropyl-5-isopropoxy-1H-imidazol-1-yl)-4-fluorophenol

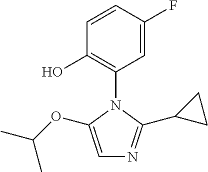 [SMILES: CC(C)Oc1cnc(C2CC2)n1-c1cc(F)ccc1O]

### Step 1: 1-(2-(benzyloxy)-5-fluorophenyl)-2-cyclopropyl-5-isopropoxy-1H-imidazole

A solution of 1-(2-(benzyloxy)-5-fluorophenyl)-5-bromo-2-cyclopropyl-1H-imidazole (100 mg, 0.25 mmol, 1.0 equiv) in iPrOH (2 mL) was treated with iPrONa (42 mg, 0.5 mmol, 2.0 equiv) at 25° C. for 5 min under nitrogen. CuI (10 mg, 0.05 mmol, 0.2 equiv) was then added portionwise at 25° C. and the resulting mixture stirred at 100° C. for 24 h under nitrogen. The resulting mixture was diluted with water (10 mL), extracted with EtOAc (2×25 mL) and the combined organic layers washed with brine (50 mL), dried over Na₂SO₄, and concentrated under reduced pressure. The residue was purified using method RPFC1 (20% to 80% gradient in 25 min) to give 1-(2-(benzyloxy)-5-fluorophenyl)-2-cyclopropyl-5-isopropoxy-1H-imidazole (25 mg, 26%) as a yellow solid. m/z: ES⁺ [M+H]⁺=367.10.

### Step 2: 2-(2-cyclopropyl-5-isopropoxy-1H-imidazol-1-yl)-4-fluorophenol

A solution of 1-(2-(benzyloxy)-5-fluorophenyl)-2-cyclopropyl-5-isopropoxy-1H-imidazole (50 mg, 0.13 mmol, 1.0 equiv) in EtOAc (2 mL) was stirred with Pd/C (20 mg, 10% wt, wet) at room temperature for 1 h under hydrogen. The resulting mixture was then filtered, the filter cake washed with EtOAc (2 mL) and the filtrate concentrated under reduced pressure to give 2-(2-cyclopropyl-5-isopropoxy-1H-imidazol-1-yl)-4-fluorophenol (30 mg, crude) as a yellow oil. m/z: ES⁺ [M+H]⁺=277.05.

### 4-fluoro-2-(5-isopropyl-2-methyl-1H-imidazol-1-yl)phenol

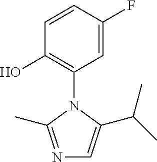 [SMILES: Cc1ncc(C(C)C)n1-c1cc(F)ccc1O]

### Step 1: 1-(2-(benzyloxy)-5-fluorophenyl)-2-methyl-5-(prop-1-en-2-yl)-1H-imidazole

A mixture of 1-(2-(benzyloxy)-5-fluorophenyl)-5-bromo-2-methyl-1H-imidazole (300 mg, 0.83 mmol, 1.0 equiv), 4,4,5,5-tetramethyl-2-(prop-1-en-2-yl)-1,3,2-dioxaborolane (419 mg, 2.5 mmol, 3.0 equiv), Pd(dppf)Cl₂ (61 mg, 0.083 mmol, 0.1 equiv) and K₂CO₃ (230 mg, 1.7 mmol, 2.0 equiv) in dioxane (4 mL) and water (0.5 mL) was stirred at 90° C. for 16 h under nitrogen. The resulting mixture was diluted with water (20 mL) and extracted with EtOAc (3×10 mL), dried over anhydrous Na₂SO₄, and concentrated under reduced pressure. The residue was purified using method RPFC1 (10% to 100% gradient in 20 min) to afford 1-(2-(benzyloxy)-5-fluorophenyl)-2-methyl-5-(prop-1-en-2-yl)-1H-imidazole (250 mg, 93%) as a brown oil. m/z: ES⁺ [M+H]⁺=323.15.

### Step 2: 4-fluoro-2-(5-isopropyl-2-methyl-1H-imidazol-1-yl)phenol

A mixture of 1-(2-(benzyloxy)-5-fluorophenyl)-2-methyl-5-(prop-1-en-2-yl)-1H-imidazole (200 mg, 0.62 mmol, 1.0 equiv) and Pd/C (50 mg, 10% wt, wet) in MeOH (5 mL) was stirred at room temperature for 2 h under hydrogen. The resulting mixture was filtered, the filter cake was washed with MeOH (3×4 mL). The filtrate was concentrated under reduced pressure to afford 4-fluoro-2-(5-isopropyl-2-methyl-1H-imidazol-1-yl)phenol (110 mg, 76%) as a yellow solid. m/z: ES⁺ [M+H]⁺=235.12.

### 2-(5-cyclopropyl-2-(trifluoromethyl)-1H-imidazol-1-yl)-4-fluorophenol

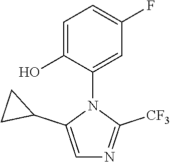 [SMILES: Oc1ccc(F)cc1-n1c(C2CC2)cnc1C(F)(F)F]

### Step 1: 1-(2-(benzyloxy)-5-fluorophenyl)-2-(trifluoromethyl)-1H-imidazole

To a stirred mixture of 2-(benzyloxy)-5-fluorophenylboronic acid (3.6 g, 15 mmol, 2.0 equiv) and 2-(trifluoromethyl)-1H-imidazole (1.0 g, 7.4 mmol, 1.0 equiv) in MeOH (20 mL) were added Cu(OAc)₂ (0.40 g, 2.2 mmol, 0.3 equiv) and NEt₃ (2.2 g, 22 mmol, 3.0 equiv) in portions at room temperature under air. The resulting mixture was stirred at room temperature for 16 h, and then filtered. The filter cake was washed with MeOH (3×30 mL), and the filtrate was concentrated under reduced pressure. The residue was purified by Prep-TLC (PE/EtOAC 3:1) to afford 1-(2-(benzyloxy)-5-fluorophenyl)-2-(trifluoromethyl)-1H-imidazole (260 mg, 11%) as a yellow solid. m/z: ES⁺ [M+H]⁺=337.05.

### Step 2: 1-(2-(benzyloxy)-5-fluorophenyl)-5-bromo-2-(trifluoromethyl)-1H-imidazole

A mixture of 1-(2-(benzyloxy)-5-fluorophenyl)-2-(trifluoromethyl)-1H-imidazole (700 mg, 2.1 mmol, 1.0 equiv) and NBS (370 mg, 2.1 mmol, 1.0 equiv) in MeCN (10 mL) was stirred at 80° C. for 2 h under nitrogen. The resulting mixture was then diluted with water (10 mL) and extracted with EtOAc (3×5 mL), dried over Na₂SO₄, and concentrated under reduced pressure. The residue was purified by Prep-TLC (PE/EtOAC 3:1) to afford 1-(2-(benzyloxy)-5-fluorophenyl)-5-bromo-2-(trifluoromethyl)-1H-imidazole (400 mg, 46%) as a yellow solid. m/z: ES⁺ [M+H]⁺=415.20.

### Step 3: 1-(2-(benzyloxy)-5-fluorophenyl)-5-cyclopropyl-2-(trifluoromethyl)-1H-imidazole

To a stirred mixture of 1-(2-(benzyloxy)-5-fluorophenyl)-5-bromo-2-(trifluoromethyl)-1H-imidazole (380 mg, 0.92 mmol, 1.0 equiv) and cyclopropylboronic acid (157 mg, 1.8 mmol, 2.0 equiv) in toluene (8 mL) were added Pd(OAc)₂ (21 mg, 0.09 mmol, 0.1 equiv) and tricyclohexylphosphane (51 mg, 0.18 mmol, 0.2 equiv) and K₃PO₄ (583 mg, 2.8 mmol, 3.0 equiv) in portions at room temperature under nitrogen. The resulting mixture was stirred at 80° C. for 2 h under nitrogen and then diluted with water (10 mL), and extracted with EtOAc (3×5 mL), dried over Na₂SO₄, and concentrated under reduced pressure. The residue was purified by Prep-TLC (PE/EtOAC 3:1) to afford 1-(2-(benzyloxy)-5-fluorophenyl)-5-cyclopropyl-2-(trifluoromethyl)-1H-imidazole (240 mg, 70%) as a colourless solid. m/z: ES⁺ [M+H]⁺=377.25.

### Step 4: 2-(5-cyclopropyl-2-(trifluoromethyl)-1H-imidazol-1-yl)-4-fluorophenol

To a stirred mixture of 1-(2-(benzyloxy)-5-fluorophenyl)-5-cyclopropyl-2-(trifluoromethyl)-1H-imidazole (220 mg, 0.59 mmol, 1.0 equiv) in MeOH (5 mL) was added Pd/C (56 mg, 10% wt, wet) at room temperature under nitrogen. The resulting mixture was stirred at room temperature for 2 h under hydrogen, and then was filtered. The filter cake was washed with MeOH (3×7 mL), and the filtrate was concentrated under reduced pressure to afford 2-(5-cyclopropyl-2-(trifluoromethyl)-1H-imidazol-1-yl)-4-fluorophenol (150 mg, 90%) as a yellow oil. m/z: ES⁺ [M+H]⁺=287.00.

### 5-cyclopropyl-6-(5-fluoro-2-hydroxyphenyl)-1-methylpyridin-2(1H)-one

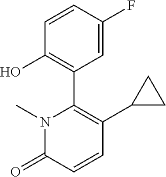 [SMILES: Cn1c(-c2cc(F)ccc2O)c(C2CC2)ccc1=O]

### Step 1: 6-chloro-5-cyclopropyl-1-methylpyridin-2(1H)-one

To a solution of 5-bromo-6-chloro-1-methylpyridin-2(1H)-one (1.0 g, 4.5 mmol, 1.0 equiv), cyclopropylboronic acid (0.31 g, 3.6 mmol, 0.8 equiv) and K₃PO₄ (0.19 g, 0.89 mmol, 0.2 equiv) in toluene (9 mL) and water (1 mL) were added Pd(OAc)₂ (0.15 g, 0.67 mmol, 0.15 equiv) and P(Cy)₃ (0.19 g, 0.67 mmol, 0.15 equiv) in portions at room temperature under nitrogen. The resulting mixture was stirred at 100° C. for 16 h. The resulting mixture was diluted with water (60 mL) and extracted with EtOAc (2×80 mL). The combined organic layers were washed with brine (120 mL), dried over Na₂SO₄, and concentrated under reduced pressure. The residue was purified using method RPFC4 (10% to 90% gradient in 30 min), resulting in 6-chloro-5-cyclopropyl-1-methylpyridin-2(1H)-one (550 mg, 66%) as a black oil. m/z: ES⁺ [M+H]⁺=183.90.

### Step 2: 6-(2-(benzyloxy)-5-fluorophenyl)-5-cyclopropyl-1-methylpyridin-2(1H)-one

RuPhos Pd G₃ (376 mg, 0.44 mmol, 0.15 equiv) and RuPhos (280 mg, 0.59 mmol, 0.2 equiv) were added to a stirred mixture of 6-chloro-5-cyclopropyl-1-methylpyridin-2(1H)-one (550 mg, 3.0 mmol, 1.0 equiv), 2-(benzyloxy)-5-fluorophenylboronic acid (737 mg, 2.9 mmol, 1.0 equiv) and K₂CO₃ (1.2 g, 9.0 mmol, 3.0 equiv) in toluene (9 mL) and water (1 mL) at room temperature under nitrogen. The resulting mixture was stirred at 100° C. for 4 h, diluted with water (60 mL) and extracted with EtOAc (2×70 mL). The combined organic layers were washed with brine (140 mL), dried over Na₂SO₄, and concentrated under reduced pressure. The residue was purified using method RPFC1 (10% to 90% gradient in 25 min). This resulted in 6-(2-(benzyloxy)-5-fluorophenyl)-5-cyclopropyl-1-methylpyridin-2(1H)-one (350 mg, 33%) as a black oil. m/z: ES⁺ [M+H]⁺=350.15

### Step 3: 5-cyclopropyl-6-(5-fluoro-2-hydroxyphenyl)-1-methylpyridin-2(1H)-one

A solution of tert-6-(2-(benzyloxy)-5-fluorophenyl)-5-cyclopropyl-1-methylpyridin-2(1H)-one (350 mg, 1.0 mmol, 1.0 equiv) and Pd/C (107 mg, 10% wt, wet) in EtOAc (10 mL) was stirred at room temperature for 1 h under hydrogen. The resulting mixture was filtered and the filter cake washed with EtOAc (20 mL). The filtrate was concentrated under reduced pressure to give 5-cyclopropyl-6-(5-fluoro-2-hydroxyphenyl)-1-methylpyridin-2(1H)-one (150 mg, 58%) as a colourless solid. m/z: ES⁺ [M+H]⁺=260.10.

### 6-(5-fluoro-2-hydroxyphenyl)-5-isopropyl-1-methylpyridin-2(1H)-one

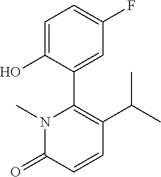 [SMILES: CC(C)c1ccc(=O)n(C)c1-c1cc(F)ccc1O]

### Step 1: 4-(5-fluoro-2-methoxybenzoyl)-5-methylhexanoic acid

A solution of 1-(5-fluoro-2-methoxyphenyl)-3-methylbutan-1-one (6.0 g, 28 mmol, 1.0 equiv), methyl acrylate (2.7 g, 31 mmol, 1.1 equiv) and tBuONa (3.0 g, 31 mmol, 1.1 equiv) in THF (50 mL) was stirred at room temperature for 16 h under nitrogen. The mixture was acidified to pH 5 with conc. HCl and extracted with DCM (2×50 mL). The combined organic layers were washed with brine (2×80 mL), dried over Na₂SO₄ and filtered. The filtrate was concentrated under reduced pressure to afford 4-(5-fluoro-2-methoxybenzoyl)-5-methylhexanoic acid (6.6 g, 82%) as a light yellow oil. m/z: ES⁺ [M+H]⁺=283.10.

### Step 2: 4-(5-fluoro-2-methoxybenzoyl)-N,5-dimethyihexanamide

4-(5-fluoro-2-methoxybenzoyl)-5-methylhexanoic acid (6.6 g, 23 mmol, 1.0 equiv) and MeNH₂ HCl (1.7 g, 26 mmol, 1.1 equiv) were coupled according to general procedure A1. Purification by RPFC4 (10% to 50% gradient in 30 min) to afford 4-(5-fluoro-2-methoxybenzoyl)-N,5-dimethylhexanamide (5.7 g, 83%) as a pink oil. m/z: ES⁺ [M+H]⁺=396.10.

### Step 3: 6-(5-fluoro-2-methoxyphenyl)-5-isopropyl-1-methyl-3,4-dihy dropyridin-2(11H)-one

A solution of 4-(5-fluoro-2-methoxybenzoyl)-N,5-dimethylhexanamide (5.7 g, 19.3 mmol, 1.0 equiv) and TsOH (3.3 g, 19 mmol, 1.0 equiv) in toluene (50 mL) was stirred at 100° C. for 6 h. The resulting mixture was diluted with water (100 mL) and extracted with DCM (2×100 mL). The combined organic layers were washed with brine (2×200 mL), dried over Na₂SO₄, and concentrated under reduced pressure to afford 6-(5-fluoro-2-methoxyphenyl)-5-isopropyl-1-methyl-3,4-dihydropyridin-2(1H)-one (4.7 g, 88%) as a brown oil. m/z: ES⁺ [M+H]⁺=283.15.

### Step 4: 6-(5-fluoro-2-methoxyphenyl)-5-isopropyl-1-methylpyridin-2(1H)-one

A solution of 6-(5-fluoro-2-methoxyphenyl)-5-isopropyl-1-methyl-3,4-dihydropyridin-2-one (7.0 g, 25 mmol, 1.0 equiv) and DDQ (12 g, 51 mmol, 2.0 equiv) in toluene (50 mL) was stirred at 110° C. for 8 h. The resulting mixture was diluted with water (200 mL) and extracted with DCM (2×200 mL). The combined organic layers were washed with brine (2×200 mL), dried over Na₂SO₄, and concentrated under reduced pressure. The residue was purified by RPFC4 (10% to 80% gradient in 25 min) to afford 6-(5-fluoro-2-methoxyphenyl)-5-isopropyl-1-methylpyridin-2(1H)-one (5.0 g, 72%) as a brown oil. m/z: ES⁺ [M+H]⁺=276.10.

### Step 5: 6-(5-fluoro-2-hydroxyphenyl)-5-isopropyl-1-methylpyridin-2(1H)-one

A solution of 6-(5-fluoro-2-methoxyphenyl)-5-isopropyl-1-methylpyridin-2(1H)-one (5.0 g, 18 mmol, 1.0 equiv) and BBr₃ (23 g, 91 mmol, 5.0 equiv) in DCM (50 mL) was stirred at room temperature for 16 h. The reaction was quenched by the addition of sat. NaHCO₃ (400 mL) at 0° C. and extracted with DCM (2×400 mL). The combined organic layers were washed with brine (2×800 mL), dried over Na₂SO₄, and concentrated under reduced pressure to afford 6-(5-fluoro-2-hydroxyphenyl)-5-isopropyl-1-methylpyridin-2(1H)-one (4.1 g, 86%) as a brown solid. m/z: ES⁺ [M+H]⁺=262.10.

### 6-(5-fluoro-2-hydroxyphenyl)-1-isopropyl-5-methylpyridin-2(1H)-one

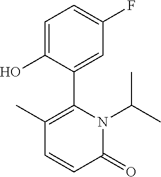 [SMILES: Cc1ccc(=O)n(C(C)C)c1-c1cc(F)ccc1O]

### Step 1: 5-(5-fluoro-2-methoxyphenyl)-4-methyl-5-oxopentanoic acid

tBuONa (23 g, 242 mmol, 1.1 equiv) was added to a stirred solution of 1-(5-fluoro-2-methoxyphenyl)propan-1-one (40 g, 220 mmol, 1.0 equiv), methyl acrylate (21 g, 242 mmol, 1.1 equiv) in THF (400 mL) at 0° C. under nitrogen. The mixture was allowed to stir at room temperature for 16 h under nitrogen, and then was acidified to pH=5 with conc. HCl. The mixture was extracted with EtOAc (2×200 mL), and the combined organic layers were washed with brine (2×200 mL), dried over Na₂SO₄ and concentrated under reduced pressure to afford 5-(5-fluoro-2-methoxyphenyl)-4-methyl-5-oxopentanoic acid. m/z: ES⁺ [M+H]⁺=255.30.

### Step 2: 5-(5-fluoro-2-methoxyphenyl)-N-isopropyl-4-methyl-5-oxopentanamide

5-(5-Fluoro-2-methoxyphenyl)-4-methyl-5-oxopentanoic acid (42 g, 165 mmol, 1.0 equiv) and propan-2-amine (11 g, 182 mmol, 1.1 equiv) were coupled according to general procedure A1. Purification using method RPFC4 (10% to 50% gradient in 10 min) yielded 5-(5-fluoro-2-methoxyphenyl)-N-isopropyl-4-methyl-5-oxopentanamide (17 g, 35%) as an orange oil. m/z: ES⁺ [M+H]⁺=296.15.

### Step 3: 6-(5-fluoro-2-methoxyphenyl)-1-isopropyl-5-methyl-3,4-dihydropyridin-2(1H)-one

A solution of 5-(5-fluoro-2-methoxyphenyl)-N-isopropyl-4-methyl-5-oxopentanamide (10 g, 39 mmol, 1.0 equiv) and TsOH (5.8 g, 34 mmol, 1 equiv) in toluene (15 mL) was stirred at 100° C. for 48 h. The resulting mixture was diluted with water (100 mL) and extracted with DCM (2×100 mL). The combined organic layers were washed with brine (2×200 mL), dried over Na₂SO₄, and concentrated under reduced pressure. The residue was purified using method RPFC1 (10% to 50% gradient in 10 min) to afford 6-(5-fluoro-2-methoxyphenyl)-1-isopropyl-5-methyl-3,4-dihydropyridin-2(1H)-one (1.5 g, 16%) as a brown oil. m/z: ES⁺ [M+H]⁺=278.15.

### Step 4: 6-(5-fluoro-2-methoxyphenyl)-1-isopropyl-5-methylpyridin-2(1H)-one

A solution of 6-(5-fluoro-2-methoxyphenyl)-1-isopropyl-5-methyl-3,4-dihydropyridin-2-one (1.5 g, 5.4 mmol, 1.0 equiv) and DDQ (2.5 g, 11 mmol, 2.0 equiv) in toluene (20 mL) was stirred at 100° C. for 8 h. The resulting mixture was diluted with water (200 mL) and extracted with DCM (2×200 mL). The combined organic layers were washed with brine (2×400 mL), dried over Na₂SO₄, and concentrated under reduced pressure. The residue purified by RPFC4 (10% to 50% gradient in 10 min) to afford 6-(5-fluoro-2-methoxyphenyl)-1-isopropyl-5-methylpyridin-2(1H)-one (950 mg, 64%) as a red oil. m/z: ES⁺ [M+H]⁺=276.20.

### Step 5: 6-(5-fluoro-2-hydroxyphenyl)-1-isopropyl-5-methylpyridin-2(1H)-one

A solution of 6-(5-fluoro-2-methoxyphenyl)-1-isopropyl-5-methylpyridin-2(1H)-one (950 mg, 3.5 mmol, 1.0 equiv) and BBr₃ (4.3 g, 17 mmol, 5.0 equiv) in DCM (10 mL) was stirred at room temperature for 16 h. The reaction was quenched by the addition of sat. NaHCO₃ (100 mL), and extracted with DCM (2×100 mL). The combined organic layers were washed with brine (2×200 mL), dried over Na₂SO₄, and concentrated under reduced pressure to afford 6-(5-fluoro-2-hydroxyphenyl)-1-isopropyl-5-methylpyridin-2(1H)-one (650 mg, 72%) as a brown solid. m/z: ES⁺ [M+H]⁺=262.30.

### 2-(2,5-dicyclopropyl-1H-pyrrol-1-yl)-4-fluorophenol

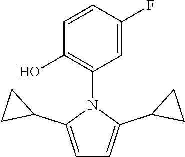 [SMILES: Oc1ccc(F)cc1-n1c(C2CC2)ccc1C1CC1]

### Step 1: 2,5-dicyclopropyl-1-(5-fluoro-2-methoxyphenyl)-1H-pyrrole

A solution of 1,4-dicyclopropylbutane-1,4-dione (200 mg, 1.2 mmol, 1.0 equiv) and 5-fluoro-2-methoxyaniline (170 mg, 1.2 mmol, 1.0 equiv) in HOAc (2 mL) was heated to 110° C. in a microwave for 5 h. The resulting mixture was diluted with water (10 mL), extracted with DCM (3×15 mL), and dried over Na₂SO₄. The resulting mixture was concentrated under vacuum, and purified using method RPFC2 (0% to 100% gradient in 30 min). This resulted in 2,5-dicyclopropyl-1-(5-fluoro-2-methoxyphenyl)-1H-pyrrole (200 mg, 61%) as a yellow oil. m/z: ES⁺ [M+H]⁺=272.15.

### Step 2: 2-(2,5-dicyclopropyl-1H-pyrrol-1-yl)-4-fluorophenol

The phenol deprotection of 2,5-dicyclopropyl-1-(5-fluoro-2-methoxyphenyl)-1H-pyrrole (150 mg, 0.55 mmol) was performed following step 2 of general procedure F1. The resulting mixture was extracted with DCM (3×10 mL), the combined organic layers were washed with brine (2×5 mL), and dried over Na₂SO₄. After filtration, the filtrate was concentrated under reduced pressure. The crude product was used in the next step directly without further purification. m/z: ES⁺ [M+H]⁺=256.00.

### 2-(3,5-dicyclopropyl-1,2,4-triazol-4-yl)-4-fluorophenol

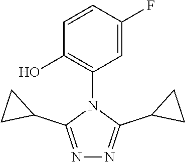 [SMILES: Oc1ccc(F)cc1-n1c(C2CC2)nnc1C1CC1]

### Step 1: N-(5-fluoro-2-methoxyphenyl)cyclopropanecarbothioamide

A solution of N-(5-fluoro-2-methoxyphenyl)cyclopropanecarboxamide (33 g, 158 mmol, 1.0 equiv) and Lawesson's reagent (32 g, 79 mmol, 0.5 equiv) in dioxane (330 mL) was stirred at 100° C. for 16 h under nitrogen. The resulting mixture was poured into water and extracted with DCM. The combined organic layers were washed with brine, dried over Na₂SO₄, and concentrated under reduced pressure. The residue was purified by silica gel column chromatography, eluted with PE/EtOAc (2:1) to afford N-(5-fluoro-2-methoxyphenyl)cyclopropanecarbothioamide (26 g, 73%) as a colourless solid. m/z: ES⁺ [M+H]⁺=225.95.

### Step 2: 3,5-dicyclopropyl-4-(5-fluoro-2-methoxyphenyl)-1,2,4-triazole

A solution of N-(5-fluoro-2-methoxyphenyl)cyclopropanecarbothioamide (26 g, 115 mmol, 1.0 equiv) and cyclopropanecarbohydrazide (58 g, 577 mmol, 5.0 equiv) in tBuOH (260 mL) was stirred at 130° C. for 48 h under nitrogen. The resulting mixture was concentrated under reduced pressure, and the residue was purified using method RPFC1 (10% to 100% gradient in 20 min), to afford 3,5-dicyclopropyl-4-(5-fluoro-2-methoxyphenyl)-1,2,4-triazole (20 g, 63%) as a colourless solid. m/z: ES⁺ [M+H]⁺=274.05.

### Step 3: 2-(3,5-dicyclopropyl-1,2,4-triazol-4-yl)-4-fluorophenol

To a stirred solution of 3,5-dicyclopropyl-4-(5-fluoro-2-methoxyphenyl)-1,2,4-triazole (20 g, 73 mmol, 1.0 equiv) in DCM (20 mL) was added BBr₃ (1 M in DCM, 293 mL, 293 mmol, 4.0 equiv) dropwise at 0° C. The resulting mixture was stirred at 25° C. for 16 h under nitrogen, and then poured into MeOH at 0° C. The resulting mixture was concentrated under reduced pressure, and purified by trituration with EtOAc. The precipitate was collected by filtration and washed with EtOAc to afford 2-(3,5-dicyclopropyl-1,2,4-triazol-4-yl)-4-fluorophenol (18 g, 95%) as a colourless solid. m/z: ES⁺ [M+H]⁺=260.05.

### 2-(3,5-diethyl-4H-1,2,4-triazol-4-yl)-4-fluorophenol

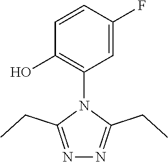 [SMILES: CCc1nnc(CC)n1-c1cc(F)ccc1O]

### Step 1: 4-(5-fluoro-2-methoxyphenyl)-4H-1,2,4-triazole

A solution of 5-fluoro-2-methoxy aniline (2.0 g, 23 mmol, 1.0 equiv), 5-fluoro-2-methoxyaniline (3.2 g, 23 mmol, 1.0 equiv) and TsOH (4.3 g, 25 mmol, 1.1 equiv) in toluene (20 mL) was stirred at 110° C. for 16 h under nitrogen. The resulting mixture was then concentrated under reduced pressure, and was purified using method RPFC1 (10% to 100% gradient in 20 min). This resulted in 4-(5-fluoro-2-methoxyphenyl)-4H-1,2,4-triazole (2.0 g, 46%) as an off-white solid. m/z: ES⁺ [M+H]⁺=194.06.

### Step 2: 3,5-dibromo-4-(5-fluoro-2-methoxyphenyl)-4H-1,2,4-triazole

A solution of 4-(5-fluoro-2-methoxyphenyl)-4H-1,2,4-triazole (2.0 g, 10 mmol, 1.0 equiv) and NBS (5.5 g, 31 mmol, 3.0 equiv) in DMF (30 mL) was stirred at 80° C. for 16 h under nitrogen. The resulting mixture was diluted with water and extracted with EtOAc. The combined organic layers were washed with brine, dried over Na₂SO₄, and concentrated under reduced pressure. The residue was purified using method RPFC1 (10% to 100% gradient in 20 min), yielding 3,5-dibromo-4-(5-fluoro-2-methoxyphenyl)-4H-1,2,4-triazole (2.5 g, 69%) as a colourless solid. m/z: ES⁺ [M+H]⁺=351.85.

### Step 3: 4-(5-fluoro-2-methoxyphenyl)-3,5-divinyl-4H-1,2,4-triazole

A solution of 3,5-dibromo-4-(5-fluoro-2-methoxyphenyl)-4H-1,2,4-triazole (2.5 g, 7.1 mmol, 1.0 equiv), 2-ethenyl-4,4,5,5-tetramethyl-1,3,2-dioxaborolane (4.4 g, 29 mmol, 4.0 equiv), PdCl₂ [P(tBu)₂Ph]₂ (0.89 g, 1.4 mmol, 0.2 equiv) and K₂CO₃ (2.0 g, 14 mmol, 2.0 equiv) in toluene (25 mL)/water (2 mL) was stirred at 90° C. for 16 h under nitrogen. The resulting mixture was diluted with water and extracted with DCM. The combined organic layers were washed with brine, dried over Na₂SO₄, and concentrated under reduced pressure. The residue was purified using method RPFC1 (10% to 100% gradient in 20 min), resulting in 4-(5-fluoro-2-methoxyphenyl)-3,5-divinyl-4H-1,2,4-triazole (600 mg, 34%) as a colourless solid. m/z: ES⁺ [M+H]⁺=246.15.

### Step 4: 3,5-diethyl-4-(5-fluoro-2-methoxyphenyl)-4H-1,2,4-triazole

A solution of 4-(5-fluoro-2-methoxyphenyl)-3,5-divinyl-4H-1,2,4-triazole (600 mg, 2.5 mmol, 1.0 equiv), Pd/C (450 mg, 10% wt, wet) and AcOH (0.1 mL) in MeOH (10 mL) was stirred at room temperature for 16 h under hydrogen. The mixture was filtered through a Celite pad and concentrated under reduced pressure. This resulted in 3,5-diethyl-4-(5-fluoro-2-methoxyphenyl)-4H-1,2,4-triazole (600 mg, 98%) as a colourless solid. m/z: ES⁺ [M+H]⁺=250.00.

### Step 5: 2-(3,5-diethyl-4H-1,2,4-triazol-4-yl)-4-fluorophenol

A solution of 3,5-diethyl-4-(5-fluoro-2-methoxyphenyl)-4H-1,2,4-triazole (600 mg, 2.4 mmol, 1.0 equiv) and BBr₃ (9.6 mL, 9.6 mmol, 4.0 equiv) in DCM (3 mL) was stirred at 40° C. for 16 h. The reaction was then poured into MeOH at 0° C., and was concentrated under reduced pressure. The residue was purified using method RPFC1 (10% to 100% gradient in 20 min), resulting in 2-(3,5-diethyl-4H-1,2,4-triazol-4-yl)-4-fluorophenol (400 mg, 71%) as an off-white solid. m/z: ES⁺ [M+H]⁺=236.05.

### 2-(3,5-diisopropyl-4H-1,2,4-triazol-4-yl)-4-fluorophenol

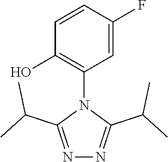 [SMILES: CC(C)c1nnc(C(C)C)n1-c1cc(F)ccc1O]

### Step 1: N-(5-fluoro-2-methoxyphenyl)isobutyramide

To a stirred solution of 5-fluoro-2-methoxyaniline (1.0 g, 7.1 mmol, 1.0 equiv) and NEt₃ (1.4 g, 14 mmol, 2.0 equiv) in DCM (10 mL) was added isobutyryl chloride (0.75 g, 7.1 mmol, 1.0 equiv) dropwise at 0° C. The resulting mixture was stirred at room temperature for 3 h under nitrogen and then extracted with DCM. The combined organic layers were washed with brine, dried over Na₂SO₄, and concentrated under reduced pressure. The residue was purified by silica gel column chromatography, eluting with PE/EtOAC (1:1) to afford N-(5-fluoro-2-methoxyphenyl)isobutyramide (1.4 g, 94%) as a yellow solid. m/z: ES⁺ [M+H]⁺=212.05.

### Step 2: N-(5-fluoro-2-methoxyphenyl)-2-methylpropanethioamide

A solution of N-(5-fluoro-2-methoxyphenyl)isobutyramide (1.4 g, 6.6 mmol, 1.0 equiv) and Lawesson's reagent (1.3 g, 3.3 mmol, 0.5 equiv) in dioxane (15 mL) was stirred at 100° C. for 16 h under nitrogen. The resulting mixture was poured into water and extracted with DCM. The combined organic layers were washed with brine, dried over anhydrous Na₂SO₄, and concentrated under reduced pressure. The residue was purified using method RPFC1 (40% to 100% gradient in 20 min) to afford N-(5-fluoro-2-methoxyphenyl)-2-methylpropanethioamide (1.2 g, 80%) as a yellow solid. m/z: ES⁺ [M+H]⁺=228.00.

### Step 3: 4-(5-fluoro-2-methoxyphenyl)-3,5-diisopropyl-4H-1,2,4-triazole

A solution of N-(5-fluoro-2-methoxyphenyl)-2-methylpropanethioamide (1.2 g, 5.3 mmol, 1.0 equiv) and isobutyrohydrazide (2.7 g, 26 mmol, 5.0 equiv) in tBuOH (12 mL) was stirred at 130° C. for 24 h under nitrogen. The resulting mixture was concentrated under reduced pressure, and the residue was purified using method RPFC1 (10% to 80% gradient in 20 min) to afford 4-(5-fluoro-2-methoxyphenyl)-3,5-diisopropyl-4H-1,2,4-triazole (1.0 g, 68%) as a light yellow solid. m/z: ES⁺ [M+H]⁺=278.00.

### Step 4: 2-(3,5-diisopropyl-4H-1,2,4-triazol-4-yl)-4-fluorophenol

A solution of 4-(5-fluoro-2-methoxyphenyl)-3,5-diisopropyl-4H-1,2,4-triazole (1.0 g, 3.6 mmol, 1.0 equiv) and BBr₃ (1 M in DCM, 14 mL, 14 mmol, 4.0 equiv) in DCM (3 mL) was stirred at room temperature for 16 h. The reaction was poured into MeOH at 0° C. The resulting mixture was concentrated under reduced pressure. The residue was purified using method RPFC1 (10% to 70% gradient in 20 min) to afford 2-(3,5-diisopropyl-4H-1,2,4-triazol-4-yl)-4-fluorophenol (600 mg, 63%) as a colourless solid. m/z: ES⁺ [M+H]⁺=264.00.

### 2-(3,5-diisopropyl-1-(tetrahydro-2H-pyran-2-yl)-1H-pyrazol-4-yl)-4-fluorophenol

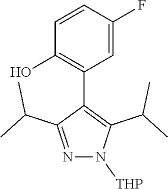

### Step 1: 4-bromo-3,5-diisopropyl-1-(tetrahydro-2H-pyran-2-yl)-1H-pyrazole

A mixture of 4-bromo-3,5-diisopropyl-1H-pyrazole (500 mg, 0.087 mmol, 1.0 equiv), DHP (320 mg, 0.17 mmol, 2.0 equiv) and TsOH (160 mg, 0.043 mmol, 0.5 equiv) in THF (6 mL) was stirred at 50° C. for 16 h. The resulting mixture was diluted with water (50 mL) and extracted with EtOAc (3×5 mL), dried over Na₂SO₄, and concentrated under reduced pressure. The residue was purified using method RPFC1 (10% to 100% gradient in 20 min) to afford 4-bromo-3,5-diisopropyl-1-(tetrahydro-2H-pyran-2-yl)-1H-pyrazole (460 mg, 51%) as a yellow solid. m/z: ES⁺ [M+H]⁺=315.10.

### Step 2: 4-(2-(benzyloxy)-5-fluorophenyl)-3,5-diisopropyl-1-(tetrahydro-2H-pyran-2-yl)-1H-pyrazole

A mixture of 4-bromo-3,5-diisopropyl-1-(tetrahydro-2H-pyran-2-yl)-1H-pyrazole (450 mg, 1.4 mmol, 1.0 equiv), (2-(benzyloxy)-5-fluorophenyl)boronic acid (1.1 g, 4.3 mmol, 3.0 equiv), Pd(PPh₃)₄ (165 mg, 0.14 mmol, 0.1 equiv) and K₃PO₄ (606 mg, 2.9 mmol, 2.0 equiv) in dioxane (10 mL) and water (2 mL) was stirred at 100° C. 16 h under nitrogen. The resulting mixture was diluted with water (10 mL) and extracted with EtOAc (3×10 mL), dried over Na₂SO₄, and concentrated under reduced pressure. The residue was purified using method RPFC1 (10% to 100% gradient in 20 min) to afford 4-(2-(benzyloxy)-5-fluorophenyl)-3,5-diisopropyl-1-(tetrahydro-2H-pyran-2-yl)-1H-pyrazole (320 mg, 51%) as a brown yellow semi-solid. m/z: ES⁺ [M+H]⁺=437.25.

### Step 3: 2-(3,5-diisopropyl-1-(tetrahydro-2H-pyran-2-yl)-1H-pyrazol-4-yl)-4-fluorophenol

A mixture of 4-(2-(benzyloxy)-5-fluorophenyl)-3,5-diisopropyl-1-(tetrahydro-2H-pyran-2-yl)-1H-pyrazole (300 mg, 0.69 mmol, 1.0 equiv) and Pd/C (50 mg, 10% wt, wet) in MeOH (4 mL) was stirred at room temperature for 24 h under hydrogen. The resulting mixture was filtered, the filter cake was washed with MeOH (5×5 mL). The filtrate was concentrated under reduced pressure to afford 2-(3,5-diisopropyl-1-(tetrahydro-2H-pyran-2-yl)-1H-pyrazol-4-yl)-4-fluorophenol (170 mg, 71%) as a colourless solid. m/z: ES⁺ [M+H]⁺=347.21.

### 2-(5-cyclopropyl-2,4-dimethyl-1H-imidazol-1-yl)-4-fluorophenol

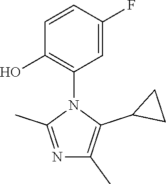 [SMILES: Cc1nc(C)n(-c2cc(F)ccc2O)c1C1CC1]

### Step 1: 1-(2-(benzyloxy)-5-fluorophenyl)-2,4-dimethyl-1H-imidazole

A solution of (2-(benzyloxy)-5-fluorophenyl)boronic acid (2.0 g, 8.1 mmol, 1.0 equiv) and 2,4-dimethyl-1H-imidazole (0.94 g, 9.8 mmol, 1.2 equiv) and Cu(OAc)₂ (0.74 g, 4.1 mmol, 0.5 equiv) and NEt₃ (2.5 g, 24 mmol, 3.0 equiv) in MeOH (20 mL) was stirred at room temperature for 24 h under air. After filtration, the filtrate was concentrated under reduced pressure, and the residue purified using method RPFC1 (10% to 100% gradient in 30 min), resulting in 1-(2-(benzyloxy)-5-fluorophenyl)-2,4-dimethyl-1H-imidazole (400 mg, 17%) as a brown semi-solid. m/z: ES⁺ [M+H]⁺=297.05.

### Step 2: 1-(2-(benzyloxy)-5-fluorophenyl)-5-bromo-2,4-dimethyl-1H-imidazole

A solution of 1-(2-(benzyloxy)-5-fluorophenyl)-2,4-dimethyl-1H-imidazole (400 mg, 1.4 mmol, 1.0 equiv) and NBS (216 mg, 1.2 mmol, 0.9 equiv) in DMF (4 mL) was stirred at room temperature for 1 h. The resulting mixture was then diluted with water and extracted with EtOAc. The combined organic layers were washed with brine, dried over Na₂SO₄, and was concentrated under reduced pressure. The residue was purified using method RPFC1 (10% to 100% gradient in 20 min), resulting in 1-(2-(benzyloxy)-5-fluorophenyl)-5-bromo-2,4-dimethyl-1H-imidazole (250 mg, 49%) as a brown yellow semi-solid. m/z: ES⁺ [M+H]⁺=375.00.

### Step 3: 1-(2-(benzyloxy)-5-fluorophenyl)-5-cyclopropyl-2,4-dimethyl-1H-imidazole

A solution of 1-(2-(benzyloxy)-5-fluorophenyl)-5-bromo-2,4-dimethyl-1H-imidazole (250 mg, 0.67 mmol, 1.0 equiv), cyclopropylboronic acid (114 mg, 1.3 mmol, 2.0 equiv), PdCl₂ [P(tBu)₂Ph]₂ (62 mg, 0.1 mmol, 0.15 equiv), Ag₂O (309 mg, 1.3 mmol, 2.0 equiv) and K₂CO₃ (276 mg, 2.0 mmol, 3.0 equiv) in dioxane (5 mL) was stirred at 90° C. overnight under nitrogen. After filtration, the filtrate was concentrated under reduced pressure, and the residue was purified using method RPFC1 (0% to 100% gradient in 30 min), resulting in 1-(2-(benzyloxy)-5-fluorophenyl)-5-cyclopropyl-2,4-dimethyl-1H-imidazole (110 mg, 49%) as a brown semi-solid. m/z: ES⁺ [M+H]⁺=337.10.

### Step 4: 2-(5-cyclopropyl-2,4-dimethyl-1H-imidazol-1-yl)-4-fluorophenol

A solution of 1-(2-(benzyloxy)-5-fluorophenyl)-5-cyclopropyl-2,4-dimethyl-1H-imidazole (110 mg, 0.33 mmol, 1.0 equiv) and Pd/C (50 mg, 10% wt, wet) in MeOH (2 mL) was stirred at room temperature for 16 h under hydrogen. The resulting mixture was then filtered; the filter cake was washed with MeOH (3×10 mL), and the filtrate was concentrated under reduced pressure. This resulted in 2-(5-cyclopropyl-2,4-dimethyl-TH-imidazol-1-yl)-4-fluorophenol (70 mg, 87%) as a yellow semi-solid. m/z: ES⁺ [M+H]⁺=247.10.

### 4-fluoro-2-(1-isopropyl-4-methyl-1H-1,2,3-triazol-5-yl)phenol

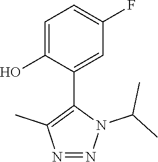 [SMILES: Cc1nnn(C(C)C)c1-c1cc(F)ccc1O]

### Step 1: 5-iodo-1-isopropyl-4-methyl-1H-1,2,3-triazole

To a stirred mixture of 1-iodoprop-1-yne (2.0 g, 12 mmol, 1.0 equiv) and 2-azidopropane (1.0 g, 12 mmol, 1.0 equiv) in MeCN (30 mL) were added CuI (0.23 g, 1.2 mmol, 0.1 equiv) and NEt₃ (3.7 g, 36 mmol, 3.0 equiv) at room temperature under nitrogen. The resulting mixture was stirred at room temperature for 12 h under nitrogen, and then filtered. The filter cake was washed with MeCN (3×20 mL), and the filtrate was concentrated under reduced pressure. The residue was purified using method RPFC1 (0% to 60% gradient in 20 min) to afford 5-iodo-1-isopropyl-4-methyl-1H-1,2,3-triazole (450 mg, 15%) as an off-white solid. m/z: ES⁺ [M+H]⁺=251.95.

### Step 2: 5-(2-(benzyloxy)-5-fluorophenyl)-1-isopropyl-4-methyl-1H-1,2,3-triazole

To a stirred mixture of 5-iodo-1-isopropyl-4-methyl-TH-1,2,3-triazole (440 mg, 1.75 mmol, 1.0 equiv) and 2-(benzyloxy)-5-fluorophenylboronic acid (1.3 g, 5.2 mmol, 3.0 equiv) in dioxane (8 mL) were added Pd(dppf)Cl₂ (128 mg, 0.18 mmol, 0.1 equiv) and K₂CO₃ (726 mg, 5.2 mmol, 3.0 equiv) at room temperature under nitrogen. The resulting mixture was stirred at 100° C. for 24 h under nitrogen. Water was then added and the mixture was extracted with EtOAc (3×30 mL). The combined organic layers were washed with brine (2×10 mL), dried over Na₂SO₄, and concentrated under reduced pressure. The residue was purified by Prep-TLC (DCM/MeOH 10:1) to afford 5-(2-(benzyloxy)-5-fluorophenyl)-1-isopropyl-4-methyl-TH-1,2,3-triazole (300 mg, 53%) as a yellow solid. m/z: ES⁺ [M+H]⁺=326.10.

### Step 3: 4-fluoro-2-(1-isopropyl-4-methyl-1H-1,2,3-triazol-5-yl)phenol

A mixture of 5-[2-(benzyloxy)-5-fluorophenyl]-1-isopropyl-4-methyl-1,2,3-triazole (300 mg, 0.92 mmol, 1.0 equiv) and Pd/C (100 mg, 10% wt, wet) in MeOH (5 mL) was stirred at room temperature for 2 h under hydrogen. The resulting mixture was filtered, the filter cake was washed with MeOH (3×10 mL), and the filtrate was concentrated under reduced pressure to afford 4-fluoro-2-(1-isopropyl-4-methyl-1H-1,2,3-triazol-5-yl)phenol (170 mg, 78%) as a colourless solid. m/z: ES⁺ [M+H]⁺=236.10.

### 4-fluoro-2-(1-isopropyl-4-methyl-1H-pyrazol-5-yl)phenol

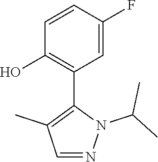 [SMILES: Cc1cnn(C(C)C)c1-c1cc(F)ccc1O]

### Step 1: 5-(2-(benzyloxy)-5-fluorophenyl)-1-isopropyl-1H-pyrazole

A mixture of (2-(benzyloxy)-5-fluorophenyl)boronic acid (2.0 g, 7.9 mmol, 1.5 equiv), 5-bromo-1-isopropyl-1H-pyrazole (1.0 g, 5.3 mmol, 1.0 equiv), Pd(dppf)Cl₂CH₂Cl₂ (0.86 g, 1.1 mmol, 0.2 equiv) and K₂CO₃ (2.2 g, 16 mmol, 3.0 equiv) in dioxane (20 mL) and water (4 mL) was stirred at 100° C. for 4 h under nitrogen. The resulting mixture was diluted with water (100 mL) and extracted with EtOAc (3×100 mL), dried over Na₂SO₄, and concentrated under reduced pressure. The crude product was purified using method RPFC1 (10% to 100% gradient in 20 min) to afford 5-(2-(benzyloxy)-5-fluorophenyl)-1-isopropyl-1H-pyrazole (1.6 g, 97%) as a brown solid. m/z: ES⁺ [M+H]⁺=311.15.

### Step 2: 5-(2-(benzyloxy)-5-fluorophenyl)-4-bromo-1-isopropyl-1H-pyrazole

A mixture of 5-(2-(benzyloxy)-5-fluorophenyl)-1-isopropyl-1H-pyrazole (1.6 g, 5.2 mmol, 1.0 equiv) and NBS (0.92 g, 5.2 mmol, 1.0 equiv) in DMF (10 mL) was stirred at room temperature for 2 h. The resulting mixture was then diluted with water (100 mL) and extracted with EtOAc (3×100 mL), dried over Na₂SO₄, and concentrated under reduced pressure. The crude product was purified using method RPFC1 (10% to 100% gradient in 20 min) to afford 5-(2-(benzyloxy)-5-fluorophenyl)-4-bromo-1-isopropyl-1H-pyrazole (1.6 g, 80%) as a brown solid. m/z: ES⁺ [M+H]⁺=389.10.

### Step 3: 5-(2-(benzyloxy)-5-fluorophenyl)-1-isopropyl-4-methyl-1H-pyrazole

A mixture of 5-(2-(benzyloxy)-5-fluorophenyl)-4-bromo-1-isopropyl-1H-pyrazole (1.6 g, 4.1 mmol, 1.0 equiv), methylboronic acid (0.49 g, 8.2 mmol, 2.0 equiv), Pd₂(dba)₃ (0.75 g, 0.82 mmol, 0.2 equiv), XPhos (0.39 g, 0.82 mmol, 0.2 equiv) and tBuONa (1.2 g, 12 mmol, 3.0 equiv) in dioxane (10 mL) was stirred at 100° C. for overnight under nitrogen. The resulting mixture was then diluted with water (30 mL) and extracted with EtOAc (3×30 mL), dried over Na₂SO₄, and concentrated under reduced pressure. The crude product was purified using method RPFC1 (10% to 100% gradient in 20 min) to afford 5-(2-(benzyloxy)-5-fluorophenyl)-1-isopropyl-4-methyl-1H-pyrazole (700 mg, 53%) as a brown oil. m/z: ES⁺ [M+H]⁺=325.10.

### Step 4: 4-fluoro-2-(1-isopropyl-4-methyl-1H-pyrazol-5-yl)phenol

To a solution of 5-(2-(benzyloxy)-5-fluorophenyl)-1-isopropyl-4-methyl-1H-pyrazole (700 mg, 2.2 mmol, 1.0 equiv) in 10 mL EtOAc was added Pd/C (10% wt, 300 mg, wet) under nitrogen. The mixture was stirred at room temperature for 4 h under hydrogen, and then filtered through a Celite pad and concentrated under reduced pressure to afford 4-fluoro-2-(1-isopropyl-4-methyl-1H-pyrazol-5-yl)phenol (500 mg, 99%) as a light yellow oil. m/z: ES⁺ [M+H]⁺=235.05.

### 4-fluoro-2-(5-(1-hydroxycyclopropyl)-2-methyl-1H-imidazol-1-yl)phenol

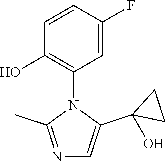 [SMILES: Cc1ncc(C2(O)CC2)n1-c1cc(F)ccc1O]

### Step 1: 1-(1-(2-(benzyloxy)-5-fluorophenyl)-2-methyl-1H-imidazol-5-yl)cyclopropan-1-ol

To a stirred solution of methyl 1-(2-(benzyloxy)-5-fluorophenyl)-2-methyl-1H-imidazole-5-carboxylate (1.0 g, 2.9 mmol, 1.0 equiv) in THF (10 mL) was added Ti(EtO)₄ (1.3 g, 5.9 mmol, 2.0 equiv) and EtMgBr (1.0 M in THF, 8.8 mL, 8 mmol) in portions at 0° C. under nitrogen. The resulting mixture was stirred at room temperature for 12 h under nitrogen, and then quenched with sat NH₄Cl at 0° C. The resulting mixture was filtered, the filter cake was washed with MeOH (3×10 mL), and the filtrate was concentrated under reduced pressure. The residue was purified using method RPFC4 (0% to 100% gradient in 20 min) to give 1-(1-(2-(benzyloxy)-5-fluorophenyl)-2-methyl-1H-imidazol-5-yl)cyclopropan-1-ol (485 mg, 49%) as alight yellow oil. m/z: ES⁺ [M+H]⁺=339.15.

### Step 2: 4-fluoro-2-(5-(1-hydroxycyclopropyl)-2-methyl-1H-imidazol-1-yl)phenol

A solution of 1-(1-(2-(benzyloxy)-5-fluorophenyl)-2-methyl-1H-imidazol-5-yl)cyclopropan-1-ol (150 mg, 0.44 mmol, 1.0 equiv) and Pd/C (50 mg, 10% wt, wet) in MeOH (6 mL) was stirred at room temperature for 2 h under hydrogen. The resulting mixture was filtered and the filter cake was washed with MeOH (6×10 mL). The filtrate was concentrated under reduced pressure to afford 4-fluoro-2-(5-(1-hydroxycyclopropyl)-2-methyl-1H-imidazol-1-yl)phenol (80 mg, 73%) as a colourless solid. m/z: ES⁺ [M+H]⁺=249.15.

### 4-fluoro-2-(5-(1-fluorocyclopropyl)-2-methyl-1H-imidazol-1-yl)phenol

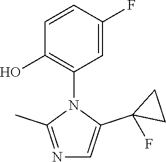 [SMILES: Cc1ncc(C2(F)CC2)n1-c1cc(F)ccc1O]

### Step 1: 1-(2-(benzyloxy)-5-fluorophenyl)-5-(1-fluorocyclopropyl)-2-methyl-1H-imidazole

A solution of 1-(1-(2-(benzyloxy)-5-fluorophenyl)-2-methyl-1H-imidazol-5-yl)cyclopropan-1-ol (200 mg, 0.59 mmol, 1.0 equiv) and DAST (190 mg, 1.2 mmol, 2.0 equiv) in DCM (8 mL) was stirred at −78° C. under nitrogen for 1.5 h. The reaction was quenched with sat. NaHCO₃ at room temperature and extracted with EtOAc (2×40 mL). The combined organic layers were washed with brine (80 mL), dried over Na₂SO₄, and concentrated under reduced pressure. The residue was purified using method RPFC1 (20% to 80% gradient in 25 min) to give 1-(2-(benzyloxy)-5-fluorophenyl)-5-(1-fluorocyclopropyl)-2-methyl-1H-imidazole (110 mg, 55%) as a light yellow oil. m/z: ES⁺ [M+H]⁺=341.15.

### Step 2: 4-fluoro-2-(5-(1-fluorocyclopropyl)-2-methyl-1H-imidazol-1-yl)phenol

A solution of 1-(2-(benzyloxy)-5-fluorophenyl)-5-(1-fluorocyclopropyl)-2-methyl-1H-imidazole (100 mg, 0.29 mmol, 1.0 equiv) and Pd/C (31 mg, 0.29 mmol, 1.0 equiv) in MeOH (8 mL) was stirred at room temperature for 2 h under hydrogen. The resulting mixture was filtered and the filter cake was washed with MeOH (3×10 mL). The filtrate was concentrated under reduced pressure to afford 4-fluoro-2-(5-(1-fluorocyclopropyl)-2-methyl-1H-imidazol-1-yl)phenol (70 mg, 95%) as a colourless solid. m/z: ES⁺ [M+H]⁺=251.20.

### 2-(4-cyclopropyl-1-methyl-1H-pyrazol-5-yl)-4-fluorophenol

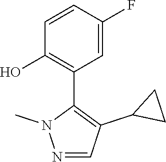 [SMILES: Cn1ncc(C2CC2)c1-c1cc(F)ccc1O]

### Step 1: 5-(2-(benzyloxy)-5-fluorophenyl)-1-methyl-1H-pyrazole

A solution of (2-(benzyloxy)-5-fluorophenyl)boronic acid (500 mg, 2.0 mmol, 1.0 equiv), 5-bromo-1-methyl-1H-pyrazole (0.39 g, 2.4 mmol, 1.2 equiv), Pd(dppf)Cl₂CH₂Cl₂ (0.16 g, 0.2 mmol, 0.1 equiv) and K₂CO₃ (0.84 g, 6.1 mmol, 3.0 equiv) in dioxane (5 mL) and water (1 mL) was stirred at 100° C. under nitrogen for 16 h. The resulting mixture was then diluted with water (20 mL) and extracted with EtOAc (2×30 mL). The combined organic layers were washed with brine (60 mL), dried over Na₂SO₄, and concentrated under reduced pressure. The residue was purified using method RPFC1 (10% to 100% gradient in 20 min) to give 5-(2-(benzyloxy)-5-fluorophenyl)-1-methyl-1H-pyrazole (550 mg, 96%) as a brown yellow oil. m/z: ES⁺ [M+H]⁺=283.12.

### Step 2: 5-(2-(benzyloxy)-5-fluorophenyl)-4-bromo-1-methyl-1H-pyrazole

A solution of 5-(2-(benzyloxy)-5-fluorophenyl)-1-methyl-1H-pyrazole (500 mg, 1.7 mmol, 1.0 equiv) and NBS (0.34 g, 1.9 mmol, 1.1 equiv) in MeCN (5 mL) was stirred at 25° C. for 2 h. The resulting mixture was then diluted with water (20 mL) and extracted with EtOAc (2×30 mL). The combined organics were washed with brine (60 mL), dried over Na₂SO₄, and concentrated under reduced pressure. The residue was purified using method RPFC1 (10% to 90% gradient in 25 min) to give 5-(2-(benzyloxy)-5-fluorophenyl)-4-bromo-1-methyl-1H-pyrazole (550 mg, 96%) as a colourless solid. m/z: ES⁺ [M+H]⁺=361.03.

### Step 3: 5-(2-(benzyloxy)-5-fluorophenyl)-4-cyclopropyl-1-methyl-1H-pyrazole

A solution of 5-(2-(benzyloxy)-5-fluorophenyl)-4-bromo-1-methyl-1H-pyrazole (500 mg, 1.4 mmol, 1.0 equiv), cyclopropylboronic acid (356 mg, 4.2 mmol, 3.0 equiv), K₂CO₃ (570 mg, 4.2 mmol, 3.0 equiv) and Pd(dppf)Cl₂CH₂Cl₂ (110 mg, 0.14 mmol, 0.1 equiv) in dioxane (7 mL) and water (1 mL) was stirred at 100° C. for 16 h under nitrogen. The resulting mixture was diluted with water (20 mL) and extracted with EtOAc (2×30 mL). The combined organic layers were washed with brine (60 mL), dried over anhydrous Na₂SO₄, and concentrated under reduced pressure. The residue was purified using method RPFC1 (10% to 90% gradient in 30 min) to give 5-(2-(benzyloxy)-5-fluorophenyl)-4-cyclopropyl-1-methyl-1H-pyrazole (400 mg, 90%) as a yellow solid. m/z: ES⁺ [M+H]⁺=323.15.

### Step 4: 2-(4-cyclopropyl-1-methyl-1H-pyrazol-5-yl)-4-fluorophenol

A solution of 5-(2-(benzyloxy)-5-fluorophenyl)-4-cyclopropyl-1-methyl-1H-pyrazole (400 mg, 1.2 mmol, 1.0 equiv) and Pd/C (200 mg) in EtOAc (4 mL) was stirred at 25° C. for 6 h under hydrogen. The resulting mixture was filtered, the filter cake was washed with EtOAc. The filtrate was concentrated under reduced pressure and the crude product was used in the next step directly without further purification. m/z: ES⁺ [M+H]⁺=233.20.

### 4-fluoro-2-(4-isopropyl-1-methyl-1H-pyrazol-5-yl)phenol

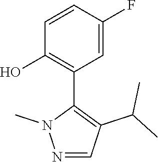 [SMILES: CC(C)c1cnn(C)c1-c1cc(F)ccc1O]

### Step 1: 5-(2-(benzyloxy)-5-fluorophenyl)-1-methyl-4-(prop-1-en-2-yl)-1H-pyrazole

A solution of 5-(2-(benzyloxy)-5-fluorophenyl)-4-bromo-1-methyl-1H-pyrazole (500 mg, 1.4 mmol, 1.0 equiv), 4,4,5,5-tetramethyl-2-(prop-1-en-2-yl)-1,3,2-dioxaborolane (470 mg, 2.8 mmol, 2.0 equiv), K₃PO₄ (880 mg, 4.2 mmol, 3.0 equiv) and Pd(PPh₃)₄ (320 mg, 0.28 mmol, 0.2 equiv) in dioxane (7 mL) and water (1 mL) was stirred at 100° C. for 16 h under nitrogen. The resulting mixture was filtered and the filter cake was washed with EtOAc. The filtrate was concentrated under reduced pressure and the residue purified using method RPFC1 (10% to 80% gradient in 20 min) to give 5-(2-(benzyloxy)-5-fluorophenyl)-1-methyl-4-(prop-1-en-2-yl)-1H-pyrazole (350 mg, 78%) as a yellow solid. m/z: ES⁺ [M+H]⁺=323.15.

### Step 2: 4-fluoro-2-(4-isopropyl-1-methyl-1H-pyrazol-5-yl)phenol

A solution of 5-(2-(benzyloxy)-5-fluorophenyl)-1-methyl-4-(prop-1-en-2-yl)-1H-pyrazole (350 mg, 1.1 mmol, 1.0 equiv) and Pd/C (200 mg, 10% wt, wet) in EtOAc (4 mL) was stirred at 25° C. for 6 h under hydrogen. The resulting mixture was filtered and the filter cake washed with EtOAc. The filtrate was concentrated under reduced pressure and the crude product used in the next step directly without further purification. m/z: ES⁺ [M+H]⁺=235.15.

### 4-fluoro-2-(4-isopropyl-1-methyl-1H-imidazol-5-yl)phenol

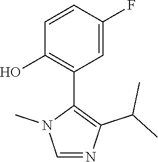 [SMILES: CC(C)c1ncn(C)c1-c1cc(F)ccc1O]

### Step 1: 5-(2-(benzyloxy)-5-fluorophenyl)-4-isopropyl-1-methyl-1H-imidazole

A mixture of 5-bromo-4-isopropyl-1-methyl-1H-imidazole (1.0 g, 4.9 mmol, 1.0 equiv), (2-(benzyloxy)-5-fluorophenyl)boronic acid (2.4 g, 9.8 mmol, 2.0 equiv), Pd(dppf)Cl₂CH₂Cl₂ (0.80 g, 1.0 mmol, 0.2 equiv) and K₂CO₃ (2.0 g, 15 mmol, 3.0 equiv) in dioxane (20 mL) and water (4 mL) was stirred at 100° C. for 16 h under nitrogen. The resulting mixture was then diluted with water (100 mL), and extracted with EtOAc (3×100 mL), dried over Na₂SO₄, and concentrated under reduced pressure. The crude product was purified using method RPFC1 (10% to 100% gradient in 20 min) to give 5-(2-(benzyloxy)-5-fluorophenyl)-4-isopropyl-1-methyl-1H-imidazole (200 mg, 13%) as a brown oil. m/z: ES⁺ [M+H]⁺=325.20.

### Step 2: 4-fluoro-2-(4-isopropyl-1-methyl-1H-imidazol-5-yl)phenol

A solution of 5-(2-(benzyloxy)-5-fluorophenyl)-4-isopropyl-1-methyl-1H-imidazole (200 mg, 0.17 mmol, 1.0 equiv) and Pd/C (1.3 g, 10% wt, wet) in EtOAc (25 mL) was stirred at 25° C. for 16 h under hydrogen. The resulting mixture was filtered through a Celite pad and concentrated under reduced pressure to afford 4-fluoro-2-(4-isopropyl-1-methyl-1H-imidazol-5-yl)phenol (100 mg, 69%) as a yellow oil. m/z: ES⁺ [M+H]⁺=235.10.

### 4-fluoro-2-(5-isopropyl-3-methylisoxazol-4-yl)phenol

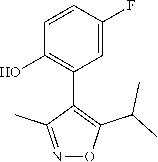 [SMILES: Cc1noc(C(C)C)c1-c1cc(F)ccc1O]

### Step 1: 5-isopropyl-3-methylisoxazole and 3-isopropyl-5-methylisoxazole

To a stirred solution of 5-methylhexane-2,4-dione (1.8 g, 14 mmol, 1.0 equiv) in EtOH (20 mL) was added hydroxylamine hydrochloride (2.0 g, 28 mmol, 2.0 equiv) and the resulting mixture was stirred at 80° C. overnight then basified to pH 9 with sat. NaHCO₃ and extracted with DCM (2×100 mL). The combined organic layers were washed with brine (2×200 mL), dried over Na₂SO₄, and concentrated under reduced pressure to afford a mixture of 5-isopropyl-3-methylisoxazole and 3-isopropyl-5-methylisoxazole (1.6 g, 82%) as a light yellow oil which was used directly in the next step. m/z: ES⁺ [M+H]⁺=126.10.

### Step 2: 4-bromo-5-isopropyl-3-methylisoxazole and 4-bromo-3-isopropyl-5-methyl isoxazole

A solution of 5-isopropyl-3-methylisoxazole and 3-isopropyl-5-methylisoxazole (1.6 g, 13 mmol, 1.0 equiv) and NBS (3.4 g, 19 mmol, 1.5 equiv) in DMF (20 mL) was stirred at room temperature for 2 h. The resulting mixture was diluted with water (100 mL), and extracted with DCM (2×100 mL). The combined organic layers were washed with brine (2×200 mL), dried over Na₂SO₄, and concentrated under reduced pressure to afford a mixture of 4-bromo-5-isopropyl-3-methylisoxazole and 4-bromo-3-isopropyl-5-methylisoxazole (2.5 g, 96%) as a brown oil which was used directly in the next step. m/z: ES⁺ [M+H]⁺=206.00.

### Step 3: 4-(2-(benzyloxy)-5-fluorophenyl)-5-isopropyl-3-methylisoxazole and 4-(2-(benzyloxy)-5-fluorophenyl)-3-isopropyl-5-methylisoxazole

To a stirred solution of 4-bromo-5-isopropyl-3-methylisoxazole and 4-bromo-3-isopropyl-5-methylisoxazole (2.4 g, 12 mmol, 1.0 equiv) and 2-(benzyloxy)-5-fluorophenylboronic acid (3.5 g, 14 mmol, 1.2 equiv) in toluene (30 mL) and water (3 mL) were added PdCl₂(dcypf) (1.8 g, 2.4 mmol, 0.2 equiv) and K₂CO₃ (3.3 g, 24 mmol, 2.0 equiv). The resulting mixture was stirred at 100° C. for 2 h under nitrogen, and then diluted with water (100 mL), and extracted with DCM (2×100 mL). The combined organic layers were washed with brine (2×200 mL), dried over Na₂SO₄, and concentrated under reduced pressure to afford a mixture of 4-(2-(benzyloxy)-5-fluorophenyl)-5-isopropyl-3-methylisoxazole and 4-(2-(benzyloxy)-5-fluorophenyl)-3-isopropyl-5-methylisoxazole) (1.7 g, 44%) as a light brown oil which was used directly in the next step. m/z: ES⁺ [M+H]⁺=326.30.

### Step 4: 4-fluoro-2-(5-isopropyl-3-methylisoxazol-4-yl)phenol

To a solution of 4-(2-(benzyloxy)-5-fluorophenyl)-5-isopropyl-3-methylisoxazole and 4-(2-(benzyloxy)-5-fluoro phenyl)-3-isopropyl-5-methylisoxazole (1.4 g, 4.3 mmol, 1.0 equiv) in EtOAc (15 mL) was added Pd/C (10% wt, 92 mg, wet) under nitrogen. The mixture was stirred under hydrogen for 16 h, then filtered through a Celite pad and concentrated under reduced pressure. The residue was purified using method RPFC1 (10% to 50% gradient in 10 min) to afford a mixture of 4-fluoro-2-(5-isopropyl-3-methylisoxazol-4-yl)phenol and 4-fluoro-2-(3-isopropyl-5-methylisoxazol-4-yl)phenol (1.0 g, 99%) as a colourless oil, which were separated using method CHIRALPAKIG (Gradient 15% B to 45% B in A where Mobile Phase A: Hex(0.5% 2M NH₃-MeOH), Mobile Phase B: EtOH/Hex=1/9; RT1(min): 15.7; RT2(min): 18.8) to afford 4-fluoro-2-(5-isopropyl-3-methylisoxazol-4-yl)phenol (600 mg, 60%, peak 1) as a colourless solid. m/z: ES⁺ [M+H]⁺=236.00.

### 2-(1-cyclopropyl-4-methyl-1H-pyrazol-5-yl)-4-fluorophenol

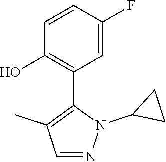 [SMILES: Cc1cnn(C2CC2)c1-c1cc(F)ccc1O]

### Step 1: 5-(2-(benzyloxy)-5-fluorophenyl)-1-cyclopropyl-1H-pyrazole

A solution of (2-(benzyloxy)-5-fluorophenyl)boronic acid (1.0 g, 4.1 mmol, 1.0 equiv), 5-bromo-1-cyclopropyl-1H-pyrazole (0.68 g, 3.7 mmol, 0.9 equiv), Pd(dppf)Cl₂CH₂Cl₂ (664 mg, 0.81 mmol, 0.2 equiv) and K₂CO₃ (1.7 g, 12 mmol, 3.0 equiv) in dioxane (10 mL) and water (2 mL) was stirred at 100° C. under nitrogen for 16 h. The resulting mixture was then diluted with water (40 mL) and extracted with EtOAc (2×30 mL). The combined organic layers were washed with water (60 mL), dried over Na₂SO₄, and concentrated under reduced pressure. The residue was purified using method RPFC1 (20% to 90% gradient in 30 min) to give 5-(2-(benzyloxy)-5-fluorophenyl)-1-cyclopropyl-1H-pyrazole (1.0 g, 80%) as a brown yellow oil. m/z: ES⁺ [M+H]⁺=309.20.

### Step 2: 5-(2-(benzyloxy)-5-fluorophenyl)-4-bromo-1-cyclopropyl-1H-pyrazole

A solution of 5-(2-(benzyloxy)-5-fluorophenyl)-1-cyclopropyl-1H-pyrazole (1.0 g, 3.2 mmol, 1.0 equiv) and NBS (520 mg, 2.9 mmol, 0.9 equiv) in DMF (10 mL) was stirred at 25° C. for 16 h. The resulting mixture was then diluted with water (50 mL) and extracted with EtOAc (3×50 mL). The combined organics were washed with water (2×100 mL), dried over Na₂SO₄, and concentrated under reduced pressure. The residue purified using method RPFC1 (30% to 95% gradient in 25 min) to give 5-(2-(benzyloxy)-5-fluorophenyl)-4-bromo-1-cyclopropyl-1H-pyrazole (900 mg, 72%) as a brown oil. m/z: ES⁺ [M+H]⁺=387.20.

### Step 3: 5-(2-(benzyloxy)-5-fluorophenyl)-1-cyclopropyl-4-methyl-1H-pyrazole

A solution of 5-(2-(benzyloxy)-5-fluorophenyl)-4-bromo-1-cyclopropyl-1H-pyrazole (500 mg, 1.3 mmol, 1.0 equiv), methylboronic acid (232 mg, 3.9 mmol, 3.0 equiv), K₂CO₃ (570 mg, 4.2 mmol, 3.0 equiv) and Pd₂(dba)₃ (237 mg, 0.26 mmol, 0.2 equiv) in dioxane (3 mL) was stirred at 100° C. for 16 h under nitrogen. The resulting mixture was diluted with water (30 mL) and extracted with EtOAc (3×30 mL). The combined organic layers were washed with water (2×50 mL) and brine (50 mL), dried over anhydrous Na₂SO₄, and concentrated under reduced pressure. The residue was purified using method RPFC1 (10% to 50% gradient in 10 min) to give 5-(2-(benzyloxy)-5-fluorophenyl)-1-cyclopropyl-4-methyl-1H-pyrazole (100 mg, 24%) as a yellow oil. m/z: ES⁺ [M+H]⁺=323.25.

### Step 4: 2-(1-cyclopropyl-4-methyl-1H-pyrazol-5-yl)-4-fluorophenol

A solution of 5-(2-(benzyloxy)-5-fluorophenyl)-1-cyclopropyl-4-methyl-1H-pyrazole (350 mg, 1.1 mmol, 1.0 equiv) and Pd/C (350 mg, 10% wt, wet) in EtOAc (5 mL) was stirred at 25° C. for 2 h under hydrogen. The resulting mixture was filtered, the filter cake was washed with EtOAc (3×5 mL). The filtrate was concentrated under reduced pressure and the crude product was used in the next step directly without further purification. m/z: ES⁺ [M+H]⁺=233.20.

### 4-fluoro-2-(1,3,4-trimethyl-1H-pyrazol-5-yl)phenol

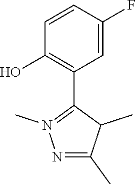

### Step 1: 5-(2-(benzyloxy)-5-fluorophenyl)-1,3-dimethyl-1H-pyrazole

A mixture of (2-(benzyloxy)-5-fluorophenyl)boronic acid (5.0 g, 20 mmol, 1.0 equiv), 5-bromo-1,3-dimethyl-1H-pyrazole (7.1 g, 41 mmol, 2.0 equiv), Pd(dppf)Cl₂CH₂Cl₂ (3.3 g, 4.1 mmol, 0.2 equiv) and K₂CO₃ (8.4 g, 61 mmol, 3.0 equiv) in dioxane (50 mL) and water (10 mL) was stirred at 100° C. for 16 h under nitrogen. The resulting mixture was then diluted with water (100 mL) and extracted with EtOAc (3×100 mL), dried over Na₂SO₄, and concentrated under reduced pressure. The crude product was purified using method RPFC1 (10% to 100% gradient in 20 min) to afford 5-(2-(benzyloxy)-5-fluorophenyl)-1,3-dimethyl-1H-pyrazole (4.0 g, 66%) as a brown solid. m/z: ES⁺ [M+H]⁺=297.25.

### Step 2: 5-(2-(benzyloxy)-5-fluorophenyl)-4-bromo-1,3-dimethyl-1H-pyrazole

A mixture of 5-(2-(benzyloxy)-5-fluorophenyl)-1,3-dimethyl-1H-pyrazole (2.8 g, 9.4 mmol, 1.0 equiv) and NBS (1.4 g, 7.6 mmol, 0.8 equiv) in DMF (20 mL) was stirred at room temperature for 16 h under nitrogen. The resulting mixture then was diluted with water (100 mL) and extracted with EtOAc (3×100 mL), dried over Na₂SO₄, and concentrated under reduced pressure. The crude product was purified using method RPFC1 (10% to 100% gradient in 20 min) to afford 5-(2-(benzyloxy)-5-fluorophenyl)-4-bromo-1,3-dimethyl-1H-pyrazole (3.2 g, 90%) as a brown solid. m/z: ES⁺ [M+H]⁺=297.25.

### Step 3: 5-(2-(benzyloxy)-5-fluorophenyl)-1,3,4-trimethyl-1H-pyrazole

A mixture of 5-(2-(benzyloxy)-5-fluorophenyl)-4-bromo-1,3-dimethyl-1H-pyrazole (900 mg, 2.4 mmol, 1.0 equiv), methylboronic acid (287 mg, 4.8 mmol, 2.0 equiv), Pd₂(dba)₃ (439 mg, 0.48 mmol, 0.2 equiv), XPhos (229 mg, 0.48 mmol, 0.2 equiv) and tBuONa (692 mg, 7.2 mmol, 3.0 equiv) in dioxane (20 mL) was stirred at 100° C. for 16 h under nitrogen. The resulting mixture then was diluted with water (100 mL) and extracted with EtOAc (3×100 mL), dried over Na₂SO₄, and concentrated under reduced pressure. The crude product was purified using method RPFC1 (10% to 100% gradient in 20 min) to afford 5-(2-(benzyloxy)-5-fluorophenyl)-1,3,4-trimethyl-1H-pyrazole (200 mg, 27%) as a brown oil. m/z: ES⁺ [M+H]⁺=311.10.

### Step 4: 4-fluoro-2-(1,3,4-trimethyl-1H-pyrazol-5-yl)phenol

To a solution of 5-(2-(benzyloxy)-5-fluorophenyl)-1,3,4-trimethyl-1H-pyrazole (200 mg, 0.64 mmol, 1.0 equiv) in 25 mL EtOAc was added Pd/C (10% wt, 150 mg, wet) under nitrogen. The mixture was stirred at room temperature for 16 h under hydrogen, and then filtered through a Celite pad and evaporated to dryness to afford 4-fluoro-2-(1,3,4-trimethyl-1H-pyrazol-5-yl)phenol (100 mg, 70%) as a yellow oil. m/z: ES⁺ [M+H]⁺=221.15.

### 2-(4-cyclopropyl-1,3-dimethyl-1H-pyrazol-5-yl)-4-fluorophenol

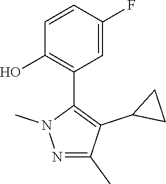 [SMILES: Cc1nn(C)c(-c2cc(F)ccc2O)c1C1CC1]

### Step 1: 5-(2-(benzyloxy)-5-fluorophenyl)-4-cyclopropyl-1,3-dimethyl-1H-pyrazole

A mixture of 5-(2-(benzyloxy)-5-fluorophenyl)-4-bromo-1,3-dimethyl-1H-pyrazole (1.0 g, 2.7 mmol, 1.0 equiv), cyclopropylboronic acid (458 mg, 5.3 mmol, 2.0 equiv), Pd(dppf)Cl₂CH₂Cl₂ (3265 mg, 0.40 mmol, 0.15 equiv), and K₂CO₃ (1.1 g, 8.0 mmol, 3.0 equiv) in dioxane (10 mL) and water (2 mL) was stirred at 100° C. for 16 h under nitrogen. The resulting mixture then was diluted with water (100 mL) and extracted with EtOAc (3×100 mL), dried over Na₂SO₄, and concentrated under reduced pressure. The crude product was purified using method RPFC1 (10% to 100% gradient in 20 min) to afford 5-(2-(benzyloxy)-5-fluorophenyl)-4-cyclopropyl-1,3-dimethyl-1H-pyrazole (200 mg, 27%) as a brown oil. m/z: ES⁺ [M+H]⁺=337.30.

### Step 2: 2-(4-cyclopropyl-1,3-dimethyl-1H-pyrazol-5-yl)-4-fluorophenol

To a solution of 5-(2-(benzyloxy)-5-fluorophenyl)-4-cyclopropyl-1,3-dimethyl-1H-pyrazole (300 mg, 0.89 mmol, 1.0 equiv) in EtOAc (25 mL) was added Pd/C (10% wt, 190 mg, wet) under nitrogen. The mixture was stirred at room temperature for 4 h under hydrogen, and then filtered through a Celite pad and concentrated under reduced pressure to afford 2-(4-cyclopropyl-1,3-dimethyl-1H-pyrazol-5-yl)-4-fluorophenol (100 mg, 46%) as a yellow oil. m/z: ES⁺ [M+H]⁺=247.15.

### 4-fluoro-2-(2-isopropyl-4,5-dimethyl-1H-imidazol-1-yl)phenol

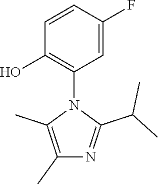 [SMILES: Cc1nc(C(C)C)n(-c2cc(F)ccc2O)c1C]

### Step 1: N-(5-fluoro-2-methoxyphenyl)isobutyrimidamide

To a solution of methyl isobutyrimidate hydrochloride (35 g, 255 mmol, 2.0 equiv) in MeOH (90 mL) was added KOAc (50 g, 511 mmol, 4.0 equiv) at 25° C., and the mixture was stirred for 15 min. A solution of 5-fluoro-2-methoxyaniline (18 g, 128 mmol, 1.0 equiv) in MeOH (90 mL) was then added dropwise, and the mixture was stirred at 25° C. for 5 h. Water (234 mL) was then added and the aqueous mixture was washed with MTBE (2×90 mL). The organic phase was washed with additional water (54 mL), and the combined aqueous layers were pH adjusted to 9.5 with 20% aq. NaOH, and extracted with EtOAc (3×90 mL). The combined organic layers were washed with 10% aq. NaCl (54 mL), and then evaporated to dryness to afford N-(5-fluoro-2-methoxyphenyl)isobutyrimidamide as a purple solid (20 g, 75%).

¹H NMR (400 MHz, methanol-d₄, 300 K) δ 6.94 (dd, J=9.0, 5.1 Hz, 1H), 6.75 (ddd, J=8.9, 8.2, 3.1 Hz, 1H), 6.61 (dd, J=9.5, 3.1 Hz, 1H), 3.78 (s, 3H), 2.67-2.52 (m, 1H), 1.27 (d, J=7.0 Hz, 6H). m/z: ES⁺ [M+H]⁺=211.2.

### Step 2: 1-(5-fluoro-2-methoxyphenyl)-2-isopropyl-4,5-dimethyl-1H-imidazole

To a solution of N-(5-fluoro-2-methoxyphenyl)isobutyrimidamide (20 g, 95 mmol, 1.0 equiv) in iPrOH (100 mL) was added 3-chlorobutan-2-one (23 g, 219 mmol, 2.3 equiv) and Na₂CO₃ (23 g, 219 mmol, 2.3 equiv) at 25° C., and the mixture was heated to reflux for 18 h. Further 3-chlorobutan-2-one (10 g, 95 mmol, 1.0 equiv) and Na₂CO₃ (10 g, 95 mmol, 1.0 equiv) were added and reflux was resumed for an additional 18 h. After colling to 25° C., water (100 mL) was added, pH was adjusted to 1-2 with 4 M aq. HCl, and the mixture was concentrated under vacuum. The resulting mixture was washed with MTBE (100 mL) and iPrOAc (100 mL) was added to the collected aqueous phase. pH was adjusted to 9 with 20% NaOH, and the organic phase was separated and washed with 10% NaCl (100 mL), and then evaporated to dryness to afford 1-(5-fluoro-2-methoxyphenyl)-2-isopropyl-4,5-dimethyl-1H-imidazole as a brown solid (23 g, 87%).

¹H NMR (400 MHz, CDCl₃, 300 K) δ 7.16 (ddd, J=9.1, 7.7, 3.1 Hz, 1H), 7.00 (dd, J=9.1, 4.8 Hz, 1H), 6.91 (dd, J=8.1, 3.1 Hz, 1H), 3.77 (s, 3H), 2.70-2.55 (m, 1H), 2.22 (s, 3H), 1.86 (s, 3H), 1.20 (dd, J=15.0, 6.9 Hz, 6H). m/z: ES⁺ [M+H]⁺=263.2.

### Step 3: 4-fluoro-2-(2-isopropyl-4,5-dimethyl-1H-imidazol-1-yl)phenol

To a solution of 1-(5-fluoro-2-methoxyphenyl)-2-isopropyl-4,5-dimethyl-1H-imidazole (30 g, 114 mmol, 1.0 equiv) in DMAc (300 mL) was added 1-dodecanethiol (104 g, 515 mmol, 4.5 equiv) and NaOH (16 g, 400 mmol, 3.5 equiv) at 25° C., and the mixture was heated to 110° C. for 1 h. After cooling to 25° C., water (150 mL) was added, pH was adjusted to 7 with 4 M aq. HCl, and iPrOAc (150 mL) was added and the mixture was stirred for 12 h. The mixture was then filtered, and the precipitate was washed with iPrOAc/water (1:1, 150 mL), and then slurried in iPrOAc/water (10:3, 390 mL) for 15 h. The solid was then dried under vacuum at 45° C. to afford 4-fluoro-2-(2-isopropyl-4,5-dimethyl-1H-imidazol-1-yl)phenol as a light gray solid (23 g, 80%).

¹H NMR (400 MHz, methanol-d₄, 300 K) δ 7.13 (ddd, J=9.0, 8.0, 3.1 Hz, 1H), 7.03-6.92 (m, 2H), 2.77-2.63 (m, 1H), 2.16 (s, 3H), 1.89 (s, 3H), 1.19 (d, J=7.0 Hz, 6H). m/z: ES⁺ [M+H]⁺=249.0.

### 4-fluoro-2-(5-isopropyl-1,3-dimethyl-1H-pyrazol-4-yl)phenol

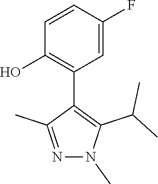 [SMILES: Cc1nn(C)c(C(C)C)c1-c1cc(F)ccc1O]

### Step 1: 4-(2-(benzyloxy)-5-fluorophenyl)-5-isopropyl-1,3-dimethyl-1H-pyrazole

A mixture of 4-bromo-5-isopropyl-1,3-dimethylpyrazole (1.4 g, 6.5 mmol, 1.0 equiv), (2-(benzyloxy)-5-fluorophenyl)boronic acid (3.2 g, 13 mmol, 2.0 equiv), Pd(dppf)Cl₂CH₂Cl₂ (1.1 g, 1.3 mmol, 0.2 equiv) and K₂CO₃ (2.7 g, 19 mmol, 3.0 equiv) in dioxane (20 mL) and water (4 mL) was stirred at 100° C. for 4 h under nitrogen. The resulting mixture was diluted with water (100 mL) and extracted with EtOAc (3×100 mL). The combined organic layers were dried over Na₂SO₄, and concentrated under reduced pressure. The crude product was purified using method RPFC1 (10% to 100% gradient in 20 min) to afford 4-(2-(benzyloxy)-5-fluorophenyl)-5-isopropyl-1,3-dimethyl-1H-pyrazole (1.5 g, 69%) as a brown solid. m/z: ES⁺ [M+H]⁺=339.10.

### Step 2: 4-fluoro-2-(5-isopropyl-1,3-dimethyl-1H-pyrazol-4-yl)phenol

To a solution of 4-(2-(benzyloxy)-5-fluorophenyl)-5-isopropyl-1,3-dimethyl-1H-pyrazole (1.4 g, 4.1 mmol, 1.0 equiv) in MeOH (25 mL) was added Pd/C (10% wt, 600 mg, wet) under nitrogen. The mixture was stirred at room temperature for 16 h under hydrogen, filtered through a Celite pad and concentrated under reduced pressure to afford 4-fluoro-2-(5-isopropyl-1,3-dimethyl-1H-pyrazol-4-yl)phenol (1.0 g, 97%) as a brown solid. m/z: ES⁺ [M+H]⁺=249.20.

### 4-fluoro-2-(3-isopropyl-1,5-dimethyl-1H-pyrazol-4-yl)phenol

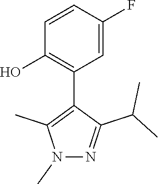 [SMILES: Cc1c(-c2cc(F)ccc2O)c(C(C)C)nn1C]

### Step 1: 4-(2-(benzyloxy)-5-fluorophenyl)-3-isopropyl-1,5-dimethyl-1H-pyrazole

A solution of 4-bromo-3-isopropyl-1,5-dimethyl-1H-pyrazole (500 mg, 2.3 mmol, 1.0 equiv.), (2-(benzyloxy)-5-fluorophenyl)boronic acid (850 mg, 3.5 mmol, 1.5 equiv.), Pd(dppf)Cl₂CH₂Cl₂ (282 mg, 0.35 mmol, 0.15 equiv.) and K₂CO₃ (637 mg, 4.6 mmol, 2.0 equiv.) in dioxane (6 mL) and water (2 mL) was stirred at 100° C. for 16 h under nitrogen. The reaction was quenched with sat. NH₄Cl at room temperature and extracted with EtOAc (3×100 mL), dried over Na₂SO₄, and concentrated under reduced pressure. The residue was purified using method RPFC1 (10% to 100% gradient in 20 min) to afford 4-(2-(benzyloxy)-5-fluorophenyl)-3-isopropyl-1,5-dimethyl-1H-pyrazole (500 mg, 64%) as a brown oil. m/z: ES⁺ [M+H]⁺=339.10.

### Step 2: 4-fluoro-2-(3-isopropyl-1,5-dimethyl-1H-pyrazol-4-yl)phenol

A mixture of 4-(2-(benzyloxy)-5-fluorophenyl)-3-isopropyl-1,5-dimethyl-1H-pyrazole (800 mg, 2.4 mmol, 1.0 equiv.) and Pd/C (500 mg, 10% wt, wet) in EtOAc (10 mL) was stirred at room temperature for 16 h under hydrogen. The resulting mixture was filtered, the filter cake was washed with EtOAc (50 mL). The filtrate was concentrated under reduced pressure to afford 4-fluoro-2-(3-isopropyl-1,5-dimethyl-1H-pyrazol-4-yl)phenol (550 mg, 94%) as a brown solid. m/z: ES⁺ [M+H]⁺=249.05.

### 2-(4-cyclopropyl-1-isopropyl-1H-pyrazol-5-yl)-4-fluorophenol

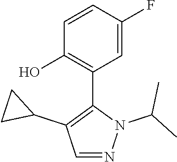 [SMILES: CC(C)n1ncc(C2CC2)c1-c1cc(F)ccc1O]

### Step 1: 5-(2-(benzyloxy)-5-fluorophenyl)-4-cyclopropyl-1-isopropyl-1H-pyrazole

To a stirred mixture of 5-(2-(benzyloxy)-5-fluorophenyl)-4-bromo-1-isopropyl-1H-pyrazole (760 mg, 1.9 mmol, 1.0 equiv), cyclopropylboronic acid (200 mg, 2.3 mmol, 1.2 equiv), K₂CO₃ (810 mg, 5.9 mmol, 3.0 equiv) and PdCl₂(dcypf) (220 mg, 0.29 mmol, 0.15 equiv) in toluene (9 mL) and water (1 mL) were stirred at 100° C. for 16 h under nitrogen. The resulting mixture was diluted with water (60 mL) and extracted with EtOAc (2×60 mL). The combined organic layers were washed with brine (120 mL), dried over Na₂SO₄ and filtered. The filtrate was concentrated under reduced pressure and the residue purified by RPFC1 (40% to 80% gradient in 10 min) to afford 5-(2-(benzyloxy)-5-fluorophenyl)-4-cyclopropyl-1-isopropyl-1H-pyrazole (600 mg, 87%) as a yellow solid. m/z: ES⁺ [M+H]⁺=351.20.

### Step 2: 2-(4-cyclopropyl-1-isopropyl-1H-pyrazol-5-yl)-4-fluorophenol

A solution of 5-(2-(benzyloxy)-5-fluorophenyl)-4-cyclopropyl-1-isopropyl-1H-pyrazole (600 mg, 1.7 mmol, 1.0 equiv) and Pd/C (185 mg, 10% wt, wet) in EtOAc (10 mL) was stirred at room temperature for 16 h under hydrogen. The resulting mixture was filtered and the filter cake washed with EtOAc (2×20 mL). The filtrate was concentrated under reduced pressure to afford 2-(4-cyclopropyl-1-isopropyl-1H-pyrazol-5-yl)-4-fluorophenol (400 mg, 90%) as a light yellow solid. m/z: ES⁺ [M+H]⁺=261.20.

### N-(5-Fluoro-2-hydroxyphenyl)-N-methylisobutyramide

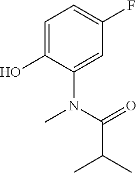

### Step 1: N-(2-(benzyloxy)-5-fluorophenyl)isobutyramide

A solution of 2-(benzyloxy)-5-fluoroaniline (8.9 g, 41 mmol, 1.0 equiv), isobutyryl chloride (4.3 g, 41 mmol, 1.0 equiv) and NEt₃ (1.2 g, 122 mmol, 3.0 equiv) in DCM (50 mL) was stirred at room temperature for 2 h under nitrogen. The residue was then purified using method RPCF4 (10% to 50% gradient in 10 min), yielding N-(2-(benzyloxy)-5-fluorophenyl)isobutyramide (10 g, 73%) as a yellow solid. m/z: ES⁺ [M+H]⁺=288.15.

### Step 2: N-(2-(benzyloxy)-5-fluorophenyl)-N-methylisobutyramide

A solution of N-(2-(benzyloxy)-5-fluorophenyl)isobutyramide (5.0 g, 17 mmol, 1.0 equiv), iodomethane (4.9 g, 35 mmol, 2.0 equiv) and NaH (1.0 g, 44 mmol, 2.5 equiv) in DMF (50 mL) was stirred at room temperature for 2 h under nitrogen. The residue was purified using method RPCF4 (10% to 50% gradient in 10 min), affording N-(2-(benzyloxy)-5-fluorophenyl)-N-methylisobutyramide (4.0 g, 61%) as a yellow oil. m/z: ES⁺ [M+H]⁺=302.10.

### Step 3: N-(5-fluoro-2-hydroxyphenyl)-N-methylisobutyramide

A solution of N-(2-(benzyloxy)-5-fluorophenyl)-N-methylisobutyramide (4.0 g, 13 mmol, 1.0 equiv) and Pd/C (2.8 g, 27 mmol, 2.0 equiv) in MeOH (50 mL) was stirred at room temperature for 2 h under hydrogen. The residue was purified using method RPCF4 (10% to 50% gradient in 10 min), affording N-(5-fluoro-2-hydroxyphenyl)-N-methylisobutyramide (1.8 g, 57%) as a pink solid. m/z: ES⁺ [M+H]⁺=212.05.

### N-ethyl-N-(5-fluoro-2-hydroxyphenyl)isobutyramide

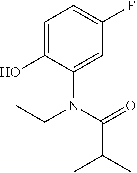 [SMILES: CCN(C(=O)C(C)C)c1cc(F)ccc1O]

### Step 1: N-(2-(benzyloxy)-5-fluorophenyl)-N-ethylisobutyramide

To a stirred solution of N-(2-(benzyloxy)-5-fluorophenyl)isobutyramide (8.5 g, 30 mmol, 1.0 equiv) in DMF (85 mL) was added NaH (2.1 g, 89 mmol, 3.0 equiv) in portions at 0° C. under nitrogen. The resulting mixture was allowed to stir at 0° C. for 20 min under nitrogen, and then iodoethane (9.2 g, 59 mmol, 2.0 equiv) was added dropwise over 5 min at room temperature. The resulting mixture was stirred at room temperature for an additional 1 h, after which the reaction was quenched with water at 0° C. The resulting mixture was extracted with DCM (2×500 mL), the combined organic layers were washed with brine (200 mL), dried over Na₂SO₄. After filtration, the filtrate was concentrated under reduced pressure, and the residue was purified by silica gel column chromatography, eluted with PE/EtOAc (5:1) to afford N-(2-(benzyloxy)-5-fluorophenyl)-N-ethylisobutyramide (8.0 g, 85%) as an off-white solid. m/z: ES⁺ [M+H]⁺=316.15.

### Step 2: N-(5-fluoro-2-hydroxyphenyl)-N-ethylisobutyramide

To a solution of N-(2-(benzyloxy)-5-fluorophenyl)-N-methylisobutyramide (10 g, 32. mmol, 1 equiv) in MeOH (80 mL) was added Pd/C (10%, 5.0 g, 0.15 equiv) and HOAc (100 mL, 1.3 mmol, 0.05 equiv) under nitrogen. The mixture was stirred at room temperature for 2 h under hydrogen, and then filtered through a Celite pad and concentrated under reduced pressure. The residue was purified using method RPCF1 (10% to 100% gradient in 20 min) to afford N-(5-fluoro-2-hydroxyphenyl)-N-ethylisobutyramide (6.6 g, 92%) as an off-white solid. m/z: ES⁺ [M+H]⁺=226.05.

### ((1S,4S)-2-oxa-5-azabicyclo[2.2.1]heptan-5-yl)(5-fluoro-2-hydroxyphenyl)methanone

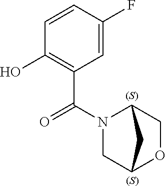

### Step 1: ((1S,4S)-2-oxa-5-azabicyclo[2.2.1]heptan-5-yl)(5-fluoro-2-methoxyphenyl) methanone

The amide coupling between 5-fluoro-2-methoxybenzoic acid (500 mg, 2.9 mmol, 1.0 equiv) and (1S,4S)-2-oxa-5-azabicyclo[2.2.1]heptane hydrochloride (796 mg, 5.9 mmol, 2.0 equiv) was performed according to general procedure A1. The crude product was purified using method RPCF1 (10% to 100% gradient in 20 min) to afford ((1S,4S)-2-oxa-5-azabicyclo[2.2.1]heptan-5-yl)(5-fluoro-2-methoxyphenyl)methanone (500 mg, 68%) as a yellow oil. m/z: ES⁺ [M+H]⁺=252.15.

### Step 2: ((1S,4S)-2-oxa-5-azabicyclo[2.2.1]heptan-5-yl)(5-fluoro-2-hydroxyphenyl) methanone

A mixture of ((1S,4S)-2-oxa-5-azabicyclo[2.2.1]heptan-5-yl)(5-fluoro-2-methoxyphenyl)methanone (490 mg, 2.0 mmol, 1.0 equiv) and BBr₃ (977 mg, 3.9 mmol, 2.0 equiv) in DCM (10 mL) was stirred at −20° C. for 1 h under nitrogen. The reaction was then quenched by the addition of MeOH (100 mL) at 0° C., and the resulting mixture was concentrated under vacuum to afford crude ((1S,4S)-2-oxa-5-azabicyclo[2.2.1]heptan-5-yl)(5-fluoro-2-hydroxyphenyl)methanone (300 mg, quant.) as a yellow oil. m/z: ES⁺ [M+H]⁺=238.15.

### ((1R,4R)-2-oxa-5-azabicyclo[2.2.1]heptan-5-yl)(5-fluoro-2-hydroxyphenyl)methanone

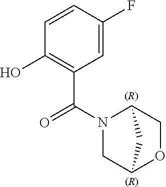

### Step 1: ((1R,4R)-2-oxa-5-azabicyclo[2.2.1]heptan-5-yl)(5-fluoro-2-methoxyphenyl) methanone

The amide coupling between 5-fluoro-2-methoxybenzoic acid (500 mg, 2.9 mmol, 1.0 equiv) and (1R,4R)-2-oxa-5-azabicyclo[2.2.1]heptane hydrochloride (350 mg, 3.5 mmol, 1.2 equiv) was performed according to general procedure A1. The crude product was purified using method RPCF1 (40% to 60% gradient in 20 min) to afford ((1R,4R)-2-oxa-5-azabicyclo[2.2.1]heptan-5-yl) (5-fluoro-2-methoxyphenyl)methanone (400 mg, 54%) as a yellow oil. m/z: ES⁺ [M+H]⁺=252.15.

### Step 2: ((1R,4R)-2-oxa-5-azabicyclo[2.2.1]heptan-5-yl)(5-fluoro-2-hydroxyphenyl) methanone

A mixture of ((1R,4R)-2-oxa-5-azabicyclo[2.2.1]heptan-5-yl)(5-fluoro-2-methoxyphenyl)methanone (250 mg, 1.0 mmol, 1.0 equiv) and BBr₃ (748 mg, 3.0 mmol, 3.0 equiv) in DCM (2 mL) was stirred at −20° C. for 1 h under nitrogen. The reaction was then quenched by the addition of MeOH (100 mL) at 0° C., and the resulting mixture was concentrated under vacuum to afford crude ((1R,4R)-2-oxa-5-azabicyclo[2.2.1]heptan-5-yl)(5-fluoro-2-hydroxyphenyl)methanone (200 mg, 85.) as a yellow oil. m/z: ES⁺ [M+H]⁺=238.05.

### ((3S,5S)-3,5-dimethylmorpholino)(5-fluoro-2-hydroxyphenyl)methanone

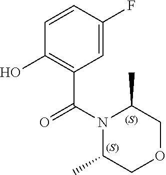

### Step 1: ((3S,5S)-3,5-dimethylmorpholino)(5-fluoro-2-methoxyphenyl)methanone

The amide coupling between 5-fluoro-2-methoxybenzoic acid (500 mg, 2.9 mmol, 1.0 equiv) and (3S,5S)-3,5-dimethylmorpholine (370 g, 1.1 equiv) was performed according to general procedure A1. The residue was purified using method RPCF1 (10% to 50% gradient in 10 min) to afford ((3S,5S)-3,5-dimethylmorpholino)(5-fluoro-2-methoxyphenyl) methanone (720 g, 92%) as a brown solid. m/z: ES⁺ [M+H]⁺=268.10.

### Step 2: ((3S,5S)-3,5-dimethylmorpholino)(5-fluoro-2-hydroxyphenyl)methanone

A solution of ((3S,5S)-3,5-dimethylmorpholino)(5-fluoro-2-methoxyphenyl)methanone (650 mg, 2.4 mmol, 1.0 equiv) and BBr₃ (1.8 g, 7.3 mmol, 3.0 equiv) in DCM (7 mL) was stirred at room temperature for 1 h. The reaction mixture was then quenched with sat. NaHCO₃ at room temperature, and extracted with DCM (2×50 mL). The combined organic layers were washed with brine (2×100 mL), and dried over Na₂SO₄. After filtration, the filtrate was concentrated under reduced pressure to afford ((3S,5S)-3,5-dimethylmorpholino)(5-fluoro-2-hydroxyphenyl)methanone (600 mg, 97%) as a brown solid. m/z: ES⁺ [M+H]⁺=254.10.

### 2-(3-cyclopropylimidazol-4-yl)-4-fluorophenol

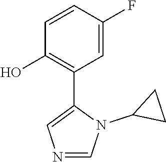 [SMILES: Oc1ccc(F)cc1-c1cncn1C1CC1]

### Step 1: 5-(2-(benzyloxy)-5-fluorophenyl)-1-cyclopropyl-1H-imidazole

A solution of 5-bromo-1-cyclopropylimidazole (400 mg, 2.1 mmol, 1.0 equiv), 2-(benzyloxy)-5-fluorophenylboronic acid (631 mg, 2.6 mmol, 1.2 equiv), Pd(dppf)Cl₂CH₂Cl₂ (349 mg, 0.43 mmol, 0.2 equiv) and K₂CO₃ (887 mg, 6.4 mmol, 3.0 equiv) in 1,4-dioxane/H₂O (6:1, 12 mL) was stirred at 100° C. for 16 h. The resulting mixture was then diluted with water, and extracted with EtOAc. The combined organic layers were washed with water, dried over Na₂SO₄, and concentrated under reduced pressure. The residue was purified using method RPCF4 (30% to 70% gradient in 10 min), resulting in 5-(2-(benzyloxy)-5-fluorophenyl)-1-cyclopropyl-1H-imidazole (180 mg, 27%) as a light yellow oil. m/z: ES⁺ [M+H]⁺=309.05.

### Step 2: 2-(3-cyclopropylimidazol-4-yl)-4-fluorophenol

A solution of 5-(2-(benzyloxy)-5-fluorophenyl)-1-cyclopropyl-1H-imidazole (180 mg, 0.58 mmol, 1.0 equiv) and Pd/C (186 mg, 1.8 mmol, 3 equiv) in MeOH (5 mL) was stirred at room temperature for 3 h under hydrogen. The resulting mixture was filtered and evaporated to dryness to afford 2-(3-cyclopropylimidazol-4-yl)-4-fluorophenol (100 mg, 79%), which was used without further purification. m/z: ES⁺ [M+H]⁺=219.10.

### 2-(2-cyclopropyl-1H-imidazol-1-yl)-4-fluorophenol

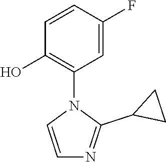 [SMILES: Oc1ccc(F)cc1-n1ccnc1C1CC1]

### Step 1: 2-cyclopropyl-1-(5-fluoro-2-methoxyphenyl)-1H-imidazole

To a stirred mixture of 5-fluoro-2-methoxyaniline (6.0 g, 43 mmol, 1.0 equiv) and cyclopropanecarbaldehyde (3.0 g, 43 mmol, 1.0 equiv) in MeOH (60 mL) were added glyoxal (6.2 g, 43 mmol, 1.0 equiv) and NH₄OAc (3.3 g, 43 mmol, 1.0 equiv) in portions at room temperature under nitrogen. The resulting mixture was stirred at room temperature overnight under nitrogen. The resulting mixture was concentrated under vacuum, and the residue was purified using method RPCF1 (20% to 80% gradient in 30 min). This resulted in 2-cyclopropyl-1-(5-fluoro-2-methoxyphenyl)-1H-imidazole (4.3 g, 44%) as a brown oil. m/z: ES⁺ [M+H]⁺=233.20.

### Step 2: 2-(2-cyclopropyl-1H-imidazol-1-yl)-4-fluorophenol

Deprotection of the phenol was performed using the BBr₃ method described in step #2 of general procedure F1. Evaporation to dryness afforded crude 2-(2-cyclopropyl-1H-imidazol-1-yl)-4-fluorophenol (2.3 g, quant.) as a brown solid. m/z: ES⁺ [M+H]⁺=219.00.

### 2-(5-cyclopropyl-2-methyl-1H-imidazol-1-yl)-4-fluorophenol

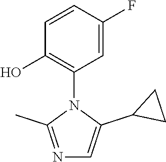 [SMILES: Cc1ncc(C2CC2)n1-c1cc(F)ccc1O]

### Step 1: 1-(2-(benzyloxy)-5-fluorophenyl)-2-methyl-1H-imidazole

A solution of 2-(benzyloxy)-5-fluorophenylboronic acid (10 g, 41 mmol, 1.0 equiv), Cu(OAc)₂ (1.5 g, 8.1 mmol, 0.2 equiv), 2-methylimidazole (5.0 g, 61 mmol, 1.5 equiv), and NEt₃ (8.2 g, 81 mmol, 2 equiv) in MeOH (20 mL) was stirred at room temperature for 1 h. The resulting mixture was diluted with water (200 mL) and extracted with DCM (2×200 mL). The combined organic layers were washed with brine (4×40 mL), dried over Na₂SO₄ and concentrated under reduced pressure. The residue was purified using method RPCF1 (30% to 70% gradient in 10 min), to give 1-(2-(benzyloxy)-5-fluorophenyl)-2-methyl-1H-imidazole (2.5 g, 22%) as a yellow oil. m/z: ES⁺ [M+H]⁺=283.10.

### Step 2: 1-(2-(benzyloxy)-5-fluorophenyl)-5-bromo-2-methyl-1H-imidazole

A solution 1-(2-(benzyloxy)-5-fluorophenyl)-2-methyl-1H-imidazole (2.4 g, 8.5 mmol, 1.0 equiv) and NBS (2.4 g, 8.5 mmol, 1.0 equiv) in DMF (5 mL) was stirred at room temperature for 1 h. The resulting mixture was diluted with water (200 mL) and extracted with DCM (2×200 mL). The combined organic layers were washed with brine (4×40 mL), dried over Na₂SO₄ and concentrated under reduced pressure. The residue was purified using method RPCF1 (30% to 70% gradient in 10 min), to give 1-(2-(benzyloxy)-5-fluorophenyl)-5-bromo-2-methyl-1H-imidazole (1.7 g, 55%) as a yellow oil. m/z: ES⁺ [M+H]⁺=360.95.

### Step 3: 1-(2-(benzyloxy)-5-fluorophenyl)-5-cyclopropyl-2-methyl-1H-imidazole

A solution 1-(2-(benzyloxy)-5-fluorophenyl)-5-bromo-2-methyl-1H-imidazole (1.7 g, 4.6 mmol, 1.0 equiv), cyclopropylboronic acid (589 mg, 6.9 mmol, 1.5 equiv), PdCl₂(dcypf) (518 mg, 0.69 mmol, 0.15 equiv) and Cs₂CO₃ (3.0 g, 9.1 mmol, 2.0 equiv) in toluene (8 mL) was stirred at room temperature for 1 h under nitrogen. The resulting mixture was diluted with water (100 mL) and extracted with DCM (2×100 mL). The combined organic layers were washed with brine (4×40 mL), dried over Na₂SO₄ and concentrated under reduced pressure. The residue was purified using method RPCF1 (30% to 70% gradient in 10 min), to give 1-(2-(benzyloxy)-5-fluorophenyl)-5-cyclopropyl-2-methyl-1H-imidazole (850 mg, 58%) as a yellow oil. m/z: ES⁺ [M+H]⁺=323.10.

### Step 4: 2-(5-cyclopropyl-2-methyl-1H-imidazol-1-yl)-4-fluorophenol

A solution give 1-(2-(benzyloxy)-5-fluorophenyl)-5-cyclopropyl-2-methyl-1H-imidazole (800 mg, 2.5 mmol, 1.0 equiv), and Pd/C (396 mg, 3.7 mmol, 1.5 equiv) in EtOAc was stirred at room temperature for 1 h under hydrogen. The mixture was then filtered through Celite and concentrated under reduced pressure to afford 2-(5-cyclopropyl-2-methyl-1H-imidazol-1-yl)-4-fluorophenol (400 mg, 69%) as a yellow oil. m/z: ES⁺ [M+H]⁺=233.05.

### 4-fluoro-2-(5-methoxy-2-methyl-1H-imidazol-1-yl)-4-fluorophenol

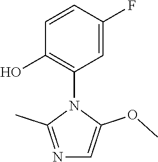

### Step 1: 1-(2-(benzyloxy)-5-fluorophenyl)-5-methoxy-2-methyl-1H-imidazole

To a stirred solution of NaH (100 mg, 4.2 mmol, 1.5 equiv) in MeOH (0.5 mL) were added CuI (105 mg, 0.55 mmol, 0.2 equiv) and a solution of 1-(2-(benzyloxy)-5-fluorophenyl)-5-bromo-2-methyl-1H-imidazole (1.0 g, 2.8 mmol, 1.0 equiv) in DMF (10 mL). The resulting mixture was stirred at 120° C. overnight under nitrogen, and then diluted with water (100 mL) and extracted with DCM (2×100 mL). The combined organic layers were washed with brine (2×200 mL), dried over Na₂SO₄, and concentrated under reduced pressure. The residue was purified using method RPCF1 (20% to 60% gradient in 10 min), to give 1-(2-(benzyloxy)-5-fluorophenyl)-5-methoxy-2-methyl-1H-imidazole (750 mg, yield missing) as a yellow oil. (ES, m/z): [M+H]⁺=313.15.

### Step 2: 4-fluoro-2-(5-methoxy-2-methyl-1H-imidazol-1-yl)-4-fluorophenol

To a solution of 1-(2-(benzyloxy)-5-fluorophenyl)-5-methoxy-2-methyl-1H-imidazole (1.0 g, 3.2 mmol, 1 equiv) in EtOAc (10 mL) was added Pd/C (68 mg, 10% wt) under nitrogen. The mixture was hydrogenated at room temperature for 3 h under hydrogen, and then filtered through Celite and concentrated under reduced pressure. The residue was purified method RPCF1 (20% to 60% gradient in 10 min), to give 4-fluoro-2-(5-methoxy-2-methylimidazol-1-yl) phenol (200 mg, 28%) as alight yellow solid. (ES, m/z): [M+H]⁺=223.10.

### 4-fluoro-2-(5-isopropoxy-2-methyl-1H-imidazol-1-yl)-4-fluorophenol

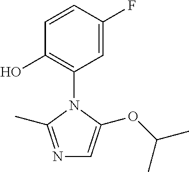 [SMILES: Cc1ncc(OC(C)C)n1-c1cc(F)ccc1O]

### Step 1: 1-(2-(benzyloxy)-5-fluorophenyl)-5-isopropoxy-2-methyl-1H-imidazole

To a stirred solution of NaH (100 mg, 4.2 mmol, 1.5 equiv) in MeOH (0.5 mL) were added CuI (105 mg, 0.55 mmol, 0.2 equiv) and a solution of 1-(2-(benzyloxy)-5-fluorophenyl)-5-bromo-2-methyl-1H-imidazole (1.0 g, 2.8 mmol, 1.0 equiv) in DMF (10 mL). The resulting mixture was stirred at 120° C. overnight under nitrogen, and then diluted with water (100 mL) and extracted with DCM (2×100 mL). The combined organic layers were washed with brine (2×200 mL), dried over Na₂SO₄, and concentrated under reduced pressure. The residue was purified using method RPCF1 (30% to 70% gradient in 10 min), to give 1-(2-(benzyloxy)-5-fluorophenyl)-5-isopropoxy-2-methyl-1H-imidazole (700 mg, 74%) as a yellow oil. (ES, m/z): [M+H]⁺=341.15.

### Step 2: 4-fluoro-2-(5-isopropoxy-2-methyl-1H-imidazol-1-yl)-4-fluorophenol

To a solution of 1-(2-(benzyloxy)-5-fluorophenyl)-5-methoxy-2-methyl-1H-imidazole (700 mg, 2.1 mmol, 1 equiv) in EtOAc (7 mL) was added Pd/C (49 mg, 10% wt) under nitrogen. The mixture was hydrogenated at room temperature for 3 h under hydrogen, and then filtered through Celite and concentrated under reduced pressure. The residue was purified method RPCF1 10% to 50% gradient in 10 min), to give 4-fluoro-2-(5-isopropoxy-2-methylimidazol-1-yl) phenol (200 mg, 28%) as alight yellow solid. (ES, m/z): [M+H]⁺=251.15.

### 2-(5-cyclopropyl-2-isopropylimidazol-1-yl)-4-fluorophenol

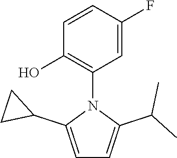 [SMILES: CC(C)c1ccc(C2CC2)n1-c1cc(F)ccc1O]

### Step 1: 1-(5-fluoro-2-methoxyphenyl)-2-isopropylimidazole

A mixture of 5-fluoro-2-methoxyaniline (20 g, 142 mmol, 1.0 equiv) and isobutyraldehyde (10 g, 142 mmol, 1.0 equiv) and glyoxal (8.2 g, 142 mmol, 1.0 equiv) and NH₄OAc (11 g, 142 mmol, 1.0 equiv) in EtOH (200 mL) was stirred at room temperature overnight. The resulting mixture was concentrated under reduced pressure and the residue was purified by silica gel column chromatography, eluting with PE/EtOAc (1:1) to afford 1-(5-fluoro-2-methoxyphenyl)-2-isopropylimidazole (10 g, 30%) as a yellow oil. m/z: ES⁺ [M+H]⁺=235.15.

### Step 2: 5-bromo-1-(5-fluoro-2-methoxyphenyl)-2-isopropylimidazole

A mixture of 1-(5-fluoro-2-methoxyphenyl)-2-isopropylimidazole (10 g, 43 mmol, 1.0 equiv) and NBS (7.6 g, 43 mmol, 1.0 equiv) in MeCN (100 mL) was stirred at room temperature for 2 h. The resulting mixture was concentrated under vacuum and the residue was purified by silica gel column chromatography, eluting with PE/EtOAc (1:1) to afford 5-bromo-1-(5-fluoro-2-methoxyphenyl)-2-isopropylimidazole (9.7 g, 73%) as a yellow oil. m/z: ES⁺ [M+H]⁺=483.20.

### Step 3: 5-cyclopropyl-1-(5-fluoro-2-methoxyphenyl)-2-isopropylimidazole

A mixture of 5-bromo-1-(5-fluoro-2-methoxyphenyl)-2-isopropylimidazole (1.6 g, 5 mmol, 1.0 equiv), cyclopropylboronic acid (1.5 g, 18 mmol, 3.5 equiv), Pd(PPh₃)₄ (0.59 g, 0.5 mmol, 0.1 equiv) and K₃PO₄ (3.8 g, 18 mmol, 3.5 equiv) in dioxane (15 mL) and water (2 mL) was stirred at 80° C. overnight. The resulting mixture was extracted with DCM (3×10 mL), dried over Na₂SO₄, filtered and concentrated to give 5-cyclopropyl-1-(5-fluoro-2-methoxyphenyl)-2-isopropylimidazole (1.0 g, 71%) as a brown oil. m/z: ES⁺ [M+H]⁺=275.15.

### Step 4: 2-(5-cyclopropyl-2-isopropylimidazol-1-yl)-4-fluorophenol

A mixture of 5-cyclopropyl-1-(5-fluoro-2-methoxyphenyl)-2-isopropylimidazole (500 mg, 1.8 mmol, 1.0 equiv) and BBr₃ (4 mL) in DCM (1 mL) was stirred at 50° C. for 3 h. The reaction was quenched with MeOH at 0° C. and concentrated to give a residue that was purified by RFPC1 (10% to 50% gradient in 10 min) to give 2-(5-cyclopropyl-2-isopropylimidazol-1-yl)-4-fluorophenol (250 mg, 53%) as a light yellow oil. m/z: ES⁺ [M+H]⁺=261.15.

### 2-(2,5-dimethyl-1H-imidazol-1-yl)-4-fluorophenol

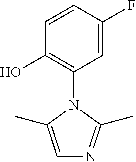

The title phenol was prepared according to general procedure F1.

### Step 1: 1-(5-fluoro-2-methoxyphenyl)-2,5-dimethyl-1H-imidazole

Obtained from the reaction between N-(prop-2-yn-1-yl)acetamide (300 mg, 3.1 mmol, 1.0 equiv) and 5-fluoro-2-methoxyaniline (436 mg, 3.1 mmol, 1.0 equiv). The resulting mixture was concentrated under reduced pressure and purified using method RPCF1 (10% to 50% gradient in 20 min) to afford 1-(5-fluoro-2-methoxyphenyl)-2,5-dimethyl-1H-imidazole (130 mg, 19%) as a yellow oil. m/z: ES⁺ [M+H]⁺=221.05.

### Step 2: 2-(2,5-dimethyl-1H-imidazol-1-yl)-4-fluorophenol

Purified using method RPCF1 (10% to 50% gradient in 10 min) to afford 2-(2,5-dimethyl-1H-imidazol-1-yl)-4-fluorophenol (70 mg, 62%) as a yellow oil. m/z: ES⁺ [M+H]⁺=207.95.

### 2-(2-cyclopropyl-5-methyl-1H-imidazol-1-yl)-4-fluorophenol

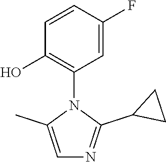 [SMILES: Cc1cnc(C2CC2)n1-c1cc(F)ccc1O]

The title phenol was prepared according to general procedure F1.

### Step 1: 2-cyclopropyl-1-(5-fluoro-2-methoxyphenyl)-5-methyl-1H-imidazole

Obtained from the reaction between N-(prop-2-yn-1-yl)cyclopropanecarboxamide (14 g, 114 mmol, 1.0 equiv) and 5-fluoro-2-methoxyaniline (16.1 g, 114 mmol, 1.0 equiv). The resulting mixture was concentrated under reduced pressure and purified by silica gel column chromatography (PE/EtOAc, 1:2) to afford 2-cyclopropyl-1-(5-fluoro-2-methoxyphenyl)-5-methyl-1H-imidazole (8.0 g, 29%) as a yellow solid. m/z: ES⁺ [M+H]⁺=247.10.

### Step 2: 2-(2-cyclopropyl-5-methyl-1H-imidazol-1-yl)-4-fluorophenol

Purified using method RPCF4 (0% to 100% gradient in 30 min) to afford 2-(2-cyclopropyl-5-methyl-1H-imidazol-1-yl)-4-fluorophenol (6.0 g, 80%) as a yellow solid. m/z: ES⁺ [M+H]⁺=233.15.

### 2-(2-cyclopropyl-5-methyl-1H-imidazol-1-yl)-3,4-difluorophenol

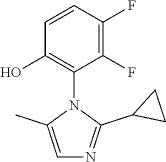 [SMILES: Cc1cnc(C2CC2)n1-c1c(O)ccc(F)c1F]

The title phenol was prepared according to general procedure F1.

### Step 1: 2-cyclopropyl-1-(2,3-difluoro-6-methoxyphenyl)-5-methyl-1H-imidazole

Obtained from the reaction between N-(prop-2-yn-1-yl)cyclopropanecarboxamide (3.5 g, 28 mmol, 1.0 equiv) and 2,3-difluoro-6-methoxyaniline (6.8 g, 43 mmol, 1.5 equiv). The resulting mixture was then concentrated under reduced pressure, and purified using method RPCF5 (0% to 100% gradient in 25 min) to afford 2-cyclopropyl-1-(2,3-difluoro-6-methoxyphenyl)-5-methyl-1H-imidazole (2.5 g, 33%) as a yellow oil. m/z: ES⁺ [M+H]⁺=265.28

### Step 2: 2-(2-cyclopropyl-5-methyl-1H-imidazol-1-yl)-3,4-difluorophenol

Purified using method RPCF5 (0% to 100% gradient in 20 min) to afford 2-(2-cyclopropyl-5-methyl-1H-imidazol-1-yl)-3,4-difluorophenol (2.1 g, 95%) as a yellow oil. m/z: ES⁺ [M+H]⁺=251.19′ 4-fluoro-2-(2-isopropyl-5-methyl-1H-imidazol-1-yl) phenol

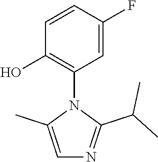 [SMILES: Cc1cnc(C(C)C)n1-c1cc(F)ccc1O]

The title phenol was prepared according to general procedure F1.

### Step 1: 1-(5-Fluoro-2-methoxyphenyl)-2-isopropyl-5-methyl-1H-imidazole

Obtained from the reaction between N-(prop-2-yn-1-yl)isobutyramide (5.0 g, 40 mmol, 1 equiv) and 5-fluoro-2-methoxyaniline (5.64 g, 40 mmol, 1 equiv). The resulting mixture was purified using method RPCF3 (0% B to 100% B in 30 min) to give 1-(5-fluoro-2-methoxyphenyl)-2-isopropyl-5-methyl-1H-imidazole (2.6 g, 26%) as a colourless solid. m/z: ES⁺ [M+H]⁺=249.05.

### Step 2: 4-Fluoro-2-(2-isopropyl-5-methyl-1H-imidazol-1-yl) phenol

Purified using method RPCF4 (0% B to 100% B in 30 min) to give 4-fluoro-2-(2-isopropyl-5-methyl-1H-imidazol-1-yl)phenol (200 mg, 42%) as a colourless solid. m/z: ES⁺ [M+H]⁺=235.05.

### 2-(4,6-dimethylpyrimidin-5-yl)-4-fluorophenol

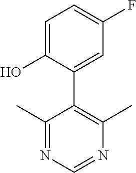 [SMILES: Cc1ncnc(C)c1-c1cc(F)ccc1O]

To a stirred solution of 5-fluoro-2-hydroxyphenylboronic acid (2.0 g, 13 mmol, 1.0 equiv) and 5-bromo-4,6-dimethylpyrimidine (2.9 g, 15 mmol, 1.2 equiv) in 1,4-dioxane (20 mL) and H₂O (2 mL) were added Pd(dppf)Cl₂CH₂Cl₂ (2.1 g, 2.6 mmol, 0.2 equiv) and K₂CO₃ (3.6 g, 26 mmol, 2.0 equiv). The resulting mixture was stirred at 100° C. for 2 h under nitrogen, and then diluted with water (100 mL), and extracted with DCM (2×100 mL). The combined organic layers were washed with water (2×200 mL), dried over Na₂SO₄ and concentrated under reduced pressure. The residue was purified using method RPCF1 (10% to 50% gradient in 10 min) to afford 2-(4,6-dimethylpyrimidin-5-yl)-4-fluorophenol (1.7 g, 61%) as a brown solid. m/z: ES⁺ [M+H]⁺=219.15.

### 2-(4-cyclopropyl-6-methylpyrimidin-5-yl)-4-fluorophenol

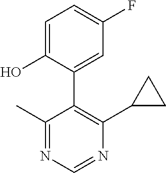 [SMILES: Cc1ncnc(C2CC2)c1-c1cc(F)ccc1O]

To a stirred solution of 5-fluoro-2-hydroxyphenylboronic acid (220 mg, 1.4 mmol, 1.5 equiv) and 5-bromo-4-cyclopropyl-6-methylpyrimidine (200 mg, 0.94 mmol, 1.0 equiv) in 1,4-dioxane (2 mL) and H₂O (0.25 mL) were added Pd(dppf)Cl₂CH₂Cl₂ (35 mg, 0.05 mmol, 0.05 equiv) and K₂CO₃ (259 mg, 1.9 mmol, 2.0 equiv). The resulting mixture was stirred at 100° C. for 16 h under nitrogen, and then diluted with water (10 mL), and extracted with EtOAc (3×20 mL). The combined organic layers were washed with brine (10 mL), dried over Na₂SO₄ and concentrated under reduced pressure. The residue was purified using method RPCF1 (10% to 80% gradient in 10 min) to afford 2-(4-cyclopropyl-6-methylpyrimidin-5-yl)-4-fluorophenol (100 mg, 44%) as a yellow solid. m/z: ES⁺ [M+H]⁺=245.15.

### 2-(4-cyclopropylpyrimidin-5-yl)phenol

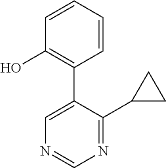 [SMILES: Oc1ccccc1-c1cncnc1C1CC1]

A solution of 5-bromo-4-cyclopropylpyrimidine (800 mg, 4.0 mmol, 1.0 equiv), 2-hydroxyphenylboronic acid (665 mg, 4.8 mmol, 1.2 equiv) and K₂CO₃ (1.1 g, 8.0 mmol, 2.0 equiv) in 1,4-dioxane (5 mL) and water (2.5 mL) was treated with Pd (dppf) Cl₂CH₂Cl₂ (328 mg, 0.4 mmol, 0.1 equiv). The resulting mixture was stirred at 100° C. for 2 h under nitrogen. The resulting mixture was diluted with DCM (200 mL), and the solution was washed with brine (3×50 mL), and was dried over Na₂SO₄. After evaporation, the residue was purified by silica gel column chromatography, eluted with PE:EtOAc (5:1-2:1) to afford 2-(4-cyclopropylpyrimidin-5-yl)phenol (500 mg, 59) as a colourless solid. m/z: ES⁺ [M+H]⁺=213.10.

### 2-(4,6-dicyclopropylpyrimidin-5-yl)-4-fluorophenol

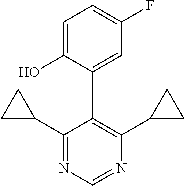 [SMILES: Oc1ccc(F)cc1-c1c(C2CC2)ncnc1C1CC1]

To a stirred solution of 5-fluoro-2-hydroxyphenylboronic acid (74 mg, 47 mmol, 1.5 equiv) and 5-bromo-4,6-dicyclopropylpyrimidine (75 mg, 0.31 mmol, 1.0 equiv) in toluene (1 mL) and H₂O (0.2 mL) were added Pd(dppf)Cl₂CH₂Cl₂ (20 mg, 0.03 mmol, 0.1 equiv) and K₃PO₄ (200 mg, 0.94 mmol, 3.0 equiv). The resulting mixture was stirred at 100° C. for 16 h under nitrogen, and then filtered. The precipitate was washed with EtOAc (3×10 mL) and concentrated under reduced pressure. The residue was purified using method RPCF1 (10% to 80% gradient in 10 min) to afford 2-(4,6-dicyclo propylpyrimidin-5-yl)-4-fluorophenol (30 mg, 35%) as a yellow solid. m/z: ES⁺ [M+H]⁺=271.15.

### 6-(5-fluoro-2-hydroxyphenyl)-1-methylpyridin-2-one

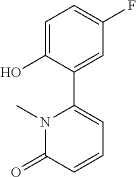 [SMILES: Cn1c(-c2cc(F)ccc2O)cccc1=O]

A solution of 5-fluoro-2-hydroxyphenylboronic acid (200 mg, 1.3 mmol, 1.0 equiv), 6-bromo-1-methylpyridin-2-one (362 mg, 1.9 mmol, 1.5 equiv), Pd(dppf)C₁₂CH₂Cl₂ (210 mg, 0.26 mmol, 0.2 equiv) and K₂CO₃ (532 mg, 3.9 mmol, 3 equiv) in dioxane (5 mL) and H₂O (0.5 mL) was stirred at 100° C. for 2 h under nitrogen. The combined organic layers were washed with brine (4×40 mL), dried over Na₂SO₄. After filtration, the filtrate was concentrated under reduced pressure. The residue was purified using method Prep-HPLC1 (40% to 80% gradient in 10 min) to afford 6-(5-fluoro-2-hydroxyphenyl)-1-methylpyridin-2-one (250 mg, 89%) as a yellow solid. m/z: ES⁺ [M+H]⁺=220.15.

### 2-(2,5-dicyclopropyl-1H-imidazol-1-yl)-4-fluorophenol hydrobromide

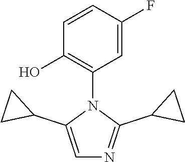 [SMILES: Oc1ccc(F)cc1-n1c(C2CC2)cnc1C1CC1]

### Step 1: 5-bromo-2-cyclopropyl-1-(5-fluoro-2-methoxyphenyl)-1H-imidazole

To a stirred solution of 2-cyclopropyl-1-(5-fluoro-2-methoxyphenyl)-1H-imidazole (1 g, 4.31 mmol, 1 equiv) in MeCN (10 mL) was added NBS (690 mg, 3.9 mmol, 0.9 equiv) in portions at 0° C. under nitrogen. The resulting mixture was stirred at room temperature for 4 h under nitrogen. The resulting mixture was diluted with water (5 mL) then was extracted with EtOAc (3×5 mL). The combined organic layers were washed with brine (3×5 mL), and dried over Na₂SO₄. After filtration, the filtrate was concentrated under reduced pressure, and the residue was purified using method RPCF2 (10% to 100% gradient in 30 min). This resulted in 5-bromo-2-cyclopropyl-1-(5-fluoro-2-methoxyphenyl)-1H-imidazole (680 mg, 51%) as a brown yellow oil. m/z: ES⁺ [M+H]⁺=311.05.

### Step 2: 2,5-dicyclopropyl-1-(5-fluoro-2-methoxyphenyl)-1H-imidazole

To a stirred mixture of 5-bromo-2-cyclopropyl-1-(5-fluoro-2-methoxyphenyl)-1H-imidazole (680 mg, 2.2 mmol, 1.0 equiv) and cyclopropylboronic acid (657 mg, 7.7 mmol, 3.5 equiv) in dioxane/H₂O (7:1, 8 mL) were added Pd(PPh₃)₄ (253 mg, 0.22 mmol, 0.1 equiv) and K₃PO₄ (928 mg, 4.4 mmol, 2.0 equiv) in portions at room temperature under nitrogen. The resulting mixture was stirred at room temperature overnight under nitrogen. The resulting mixture was concentrated under vacuum, and the residue was purified using method RPCF1 (10% to 60% gradient in 25 min) to afford 2,5-dicyclopropyl-1-(5-fluoro-2-methoxyphenyl)-1H-imidazole (300 mg, 50%) as a brown oil. m/z: ES⁺ [M+H]⁺=273.20.

### Step 3: 2-(2,5-dicyclopropyl-1H-imidazol-1-yl)-4-fluorophenol

To a stirred solution of 2,5-dicyclopropyl-1-(5-fluoro-2-methoxyphenyl)imidazole (300 mg, 1.1 mmol, 1.0 equiv) in DCM (3 mL) was added boron tribromide (1 M in DCM, 3 mL) at 0° C. under nitrogen. The resulting mixture was stirred at room temperature overnight under nitrogen. The reaction was quenched with MeOH at 0° C. then the resulting mixture was concentrated under vacuum to afford 2-(2,5-dicyclopropyl-1H-imidazol-1-yl)-4-fluorophenol hydrobromide (250 mg, 88%) as a brown oil. m/z: ES⁺ [M+H]⁺=259.15.

### 2-(2-(tert-butyl)-5-methyl-1H-imidazol-1-yl)-4-fluorophenol

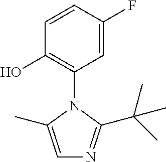 [SMILES: Cc1cnc(C(C)(C)C)n1-c1cc(F)ccc1O]

The title phenol was prepared according to general procedure F1.

### Step 1: 2-(tert-butyl)-1-(5-fluoro-2-methoxyphenyl)-5-methyl-1H-imidazole

Obtained from the reaction between 2,2-dimethyl-N-(prop-2-yn-1-yl)propanamide (2.0 g, 14 mmol, 1 equiv) and 5-fluoro-2-methoxyaniline (2.0 g, 14 mmol, 1 equiv). The resulting mixture was purified by flash chromatography (PE/EtOAc 1:1) to give 2-(tert-butyl)-1-(5-fluoro-2-methoxyphenyl)-5-methyl-1H-imidazole (800 mg, 22%) as a brown solid. m/z: ES⁺ [M+H]⁺=263.15.

### Step 2: 2-(2-(tert-butyl)-5-methyl-1H-imidazol-1-yl)-4-fluorophenol

Purified using method RPCF1 (10% B to 50% B in 10 min) to give 2-(2-(tert-butyl)-5-methyl-1H-imidazol-1-yl)-4-fluorophenol (100 mg, 21%) as a colourless solid. m/z: ES⁺ [M+H]⁺=249.20.

### 6-cyclopropyl-1-(5-fluoro-2-hydroxyphenyl)pyridin-2-one

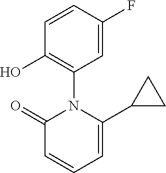 [SMILES: O=c1cccc(C2CC2)n1-c1cc(F)ccc1O]

### Step 1: 6-cyclopropyl-1-(5-fluoro-2-methoxyphenyl)pyridin-2-one

A solution of 2-bromo-4-fluoro-1-methoxybenzene (500 mg, 2.4 mmol, 1.0 equiv), 6-cyclopropyl-1H-pyridin-2-one (350 mg, 2.6 mmol, 1.1 equiv), CuI (150 mg, 0.79 mmol, 0.3 equiv) and K₂CO₃ (1.35 g, 9.8 mmol, 4.0 equiv) in DMF (10 mL) was stirred at 150° C. for 16 h. The resulting mixture was diluted with water (50 mL), extracted with EtOAc (3×20 mL) and the combined organic layers were washed with brine (10 mL), dried over Na₂SO₄, filtered and concentrated under reduced pressure to give a residue that was purified using method RFPC1 (10% to 50% gradient in 10 min) to give 6-cyclopropyl-1-(5-fluoro-2-methoxyphenyl)pyridin-2-one (460 mg, 73%) as a yellow solid. m/z: ES⁺ [M+H]⁺=260.10.

### Step 2: 6-cyclopropyl-1-(5-fluoro-2-hydroxyphenyl)pyridin-2-one

A solution of 6-cyclopropyl-1-(5-fluoro-2-methoxyphenyl)pyridin-2-one (480 mg, 1.9 mmol, 1.0 equiv) and BBr₃ (1.4 g, 5.6 mmol, 3.0 equiv) in DCM (2 mL) was stirred at room temperature for 2 h under nitrogen. The reaction was quenched with MeOH at 0° C. and the residue was purified using method RFPC4 (10% to 50% gradient in 10 min) to afford 6-cyclopropyl-1-(5-fluoro-2-hydroxyphenyl)pyridin-2-one (260 mg, 57%) as a yellow oil. m/z: ES⁺ [M+H]⁺=246.09.

### 4-fluoro-2-(5-methyl-2-(trifluoromethyl)-1H-imidazol-1-yl)phenol

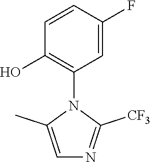 [SMILES: Cc1cnc(C(F)(F)F)n1-c1cc(F)ccc1O]

The title phenol was prepared according to general procedure F1.

### Step 1: 1-(5-fluoro-2-methoxyphenyl)-5-methyl-2-(trifluoromethyl)-1H-imidazole

Obtained from the reaction between 2,2,2-trifluoro-N-(prop-2-yn-1-yl)acetamide (1.2 g, 7.9 mmol, 1.0 equiv) and 5-fluoro-2-methoxyaniline (0.78 g, 5.6 mmol, 1.0 equiv). The resulting mixture was purified using method RFPC4 (40% to 50% gradient in 10 min) to give 1-(5-fluoro-2-methoxyphenyl)-5-methyl-2-(trifluoromethyl)-1H-imidazole (530 mg, 24%) as a light yellow oil. m/z: ES⁺ [M+H]⁺=275.28.

### Step 2: 4-fluoro-2-(5-methyl-2-(trifluoromethyl)-1H-imidazol-1-yl)phenol

Purified using method RPCF3 (70% B to 80% B in 10 min) to give 4-fluoro-2-(5-methyl-2-(trifluoromethyl)-1H-imidazol-1-yl)phenol (200 mg, 42%) as a light yellow solid. m/z: ES⁺ [M+H]⁺=261.33.

### 2-(2-cyclopropyl-5-methoxy-1H-imidazol-1-yl)-4-fluorophenol

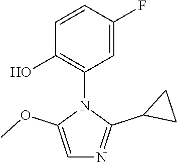 [SMILES: COc1cnc(C2CC2)n1-c1cc(F)ccc1O]

### Step 1: 1-(2-(benzyloxy)-5-fluorophenyl)-2-cyclopropyl-1H-imidazole

To a stirred mixture of (2-(benzyloxy)-5-fluorophenyl)boronic acid (5.0 g, 20 mmol, 1.0 equiv) and 2-cyclopropyl-1H-imidazole (3.3 g, 30 mmol, 1.5 equiv) in MeOH (50 mL) were added Cu(OAc)₂ (1.9 g, 10 mmol, 1.0 equiv) and NEt₃ (4.1 g, 40 mmol, 2.0 equiv) at room temperature under nitrogen. The resulting mixture was stirred at room temperature overnight under nitrogen. The resulting mixture was quenched by the addition of water (50 mL) at 0° C. and extracted with EtOAc (3×50 mL). The combined organic layers were washed with brine (2×50 mL), and dried over Na₂SO₄. After filtration, the filtrate was concentrated under reduced pressure, and the residue was purified using method RPCF2 (10% to 100% gradient in 20 min). This resulted in 1-(2-(benzyloxy)-5-fluorophenyl)-2-cyclopropyl-1H-imidazole (1.6 g, 26%) as a brown oil. m/z: ES⁺ [M+H]⁺=309.10.

### Step 2: 1-(2-(benzyloxy)-5-fluorophenyl)-5-bromo-2-cyclopropyl-1H-imidazole

A mixture of 1-(2-(benzyloxy)-5-fluorophenyl)-2-cyclopropyl-1H-imidazole (600 mg, 1.9 mmol, 1.0 equiv) and NBS (312 mg, 1.75 mmol, 0.9 equiv) in MeCN (6 mL) was stirred at room temperature for 3 h under nitrogen. The resulting mixture was quenched by the addition of water (20 mL) at 0° C., and extracted with EtOAc (3×20 mL). The combined organic layers were washed with brine (2×30 mL), dried over Na₂SO₄, and concentrated under reduced pressure. The residue was purified using method RPCF1 (10% to 100% gradient in 25 min). This resulted in 1-(2-(benzyloxy)-5-fluorophenyl)-5-bromo-2-cyclopropyl-1H-imidazole (320 mg, 42%) as a brown oil. m/z: ES⁺ [M+H]⁺=387.05.

### Step 3: 1-(2-(benzyloxy)-5-fluorophenyl)-2-cyclopropyl-5-methoxy-1H-imidazole

A mixture of 1-(2-(benzyloxy)-5-fluorophenyl)-5-bromo-2-cyclopropyl-1H-imidazole (320 mg, 0.83 mmol, 1.0 equiv) and CuI (32 mg, 0.17 mmol, 0.2 equiv) in MeONa (30% in MeOH, 3 mL) was stirred at 100° C. overnight under nitrogen. The reaction was quenched by the addition of sat. NH₄Cl (20 mL) at 0° C., and extracted with EtOAc (3×20 mL). The combined organic layers were washed with brine (2×25 mL), dried over Na₂SO₄, and concentrated under reduced pressure. The residue was purified RPCF2 (10% to 60% gradient in 20 min). This resulted in 1-(2-(benzyloxy)-5-fluorophenyl)-2-cyclopropyl-5-methoxy-1H-imidazole (100 mg, 36%) as a yellow oil. m/z: ES⁺ [M+H]⁺=339.00.

### Step 4: 2-(2-cyclopropyl-5-methoxy-1H-imidazol-1-yl)-4-fluorophenol

A mixture of 1-(2-(benzyloxy)-5-fluorophenyl)-2-cyclopropyl-5-methoxy-1H-imidazole (100 mg, 0.30 mmol, 1.0 equiv) and Pd/C (25 mg, 10% wt) in EtOAc (1 mL) was stirred at room temperature for 1 h under hydrogen. The mixture was then filtered, the solids washed with MeOH (3×5 mL), and the filtrate concentrated under reduced pressure, to afford in 2-(2-cyclopropyl-5-methoxy-1H-imidazol-1-yl)-4-fluorophenol (60 mg, 82%) as a yellow oil. m/z: ES⁺ [M+H]⁺=249.10.

### 4-fluoro-2-(2,4,5-trimethyl-1H-imidazol-1-yl)phenol

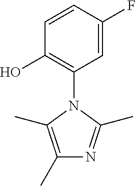

### Step 1: 1-(5-fluoro-2-methoxyphenyl)-2,4,5-trimethyl-1H-imidazole

To a stirred solution of 5-fluoro-2-methoxyaniline (1.0 g, 7.1 mmol, 1.0 equiv) and acetaldehyde (0.31 g, 7.1 mmol, 1.0 equiv) in MeOH (10 mL) were added with acetamide (0.42 g, 7.1 mmol, 1.0 equiv) and diacetyl (0.61 g, 7.1 mmol, 1.0 equiv) at 0° C. under nitrogen. The resulting mixture was quenched by the addition of water (20 mL) at 0° C. and extracted with EtOAc (3×20 mL). The combined organic layers were washed with brine (3×20 mL), dried over anhydrous Na₂SO₄, and concentrated under reduced pressure. The residue was purified using method RPCF1 (40% to 50% gradient in 10 min), resulting in 1-(5-fluoro-2-methoxyphenyl)-2,4,5-trimethyl-1H-imidazole (260 mg, 16%) as a colourless solid. m/z: ES⁺ [M+H]⁺=253.05.

### Step 2: 4-fluoro-2-(2,4,5-trimethyl-1H-imidazol-1-yl)phenol

Deprotection of the phenol was performed using the BBr₃ method described in step #2 of general procedure F1. Purification using method RPCF1 (50% to 60% gradient in 15 min) afforded 4-fluoro-2-(2,4,5-trimethyl-1H-imidazol-1-yl)phenol (150 mg, 66%.) as a colourless solid. m/z: ES⁺ [M+H]⁺=221.05.

### 1-(5-fluoro-2-hydroxyphenyl)-6-isopropylpyridin-2(1H)-one

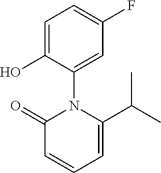 [SMILES: CC(C)c1cccc(=O)n1-c1cc(F)ccc1O]

### Step 1: 1-(5-fluoro-2-methoxyphenyl)-6-isopropylpyridin-2(1H)-one

A solution of 6-isopropylpyridin-2-ol (800 mg, 5.8 mmol, 1.0 equiv), 2-bromo-4-fluoro-1-methoxybenzene (1.2 g, 5.8 mmol, 1.0 equiv), CuI (111 mg, 0.58 mmol, 0.1 equiv) and K₂CO₃ (2.4 mg, 17 mmol, 3.0 equiv) in DMF (8 mL) was stirred at 150° C. for 16 h. The resulting mixture was diluted with water (50 mL) and extracted with EtOAc (2×50 mL). The combined organic layers were washed with water (2×50 mL), dried over Na₂SO₄, and concentrated under reduced pressure. The residue was purified using method RPFC1 (60% to 70% gradient in 10 min), to afford 1-(5-fluoro-2-methoxyphenyl)-6-isopropylpyridin-2-one (150 mg, 10%) as a colourless solid. m/z: ES⁺ [M+H]⁺=262.15.

### Step 2: 1-(5-fluoro-2-hydroxyphenyl)-6-isopropylpyridin-2(1H)-one

A solution of 1-(5-fluoro-2-methoxyphenyl)-6-isopropylpyridin-2-one (150 mg, 0.57 mmol, 1.0 equiv) and BBr₃ (287 mg, 1.1 mmol, 2.0 equiv) in DCM (2 mL) was stirred at room temperature for 4 h. The reaction was quenched by the addition of MeOH (5 mL) and concentrated under reduced pressure to afford 1-(5-fluoro-2-hydroxyphenyl)-6-isopropylpyridin-2(1H)-one (95 mg, 67%) as a yellow solid. m/z: ES⁺ [M+H]⁺=248.05.

### 6-(5-fluoro-2-hydroxyphenyl)-5-isopropyl-1-methylpyridin-2(1H)-one

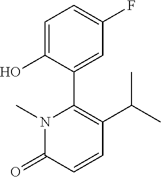 [SMILES: CC(C)c1ccc(=O)n(C)c1-c1cc(F)ccc1O]

### Step 1: 6-chloro-1-methyl-5-(prop-1-en-2-yl)pyridin-2(1H)-one

A mixture of 5-bromo-6-chloro-1-methylpyridin-2(1H)-one (3.0 g, 13 mmol, 1.0 equiv), 4,4,5,5-tetramethyl-2-(prop-1-en-2-yl)-1,3,2-dioxaborolane (1.8 g, 11 mmol, 0.8 equiv), Pd(OAc)₂ (303 mg, 1.3 mmol, 0.1 equiv), tricyclohexylphosphane (756 mg, 2.7 mmol, 0.2 equiv) and K₃PO₄ (5.7 g, 27 mmol, 2.0 equiv) in toluene (50 mL)/H₂O (5 mL) was stirred at 100° C. for 2 h under nitrogen. The mixture was diluted with water and extracted with DCM (2×200 mL). The combined organic layers were washed with brine (2×200 mL), dried over anhydrous Na₂SO₄ and concentrated under reduced pressure. The residue was purified using method RPFC1 (10% to 80% gradient in 20 min), to afford 6-chloro-1-methyl-5-(prop-1-en-2-yl)pyridin-2(1H)-one (1.5 mg, 57%) as a yellow solid. m/z: ES⁺ [M+H]⁺=184.05.

### Step 2: 6-(2-(benzyloxy)-5-fluorophenyl)-1-methyl-5-(prop-1-en-2-yl)pyridin-2(1H)-one

A solution of 6-chloro-1-methyl-5-(prop-1-en-2-yl)pyridin-2(1H)-one (1.5 g, 8.2 mmol, 1.0 equiv), 2-(benzyloxy)-5-fluorophenylboronic acid (2.0 g, 8.2 mmol, 1.0 equiv), RuPhos Pd G3 (683 mg, 0.8 mmol, 0.1 equiv), RuPhos (762 mg, 1.6 mmol, 0.2 equiv) and K₃PO₄ (5.2 g, 25 mmol, 3.0 equiv) in toluene (30 mL)/H₂O (5 mL) was stirred at 100° C. for 4 h under nitrogen. The mixture was diluted with water and extracted with DCM (2×50 mL). The combined organic layers were washed with brine (2×50 mL), dried over anhydrous Na₂SO₄ and concentrated under reduced pressure. The residue was purified using method RPFC1 (10% to 80% gradient in 20 min), to afford 6-(2-(benzyloxy)-5-fluorophenyl)-1-methyl-5-(prop-1-en-2-yl)pyridin-2(1H)-one (600 mg, 21%) as a yellow solid. m/z: ES⁺ [M+H]⁺=350.15.

### Step 3: 6-(5-fluoro-2-hydroxyphenyl)-5-isopropyl-1-methylpyridin-2(1H)-one

A solution of 6-(2-(benzyloxy)-5-fluorophenyl)-1-methyl-5-(prop-1-en-2-yl)pyridin-2(1H)-one (580 mg, 1.7 mmol, 1.0 equiv) and Pd/C (265 mg, 10% wt, wet) in EtOAc (5 mL) was stirred at room temperature for 2 h under hydrogen. The resulting mixture was then filtered, and the filter cake was washed with EtOAc (5×30 mL). The filtrate was then concentrated under reduced pressure to afford 6-(5-fluoro-2-hydroxyphenyl)-5-isopropyl-1-methylpyridin-2(1H)-one (380 mg, 88%) as a yellow solid. m/z: ES⁺ [M+H]⁺=262.12.

### 2-(2-cyclopropyl-4,5-dimethyl-1H-imidazol-1-yl)-4-fluorophenol

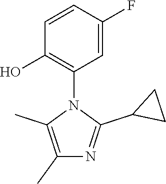 [SMILES: Cc1nc(C2CC2)n(-c2cc(F)ccc2O)c1C]

### Step 1: 2-cyclopropyl-1-(5-fluoro-2-methoxyphenyl)-4,5-dimethyl-1H-imidazole

A solution of 5-fluoro-2-methoxyaniline (1.0 g, 7.1 mmol, 1.0 equiv), biacetyl (610 mg, 7.1 mmol, 1.0 equiv), cyclopropanecarbaldehyde (497 mg, 7.1 mmol, 1.0 equiv), 5 M aq. AcONH4 (1.7 g, 18 mmol, 2.5 equiv) in MeOH (8 mL) was stirred at room temperature for 16 h. The reaction was poured into sat. NH₄Cl (aq.) and extracted with EtOAc (3×50 mL). The combined organic layers were washed with brine (3×30 mL), dried over anhydrous Na₂SO₄, and concentrated under reduced pressure. The resulting residue was purified using method RPFC1 (0% to 100% gradient in 30 min), resulting in 2-cyclopropyl-1-(5-fluoro-2-methoxyphenyl)-4,5-dimethyl-1H-imidazole (280 mg, 9%) as a yellow oil. m/z: ES⁺ [M+H]⁺=261.15.

### Step 2: 2-(2-cyclopropyl-4,5-dimethyl-1H-imidazol-1-yl)-4-fluorophenol

A solution of 2-cyclopropyl-1-(5-fluoro-2-methoxyphenyl)-4,5-dimethyl-1H-imidazole (160 mg, 0.62 mmol, 1.0 equiv) and BBr₃ (616 mg, 2.5 mmol, 4.0 equiv) in DCM (5 mL) was stirred at room temperature for 2 h. The reaction was quenched by the addition sat. NH₄Cl, and with EtOAc (3×10 mL). The combined organic layers were dried over anhydrous Na₂SO₄, and concentrated under reduced pressure to afford 2-(2-cyclopropyl-4,5-dimethyl-1H-imidazol-1-yl)-4-fluorophenol (170 mg, quant.), which was used without further purification. m/z: ES⁺ [M+H]⁺=247.15.

### 4-fluoro-2-(4-isopropyl-1-methyl-1H-1,2,3-triazol-5-yl)phenol

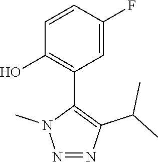 [SMILES: CC(C)c1nnn(C)c1-c1cc(F)ccc1O]

### Step 1: 1-(benzyloxy)-4-fluoro-2-(3-methylbut-1-yn-1-yl)benzene

A solution of 3-methylbut-1-yne (2.0 g, 29 mmol, 1.0 equiv) and 1-(benzyloxy)-2-bromo-4-fluorobenzene (4.0 g, 14 mmol, 0.5 equiv), Pd(PPh₃)₄ (3.4 g, 2.9 mmol, 0.1 equiv), CuI (620 mg, 3.3 mmol, 0.11 equiv), NEt₃ (9.0 g, 89 mmol, 3.0 equiv) in THF (30 mL) was stirred at room temperature for 16 h under nitrogen. The resulting mixture was diluted with water (10 mL) and extracted with EtOAc (3×20 mL). The combined organic layers were washed with brine (10 mL), dried over anhydrous Na₂SO₄, and concentrated under reduced pressure. The residue was purified using method RPFC1 (10% to 50% gradient in 10 min), resulting inl-(benzyloxy)-4-fluoro-2-(3-methylbut-1-yn-1-yl)benzene (2.0 g, 25%) as a yellow oil.

### Step 2: 5-(2-(benzyloxy)-5-fluorophenyl)-4-isopropyl-1-((trimethylsilyl)methyl)-1H-1,2,3-triazole

A solution of 1-(benzyloxy)-4-fluoro-2-(3-methylbut-1-yn-1-yl)benzene (1.0 g, 3.7 mmol, 1.0 equiv), (azidomethyl)trimethylsilane (0.48 g, 3.7 mmol, 1.0 equiv) and bis((1Z,5Z)-cycloocta-1,5-diene); bis(chloroiridium) (0.50 g, 0.75 mmol, 0.2 equiv) in DCM (10 mL) was stirred at room temperature for 16 h under air. The resulting mixture was diluted with water (30 mL) and extracted with DCM (3×20 mL). The combined organic layers were washed with brine (10 mL), dried over anhydrous Na₂SO₄, and concentrated under reduced pressure. The residue was purified by silica gel column chromatography, eluting with PE/EtOAc (2:1) to afford 5-(2-(benzyloxy)-5-fluorophenyl)-4-isopropyl-1-((trimethylsilyl)methyl)-1H-1,2,3-triazole (1.0 g, 67%) as a light-yellow oil. m/z: ES⁺ [M+H]⁺=398.20.

### Step 3: 5-(2-(benzyloxy)-5-fluorophenyl)-4-isopropyl-1-methyl-1H-1,2,3-triazole

A 1 M solution of TBAF in THF (1.3 g, 5.0 mmol, 2.0 equiv) was added dropwise at 0° C. under nitrogen to a stirred solution of 5-(2-(benzyloxy)-5-fluorophenyl)-4-isopropyl-1-((trimethylsilyl)methyl)-1H-1,2,3-triazole (1 g, 2.515 mmol, 1 equiv) in THF (10 mL). The resulting mixture was stirred at room temperature for 3 h under nitrogen, and then diluted with water (30 mL), and extracted with EtOAc (3×30 mL). The combined organic layers were washed with brine (100 mL), dried over anhydrous Na₂SO₄, and concentrated under reduced pressure. The residue was purifed by silica gel column chromatography, eluting with PE/EtOAc (1:1) to afford 5-(2-(benzyloxy)-5-fluorophenyl)-4-isopropyl-1-methyl-1H-1,2,3-triazole (700 mg, 86%) as a light-yellow oil. m/z: ES⁺ [M+H]⁺=326.10.

### Step 4: 4-fluoro-2-(4-isopropyl-1-methyl-1H-1,2,3-triazol-5-yl)phenol

A suspension of 5-(2-(benzyloxy)-5-fluorophenyl)-4-isopropyl-1-methyl-1H-1,2,3-triazole (600 mg, 1.8 mmol, 1.0 equiv) and Pd/C (20 mg, 10% wt, wet) in MeOH (6 mL) was stirred at room temperature for 2 h under hydrogen, and then filtered. The filter cake was washed with MeOH (3×10 mL), and the filtrate was concentrated under reduced pressure. This resulted in 4-fluoro-2-(4-isopropyl-1-methyl-1H-1,2,3-triazol-5-yl)phenol (400 mg, 92%) as a light-yellow oil. m/z: ES⁺ [M+H]⁺=236.10.

### 2-(1,4-diisopropyl-1H-imidazol-5-yl)-4-fluorophenol

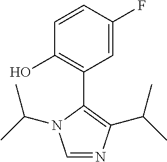 [SMILES: CC(C)c1ncn(C(C)C)c1-c1cc(F)ccc1O]

### Step 1: 5-bromo-1,4-diisopropyl-1H-imidazole

A mixture of 1,4-diisopropyl-1H-imidazole (CAS 15485˜48:5) 750 mg, 4.9 mmol, 1.0 equiv) and NBS (438 mg, 2.5 mmol, 0.5 equiv) in DMF (5 mL) was stirred at room temperature for 1 h under nitrogen. The resulting mixture was then diluted with water (50 mL) and extracted with EtOAc (2×50 mL). The combined organic layers were washed with water (2×50 mL), dried over anhydrous Na₂SO₄, and concentrated under reduced pressure. The crude product was purified using method RPFC1 (10% to 100% gradient in 20 min), resulting in 5-bromo-1,4-diisopropyl-1H-imidazole (320 mg, 28%) as a yellow oil. m/z: ES⁺ [M+H]⁺=231.05.

### Step 2: 5-(2-(benzyloxy)-5-fluorophenyl)-1,4-diisopropyl-1H-imidazole

A mixture of 5-bromo-1,4-diisopropyl-1H-imidazole (300 mg, 1.3 mmol, 1.0 equiv), (2-(benzyloxy)-5-fluorophenyl)boronic acid (638 mg, 2.6 mmol, 2.0 equiv), Pd(dppf)Cl₂CH₂Cl₂ (211 mg, 0.26 mmol, 0.2 equiv) and K₂CO₃ (538 mg, 3.9 mmol, 3.0 equiv) in dioxane/H₂O (5:1, 6 mL) was stirred at 100° C. for 16 h under nitrogen. The resulting mixture was diluted with water (50 mL) and extracted with EtOAc (2×50 mL). The combined organic layers were washed with water (2×50 mL), dried over anhydrous Na₂SO₄, and concentrated under reduced pressure. The crude product was purified using method RPFC1 (10% to 100% gradient in 20 min), resulting in 5-(2-(benzyloxy)-5-fluorophenyl)-1,4-diisopropyl-1H-imidazole (220 mg, 48%) as a brown oil. m/z: ES⁺ [M+H]⁺=353.15.

### Step 3: 2-(1,4-diisopropyl-1H-imidazol-5-yl)-4-fluorophenol

A suspension of 5-(2-(benzyloxy)-5-fluorophenyl)-1,4-diisopropyl-1H-imidazole (200 mg, 0.57 mmol, 1.0 equiv) and Pd/C (10% wt, wet, 319 mg) in MeOH (10 mL) was hydrogenated at room temperature for 2 h under hydrogen. The resulting mixture was then filtered through Celite and concentrated under reduced pressure to afford 2-(1,4-diisopropyl-1H-imidazol-5-yl)-4-fluorophenol (140 mg, 94%) as a light-yellow oil. m/z: ES⁺ [M+H]⁺=263.25.

### 2-(2-ethyl-5-isopropyl-1H-imidazol-1-yl)-4-fluorophenol

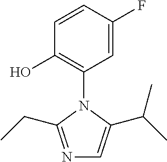 [SMILES: CCc1ncc(C(C)C)n1-c1cc(F)ccc1O]

### Step 1: 2-ethyl-1-(5-fluoro-2-methoxyphenyl)-1H-imidazole

A mixture of 5-fluoro-2-methoxyaniline (10 g, 71 mmol, 1.0 equiv) and propionaldehyde (4.1 g, 71 mmol, 1.0 equiv), oxalaldehyde (10.3 g, 177 mmol, 2.5 equiv) and NH₄OAc (5.5 g, 71 mmol, 1.0 equiv) in EtOH (200 mL) was stirred at room temperature for 16 h. The resulting mixture was diluted with water (100 mL), and extracted with EtOAc (3×100 mL). The combined organic layers were dried over anhydrous Na₂SO₄, and concentrated under reduced pressure. The residue was purified using method RPFC1 (10% to 100% gradient in 40 min), resulting in 2-ethyl-1-(5-fluoro-2-methoxyphenyl)-1H-imidazole (6.5 g, 42%) as a brown solid. m/z: ES⁺ [M+H]⁺=221.25.

### Step 2: 5-bromo-2-ethyl-1-(5-fluoro-2-methoxyphenyl)-1H-imidazole

A mixture of 2-ethyl-1-(5-fluoro-2-methoxyphenyl)-1H-imidazole (3.0 g, 14 mmol, 1.0 equiv) and NBS (2.4 g, 14 mmol, 1.0 equiv) in DMF (15 mL) was stirred at room temperature for 3 h. The resulting mixture was diluted with water (100 mL) and extracted with EtOAc (3×100 mL). The combined organic layers were dried over anhydrous Na₂SO₄ and concentrated under reduced pressure. The residue was purified using method RPFC1 (10% to 100% gradient in 20 min), resulting in 5-bromo-2-ethyl-1-(5-fluoro-2-methoxyphenyl)-1H-imidazole (2.6 g, 64%) as a brown solid. m/z: ES⁺ [M+H]⁺=299.25.

### Step 3: 2-ethyl-1-(5-fluoro-2-methoxyphenyl)-5-(prop-1-en-2-yl)-1H-imidazole

A mixture of 5-bromo-2-ethyl-1-(5-fluoro-2-methoxyphenyl)-1H-imidazole (1.0 g, 3.3 mmol, 1.0 equiv), 4,4,5,5-tetramethyl-2-(prop-1-en-2-yl)-1,3,2-dioxaborolane (1.1 g, 6.7 mmol, 2.0 equiv), Pd(dppf)Cl₂CH₂Cl₂ (0.41 g, 0.5 mmol, 0.15 equiv) and K₃PO₄ (1.4 g, 10 mmol, 3.0 equiv) in dioxane (10 mL) and water (2 mL) was stirred at 100° C. for 16 h under nitrogen. The resulting mixture was diluted with water (100 mL) and extracted with EtOAc (3×100 mL). The combined organic layers were dried over anhydrous Na₂SO₄ and concentrated under reduced pressure. The residue was purified using method RPFC1 (10% to 100% gradient in 20 min), resulting in 2-ethyl-1-(5-fluoro-2-methoxyphenyl)-5-(prop-1-en-2-yl)-1H-imidazole (300 mg, 35%) as a light-yellow oil. m/z: ES⁺ [M+H]⁺=261.30.

### Step 4: 2-ethyl-1-(5-fluoro-2-methoxyphenyl)-5-isopropyl-1H-imidazole

A suspension of 2-ethyl-1-(5-fluoro-2-methoxyphenyl)-5-(prop-1-en-2-yl)-1H-imidazole (300 mg, 1.2 mmol, 1.0 equiv) and Pd/C (10% wt, wet, 125 mg) in MeOH (20 mL) was stirred at room temperature for 1 h under hydrogen, and then filtered through Celite and concentrated under reduced pressure to give 2-ethyl-1-(5-fluoro-2-methoxyphenyl)-5-isopropyl-1H-imidazole (250 mg, 83%) as a light-yellow oil. m/z: ES⁺ [M+H]⁺=263.20.

### Step 5: 2-(2-ethyl-5-isopropyl-1H-imidazol-1-yl)-4-fluorophenol

A mixture of 2-ethyl-1-(5-fluoro-2-methoxyphenyl)-5-isopropyl-1H-imidazo (250 mg, 0.95 mmol, 1.0 equiv) and BBr₃ (716 mg, 2.9 mmol, 3.0 equiv) in DCM (2 mL) was stirred at room temperature for 3 h. The reaction was quenched with MeOH at 0° C. and concentrated under reduced pressure to give 2-(2-ethyl-5-isopropyl-1H-imidazol-1-yl)-4-fluorophenol (190 mg, 80%) as a light-yellow oil. m/z: ES⁺ [M+H]⁺=249.30.

### 4-fluoro-2-(4-isopropyl-1,3-dimethyl-1H-pyrazol-5-yl)phenol

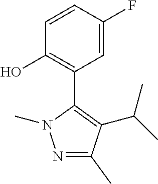 [SMILES: Cc1nn(C)c(-c2cc(F)ccc2O)c1C(C)C]

### Step 1: 5-(2-(benzyloxy)-5-fluorophenyl)-1,3-dimethyl-1H-pyrazole

A solution of 5-bromo-1,3-dimethyl-1H-pyrazole (7.1 g, 41 mmol, 2.0 equiv), (2-(benzyloxy)-5-fluorophenyl)boronic acid (5.0 g, 20 mmol, 1.0 equiv), Pd(dppf)Cl₂CH₂Cl₂ (3.3 g, 4.1 mmol, 0.1 equiv), and K₂CO₃ (8.4 g, 61 mmol, 3.0 equiv) in dioxane (50 mL), H₂O (10 mL) was stirred at 100° C. for 16 h under nitrogen. The resulting mixture was diluted with water (50 mL) and extracted with EtOAc (2×50 mL). The combined organic layers were washed with water (2×50 mL), dried over anhydrous Na₂SO₄, and concentrated under reduced pressure. The crude product was purified using method RPFC1 (10% to 100% gradient in 20 min), resulting in 5-(2-(benzyloxy)-5-fluorophenyl)-1,3-dimethyl-1H-pyrazole (4.0 g, 66%) as a brown solid. m/z: ES⁺ [M+H]⁺=297.25.

### Step 2: 5-(2-(benzyloxy)-5-fluorophenyl)-4-bromo-1,3-dimethyl-1H-pyrazole

A mixture of 5-(2-(benzyloxy)-5-fluorophenyl)-1,3-dimethyl-1H-pyrazole (4.0 g, 13 mmol, 1.0 equiv) and NBS (1.9 g, 11 mmol, 0.8 equiv) in DMF (20 mL) was stirred at room temperature for 16 h under nitrogen. The resulting mixture was diluted with water (50 mL) and extracted with EtOAc (2×50 mL). The combined organic layers were washed with water (2×50 mL), dried over anhydrous Na₂SO₄, and concentrated under reduced pressure. The residue was purified using method RPFC1 (10% to 100% gradient in 20 min), resulting in 5-(2-(benzyloxy)-5-fluorophenyl)-4-bromo-1,3-dimethyl-1H-pyrazole (3.0 g, 59%) as a brown solid. m/z: ES⁺ [M+H]⁺=375.05.

### Step 3: 5-(2-(benzyloxy)-5-fluorophenyl)-1,3-dimethyl-4-(prop-1-en-2-yl)-1H-pyrazole

A mixture of 5-(2-(benzyloxy)-5-fluorophenyl)-4-bromo-1,3-dimethyl-1H-pyrazole (2.0 g, 5.3 mmol, 1.0 equiv), 4,4,5,5-tetramethyl-2-(prop-1-en-2-yl)-1,3,2-dioxaborolane (1.8 g, 11 mmol, 2.0 equiv), Pd(dppf)Cl₂CH₂Cl₂ (0.87 g, 1.1 mmol, 0.2 equiv), and K₂CO₃ (2.2 g, 16 mmol, 3.0 equiv) in dioxane (20 mL), H₂O (5 mL) was stirred at room temperature for 16 h under nitrogen. The resulting mixture was diluted with water (50 mL) and extracted with EtOAc (2×50 mL). The combined organic layers were washed with water (2×50 mL), dried over anhydrous Na₂SO₄, and concentrated under reduced pressure. The crude product was purified using method RPFC1 (10% to 100% gradient in 20 min), resulting in 5-(2-(benzyloxy)-5-fluorophenyl)-1,3-dimethyl-4-(prop-1-en-2-yl)-1H-pyrazole (1.3 g, 73%) as a brown solid. m/z: ES⁺ [M+H]⁺=337.10.

### Step 4: 4-fluoro-2-(4-isopropyl-1,3-dimethyl-1H-pyrazol-5-yl)phenol

A suspension of 5-(2-(benzyloxy)-5-fluorophenyl)-1,3-dimethyl-4-(prop-1-en-2-yl)-1H-pyrazole (1.25 g, 3.7 mmol, 1.0 equiv) and Pd/C (10% wt, wet, 791 mg) in EtOAc (20 mL) was stirred at room temperature for 16 h under hydrogen, and then filtered. The filtrate was concentrated under reduced pressure to give 4-fluoro-2-(4-isopropyl-1,3-dimethyl-1H-pyrazol-5-yl)phenol (570 mg, 62%) as a light-yellow oil. m/z: ES⁺ [M+H]⁺=249.20.

### 4-fluoro-2-(1-isopropyl-3,4-dimethyl-1H-pyrazol-5-yl)phenol

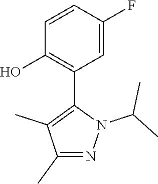 [SMILES: Cc1nn(C(C)C)c(-c2cc(F)ccc2O)c1C]

### Step 1: 5-(2-(benzyloxy)-5-fluorophenyl)-1-isopropyl-3,4-dimethyl-1H-pyrazole

A mixture of 5-chloro-1-isopropyl-3,4-dimethyl-1H-pyrazole (CAS 148066672-s) (1.45 g, 8.4 mmol, 1.0 equiv), (2-(benzyloxy)-5-fluorophenyl)boronic acid (2.1 g, 8.4 mmol, 1.0 equiv), XPhos (600 mg, 1.3 mmol, 0.15 equiv), XPhos Pd G3 (3.3 g, 4.1 mmol, 0.1 equiv), and Cs₂CO₃ (8.2 g, 25 mmol, 3.0 equiv) in toluene (5 mL), H₂O (0.5 mL) was stirred at 90° C. for 16 h under nitrogen. The resulting mixture was diluted with water (20 mL) and extracted with DCM (2×20 mL). The combined organic layers were washed with brine (30 mL), dried over anhydrous Na₂SO₄, and concentrated under reduced pressure. The crude product was purified using method RPFC1 (10% to 100% gradient in 25 min), resulting in 5-(2-(benzyloxy)-5-fluorophenyl)-1-isopropyl-3,4-dimethyl-1H-pyrazole (780 mg, 27%) as a brown oil. m/z: ES⁺ [M+H]⁺=339.05.

### Step 2: 4-fluoro-2-(1-isopropyl-3,4-dimethyl-1H-pyrazol-5-yl)phenol

A suspension of 5-(2-(benzyloxy)-5-fluorophenyl)-1-isopropyl-3,4-dimethyl-1H-pyrazole (510 mg, 1.5 mmol, 1.0 equiv) and Pd/C (10% wt, wet, 500 mg) in MeOH (6 mL) was stirred at room temperature for 4 h under hydrogen and then filtered. The filtrate was concentrated under reduced pressure to give 4-fluoro-2-(1-isopropyl-3,4-dimethyl-1H-pyrazol-5-yl)phenol (370 mg, quant.) as a light-yellow solid. m/z: ES⁺ [M+H]⁺=249.10.

### N-benzyl-N-(5-fluoro-2-hydroxyphenyl)isobutyramide

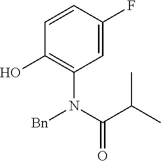 [SMILES: CC(C)C(=O)N(Cc1ccccc1)c1cc(F)ccc1O]

### Step 1: N-(5-fluoro-2-methoxyphenyl)isobutyramide

A mixture of 5-fluoro-2-methoxy aniline (20 g, 142 mmol, 1.0 equiv), NEt₃ (43 g, 425 mmol, 3.0 equiv) and isobutyryl chloride (30 g, 283 mmol, 2.0 equiv) in DCM (200 mL) was stirred at 0° C. for 2 h under nitrogen. The resulting mixture was diluted with water (100 mL) and extracted with DCM (2×100 mL). The combined organic layers were washed with water (2×50 mL), dried over anhydrous Na₂SO₄, and concentrated under reduced pressure. The residue was purified by silica gel column chromatography, eluting with PE/EtOAc (1:1) to afford N-(5-fluoro-2-methoxyphenyl)isobutyramide (25 g, 84%) as an off-white solid. m/z: ES⁺ [M+H]⁺=212.05.

### Step 2: N-benzyl-N-(5-fluoro-2-methoxyphenyl)isobutyramide

A mixture of N-(5-fluoro-2-methoxyphenyl)isobutyramide (10 g, 47 mmol, 1.0 equiv), benzyl bromide (16 g, 95 mmol, 2.0 equiv) and Cs₂CO₃ (46 g, 142 mmol, 3.0 equiv) in DMF (100 mL) was stirred at 80° C. for 16 h under nitrogen. The resulting mixture was diluted with water (50 mL) and extracted with EtOAc (2×50 mL). The combined organic layers were washed with water (2×50 mL), dried over anhydrous Na₂SO₄, and concentrated under reduced pressure. The crude product was purified by RPFC1 (10% to 100% gradient in 20 min) to afford N-benzyl-N-(5-fluoro-2-methoxyphenyl)isobutyramide (10 g, 70%) as a light-yellow oil. m/z: ES⁺ [M+H]⁺=302.15.

### Step 3: N-ethyl-2-(5-fluoro-2-methoxyphenyl)propan-2-amine

A mixture of N-benzyl-N-(5-fluoro-2-methoxyphenyl)isobutyramide (10 g, 33 mmol, 1.0 equiv) in BBr₃ (41.5 g, 166 mmol, 5.0 equiv) in DCM (50 mL) was stirred at 40° C. for 16 h. The reaction was quenched by the addition of MeOH at 0° C. The resulting mixture was concentrated under reduced pressure to give crude N-ethyl-2-(5-fluoro-2-methoxyphenyl)propan-2-amine (6.0 g, 50%) as a brown oil. m/z: ES⁺ [M+H]⁺=288.15.

### 4-fluoro-2-(3-(isopropyl(methyl)amino)oxetan-3-yl)phenol

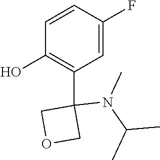 [SMILES: CC(C)N(C)C1(c2cc(F)ccc2O)COC1]

### Step 1: 3-(2-(benzyloxy)-5-fluorophenyl)-N-isopropyl-N-methyloxetan-3-amine

A solution of 3-(2-(benzyloxy)-5-fluorophenyl)-N-isopropyloxetan-3-amine (300 mg, 0.95 mmol, 1.0 equiv), formaldehyde (43 mg, 1.4 mmol, 1.5 equiv), NaBH₃CN (120 mg, 1.9 mmol, 2.0 equiv) in MeOH (1.5 mL) was stirred at room temperature for 16 h. The reaction was quenched with sat. NH₄Cl and extracted with EtOAc (3×100 mL). The combined organic layers were dried over Na₂SO₄ and concentrated under reduced pressure. The residue was purified by RPFC4 (10% to 100% gradient in 10 min) to afford 3-(2-(benzyloxy)-5-fluorophenyl)-N-isopropyl-N-methyloxetan-3-amine (280 mg, 89%) as a colourless solid. m/z: ES⁺ [M+H]⁺=330.15.

### Step 2: 4-fluoro-2-(3-(isopropyl(methyl)amino)oxetan-3-yl)phenol

A solution of 3-(2-(benzyloxy)-5-fluorophenyl)-N-isopropyl-N-methyloxetan-3-amine (300 mg, 0.91 mmol, 1.0 equiv) and Pd/C (194 mg, 10% wt, wet) in MeOH (2 mL) was stirred at room temperature for 4 h under hydrogen. The resulting mixture was filtered, the filter cake washed with DCM (50 mL) and the filtrate concentrated under reduced pressure. The crude product was used in the next step directly without further purification. m/z: ES⁺ [M+H]⁺=240.10.

### N-ethyl-N-(2-(5-fluoro-2-hydroxyphenyl)propan-2-yl)acetamide

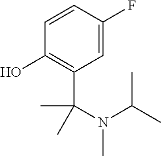 [SMILES: CC(C)N(C)C(C)(C)c1cc(F)ccc1O]

### Step 1: 2-(5-fluoro-2-methoxyphenyl)-2-methylpropanamide

K₂CO₃ (4.5 g, 33 mmol, 1.8 equiv) was added to a solution of 2-(5-fluoro-2-methoxyphenyl)-2-methylpropanenitrile (cAS 1267704-29-9) (3.5 g, 18 mmol, 1.0 equiv) in DMSO (30 mL) at room temperature over 1 min under nitrogen, and then H₂O₂ (10 mL) was added in portions at room temperature. The resulting mixture was stirred at 60° C. for 16 h. The resulting mixture was cooled to room temperature and then extracted with Et₂O (3×20 mL). The combined organic layers were dried over Na₂SO₄ and concentrated under reduced pressure. The residue was purified by RPFC1 (0% to 100% gradient in 10 min) to afford 2-(5-fluoro-2-methoxyphenyl)-2-methylpropanamide (1.0 g, 26%) as a yellow solid. m/z: ES⁺ [M+H]⁺=212.10.

### Step 2: 2-(5-fluoro-2-methoxyphenyl)propan-2-amine

The pH of a solution of 2-(5-fluoro-2-methoxyphenyl)-2-methylpropanamide (900 mg, 4.3 mmol, 1.0 equiv) in MeCN (2 mL) was adjusted to pH-13 with the addition of 5 M aq. NaOH. NaClO (5 mL, 13% aq) was then added in portions over 0.5 min at 0° C., and the resulting mixture was stirred at 50° C. for 2 d. The mixture was then acidified to pH-5 with conc. HCl and extracted with Et₂O (30 mL). The aqueous layer was acidified to pH-9 with 1 M aq. NaOH and extracted with Et₂O (3×20 mL). The combined organic layers were dried over Na₂SO₄, and concentrated under reduced pressure, resulting in 2-(5-fluoro-2-methoxyphenyl)propan-2-amine (200 mg, 26%) as a yellow oil. m/z: ES⁺ [M+H]⁺=184.11.

### Step 3: N-ethyl-2-(5-fluoro-2-methoxyphenyl)propan-2-amine

A mixture of 2-(5-fluoro-2-methoxyphenyl)propan-2-amine (340 mg, 1.9 mmol, 1.0 equiv) and Ti(OEt)₄ (4.2 g, 19 mmol, 10 equiv) and acetaldehyde (5.0 M in THF, 123 mg, 2.8 mmol, 1.5 equiv) in MeOH (4 mL) was stirred at 40° C. for 2 h. NaBH₃CN (233 mg, 3.7 mmol, 2.0 equiv) was then added at room temperature, and the mixture was stirred at 40° C. for 1 h. The resulting mixture was then extracted with Et₂O (3×10 mL), dried over Na₂SO₄, and concentrated under reduced pressure. This resulted in N-ethyl-2-(5-fluoro-2-methoxyphenyl)propan-2-amine (200 mg, 51%) as a yellow oil. m/z: ES⁺ [M+H]⁺=212.14.

### Step 4: N-ethyl-N-(2-(5-fluoro-2-methoxyphenyl)propan-2-yl)acetamide

A mixture of N-ethyl-2-(5-fluoro-2-methoxyphenyl)propan-2-amine (200 mg, 0.95 mmol, 1.0 equiv), NEt₃ (105 mg, 1.0 mmol, 1.1 equiv) and Ac₂O (193 mg, 1.9 mmol, 2.0 equiv) in DCM (2 mL) was stirred at room temperature for 3 h. The resulting mixture was extracted with CHCl₃ (3×5 mL), dried over MgSO₄, and concentrated under reduced pressure. The residue was purified by RPFC1 (0% to 80% gradient in 20 min) to afford N-ethyl-N-(2-(5-fluoro-2-methoxyphenyl)propan-2-yl)acetamide (150 mg, 63%) as a light-yellow oil. m/z: ES⁺ [M+H]⁺=254.15.

### Step 5: N-ethyl-N-(2-(5-fluoro-2-hydroxyphenyl)propan-2-yl)acetamide

A mixture of N-ethyl-N-(2-(5-fluoro-2-methoxyphenyl)propan-2-yl)acetamide (120 mg, 0.47 mmol, 1.0 equiv) in BBr₃ (1 mL, 1 M in DCM) and DCM (0.1 mL) was stirred at room temperature for 3 h. The reaction was quenched by the addition of sat. NaHCO₃ (aq.) (10 mL) at 0° C. The resulting mixture was extracted with Et₂O (3×10 mL), dried over anhydrous Na₂SO₄, and concentrated under reduced pressure. This resulted in N-ethyl-N-(2-(5-fluoro-2-hydroxyphenyl)propan-2-yl)acetamide (110 mg, 97%) as a light-yellow solid. m/z: ES⁺ [M+H]⁺=240.13.

### 2-(3-(dimethylamino)oxetan-3-yl)-4-fluorophenol

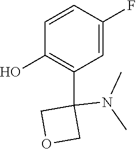

### Step 1: 3-(2-(benzyloxy)-5-fluorophenyl)-N,N-dimethyloxetan-3-amine

A solution of 3-(2-(benzyloxy)-5-fluorophenyl)oxetan-3-amine (250 mg, 0.92 mmol, 1.0 equiv), formaldehyde (110 mg, 3.7 mmol, 4.0 equiv), NaBH₃CN (115 mg, 1.9 mmol, 2.0 equiv) in MeOH (5 mL) was stirred at room temperature for 16 h. The reaction was quenched with sat. NH₄Cl and extracted with EtOAc (3×100 mL). The combined organic layers were dried over Na₂SO₄ and concentrated under reduced pressure. The residue was purified by RPFC1 (10% to 100% gradient in 10 min) to afford 3-(2-(benzyloxy)-5-fluorophenyl)-N,N-dimethyloxetan-3-amine (200 mg, 73%) as a brown solid. m/z: ES⁺ [M+H]⁺=302.05.

### Step 2: 2-(3-(dimethylamino)oxetan-3-yl)-4-fluorophenol

A suspension of 3-(2-(benzyloxy)-5-fluorophenyl)-N,N-dimethyloxetan-3-amine (200 mg, 0.66 mmol, 1.0 equiv) and Pd/C (141 mg, 1.3 mmol, 2.0 equiv) in MeOH (5 mL) was stirred at room temperature for 2 h under hydrogen. The resulting mixture was filtered, the filter cake washed with DCM (100 mL) and the filtrate concentrated under reduced pressure, resulting in 2-(3-(dimethylamino)oxetan-3-yl)-4-fluorophenol (120 mg, 86%) as a brown solid. m/z: ES⁺ [M+H]⁺=212.10.

### 4-fluoro-2-(5-isopropyl-4-methyl-1H-imidazol-1-yl)phenol

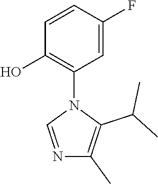 [SMILES: Cc1ncn(-c2cc(F)ccc2O)c1C(C)C]

### Step 1: 1-(2-(benzyloxy)-5-fluorophenyl)-4-methyl-1H-imidazole

A solution of (2-(benzyloxy)-5-fluorophenyl)boronic acid (5.5 g, 23 mmol, 1.0 equiv) and 4-methyl-1H-imidazole (2.2 g, 27 mmol, 1.2 equiv), Cu(OAc)₂ (2.1 g, 11 mmol, 0.5 equiv) and NEt₃ (6.8 g, 68 mmol, 3.0 equiv) in MeOH (55 mL) was stirred at room temperature for 24 h under air. After filtration, the filtrate was concentrated under reduced pressure, and the residue purified by silica gel chromatography, eluting with PE/EtOAc (5:2), resulting in 1-(2-(benzyloxy)-5-fluorophenyl)-4-methyl-1H-imidazole (2.8 g, 44%) as a brown gum. m/z: ES⁺ [M+H]⁺=283.05.

### Step 2: 1-(2-(benzyloxy)-5-fluorophenyl)-5-bromo-4-methyl-1H-imidazole

A solution of 1-(2-(benzyloxy)-5-fluorophenyl)-4-methyl-1H-imidazole (2.7 g, 9.6 mmol, 1.0 equiv) and NBS (1.7 g, 9.6 mmol, 1.0 equiv) in MeCN (50 mL) was stirred at room temperature for 3 h. The resulting mixture was then concentrated under reduced pressure, and the resulting residue was purified using method RPFC1 (10% to 100% gradient in 25 min), to afford 1-(2-(benzyloxy)-5-fluorophenyl)-5-bromo-4-methyl-1H-imidazole (1.6 g, 46%) as a brown oil. m/z: ES⁺ [M+H]⁺=360.95.

### Step 3: 1-(2-(benzyloxy)-5-fluorophenyl)-4-methyl-5-(prop-1-en-2-yl)-1H-imidazole

A solution of 1-(2-(benzyloxy)-5-fluorophenyl)-5-bromo-4-methyl-1H-imidazole (1.6 g, 4.4 mmol, 1.0 equiv), 4,4,5,5-tetramethyl-2-(prop-1-en-2-yl)-1,3,2-dioxaborolane (1.5 g, 8.7 mmol, 2.0 equiv), Pd(dppf)Cl₂CH₂Cl₂ (890 mg, 1.1 mmol, 0.25 equiv), and K₂CO₃ (1.2 g, 8.7 mmol, 2.0 equiv) in dioxane (10 mL)/water (1 mL) was stirred at 100° C. for 16 h under nitrogen. The mixture was allowed to warm to room temperature and then poured into water (20 mL) and extracted with DCM (3×20 mL). The combined organic layers were washed with brine (2×20 mL) dried over Na₂SO₄ and concentrated under reduced pressure. The residue was purified by RPFC1 (10% to 100% gradient in 30 min), resulting in 1-(2-(benzyloxy)-5-fluorophenyl)-4-methyl-5-(prop-1-en-2-yl)-1H-imidazole (569 mg, 41%) as a brown oil. m/z: ES⁺ [M+H]⁺=323.10.

### Step 4: 4-fluoro-2-(5-isopropyl-4-methyl-1H-imidazol-1-yl)phenol

A solution of 1-(2-(benzyloxy)-5-fluorophenyl)-4-methyl-5-(prop-1-en-2-yl)-1H-imidazole (568 mg, 1.8 mmol, 1.0 equiv) and Pd/C (0.6 g, 10% wt, wet) in MeOH (10 mL) was stirred at room temperature for 24 h under hydrogen. The resulting mixture was then filtered, and the filtrate was concentrated under reduced pressure. This resulted in 4-fluoro-2-(5-isopropyl-4-methyl-1H-imidazol-1-yl)phenol (360 mg, quant.) as a yellow oil. m/z: ES⁺ [M+H]⁺=235.00.

### 4-fluoro-2-(2-isopropyl-4-methyl-1H-imidazol-1-yl)phenol

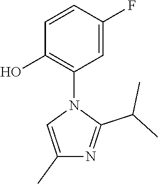 [SMILES: Cc1cn(-c2cc(F)ccc2O)c(C(C)C)n1]

### Step 1: 4,5-dibromo-1-(5-fluoro-2-methoxyphenyl)-2-isopropyl-1H-imidazole

A solution of 1-(5-fluoro-2-methoxyphenyl)-2-isopropylimidazole (cAS 2169922-00-1) (3.5 g, 15 mmol, 1.0 equiv) and NBS (9.3 g, 53 mmol, 3.5 equiv) in DMF (35 mL) was stirred at room temperature for 5 h. The resulting mixture was diluted with water (100 mL) and extracted with EtOAc (3×100 mL). The combined organic layers were washed with brine (2×100 mL) dried over Na₂SO₄ and concentrated under reduced pressure. The residue was purified by RPFC1 (10% to 100% gradient in 20 min), resulting in 4,5-dibromo-1-(5-fluoro-2-methoxyphenyl)-2-isopropyl-1H-imidazole (2.1 g, 36%) as a yellow semi-solid. m/z: ES⁺ [M+H]⁺=392.10.

### Step 2: 4-bromo-1-(5-fluoro-2-methoxyphenyl)-2-isopropyl-1H-imidazole

n-BuL₁ (2.5 M in THF, 2.2 mL, 5.4 mmol, 1.0 equiv) was added dropwise and at −75° C. to a solution of 4,5-dibromo-1-(5-fluoro-2-methoxyphenyl)-2-isopropyl-1H-imidazole (2.1 g, 5.4 mmol, 1.0 equiv) in THF (21 mL). The mixture was stirred at 0° C. for 2 h under nitrogen and then quenched with sat. NH₄Cl (aq.) at 0° C. and extracted with EtOAc (3×100 mL). The combined organic layers were washed with brine (2×100 mL), dried over Na₂SO₄, and concentrated under reduced pressure. The residue was purified by RPFC1 (10% to 100% gradient in 20 min), to afford 4-bromo-1-(5-fluoro-2-methoxyphenyl)-2-isopropyl-1H-imidazole (1.1 g, 66%) as a yellow solid. m/z: ES⁺ [M+H]⁺=315.05.

### Step 3: 1-(5-fluoro-2-methoxyphenyl)-2-isopropyl-4-methyl-1H-imidazole

A solution of 4-bromo-1-(5-fluoro-2-methoxyphenyl)-2-isopropyl-1H-imidazole (1.1 g, 3.5 mmol, 1.0 equiv), methylboronic acid (0.74 g, 12 mmol, 3.5 equiv), Pd₂(dba)₃ (0.32 g, 0.35 mmol, 0.1 equiv), XPhos (0.25 g, 0.53 mmol, 0.15 equiv) and tBuONa (1.0 g, 11 mmol, 3 equiv) in dioxane (11 mL) was stirred at 100° C. for 16 h under nitrogen. The mixture was allowed to warm to room temperature, and diluted with water (50 mL), then extracted with DCM (3×50 mL). The combined organic layers were washed with brine (2×100 mL), dried over Na₂SO₄, and concentrated under reduced pressure. The residue was purified by RPFC1 (10% to 100% gradient in 20 min), resulting in 1-(5-fluoro-2-methoxyphenyl)-2-isopropyl-4-methyl-1H-imidazole (400 mg, 46%) as a yellow solid. m/z: ES⁺ [M+H]⁺=249.20.

### Step 4: 4-fluoro-2-(2-isopropyl-4-methyl-1H-imidazol-1-yl)phenol

A mixture of 1-(5-fluoro-2-methoxyphenyl)-2-isopropyl-4-methyl-1H-imidazole (400 mg, 1.6 mmol, 1.0 equiv) in BBr₃ (1 M in DCM, 6.4 mL, 6.4 mmol, 4.0 equiv) and DCM (1 mL) was stirred at room temperature for 16 h. The reaction was quenched by the addition MeOH at 0° C. The resulting mixture was concentrated under reduced pressure, triturated with EtOAc (10 mL), and filtered. The solid was washed with EtOAc (3×5 mL), resulting in 4-fluoro-2-(2-isopropyl-4-methyl-1H-imidazol-1-yl)phenol (130 mg, 34%) as an off-white solid. m/z: ES⁺ [M+H]⁺=235.05.

### 4-fluoro-2-(4-isopropyl-6-methylpyrimidin-5-yl)phenol

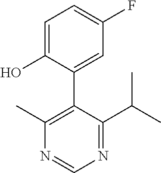 [SMILES: Cc1ncnc(C(C)C)c1-c1cc(F)ccc1O]

A mixture of 5-bromo-4-isopropyl-6-methylpyrimidine (cAS 1817776-83-2) (1.0 g, 4.6 mmol, 1.0 equiv), 5-fluoro-2-hydroxy phenylboronic acid (2.2 g, 14 mmol, 3.0 equiv), Pd(PPh₃)₄ (0.54 mg, 0.47 mmol, 0.1 equiv), and K₃PO₄ (2.0 g, 9.3 mmol, 2.0 equiv) in dioxane (10 mL)/H₂O (2 mL) was stirred at 100° C. for 16 h under nitrogen. The mixture was diluted with water (20 mL) and extracted with DCM (3×15 mL). The combined organic layers were dried over anhydrous Na₂SO₄ and concentrated under reduced pressure. The residue was purified using method RPFC1 (10% to 100% gradient in 20 min), to afford 4-fluoro-2-(4-isopropyl-6-methylpyrimidin-5-yl)phenol (650 mg, 57%) as a colourless solid. m/z: ES⁺ [M+H]⁺=247.12.

### 2-(ethyl(3-isopropyloxetan-3-yl)amino)-4-fluorophenol

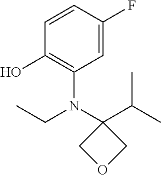 [SMILES: CCN(c1cc(F)ccc1O)C1(C(C)C)COC1]

### Step 1: N-(2-(benzyloxy)-5-fluorophenyl)-3-isopropyloxetan-3-amine

A solution of 2-(benzyloxy)-5-fluoroaniline (3.5 g, 16 mmol, 1.0 equiv) and oxetan-3-one (2.5 g, 35 mmol, 2.2 equiv), 2-iodopropane (8.5 g, 50 mmol, 3.1 equiv), HFIP (5.3 g, 31 mmol, 1.9 equiv) in DCM (100 mL) was stirred at room temperature for 48 h. The resulting mixture was diluted with water (50 mL) and extracted with DCM (3×50 mL). The combined organic layers were washed with brine (20 mL), dried over anhydrous Na₂SO₄, and concentrated under reduced pressure. The residue was purified by RPFC1 (30% to 70% gradient in 10 min) to afford N-(2-(benzyloxy)-5-fluorophenyl)-3-isopropyloxetan-3-amine (900 mg, 18%) as a yellow oil. m/z: ES⁺ [M+H]⁺=316.15.

### Step 2: N-(2-(benzyloxy)-5-fluorophenyl)-N-ethyl-3-isopropyloxetan-3-amine

A solution of N-(2-(benzyloxy)-5-fluorophenyl)-3-isopropyloxetan-3-amine (400 mg, 1.3 mmol, 1.0 equiv) and iodoethane (400 mg, 2.6 mmol, 2.0 equiv), NaH (120 mg, 5.0 mmol, 3.9 equiv) in DMF (4 mL) was stirred at room temperature for 16 h under nitrogen. The resulting mixture was diluted with water (10 mL) and extracted with EtOAc (3×10 mL). The combined organic layers were washed with brine (2×10 mL), dried over Na₂SO₄, and concentrated under reduced pressure. The residue was purified by Prep-TLC (PE/EtOAc 1:1) to afford N-(2-(benzyloxy)-5-fluorophenyl)-N-ethyl-3-isopropyloxetan-3-amine (300 mg, 69%) as a yellow oil. m/z: ES⁺ [M+H]⁺=344.25.

### Step 3: 2-(ethyl(3-isopropyloxetan-3-yl)amino)-4-fluorophenol

A suspension of N-(2-(benzyloxy)-5-fluorophenyl)-N-ethyl-3-isopropyloxetan-3-amine (290 mg, 0.84 mmol, 1.0 equiv) and Pd/C (100 mg, 10% wt, wet) in EtOAc (5 mL) was stirred at room temperature for 16 h under hydrogen. The resulting mixture was filtered and the filter cake was washed with EtOAc (4×10 mL). The filtrate was concentrated under reduced pressure to afford 2-(ethyl(3-isopropyloxetan-3-yl)amino)-4-fluorophenol (140 mg, 65%) as a yellow oil. m/z: ES⁺ [M+H]⁺=254.20.

### 4-(5-fluoro-2-hydroxyphenyl)-3-isopropyl-1,5-dimethylpyridin-2(1H)-one

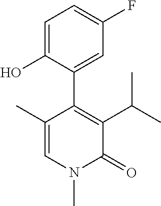 [SMILES: Cc1cn(C)c(=O)c(C(C)C)c1-c1cc(F)ccc1O]

### Step 1: 2-fluoro-4-iodo-5-methyl-3-(prop-1-en-2-yl)pyridine

A solution of 2-(2-fluoro-4-iodo-5-methylpyridin-3-yl)propan-2-ol(cAS 153034-94-7) (860 mg, 2.9 mmol, 1.0 equiv) and Et₃SiH (1.7 g, 15 mmol, 5.0 equiv) in TFA (5 mL) was stirred at 90° C. for 2 h under nitrogen. The resulting mixture was concentrated under reduced pressure. The residue was purified by RPFC1 (10% to 70% gradient in 10 min) to afford 2-fluoro-4-iodo-5-methyl-3-(prop-1-en-2-yl)pyridine (380 mg, 47%) was obtained as a yellow oil. m/z: ES⁺ [M+H]⁺=278.05.

### Step 2: 2-fluoro-4-(5-fluoro-2-methoxyphenyl)-5-methyl-3-(prop-1-en-2-yl)pyridine

A mixture of 2-fluoro-4-iodo-5-methyl-3-(prop-1-en-2-yl)pyridine (360 mg, 1.3 mmol, 1.0 equiv), (5-fluoro-2-methoxyphenyl)boronic acid (220 mg, 1.3 mmol, 1.0 equiv), Pd(dppf)Cl₂CH₂Cl₂ (110 mg, 0.14 mmol, 0.1 equiv), and K₂CO₃ (550 mg, 4.0 mmol, 3.0 equiv) in dioxane (10 mL) and H₂O (2 mL) was stirred at 60° C. for 16 h under nitrogen. The mixture was diluted with water (10 mL) and extracted with EtOAc (3×20 mL). The combined organic layers were washed with brine (10 mL), dried over anhydrous Na₂SO₄ and concentrated under reduced pressure. The residue was purified using method RPFC1 (40% to 70% gradient in 10 min), to afford 2-fluoro-4-(5-fluoro-2-methoxyphenyl)-5-methyl-3-(prop-1-en-2-yl)pyridine (300 mg, 84%) as a yellow solid. m/z: ES⁺ [M+H]⁺=276.15.

### Step 3: 2-fluoro-4-(5-fluoro-2-methoxyphenyl)-3-isopropyl-5-methylpyridine

A solution of 2-fluoro-4-(5-fluoro-2-methoxyphenyl)-5-methyl-3-(prop-1-en-2-yl)pyridine (290 mg, 1.1 mmol, 1.0 equiv) and Pd/C (59 mg, 0.55 mmol, 0.52 equiv) in MeOH (2 mL) was stirred at room temperature for 16 h under hydrogen. The resulting mixture was filtered, and the filter cake was washed with MeOH (3×10 mL). The filtrate was concentrated under reduced pressure, resulting in 2-fluoro-4-(5-fluoro-2-methoxyphenyl)-3-isopropyl-5-methylpyridine (170 mg, 58%) as a yellow oil. m/z: ES⁺ [M+H]⁺=278.15.

### Step 4: 4-(5-fluoro-2-methoxyphenyl)-3-isopropyl-5-methylpyridin-2(1H)-one

A solution of 2-fluoro-4-(5-fluoro-2-methoxyphenyl)-3-isopropyl-5-methylpyridine (250 mg, 0.90 mmol, 1.0 equiv) and water (0.4 mL) in AcOH (2 mL) was stirred at 120° C. for 48 h. The resulting mixture was concentrated under reduced pressure, and the residue purified by RPFC1 (20% to 80% gradient in 10 min), to afford 4-(5-fluoro-2-methoxy phenyl)-3-isopropyl-5-methylpyridin-2(1H)-one (130 mg, 52%) as a yellow oil. m/z: ES⁺ [M+H]⁺=276.15.

### Step 5: 4-(5-fluoro-2-methoxyphenyl)-3-isopropyl-1,5-dimethylpyridin-2(1H)-one

A solution of 4-(5-fluoro-2-methoxyphenyl)-3-isopropyl-5-methylpyridin-2(1H)-one (120 mg, 0.44 mmol, 1.0 equiv) and Cs₂CO₃ (570 mg, 1.7 mmol, 4.0 equiv), MeI (150 mg, 1.0 mmol, 2.4 equiv) in DMF (3 mL) was stirred at room temperature for 3 h under nitrogen. The resulting mixture was diluted with water (10 mL) and extracted with EtOAc (3×10 mL). The combined organic layers were washed with brine (2×5 mL), dried over anhydrous Na₂SO₄ and concentrated under reduced pressure. The residue was purified using method RPFC1 (40% to 70% gradient in 10 min), to give 4-(5-fluoro-2-methoxyphenyl)-3-isopropyl-1,5-dimethylpyridin-2(1H)-one (100 mg, 79%) as a yellow oil. m/z: ES⁺ [M+H]⁺=290.20.

### Step 6: 4-(5-fluoro-2-hydroxyphenyl)-3-isopropyl-1,5-dimethylpyridin-2(1H)-one

A solution of 4-(5-fluoro-2-methoxyphenyl)-3-isopropyl-1,5-dimethylpyridin-2(1H)-one (90 mg, 0.31 mmol, 1.0 equiv) and BBr₃ (1 M in DCM, 2 mL, 2.0 mmol, 6.0 equiv) in DCM (2 mL) was stirred at room temperature for 16 h under nitrogen. The reaction was quenched by the addition of MeOH (5 mL) and concentrated under reduced pressure. The residue was purified using method RPFC1 (30% to 70% gradient in 10 min), to give 4-(5-fluoro-2-hydroxyphenyl)-3-isopropyl-1,5-dimethylpyridin-2(1H)-one (60 mg, 70%) as a yellow oil. m/z: ES⁺ [M+H]⁺=276.20.

### 4-(5-fluoro-2-hydroxyphenyl)-5-isopropyl-1,3-dimethylpyridin-2(1H)-one

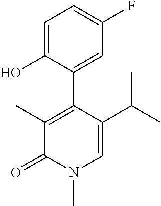 [SMILES: Cc1c(-c2cc(F)ccc2O)c(C(C)C)cn(C)c1=O]

### Step 1: 2-fluoro-3-iodo-5-isopropylpyridine

A solution of 2-fluoro-5-isopropylpyridine (1 g, 7.2 mmol, 1.0 equiv.) and LDA (0.77 g, 7.2 mmol, 1.0 equiv.) in THF (10 mL) was stirred at −78° C. for 2 h under nitrogen, and then added to a solution of iodine (1.8 g, 7.2 mmol, 1.0 equiv.) in THF (10 mL) −78° C. and stirred for 2 h under nitrogen. The resulting mixture was diluted with water (30 mL) and extracted with DCM (2×50 mL). The combined organic layers were washed with brine (30 mL) and dried over anhydrous Na₂SO₄. After filtration, the residue was purified by RPFC1 (10% to 100% gradient in 10 min), resulting in 2-fluoro-3-iodo-5-isopropylpyridine (200 mg, 11%) as a brown oil. m/z: ES⁺ [M+H]⁺=265.25.

### Step 2: 2-fluoro-4-iodo-5-isopropyl-3-methylpyridine

A solution of 2-fluoro-3-iodo-5-isopropylpyridine (300 mg, 1.1 mmol, 1.0 equiv.) and LDA (242 mg, 2.3 mmol, 2.0 equiv.) in THF (3 mL) was stirred at −78° C. for 2 h under nitrogen. Then a solution of MeI (482 mg, 3.4 mmol, 3.0 equiv.) in THF (2 mL) was added at −78° C., and the mixture was stirred for 2 h under nitrogen. The resulting mixture was diluted with water (30 mL) and extracted with DCM (2×50 mL). The combined organic layers were washed with brine (30 mL), dried over anhydrous Na₂SO₄, and concentrated. The residue was purified by RPFC1 (10% to 100% gradient in 10 min), resulting in 2-fluoro-4-iodo-5-isopropyl-3-methylpyridine (40 mg, 13%) as a brown oil. m/z: ES⁺ [M+H]⁺=280.15.

### Step 3: 4-iodo-5-isopropyl-3-methylpyridin-2(1H)-one

A solution of 2-fluoro-4-iodo-5-isopropyl-3-methylpyridine (290 mg, 1.0 mmol, 1.0 equiv.) in AcOH (6 mL) and H₂O (2 mL) was stirred at 80° C. for 16 h. The reaction was quenched with sat. NH₄Cl (aq.) at room temperature. The resulting mixture was extracted with EtOAc (3×100 mL), dried over anhydrous Na₂SO₄, and concentrated. The residue was purified by RPFC1 (10% to 100% gradient in 10 min), resulting in 4-iodo-5-isopropyl-3-methylpyridin-2(1H)-one (200 mg, 69%) as a brown oil. m/z: ES⁺ [M+H]⁺=278.15.

### Step 4: 4-iodo-5-isopropyl-1,3-dimethylpyridin-2(1H)-one

A mixture of NaH (33 mg, 1.4 mmol, 2.0 equiv.) and 4-iodo-5-isopropyl-3-methylpyridin-2(1H)-one (190 mg, 0.69 mmol, 1.0 equiv.) in DMF (5 mL) was stirred at room temperature for 1 h. MeI (195 mg, 1.4 mmol, 2.0 equiv.) was then added and stirring continued at room temperature for 1 h. The resulting mixture was diluted with water (30 mL) and extracted with DCM (2×50 mL). The combined organic layers were washed with brine (30 mL), dried over anhydrous Na₂SO₄, and concentrated. The residue was purified by RPFC2 (10% to 50% gradient in 10 min), resulting in 4-iodo-5-isopropyl-1,3-dimethylpyridin-2(1H)-one (180 mg, 90%) as a brown oil. m/z: ES⁺ [M+H]⁺=291.95.

### Step 5: 4-(2-(benzyloxy)-5-fluorophenyl)-5-isopropyl-1,3-dimethylpyridin-2(11)-one

A mixture of 4-iodo-5-isopropyl-1,3-dimethylpyridin-2(1H)-one (150 mg, 0.52 mmol, 1.0 equiv), 2-(benzyloxy)-5-fluorophenylboronic acid (190 mg, 0.77 mmol, 1.5 equiv.), Pd₂(dba)₃ (71 mg, 0.08 mmol, 0.15 equiv.), XPhos (37 mg, 0.08 mmol, 0.15 equiv.), and K₂CO₃ (152, 1.0 mmol, 2.0 equiv) in dioxane (5 mL) was stirred at 60° C. for 16 h under nitrogen. The reaction was quenched with sat. NH₄Cl (aq.) and extracted with EtOAc (3×100 mL). The combined organic layers were washed with brine (10 mL), dried over anhydrous Na₂SO₄ and concentrated under reduced pressure. The residue was purified using method RPFC1 (10% to 100% gradient in 10 min), to afford 4-(2-(benzyloxy)-5-fluorophenyl)-5-isopropyl-1,3-dimethylpyridin-2(1H)-one (90 mg, 48%) as a yellow oil. m/z: ES⁺ [M+H]⁺=366.15.

### Step 6: 4-(5-fluoro-2-hydroxyphenyl)-5-isopropyl-1,3-dimethylpyridin-2(1H)-one

A suspension of 4-(2-(benzyloxy)-5-fluorophenyl)-5-isopropyl-1,3-dimethylpyridin-2(1H)-one (140 mg, 0.38 mmol, 1.0 equiv) and Pd/C (82 mg, 10% wt, wet) in MeOH (5 mL) was stirred at room temperature for 3 h under hydrogen. The resulting mixture was filtered, the filter cake was washed with MeOH (5 mL). The filtrate was concentrated under reduced pressure to afford 4-(2-(benzyloxy)-5-fluorophenyl)-5-isopropyl-1,3-dimethylpyridin-2(1H)-one (90 mg, 85%) as a brown solid. m/z: ES⁺ [M+H]⁺=276.10. 6-(5-fluoro-2-hydroxyphenyl)-5-isopropyl-1-methylpyrimidin-2(1H)-one

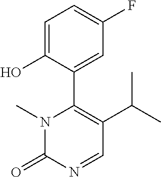 [SMILES: CC(C)c1cnc(=O)n(C)c1-c1cc(F)ccc1O]

### Step 1: 1-(5-fluoro-2-methoxyphenyl)-3-methylbutan-1-one

A solution of AlCl₃ (13.7 g, 103 mmol, 1.3 equiv) and 1-fluoro-4-methoxybenzene (10 g, 79 mmol, 1.0 equiv) in DCM (100 mL) was stirred at 0° C. for 15 min under nitrogen. 3-Methylbutanoyl chloride (11.5 g, 95 mmol, 1.2 equiv) was then added dropwise at 0° C. under nitrogen. The resulting mixture was allowed to stir at room temperature for 16 h under nitrogen. The reaction was quenched with sodium bicarbonate at 0° C. and extracted with DCM (3×100 mL). The combined organic layers were washed with brine (2×100 mL), dried over anhydrous Na₂SO₄, and concentrated. The residue was purified by silica gel column chromatography, eluting with PE/EtOAc (2:3) to afford 1-(5-fluoro-2-methoxyphenyl)-3-methylbutan-1-one (12.5 g, 75%) as a yellow oil. m/z: ES⁺ [M+H]⁺=211.05.

### Step 2: (E)-2-((dimethylamino)methylene)-1-(5-fluoro-2-methoxyphenyl)-3-methylbutan-1-one

A solution of 1-(5-fluoro-2-methoxyphenyl)-3-methylbutan-1-one (12 g, 57 mmol, 1.0 equiv) in DMF-DMA (120 mL) was stirred at 130° C. for 16 h. The resulting mixture was concentrated under reduced pressure to give crude (E)-2-((dimethylamino)methylene)-1-(5-fluoro-2-methoxyphenyl)-3-methylbutan-1-one, which was used in the next step directly without further purification. m/z: ES⁺ [M+H]⁺=266.05.

### Step 3: 6-(5-fluoro-2-methoxyphenyl)-5-isopropyl-1-methylpyrimidin-2(1H)-one

A solution of (E)-2-((dimethylamino)methylene)-1-(5-fluoro-2-methoxyphenyl)-3-methylbutan-1-one (10.7 g, 40 mmol, 1.0 equiv), methylurea (3.0 g, 40 mmol, 1.0 equiv) and TsOH (6.9 g, 40 mmol, 1.0 equiv) in dioxane (110 mL) was stirred at 115° C. for 16 h. The mixture was diluted with water (200 mL) and extracted with DCM (3×100 mL). The combined organic layers were washed with brine (3×100 mL), dried over anhydrous Na₂SO₄ and concentrated under reduced pressure. The residue was purified by RPFC2 (10% to 100% gradient in 25 min), resulting in 6-(5-fluoro-2-methoxyphenyl)-5-isopropyl-1-methylpyrimidin-2(1H)-one (800 mg, 7%) as a light orange solid. m/z: ES⁺ [M+H]⁺=277.05.

### Step 4: 6-(5-fluoro-2-hydroxyphenyl)-5-isopropyl-1-methylpyrimidin-2(1H)-one

A solution of 6-(5-fluoro-2-methoxyphenyl)-5-isopropyl-1-methylpyrimidin-2(1H)-one (400 mg, 1.5 mmol, 1.0 equiv) and BBr₃ (1 M in DCM, 4 mL, 4.0 mmol, 2.5 equiv) in DCM (4 mL) was stirred at room temperature for 16 h under nitrogen. The reaction was quenched by the addition MeOH at 0° C., and concentrated under reduced pressure. The product was precipitated by the addition of EtOAc (20 mL). The precipitated solids were collected by filtration and washed with EtOAc (20 mL). This resulted in 6-(5-fluoro-2-hydroxyphenyl)-5-isopropyl-1-methylpyrimidin-2(1H)-one (300 mg, quant.) as a light-yellow solid. m/z: ES⁺ [M+H]⁺=263.05.

### 5-(5-fluoro-2-hydroxyphenyl)-6-isopropyl-1-methylpyrazin-2(1H)-one

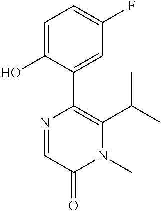 [SMILES: CC(C)c1c(-c2cc(F)ccc2O)ncc(=O)n1C]

### Step 1: 1-(benzyloxy)-4-fluoro-2-(3-methylbut-1-yn-1-yl)benzene

A solution of 3-methylbut-1-yne (2.0 g, 29 mmol, 1.0 equiv) and 1-(benzyloxy)-2-bromo-4-fluorobenzene (4.0 g, 14 mmol, 0.5 equiv), Pd(PPh₃)₄ (3.4 g, 2.9 mmol, 0.1 equiv), CuI (620 mg, 3.3 mmol, 0.11 equiv), NEt₃ (9.0 g, 89 mmol, 3.0 equiv) in THF (30 mL) was stirred at room temperature for 16 h under nitrogen. The resulting mixture was diluted with water (10 mL) and extracted with EtOAc (3×20 mL). The combined organic layers were washed with brine (10 mL), dried over anhydrous Na₂SO₄, and concentrated under reduced pressure. The residue was purified by RPFC1 (10% to 50% gradient in 10 min), to give 1-(benzyloxy)-4-fluoro-2-(3-methylbut-1-yn-1-yl)benzene (2.0 g, 25%) as a yellow oil. m/z: ES⁺ [M+H]⁺=267.10.

### Step 2: 1-(2-(benzyloxy)-5-fluorophenyl)-3-methylbutane-1,2-dione

A solution of 1-(benzyloxy)-4-fluoro-2-(3-methylbut-1-yn-1-yl)benzene (1.0 g, 3.7 mmol, 1.0 equiv) and KMnO₄ (2.4 g, 15 mmol, 4.1 equiv), MgSO₄ (600 mg, 5.0 mmol, 1.3 equiv), Na₂CO₃ (600 mg, 5.7 mmol, 1.5 equiv) in acetone (40 mL)/water (25 mL) was stirred at room temperature for 16 h under nitrogen. The resulting mixture was diluted with water (20 mL) and extracted with EtOAc (3×20 mL). The combined organic layers were washed with brine (2×5 mL), dried over anhydrous Na₂SO₄, and was concentrated under reduced pressure. The residue was purified by silica gel column chromatography, eluting with PE/EtOAc (5:1) to afford 1-(2-(benzyloxy)-5-fluorophenyl)-3-methyl butane-1,2-dione (800 mg, 71%) as a yellow solid. m/z: ES⁺ [M+H]⁺=299.10.

### Step 3: 5-(2-(benzyloxy)-5-fluorophenyl)-6-isopropylpyrazin-2(1H)-one

A solution of 1-(2-(benzyloxy)-5-fluorophenyl)-3-methylbutane-1,2-dione (780 mg, 2.6 mmol, 1.0 equiv), 2-aminoacetamide (230 mg, 3.1 mmol, 1.2 equiv) and NaOH (230 mg, 5.8 mmol, 2.2 equiv) in MeOH (10 mL) was stirred at room temperature for 16 h. The resulting mixture was diluted with water (10 mL) and extracted with EtOAc (3×30 mL). The combined organic layers were washed with brine (2×5 mL), dried over anhydrous Na₂SO₄, and concentrated under reduced pressure. The residue was purified by RPFC1 (10% to 50% gradient in 10 min), to give 5-(2-(benzyloxy)-5-fluorophenyl)-6-isopropylpyrazin-2(1H)-one (800 mg, 91%) as a yellow oil. m/z: ES⁺ [M+H]⁺=339.20.

### Step 4: 5-(2-(benzyloxy)-5-fluorophenyl)-6-isopropyl-1-methylpyrazin-2(11)-one

A solution of 5-(2-(benzyloxy)-5-fluorophenyl)-6-isopropylpyrazin-2(1H)-one (450 mg, 1.3 mmol, 1.0 equiv), NaH (135 mg, 5.6 mmol, 4.2 equiv) and MeI (500 mg, 3.5 mmol, 2.7 equiv) in DMF (5 mL) was stirred at room temperature for 2 h under nitrogen. The resulting mixture was diluted with water (30 mL) and extracted with EtOAc (3×30 mL). The combined organic layers were washed with brine (2×5 mL), dried over anhydrous Na₂SO₄, and concentrated under reduced pressure. The residue was purified by RPFC1 (20% to 70% gradient in 10 min), to give 5-(2-(benzyloxy)-5-fluorophenyl)-6-isopropyl-1-methylpyrazin-2(1H)-one (280 mg, 60%) was obtained as a yellow oil. m/z: ES⁺ [M+H]⁺=351.00.

### Step 5: 5-(5-fluoro-2-hydroxyphenyl)-6-isopropyl-1-methylpyrazin-2(1H)-one

A suspension of 5-(2-(benzyloxy)-5-fluorophenyl)-6-isopropyl-1-methylpyrazin-2(1H)-one (270 mg, 0.77 mmol, 1.0 equiv) and Pd/C (40 mg, 10% wt, wet) in MeOH (5 mL) was stirred at room temperature for 16 h under hydrogen. The resulting mixture was filtered, and the filter cake was washed with MeOH (3×20 mL). The filtrate was concentrated under reduced pressure to afford 5-(5-fluoro-2-hydroxyphenyl)-6-isopropyl-1-methylpyrazin-2(1H)-one (160 mg, 80%) as a brown solid. m/z: ES⁺ [M+H]⁺=263.010.

### 4-fluoro-2-(5-isopropyl-2,4-dimethyl-1H-imidazol-1-yl)phenol

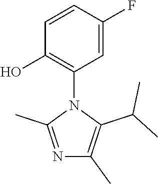 [SMILES: Cc1nc(C)n(-c2cc(F)ccc2O)c1C(C)C]

### Step 1: 1-(2-(benzyloxy)-5-fluorophenyl)-2,4-dimethyl-1H-imidazole

A mixture of 2,4-dimethyl-1H-imidazole (6.0 g, 62 mmol, 1.0 equiv), (2-(benzyloxy)-5-fluorophenyl)boronic acid (12.3 g, 25 mmol, 0.8 equiv), Cu(OAc)₂ (1.1 g, 6.2 mmol, 0.2 equiv) and NEt₃ (18.9 g, 187 mmol, 3.0 equiv) in MeOH (60 mL) was stirred at room temperature for 24 h. The resulting mixture was diluted with water (100 mL) and extracted with EtOAc (3×100 mL). The combined organic layers were washed with brine (2×100 mL), dried over anhydrous Na₂SO₄, and concentrated under reduced pressure. The crude product was purified by silica gel column chromatography, eluting with PE/EtOAc (1:10), resulting in 1-(2-(benzyloxy)-5-fluorophenyl)-2,4-dimethyl-1H-imidazole (2.4 g, 13%) as a yellow solid. m/z: ES⁺ [M+H]⁺=297.05.

### Step 2: 1-(2-(benzyloxy)-5-fluorophenyl)-5-bromo-2,4-dimethyl-1H-imidazole

A mixture of 1-(2-(benzyloxy)-5-fluorophenyl)-2,4-dimethyl-1H-imidazole (2.4 g, 8.1 mmol, 1.0 equiv) and NBS (1.3 g, 7.3 mmol, 0.9 equiv) in DMF (24 mL) was stirred at room temperature for 1 h under nitrogen. The resulting mixture was then diluted with (200 mL) and extracted with EtOAc (3×100 mL). The combined organic layers were washed brine (2×100 mL), dried over anhydrous Na₂SO₄, and concentrated under reduced pressure. The crude product was purified using method RPFC1 (10% to 100% gradient in 20 min), resulting in 1-(2-(benzyloxy)-5-fluorophenyl)-5-bromo-2,4-dimethyl-1H-imidazole (2.4 g, 79%) as a yellow soid. m/z: ES⁺ [M+H]⁺=375.05.

### Step 3: 1-(2-(benzyloxy)-5-fluorophenyl)-2,4-dimethyl-5-(prop-1-en-2-yl)-1H-imidazole

A mixture of 1-(2-(benzyloxy)-5-fluorophenyl)-5-bromo-2,4-dimethyl-1H-imidazole (1.2 g, 3.2 mmol, 1.0 equiv), 4,4,5,5-tetramethyl-2-(prop-1-en-2-yl)-1,3,2-dioxaborolane (1.1 g, 6.4 mmol, 2.0 equiv), PdCl₂(dcypf) (483 mg, 0.64 mmol, 0.2 equiv) and K₂CO₃ (884 mg, 6.4 mmol, 0.2 equiv) in dioxane/H₂O (10:1, 13.2 mL) was stirred at 100° C. for 16 h under nitrogen. The resulting mixture was then diluted with water (50 mL) and extracted with DCM (3×50 mL). The combined organic layers were washed brine (2×100 mL), dried over anhydrous Na₂SO₄, and concentrated under reduced pressure. The crude product was purified using method RPFC1 (10% to 100% gradient in 20 min), resulting in 1-(2-(benzyloxy)-5-fluorophenyl)-2,4-dimethyl-5-(prop-1-en-2-yl)-1H-imidazole (400 mg, 37%) as a brown solid. m/z: ES⁺ [M+H]⁺=337.15.

### Step 4: 4-fluoro-2-(5-isopropyl-2,4-dimethyl-1H-imidazol-1-yl)phenol

A suspension of 1-(2-(benzyloxy)-5-fluorophenyl)-2,4-dimethyl-5-(prop-1-en-2-yl)-1H-imidazole (400 mg, 1.9 mmol, 1.0 equiv) and Pd/C (10% wt, wet, 200 mg) in MeOH (5 mL) was hydrogenated at room temperature for 16 h under hydrogen. The resulting mixture was then filtered through a Celite pad and concentrated under reduced pressure to afford 4-fluoro-2-(5-isopropyl-2,4-dimethyl-1H-imidazol-1-yl)phenol (180 mg, 61%) as a colourless solid. m/z: ES⁺ [M+H]⁺=249.05.

### 3,4-difluoro-2-(2-isopropyl-4,5-dimethyl-1H-imidazol-1-yl)phenol

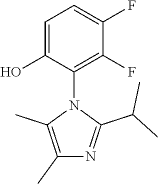 [SMILES: Cc1nc(C(C)C)n(-c2c(O)ccc(F)c2F)c1C]

### Step 1: 1-(2,3-difluoro-6-methoxyphenyl)-2-isopropyl-1H-imidazole

A solution of 2,3-difluoro-6-methoxyaniline (8.5 g, 53 mmol, 1.0 equiv), isobutyraldehyde (11.6 g, 160 mmol, 3.0 equiv), glyoxal (9.3 g, 160 mmol, 3.0 equiv), and AcONH₄ (12.35 g, 160 mmol, 3.0 equiv) in MeOH (85 mL) was stirred at 40° C. for 16 h. The reaction was diluted with water (100 mL) and extracted with EtOAc (3×100 mL). The combined organic layers were washed with brine (50 mL), dried over anhydrous Na₂SO₄, and concentrated under reduced pressure. The resulting residue was purified using method RPFC1 (70% to 80% gradient in 10 min), resulting in 1-(2,3-difluoro-6-methoxyphenyl)-2-isopropyl-1H-imidazole (6.0 g, 45%) as a yellow solid. m/z: ES⁺ [M+H]⁺=253.15.

### Step 2: 4,5-dibromo-1-(2,3-difluoro-6-methoxyphenyl)-2-isopropyl-1H-imidazole

A mixture of 1-(2,3-difluoro-6-methoxyphenyl)-2-isopropyl-1H-imidazole (2.4 g, 9.5 mmol, 1.0 equiv) and NBS (6.8 g, 38 mmol, 4.0 equiv) in DMF (25 mL) was stirred at 60° C. for 16 h under nitrogen. The resulting mixture was then diluted with water (100 mL) and extracted with EtOAc (3×100 mL). The combined organic layers were washed with brine (50 mL), dried over anhydrous Na₂SO₄, and concentrated under reduced pressure. The crude product was purified using method RPFC1 (60% to 70% gradient in 10 min), resulting in 4,5-dibromo-1-(2,3-difluoro-6-methoxyphenyl)-2-isopropyl-1H-imidazole (1.8 g, 46%) as a light-yellow solid. m/z: ES⁺ [M+H]⁺=410.95.

### Step 3: 1-(2,3-difluoro-6-methoxyphenyl)-2-isopropyl-4,5-dimethyl-1H-imidazole

A solution of 4,5-dibromo-1-(2,3-difluoro-6-methoxyphenyl)-2-isopropyl-1H-imidazole (1.6 g, 3.9 mmol, 1.0 equiv), methylboronic acid (1.4 g, 23 mmol, 6.0 equiv), Pd₂(dba)₃ (0.36 g, 0.39 mmol, 0.1 equiv), XPhos (0.28 g, 0.59 mmol, 0.15 equiv) and tBuONa (1.1 g, 12 mmol, 3.0 equiv) in dioxane (16 mL) was stirred at 100° C. for 16 h under nitrogen. The mixture was allowed to warm to room temperature, and diluted with water (100 mL), then extracted with EtOAc (3×100 mL). The combined organic layers were washed with brine (2×100 mL), dried over Na₂SO₄, and concentrated under reduced pressure. The residue was purified by silica gel column chromatography, eluting with PE/EtOAc (1:1), resulting in 1-(2,3-difluoro-6-methoxyphenyl)-2-isopropyl-4,5-dimethyl-1H-imidazole (700 mg, 64%) as a light-yellow solid. m/z: ES⁺ [M+H]⁺=281.15.

### Step 4: 3,4-difluoro-2-(2-isopropyl-4,5-dimethyl-1H-imidazol-1-yl)phenol

A solution of 1-(2,3-difluoro-6-methoxyphenyl)-2-isopropyl-4,5-dimethyl-1H-imidazole (500 mg, 1.8 mmol, 1.0 equiv) and BBr₃ (1.3 g, 5.3 mmol, 3.0 equiv) in DCM (5 mL) was stirred at 45° C. for 48 h. The resulting mixture was basified to pH-9 with sat. NaHCO₃ (aq.) and extracted with DCM (3×20 mL). The combined organic layers were dried over anhydrous Na₂SO₄, and concentrated under reduced pressure. The residue was purified using method RPFC1 (70% to 80% gradient in 10 min) to afford 3,4-difluoro-2-(2-isopropyl-4,5-dimethyl-1H-imidazol-1-yl)phenol (230 mg, 48%) as a light-yellow solid. m/z: ES⁺ [M+H]⁺=267.10.

### 2-(2,4-diisopropyl-1H-imidazol-1-yl)-4-fluorophenol

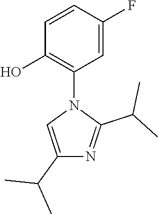 [SMILES: CC(C)c1cn(-c2cc(F)ccc2O)c(C(C)C)n1]

### Step 1: 4,5-dibromo-1-(5-fluoro-2-methoxyphenyl)-2-isopropyl-1H-imidazole

A mixture of 1-(5-fluoro-2-methoxyphenyl)-2-isopropyl-1H-imidazole (cAS 2169922-00-1) (3.5 g, 15 mmol, 1.0 equiv) and NBS (10.6 g, 60 mmol, 4.0 equiv) in DMF (30 mL) was stirred at room temperature for 5 h under nitrogen. The resulting mixture was then diluted with water (100 mL) and extracted with EtOAc (3×100 mL). The combined organic layers were washed brine (100 mL), dried over anhydrous Na₂SO₄, and concentrated under reduced pressure. The crude product was purified using method RPFC1 (70% to 80% gradient in 10 min), resulting in 4,5-dibromo-1-(5-fluoro-2-methoxyphenyl)-2-isopropyl-1H-imidazole (3.0 g, 51%) as alight-yellow oil. m/z: ES⁺ [M+H]⁺=392.95.

### Step 2: 4-bromo-1-(5-fluoro-2-methoxyphenyl)-2-isopropyl-1H-imidazole

nBuL₁ (2.5 M in THF, 2.4 mL, 5.9 mmol, 1.0 equiv) was added to a stirred solution of 4,5-dibromo-1-(5-fluoro-2-methoxyphenyl)-2-isopropyl-1H-imidazole (2.3 g, 5.9 mmol, 1.0 equiv) in THF (20 mL) dropwise at −75° C. under nitrogen. The resulting mixture was stirred at 0° C. for 2 h under nitrogen, and then was quenched by the addition of sat. NH₄Cl (aq.) at 0° C. The mixture was extracted with EtOAc (3×100 mL). The combined organic layers were washed with brine (100 mL), dried over anhydrous Na₂SO₄, and concentrated under reduced pressure. The residue was purified by silica gel column chromatography, eluting with PE/EtOAc (1:1) to afford 4-bromo-1-(5-fluoro-2-methoxyphenyl)-2-isopropyl-1H-imidazole (900 mg, 49%) as a light-yellow oil. m/z: ES⁺ [M+H]⁺=326.10.

### Step 3: 1-(5-fluoro-2-methoxyphenyl)-2-isopropyl-4-(prop-1-en-2-yl)-1H-imidazole

A solution of 4-bromo-1-(5-fluoro-2-methoxyphenyl)-2-isopropyl-1H-imidazole (900 mg, 2.9 mmol, 1.0 equiv), 4,4,5,5-tetramethyl-2-(prop-1-en-2-yl)-1,3,2-dioxaborolane (966 mg, 5.7 mmol, 2.0 equiv), Pd(dppf)Cl₂CH₂Cl₂ (234 mg, 029 mmol, 0.1 equiv), and K₂CO₃ (1.19 g, 8.6 mmol, 3.0 equiv) in dioxane/water (4:1, 10 mL) was stirred at 100° C. for 4 h under nitrogen. The mixture was allowed to warm to room temperature, and diluted with water (10 mL), then extracted with EtOAc (3×10 mL). The combined organic layers were washed with brine (10 mL), dried over Na₂SO₄, and concentrated under reduced pressure. The residue was purified by silica gel column chromatography, eluting with PE/EtOAc (1:1), resulting in 1-(5-fluoro-2-methoxyphenyl)-2-isopropyl-4-(prop-1-en-2-yl)-1H-imidazole (450 mg, 57%) as a light-yellow oil. m/z: ES⁺ [M+H]⁺=275.20.

### Step 4: 1-(5-fluoro-2-methoxyphenyl)-2,4-diisopropyl-1H-imidazole

A suspension of 1-(5-fluoro-2-methoxyphenyl)-2-isopropyl-4-(prop-1-en-2-yl)-1H-imidazole (450 mg, 1.6 mmol, 1.0 equiv) and Pd/C (10% wt, wet, 50 mg) in MeOH (5 mL) was hydrogenated at room temperature for 2 h under hydrogen. The resulting mixture was then filtered through a Celite pad and the solids washed with MeOH (5 mL). The filtrate was concentrated under reduced pressure to afford 1-(5-fluoro-2-methoxyphenyl)-2,4-diisopropyl-1H-imidazole (350 mg, 77%) as a light-yellow oil. m/z: ES⁺ [M+H]⁺=277.15.

### Step 5: 2-(2,4-diisopropyl-1H-imidazol-1-yl)-4-fluorophenol

A solution of 1-(5-fluoro-2-methoxyphenyl)-2,4-diisopropyl-1H-imidazole (350 mg, 1.3 mmol, 1.0 equiv) and BBr₃ (951 mg, 3.8 mmol, 3.0 equiv) in DCM (3 mL) was stirred at room temperature for 2 h under nitrogen. The resulting mixture was quenched with sat. NH₄Cl (aq.) at 0° C. and extracted with DCM (3×10 mL). The combined organic layers were dried over anhydrous Na₂SO₄, and concentrated under reduced pressure. The residue was was purified using method RPFC1 (50% to 60% gradient in 10 min) to afford 2-(2,4-diisopropyl-1H-imidazol-1-yl)-4-fluorophenol (250 mg, 75%) as a light-yellow oil. m/z: ES⁺ [M+H]⁺=263.20.

### rac-2-(1-(dimethylamino)-2,2,2-trifluoroethyl)-4-fluorophenol

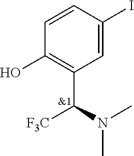 [SMILES: CN(C)[C@H](c1cc(I)ccc1O)C(F)(F)F]

### Step 1: rac-2-methyl-N-(2,2,2-trifluoro-1-(5-fluoro-2-methoxyphenyl)ethyl)propane-2-sulfinamide

To a stirred solution of rac-(E)-N-(5-fluoro-2-methoxybenzylidene)-2-methylpropane-2-sulfinamide (CAS 2410738-83-7/2374199-40-1) (11 g, 43 mmol, 1.0 equiv) and TBAE (13 g, 43 mmol, 1.0 equiv) in DMF (110 mL) was added TMSOTf (15.2 g, 107 mmol, 2.5 equiv) at 0° C. The resulting mixture was stirred at 0° C. for 1 h under nitrogen and then quenched with sat. NH₄Cl (aq.) (200 mL) and extracted with EtOAc (2×200 mL). The combined organic layers were washed with brine (2×200 mL), dried over anhydrous Na₂SO₄, and concentrated under reduced pressure. The residue was purified by RPFC1 (10% to 100% gradient in 20 min) to afford rac-2-methyl-N-(2,2,2-trifluoro-1-(5-fluoro-2-methoxyphenyl)ethyl)propane-2-sulfinamide (11 g, 79%) as a yellow solid. m/z: ES⁺ [M+H]⁺=328.00.

### Step 2: rac-2,2,2-trifluoro-1-(5-fluoro-2-methoxyphenyl)ethan-1-amine

A solution of rac-2-methyl-N-(2,2,2-trifluoro-1-(5-fluoro-2-methoxyphenyl)ethyl)propane-2-sulfinamide (11 g, 34 mmol, 1.0 equiv) and HCl in 1,4-dioxane (4.0 M, 100 mL) in MeOH (100 mL) was stirred at room temperature for 2 h. The resulting mixture was concentrated under reduced pressure and triturated with EtOAc (50 mL). The precipitate was collected by filtration and washed with EtOAc (3×30 mL), resulting in rac-2,2,2-trifluoro-1-(5-fluoro-2-methoxyphenyl)ethan-1-amine hydrochloride (7.0 g, 88%) as a colourless solid. m/z: ES⁺ [M+H]⁺=224.10.

### Step 3: rac-2,2,2-trifluoro-1-(5-fluoro-2-methoxyphenyl)-N,N-dimethylethan-1-amine

The reductive amination of rac-2,2,2-trifluoro-1-(5-fluoro-2-methoxyphenyl)ethan-1-amine hydrochloride (1.2 g, 4.6 mmol, 1.0 equiv) was performed according to general procedure H1. The residue was purified by RPFC1 (10% to 100% gradient in 20 min) to afford rac-2,2,2-trifluoro-1-(5-fluoro-2-methoxyphenyl)-N,N-dimethylethan-1-amine (550 mg, 47%) as a brown oil. m/z: ES⁺ [M+H]⁺=252.05.

### Step 4: rac-2-(1-(dimethylamino)-2,2,2-trifluoroethyl)-4-fluorophenol

A solution of rac-2,2,2-trifluoro-1-(5-fluoro-2-methoxyphenyl)-N,N-dimethylethan-1-amine (250 mg, 1.0 mmol, 1.0 equiv) and BBr₃ (1 M in DCM, 4.0 mL, 4.0 mmol, 4.0 equiv) in DCM (1 mL) was stirred at room temperature for 16 h under nitrogen. The resulting mixture was quenched with MeOH at 0° C. and concentrated under reduced pressure to afford rac-2-(1-(dimethylamino)-2,2,2-trifluoroethyl)-4-fluorophenol (200 mg, 85%) as a brown oil. m/z: ES⁺ [M+H]⁺=238.00.

### 2-(2-(tert-butyl)-4,5-dimethyl-1H-imidazol-1-yl)-4-fluorophenol

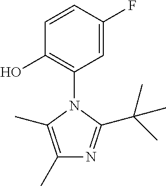 [SMILES: Cc1nc(C(C)(C)C)n(-c2cc(F)ccc2O)c1C]

### Step 1: 4-bromo-2-(tert-butyl)-1-(5-fluoro-2-methoxyphenyl)-5-methyl-1H-imidazole

A mixture of 2-(tert-butyl)-1-(5-fluoro-2-methoxyphenyl)-5-methyl-1H-imidazole (500 mg, 1.9 mmol, 1.0 equiv) and NBS (373 mg, 2.1 mmol, 1.1 equiv) in MeCN (6 mL) was stirred at room temperature for 21 h under nitrogen. The resulting mixture was then diluted with water (10 mL) and extracted with DCM (3×10 mL). The combined organic layers were dried over anhydrous Na₂SO₄, and concentrated under reduced pressure, resulting in 4-bromo-2-(tert-butyl)-1-(5-fluoro-2-methoxyphenyl)-5-methyl-1H-imidazole (500 mg, 77%) as a yellow solid. m/z: ES⁺ [M+H]⁺=341.06.

### Step 2: 2-(tert-butyl)-1-(5-fluoro-2-methoxyphenyl)-4,5-dimethyl-1H-imidazole

A mixture of 4-bromo-2-(tert-butyl)-1-(5-fluoro-2-methoxyphenyl)-5-methyl-1H-imidazole (430 mg, 1.3 mmol, 1.0 equiv), methylboronic acid (226 g, 3.8 mmol, 3.0 equiv), Pd₂(dba)₃ (115 g, 0.13 mmol, 0.1 equiv), XPhos (60 mg, 0.13 mmol, 0.1 equiv) and tBuONa (242 mg, 2.5 mmol, 2.0 equiv) in dioxane (5 mL) was stirred at 100° C. for 5 h under nitrogen. The resulting mixture was then diluted with water (10 mL) and extracted with EtOAc (2×10 mL). The combined organic layers were dried over anhydrous Na₂SO₄, and concentrated under reduced pressure. The crude product was purified using method RPFC1 (10% to 100% gradient in 20 min), resulting in 2-(tert-butyl)-1-(5-fluoro-2-methoxyphenyl)-4,5-dimethyl-1H-imidazole (230 mg, 66%) as a yellow solid. m/z: ES⁺ [M+H]⁺=277.16.

### Step 4: 2-(2-(tert-butyl)-4,5-dimethyl-1H-imidazol-1-yl)-4-fluorophenol

A solution of 2-(tert-butyl)-1-(5-fluoro-2-methoxyphenyl)-4,5-dimethyl-1H-imidazole (200 mg, 0.7 mmol, 1.0 equiv) in aq. HBr (48% in water, 2 mL) was stirred at 100° C. for 16 h under nitrogen. The resulting mixture was diluted with water (10 mL) and extracted with EtOAc (2×10 mL). The combined organic layers were dried over anhydrous Na₂SO₄, and concentrated under reduced pressure. The crude product was purified using method RPFC1 (10% to 100% gradient in 10 min), resulting in 2-(2-(tert-butyl)-4,5-dimethyl-1H-imidazol-1-yl)-4-fluorophenol (100 mg, 53%) as a colourless solid. m/z: ES⁺ [M+H]⁺=263.15.

### 2-(4-ethyl-2-isopropyl-5-methyl-1H-imidazol-1-yl)-4-fluorophenol

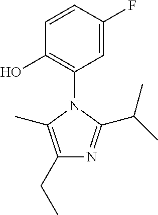 [SMILES: CCc1nc(C(C)C)n(-c2cc(F)ccc2O)c1C]

### Step 1: 1-(2-(benzyloxy)-5-fluorophenyl)-2-isopropyl-5-methyl-1H-imidazole

A solution of N-(prop-2-yn-1-yl)isobutyramide (1.5 g, 12 mmol, 1.0 equiv), 2-(benzyloxy)-5-fluoroaniline (2.6 g, 12 mmol, 1.0 equiv) and Zn(OTf)₂ (1.3 g, 3.6 mmol, 0.3 equiv) in toluene (15 mL) was stirred at 90° C. for 16 h under nitrogen. The resulting mixture was diluted with water (50 mL) and extracted with EtOAc (2×50 mL). The combined organic layers were washed with water (2×50 mL), dried over anhydrous Na₂SO₄, and concentrated under reduced pressure. The residue was purified using method RPFC1 (50% to 60% gradient in 10 min), resulting in 1-(2-(benzyloxy)-5-fluorophenyl)-2-isopropyl-5-methyl-1H-imidazole (900 mg, 23%) as a yellow oil. m/z: ES⁺ [M+H]⁺=325.15.

### Step 2: 1-(2-(benzyloxy)-5-fluorophenyl)-4-bromo-2-isopropyl-5-methyl-1H-imidazole

A mixture of 1-(2-(benzyloxy)-5-fluorophenyl)-2-isopropyl-5-methyl-1H-imidazole (900 mg, 2.8 mmol, 1.0 equiv) and NBS (988 mg, 5.5 mmol, 2.0 equiv) in DMF (9 mL) was stirred at room temperature for 5 h under nitrogen. The resulting mixture was then diluted with water (50 mL) and extracted with EtOAc (2×50 mL). The combined organic layers were dried over anhydrous Na₂SO₄, and concentrated under reduced pressure. The residue was purified using method RPFC1 (60% to 70% gradient in 10 min), resulting in 1-(2-(benzyloxy)-5-fluorophenyl)-4-bromo-2-isopropyl-5-methyl-1H-imidazole (400 mg, 36%) as a yellow oil. m/z: ES⁺ [M+H]⁺=403.05.

### Step 3: 1-(2-(benzyloxy)-5-fluorophenyl)-2-isopropyl-5-methyl-4-vinyl-1H-imidazole

A mixture of 1-(2-(benzyloxy)-5-fluorophenyl)-4-bromo-2-isopropyl-5-methyl-1H-imidazole (430 mg, 0.99 mmol, 1.0 equiv), 2-ethenyl-4,4,5,5-tetramethyl-1,3,2-dioxaborolane (183 g, 1.2 mmol, 1.2 equiv), Pd₂(dba)₃ (182 mg, 0.20 mmol, 0.2 equiv), XPhos (71 mg, 0.15 mmol, 0.15 equiv) and Cs₂CO₃ (970 mg, 3.0 mmol, 3.0 equiv) in dioxane (5 mL) was stirred at 100° C. for 16 h under nitrogen. The resulting mixture was then diluted with water (10 mL) and extracted with EtOAc (2×10 mL). The residue was purified using method RPFC1 (10% to 50% gradient in 10 min), resulting in 1-(2-(benzyloxy)-5-fluorophenyl)-2-isopropyl-5-methyl-4-vinyl-1H-imidazole (300 mg, 86%) as a yellow solid. m/z: ES⁺ [M+H]⁺=351.20.

### Step 4: 2-(4-ethyl-2-isopropyl-5-methyl-1H-imidazol-1-yl)-4-fluorophenol

A suspension of 1-(2-(benzyloxy)-5-fluorophenyl)-2-isopropyl-5-methyl-4-vinyl-1H-imidazole (300 mg, 0.86 mmol, 1.0 equiv) and Pd(OH)₂/C (10% wt, dry, 120 mg) in EtOAc (3 mL) was hydrogenated at room temperature for 2 h under hydrogen. The resulting mixture was then filtered through Celite and concentrated under reduced pressure. The residue was purified using method RPFC1 (50% to 60% gradient in 10 min), to afford 2-(4-ethyl-2-isopropyl-5-methyl-1H-imidazol-1-yl)-4-fluorophenol (200 mg, 89%) as a yellow oil. m/z: ES⁺ [M+H]⁺=263.05.

### 2-(4-ethyl-5-isopropyl-2-methyl-1H-imidazol-1-yl)-4-fluorophenol

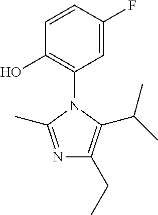 [SMILES: CCc1nc(C)n(-c2cc(F)ccc2O)c1C(C)C]

### Step 1: 1-(5-fluoro-2-methoxyphenyl)-2-methyl-1H-imidazole

A solution of 5-fluoro-2-methoxyphenylboronic acid (8.0 g, 47 mmol, 1.0 equiv), Cu(OAc)₂ (4.3 g, 24 mmol, 0.5 equiv), 2-methylimidazole (7.7 g, 94 mmol, 2.0 equiv), and NEt₃ (14.3 g, 141 mmol, 3.0 equiv) in MeOH (150 mL) was stirred at room temperature for 16 h. The resulting mixture was filtered, the filter cake was washed with EtOAc (2×50 mL), and the filtrate was concentrated under reduced pressure. The residue was purified by silica gel column chromatography, eluting with PE/EtOAc (1:1), to give 1-(5-fluoro-2-methoxyphenyl)-2-methyl-1H-imidazole (4.0 g, 41%) as a light-yellow oil. m/z: ES⁺ [M+H]⁺=207.09.

### Step 2: 5-bromo-1-(5-fluoro-2-methoxyphenyl)-2-methyl-1H-imidazole

A mixture of 1-(5-fluoro-2-methoxyphenyl)-2-methyl-1H-imidazole (3.5 g, 17 mmol, 1.0 equiv) and NBS (3.3 g, 19 mmol, 1.1 equiv) in DMF (60 mL) was stirred at room temperature for 2 h under nitrogen. The resulting mixture was then diluted with water (50 mL) and extracted with EtOAc (2×50 mL). The combined organic layers were washed with water (2×50 mL), dried over anhydrous Na₂SO₄, and concentrated under reduced pressure. The residue was purified using method RPFC1 (30% to 90% gradient in 25 min), resulting in 5-bromo-1-(5-fluoro-2-methoxyphenyl)-2-methyl-1H-imidazole (3.0 g, 62%) as a yellow oil. m/z: ES⁺ [M+H]⁺=285.12.

### Step 3: 1-(5-fluoro-2-methoxyphenyl)-2-methyl-5-(prop-1-en-2-yl)-1H-imidazole

A mixture of 5-bromo-1-(5-fluoro-2-methoxyphenyl)-2-methyl-1H-imidazole (2.2 g, 7.7 mmol, 1.0 equiv), 4,4,5,5-tetramethyl-2-(prop-1-en-2-yl)-1,3,2-dioxaborolane (2.6 g, 15 mmol, 2.0 equiv), Pd(dppf)Cl₂CH₂Cl₂ (1.3 g, 1.5 mmol, 0.2 equiv) and K₂CO₃ (2.1 g, 15 mmol, 2.0 equiv) in dioxane (20 mL) and water (4 mL) was stirred at 100° C. for 16 h under nitrogen. The resulting mixture was filtered, the filter cake was washed with EtOAc (2×50 mL), and the filtrate was concentrated under reduced pressure. The resulting residue was purified using method RPFC4 (40% to 90% gradient in 10 min) to afford 1-(5-fluoro-2-methoxyphenyl)-2-methyl-5-(prop-1-en-2-yl)-1H-imidazole (1.4 g, 74%) as a light-yellow solid. m/z: ES⁺ [M+H]⁺=247.29.

### Step 4: 1-(5-fluoro-2-methoxyphenyl)-5-isopropyl-2-methyl-1H-imidazole

A mixture of 1-(5-fluoro-2-methoxyphenyl)-2-methyl-5-(prop-1-en-2-yl)-1H-imidazole (1.35 g, 5.5 mmol, 1.0 equiv) and Pd/C (2.3 g, 10% wt, wet) in MeOH (20 mL) was stirred at room temperature for 2 h under hydrogen. The resulting mixture was filtered and the filter cake was washed with MeOH (20 mL). The filtrate was concentrated under reduced pressure to afford 1-(5-fluoro-2-methoxyphenyl)-5-isopropyl-2-methyl-1H-imidazole (1.2 g, 88%) as a yellow solid. m/z: ES⁺ [M+H]⁺=249.30.

### Step 5: 4-bromo-1-(5-fluoro-2-methoxyphenyl)-5-isopropyl-2-methyl-1H-imidazole

A mixture of 1-(5-fluoro-2-methoxyphenyl)-5-isopropyl-2-methyl-1H-imidazole (1.2 g, 4.8 mmol, 1.0 equiv) and NBS (950 mg, 5.3 mmol, 1.1 equiv) in DMF (209 mL) was stirred at room temperature for 2 h under nitrogen. The resulting mixture was then diluted with water (50 mL) and extracted with EtOAc (2×50 mL). The combined organic layers were washed with water (2×50 mL), dried over anhydrous Na₂SO₄, and concentrated under reduced pressure. The residue was purified method RPFC1 (40% to 90% gradient in 10 min), resulting in 4-bromo-1-(5-fluoro-2-methoxyphenyl)-5-isopropyl-2-methyl-1H-imidazole (1.2 g, 76%) as a brown solid. m/z: ES⁺ [M+H]⁺=327.20.

### Step 6: 1-(5-fluoro-2-methoxyphenyl)-5-isopropyl-2-methyl-4-vinyl-1H-imidazole

A mixture of 4-bromo-1-(5-fluoro-2-methoxyphenyl)-5-isopropyl-2-methyl-1H-imidazole (1.0 g, 3.1 mmol, 1.0 equiv), 2-ethenyl-4,4,5,5-tetramethyl-1,3,2-dioxaborolane (1.44 g, 6.1 mmol, 2.0 equiv), Pd₂(dba)₃ (560 mg, 0.61 mmol, 0.2 equiv), XPhos (291 mg, 0.61 mmol, 0.2 equiv) and tBuONa (734 mg, 7.6 mmol, 2.5 equiv) in dioxane (20 mL) was stirred at 100° C. for 16 h under nitrogen. The resulting mixture was filtered and the filter cake was washed with EtOAc (2×50 mL). The filtrate was concentrated under reduced pressure, and the resulting residue was purified using method RPFC1 (40% to 90% gradient in 10 min), resulting in 1-(5-fluoro-2-methoxyphenyl)-5-isopropyl-2-methyl-4-vinyl-1H-imidazole (700 mg, 83%) as alight-yellow solid. m/z: ES⁺ [M+H]⁺=275.15.

### Step 7: 4-ethyl-1-(5-fluoro-2-methoxyphenyl)-5-isopropyl-2-methyl-1H-imidazole

A mixture of 1-(5-fluoro-2-methoxyphenyl)-5-isopropyl-2-methyl-4-vinyl-1H-imidazole (350 mg, 1.3 mmol, 1.0 equiv) and Pd/C (543 mg, 10% wt, wet) in MeOH (5 mL) was stirred at room temperature for 2 h under hydrogen. The resulting mixture was filtered and the filter cake was washed with MeOH (5 mL). The filtrate was concentrated under reduced pressure to afford 4-ethyl-1-(5-fluoro-2-methoxyphenyl)-5-isopropyl-2-methyl-1H-imidazole (250 mg, 71%) as abrown solid. m/z: ES⁺ [M+H]⁺=277.16.

### Step 8: 2-(4-ethyl-5-isopropyl-2-methyl-1H-imidazol-1-yl)-4-fluorophenol

A solution of 4-ethyl-1-(5-fluoro-2-methoxyphenyl)-5-isopropyl-2-methyl-1H-imidazole (280 mg, 1.0 mmol, 1.0 equiv) and BBr₃ (1.0 g, 4.0 mmol, 4.0 equiv) in DCM (10 mL) was stirred at room temperature for 16 h under nitrogen. The resulting mixture was quenched with MeOH (2 mL) at 0° C. and concentrated under reduced pressure to afford 2-(4-ethyl-5-isopropyl-2-methyl-1H-imidazol-1-yl)-4-fluorophenol (150 mg, 56%) as a brown solid. m/z: ES⁺ [M+H]⁺=263.33.

### 4-fluoro-2-(2-isopropyl-5,6-dihydrocyclopenta[d]imidazol-1(4H)-yl)phenol

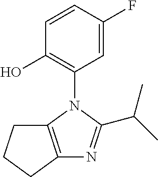 [SMILES: CC(C)c1nc2c(n1-c1cc(F)ccc1O)CCC2]

### Step 1: 1-(2-(benzyloxy)-5-fluorophenyl)-1,4,5,6-tetrahydrocyclopenta[d]imidazole

A solution of (2-(benzyloxy)-5-fluorophenyl)boronic acid (1.1 g, 4.6 mmol, 1.0 equiv), Cu(OAc)₂ (168 mg, 0.92 mmol, 0.2 equiv), 1,4,5,6-tetrahydrocyclopenta[d]imidazole (500 mg, 4.6 mmol, 1.0 equiv), and NEt₃ (1.4 g, 14 mmol, 3.0 equiv) in MeOH (10 mL) was stirred at room temperature for 16 h. The resulting mixture was poured into water (30 mL) and extracted with DCM (3×10 mL). The combined organic layers were washed with brine (3×10 mL), dried over anhydrous Na₂SO₄, and concentrated under reduced pressure. The residue was purified using method RPFC1 (10% to 100% gradient in 10 min), resulting in 1-(2-(benzyloxy)-5-fluorophenyl)-1,4,5,6-tetrahydrocyclopenta[d]imidazole (1.3 g, 91%) as a brown oil. m/z: ES⁺ [M+H]⁺=309.00.

### Step 2: 1-(2-(benzyloxy)-5-fluorophenyl)-2-bromo-1,4,5,6-tetrahydrocyclopenta[d]imidazole

A mixture of 1-(2-(benzyloxy)-5-fluorophenyl)-1,4,5,6-tetrahydrocyclopenta[d]imidazole (500 mg, 1.6 mmol, 1.0 equiv) and NBS (289 mg, 1.6 mmol, 1.0 equiv) in DMF (5 mL) was stirred at room temperature for 2 h under nitrogen. The resulting mixture was then diluted with water (30 mL) and extracted with DCM (3×10 mL). The combined organic layers were washed with brine (3×10 mL), dried over anhydrous Na₂SO₄, and concentrated under reduced pressure. The residue was purified using method RPFC1 (10% to 100% gradient in 20 min), resulting in 1-(2-(benzyloxy)-5-fluorophenyl)-2-bromo-1,4,5,6-tetrahydrocyclopenta[d]imidazole (200 mg, 32%) as a brown oil. m/z: ES⁺ [M+H]⁺=388.85.

### Step 3: 1-(2-(benzyloxy)-5-fluorophenyl)-2-(prop-1-en-2-yl)-1,4,5,6-tetrahydrocyclopenta[d]imidazole

A mixture of 1-(2-(benzyloxy)-5-fluorophenyl)-2-bromo-1,4,5,6-tetrahydrocyclopenta[d]imidazole (210 mg, 0.54 mmol, 1.0 equiv), 4,4,5,5-tetramethyl-2-(prop-1-en-2-yl)-1,3,2-dioxaborolane (137 g, 0.81 mmol, 1.5 equiv), Pd(dppf)Cl₂CH₂Cl₂ (66 mg, 0.08 mmol, 0.15 equiv) and K₂CO₃ (225 mg, 1.6 mmol, 3.0 equiv) in dioxane (3 mL) and water (1 mL) was stirred at 100° C. for 2 h under nitrogen. The resulting mixture was extracted with DCM (3×10 mL). The combined organic layers were washed with brine (3×10 mL), dried over anhydrous Na₂SO₄, and concentrated under reduced pressure. The residue was purified using method RPFC1 (10% to 100% gradient in 10 min) to afford 1-(2-(benzyloxy)-5-fluorophenyl)-2-(prop-1-en-2-yl)-1,4,5,6-tetrahydrocyclopenta[d]imidazole (100 mg, 53%) as a brown oil. m/z: ES⁺ [M+H]⁺=349.05.

### Step 4: 4-fluoro-2-(2-isopropyl-5,6-dihydrocyclopenta[d]imidazol-1(4H)-yl)phenol

A mixture of 1-(2-(benzyloxy)-5-fluorophenyl)-2-(prop-1-en-2-yl)-1,4,5,6-tetrahydrocyclopenta[d]imidazole (100 mg, 0.23 mmol, 1.0 equiv) and Pd/C (50 mg, 0.47 mmol, 1.6 equiv) in MeOH (2 mL) was stirred at room temperature for 16 h under hydrogen. The resulting mixture was filtered and the filter cake was washed with MeOH (3×10 mL). The filtrate was concentrated under reduced pressure to afford 4-fluoro-2-(2-isopropyl-5,6-dihydrocyclopenta[d]imidazol-1(4H)-yl)phenol (70 mg, 64%) as a brown solid. m/z: ES⁺ [M+H]⁺=261.00.

### 2-(5-ethyl-2-isopropyl-4-methyl-1H-imidazol-1-yl)-4-fluorophenol

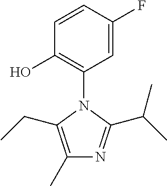 [SMILES: CCc1c(C)nc(C(C)C)n1-c1cc(F)ccc1O]

### Step 1: 5-bromo-1-(5-fluoro-2-methoxyphenyl)-2-isopropyl-1H-imidazole

A mixture of 5-bromo-1-(5-fluoro-2-methoxyphenyl)-2-isopropyl-1H-imidazole (5.5 g, 23 mmol, 1.0 equiv) and NBS (4.2 g, 23 mmol, 1.0 equiv) in DMF (40 mL) was stirred at room temperature for 2 h under nitrogen. The resulting mixture was then diluted with water (50 mL) and extracted with EtOAc (2×50 mL). The combined organic layers were washed with water (2×50 mL), dried over anhydrous Na₂SO₄, and concentrated under reduced pressure. The residue was purified using method RPFC1 (10% to 100% gradient in 20 min), resulting in 5-bromo-1-(5-fluoro-2-methoxyphenyl)-2-isopropyl-1H-imidazole (4.5 g, 61%) as a light-yellow oil. m/z: ES⁺ [M+H]⁺=313.15.

### Step 2: 1-(5-fluoro-2-methoxyphenyl)-2-isopropyl-5-vinyl-1H-imidazole

A mixture of 5-bromo-1-(5-fluoro-2-methoxyphenyl)-2-isopropyl-1H-imidazole (2.2 g, 7.0 mmol, 1.0 equiv), 4,4,5,5-tetramethyl-2-vinyl-1,3,2-dioxaborolane (2.2 g, 14 mmol, 2.0 equiv), Pd(dppf)Cl₂CH₂Cl₂ (1.15 g, 1.4 mmol, 0.2 equiv) and K₂CO₃ (2.9 g, 21 mmol, 3.0 equiv) in dioxane (15 mL) and water (3 mL) was stirred at 100° C. for 16 h under nitrogen. The resulting mixture was extracted with EtOAc (2×50 mL). The combined organic layers were washed with water (2×50 mL), dried over anhydrous Na₂SO₄, and concentrated under reduced pressure. The residue was purified using method RPFC1 (10% to 100% gradient in 20 min) to afford 1-(5-fluoro-2-methoxyphenyl)-2-isopropyl-5-vinyl-1H-imidazole (1.4 g, 77%) as a light-yellow oil. m/z: ES⁺ [M+H]⁺=261.05.

### Step 3: 5-ethyl-1-(5-fluoro-2-methoxyphenyl)-2-isopropyl-1H-imidazole

A mixture of 1-(5-fluoro-2-methoxyphenyl)-2-isopropyl-5-vinyl-1H-imidazole (1.4 g, 5.4 mmol, 1.0 equiv) and Pd/C (10% wt, 0.57 g, wet) in MeOH (30 mL) was stirred at room temperature for 2 h under hydrogen. The resulting mixture was filtered and the filtrate was concentrated under reduced pressure to afford 5-ethyl-1-(5-fluoro-2-methoxyphenyl)-2-isopropyl-1H-imidazole (1.0 g, 81%) as a light-yellow oil. m/z: ES⁺ [M+H]⁺=263.05.

### Step 4: 4-bromo-5-ethyl-1-(5-fluoro-2-methoxyphenyl)-2-isopropyl-1H-imidazole

A mixture of 5-ethyl-1-(5-fluoro-2-methoxyphenyl)-2-isopropyl-1H-imidazole (1.0 g, 3.8 mmol, 1.0 equiv) and NBS (678 mg, 3.8 mmol, 1.0 equiv) in DMF (10 mL) was stirred at room temperature for 2 h under nitrogen. The resulting mixture was then diluted with water (50 mL) and extracted with EtOAc (2×50 mL). The combined organic layers were washed with water (2×50 mL), dried over anhydrous Na₂SO₄, and concentrated under reduced pressure. The residue was purified using method RPFC1 (10% to 100% gradient in 20 min), resulting in 4-bromo-5-ethyl-1-(5-fluoro-2-methoxyphenyl)-2-isopropyl-1H-imidazole (900 mg, 69%) as a brown solid. m/z: ES⁺ [M+H]⁺=341.15.

### Step 5: 5-ethyl-1-(5-fluoro-2-methoxyphenyl)-2-isopropyl-4-methyl-1H-imidazole

A mixture of 4-bromo-5-ethyl-1-(5-fluoro-2-methoxyphenyl)-2-isopropyl-1H-imidazole (1.0 g, 2.9 mmol, 1.0 equiv), methylboronic acid (701 mg, 12 mmol, 4.0 equiv), Pd₂(dba)₃ (536 mg, 0.59 mmol, 0.2 equiv), XPhos (279 mg, 0.59 mmol, 0.2 equiv) and tBuONa (845 mg, 8.8 mmol, 2.0 equiv) in dioxane (20 mL) was stirred at 100° C. for 16 h under nitrogen. The resulting mixture was then diluted with water (50 mL) and extracted with EtOAc (2×50 mL). The combined organic layers were dried over anhydrous Na₂SO₄ and concentrated under reduced pressure. The crude product was purified using method RPFC1 (10% to 100% gradient in 20 min), resulting in 5-ethyl-1-(5-fluoro-2-methoxyphenyl)-2-isopropyl-4-methyl-1H-imidazole (940 mg, 64%) as a yellow solid. m/z: ES⁺ [M+H]⁺=277.15.

### Step 6: 2-(5-ethyl-2-isopropyl-4-methyl-1H-imidazol-1-yl)-4-fluorophenol

A solution of 5-ethyl-1-(5-fluoro-2-methoxyphenyl)-2-isopropyl-4-methyl-1H-imidazole (940 mg, 3.4 mmol, 1.0 equiv) and BBr₃ (8.5 g, 34 mmol, 10 equiv) in DCM (2 mL) was stirred at 40° C. for 16 h under nitrogen. The resulting mixture was quenched with MeOH at 0° C., concentrated under reduced pressure and the crude product was purified using method RPFC1 (10% to 100% gradient in 20 min), to afford 2-(5-ethyl-2-isopropyl-4-methyl-1H-imidazol-1-yl)-4-fluorophenol (300 mg, 34%) as a brown oil. m/z: ES⁺ [M+H]⁺=263.10.

### 2-(2,5-diisopropyl-4-methyl-1H-imidazol-1-yl)-4-fluorophenol

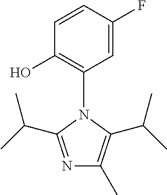 [SMILES: Cc1nc(C(C)C)n(-c2cc(F)ccc2O)c1C(C)C]

### Step 1: 1-(5-fluoro-2-methoxyphenyl)-2-isopropyl-5-(prop-1-en-2-yl)-1H-imidazole

A solution of 5-bromo-1-(5-fluoro-2-methoxyphenyl)-2-isopropyl-1H-imidazole (2.5 g, 8.0 mmol, 1.0 equiv), 4,4,5,5-tetramethyl-2-(prop-1-en-2-yl)-1,3,2-dioxaborolane (1.6 g, 9.6 mmol, 1.2 equiv), Pd(dppf)Cl₂CH₂Cl₂ (652 mg, 0.80 mmol, 0.1 equiv), and K₂CO₃ (3.31 g, 24 mmol, 3.0 equiv) in dioxane/water (4:1, 25 mL) was stirred at 100° C. for 12 h under nitrogen. The mixture was allowed to warm to room temperature, diluted with water (50 mL), then extracted with EtOAc (2×50 mL). The combined organic layers were washed with water (2×50 mL), dried over Na₂SO₄, and concentrated under reduced pressure. The residue was purified using method RPFC1 (10% to 100% gradient in 30 min), resulting in 1-(5-fluoro-2-methoxyphenyl)-2-isopropyl-5-(prop-1-en-2-yl)-1H-imidazole (900 mg, 41%) as a light-yellow oil. m/z: ES⁺ [M+H]⁺=275.10.

### Step 2: 1-(5-fluoro-2-methoxyphenyl)-2,5-diisopropyl-1H-imidazole

A suspension of 1-(5-fluoro-2-methoxyphenyl)-2-isopropyl-5-(prop-1-en-2-yl)-1H-imidazole (800 mg, 2.9 mmol, 1.0 equiv) and Pd/C (10% wt, wet, 200 mg) in MeOH (1 mL) was hydrogenated at room temperature for 4 h under hydrogen. The resulting mixture was then filtered through a Celite pad and the solids washed with MeOH (5×5 mL). The filtrate was concentrated under reduced pressure to afford crude 1-(5-fluoro-2-methoxyphenyl)-2,5-diisopropyl-1H-imidazole (800 mg, quant.), which was used directly in the next step. m/z: ES⁺ [M+H]⁺=277.00.

### Step 3: 4-bromo-1-(5-fluoro-2-methoxyphenyl)-2,5-diisopropyl-1H-imidazole

A mixture of 1-(5-fluoro-2-methoxyphenyl)-2,5-diisopropyl-1H-imidazole (800 mg, 2.9 mmol, 1.0 equiv) and NBS (571 mg, 3.2 mmol, 1.1 equiv) in DMF (1 mL) was stirred at room temperature for 4 h under nitrogen. The resulting mixture was then quenched by the addition of sat. NaHCO₃ (aq.) (5 mL) and extracted with EtOAc (3×5 mL). The combined organic layers were washed brine (5 mL), dried over anhydrous Na₂SO₄, and concentrated under reduced pressure. The crude product was purified using method RPFC1 (10% to 100% gradient in 30 min), resulting in 4-bromo-1-(5-fluoro-2-methoxyphenyl)-2,5-diisopropyl-1H-imidazole (500 mg, 62%) as a dark oil. m/z: ES⁺ [M+H]⁺=356.95.

### Step 4: 1-(5-fluoro-2-methoxyphenyl)-2,5-diisopropyl-4-methyl-1H-imidazole

A mixture of 4-bromo-1-(5-fluoro-2-methoxyphenyl)-2,5-diisopropyl-1H-imidazole (500 mg, 1.4 mmol, 1.0 equiv), methylboronic acid (337 mg, 5.6 mmol, 4.0 equiv), Pd₂(dba)₃ (129 g, 0.14 mmol, 0.1 equiv), XPhos (101 mg, 0.21 mmol, 0.15 equiv) and tBuONa (406 mg, 4.2 mmol, 3.0 equiv) in dioxane (50 mL) was stirred at 100° C. for 5 h under nitrogen. The resulting mixture was then diluted with water (10 mL) and extracted with EtOAc (2×50 mL). The combined organic layers were washed with brine (10 mL), dried over anhydrous Na₂SO₄, and concentrated under reduced pressure. The crude product was purified using method RPFC1 (10% to 100% gradient in 30 min), resulting in 1-(5-fluoro-2-methoxyphenyl)-2,5-diisopropyl-4-methyl-1H-imidazole (400 mg, 98%) as a brown oil. m/z: ES⁺ [M+H]⁺=291.15.

### Step 5: 2-(2,5-diisopropyl-4-methyl-1H-imidazol-1-yl)-4-fluorophenol

A solution of 1-(5-fluoro-2-methoxyphenyl)-2,5-diisopropyl-4-methyl-1H-imidazole (350 mg, 1.2 mmol, 1.0 equiv) and BBr₃ (1 M in DCM, 12 mL, 12 mmol, 10 equiv) in DCM (1 mL) was stirred at 40° C. for 24 h under nitrogen. The resulting mixture was quenched with MeOH at 0° C., concentrated under reduced pressure and the crude product was purified using method RPFC1 (10% to 100% gradient in 30 min), to afford 2-(2,5-diisopropyl-4-methyl-1H-imidazol-1-yl)-4-fluorophenol (300 mg, 90%) as a brown solid. m/z: ES⁺ [M+H]⁺=277.25.

### 5-(5-fluoro-2-hydroxyphenyl)-4-isopropyl-1,6-dimethylpyridin-2(1H)-one

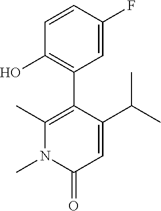 [SMILES: Cc1c(-c2cc(F)ccc2O)c(C(C)C)cc(=O)n1C]

### Step 1: 5-bromo-4-isopropyl-6-methylpyridin-2-amine

A mixture of 4-isopropyl-6-methylpyridin-2-amine (2.5 g, 17 mmol, 1.0 equiv) and NBS (3.0 g, 17 mmol, 1.0 equiv) in DMF (20 mL) was stirred at room temperature for 3 h under nitrogen. The resulting mixture was then diluted with water (50 mL) and extracted with EtOAc (2×50 mL). The combined organic layers were washed with water (2×50 mL), dried over anhydrous Na₂SO₄, and concentrated under reduced pressure. The residue was purified using method RPFC1 (10% to 100% gradient in 20 min), resulting in 5-bromo-4-isopropyl-6-methylpyridin-2-amine (2.5 g, 66%) as a brown solid. m/z: ES⁺ [M+H]⁺=230.05.

### Step 2: 3-bromo-6-fluoro-4-isopropyl-2-methylpyridine

Pyridine hydrofluoride (25 mL, 5.0 equiv) was added to a solution of 5-bromo-4-isopropyl-6-methylpyridin-2-amine (2.5 g, 11 mmol, 1.0 equiv) in pyridine (25 mL), and the mixture was stirred at 0° C. for 30 min. NaNO₂ (979 mg, 14 mmol, 1.3 equiv) was then added in portions at −20° C., and stirring continued at room temperature for 2 h under nitrogen. The resulting mixture was diluted with water (50 mL) and extracted with EtOAc (2×50 mL). The combined organic layers were washed with water (2×50 mL), dried over anhydrous Na₂SO₄, and concentrated under reduced pressure to afford 3-bromo-6-fluoro-4-isopropyl-2-methylpyridine (2.5 g, quant.) as a yellow oil. m/z: ES⁺ [M+H]⁺=233.15.

### Step 3: 6-fluoro-3-(5-fluoro-2-methoxyphenyl)-4-isopropyl-2-methylpyridine

A solution of 3-bromo-6-fluoro-4-isopropyl-2-methylpyridine (900 mg, 3.9 mmol, 1.0 equiv), (5-fluoro-2-methoxyphenyl)boronic acid (988 mg, 5.8 mmol, 1.5 equiv), RuPhos Pd G3 (648 mg, 0.8 mmol, 0.2 equiv), RuPhos (361 mg, 0.78 mmol, 0.2 equiv) and KOH (652 mg, 12 mmol, 3.0 equiv) in dioxane (20 mL) and H₂O (5 mL) was stirred at 100° C. for 16 h under nitrogen. The mixture was diluted with water (50 mL) and extracted with EtOAc (2×50 mL). The combined organic layers were washed with water (2×50 mL), dried over anhydrous Na₂SO₄ and concentrated under reduced pressure. The residue was purified using method RPFC1 (10% to 100% gradient in 20 min), to afford 6-fluoro-3-(5-fluoro-2-methoxyphenyl)-4-isopropyl-2-methylpyridine (700 mg, 65%) as a yellow solid. m/z: ES⁺ [M+H]⁺=278.25.

### Step 4: 5-(5-fluoro-2-methoxyphenyl)-4-isopropyl-6-methylpyridin-2(1H)-one

A mixture of 6-fluoro-3-(5-fluoro-2-methoxyphenyl)-4-isopropyl-2-methylpyridine (700 mg, 2.5 mmol, 1.0 equiv) and HOAc (5 mL) in H₂O (5 mL) was stirred at 100° C. for 16 h. The resulting mixture was concentrated under vacuum to afford 5-(5-fluoro-2-methoxyphenyl)-4-isopropyl-6-methylpyridin-2(1H)-one (700 mg, quant.) as a light-yellow oil. m/z: ES⁺ [M+H]⁺=276.30.

### Step 5: 5-(5-fluoro-2-methoxyphenyl)-4-isopropyl-1,6-dimethylpyridin-2(1H)-one

A mixture of 5-(5-fluoro-2-methoxyphenyl)-4-isopropyl-6-methylpyridin-2(1H)-one (500 mg, 1.8 mmol, 1.0 equiv), MeI (386 mg, 2.7 mmol, 1.5 equiv) and Cs₂CO₃ (1.8 g, 5.5 mmol, 3.0 equiv) in DMF (10 mL) was stirred at room temperature for 3 h. The resulting mixture was diluted with water (50 mL) and extracted with EtOAc (2×50 mL). The combined organic layers were washed with water (2×50 mL), dried over anhydrous Na₂SO₄, and concentrated under reduced pressure to afford 5-(5-fluoro-2-methoxyphenyl)-4-isopropyl-1,6-dimethylpyridin-2(1H)-one (450 mg, 86%) as a light-yellow oil. m/z: ES⁺ [M+H]⁺=290.15.

### Step 6: 5-(5-fluoro-2-hydroxyphenyl)-4-isopropyl-1,6-dimethylpyridin-2(1H)-one

A solution of 5-(5-fluoro-2-methoxyphenyl)-4-isopropyl-1,6-dimethylpyridin-2(1H)-one (430 mg, 1.5 mmol, 1.0 equiv) and BBr₃ (1 M in DCM, 5 mL, 5 mmol, 3.3 equiv) in DCM (0.5 mL) was stirred at room temperature for 3 h under nitrogen. The resulting mixture was quenched with MeOH at 0° C. and concentrated under reduced pressure. The residue was triturated with EtOAc (2×4 mL), and the resulting solid was collected by filtration and dried to afford 5-(5-fluoro-2-hydroxyphenyl)-4-isopropyl-1,6-dimethylpyridin-2(1H)-one (300 mg, 73%) as an off-white solid. m/z: ES⁺ [M+H]⁺=276.30.

### 1-(5-fluoro-2-hydroxyphenyl)-2,6-dimethylpyridin-4(1H)-one

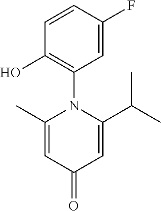 [SMILES: Cc1cc(=O)cc(C(C)C)n1-c1cc(F)ccc1O]

### Step 1: 1-(5-fluoro-2-methoxyphenyl)-2,6-dimethylpyridin-4(1H)-one

A solution of 5-fluoro-2-methoxy aniline (524 mg, 3.7 mmol, 0.9 equiv) and 2,6-dimethyl-4H-pyran-4-one (500 mg, 4.0 mmol, 1.0 equiv) in EtOH (2 mL)/water (6 mL) was irradiated in a microwave at 140° C. for 3 h. The resulting mixture was then cooled and concentrated under reduced pressure. The residue was purified using method RPFC1 (40% to 70% gradient in 10 min), to afford 1-(5-fluoro-2-methoxyphenyl)-2,6-dimethylpyridin-4(1H)-one (300 mg, 30%) as a brown solid. m/z: ES⁺ [M+H]⁺=248.10.

### Step 2: 1-(5-fluoro-2-hydroxyphenyl)-2,6-dimethylpyridin-4(1H)-one

A solution of 1-(5-fluoro-2-methoxyphenyl)-2,6-dimethylpyridin-4(1H)-one (290 mg, 1.2 mmol, 1.0 equiv) and BBr₃ (870 mg, 3.5 mmol, 3.0 equiv) in DCM (3 mL) was stirred at room temperature for 3 h under nitrogen. The resulting mixture was quenched with water (10 mL) and extracted with EtOAc (3×10 mL). The combined organic layers were washed with brine (5 mL), dried over anhydrous Na₂SO₄, and concentrated under reduced pressure. The residue was purified using method RPFC1 (10% to 50% gradient in 10 min), to afford 1-(5-fluoro-2-hydroxyphenyl)-2,6-dimethylpyridin-4(1H)-one (170 mg, 62%) as a yellow oil. m/z: ES⁺ [M+H]⁺=234.05.

### 1-(5-fluoro-2-hydroxyphenyl)-2,6-diisopropylpyridin-4(1H)-one

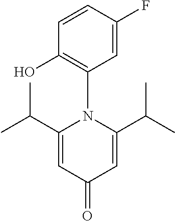 [SMILES: CC(C)c1cc(=O)cc(C(C)C)n1-c1cc(F)ccc1O]

### Step 1: 1-(5-fluoro-2-methoxyphenyl)-2,6-diisopropylpyridin-4(1H)-one

A solution of 5-fluoro-2-methoxyaniline (500 mg, 3.5 mmol, 1.0 equiv) and 2,8-dimethylnonane-3,5,7-trione (800 mg, 4.0 mmol, 1.1 equiv) in HOAc (3 mL)/EtOH (9 mL) was irradiated in a microwave at 140° C. for 3 h. The resulting mixture was then cooled and concentrated under reduced pressure. The residue was purified using method RPFC1 (30% to 80% gradient in 10 min), to afford 1-(5-fluoro-2-methoxyphenyl)-2,6-diisopropylpyridin-4(1H)-one (170 mg, 16%) as a yellow solid. m/z: ES⁺ [M+H]⁺=304.15.

### Step 2: 1-(5-fluoro-2-hydroxyphenyl)-2,6-diisopropylpyridin-4(1H)-one

A solution of 1-(5-fluoro-2-methoxyphenyl)-2,6-diisopropylpyridin-4(1H)-one (150 mg, 0.49 mmol, 1.0 equiv) and BBr₃ (900 mg, 3.6 mmol, 7.3 equiv) in DCM (0.5 mL) was stirred at 40° C. for 16 h under nitrogen. The resulting mixture was quenched with MeOH (3 mL) at 0° C., diluted with water (10 mL) and extracted with DCM (3×10 mL). The combined organic layers were washed with brine (5 mL), dried over anhydrous Na₂SO₄, and concentrated under reduced pressure. The residue was purified using method RPFC1 (10% to 50% gradient in 10 min), to afford 1-(5-fluoro-2-hydroxyphenyl)-2,6-diisopropylpyridin-4(1H)-one (70 mg, 49%) as a yellow oil. m/z: ES⁺ [M+H]⁺=290.20.

### 4-fluoro-2-(2-methyl-6,7-dihydro-5H-pyrrolo[1,2-a]imidazol-3-yl)phenol

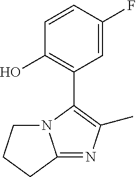 [SMILES: Cc1nc2n(c1-c1cc(F)ccc1O)CCC2]

### Step 1: 3-(2-(benzyloxy)-5-fluorophenyl)-6,7-dihydro-5H-pyrrolo[1,2-a]imidazole

A mixture of (2-(benzyloxy)-5-fluorophenyl)boronic acid (5.1 g, 216 mmol, 1.0 equiv), 3-bromo-6,7-dihydro-5H-pyrrolo[1,2-a]imidazole (3.9 g, 21 mmol, 1.0 equiv), Pd(dppf)Cl₂CH₂Cl₂ (3.4 g, 4.2 mmol, 0.2 equiv) and K₂CO₃ (5.8 g, 42 mmol, 2.0 equiv) in dioxane (40 mL)/water (4 mL) was stirred 100° C. for 3 h under nitrogen. The resulting mixture was diluted with water (100 mL) and extracted with DCM (3×100 mL). The combined organic layers were washed with brine (2×200 mL), dried over anhydrous Na₂SO₄, and concentrated under reduced pressure. The residue was purified by silica gel column chromatography, eluting with EtOAc to afford 3-(2-(benzyloxy)-5-fluorophenyl)-6,7-dihydro-5H-pyrrolo[1,2-a]imidazole (4.7 g, 73%) as a dark brown solid. m/z: ES⁺ [M+H]⁺=309.05.

### Step 2: 3-(2-(benzyloxy)-5-fluorophenyl)-2-bromo-6,7-dihydro-5H-pyrrolo[1,2-a]imidazole

A mixture of 3-(2-(benzyloxy)-5-fluorophenyl)-6,7-dihydro-5H-pyrrolo[1,2-a]imidazole (53.7 g, 12 mmol, 1.0 equiv) and NBS (2.1 g, 12 mmol, 1.0 equiv) in MeCN (40 mL) was stirred at room temperature for 3 h under nitrogen. The resulting mixture was then diluted with water (200 mL), and extracted with DCM (3×200 mL). The combined organic layers were washed with brine (2×200 mL), dried over anhydrous Na₂SO₄, and concentrated under reduced pressure. The residue was purified using method RPFC1 (10% to 100% gradient in 30 min), resulting in 3-(2-(benzyloxy)-5-fluorophenyl)-2-bromo-6,7-dihydro-5H-pyrrolo[1,2-a]imidazole (2.7 g, 58%) as a dark brown solid. m/z: ES⁺ [M+H]⁺=387.05.

### Step 3: 3-(2-(benzyloxy)-5-fluorophenyl)-2-methyl-6,7-dihydro-5H-pyrrolo[1,2-a]imidazole

A mixture of 3-(2-(benzyloxy)-5-fluorophenyl)-2-bromo-6,7-dihydro-5H-pyrrolo[1,2-a]imidazole (709 mg, 1.8 mmol, 1.0 equiv), methylboronic acid (329 mg, 5.5 mmol, 3.0 equiv), tBuONa (528 mg, 5.5 mmol, 3.0 equiv), XPhos (175 mg, 0.37 mmol, 0.2 equiv) and Pd₂(dba)₃ (335 mg, 0.37 mmol, 0.2 equiv) in dioxane (10 mL) was stirred at 90° C. for 2 h under nitrogen. The resulting mixture was diluted with water (30 mL) and extracted with DCM (3×30 mL). The combined organic layers were washed with brine (2×30 mL), dried over anhydrous Na₂SO₄, and concentrated under reduced pressure. The residue was purified using method RPFC1 (10% to 100% gradient in 25 min), resulting in 3-(2-(benzyloxy)-5-fluorophenyl)-2-methyl-6,7-dihydro-5H-pyrrolo[1,2-a]imidazole (500 mg, 85%) as a brown oil. m/z: ES⁺ [M+H]⁺=323.15.

### Step 4: 4-fluoro-2-(2-methyl-6,7-dihydro-5H-pyrrolo[1,2-a]imidazol-3-yl)phenol

A mixture of 13-(2-(benzyloxy)-5-fluorophenyl)-2-methyl-6,7-dihydro-5H-pyrrolo[1,2-a]imidazole (470 mg, 1.5 mmol, 1.0 equiv) and Pd/C (10% wt, 0.60 g, wet) in MeOH (5 mL) was stirred at room temperature for 16 h under hydrogen. The resulting mixture was filtered through Celite and concentrated under reduced pressure to afford 4-fluoro-2-(2-methyl-6,7-dihydro-5H-pyrrolo[1,2-a]imidazol-3-yl)phenol (310 mg, quant.) as alight brown solid. m/z: ES⁺ [M+H]⁺=233.10.

### 4-fluoro-2-(2-isopropyl-6,7-dihydro-5H-pyrrolo[1,2-a]imidazol-3-yl)phenol

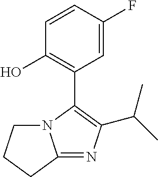 [SMILES: CC(C)c1nc2n(c1-c1cc(F)ccc1O)CCC2]

### Step 1: 3-(2-(benzyloxy)-5-fluorophenyl)-2-(prop-1-en-2-yl)-6,7-dihydro-5H-pyrrolo[1,2-a]imidazole

A mixture of 3-(2-(benzyloxy)-5-fluorophenyl)-2-bromo-6,7-dihydro-5H-pyrrolo[1,2-a]imidazole (1.0 g, 2.6 mmol, 1.0 equiv), 4,4,5,5-tetramethyl-2-(prop-1-en-2-yl)-1,3,2-dioxaborolane (1.3 g, 7.5 mmol, 3.0 equiv), Pd(dppf)Cl₂CH₂Cl₂ (0.21 g, 0.26 mmol, 0.1 equiv) and K₂CO₃ (0.71 g, 5.2 mmol, 2.0 equiv) in dioxane (10 mL) and water (20 mL) was stirred at 90° C. for 16 h under nitrogen. The resulting mixture was diluted with water (20 mL) and extracted with EtOAc (3×20 mL). The combined organic layers were dried over anhydrous Na₂SO₄ and concentrated under reduced pressure. The residue was purified using method RPFC1 (0% to 80% gradient in 20 min) to afford 3-(2-(benzyloxy)-5-fluorophenyl)-2-(prop-1-en-2-yl)-6,7-dihydro-5H-pyrrolo[1,2-a]imidazole (400 mg, 44%) as a yellow green solid. m/z: ES⁺ [M+H]⁺=349.16.

### Step 2: 4-fluoro-2-(2-isopropyl-6,7-dihydro-5H-pyrrolo[1,2-a]imidazol-3-yl)phenol

A mixture of 3-(2-(benzyloxy)-5-fluorophenyl)-2-(prop-1-en-2-yl)-6,7-dihydro-5H-pyrrolo[1,2-a]imidazole (380 mg, 1.1 mmol, 1.0 equiv) and Pd/C (10% wt, 100 mg, wet) in MeOH (4 mL) was stirred at room temperature for 2 h under hydrogen. The resulting mixture was filtered, and the filter cake was washed with MeOH (3×3 mL). The filtrate was concentrated under reduced pressure to afford 4-fluoro-2-(2-isopropyl-6,7-dihydro-5H-pyrrolo[1,2-a]imidazol-3-yl)phenol (200 mg, 70%) as a light-yellow solid. m/z: ES⁺ [M+H]⁺=261.13.

### 4-fluoro-2-(4-isopropyl-1,2-dimethyl-1H-imidazol-5-yl)phenol

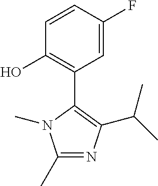 [SMILES: Cc1nc(C(C)C)c(-c2cc(F)ccc2O)n1C]

### Step 1: 5-(2-(benzyloxy)-5-fluorophenyl)-1,2-dimethyl-1H-imidazole

A mixture of (2-(benzyloxy)-5-fluorophenyl)boronic acid (6.3 g, 26 mmol, 3.0 equiv), 5-bromo-1,2-dimethyl-1H-imidazole (1.5 g, 8.6 mmol, 1.0 equiv), Pd(dppf)Cl₂CH₂Cl₂ (0.7 g, 0.86 mmol, 0.1 equiv) and K₂CO₃ (2.4 g, 17 mmol, 2.0 equiv) in dioxane (18 mL)/water (2 mL) was stirred at 100° C. for 16 h under nitrogen. The resulting mixture was diluted with water (20 mL), extracted with EtOAc (2×20 mL), dried over anhydrous Na₂SO₄, and concentrated under reduced pressure. The residue was purified using method RPFC2 (10% to 100% gradient in 30 min), resulting in 5-(2-(benzyloxy)-5-fluorophenyl)-1,2-dimethyl-1H-imidazole (1.5 g, 59%) as a light-yellow solid. m/z: ES⁺ [M+H]⁺=297.13.

### Step 2: 5-(2-(benzyloxy)-5-fluorophenyl)-4-bromo-1,2-dimethyl-1H-imidazole

A mixture of 5-(2-(benzyloxy)-5-fluorophenyl)-1,2-dimethyl-1H-imidazole (1.5 g, 5.3 mmol, 1.0 equiv) and NBS (601 mg, 5.1 mmol, 1.0 equiv) in MeCN (15 mL) was stirred at room temperature for 2 h under nitrogen. The resulting mixture was then diluted with water (20 mL) and extracted with DCM (3×15 mL). The combined organic layers were dried over anhydrous Na₂SO₄ and concentrated under reduced pressure to give 5-(2-(benzyloxy)-5-fluorophenyl)-4-bromo-1,2-dimethyl-1H-imidazole (1.5 g, 79%) as a dark red solid. m/z: ES⁺ [M+H]⁺=375.04.

### Step 3: 5-(2-(benzyloxy)-5-fluorophenyl)-1,2-dimethyl-4-(prop-1-en-2-yl)-1H-imidazole

A mixture of 5-(2-(benzyloxy)-5-fluorophenyl)-4-bromo-1,2-dimethyl-1H-imidazole (2.0 g, 5.3 mmol, 1.0 equiv), 4,4,5,5-tetramethyl-2-(prop-1-en-2-yl)-1,3,2-dioxaborolane (2.7 g, 16 mmol, 3.0 equiv), Pd(dppf)Cl₂CH₂Cl₂ (0.44 g, 0.53 mmol, 0.1 equiv) and K₂CO₃ (1.5 g, 11 mmol, 2.0 equiv) in dioxane (18 mL) and water (2 mL) was stirred at 100° C. for 16 h under nitrogen. The resulting mixture was diluted with water (20 mL) and extracted with EtOAc (3×20 mL). The combined organic layers were dried over anhydrous Na₂SO₄ and concentrated under reduced pressure. The residue was purified using method RPFC2 (0% to 80% gradient in 10 min) to afford 5-(2-(benzyloxy)-5-fluorophenyl)-1,2-dimethyl-4-(prop-1-en-2-yl)-1H-imidazole (1.1 g, 61%) as a light-yellow solid. m/z: ES⁺ [M+H]⁺=337.16.

### Step 4: 4-fluoro-2-(4-isopropyl-1,2-dimethyl-1H-imidazol-5-yl)phenol

A mixture of 5-(2-(benzyloxy)-5-fluorophenyl)-1,2-dimethyl-4-(prop-1-en-2-yl)-1H-imidazole (1.0 g, 3.0 mmol, 1.0 equiv) and Pd/C (10% wt, 158 mg, wet) in MeOH (10 mL) was stirred at room temperature for 3 h under hydrogen. The resulting mixture was filtered through Celite, the filter cake was washed with MeOH (3×5 mL).

The filtrate was concentrated under reduced pressure to afford 4-fluoro-2-(4-isopropyl-1,2-dimethyl-1H-imidazol-5-yl)phenol (700 mg, 95%) as alight-yellow solid. m/z: ES⁺ [M+H]⁺=249.13.

### 4-fluoro-2-(1-isopropyl-2,5-dimethyl-1H-imidazol-4-yl)phenol

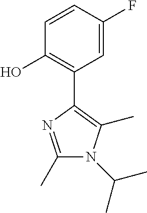 [SMILES: Cc1nc(-c2cc(F)ccc2O)c(C)n1C(C)C]

### Step 1: 4-(2-(benzyloxy)-5-fluorophenyl)-1-isopropyl-2-methyl-1H-imidazole

A mixture of (2-(benzyloxy)-5-fluorophenyl)boronic acid (2.1 g, 8.4 mmol, 1.0 equiv), 4-bromo-1-isopropyl-2-methyl-1H-imidazole (1.7 g, 8.4 mmol, 1.0 equiv), Pd(dppf)Cl₂CH₂Cl₂ (1.0 g, 1.3 mmol, 0.15 equiv) and K₂CO₃ (3.5 g, 25 mmol, 3.0 equiv) in dioxane (18 mL)/water (6 mL) was stirred at 100° C. for 2 h under nitrogen. The resulting mixture was extracted with EtOAc (3×50 mL), dried over anhydrous Na₂SO₄, and concentrated under reduced pressure. The residue was purified using method RPFC1 (10% to 100% gradient in 20 min), resulting in 4-(2-(benzyloxy)-5-fluorophenyl)-1-isopropyl-2-methyl-1H-imidazole (1.0 g, 37%) as a brown solid. m/z: ES⁺ [M+H]⁺=325.05.

### Step 2: 4-(2-(benzyloxy)-5-fluorophenyl)-5-bromo-1-isopropyl-2-methyl-1H-imidazole

A mixture of 4-(2-(benzyloxy)-5-fluorophenyl)-1-isopropyl-2-methyl-1H-imidazole (1.0 g, 3.1 mmol, 1.0 equiv) and NBS (550 mg, 3.1 mmol, 1.0 equiv) in DMF (10 mL) was stirred at room temperature for 2 h under nitrogen. The resulting mixture was then extracted with DCM (3×20 mL). The combined organic layers were dried over anhydrous Na₂SO₄, and concentrated under reduced pressure. The residue was purified using method RPFC1 (10% to 100% gradient in 20 min), to give 4-(2-(benzyloxy)-5-fluorophenyl)-5-bromo-1-isopropyl-2-methyl-1H-imidazole (560 mg, 45%) as a brown oil. m/z: ES⁺ [M+H]⁺=404.85.

### Step 3: 4-(2-(benzyloxy)-5-fluorophenyl)-1-isopropyl-2,5-dimethyl-1H-imidazole

A mixture of 4-(2-(benzyloxy)-5-fluorophenyl)-5-bromo-1-isopropyl-2-methyl-1H-imidazole (450 mg, 1.1 mmol, 1.0 equiv), methylboronic acid (267 mg, 4.5 mmol, 4.0 equiv), Pd₂(dba)₃ (102 mg, 0.11 mmol, 0.1 equiv), XPhos (80 mg, 0.17 mmol, 0.15 equiv) and tBuONa (322 mg, 3.3 mmol, 3.0 equiv) in dioxane (5 mL) was stirred at 100° C. for 1 h under nitrogen. The resulting mixture was then extracted with EtOAc (3×20 mL). The combined organic layers were washed with brine (2×10 mL), dried over anhydrous Na₂SO₄, and concentrated under reduced pressure. The residue was purified using method RPFC1 (10% to 100% gradient in 20 min), resulting in 4-(2-(benzyloxy)-5-fluorophenyl)-1-isopropyl-2,5-dimethyl-1H-imidazole (400 mg, 45%) as a brown oil. m/z: ES⁺ [M+H]⁺=339.15.

### Step 4: 4-fluoro-2-(1-isopropyl-2,5-dimethyl-1H-imidazol-4-yl)phenol

A mixture of 4-(2-(benzyloxy)-5-fluorophenyl)-1-isopropyl-2,5-dimethyl-1H-imidazole (400 mg, 1.2 mmol, 1.0 equiv) and Pd/C (10% wt, 200 mg, wet) in MeOH (2 mL) was stirred at room temperature for 2 h under hydrogen. The resulting mixture was filtered through Celite, the filter cake was washed with MeOH (3×10 mL). The filtrate was concentrated under reduced pressure to afford 4-fluoro-2-(1-isopropyl-2,5-dimethyl-1H-imidazol-4-yl)phenol (300 mg, quant.) as a brown oil. m/z: ES⁺ [M+H]⁺=249.10.

### 6-(5-fluoro-2-hydroxyphenyl)-5-isopropyl-1,4-dimethylpyridin-2(1H)-one

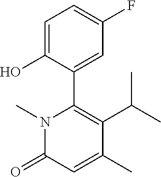 [SMILES: Cc1cc(=O)n(C)c(-c2cc(F)ccc2O)c1C(C)C]

### Step 1: methyl 4-(5-fluoro-2-methoxybenzoyl)-3,5-dimethylhexanoate

tBuONa (11 g, 105 mmol, 1.1 equiv) was added to a stirred solution of 1-(5-fluoro-2-methoxyphenyl)-3-methylbutan-1-one (cAS 1094648-17-5) (20 g, 95 mmol, 1.0 equiv) and methyl (E)-but-2-enoate (10.5 g, 105 mmol, 1.1 equiv) in THF (200 mL) at 0° C. The resulting mixture was stirred at 30° C. for 16 h and then was concentrated under reduced pressure. The residue was purified by silica gel column chromatography, eluting with PE/EtOAc (3:1) to afford methyl 4-(5-fluoro-2-methoxybenzoyl)-3,5-dimethylhexanoate (12.3 g, 42%) as a yellow oil. m/z: ES⁺ [M+H]⁺=311.05.

### Step 2: 4-(5-fluoro-2-methoxybenzoyl)-3,5-dimethylhexanoic acid

A solution of methyl 4-(5-fluoro-2-methoxybenzoyl)-3,5-dimethylhexanoate (3.9 g, 13 mmol, 1.0 equiv) and NaOH (2 N, 39 mmol, 3.0 equiv) in THF (50 mL) was stirred at 40° C. for 1.5 days. The mixture was acidified to pH 3-4 with HCl (aq.) at 0° C. and extracted with DCM (2×100 mL). The combined organic layers were washed with brine (200 mL), dried over anhydrous Na₂SO₄, and concentrated under reduced pressure. The residue was purified using method RPFC1 (10% to 65% gradient in 25 min), resulting in 4-(5-fluoro-2-methoxybenzoyl)-3,5-dimethylhexanoic acid (1.9 g, 51%) as a brown yellow solid. m/z: ES⁺ [M+H]⁺=297.10.

### Step 3: 4-(5-fluoro-2-methoxybenzoyl)-N,3,5-trimethylhexanamide

Methylamine (30% in EtOH, 5 mL) was added to a stirred solution of 4-(5-fluoro-2-methoxybenzoyl)-3,5-dimethylhexanoic acid (1.8 g, 6.0 mmol, 1.0 equiv), DIEA (2.3 g, 18 mmol, 3.0 equiv) and HATU (2.3 g, 6.0 mmol, 1.0 equiv) in DMF (20 mL) at room temperature. The resulting mixture was stirred at 65° C. for 16 h, and then cooled and purified using method RPFC1 (10% to 85% gradient in 25 min), resulting in 4-(5-fluoro-2-methoxybenzoyl)-N,3,5-trimethylhexanamide (1.3 g, 68%) as a light-yellow solid. m/z: ES⁺ [M+H]⁺=310.10.

### Step 4: 6-(5-fluoro-2-methoxyphenyl)-5-isopropyl-1,4-dimethyl-3,4-dihydropyridin-2(1H)-one

A solution of 4-(5-fluoro-2-methoxybenzoyl)-N,3,5-trimethylhexanamide (1.3 g, 4.1 mmol, 1.0 equiv) and TsOH (702 mg, 4.1 mmol, 1.0 equiv) in toluene (15 mL) was stirred at 100° C. for 16 h. The resulting mixture was diluted with water (50 mL) and extracted with EtOAc (2×50 mL). The combined organic layers were washed with water (2×50 mL), dried over anhydrous Na₂SO₄, and concentrated under reduced pressure. The residue was purified using method RPFC1 (10% to 100% gradient in 25 min), resulting in 6-(5-fluoro-2-methoxyphenyl)-5-isopropyl-1,4-dimethyl-3,4-dihydropyridin-2(1H)-one (755 mg, 63%) as a yellow oil. m/z: ES⁺ [M+H]⁺=292.10.

### Step 5: 6-(5-fluoro-2-methoxyphenyl)-5-isopropyl-1,4-dimethylpyridin-2(1H)-one

A solution of 6-(5-fluoro-2-methoxyphenyl)-5-isopropyl-1,4-dimethyl-3,4-dihydropyridin-2(1H)-one (725 mg, 2.5 mmol, 1.0 equiv) and 4,5-dichloro-3,6-dioxocyclohexa-1,4-diene-1,2-dicarbonitrile (1.4 g, 6.2 mmol, 2.5 equiv) in toluene (10 mL) was stirred at 100° C. for 16 h. The resulting mixture was diluted with water (50 mL) and extracted with EtOAc (2×50 mL). The combined organic layers were washed with water (2×50 mL), dried over anhydrous Na₂SO₄, and concentrated under reduced pressure. The residue was purified using method RPFC1 (10% to 100% gradient in 25 min), resulting in 6-(5-fluoro-2-methoxyphenyl)-5-isopropyl-1,4-dimethylpyridin-2(11)-one (585 mg, 81%) as a brown solid. m/z: ES⁺ [M+H]⁺=290.05.

### Step 6: 6-(5-fluoro-2-hydroxyphenyl)-5-isopropyl-1,4-dimethylpyridin-2(1H)-one

A solution of 6-(5-fluoro-2-methoxyphenyl)-5-isopropyl-1,4-dimethylpyridin-2(11)-one (540 mg, 1.9 mmol, 1.0 equiv) in BBr₃ (5 mL)/DCM (0.5 mL) was stirred at room temperature for 16 h under nitrogen. The resulting mixture was quenched with MeOH at 0° C. and concentrated under reduced pressure. The residue was purified using method RPFC1 (10% to 100% gradient in 25 min), to afford 6-(5-fluoro-2-hydroxyphenyl)-5-isopropyl-1,4-dimethylpyridin-2(1H)-one (500 mg, 97%) as a brown solid. m/z: ES⁺ [M+H]⁺=276.10.

### 5-(5-fluoro-2-hydroxyphenyl)-6-isopropyl-1-methylpyridin-2(1H)-one

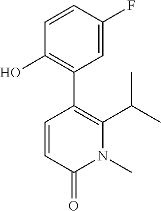 [SMILES: CC(C)c1c(-c2cc(F)ccc2O)ccc(=O)n1C]

### Step 1: 5-(2-(benzyloxy)-5-fluorophenyl)-6-chloro-1-methylpyridin-2(1H)-one

A mixture of 2-(benzyloxy)-5-fluorophenylboronic acid (1.7 g, 6.7 mmol, 1.0 equiv), 5-bromo-6-chloro-1-methylpyridin-2-one (1.5 g, 6.7 mmol, 1.0 equiv), Pd(OAc)₂ (151 mg, 0.67 mmol, 0.1 equiv), tricyclohexylphosphane (189 mg, 0.67 mmol, 0.1 equiv) and K₃PO₄ (2.9 g, 13 mmol, 2.0 equiv) in toluene (50 mL) and H₂O (5 mL) was stirred at 100° C. for 2 h under nitrogen. The mixture was diluted with water and extracted with DCM (2×50 mL). The combined organic layers were washed with brine (2×50 mL), dried over anhydrous Na₂SO₄ and concentrated under reduced pressure. The residue was purified using method RPFC1 (30% to 80% gradient in 15 min), to afford 5-(2-(benzyloxy)-5-fluorophenyl)-6-chloro-1-methylpyridin-2(1H)-one (1.0 mg, 43%) as a yellow oil. m/z: ES⁺ [M+H]⁺=344.08.

### Step 2: 5-(2-(benzyloxy)-5-fluorophenyl)-1-methyl-6-(prop-1-en-2-yl)pyridin-2(1H)-one

A solution of 5-(2-(benzyloxy)-5-fluorophenyl)-6-chloro-1-methylpyridin-2(1H)-one (1.0 g, 2.9 mmol, 1.0 equiv), 4,4,5,5-tetramethyl-2-(prop-1-en-2-yl)-1,3,2-dioxaborolane (488 mg, 2.9 mmol, 1.0 equiv), RuPhos Pd G3 (243 mg, 0.29 mmol, 0.1 equiv), RuPhos (272 mg, 0.58 mmol, 0.2 equiv) and K₂CO₃ (804 mg, 5.8 mmol, 2.0 equiv) in toluene (25 mL) and H₂O (2.5 mL) was stirred at 100° C. for 16 h under nitrogen. The mixture was diluted with water and extracted with DCM (2×50 mL). The combined organic layers were washed with brine (2×50 mL), dried over anhydrous Na₂SO₄ and concentrated under reduced pressure. The residue was purified using method RPFC1 (30% to 80% gradient in 15 min), to afford 5-(2-(benzyloxy)-5-fluorophenyl)-1-methyl-6-(prop-1-en-2-yl)pyridin-2(1H)-one (500 mg, 49%) as a yellow solid. m/z: ES⁺ [M+H]⁺=350.15.

### Step 3: 5-(5-fluoro-2-hydroxyphenyl)-6-isopropyl-1-methylpyridin-2(1H)-one

A solution of 5-(2-(benzyloxy)-5-fluorophenyl)-1-methyl-6-(prop-1-en-2-yl)pyridin-2(1H)-one (500 mg, 1.4 mmol, 1.0 equiv), Pd/C (150 mg, 10% wt, wet) and AcOH (86 mg, 1.4 mmol, 1.0 equiv) in EtOAc (5 mL) was stirred at room temperature for 16 h under hydrogen. The resulting mixture was then filtered and the filter cake was washed with EtOAc (3×30 mL). The filtrate was then concentrated under reduced pressure and purified using method RPFC1 (30% to 80% gradient in 20 min) to afford 5-(5-fluoro-2-hydroxyphenyl)-6-isopropyl-1-methylpyridin-2(1H)-one (300 mg, 80%) as a yellow solid. m/z: ES⁺ [M+H]⁺=262.12.

### 4-fluoro-2-(5-isopropyl-6-methylpyridazin-4-yl)phenol

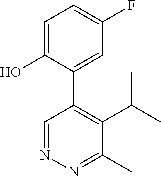 [SMILES: Cc1nncc(-c2cc(F)ccc2O)c1C(C)C]

### Step 1: 2-benzyl-4-chloro-5-(5-fluoro-2-methoxyphenyl)pyridazin-3(2H)-one

A mixture of 2-benzyl-5-bromo-4-chloropyridazin-3(2H)-one (4.5 g, 15 mmol, 1.0 equiv), (5-fluoro-2-methoxyphenyl)boronic acid (2.6 g, 15 mmol, 1.0 equiv), Pd(dppf)Cl₂CH₂Cl₂ (2.5 g, 3.0 mmol, 0.2 equiv), and K₂CO₃ (4.1 g, 30 mmol, 2.0 equiv) in dioxane (10 mL) and H₂O (2 mL) was stirred at 40° C. for 16 h under nitrogen. The mixture was diluted with water (100 mL) and extracted with DCM (3×100 mL). The combined organic layers were washed with brine (2×100 mL), dried over anhydrous Na₂SO₄ and concentrated under reduced pressure. The residue was purified using silica gel column chromatography, eluting with PE/EtOAc (16:1) to afford 2-benzyl-4-chloro-5-(5-fluoro-2-methoxyphenyl)pyridazin-3(2H)-one (4.0 g, 77%) as an off white solid. m/z: ES⁺ [M+H]⁺=345.00.

### Step 2: 2-benzyl-5-(5-fluoro-2-methoxyphenyl)-4-(prop-1-en-2-yl)pyridazin-3(2H)-one

A mixture of 2-benzyl-4-chloro-5-(5-fluoro-2-methoxyphenyl)pyridazin-3(2H)-one (4.0 g, 12 mmol, 1.0 equiv), 4,4,5,5-tetramethyl-2-(prop-1-en-2-yl)-1,3,2-dioxaborolane (2.0 g, 12 mmol, 1.0 equiv), Pd(dppf)Cl₂ (1.9 g, 2.3 mmol, 0.2 equiv) and K₂CO₃ (3.2 g, 23 mmol, 2.0 equiv) in dioxane (45 mL) and water (4.5 mL) was stirred at 100° C. for 16 h under nitrogen. The mixture was diluted water (100 mL) and extracted with DCM (3×100 mL). The combined organic layers were washed with brine (2×100 mL), dried over anhydrous Na₂SO₄ and concentrated under reduced pressure. The residue was purified using silica gel column chromatography, eluting with PE/EtOAc (1:1) to afford 2-benzyl-5-(5-fluoro-2-methoxyphenyl)-4-(prop-1-en-2-yl)pyridazin-3(2H)-one (3.0 g, 94%) as an orange oil. m/z: ES⁺ [M+H]⁺=351.05.

### Step 3: 2-benzyl-5-(5-fluoro-2-methoxyphenyl)-4-isopropylpyridazin-3(2H)-one

A solution of 2-benzyl-5-(5-fluoro-2-methoxyphenyl)-4-(prop-1-en-2-yl)pyridazin-3(2H)-one (750 mg, 2.1 mmol, 1.0 equiv), Pd/C (500 mg, 10% wt, wet) and AcOH (100 mg, 1.7 mmol, 0.8 equiv) in MeOH (10 mL) was stirred at room temperature for 16 h under 10 PSI hydrogen. The resulting mixture was then filtered through Celite and the filtrate was concentrated under reduced pressure to afford crude 2-benzyl-5-(5-fluoro-2-methoxyphenyl)-4-isopropylpyridazin-3(2H)-one, which was used without further purification. m/z: ES⁺ [M+H]⁺=353.20.

### Step 4: 5-(5-fluoro-2-methoxyphenyl)-4-isopropylpyridazin-3(2H)-one

A solution of 2-benzyl-5-(5-fluoro-2-methoxyphenyl)-4-(prop-1-en-2-yl)pyridazin-3(2H)-one (501 mg, 1.4 mmol, 1.0 equiv) and AlCl₃ (474 mg, 3.6 mmol, 2.5 equiv) in toluene (5 mL) was stirred at 80° C. for 30 min. The mixture was allowed to cool down to room temperature and then concentrated under reduced pressure. The residue was purified using method RPFC1 (10% to 100% gradient in 25 min) to afford 5-(5-fluoro-2-methoxyphenyl)-4-isopropylpyridazin-3(2H)-one (220 mg, 62%) as an off-white solid. m/z: ES⁺ [M+H]⁺=263.05.

### Step 5: 3-chloro-5-(5-fluoro-2-methoxyphenyl)-4-isopropylpyridazine

A solution of 5-(5-fluoro-2-methoxyphenyl)-4-isopropylpyridazin-3(2H)-one (200 mg, 0.76 mmol, 1.0 equiv) in POCl₃ (5 mL) was stirred at 100° C. for 3 h. The resulting mixture was concentrated under reduced pressure and the crude product was used without further purification. m/z: ES⁺ [M+H]⁺=281.05.

### Step 6: 5-(5-fluoro-2-methoxyphenyl)-4-isopropyl-3-methylpyridazine

A mixture of 3-chloro-5-(5-fluoro-2-methoxyphenyl)-4-isopropylpyridazine (215 mg, 0.77 mmol, 1.0 equiv), methylboronic acid (138 mg, 2.3 mmol, 3.0 equiv), Pd₂(dba)₃ (140 mg, 0.15 mmol, 0.2 equiv), XPhos (73 mg, 0.15 mmol, 0.2 equiv) and tBuONa (147 mg, 1.5 mmol, 2.0 equiv) in dioxane (3 mL) was stirred at 100° C. for 16 h under nitrogen. The mixture was diluted with water (30 mL) and extracted with DCM (3×30 mL). The combined organic layers were washed with brine (2×50 mL), dried over anhydrous Na₂SO₄ and concentrated under reduced pressure. The residue was purified using method RPFC1 (10% to 100% gradient in 25 min), resulting in 5-(5-fluoro-2-methoxyphenyl)-4-isopropyl-3-methylpyridazine (100 mg, 50%) as a light brown oil. m/z: ES⁺ [M+H]⁺=261.10.

### Step 7: 4-fluoro-2-(5-isopropyl-6-methylpyridazin-4-yl)phenol

A solution of 5-(5-fluoro-2-methoxyphenyl)-4-isopropyl-3-methylpyridazine (90 mg, 0.35 mmol, 1.0 equiv) in BBr₃ (1 mL)/DCM (1.5 mL) was stirred at room temperature for 16 h under nitrogen. The resulting mixture was quenched with MeOH at 0° C. and concentrated under reduced pressure. The residue was purified using method RPFC1 (10% to 100% gradient in 25 min), to afford 4-fluoro-2-(5-isopropyl-6-methylpyridazin-4-yl)phenol (70 mg, 82%) as a light-yellow oil. m/z: ES⁺ [M+H]⁺=247.02.

### 4-fluoro-2-(5-isopropyl-3-methylpyridazin-4-yl)phenol

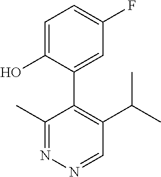 [SMILES: Cc1nncc(C(C)C)c1-c1cc(F)ccc1O]

### Step 1: 2-benzyl-4-(5-fluoro-2-methoxyphenyl)-5-isopropylpyridazin-3(2H)-one

A solution of 2-benzyl-4-(5-fluoro-2-methoxyphenyl)-5-(prop-1-en-2-yl)pyridazin-3(2H)-one (1.2 g, 3.4 mmol, 1.0 equiv), Pd/C (730 mg, 10% wt, wet) and AcOH (1 mL) in MeOH (20 mL) was stirred at room temperature for 16 h under hydrogen. The resulting mixture was then filtered through Celite, the filter cake was washed with DCM (3×10 mL), and the filtrate was concentrated under reduced pressure to afford 2-benzyl-4-(5-fluoro-2-methoxyphenyl)-5-isopropylpyridazin-3(2H)-one (900 mg, 75%) as a yellow oil. m/z: ES⁺ [M+H]⁺=353.15.

### Step 2: 4-(5-fluoro-2-methoxyphenyl)-5-isopropylpyridazin-3(2H)-one

A solution of 2-benzyl-4-(5-fluoro-2-methoxyphenyl)-5-isopropylpyridazin-3(2H)-one (900 mg, 2.6 mmol, 1.0 equiv) and AlCl₃ (851 mg, 6.4 mmol, 2.5 equiv) in toluene (10 mL) was stirred at 80° C. for 30 min. The mixture was allowed to cool down to room temperature and then concentrated under reduced pressure. The residue was purified using method RPFC1 (10% to 100% gradient in 10 min) to afford 4-(5-fluoro-2-methoxyphenyl)-5-isopropylpyridazin-3(21)-one (250 mg, 39%) as a colourless solid. m/z: ES⁺ [M+H]⁺=263.28.

### Step 3: 3-chloro-4-(5-fluoro-2-methoxyphenyl)-5-isopropylpyridazine

A solution of 4-(5-fluoro-2-methoxyphenyl)-5-isopropylpyridazin-3(2H)-one (250 mg, 0.95 mmol, 1.0 equiv) in POCl₃ (3 mL) was stirred at 100° C. for 2 h. The resulting mixture was concentrated under reduced pressure. The residue was purified using method RPFC1 (10% to 50% gradient in 10 min) to afford 3-chloro-4-(5-fluoro-2-methoxyphenyl)-5-isopropylpyridazine (220 mg, 82%) as a colourless solid. m/z: ES⁺ [M+H]⁺=281.25.

### Step 4: 4-(5-fluoro-2-methoxyphenyl)-5-isopropyl-3-methylpyridazine

A mixture of 3-chloro-4-(5-fluoro-2-methoxyphenyl)-5-isopropylpyridazine (220 mg, 0.78 mmol, 1.0 equiv), methylboronic acid (469 mg, 7.8 mmol, 10 equiv), Pd₂(dba)₃ (144 mg, 0.16 mmol, 0.2 equiv), XPhos (75 mg, 0.16 mmol, 0.2 equiv) and tBuONa (226 mg, 2.4 mmol, 3.0 equiv) in dioxane (3 mL) was stirred at 100° C. for 16 h under nitrogen. The mixture was diluted with water (10 mL) and extracted with DCM (3×5 mL). The combined organic layers were dried over anhydrous Na₂SO₄ and concentrated under reduced pressure. The residue was purified using method RPFC1 (10% to 50% gradient in 10 min), resulting in 4-(5-fluoro-2-methoxyphenyl)-5-isopropyl-3-methylpyridazine (190 mg, 93%) as a colourless solid. m/z: ES⁺ [M+H]⁺=261.10.

### Step 5: 4-fluoro-2-(5-isopropyl-3-methylpyridazin-4-yl)phenol

A solution of 4-(5-fluoro-2-methoxyphenyl)-5-isopropyl-3-methylpyridazine (200 mg, 0.77 mmol, 1.0 equiv) in BBr₃ (3 mL)/DCM (1 mL) was stirred at room temperature for 16 h under nitrogen. The resulting mixture was quenched with MeOH (20 mL) at 0° C. and concentrated under reduced pressure to give 4-fluoro-2-(5-isopropyl-3-methylpyridazin-4-yl)phenol (120 mg, 63%) as a light-yellow oil. m/z: ES⁺ [M+H]⁺=247.15.

### 4-fluoro-2-(1-isopropyl-2-methyl-1H-imidazol-5-yl)phenol

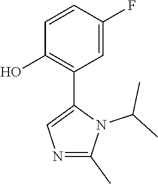 [SMILES: Cc1ncc(-c2cc(F)ccc2O)n1C(C)C]

### Step 1: 5-(2-(benzyloxy)-5-fluorophenyl)-1-isopropyl-2-methyl-1H-imidazole

A mixture of 5-bromo-1-isopropyl-2-methyl-1H-imidazole (1.8 g, 8.9 mmol, 1.0 equiv), (2-(benzyloxy)-5-fluorophenyl)boronic acid (2.2 g, 8.9 mmol, 1.0 equiv), Pd(dppf)Cl₂CH₂Cl₂ (1.5 g, 1.8 mmol, 0.2 equiv), and K₂CO₃ (3.7 g, 27 mmol, 3.0 equiv) in dioxane (18 mL)/H₂O (6 mL) was stirred at 100° C. for 2 h under nitrogen. The resulting mixture was extracted with EtOAc (3×30 mL). The combined organic layers were washed with brine (3×10 mL), dried over anhydrous Na₂SO₄, and concentrated under reduced pressure. The crude product was purified using method RPFC1 (10% to 100% gradient in 10 min), resulting in 5-(2-(benzyloxy)-5-fluorophenyl)-1-isopropyl-2-methyl-1H-imidazole (2.0 g, 70%) as a brown oil. m/z: ES⁺ [M+H]⁺=325.00.

### Step 2: 4-fluoro-2-(1-isopropyl-2-methyl-1H-imidazol-5-yl)phenol

A suspension of 5-(2-(benzyloxy)-5-fluorophenyl)-1-isopropyl-2-methyl-1H-imidazole (150 mg, 0.46 mmol, 1.0 equiv) and Pd/C (10% wt, wet, 70 mg) in MeOH (3 mL) was stirred at room temperature for 1 h under hydrogen. The resulting mixture was filtered, the filter cake was washed with MeOH (3×6 mL) and the filtrate was concentrated under reduced pressure to give 4-fluoro-2-(1-isopropyl-2-methyl-1H-imidazol-5-yl)phenol (100 mg, 92%) as a colourless solid. m/z: ES⁺ [M+H]⁺=235.15.

### 4-fluoro-2-(1-isopropyl-2,4-dimethyl-1H-imidazol-5-yl)phenol

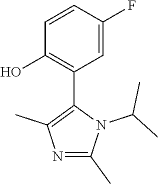 [SMILES: Cc1nc(C)n(C(C)C)c1-c1cc(F)ccc1O]

### Step 1: 5-(2-(benzyloxy)-5-fluorophenyl)-4-bromo-1-isopropyl-2-methyl-1H-imidazole

A mixture of 5-(2-(benzyloxy)-5-fluorophenyl)-1-isopropyl-2-methyl-1H-imidazole (1.0 g, 3.1 mmol, 1.0 equiv) and NBS (550 mg, 3.1 mmol, 1.0 equiv) in MeCN (10 mL) was stirred at room temperature for 3 h under nitrogen. The resulting mixture was then extracted with EtOAc (3×20 mL). The combined organic layers were washed with water (3×10 mL), dried over anhydrous Na₂SO₄, and concentrated under reduced pressure to give 5-(2-(benzyloxy)-5-fluorophenyl)-4-bromo-1-isopropyl-2-methyl-1H-imidazole (1.0 g, 80%) as a brown oil. m/z: ES⁺ [M+H]⁺=403.05.

### Step 2: 5-(2-(benzyloxy)-5-fluorophenyl)-1-isopropyl-2,4-dimethyl-1H-imidazole

A mixture of 5-(2-(benzyloxy)-5-fluorophenyl)-4-bromo-1-isopropyl-2-methyl-1H-imidazole (800 mg, 2.0 mmol, 1.0 equiv), methylboronic acid (238 mg, 4.0 mmol, 2.0 equiv), Pd₂(dba)₃ (363 mg, 0.40 mmol, 0.2 equiv), XPhos (189 mg, 0.40 mmol, 0.2 equiv) and tBuONa (572 mg, 6.0 mmol, 3.0 equiv) in dioxane (18 mL) was stirred at 100° C. for 1 h under nitrogen. The mixture was diluted with water (10 mL) and extracted with DCM (3×5 mL). The combined organic layers were dried over anhydrous Na₂SO₄ and concentrated under reduced pressure. The residue was purified using method RPFC1 (10% to 50% gradient in 10 min), resulting in 5-(2-(benzyloxy)-5-fluorophenyl)-1-isopropyl-2,4-dimethyl-TH-imidazole (150 mg, 22%) as abrown oil. m/z: ES⁺ [M+H]⁺=339.15.

### Step 3: 4-fluoro-2-(1-isopropyl-2,4-dimethyl-1H-imidazol-5-yl)phenol

A suspension of 5-(2-(benzyloxy)-5-fluorophenyl)-1-isopropyl-2,4-dimethyl-1H-imidazole (150 mg, 0.44 mmol, 1.0 equiv) and Pd/C (10% wt, wet, 75 mg) in MeOH (3 mL) was stirred at room temperature for 1 h under hydrogen. The resulting mixture was filtered and the filter cake was washed with MeOH (3×10 mL). The filtrate was concentrated under reduced pressure to give 4-fluoro-2-(1-isopropyl-2,4-dimethyl-1H-imidazol-5-yl)phenol (100 mg, 91%) as a colourless oil. m/z: ES⁺ [M+H]⁺=249.20.

### 2-(2-cyclopropyl-5-(trifluoromethyl)-1H-imidazol-1-yl)-4-fluorophenol

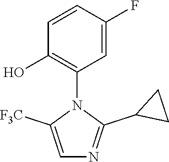 [SMILES: Oc1ccc(F)cc1-n1c(C(F)(F)F)cnc1C1CC1]

### Step 1: 2-cyclopropyl-1-(5-fluoro-2-methoxyphenyl)-5-(trifluoromethyl)-1H-imidazole

A solution of AgF (1.0 g, 7.75 mmol, 9.0 equiv) and TMSOTf (1.4 g, 9.5 mmol, 11 equiv) in DMF (50 mL) was stirred at room temperature for 2 h under nitrogen atmosphere. To the above mixture was added Cu (0.77 g, 12 mmol, 14 equiv) in portions at room temperature. The resulting mixture was stirred at room temperature for 6 h, after which 2-cyclopropyl-1-(5-fluoro-2-methoxy phenyl)imidazole (200 mg, 0.86 mmol, 1.0 equiv) was added in portions at room temperature. The resulting mixture was stirred at room temperature for an additional 2 h and at 90° C. for 16 h under nitrogen. The resulting mixture was diluted with water (100 mL) and extracted with EtOAc (3×50 mL). The combined organic layers were washed with brⁱne, dried over anhydrous Na₂SO₄, and concentrated under reduced pressure. The residue was purified using method RPFC1 (30% to 80% gradient in 10 min), resulting in 2-cyclopropyl-1-(5-fluoro-2-methoxyphenyl)-5-(trifluoromethyl)-1H-imidazole (100 mg, 32%) as a brown oil. m/z: ES⁺ [M+H]⁺=301.09.

### Step 2: 2-(2-cyclopropyl-5-(trifluoromethyl)-1H-imidazol-1-yl)-4-fluorophenol

A solution of 2-cyclopropyl-1-(5-fluoro-2-methoxyphenyl)-5-(trifluoromethyl)-1H-imidazole (100 mg, 0.33 mmol, 1.0 equiv) and BBr₃ (167 mg, 0.67 mmol, 2.0 equiv) in DCM (1 mL) was stirred at room temperature for 16 h. The reaction was quenched by the addition of MeOH at 0° C. and concentrated under reduced pressure to afford 2-(2-cyclopropyl-5-(trifluoromethyl)-1H-imidazol-1-yl)-4-fluorophenol (80 mg, 84%) as a light-yellow solid. m/z: ES⁺ [M+H]⁺=287.07.

### (S)-4-fluoro-2-((tetrahydrofuran-3-yl)oxy)phenol

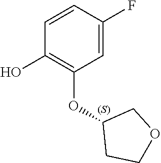

### Step 1: (S)-3-(2-(benzyloxy)-5-fluorophenoxy)tetrahydrofuran

A solution of 2-(benzyloxy)-5-fluorophenol (200 mg, 0.92 mmol, 1.0 equiv), PPh₃ (481 mg, 1.8 mmol, 2.0 equiv), (R)-tetrahydrofuran-3-ol (81 mg, 0.92 mmol, 1.0 equiv) and DIAD (371 mg, 1.8 mmol, 2.0 equiv) in THF (4 mL) was stirred at room temperature for 16 h under nitrogen. The resulting mixture was diluted with water (10 mL) and extracted with DCM (3×10 mL). The combined organic layers were washed with brine (2×10 mL), dried over anhydrous Na₂SO₄ and concentrated under reduced pressure. The residue was purified using method RPFC1 (30% to 80% gradient in 10 min), resulting in (S)-3-(2-(benzyloxy)-5-fluorophenoxy)tetrahydrofuran (190 mg, 72%) as a colourless solid. m/z: ES⁺ [M+H]⁺=289.12.

### Step 2: (S)-4-fluoro-2-((tetrahydrofuran-3-yl)oxy)phenol

A suspension of (S)-3-(2-(benzyloxy)-5-fluorophenoxy)tetrahydrofuran (190 mg, 0.66 mmol, 1.0 equiv) and Pd/C (70 mg, 0.66 mmol, 1.0 equiv) in MeOH (4 mL) was stirred at room temperature for 1 h under hydrogen. The resulting mixture was filtered, diluted with water (10 mL) and extracted with DCM (3×50 mL). The combined organic layers were washed with brine (30 mL), dried over anhydrous Na₂SO₄ and concentrated under reduced pressure. The residue was purified using method Prep-HPLC1 (24% to 36% gradient in 7 min, Rt=7.55 min), resulting in (S)-4-fluoro-2-((tetrahydrofuran-3-yl)oxy)phenol (140 mg, quant.) as a colourless solid. m/z: ES⁺ [M+H]⁺=199.07.

### (R)-4-fluoro-2-((tetrahydrofuran-3-yl)oxy)phenol

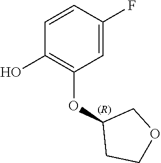

### Step 1: (R)-3-(2-(benzyloxy)-5-fluorophenoxy)tetrahydrofuran

A solution of 2-(benzyloxy)-5-fluorophenol (1.0 g, 4.6 mmol, 1.0 equiv), PPh₃ (1.9 g, 7.3 mmol, 1.6 equiv), (S)-tetrahydrofuran-3-ol (403 mg, 4.6 mmol, 1.0 equiv) and DIAD (1.5 g, 7.3 mmol, 1.6 equiv) in THF (20 mL) was stirred at room temperature for 4 h under nitrogen. The resulting mixture was diluted with water and extracted with EtOAc (3×30 mL). The combined organic layers were washed with brine (3×20 mL), dried over anhydrous Na₂SO₄ and concentrated under reduced pressure. The residue was purified using method RPFC1 (0% to 100% gradient in 30 min), resulting in (R)-3-(2-(benzyloxy)-5-fluorophenoxy)tetrahydrofuran (1.3 g, 98%) as a yellow oil. m/z: ES⁺ [M+H]⁺=289.05.

### Step 2: (R)-4-fluoro-2-((tetrahydrofuran-3-yl)oxy)phenol

A suspension of (R)-3-(2-(benzyloxy)-5-fluorophenoxy)tetrahydrofuran (170 mg, 0.59 mmol, 1.0 equiv) and Pd/C (10% wt, wet, 82 mg) in MeOH (3 mL) was stirred at room temperature for 4 h under hydrogen. The resulting mixture was filtered and the filter cake was washed with MeOH (3×5 mL). The filtrate was concentrated under reduced pressure, resulting in (R)-4-fluoro-2-((tetrahydrofuran-3-yl)oxy)phenol (100 mg, 86%) as a colourless solid. m/z: ES⁺ [M+H]⁺=199.15.

### 5-(5-fluoro-2-hydroxyphenyl)-6-isopropyl-4-methylpyridin-2(1H)-one

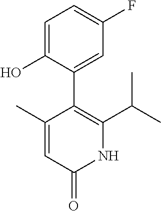 [SMILES: Cc1cc(=O)[nH]c(C(C)C)c1-c1cc(F)ccc1O]

### Step 1: 5-bromo-6-isopropyl-4-methylpyridin-2-amine

A mixture of 6-isopropyl-4-methylpyridin-2-amine (cAS 179555-17-0) (2.0 g, 13 mmol, 1.0 equiv) and NBS (2.4 g, 13 mmol, 1.0 equiv) in DMF (30 mL) was stirred at room temperature for 6 h under nitrogen. The resulting mixture was then diluted with water (100 mL) and extracted with EtOAc (3×100 mL). The combined organic layers were washed with water (3×100 mL), dried over anhydrous Na₂SO₄, and concentrated under reduced pressure. The residue was purified using method RPFC1 (40% to 90% gradient in 25 min) to give 5-bromo-6-isopropyl-4-methylpyridin-2-amine (1.6 g, 84%) as a light-yellow oil. m/z: ES⁺ [M+H]⁺=229.03.

### Step 2: 3-bromo-6-fluoro-2-isopropyl-4-methylpyridine

A solution of 5-bromo-6-isopropyl-4-methylpyridin-2-amine (1.6 g, 7.0 mmol, 1.0 equiv), NaNO₂ (964 mg, 14 mmol, 2.0 equiv) in pyridine hydrofluoride (5 mL, 0.017 mmol)/pyridine (5 mL) was stirred at room temperature for 16 h under nitrogen. The resulting mixture was diluted with water (20 mL) and extracted with EtOAc (3×10 mL). The combined organic layers were washed with water, dried over anhydrous Na₂SO₄, and concentrated under reduced pressure. This resulted in 3-bromo-6-fluoro-2-isopropyl-4-methylpyridine (900 mg, 56%) as a light-yellow solid. m/z: ES⁺ [M+H]⁺=232.01.

### Step 3: 6-fluoro-3-(5-fluoro-2-methoxyphenyl)-2-isopropyl-4-methylpyridine

A solution of 3-bromo-6-fluoro-2-isopropyl-4-methylpyridine (800 mg, 3.5 mmol, 1.0 equiv), 5-fluoro-2-methoxyphenylboronic acid (879 mg, 5.2 mmol, 1.5 equiv), CPhos Pd G3 (556 mg, 0.69 mmol, 0.2 equiv), CPhos (301 mg, 0.69 mmol, 0.2 equiv) and KOH (77 mg, 1.4 mmol, 0.4 equiv) in dioxane (10 mL)/H₂O (3 mL) was stirred at room temperature for 16 h under nitrogen. The resulting mixture was diluted with water (20 mL) and extracted with EtOAc (3×25 mL). The combined organic layers were washed with water, dried over anhydrous Na₂SO₄, and concentrated under reduced pressure. This resulted in 6-fluoro-3-(5-fluoro-2-methoxyphenyl)-2-isopropyl-4-methylpyridine (650 mg, 68%) as a light-yellow solid. m/z: ES⁺ [M+H]⁺=278.13.

### Step 4: 5-(5-fluoro-2-methoxyphenyl)-6-isopropyl-4-methylpyridin-2(1H)-one

A solution of 6-fluoro-3-(5-fluoro-2-methoxyphenyl)-2-isopropyl-4-methylpyridine (640 mg, 2.3 mmol, 1.0 equiv) in water (3 mL)/AcOH (10 mL) was stirred at 120° C. for 16 h. The resulting mixture was concentrated under reduced pressure, to afford 5-(5-fluoro-2-methoxyphenyl)-6-isopropyl-4-methylpyridin-2(1H)-one (500 mg, 79%) as a light-yellow oil. m/z: ES⁺ [M+H]⁺=276.13.

### Step 5: 3-(5-fluoro-2-methoxyphenyl)-2-isopropyl-6-methoxy-4-methylpyridine

A solution of 5-(5-fluoro-2-methoxyphenyl)-6-isopropyl-4-methylpyridin-2(1H)-one (200 mg, 0.73 mmol, 1.0 equiv), Cs₂CO₃ (474 mg, 1.5 mmol, 2.0 equiv), and MeI (155 mg, 1.1 mmol, 1.5 equiv) in DMF (8 mL) was stirred at room temperature for 2 h under nitrogen. The resulting mixture was diluted with water (25 mL) and extracted with EtOAc (3×20 mL). The combined organic layers were washed with water, dried over anhydrous Na₂SO₄ and concentrated under reduced pressure. This resulted in 3-(5-fluoro-2-methoxyphenyl)-2-isopropyl-6-methoxy-4-methylpyridine (180 mg, 86%) as a light-yellow solid. m/z: ES⁺ [M+H]⁺=290.15.

### Step 6: 5-(5-fluoro-2-hydroxyphenyl)-6-isopropyl-4-methylpyridin-2(1H)-one

A solution of 3-(5-fluoro-2-methoxyphenyl)-2-isopropyl-6-methoxy-4-methylpyridine (180 mg, 0.62 mmol, 1.0 equiv) and BBr₃ (468 mg, 1.9 mmol, 3.0 equiv) in DCM (3 mL) was stirred at room temperature for 2 h under nitrogen. The reaction was quenched by the addition MeOH at 0° C., and concentrated under reduced pressure. This resulted in 5-(5-fluoro-2-hydroxyphenyl)-6-isopropyl-4-methylpyridin-2(1H)-one (120 mg, 74%) as a light-yellow solid. m/z: ES⁺ [M+H]⁺=262.12.

### 4-fluoro-2-(1-isopropyl-4,5-dimethyl-1H-imidazol-2-yl)phenol

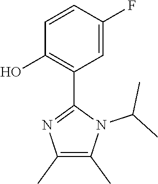 [SMILES: Cc1nc(-c2cc(F)ccc2O)n(C(C)C)c1C]

### Step 1: 2-(2-(benzyloxy)-5-fluorophenyl)-1-isopropyl-1H-imidazole

A mixture of (2-(benzyloxy)-5-fluorophenyl)boronic acid (13.7 g, 55.5 mmol, 1.5 equiv), 2-bromo-1-isopropyl-1H-imidazole (7.0 g, 37 mmol, 1.0 equiv), Pd(dppf)Cl₂ (2.7 g, 3.7 mmol, 0.1 equiv) and K₂CO₃ (10.2 g, 74 mmol, 2.0 equiv) in dioxane (50 mL)/water (13 mL) was stirred at 100° C. for 4 h under nitrogen. The resulting mixture was diluted with water (50 mL) and extracted with EtOAc (3×200 mL). The combined organic layers were washed with brine (100 mL), dried over anhydrous Na₂SO₄, and concentrated under reduced pressure. The residue was purified by silica gel column chromatography, eluting with PE/EtOAc (1:1) to afford 2-(2-(benzyloxy)-5-fluorophenyl)-1-isopropyl-1H-imidazole (5.0 g, 44%) as a light-yellow oil. m/z: ES⁺ [M+H]⁺=311.20.

### Step 2: 2-(2-(benzyloxy)-5-fluorophenyl)-4,5-dibromo-1-isopropyl-1H-imidazole

A mixture of 2-(2-(benzyloxy)-5-fluorophenyl)-1-isopropyl-1H-imidazole (3.0 g, 9.7 mmol, 1.0 equiv) and NBS (5.2 g, 29 mmol, 3.0 equiv) in DMF (30 mL) was stirred at room temperature for 16 h under nitrogen. The resulting mixture was then diluted with water (100 mL) and extracted with EtOAc (3×100 mL). The combined organic layers were washed with brine (100 mL), dried over anhydrous Na₂SO₄, and concentrated under reduced pressure. The residue was purified using method RPFC1 (70% to 80% gradient in 10 min), resulting in 2-(2-(benzyloxy)-5-fluorophenyl)-4,5-dibromo-1-isopropyl-1H-imidazole (2.8 g, 62%) as a brown oil. m/z: ES⁺ [M+H]⁺=468.95.

### Step 3: 2-(2-(benzyloxy)-5-fluorophenyl)-1-isopropyl-4,5-dimethyl-1H-imidazole

A mixture of 2-(2-(benzyloxy)-5-fluorophenyl)-4,5-dibromo-1-isopropyl-1H-imidazole (1.5 g, 3.2 mmol, 1.0 equiv), methylboronic acid (1.2 g, 19 mmol, 6.0 equiv), tBuONa (920 mg, 9.6 mmol, 3.0 equiv), XPhos (230 mg, 0.48 mmol, 0.15 equiv) and Pd₂(dba)₃ (290 mg, 0.32 mmol, 0.1 equiv) in dioxane (15 mL) was stirred at 100° C. for 16 h under nitrogen. The resulting mixture was diluted with water (50 mL) and extracted with EtOAc (3×50 mL). The combined organic layers were washed with brine (2×100 mL), dried over anhydrous Na₂SO₄, and concentrated under reduced pressure. The residue was purified using method RPFC1 (70% to 80% gradient in 10 min), resulting in 2-(2-(benzyloxy)-5-fluorophenyl)-1-isopropyl-4,5-dimethyl-1H-imidazole (400 mg, 37%) as a light-yellow oil. m/z: ES⁺ [M+H]⁺=339.15.

### Step 4: 4-fluoro-2-(1-isopropyl-4,5-dimethyl-1H-imidazol-2-yl)phenol

A mixture of 2-(2-(benzyloxy)-5-fluorophenyl)-1-isopropyl-4,5-dimethyl-1H-imidazole (350 mg, 1.0 mmol, 1.0 equiv) and Pd/C (10% wt, 100 mg, wet) in MeOH (3 mL) was stirred at room temperature for 16 h under hydrogen. The resulting mixture was filtered, the filter cake was washed with MeOH (3 mL) and the filtrate was concentrated under reduced pressure to afford 4-fluoro-2-(1-isopropyl-4,5-dimethyl-1H-imidazol-2-yl)phenol (150 mg, 58%) as a light-yellow oil. m/z: ES⁺ [M+H]⁺=249.15.

### 4-fluoro-2-(5-isopropyl-1-methyl-1H-imidazol-4-yl)phenol

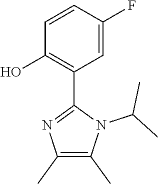 [SMILES: Cc1nc(-c2cc(F)ccc2O)n(C(C)C)c1C]

### Step 1: 2-(2-(benzyloxy)-5-fluorophenyl)-1-isopropyl-1H-imidazole

A mixture of (2-(benzyloxy)-5-fluorophenyl)boronic acid (3.7 g, 15 mmol, 1.2 equiv), 4-bromo-1-methyl-1H-imidazole (2.0 g, 12 mmol, 1.0 equiv), Pd(dppf)Cl₂CH₂Cl₂ (2.0 g, 2.5 mmol, 0.2 equiv) and K₂CO₃ (5.2 g, 37 mmol, 3.0 equiv) in dioxane (20 mL)/water (4 mL) was stirred at 100° C. for 16 h under nitrogen. The resulting mixture was diluted with water (50 mL) and extracted with EtOAc (2×50 mL). The combined organic layers were washed with brine (2×50 mL), dried over anhydrous Na₂SO₄, and concentrated under reduced pressure. The residue was purified using method RPFC1 (10% to 100% gradient in 20 min), resulting in 2-(2-(benzyloxy)-5-fluorophenyl)-1-isopropyl-1H-imidazole (2.4 g, 68%) as a brown solid. m/z: ES⁺ [M+H]⁺=283.15.

### Step 2: 4-(2-(benzyloxy)-5-fluorophenyl)-5-bromo-1-methyl-1H-imidazole

A mixture of 2-(2-(benzyloxy)-5-fluorophenyl)-1-isopropyl-1H-imidazole (1.0 g, 3.5 mmol, 1.0 equiv) and NBS (819 mg, 4.6 mmol, 1.3 equiv) in DMF (10 mL) was stirred at room temperature for 4 h under nitrogen. The resulting mixture was then diluted with water (50 mL) and extracted with EtOAc (2×50 mL). The combined organic layers were washed with brine (2×50 mL), dried over anhydrous Na₂SO₄, and concentrated under reduced pressure. The residue was purified using method RPFC1 (10% to 100% gradient in 20 min), resulting in 4-(2-(benzyloxy)-5-fluorophenyl)-5-bromo-1-methyl-1H-imidazole (540 mg, 46%) as a dark solid. m/z: ES⁺ [M+H]⁺=361.25.

### Step 3: 4-(2-(benzyloxy)-5-fluorophenyl)-1-methyl-5-(prop-1-en-2-yl)-1H-imidazole

A mixture of 4-(2-(benzyloxy)-5-fluorophenyl)-5-bromo-1-methyl-1H-imidazole (590 mg, 1.6 mmol, 1.0 equiv), 4,4,5,5-tetramethyl-2-(prop-1-en-2-yl)-1,3,2-dioxaborolane (548 mg, 3.3 mmol, 2.0 equiv), Pd(dppf)Cl₂CH₂Cl₂ (266 mg, 0.33 mmol, 0.2 equiv), and K₂CO₃ (677 mg, 4.9 mmol, 3.0 equiv) in dioxane (10 mL)/water (2 mL) was stirred at 100° C. for 16 h under nitrogen. The resulting mixture was then diluted with water (50 mL) and extracted with EtOAc (2×50 mL). The combined organic layers were washed with brine (2×50 mL), dried over anhydrous Na₂SO₄, and concentrated under reduced pressure. The residue was purified using method RPFC1 (10% to 100% gradient in 20 min), resulting in 4-(2-(benzyloxy)-5-fluorophenyl)-1-methyl-5-(prop-1-en-2-yl)-1H-imidazole (350 mg, 66%) as a light brown solid. m/z: ES⁺ [M+H]⁺=323.15.

### Step 4: 4-fluoro-2-(5-isopropyl-1-methyl-1H-imidazol-4-yl)phenol

A mixture of 4-(2-(benzyloxy)-5-fluorophenyl)-1-methyl-5-(prop-1-en-2-yl)-1H-imidazole (350 mg, 1.1 mmol, 1.0 equiv) and Pd/C (10% wt, 116 mg, wet) in MeOH (10 mL) was stirred at room temperature for 5 h under hydrogen. The resulting mixture was filtered through Celite and concentrated under reduced pressure to afford 4-fluoro-2-(5-isopropyl-1-methyl-1H-imidazol-4-yl)phenol (170 mg, 56%) as a brown solid. m/z: ES⁺ [M+H]⁺=235.05.

### N-ethyl-N-(5-fluoro-2-hydroxybenzyl)acetamide

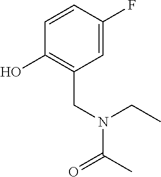

### Step 1: N-ethyl-N-(5-fluoro-2-methoxybenzyl)acetamide

A mixture of N-(5-fluoro-2-methoxybenzyl)ethanamine (780 mg, 4.3 mmol, 1.0 equiv) and acetic anhydride (1.2 mL, 13 mmol, 3.0 equiv), NEt₃ (1.8 mL, 13 mmol, 3.0 equiv) in DCM (6 mL) was stirred at room temperature for 2 h. The resulting mixture was washed with water (3×10 mL) and concentrated under reduced pressure. This resulted in N-ethyl-N-(5-fluoro-2-methoxybenzyl)acetamide (900 mg, 94%) as a yellow oil. m/z: ES⁺ [M+H]⁺=226.00.

### Step 2: N-ethyl-N-(5-fluoro-2-hydroxybenzyl)acetamide

A solution of N-ethyl-N-(5-fluoro-2-methoxybenzyl)acetamide (1.0 g, 4.4 mmol, 1.0 equiv) and BBr₃ (1 M in DCM, 8 mL, 8.0 mmol, 1.8 equiv) in DCM (2 mL) was stirred at room temperature for 1 h under nitrogen. The reaction was quenched by the addition of MeOH at 0° C. The resulting mixture was then diluted with water (20 mL) and extracted with EtOAc (3×10 mL). The combined organic layers were dried over anhydrous Na₂SO₄ and concentrated under reduced pressure. The residue was purified using method RPFC1 (10% to 100% gradient in 20 min), resulting in N-ethyl-N-(5-fluoro-2-hydroxybenzyl)acetamide (500 mg, 53%) as a colourless solid. m/z: ES⁺ [M+H]⁺=212.00.

### N-(5-fluoro-2-hydroxybenzyl)-N-isopropylacetamide

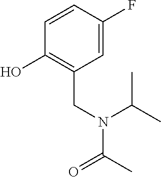 [SMILES: CC(=O)N(Cc1cc(F)ccc1O)C(C)C]

### Step 1: N-(5-fluoro-2-methoxybenzyl)-N-isopropylacetamide

A mixture of N-(5-fluoro-2-methoxybenzyl)propan-2-amine (1.0 g, 5.1 mmol, 1.0 equiv) and acetic anhydride (1.6 g, 15 mmol, 3.0 equiv), NEt₃ (1.5 g, 15 mmol, 3.0 equiv) in DCM (20 mL) was stirred at room temperature for 2 h. The resulting mixture was washed with water (50 mL) and extracted with DCM (2×50 mL). The combined organic layers were washed with water (2×50 mL), dried over anhydrous Na₂SO₄, and concentrated under reduced pressure. This resulted in N-(5-fluoro-2-methoxybenzyl)-N-isopropylacetamide (700 mg, 58%) as a yellow oil. m/z: ES⁺ [M+H]⁺=240.15.

### Step 2: N-(5-fluoro-2-hydroxybenzyl)-N-isopropylacetamide

A solution of N-(5-fluoro-2-methoxybenzyl)-N-isopropylacetamide (750 mg, 3.1 mmol, 1.0 equiv) and BBr₃ (1.6 g, 6.3 mmol, 2.0 equiv) in DCM (20 mL) was stirred at room temperature for 3 h under nitrogen. The reaction was quenched by the addition of MeOH (5 mL) at 0° C. and the resulting mixture was filtered and concentrated under reduced pressure. The residue was purified using method RPFC1 (10% to 100% gradient in 20 min), resulting in N-(5-fluoro-2-hydroxybenzyl)-N-isopropylacetamide (450 mg, 64%) as an off-white solid. m/z: ES⁺ [M+H]⁺=226.15.

### 4-fluoro-2-(5-isopropyl-1,2-dimethyl-1H-imidazol-4-yl)phenol

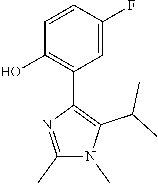 [SMILES: Cc1nc(-c2cc(F)ccc2O)c(C(C)C)n1C]

### Step 1: 4-(2-(benzyloxy)-5-fluorophenyl)-1,2-dimethyl-1H-imidazole

A mixture of (2-(benzyloxy)-5-fluorophenyl)boronic acid (8.4 g, 34 mmol, 3.0 equiv), 4-bromo-1,2-dimethyl-1H-imidazole (2.0 g, 11 mmol, 1.0 equiv), Pd(dppf)Cl₂CH₂Cl₂ (930 mg, 1.1 mmol, 0.1 equiv) and K₂CO₃ (3.2 g, 23 mmol, 2.0 equiv) in dioxane (20 mL)/water (2 mL) was stirred at 100° C. for 2 h under nitrogen. The resulting mixture was diluted with water (10 mL) and extracted with EtOAc (3×10 mL). The combined organic layers were dried over anhydrous Na₂SO₄ and concentrated under reduced pressure. The residue was purified using method RPFC2 (10% to 100% gradient in 20 min), resulting in 4-(2-(benzyloxy)-5-fluorophenyl)-1,2-dimethyl-1H-imidazole (1.5 g, 44%) as a brown solid. m/z: ES⁺ [M+H]⁺=297.13.

### Step 2: 4-(2-(benzyloxy)-5-fluorophenyl)-5-bromo-1,2-dimethyl-1H-imidazole

A mixture of 4-(2-(benzyloxy)-5-fluorophenyl)-1,2-dimethyl-1H-imidazole (1.5 g, 5.1 mmol, 1.0 equiv) and NBS (901 mg, 5.1 mmol, 1.0 equiv) in MeCN (20 mL) was stirred at room temperature for 1 h under nitrogen. The resulting mixture was then diluted with water (30 mL) and extracted with DCM (3×10 mL). The combined organic layers were dried over anhydrous Na₂SO₄ and concentrated under reduced pressure to give 4-(2-(benzyloxy)-5-fluorophenyl)-5-bromo-1,2-dimethyl-1H-imidazole (1.8 g, 95%) as a brown solid. m/z: ES⁺ [M+H]⁺=375.04.

### Step 3: 4-(2-(benzyloxy)-5-fluorophenyl)-1,2-dimethyl-5-(prop-1-en-2-yl)-1H-imidazole

A mixture of 4-(2-(benzyloxy)-5-fluorophenyl)-5-bromo-1,2-dimethyl-1H-imidazole (1.8 g, 4.8 mmol, 1.0 equiv), 4,4,5,5-tetramethyl-2-(prop-1-en-2-yl)-1,3,2-dioxaborolane (2.4 g, 14 mmol, 3.0 equiv), Pd(PPh₃)₄ (550 mg, 0.48 mmol, 0.1 equiv) and K₃PO₄ (2.0 g, 9.6 mmol, 2.0 equiv) in dioxane (18 mL)/water (3 mL) was stirred at 100° C. for 4 h under nitrogen. The mixture was diluted with water (10 mL) and extracted with DCM (3×10 mL). The combined organic layers were dried over anhydrous Na₂SO₄ and concentrated under reduced pressure. The residue was purified using method RPFC2 (0% to 80% gradient in 20 min), resulting in 4-(2-(benzyloxy)-5-fluorophenyl)-1,2-dimethyl-5-(prop-1-en-2-yl)-1H-imidazole (1.1 g, 68%) as a yellow solid. m/z: ES⁺ [M+H]⁺=337.16.

### Step 4: 4-fluoro-2-(5-isopropyl-1,2-dimethyl-1H-imidazol-4-yl)phenol

A suspension of 4-(2-(benzyloxy)-5-fluorophenyl)-1,2-dimethyl-5-(prop-1-en-2-yl)-1H-imidazole (220 mg, 0.65 mmol, 1.0 equiv) and Pd/C (10% wt, wet, 70 mg) in MeOH (5 mL) was stirred at room temperature for 4 h under hydrogen. The resulting mixture was filtered, the filter cake was washed with MeOH (3×5 mL) and the filtrate was concentrated under reduced pressure to give 4-fluoro-2-(5-isopropyl-1,2-dimethyl-1H-imidazol-4-yl)phenol (150 mg, 92%) as a colourless solid. m/z: ES⁺ [M+H]⁺=249.13.

### 4-fluoro-2-(1-isopropyl-2-methyl-1H-imidazol-4-yl)phenol

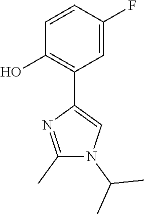 [SMILES: Cc1nc(-c2cc(F)ccc2O)cn1C(C)C]

A suspension of 4-(2-(benzyloxy)-5-fluorophenyl)-1-isopropyl-2-methyl-1H-imidazole (300 mg, 0.92 mmol, 1.0 equiv) and Pd/C (10% wt, wet, 196 mg) in MeOH (3 mL) was stirred at room temperature for 2 h under hydrogen. The resulting mixture was filtered, the filter cake was washed with MeOH (3×10 mL) and the filtrate was concentrated under reduced pressure to give 4-fluoro-2-(1-isopropyl-2-methyl-1H-imidazol-4-yl)phenol (120 mg, 55%) as a brown oil. m/z: ES⁺ [M+H]⁺=235.05.

### tert-butyl (S)-2-((1S,5R)-2,6-diazabicyclo[3.2.1]octane-2-carbonyl)-2-methylpyrrolidine-1-carboxylate

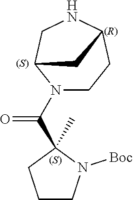

### Step 1: benzyl (1S,5R)-2-[(2S)-1-(tert-butoxycarbonyl)-2-methylpyrrolidine-2-carbonyl]-2,6-diazabicyclo[3.2.1]octane-6-carboxylate

The amide bond coupling between benzyl (1S,5R)-2,6-diazabicyclo[3.2.1]octane-6-carboxylate (8.0 g, 32 mmol, 1.0 equiv) and (S)-1-(tert-butoxycarbonyl)-2-methylpyrrolidine-2-carboxylic acid (6.0 g, 26 mmol, 0.8 equiv) was performed according to general procedure A1. The resulting mixture was diluted with water (100 mL), and was extracted with DCM (3×50 mL). The combined organic layers were washed with brine (100 mL), dried over Na₂SO₄. After filtration, the filtrate was concentrated under reduced pressure, and the residue was purified using method RPCF1 (70% to 80% gradient in 10 min). This resulted in benzyl (1S,5R)-2-[(2S)-1-(tert-butoxycarbonyl)-2-methylpyrrolidine-2-carbonyl]-2,6-diazabicyclo[3.2.1]octane-6-carboxylate (10 g, 67%) as a light yellow oil. m/z: ES⁺ [M+H]⁺=458.30.

### Step 2: tert-butyl (2S)-2-[(1S,5R)-2,6-diazabicyclo[3.2.1]octane-2-carbonyl]-2-methylpyrrolidine-1-carboxylate

To a solution of benzyl (1S,5R)-2-[(2S)-1-(tert-butoxycarbonyl)-2-methylpyrrolidine-2-carbonyl]-2,6-diazabicyclo[3.2.1]octane-6-carboxylate (10.0 g, 22 mmol, 1.0 equiv) in MeOH (100 mL), was added Pd/C (10%, 0.23 g, 2.2 mmol, 0.1 equiv) in a pressure tank. The mixture was stirred at room temperature under hydrogen (10 PSI) overnight, and then filtered through a Celite pad and concentrated under reduced pressure. The resulting mixture was filtered, the filter cake was washed with MeOH (3×100 mL), and the filtrate was concentrated under reduced pressure. This resulted in tert-butyl (2S)-2-[(1S,5R)-2,6-diazabicyclo[3.2.1]octane-2-carbonyl]-2-methylpyrrolidine-1-carboxylate (8.5 g, crude) as a light yellow oil. m/z: ES⁺ [M+H]⁺=324.15.

### 4-chloro-5-(2-(2-cyclopropyl-1H-imidazol-1-yl)-4-fluorophenoxy)pyrimidine

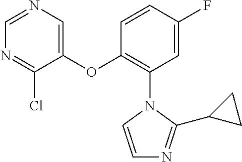 [SMILES: Fc1ccc(Oc2cncnc2Cl)c(-n2ccnc2C2CC2)c1]

The title 4-chloropyrimidine was prepared according to general procedure E1.

### Step 1: ethyl 2-(2-(2-cyclopropyl-1H-imidazol-1-yl)-4-fluorophenoxy)acetate

Purified using method RPC1 (20% to 80% gradient in 30 min) to afford ethyl 2-(2-(2-cyclopropyl-1H-imidazol-1-yl)-4-fluorophenoxy)acetate (1.8 g, 51%) as alight yellow oil. m/z: ES⁺ [M+H]⁺=304.95.

### Step 2: ethyl (Z)-2-(2-(2-cyclopropyl-1H-imidazol-1-yl)-4-fluorophenoxy)-3-hydroxyacrylate

The crude product was used in the next step without further purification. m/z: ES⁺ [M+H]⁺=332.95.

### Step 3: 5-(2-(2-cyclopropyl-1H-imidazol-1-yl)-4-fluorophenoxy)-2-thioxo-2,3-dihydropyrimidin-4(1H)-one

Purified using method RPC1 (0% to 40% gradient in 40 min) to afford 5-(2-(2-cyclopropyl-1H-imidazol-1-yl)-4-fluorophenoxy)-2-thioxo-2,3-dihydropyrimidin-4(1H)-one (1.0 g, 53%) as a light brown oil. m/z: ES⁺ [M+H]⁺=345.20.

### Step 4: 5-(2-(2-cyclopropyl-1H-imidazol-1-yl)-4-fluorophenoxy)pyrimidin-4-ol

The crude product was obtained as a yellow oil (700 mg, quant.), and was used in the next step without further purification. m/z: ES⁺ [M+H]⁺=313.00.

### Step 5: 4-chloro-5-(2-(2-cyclopropyl-1H-imidazol-1-yl)-4-fluorophenoxy)pyrimidine

The crude product was obtained as a yellow oil (250 mg, quant.), and was used in the next step without further purification. m/z: ES⁺ [M+H]⁺=330.95.

### 4-chloro-5-(2-(2-cyclopropyl-5-methyl-1H-imidazol-1-yl)-4-fluorophenoxy)pyrimidine

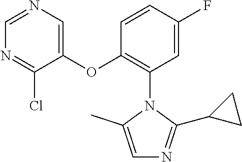 [SMILES: Cc1cnc(C2CC2)n1-c1cc(F)ccc1Oc1cncnc1Cl]

The title 4-chloropyrimidine was prepared according to general procedure E1.

### Step 1: ethyl 2-(2-(2-cyclopropyl-5-methyl-1H-imidazol-1-yl)-4-fluorophenoxy)acetate

Purified using method RPFC4 (0% to 100% gradient in 30 min) to afford ethyl 2-(2-(2-cyclopropyl-5-methyl-1H-imidazol-1-yl)-4-fluorophenoxy)acetate (4.0 g, 49%) as a yellow solid. m/z: ES⁺ [M+H]⁺=319.15.

### Step 2: ethyl (Z)-2-(2-(2-cyclopropyl-5-methyl-1H-imidazol-1-yl)-4-fluorophenoxy)-3-hydroxyacrylate

The crude product was obtained as a black oil (5 g), which was used in the next step without further purification. m/z: ES⁺ [M+H]⁺=347.00.

### Step 3: 5-(2-(2-cyclopropyl-5-methyl-1H-imidazol-1-yl)-4-fluorophenoxy)-2-thioxo-2,3-dihydropyrimidin-4(1H)-one

Purified using method RPFC4 (10% to 80% gradient in 15 min) to afford 5-(2-(2-cyclopropyl-5-methyl-1H-imidazol-1-yl)-4-fluorophenoxy)-2-thioxo-2,3-dihydropyrimidin-4(1H)-one (2.0 g, 48%) as a colourless solid. m/z: ES⁺ [M+H]⁺=358.95.

### Step 4: 5-(2-(2-cyclopropyl-5-methyl-1H-imidazol-1-yl)-4-fluorophenoxy)pyrimidin-4-ol

The crude product was obtained as a yellow solid (850 mg, 93%), and was used in the next step without further purification. m/z: ES⁺ [M+H]⁺=327.20.

### Step 5: 4-chloro-5-(2-(2-cyclopropyl-5-methyl-1H-imidazol-1-yl)-4-fluorophenoxy)pyrimidine

Purified by Prep-TLC (DCM/MeOH, 20:1) to afford 4-chloro-5-(2-(2-cyclopropyl-5-methyl-1H-imidazol-1-yl)-4-fluorophenoxy)pyrimidine (400 mg, 47%) as a colourless solid. m/z: ES⁺ [M+H]⁺=345.10.

### 4-chloro-5-(2-(2-isopropyl-1H-imidazol-1-yl)-4-fluorophenoxy)pyrimidine

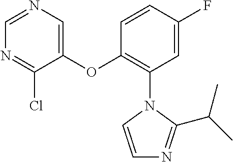 [SMILES: CC(C)c1nccn1-c1cc(F)ccc1Oc1cncnc1Cl]

The title 4-chloropyrimidine was prepared according to general procedure E1.

### Step 1: ethyl 2-(2-(2-isopropyl-1H-imidazol-1-yl)-4-fluorophenoxy)acetate

Purified using method RPC1 (10% to 100% gradient in 40 min) to afford ethyl 2-(2-(2-isopropyl-1H-imidazol-1-yl)-4-fluorophenoxy)acetate (5.0 g, 72%) as a yellow oil. m/z: ES⁺ [M+H]⁺=307.10.

### Step 2: ethyl (Z)-2-(2-(2-isopropyl-1H-imidazol-1-yl)-4-fluorophenoxy)-3-hydroxyacrylate

The crude product was obtained as a brown oil (4.0 g, quant.), and used in the next step without further purification. m/z: ES⁺ [M+H]⁺=335.05.

### Step 3: 5-(2-(2-isopropyl-1H-imidazol-1-yl)-4-fluorophenoxy)-2-thioxo-2,3-dihydropyrimidin-4(1H)-one

Purified using method RPC1 (10% to 50% gradient in 40 min) to afford 5-(2-(2-isopropyl-1H-imidazol-1-yl)-4-fluorophenoxy)-2-thioxo-2,3-dihydropyrimidin-4(1H)-one (2.0 g, 48%) as an off-white solid. m/z: ES⁺ [M+H]⁺=347.05.

### Step 4: 5-(2-(2-isopropyl-1H-imidazol-1-yl)-4-fluorophenoxy)pyrimidin-4-ol

The crude product was obtained as an off-white solid (600 mg, 55%), and was used in the next step without further purification. m/z: ES⁺ [M+H]⁺=315.05.

### Step 5: 4-chloro-5-(2-(2-isopropyl-1H-imidazol-1-yl)-4-fluorophenoxy)pyrimidine

The crude product was obtained as a yellow oil (100 mg, quant.), and was used in the next step without further purification. m/z: ES⁺ [M+H]⁺=333.00.

### 4-chloro-5-(2-(2,5-dicyclopropyl-1H-imidazol-1-yl)-4-fluorophenoxy)pyrimidine

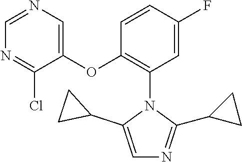 [SMILES: Fc1ccc(Oc2cncnc2Cl)c(-n2c(C3CC3)cnc2C2CC2)c1]

The title 4-chloropyrimidine was prepared according to general procedure E1.

### Step 1: ethyl 2-(2-(2,5-dicyclopropyl-1H-imidazol-1-yl)-4-fluorophenoxy)acetate

Purified using method RPFC1 (10% to 100% gradient in 20 min) to afford ethyl 2-(2-(2-cyclopropyl-5-methyl-1H-imidazol-1-yl)-4-fluorophenoxy)acetate (850 mg, 32%) as a brown solid. m/z: ES⁺ [M+H]⁺=345.05.

### Step 2: ethyl (Z)-2-(2-(2,5-dicyclopropyl-1H-imidazol-1-yl)-4-fluorophenoxy)-3-hydroxyacrylate

The crude product was obtained as a brown oil (800 mg g), which was used in the next step without further purification. m/z: ES⁺ [M+H]⁺=373.00.

### Step 3: 5-(2-(2,5-dicyclopropyl-1H-imidazol-1-yl)-4-fluorophenoxy)-2-thioxo-2,3-dihydropyrimidin-4(1H)-one

Purified using method RPFC1 (10% to 100% gradient in 20 min) to afford 5-(2-(2,5-dicyclopropyl-1H-imidazol-1-yl)-4-fluorophenoxy)-2-thioxo-2,3-dihydropyrimidin-4(1H)-one (500 mg, 61%) as a brown oil. m/z: ES⁺ [M+H]⁺=385.10.

### Step 4: 5-(2-(2,5-dicyclopropyl-1H-imidazol-1-yl)-4-fluorophenoxy)pyrimidin-4-ol

The crude product was obtained as a brown oil (260 mg, 57%), and was used in the next step without further purification. m/z: ES⁺ [M+H]⁺=353.05.

### Step 5: 4-chloro-5-(2-(2,5-dicyclopropyl-1H-imidazol-1-yl)-4-fluorophenoxy)pyrimidine

The crude product was obtained as a brown oil (240 mg, 89%), and was used in the next step without further purification. m/z: ES⁺ [M+H]⁺=371.00.

### 4-chloro-5-(2-(2-cyclopropyl-5-methyl-1H-imidazol-1-yl)-4-fluorophenoxy)pyrimidine

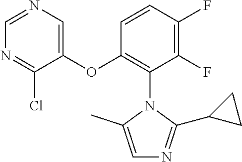 [SMILES: Cc1cnc(C2CC2)n1-c1c(Oc2cncnc2Cl)ccc(F)c1F]

The title 4-chloropyrimidine was prepared according to general procedure E1.

### Step 1: ethyl 2-(2-(2-cyclopropyl-5-methyl-1H-imidazol-1-yl)-3,4-difluorophenoxy)acetate

Purified using method RPCF4 (0% to 100% gradient in 25 min) to afford ethyl 2-(2-(2-cyclopropyl-5-methyl-1H-imidazol-1-yl)-3,4-difluorophenoxy)acetate (1.5 g, 59%) as a yellow oil. m/z: ES⁺ [M+H]⁺=337.36.

### Step 2: ethyl (Z)-2-(2-(2-cyclopropyl-5-methyl-1H-imidazol-1-yl)-3,4-difluorophenoxy)-3-hydroxyacrylate

The crude product was used in the next step without further purification. m/z: ES⁺ [M+H]⁺=365.18.

### Step 3: 5-(2-(2-cyclopropyl-5-methyl-1H-imidazol-1-yl)-3,4-difluorophenoxy)-2-thioxo-2,3-dihydropyrimidin-4(1H)-one

Purified using method RPCF3 (0% to 100% gradient in 25 min) to afford 5-(2-(2-cyclopropyl-5-methyl-1H-imidazol-1-yl)-3,4-difluorophenoxy)-2-thioxo-2,3-dihydropyrimidin-4(1H)-one (560 mg, 49%) as a yellow oil. m/z: ES⁺ [M+H]⁺=377.15.

### Step 4: 5-(2-(2-cyclopropyl-5-methyl-1H-imidazol-1-yl)-3,4-difluorophenoxy)pyrimidin-4-ol

The crude product was used in the next step without further purification. m/z: ES⁺ [M+H]⁺=345.32.

### Step 5: 4-chloro-5-(2-(2-cyclopropyl-5-methyl-1H-imidazol-1-yl)-3,4-difluorophenoxy)pyrimidine

Purified by Prep-TLC (PE/EtOAc 1:1) to afford 4-chloro-5-(2-(2-cyclopropyl-5-methyl-1H-imidazol-1-yl)-3,4-difluorophenoxy)pyrimidine (250 mg, 48%) as a yellow oil. m/z: ES⁺ [M+H]⁺=363.76.

### 4-chloro-5-(4-fluoro-2-(2-isopropyl-5-methyl-1H-imidazol-1-yl)phenoxy)pyrimidine

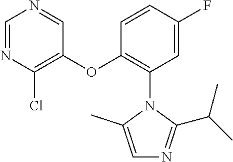 [SMILES: Cc1cnc(C(C)C)n1-c1cc(F)ccc1Oc1cncnc1Cl]

The title 4-chloropyrimidine was prepared according to general procedure E1.

### Step 1: ethyl 2-(4-fluoro-2-(2-isopropyl-5-methyl-1H-imidazol-1-yl)phenoxy)acetate

The crude product was purified using method RPFC1 (10% to 100% gradient in 20 min) to afford ethyl 2-(4-fluoro-2-(2-isopropyl-5-methyl-1H-imidazol-1-yl) phenoxy) acetate (6.0 g, 44%) as a brown solid. m/z: ES⁺ [M+H]⁺=321.20.

### Step 2: ethyl (Z)-2-(4-fluoro-2-(2-isopropyl-5-methyl-1H-imidazol-1-yl)phenoxy)-3-hydroxyacrylate

The crude product (7.0 g) was used in the next step without further purification. m/z: ES⁺ [M+H]⁺=349.25.

### Step 3: 5-(4-fluoro-2-(2-isopropyl-5-methyl-1H-imidazol-1-yl)phenoxy)-2-thioxo-2,3-dihydropyrimidin-4(1H)-one

The product (2.8 g, 39%) was obtained as an off-white solid, and was used in the next step without further purification. m/z: ES⁺ [M+H]⁺=361.05.

### Step 4: 5-(4-fluoro-2-(2-isopropyl-5-methyl-1H-imidazol-1-yl)phenoxy)pyrimidin-4-ol

The product (1.5 g, 61%) was obtained as an off-white solid, and was used in the next step without further purification. m/z: ES⁺ [M+H]⁺=329.20.

### Step 5: 4-chloro-5-(4-fluoro-2-(2-isopropyl-5-methyl-1H-imidazol-1-yl)phenoxy)pyrimidine

The crude product was purified by silica gel column chromatography (PE/EtOAC; 1:1) to afford 4-chloro-5-(4-fluoro-2-(2-isopropyl-5-methyl-1H-imidazol-1-yl)phenoxy)pyrimidine (1.4 g, 88%) as a light yellow solid. m/z: ES⁺ [M+H]⁺=347.20.

### 4-chloro-5-(2-(3,5-dicyclopropyl-4H-1,2,4-triazol-4-yl)-4-fluorophenoxy)pyrimidine

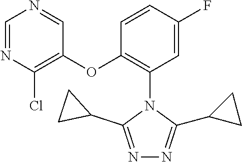 [SMILES: Fc1ccc(Oc2cncnc2Cl)c(-n2c(C3CC3)nnc2C2CC2)c1]

The title 4-chloropyrimidine was prepared according to general procedure E1.

### Step 1: ethyl 2-(2-(3,5-dicyclopropyl-4H-1,2,4-triazol-4-yl)-4-fluorophenoxy)acetate

Purified using method RPFC1 (10% to 100% gradient in 20 min) to afford 2-(2-(3,5-dicyclopropyl-4H-1,2,4-triazol-4-yl)-4-fluorophenoxy)acetate (17 g, 71%) as a yellow solid. m/z: ES⁺ [M+H]⁺=346.00.

### Step 2: ethyl (Z)-2-(2-(3,5-dicyclopropyl-4H-1,2,4-triazol-4-yl)-4-fluorophenoxy)-3-hydroxyacrylate

The crude product was used in the next step without further purification. m/z: ES⁺ [M+H]⁺=374.10.

### Step 3: 5-(2-(3,5-dicyclopropyl-4H-1,2,4-triazol-4-yl)-4-fluorophenoxy)-2-thioxo-2,3-dihydropyrimidin-4(1H)-one

Purified using method RPFC1 (5% to 50% gradient in 20 min) to afford 5-(2-(3,5-dicyclopropyl-4H-1,2,4-triazol-4-yl)-4-fluorophenoxy)-2-thioxo-2,3-dihydropyrimidin-4(1H)-one (11 g, 58%) as a yellow solid. m/z: ES⁺ [M+H]⁺=385.95.

### Step 4: 5-(2-(3,5-dicyclopropyl-4H-1,2,4-triazol-4-yl)-4-fluorophenoxy)pyrimidin-4-ol

The crude product (9.0 g, 89%) was obtained as a yellow solid, and was used in the next step without further purification. m/z: ES⁺ [M+H]⁺=354.00.

### Step 5: 4-chloro-5-(2-(3,5-dicyclopropyl-4H-1,2,4-triazol-4-yl)-4-fluorophenoxy)pyrimidine

The crude product (7.0 g, 74%) was obtained as a yellow solid, and was used in the next step without further purification. m/z: ES⁺ [M+H]⁺=372.05.

### 6-(2-((4-chloropyrimidin-5-yl)oxy)-5-fluorophenyl)-5-isopropyl-1-methylpyridin-2(1H)-one

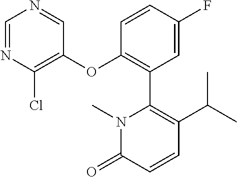 [SMILES: CC(C)c1ccc(=O)n(C)c1-c1cc(F)ccc1Oc1cncnc1Cl]

### Step 1: 6-(5-fluoro-2-(pyrimidin-5-yloxy)phenyl)-5-isopropyl-1-methylpyridin-2(1H)-one

To a stirred solution of 6-(5-fluoro-2-hydroxyphenyl)-5-isopropyl-1-methylpyridin-2(1H)-one (2.5 g, 9.6 mmol, 1.0 equiv) and 5-fluoropyrimidine (1.1 g, 12 mmol, 1.2 equiv) in DMF (25 mL) was added Cs₂CO₃ (6.2 g, 19 mmol, 2.0 equiv) and the resulting mixture was stirred at 100° C. for 16 h. It was then diluted with water (200 mL), and extracted with DCM (2×200 mL). The combined organic layers were washed with brine (2×300 mL), dried over Na₂SO₄, and concentrated under reduced pressure to give a residue which was purified using method RPFC1 (10% to 50% gradient in 10 min) to afford 6-(5-fluoro-2-(pyrimidin-5-yloxy)phenyl)-5-isopropyl-1-methylpyridin-2(1H)-one (2.0 g, 61%) as a brown solid. m/z: ES⁺ [M+H]⁺=340.10.

### Step 2: 5-(4-fluoro-2-(3-isopropyl-1-methyl-6-oxo-1,6-dihydropyridin-2-yl)phenoxy)pyrimidine 1-oxide

A solution of 6-(5-fluoro-2-(pyrimidin-5-yloxy)phenyl)-5-isopropyl-1-methylpyridin-2(1H)-one (2.0 g, 5.9 mmol, 1.0 equiv) and mCPBA (2.5 g, 15 mmol, 2.5 equiv) in DCM (20 mL) was stirred at 40° C. for 16 h. The reaction was quenched by the addition of sat. NaS₂O₃ (150 mL), and extracted with DCM (2×100 mL). The combined organic layers were washed with brine (2×200 mL), dried over Na₂SO₄, filtered and concentrated under reduced pressure to give a residue which was purified using method RPFC1 (10% to 70% gradient in 25 min) to afford 5-(4-fluoro-2-(3-isopropyl-1-methyl-6-oxo-1,6-dihydropyridin-2-yl)phenoxy)pyrimidine 1-oxide (1.1 g, 52%) as a colourless solid. m/z: ES⁺ [M+H]⁺=356.05.

### Step 3: 6-(2-((4-chloropyrimidin-5-yl)oxy)-5-fluorophenyl)-5-isopropyl-1-methylpyridin-2(1H)-one

A solution of 5-(4-fluoro-2-(3-isopropyl-1-methyl-6-oxo-1,6-dihydropyridin-2-yl)phenoxy)pyrimidine 1-oxide (1.1 g, 3.1 mmol, 1.0 equiv) in DCM (10 mL) was treated with NEt₃ (0.94 g, 9.3 mmol, 3.0 equiv) followed by the dropwise addition of POCl₃ (1.4 g, 9.3 mmol, 3.0 equiv) at 0° C. and the resulting mixture was stirred at room temperature for 1 h. The reaction was quenched by the addition of sat. NaHCO₃ (100 mL) at 0° C., and extracted with DCM (2×100 mL). The combined organic layers were washed with brine (2×200 mL), dried over Na₂SO₄, filtered and concentrated under reduced pressure to afford 6-(2-((4-chloropyrimidin-5-yl)oxy)-5-fluorophenyl)-5-isopropyl-1-methyl pyridin-2(1H)-one (400 mg, 34%) as a yellow solid. m/z: ES⁺ [M+H]⁺=374.0. tert-butyl (1S,5R)-6-(3,6-dichloro-1,2,4-triazin-5-yl)-2,6-diazabicyclo[3.2.1]octane-2-carboxylate

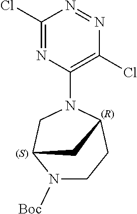

A solution of 3,5,6-trichloro-1,2,4-triazine (1.0 g, 5.4 mmol, 1.0 equiv), tert-butyl (1S,5R)-2,6-diazabicyclo[3.2.1]octane-2-carboxylate (2.3 g, 11 mmol, 2.0 equiv) and Et₃N (1.7 g, 16 mmol, 3.0 equiv) in DCM (10 mL) was stirred at room temperature overnight. The resulting mixture was concentrated under reduced pressure and the residue was purified using method RFPC1 (10% to 90% gradient in 20 min) to give the title triazine (1.3 g, 67%) as a yellow solid. m/z: ES⁺ [M+H]⁺=360.24. tert-butyl (S)-2-((1S,5R)-6-(3,6-dichloro-1,2,4-triazin-5-yl)-2,6-diazabicyclo[3.2.1]octane-2-carbonyl)-2-methylpyrrolidine-1-carboxylate

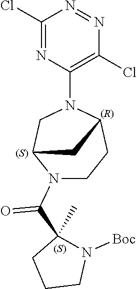

To a stirred solution of trichloro-1,2,4-triazine (2.9 g, 15.5 mmol, 1.0 equiv) and NEt₃ (3.1 g, 31 mmol, 2.0 equiv) in DCM (50 mL) were added tert-butyl (2S)-2-[(1S,5R)-2,6-diazabicyclo[3.2.1]octane-2-carbonyl]-2-methylpyrrolidine-1-carboxylate (5.0 g, 15.5 mmol, 1.0 equiv) dropwise at 0° C. under nitrogen. The resulting mixture was stirred at room temperature for 2 h under nitrogen, and then concentrated under reduced pressure. The residue was purified by silica gel column chromatography, eluted with PE/EtOAc (1:1) to afford the title triazine (2.5 g, 34%) as a light yellow solid. m/z: ES⁺ [M+H]⁺=471.00.

### 2-((5-((1S,5R)-2,6-diazabicyclo[3.2.1]octan-6-yl)-3-chloro-1,2,4-triazin-6-yl)oxy)-N-ethyl-5-fluoro-N-isopropylbenzamide

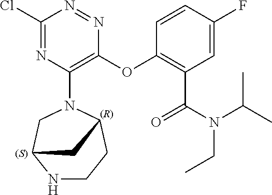

### Step 1: tert-butyl (1S,5R)-6-(3-chloro-6-(2-(ethyl(isopropyl)carbamoyl)-4-fluorophenoxy)-1,2,4-triazin-5-yl)-2,6-diazabicyclo[3.2.1]octane-2-carboxylate

To a stirred solution of N-ethyl-5-fluoro-2-hydroxy-N-isopropylbenzamide (400 mg, 1.8 mmol, 1.0 equiv) and tert-butyl (1S,5R)-6-(3,6-dichloro-1,2,4-triazin-5-yl)-2,6-diazabicyclo[3.2.1]octane-2-carboxylate (638 mg, 1.8 mmol, 1.0 equiv) in THF (1 mL) was added DBU (324 mg, 2.1 mmol, 1.2 equiv) dropwise at room temperature. The resulting mixture was stirred at 25° C. for 3 h then concentrated under vacuum to give a residue which was purified using method RFPC1 (10% to 100% gradient in 10 min) to afford tert-butyl (1S,5R)-6-(3-chloro-6-(2-(ethyl(isopropyl)carbamoyl)-4-fluorophenoxy)-1,2,4-triazin-5-yl)-2,6-diazabicyclo[3.2.1]octane-2-carboxylate (450 mg, 46%) as a yellow solid. (ES, m/z): [M+H]⁺=549.24.

### Step 2: 2-((5-((1S,5R)-2,6-diazabicyclo[3.2.1]octan-6-yl)-3-chloro-1,2,4-triazin-6-yl)oxy)-N-ethyl-5-fluoro-N-isopropylbenzamide

The Boc deprotection of tert-butyl (1S,5R)-6-(3-chloro-6-(2-(ethyl(isopropyl)carbamoyl)-4-fluorophenoxy)-1,2,4-triazin-5-yl)-2,6-diazabicyclo[3.2.1]octane-2-carboxylate (430 mg, 0.8 mmol) was performed according to general procedure B2. The reaction mixture was concentrated under vacuum to give 2-((5-((1S,5R)-2,6-diazabicyclo[3.2.1]octan-6-yl)-3-chloro-1,2,4-triazin-6-yl)oxy)-N-ethyl-5-fluoro-N-isopropyl benzamide (450 mg, 90%) as a yellow solid. (ES, m/z): [M+H]⁺=449.18.

### N-(2-((5-((1S,5R)-2,6-diazabicyclo[3.2.1]octan-6-yl)-3-chloro-1,2,4-triazin-6-yl)oxy)-5-fluorophenyl)-N-methylisobutyramide

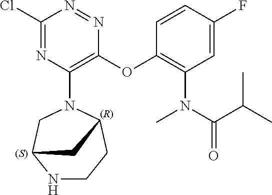

### Step 1: tert-butyl (1S,5R)-6-(3-chloro-6-(2-(N-methylisobutyramido)-4-fluorophenoxy)-1,2,4-triazin-5-yl)-2,6-diazabicyclo[3.2.1]octane-2-carboxylate

tert-Butyl (1S,5R)-6-(3-chloro-6-(2-(N-methylisobutyramido)-4-fluorophenoxy)-1,2,4-triazin-5-yl)-2,6-diazabicyclo[3.2.1]octane-2-carboxylate was synthesized using N-ethyl-N-(5-fluoro-2-hydroxyphenyl)isobutyramide (500 mg, 1.4 mmol, 1.0 equiv) according to general procedure C₁. The resulting mixture was diluted with water (20 mL), and was extracted with EtOAc (3×20 mL). The combined organic layers were washed with brine (10 mL), dried over Na₂SO₄ and concentrated under reduced pressure. The residue was purified by silica gel column chromatography, eluted with PE/EtOAc (1:1) to afford the desired product (500 mg, 67%) as a light yellow oil. m/z: ES⁺ [M+H]⁺=535.40.

### Step 2: N-(2-((5-((1S,5R)-2,6-diazabicyclo[3.2.1]octan-6-yl)-3-chloro-1,2,4-triazin-6-yl)oxy)-5-fluorophenyl)-N-methylisobutyramide

The Boc deprotection of tert-butyl (1S,5R)-6-(3-chloro-6-(2-(N-methylisobutyramido)-4-fluorophenoxy)-1,2,4-triazin-5-yl)-2,6-diazabicyclo[3.2.1]octane-2-carboxylate (500 mg, 0.9 mmol, 1.0 equiv) was performed according to general procedure B2. The resulting reaction mixture was concentrated under reduced pressure, affording crude N-(2-((5-((1S,5R)-2,6-diazabicyclo[3.2.1]octan-6-yl)-3-chloro-1,2,4-triazin-6-yl)oxy)-5-fluorophenyl)-N-methylisobutyramide (400 mg, quant.) as a light yellow oil. (ES, m/z): [M+H]⁺=435.25.

### N-(2-((5-((1S,5R)-2,6-diazabicyclo[3.2.1]octan-6-yl)-3-chloro-1,2,4-triazin-6-yl)oxy)-5-fluorophenyl)-N-ethylisobutyramide

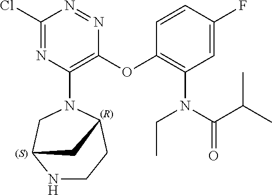

### Step 1: tert-butyl (1S,5R)-6-(3-chloro-6-(2-(N-ethylisobutyramido)-4-fluorophenoxy)-1,2,4-triazin-5-yl)-2,6-diazabicyclo[3.2.1]octane-2-carboxylate

tert-Butyl (1S,5R)-6-(3-chloro-6-(2-(N-ethylisobutyramido)-4-fluorophenoxy)-1,2,4-triazin-5-yl)-2,6-diazabicyclo[3.2.1]octane-2-carboxylate was synthesized using N-ethyl-N-(5-fluoro-2-hydroxyphenyl)isobutyramide (312 mg, 1.4 mmol, 1.0 equiv) according to general procedure C₁. The resulting mixture was diluted with water (20 mL), and was extracted with EtOAc (3×20 mL). The combined organic layers were washed with brine (10 mL), dried over Na₂SO₄ and concentrated under reduced pressure. The residue was purified by silica gel column chromatography, eluted with PE/EtOAc (1:1) to afford the desired product (550 mg, 80%) as a light yellow oil. m/z: ES⁺ [M+H]⁺=549.20.

### Step 2: N-(2-((5-((1S,5R)-2,6-diazabicyclo[3.2.1]octan-6-yl)-3-chloro-1,2,4-triazin-6-yl)oxy)-5-fluorophenyl)-N-ethylisobutyramide

The Boc deprotection of tert-butyl (1S,5R)-6-(3-chloro-6-(2-(N-ethylisobutyramido)-4-fluorophenoxy)-1,2,4-triazin-5-yl)-2,6-diazabicyclo[3.2.1]octane-2-carboxylate (500 mg, 0.91 mmol, 1 equiv) was performed according to general procedure B1. The pH of the crude reaction mixture was adjusted to pH=7 with sat. NaHCO₃ and the resulting mixture was extracted with DCM (3×10 mL). The combined organic layers were washed with brine (10 mL), dried over Na₂SO₄, and concentrated under reduced pressure. The residue was purified using method RFPC4 (40% to 50% gradient in 10 min), affording N-(2-((5-((1S,5R)-2,6-diazabicyclo[3.2.1]octan-6-yl)-3-chloro-1,2,4-triazin-6-yl)oxy)-5-fluorophenyl)-N-ethylisobutyramide (400 mg, 98%) as a light yellow oil. (ES, m/z): [M+H]⁺=449.25.

### tert-butyl (1S,5R)-6-(3-chloro-6-(2-(2-cyclopropyl-1H-imidazol-1-yl)-4-fluorophenoxy)-1,2,4-triazin-5-yl)-2,6-diazabicyclo[3.2.1]octane-2-carboxylate

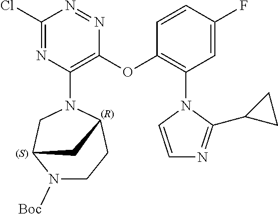

tert-Butyl (1S,5R)-6-(3,6-dichloro-1,2,4-triazin-5-yl)-2,6-diazabicyclo[3.2.1]octane-2-carboxylate (450 mg, 1.25 mmol, 1.0 equiv) and 2-(2-cyclopropyl-1H-imidazol-1-yl)-4-fluorophenol (299 mg, 1.4 mmol, 1.1 equiv) were coupled according to general procedure C₁. The resulting mixture was concentrated under reduced pressure, and the crude product was purified using method RFPC1 (10% to 100% gradient in 20 min) to afford tert-butyl (1S,5R)-6-(3-chloro-6-(2-(2-cyclopropyl-1H-imidazol-1-yl)-4-fluorophenoxy)-1,2,4-triazin-5-yl)-2,6-diazabicyclo[3.2.1]octane-2-carboxylate (340 mg, 50%) as a brown oil. m/z: ES⁺ [M+H]⁺=542.25.

### tert-butyl (1S,5R)-6-(3-chloro-6-(4-fluoro-2-(2-isopropyl-1H-imidazol-1-yl) phenoxy)-1,2,4-triazin-5-yl)-2,6-diazabicyclo[3.2.1]octane-2-carboxylate

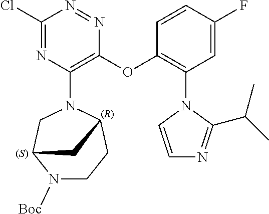

tert-Butyl (1S,5R)-6-(3,6-dichloro-1,2,4-triazin-5-yl)-2,6-diazabicyclo[3.2.1]octane-2-carboxylate (300 mg, 0.8 mmol, 1.0 equiv) and 4-fluoro-2-(2-isopropyl-1H-imidazol-1-yl)phenol (220 mg, 1.0 mmol, 1.2 equiv) were coupled according to general procedure C₁. The residue was purified using method RFPC4 (10% to 50% gradient in 10 min) to afford tert-butyl (1S,5R)-6-(3-chloro-6-(4-fluoro-2-(2-isopropyl-1H-imidazol-1-yl)phenoxy)-1,2,4-triazin-5-yl)-2,6-diazabicyclo[3.2.1]octane-2-carboxylate (300 mg, 66%) as a yellow oil. m/z: ES⁺ [M+H]⁺=544.03.

### tert-butyl (1S,5R)-6-(3-chloro-6-(2-(2-cyclopropyl-5-methyl-1H-imidazol-1-yl)-4-fluorophenoxy)-1,2,4-triazin-5-yl)-2,6-diazabicyclo[3.2.1]octane-2-carboxylate

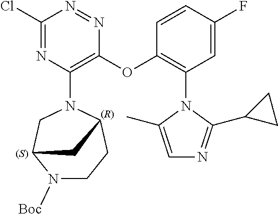

tert-Butyl (1S,5R)-6-(3,6-dichloro-1,2,4-triazin-5-yl)-2,6-diazabicyclo[3.2.1]octane-2-carboxylate (100 mg, 0.3 mmol, 1.0 equiv) and 2-(2-cyclopropyl-5-methylimidazol-1-yl)-4-fluorophenol (77 mg, 0.3 mmol, 1.2 equiv) were coupled according to general procedure C₁. The residue was purified using method RFPC4 (10% to 50% gradient in 10 min) to afford tert-butyl (1S,5R)-6-(3-chloro-6-(2-(2-cyclopropyl-5-methyl-1H-imidazol-1-yl)-4-fluorophenoxy)-1,2,4-triazin-5-yl)-2,6-diazabicyclo[3.2.1]octane-2-carboxylate (100 mg, 65%) as a yellow solid. m/z: ES⁺ [M+H]⁺=556.05.

### tert-butyl (1S,5R)-6-(3-chloro-6-(3-chloro-6-(4-fluoro-2-(1-isopropyl-1H-imidazol-5-yl)phenoxy)-1,2,4-triazin-5-yl)-2,6-diazabicyclo[3.2.1]octane-2-carboxylate

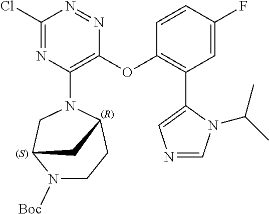

tert-Butyl (1S,5R)-6-(3,6-dichloro-1,2,4-triazin-5-yl)-2,6-diazabicyclo[3.2.1]octane-2-carboxylate (450 mg, 1.3 mmol) and 4-fluoro-2-(1-isopropyl-1H-imidazol-5-yl)phenol (413 mg, 1.9 mmol) were coupled according to general procedure C₁. The residue was purified using method RFPC1 (30% to 70% gradient in 20 min) to afford the title compound (360 mg, 53%) as an off-white solid. m/z: ES⁺ [M+H]⁺=544.20.

### tert-butyl (1S,5R)-6-(3-chloro-6-(2-(4-cyclopropylpyrimidin-5-yl)-4-fluorophenoxy)-1,2,4-triazin-5-yl)-2,6-diazabicyclo[3.2.1]octane-2-carboxylate

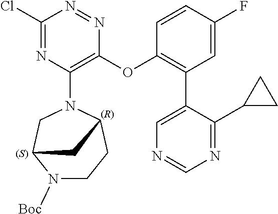

tert-Butyl (1S,5R)-6-(3,6-dichloro-1,2,4-triazin-5-yl)-2,6-diazabicyclo[3.2.1]octane-2-carboxylate (500 mg, 1.4 mmol, 1.0 equiv) and 2-(4-cyclopropylpyrimidin-5-yl)-4-fluorophenol (320 mg, 1.4 mmol, 1.0 equiv) were coupled according to general procedure C₁. The resulting residue was purified using method RFPC3 (10% to 100% gradient in 15 min) to afford tert-butyl (1S,5R)-6-(3-chloro-6-(2-(4-cyclopropylpyrimidin-5-yl)-4-fluorophenoxy)-1,2,4-triazin-5-yl)-2,6-diazabicyclo[3.2.1]octane-2-carboxylate (600 mg, 78%) as a yellow solid. m/z: ES⁺ [M+H]⁺=554.20.

### tert-butyl (1S,5R)-6-(3-chloro-6-(2-(4-cyclopropylpyridin-3-yl)-4-fluorophenoxy)-1,2,4-triazin-5-yl)-2,6-diazabicyclo[3.2.1]octane-2-carboxylate

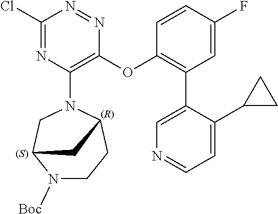

tert-Butyl (1S,5R)-6-(3,6-dichloro-1,2,4-triazin-5-yl)-2,6-diazabicyclo[3.2.1]octane-2-carboxylate (400 mg, 1.1 mmol, 1.0 equiv) and 2-(4-cyclopropylpyridin-3-yl)-4-fluorophenol (305 mg, 1.3 mmol, 1.2 equiv) were coupled according to general procedure C₁. The resulting residue was purified using method RFPC1 (40% to 80% gradient in 10 min) to afford tert-butyl (1S,5R)-6-(3-chloro-6-(2-(4-cyclopropylpyridin-3-yl)-4-fluorophenoxy)-1,2,4-triazin-5-yl)-2,6-diazabicyclo[3.2.1]octane-2-carboxylate (400 mg, 65%) as a light yellow solid. m/z: ES⁺ [M+H]⁺=553.20.

### tert-butyl (1S,5R)-6-(3-chloro-6-(2-(2-cyclopropylpyridin-3-yl)-4-fluorophenoxy)-1,2,4-triazin-5-yl)-2,6-diazabicyclo[3.2.1]octane-2-carboxylate

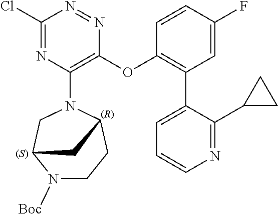

tert-Butyl (1S,5R)-6-(3,6-dichloro-1,2,4-triazin-5-yl)-2,6-diazabicyclo[3.2.1]octane-2-carboxylate (236 mg, 0.654 mmol, 1 equiv) and 2-(2-cyclopropylpyridin-3-yl)-4-fluorophenol (150 mg, 0.654 mmol, 1 equiv) were coupled according to general procedure C₁. The resulting residue was purified using method RPFC4 (10% to 50% gradient in 10 min) to afford tert-butyl (1S,5R)-6-(3-chloro-6-(2-(4-cyclopropylpyridin-3-yl)-4-fluorophenoxy)-1,2,4-triazin-5-yl)-2,6-diazabicyclo[3.2.1]octane-2-carboxylate (100 mg, 28% as a colourless solid. m/z: ES⁺ [M+H]⁺=533.04.

### tert-butyl (1S,5R)-6-(3-chloro-6-(2-(5-cyclopropylpyrimidin-4-yl)-4-fluorophenoxy)-1,2,4-triazin-5-yl)-2,6-diazabicyclo[3.2.1]octane-2-carboxylate

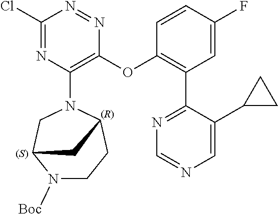

tert-Butyl (1S,5R)-6-(3,6-dichloro-1,2,4-triazin-5-yl)-2,6-diazabicyclo[3.2.1]octane-2-carboxylate (235 mg, 0.651 mmol, 1.00 equiv) and 2-(5-cyclopropylpyrimidin-4-yl)-4-fluorophenol (150 mg, 0.651 mmol, 1.00 equiv) were coupled according to general procedure C₁. The resulting residue was purified using method RPFC1 (20% to 100% gradient in 25 min) to afford tert-butyl (1S,5R)-6-(3-chloro-6-(2-(5-cyclopropylpyrimidin-4-yl)-4-fluorophenoxy)-1,2,4-triazin-5-yl)-2,6-diazabicyclo[3.2.1]octane-2-carboxylate (320 mg, 89%) as a yellow solid. m/z: ES⁺ [M+H]⁺=554.20.

### tert-butyl (1S,5R)-6-(3-chloro-6-(2-(3-cyclopropylpyrazin-2-yl)-4-fluorophenoxy)-1,2,4-triazin-5-yl)-2,6-diazabicyclo[3.2.1]octane-2-carboxylate

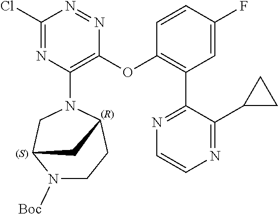

tert-Butyl (1S,5R)-6-(3,6-dichloro-1,2,4-triazin-5-yl)-2,6-diazabicyclo[3.2.1]octane-2-carboxylate (670 mg, 1.86 mmol, 1.02 equiv) and 2-(3-cyclopropylpyrazin-2-yl)-4-fluorophenol (420 mg, 1.82 mmol, 1.00 equiv) were coupled according to general procedure C₁. The resulting residue was purified using method RPFC3 (0% to 100% gradient in 30 min) to afford tert-butyl (1S,5R)-6-(3-chloro-6-(2-(3-cyclopropylpyrazin-2-yl)-4-fluorophenoxy)-1,2,4-triazin-5-yl)-2,6-diazabicyclo[3.2.1]octane-2-carboxylate (380 mg, 38%) as a colourless solid. m/z: ES⁺ [M+H]⁺=554.20.

### tert-butyl (1S,5R)-6-(6-(2-(2-(tert-butyl)-5-methyl-1H-imidazol-1-yl)-4-fluorophenoxy)-3-chloro-1,2,4-triazin-5-yl)-2,6-diazabicyclo[3.2.1]octane-2-carboxylate

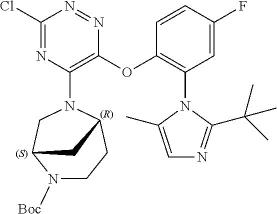

tert-Butyl (1S,5R)-6-(3,6-dichloro-1,2,4-triazin-5-yl)-2,6-diazabicyclo[3.2.1]octane-2-carboxylate (160 mg, 0.44 mmol, 1.1 equiv) and 2-(2-tert-butyl-5-methylimidazol-1-yl)-4-fluorophenol (100 mg, 0.40 mmol, 1.0 equiv) were coupled according to general procedure C₁. The residue was purified using method RFPC1 (10% to 50% gradient in 10 min) to afford tert-butyl (1S,5R)-6-(6-(2-(2-(tert-butyl)-5-methyl-1H-imidazol-1-yl)-4-fluorophenoxy)-3-chloro-1,2,4-triazin-5-yl)-2,6-diazabicyclo[3.2.1]octane-2-carboxylate (210 mg, 91%) as a yellow solid. m/z: ES⁺ [M+H]⁺=572.30.

### tert-butyl (1S,5R)-6-(3-chloro-6-(2-(5-cyclopropyl-2-methyl-1H-imidazol-1-yl)-4-fluorophenoxy)-1,2,4-triazin-5-yl)-2,6-diazabicyclo[3.2.1]octane-2-carboxylate

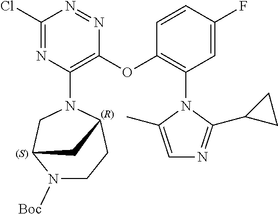

tert-Butyl (1S,5R)-6-(3,6-dichloro-1,2,4-triazin-5-yl)-2,6-diazabicyclo[3.2.1]octane-2-carboxylate (600 mg, 1.7 mmol, 1.0 equiv) and 2-(5-cyclopropyl-2-methyl-1H-imidazol-1-yl)-4-fluorophenol (387 mg, 1.7 mmol, 1.0 equiv) were coupled according to general procedure C₁. The resulting mixture was diluted with water (100 mL) and extracted with DCM (2×100 mL). The combined organic layers were washed with brine (2×200 mL), dried over Na₂SO₄ and concentrated under reduced pressure. The residue was purified using method RPFC1 (30% to 70% gradient in 10 min), to give tert-butyl (1S,5R)-6-(3-chloro-6-(2-(5-cyclopropyl-2-methyl-1H-imidazol-1-yl)-4-fluorophenoxy)-1,2,4-triazin-5-yl)-2,6-diazabicyclo[3.2.1]octane-2-carboxylate (340 mg, 37%) as a yellow oil. m/z: ES⁺ [M+H]⁺=556.25.

### tert-butyl (1S,5R)-6-(3-chloro-6-[2-(2-cyclopropyl-5-isopropylimidazol-1-yl)-4-fluorophenoxy]-1,2,4-triazin-5-yl)-2,6-diazabicyclo[3.2.1]octane-2-carboxylate

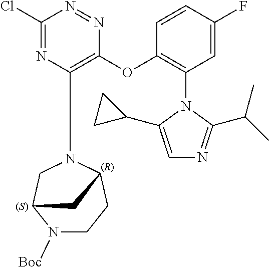

tert-Butyl (1S,5R)-6-(3,6-dichloro-1,2,4-triazin-5-yl)-2,6-diazabicyclo[3.2.1]octane-2-carboxylate (360 mg, 0.9 mmol, 1.0 equiv) and 2-(5-cyclopropyl-2-isopropylimidazol-1-yl)-4-fluorophenol (236 mg, 0.9 mmol, 1.0 equiv) were coupled according to general procedure C₁. The residue was purified using method RFPC1 (10% to 50% gradient in 10 min) to afford tert-butyl (1S,5R)-6-(3-chloro-6-[2-(2-cyclopropyl-5-isopropylimidazol-1-yl)-4-fluorophenoxy]-1,2,4-triazin-5-yl)-2,6-diazabicyclo[3.2.1]octane-2-carboxylate (520 mg, 98%) as a yellow solid. m/z: ES⁺ [M+H]⁺=584.35.

### tert-butyl (1S,5R)-6-(3-chloro-6-(4-fluoro-2-(isopropyl(methyl)carbamoyl)phenoxy)-1,2,4-triazin-5-yl)-2,6-diazabicyclo[3.2.1]octane-2-carboxylate

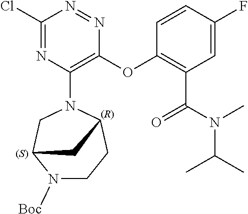

tert-Butyl (1S,5R)-6-(3,6-dichloro-1,2,4-triazin-5-yl)-2,6-diazabicyclo[3.2.1]octane-2-carboxylate (500 mg, 1.39 mmol, 1 equiv) and 5-fluoro-2-hydroxy-N-isopropyl-N-methylbenzamide methylbenzamide (352 mg, 1.7 mmol, 1.2 equiv) were coupled according to general procedure C₁. The residue was purified using method RFPC1 (40% to 80% gradient in 10 min) to afford tert-butyl (1S,5R)-6-(3-chloro-6-(4-fluoro-2-(isopropyl(methyl)carbamoyl)phenoxy)-1,2,4-triazin-5-yl)-2,6-diazabicyclo[3.2.1]octane-2-carboxylate (600 mg, 81%) as a colourless solid. m/z: ES⁺ [M+H]⁺=535.25.

### tert-butyl (1S,5R)-6-(3-chloro-6-(2-(2,5-dicyclopropyl-1H-imidazol-1-yl)-4-fluorophenoxy)-1,2,4-triazin-5-yl)-2,6-diazabicyclo[3.2.1]octane-2-carboxylate

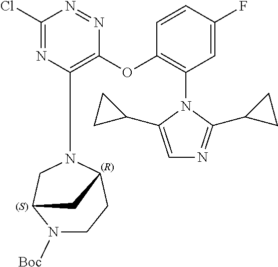

tert-Butyl (1S,5R)-6-(3,6-dichloro-1,2,4-triazin-5-yl)-2,6-diazabicyclo[3.2.1]octane-2-carboxylate (2.0 g, 5.5 mmol, 1 equiv) and 2-(2,5-dicyclopropyl-1H-imidazol-1-yl)-4-fluorophenol (2.9 g, 11 mmol, 2 equiv) were coupled according to general procedure C₁. The residue was purified using method RFPC1 (10% to 90% gradient in 20 min) to afford tert-butyl (1S,5R)-6-(3-chloro-6-(2-(2,5-dicyclopropyl-1H-imidazol-1-yl)-4-fluorophenoxy)-1,2,4-triazin-5-yl)-2,6-diazabicyclo[3.2.1]octane-2-carboxylate (2.9 g, 90%) as a yellow solid. m/z: ES⁺ [M+H]⁺=582.00.

### tert-butyl (1S,5R)-6-(3-chloro-6-(2-(N-ethylcyclopropanecarboxamido)-4-fluorophenoxy)-1,2,4-triazin-5-yl)-2,6-diazabicyclo[3.2.1]octane-2-carboxylate

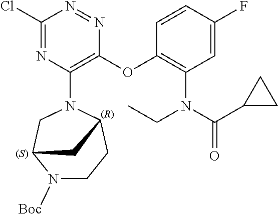

tert-Butyl (1S,5R)-6-(3,6-dichloro-1,2,4-triazin-5-yl)-2,6-diazabicyclo[3.2.1]octane-2-carboxylate (300 mg, 0.83 mmol, 1.0 equiv) and N-ethyl-N-(5-fluoro-2-hydroxyphenyl)cyclopropanecarboxamide (185 mg, 0.83 mmol, 1.0 equiv) were coupled according to general procedure C₁. The resulting mixture was diluted with water (10 mL) and extracted with EtOAc (3×10 mL). The combined organic layers were washed with brine (10 mL), dried over Na₂SO₄, and concentrated under reduced pressure. The residue was purified by silica gel column chromatography, eluting with DCM/MeOH (12:1) to afford tert-butyl (1S,5R)-6-(3-chloro-6-(2-(N-ethylcyclopropanecarboxamido)-4-fluorophenoxy)-1,2,4-triazin-5-yl)-2,6-diazabicyclo[3.2.1]octane-2-carboxylate (240 mg, 53%) as a light yellow oil. m/z: ES⁺ [M+H]⁺=547.35.

### tert-butyl (1S,5R)-6-(3-chloro-6-(4-fluoro-2-(N-isopropylcyclopropanecarboxamido)phenoxy)-1,2,4-triazin-5-yl)-2,6-diazabicyclo[3.2.1]octane-2-carboxylate

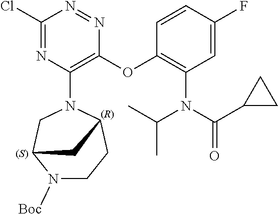

tert-Butyl (1S,5R)-6-(3,6-dichloro-1,2,4-triazin-5-yl)-2,6-diazabicyclo[3.2.1]octane-2-carboxylate (200 mg, 0.56 mmol, 1.0 equiv) and N-(5-fluoro-2-hydroxyphenyl)-N-isopropylcyclopropane carboxamide (132 mg, 0.56 mmol, 1.0 equiv) were coupled according to general procedure C₁. The resulting mixture was diluted with water (10 mL) and extracted with EtOAc (3×20 mL). The combined organic layers were washed with brine (10 mL), dried over Na₂SO₄, and concentrated under reduced pressure. The residue was purified by silica gel column chromatography (PE/EtOAc 1:1) to afford tert-butyl (1S,5R)-6-(3-chloro-6-(4-fluoro-2-(N-isopropylcyclopropanecarboxamido)phenoxy)-1,2,4-triazin-5-yl)-2,6-diazabicyclo[3.2.1]octane-2-carboxylate (300 mg, 96%) as a light yellow oil. m/z: ES⁺ [M+H]⁺=561.30.

### tert-butyl (1S,5R)-6-(3-chloro-6-(4-fluoro-2-(N-isopropylisobutyramido)phenoxy)-1,2,4-triazin-5-yl)-2,6-diazabicyclo[3.2.1]octane-2-carboxylate

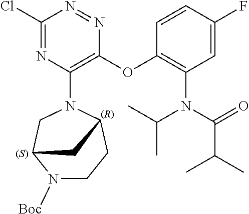

tert-Butyl (1S,5R)-6-(3,6-dichloro-1,2,4-triazin-5-yl)-2,6-diazabicyclo[3.2.1]octane-2-carboxylate (225 mg, 0.63 mmol, 1.0 equiv) and N-(5-fluoro-2-hydroxyphenyl)-N-isopropylisobutyramide (150 mg, 0.63 mmol, 1.0 equiv) were coupled according to general procedure C₁. The resulting mixture was diluted with water (20 mL) and extracted with DCM (3×20 mL). The combined organics were dried over Na₂SO₄ and filtered. The filtrate was concentrated under reduced pressure and the residue purified by RPFC1 (20% to 70% gradient in 20 min) to afford tert-butyl(1S,5R)-6-(3-chloro-6-(4-fluoro-2-(N-isopropylisobutyramido)phenoxy)-1,2,4-triazin-5-yl)-2,6-diazabicyclo[3.2.1]octane-2-carboxylate (330 mg, 93%) as a colourless solid. m/z: ES⁺ [M+H]⁺=563.30.

### tert-butyl (1S,5R)-6-(3-chloro-6-(2-(N-cyclopropylcyclopropanecarboxamido)-4-fluorophenoxy)-1,2,4-triazin-5-yl)-2,6-diazabicyclo[3.2.1]octane-2-carboxylate

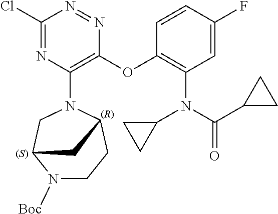

tert-Butyl (1S,5R)-6-(3,6-dichloro-1,2,4-triazin-5-yl)-2,6-diazabicyclo[3.2.1]octane-2-carboxylate (500 mg, 1.4 mmol, 1.0 equiv) andN-cyclopropyl-N-(5-fluoro-2-hydroxyphenyl)cyclopro panecarboxamide (653 mg, 2.8 mmol, 2.0 equiv) were coupled according to general procedure C₁. The resulting mixture was filtered, the filter cake was washed with THF (3×3 mL), then the filtrate was concentrated under reduced pressure. The residue was purified by Prep-TLC (PE/EtOAC; 2:1) to afford tert-butyl (1S,5R)-6-(3-chloro-6-(2-(N-cyclopropylcyclopropanecarboxamido)-4-fluorophenoxy)-1,2,4-triazin-5-yl)-2,6-diazabicyclo[3.2.1]octane-2-carboxylate (500 mg, 64%) as a yellow solid. m/z: ES⁺ [M+H]⁺=559.20.

### tert-butyl (1S,5R)-6-(3-chloro-6-(2-(4-cyclopropyl-6-methylpyrimidin-5-yl)-4-fluorophenoxy)-1,2,4-triazin-5-yl)-2,6-diazabicyclo[3.2.1]octane-2-carboxylate

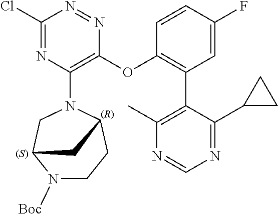

tert-Butyl (1S,5R)-6-(3,6-dichloro-1,2,4-triazin-5-yl)-2,6-diazabicyclo[3.2.1]octane-2-carboxylate (1.1 g, 3.0 mmol, 1.2 equiv) and 2-(4-cyclopropyl-6-methylpyrimidin-5-yl)-4-fluorophenol (600 mg, 2.5 mmol, 1.0 equiv) were coupled according to general procedure C₁. The resulting mixture was diluted with water (50 mL) and extracted with DCM (3×50 mL), dried over Na₂SO₄, and concentrated under reduced pressure. The residue was purified using method RPFC1 (10% to 100% gradient in 20 min) to afford tert-butyl (1S,5R)-6-(3-chloro-6-(2-(4-cyclopropyl-6-methylpyrimidin-5-yl)-4-fluorophenoxy)-1,2,4-triazin-5-yl)-2,6-diazabicyclo[3.2.1]octane-2-carboxylate (900 mg, 65%) as a yellow solid. m/z: ES⁺ [M+H]⁺=568.22.

### tert-butyl (1S,5R)-6-(3-chloro-6-(4-fluoro-2-(2-isopropyl-5-methyl-1H-imidazol-1-yl)phenoxy)-1,2,4-triazin-5-yl)-2,6-diazabicyclo[3.2.1]octane-2-carboxylate

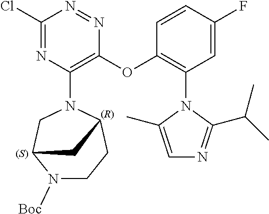

tert-Butyl (1S,5R)-6-(3,6-dichloro-1,2,4-triazin-5-yl)-2,6-diazabicyclo[3.2.1]octane-2-carboxylate (500 mg, 1.4 mmol, 1.0 equiv) and 4-fluoro-2-(2-isopropyl-5-methyl-1H-imidazol-1-yl)phenol (390 mg, 1.7 mmol, 1.2 equiv) were coupled according to general procedure C₁. The residue was purified using method RFPC1 (10% to 100% gradient in 20 min) to afford tert-butyl (1S,5R)-6-(3-chloro-6-(4-fluoro-2-(2-isopropyl-5-methyl-1H-imidazol-1-yl)phenoxy)-1,2,4-triazin-5-yl)-4-fluorophenoxy)-1,2,4-triazin-5-yl)-2,6-diazabicyclo[3.2.1]octane-2-carboxylate (600 mg, 77%) as a light yellow solid. m/z: ES⁺ [M+H]⁺=558.35.

### tert-butyl (1S,5R)-6-(3-chloro-6-(4-fluoro-2-(2-isopropyl-4,5-dimethyl-1H-imidazol-1-yl)phenoxy)-1,2,4-triazin-5-yl)-2,6-diazabicyclo[3.2.1]octane-2-carboxylate

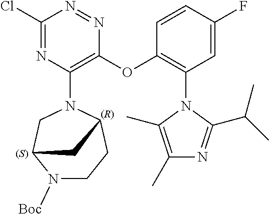

tert-Butyl (1S,5R)-6-(3,6-dichloro-1,2,4-triazin-5-yl)-2,6-diazabicyclo[3.2.1]octane-2-carboxylate (6.5 g, 18 mmol, 1.0 equiv) and 4-fluoro-2-(2-isopropyl-4,5-dimethyl-1H-imidazol-1-yl)phenol (4.5 g, 18 mmol, 1.0 equiv) were coupled according to general procedure C₁. The resulting mixture was diluted with water (100 mL), extracted with DCM (3×100 mL), dried over Na₂SO₄, and concentrated under reduced pressure. The residue was purified using method RPFC1 (40% to 70% gradient in 20 min) to afford tert-butyl (1S,5R)-6-(3-chloro-6-(4-fluoro-2-(2-isopropyl-4,5-dimethyl-1H-imidazol-1-yl)phenoxy)-1,2,4-triazin-5-yl)-2,6-diazabicyclo[3.2.1]octane-2-carboxylate (5.0 g, 48%) as a yellow solid. m/z: ES⁺ [M+H]⁺=572.25.

### tert-butyl (1S,5R)-6-(3-chloro-6-(2-(5-cyclopropyl-2,4-dimethyl-1H-imidazol-1-yl)-4-fluorophenoxy)-1,2,4-triazin-5-yl)-2,6-diazabicyclo[3.2.1]octane-2-carboxylate

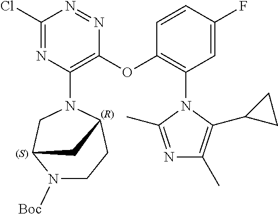

tert-Butyl (1S,5R)-6-(3,6-dichloro-1,2,4-triazin-5-yl)-2,6-diazabicyclo[3.2.1]octane-2-carboxylate (100 mg, 0.28 mmol, 1.0 equiv) and 2-(5-cyclopropyl-2,4-dimethyl-1H-imidazol-1-yl)-4-fluorophenol (68 mg, 0.28 mmol, 1.0 equiv) were coupled according to general procedure C₁. The residue was purified using method RFPC1 (10% to 100% gradient in 20 min) to afford tert-butyl (1S,5R)-6-(3-chloro-6-(2-(5-cyclopropyl-2,4-dimethyl-1H-imidazol-1-yl)-4-fluorophenoxy)-1,2,4-triazin-5-yl)-2,6-diazabicyclo[3.2.1]octane-2-carboxylate (130 mg, 82%) as a yellow solid. m/z: ES⁺ [M+H]⁺=570.20.

### tert-butyl (1S,5R)-6-(3-chloro-6-(4-fluoro-2-(2-methoxy-5-methyl-1H-imidazol-1-yl)phenoxy)-1,2,4-triazin-5-yl)-2,6-diazabicyclo[3.2.1]octane-2-carboxylate

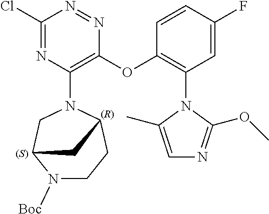

tert-Butyl (1S,5R)-6-(3,6-dichloro-1,2,4-triazin-5-yl)-2,6-diazabicyclo[3.2.1]octane-2-carboxylate (130 mg, 0.36 mmol, 1.0 equiv) and 4-fluoro-2-(2-methoxy-5-methyl-1H-imidazol-1-yl)phenol (80 mg, 0.36 mmol, 1.0 equiv) were coupled according to general procedure C₁. The residue was purified using method RFPC1 (10% to 100% gradient in 25 min) to afford tert-butyl (1S,5R)-6-(3-chloro-6-(4-fluoro-2-(2-methoxy-5-methyl-1H-imidazol-1-yl)phenoxy)-1,2,4-triazin-5-yl)-2,6-diazabicyclo[3.2.1]octane-2-carboxylate (180 mg, 91%) as a brown solid. m/z: ES⁺ [M+H]⁺=546.10.

### tert-butyl (1S,5R)-6-(3-chloro-6-(4-fluoro-2-(2-isopropoxy-5-methyl-1H-imidazol-1-yl)phenoxy)-1,2,4-triazin-5-yl)-2,6-diazabicyclo[3.2.1]octane-2-carboxylate

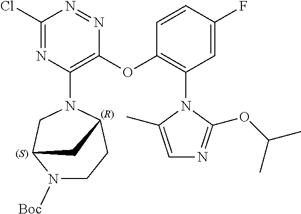

tert-Butyl (1S,5R)-6-(3,6-dichloro-1,2,4-triazin-5-yl)-2,6-diazabicyclo[3.2.1]octane-2-carboxylate (158 mg, 0.44 mmol, 1.0 equiv) and 4-fluoro-2-(2-isopropoxy-5-methyl-1H-imidazol-1-yl)phenol (110 mg, 0.44 mmol, 1.0 equiv) were coupled according to general procedure C₁. The residue was purified using method RFPC1 (30% to 90% gradient in 20 min) to afford tert-butyl (1S,5R)-6-(3-chloro-6-(4-fluoro-2-(2-isopropoxy-5-methyl-1H-imidazol-1-yl) phenoxy)-1,2,4-triazin-5-yl)-2,6-diazabicyclo[3.2.1]octane-2-carboxylate (220 mg, 87.20%) as a yellow solid. m/z: ES⁺ [M+H]⁺=574.25.

### tert-butyl (1S,5R)-6-(3-chloro-6-(2-(3-cyclopropyl-5-methyl-4H-1,2,4-triazol-4-yl)-4-fluorophenoxy)-1,2,4-triazin-5-yl)-2,6-diazabicyclo[3.2.1]octane-2-carboxylate

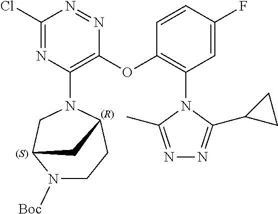

tert-Butyl (1S,5R)-6-(3,6-dichloro-1,2,4-triazin-5-yl)-2,6-diazabicyclo[3.2.1]octane-2-carboxylate (300 mg, 0.83 mmol, 1.0 equiv) and 2-(3-cyclopropyl-5-methyl-4H-1,2,4-triazol-4-yl)-4-fluorophenol (194 mg, 0.83 mmol, 1.0 equiv) were coupled according to general procedure C₁. The residue was purified using method RFPC1 (10% to 100% gradient in 20 min) to afford tert-butyl (1S,5R)-6-(3-chloro-6-(2-(3-cyclopropyl-5-methyl-4H-1,2,4-triazol-4-yl)-4-fluorophenoxy)-1,2,4-triazin-5-yl)-2,6-diazabicyclo[3.2.1]octane-2-carboxylate (450 mg, 97%) as a yellow solid. m/z: ES⁺ [M+H]⁺=557.25.

### tert-butyl (1S,5R)-6-(3-chloro-6-(2-(3,5-dicyclopropyl-4H-1,2,4-triazol-4-yl)-4-fluorophenoxy)-1,2,4-triazin-5-yl)-2,6-diazabicyclo[3.2.1]octane-2-carboxylate

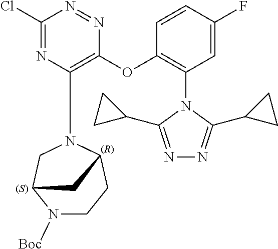

tert-Butyl (1S,5R)-6-(3,6-dichloro-1,2,4-triazin-5-yl)-2,6-diazabicyclo[3.2.1]octane-2-carboxylate (300 mg, 0.83 mmol, 1.0 equiv), and 2-(3,5-dicyclopropyl-4H-1,2,4-triazol-4-yl)-4-fluorophenol (216 mg, 0.83 mmol, 1.0 equiv) were coupled according to general procedure C₁. The resulting mixture was concentrated under reduced pressure. The residue was purified using method RPFC1 (10% to 100% gradient in 20 min) to afford tert-butyl (1S,5R)-6-(3-chloro-6-(2-(3,5-dicyclopropyl-4H-1,2,4-triazol-4-yl)-4-fluorophenoxy)-1,2,4-triazin-5-yl)-2,6-diazabicyclo[3.2.1]octane-2-carboxylate (320 mg, 66%) as a yellow solid. m/z: ES⁺ [M+H]⁺=583.10.

### tert-butyl (1S,5R)-6-(3-chloro-6-(2-(3,5-diisopropyl-4H-1,2,4-triazol-4-yl)-4-fluorophenoxy)-1,2,4-triazin-5-yl)-2,6-diazabicyclo[3.2.1]octane-2-carboxylate

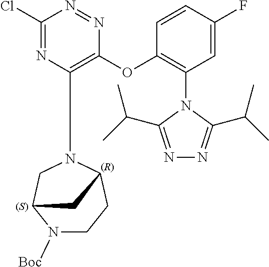

tert-Butyl (1S,5R)-6-(3,6-dichloro-1,2,4-triazin-5-yl)-2,6-diazabicyclo[3.2.1]octane-2-carboxylate (400 mg, 1.1 mmol, 1.0 equiv) and 2-(3,5-diisopropyl-4H-1,2,4-triazol-4-yl)-4-fluorophenol (292 mg, 1.1 mmol, 1.0 equiv) were coupled according to general procedure C₁. The residue was purified using method RPFC1 (10% to 90% gradient in 20 min) to afford tert-butyl (1S,5R)-6-(3-chloro-6-(2-(3,5-diisopropyl-4H-1,2,4-triazol-4-yl)-4-fluorophenoxy)-1,2,4-triazin-5-yl)-2,6-diazabicyclo[3.2.1]octane-2-carboxylate (600 mg, 92%) as a yellow solid. m/z: ES⁺ [M+H]⁺=587.20.

### tert-butyl (1S,5R)-6-(3-chloro-6-(4-fluoro-2-(4-cyclopropyl-1-methyl-1H-pyrazol-5-yl)phenoxy)-1,2,4-triazin-5-yl)-2,6-diazabicyclo[3.2.1]octane-2-carboxylate

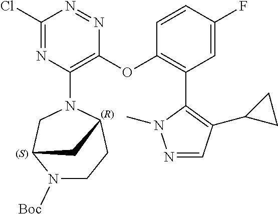

tert-Butyl (1S,5R)-6-(3,6-dichloro-1,2,4-triazin-5-yl)-2,6-diazabicyclo[3.2.1]octane-2-carboxylate (200 mg, 0.555 mmol, 1 equiv), 2-(4-cyclopropyl-1-methyl-1H-pyrazol-5-yl)-4-fluorophenol (155 mg, 0.67 mmol, 1.2 equiv) were coupled according to general procedure C₁. The resulting mixture was diluted with water (50 mL) and extracted with EtOAc (3×200 mL), then dried over Na₂SO₄. After filtration, the filtrate was concentrated under reduced pressure, and was purified using method RPFC1 (10% to 100% gradient in 20 min) to afford tert-butyl (1S,5R)-6-(3-chloro-6-(2-(4-cyclopropyl-1-methyl-1H-pyrazol-5-yl)-4-fluorophenoxy)-1,2,4-triazin-5-yl)-2,6-diazabicyclo[3.2.1]octane-2-carboxylate (250 mg, 81%) as a light brown solid. m/z: ES⁺ [M+H]⁺=556.25.

### tert-butyl (1S,5R)-6-(3-chloro-6-(4-fluoro-2-(4-isopropyl-1-methyl-1H-pyrazol-5-yl)phenoxy)-1,2,4-triazin-5-yl)-2,6-diazabicyclo[3.2.1]octane-2-carboxylate

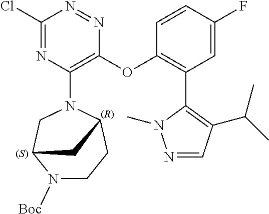

tert-Butyl (1S,5R)-6-(3,6-dichloro-1,2,4-triazin-5-yl)-2,6-diazabicyclo[3.2.1]octane-2-carboxylate (200 mg, 0.555 mmol, 1 equiv) and 4-fluoro-2-(4-isopropyl-1-methyl-1H-pyrazol-5-yl)phenol (130 mg, 0.555 mmol, 1 equiv) were coupled according to general procedure C₁. The resulting mixture was diluted with water (30 mL) and extracted with EtOAc (3×30 mL), dried over anhydrous Na₂SO₄. After filtration, the filtrate was concentrated under reduced pressure. The residue product was purified using method RPFC1 (10% to 100% gradient in 20 min) to afford tert-butyl (1S,5R)-6-(3-chloro-6-(4-fluoro-2-(4-isopropyl-1-methyl-1H-pyrazol-5-yl)phenoxy)-1,2,4-triazin-5-yl)-2,6-diazabicyclo[3.2.1]octane-2-carboxylate (250 mg, 81%) as a light brown solid. m/z: ES⁺ [M+H]⁺=558.25.

### tert-butyl (1S,5R)-6-(3-chloro-6-(2-(4-cyclopropyl-1-isopropyl-1H-pyrazol-5-yl)-4-fluorophenoxy)-1,2,4-triazin-5-yl)-2,6-diazabicyclo[3.2.1]octane-2-carboxylate

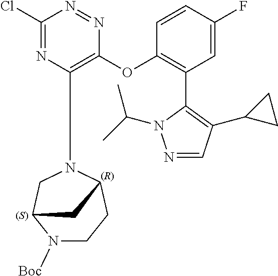

tert-Butyl (1S,5R)-6-(3,6-dichloro-1,2,4-triazin-5-yl)-2,6-diazabicyclo[3.2.1]octane-2-carboxylate (300 mg, 0.83 mmol, 1.0 equiv) and 2-(4-cyclopropyl-1-isopropyl-1H-pyrazol-5-yl)-4-fluorophenol (220 mg, 0.83 mmol, 1 equiv) were coupled according to general procedure C₁. The resulting mixture was diluted with water (40 mL), and extracted with EtOAc (2×50 mL). The combined organic layers were washed with brine (100 mL), dried over Na₂SO₄ and filtered. The filtrate was concentrated under reduced pressure and purified by RPFC1 (20% to 80% gradient in 25 min) to afford tert-butyl (1S,5R)-6-(3-chloro-6-(2-(4-cyclopropyl-1-isopropyl-1H-pyrazol-5-yl)-4-fluorophenoxy)-1,2,4-triazin-5-yl)-2,6-diazabicyclo[3.2.1]octane-2-carboxylate (300 mg, 62%) as a light yellow solid. m/z: ES⁺ [M+H]⁺=584.10.

### tert-butyl (1S,5R)-6-(3-chloro-6-(4-fluoro-2-(1-methyl-6-oxo-1,6-dihydropyridin-2-yl)phenoxy)-1,2,4-triazin-5-yl)-2,6-diazabicyclo[3.2.1]octane-2-carboxylate

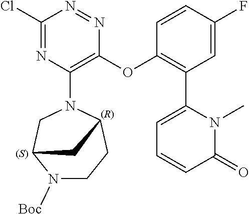

tert-Butyl (1S,5R)-6-(3,6-dichloro-1,2,4-triazin-5-yl)-2,6-diazabicyclo[3.2.1]octane-2-carboxylate and (592 mg, 1.64 mmol, 1.5 equiv) and 6-(5-fluoro-2-hydroxyphenyl)-1-methylpyridin-2-one (240 mg, 1.10 mmol, 1 equiv) were coupled according to general procedure C₁. The resulting residue was purified using method RPFC1 (40% to 80% gradient in 10 min) to afford tert-butyl (1S,5R)-6-(3-chloro-6-(4-fluoro-2-(1-methyl-6-oxo-1,6-dihydropyridin-2-yl)phenoxy)-1,2,4-triazin-5-yl)-2,6-diazabicyclo[3.2.1]octane-2-carboxylate (270 mg, 45%) as a colourless solid. m/z: ES⁺ [M+H]⁺=543.20.

### tert-butyl (1S,5R)-6-(5-(4-fluoro-2-(methoxycarbonyl)phenoxy)pyrimidin-4-yl)-2,6-diazabicyclo[3.2.1]octane-2-carboxylate

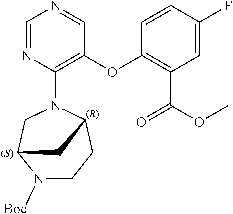

The coupling between tert-butyl (1S,5R)-2,6-diazabicyclo[3.2.1]octane-2-carboxylate (350 mg, 1.6 mmol, 0.9 equiv) and methyl 2-((4-chloropyrimidin-5-yl)oxy)-5-fluorobenzoate (500 mg, 1.8 mmol, 1.0 equiv) was performed according to general procedure G1. The resulting mixture was purified using method RFPC1 (0% to 100% gradient in 10 min), affording tert-butyl (1S,5R)-6-(5-(4-fluoro-2-(methoxycarbonyl)phenoxy)pyrimidin-4-yl)-2,6-diazabicyclo[3.2.1]octane-2-carboxylate (700 mg, 86%) as a yellow solid. m/z: ES⁺ [M+H]⁺=459.35.

### methyl 2-((4-((1S,5R)-2,6-diazabicyclo[3.2.1]octan-6-yl)pyrimidin-5-yl)oxy)-5-fluorobenzoate

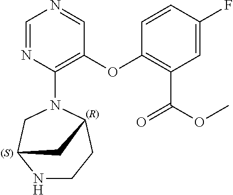

The Boc deprotection of tert-butyl (1S,5R)-6-(5-(4-fluoro-2-(methoxycarbonyl)phenoxy)pyrimidin-4-yl)-2,6-diazabicyclo[3.2.1]octane-2-carboxylate (250 mg, 0.5 mmol) was performed according to general procedure B1, affording crude methyl 2-((4-((1S,5R)-2,6-diazabicyclo[3.2.1]octan-6-yl)pyrimidin-5-yl)oxy)-5-fluorobenzoate as a yellow solid (300 mg, 96%), which used in next step without further purification. m/z: ES⁺ [M+H]⁺=359.25.

### tert-butyl (1S,5R)-6-(5-(4-fluoro-2-(methoxycarbonyl)phenoxy)pyrimidin-4-yl)-2,6-diazabicyclo[3.2.1]octane-2-carboxylate

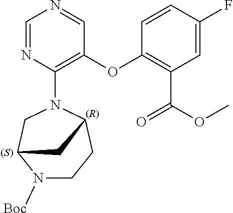

The coupling between tert-butyl (1S,5R)-2,6-diazabicyclo[3.2.1]octane-2-carboxylate (350 mg, 1.6 mmol, 0.9 equiv) and methyl 2-((4-chloropyrimidin-5-yl)oxy)-5-fluorobenzoate (500 mg, 1.8 mmol, 1.0 equiv) was performed according to general procedure G1. The resulting mixture was purified using method RFPC1 (0% to 100% gradient in 10 min), affording tert-butyl (1S,5R)-6-(5-(4-fluoro-2-(methoxycarbonyl)phenoxy)pyrimidin-4-yl)-2,6-diazabicyclo[3.2.1]octane-2-carboxylate (700 mg, 86%) as a yellow solid. m/z: ES⁺ [M+H]⁺=459.35.

### methyl 2-((4-((1S,5R)-2,6-diazabicyclo[3.2.1]octan-6-yl)pyrimidin-5-yl)oxy)-5-fluorobenzoate

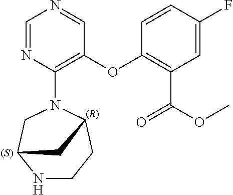

The Boc deprotection of tert-butyl (1S,5R)-6-(5-(4-fluoro-2-(methoxycarbonyl)phenoxy)pyrimidin-4-yl)-2,6-diazabicyclo[3.2.1]octane-2-carboxylate (250 mg, 0.5 mmol) was performed according to general procedure B1, affording crude methyl 2-((4-((1S,5R)-2,6-diazabicyclo[3.2.1]octan-6-yl)pyrimidin-5-yl)oxy)-5-fluorobenzoate as a yellow solid (300 mg, 96%), which used in next step without further purification. m/z: ES⁺ [M+H]⁺=359.25.

### tert-butyl (1S,5R)-6-(5-(2-bromo-4-fluorophenoxy)pyrimidin-4-yl)-2,6-diazabicyclo[3.2.1]octane-2-carboxylate

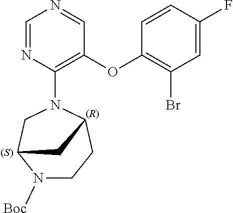

The coupling between tert-butyl (1S,5R)-2,6-diazabicyclo[3.2.1]octane-2-carboxylate (83 mg, 0.4 mmol, 1.0 equiv) and 5-(2-bromo-4-fluorophenoxy)-4-chloropyrimidine (125 mg, 0.4 mmol, 1.0 equiv) was performed according to general procedure G1. The resulting mixture was purified using method RFPC1 (0% to 100% gradient in 10 min), affording tert-butyl (1S,5R)-6-(5-(2-bromo-4-fluorophenoxy)pyrimidin-4-yl)-2,6-diazabicyclo[3.2.1]octane-2-carboxylate (190 mg, 96%) as a yellow solid. m/z: ES⁺ [M+H]⁺=481.35.

### 2-((4-((1S,5R)-2,6-diazabicyclo[3.2.1]octan-6-yl)pyrimidin-5-yl)oxy)-5-fluoro-N-isopropyl-N-methylbenzamide

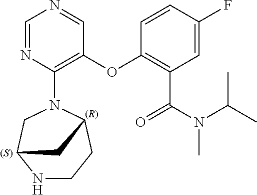

### Step 1: tert-butyl (1S,5R)-6-(5-(4-fluoro-2-(isopropyl(methyl) carbamoyl)phenoxy)pyrimidin-4-yl)-2,6-diazabicyclo[3.2.1]octane-2-carboxylate

The coupling between tert-butyl (1S,5R)-2,6-diazabicyclo[3.2.1]octane-2-carboxylate (163 mg, 0.8 mmol, 1.0 equiv) and 2-((4-chloropyrimidin-5-yl)oxy)-5-fluoro-N-isopropyl-N-methylbenzamide (250 mg, 0.8 mmol, 1.0 equiv) was performed according to general procedure G1. The resulting mixture was then diluted with water (10 mL), and extracted with DCM (3×10 mL). The combined organic layers were washed with brine (10 mL), and dried over Na₂SO₄. After filtration, the filtrate was concentrated under reduced pressure, and the residue was purified by silica gel column chromatography, eluted with PE/EtOAc (1:1) to afford tert-butyl (1S,5R)-6-(5-(4-fluoro-2-(isopropyl(methyl) carbamoyl)phenoxy)pyrimidin-4-yl)-2,6-diazabicyclo[3.2.1]octane-2-carboxylate (350 mg, 91%) as alight yellow oil. m/z: ES⁺ [M+H]⁺=500.30.

### Step 2: 2-((4-((1S,5R)-2,6-diazabicyclo[3.2.1]octan-6-yl)pyrimidin-5-yl)oxy)-5-fluoro-N-isopropyl-N-methylbenzamide

The Boc deprotection of tert-butyl (1S,5R)-6-(5-(4-fluoro-2-(isopropyl(methyl)carbamoyl)phenoxy)pyrimidin-4-yl)-2,6-diazabicyclo[3.2.1]octane-2-carboxylate (250 mg, 0.5 mmol) was performed according to general procedure B2, affording crude 2-((4-((1S,5R)-2,6-diazabicyclo[3.2.1]octan-6-yl)pyrimidin-5-yl)oxy)-5-fluoro-N-isopropyl-N-methylbenzamide (200 mg, quant.) as a light yellow oil. m/z: ES⁺ [M+H]⁺=400.15.

### 2-((4-((1S,5R)-2,6-diazabicyclo[3.2.1]octan-6-yl)pyrimidin-5-yl)oxy)-N-ethyl-5-fluoro-N-isopropylbenzamide

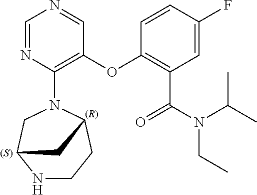

### Step 1: tert-Butyl (1S,5R)-6-(5-(2-(ethyl(isopropyl)carbamoyl)-4-fluorophenoxy)pyrimidin-4-yl)-2,6-diazabicyclo[3.2.1]octane-2-carboxylate

The coupling between tert-butyl (1S,5R)-2,6-diazabicyclo[3.2.1]octane-2-carboxylate (163 mg, 0.8 mmol, 1.2 equiv) and 2-((4-chloropyrimidin-5-yl)oxy)-N-ethyl-5-fluoro-N-isopropylbenzamide (3.0 g, 8.9 mmol, 1 equiv) was performed according to general procedure G1. The resulting mixture was diluted with water (30 mL) and extracted with EtOAc (2×100 mL). The combined organic layers were washed with brine (2×20 mL), dried over Na₂SO₄, and concentrated under reduced pressure. The residue was purified using method RFPC1 (10% to 80% gradient in 20 min), affording tert-butyl (1S,5R)-6-(5-(2-(ethyl(isopropyl)carbamoyl)-4-fluorophenoxy)pyrimidin-4-yl)-2,6-diazabicyclo[3.2.1]octane-2-carboxylate (3.2 g, 70%) as a yellow solid. m/z: ES⁺ [M+H]⁺=514.30.

### Step 2: 2-((4-((1S,5R)-2,6-diazabicyclo[3.2.1]octan-6-yl)pyrimidin-5-yl)oxy)-N-ethyl-5-fluoro-N-isopropylbenzamide

The Boc deprotection of tert-butyl (1S,5R)-6-(5-(2-(ethyl(isopropyl)carbamoyl)-4-fluorophenoxy)pyrimidin-4-yl)-2,6-diazabicyclo[3.2.1]octane-2-carboxylate (3.2 g, 6.2 mmol) was performed according to general procedure B2. The crude reaction mixture was concentrated under reduced pressure, and the pH was adjusted to pH=7 with sat. NaHCO₃. The residue was purified using method RFPC2 (10% to 50% gradient in 20 min), affording 2-((4-((1S,5R)-2,6-diazabicyclo[3.2.1]octan-6-yl)pyrimidin-5-yl)oxy)-N-ethyl-5-fluoro-N-isopropylbenzamide (2.5 g, 98%) as a yellow solid.

¹H NMR (400 MHz, DMSO-d₆, 300 K) δ 8.45-8.18 (m, 2H), 7.92-7.70 (m, 1H), 7.36-7.14 (m, 2H), 711-6.88 (m, 1H), 4.90-4.14 (m, 2H), 3.92-3.60 (m, 4H), 3.45-3.08 (m, 2H), 2.98-2.87 (m, 1H), 2.87-2.70 (m, 1H), 2.05-1.70 (m, 3H), 1.68-1.48 (m, 1H), 1.28-0.82 (m, 9H). m/z: ES⁺ [M+H]⁺=414.20. N-(2-((4-((1S,5R)-2,6-diazabicyclo[3.2.1]octan-6-yl)pyrimidin-5-yl)oxy)-5-fluorophenyl)-N-ethylisobutyramide

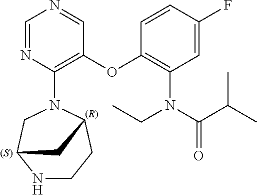

### Step 1: tert-butyl (1S,5R)-6-(5-(2-(N-ethylisobutyramido)-4-fluorophenoxy)pyrimidin-4-yl)-2,6-diazabicyclo[3.2.1]octane-2-carboxylate

The coupling between tert-butyl (1S,5R)-2,6-diazabicyclo[3.2.1]octane-2-carboxylate (226 mg, 1.1 mmol, 0.9 equiv) and N-(2-((4-chloropyrimidin-5-yl)oxy)-5-fluorophenyl)-N-ethylisobutyramide (400 mg, 1.2 mmol, 1.0 equiv) was performed according to general procedure G1. The crude product was then purified using method RFPC1 (10% to 100% gradient in 20 min) to afford tert-butyl (1S,5R)-6-(5-(2-(N-ethylisobutyramido)-4-fluorophenoxy)pyrimidin-4-yl)-2,6-diazabicyclo[3.2.1]octane-2-carboxylate (200 mg, 33%) as a black oil. m/z: ES⁺ [M+H]⁺=514.25.

### Step 2: N-(2-((4-((1S,5R)-2,6-diazabicyclo[3.2.1]octan-6-yl)pyrimidin-5-yl)oxy)-5-fluorophenyl)-N-ethylisobutyramide

The Boc deprotection of tert-butyl (1S,5R)-6-(5-(2-(N-ethylisobutyramido)-4-fluorophenoxy)pyrimidin-4-yl)-2,6-diazabicyclo[3.2.1]octane-2-carboxylate (200 mg, 0.4 mmol) was performed according to general procedure B2. The resulting mixture was concentrated under vacuum to afford crude N-(2-((4-((1S,5R)-2,6-diazabicyclo[3.2.1]octan-6-yl)pyrimidin-5-yl)oxy)-5-fluorophenyl)-N-ethylisobutyramide (140 mg, quant.) as a brown oil. m/z: ES⁺ [M+H]⁺=414.20.

### 2-((4-((1S,5R)-2-((S)-1-(tert-butoxycarbonyl)-2-methylpyrrolidine-2-carbonyl)-2,6-diazabicyclo[3.2.1]octan-6-yl)pyrimidin-5-yl)oxy)-5-fluorobenzoic acid

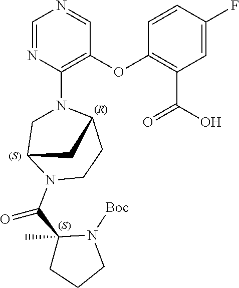

### Step 1: tert-butyl (S)-2-((1S,5R)-6-(5-(4-fluoro-2-(methoxycarbonyl)phenoxy)pyrimidin-4-yl)-2,6-diazabicyclo[3.2.1]octane-2-carbonyl)-2-methylpyrrolidine-1-carboxylate

The coupling between methyl 2-((4-((1S,5R)-2,6-diazabicyclo[3.2.1]octan-6-yl)pyrimidin-5-yl)oxy)-5-fluorobenzoate (300 mg, 0.8 mmol, 1.0 equiv) and (S)-1-(tert-butoxycarbonyl)-2-methylpyrrolidine-2-carboxylic acid (270 mg, 1.2 mmol, 1.4 equiv) was performed according to general procedure A1. The resulting mixture was diluted with water (10 mL) and extracted with EtOAc (3×10 mL). The combined organic layers were washed with brine (10 mL), dried over Na₂SO₄, and concentrated under reduced pressure, to afford. The crude product was then purified using method RFPC1 (10% to 100% gradient in 20 min) to afford tert-butyl (S)-2-((1S,5R)-6-(5-(4-fluoro-2-(methoxycarbonyl)phenoxy)pyrimidin-4-yl)-2,6-diazabicyclo[3.2.1]octane-2-carbonyl)-2-methylpyrrolidine-1-carboxylate (400 mg, 84%) as a yellow solid. m/z: ES⁺ [M+H]⁺=570.25.

### Step 2: 2-((4-((1S,5R)-2-((S)-1-(tert-butoxycarbonyl)-2-methylpyrrolidine-2-carbonyl)-2,6-diazabicyclo[3.2.1]octan-6-yl)pyrimidin-5-yl)oxy)-5-fluorobenzoic acid

A solution of tert-butyl (S)-2-((1S,5R)-6-(5-(4-fluoro-2-(methoxycarbonyl)phenoxy)pyrimidin-4-yl)-2,6-diazabicyclo[3.2.1]octane-2-carbonyl)-2-methylpyrrolidine-1-carboxylate (400 mg, 0.7 mmol, 1.0 equiv) and NaOH (140 mg, 3.5 mmol, 5.0 equiv) in THF (10 mL)/Water (3.5 mL) was stirred at room temperature for 2 h under nitrogen. The resulting mixture was concentrated under reduced pressure, and the residue was acidified to pH 6 with 0.5 M HCl. The resulting mixture was filtered, and the precipitate was washed with water, affording 2-((4-((1S,5R)-2-((S)-1-(tert-butoxycarbonyl)-2-methylpyrrolidine-2-carbonyl)-2,6-diazabicyclo[3.2.1]octan-6-yl)pyrimidin-5-yl)oxy)-5-fluorobenzoic acid (300 mg, 77%) as a colourless solid. m/z: ES⁺ [M+H]⁺=556.25.

### (1S,5R)-6-(5-(2-(2-cyclopropyl-5-methyl-1H-imidazol-1-yl)-4-fluorophenoxy)pyrimidin-4-yl)-2,6-diazabicyclo[3.2.1]octane

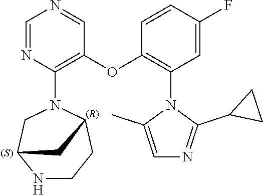

### Step 1: tert-butyl (1S,5R)-6-(5-(2-(2-cyclopropyl-5-methyl-1H-imidazol-1-yl)-4-fluorophenoxy)pyrimidin-4-yl)-2,6-diazabicyclo[3.2.1]octane-2-carboxylate

4-Chloro-5-[2-(2-cyclopropyl-5-methyl-1H-imidazol-1-yl)-4-fluorophenoxy]pyrimidine (380 mg, 1.1 mmol, 1.0 equiv) and tert-butyl (1S,5R)-2,6-diazabicyclo[3.2.1]octane-2-carboxylate (468 mg, 2.2 mmol, 2.0 equiv) were coupled according to general procedure G1. The residue was then purified by Prep-TLC (DCM/MeOH, 20:1) to afford tert-butyl (1S,5R)-6-(5-(2-(2-cyclopropyl-5-methyl-1H-imidazol-1-yl)-4-fluorophenoxy)pyrimidin-4-yl)-2,6-diazabicyclo[3.2.1]octane-2-carboxylate (400 mg, 70%) as a colourless solid. m/z: ES⁺ [M+H]⁺=521.20.

### Step 2: (1S,5R)-6-(5-(2-(2-cyclopropyl-5-methyl-1H-imidazol-1-yl)-4-fluorophenoxy)pyrimidin-4-yl)-2,6-diazabicyclo[3.2.1]octane

The Boc deprotection of tert-butyl (1S,5R)-6-(5-[2-(2-cyclopropyl-5-methylimidazol-1-yl)-4-fluorophenoxy]pyrimidin-4-yl)-2,6-diazabicyclo[3.2.1]octane-2-carboxylate (380 mg, 0.73 mmol) was performed according to general procedure B2. The resulting mixture was concentrated under reduced pressure to afford (1S,5R)-6-(5-(2-(2-cyclopropyl-5-methyl-1H-imidazol-1-yl)-4-fluorophenoxy)pyrimidin-4-yl)-2,6-diazabicyclo[3.2.1]octane (300 mg, 98%) as a colourless solid. The crude product was used in the next step directly without further purification. m/z: ES⁺ [M+H]⁺=421.30.

### (1S,5R)-6-(5-(2-(2-cyclopropyl-5-methyl-1H-imidazol-1-yl)-3,4-difluorophenoxy)pyrimidin-4-yl)-2,6-diazabicyclo[3.2.1]octane

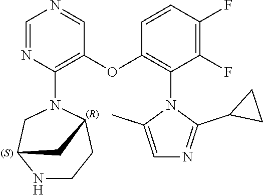

### Step 1: tert-butyl (1S,5R)-6-(5-(2-(2-cyclopropyl-5-methyl-1H-imidazol-1-yl)-3,4-difluorophenoxy)pyrimidin-4-yl)-2,6-diazabicyclo[3.2.1]octane-2-carboxylate

4-Chloro-5-(2-(2-cyclopropyl-5-methyl-1H-imidazol-1-yl)-3,4-difluorophenoxy)pyrimidine (250 mg, 0.69 mmol, 1.0 equiv) and tert-butyl (1S,5R)-2,6-diazabicyclo[3.2.1]octane-2-carboxylate (219 mg, 1.0 mmol, 1.5 equiv) were coupled according to general procedure G1. The residue was purified using method RFPC3 (0% to 100% gradient in 15 min) to afford tert-butyl (1S,5R)-6-(5-(2-(2-cyclopropyl-5-methyl-1H-imidazol-1-yl)-3,4-difluorophenoxy)pyrimidin-4-yl)-2,6-diazabicyclo[3.2.1]octane-2-carboxylate (330 mg, 89%) as a yellow oil. m/z: ES⁺ [M+H]⁺=539.60.

### Step 2: (1S,5R)-6-(5-(2-(2-cyclopropyl-5-methyl-1H-imidazol-1-yl)-3,4-difluorophenoxy)pyrimidin-4-yl)-2,6-diazabicyclo[3.2.1]octane

tert-Butyl (1S,5R)-6-(5-(2-(2-cyclopropyl-5-methyl-1H-imidazol-1-yl)-3,4-difluorophenoxy)pyrimidin-4-yl)-2,6-diazabicyclo[3.2.1]octane-2-carboxylate (330 mg, 0.6 mmol) was deprotected using general procedure B2. The crude product was used in the next step directly without further purification. m/z: ES⁺ [M+H]⁺=439.48.

### (1S,5R)-6-(5-(4-fluoro-2-(2-isopropoxypyridin-3-yl)phenoxy)pyrimidin-4-yl)-2,6-diazabicyclo[3.2.1]octane

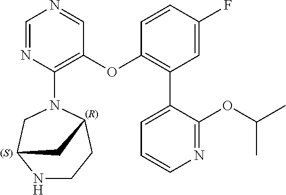

### Step 1: tert-butyl (1S,5R)-6-(5-(4-fluoro-2-(2-isopropoxypyridin-3-yl)phenoxy)pyrimidin-4-yl)-2,6-diazabicyclo[3.2.1]octane-2-carboxylate

A mixture of (2-isopropoxypyridin-3-yl)boronic acid (80 mg, 1.1 mmol, 1.0 equiv), tert-butyl (1S,5R)-6-(5-(2-bromo-4-fluorophenoxy)pyrimidin-4-yl)-2,6-diazabicyclo[3.2.1]octane-2-carboxylate (80 mg, 0.44 mmol, 1.0 equiv), Pd(dppf)Cl₂ (36 mg, 0.044 mmol, 0.1 equiv) and KOAc (129 mg, 1.3 mmol, 3.0 equiv) in dioxane (2 mL) was stirred at room temperature for 2 h under nitrogen. The residue was then purified using method RFPC1 (10% to 50% gradient in 10 min), yielding tert-butyl (1S,5R)-6-(5-(4-fluoro-2-(2-isopropoxypyridin-3-yl)phenoxy)pyrimidin-4-yl)-2,6-diazabicyclo[3.2.1]octane-2-carboxylate (68 mg, 29%) as a brown solid. m/z: ES⁺ [M+H]⁺=535.62.

### Step 2: (1S,5R)-6-(5-(4-fluoro-2-(2-isopropoxypyridin-3-yl)phenoxy)pyrimidin-4-yl)-2,6-diazabicyclo[3.2.1]octane

The Boc deprotection of tert-butyl (1S,5R)-6-(5-(4-fluoro-2-(2-isopropoxypyridin-3-yl)phenoxy)pyrimidin-4-yl)-2,6-diazabicyclo[3.2.1]octane-2-carboxylate (68 mg, 0.13 mmol) was performed according to general procedure B2. The resulting mixture was concentrated under reduced pressure to afford (1S,5R)-6-(5-(4-fluoro-2-(2-isopropoxypyridin-3-yl)phenoxy)pyrimidin-4-yl)-2,6-diazabicyclo[3.2.1]octane (50 mg, 90%) as a brown solid. m/z: ES⁺ [M+H]⁺=435.50.

### (1S,5R)-6-(5-(4-fluoro-2-(2-isopropyl-5-methyl-1H-imidazol-1-yl)phenoxy)pyrimidin-4-yl)-2,6-diazabicyclo[3.2.1]octane

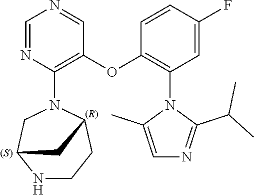

### Step 1: 4-chloro-5-(4-fluoro-2-(2-isopropyl-5-methyl-1H-imidazol-1-yl)phenoxy)pyrimidine

The coupling of 4-chloro-5-(4-fluoro-2-(2-isopropyl-5-methyl-1H-imidazol-1-yl)phenoxy)pyrimidine (230 mg, 0.66 mmol, 1.0 equiv) and tert-butyl (1S,5R)-2,6-diazabicyclo[3.2.1]octane-2-carboxylate (211 mg, 0.99 mmol, 1.5 equiv) was performed according to general procedure G1. The resulting mixture was diluted with water (200 mL) and extracted with EtOAc (3×200 mL), dried over Na₂SO₄, and concentrated under reduced pressure. The crude product was purified using method RPFC1 (10% to 100% gradient in 20 min) to afford tert-butyl (1S,5R)-6-(5-(4-fluoro-2-(2-isopropyl-5-methyl-1H-imidazol-1-yl)phenoxy)pyrimidin-4-yl)-2,6-diazabicyclo[3.2.1]octane-2-carboxylate (250 mg, 72%) as a yellow oil. m/z: ES⁺ [M+H]⁺=523.15.

### Step 2: (1S,5R)-6-(5-(4-fluoro-2-(2-isopropyl-5-methyl-1H-imidazol-1-yl)phenoxy)pyrimidin-4-yl)-2,6-diazabicyclo[3.2.1]octane

The Boc deprotection of tert-butyl (1S,5R)-6-(5-(4-fluoro-2-(2-isopropyl-5-methyl-1H-imidazol-1-yl)phenoxy)pyrimidin-4-yl)-2,6-diazabicyclo[3.2.1]octane-2-carboxylate (240 mg, 0.46 mmol, 1.0 equiv) was performed according to general procedure B2. The reaction was concentrated under reduced pressure to afford (1S,5R)-6-(5-(4-fluoro-2-(2-isopropyl-5-methyl-1H-imidazol-1-yl)phenoxy)pyrimidin-4-yl)-2,6-diazabicyclo[3.2.1]octane (200 mg, HCl salt, crude) as a light yellow oil. m/z: ES⁺ [M+H]⁺=423.15.

### (1S,5R)-6-(5-(2-(3,5-dicyclopropyl-4H-1,2,4-triazol-4-yl)-4-fluorophenoxy)pyrimidin-4-yl)-2,6-diazabicyclo[3.2.1]octane

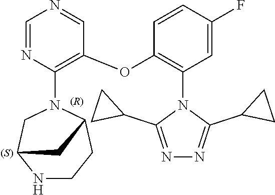

### Step 1: tert-butyl (1S,5R)-6-(5-(2-(3,5-dicyclopropyl-4H-1,2,4-triazol-4-yl)-4-fluoro phenoxy)pyrimidin-4-yl)-2,6-diazabicyclo[3.2.1]octane-2-carboxylate

4-Chloro-5-(2-(3,5-dicyclopropyl-4H-1,2,4-triazol-4-yl)-4-fluorophenoxy)pyrimidine (300 mg, 0.81 mmol, 1.0 equiv) and tert-butyl (1S,5R)-2,6-diazabicyclo[3.2.1]octane-2-carboxylate (188 mg, 0.89 mmol, 1.1 equiv) were coupled according to general procedure G1. The resulting mixture was concentrated under reduced pressure. The residue was purified using method RPFC1 (10% to 100% gradient in 20 min) to afford tert-butyl (1S,5R)-6-(5-(2-(3,5-dicyclopropyl-4H-1,2,4-triazol-4-yl)-4-fluorophenoxy)pyrimidin-4-yl)-2,6-diazabicyclo[3.2.1]octane-2-carboxylate (350 mg, 79%) as an off-white solid. m/z: ES⁺ [M+H]⁺=548.35.

### Step 2: (1S,5R)-6-(5-(2-(3,5-dicyclopropyl-4H-1,2,4-triazol-4-yl)-4-fluorophenoxy)pyrimidin-4-yl)-2,6-diazabicyclo[3.2.1]octane

The Boc deprotection of tert-butyl (1S,5R)-6-(5-(2-(3,5-dicyclopropyl-4H-1,2,4-triazol-4-yl)-4-fluorophenoxy)pyrimidin-4-yl)-2,6-diazabicyclo[3.2.1]octane-2-carboxylate (350 mg, 0.64 mmol, 1.0 equiv) was performed according to general procedure B2. The residue was purified by trituration with EtOAc (5 mL). The precipitate was collected by filtration and washed with EtOAc (3×5 mL) to afford (1S,5R)-6-(5-(2-(3,5-dicyclopropyl-4H-1,2,4-triazol-4-yl)-4-fluorophenoxy)pyrimidin-4-yl)-2,6-diazabicyclo[3.2.1]octane (280 mg, HCl salt, 98%) as a colourless solid. m/z: ES⁺ [M+H]⁺=448.20.

### 6-(2-((4-((1S,5R)-2,6-diazabicyclo[3.2.1]octan-6-yl)pyrimidin-5-yl)oxy)-5-fluorophenyl)-5-isopropyl-1-methylpyridin-2(1H)-one

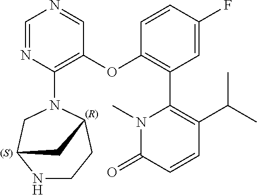

### Step 1: tert-Butyl (1S,5R)-6-(5-(4-fluoro-2-(3-isopropyl-1-methyl-6-oxo-1,6-dihydropyridin-2-yl)phenoxy)pyrimidin-4-yl)-2,6-diazabicyclo[3.2.1]octane-2-carboxylate

The coupling of 6-(2-((4-chloropyrimidin-5-yl)oxy)-5-fluorophenyl)-5-isopropyl-1-methylpyridin-2(1H)-one (400 mg, 1.1 mmol, 1.0 equiv) and tert-butyl (1S,5R)-2,6-diazabicyclo[3.2.1]octane-2-carboxylate (250 mg, 1.2 mmol, 1.1 equiv) was performed according to general procedure G1. The resulting mixture was then concentrated under reduced pressure to give a residue that was purified using method RPFC1 (0% to 80% gradient in 30 min) to afford tert-butyl (1S,5R)-6-(5-(4-fluoro-2-(3-isopropyl-1-methyl-6-oxo-1,6-dihydropyridin-2-yl)phenoxy)pyrimidin-4-yl)-2,6-diazabicyclo[3.2.1]octane-2-carboxylate (500 mg, 85%) as a brown solid. m/z: ES⁺ [M+H]⁺=550.40.

### Step 2: 6-(2-((4-((1S,5R)-2,6-diazabicyclo[3.2.1]octan-6-yl)pyrimidin-5-yl)oxy)-5-fluorophenyl)-5-isopropyl-1-methylpyridin-2(1H)-one

The Boc deprotection of tert-butyl (1S,5R)-6-(5-(4-fluoro-2-(3-isopropyl-1-methyl-6-oxo-1,6-dihydropyridin-2-yl) phenoxy)pyrimidin-4-yl)-2,6-diazabicyclo[3.2.1]octane-2-carboxylate (500 mg, 0.91 mmol, 1.0 equiv) was performed according to general procedure B2. After 1 h the mixture was basified to pH 9 with 1 N NaOH (5 mL), and extracted with DCM (2×30 mL). The combined organic layers were washed with brine (2×50 mL), dried over Na₂SO₄, and concentrated under reduced pressure to afford 6-(2-((4-((1S,5R)-2,6-diazabicyclo[3.2.1]octan-6-yl)pyrimidin-5-yl)oxy)-5-fluorophenyl)-5-isopropyl-1-methylpyridin-2(1H)-one (400 mg, 98%) as a yellow solid. m/z: ES⁺ [M+H]⁺=450.20.

### (1S,5R)-6-(6-(2-(2-cyclopropyl-1H-imidazol-1-yl)-4-fluorophenoxy)-1,2,4-triazin-5-yl)-2,6-diazabicyclo[3.2.1]octane

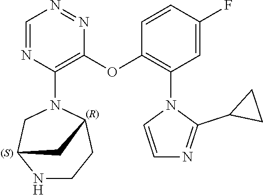

### Step 1: tert-butyl (1S,5R)-6-(6-(2-(2-cyclopropyl-1H-imidazol-1-yl)-4-fluorophenoxy)-1,2,4-triazin-5-yl)-2,6-diazabicyclo[3.2.1]octane-2-carboxylate

The hydrogenolysis of tert-butyl (1S,5R)-6-(3-chloro-6-(2-(2-cyclopropyl-1H-imidazol-1-yl)-4-fluorophenoxy)-1,2,4-triazin-5-yl)-2,6-diazabicyclo[3.2.1]octane-2-carboxylate (340 mg, 0.63 mmol) was performed according to general procedure D1. The resulting mixture was concentrated under reduced pressure, and the residue was purified using method RFPC1 (10% to 100% gradient in 20 min) to afford tert-butyl (1S,5R)-6-(6-(2-(2-cyclopropyl-1H-imidazol-1-yl)-4-fluorophenoxy)-1,2,4-triazin-5-yl)-2,6-diazabicyclo[3.2.1]octane-2-carboxylate (300 mg, 94%) as a light yellow oil. m/z: ES⁺ [M+H]⁺=508.25.

### Step 2: (1S,5R)-6-(6-(2-(2-cyclopropyl-1H-imidazol-1-yl)-4-fluorophenoxy)-1,2,4-triazin-5-yl)-2,6-diazabicyclo[3.2.1]octane

The Boc deprotection of tert-butyl (1S,5R)-6-(6-(2-(2-cyclopropyl-1H-imidazol-1-yl)-4-fluorophenoxy)-1,2,4-triazin-5-yl)-2,6-diazabicyclo[3.2.1]octane-2-carboxylate (290 mg, 0.6 mmol) was performed according to general procedure B2. The resulting mixture was concentrated under vacuum to afford (1S,5R)-6-(6-(2-(2-cyclopropyl-1H-imidazol-1-yl)-4-fluorophenoxy)-1,2,4-triazin-5-yl)-2,6-diazabicyclo[3.2.1]octane (280 mg, crude) as a yellow oil. m/z: ES⁺ [M+H]⁺=408.20.

### (1S,5R)-6-(5-(2-(4-cyclopropylpyridin-3-yl)-4-fluorophenoxy)pyrimidin-4-yl)-2,6-diazabicyclo[3.2.1]octane

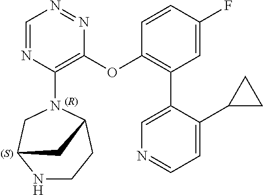

### Step 1: tert-butyl (1S,5R)-6-(5-(2-(4-cyclopropylpyridin-3-yl)-4-fluorophenoxy)pyrimidin-4-yl)-2,6-diazabicyclo[3.2.1]octane-2-carboxylate

A mixture of 4-cyclopropylpyridin-3-ylboronic acid (110 mg, 0.7 mmol, 1.0 equiv), tert-butyl (1S,5R)-6-(5-(2-bromo-4-fluorophenoxy)pyrimidin-4-yl)-2,6-diazabicyclo[3.2.1]octane-2-carboxylate (324 mg, 0.7 mmol, 1.0 equiv), Pd(dppf)Cl₂ (55 mg, 0.07 mmol, 0.1 equiv) and K₂CO₃ (280 mg, 2.0 mmol, 3.0 equiv) in dioxane (2 mL) was stirred at 100° C. for 2 h under nitrogen. The mixture was then diluted with water (200 mL) and extracted with DCM (2×100 mL). The combined organic layers were washed with brine (100 mL), dried over Na₂SO₄ and evaporated to dryness. The residue was then purified using method RFPC1 (10% to 100% gradient in 20 min), yielding tert-butyl (1S,5R)-6-(5-(2-(4-cyclopropylpyridin-3-yl)-4-fluorophenoxy)pyrimidin-4-yl)-2,6-diazabicyclo[3.2.1]octane-2-carboxylate (240 mg, 69%) as a light yellow solid. m/z: ES⁺ [M+H]⁺=518.20.

### Step 2: (1S,5R)-6-(5-(2-(4-cyclopropylpyridin-3-yl)-4-fluorophenoxy)pyrimidin-4-yl)-2,6-diazabicyclo[3.2.1]octane

The Boc deprotection of tert-butyl (1S,5R)-6-(5-(2-(4-cyclopropylpyridin-3-yl)-4-fluorophenoxy)pyrimidin-4-yl)-2,6-diazabicyclo[3.2.1]octane-2-carboxylate (240 mg, 0.5 mmol) was performed according to general procedure B1. The resulting mixture was concentrated under reduced pressure to afford (1S,5R)-6-(5-(2-(4-cyclopropylpyridin-3-yl)-4-fluorophenoxy)pyrimidin-4-yl)-2,6-diazabicyclo[3.2.1]octane (250 mg, quant.) as a brown solid. m/z: ES⁺ [M+H]⁺=418.15.

### (1S,5R)-6-(5-(2-(4-cyclopropylpyrimidin-5-yl)-4-fluorophenoxy)pyrimidin-4-yl)-2,6-diazabicyclo[3.2.1]octane

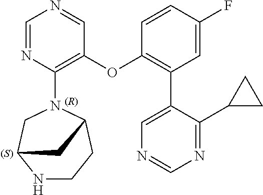

### Step 1: tert-butyl (1S,5R)-6-(5-(2-(4-cyclopropylpyridin-3-yl)-4-fluorophenoxy)pyrimidin-4-yl)-2,6-diazabicyclo[3.2.1]octane-2-carboxylate

A mixture of 4-cyclopropyl-5-(4,4,5,5-tetramethyl-1,3,2-dioxaborolan-2-yl)pyrimidine (123 mg, 0.5 mmol, 1.2 equiv), tert-butyl (1S,5R)-6-(5-(2-bromo-4-fluorophenoxy)pyrimidin-4-yl)-2,6-diazabicyclo[3.2.1]octane-2-carboxylate (200 mg, 0.4 mmol, 1.0 equiv), Pd(dppf)Cl₂ (68 mg, 0.04 mmol, 0.2 equiv) and K₂CO₃ (173 mg, 1.3 mmol, 3.0 equiv) in dioxane (1.6 mL) and water (0.4 mL) was stirred at 100° C. for 2 h under nitrogen. The mixture was then diluted with water (10 mL) and extracted with EtOAc (3×10 mL). The combined organic layers were washed with water (2×20 mL, dried over Na₂SO₄ and evaporated to dryness. The residue was then purified using method RFPC1 (10% to 100% gradient in 30 min), yielding tert-butyl (1S,5R)-6-(5-(2-(4-cyclopropylpyrimidin-5-yl)-4-fluorophenoxy)pyrimidin-4-yl)-2,6-diazabicyclo[3.2.1]octane-2-carboxylate (180 mg, 83%) as alight yellow oil. m/z: ES⁺ [M+H]⁺=519.24.

### Step 2: (1S,5R)-(5-(2-(4-cyclopropylpyrimidin-5-yl)-4-fluorophenoxy)pyrimidin-4-yl)-2,6-diazabicyclo[3.2.1]octane

The Boc deprotection of tert-butyl (1S,5R)-6-(5-(2-(4-cyclopropylpyrimidin-5-yl)-4-fluorophenoxy)pyrimidin-4-yl)-2,6-diazabicyclo[3.2.1]octane-2-carboxylate (180 mg, 0.35 mmol) was performed according to general procedure B1. The resulting mixture was concentrated under reduced pressure to afford (1S,5R)-6-(5-(2-(4-cyclopropylpyrimidin-5-yl)-4-fluorophenoxy)pyrimidin-4-yl)-2,6-diazabicyclo[3.2.1]octane (140 mg, 96%) as a light yellow oil. m/z: ES⁺ [M+H]⁺=419.19.

### (1S,5R)-6-(5-(2-(2-cyclopropyl-1H-imidazol-1-yl)-4-fluorophenoxy)pyrimidin-4-yl)-2,6-diazabicyclo[3.2.1]octane

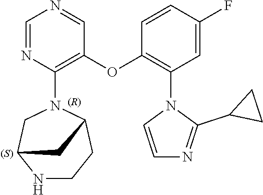

### Step 1: tert-butyl (1S,5R)-6-(5-(2-(2-cyclopropyl-1H-imidazol-1-yl)-4-fluorophenoxy)pyrimidin-4-yl)-2,6-diazabicyclo[3.2.1]octane-2-carboxylate

A mixture of 4-chloro-5-(2-(2-cyclopropyl-1H-imidazol-1-yl)-4-fluorophenoxy)pyrimidine (240 mg, 0.73 mmol, 1 equiv), tert-butyl (1S,5R)-2,6-diazabicyclo[3.2.1]octane-2-carboxylate (308 mg, 1.45 mmol, 2 equiv) and DIEA (375 mg, 2.90 mmol, 4 equiv) in isopropanol (3 mL) was stirred at 80° C. for 1 h under nitrogen. The resulting mixture was concentrated under reduced pressure and purified using method RFPC1 (10% to 80% gradient in 10 min) to afford tert-butyl(1S,5R)-6-(5-(2-(2-cyclopropyl-1H-imidazol-1-yl)-4-fluorophenoxy)pyrimidin-4-yl)-2,6-diazabicyclo[3.2.1]octane-2-carboxylate (300 mg, 81%) as a light yellow semi-solid. (ES, m/z): [M+H]⁺=507.20.

### Step 2: (1S,5R)-6-(5-(2-(2-cyclopropyl-1H-imidazol-1-yl)-4-fluorophenoxy)pyrimidin-4-yl)-2,6-diazabicyclo[3.2.1]octane

The Boc deprotection of tert-butyl (1S,5R)-6-(5-(2-(2-cyclopropyl-1H-imidazol-1-yl)-4-fluorophenoxy)pyrimidin-4-yl)-2,6-diazabicyclo[3.2.1]octane-2-carboxylate (300 mg, 0.6 mmol) was performed according to general procedure B1. The resulting mixture was concentrated under vacuum to afford (1S,5R)-6-(5-(2-(2-cyclopropyl-1H-imidazol-1-yl)-4-fluorophenoxy)pyrimidin-4-yl)-2,6-diazabicyclo[3.2.1]octane (200 mg, 83%) as a yellow oil. (ES, m/z): [M+H]⁺=407.30.

### (1S,5R)-6-(6-(4-fluoro-2-(1-isopropyl-1H-imidazol-5-yl)phenoxy)-1,2,4-triazin-5-yl)-2,6-diazabicyclo[3.2.1]octane

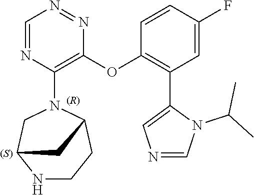

### Step 1: tert-butyl (1S,5R)-6-(6-(4-fluoro-2-(1-isopropyl-1H-imidazol-5-yl)phenoxy)-1,2,4-triazin-5-yl)-2,6-diazabicyclo[3.2.1]octane-2-carboxylate

The hydrogenolysis of tert-butyl (1S,5R)-6-(3-chloro-6-(4-fluoro-2-(1-isopropyl-1H-imidazol-5-yl)phenoxy)-1,2,4-triazin-5-yl)-2,6-diazabicyclo[3.2.1]octane-2-carboxylate (360 mg, 0.7 mmol) was performed according to general procedure D1. The resulting mixture was concentrated under reduced pressure, and the residue was purified using method RFPC1 (10% to 80% gradient in 20 min) to afford tert-butyl (1S,5R)-6-(6-(4-fluoro-2-(1-isopropyl-1H-imidazol-5-yl)phenoxy)-1,2,4-triazin-5-yl)-2,6-diazabicyclo[3.2.1]octane-2-carboxylate (290 mg, 86%) as an off-white solid. m/z: ES⁺ [M+H]⁺=510.25.

### Step 2: (1S,5R)-6-(6-(4-fluoro-2-(1-isopropyl-1H-imidazol-5-yl)phenoxy)-1,2,4-triazin-5-yl)-2,6-diazabicyclo[3.2.1]octane

The Boc deprotection of tert-butyl (1S,5R)-6-(6-(4-fluoro-2-(1-isopropyl-1H-imidazol-5-yl)phenoxy)-1,2,4-triazin-5-yl)-2,6-diazabicyclo[3.2.1]octane-2-carboxylate (250 mg, 0.5 mmol), was performed according to general procedure B2. The resulting mixture was concentrated under vacuum, diluted with water (20 mL), and basified to pH-10 with 2 M NaOH. The resulting mixture was extracted with DCM (2×40 mL). The combined organic layers were washed with brine (20 mL), dried over Na₂SO₄, and concentrated under reduced pressure. The residue was purified by Prep-TLC (DCM/NH₃ in MeOH (10:1)) to afford (1S,5R)-6-(6-(4-fluoro-2-(1-isopropyl-1H-imidazol-5-yl)phenoxy)-1,2,4-triazin-5-yl)-2,6-diazabicyclo[3.2.1]octane (180 mg, 90%) as an off-white solid. m/z: ES⁺ [M+H]⁺=410.25.

### (1S,5R)-6-(6-(2-(4-cyclopropylpyridin-3-yl)-4-fluorophenoxy)-1,2,4-triazin-5-yl)-2,6-diazabicyclo[3.2.1]octane

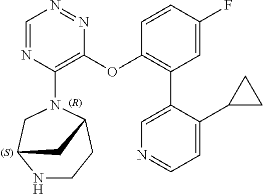

### Step 1: tert-butyl (1S,5R)-6-(6-(2-(4-cyclopropylpyridin-3-yl)-4-fluorophenoxy)-1,2,4-triazin-5-yl)-2,6-diazabicyclo[3.2.1]octane-2-carboxylate

The hydrogenolysis of tert-butyl (1S,5R)-6-(3-chloro-6-(2-(4-cyclopropylpyridin-3-yl)-4-fluorophenoxy)-1,2,4-triazin-5-yl)-2,6-diazabicyclo[3.2.1]octane-2-carboxylate (480 mg, 0.9 mmol) was performed according to general procedure D1. The resulting mixture was quenched with sat. NH₄Cl at 0° C. and extracted with EtOAc (2×30 mL). The combined organic layers were washed with brine (2×20 mL), dried over Na₂SO₄, and concentrated under reduced pressure. The residue was purified using method RFPC1 (40% to 80% gradient in 10 min) to afford tert-butyl (1S,5R)-6-(6-(2-(4-cyclopropylpyridin-3-yl)-4-fluorophenoxy)-1,2,4-triazin-5-yl)-2,6-diazabicyclo[3.2.1]octane-2-carboxylate (400 mg, 89%) as a yellow solid. m/z: ES⁺ [M+H]⁺=519.25.

### Step 2: (1S,5R)-6-(6-(2-(4-cyclopropylpyridin-3-yl)-4-fluorophenoxy)-1,2,4-triazin-5-yl)-2,6-diazabicyclo[3.2.1]octane

The Boc deprotection of tert-butyl (1S,5R)-6-(6-(2-(4-cyclopropylpyridin-3-yl)-4-fluorophenoxy)-1,2,4-triazin-5-yl)-2,6-diazabicyclo[3.2.1]octane-2-carboxylate (380 mg, 0.7 mmol), was performed according to general procedure B2. The resulting mixture was basified to pH-9 with sat NaHC03, and extracted with DCM (2×40 mL).

The combined organic layers were washed with brine (2×30 mL), dried over Na₂SO₄, and concentrated under reduced pressure to afford (1S,5R)-6-(6-(2-(4-cyclopropylpyridin-3-yl)-4-fluorophenoxy)-1,2,4-triazin-5-yl)-2,6-diazabicyclo[3.2.1]octane (300 mg, 98%) as a brown oil. m/z: ES⁺ [M+H]⁺=419.10.

### (1S,5R)-6-(6-(2-(4-cyclopropylpyrimidin-5-yl)-4-fluorophenoxy)-1,2,4-triazin-5-yl)-2,6-diazabicyclo[3.2.1]octane

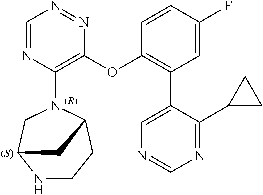

### Step 1: tert-butyl (1S,5R)-6-(6-(2-(4-cyclopropylpyrimidin-5-yl)-4-fluorophenoxy)-1,2,4-triazin-5-yl)-2,6-diazabicyclo[3.2.1]octane-2-carboxylate

The hydrogenolysis of tert-butyl (1S,5R)-6-(3-chloro-6-(2-(4-cyclopropylpyrimidin-5-yl)-4-fluorophenoxy)-1,2,4-triazin-5-yl)-2,6-diazabicyclo[3.2.1]octane-2-carboxylate (600 mg, 1.1 mmol) was performed according to general procedure D1. The resulting mixture was concentrated under reduced pressure, and the residue was purified using method RFPC3 (10% to 100% gradient in 15 min) to afford tert-butyl (1S,5R)-6-(6-(2-(4-cyclopropylpyrimidin-5-yl)-4-fluorophenoxy)-1,2,4-triazin-5-yl)-2,6-diazabicyclo[3.2.1]octane-2-carboxylate (400 mg, 71%) as a yellow solid. m/z: ES⁺ [M+H]⁺=520.05.

### Step 2: (1S,5R)-6-(6-(2-(4-cyclopropylpyrimidin-5-yl)-4-fluorophenoxy)-1,2,4-triazin-5-yl)-2,6-diazabicyclo[3.2.1]octane

The Boc deprotection of tert-butyl (1S,5R)-6-(6-[2-(4-cyclopropylpyrimidin-5-yl)-4-fluorophenoxy]-1,2,4-triazin-5-yl-2,6-diazabicyclo[3.2.1]octane-2-carboxylate (400 mg, 0.8 mmol), was performed according to general procedure B2. The resulting mixture was concentrated under reduced pressure to afford (1S,5R)-6-(6-(2-(4-cyclopropylpyrimidin-5-yl)-4-fluorophenoxy)-1,2,4-triazin-5-yl)-2,6-diazabicyclo[3.2.1]octane (250 mg, crude) as a yellow solid. m/z: ES⁺ [M+H]⁺=420.20.

### (1S,5R)-6-(6-(2-(4,6-dimethylpyrimidin-5-yl)-4-fluorophenoxy)-1,2,4-triazin-5-yl)-2,6-diazabicyclo[3.2.1]octane

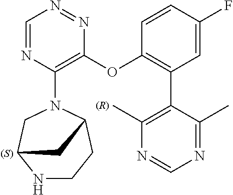

### Step 1: tert-butyl (1S,5R)-6-(3-chloro-6-(2-(4,6-dimethylpyrimidin-5-yl)-4-fluorophenoxy)-1,2,4-triazin-5-yl)-2,6-diazabicyclo[3.2.1]octane-2-carboxylate

The coupling between 2-(4,6-dimethylpyrimidin-5-yl)-4-fluorophenol (400 mg, 1.8 mmol) and tert-butyl (1S,5R)-6-(3,6-dichloro-1,2,4-triazin-5-yl)-2,6-diazabicyclo[3.2.1]octane-2-carboxylate (726 mg, 2.0 mmol) was performed according to general procedure C₁. The resulting mixture was stirred at 80° C. for 2 h, and then concentrated under vacuum. The residue was purified using method RFPC1 (10% to 50% gradient in 10 min) to afford tert-butyl (1S,5R)-6-(3-chloro-6-(2-(4,6-dimethylpyrimidin-5-yl)-4-fluorophenoxy)-1,2,4-triazin-5-yl)-2,6-diazabicyclo[3.2.1]octane-2-carboxylate (800 mg, 81%) as a light brown solid. m/z: ES⁺ [M+H]⁺=542.20.

### Step 2: tert-butyl (1S,5R)-6-(6-(2-(4,6-dimethylpyrimidin-5-yl)-4-fluorophenoxy)-1,2,4-triazin-5-yl)-2,6-diazabicyclo[3.2.1]octane-2-carboxylate

The hydrogenolysis of tert-butyl (1S,5R)-6-(3-chloro-6-(2-(4,6-dimethylpyrimidin-5-yl)-4-fluorophenoxy)-1,2,4-triazin-5-yl)-2,6-diazabicyclo[3.2.1]octane-2-carboxylate (800 mg, 1.5 mmol) was carried out according to general procedure D1. The resulting mixture was diluted with water (50 mL), and extracted with DCM (2×50 mL). The combined organic layers were washed with water (2×100 mL), dried over Na₂SO₄, and concentrated under reduced pressure. The residue was purified using method RFPC1 (10% to 50% gradient in 10 min) to afford tert-butyl (1S,5R)-6-(6-(2-(4,6-dimethylpyrimidin-5-yl)-4-fluorophenoxy)-1,2,4-triazin-5-yl)-2,6-diazabicyclo[3.2.1]octane-2-carboxylate (550 mg, 73%) as a brown solid. m/z: ES⁺ [M+H]⁺=508.24.

### Step 3: (1S,5R)-6-(6-(2-(4,6-dimethylpyrimidin-5-yl)-4-fluorophenoxy)-1,2,4-triazin-5-yl)-2,6-diazabicyclo[3.2.1]octane

The Boc deprotection of tert-butyl (1S,5R)-6-(6-[2-(4,6-dimethylpyrimidin-5-yl)-4-fluorophenoxy]-1,2,4-triazin-5-yl)-2,6-diazabicyclo[3.2.1]octane-2-carboxylate (200 mg, 0.4 mmol, 1.0 equiv) was performed according to general procedure B2, to afford crude (1S,5R)-6-(6-(2-(4,6-dimethylpyrimidin-5-yl)-4-fluorophenoxy)-1,2,4-triazin-5-yl)-2,6-diazabicyclo[3.2.1]octane (150 mg, 93%) as a off-white solid. m/z: ES⁺ [M+H]⁺=408.25. 2-((5-((1S,5R)-2,6-diazabicyclo[3.2.1]octan-6-yl)-1,2,4-triazin-6-yl)oxy)-5-fluoro-N,N-diisopropylbenzamide

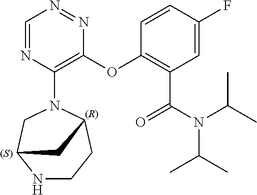

### Step 1: tert-butyl (1S,5R)-6-(3-chloro-6-(2-(diisopropylcarbamoyl)-4-fluorophenoxy)-1,2,4-triazin-5-yl)-2,6-diazabicyclo[3.2.1]octane-2-carboxylate

tert-Butyl (1S,5R)-6-(3-chloro-6-(2-(diisopropylcarbamoyl)-4-fluorophenoxy)-1,2,4-triazin-5-yl)-2,6-diazabicyclo[3.2.1]octane-2-carboxylate was synthesized using 5-fluoro-2-hydroxy-N,N-diisopropylbenzamide (399 mg, 1.7 mmol, 1.5 equiv) according to general procedure C₁. The resulting mixture was concentrated under reduced pressure and the residue was purified using method RFPC3 (10% to 50% gradient in 10 min) to afford tert-butyl (1S,5R)-6-(3-chloro-6-(2-(diisopropylcarbamoyl)-4-fluorophenoxy)-1,2,4-triazin-5-yl)-2,6-diazabicyclo[3.2.1]octane-2-carboxylate (300 mg, 48%.) as acolourless solid. m/z: ES⁺ [M+H]⁺=563.20.

### Step 2: tert-butyl (1S,5R)-6-(6-(2-(diisopropylcarbamoyl)-4-fluorophenoxy)-1,2,4-triazin-5-yl)-2,6-diazabicyclo[3.2.1]octane-2-carboxylate

The hydrogenolysis of tert-butyl (1S,5R)-6-(3-chloro-6-(2-(diisopropylcarbamoyl)-4-fluorophenoxy)-1,2,4-triazin-5-yl)-2,6-diazabicyclo[3.2.1]octane-2-carboxylate (250 mg, 0.36 mmol, 1 equiv) was performed according to general procedure D1. The resulting reaction mixture was diluted with water (50 mL), and extracted with DCM (2×50 mL). The combined organic layers were washed with brine (2×100 mL), dried over Na₂SO₄, and concentrated under reduced pressure. The residue product was purified using method RFPC1 (10% to 50% gradient in 10 min) to afford tert-butyl (1S,5R)-6-(6-(2-(diisopropylcarbamoyl)-4-fluorophenoxy)-1,2,4-triazin-5-yl)-2,6-diazabicyclo[3.2.1]octane-2-carboxylat (200 mg, 70%) as a colourless solid. m/z: ES⁺ [M+H]⁺=529.30.

### Step 3: 2-((5-((1S,5R)-2,6-diazabicyclo[3.2.1]octan-6-yl)-1,2,4-triazin-6-yl)oxy)-5-fluoro-N,N-diisopropylbenzamide

The Boc deprotection of tert-butyl (1S,5R)-6-(6-(2-(diisopropylcarbamoyl)-4-fluorophenoxy)-1,2,4-triazin-5-yl)-2,6-diazabicyclo[3.2.1]octane-2-carboxylat (200 mg, 0.47 mmol, 1.0 equiv) was performed according to general procedure B1. The resulting mixture was quenched with sat. Na₂CO₃ (50 mL), and extracted with DCM (2×50 mL). The combined organic layers were washed with brine (2×100 mL), dried over Na₂SO₄, and concentrated under reduced pressure. The residue was purified using method RFPC1 (10% to 50% gradient in 10 min) to afford 2-((5-((1S,5R)-2,6-diazabicyclo[3.2.1]octan-6-yl)-1,2,4-triazin-6-yl)oxy)-5-fluoro-N,N-diisopropylbenzamide (150 mg, 74%) as a colourless solid. m/z: ES⁺ [M+H]⁺=429.20.

### N-(2-((5-((1S,5R)-2,6-diazabicyclo[3.2.1]octan-6-yl)-1,2,4-triazin-6-yl)oxy)-5-fluorophenyl)-N-ethylcyclopropanecarboxamide

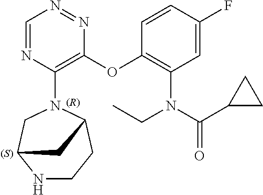

### Step 1: tert-butyl (1S,5R)-6-(6-(2-(N-ethylcyclopropanecarboxamido)-4-fluorophenoxy)-1,2,4-triazin-5-yl)-2,6-diazabicyclo[3.2.1]octane-2-carboxylate

The dechlorination of (1S,5R)-6-(3-chloro-6-(2-(N-ethylcyclopropanecarboxamido)-4-fluorophenoxy)-1,2,4-triazin-5-yl)-2,6-diazabicyclo[3.2.1]octane-2-carboxylate (240 mg, 0.44 mmol, 1.0 equiv) was performed according to general procedure D1. The resulting mixture was diluted with water (10 mL) and extracted with EtOAc (3×10 mL). The combined organic layers were washed with brine (10 mL), dried over Na₂SO₄, and concentrated under reduced pressure. The residue was purified by silica gel column chromatography, eluting with PE/EtOAC (1:1) to afford tert-butyl (1S,5R)-6-(6-(2-(N-ethylcyclopropanecarboxamido)-4-fluorophenoxy)-1,2,4-triazin-5-yl)-2,6-diazabicyclo[3.2.1]octane-2-carboxylate (170 mg, 76%) as alight yellow oil. m/z: ES⁺ [M+H]⁺=513.35.

### Step 2: N-(2-((5-((1S,5R)-2,6-diazabicyclo[3.2.1]octan-6-yl)-1,2,4-triazin-6-yl)oxy)-5-fluorophenyl)-N-ethylcyclopropanecarboxamide

The Boc deprotection of tert-butyl (1S,5R)-6-(6-(2-(N-ethylcyclopropanecarboxamido)-4-fluorophenoxy)-1,2,4-triazin-5-yl)-2,6-diazabicyclo[3.2.1]octane-2-carboxylate (170 mg, 0.33 mmol, 1.0 equiv) was performed according to general procedure B1. The mixture was basified to pH 10 with sat. Na₂CO₃, and extracted with DCM (3×10 mL). The combined organic layers were washed with brine (10 mL), dried over Na₂SO₄, and concentrated under reduced pressure to afford N-(2-((5-((1S,5R)-2,6-diazabicyclo[3.2.1]octan-6-yl)-1,2,4-triazin-6-yl)oxy)-5-fluorophenyl)-N-ethylcyclopropanecarboxamide (130 mg, 95%) as a light yellow oil. m/z: ES⁺ [M+H]⁺=413.25.

### N-(2-((5-((1S,5R)-2,6-diazabicyclo[3.2.1]octan-6-yl)-1,2,4-triazin-6-yl)oxy)-5-fluorophenyl)-N-isopropylcyclopropanecarboxamide

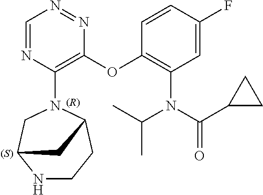

### Step 1: tert-butyl (1S,5R)-6-(6-(4-fluoro-2-(N-isopropylcyclopropane carboxamido)phenoxy)-1,2,4-triazin-5-yl)-2,6-diazabicyclo[3.2.1]octane-2-carboxylate

The dechlorination of tert-butyl (1S,5R)-6-(3-chloro-6-(4-fluoro-2-(N-isopropylcyclopropanecarboxamido)phenoxy)-1,2,4-triazin-5-yl)-2,6-diazabicyclo[3.2.1]octane-2-carboxylate (300 mg, 0.54 mmol, 1.0 equiv) was performed according to general procedure D1. The resulting mixture was diluted with water (10 mL) and extracted with EtOAc (3×10 mL). The combined organic layers were washed with brine (10 mL), dried over Na₂SO₄, and concentrated under reduced pressure. The residue was purified using method RPFC1 (55% to 60% gradient in 10 min) to afford tert-butyl (1S,5R)-6-(6-(4-fluoro-2-(N-isopropylcyclopropanecarboxamido)phenoxy)-1,2,4-triazin-5-yl)-2,6-diazabicyclo[3.2.1]octane-2-carboxylate (200 mg, 71%) as alight yellow oil. m/z: ES⁺ [M+H]⁺=527.40.

### Step 2: N-(2-((5-((1S,5R)-2,6-diazabicyclo[3.2.1]octan-6-yl)-1,2,4-triazin-6-yl)oxy)-5-fluorophenyl)-N-isopropylcyclopropanecarboxamide

The Boc deprotection of tert-butyl (1S,5R)-6-(6-(4-fluoro-2-(N-isopropylcyclopropanecarboxamido)phenoxy)-1,2,4-triazin-5-yl)-2,6-diazabicyclo[3.2.1]octane-2-carboxylate (200 mg, 0.38 mmol, 1.0 equiv) was performed according to general procedure B1. The residue was purified by method RPFC1 (80% to 90% gradient in 10 min) to afford N-(2-((5-((1S,5R)-2,6-diazabicyclo[3.2.1]octan-6-yl)-1,2,4-triazin-6-yl)oxy)-5-fluorophenyl)-N-isopropylcyclopropane carboxamide (150 mg, 93%) as alight yellow oil. m/z: ES⁺ [M+H]⁺=427.10.

### N-(2-((5-((1S,5R)-2,6-Diazabicyclo[3.2.1]octan-6-yl)-1,2,4-triazin-6-yl)oxy)-5-fluorophenyl)-N-cyclopropylcyclopropanecarboxamide

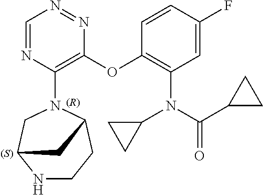

### Step 1: tert-butyl (1S,5R)-6-(6-(2-(N-cyclopropylcyclopropanecarboxamido)-4-fluorophenoxy)-1,2,4-triazin-5-yl)-2,6-diazabicyclo[3.2.1]octane-2-carboxylate

The dechlorination of tert-butyl (1S,5R)-6-(3-chloro-6-(2-(N-cyclopropylcyclopropanecarboxamido)-4-fluorophenoxy)-1,2,4-triazin-5-yl)-2,6-diazabicyclo[3.2.1]octane-2-carboxylate (480 mg, 0.86 mmol, 1.0 equiv) was performed according to general procedure D1. The mixture was diluted with water (100 mL), and extracted with EtOAc (3×30 mL). The combined organic layers were washed with brine (2×20 mL), dried over Na₂SO₄, and concentrated under reduced pressure. The residue was purified using method RPFC1 (0% to 100% gradient in 20 min) to afford tert-butyl (1S,5R)-6-(6-(2-(N-cyclopropylcyclopropanecarboxamido)-4-fluorophenoxy)-1,2,4-triazin-5-yl)-2,6-diazabicyclo[3.2.1]octane-2-carboxylate (300 mg, 67%) as a yellow solid. m/z: ES⁺ [M+H]⁺=525.25.

### Step 2: N-(2-((5-((1S,5R)-2,6-diazabicyclo[3.2.1]octan-6-yl)-1,2,4-triazin-6-yl)oxy)-5-fluorophenyl)-N-cyclopropylcyclopropanecarboxamide

The Boc deprotection of tert-butyl (1S,5R)-6-(6-(2-(N-cyclopropylcyclopropanecarboxamido)-4-fluorophenoxy)-1,2,4-triazin-5-yl)-2,6-diazabicyclo[3.2.1]octane-2-carboxylate (290 mg, 0.55 mmol, 1.0 equiv) was performed according to general procedure B2. The resulting mixture was concentrated under reduced pressure to afford N-(2-((5-((1S,5R)-2,6-diazabicyclo[3.2.1]octan-6-yl)-1,2,4-triazin-6-yl)oxy)-5-fluorophenyl)-N-cyclopropylcyclopropanecarboxamide (250 mg, HCl salt, crude) as a colourless solid. m/z: ES⁺ [M+H]⁺=425.10.

### N-(2-((5-((1S,5R)-2,6-diazabicyclo[3.2.1]octan-6-yl)-1,2,4-triazin-6-yl)oxy)-5-fluorophenyl)-N-isopropylisobutyramide

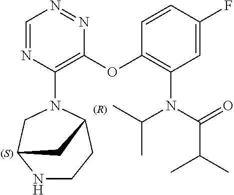

### Step 1: tert-butyl (1S,5R)-6-(6-(4-fluoro-2-(N-isopropylisobutyramido)phenoxy)-1,2,4-triazin-5-yl)-2,6-diazabicyclo[3.2.1]octane-2-carboxylate

The dechlorination of tert-butyl(1S,5R)-6-(3-chloro-6-(4-fluoro-2-(N-isopropylisobutyramido)phenoxy)-1,2,4-triazin-5-yl)-2,6-diazabicyclo[3.2.1]octane-2-carboxylate (330 mg, 0.59 mmol, 1.0 equiv) was performed according to general procedure D1. The resulting mixture was diluted with water (20 mL) and extracted with DCM (3×5 mL). The combined organics were dried over Na₂SO₄ and filtered.

The filtrate was concentrated under reduced pressure and the residue was purified by RPFC1 (10% to 60% gradient in 20 min) to afford tert-butyl (1S,5R)-6-(6-(4-fluoro-2-(N-isopropylisobutyramido)phenoxy)-1,2,4-triazin-5-yl)-2,6-diazabicyclo[3.2.1]octane-2-carboxylate (270 mg, 87%) as a yellow solid. m/z: ES⁺ [M+H]⁺=529.35.

### Step 2: N-(2-((5-((1S,5R)-2,6-diazabicyclo[3.2.1]octan-6-yl)-1,2,4-triazin-6-yl)oxy)-5-fluorophenyl)-N-isopropylisobutyramide

A solution of tert-butyl(1S,5R)-6-(6-(4-fluoro-2-(N-isopropylisobutyramido)phenoxy)-1,2,4-triazin-5-yl)-2,6-diazabicyclo[3.2.1]octane-2-carboxylate (260 mg, 0.49 mmol, 1.0 equiv) was deprotected according to general procedure B2. The resulting mixture was concentrated under reduced pressure and the crude product used in the next step directly without further purification. m/z: ES⁺ [M+H]⁺=429.05.

### (1S,5R)-6-(6-(2-(4-cyclopropyl-6-methylpyrimidin-5-yl)-4-fluorophenoxy)-1,2,4-triazin-5-yl)-2,6-diazabicyclo[3.2.1]octane

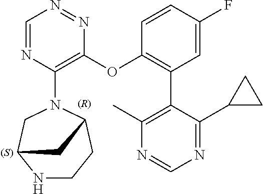

### Step 1: tert-butyl (1S,5R)-6-(6-(2-(4-cyclopropyl-6-methylpyrimidin-5-yl)-4-fluorophenoxy)-1,2,4-triazin-5-yl)-2,6-diazabicyclo[3.2.1]octane-2-carboxylate

The dechlorination of tert-butyl (1S,5R)-6-(3-chloro-6-(2-(4-cyclopropyl-6-methylpyrimidin-5-yl)-4-fluorophenoxy)-1,2,4-triazin-5-yl)-2,6-diazabicyclo[3.2.1]octane-2-carboxylate (900 mg, 1.6 mmol, 1.0 equiv) was performed according to general procedure D1. The reaction was quenched by the addition of water (20 mL) at 0° C., extracted with DCM (3×20 mL), dried over Na₂SO₄, and concentrated under reduced pressure. The residue was purified using method RPFC1 (10% to 100% gradient in 20 min) to afford tert-butyl (1S,5R)-6-(6-(2-(4-cyclopropyl-6-methylpyrimidin-5-yl)-4-fluorophenoxy)-1,2,4-triazin-5-yl)-2,6-diazabicyclo[3.2.1]octane-2-carboxylate (650 mg, 77%) as a yellow solid. m/z: ES⁺ [M+H]⁺=534.26.

### Step 2: (1S,5R)-6-(6-(2-(4-cyclopropyl-6-methylpyrimidin-5-yl)-4-fluorophenoxy)-1,2,4-triazin-5-yl)-2,6-diazabicyclo[3.2.1]octane

The Boc deprotection of tert-butyl (1S,5R)-6-(6-(2-(4-cyclopropyl-6-methylpyrimidin-5-yl)-4-fluorophenoxy)-1,2,4-triazin-5-yl)-2,6-diazabicyclo[3.2.1]octane-2-carboxylate (250 mg, 0.47 mmol, 1.0 equiv) was performed according to general procedure B1. The resulting mixture was concentrated under reduced pressure, and diluted with MeOH (5 mL). The residue was acidified to pH 8 with sat. Na₂CO₃, and extracted with DCM (3×5 mL). The combined organic layers were dried over Na₂SO₄, and concentrated under reduced pressure to afford (1S,5R)-6-(6-(2-(4-cyclopropyl-6-methylpyrimidin-5-yl)-4-fluorophenoxy)-1,2,4-triazin-5-yl)-2,6-diazabicyclo[3.2.1]octane (200 mg, 98%) as a yellow solid. m/z: ES⁺ [M+H]⁺=434.20.

### (1S,5R)-6-(6-(4-fluoro-2-(2-isopropyl-5-methyl-1H-imidazol-1-yl)phenoxy)-1,2,4-triazin-5-yl)-2,6-diazabicyclo[3.2.1]octane

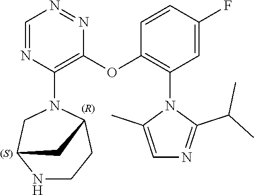

### Step 1: tert-butyl (1S,5R)-6-(6-(4-fluoro-2-(2-isopropyl-5-methyl-1H-imidazol-1-yl)phenoxy)-1,2,4-triazin-5-yl)-2,6-diazabicyclo[3.2.1]octane-2-carboxylate

The dechlorination of tert-butyl (1S,5R)-6-(3-chloro-6-(4-fluoro-2-(2-isopropyl-5-methyl-1H-imidazol-1-yl)phenoxy)-1,2,4-triazin-5-yl)-2,6-diazabicyclo[3.2.1]octane-2-carboxylate (600 mg, 1.1 mmol) was performed according to general procedure D1. The residue was purified using method RFPC1 (10% to 100% gradient in 10 min) to afford tert-butyl (1S,5R)-6-(6-(4-fluoro-2-(2-isopropyl-5-methyl-1H-imidazol-1-yl)phenoxy)-1,2,4-triazin-5-yl)-2,6-diazabicyclo[3.2.1]octane-2-carboxylate (500 mg, 89%) as a light yellow oil. m/z: ES⁺ [M+H]⁺=524.30.

### Step 2: (1S,5R)-6-(6-(4-fluoro-2-(2-isopropyl-5-methyl-1H-imidazol-1-yl)phenoxy)-1,2,4-triazin-5-yl)-2,6-diazabicyclo[3.2.1]octane

The Boc deprotection of tert-butyl (1S,5R)-6-(6-(4-fluoro-2-(2-isopropyl-5-methyl-1H-imidazol-1-yl)phenoxy)-1,2,4-triazin-5-yl)-2,6-diazabicyclo[3.2.1]octane-2-carboxylate (500 mg, 0.96 mmol) was performed according to general procedure B2. The reaction mixture was concentrated under vacuum to give (1S,5R)-6-(5-(4-fluoro-2-(2-isopropyl-5-methyl-1H-imidazol-1-yl)phenoxy)-1,2,4-triazin-5-yl)-2,6-diazabicyclo[3.2.1]octane (450 mg, HCl salt, quant.) as a light brown solid. m/z: ES⁺ [M+H]⁺=424.30.

### (1S,5R)-6-(6-(4-fluoro-2-(2-methoxy-5-methyl-1H-imidazol-1-yl)phenoxy)-1,2,4-triazin-5-yl)-2,6-diazabicyclo[3.2.1]octane

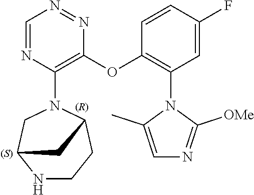

### Step 1: tert-butyl (1S,5R)-6-(6-(4-fluoro-2-(2-methoxy-5-methyl-1H-imidazol-1-yl)phenoxy)-1,2,4-triazin-5-yl)-2,6-diazabicyclo[3.2.1]octane-2-carboxylate

The dechlorination of tert-butyl (1S,5R)-6-(3-chloro-6-(4-fluoro-2-(2-methoxy-5-methyl-1H-imidazol-1-yl)phenoxy)-1,2,4-triazin-5-yl)-2,6-diazabicyclo[3.2.1]octane-2-carboxylate (180 mg, 0.33 mmol, 1.0 equiv) was performed according to general procedure D1. The residue was purified using RPFC1 (10% to 90% gradient in 20 min) to afford tert-butyl (1S,5R)-6-(6-(4-fluoro-2-(2-methoxy-5-methyl-1H-imidazol-1-yl)phenoxy)-1,2,4-triazin-5-yl)-2,6-diazabicyclo[3.2.1]octane-2-carboxylate (140 mg, 83%) as an off-white solid. m/z: ES⁺ [M+H]⁺=512.05.

### Step 2: (1S,5R)-6-(6-(4-fluoro-2-(2-methoxy-5-methyl-1H-imidazol-1-yl)phenoxy)-1,2,4-triazin-5-yl)-2,6-diazabicyclo[3.2.1]octane

The Boc deprotection of tert-butyl (1S,5R)-6-(6-(4-fluoro-2-(2-methoxy-5-methyl-1H-imidazol-1-yl)phenoxy)-1,2,4-triazin-5-yl)-2,6-diazabicyclo[3.2.1]octane-2-carboxylate (140 mg, 0.27 mmol, 1.0 equiv) was performed according to general procedure B2. The reaction mixture was concentrated under vacuum to give (1S,5R)-6-(6-(4-fluoro-2-(2-methoxy-5-methyl-1H-imidazol-1-yl)phenoxy)-1,2,4-triazin-5-yl)-2,6-diaza bicyclo[3.2.1]octane (110 mg, HCl salt, 97%) as a brown solid. m/z: ES⁺ [M+H]⁺=412.00.

### (1S,5R)-6-(6-(4-fluoro-2-(2-isopropoxy-5-methyl-1H-imidazol-1-yl)phenoxy)-1,2,4-triazin-5-yl)-2,6-diazabicyclo[3.2.1]octane

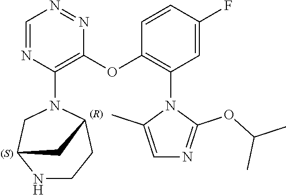

### Step 1: tert-butyl (1S,5R)-6-(6-(4-fluoro-2-(2-isopropoxy-5-methyl-1H-imidazol-1-yl)phenoxy)-1,2,4-triazin-5-yl)-2,6-diazabicyclo[3.2.1]octane-2-carboxylate

The dechlorination of tert-butyl (1S,5R)-6-(3-chloro-6-(4-fluoro-2-(2-isopropoxy-5-methyl-1H-imidazol-1-yl)phenoxy)-1,2,4-triazin-5-yl)-2,6-diazabicyclo[3.2.1]octane-2-carboxylate (210 mg, 0.36 mmol, 1.0 equiv) was performed according to general procedure D1. The residue was purified using RPFC1 (20% to 80% gradient in 30 min) to afford tert-butyl (1S,5R)-6-(6-(4-fluoro-2-(2-isopropoxy-5-methyl-1H-imidazol-1-yl)phenoxy)-1,2,4-triazin-5-yl)-2,6-diazabicyclo[3.2.1]octane-2-carboxylate (120 mg, 60%) as a yellow solid. m/z: ES⁺ [M+H]⁺=540.25.

### Step 2: (1S,5R)-6-(6-(4-fluoro-2-(2-isopropoxy-5-methyl-1H-imidazol-1-yl)phenoxy)-1,2,4-triazin-5-yl)-2,6-diazabicyclo[3.2.1]octane

The Boc deprotection of tert-butyl (1S,5R)-6-(6-(4-fluoro-2-(2-isopropoxy-5-methyl-1H-imidazol-1-yl) phenoxy)-1,2,4-triazin-5-yl)-2,6-diazabicyclo[3.2.1]octane-2-carboxylate (110 mg, 0.19 mmol, 1.0 equiv) was performed according to general procedure B2. The reaction mixture was concentrated under vacuum to give (1S,5R)-6-(6-(4-fluoro-2-(2-isopropoxy-5-methyl-1H-imidazol-1-yl)phenoxy)-1,2,4-triazin-5-yl)-2,6-diazabicyclo[3.2.1]octane (90 mg, HCl salt, crude) as a yellow oil. m/z: ES⁺ [M+H]⁺=440.10.

### (1S,5R)-6-(6-(2-(5-cyclopropyl-2,4-dimethyl-1H-imidazol-1-yl)-4-fluorophenoxy)-1,2,4-triazin-5-yl)-2,6-diazabicyclo[3.2.1]octane

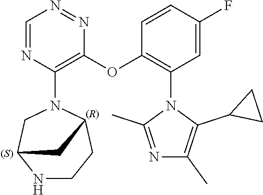

### Step 1: tert-butyl (1S,5R)-6-(6-(2-(5-cyclopropyl-2,4-dimethyl-1H-imidazol-1-yl)-4-fluorophenoxy)-1,2,4-triazin-5-yl)-2,6-diazabicyclo[3.2.1]octane-2-carboxylate

The dechlorination of tert-butyl (1S,5R)-6-(3-chloro-6-(2-(5-cyclopropyl-2,4-dimethyl-1H-imidazol-1-yl)-4-fluoro phenoxy)-1,2,4-triazin-5-yl)-2,6-diazabicyclo[3.2.1]octane-2-carboxylate (130 mg, 0.23 mmol, 1.0 equiv) was performed according to general procedure D2. After work up, (1S,5R)-6-(6-(2-(5-cyclopropyl-2,4-dimethyl-1H-imidazol-1-yl)-4-fluorophenoxy)-1,2,4-triazin-5-yl)-2,6-diazabicyclo[3.2.1]octane-2-carboxylate (110 mg, 90%) was obtained as a yellow semi-solid. m/z: ES⁺ [M+H]⁺=536.20.

### Step 2: (1S,5R)-6-(6-(2-(5-cyclopropyl-2,4-dimethyl-1H-imidazol-1-yl)-4-fluorophenoxy)-1,2,4-triazin-5-yl)-2,6-diazabicyclo[3.2.1]octane

The Boc deprotection of tert-butyl (1S,5R)-6-(6-(2-(5-cyclopropyl-2,4-dimethyl-1H-imidazol-1-yl)-4-fluoro phenoxy)-1,2,4-triazin-5-yl)-2,6-diazabicyclo[3.2.1]octane-2-carboxylate (110 mg, 0.21 mmol, 1.0 equiv) was performed according to general procedure B2. The residue was purified by trituration with EtOAc (5 mL). The precipitate was collected by filtration and washed with EtOAc (3×5 mL) to afford (1S,5R)-6-(6-(2-(5-cyclopropyl-2,4-dimethyl-1H-imidazol-1-yl)-4-fluorophenoxy)-1,2,4-triazin-5-yl)-2,6-diazabicyclo[3.2.1]octane (85 mg, HCl salt, 95%) as an off-white solid. m/z: ES⁺ [M+H]⁺=436.15.

### (1S,5R)-6-(6-(4-fluoro-2-(2-isopropyl-4,5-dimethyl-1H-imidazol-1-yl)phenoxy)-1,2,4-triazin-5-yl)-2,6-diazabicyclo[3.2.1]octane

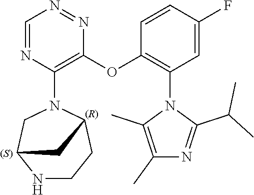

### Step 1: tert-butyl (1S,5R)-6-(6-(4-fluoro-2-(2-isopropyl-4,5-dimethyl-1H-imidazol-1-yl)phenoxy)-1,2,4-triazin-5-yl)-2,6-diazabicyclo[3.2.1]octane-2-carboxylate

The dechlorination of tert-butyl (1S,5R)-6-(3-chloro-6-(4-fluoro-2-(2-isopropyl-4,5-dimethyl-1H-imidazol-1-yl)phenoxy)-1,2,4-triazin-5-yl)-2,6-diazabicyclo[3.2.1]octane-2-carboxylate (2.0 g, 3.5 mmol, 1.0 equiv) was performed according to general procedure D1. The resulting mixture was diluted with water (50 mL), extracted with DCM (3×50 mL), dried over Na₂SO₄, and concentrated under reduced pressure. The residue was purified using method RPFC1 (40% to 70% gradient in 20 min) to afford (1S,5R)-6-(6-(4-fluoro-2-(2-isopropyl-4,5-dimethyl-1H-imidazol-1-yl)phenoxy)-1,2,4-triazin-5-yl)-2,6-diazabicyclo[3.2.1]octane-2-carboxylate (1.1 g, 59%) as a brown solid. m/z: ES⁺ [M+H]⁺=538.30.

### Step 2: (1S,5R)-6-(6-(4-fluoro-2-(2-isopropyl-4,5-dimethyl-1H-imidazol-1-yl)phenoxy)-1,2,4-triazin-5-yl)-2,6-diazabicyclo[3.2.1]octane

The Boc deprotection of tert-butyl (1S,5R)-6-(6-(4-fluoro-2-(2-isopropyl-4,5-dimethyl-1H-imidazol-1-yl)phenoxy)-1,2,4-triazin-5-yl)-2,6-diazabicyclo[3.2.1]octane-2-carboxylate (1.3 g, 2.4 mmol, 1.0 equiv) was performed according to general procedure B2. The crude product was used in the next step directly without further purification. m/z: ES⁺ [M+H]⁺=438.30.

### (1S,5R)-6-(6-(2-(3-cyclopropyl-5-methyl-4H-1,2,4-triazol-4-yl)-4-fluorophenoxy)-1,2,4-triazin-5-yl)-2,6-diazabicyclo[3.2.1]octane

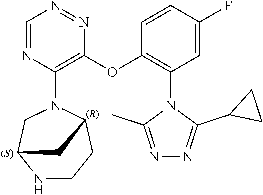

### Step 1: tert-butyl (1S,5R)-6-(6-(2-(3-cyclopropyl-5-methyl-4H-1,2,4-triazol-4-yl)-4-fluorophenoxy)-1,2,4-triazin-5-yl)-2,6-diazabicyclo[3.2.1]octane-2-carboxylate

The dechlorination of tert-butyl (1S,5R)-6-(3-chloro-6-(2-(3-cyclopropyl-5-methyl-4H-1,2,4-triazol-4-yl)-4-fluoro phenoxy)-1,2,4-triazin-5-yl)-2,6-diazabicyclo[3.2.1]octane-2-carboxylate (450 mg, 0.81 mmol, 1.0 equiv) was was performed according to general procedure D1. The residue was purified using method RFPC1 (10% to 100% gradient in 20 min) to afford tert-butyl (1S,5R)-6-(6-(2-(3-cyclopropyl-5-methyl-4H-1,2,4-triazol-4-yl)-4-fluorophenoxy)-1,2,4-triazin-5-yl)-2,6-diazabicyclo[3.2.1]octane-2-carboxylate (240 mg, 57%) as a yellow solid. m/z: ES⁺ [M+H]⁺=523.25.

### Step 2: (1S,5R)-6-(6-(2-(3-cyclopropyl-5-methyl-4H-1,2,4-triazol-4-yl)-4-fluorophenoxy)-1,2,4-triazin-5-yl)-2,6-diazabicyclo[3.2.1]octane

The Boc deprotection of tert-butyl (1S,5R)-6-(6-(2-(3-cyclopropyl-5-methyl-4H-1,2,4-triazol-4-yl)-4-fluorophenoxy)-1,2,4-triazin-5-yl)-2,6-diazabicyclo[3.2.1]octane-2-carboxylate (240 mg, 0.46 mmol, 1.0 equiv) was performed according to general procedure B2. The residue was purified by trituration with EtOAc to afford (1S,5R)-6-(6-(2-(3-cyclopropyl-5-methyl-4H-1,2,4-triazol-4-yl)-4-fluorophenoxy)-1,2,4-triazin-5-yl)-2,6-diazabicyclo[3.2.1]octane (180 mg, HCl salt, 93%) as a yellow solid. m/z: ES⁺ [M+H]⁺=423.35.

### (1S,5R)-6-(6-(2-(3,5-dicyclopropyl-4H-1,2,4-triazol-4-yl)-4-fluorophenoxy)-1,2,4-triazin-5-yl)-2,6-diazabicyclo[3.2.1]octane

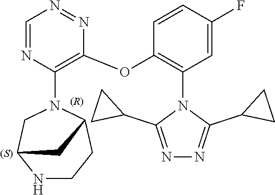

### Step 1: tert-butyl (1S,5R)-6-(6-(2-(3,5-dicyclopropyl-4H-1,2,4-triazol-4-yl)-4-fluorophenoxy)-1,2,4-triazin-5-yl)-2,6-diazabicyclo[3.2.1]octane-2-carboxylate

The dechlorination of tert-butyl (1S,5R)-6-(3-chloro-6-(2-(3,5-dicyclopropyl-4H-1,2,4-triazol-4-yl)-4-fluorophenoxy)-1,2,4-triazin-5-yl)-2,6-diazabicyclo[3.2.1]octane-2-carboxylate (320 mg, 0.55 mmol, 1.0 equiv) was performed according to general procedure D1. The resulting mixture was poured into water and extracted with DCM. The combined organic layers were washed with brine, dried over Na₂SO₄, and concentrated under reduced pressure. The residue was purified by Prep-TLC (DCM/MeOH 10:1) to afford tert-butyl (1S,5R)-6-(6-(2-(3,5-dicyclopropyl-4H-1,2,4-triazol-4-yl)-4-fluorophenoxy)-1,2,4-triazin-5-yl)-2,6-diazabicyclo[3.2.1]octane-2-carboxylate (160 mg, 53%) as a brown solid. m/z: ES⁺ [M+H]⁺=549.50.

### Step 2: (1S,5R)-6-(6-(2-(3,5-dicyclopropyl-4H-1,2,4-triazol-4-yl)-4-fluorophenoxy)-1,2,4-triazin-5-yl)-2,6-diazabicyclo[3.2.1]octane

The Boc deprotection of tert-butyl (1S,5R)-6-(6-(2-(3,5-dicyclopropyl-4H-1,2,4-triazol-4-yl)-4-fluorophenoxy)-1,2,4-triazin-5-yl)-2,6-diazabicyclo[3.2.1]octane-2-carboxylate (160 mg, 0.29 mmol, 1.0 equiv) was performed according to general procedure B2. The residue was purified by trituration with EtOAc (3 mL). The precipitate was collected by filtration and washed with EtOAc (3×3 mL) to afford (1S,5R)-6-(6-(2-(3,5-dicyclopropyl-4H-1,2,4-triazol-4-yl)-4-fluorophenoxy)-1,2,4-triazin-5-yl)-2,6-diazabicyclo[3.2.1]octane (130 mg, HCl salt, 99%) as a colourless solid. m/z: ES⁺ [M+H]⁺=449.05.

### (1S,5R)-6-(6-(2-(3,5-diisopropyl-4H-1,2,4-triazol-4-yl)-4-fluorophenoxy)-1,2,4-triazin-5-yl)-2,6-diazabicyclo[3.2.1]octane

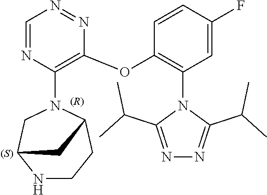

### Step 1: tert-butyl (1S,5R)-6-(6-(2-(3,5-diisopropyl-4H-1,2,4-triazol-4-yl)-4-fluorophenoxy)-1,2,4-triazin-5-yl)-2,6-diazabicyclo[3.2.1]octane-2-carboxylate

The dechlorination of tert-butyl (1S,5R)-6-(3-chloro-6-(2-(3,5-diisopropyl-4H-1,2,4-triazol-4-yl)-4-fluorophenoxy)-1,2,4-triazin-5-yl)-2,6-diazabicyclo[3.2.1]octane-2-carboxylate (600 mg, 1.0 mmol, 1.0 equiv) was performed according to general procedure D1. The resulting mixture was poured into water and extracted with DCM. The combined organic layers were washed with brine, dried over anhydrous Na₂SO₄, and concentrated under reduced pressure. The residue was purified using method RPC1 (10% to 100% gradient in 20 min) to afford tert-butyl (1S,5R)-6-(6-(2-(3,5-diisopropyl-4H-1,2,4-triazol-4-yl)-4-fluorophenoxy)-1,2,4-triazin-5-yl)-2,6-diazabicyclo[3.2.1]octane-2-carboxylate (280 mg, 50%) as a brown solid. m/z: ES⁺ [M+H]⁺=553.35.

### Step 2: (1S,5R)-6-(6-(2-(3,5-diisopropyl-4H-1,2,4-triazol-4-yl)-4-fluorophenoxy)-1,2,4-triazin-5-yl)-2,6-diazabicyclo[3.2.1]octane

The Boc deprotection of tert-butyl (1S,5R)-6-(6-(2-(3,5-diisopropyl-4H-1,2,4-triazol-4-yl)-4-fluorophenoxy)-1,2,4-triazin-5-yl)-2,6-diazabicyclo[3.2.1]octane-2-carboxylate (280 mg, 0.51 mmol, 1.0 equiv) was performed according to general procedure B2. The residue was purified by trituration with EtOAc, and the solid was collected by filtration and washed with EtOAc to afford (1S,5R)-6-(6-(2-(3,5-diisopropyl-4H-1,2,4-triazol-4-yl)-4-fluorophenoxy)-1,2,4-triazin-5-yl)-2,6-diazabicyclo[3.2.1]octane (250 mg, HCl salt, crude) as an off-white solid. m/z: ES⁺ [M+H]⁺=453.25.

### (1S,5R)-6-(6-(2-(4-cyclopropyl-1-methyl-1H-pyrazol-5-yl)-4-fluorophenoxy)-1,2,4-triazin-5-yl)-2,6-diazabicyclo[3.2.1]octane

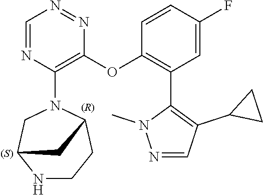

### Step 1: tert-butyl (1S,5R)-6-(6-(2-(4-cyclopropyl-1-methyl-1H-pyrazol-5-yl)-4-fluorophenoxy)-1,2,4-triazin-5-yl)-2,6-diazabicyclo[3.2.1]octane-2-carboxylate

The dechlorination of tert-butyl (1S,5R)-6-(3-chloro-6-(2-(4-cyclopropyl-1-methyl-1H-pyrazol-5-yl)-4-fluorophenoxy)-1,2,4-triazin-5-yl)-2,6-diazabicyclo[3.2.1]octane-2-carboxylate (240 mg, 0.43 mmol, 1.0 equiv) was performed according to general procedure D1. The resulting mixture was diluted with water (50 mL) and extracted with EtOAc (3×200 mL), dried over anhydrous Na₂SO₄. After filtration, the filtrate was concentrated under reduced pressure, and purified using method RPFC1 (10% to 100% gradient in 20 min) to afford tert-butyl (1S,5R)-6-(6-(2-(4-cyclopropyl-1-methyl-1H-pyrazol-5-yl)-4-fluorophenoxy)-1,2,4-triazin-5-yl)-2,6-diazabicyclo[3.2.1]octane-2-carboxylate (200 mg, 89%) as a light brown oil. m/z: ES⁺ [M+H]⁺=522.30.

### Step 2: (1S,5R)-6-(6-(2-(4-cyclopropyl-1-methyl-1H-pyrazol-5-yl)-4-fluorophenoxy)-1,2,4-triazin-5-yl)-2,6-diazabicyclo[3.2.1]octane

The Boc deprotection of tert-butyl (1S,5R)-6-(6-(2-(4-cyclopropyl-1-methyl-1H-pyrazol-5-yl)-4-fluorophenoxy)-1,2,4-triazin-5-yl)-2,6-diazabicyclo[3.2.1]octane-2-carboxylate (190 mg, 0.36 mmol, 1.0 equiv) was performed according to general procedure B2. The resulting mixture was concentrated under reduced pressure to afford (1S,5R)-6-(6-(2-(4-cyclopropyl-1-methyl-1H-pyrazol-5-yl)-4-fluorophenoxy)-1,2,4-triazin-5-yl)-2,6-diazabicyclo[3.2.1]octane (150 mg, crude HCl salt) as a light brown oil. m/z: ES⁺ [M+H]⁺=422.25.

### tert-butyl (1S,5R)-6-(3-chloro-6-(4-fluoro-2-(4-isopropyl-1-methyl-1H-pyrazol-5-yl)phenoxy)-1,2,4-triazin-5-yl)-2,6-diazabicyclo[3.2.1]octane-2-carboxylate

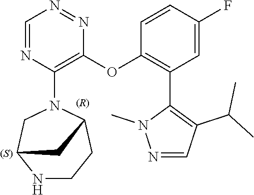

### Step 1: tert-butyl (1S,5R)-6-(6-(4-fluoro-2-(4-isopropyl-1-methyl-1H-pyrazol-5-yl)phenoxy)-1,2,4-triazin-5-yl)-2,6-diazabicyclo[3.2.1]octane-2-carboxylate

The dechlorination of tert-butyl (1S,5R)-6-(3-chloro-6-(4-fluoro-2-(4-isopropyl-1-methyl-1H-pyrazol-5-yl)phenoxy)-1,2,4-triazin-5-yl)-2,6-diazabicyclo[3.2.1]octane-2-carboxylate (250 mg, 0.45 mmol, 1.0 equiv) was performed according to general procedure D1. The resulting mixture was diluted with water (100 mL) and extracted with EtOAc (3×100 mL), dried over Na₂SO₄, and concentrated under reduced pressure. The crude product was purified using method RPFC1 (10% to 100% gradient in 20 min) to afford tert-butyl (1S,5R)-6-(6-(4-fluoro-2-(4-isopropyl-1-methyl-1H-pyrazol-5-yl)phenoxy)-1,2,4-triazin-5-yl)-2,6-diazabicyclo[3.2.1]octane-2-carboxylate (200 mg, 85%) as a light yellow oil. m/z: ES⁺ [M+H]⁺=524.35.

### Step 2: tert-butyl (1S,5R)-6-(3-chloro-6-(4-fluoro-2-(4-isopropyl-1-methyl-1H-pyrazol-5-yl) phenoxy)-1,2,4-triazin-5-yl)-2,6-diazabicyclo[3.2.1]octane-2-carboxylate

The Boc deprotection of tert-butyl (1S,5R)-6-(6-(4-fluoro-2-(4-isopropyl-1-methyl-1H-pyrazol-5-yl)phenoxy)-1,2,4-triazin-5-yl)-2,6-diazabicyclo[3.2.1]octane-2-carboxylate (200 mg, 0.38 mmol, 1.0 equiv) was performed according to general procedure B2. The resulting mixture was concentrated under vacuum to afford (1S,5R)-6-(6-(4-fluoro-2-(4-isopropyl-1-methyl-1H-pyrazol-5-yl)phenoxy)-1,2,4-triazin-5-yl)-2,6-diazabicyclo[3.2.1]octane (150 mg, HCl salt, 76%) as alight yellow oil. m/z: ES⁺ [M+H]⁺=424.25.

### (1S,5R)-6-(6-(2-(4-cyclopropyl-1-isopropyl-1H-pyrazol-5-yl)-4-fluorophenoxy)-1,2,4-triazin-5-yl)-2,6-diazabicyclo[3.2.1]octane

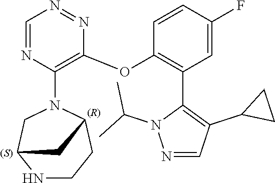

### Step 1: tert-butyl(1S,5R)-6-(6-(2-(4-cyclopropyl-1-isopropyl-1H-pyrazol-5-yl)-4-fluoro phenoxy)-1,2,4-triazin-5-yl)-2,6-diazabicyclo[3.2.1]octane-2-carboxylate

The dechlorination of tert-butyl(1S,5R)-6-(3-chloro-6-(2-(4-cyclopropyl-1-isopropyl-1H-pyrazol-5-yl)-4-fluorophenoxy)-1,2,4-triazin-5-yl)-2,6-diazabicyclo[3.2.1]octane-2-carboxylate (290 mg, 0.49 mmol) was carried out according to general procedure D1. The reaction was quenched by the addition of water (30 mL) and the resulting mixture extracted with EtOAc (2×30 mL). The combined organic layers were washed with brine (60 mL), dried over Na₂SO₄ and filtered. The filtrate was concentrated under reduced pressure and the residue purified by RPFC1 (10% to 90% gradient in 30 min) to afford tert-butyl(1S,5R)-6-(6-(2-(4-cyclopropyl-1-isopropyl-1H-pyrazol-5-yl)-4-fluorophenoxy)-1,2,4-triazin-5-yl)-2,6-diazabicyclo[3.2.1]octane-2-carboxylate (270 mg, 99%) as a yellow solid. m/z: ES⁺ [M+H]+=550.30.

### Step 2: (1S,5R)-6-(6-(2-(4-cyclopropyl-1-isopropyl-1H-pyrazol-5-yl)-4-fluorophenoxy)-1,2,4-triazin-5-yl)-2,6-diazabicyclo[3.2.1]octane

Deprotection of tert-butyl(1S,5R)-6-(6-(2-(4-cyclopropyl-1-isopropyl-1H-pyrazol-5-yl)-4-fluorophenoxy)-1,2,4-triazin-5-yl)-2,6-diazabicyclo[3.2.1]octane-2-carboxylate (270 mg, 0.49 mmol) was carried out according to general procedure B2. This afforded (1S,5R)-6-(6-(2-(4-cyclopropyl-1-isopropyl-1H-pyrazol-5-yl)-4-fluorophenoxy)-1,2,4-triazin-5-yl)-2,6-diazabicyclo[3.2.1]octane (270 mg, HCl salt, crude) as a yellow solid. m/z: ES⁺ [M+H]+=450.30.

### (1S,5R)-6-(6-(4-fluoro-2-(2-isopropyl-1H-imidazol-1-yl)phenoxy)-1,2,4-triazin-5-yl)-2,6-diazabicyclo[3.2.1]octane

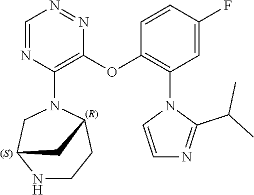

### Step 1: tert-butyl (1S,5R)-6-(6-(4-fluoro-2-(2-isopropyl-1H-imidazol-1-yl)phenoxy)-1,2,4-triazin-5-yl)-2,6-diazabicyclo[3.2.1]octane-2-carboxylate

The hydrogenolysis of tert-butyl (1S,5R)-6-(3-chloro-6-(4-fluoro-2-(2-isopropyl-1H-imidazoll-yl)phenoxy)-1,2,4-triazin-5-yl)-2,6-diazabicyclo[3.2.1]octane-2-carboxylate (150 mg, 0.2 mmol) was performed according to general procedure D1. The residue was purified using method RFPC4 (10% to 50% gradient in 10 min) to afford tert-butyl (1S,5R)-6-(6-(4-fluoro-2-(2-isopropyl-1H-imidazol-1-yl)phenoxy)-1,2,4-triazin-5-yl)-2,6-diazabicyclo[3.2.1]octane-2-carboxylate (130 mg, 91%) as a yellow solid. m/z: ES⁺ [M+H]+=510.26.

### Step 2: (1S,5R)-6-(6-(4-fluoro-2-(2-isopropyl-JH-imidazol-1-yl)phenoxy)-1,2,4-triazin-5-yl)-2,6-diazabicyclo[3.2.1]octane

The Boc deprotection of tert-butyl (1S,5R)-6-(6-(4-fluoro-2-(2-isopropyl-1H-imidazol-1-yl)phenoxy)-1,2,4-triazin 5-yl)-2,6-diazabicyclo[3.2.1]octane-2-carboxylate (130 mg, 0.2 mmol) was performed according to general procedure B2. The residue was purified using method RFPC4 (10% to 50% gradient in 10 min) to afford (1S,5R)-6-(6-(4-fluoro-2-(2-isopropyl-H-imidazol-1-yl)phenoxy)-1,2,4-triazin-5-yl)-2,6-diazabicyclo[3.2.1]octane (100 mg, 92%) as a yellow solid. m/z: ES⁺ [M+H]⁺=410.20.

### (1S,5R)-6-(5-(2-(2-cyclopropyl-5-methyl-1H-imidazol-1-yl)-4-fluorophenoxy)-1,2,4-triazin-5-yl)-2,6-diazabicyclo[3.2.1]octane

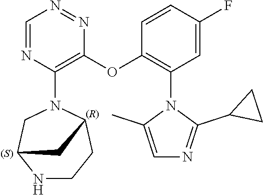

### Step 1: tert-butyl (1S,5R)-6-(6-(2-(2-cyclopropyl-5-methyl-1H-imidazol-1-yl)-4-fluorophenoxy)-1,2,4-triazin-5-yl)-2,6-diazabicyclo[3.2.1]octane-2-carboxylate

The hydrogenolysis of tert-butyl (1S,5R)-6-(3-chloro-6-(2-(2-cyclopropyl-5-methyl-1H-imidazol-1-yl)-4-fluorophenoxy)-1,2,4-triazin-5-yl)-2,6-diazabicyclo[3.2.1]octane-2-carboxylate (100 mg, 0.2 mmol) was performed according to general procedure D1. The residue was purified using method RFPC3 (10% to 50% gradient in 10 min) to afford tert-butyl (1S,5R)-6-(6-(4-fluoro-2-(2-isopropyl-1H-imidazol-1-yl)phenoxy)-1,2,4-triazin-5-yl)-2,6-diazabicyclo[3.2.1]octane-2-carboxylate (80 mg, 85%) as a brown solid. m/z: ES⁺ [M+H]+=522.00.

### Step 2: (1S,5R)-6-(6-(4-fluoro-2-(2-isopropyl-1H-imidazol-1-yl)phenoxy)-1,2,4-triazin-5-yl)-2,6-diazabicyclo[3.2.1]octane

The Boc deprotection of tert-butyl (1S,5R)-6-(6-(2-(2-cyclopropyl-5-methyl-1H-imidazol-1-yl)-4-fluorophenoxy)-1,2,4-triazin-5-yl)-2,6-diazabicyclo[3.2.1]octane-2-carboxylate (280 mg, 0.54 mmol) was performed according to general procedure B1. The reaction mixture was concentrated under vacuum to give (1S,5R)-6-(5-(2-(2-cyclopropyl-5-methyl-1H-imidazol-1-yl)-4-fluorophenoxy)-1,2,4-triazin-5-yl)-2,6-diazabicyclo[3.2.1]octane (220 mg, 97%) as a brown solid. m/z: ES⁺ [M+H]+=422.00.

### N-(2-((5-((1S,5R)-2,6-diazabicyclo[3.2.1]octan-6-yl)-1,2,4-triazin-6-yl)oxy)-5-fluorophenyl)-N-ethylisobutyramide

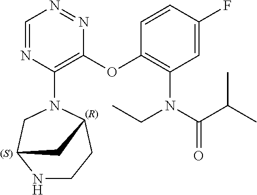

### Step 1: tert-butyl (1S,5R)-6-(6-(2-(N-ethylisobutyramido)-4-fluorophenoxy)-1,2,4-triazin-5-yl)-2,6-diazabicyclo[3.2.1]octane-2-carboxylate

The hydrogenolysis of tert-butyl (1S,5R)-6-(3-chloro-6-(2-(N-ethylisobutyramido)-4-fluorophenoxy)-1,2,4-triazin-5-yl)-2,6-diazabicyclo[3.2.1]octane-2-carboxylate (1.0 g, 2.8 mmol) was performed according to general procedure D1. The residue was purified using method RFPC1 (10% to 100% gradient in 20 min) to afford tert-butyl (1S,5R)-6-(6-(2-(N-ethylisobutyramido)-4-fluorophenoxy)-1,2,4-triazin-5-yl)-2,6-diazabicyclo[3.2.1]octane-2-carboxylate (900 mg, 59%) as a yellow oil. m/z: ES⁺ [M+H]+=549.20.

### Step 2: N-(2-((5-((1S,5R)-2,6-diazabicyclo[3.2.1]octan-6-yl)-1,2,4-triazin-6-yl)oxy)-5-fluorophenyl)-N-ethylisobutyramide

The Boc deprotection of tert-butyl (1S,5R)-6-(6-(2-(N-ethylisobutyramido)-4-fluorophenoxy)-1,2,4-triazin-5-yl)-2,6-diazabicyclo[3.2.1]octane-2-carboxylate (600 mg, 1.6 mmol) was performed according to general procedure B2. The reaction mixture was concentrated under vacuum to give N-(2-((5-((1S,5R)-2,6-diazabicyclo[3.2.1]octan-6-yl)-1,2,4-triazin-6-yl)oxy)-5-fluorophenyl)-N-ethylisobutyramide (450 mg, 93%) as a brown solid. m/z: ES⁺ [M+H]+=415.20.

### 6-(2-((5-((1S,5R)-2,6-diazabicyclo[3.2.1]octan-6-yl)oxy)-5-fluorophenyl)-1-methylpyridin-2-one

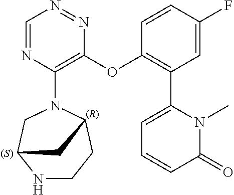

### Step 1: tert-butyl (1S,5R)-6-(6-(4-fluoro-2-(1-methyl-6-oxo-1,6-dihydropyridin-2-yl)phenoxy)-1,2,4-triazin-5-yl)-2,6-diazabicyclo[3.2.1]octane-2-carboxylate

The hydrogenolysis of tert-butyl (1S,5R)-6-(3-chloro-6-[4-fluoro-2-(1-methyl-6-oxopyridin-2-yl)phenoxy]-1,2,4-triazin-5-yl)-2,6-diazabicyclo[3.2.1]octane-2-carboxylate (260 mg, 0.5 mmol, 1.0 equiv) was performed according to general procedure D1. The residue was purified using method RPFC1 (40% to 80% gradient in 10 min) to afford tert-butyl (1S,5R)-6-(6-(4-fluoro-2-(1-methyl-6-oxo-1,6-dihydropyridin-2-yl)phenoxy)-1,2,4-triazin-5-yl)-2,6-diazabicyclo[3.2.1]octane-2-carboxylate (240 mg, 99%) as a yellow solid. m/z: ES⁺ [M+H]+=509.20.

### Step 2: 6-(2-((5-((1S,5R)-2,6-diazabicyclo[3.2.1]octan-6-yl)oxy)-5-fluorophenyl)-1-methylpyridin-2-one

The Boc deprotection of tert-butyl (1S,5R)-6-(6-(4-fluoro-2-(1-methyl-6-oxo-1,6-dihydropyridin-2-yl)phenoxy)-1,2,4-triazin-5-yl)-2,6-diazabicyclo[3.2.1]octane-2-carboxylate (230 mg, 0.5 mmol, 1 equiv) was performed according to general procedure B1. The mixture was basified to pH 9 with saturated NaHCO₃ (aq.) then the resulting mixture was extracted with DCM (2×40 mL). The combined organic layers were washed with brine (1×80 mL), dried over anhydrous Na₂SO₄. After filtration, the filtrate was concentrated under reduced pressure to afford 6-(2-((5-((1S,5R)-2,6-diazabicyclo[3.2.1]octan-6-yl)oxy)-5-fluorophenyl)-1-methylpyridin-2-one (180 mg, 97%) as a yellow oil. m/z: ES⁺ [M+H]+=409.10.

### (1S,5R)-6-(6-(2-(2-cyclopropylpyridin-3-yl)-4-fluorophenoxy)-1,2,4-triazin-5-yl)-2,6-diazabicyclo[3.2.1]octane

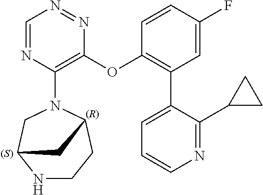

### Step 1: tert-Butyl (1S,5R)-6-(6-(2-(2-cyclopropylpyridin-3-yl)-4-fluorophenoxy)-1,2,4-triazin-5-yl)-2,6-diazabicyclo[3.2.1]octane-2-carboxylate

The hydrogenolysis of tert-butyl (1S,5R)-6-(3-chloro-6-[2-(2-cyclopropylpyridin-3-yl)-4-fluorophenoxy]-1,2,4-triazin-5-yl)-2,6-diazabicyclo[3.2.1]octane-2-carboxylate (100 mg, 0.181 mmol, 1 equiv) was performed according to general procedure D1. The residue was purified using method RPFC4 (10% to 50% gradient in 10 min) to afford in tert-butyl (1S,5R)-6-(6-(2-(2-cyclopropylpyridin-3-yl)-4-fluorophenoxy)-1,2,4-triazin-5-yl)-2,6-diazabicyclo[3.2.1]octane-2-carboxylate (90 mg, 96%) as a yellow solid. m/z: ES⁺ [M+H]+=519.25.

### Step 2: (1S,5R)-6-(6-(2-(2-cyclopropylpyridin-3-yl)-4-fluorophenoxy)-1,2,4-triazin-5-yl)-2,6-diazabicyclo[3.2.1]octane

The Boc deprotection of tert-butyl (1S,5R)-6-(6-[2-(2-cyclopropylpyridin-3-yl)-4-fluorophenoxy]-1,2,4-triazin-5-yl)-2,6-diazabicyclo[3.2.1]octane-2-carboxylate (100 mg, 0.193 mmol, 1 equiv) was performed according to general procedure B2. The residue was purified using method RPFC4 (10% to 50% gradient in 10 min) to afford (1S,5R)-6-(6-(2-(2-cyclopropylpyridin-3-yl)-4-fluorophenoxy)-1,2,4-triazin-5-yl)-2,6-diazabicyclo[3.2.1]octane (80 mg, 99%) as a yellow oil. m/z: ES⁺ [M+H]+=419.35.

### (1S,5R)-6-(6-(2-(5-cyclopropylpyrimidin-4-yl)-4-fluorophenoxy)-1,2,4-triazin-5-yl)-2,6-diazabicyclo[3.2.1]octane

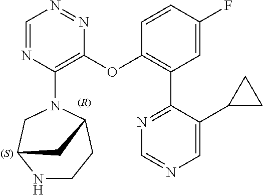

### Step 1: tert-butyl (1S,5R)-6-(6-(2-(5-cyclopropylpyrimidin-4-yl)-4-fluorophenoxy)-1,2,4-triazin-5-yl)-2,6-diazabicyclo[3.2.1]octane-2-carboxylate

The hydrogenolysis of tert-butyl (1S,5R)-6-(3-chloro-6-(2-(5-cyclopropylpyrimidin-4-yl)-4-fluorophenoxy)-1,2,4-triazin-5-yl)-2,6-diazabicyclo[3.2.1]octane-2-carboxylate (310 mg, 0.56 mmol, 1.0 equiv) was performed according to general procedure D1. The residue was purified using method RPFC1 (10% to 100% gradient in 25 min) to afford tert-butyl (1S,5R)-6-(6-(2-(5-cyclopropylpyrimidin-4-yl)-4-fluorophenoxy)-1,2,4-triazin-5-yl)-2,6-diazabicyclo[3.2.1]octane-2-carboxylate (115 mg, 40%) as a dark brown solid. m/z: ES⁺ [M+H]+=520.25.

### Step 2: (1S,5R)-6-(6-(2-(5-cyclopropylpyrimidin-4-yl)-4-fluorophenoxy)-1,2,4-triazin-5-yl)-2,6-diazabicyclo[3.2.1]octane

The Boc deprotection of tert-butyl (1S,5R)-6-(6-(2-(5-cyclopropylpyrimidin-4-yl)-4-fluorophenoxy)-1,2,4-triazin-5-yl)-2,6-diazabicyclo[3.2.1]octane-2-carboxylate (100 mg, 0.192 mmol, 1.0 equiv) was performed according to general procedure B1. The residue was used in the next step without further purification. m/z: ES⁺ [M+H]+=420.25.

### (1S,5R)-6-(6-(2-(2-(tert-butyl)-5-methyl-1H-imidazol-1-yl)-4-fluorophenoxy)-1,2,4-triazin-5-yl)-2,6-diazabicyclo[3.2.1]octane

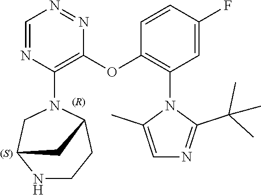

### Step 1: tert-butyl (1S,5R)-6-(6-(2-(2-(tert-butyl)-5-methyl-1H-imidazol-1-yl)-4-fluorophenoxy)-1,2,4-triazin-5-yl)-2,6-diazabicyclo[3.2.1]octane-2-carboxylate

The hydrogenolysis of tert-butyl (1S,5R)-6-(3-chloro-6-(2-(2-cyclopropyl-5-methyl-1H-imidazol-1-yl)-4-fluorophenoxy)-1,2,4-triazin-5-yl)-2,6-diazabicyclo[3.2.1]octane-2-carboxylate (200 mg, 0.35 mmol) was performed according to general procedure D1. The residue was purified using method RFPC1 (10% to 50% gradient in 10 min) to afford tert-butyl (1S,5R)-6-(6-(2-(2-(tert-butyl)-5-methyl-1H-imidazol-1-yl)-4-fluorophenoxy)-1,2,4-triazin-5-yl)-2,6-diazabicyclo[3.2.1]octane-2-carboxylate (130 mg, 69%) as a brown solid. m/z: ES⁺ [M+H]+=538.25.

### Step 2: (1S,5R)-6-(6-(2-(2-(tert-butyl)-5-methyl-1H-imidazol-1-yl)-4-fluorophenoxy)-1,2,4-triazin-5-yl)-2,6-diazabicyclo[3.2.1]octane

The Boc deprotection of tert-butyl (1S,5R)-6-(6-(2-(2-(tert-butyl)-5-methyl-1H-imidazol-1-yl)-4-fluorophenoxy)-1,2,4-triazin-5-yl)-2,6-diazabicyclo[3.2.1]octane-2-carboxylate (130 mg, 0.24 mmol) was performed according to general procedure B1. The reaction mixture was concentrated under vacuum to give (1S,5R)-6-(6-(2-(2-(tert-butyl)-5-methyl-1H-imidazol-1-yl)-4-fluorophenoxy)-1,2,4-triazin-5-yl)-2,6-diazabicyclo[3.2.1]octane (100 mg, 95%) as a yellow solid. m/z: ES⁺ [M+H]+=438.20.

### (1S,5R)-6-(6-(2-(5-cyclopropyl-2-methyl-1H-imidazol-1-yl)-4-fluorophenoxy)-1,2,4-triazin-5-yl)-2,6-diazabicyclo[3.2.1]octane

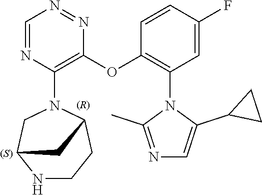

### Step 1: tert-butyl (1S,5R)-6-(6-(2-(5-cyclopropyl-2-methyl-1H-imidazol-1-yl)-4-fluorophenoxy)-1,2,4-triazin-5-yl)-2,6-diazabicyclo[3.2.1]octane-2-carboxylate

The hydrogenolysis of give tert-butyl (1S,5R)-6-(3-chloro-6-(2-(5-cyclopropyl-2-methyl-1H-imidazol-1-yl)-4-fluorophenoxy)-1,2,4-triazin-5-yl)-2,6-diazabicyclo[3.2.1]octane-2-carboxylate (330 mg, 0.6 mmol) was performed according to general procedure D1. The resulting mixture was diluted with water (40 mL) and extracted with DCM (2×40 mL). The combined organic layers were washed with brine (2×80 mL), dried over Na₂SO₄ and concentrated under reduced pressure. The residue was purified using method RPFC1 (30% to 70% gradient in 7 min, Rt=6.1 min), to afford tert-butyl (1S,5R)-6-(6-(2-(5-cyclopropyl-2-methyl-1H-imidazol-1-yl)-4-fluorophenoxy)-1,2,4-triazin-5-yl)-2,6-diazabicyclo[3.2.1]octane-2-carboxylate (220 mg, 71%) as a brown solid. m/z: ES⁺ [M+H]+=522.25.

### Step 2: (1S,5R)-6-(6-(2-(5-cyclopropyl-2-methyl-1H-imidazol-1-yl)-4-fluorophenoxy)-1,2,4-triazin-5-yl)-2,6-diazabicyclo[3.2.1]octane

The Boc deprotection of tert-butyl (1S,5R)-6-(6-(2-(2-cyclopropyl-5-methyl-1H-imidazol-1-yl)-4-fluorophenoxy)-1,2,4-triazin-5-yl)-2,6-diazabicyclo[3.2.1]octane-2-carboxylate (210 mg, 0.4 mmol) was performed according to general procedure B1. The resulting mixture was diluted with sat. NaHCO₃, extracted with DCM (2×50 mL), dried over Na₂SO₄ and concentrated under reduced pressure. This gave crude (1S,5R)-6-(6-(2-(5-cyclopropyl-2-methyl-1H-imidazol-1-yl)-4-fluorophenoxy)-1,2,4-triazin-5-yl)-2,6-diazabicyclo[3.2.1]octane (160 mg, 94%) as a yellow oil. m/z: ES⁺ [M+H]+=422.20.

### (1S,5R)-6-(6-(2-(3-cyclopropylpyrazin-2-yl)-4-fluorophenoxy)-1,2,4-triazin-5-yl)-2,6-diazabicyclo[3.2.1]octane

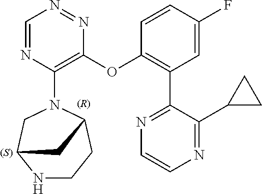

### Step 1: tert-butyl (1S,5R)-6-(6-(2-(3-cyclopropylpyrazin-2-yl)-4-fluorophenoxy)-1,2,4-triazin-5-yl)-2,6-diazabicyclo[3.2.1]octane-2-carboxylate

The hydrogenolysis of tert-butyl (1S,5R)-6-(3-chloro-6-(2-(3-cyclopropylpyrazin-2-yl)-4-fluorophenoxy)-1,2,4-triazin-5-yl)-2,6-diazabicyclo[3.2.1]octane-2-carboxylate (370 mg, 0.7 mmol, 1.0 equiv) was performed according to general procedure D1. The residue was purified using method RPFC3 (40% to 80% gradient in 30 min) to afford tert-butyl (1S,5R)-6-(6-(2-(3-cyclopropylpyrazin-2-yl)-4-fluorophenoxy)-1,2,4-triazin-5-yl)-2,6-diazabicyclo[3.2.1]octane-2-carboxylate (300 mg, 86%) as a colourless solid. m/z: ES⁺ [M+H]+=520.20.

### Step 2: (1S,5R)-6-(6-(2-(3-cyclopropylpyrazin-2-yl)-4-fluorophenoxy)-1,2,4-triazin-5-yl)-2,6-diazabicyclo[3.2.1]octane

The Boc deprotection of tert-butyl (1S,5R)-6-(6-(2-(3-cyclopropylpyrazin-2-yl)-4-fluorophenoxy)-1,2,4-triazin-5-yl)-2,6-diazabicyclo[3.2.1]octane-2-carboxylate (290 mg, 0.6 mmol, 1.0 equiv) was performed according to general procedure B1. The resulting mixture was filtered, then the filter cake was washed with DCM (3×10 mL). The filtrate was concentrated under reduced pressure then the residue was purified using method RPFC3 (40% to 90% gradient in 30 min) to afford (1S,5R)-6-(6-(2-(3-cyclopropylpyrazin-2-yl)-4-fluorophenoxy)-1,2,4-triazin-5-yl)-2,6-diazabicyclo[3.2.1]octane (170 mg, 73%) as a yellow solid. m/z: ES⁺ [M+H]+=420.20.

### (1S,5R)-6-(6-(2-(5-cyclopropyl-2-isopropyl-1H-imidazol-1-yl)-4-fluorophenoxy)-1,2,4-triazin-5-yl)-2,6-diazabicyclo[3.2.1]octane

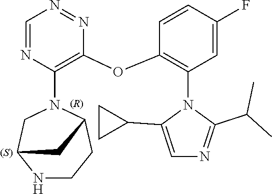

### Step 1: tert-butyl (1S,5R)-6-(6-(2-(5-cyclopropyl-2-isopropyl-1H-imidazol-1-yl)-4-fluorophenoxy)-1,2,4-triazin-5-yl)-2,6-diazabicyclo[3.2.1]octane-2-carboxylate

The hydrogenolysis of tert-butyl (1S,5R)-6-(3-chloro-6-[2-(5-cyclopropyl-2-isopropylimidazol-1-yl)-4-fluorophenoxy]-1,2,4-triazin-5-yl)-2,6-diazabicyclo[3.2.1]octane-2-carboxylate (500 mg, 0.9 mmol) was performed according to general procedure D1. The residue was purified using method RFPC1 (10% to 50% gradient in 10 min) to afford tert-butyl (1S,5R)-6-(6-(2-(5-cyclopropyl-2-isopropyl-1H-imidazol-1-yl)-4-fluorophenoxy)-1,2,4-triazin-5-yl)-2,6-diazabicyclo[3.2.1]octane-2-carboxylate (420 mg, 89%) as a yellow solid. m/z: ES⁺ [M+H]+=550.40.

### Step 2: (1S,5R)-6-(6-(2-(5-cyclopropyl-2-isopropyl-1H-imidazol-1-yl)-4-fluorophenoxy)-1,2,4-triazin-5-yl)-2,6-diazabicyclo[3.2.1]octane

The Boc deprotection of tert-butyl (1S,5R)-6-(6-(2-(5-cyclopropyl-2-isopropyl-1H-imidazol-1-yl)-4-fluorophenoxy)-1,2,4-triazin-5-yl)-2,6-diazabicyclo[3.2.1]octane-2-carboxylate (420 mg, 0.8 mmol) was performed according to general procedure B1. The reaction mixture was concentrated under vacuum to give (1S,5R)-6-(6-(2-(5-cyclopropyl-2-isopropyl-1H-imidazol-1-yl)-4-fluorophenoxy)-1,2,4-triazin-5-yl)-2,6-diazabicyclo[3.2.1]octane (340 mg, 99%) as a yellow solid. m/z: ES⁺ [M+H]+=450.20.

### (1S,5R)-6-(5-(2-(2,5-dicyclopropyl-1H-imidazol-1-yl)-4-fluorophenoxy)pyrimidin-4-yl)-2,6-diazabicyclo[3.2.1]octane

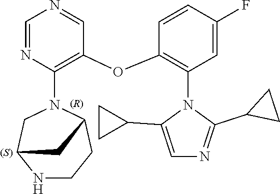

### Step 1: tert-butyl (1S,5R)-6-(5-(2-(2,5-dicyclopropyl-1H-imidazol-1-yl)-4-fluorophenoxy)pyrimidin-4-yl)-2,6-diazabicyclo[3.2.1]octane-2-carboxylate

The coupling between tert-butyl (1S,5R)-2,6-diazabicyclo[3.2.1]octane-2-carboxylate (151 mg, 0.7 mmol, 1.1 equiv) and 4-chloro-5-(2-(2,5-dicyclopropyl-1H-imidazol-1-yl)-4-fluorophenoxy)pyrimidine (240 mg, 0.65 mmol, 1.0 equiv) was performed according to general procedure G1. The resulting mixture was then diluted with water (20 mL), and extracted with EtOAc (3×20 mL). The combined organic layers were washed with brine (2×30 mL), and dried over Na₂SO₄. After filtration, the filtrate was concentrated under reduced pressure, and the residue was purified using method RFPC1 (10% to 100% gradient in 20 min) to afford tert-butyl (1S,5R)-6-(5-(2-(2,5-dicyclopropyl-1H-imidazol-1-yl)-4-fluorophenoxy)pyrimidin-4-yl)-2,6-diazabicyclo[3.2.1]octane-2-carboxylate (200 mg, 57%) as a brown oil. m/z: ES⁺ [M+H]+=547.25.

### Step 2: (1S,5R)-6-(5-(2-(2,5-dicyclopropyl-1H-imidazol-1-yl)-4-fluorophenoxy)pyrimidin-4-yl)-2,6-diazabicyclo[3.2.1]octane

The Boc deprotection of tert-butyl (1S,5R)-6-(5-(2-(2,5-dicyclopropyl-1H-imidazol-1-yl)-4-fluorophenoxy)pyrimidin-4-yl)-2,6-diazabicyclo[3.2.1]octane-2-carboxylate (200 mg, 0.4 mmol) was performed according to general procedure B2, affording crude (1S,5R)-6-(5-(2-(2,5-dicyclopropyl-1H-imidazol-1-yl)-4-fluorophenoxy)pyrimidin-4-yl)-2,6-diazabicyclo[3.2.1]octane (130 mg, 80%) as a yellow oil. m/z: ES⁺ [M+H]+=447.05.

### (1S,5R)-6-(6-(2-(2,5-dicyclopropyl-1H-imidazol-1-yl)-4-fluorophenoxy)-1,2,4-triazin-5-yl)-2,6-diazabicyclo[3.2.1]octane

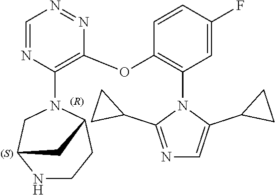

### Step 1: tert-butyl (1S,5R)-6-(6-(2-(2,5-dicyclopropyl-1H-imidazol-1-yl)-4-fluorophenoxy)-1,2,4-triazin-5-yl)-2,6-diazabicyclo[3.2.1]octane-2-carboxylate

The hydrogenolysis of tert-butyl (1S,5R)-6-(3-chloro-6-[2-(5-cyclopropyl-2-isopropylimidazol-1-yl)-4-fluorophenoxy]-1,2,4-triazin-5-yl)-2,6-diazabicyclo[3.2.1]octane-2-carboxylate (2.9 g, 5.0 mmol) was performed according to general procedure D1. The residue was purified using method RFPC1 (10% to 90% gradient in 20 min) to afford tert-butyl (1S,5R)-6-(6-(2-(2,5-dicyclopropyl-1H-imidazol-1-yl)-4-fluorophenoxy)-1,2,4-triazin-5-yl)-2,6-diazabicyclo[3.2.1]octane-2-carboxylate (2.0 g, 73%) as a yellow solid. m/z: ES⁺ [M+H]+=548.20.

### Step 2: (1S,5R)-6-(6-(2-(2,5-dicyclopropyl-1H-imidazol-1-yl)-4-fluorophenoxy)-1,2,4-triazin-5-yl)-2,6-diazabicyclo[3.2.1]octane

The Boc deprotection tert-butyl (1S,5R)-6-(6-(2-(2,5-dicyclopropyl-1H-imidazol-1-yl)-4-fluorophenoxy)-1,2,4-triazin-5-yl)-2,6-diazabicyclo[3.2.1]octane-2-carboxylate (2.0 g, 3.7 mmol) was performed according to general procedure B1. The reaction mixture was concentrated under vacuum to give (1S,5R)-6-(6-(2-(2,5-dicyclopropyl-1H-imidazol-1-yl)-4-fluorophenoxy)-1,2,4-triazin-5-yl)-2,6-diazabicyclo[3.2.1]octane (1.3 g, 80%) as a yellow solid. m/z: ES⁺ [M+H]⁺=448.22.

### 2-((5-((1S,5R)-2,6-diazabicyclo[3.2.1]octan-6-yl)-1,2,4-triazin-6-yl)oxy)-5-fluoro-N-isopropyl-N-methylbenzamide

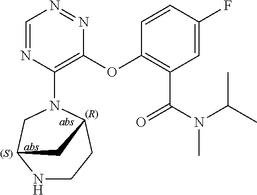

### Step 1: tert-butyl (1S,5R)-6-(6-(4-fluoro-2-(isopropyl(methyl)carbamoyl)phenoxy)-1,2,4-triazin-5-yl)-2,6-diazabicyclo[3.2.1]octane-2-carboxylate

The hydrogenolysis of tert-butyl (1S,5R)-6-(3-chloro-6-(4-fluoro-2-(isopropyl(methyl)carbamoyl)phenoxy)-1,2,4-triazin-5-yl)-2,6-diazabicyclo[3.2.1]octane-2-carboxylate (590 mg, 1.10 mmol, 1 equiv) was performed according to general procedure D1. The residue was purified using method RFPC1 (40% to 80% gradient in 10 min) to afford tert-butyl (1S,5R)-6-(6-(4-fluoro-2-(isopropyl(methyl)carbamoyl)phenoxy)-1,2,4-triazin-5-yl)-2,6-diazabicyclo[3.2.1]octane-2-carboxylate (500 mg, 91%) as alight yellow oil. m/z: ES⁺ [M+H]+=501.25.

### Step 2: 2-((5-((1S,5R)-2,6-diazabicyclo[3.2.1]octan-6-yl)-1,2,4-triazin-6-yl)oxy)-5-fluoro-N-isopropyl-N-methylbenzamide

The Boc deprotection of tert-butyl (1S,5R)-6-(6-(4-fluoro-2-(isopropyl(methyl)carbamoyl)phenoxy)-1,2,4-triazin-5-yl)-2,6-diazabicyclo[3.2.1]octane-2-carboxylate (490 mg, 0.98 mmol) was performed according to general procedure B2. The mixture was basified to pH-9 with sat. NaHCO₃ then the resulting mixture was extracted with DCM (2×40 mL). The combined organic layers were washed with brine (2×30 mL) then dried over Na₂SO₄. After filtration, the filtrate was concentrated under reduced pressure to afford 2-((5-((1S,5R)-2,6-diazabicyclo[3.2.1]octan-6-yl)-1,2,4-triazin-6-yl)oxy)-5-fluoro-N-isopropyl-N-methylbenzamide (370 mg, 94%). m/z: ES⁺ [M+H]+=401.10.

### 4-chloro-5-(4-fluoro-2-(4-isopropyl-1-methyl-1H-1,2,3-triazol-5-yl)phenoxy)pyrimidine

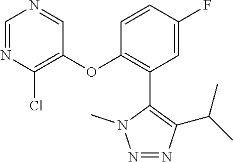 [SMILES: CC(C)c1nnn(C)c1-c1cc(F)ccc1Oc1cncnc1Cl]

### Step 1: 5-(4-fluoro-2-(4-isopropyl-1-methyl-1H-1,2,3-triazol-5-yl)phenoxy)pyrimidine

A solution of 4-fluoro-2-(5-isopropyl-3-methyl-1,2,3-triazol-4-yl)phenol (500 mg, 2.1 mmol, 1.0 equiv), 5-fluoropyrimidine (208 mg, 2.1 mmol, 1.0 equiv) and Cs₂CO₃ (2.1 g, 6.4 mmol, 3.0 equiv) in DMF (5 mL) was stirred at 120° C. for 16 h under nitrogen. The mixture was allowed to cool down to room temperature, diluted with water (40 mL), and extracted with EtOAc (3×30 mL). The combined organic layers were washed with brine (40 mL), dried over anhydrous Na₂SO₄ and concentrated under reduced pressure. The residue was was purified using method RPFC1 (60% to 70% gradient in 30 min), resulting in 5-(4-fluoro-2-(4-isopropyl-1-methyl-1H-1,2,3-triazol-5-yl)phenoxy)pyrimidine (600 mg, 90%) as a light-yellow oil. m/z: ES⁺ [M+H]⁺=314.15.

### Step 2: 5-(4-fluoro-2-(4-isopropyl-1-methyl-1H-1,2,3-triazol-5-yl)phenoxy)pyrimidine 1-oxide

A mixture of 5-(4-fluoro-2-(4-isopropyl-1-methyl-1H-1,2,3-triazol-5-yl)phenoxy)pyrimidine (600 mg, 1.9 mmol, 1.0 equiv) and mCPBA (826 mg, 4.8 mmol, 2.5 equiv) in DCM (6 mL) was stirred at room temperature for 16 h. The reaction was quenched with sat. sodium hyposulfite (aq.) at 0° C. The mixture was basified to pH-9 with sat. Na₂CO₃ (aq.) and extracted with DCM (3×10 mL). The combined organic layers were washed with brine (10 mL), dried over anhydrous Na₂SO₄, and concentrated under reduced pressure. The residue was purified by silica gel column chromatography, eluting with DCM/MeOH (10:1) to afford 5-(4-fluoro-2-(4-isopropyl-1-methyl-1H-1,2,3-triazol-5-yl)phenoxy)pyrimidine 1-oxide (220 mg, 35%) as alight-yellow oil. m/z: ES⁺ [M+H]+=330.15.

### Step 3: 4-chloro-5-(4-fluoro-2-(4-isopropyl-1-methyl-1H-1,2,3-triazol-5-yl)phenoxy)pyrimidine

A solution of 5-(4-fluoro-2-(4-isopropyl-1-methyl-1H-1,2,3-triazol-5-yl)phenoxy)pyrimidine 1-oxide (220 mg, 0.67 mmol, 1.0 equiv) in CHCl₃ (2 mL) was added to a solution of POCl₃ (205 mg, 1.3 mmol, 2.0 equiv) and TFA (152 mg, 1.3 mmol, 2.0 equiv) in CHCl₃ (2 mL), dropwise and at 0° C. The resulting mixture was stirred at 65° C. for 5 h and then basified to pH-9 with sat. Na₂CO₃ (aq.) and extracted with DCM (3×10 mL). The combined organic layers were washed with brine (10 mL), dried over anhydrous Na₂SO₄, and concentrated under reduced pressure. The residue was purified by silica gel column chromatography, eluting with PE/EtOAc (1:1) to afford 4-chloro-5-(4-fluoro-2-(4-isopropyl-1-methyl-1H-1,2,3-triazol-5-yl)phenoxy)pyrimidine (80 mg, 34%) as a light-yellow oil. m/z: ES⁺ [M+H]+=348.10.

### 4-chloro-5-(4-fluoro-2-(2-isopropyl-4,5-dimethyl-1H-imidazol-1-yl)phenoxy)pyrimidine

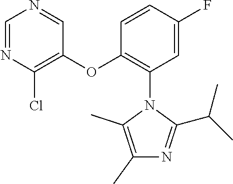 [SMILES: Cc1nc(C(C)C)n(-c2cc(F)ccc2Oc2cncnc2Cl)c1C]

### Step 1: 5-(4-fluoro-2-(2-isopropyl-4,5-dimethyl-1H-imidazol-1-yl)phenoxy)pyrimidine

A solution of 4-fluoro-2-(2-isopropyl-4,5-dimethyl-1H-imidazol-1-yl)phenol (500 mg, 2.1 mmol, 1.0 equiv), 5-fluoropyrimidine (230 mg, 2.3 mmol, 1.1 equiv) and Cs₂CO₃ (1.5 g, 4.7 mmol, 2.0 equiv) in DMF (10 mL) was stirred at 120° C. for 16 h under nitrogen. The mixture was allowed to cool down to room temperature, diluted with water (40 mL), and extracted with EtOAc (3×20 mL). The combined organic layers were washed with brine (2×10 mL), dried over anhydrous Na₂SO₄, and concentrated under reduced pressure. The residue was was purified using method RPFC1 (40% to 70% gradient in 10 min), resulting in 5-(4-fluoro-2-(2-isopropyl-4,5-dimethyl-1H-imidazol-1-yl)phenoxy)pyrimidine (360 mg, 47%) as alight-yellow solid. m/z: ES⁺ [M+H]⁺=327.15.

### Step 2: 5-(4-fluoro-2-(2-isopropyl-4,5-dimethyl-1H-imidazol-1-yl)phenoxy)pyrimidine 1-oxide

A mixture of 5-(4-fluoro-2-(2-isopropyl-4,5-dimethyl-1H-imidazol-1-yl)phenoxy)pyrimidine (360 mg, 1.1 mmol, 1.0 equiv) and urea-hydrogen peroxide (1:1, 260 mg, 2.8 mmol, 2.5 equiv) in THF (5 mL) was stirred at 0° C. for 30 min under nitrogen. TFAA (460 mg, 2.2 mmol, 2.0 equiv) was added, and the resulting mixture was stirred at room temperature for 16 h under nitrogen. The reaction was quenched with sat. sodium hyposulfite (aq.) at 0° C., the mixture was basified to pH-8 with sat. Na₂CO₃ (aq.) and extracted with EtOAc (3×20 mL). The combined organic layers were washed with brine (3×6 mL), dried over anhydrous Na₂SO₄, and concentrated under reduced pressure. This afforded crude 5-(4-fluoro-2-(2-isopropyl-4,5-dimethyl-1H-imidazol-1-yl)phenoxy)pyrimidine 1-oxide (360 mg, 95%) as a yellow solid. m/z: ES⁺ [M+H]⁺=343.15.

### Step 3: 4-chloro-5-(4-fluoro-2-(2-isopropyl-4,5-dimethyl-1H-imidazol-1-yl)phenoxy)pyrimidine

A solution of 5-(4-fluoro-2-(2-isopropyl-4,5-dimethyl-1H-imidazol-1-yl)phenoxy)pyrimidine 1-oxide (360 mg, 1.1 mmol, 1.0 equiv) in POCl₃ (3 mL) was stirred at 110° C. for 2 h under nitrogen. The resulting mixture was diluted with water (5 mL) and extracted with DCM (3×10 mL). The combined organic layers were washed with brine (3 mL), dried over anhydrous Na₂SO₄, and concentrated under reduced pressure. The residue was purified by Prep-TLC, eluting with PE/EtOAc (1:1) to afford 4-chloro-5-(4-fluoro-2-(2-isopropyl-4,5-dimethyl-1H-imidazol-1-yl)phenoxy)pyrimidine (60 mg, 16%) as a yellow oil. m/z: ES⁺ [M+H]⁺=363.10.

### tert-butyl (1S,5R)-6-(3-chloro-6-(2-(2-cyclopropyl-4,5-dimethyl-1H-imidazol-1-yl)-4-fluorophenoxy)-1,2,4-triazin-5-yl)-2,6-diazabicyclo[3.2.1]octane-2-carboxylate

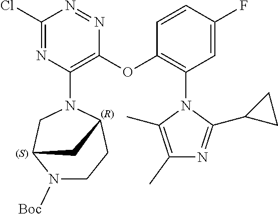

Boc tert-Butyl (1S,5R)-6-(3,6-dichloro-1,2,4-triazin-5-yl)-2,6-diazabicyclo[3.2.1]octane-2-carboxylate (249 mg, 0.69 mmol, 1.0 equiv) and 2-(2-cyclopropyl-4,5-dimethyl-1H-imidazol-1-yl)-4-fluorophenol (170 mg, 0.69 mmol, 1.0 equiv) were coupled according to general procedure C₁. The residue was purified using method RFPC1 (50% to 70% gradient in 10 min) to afford tert-butyl (1S,5R)-6-(3-chloro-6-(2-(2-cyclopropyl-4,5-dimethyl-1H-imidazol-1-yl)-4-fluorophenoxy)-1,2,4-triazin-5-yl)-2,6-diazabicyclo[3.2.1]octane-2-carboxylate (165 mg, 42%) as a yellow solid. m/z: ES⁺ [M+H]⁺=570.23.

### tert-butyl (1S,5R)-6-(3-chloro-6-(4-fluoro-2-(3-isopropyl-1-methyl-6-oxo-1,6-dihydropyridin-2-yl)phenoxy)-1,2,4-triazin-5-yl)-2,6-diazabicyclo[3.2.1]octane-2-carboxylate

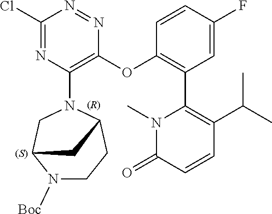

tert-Butyl (1S,5R)-6-(3,6-dichloro-1,2,4-triazin-5-yl)-2,6-diazabicyclo[3.2.1]octane-2-carboxylate (276 mg, 0.77 mmol, 1.0 equiv) and 6-(5-fluoro-2-hydroxyphenyl)-5-isopropyl-1-methylpyridin-2(1H)-one (200 mg, 0.77 mmol, 1.0 equiv) were coupled according to general procedure C₁. The residue was purified using method RFPC1 (30% to 80% gradient in 20 min) to afford tert-butyl (1S,5R)-6-(3-chloro-6-(4-fluoro-2-(3-isopropyl-1-methyl-6-oxo-1,6-dihydropyridin-2-yl)phenoxy)-1,2,4-triazin-5-yl)-2,6-diazabicyclo[3.2.1]octane-2-carboxylate (310 mg, 69%) as a yellow oil. m/z: ES⁺ [M+H]⁺=585.08.

### tert-butyl (1S,5R)-6-(3-chloro-6-(4-fluoro-2-(4-isopropyl-1-methyl-1H-1,2,3-triazol-5-yl)phenoxy)-1,2,4-triazin-5-yl)-2,6-diazabicyclo[3.2.1]octane-2-carboxylate

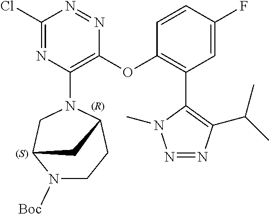

tert-Butyl (1S,5R)-6-(3,6-dichloro-1,2,4-triazin-5-yl)-2,6-diazabicyclo[3.2.1]octane-2-carboxylate (200 mg, 0.56 mmol, 1.0 equiv) and 4-fluoro-2-(4-isopropyl-1-methyl-1H-1,2,3-triazol-5-yl)phenol (130 mg, 0.56 mmol, 1.0 equiv) were coupled according to general procedure C₁. The residue was purified by silica gel column chromatography, eluting with DCM/MeOH (7:1) to afford tert-butyl (1S,5R)-6-(3-chloro-6-(4-fluoro-2-(4-isopropyl-1-methyl-1H-1,2,3-triazol-5-yl)phenoxy)-1,2,4-triazin-5-yl)-2,6-diazabicyclo[3.2.1]octane-2-carboxylate (230 mg, 74%) as a light-yellow oil. m/z: ES⁺ [M+H]⁺=559.30.

### tert-butyl (1S,5R)-6-(3-chloro-6-(2-(cyclopropyl(ethyl)carbamoyl)-4-fluorophenoxy)-1,2,4-triazin-5-yl)-2,6-diazabicyclo[3.2.1]octane-2-carboxylate

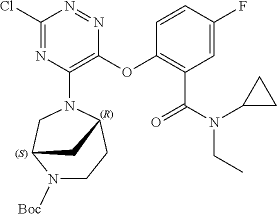

tert-Butyl (1S,5R)-6-(3,6-dichloro-1,2,4-triazin-5-yl)-2,6-diazabicyclo[3.2.1]octane-2-carboxylate (855 mg, 2.4 mmol, 1.0 equiv) and N-cyclopropyl-N-ethyl-5-fluoro-2-hydroxybenzamide (530 mg, 2.4 mmol, 1.0 equiv) were coupled according to general procedure C₁. The residue was purified using method RFPC1 (30% to 80% gradient in 20 min) to afford tert-butyl (1S,5R)-6-(3-chloro-6-(2-(cyclopropyl(ethyl)carbamoyl)-4-fluorophenoxy)-1,2,4-triazin-5-yl)-2,6-diazabicyclo[3.2.1]octane-2-carboxylate (900 mg, 69%) as a yellow solid. m/z: ES⁺ [M+H]⁺=547.03.

### tert-butyl (1S,5R)-6-(3-chloro-6-(4-fluoro-2-(4-isopropyl-6-methylpyrimidin-5-yl)phenoxy)-1,2,4-triazin-5-yl)-2,6-diazabicyclo[3.2.1]octane-2-carboxylate

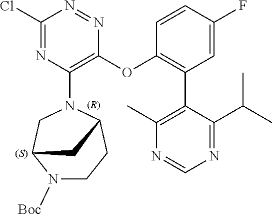

tert-Butyl (1S,5R)-6-(3,6-dichloro-1,2,4-triazin-5-yl)-2,6-diazabicyclo[3.2.1]octane-2-carboxylate (322 mg, 0.89 mmol, 1.1 equiv) and 4-fluoro-2-(4-isopropyl-6-methylpyrimidin-5-yl)phenol (200 mg, 0.81 mmol, 1.0 equiv) were coupled according to general procedure C₁. The resulting mixture was diluted with water (10 mL) and extracted with EtOAc (2×10 mL), dried over Na₂SO₄, and concentrated under reduced pressure. The residue was purified using method RPFC1 (10% to 100% gradient in 20 min) to afford tert-butyl (1S,5R)-6-(3-chloro-6-(4-fluoro-2-(4-isopropyl-6-methylpyrimidin-5-yl)phenoxy)-1,2,4-triazin-5-yl)-2,6-diazabicyclo[3.2.1]octane-2-carboxylate (400 mg, 86%) as a yellow solid. m/z: ES⁺ [M+H]⁺=570.23.

### N-(2-((5-((1S,5R)-2,6-diazabicyclo[3.2.1]octan-6-yl)-3-chloro-1,2,4-triazin-6-yl)oxy)-5-fluorophenyl)-N-benzylisobutyramide

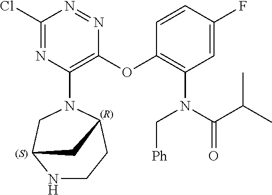

### Step 1: tert-butyl (1S,5R)-6-(3-chloro-6-(2-(N-benzylisobutyramido)-4-fluorophenoxy)-1,2,4-triazin-5-yl)-2,6-diazabicyclo[3.2.1]octane-2-carboxylate

tert-Butyl (1S,5R)-6-(3,6-dichloro-1,2,4-triazin-5-yl)-2,6-diazabicyclo[3.2.1]octane-2-carboxylate (1.5 g, 4.2 mmol, 1.0 equiv) and N-benzyl-N-(5-fluoro-2-hydroxyphenyl)isobutyramide (1.3 g, 4.6 mmol, 1.1 equiv) were coupled according to general procedure C₁. The resulting mixture was diluted with water (50 mL) and extracted with EtOAc (2×50 mL). The combined organic layers were washed with water (2×50 mL), dried over Na₂SO₄, and concentrated under reduced pressure. The residue was purified using method RPFC1 (10% to 100% gradient in 20 min) to afford tert-butyl (1S,5R)-6-(3-chloro-6-(2-(N-benzylisobutyramido)-4-fluorophenoxy)-1,2,4-triazin-5-yl)-2,6-diazabicyclo[3.2.1]octane-2-carboxylate (1.2 g, 47%) as a yellow solid. m/z: ES⁺ [M+H]⁺=611.30.

### Step 2: N-(2-((5-((1S,5R)-2,6-diazabicyclo[3.2.1]octan-6-yl)-3-chloro-1,2,4-triazin-6-yl)oxy)-5-fluorophenyl)-N-benzylisobutyramide

The Boc deprotection of tert-butyl (1S,5R)-6-(3-chloro-6-(2-(N-benzylisobutyramido)-4-fluorophenoxy)-1,2,4-triazin-5-yl)-2,6-diazabicyclo[3.2.1]octane-2-carboxylate (50 mg, 0.09 mmol, 1.0 equiv) was performed according to general procedure B1. The mixture was concentrated under reduced pressure to give N-(2-((5-((1S,5R)-2,6-diazabicyclo[3.2.1]octan-6-yl)-3-chloro-1,2,4-triazin-6-yl)oxy)-5-fluorophenyl)-N-benzylisobutyramide (HCl salt, 300 mg, 81%) as a light-yellow solid. m/z: ES⁺ [M+H]⁺=511.20.

### tert-butyl (1S,5R)-6-(3-chloro-6-(2-(ethyl(3-isopropyloxetan-3-yl)amino)-4-fluorophenoxy)-1,2,4-triazin-5-yl)-2,6-diazabicyclo[3.2.1]octane-2-carboxylate

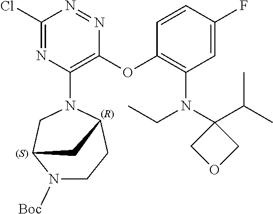

tert-Butyl (1S,5R)-6-(3,6-dichloro-1,2,4-triazin-5-yl)-2,6-diazabicyclo[3.2.1]octane-2-carboxylate (180 mg, 0.50 mmol, 0.9 equiv) and 2-(ethyl(3-isopropyloxetan-3-yl)amino)-4-fluorophenol (140 mg, 0.55 mmol, 1.0 equiv) were coupled according to general procedure C₁. The residue was purified using method RFPC1 (30% to 70% gradient in 20 min) to afford tert-butyl (1S,5R)-6-(3-chloro-6-(2-(ethyl(3-isopropyloxetan-3-yl)amino)-4-fluorophenoxy)-1,2,4-triazin-5-yl)-2,6-diazabicyclo[3.2.1]octane-2-carboxylate (110 mg, 35%) as a yellow solid. m/z: ES⁺ [M+H]⁺=577.30.

### (1S,5R)-6-(5-(4-fluoro-2-(4-isopropyl-1-methyl-1H-1,2,3-triazol-5-yl)phenoxy)pyrimidin-4-yl)-2,6-diazabicyclo[3.2.1]octane

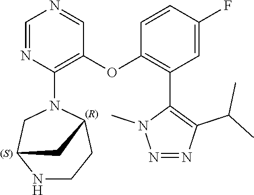

### Step 1: tert-butyl (1S,5R)-6-(5-(4-fluoro-2-(4-isopropyl-1-methyl-1H-1,2,3-triazol-5-yl)phenoxy)pyrimidin-4-yl)-2,6-diazabicyclo[3.2.1]octane-2-carboxylate

The coupling of 4-chloro-5-(4-fluoro-2-(4-isopropyl-1-methyl-1H-1,2,3-triazol-5-yl)phenoxy)pyrimidine (80 mg, 0.23 mmol, 1.0 equiv) and tert-butyl (1S,5R)-2,6-diazabicyclo[3.2.1]octane-2-carboxylate (53 mg, 0.25 mmol, 1.1 equiv) was performed according to general procedure G1. The resulting mixture was diluted with water (10 mL) and extracted with DCM (3×10 mL), dried over Na₂SO₄, and concentrated under reduced pressure. The crude product was purified using silica gel column chromatography, eluting with PE/EtOAc (1:1) to afford tert-butyl (1S,5R)-6-(5-(4-fluoro-2-(4-isopropyl-1-methyl-1H-1,2,3-triazol-5-yl)phenoxy)pyrimidin-4-yl)-2,6-diazabicyclo[3.2.1]octane-2-carboxylate (90 mg, 75%) as a light-yellow oil. m/z: ES⁺ [M+H]⁺=524.35.

### Step 2: (1S,5R)-6-(5-(4-fluoro-2-(2-isopropyl-5-methyl-1H-imidazol-1-yl)phenoxy)pyrimidin-4-yl)-2,6-diazabicyclo[3.2.1]octane

The Boc deprotection of tert-butyl (1S,5R)-6-(5-(4-fluoro-2-(4-isopropyl-1-methyl-1H-1,2,3-triazol-5-yl)phenoxy)pyrimidin-4-yl)-2,6-diazabicyclo[3.2.1]octane-2-carboxylate (90 mg, 0.17 mmol, 1.0 equiv) was performed according to general procedure B2. The reaction was concentrated under reduced pressure to afford (1S,5R)-6-(5-(4-fluoro-2-(4-isopropyl-1-methyl-1H-1,2,3-triazol-5-yl)phenoxy)pyrimidin-4-yl)-2,6-diazabicyclo[3.2.1]octane (85 mg, HCl salt, crude) as a light-yellow solid. m/z: ES⁺ [M+H]⁺=424.25.

### (1S,5R)-6-(5-(4-fluoro-2-(2-isopropyl-4,5-dimethyl-1H-imidazol-1-yl)phenoxy)pyrimidin-4-yl)-2,6-diazabicyclo[3.2.1]octane

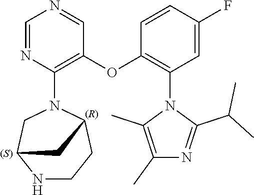

### Step 1: tert-butyl (1S,5R)-6-(5-(4-fluoro-2-(2-isopropyl-4,5-dimethyl-1H-imidazol-1-yl)phenoxy)pyrimidin-4-yl)-2,6-diazabicyclo[3.2.1]octane-2-carboxylate

The coupling of 4-chloro-5-(4-fluoro-2-(2-isopropyl-4,5-dimethyl-1H-imidazol-1-yl)phenoxy)pyrimidine (60 mg, 0.17 mmol, 1.0 equiv) and tert-butyl (1S,5R)-2,6-diazabicyclo[3.2.1]octane-2-carboxylate (35 mg, 0.17 mmol, 1.0 equiv) was performed according to general procedure G1. The resulting mixture was concentrated under reduced pressure and the residue was purified using method RPFC1 (10% to 50% gradient in 10 min), to afford tert-butyl (1S,5R)-6-(5-(4-fluoro-2-(2-isopropyl-4,5-dimethyl-1H-imidazol-1-yl)phenoxy)pyrimidin-4-yl)-2,6-diazabicyclo[3.2.1]octane-2-carboxylate (60 mg, 67%) as a yellow solid. m/z: ES⁺ [M+H]⁺=537.35.

### Step 2: (1S,5R)-6-(5-(4-fluoro-2-(2-isopropyl-4,5-dimethyl-1H-imidazol-1-yl)phenoxy)pyrimidin-4-yl)-2,6-diazabicyclo[3.2.1]octane

The Boc deprotection of tert-butyl (1S,5R)-6-(5-(4-fluoro-2-(2-isopropyl-4,5-dimethyl-1H-imidazol-1-yl)phenoxy)pyrimidin-4-yl)-2,6-diazabicyclo[3.2.1]octane-2-carboxylate (50 mg, 0.09 mmol, 1.0 equiv) was performed according to general procedure B2. The reaction was diluted with water (2 mL), and basified to pH-8 with saturated NaHCO₃ (aq.). The resulting mixture was extracted with EtOAc (3×5 mL), the combined organic layers were washed with brine (3 mL), dried over anhydrous Na₂SO₄, and concentrated under reduced pressure to give (1S,5R)-6-(5-(4-fluoro-2-(2-isopropyl-4,5-dimethyl-1H-imidazol-1-yl)phenoxy)pyrimidin-4-yl)-2,6-diazabicyclo[3.2.1]octane (35 mg, 86%) as a colourless solid. m/z: ES⁺ [M+H]⁺=437.25.

### (1S,5R)-6-(6-(2-(2-cyclopropyl-4,5-dimethyl-1H-imidazol-1-yl)-4-fluorophenoxy)-1,2,4-triazin-5-yl)-2,6-diazabicyclo[3.2.1]octane

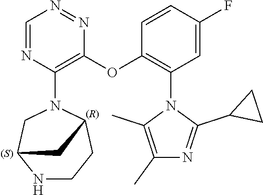

### Step 1: tert-butyl (1S,5R)-6-(6-(2-(2-cyclopropyl-4,5-dimethyl-1H-imidazol-1-yl)-4-fluorophenoxy)-1,2,4-triazin-5-yl)-2,6-diazabicyclo[3.2.1]octane-2-carboxylate

The dechlorination of tert-butyl (1S,5R)-6-(3-chloro-6-(2-(2-cyclopropyl-4,5-dimethyl-1H-imidazol-1-yl)-4-fluorophenoxy)-1,2,4-triazin-5-yl)-2,6-diazabicyclo[3.2.1]octane-2-carboxylate (155 mg, 0.27 mmol, 1.0 equiv) was performed according to general procedure D1. The resulting mixture was diluted with water (10 mL) and extracted with EtOAc (3×10 mL). The combined organic layers were dried over Na₂SO₄, and concentrated under reduced pressure. The residue was purified using method RPFC1 (0% to 100% gradient in 30 min), to afford tert-butyl (1S,5R)-6-(6-(2-(2-cyclopropyl-4,5-dimethyl-1H-imidazol-1-yl)-4-fluorophenoxy)-1,2,4-triazin-5-yl)-2,6-diazabicyclo[3.2.1]octane-2-carboxylate (110 mg, 76%) as a yellow solid. m/z: ES⁺ [M+H]⁺=536.35.

### Step 2: (1S,5R)-6-(6-(2-(2-cyclopropyl-4,5-dimethyl-1H-imidazol-1-yl)-4-fluorophenoxy)-1,2,4-triazin-5-yl)-2,6-diazabicyclo[3.2.1]octane

The Boc deprotection of tert-butyl (1S,5R)-6-(6-(2-(2-cyclopropyl-4,5-dimethyl-1H-imidazol-1-yl)-4-fluorophenoxy)-1,2,4-triazin-5-yl)-2,6-diazabicyclo[3.2.1]octane-2-carboxylate (90 mg, 0.17 mmol, 1.0 equiv) was performed according to general procedure B2. The reaction was concentrated under reduced pressure to afford (1S,5R)-6-(6-(2-(2-cyclopropyl-4,5-dimethyl-1H-imidazol-1-yl)-4-fluorophenoxy)-1,2,4-triazin-5-yl)-2,6-diazabicyclo[3.2.1]octane (HCl salt, crude). m/z: ES⁺ [M+H]⁺=436.15.

### (1S,5R)-6-(6-(4-fluoro-2-(3-isopropyl-1-methyl-6-oxo-1,6-dihydropyridin-2-yl)phenoxy)-1,2,4-triazin-5-yl)-2,6-diazabicyclo[3.2.1]octane

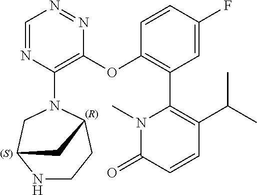

### Step 1: tert-butyl (1S,5R)-6-(6-(4-fluoro-2-(3-isopropyl-1-methyl-6-oxo-1,6-dihydropyridin-2-yl)phenoxy)-1,2,4-triazin-5-yl)-2,6-diazabicyclo[3.2.1]octane-2-carboxylate

The dechlorination of tert-butyl (1S,5R)-6-(3-chloro-6-(4-fluoro-2-(3-isopropyl-1-methyl-6-oxo-1,6-dihydropyridin-2-yl)phenoxy)-1,2,4-triazin-5-yl)-2,6-diazabicyclo[3.2.1]octane-2-carboxylate (300 mg, 0.51 mmol, 1.0 equiv) was performed according to general procedure D1. The resulting mixture was diluted with water (50 mL) and extracted with EtOAc (2×50 mL). The combined organic layers were washed with brine (2×50 mL), dried over Na₂SO₄, and concentrated under reduced pressure. The residue was purified using method RPFC1 (30% to 80% gradient in 20 min), to afford tert-butyl (1S,5R)-6-(6-(4-fluoro-2-(3-isopropyl-1-methyl-6-oxo-1,6-dihydropyridin-2-yl)phenoxy)-1,2,4-triazin-5-yl)-2,6-diazabicyclo[3.2.1]octane-2-carboxylate (270 mg, 96%) as a yellow oil. m/z: ES⁺ [M+H]⁺=551.27.

### Step 2: (1S,5R)-6-(6-(4-fluoro-2-(3-isopropyl-1-methyl-6-oxo-1,6-dihydropyridin-2-yl)phenoxy)-1,2,4-triazin-5-yl)-2,6-diazabicyclo[3.2.1]octane

The Boc deprotection of tert-butyl (1S,5R)-6-(6-(4-fluoro-2-(3-isopropyl-1-methyl-6-oxo-1,6-dihydropyridin-2-yl)phenoxy)-1,2,4-triazin-5-yl)-2,6-diazabicyclo[3.2.1]octane-2-carboxylate (220 mg, 0.40 mmol, 1.0 equiv) was performed according to general procedure B2. After completion of the reaction, the pH of the resulting mixture was adjusted to pH-8 with aq. NaOH and extracted with DCM (2×50 mL). The combined organic layers were washed with brine (2×40 mL), dried over Na₂SO₄, and concentrated under reduced pressure. This resulted in crude (1S,5R)-6-(6-(4-fluoro-2-(3-isopropyl-1-methyl-6-oxo-1,6-dihydropyridin-2-yl)phenoxy)-1,2,4-triazin-5-yl)-2,6-diazabicyclo[3.2.1]octane (260 mg, 94%). m/z: ES⁺ [M+H]⁺=451.22.

### (1S,5R)-6-(6-(4-fluoro-2-(4-isopropyl-1-methyl-1H-1,2,3-triazol-5-yl)phenoxy)-1,2,4-triazin-5-yl)-2,6-diazabicyclo[3.2.1]octane

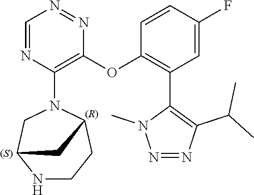

### Step 1: tert-butyl (1S,5R)-6-(6-(4-fluoro-2-(4-isopropyl-1-methyl-1H-1,2,3-triazol-5-yl)phenoxy)-1,2,4-triazin-5-yl)-2,6-diazabicyclo[3.2.1]octane-2-carboxylate

The dechlorination of tert-butyl (1S,5R)-6-(3-chloro-6-(4-fluoro-2-(4-isopropyl-1-methyl-1H-1,2,3-triazol-5-yl)phenoxy)-1,2,4-triazin-5-yl)-2,6-diazabicyclo[3.2.1]octane-2-carboxylate (200 mg, 0.36 mmol, 1.0 equiv) was performed according to general procedure D2. The resulting mixture was filtered, the solid washed with MeOH (3×10 mL) and the filtrate was concentrated under reduced pressure. The residue was purified using method RPFC1 (60% to 70% gradient in 10 min), to afford tert-butyl (1S,5R)-6-(6-(4-fluoro-2-(4-isopropyl-1-methyl-1H-1,2,3-triazol-5-yl)phenoxy)-1,2,4-triazin-5-yl)-2,6-diazabicyclo[3.2.1]octane-2-carboxylate (90 mg, 48%) as a light-yellow oil. m/z: ES⁺ [M+H]⁺=525.25.

### Step 2: (1S,5R)-6-(6-(4-fluoro-2-(4-isopropyl-1-methyl-1H-1,2,3-triazol-5-yl)phenoxy)-1,2,4-triazin-5-yl)-2,6-diazabicyclo[3.2.1]octane

The Boc deprotection of tert-butyl (1S,5R)-6-(6-(4-fluoro-2-(4-isopropyl-1-methyl-1H-1,2,3-triazol-5-yl)phenoxy)-1,2,4-triazin-5-yl)-2,6-diazabicyclo[3.2.1]octane-2-carboxylate (90 mg, 0.17 mmol, 1.0 equiv) was performed according to general procedure B2. The reaction was concentrated under reduced pressure, and EtOAc was added (10 mL). The precipitate was collected by filtration and washed with additional EtOAc (3×10 mL). This resulted in (1S,5R)-6-(6-(4-fluoro-2-(4-isopropyl-1-methyl-1H-1,2,3-triazol-5-yl)phenoxy)-1,2,4-triazin-5-yl)-2,6-diazabicyclo[3.2.1]octane (HCl salt, 60 mg, 82%) as an off-white solid. m/z: ES⁺ [M+H]⁺=425.15.

### 2-((5-((1S,5R)-2,6-diazabicyclo[3.2.1]octan-6-yl)-1,2,4-triazin-6-yl)oxy)-N-cyclopropyl-N-ethyl-5-fluorobenzamide

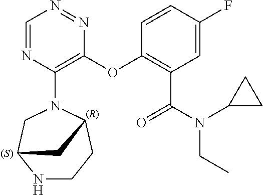

### Step 1: tert-butyl (1S,5R)-6-(6-(2-(cyclopropyl(ethyl)carbamoyl)-4-fluorophenoxy)-1,2,4-triazin-5-yl)-2,6-diazabicyclo[3.2.1]octane-2-carboxylate

The dechlorination of tert-butyl (1S,5R)-6-(3-chloro-6-(2-(cyclopropyl(ethyl)carbamoyl)-4-fluorophenoxy)-1,2,4-triazin-5-yl)-2,6-diazabicyclo[3.2.1]octane-2-carboxylate (890 mg, 1.6 mmol, 1.0 equiv) was performed according to general procedure D1. The resulting mixture was diluted with water (100 mL) and extracted with EtOAc (2×50 mL). The combined organic layers were washed with brine (2×50 mL), dried over anhydrous Na₂SO₄ and concentrated under reduced pressure. The residue was purified using method RPFC1 (30% to 80% gradient in 20 min), to afford tert-butyl (1S,5R)-6-(6-(2-(cyclopropyl(ethyl)carbamoyl)-4-fluorophenoxy)-1,2,4-triazin-5-yl)-2,6-diazabicyclo[3.2.1]octane-2-carboxylate (770 mg, 92%) as a light-yellow solid. m/z: ES⁺ [M+H]⁺=513.26.

### Step 2: 2-((5-((1S,5R)-2,6-diazabicyclo[3.2.1]octan-6-yl)-1,2,4-triazin-6-yl)oxy)-N-cyclopropyl-N-ethyl-5-fluorobenzamide

The Boc deprotection of tert-butyl (1S,5R)-6-(6-(2-(cyclopropyl(ethyl)carbamoyl)-4-fluorophenoxy)-1,2,4-triazin-5-yl)-2,6-diazabicyclo[3.2.1]octane-2-carboxylate (300 mg, 0.59 mmol, 1.0 equiv) was performed according to general procedure B2. The mixture was concentrated under reduced pressure to give 2-((5-((1S,5R)-2,6-diazabicyclo[3.2.1]octan-6-yl)-1,2,4-triazin-6-yl)oxy)-N-cyclopropyl-N-ethyl-5-fluorobenzamide (HCl salt, 230 mg, 95%) as an off-white solid. m/z: ES⁺ [M+H]⁺=413.21.

### (1S,5R)-6-(6-(4-fluoro-2-(4-isopropyl-6-methylpyrimidin-5-yl)phenoxy)-1,2,4-triazin-5-yl)-2,6-diazabicyclo[3.2.1]octane

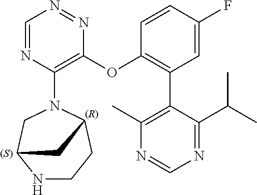

### Step 1: tert-butyl (1S,5R)-6-(6-(4-fluoro-2-(4-isopropyl-6-methylpyrimidin-5-yl)phenoxy)-1,2,4-triazin-5-yl)-2,6-diazabicyclo[3.2.1]octane-2-carboxylate

The dechlorination of tert-butyl (1S,5R)-6-(3-chloro-6-(4-fluoro-2-(4-isopropyl-6-methylpyrimidin-5-yl)phenoxy)-1,2,4-triazin-5-yl)-2,6-diazabicyclo[3.2.1]octane-2-carboxylate (400 mg, 0.70 mmol, 1.0 equiv) was performed according to general procedure D1. The reaction was quenched by the addition of water (5 mL) at 0° C., extracted with DCM (3×5 mL), dried over Na₂SO₄, and concentrated under reduced pressure. The residue was purified using method RPFC1 (10% to 100% gradient in 20 min) to afford tert-butyl (1S,5R)-6-(6-(4-fluoro-2-(4-isopropyl-6-methylpyrimidin-5-yl)phenoxy)-1,2,4-triazin-5-yl)-2,6-diazabicyclo[3.2.1]octane-2-carboxylate (300 mg, 80%) as a yellow solid. m/z: ES⁺ [M+H]⁺=536.27.

### Step 2: (1S,5R)-6-(6-(4-fluoro-2-(4-isopropyl-6-methylpyrimidin-5-yl)phenoxy)-1,2,4-triazin-5-yl)-2,6-diazabicyclo[3.2.1]octane

The Boc deprotection of tert-butyl (1S,5R)-6-(6-(4-fluoro-2-(4-isopropyl-6-methylpyrimidin-5-yl)phenoxy)-1,2,4-triazin-5-yl)-2,6-diazabicyclo[3.2.1]octane-2-carboxylate (400 mg, 0.75 mmol, 1.0 equiv) was performed according to general procedure B2. The resulting mixture was concentrated under reduced pressure and diluted with EtOAc (5 mL). The solid was collected by filtration and washed with EtOAc (3×5 mL), to afford (1S,5R)-6-(6-(4-fluoro-2-(4-isopropyl-6-methylpyrimidin-5-yl)phenoxy)-1,2,4-triazin-5-yl)-2,6-diazabicyclo[3.2.1]octane, HCl salt (320 mg, 98%) as a yellow solid. m/z: ES⁺ [M+H]⁺=436.22.

### N-(2-((5-((1S,5R)-2,6-diazabicyclo[3.2.1]octan-6-yl)-1,2,4-triazin-6-yl)oxy)-5-fluorophenyl)-N-ethyl-3-isopropyloxetan-3-amine

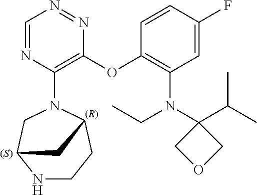

### Step 1: tert-butyl (1S,5R)-6-(6-(2-(ethyl(3-isopropyloxetan-3-yl)amino)-4-fluorophenoxy)-1,2,4-triazin-5-yl)-2,6-diazabicyclo[3.2.1]octane-2-carboxylate

The dechlorination of tert-butyl (1S,5R)-6-(3-chloro-6-(2-(ethyl(3-isopropyloxetan-3-yl)amino)-4-fluorophenoxy)-1,2,4-triazin-5-yl)-2,6-diazabicyclo[3.2.1]octane-2-carboxylate (890 mg, 1.6 mmol, 1.0 equiv) was performed according to general procedure D2. The mixture was filtered and the solids washed with MeOH (3×10 mL). The filtrate was evaporated to dryness to afford tert-butyl (1S,5R)-6-(6-(2-(ethyl(3-isopropyloxetan-3-yl)amino)-4-fluorophenoxy)-1,2,4-triazin-5-yl)-2,6-diazabicyclo[3.2.1]octane-2-carboxylate (60 mg, 71%) as a yellow oil. m/z: ES⁺ [M+H]⁺=543.30.

### Step 2: N-(2-((5-((1S,5R)-2,6-diazabicyclo[3.2.1]octan-6-yl)-1,2,4-triazin-6-yl)oxy)-5-fluorophenyl)-N-ethyl-3-isopropyloxetan-3-amine

The Boc deprotection of tert-butyl (1S,5R)-6-(6-(2-(ethyl(3-isopropyloxetan-3-yl)amino)-4-fluorophenoxy)-1,2,4-triazin-5-yl)-2,6-diazabicyclo[3.2.1]octane-2-carboxylate (50 mg, 0.09 mmol, 1.0 equiv) was performed according to general procedure B1. The mixture was concentrated under reduced pressure to give N-(2-((5-((1S,5R)-2,6-diazabicyclo[3.2.1]octan-6-yl)-1,2,4-triazin-6-yl)oxy)-5-fluorophenyl)-N-ethyl-3-isopropyloxetan-3-amine (TFA salt, 50 mg, quant.) as a colourless solid. m/z: ES⁺ [M+H]⁺=443.30.

### (1R,3R,5R)-2-(tert-butoxycarbonyl)-3-methyl-2-azabicyclo[3.1.0]hexane-3-carboxylic acid and (1R,3S,5R)-2-(tert-Butoxycarbonyl)-3-methyl-2-azabicyclo[3.1.0]hexane-3-carboxylic acid

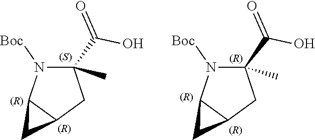

### Step 1: 2-(tert-butyl) 3-methyl (1R,3S,5R)-2-azabicyclo[3.1.0]hexane-2,3-dicarboxylate

A mixture of (1R,3S,5R)-2-(tert-butoxycarbonyl)-2-azabicyclo[3.1.0]hexane-3-carboxylic acid (600 mg, 2.6 mmol, 1.0 equiv), K₂CO₃ (365 mg, 2.6 mmol, 3.0 equiv) and MeI (375 mg, 2.6 mmol, 3.0 equiv) in DMF (5 mL) was stirred at room temperature for 1.5 h under nitrogen. The resulting mixture was filtered, then the filter cake was washed with EtOAc (3×5 mL). The resulting mixture was extracted with EtOAc (3×10 mL). The combined organic layers were washed with brine (3×10 mL) then dried over Na₂SO₄. After filtration, the filtrate was concentrated under reduced pressure. The crude product 2-(tert-butyl) 3-methyl (1R,3S,5R)-2-azabicyclo[3.1.0]hexane-2,3-dicarboxylate (500 mg, 78%) was used in the next step directly without further purification. m/z: ES⁺ [M+H−56]⁺=186.00, [M+H−100]⁺=142.05.

### Step 2: 2-(tert-butyl) 3-methyl (1R,3R,5R)-3-methyl-2-azabicyclo[3.1.0]hexane-2,3-dicarboxylate and 2-(tert-butyl) 3-methyl (1R,3S,5R)-3-methyl-2-azabicyclo[3.1.0]hexane-2,3-dicarboxylate

To a stirred solution of 2-(tert-butyl) 3-methyl (1R,3S,5R)-2-azabicyclo[3.1.0]hexane-2,3-dicarboxylate (500 mg, 2.1 mmol, 1.0 equiv) in THF (14 mL) was added LiHMDS (555 mg, 3.3 mmol, 1.6 equiv) in portions at −20° C. under nitrogen. The resulting mixture was stirred at −20° C. for 1.5 h, and MeI (471 mg, 3.3 mmol, 1.6 equiv) was then added dropwise at −20° C. The resulting mixture was stirred at room temperature for an additional 16 h. The reaction was then quenched with sat. NH₄Cl at room temperature, and extracted with EtOAc (3×25 mL). The combined organic layers were washed with brⁱne (20 mL), and dried over Na₂SO₄. After filtration, the filtrate was concentrated under reduced pressure. The crude product 2-(tert-butyl) 3-methyl (1R,3RS,5R)-3-methyl-2-azabicyclo[3.1.0]hexane-2,3-dicarboxylate (200 mg, 38%) was used in the next step directly without further purification. m/z: ES⁺ [M+H−56]⁺=200.05, [M+H−100]⁺=156.10.

### Step 3: (1R,3R,5R)-2-(tert-butoxycarbonyl)-3-methyl-2-azabicyclo[3.1.0]hexane-3-carboxylic acid and (1R,3S,5R)-2-(tert-butoxycarbonyl)-3-methyl-2-azabicyclo[3.1.0]hexane-3-carboxylic acid

A solution of 2-(tert-butyl) 3-methyl (1R,3RS,5R)-3-methyl-2-azabicyclo[3.1.0]hexane-2,3-dicarboxylate (200 mg, 0.78 mmol, 1.0 equiv) and NaOH (157 mg, 3.9 mmol, 5.0 equiv) in H₂O/MeOH (1:1, 2 mL) was stirred at 50° C. for 12 h under nitrogen. The mixture was basified to pH 6 with conc. HCl. The residue was purified using method RFPC3 (0% to 100% gradient in 15 min) to afford (1R,3RS,5R)-2-(tert-butoxycarbonyl)-3-methyl-2-azabicyclo[3.1.0]hexane-3-carboxylic acid (180 mg, 95%) as a yellow oil. m/z: ES⁺ [M+H−56]⁺=186.00, [M+H−100]⁺=142.05.

### (1S,3R,5S)-2-(tert-butoxycarbonyl)-3-methyl-2-azabicyclo[3.1.0]hexane-3-carboxylic acid and (1S,3S,5S)-2-(tert-Butoxycarbonyl)-3-methyl-2-azabicyclo[3.1.0]hexane-3-carboxylic acid

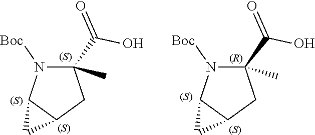

### Step 1: 2-(tert-butyl) 3-methyl (1R,3S,5R)-2-azabicyclo[3.1.0]hexane-2,3-dicarboxylate

A solution of (1S,3S,5S)-2-(tert-butoxycarbonyl)-2-azabicyclo[3.1.0]hexane-3-carboxylic acid (200 mg, 0.88 mmol, 1.0 equiv), K₂CO₃ (365 mg, 2.6 mmol, 3.0 equiv) and MeI (375 mg, 2.6 mmol, 3.0 equiv) in DMF (5 mL) was stirred at room temperature for 1.5 h under nitrogen. The resulting mixture was filtered, the filter cake was washed with EtOAc (3×5 mL). The resulting mixture was extracted with EtOAc (3×10 mL). The combined organic layers were washed with brine (3×10 mL), dried over Na₂SO₄. After filtration, the filtrate was concentrated under reduced pressure. The crude product was used in the next step directly without further purification. m/z: ES⁺ [M+H−56]⁺=186.00, [M+H−100]⁺=142.05.

### Step 2: 2-(tert-butyl) 3-methyl (1S,3R,5S)-3-methyl-2-azabicyclo[3.1.0]hexane-2,3-dicarboxylate and 2-(tert-butyl) 3-methyl (1S,3S,5S)-3-methyl-2-azabicyclo[3.1.0]hexane-2,3-dicarboxylate

To a stirred solution of 2-(tert-butyl) 3-methyl (1S,3S,5S)-2-azabicyclo[3.1.0]hexane-2,3-dicarboxylate (180 mg, 0.75 mmol, 1.0 equiv) in THF (3.6 mL) was added LiHMDS (1.0 M in hexanes, 1.2 mL, 1.2 mmol, 1.6 equiv) dropwise at −20° C. under nitrogen. The resulting mixture was stirred at −20° C. for 1.5 h, and MeI (169 mg, 1.2 mmol, 1.6 equiv) was then added dropwise at −20° C. The resulting mixture was stirred at room temperature for an additional 16 h, and then quenched with sat. NH₄Cl at room temperature, and extracted with EtOAc (3×10 mL). The combined organic layers were washed with brine (10 mL) then dried over Na₂SO₄. After filtration, the filtrate was concentrated under reduced pressure. The crude product was used in the next step directly without further purification. m/z: ES⁺ [M+H−56]⁺=200.05, [M+H−100]⁺=156.10.

### Step 3: (1S,3R,5S)-2-(tert-butoxycarbonyl)-3-methyl-2-azabicyclo[3.1.0]hexane-3-carboxylic acid and (1S,3S,5S)-2-(tert-butoxycarbonyl)-3-methyl-2-azabicyclo[3.1.0]hexane-3-carboxylic acid

A solution of 2-(tert-butyl) 3-methyl (1S,3RS,5S)-3-methyl-2-azabicyclo[3.1.0]hexane-2,3-dicarboxylate (160 mg, 0.63 mmol, 1.0 equiv) and NaOH (125 mg, 3.1 mmol, 5.0 equiv) in H₂O/MeOH (1:1, 3.2 mL) was stirred at 50° C. for 16 h under nitrogen. The mixture was acidified to pH 6 with conc. HCl. The residue was purified using method RFPC3 (0% to 100% gradient in 15 min) to afford (1S,3RS,5S)-2-(tert-butoxycarbonyl)-3-methyl-2-azabicyclo[3.1.0]hexane-3-carboxylic acid (125 mg, 83%) as a yellow oil. m/z: ES⁺ [M+H−56]⁺=186.00, [M+H−100]⁺=142.05.

### rac-5-(tert-butoxycarbonyl)-6-methyl-5-azaspiro[2.4]heptane-6-carboxylic acid

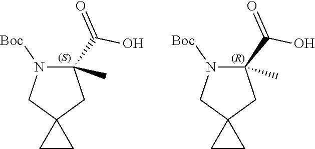

### Step 1: 5-tert-butyl 6-methyl (6S)-5-azaspiro[2.4]heptane-5,6-dicarboxylate

A solution of (6S)-5-(tert-butoxycarbonyl)-5-azaspiro[2.4]heptane-6-carboxylic acid (2.5 g, 10 mmol, 1.0 equiv), K₂CO₃ (4.5 g, 33 mmol, 3.1 equiv) and MeI (2.2 g, 16 mmol, 1.5 equiv) in THF (20 mL) was stirred at 60° C. overnight under nitrogen. The resulting mixture was diluted with water (10 mL), and extracted with EtOAc (3×20 mL). The combined organic layers were washed with brine (10 mL), and dried over Na₂SO₄. After filtration, the filtrate was concentrated under reduced pressure. The residue was purified by silica gel column chromatography (PE/EA, 1:1) to afford 5-tert-butyl 6-methyl (6S)-5-azaspiro[2.4]heptane-5,6-dicarboxylate (1.5 g, 57%) as a yellow oil. m/z: ES⁺ [M+H−56]⁺=200.05, [M+H−100]⁺=156.10.

### Step 2: rac-5-(tert-butyl) 6-methyl-5-azaspiro[2.4]heptane-5,6-dicarboxylate

A solution of 5-tert-butyl 6-methyl (6S)-5-azaspiro[2.4]heptane-5,6-dicarboxylate (1 g, 3.92 mmol, 1 equiv) in THF (30 mL) was treated with LiHMDS (1.0 M in THF) (0.9 g, 5.86 mmol, 1.50 equiv) at −20° C. for 1 h under nitrogen followed by the addition of MeI (0.83 g, 5.89 mmol, 1.5 equiv) dropwise at room temperature. The resulting mixture was stirred at room temperature for 2 h under nitrogen. The reaction was quenched with sat. NH₄Cl at 0° C. The resulting mixture was extracted with EtOAc (2×100 mL). The combined organic layers were washed with water (2×50 mL), dried over Na₂SO₄. After filtration, the filtrate was concentrated under reduced pressure to afford rac-5-(tert-butyl) 6-methyl (R)-6-methyl-5-azaspiro[2.4]heptane-5,6-dicarboxylate (1.0 g, 95%) as a brown solid. m/z: ES⁺ [M−H]⁺=254.00, [M+H−100]+=156.10.

### Step 3: rac-5-(tert-butoxycarbonyl)-6-methyl-5-azaspiro[2.4]heptane-6-carboxylic acid

The resulting mixture of rac-5-(tert-butyl) 6-methyl (R)-6-methyl-5-azaspiro[2.4]heptane-5,6-dicarboxylate (300 mg, 1.1 mmol, 1.0 equiv), NaOH (222 mg, 5.6 mmol, 5.0 equiv) in H₂O/MeOH (1:1, 4 mL) was stirred at 50° C. overnight under nitrogen. The mixture was acidified to pH 5 with 1 M HCl (aq.). The residue was purified using method RFPC4 (10% to 50% gradient in 10 min) to afford rac-(R)-5-(tert-butoxycarbonyl)-6-methyl-5-azaspiro[2.4]heptane-6-carboxylic acid (235 mg, 83%) as a brown oil. m/z: ES⁺ [M+H−56]⁺=200.05, [M+H−100]⁺=156.10.

### (1S,2R,5R)-3-(tert-butoxycarbonyl)-2-methyl-3-azabicyclo[3.1.0]hexane-2-carboxylic acid and (1S,2S,5R)-3-(tert-butoxycarbonyl)-2-methyl-3-azabicyclo[3.1.0]hexane-2-carboxylic acid

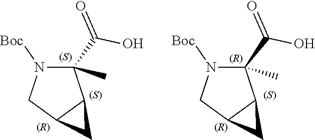

### Step 1: 3-(tert-butyl) 2-methyl (1S,2S,5R)-3-azabicyclo[3.1.0]hexane-2,3-dicarboxylate

To a stirred solution of (1S,2S,5R)-3-(tert-butoxycarbonyl)-3-azabicyclo[3.1.0]hexane-2-carboxylic acid (1 g, 4.4 mmol, 1 equiv) and MeI (1.2 g, 8.8 mmol, 2 equiv) in DMF (10 mL) was added K₂CO₃ (1.8 g, 13.2 mmol, 3 equiv) in portions at room temperature. The resulting mixture was stirred at room temperature for 5 h. The crude product was purified using method RFPC1 (10% to 100% gradient in 30 min) to afford 3-(tert-butyl) 2-methyl (1S,2S,5R)-3-azabicyclo[3.1.0]hexane-2,3-dicarboxylate (780 mg, 73%) as a colorless oil. m/z: ES⁺ [M+H]⁺=242.05.

### Step 2: 3-(tert-butyl) 2-methyl (1S,2R,5R)-2-methyl-3-azabicyclo[3.1.0]hexane-2,3-dicarboxylate and 3-(tert-butyl) 2-methyl (1S,2S,5R)-2-methyl-3-azabicyclo[3.1.0]hexane-2,3-dicarboxylate

A solution of 3-(tert-butyl) 2-methyl (1S,2S,5R)-3-azabicyclo[3.1.0]hexane-2,3-dicarboxylate (750 mg, 3.1 mmol, 1.0 equiv) in THF (8 mL) was treated with LiHMDS (1 g, 6.2 mmol, 2.0 equiv) at −78° C. for 1 h under nitrogen followed by the addition of MeI (882 mg, 6.2 mmol, 2.0 equiv) dropwise at −78° C. The resulting mixture was stirred at room temperature for 16 h under nitrogen, and the reaction was quenched with sat. NH₄Cl at room temperature. The aqueous layer was extracted with EtOAc (3×50 mL), and the combined organic layers were concentrated under vacuum to afford 3-(tert-butyl) 2-methyl (1S,2RS,5R)-2-methyl-3-azabicyclo[3.1.0]hexane-2,3-dicarboxylate (600 mg, 76%) as a colorless oil. m/z: ES⁺ [M+H]⁺=256.20.

### Step 3: (1S,2R,5R)-3-(tert-butoxycarbonyl)-2-methyl-3-azabicyclo[3.1.0]hexane-2-carboxylic acid and (1S,2S,5R)-3-(tert-butoxycarbonyl)-2-methyl-3-azabicyclo[3.1.0]hexane-2-carboxylic acid

A mixture of 3-(tert-butyl) 2-methyl (1S,2RS,5R)-2-methyl-3-azabicyclo[3.1.0]hexane-2,3-dicarboxylate (500 mg, 2.0 mmol, 1.0 equiv) and NaOH (783 mg, 20 mmol, 10 equiv) in H₂O/MeOH (1:1, 10 mL) was stirred at 50° C. for 4 h under nitrogen. The mixture was acidified to pH 7 with conc. HCl. The resulting mixture was concentrated under vacuum to afford (1S,2RS,5R)-3-(tert-butoxycarbonyl)-2-methyl-3-azabicyclo[3.1.0]hexane-2-carboxylic acid (400 mg, crude) as a colourless oil. m/z: ES⁺ [M−H]⁺=240.05.

### (2R,5S)-1-(tert-butoxycarbonyl)-2,5-dimethylpyrrolidine-2-carboxylic acid and (2S,5S)-1-(tert-butoxycarbonyl)-2,5-dimethylpyrrolidine-2-carboxylic acid

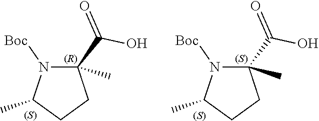

The title compound was prepared according to general procedure I1.

### Step 1: 1-(tert-butyl) 2-methyl (2S,5S)-5-methylpyrrolidine-1,2-dicarboxylate

Purified by method RFPC1 (10% to 05% gradient in 10 min) to afford 1-(tert-butyl) 2-methyl (2S,5S)-5-methylpyrrolidine-1,2-dicarboxylate (500 mg, 47%) as a colourless oil. m/z: ES⁺ [M+H−56]⁺=188.15, [M+H−100]⁺=144.20.

### Step 2: 1-(tert-butyl) 2-methyl (2R,5S)-2,5-dimethylpyrrolidine-1,2-dicarboxylate and 1-(tert-butyl) 2-methyl (2S,5S)-2,5-dimethylpyrrolidine-1,2-dicarboxylate

Purified using method RPFC1 (10% to 50% gradient in 10 min) to afford 1-tert-butyl 2-methyl (2RS,5S)-2,5-dimethylpyrrolidine-1,2-dicarboxylate (50 mg, 10%) as a colourless oil. m/z: ES⁺ [M+H]⁺=258.20.

### Step 3: (2R,5S)-1-(tert-butoxycarbonyl)-2,5-dimethylpyrrolidine-2-carboxylic acid and (2S,5S)-1-(tert-butoxycarbonyl)-2,5-dimethylpyrrolidine-2-carboxylic acid

After concentration of the reaction mixture (2RS,5S)-1-(tert-butoxycarbonyl)-2,5-dimethylpyrrolidine-2-carboxylic acid (45 mg, 95%) was obtained as alight yellow oil. m/z: ES⁺ [M+H]⁺=244.15.

### rac-2-(tert-butoxycarbonyl)-3-methyl-2-azabicyclo[2.1.1]hexane-3-carboxylic acid

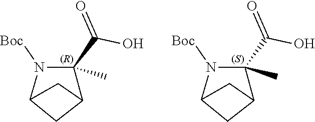

The title compound was prepared according to general procedure I1.

### Step 1: rac-2-(tert-butyl) 3-methyl-2-azabicyclo[2.1.1]hexane-2,3-dicarboxylate

Purified using method RPFC1 (10% to 100% gradient in 20 min) to afford 2-tert-butyl 3-methyl (3R)-2-azabicyclo[2.1.1]hexane-2,3-dicarboxylate (370 mg, 70%) as a light yellow oil. m/z: ES⁺ [M+H]⁺=242.30, [M+H−56]=186.05, [M+H−100]⁺=142.10.

### Step 2: rac-2-(tert-butyl) 3-methyl 3-methyl-2-azabicyclo[2.1.1]hexane-2,3-dicarboxylate

Purified using method RPFC1 (10% to 100% gradient in 20 min) to afford 2-tert-butyl 3-methyl (3R)-3-methyl-2-azabicyclo[2.1.1]hexane-2,3-dicarboxylate (100 mg, 26%) as a light yellow solid. m/z: ES⁺ [M+H]⁺=256.20.

### Step 3: rac-2-(tert-butoxycarbonyl)-3-methyl-2-azabicyclo[2.1.1]hexane-3-carboxylic acid

The crude product was used in the next step directly without further purification. m/z: ES⁺ [M+H]⁺=242.30.

### rac-(3aR,6aS)-1-benzylhexahydrocyclopenta[b]pyrrole-3a(1H)-carboxylic acid

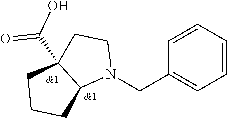 [SMILES: O=C(O)[C@@]12CCC[C@@H]1N(Cc1ccccc1)CC2]

A solution of rac-ethyl (3aR,6aS)-1-benzylhexahydrocyclopenta[b]pyrrole-3a(1H)-carboxylate (400 mg, 1.5 mmol, 1.0 equiv) and 5 N NaOH (5.0 equiv) in MeOH (5 mL) was stirred at 35° C. for 16 h. The mixture was acidified to pH 4-5 with 2 N HCl, and extracted with DCM. The combined organic layers were washed with brine, dried over Na₂SO₄, and concentrated under reduced pressure to afford rac-(3aR,6aS)-1-benzylhexa-hydrocyclopenta[b]-pyrrole-3a(1H)-carboxylic acid (100 mg, 25%) as a light yellow solid. m/z: ES⁺ [M+H]⁺=244.05.

### Compounds

### Examples 1.001 to 1.153

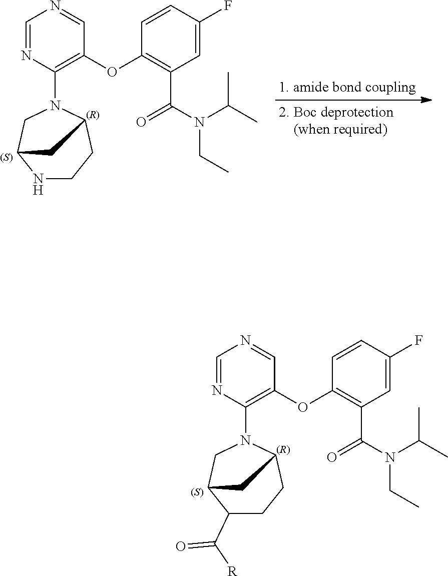

| | | Amide | | Boc | |
|---|---|---|---|---|---|
| | Carboxylic acid | coupling | Step#1 | deprotect. | Step#2 |
| Ex. | reagent | method | purification | procedure | purification |
| 1.001 | dimethylglycine | A1 | Prep-HPLC6; | — | — |
| | | | Gradient: 18% B to | | |
| | | | 38% B in 7 min; | | |
| | | | Rt = 7.58 min | | |
| 1.002 | N-(tert- | A1 | RPFC1, 10% to 100% | B2 | Prep-HPLC1; Gradient: |
| | butoxycarbonyl)-N- | | MeCN gradient in 30 | | 10% B to 40% B in 7 min; |
| | methylglycine | | min | | Rt = 6.8 min |
| 1.003 | N-(tert- | A1 | RPFC1, 10% to 50% | B2 | Prep-HPLC1; Gradient: |
| | butoxycarbonyl)-N- | | MeCN gradient in 10 | | 12% B to 38% B in 7 min; |
| | methyl-D-alanine | | min | | Rt = 7.5 min |
| 1.004 | N-(tert- | A1 | RPFC1, 10% to 50% | B2 | Prep-HPLC1; Gradient: |
| | butoxycarbonyl)-N- | | MeCN gradient in 10 | | 11% B to 36% B in 7 min; |
| | methyl-L-alanine | | min | | Rt = 7.8 min |
| 1.005 | dimethyl-D-alanine | A1 | Prep-HPLC1; | — | — |
| | hydrochloride | | Gradient: 20% B to | | |
| | | | 45% B in 7 min; | | |
| | | | Rt = 6.3 min | | |
| 1.006 | 1-[(tert- | A3 | — | B3 | Prep-HPLC12, Prep- |
| | butoxycarbonyl)ami- | | | | HPLC13 or HP-FLASH2 |
| | no]cyclopropanecarboxylic | | | | |
| | acid | | | | |
| 1.007 | 1-((tert- | A1 | RPFC1, 10% to 50% | B2 | Prep-HPLC1; Gradient: |
| | butoxycarbonyl)(meth- | | MeCN gradient in 10 | | 18% B to 41% B in 8 min; |
| | yl)amino)cyclopropane- | | min | | Rt = 8.17 min |
| | 1-carboxylic | | | | |
| | acid | | | | |
| 1.008 | (R)-1-(tert- | A1 | RPFC1, 10% to 100% | B2 | Prep-HPLC1; Gradient: |
| | butoxycarbonyl)azetidine- | | MeCN gradient in 10 | | 13% B to 36% B in 7 min; |
| | 2-carboxylic | | min | | Rt = 6.4 min |
| | acid | | | | |
| 1.009 | (S)-1-(tert- | A1 | RPFC1, 10% to 100% | B4 | Prep-HPLC1; Gradient: |
| | butoxycarbonyl)azetidine- | | MeCN gradient in 20 | | 18% B to 34% B in 7 min; |
| | 2-carboxylic | | min | | Rt = 7.5 min |
| | acid | | | | |
| 1.010 | (R)-1-(tert- | A1 | RPFC1, 10% to 100% | B4 | Prep-HPLC1; Gradient: |
| | butoxycarbonyl)-2- | | MeCN gradient in 20 | | 47% B to 54% B in 7 min; |
| | methylazetidine-2- | | min | | Rt = 6.95 min |
| | carboxylic acid | | | | |
| 1.011 | (S)-1-(tert- | A1 | RPFC1, 10% to 50% | B4 | Prep-HPLC1; Gradient: |
| | butoxycarbonyl)-2- | | MeCN gradient in 10 | | 30% B to 48% B in 7 min; |
| | methylazetidine-2- | | min | | Rt = 6.92 min |
| | carboxylic acid | | | | |
| 1.012 | (tert- | A1 | — | B2 | Prep-HPLC5; Gradient: |
| | butoxycarbonyl)-D- | | | | 3% B to 18% B in 9 min; |
| | proline | | | | Rt = 8.5 min |
| 1.013 | (tert- | A1 | RPFC1, 10% to 50% | B2 | Prep-HPLC2; Gradient: |
| | butoxycarbonyl)-L- | | MeCN gradient in 10 | | 41% B to 66% B in 10 min; |
| | proline | | min | | Rt = 9.68 min |
| 1.014 | N-methyl-D-proline | A1 | Prep-HPLC1; | — | — |
| | hydrochloride | | Gradient: 21% B to | | |
| | | | 41% B in 8 min; | | |
| | | | Rt = 7.02 min | | |
| 1.015 | (2S)-1- | A1 | RPFC1, 10% to 100% | — | — |
| | methylpyrrolidine- | | MeCN gradient in 20 | | |
| | 2-carboxylic acid | | min | | |
| 1.016 | (2R)-5- | A4 | — | — | Prep-HPLC12, Prep- |
| | oxopyrrolidine-2- | | | | HPLC13 or HP-FLASH2 |
| | carboxylic acid | | | | |
| 1.017 | (2S)-5- | A4 | — | — | Prep-HPLC12, Prep- |
| | oxopyrrolidine-2- | | | | HPLC13 or HP-FLASH2 |
| | carboxylic acid | | | | |
| 1.018 | (2S,3R)-1-(tert- | A1 | RPFC1, 10% to 60% | B1 | Prep-HPLC1; Gradient: |
| | butoxycarbonyl)-3- | | MeCN gradient in 30 | | 18% B to 41% B in 7 min; |
| | methoxy | | min | | Rt = 6.27 min |
| | pyrrolidine-2- | | | | |
| | carboxylic acid | | | | |
| 1.019 | (2S,3S)-1-(tert- | A1 | — | B2 | Prep-HPLC2; Gradient: |
| | butoxycarbonyl)-3- | | | | 42% B to 67% B in 10 min; |
| | methoxy | | | | Rt = 9.65 min |
| | pyrrolidine-2- | | | | |
| | carboxylic acid | | | | |
| 1.020 | (2S,4R)-1-(tert- | A1 | — | B2 | Prep-HPLC2; Gradient: |
| | butoxycarbonyl)-4- | | | | 35% B to 60% B in 10 min; |
| | hydroxypyrrolidine- | | | | Rt = 9.65 min |
| | 2-carboxylic acid | | | | |
| 1.021 | (25,4S)-1-(tert- | A1 | — | B2 | Prep-HPLC6; Gradient: |
| | butoxycarbonyl)-4- | | | | 18% B to 38% B in 7 min; |
| | hydroxyl | | | | Rt = 7.58 min |
| | pyrrolidine-2- | | | | |
| | carboxylic acid | | | | |
| 1.022 | (2S,3R)-1-(tert- | A1 | — | B2 | Prep-HPLC7; Gradient: |
| | butoxycarbonyl)-3- | | | | 31% B to 56% B in 10 min; |
| | hydroxypyrrolidine- | | | | Rt = 10.12 min |
| | 2-carboxylic acid | | | | |
| 1.023 | rac-(2R,3R)-1-(tert- | A1 | RPFC1, 10% to 60% | B1 | Prep-HPLC2; Gradient: |
| | butoxycarbonyl)-3- | | MeCN gradient in 30 | | 32% B to 57% B in 10 min; |
| | hydroxypyrrolidine- | | min | | Rt = 11.16 min |
| | 2-carboxylic acid | | | | |
| 1.024 | rac-(2R,4R)-1-(tert- | A1 | — | B2 | Prep-HPLC1; Gradient: |
| | butoxycarbonyl)-4- | | | | 16% B to 41% B in 7 min; |
| | fluoropyrrolidine-2- | | | | Rt = 6.1 min |
| | carboxylic acid | | | | |
| 1.025 | rac-(2R,4S)-1-(tert- | A1 | RPFC1, 10% to 60% | B1 | Prep-HPLC1; Gradient: |
| | butoxycarbonyl)-4- | | MeCN gradient in 30 | | 18% B to 41% B in 7 min; |
| | fluoropyrrolidine-2- | | min | | Rt = 6.27 min |
| | carboxylic acid | | | | |
| 1.026 | (2R,3S)-1-(tert- | A1 | RPFC1, 10% to 50% | B2 | Prep-HPLC6; Gradient: |
| | butoxycarbonyl)-3- | | MeCN gradient in 10 | | 2% B to 20% B in 7 min; |
| | fluoropyrrolidine-2- | | min | | Rt = 7.1 min |
| | carboxylic acid | | | | |
| 1.027 | (2R,3R)-1-(tert- | A1 | RPFC2, 10% to 50% | B2 | Prep-HPLC1; Gradient: |
| | butoxycarbonyl)-3- | | MeCN gradient in 10 | | 20% B to 37% B in 7 min; |
| | fluoropyrrolidine-2- | | min | | Rt = 6.65 min |
| | carboxylic acid | | | | |
| 1.028 | (1R,3S,5R)-2-(tert- | A1 | — | B2 | Prep-HPLC1; Gradient: |
| | butoxycarbonyl)-2- | | | | 20% B to 43% B in 7 min; |
| | azabicyclo[3.1.0]hexane- | | | | Rt = 6.00 min |
| | 3-carboxylic | | | | |
| | acid | | | | |
| 1.029 | (1S,3S,5S)-2-(tert- | A1 | RPFC1, 10% to 100% | B2 | Prep-HPLC8; Gradient: |
| | butoxycarbonyl)-2- | | MeCN gradient in 10 | | 18% B to 43% B in 8 min; |
| | azabicyclo[3.1.0]hexane- | | min | | Rt = 7.93 min |
| | 3-carboxylic | | | | |
| | acid | | | | |
| 1.030 | (1R,2S,5S)-3-(tert- | A1 | RPFC1, 10% to 80% | B1 | Prep-HPLC1; Gradient: |
| | butoxycarbonyl)-3- | | MeCN gradient in 20 | | 33% B to 53% B in 7 min; |
| | azabicyclo[3.1.0]hexane- | | min | | Rt = 7.05 min |
| | 2-carboxylic | | | | |
| | acid | | | | |
| 1.031 | (1S,2S,5R)-3-(tert- | A1 | — | B2 | Prep-HPLC2; Gradient: |
| | butoxycarbonyl)-3- | | | | 42% B to 67% B in 10 min; |
| | azabicyclo[3.1.0]hexane- | | | | Rt = 10.2 min |
| | 2-carboxylic | | | | |
| | acid | | | | |
| 1.032 | (S)-5-(tert- | A1 | — | B2 | Prep-HPLC1; Gradient: |
| | butoxycarbonyl)-5- | | | | 20% B to 45% B in 7 min; |
| | azaspiro[2.4]heptane- | | | | Rt = 5.50 min |
| | 6-carboxylic acid | | | | |
| 1.033 | (2S,4S)-1-(tert- | A1 | — | B2 | Prep-HPLC1; Gradient: |
| | butoxycarbonyl)-4- | | | | 24% B to 41% B in 7 min; |
| | (difluoromethyl)pyrrolidine- | | | | Rt = 6.53 min |
| | 2-carboxylic | | | | |
| | acid | | | | |
| 1.034 | (S)-1-(tert- | A1 | Prep-TLC, | B2 | Prep-HPLC1; Gradient: |
| | butoxycarbonyl)- | | DCM/MeOH 20:1 | | 25% B to 43% B in 7 min; |
| | 4,4- | | | | Rt = 6.45 min |
| | difluoropyrrolidine- | | | | |
| | 2-carboxylic acid | | | | |
| 1.035 | 1-(tert- | A3 | — | B3 | Prep-HPLC12, Prep- |
| | butoxycarbonyl)- | | | | HPLC13 or HP-FLASH2 |
| | 4,4- | | | | |
| | difluoropyrrolidine- | | | | |
| | 2-carboxylic acid | | | | |
| 1.036 | (2S,4S)-1-(tert- | A1 | RPFC1, 10% to 50% | B2 | Prep-HPLC1; Gradient: |
| | butoxycarbonyl)-4- | | MeCN gradient in 10 | | 19% B to 40% B in 7 min; |
| | methylpyrrolidine- | | min | | Rt = 7.3 min |
| | 2-carboxylic acid | | | | |
| 1.037 | (2S,4R)-1-(tert- | A1 | No purification | B2 | Prep-HPLC7; Gradient: |
| | butoxycarbonyl)-4- | | performed. RM | | 42% B to 67% B in 10 min; |
| | methylpyrrolidine- | | concentrated and used | | Rt = 9.53 min |
| | 2-carboxylic acid | | directly in next step | | |
| 1.038 | (2S,5R)-1-(tert- | A1 | RPFC1, 10% to 50% | B2 | Prep-HPLC1; Gradient: |
| | butoxycarbonyl)-5- | | MeCN gradient in 10 | | 19% B to 40% B in 7 min; |
| | methylpyrrolidine- | | min | | Rt = 7.3 min |
| | 2-carboxylic acid | | | | |
| 1.039 | (25,5S)-1-(tert- | A1 | RPFC1, 10% to 50% | B2 | No purification performed. |
| | butoxycarbonyl)-5- | | MeCN gradient in 10 | | RM concentrated to give |
| | methylpyrrolidine- | | min | | desired product |
| | 2-carboxylic acid | | | | |
| 1.040 | (2S,3S)-1-(tert- | A1 | RPFC1, 40% to 70% | B2 | Prep-HPLC1; Gradient: |
| | butoxycarbonyl)-3- | | MeCN gradient in 10 | | 16% B to 41% B in 7 min; |
| | methylpyrrolidine- | | min | | Rt = 6.10 min |
| | 2-carboxylic acid | | | | |
| 1.041 | (2S,3R)-1-(tert- | A1 | RPFC1, 10% to 100% | B2 | Prep-HPLC1; Gradient: |
| | butoxycarbonyl)-3- | | MeCN gradient in 20 | | 18% B to 38% B in 7 min; |
| | methylpyrrolidine- | | min | | Rt = 6.77 min |
| | 2-carboxylic acid | | | | |
| 1.042 | 2-(tert- | A1 | RPFC1, 10% to 100% | B2 | Prep-HPLC1; Gradient: |
| | butoxycarbonyl)-2- | | MeCN gradient in 15 | | 20% B to 37% B in 7 min; |
| | azabicyclo[2.1.1]hexane- | | min | | Rt = 6.68 min |
| | 1-carboxylic | | | | |
| | acid | | | | |
| 1.043 | (S)-1-(tert- | A1 | RPFC1, 10% to 80% | B2 | Prep-HPLC1; Gradient: |
| | butoxycarbonyl)-2- | | MeCN gradient in 10 | | 21% B to 39% B in 7 min; |
| | ethylpyrolidine-2- | | min | | Rt = 6.57 min |
| | carboxylic acid | | | | |
| 1.044 | (3R)-1-(tert- | A1 | RPFC1, 10% to 50% | B2 | Prep-HPLC1; Gradient: |
| | butoxycarbonyl)pyrrolidine- | | MeCN gradient in 10 | | 13% B to 38% B in 8 min; |
| | 3-carboxylic | | min | | Rt = 8.47 min |
| | acid | | | | |
| 1.045 | (3S)-1-(tert- | A1 | RPFC1, 60% to 70% | B1 | Prep-HPLC1; Gradient: |
| | butoxycarbonyl)pyrrolidine- | | MeCN gradient in 10 | | 11% B to 36% B in 7 min; |
| | 3-carboxylic | | min | | Rt = 6.6 min |
| | acid | | | | |
| 1.046 | (R)-1-(tert- | A1 | RPFC1, 10% to 50% | B2 | Prep-HPLC6; Gradient: |
| | butoxycarbonyl)-3- | | MeCN gradient in 10 | | 2% B to 20% B in 7 min; |
| | methylpyrrolidine- | | min | | Rt = 6.63 min |
| | 3-carboxylic acid | | | | |
| 1.047 | (S)-1-(tert- | A1 | RPFC1, 10% to 100% | B2 | Prep-HPLC6; Gradient: |
| | butoxycarbonyl)-3- | | MeCN gradient in 20 | | 3% B to 23% B in 7 min; |
| | methylpyrrolidine- | | min | | Rt = 5.95 min |
| | 3-carboxylic acid | | | | |
| 1.048 | (S)-1-(tert- | A1 | RPFC1, 10% to 50% | B2 | Prep-HPLC1; Gradient: |
| | butoxycarbonyl)piperidine- | | MeCN gradient in 10 | | 19% B to 44% B in 7 min; |
| | 2-carboxylic | | min | | Rt = 6.32 min |
| | acid | | | | |
| 1.049 | (R)-1-(tert- | A1 | RPFC1, 30% to 50% | B2 | Prep-HPLC1; Gradient: |
| | butoxycarbonyl)piperidine- | | MeCN gradient in 10 | | 18% B to 43% B in 7 min; |
| | 2-carboxylic | | min | | Rt = 6.2 min |
| | acid | | | | |
| 1.050 | rac-1-(tert- | A1 | RPFC1, 10% to 100% | Occurred | CHIRALLUX, A: |
| | butoxycarbonyl)-2- | | MeCN gradient in 20 | during | Hex(0.5% 2M NH₃—MeOH), |
| | methylpiperidine-2- | | min | step#1 | B: EtOH:MeOH = |
| | carboxylic acid | | | | 1:1; Rt = 13.1 min |
| 1.051 | rac-1-(tert- | A1 | RPFC1, 10% to 100% | Occurred | CHIRALLUX, A: |
| | butoxycarbonyl)-2- | | MeCN gradient in 20 | during | Hex(0.5% 2M |
| | methylpiperidine-2- | | min | step#1 | NH₃—MeOH), B: |
| | carboxylic acid | | | | EtOH:MeOH = 1:1; Rt = 15.6 min |
| 1.052 | (R)-1,4-bis(tert- | A3 | — | B3 | Prep-HPLC12, Prep- |
| | butoxycarbonyl)piperazine- | | | | HPLC13 or HP-FLASH2 |
| | 2-carboxylic | | | | |
| | acid | | | | |
| 1.053 | (S)-1,4-bis(tert- | A3 | — | B3 | Prep-HPLC12, Prep- |
| | butoxycarbonyl)piperazine- | | | | HPLC13 or HP-FLASH2 |
| | 2-carboxylic | | | | |
| | acid | | | | |
| 1.054 | (S)-4-(tert- | A1 | RPFC1, 10% to 50% | B2 | Prep-HPLC1; Gradient: |
| | butoxycarbonyl)morpholine-3- | | MeCN gradient in 10 | | 15% B to 38% B in 7 min; |
| | carboxylic acid | | min | | Rt = 6.6 min |
| 1.055 | (R)-4-(tert- | A1 | RPFC1, 10% to 100% | B2 | Prep-HPLC1; Gradient: |
| | butoxycarbonyl)morpholine-3- | | MeCN gradient in 20 | | 16% B to 40% B in 7 min; |
| | carboxylic acid | | min | | Rt = 6.72 min |
| 1.056 | (R)-4-(tert- | A3 | — | B3 | Prep-HPLC12, Prep- |
| | butoxycarbonyl)morpholine- | | | | HPLC13 or HP-FLASH2 |
| | 2-carboxylic acid | | | | |
| 1.057 | (S)-4-(tert- | A3 | — | B3 | Prep-HPLC12, Prep- |
| | butoxycarbonyl)morpholine-2- | | | | HPLC13 or HP-FLASH2 |
| | carboxylic acid | | | | |
| 1.058 | (S)-4-(tert- | A1 | — | B2 | Prep-HPLC1; Gradient: |
| | butoxycarbonyl)-3- | | | | 23% B to 38% B in 8 min; |
| | methylmorpholine- | | | | Rt = 7.65 min |
| | 3-carboxylic acid | | | | |
| 1.059 | (2R,3S)-4-(tert- | A1 | RPFC1, 10% to 50% | B2 | Prep-HPLC1; Gradient: |
| | butoxycarbonyl)-2- | | MeCN gradient in 10 | | 15% B to 40% B in 7 min; |
| | methylmorpholine- | | min | | Rt = 6.87 min |
| | 3-carboxylic acid | | | | |
| 1.060 | (S)-4-(tert- | A1 | RPFC1, 10% to 100% | B2 | Prep-HPLC1; Gradient: |
| | butoxycarbonyl)- | | MeCN gradient in 15 | | 20% B to 43% B in 7 min; |
| | 6,6- | | min | | Rt = 6.40 min |
| | dimethylmorpholine- | | | | |
| | 3-carboxylic acid | | | | |
| 1.061 | 2-morpholinoacetic | A1 | RPFC1, 30% to 70% | — | — |
| | acid | | MeCN gradient in 10 | | |
| | | | min | | |
| 1.062 | (R)-1-(tert- | A1 | RPFC1, 10% to 100% | B2 | Prep-HPLC1; Gradient: |
| | butoxycarbonyl)-2- | | MeCN gradient in 10 | | 17% B to 42% B in 7 min; |
| | methylpyrrolidine- | | min | | Rt = 6.0 min |
| | 2-carboxylic acid | | | | |
| 1.063 | (S)-1-(tert- | A1 | — | B2 | Prep-HPLC8; Gradient: |
| | butoxycarbonyl)-2- | | | | 17% B to 42% B in 8 min; |
| | methylpyrrolidine- | | | | Rt = 7.63 min |
| | 2-carboxylic acid | | | | |
| 1.064 | 2-(tert- | A1 | No purification | B2 | Prep-HPLC11; Gradient: |
| | butoxycarbonyl)-2- | | performed. RM | | 7% B to 22% B in 7 min; |
| | azabicyclo[3.1.0]hexane- | | concentrated and used | | Rt = 6.50 min; |
| | 1-carboxylic | | directly in next step | | CHIRALPAKIE, A: |
| | acid | | | | Hex(0.5% 2M |
| | | | | | NH₃—MeOH), B: |
| | | | | | MeCN:MeOH = 1:1; |
| | | | | | Rt = 19.79 min |
| 1.065 | 2-(tert- | A1 | No purification | B2 | Prep-HPLC11; Gradient: |
| | butoxycarbonyl)-2- | | performed. RM | | 7% B to 22% B in 7 min; |
| | azabicyclo[3.1.0]hexane- | | concentrated and used | | Rt = 6.50 min |
| | 1-carboxylic | | directly in next step | | CHIRALPAKIE, A: |
| | acid | | | | Hex(0.5% 2M |
| | | | | | NH₃—MeOH), B: |
| | | | | | MeCN:MeOH = 1:1; |
| | | | | | Rt = 25.32 min |
| 1.066 | 7-(tert- | A1 | — | B2 | Prep-HPLC1; Gradient: |
| | butoxycarbonyl)-7- | | | | 22% B to 39% B in 7 min; |
| | azabicyclo[2.2.1]heptane- | | | | Rt = 6.7 min |
| | 1-carboxylic | | | | |
| | acid | | | | |
| 1.067 | (S)-3-((tert- | A1 | RPFC1, 10% to 90% | B1 | Prep-HPLC1; Gradient: |
| | butoxycarbonyl)(meth- | | MeCN gradient in 10 | | 14% B to 36% B in 7 min; |
| | yl)amino)butanoic | | min | | Rt = 6.0 min |
| | acid | | | | |
| 1.068 | (R)-3-((tert- | A1 | RPFC1, 10% to 50% | B2 | Prep-HPLC1; Gradient: |
| | butoxycarbonyl)(meth- | | MeCN gradient in 10 | | 13% B to 38% B in 7 min; |
| | yl)amino)butanoic | | min | | Rt = 6.0 min |
| | acid | | | | |
| 1.069 | rac-3- | A1 | RPFC1, 10% to 100% | — | |
| | (dimethylamino)butanoic | | MeCN gradient in 20 | | |
| | acid | | min; | | |
| | | | CHIRALPAKIG, | | |
| | | | isocratic Hex(0.5% 2M | | |
| | | | NH₃—MeOH): | | |
| | | | (EtOH:DCM = 1:1) = | | |
| | | | 60:40; Rt = 8.75 | | |
| | | | min | | |
| 1.070 | rac-3- | A1 | RPFC1, 10% to 100% | — | |
| | (dimethylamino)butanoic | | MeCN gradient in 20 | | |
| | acid | | min; | | |
| | | | CHIRALPAKIG, | | |
| | | | isocratic Hex(0.5% | | |
| | | | 2M NH₃—MeOH): | | |
| | | | (EtOH:DCM = 1:1) = | | |
| | | | 60:40; Rt = 13.79 | | |
| | | | min | | |
| 1.071 | (S)-2-(1-(tert- | A3 | — | B3 | Prep-HPLC12, Prep- |
| | butoxycarbonyl)pyrrolidin- | | | | HPLC13 or HP-FLASH2 |
| | 3-yl)acetic | | | | |
| | acid | | | | |
| 1.072 | (R)-2-(1- | A1 | Prep-HPLC1; | — | |
| | methylpyrrolidin-3- | | Gradient: 15% B to | | |
| | yl)acetic acid | | 40% B in 7 min; | | |
| | | | Rt = 6.27 min | | |
| 1.073 | (R)-2-(1-(tert- | A3 | — | B3 | Prep-HPLC12, Prep- |
| | butoxycarbonyl)pyrrolidin- | | | | HPLC13 or HP-FLASH2 |
| | 2-yl)acetic | | | | |
| | acid | | | | |
| 1.074 | (RS)-2-((R)-1-(tert- | A1 | RPFC1, 10% to 50% | B2 | Prep-HPLC1; Gradient: |
| | butoxycarbonyl)pyrrolidin- | | MeCN gradient in 10 | | 17% B to 42% B in 8 min; |
| | 2-yl)propanoic acid | | min | | Rt = 6.95 min; |
| | | | | | CHIRALPAKIM, isocratic |
| | | | | | A: Hex(0.2% IPA), B: |
| | | | | | EtOH:DCM = 1:1; |
| | | | | | Rt = 9.52 min |
| 1.075 | (RS)-2-((R)-1-(tert- | A1 | RPFC1, 10% to 50% | B2 | Prep-HPLC1; Gradient: |
| | butoxycarbonyl)pyrrolidin-2- | | MeCN gradient in 10 | | 17% B to 42% B in 8 min; |
| | yl)propanoic acid | | min | | CHIRALPAKIM, isocratic |
| | | | | | A: Hex(0.2% IPA), B: |
| | | | | | EtOH:DCM = 1:1; |
| | | | | | Rt = 13.81 min |
| 1.076 | (RS)-2-((R)-1-(tert- | A1 | RPFC2, 0% to 100% | B2 | CHIRALPAKIK, isocratic |
| | butoxycarbonyl)pyrrolidin-3- | | MeCN gradient in 15 | | Hex(0.5% 2M NH₃—MeOH): |
| | yl)propanoic acid | | min | | (EtOH:DCM = 1:1) = |
| | | | | | 70:30; Rt = 12.9 min |
| 1.077 | (RS)-2-((R)-1-(tert- | A1 | RPFC2, 0% to 100% | B2 | CHIRALPAKIK, isocratic |
| | butoxycarbonyl)pyrrolidin-3- | | MeCN gradient in 15 | | Hex(0.5% 2M NH₃—MeOH): |
| | yl)propanoic acid | | min | | (EtOH:DCM = 1:1) = |
| | | | | | 70:30; Rt = 16.05 min |
| 1.078 | rac-2-(1-(tert- | A1 | RPFC1, 10% to 50% | B2 | Prep-HPLC2; Gradient: |
| | butoxycarbonyl)-3- | | MeCN gradient in 10 | | 2% B to 13% B in 10 min; |
| | methylpyrrolidin-3- | | min | | Rt = 9.87 min |
| | yl)acetic acid | | | | |
| 1.079 | Rac-(3aS,6aS)-2- | A1 | — | B2 | Prep-HPLC1; Gradient: |
| | (tert- | | | | 20% B to 40% B in 7 min; |
| | butoxycarbonyl)hexa- | | | | Rt = 6.43 min |
| | hydrocyclopenta[c] | | | | CHIRALPAKIM, isocratic |
| | pyrrole-3a(1H)- | | | | A: Hex(0.2% IPAmine), B: |
| | carboxylic acid | | | | EtOH:DCM = 1:1; Rt = 5.31 |
| | | | | | min |
| 1.080 | Rac-(3aS,6aS)-2- | A1 | — | B2 | Prep-HPLC1; Gradient: |
| | (tert- | | | | 20% B to 40% B in 7 min; |
| | butoxycarbonyl)hexa- | | | | Rt = 6.43 min |
| | hydrocyclopenta[c] | | | | CHIRALPAKIM, isocratic |
| | pyrrole-3a(1H)- | | | | A: Hex(0.2% IPAmine), B: |
| | carboxylic acid | | | | EtOH:DCM = 1:1; Rt = 7.45 |
| | | | | | min |
| 1.081 | trans-3-((tert- | A3 | — | B3 | Prep-HPLC12, Prep- |
| | butoxycarbonyl)ami- | | | | HPLC13 or HP-FLASH2 |
| | no)cyclobutanecarboxylic | | | | |
| | acid | | | | |
| 1.082 | cis-3-(tert- | A3 | — | B3 | Prep-HPLC12, Prep- |
| | butoxycarbonylami- | | | | HPLC13 or HP-FLASH2 |
| | no)cyclobutanecarboxylic | | | | |
| | acid | | | | |
| 1.083 | 5-((tert- | A3 | — | B3 | Prep-HPLC12, Prep- |
| | butoxycarbonyl)ami- | | | | HPLC13 or HP-FLASH2 |
| | no)pentanoic acid | | | | |
| 1.084 | 2-(1-(tert- | A3 | — | B3 | Prep-HPLC12, Prep- |
| | butoxycarbonyl)azetidin- | | | | HPLC13 or HP-FLASH2 |
| | 3-yl)acetic acid | | | | |
| 1.085 | rac-2-(1-(tert- | A1 | RPFC1, 30% to 80% | B4 | RPFC5; 30% B to 80% |
| | butoxycarbonyl)azetidin- | | MeCN gradient in 25 | | MeCN gradient in 35 min; |
| | 3-yl)propanoic | | min | | CHIRALPAKIE, isocratic |
| | acid | | | | A: Hex(0.2% IPAmine), B: |
| | | | | | EtOH:DCM = 1:1; |
| | | | | | Rt = 12.11 min |
| 1.086 | rac-2-(1-(tert- | A1 | RPFC1, 30% to 80% | B4 | RPFC5; 30% B to 80% |
| | butoxycarbonyl)azetidin- | | MeCN gradient in 25 | | MeCN gradient in 35 min; |
| | 3-yl)propanoic | | min | | CHIRALPAKIE, isocratic |
| | acid | | | | A: Hex(0.2% IPAmine), B: |
| | | | | | EtOH:DCM = 1:1; |
| | | | | | Rt = 14.98 min |
| 1.087 | 1-(tert- | A3 | — | B3 | Prep-HPLC12, Prep- |
| | butoxycarbonyl)-3- | | | | HPLC13 or HP-FLASH2 |
| | fluoroazetidine-3- | | | | |
| | carboxylic acid | | | | |
| 1.088 | 2-(N- | A3 | — | — | Prep-HPLC12, Prep- |
| | methylacetamido)acetic | | | | HPLC13 or HP-FLASH2 |
| | acid | | | | |
| 1.089 | (R)-1-(tert- | A3 | — | B3 | Prep-HPLC12, Prep- |
| | butoxycarbonyl)piperidine- | | | | HPLC13 or HP-FLASH2 |
| | 3-carboxylic | | | | |
| | acic | | | | |
| 1.090 | (S)-1-(tert- | A3 | — | B3 | Prep-HPLC12, Prep- |
| | butoxycarbonyl)piperidine- | | | | HPLC13 or HP-FLASH2 |
| | 3-carboxylic | | | | |
| | acid | | | | |
| 1.091 | 1- | A3 | — | B3 | Prep-HPLC12, Prep- |
| | (ethoxycarbonyl)piperidine- | | | | HPLC13 or HP-FLASH2 |
| | 4-carboxylic | | | | |
| | acid | | | | |
| 1.092 | (R)-3-((tert- | A3 | — | B3 | Prep-HPLC12, Prep- |
| | butoxycarbonyl)ami- | | | | HPLC13 or HP-FLASH2 |
| | no)butanoic acid | | | | |
| 1.093 | 2-(4-(tert- | A3 | — | B3 | Prep-HPLC12, Prep- |
| | butoxycarbonyl)piperazin- | | | | HPLC13 or HP-FLASH2 |
| | 1-yl)acetic | | | | |
| | acid | | | | |
| 1.094 | (4-methylpiperazin- | A3 | — | — | Prep-HPLC12, Prep- |
| | 1-yl)acetic acid | | | | HPLC13 or HP-FLASH2 |
| 1.095 | (S)-3-((tert- | A3 | — | B3 | Prep-HPLC12, Prep- |
| | butoxycarbonyl)amino)-2- | | | | HPLC13 or HP-FLASH2 |
| | methylpropanoic | | | | |
| | acid | | | | |
| 1.096 | 3-methoxypropanoic | A3 | — | — | Prep-HPLC12, Prep- |
| | acid | | | | HPLC13 or HP-FLASH2 |
| 1.097 | (S)-2-((tert- | A3 | — | B3 | Prep-HPLC12, Prep- |
| | butoxycarbonyl)amino)-3- | | | | HPLC13 or HP-FLASH2 |
| | methoxypropanoic | | | | |
| | acid | | | | |
| 1.098 | (S)-4-((tert- | A3 | — | B3 | Prep-HPLC12, Prep- |
| | butoxycarbonyl)ami- | | | | HPLC13 or HP-FLASH2 |
| | no)pentanoic acid | | | | |
| 1.099 | 3-[(tert- | A3 | — | B3 | Prep-HPLC12, Prep- |
| | butoxycarbonyl)(meth- | | | | HPLC13 or HP-FLASH2 |
| | yl)amino]propanoic | | | | |
| | acid | | | | |
| 1.100 | (S)-3-((tert- | A3 | — | B3 | Prep-HPLC12, Prep- |
| | butoxycarbonyl)ami- | | | | HPLC13 or HP-FLASH2 |
| | no)butanoic acid | | | | |
| 1.101 | (R)-2-((tert- | A3 | — | B3 | Prep-HPLC12, Prep- |
| | butoxycarbonyl)amino)-3- | | | | HPLC13 or HP-FLASH2 |
| | methoxypropanoic | | | | |
| | acid | | | | |
| 1.102 | 1-(tert- | A3 | — | B3 | Prep-HPLC12, Prep- |
| | butoxycarbonyl)azetidine- | | | | HPLC13 or HP-FLASH2 |
| | 3-carboxylic | | | | |
| | acid | | | | |
| 1.103 | (1R,3RS,5R)-2-(tert- | A1 | RPFC3, 0% to 100% | B2 | RPFC1, 0% to 100% |
| | butoxycarbonyl)-3- | | gradient in 15 min | | MeCN gradient in 15 min |
| | methyl-2- | | | | |
| | azabicyclo[3.1.0]hexane- | | | | |
| | 3-carboxylic | | | | |
| | acid | | | | |
| 1.104 | (1S,3RS,5S)-2-(tert- | A1 | RPFC3, 0% to 100% | B2 | RPFC1, 0% to 100% |
| | butoxycarbonyl)-3- | | gradient in 15 min | | MeCN gradient in 15 min |
| | methyl-2- | | | | |
| | azabicyclo[3.1.0]hexane- | | | | |
| | 3-carboxylic | | | | |
| | acid | | | | |
| 1.105 | rac-2-(tert- | A1 | RPFC1, 10% to 50% | B2 | Prep-HPLC1; Gradient: |
| | butoxycarbonyl)-2- | | MeCN gradient in 10 | | 16% B to 41% B in 7 min; |
| | azabicyclo[2.1.1]hexane- | | min | | Rt = 3.88 min; |
| | 3-carboxylic | | | | CHIRALPAKIG, isocratic |
| | acid | | | | A: Hex(0.2% DEA): |
| | | | | | (EtOH:DCM = 1:1) = 50:50; |
| | | | | | Rt = 5.12 min |
| 1.106 | rac-2-(tert- | A1 | RPFC1, 10% to 50% | B2 | Prep-HPLC1; Gradient: |
| | butoxycarbonyl)-2- | | MeCN gradient in 10 | | 16% B to 41% B in 7 min; |
| | azabicyclo[2.1.1]hexane- | | min | | Rt = 3.88 min; |
| | 3-carboxylic | | | | CHIRALPAKIG, isocratic |
| | acid | | | | A: Hex(0.2% DEA): |
| | | | | | (EtOH:DCM = 1:1) = 50:50; |
| | | | | | Rt = 12.1 min |
| 1.107 | tetrahydro-1H- | A1 | Prep-HPLC15; | — | — |
| | pyrrolizine-7a(5H)- | | Gradient: 7% B to | | |
| | carboxylic acid | | 30% B in 7 min; | | |
| | | | Rt = 6.30 min | | |
| 1.108 | (R)-1-(tert- | A1 | RPFC1, 10% to 50% | | Prep-HPLC16; Gradient: |
| | butoxycarbonyl)azepane- | | MeCN gradient in 10 | | 2% B to 24% B in 7 min; |
| | 2-carboxylic | | min | | Rt = 6.73 min |
| | acid | | | | |
| 1.109 | (2S)-1-(tert- | A1 | RPFC1, 40% to 70% | B2 | Prep-HPLC1; Gradient: |
| | butoxycarbonyl)azepane- | | MeCN gradient in 10 | | 9% B to 27% B in 7 min; |
| | 2-carboxylic | | min | | Rt = 7.43 min |
| | acid | | | | |
| 1.110 | rac-(R)-5-(tert- | A1 | RPFC4, 10% to 50% | B2 | Prep-HPLC1; Gradient: |
| | butoxycarbonyl)-6- | | MeCN gradient in 10 | | 10% B to 35% B in 8 min; |
| | methyl-5- | | min | | Rt = 7.93 min |
| | azaspiro[2.4]heptane- | | | | then |
| | 6-carboxylic acid | | | | CHIRALPAKIM, A: |
| | | | | | Hex(0.2% IPAmine), B: |
| | | | | | EtOH:DCM = 1:1; gradient |
| | | | | | 5% B to 50% B in 7 min; |
| | | | | | Rt = 5.73 min |
| 1.111 | rac-(R)-5-(tert- | A1 | RPFC4, 10% to 50% | B2 | Prep-HPLC1; Gradient: |
| | butoxycarbonyl)-6- | | MeCN gradient in 10 | | 10% B to 35% B in 8 min; |
| | methyl-5- | | min | | Rt = 7.93 min |
| | azaspiro[2.4]heptane- | | | | then |
| | 6-carboxylic acid | | | | CHIRALPAKIM, A: |
| | | | | | Hex(0.2% IPAmine), B: |
| | | | | | EtOH:DCM = 1:1; gradient |
| | | | | | 5% B to 50% B in 7 min; |
| | | | | | Rt = 4.26 min |
| 1.112 | (1R,2RS,5S)-3-(tert- | — | — | B1 | Prep-HPLC1; Gradient: |
| | butoxycarbonyl)-2- | | | | 25% B to 48% B in 7 min; |
| | methyl-3- | | | | Rt = 6.13 min |
| | azabicyclo[3.1.0]hexane- | | | | |
| | 2-carboxylic | | | | |
| | acid | | | | |
| 1.113 | (1S,2RS,5R)-3-(tert- | A1 | RPFC1, 10% to 100% | B2 | RPFC1, 10% to 100% |
| | butoxycarbonyl)-2- | | MeCN gradient in 10 | | MeCN gradient in 10 min |
| | methyl-3- | | min | | |
| | azabicyclo[3.1.0]hexane- | | | | |
| | 2-carboxylic | | | | |
| | acid | | | | |
| 1.114 | 2-(1H-imidazol-1- | A1 | Prep-HPLC9; | — | — |
| | yl)acetic acid | | Gradient: 7% B to | | |
| | | | 31% B in 7 min; | | |
| | | | Rt = 6.5 min | | |
| 1.115 | 2-(4-methyl-1H- | A1 | CHIRALPAKIM, | — | — |
| | imidazol-1-yl)acetic | | isocratic Hex(0.5% 2M | | |
| | acid | | NH₃—MeOH): | | |
| | | | (EtOH:DCM = 1:1) = | | |
| | | | 70:30; Rt = 22.35 | | |
| | | | min | | |
| 1.116 | 2-(2-methyl-1H- | A1 | Prep-HPLC10; | — | — |
| | imidazol-1-yl)acetic | | Gradient: 10% B to | | |
| | acid | | 30% B in 8 min; | | |
| | | | Rt = 7.10 min | | |
| 1.117 | (R)-2-(1H-imidazol- | A1 | Prep-HPLC11; | — | — |
| | 1-yl)propanoic acid | | Gradient: 6% B to | | |
| | | | 23% B in 8 min; | | |
| | | | Rt = 7.18 min | | |
| 1.118 | rac-2-(1H-imidazol- | A1 | RPFC1, 10% to 100% | — | — |
| | 1-yl)propanoic acid | | MeCN gradient in 20 | | |
| | | | min; | | |
| | | | CHIRALPAKIK, | | |
| | | | isocratic A: | | |
| | | | Hex(0.5% 2M | | |
| | | | NH₃—MeOH), B: | | |
| | | | EtOH:DCM = 1:1; 60:40 | | |
| | | | Rt = 8.42 min | | |
| 1.119 | 2-(5-methyl-1H- | A1 | CHIRALPAKIM, | | |
| | imidazol-1-yl)acetic | | isocratic Hex(0.5% | | |
| | acid | | 2M NH₃—MeOH): | | |
| | | | (EtOH:DCM = 1:1) = | | |
| | | | 70:30; Rt = 28.51 | | |
| | | | min | | |
| 1.120 | 2-(1-methyl-1H- | A1 | Prep-HPLC6; | — | — |
| | imidazol-4-yl)acetic | | Gradient: 2% B to | | |
| | acid | | 15% B in 7 min; | | |
| | | | Rt = 6.93 min | | |
| 1.121 | 2-(1-methyl-1H- | A1 | Prep-HPLC1; | — | — |
| | imidazol-2-yl)acetic | | Gradient: 20% B to | | |
| | acid | | 40% B in 8 min; | | |
| | | | Rt = 6.93 min | | |
| 1.122 | 2-(1-methyl-1H- | A2 | Prep-HPLC1; | — | — |
| | imidazol-5-yl)acetic | | Gradient: 25% B to | | |
| | acid | | 50% B in 7 min; | | |
| | | | Rt = 5.82 min | | |
| 1.123 | 1,2,4-triazole-1- | A5 | — | — | Prep-HPLC12, Prep- |
| | acetic acid | | | | HPLC13 or HP-FLASH2 |
| 1.124 | 1-methyl-1H-1,2,3- | A5 | — | — | Prep-HPLC12, Prep- |
| | triazole-5- | | | | HPLC13 or HP-FLASH2 |
| | carboxylic acid | | | | |
| 1.125 | 1-methyl-1H-1,2,4- | A5 | — | — | Prep-HPLC12, Prep- |
| | triazole-5- | | | | HPLC13 or HP-FLASH2 |
| | carboxylic acid | | | | |
| 1.126 | pyrimidine-4- | A5 | — | — | Prep-HPLC12, Prep- |
| | carboxylic acid | | | | HPLC13 or HP-FLASH2 |
| 1.127 | 5-fluoropyridine-3- | A5 | — | — | Prep-HPLC12, Prep- |
| | carboxylic acid | | | | HPLC13 or HP-FLASH2 |
| 1.128 | pyridazine-4- | A5 | — | — | Prep-HPLC12, Prep- |
| | carboxylic acid | | | | HPLC13 or HP-FLASH2 |
| 1.129 | pyrimidine-2- | A5 | — | — | Prep-HPLC12, Prep- |
| | carboxylic acid | | | | HPLC13 or HP-FLASH2 |
| 1.130 | pyrimidine-5- | A5 | — | — | Prep-HPLC12, Prep- |
| | carboxylic acid | | | | HPLC13 or HP-FLASH2 |
| 1.131 | pyridazine-3- | A5 | — | — | Prep-HPLC12, Prep- |
| | carboxylic acid | | | | HPLC13 or HP-FLASH2 |
| 1.132 | 2-pyrazinecarboxylic | A5 | — | — | Prep-HPLC12, Prep- |
| | acid | | | | HPLC13 or HP-FLASH2 |
| 1.133 | 1H-1,2,4-triazole-5- | A4 | — | — | Prep-HPLC12, Prep- |
| | carboxylic acid | | | | HPLC13 or HP-FLASH2 |
| 1.134 | 1-methyl-1H- | A5 | — | — | Prep-HPLC12, Prep- |
| | imidazole-2- | | | | HPLC13 or HP-FLASH2 |
| | carboxylic acid | | | | |
| 1.135 | 1H-imidazole-2- | A4 | — | — | Prep-HPLC12, Prep- |
| | carboxylic acid | | | | HPLC13 or HP-FLASH2 |
| 1.136 | oxazole-4- | A5 | — | — | Prep-HPLC12, Prep- |
| | carboxylic acid | | | | HPLC13 or HP-FLASH2 |
| 1.137 | 2H-pyrazole-3- | A4 | — | — | Prep-HPLC12, Prep- |
| | carboxylic acid | | | | HPLC13 or HP-FLASH2 |
| 1.138 | 1H-imidazole-5- | A4 | — | — | Prep-HPLC12, Prep- |
| | carboxylic acid | | | | HPLC13 or HP-FLASH2 |
| 1.139 | 2-methylpyrazole-3- | A5 | — | — | Prep-HPLC12, Prep- |
| | carboxylic acid | | | | HPLC13 or HP-FLASH2 |
| 1.140 | 2-hydroxy-2- | A1 | Prep-HPLC1; | — | — |
| | methylpropanoic | | Gradient: 22% B to | | |
| | acid | | 39% B in 7 min; | | |
| | | | Rt = 6.67 min | | |
| 1.141 | (S)-2- | A1 | Prep-HPLC1; | — | — |
| | hydroxypropanoic | | Gradient: 2% B to | | |
| | acid | | 32% B in 8 min; | | |
| | | | Rt = 7.48 min | | |
| 1.142 | (R)-2- | A1 | Prep-HPLC1; | — | — |
| | hydroxypropanoic | | Gradient: 19% B to | | |
| | acid | | 36% B in 7 min; | | |
| | | | Rt = 6.8 min | | |
| 1.143 | 2- | A5 | — | — | Prep-HPLC12, Prep- |
| | (trifluoromethoxy)acetic | | | | HPLC13 or HP-FLASH2 |
| | acid | | | | |
| 1.144 | (R)-2-(1-(tert- | A1 | RPFC1, 40% to 80% | B2 | Prep-HPLC1; Gradient: |
| | butoxycarbonyl)piperidin- | | MeCN gradient in 10 | | 14% B to 39% B in 8 min; |
| | 3-yl)acetic acid | | min | | Rt = 6.63 min |
| 1.145 | (S)-2-(1-(tert- | A1 | RPFC1, 20% to 70% | B2 | Prep-HPLC1; Gradient: |
| | butoxycarbonyl)piperidin- | | MeCN gradient in 10 | | 18% B to 37% B in 7 min; |
| | 3-yl)acetic acid | | min | | Rt = 6.15 min |
| 1.146 | 3-((tert- | A1 | RPFC1, 30% to 60% | B2 | 1. Prep-HPLC1; Gradient: |
| | butoxycarbonyl)amino)-2- | | MeCN gradient in 10 | | 30% B to 60% B in 8 min; |
| | methylbutanoic acid | | min | | Rt = 7.15 min; |
| | | | | | 2. Prep-SFC1; A: CO₂; B: |
| | | | | | MeOH (1% 2 M NH₃ in |
| | | | | | MeOH); Isocratic: 17% B; |
| | | | | | Rt = 6.60 min. |
| | | | | | 3. CHIRALPAKIM, A: |
| | | | | | Hex (0.2% IPAmine), B: |
| | | | | | EtOH:DCM = 1:1; Isocratic |
| | | | | | (65:35); Rt = 8.18 min |
| 1.147 | 3-((tert- | A1 | RPFC1, 30% to 60% | B2 | 1. Prep-HPLC1; Gradient: |
| | butoxycarbonyl)amino)-2- | | MeCN gradient in 10 | | 30% B to 60% B in 8 min; |
| | methylbutanoic acid | | min | | Rt = 7.15 min; |
| | | | | | 2. Prep-SFC1; A: CO₂; B: |
| | | | | | MeOH (1% 2M NH₃ in |
| | | | | | MeOH); Isocratic: 17% B; |
| | | | | | Rt = 6.60 min. |
| | | | | | 3. CHIRALPAKIM, A: |
| | | | | | Hex (0.2% IPAmine), B: |
| | | | | | EtOH:DCM = 1:1; Isocratic |
| | | | | | (65:35); Rt = 10.36 min |
| 1.148 | 1-tert-butyl 2- | A1 | RPFC1, 10% to 100% | B2 | Prep-HPLC1; Gradient: |
| | methyl (2RS,5S)- | | MeCN gradient in 15 | | 21% B to 46% B in 7 min; |
| | 2,5- | | min | | Rt = 6.12 min |
| | dimethylpyrrolidine- | | | | |
| | 1,2-dicarboxylate | | | | |
| 1.149 | rac-2-(tert- | A1 | RPFC1, 10% to 100% | B2 | Prep-HPLC1; Gradient: |
| | butoxycarbonyl)-3- | | MeCN gradient in 20 | | 17% B to 42% B in 7 min; |
| | methyl-2- | | min | | Rt = 6.32 min |
| | azabicyclo[2.1.1]hexane- | | | | |
| | 3-carboxylic | | | | |
| | acid | | | | |
| 1.150 | 1- | A1 | RPFC7, 42% to | — | — |
| | azabicyclo[3.3.1]nonane- | | 67% MeOH gradient | | |
| | 5-carboxylic | | in 10 min | | |
| | acid | | | | |
| 1.151 | (3aR,6aR)-2- | A1 | Prep-HPLC16; | — | — |
| | methylhexahydro- | | Gradient: 5% B to | | |
| | cyclopenta[c]pyrrole- | | 17% B in 5 min; Rt = | | |
| | 3a(1H)-carboxylic | | 5.1 min | | |
| | acid | | | | |
| 1.152 | rac-(3aR,6aR)-5- | A1 | Prep-HPLC1; | — | — |
| | benzyltetrahydro- | | Gradient: 40% B to | | |
| | 1H-furo[3,4- | | 55% B in 7 min; Rt = | | |
| | c]pyrrole-3a(3H)- | | 7.2 min | | |
| | carboxylic acid | | | | |
| 1.153 | rac-(R)-1-(tert- | A1 | NPFC1; A: DCM, B = | B2 | NPFC1; A: DCM + 1% |
| | butoxycarbonyl)-2- | | MeOH; Gradient: 0% | | Et₃N, B = MeOH; |
| | (methoxymethyl)pyrrolidine-2- | | B to 2% B. | | Gradient: 0% B to 5% B |
| | carboxylic acid | | | | |
| 1.154 | rac-(1R,5R)-3-(tert- | A1 | RPFC1, 40% to 100% | B2 | CHIRALPAK IK, |
| | butoxycarbonyl)-3- | | MeCN gradient in 10 | | Hex(0.2% IPAmine): |
| | azabicyclo[3.2.0]heptane- | | min | | (IPA:DCM = 1:1) = 35% to 55% |
| | 1-carboxylic | | | | B; Rt = 9.55 min |
| | acid | | | | |
| 1.155 | rac-(1R,5R)-3-(tert- | A1 | RPFC1, 40% to 100% | B2 | CHIRALPAK IK, |
| | butoxycarbonyl)-3- | | MeCN gradient in 10 | | Hex(0.2% IPAmine): |
| | azabicyclo[3.2.0]heptane- | | min | | (IPA:DCM = 1:1) = 35% to 55% |
| | 1-carboxylic | | | | B; Rt = 12.93 min |
| | acid | | | | |

### Example 1.001: 2-((4-((1S,5R)-2-(dimethylglycyl)-2,6-diazabicyclo[3.2.1]octan-6-yl)pyrimidin-5-yl)oxy)-N-ethyl-5-fluoro-N-isopropylbenzamide

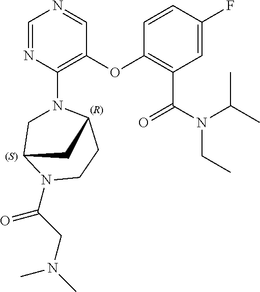

Off-white solid (34 mg, 37%).

¹H NMR (400 MHz, DMSO-d₆, 295 K) δ 8.43-8.23 (m, 1H), 8.22-8.13 (m, 1H), 7.97-7.52 (m, 1H), 7.47-7.18 (m, 2H), 7.15-6.86 (m, 1H), 5.29-5.16 (m, 1H), 4.73-4.33 (m, 1H), 4.32-4.01 (m, 1H), 3.68-3.32 (m, 5H), 2.95-2.75 (m, 2H), 2.32-2.01 (m, 6H), 2.00-1.85 (m, 2H), 1.84-1.54 (m, 2H), 1.42-0.42 (m, 9H). m/z: ES⁺ [M+H]⁺=499.25.

### Example 1.002: N-ethyl-5-fluoro-N-isopropyl-2-((4-((1S,5R)-2-(methylglycyl)-2,6-diazabicyclo[3.2.1]octan-6-yl)pyrimidin-5-yl)oxy)benzamide

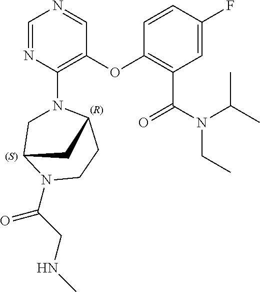

Colourless solid (44 mg, 31% over 2 steps).

¹H NMR (400 MHz, DMSO-d₆, 297 K) δ 8.40-8.30 (m, 1H), 7.89-7.71 (m, 1H), 7.30-7.20 (m, 2H), 7.12-6.88 (m, 1H), 5.23-4.79 (m, 1H), 4.61 (s, 1H), 4.47-4.01 (m, 1H), 3.85-3.75 (m, 2H), 3.70-3.47 (m, 1H), 3.40-3.30 (m, 1H), 3.28-3.21 (m, 1H), 3.20-3.16 (m, 1H), 3.16-3.08 (m, 1H), 3.07-2.95 (m, 1H), 2.82-2.60 (m, 1H), 2.32-2.22 (m, 3H), 1.97-1.81 (m, 2H), 1.79-1.48 (m, 2H), 1.28-1.08 (m, 5H), 1.08-0.93 (m, 4H). m/z: ES⁺ [M+H]⁺=485.25.

### Example 1.003: N-ethyl-5-fluoro-N-isopropyl-2-((4-((1S,5R)-2-(methyl-D-alanyl)-2,6-diazabicyclo[3.2.1]octan-6-yl)pyrimidin-5-yl)oxy)benzamide

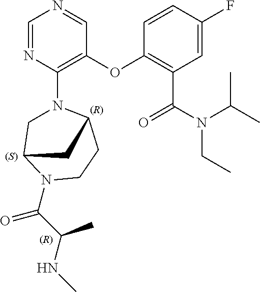

Colourless solid (25 mg, 24% over 2 steps).

¹H NMR (400 MHz, DMSO-d₆, 295 K) δ 8.43-8.31 (m, 1H), 7.89-7.76 (m, 1H), 7.49-7.23 (m, 2H), 7.13-6.98 (m, 1H), 5.21-5.11 (m, 1H), 4.92.4.78 (m, 1H), 4.77-4.26 (m, 1H), 4.24-3.74 (m, 3H), 3.74-3.49 (m, 1H), 3.54-3.38 (m, 1H), 3.31-3.07 (m, 2H), 2.78-2.59 (m, 1H), 2.25-2.09 (m, 3H), 2.09-1.88 (m, 2H), 1.88-1.48 (m, 2H), 1.28-0.97 (m, 12H). m/z: ES⁺ [M+H]⁺=498.60.

### Example 1.004: N-ethyl-5-fluoro-N-isopropyl-2-((4-((1S,5R)-2-(methyl-L-alanyl)-2,6-diazabicyclo[3.2.1]octan-6-yl)pyrimidin-5-yl)oxy)benzamide

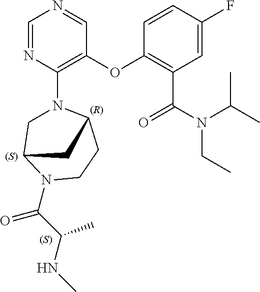

Colourless solid (19 mg, 8% over 2 steps).

¹H NMR (400 MHz, DMSO-d₆, 296 K) (8.35-8.31 (m, 1H), 7.84-7.80 (m, 1H), 7.29-7.22 (m, 2H), 7.10-6.92 (m, 1H), 5.18-4.84 (m, 1H), 4.75-4.30 (m, 1H), 4.27-3.84 (m, 3H), 3.84-3.76 (m, 2H), 3.55-3.46 (m, 1H), 3.29-2.99 (m, 2H), 2.13-3.12 (m, 3H), 2.01-1.69 (m, 3H), 1.68-1.55 (m, 2H), 1.23-0.72 (m, 12H). m/z: ES⁺ [M+H]⁺=499.23.

### Example 1.005: 2-((4-((1S,5R)-2-(dimethyl-D-alanyl)-2,6-diazabicyclo[3.2.1]octan-6-yl)pyrimidin-5-yl)oxy)-N-ethyl-5-fluoro-N-isopropylbenzamide

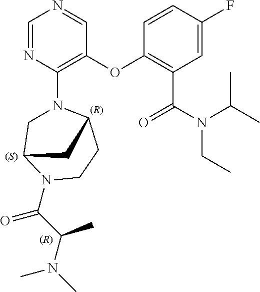

Colourless solid (57 mg, 76%).

¹H NMR (400 MHz, DMSO-d₆, 300 K) δ 8.45-8.26 (m, 1H), 7.88-7.72 (m, 1H), 7.42-7.16 (m, 2H), 7.13-6.85 (m, 1H), 5.26-4.88 (m, 1H), 4.87-4.52 (m, 1H), 4.44-4.02 (m, 1H), 4.01-3.65 (m, 2H), 3.62-3.29 (m, 3H), 3.28-3.01 (m, 2H), 2.91-2.76 (m, 1H), 2.31-2.01 (m, 3H), 2.11-2.04 (m, 3H), 1.93-1.82 (m, 1H), 1.72-1.69 (m, 2H), 1.32-0.91 (m, 12H). m/z: ES⁺ [M+H]⁺=513.63.

### Example 1.006: 2-((4-((1S,5R)-2-(1-aminocyclopropane-1-carbonyl)-2,6-diazabicyclo[3.2.1]octan-6-yl)pyrimidin-5-yl)oxy)-N-ethyl-5-fluoro-N-isopropylbenzamide

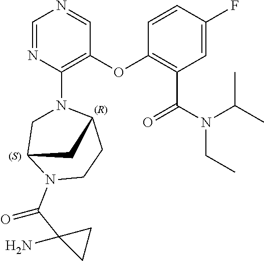

Colourless solid (14 mg, 25%). m/z: ES⁺ [M+H]⁺=497.2.

### Example 1.007: N-ethyl-5-fluoro-N-isopropyl-2-((4-((1S,5R)-2-(1-(methylamino)cyclopropane-1-carbonyl)-2,6-diazabicyclo[3.2.1]octan-6-yl)pyrimidin-5-yl)oxy)benzamide

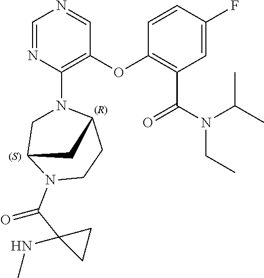

Colourless solid, TFA salt (5 mg, 4% over 2 steps).

¹H NMR (400 MHz, DMSO-d₆, 300 K) δ 8.37-8.30 (m, 1H), 7.94-7.74 (m, 1H), 7.44-7.24 (m, 2H), 7.14-6.98 (m, 1H), 5.46-4.82 (m, 2H), 4.74-4.32 (m, 1H), 4.31-4.05 (m, 1H), 3.95-3.51 (m, 3H), 3.48-3.02 (m, 3H), 2.42-2.15 (m, 3H), 1.97-1.71 (m, 2H), 1.70-1.56 (m, 2H), 1.30-1.02 (m, 9H), 1.92-0.44 (m, 4H). m/z: ES⁺ [M+H]⁺=511.73.

### Example 1.008: 2-((4-((1S,5R)-2-((R)-azetidine-2-carbonyl)-2,6-diazabicyclo[3.2.1]octan-6-yl)pyrimidin-5-yl)oxy)-N-ethyl-5-fluoro-N-isopropylbenzamide

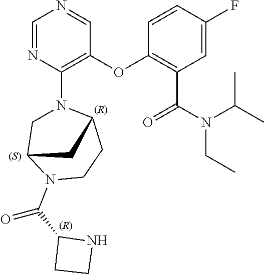

Colourless solid (15 mg, 18% over 2 steps).

¹H NMR (400 MHz, DMSO-d₆, 300 K) δ 8.39-8.25 (m, 1H), 7.90-7.67 (m, 1H), 7.26 (m, 2H), 7.09-6.90 (m, 1H), 5.17-4.56 (m, 2H), 4.48-4.13 (m, 2H), 3.97-3.42 (m, 3H), 3.30-3.07 (m, 4H), 3.07-2.95 (m, 1H), 2.84-2.52 (m, 2H), 2.37-2.11 (m, 1H), 1.86 (s, 2H), 1.85-1.70 (m, 1H), 1.64-1.46 (m, 1H), 1.29-0.90 (m, 9H). m/z: ES⁺ [M+H]⁺=497.20.

### Example 1.009: 2-((4-((1S,5R)-2-((S)-azetidine-2-carbonyl)-2,6-diazabicyclo[3.2.1]octan-6-yl)pyrimidin-5-yl)oxy)-N-ethyl-5-fluoro-N-isopropylbenzamide

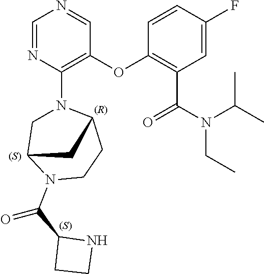

Off-white solid (28 mg, 26%).

¹H NMR (400 MHz, DMSO-d₆, 300 K) δ 8.44-8.19 (m, 1H), 7.96-7.67 (m, 1H), 7.40-7.17 (m, 2H), 7.15-6.89 (m, 1H), 5.20-4.10 (m, 4H), 3.95-3.44 (m, 4H), 3.26-2.89 (m, 4H), 2.60 (s, 1H), 2.38-2.11 (m, 1H), 1.99-1.59 (m, 4H), 1.18-0.98 (m, 9H). m/z: ES⁺ [M+H]⁺=497.20.

### Example 1.010: N-ethyl-5-fluoro-N-isopropyl-2-((4-((1S,5R)-2-((S)-2-methylazetidine-2-carbonyl)-2,6-diazabicyclo[3.2.1]octan-6-yl)pyrimidin-5-yl)oxy)benzamide

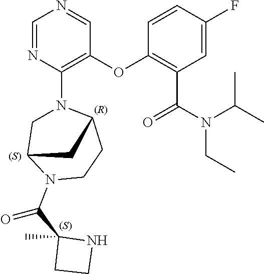

Off-white solid (42 mg, 35%).

¹H NMR (400 MHz, DMSO-d₆, 300 K) δ 8.43-8.14 (m, 1H), 7.97-7.68 (m, 1H), 7.41-7.18 (m, 2H), 7.12-6.97 (m, 1H), 5.24-4.09 (m, 3H), 3.90-3.79 (m, 3H), 3.28-2.91 (m, 6H), 2.72-2.68 (m, 1H), 2.21-2.19 (m, 1H), 1.92-1.87 (m, 2H), 1.77-1.68 (m, 1H), 1.67-1.55 (m, 1H), 1.51-1.30 (m, 3H), 1.20-1.00 (m, 9H). m/z: ES⁺ [M+H]⁺=511.30.

### Example 1.011: N-ethyl-5-fluoro-N-isopropyl-2-((4-((1S,5R)-2-((R)-2-methylazetidine-2-carbonyl)-2,6-diazabicyclo[3.2.1]octan-6-yl)pyrimidin-5-yl)oxy)benzamide

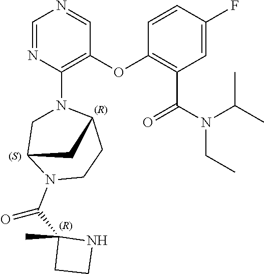

Off-white solid (17 mg, 16%).

¹H NMR (400 MHz, DMSO-d₆, 300 K) δ 8.40-8.24 (m, 1H), 7.91-7.70 (m, 1H), 7.37-7.17 (m, 2H), 7.11-7.01 (m, 1H), 5.23-4.79 (m, 1H), 4.78-4.16 (m, 2H), 4.00-3.54 (m, 3H), 3.30-2.99 (m, 4H), 2.82-2.56 (m, 1H), 2.30-2.04 (m, 1H), 2.03-1.82 (m, 2H), 1.82-1.71 (m, 1H), 1.71-1.50 (m, 2H), 1.47-1.28 (m, 3H), 1.23-0.95 (m, 9H). m/z: ES⁺ [M+H]⁺=511.30.

### Example 1.012: 2-((4-((1S,5R)-2-(D-Prolyl)-2,6-diazabicyclo[3.2.1]octan-6-yl)pyrimidin-5-yl)oxy)-N-ethyl-5-fluoro-N-isopropylbenzamide

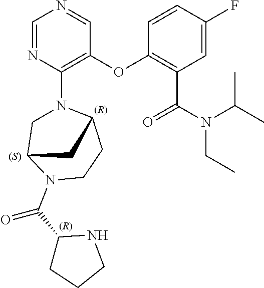

Off-white solid (22 mg, 25% over 2 steps).

¹H NMR (400 MHz, DMSO-d₆, 300 K) δ 8.52-8.24 (m, 1H), 7.95-7.73 (m, 1H), 7.45-7.11 (m, 2H), 7.14-6.78 (m, 1H), 5.23-5.01 (m, 1H), 4.92-4.63 (m, 2H), 4.46-4.02 (m, 1H), 3.92-3.71 (m, 5H), 3.19-3.01 (m, 3H), 3.72-3.63 (m, 1H), 2.42-2.27 (m, 1H), 2.01-1.71 (m, 3H), 1.70-1.51 (m, 3H), 1.43-0.63 (m, 9H). m/z: ES⁺ [M+H]⁺=511.35.

### Example 1.013: 2-((4-((1S,5R)-2-(L-prolyl)-2,6-diazabicyclo[3.2.1]octan-6-yl)pyrimidin-5-yl)oxy)-N-ethyl-5-fluoro-N-isopropylbenzamide

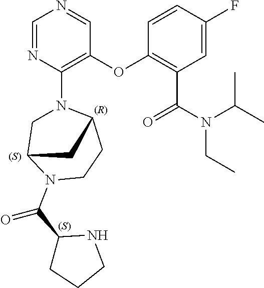

Colourless solid (2 mg, 3% over 2 steps).

¹H NMR (400 MHz, DMSO-d₆, 300 K) δ 8.51-8.13 (m, 1H), 7.85-7.72 (m, 1H), 7.61-7.21 (m, 2H), 7.20-6.76 (m, 1H), 5.24-4.32 (m, 2H), 4.25-3.91 (m, 1H), 3.90-3.65 (m, 3H), 3.22-2.97 (m, 3H), 2.88-2.72 (m, 1H), 2.28-1.81 (m, 3H), 1.80-1.45 (m, 6H), 1.32-0.96 (m, 11H). m/z: ES⁺ [M+H]⁺=511.73.

### Example 1.014: N-ethyl-5-fluoro-N-isopropyl-2-((4-((1S,5R)-2-(methyl-D-prolyl)-2,6-diazabicyclo[3.2.1]octan-6-yl)pyrimidin-5-yl)oxy)benzamide

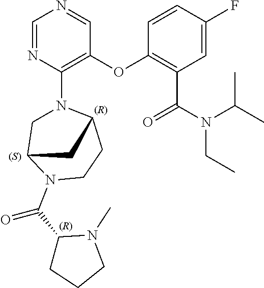

Colourless solid (24 mg, 23%).

¹H NMR (400 MHz, DMSO-d₆, 300 K) δ 8.39-8.25 (m, 1H), 7.87-7.70 (m, 1H), 7.34-7.17 (m, 2H), 7.08-6.86 (m, 1H), 5.20-5.03 (m, 1H), 4.92-4.28 (m, 1H), 4.25-4.00 (m, 1H), 3.95-3.37 (m, 3H), 3.31-3.01 (m, 3H), 3.00-2.75 (m, 1H), 2.75-2.60 (m, 1H), 2.30-2.10 (m, 4H), 2.09-1.97 (m, 1H), 1.95-1.80 (m, 5H), 1.65-1.50 (m, 2H), 1.25-0.87 (m, 9H). m/z: ES⁺ [M+H]⁺=525.30.

### Example 1.015: N-ethyl-5-fluoro-N-isopropyl-2-((4-((1S,5R)-2-(methyl-L-prolyl)-2,6-diazabicyclo[3.2.1]octan-6-yl)pyrimidin-5-yl)oxy)benzamide

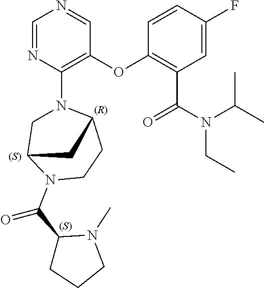

Yellow solid (33 mg, 48%).

¹H NMR (400 MHz, DMSO-d₆, 300 K) δ 8.40-8.31 (m, 1H), 7.41-7.25 (m, 1H), 7.12-6.95 (m, 1H), 5.21-4.95 (m, 1H), 4.92-4.25 (m, 1H), 4.11-3.5 (m, 3H), 3.40-3.31 (m, 1H), 3.30-3.05 (m, 2H), 2.80-2.62 (m, 1H), 2.31-2.13 (m, 4H), 2.11-1.91 (m, 1H), 1.92-1.82 (m, 1H), 1.82-1.45 (m, 5H), 1.21-0.80 (m, 9H). m/z: ES⁺ [M+H]⁺=525.2.

### Example 1.016: N-ethyl-5-fluoro-N-isopropyl-2-((4-((1S,5R)-2-((R)-5-oxopyrrolidine-2-carbonyl)-2,6-diazabicyclo[3.2.1]octan-6-yl)pyrimidin-5-yl)oxy)benzamide

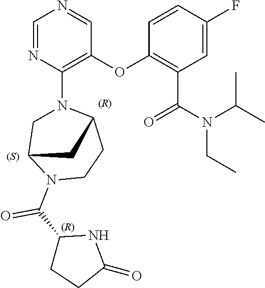

Colourless solid (18 mg, 52%).

m/z: ES⁺ [M+H]⁺=525.3.

### Example 1.017: N-ethyl-5-fluoro-N-isopropyl-2-((4-((1S,5R)-2-((S)-5-oxopyrrolidine-2-carbonyl)-2,6-diazabicyclo[3.2.1]octan-6-yl)pyrimidin-5-yl)oxy)benzamide

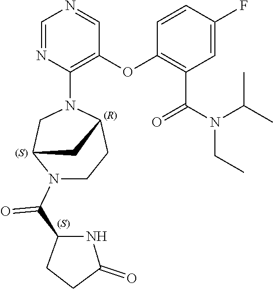

Colourless solid (18 mg, 53%).

m/z: ES⁺ [M+H]⁺=525.3.

### Example 1.018: N-ethyl-5-fluoro-N-isopropyl-2-((4-((1S,5R)-2-((2S,3R)-3-methoxypyrrolidine-2-carbonyl)-2,6-diazabicyclo[3.2.1]octan-6-yl)pyrimidin-5-yl)oxy)benzamide

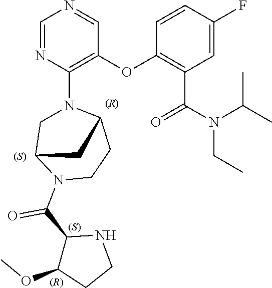

Yellow solid (52 mg, 48% over 2 steps).

¹H NMR (400 MHz, DMSO-d₆, 300 K) δ 8.51-8.14 (m, 1H), 8.10-7.54 (m, 1H), 7.41-7.15 (m, 2H), 7.10-6.64 (m, 1H), 5.40-4.95 (m, 1H), 4.90-4.71 (m, 1H), 4.70-4.22 (m, 1H), 4.12-3.50 (m, 5H), 3.42-3.37 (m, 1H), 3.30-3.18 (m, 1H), 3.17-2.90 (m, 5H), 2.71-2.52 (m, 2H), 2.06-1.42 (m, 6H), 1.27-0.85 (m, 9H). m/z: ES⁺ [M+H]⁺=541.25.

### Example 1.019: N-ethyl-5-fluoro-N-isopropyl-2-((4-((1S,5R)-2-((2S,3S)-3-methoxypyrrolidine-2-carbonyl)-2,6-diazabicyclo[3.2.1]octan-6-yl)pyrimidin-5-yl)oxy)benzamide

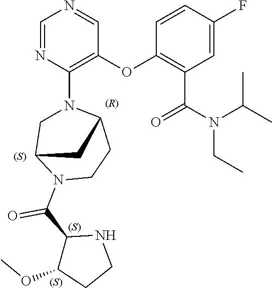

Colourless solid (47 mg, 47% over 2 steps).

¹H NMR (400 MHz, DMSO-d₆, 300 K) δ 8.35-8.25 (m, 1H), 7.90-7.70 (m, 1H), 7.34-7.15 (m, 2H), 7.15-6.93 (m, 1H), 5.20-4.14 (m, 3H), 3.98-3.52 (m, 6H), 3.45-3.38 (m, 2H), 3.35-3.25 (m, 2H), 3.21-3.09 (m, 3H), 3.05-2.98 (m, 1H), 2.85-2.71 (m, 1H), 2.02-1.75 (m, 3H), 1.75-1.55 (m, 2H), 1.55-1.47 (m, 1H), 1.26-0.92 (m, 9H). m/z: ES⁺ [M+H]⁺=541.25.

### Example 1.020: N-ethyl-5-fluoro-2-((4-((1S,5R)-2-((2S,4R)-4-hydroxypyrrolidine-2-carbonyl)-2,6-diazabicyclo[3.2.1]octan-6-yl)pyrimidin-5-yl)oxy)-N-isopropylbenzamide

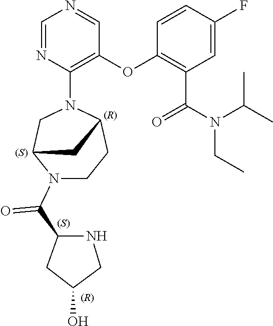

Colourless solid (34 mg, 34% over 2 steps).

¹H NMR (400 MHz, DMSO-d₆, 300 K) δ 8.45-8.33 (m, 1H), 7.87-7.68 (m, 1H), 7.35-7.20 (m, 2H), 7.16-6.89 (m, 1H), 5.17-4.57 (m, 3H), 4.28-3.91 (m, 3H), 3.91-3.52 (m, 4H), 3.45-3.38 (m, 1H), 3.27-2.95 (m, 3H), 2.80-2.54 (m, 1H), 2.00-1.52 (m, 6H), 1.24-0.91 (m, 9H). m/z: ES⁺ [M+H]⁺=527.20.

### Example 1.021: N-ethyl-5-fluoro-2-((4-((1S,5R)-2-((2S,4S)-4-hydroxypyrrolidine-2-carbonyl)-2,6-diazabicyclo[3.2.1]octan-6-yl)pyrimidin-5-yl)oxy)-N-isopropylbenzamide

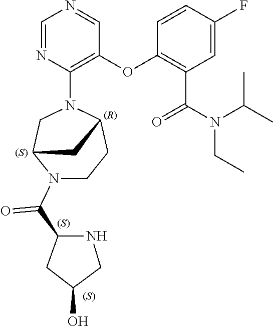

Off-white solid (21 mg, 39% over 2 steps).

1H NMR (400 MHz, DMSO-d₆, 300 K) δ 8.40-8.28 (m, 1H), 8.25-8.15 (m, 1H), 7.81-7.72 (m, 1H), 7.35-7.20 (m, 2H), 7.15-6.95 (m, 1H), 5.25-5.10 (m, 1H), 4.85-4.75 (m, 2H), 4.25-4.10 (m, 1H), 3.95-3.70 (m, 4H), 3.25-3.05 (m, 3H), 2.90-2.80 (m, 2H), 2.35-2.10 (m, 2H), 1.78-1.63 (m, 2H), 1.58-1.42 (m, 3H), 1.25-1.00 (m, 9H). m/z: ES⁺ [M+H]⁺=527.35.

### Example 1.022: N-ethyl-5-fluoro-2-((4-((1S,5R)-2-((2S,3R)-4-hydroxypyrrolidine-2-carbonyl)-2,6-diazabicyclo[3.2.1]octan-6-yl)pyrimidin-5-yl)oxy)-N-isopropylbenzamide

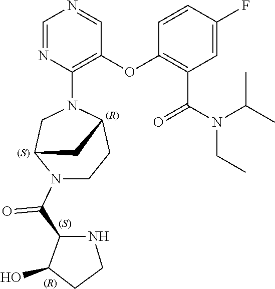

Colourless solid (85 mg, 45% over 2 steps).

¹H NMR (400 MHz, DMSO-d₆, 300 K) δ 8.51-8.08 (m, 1H), 8.03-7.53 (m, 1H), 7.52-7.13 (m, 2H), 7.12-6.63 (m, 1H), 5.35-4.49 (m, 3H), 4.47-4.17 (m, 2H), 4.04-3.50 (m, 5H), 3.42-3.38 (m, 1H), 3.25-2.98 (m, 3H), 2.81-2.58 (m, 1H), 2.01-1.78 (m, 3H), 1.72-1.43 (m, 3H), 1.33-0.88 (m, 9H). m/z: ES⁺ [M+H]⁺=527.25.

### Example 1.023: N-ethyl-5-fluoro-2-((4-((1S,5R)-2-((2RS,3RS)-3-hydroxypyrrolidine-2-carbonyl)-2,6-diazabicyclo[3.2.1]octan-6-yl)pyrimidin-5-yl)oxy)-N-isopropylbenzamide

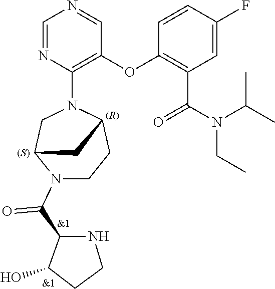

Colourless solid (90 mg, 53% over 2 steps).

¹H NMR (400 MHz, DMSO-d₆, 300 K) δ 8.41-8.22 (m, 1H), 7.91-7.63 (m, 1H), 7.39-7.19 (m, 2H), 7.18-6.89 (m, 1H), 5.40-4.99 (m, 2H), 4.92-4.79 (m, 1H), 4.78-4.25 (m, 1H), 4.22-3.89 (m, 2H), 3.83-3.62 (m, 2H), 3.54-3.38 (m, 1H), 3.28-3.19 (m, 1H), 3.15-2.99 (m, 2H), 2.98-2.72 (m, 2H), 2.70-2.53 (m, 1H), 2.21-2.02 (m, 1H), 1.99-1.81 (m, 2H), 1.79-1.49 (m, 2H), 1.29-0.95 (m, 9H). m/z: ES⁺ [M+H]⁺=529.25.

### Example 1.024: N-ethyl-5-fluoro-2-((4-((1S,5R)-2-((2S,4S)-4-fluoropyrrolidine-2-carbonyl)-2,6-diazabicyclo[3.2.1]octan-6-yl)pyrimidin-5-yl)oxy)-N-isopropylbenzamide and N-ethyl-5-fluoro-2-((4-((1S,5R)-2-((2R,4R)-4-fluoropyrrolidine-2-carbonyl)-2,6-diazabicyclo[3.2.1]octan-6-yl)pyrimidin-5-yl)oxy)-N-isopropylbenzamide

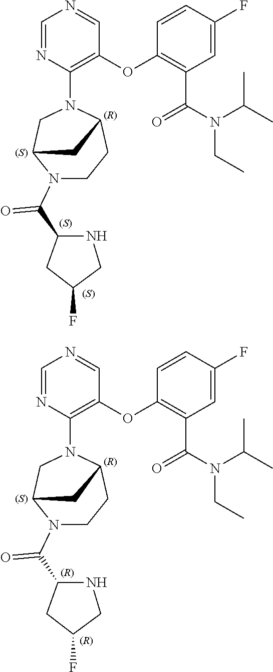

Colourless solid (39 mg, 29% over 2 steps).

¹H NMR (400 MHz, DMSO-d₆, 300 K) δ 8.39-8.28 (m, 1H), 7.91-7.71 (m, 1H), 7.28-7.2 (m, 2H), 7.14-6.91 (m, 1H), 5.49-4.99 (m, 2H), 4.91-4.46 (m, 2H), 4.25-3.61 (m, 6H), 3.21-2.98 (m, 1H), 3.15-2.91 (m, 2H), 2.81-2.79 (m, 1H), 2.88-2.70 (m, 1H), 1.97-1.48 (m, 6H), 1.20-1.00 (m, 9H). m/z: ES⁺ [M+H]⁺=527.25.

### Example 1.025: N-ethyl-5-fluoro-2-((4-((1S,5R)-2-((2S,4R)-4-fluoropyrrolidine-2-carbonyl)-2,6-diazabicyclo[3.2.1]octan-6-yl)pyrimidin-5-yl)oxy)-N-isopropylbenzamide and N-ethyl-5-fluoro-2-((4-((1S,5R)-2-((2R,4S)-4-fluoropyrrolidine-2-carbonyl)-2,6-diazabicyclo[3.2.1]octan-6-yl)pyrimidin-5-yl)oxy)-N-isopropylbenzamide

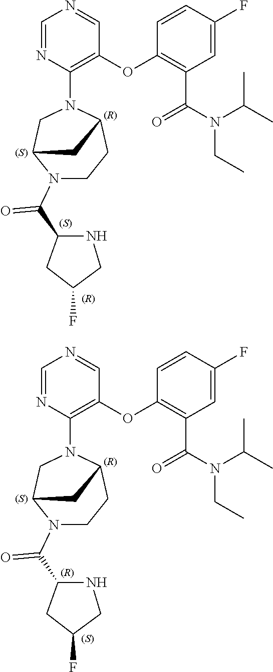

Colourless solid (90 mg, 53% over 2 steps).

¹H NMR (400 MHz, DMSO-d₆, 300 K) δ 8.41-8.22 (m, 1H), 7.91-7.63 (m, 1H), 7.39-7.19 (m, 2H), 7.18-6.89 (m, 1H), 5.40-4.99 (m, 2H), 4.92-4.79 (m, 1H), 4.78-4.25 (m, 1H), 4.22-3.89 (m, 2H), 3.83-3.62 (m, 2H), 3.54-3.38 (m, 1H), 3.28-3.19 (m, 1H), 3.15-2.99 (m, 2H), 2.98-2.72 (m, 2H), 2.70-2.53 (m, 1H), 2.21-2.02 (m, 1H), 1.99-1.81 (m, 2H), 1.79-1.49 (m, 2H), 1.29-0.95 (m, 9H). m/z: ES⁺ [M+H]⁺=529.25.

### Example 1.026: N-ethyl-5-fluoro-2-((4-((1S,5R)-2-((2R,3S)-3-fluoropyrrolidine-2-carbonyl)-2,6-diazabicyclo[3.2.1]octan-6-yl)pyrimidin-5-yl)oxy)-N-isopropylbenzamide

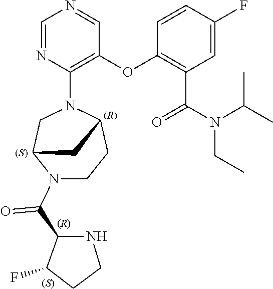

Colourless solid (100 mg, 55% over 2 steps).

¹H NMR (400 MHz, DMSO-d₆, 300 K) δ 8.40-8.24 (m, 1H), 8.20 (s, 1H), 7.91-7.69 (m, 1H), 7.35-7.20 (m, 2H), 7.20-6.95 (m, 1H), 5.49-5.22 (m, 1H), 5.20-5.05 (m, 1H), 5.02-4.54 (m, 1H), 4.46-4.02 (m, 2H), 3.98-3.72 (m, 4H), 3.28-2.95 (m, 4H), 2.85-2.75 (m, 2H), 2.30-2.15 (m, 1H), 2.11-1.56 (m, 4H), 1.41-0.63 (m, 9H). m/z: ES⁺ [M+H]⁺=574.63.

### Example 1.027: N-ethyl-5-fluoro-2-((4-((1S,5R)-2-((2R,3R)-3-fluoropyrrolidine-2-carbonyl)-2,6-diazabicyclo[3.2.1]octan-6-yl)pyrimidin-5-yl)oxy)-N-isopropylbenzamide

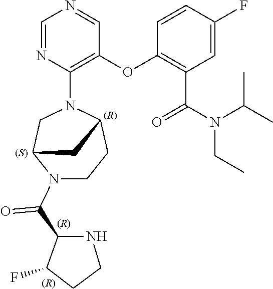

Colourless solid (38 mg, 36% over 2 steps).

¹H NMR (400 MHz, DMSO-d₆, 300 K) δ 8.40-8.28 (m, 1H), 7.95-7.80 (m, 1H), 7.31-7.21 (m, 2H), 7.10-6.95 (m, 1H), 5.55-5.35 (m, 1H), 5.35-5.15 (m, 1H), 5.00-4.90 (m, 1H), 4.87-4.25 (m, 1H), 4.25-4.00 (m, 2H), 4.00-3.80 (m, 3H), 3.30-3.10 (m, 3H), 2.90-2.75 (m, 2H), 2.10-1.90 (m, 4H), 1.90-1.80 (m, 2H), 1.25-0.98 (m, 10H). m/z: ES⁺ [M+H]⁺=574.60.

### Example 1.028: 2-((4-((1S,5R)-2-((1R,3S,5R)-2-azabicyclo[3.1.0]hexane-3-carbonyl)-2,6-diazabicyclo[3.2.1]octan-6-yl)pyrimidin-5-yl)oxy)-N-ethyl-5-fluoro-N-isopropylbenzamide

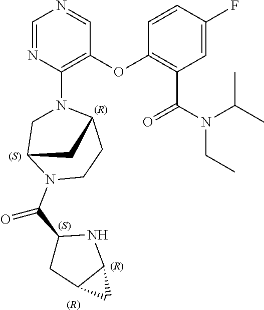

Colourless solid (114 mg, 71% over 2 steps).

¹H NMR (400 MHz, DMSO-d₆, 300 K) δ 8.35-8.33 (m, 1H), 7.89-7.69 (m, 1H), 7.29-7.27 (m, 2H), 7.11-6.91 (m, 1H), 5.19 (s, 1H), 4.81-4.16 (m, 2H), 3.94-3.53 (m, 5H), 3.49-3.38 (m, 1H), 3.28-3.02 (m, 2H), 2.93-2.78 (m, 1H), 2.16-1.76 (m, 3H), 1.72-1.48 (m, 3H), 1.37-1.32 (m, 1H), 1.08-0.98 (m, 9H), 0.51-0.30 (m, 2H). m/z: ES⁺ [M+H]⁺=523.20.

### Example 1.029: 2-((4-((1S,5R)-2-((1S,3S,5S)-2-azabicyclo[3.1.0]hexane-3-carbonyl)-2,6-diazabicyclo[3.2.1]octan-6-yl)pyrimidin-5-yl)oxy)-N-ethyl-5-fluoro-N-isopropylbenzamide

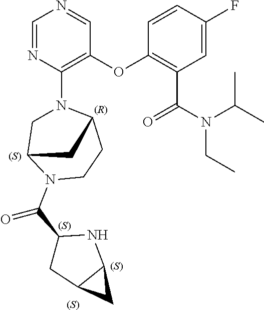

Colourless solid (34 mg, 20% over 2 steps).

¹H NMR (400 MHz, DMSO-d₆, 300 K) δ 8.32-8.30 (m, 1H), 7.84-7.77 (m, 1H), 7.29-7.23 (m, 2H), 7.13-6.93 (m, 1H), 5.15-4.60 (m, 2H), 4.60-4.19 (m, 2H), 3.80-3.65 (m, 3H), 3.27-3.18 (m, 3H), 2.62-2.61 (m, 2H), 2.42-2.31 (m, 1H), 2.00-1.70 (m, 3H), 1.70-1.52 (m, 2H), 1.42-1.28 (m, 1H), 1.26-0.94 (m, 9H), 0.48-0.46 (m, 1H), 0.25-0.16 (m, 1H). m/z: ES⁺ [M+H]⁺=523.33.

### Example 1.030: 2-((4-((1S,5R)-2-((1R,2S,5S)-3-azabicyclo[3.1.0]hexane-2-carbonyl)-2,6-diazabicyclo[3.2.1]octan-6-yl)pyrimidin-5-yl)oxy)-N-ethyl-5-fluoro-N-isopropylbenzamide

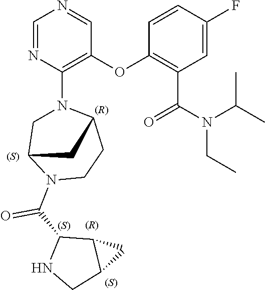

Off-white solid (53 mg, 38% over 2 steps).

¹H NMR (400 MHz, DMSO-d₆, 300 K) δ 8.41-8.11 (m, 1H), 7.88-7.60 (m, 1H), 7.37-7.19 (m, 2H), 7.18-6.59 (m, 1H), 5.24-4.52 (m, 2H), 4.50-4.11 (m, 1H), 4.11-3.88 (m, 2H), 3.87-3.70 (m, 2H), 3.69-3.44 (m, 1H), 3.44-3.32 (m, 1H), 3.30-3.03 (m, 2H), 3.02-2.88 (m, 1H), 2.87-2.70 (m, 1H), 2.11-1.80 (m, 2H), 1.80-1.47 (m, 3H), 1.42-1.27 (m, 1H), 1.25-0.91 (m, 9H), 0.40-0.05 (m, 2H). m/z: ES⁺ [M+H]⁺=523.3.

### Example 1.031: 2-((4-((1S,5R)-2-((1S,2S,5R)-3-azabicyclo[3.1.0]hexane-2-carbonyl)-2,6-diazabicyclo[3.2.1]octan-6-yl)pyrimidin-5-yl)oxy)-N-ethyl-5-fluoro-N-isopropylbenzamide

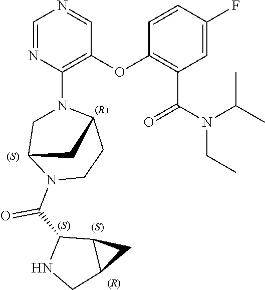

Colourless solid (29 mg, 35% over 2 steps).

¹H NMR (400 MHz, DMSO-d₆, 300 K) δ 8.41-8.32 (m, 1H), 7.95-7.65 (m, 1H), 7.21-6.84 (m, 3H), 5.22-4.25 (m, 2H), 4.18-3.49 (m, 6H), 3.28-3.01 (m, 2H), 2.96-2.63 (m, 2H), 2.96-2.63 (m, 2H), 2.17-1.80 (m, 2H), 1.75-1.29 (m, 4H), 1.24-0.98 (m, 10H), 0.55-0.18 (m, 2H). m/z: ES⁺ [M+H]⁺=523.35.

### Example 1.032: 2-((4-((1S,5R)-2-((S)-5-azaspiro[2.4]heptane-6-carbonyl)-2,6-diazabicyclo[3.2.1]octan-6-yl)pyrimidin-5-yl)oxy)-N-ethyl-5-fluoro-N-isopropylbenzamide

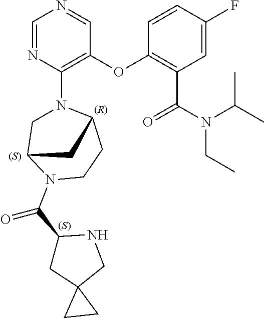

Colourless solid (69 mg, 68% over 2 steps).

¹H NMR (400 MHz, DMSO-d₆, 300 K) δ 8.45-8.33 (m, 1H), 7.91-7.72 (m, 1H), 7.35-7.27 (m, 2H), 7.13-6.84 (m, 1H), 5.21-4.61 (m, 2H), 4.30-4.00 (m, 2H), 3.91-3.58 (m, 4H), 3.25-2.98 (m, 2H), 2.90-2.77 (m, 1H), 2.75-2.60 (m, 1H), 2.06-1.81 (m, 3H), 1.74-1.47 (m, 3H), 1.24-0.91 (m, 10H), 0.64-0.29 (m, 4H). m/z: ES⁺ [M+H]⁺=537.30.

### Example 1.033: 2-((4-((1S,5R)-2-((2S,4S)-4-(difluoromethyl)pyrrolidine-2-carbonyl)-2,6-diazabicyclo[3.2.1]octan-6-yl)pyrimidin-5-yl)oxy)-N-ethyl-5-fluoro-N-isopropylbenzamide

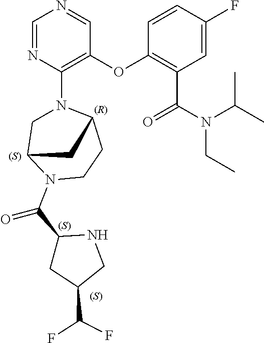

Colourless solid (69 mg, 43% over 2 steps).

¹H NMR (400 MHz, DMSO-d₆, 300 K) δ 8.39-8.26 (m, 1H), 7.88-7.71 (m, 1H), 7.34-7.18 (m, 2H), 7.11-6.81 (m, 1H), 5.93-6.1 (m, 1H), 5.14 (s, 1H), 4.94-4.62 (m, 1H), 4.53-4.09 (m, 1H), 4.00-3.83 (m, 4H), 3.77-3.59 (m, 1H), 3.40-3.37 (m, 1H), 3.32-3.22 (m, 1H), 3.15-3.03 (m, 1H), 2.97-2.86 (m, 1H), 2.87-2.61 (m, 2H), 2.20-2.05 (m, 1H), 1.88-1.75 (m, 2H), 1.59-1.28 (m, 3H), 1.26-1.08 (m, 5H), 1.04-1.01 (m, 4H). m/z: ES⁺ [M+H]⁺=561.30.

### Example 1.034: 2-((4-((1S,5R)-2-((S)-4,4-difluoropyrrolidine-2-carbonyl)-2,6-diazabicyclo[3.2.1]octan-6-yl)pyrimidin-5-yl)oxy)-N-ethyl-5-fluoro-N-isopropylbenzamide

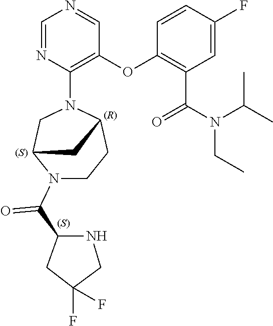

Colourless solid (57 mg, 75% over 2 steps).

¹H NMR (400 MHz, DMSO-d₆, 300 K) δ 8.33-8.31 (m, 1H), 7.85-7.78 (m, 1H), 7.29-7.27 (m, 2H), 7.18-6.80 (m, 1H), 6.07 (br s, 1H), 5.21-5.04 (m, 1H), 4.90-4.70 (m, 1H), 4.68-4.32 (m, 1H), 4.31-4.14 (m, 1H), 3.80-3.76 (m, 4H), 3.31-3.29 (m, 1H), 3.25-2.93 (m, 1H), 2.83-2.70 (m, 1H), 2.54-2.49 (m, 1H), 2.28-2.15 (m, 1H), 2.01-1.82 (s, 2H), 1.81-1.71 (m, 1H), 1.75-1.64 (m, 2H), 1.17-0.98 (m, 9H). m/z: ES⁺ [M+H]⁺=547.2.

### Example 1.035: 2-((4-((1S,5R)-2-((R)-4,4-difluoropyrrolidine-2-carbonyl)-2,6-diazabicyclo[3.2.1]octan-6-yl)pyrimidin-5-yl)oxy)-N-ethyl-5-fluoro-N-isopropylbenzamide and 2-((4-((1S,5R)-2-((S)-4,4-difluoropyrrolidine-2-carbonyl)-2,6-diazabicyclo[3.2.1]octan-6-yl)pyrimidin-5-yl)oxy)-N-ethyl-5-fluoro-N-isopropylbenzamide

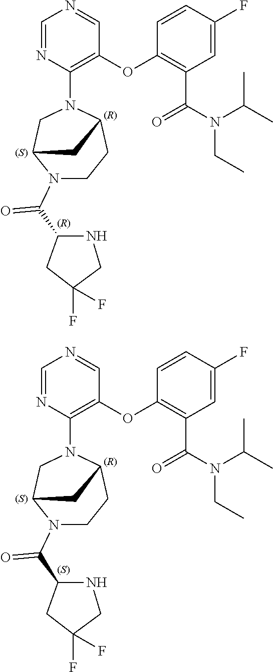

Colourless solid (21 mg, 35%).

m/z: ES⁺ [M+H]⁺=547.3.

### Example 1.036: N-ethyl-5-fluoro-N-isopropyl-2-((4-((1S,5R)-2-((2S,4S)-4-methylpyrrolidine-2-carbonyl)-2,6-diazabicyclo[3.2.1]octan-6-yl)pyrimidin-5-yl)oxy)benzamide

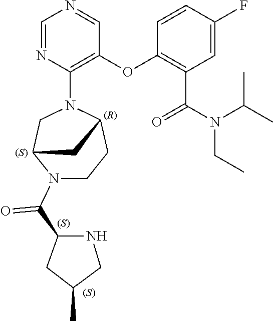

Colourless solid (58 mg, 69% over 2 steps).

¹H NMR (400 MHz, DMSO-d₆, 300 K) δ 8.40-8.25 (m, 1H), 7.90-7.66 (m, 1H), 7.36-7.18 (m, 2H), 7.13-6.88 (m, 1H), 5.19-4.52 (m, 2H), 4.49-4.08 (m, 1H), 3.97-3.57 (m, 4H), 3.55-3.39 (m, 1H), 3.29-2.97 (m, 2H), 2.88-2.64 (m, 2H), 2.48-2.42 (m, 1H), 2.28-1.99 (m, 2H), 1.98-1.80 (m, 2H), 1.75-1.52 (m, 2H), 1.27-0.96 (m, 10H), 0.96-0.82 (m, 3H). m/z: ES⁺ [M+H]⁺=525.40.

### Example 1.037: N-ethyl-5-fluoro-N-isopropyl-2-((4-((1S,5R)-2-((2S,4R)-4-methylpyrrolidine-2-carbonyl)-2,6-diazabicyclo[3.2.1]octan-6-yl)pyrimidin-5-yl)oxy)benzamide

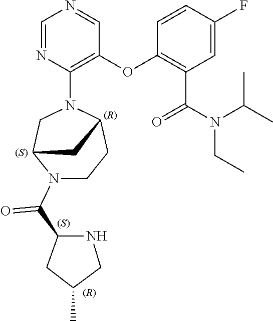

Colourless solid, (53 mg, 45%).

¹H NMR (400 MHz, DMSO-d₆, 300 K) δ 8.43-8.32 (m, 1H), 7.95-7.65 (m, 1H), 7.58-7.16 (m, 2H), 7.14-6.68 (m, 1H), 5.24-4.55 (m, 2H), 4.54-4.02 (m, 1H), 4.01-3.52 (m, 4H), 3.38-3.32 (m, 1H), 3.31-2.62 (m, 3H), 2.34-2.08 (m, 1H), 2.05-1.78 (m, 3H), 1.76-1.36 (m, 4H), 1.26-1.15 (m, 4H), 1.14-0.98 (m, 4H), 1.12-0.88 (m, 3H). m/z: ES⁺ [M+H]⁺=525.3.

### Example 1.038: N-ethyl-5-fluoro-N-isopropyl-2-((4-((1S,5R)-2-((2S,5R)-5-methylpyrrolidine-2-carbonyl)-2,6-diazabicyclo[3.2.1]octan-6-yl)pyrimidin-5-yl)oxy)benzamide

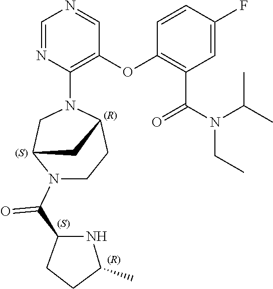

Colourless solid (58 mg, 69% over 2 steps).

¹H NMR (400 MHz, DMSO-d₆, 300 K) δ 8.40-8.25 (m, 1H), 7.90-7.66 (m, 1H), 7.36-7.18 (m, 2H), 7.13-6.88 (m, 1H), 5.19-4.52 (m, 2H), 4.49-4.08 (m, 1H), 3.97-3.57 (m, 4H), 3.55-3.39 (m, 1H), 3.29-2.97 (m, 2H), 2.88-2.64 (m, 2H), 2.48-2.42 (m, 1H), 2.28-1.99 (m, 2H), 1.98-1.80 (m, 2H), 1.75-1.52 (m, 2H), 1.27-0.96 (m, 10H), 0.96-0.82 (m, 3H). m/z: ES⁺ [M+H]⁺=525.40.

### Example 1.039: N-ethyl-5-fluoro-N-isopropyl-2-((4-((1S,5R)-2-((2S,5S)-5-methylpyrrolidine-2-carbonyl)-2,6-diazabicyclo[3.2.1]octan-6-yl)pyrimidin-5-yl)oxy)benzamide

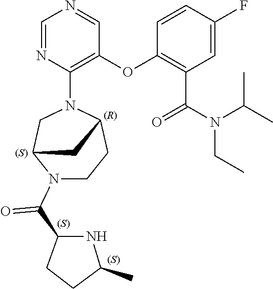

Yellow solid, hydrochloride salt (80 mg, 49% over 2 steps).

¹H NMR (400 MHz, DMSO-d₆, 300 K) δ 8.45-8.12 (m, 1H), 7.89-7.65 (m, 1H), 7.36-7.11 (m, 2H), 7.06-6.84 (m, 1H), 5.20-4.56 (m, 2H), 4.45-3.49 (m, 5H), 3.25-3.00 (m, 2H), 2.98-2.80 (m, 1H), 2.87-2.56 (m, 1H), 2.21-2.02 (m, 1H), 2.02-1.73 (m, 3H), 1.82-1.42 (m, 4H), 1.22-0.93 (m, 12H). m/z: ES⁺ [M+H]⁺=525.2.

### Example 1.040: N-ethyl-5-fluoro-N-isopropyl-2-((4-((1S,5R)-2-((2S,3S)-3-methylpyrrolidine-2-carbonyl)-2,6-diazabicyclo[3.2.1]octan-6-yl)pyrimidin-5-yl)oxy)benzamide

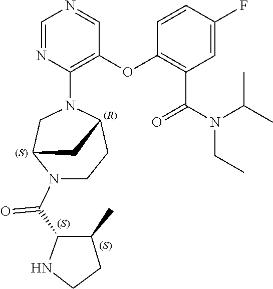

Off-white solid, formic acid salt (39 mg, 18% over 2 steps).

¹H NMR (400 MHz, DMSO-d₆, 300 K) δ 8.39-8.28 (m, 2H), 7.89-7.76 (m, 3H), 7.14-6.99 (m, 1H), 5.19-5.09 (m, 1H), 4.91-4.74 (m, 2H), 4.00-3.53 (m, 5H), 3.48-3.30 (m, 1H), 3.30-2.70 (m, 4H), 2.37-2.10 (m, 1H), 2.04-1.56 (m, 5H), 1.55-1.38 (m, 1H), 1.30-0.80 (m, 11H). m/z: ES⁺ [M+H]⁺=525.2.

### Example 1.041: N-ethyl-5-fluoro-N-isopropyl-2-((4-((1S,5R)-2-((2S,3R)-3-methylpyrrolidine-2-carbonyl)-2,6-diazabicyclo[3.2.1]octan-6-yl)pyrimidin-5-yl)oxy)benzamide

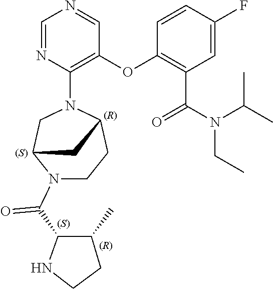

Colourless solid (41 mg, 35% over 2 steps).

¹H NMR (400 MHz, DMSO-d₆, 300 K) δ 8.34 (t, J=10.7 Hz, 1H), 7.89-7.75 (m, 1H), 7.33-7.14 (m, 2H), 7.13-6.82 (m, 1H), 5.20 (q, J=4.3 Hz, 1H), 4.96-4.34 (m, 2H), 4.30-3.52 (m, 5H), 3.28-2.93 (m, 4H), 2.79-2.57 (m, 1H), 2.44-2.22 (m, 1H), 2.02-1.81 (m, 3H), 1.66 (q, J=10.7, 7.5 Hz, 2H), 1.45-1.25 (m, 1H), 1.22-0.99 (m, 9H), 0.75-0.57 (m, 2H), 0.51 (s, 1H). m/z: ES⁺ [M+H]⁺=525.3.

### Example 1.042: 2-((4-((1S,5R)-2-(2-azabicyclo[2.1.1]hexane-1-carbonyl)-2,6-diazabicyclo[3.2.1]octan-6-yl)pyrimidin-5-yl)oxy)-N-ethyl-5-fluoro-N-isopropylbenzamide

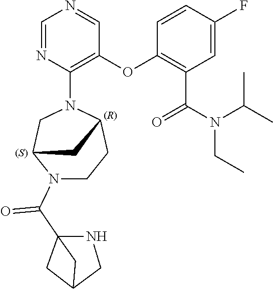

Colourless solid (47 mg, 63% over 2 steps).

¹H NMR (400 MHz, DMSO-d₆, 300 K) δ 8.32 (m, 1H), 7.87-7.72 (m, 1H), 7.35-7.19 (m, 2H), 7.10-6.89 (m, 1H), 5.26-4.58 (m, 2H), 4.23 (m, 1H), 3.96-3.36 (m, 4H), 3.29-2.95 (m, 2H), 2.84 (m, 2H), 2.77-2.62 (m, 1H), 2.57 (m, 1H), 1.96-1.66 (m, 4H), 1.58 (m, 2H), 1.41 (m, 2H), 1.26-0.91 (m, 9H). m/z: ES⁺ [M+H]⁺=523.3.

### Example 1.043: N-ethyl-2-((4-((1S,5R)-2-((S)-2-ethylpyrrolidine-2-carbonyl)-2,6-diazabicyclo[3.2.1]octan-6-yl)pyrimidin-5-yl)oxy)-5-fluoro-N-isopropylbenzamide

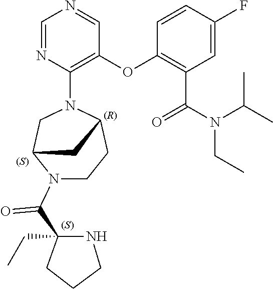

Colourless solid (34 mg, 24% over 2 steps).

¹H NMR (400 MHz, DMSO-d₆, 300 K) δ 8.32 (d, J=2.8 Hz, 1H), 7.98-7.53 (m, 1H), 7.34-7.18 (m, 2H), 7.05-6.81 (m, 1H), 5.18 (s, 1H), 4.93-4.55 (m, 1H), 4.51-4.17 (m, 1H), 4.01-3.50 (m, 3H), 3.43-3.35 (m, 1H), 3.29-2.99 (m, 2H), 2.98-2.81 (m, 1H), 2.80-2.72 (m, 1H), 2.65-2.52 (m, 1H), 2.25-1.80 (m, 3H), 1.78-1.47 (m, 7H), 1.23-1.10 (m, 5H), 1.09-0.98 (m, 4H), 0.81-0.53 (m, 3H). m/z: ES⁺ [M+H]⁺=539.30.

### Example 1.044: N-ethyl-5-fluoro-N-isopropyl-2-((4-((1S,5R)-2-((R)-pyrrolidine-3-carbonyl)-2,6-diazabicyclo[3.2.1]octan-6-yl)pyrimidin-5-yl)oxy)benzamide

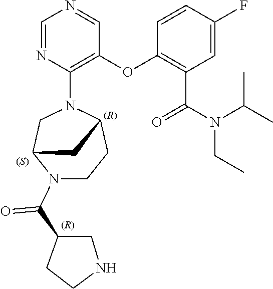

Colourless solid (72 mg, 58% over 2 steps).

¹H NMR (400 MHz, DMSO-d₆, 300 K) δ 8.47-8.18 (m, 1H), 8.00-7.57 (m, 1H), 7.52-7.18 (m, 2H), 7.18-6.75 (m, 1H), 5.21-4.81 (m, 1H), 4.80-4.14 (m, 2H), 3.91-3.69 (m, 3H), 3.68-3.58 (m, 1H), 3.40-3.35 (m, 2H), 3.29-3.20 (m, 2H), 3.18-3.10 (m, 1H), 3.02-2.89 (m, 1H), 2.85-2.64 (m, 2H), 2.08-1.50 (m, 6H), 1.22-0.99 (m, 9H). m/z: ES⁺ [M+H]⁺=511.25.

### Example 1.045: N-ethyl-5-fluoro-N-isopropyl-2-((4-((1S,5R)-2-((S)-pyrrolidine-3-carbonyl)-2,6-diazabicyclo[3.2.1]octan-6-yl)pyrimidin-5-yl)oxy)benzamide

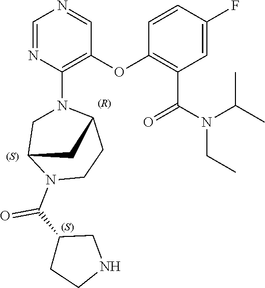

Colourless solid (40 mg, 35% over 2 steps).

¹H NMR (400 MHz, DMSO-d₆, 300 K) δ 8.31-8.04 (m, 1H), 7.95-7.62 (m, 1H), 7.41-7.15 (m, 2H), 7.13-6.85 (m, 1H), 5.38-4.79 (m, 1H), 4.78-4.08 (m, 2H), 3.96-3.53 (m, 4H), 3.46-3.06 (m, 5H), 3.17-3.08 (m, 1H), 2.90-2.65 (m, 2H), 2.16-1.75 (m, 4H), 1.74-1.45 (m, 2H), 1.35-0.85 (m, 9H). m/z: ES⁺ [M+H]⁺=511.30.

### Example 1.046: N-ethyl-5-fluoro-N-isopropyl-2-((4-((1S,5R)-2-((R)-3-methylpyrrolidine-3-carbonyl)-2,6-diazabicyclo[3.2.1]octan-6-yl)pyrimidin-5-yl)oxy)benzamide

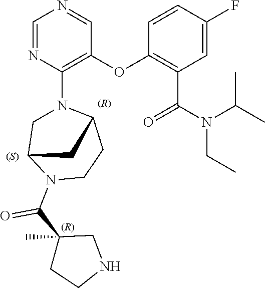

Colourless solid, formic acid salt (68 mg, 49% over 2 steps).

¹H NMR (400 MHz, DMSO-d₆, 300 K) δ 8.60-8.40 (m, 1H), 8.37-8.30 (m, 2H), 7.92-7.84 (m, 1H), 7.34-7.24 (m, 2H), 7.10-6.98 (m, 1H), 4.90-4.60 (m, 2H), 3.98-3.80 (m, 3H), 3.70-3.60 (m, 2H), 3.20-3.00 (m, 3H), 3.00-2.90 (m, 1H), 2.40-2.20 (m, 2H), 1.80-1.60 (m, 2H), 1.40-1.20 (m, 3H), 1.20-1.00 (m, 1H), 1.10-1.00 (m, 4H), 1.00-0.80 (m, 5H). m/z: ES⁺ [M+H]⁺=525.10.

### Example 1.047: N-ethyl-5-fluoro-N-isopropyl-2-((4-((1S,5R)-2-((S)-3-methylpyrrolidine-3-carbonyl)-2,6-diazabicyclo[3.2.1]octan-6-yl)pyrimidin-5-yl)oxy)benzamide

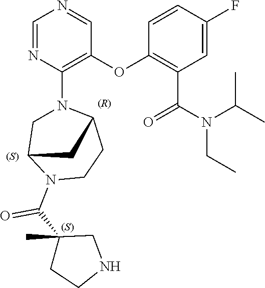

Colourless solid (52 mg, 47% over 2 steps).

¹H NMR (400 MHz, DMSO-d₆, 300 K) δ 8.40-8.30 (m, 2H), 7.85-7.78 (m, 1H), 7.32-7.22 (m, 2H), 7.01-6.99 (m, 1H), 5.10 (s, 1H), 4.85 (s, 1H), 4.30-4.19 (m, 2H), 3.81-3.76 (m, 3H), 3.63-3.54 (m, 2H), 3.41-3.38 (m, 1H), 3.36-3.21 (m, 3H), 3.19-2.67 (m, 2H), 2.27 (s, 1H), 1.89 (s, 2H), 1.76-1.67 (m, 2H), 1.31 (s, 3H), 1.23-0.90 (m, 9H).

m/z: ES⁺ [M+H]⁺=525.29.

### Example 1.048: N-ethyl-5-fluoro-N-isopropyl-2-((4-((1S,5R)-2-((S)-piperidine-2-carbonyl)-2,6-diazabicyclo[3.2.1]octan-6-yl)pyrimidin-5-yl)oxy)benzamide

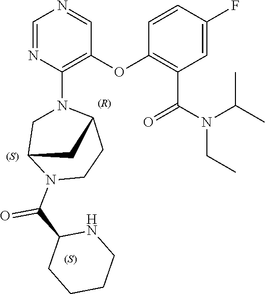

Colourless solid (50 mg, 38% over 2 steps).

¹H NMR (400 MHz, DMSO-d₆, 300 K) δ 8.42-8.18 (m 1H), 8.01-7.61 (m, 1H), 7.45-7.19 (m 2H), 7.19-7.62 (m, 1H), 5.23-4.88 (m 1H), 4.88-4.27 (m, 1H), 4.27-3.74 (m, 3H), 3.74-3.48 (m, 2H), 3.23-3.05 (m, 2H), 3.00-2.79 (m, 1H) 2.79-2.57 (m, 1H), 2.06-1.39 (m, 9H), 1.31-1.00 (m, 11H). m/z: ES⁺ [M+H]⁺=525.30.

### Example 1.049: N-ethyl-5-fluoro-N-isopropyl-2-((4-((1S,5R)-2-((R)-piperidine-2-carbonyl)-2,6-diazabicyclo[3.2.1]octan-6-yl)pyrimidin-5-yl)oxy)benzamide

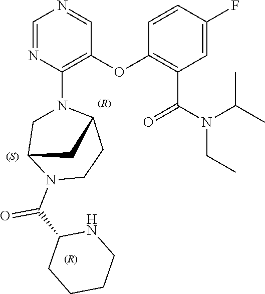

Colourless solid (73 mg, 52% over 2 steps).

¹H NMR (400 MHz, DMSO-d₆, 300 K) δ 8.46-8.18 (m, 1H), 7.95-7.62 (m, 1H), 7.41-7.15 (m, 2H), 7.13-6.85 (m, 1H), 5.28-4.26 (m, 2H), 4.24-3.68 (m, 3H), 3.67-3.36 (m, 3H), 3.27-3.06 (m, 2H), 3.01-2.86 (m, 1H), 2.53-2.41 (m, 1H), 2.03-1.85 (m, 2H), 1.84-1.75 (m, 2H), 1.74-1.45 (m, 5H), 1.44-1.16 (m, 3H), 1.15-0.96 (m, 8H). m/z: ES⁺ [M+H]⁺=525.25.

### Examples 1.050 and 1.051: N-ethyl-5-fluoro-N-isopropyl-2-((4-((1S,5R)-2-((S)-2-methylpiperidine-2-carbonyl)-2,6-diazabicyclo[3.2.1]octan-6-yl)pyrimidin-5-yl)oxy)benzamide and N-ethyl-5-fluoro-N-isopropyl-2-((4-((1S,5R)-2-((R)-2-methylpiperidine-2-carbonyl)-2,6-diazabicyclo[3.2.1]octan-6-yl)pyrimidin-5-yl)oxy)benzamide

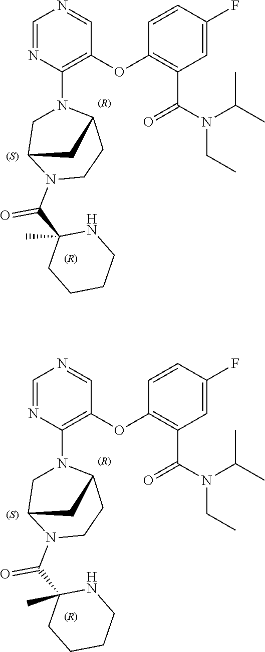

Separation by chiral HPLC gave two single stereoisomers:

Peak #1: off-white solid (18 mg, 7%).

¹H NMR (400 MHz, DMSO-d₆, 300 K) δ 8.50-8.10 (m, 1H), 7.95-7.60 (m, 1H), 7.40-7.15 (m, 2H), 7.03-6.85 (m, 1H), 5.68-3.95 (m, 3H), 3.85-3.55 (m, 3H), 3.45-3.35 (m, 1H), 3.22-3.05 (m, 2H), 3.00-2.65 (m, 2H), 2.40-2.05 (m, 1H), 1.95-1.75 (m, 2H), 1.75-1.58 (m, 3H), 1.58-1.35 (m, 3H), 1.30-1.15 (m, 5H), 1.12-0.93 (m, 9H). m/z: ES⁺ [M+H]⁺=539.30.

Peak #2: colourless solid (9 mg, 4%).

¹H NMR (400 MHz, DMSO-d₆, 300 K) δ 8.55-8.25 (m, 1H), 7.95-7.65 (m, 1H), 7.40-7.15 (m, 2H), 7.13-6.81 (m, 1H), 6.80-4.90 (m, 2H), 4.80-4.28 (m, 2H), 3.95-3.45 (m, 3H), 3.44-3.40 (m, 1H), 3.22-3.14 (m, 1H), 2.98-2.67 (m, 2H), 2.39-2.25 (m, 1H), 2.20-2.09 (m, 1H), 1.95-1.75 (m, 2H), 1.86-1.55 (m, 4H), 1.45-0.93 (m, 15H). m/z: ES⁺ [M+H]⁺=539.25.

### Example 1.052: N-ethyl-5-fluoro-N-isopropyl-2-((4-((1S,5R)-2-((R)-piperazine-2-carbonyl)-2,6-diazabicyclo[3.2.1]octan-6-yl)pyrimidin-5-yl)oxy)benzamide

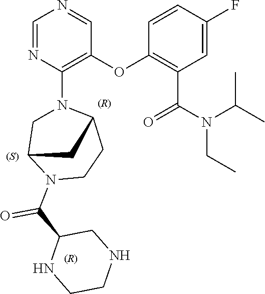

Colourless solid (27 mg, 46%).

m/z: ES⁺ [M+H]⁺=526.3.

### Example 1.053: N-ethyl-5-fluoro-N-isopropyl-2-((4-((1S,5R)-2-((S)-piperazine-2-carbonyl)-2,6-diazabicyclo[3.2.1]octan-6-yl)pyrimidin-5-yl)oxy)benzamide

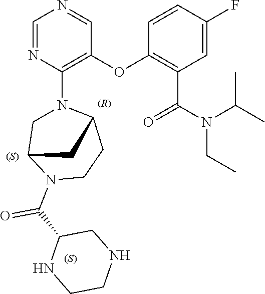

Colourless solid (26 mg, 45%).

m/z: ES⁺ [M+H]⁺=526.3.

### Example 1.054: N-ethyl-5-fluoro-N-isopropyl-2-((4-((1S,5R)-2-((S)-morpholine-3-carbonyl)-2,6-diazabicyclo[3.2.1]octan-6-yl)pyrimidin-5-yl)oxy)benzamide

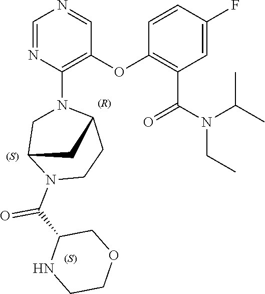

Off-white solid (19 mg, 15% over 2 steps).

¹H NMR (400 MHz, DMSO-d₆, 300 K) δ 8.35-8.25 (m, 1H), 7.86-7.71 (m, 1H), 7.35-7.25 (m, 2H), 7.10-6.85 (m, 1H), 5.15-4.95 (m, 1H), 4.89-4.36 (m, 1H), 4.25-4.05 (m, 2H), 3.9-3.65 (m, 6H), 3.35-3.20 (m, 1H), 3.20-3.10 (m, 3H), 2.80-2.65 (m, 2H), 2.25-2.05 (m, 1H), 2.00-1.80 (m, 2H), 1.80-1.59 (m, 2H), 1.07-0.96 (m, 9H). m/z: ES⁺ [M+H]⁺=527.61.

### Example 1.055: N-ethyl-5-fluoro-N-isopropyl-2-((4-((1S,5R)-2-((R)-morpholine-3-carbonyl)-2,6-diazabicyclo[3.2.1]octan-6-yl)pyrimidin-5-yl)oxy)benzamide

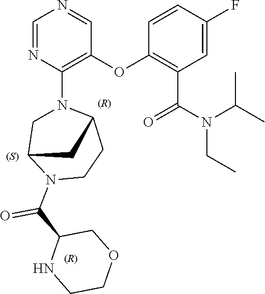

Colourless solid (29 mg, 94% over 2 steps).

¹H NMR (400 MHz, DMSO-d₆, 300 K) δ 8.42-8.19 (m, 1H), 7.93-7.62 (m, 1H), 7.41-7.17 (m, 2H), 7.15-6.87 (m, 1H), 5.18-4.57 (m, 2H), 4.54-4.01 (m, 1H), 3.99-3.70 (m, 4H), 3.70-3.47 (m, 3H), 3.31-3.18 (m, 3H), 3.18-3.08 (m, 1H), 2.83-2.65 (m, 2H), 2.30-2.08 (m, 1H), 2.01-1.83 (m, 2H), 1.78-1.49 (m, 2H), 1.26-1.08 (m, 5H), 1.07-0.96 (m, 4H). m/z: ES⁺ [M+H]⁺=527.30.

### Example 1.056: N-ethyl-5-fluoro-N-isopropyl-2-((4-((1S,5R)-2-((R)-morpholine-2-carbonyl)-2,6-diazabicyclo[3.2.1]octan-6-yl)pyrimidin-5-yl)oxy)benzamide

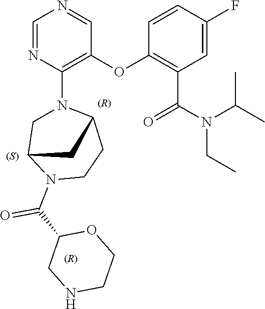

Colourless solid (25 mg, 43%).

m/z: ES⁺ [M+H]⁺=527.3.

### Example 1.057: N-ethyl-5-fluoro-N-isopropyl-2-((4-((1S,5R)-2-((S)-morpholine-2-carbonyl)-2,6-diazabicyclo[3.2.1]octan-6-yl)pyrimidin-5-yl)oxy)benzamide

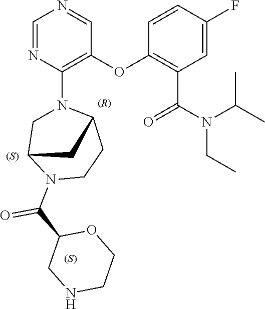

Colourless solid (18 mg, 31%).

m/z: ES⁺ [M+H]⁺=527.3.

### Example 1.058: N-ethyl-5-fluoro-N-isopropyl-2-((4-((1S,5R)-2-((S)-3-methylmorpholine-3-carbonyl)-2,6-diazabicyclo[3.2.1]octan-6-yl)pyrimidin-5-yl)oxy)benzamide

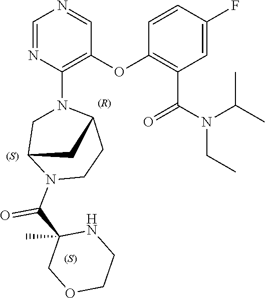

Colourless solid (36 mg, 18% over 2 steps).

¹H NMR (400 MHz, DMSO-d₆, 300 K) δ 8.33 (d, 1H), 7.79 (s, 1H), 7.35-7.20 (m, 2H), 7.08-6.92 (m, 1H), 6.64-5.25 (br s, 1H), 4.61 (br s, 2H), 4.03 (d, 1H), 3.92-3.51 (m, 3H), 3.50-3.30 (m, 3H), 3.28-3.00 (m, 2H), 3.00-2.71 (m, 2H), 2.56-2.46 (m, 2H), 2.00-1.80 (m, 2H), 1.70-1.50 (m, 2H), 1.28-0.88 (m, 12H). m/z: ES⁺ [M+H]⁺=541.25.

### Example 1.059: N-ethyl-5-fluoro-N-isopropyl-2-((4-((1S,5R)-2-((2R,3S)-2-methylmorpholine-3-carbonyl)-2,6-diazabicyclo[3.2.1]octan-6-yl)pyrimidin-5-yl)oxy)benzamide

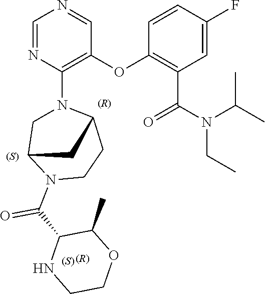

Off-white solid (30 mg, 29% over 2 steps).

¹H NMR (400 MHz, DMSO-d₆, 300 K) δ 8.35-8.25 (m, 1H), 7.90-7.69 (m, 1H), 7.35-7.18 (m, 2H), 7.14-6.88 (m, 1H), 5.18-4.90 (m, 1H), 4.85-4.56 (m, 1H), 4.15-4.00 (m, 1H), 3.94-3.53 (m, 4H), 3.49-3.26 (m, 3H), 3.26-3.10 (m, 1H), 2.80-2.60 (m, 3H), 2.35-1.51 (m, 6H), 1.30-1.10 (m, 1H), 1.14-0.96 (m, 9H), 0.94-0.82 (m, 2H). m/z: ES⁺ [M+H]⁺=540.64.

### Example 1.060: 2-((4-((1S,5R)-2-((S)-6,6-dimethylmorpholine-3-carbonyl)-2,6-diazabicyclo[3.2.1]octan-6-yl)pyrimidin-5-yl)oxy)-N-ethyl-5-fluoro-N-isopropylbenzamide

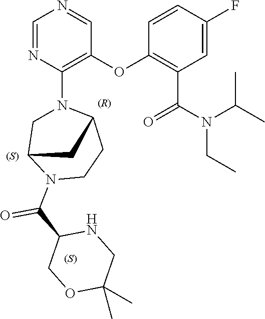

Colourless solid, trifluoroacetic acid salt (47 mg, 42% over 2 steps).

¹H NMR (400 MHz, DMSO-d₆, 300 K) δ 8.33 (m, 1H), 7.92-7.70 (m, 1H), 7.27 (m, 2H), 7.12-6.90 (m, 1H), 5.12 (s, 1H), 4.50 (m, 1H), 4.20-3.93 (m, 1H), 3.90-3.63 (m, 3H), 3.55 (m, 2H), 3.47-3.37 (m, 2H), 3.28-3.01 (m, 2H), 2.71-2.53 (m, 2H), 2.37 (m, 1H), 1.87 (m, 2H), 1.77-1.51 (m, 2H), 1.16 (m, 4H), 1.11 (m, 4H), 1.02 (m, 7H).

m/z: ES⁺ [M+H]⁺=555.3.

### Example 1.061: N-ethyl-5-fluoro-N-isopropyl-2-((4-((1S,5R)-2-(2-morpholinoacetyl)-2,6-diazabicyclo[3.2.1]octan-6-yl)pyrimidin-5-yl)oxy)benzamide

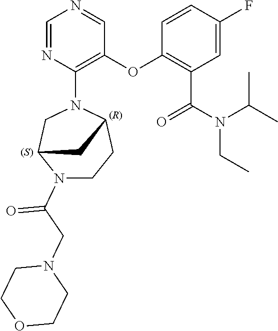

Colourless solid (49 mg, 47%).

¹H NMR (400 MHz, DMSO-d₆, 300 K) δ 8.36-8.22 (m, 1H), 8.01-7.65 (m, 1H), 7.46-7.13 (m, 2H), 7.13-6.64 (m, 1H), 5.11-4.88 (m, 1H), 4.78-4.22 (m, 1H), 4.22-3.90 (m, 2H), 3.90-3.78 (m, 2H), 3.78-3.46 (m, 6H), 3.29-3.12 (m, 2H), 3.12-3.03 (m, 1H), 2.79-2.59 (m, 1H), 2.41-2.39 (m, 3H), 2.17-1.80 (m, 2H), 1.80-1.52 (m, 2H), 1.23-0.98 (m, 9H). m/z: ES⁺ [M+H]⁺=540.64.

### Example 1.062: N-ethyl-5-fluoro-N-isopropyl-2-((4-((1S,5R)-2-((R)-2-methylpyrrolidine-2-carbonyl)-2,6-diazabicyclo[3.2.1]octan-6-yl)pyrimidin-5-yl)oxy)benzamide

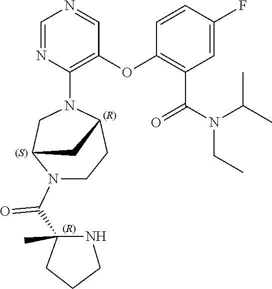

Colourless solid (28 mg, 24% over 2 steps).

¹H NMR (400 MHz, DMSO-d₆, 300 K) δ 8.70-8.30 (m, 1H), 7.88-7.71 (m, 1H), 7.34-7.18 (m, 2H), 7.05-6.90 (m, 1H), 5.50-5.10 (m, 1H), 4.86-4.57 (m, 1H), 4.42-4.10 (m, 1H), 3.95-3.50 (m, 3H), 3.17-3.02 (m, 1H) 2.91-2.59 (m, 4H), 2.23-2.08 (m, 2H), 2.00-1.79 (m, 2H), 1.77-1.42 (m, 5H), 1.38-0.81 (m, 12H). m/z: ES⁺ [M+H]⁺=524.64.

### Example 1.063: N-ethyl-5-fluoro-N-isopropyl-2-((4-((1S,5R)-2-((S)-2-methylpyrrolidine-2-carbonyl)-2,6-diazabicyclo[3.2.1]octan-6-yl)pyrimidin-5-yl)oxy)benzamide

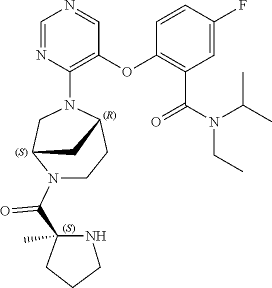

Yellow solid (50 mg, 49% over 2 steps).

¹H NMR (400 MHz, DMSO-d₆, 300 K) δ 8.45-8.33 (m, 1H), 7.93-7.71 (m, 1H), 7.41-7.18 (m, 2H), 7.10-6.95 (m, 1H), 5.40-5.10 (m, 1H), 4.90-4.65 (m, 1H), 4.50-4.15 (m, 2H), 3.93-3.51 (m, 3H), 3.48-3.37 (m, 1H), 3.27-3.06 (m, 2H), 2.95-2.86 (m, 1H), 2.80-2.67 (m, 1H), 2.35-2.15 (m, 1H), 2.00-1.80 (m, 2H), 1.80-1.60 (m, 5H), 1.28 (s, 3H), 1.23-0.94 (m, 9H). m/z: ES⁺ [M+H]⁺=525.3.

### Examples 1.064 and 1.065: 2-((4-((1S,5R)-2-((1R,5S)-2-azabicyclo[3.1.0]hexane-1-carbonyl)-2,6-diazabicyclo[3.2.1]octan-6-yl)pyrimidin-5-yl)oxy)-N-ethyl-5-fluoro-N-isopropylbenzamide and 2-((4-((1S,5R)-2-((1S,5R)-2-azabicyclo[3.1.0]hexane-1-carbonyl)-2,6-diazabicyclo[3.2.1]octan-6-yl)pyrimidin-5-yl)oxy)-N-ethyl-5-fluoro-N-isopropylbenzamide

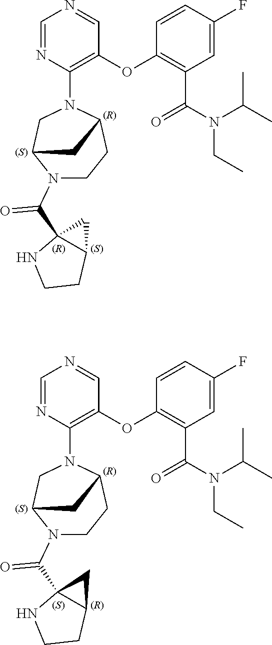

Separation by chiral HPLC gave two single stereoisomers:

Peak #1: colourless solid (24 mg, 3% over 2 steps).

¹H NMR (400 MHz, DMSO-d₆, 300 K) δ 8.43-8.12 (m, 1H), 8.09-7.63 (m, 1H), 7.48-7.16 (m, 2H), 7.14-6.88 (m, 1H), 5.44-4.95 (m, 1H), 4.94-4.72 (m, 1H), 4.71-3.99 (m, 3H), 3.98-3.62 (m, 1H), 3.61-3.02 (m, 3H), 2.99-2.88 (m, 1H), 2.85-2.61 (m, 2H), 2.11-1.69 (m, 6H), 1.66-1.45 (m, 1H), 1.31-0.88 (m, 11H). m/z: ES⁺ [M+H]⁺=523.20.

Peak #2: colourless solid (14 mg, 2% over 2 steps).

¹H NMR (400 MHz, DMSO-d₆, 300 K) δ 8.43-8.12 (m, 1H), 8.09-7.63 (m, 1H), 7.48-7.16 (m, 2H), 7.14-6.88 (m, 1H), 5.44-4.95 (m, 1H), 4.94-4.72 (m, 1H), 4.71-3.99 (m, 3H), 3.98-3.62 (m, 1H), 3.61-3.02 (m, 3H), 2.99-2.88 (m, 1H), 2.85-2.61 (m, 2H), 2.11-1.69 (m, 6H), 1.66-1.45 (m, 1H), 1.31-0.88 (m, 11H). m/z: ES⁺ [M+H]⁺=523.20.

### Example 1.066: 2-((4-((1S,5R)-2-(7-azabicyclo[2.2.1]heptane-1-carbonyl)-2,6-diazabicyclo[3.2.1]octan-6-yl)pyrimidin-5-yl)oxy)-N-ethyl-5-fluoro-N-isopropylbenzamide

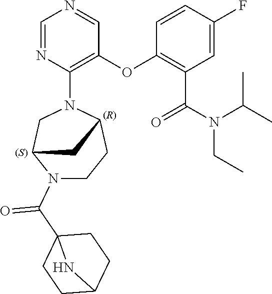

Off-white solid (24 mg, 35% over 2 steps).

¹H NMR (400 MHz, DMSO-d₆, 300 K) δ 8.40-8.25 (m, 1H), 7.85-7.70 (m, 1H), 7.35-7.20 (m, 2H), 7.10-6.95 (m, 1H), 6.04 (s, 1H), 5.20-5.00 (m, 1H), 4.85-4.60 (m, 1H), 4.40-4.15 (m, 1H), 3.83-3.75 (m, 2H), 3.75-3.60 (m, 2H), 3.53-3.10 (s, 2H), 2.00-1.86 (m, 1H), 1.86-1.75 (m, 1H), 1.75-1.65 (m, 3H), 1.65-1.50 (m, 5H), 1.40-1.30 (m, 2H), 1.17-0.96 (m, 10H). m/z: ES⁺ [M+H]⁺=537.25.

### Example 1.067: N-ethyl-5-fluoro-N-isopropyl-2-((4-((1S,5R)-2-((S)-3-(methylamino) butanoyl)-2,6-diazabicyclo[3.2.1]octan-6-yl)pyrimidin-5-yl)oxy)benzamide

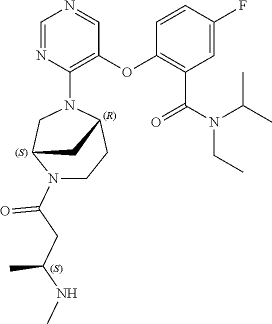

Colourless solid (56 mg, 55%).

¹H NMR (400 MHz, DMSO-d₆, 300 K) δ 8.40-8.26 (m, 1H), 7.85-7.55 (m, 1H), 7.45-7.25 (m, 2H), 7.01-6.85 (m, 1H), 5.40-4.77 (m, 1H), 4.76-4.37 (m, 1H), 4.36-3.60 (m, 3H), 3.56-3.40 (m, 1H), 3.25-3.05 (m, 3H), 2.87-2.77 (m, 1H), 2.77-2.66 (m, 1H), 2.56-2.34 (m, 2H), 2.17-2.10 (m, 3H), 1.98-1.85 (m, 2H), 1.83-1.75 (m, 2H), 0.96-0.58 (m, 12H). m/z: ES⁺ [M+H]⁺=513.35.

### Example 1.068: N-ethyl-5-fluoro-N-isopropyl-2-((4-((1S,5R)-2-((R)-3-(methylamino)butanoyl)-2,6-diazabicyclo[3.2.1]octan-6-yl)pyrimidin-5-yl)oxy)benzamide

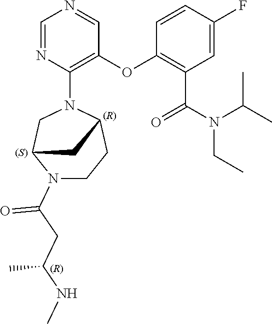

Off-white solid (62 mg, 82%).

¹H NMR (400 MHz, DMSO-d₆, 300 K) δ 8.34-8.31 (m, 1H), 7.87-7.73 (m, 1H), 7.27-7.24 (m, 2H), 7.13-6.91 (m, 1H), 5.16-5.15 (m, 1H), 4.76-4.56 (m, 1H), 4.49-4.08 (m, 1H), 3.79-3.73 (m, 3H), 3.65-3.45 (m, 1H), 3.23-3.21 (m, 2H), 2.87-2.59 (m, 2H), 2.49-2.42 (m, 1H), 2.23-2.22 (m, 3H), 2.10-1.96 (m, 1H), 1.87-1.81 (m, 2H), 1.79-1.52 (m, 2H), 1.21-0.90 (m, 12H). m/z: ES⁺ [M+H]⁺=513.30.

### Examples 1.069 and 1.070: 2-((4-((1S,5R)-2-((S)-3-(dimethylamino)butanoyl)-2,6-diazabicyclo[3.2.1]octan-6-yl)pyrimidin-5-yl)oxy)-N-ethyl-5-fluoro-N-isopropylbenzamide and 2-((4-((1S,5R)-2-((R)-3-(dimethylamino)butanoyl)-2,6-diazabicyclo[3.2.1]octan-6-yl)pyrimidin-5-yl)oxy)-N-ethyl-5-fluoro-N-isopropylbenzamide

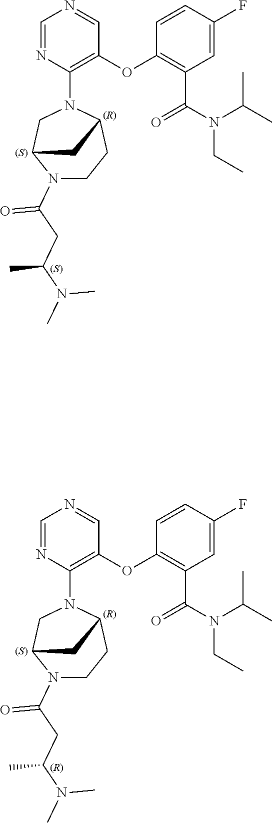

Separation by chiral HPLC gave two single stereoisomers:

Peak #1: colourless solid (23 mg, 17%).

¹H NMR (400 MHz, DMSO-d₆, 300 K) δ 8.33 (dd, J=10.2, 2.6 Hz, 1H), 7.88-7.73 (m, 1H), 7.27 (dddt, J=14.8, 8.8, 6.3, 3.1 Hz, 2H), 7.09-6.86 (m, 1H), 5.25-4.29 (m, 2H), 4.26-3.66 (m, 3H), 3.29-2.91 (m, 4H), 2.77-2.55 (m, 1H), 2.45 (dd, J=14.9, 4.7 Hz, 1H), 2.38-2.26 (m, 1H), 2.25-2.13 (m, 6H), 2.10-1.79 (m, 2H), 1.78-1.50 (m, 2H), 1.25-0.99 (m, 9H), 0.97-0.83 (m, 3H). m/z: ES⁺ [M+H]⁺=527.30.

Peak #2: colourless solid (25 mg, 18%).

¹H NMR (400 MHz, DMSO-d₆, 300 K) δ 8.33 (dd, J=10.6, 2.6 Hz, 1H), 7.87-7.74 (m, 1H), 7.35-7.17 (m, 2H), 7.09-6.89 (m, 1H), 5.14-4.35 (m, 2H), 4.23-3.67 (m, 3H), 3.64-3.37 (m, 2H), 3.28-2.95 (m, 3H), 2.73-2.54 (m, 1H), 2.47-2.22 (m, 1H), 2.16 (d, J=10.4 Hz, 6H), 2.07-1.77 (m, 2H), 1.76-1.49 (m, 2H), 1.26-1.00 (m, 9H), 0.91 (dt, J=13.5, 6.5 Hz, 3H). m/z: ES⁺ [M+H]⁺=527.30.

### Example 1.071: N-ethyl-5-fluoro-N-isopropyl-2-((4-((1S,5R)-2-(2-((R)-pyrrolidin-3-yl)acetyl)-2,6-diazabicyclo[3.2.1]octan-6-yl)pyrimidin-5-yl)oxy)benzamide

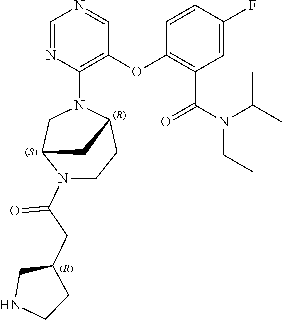

Colourless solid (30 mg, 51%).

m/z: ES⁺ [M+H]⁺=525.3.

### Example 1.072: N-ethyl-5-fluoro-N-isopropyl-2-((4-((1S,5R)-2-(2-((R)-1-methylpyrrolidin-3-yl)acetyl)-2,6-diazabicyclo[3.2.1]octan-6-yl)pyrimidin-5-yl)oxy)benzamide

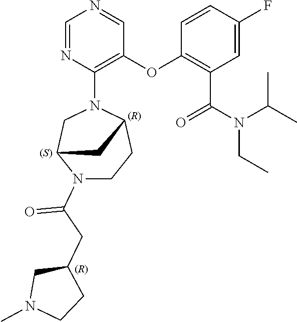

Yellow solid (12 mg, 10%).

¹H NMR (400 MHz, DMSO-d₆, 300 K) δ 8.43-8.22 (m, 1H), 7.95-7.65 (m, 1H), 7.41-6.74 (m, 3H), 5.22-4.25 (m, 2H), 4.18-3.39 (m, 5H), 3.28-3.02 (m, 2H), 2.66-2.03 (m, 9H), 1.99-1.75 (m, 4H), 1.74-1.30 (m, 3H), 1.29-0.98 (m, 9H). m/z: ES⁺ [M+H]⁺=527.30. m/z: ES⁺ [M+H]⁺=539.30

### Example 1.073: N-ethyl-5-fluoro-N-isopropyl-2-((4-((1S,5R)-2-(2-((R)-pyrrolidin-2-yl)acetyl)-2,6-diazabicyclo[3.2.1]octan-6-yl)pyrimidin-5-yl)oxy)benzamide

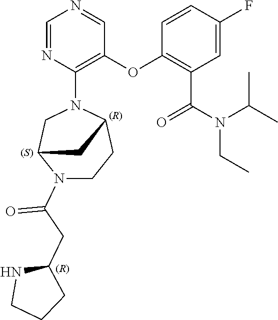

Yellow solid (15 mg, 26%).

m/z: ES⁺ [M+H]⁺=525.3.

### Examples 1.074 and 1.075: N-ethyl-5-fluoro-N-isopropyl-2-((4-((1S,5R)-2-((S)-2-((R)-pyrrolidin-2-yl)propanoyl)-2,6-diazabicyclo[3.2.1]octan-6-yl)pyrimidin-5-yl)oxy)benzamide and N-ethyl-5-fluoro-N-isopropyl-2-((4-((1S,5R)-2-((R)-2-((R)-pyrrolidin-2-yl)propanoyl)-2,6-diazabicyclo[3.2.1]octan-6-yl)pyrimidin-5-yl)oxy)benzamide

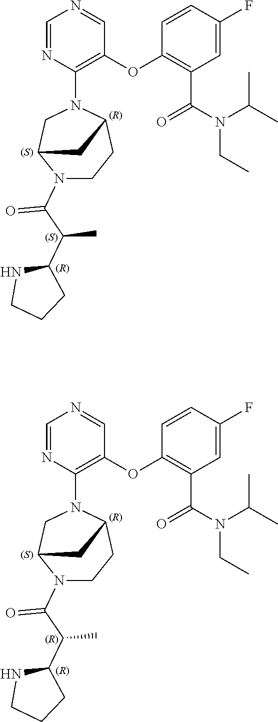

Separation by chiral HPLC gave two single stereoisomers:

Peak #1: colourless solid, (6 mg, 12% over 2 steps).

¹H NMR (400 MHz, Methanol-d₄) δ 8.35-8.28 (m, 1H), 7.90-7.78 (m, 1H), 7.23-7.14 (m, 2H), 6.97-6.80 (m, 1H), 5.40-4.90 (m, 1H), 4.90-4.80 (m, 1H), 4.60-4.40 (m, 1H), 4.12-3.60 (m, 4H), 3.60-3.40 (m, 1H), 3.40-3.30 (m, 2H), 3.10-2.90 (m, 2H), 2.83-2.70 (m, 1H), 2.20-1.90 (m, 3H), 1.80-1.60 (m, 4H), 1.60-1.40 (m, 1H), 1.40-1.30 (m, 2H), 1.30-1.20 (m, 6H), 1.20-1.10 (m, 5H). m/z: ES⁺ [M+H]⁺=539.30.

Peak #2: colourless solid, (22 mg, 44% over 2 steps).

¹H NMR (400 MHz, Methanol-d₄) δ 8.35-8.28 (m, 1H), 7.90-7.78 (m, 1H), 7.23-7.14 (m, 2H), 6.97-6.80 (m, 1H), 5.40-4.90 (m, 1H), 4.90-4.80 (m, 1H), 4.60-4.40 (m, 1H), 4.12-3.60 (m, 4H), 3.60-3.40 (m, 1H), 3.40-3.30 (m, 2H), 3.10-2.90 (m, 2H), 2.83-2.70 (m, 1H), 2.20-1.90 (m, 3H), 1.80-1.60 (m, 4H), 1.60-1.40 (m, 1H), 1.40-1.30 (m, 2H), 1.30-1.20 (m, 6H), 1.20-1.10 (m, 5H). m/z: ES⁺ [M+H]⁺=539.30.

### Examples 1.076 and 1.077: N-ethyl-5-fluoro-N-isopropyl-2-(4-((1S,5R)-2-((R)-2-((R)-pyrrolidin-3-yl)propanoyl)-2,6-diaza-bicyclo[3.2.1]octan-6-yl)pyrimidin-5-yloxy)benzamide and N-ethyl-5-fluoro-N-isopropyl-2-(4-((1S,5R)-2-((S)-2-((R)-pyrrolidin-3-yl)propanoyl)-2,6-diaza-bicyclo[3.2.1]octan-6-yl)pyrimidin-5-yloxy)benzamide

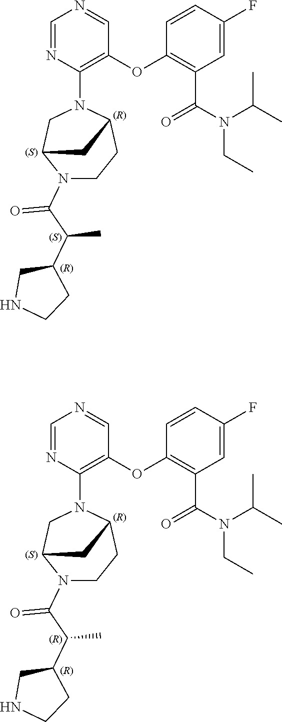

Separation by chiral HPLC gave two single stereoisomers:

Peak #1: colourless solid, TFA salt (9 mg, 6% over 2 steps).

¹H NMR (400 MHz, DMSO-d₆, 300 K) δ 8.35-8.31 (m, 1H), 7.95-7.82 (m, 1H), 7.21-7.11 (m, 2H), 7.10-6.92 (m, 1H), 5.21-4.05 (m, 3H), 3.89-3.70 (m, 3H), 3.41-3.30 (m, 2H), 3.20-3.05 (m, 3H), 3.01-2.85 (m, 2H), 2.75-2.60 (m, 2H), 2.35-2.05 (m, 1H), 1.95-1.75 (m, 2H), 1.71-1.50 (m, 3H), 1.41-0.87 (m, 13H). m/z: ES⁺ [M+H]⁺=539.30.

Peak #2: colourless solid, TFA salt (23 mg, 15% over 2 steps).

¹H NMR (400 MHz, DMSO-d₆, 300 K) δ 8.36-8.30 (m, 1H), 7.97-7.81 (m, 1H), 7.22-7.10 (m, 2H), 7.11-6.93 (m, 1H), 5.25-4.01 (m, 3H), 3.98-3.71 (m, 3H), 3.42-3.10 (m, 3H), 3.01-2.65 (m, 3H), 2.60-2.50 (m, 1H), 2.25-2.15 (m, 1H), 2.10-2.01 (m, 1H), 1.98-1.75 (m, 3H), 1.61-1.40 (m, 2H), 1.35-1.31 (m, 1H), 1.25-0.85 (m, 13H). m/z: ES⁺ [M+H]⁺=539.30.

### Example 1.078: N-ethyl-5-fluoro-N-isopropyl-2-(4-((1S,5R)-2-(2-((R)-3-methylpyrrolidin-3-yl)acetyl)-2,6-diaza-bicyclo[3.2.1]octan-6-yl)pyrimidin-5-yloxy) benzamide and N-ethyl-5-fluoro-N-isopropyl-2-(4-((1S,5R)-2-(2-((S)-3-methylpyrrolidin-3-yl)acetyl)-2,6-diaza-bicyclo[3.2.1]octan-6-yl)pyrimidin-5-yloxy) benzamide

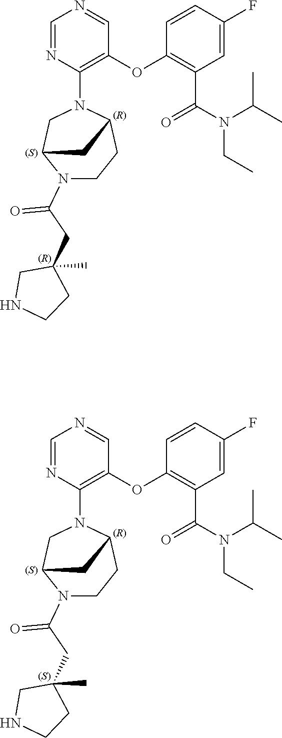

The mixture of 2 diastereoisomers was not separated, light yellow oil (7 mg, 11% over 2 steps).

¹H NMR (400 MHz, DMSO-d₆, 300 K) δ 8.37-8.27 (m, 1H), 7.85-7.73 (m, 1H), 7.29-7.25 (m, 2H), 7.22-6.96 (m, 1H), 5.19-5.83 (m, 1H), 4.74-4.69 (m, 1H), 4.46-4.09 (m, 1H), 3.80-3.77 (m, 3H), 3.48-3.39 (m, 1H), 3.18-3.12 (m, 4H), 2.82-2.72 (m, 1H), 2.70-2.68 (m, 1H), 2.55-2.50 (m, 1H), 2.45-2.24 (m, 2H), 1.95-1.77 (m, 2H), 1.73-1.37 (m, 4H), 1.08-0.98 (m, 12H). m/z: ES⁺ [M+H]⁺=539.20.

### Examples 1.079 and 1.080: N-ethyl-5-fluoro-N-isopropyl-2-((4-((1S,5R)-2-((3aS,6aS)-octahydrocyclopenta[c]pyrrole-3a-carbonyl)-2,6-diazabicyclo[3.2.1]octan-6-yl)pyrimidin-5-yl)oxy)benzamide and N-ethyl-5-fluoro-N-isopropyl-2-((4-((1S,5R)-2-((3aR,6aR)-octahydrocyclopenta[c]pyrrole-3a-carbonyl)-2,6-diazabicyclo[3.2.1]octan-6-yl)pyrimidin-5-yl)oxy)benzamide

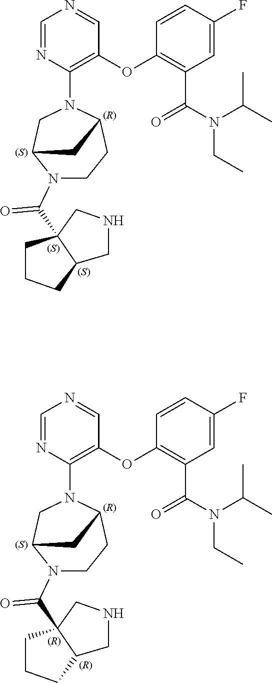

Separation by chiral HPLC gave two single stereoisomers:

Peak #1: colourless solid (18 mg, 16% over 2 steps).

¹H NMR (400 MHz, DMSO-d₆, 300 K) δ 8.44-8.25 (m, 1H), 7.91-7.70 (m, 1H), 7.40-7.15 (m, 2H), 7.12-6.86 (m, 1H), 5.26-4.96 (m, 1H), 4.95-4.79 (m, 1H), 4.78-4.47 (m, 1H), 4.46-4.01 (m, 1H), 4.00-3.71 (m, 3H), 3.25-3.03 (m, 3H), 3.02-2.92 (m, 2H), 2.91-2.76 (m, 1H), 2.55-2.40 (m, 2H), 2.06-1.75 (m, 5H), 1.73-1.54 (m, 4H), 1.51-1.26 (m, 1H), 1.24-0.86 (m, 9H). m/z: ES⁺ [M+H]⁺=551.10.

Peak #2: colourless solid, (23 mg, 20% over 2 steps).

¹H NMR (400 MHz, DMSO-d₆, 300 K) δ 8.44-8.25 (m, 1H), 7.91-7.70 (m, 1H), 7.40-7.15 (m, 2H), 7.12-6.86 (m, 1H), 5.26-4.96 (m, 1H), 4.95-4.79 (m, 1H), 4.78-4.47 (m, 1H), 4.46-4.01 (m, 1H), 4.00-3.71 (m, 3H), 3.25-3.03 (m, 3H), 3.02-2.92 (m, 2H), 2.91-2.76 (m, 1H), 2.55-2.40 (m, 2H), 2.06-1.75 (m, 5H), 1.73-1.54 (m, 4H), 1.51-1.26 (m, 1H), 1.24-0.86 (m, 9H). m/z: ES⁺ [M+H]⁺=551.10.

### Example 1.081: 2-((4-((1S,5R)-2-((1r,3S)-3-aminocyclobutane-1-carbonyl)-2,6-diazabicyclo[3.2.1]octan-6-yl)pyrimidin-5-yl)oxy)-N-ethyl-5-fluoro-N-isopropylbenzamide

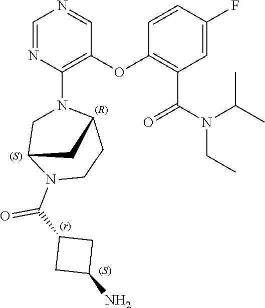

Colourless solid (24 mg, 42%).

m/z: ES⁺ [M+H]⁺=511.3.

### Example 1.082: 2-((4-((1S,5R)-2-((1s,3R)-3-aminocyclobutane-1-carbonyl)-2,6-diazabicyclo[3.2.1]octan-6-yl)pyrimidin-5-yl)oxy)-N-ethyl-5-fluoro-N-isopropylbenzamide

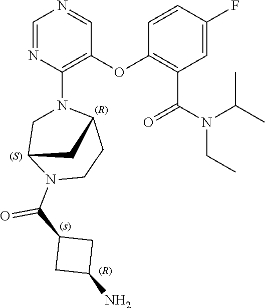

Colourless solid (29 mg, 51%).

m/z: ES⁺ [M+H]⁺=511.3.

### Example 1.083: 2-((4-((1S,5R)-2-(4-aminobutanoyl)-2,6-diazabicyclo[3.2.1]octan-6-yl)pyrimidin-5-yl)oxy)-N-ethyl-5-fluoro-N-isopropylbenzamide

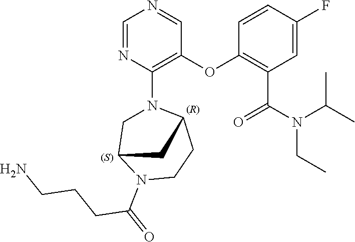

Colourless oil (13 mg, 23%).

m/z: ES⁺ [M+H]⁺=499.3.

### Example 1.084: 2-((4-((1S,5R)-2-(2-(azetidin-3-yl)acetyl)-2,6-diazabicyclo[3.2.1]octan-6-yl)pyrimidin-5-yl)oxy)-N-ethyl-5-fluoro-N-isopropylbenzamide

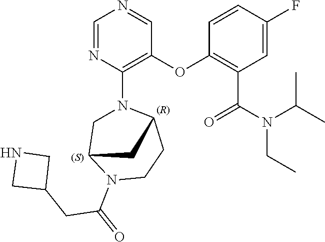

Colourless solid (26 mg, 46%).

m/z: ES⁺ [M+H]⁺=511.3.

### Examples 1.085 and 1.086: 2-((4-((1S,5R)-2-((S)-2-(azetidin-3-yl)propanoyl)-2,6-diazabicyclo[3.2.1]octan-6-yl)pyrimidin-5-yl)oxy)-N-ethyl-5-fluoro-N-isopropylbenzamide and 2-((4-((1S,5R)-2-((R)-2-(azetidin-3-yl)propanoyl)-2,6-diazabicyclo[3.2.1]octan-6-yl)pyrimidin-5-yl)oxy)-N-ethyl-5-fluoro-N-isopropylbenzamide

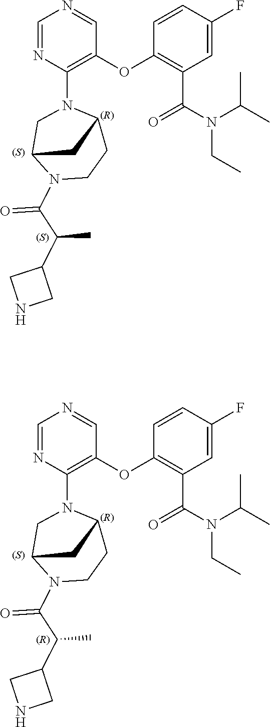

Separation by chiral HPLC gave two single stereoisomers:

Peak #1: colourless solid (28 mg, 16% over 2 steps).

¹H NMR (400 MHz, DMSO-d₆, 300 K) δ 8.43-8.22 (m, 1H), 7.95-7.65 (m, 1H), 7.41-6.74 (m, 3H), 4.92-4.25 (m, 3H), 3.91-3.49 (m, 5H), 3.28-2.82 (m, 6H), 2.26-1.63 (m, 5H), 1.32-0.98 (m, 13H). m/z: ES⁺ [M+H]⁺=525.20.

Peak #2: colourless solid (40 mg, 23% over 2 steps).

¹H NMR (400 MHz, DMSO-d₆, 300 K) δ 8.43-8.22 (m, 1H), 7.95-7.65 (m, 1H), 7.41-6.74 (m, 3H), 4.92-4.25 (m, 3H), 3.91-3.49 (m, 5H), 3.28-2.82 (m, 6H), 2.26-1.63 (m, 5H), 1.32-0.98 (m, 13H). m/z: ES⁺ [M+H]⁺=525.20.

### Example 1.087: N-ethyl-5-fluoro-2-((4-((1S,5R)-2-(3-fluoroazetidine-3-carbonyl)-2,6-diazabicyclo[3.2.1]octan-6-yl)pyrimidin-5-yl)oxy)-N-isopropylbenzamide

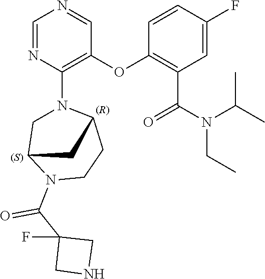

Colourless solid (25 mg, 44%).

m/z: ES⁺ [M+H]⁺=515.3.

### Example 1.088: 2-((4-((1S,5R)-2-(N-acetyl-N-methylglycyl)-2,6-diazabicyclo[3.2.1]octan-6-yl)pyrimidin-5-yl)oxy)-N-ethyl-5-fluoro-N-isopropylbenzamide

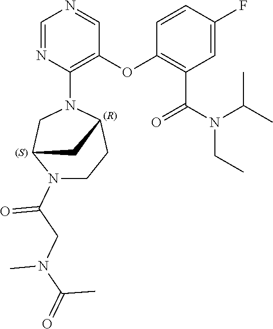

Colourless solid (20 mg, 57%).

m/z: ES⁺ [M+H]⁺=527.3.

### Example 1.089: N-ethyl-5-fluoro-N-isopropyl-2-((4-((1S,5R)-2-((R)-piperidine-3-carbonyl)-2,6-diazabicyclo[3.2.1]octan-6-yl)pyrimidin-5-yl)oxy)benzamide

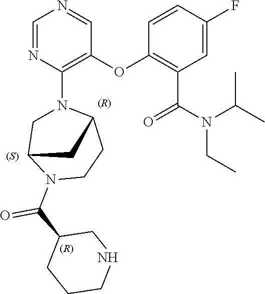

Colourless solid (25 mg, 43%).

m/z: ES⁺ [M+H]⁺=525.3.

### Example 1.090: N-ethyl-5-fluoro-N-isopropyl-2-((4-((1S,5R)-2-((S)-piperidine-3-carbonyl)-2,6-diazabicyclo[3.2.1]octan-6-yl)pyrimidin-5-yl)oxy)benzamide

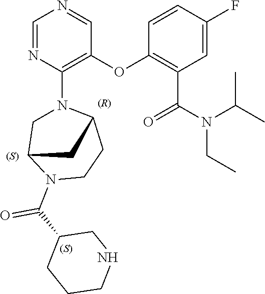

Colourless solid (25 mg, 43%).

m/z: ES⁺ [M+H]⁺=525.3.

### Example 1.091: N-ethyl-5-fluoro-N-isopropyl-2-((4-((1S,5R)-2-(piperidine-4-carbonyl)-2,6-diazabicyclo[3.2.1]octan-6-yl)pyrimidin-5-yl)oxy)benzamide

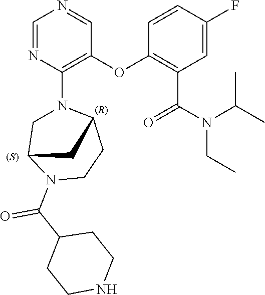

Colourless solid (11 mg, 19%).

m/z: ES⁺ [M+H]⁺=525.3.

### Example 1.092: 2-((4-((1S,5R)-2-((R)-3-aminobutanoyl)-2,6-diazabicyclo[3.2.1]octan-6-yl)pyrimidin-5-yl)oxy)-N-ethyl-5-fluoro-N-isopropylbenzamide

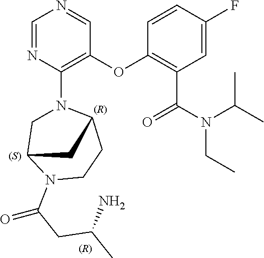

Colourless solid (12 mg, 22%).

m/z: ES⁺ [M+H]⁺=499.3.

### Example 1.093: N-ethyl-5-fluoro-N-isopropyl-2-((4-((1S,5R)-2-(2-(piperazin-1-yl)acetyl)-2,6-diazabicyclo[3.2.1]octan-6-yl)pyrimidin-5-yl)oxy)benzamide

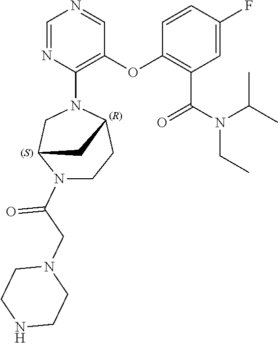

Yellow solid (19 mg, 32%).

m/z: ES⁺ [M+H]⁺=540.3.

### Example 1.094: N-ethyl-5-fluoro-N-isopropyl-2-((4-((1S,5R)-2-(2-(4-methylpiperazin-1-yl)acetyl)-2,6-diazabicyclo[3.2.1]octan-6-yl)pyrimidin-5-yl)oxy)benzamide

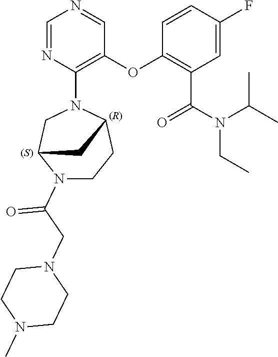

Colourless solid (28 mg, 76%).

m/z: ES⁺ [M+H]⁺=554.4.

### Example 1.095: 2-((4-((1S,5R)-2-((S)-3-amino-2-methylpropanoyl)-2,6-diazabicyclo[3.2.1]octan-6-yl)pyrimidin-5-yl)oxy)-N-ethyl-5-fluoro-N-isopropylbenzamide

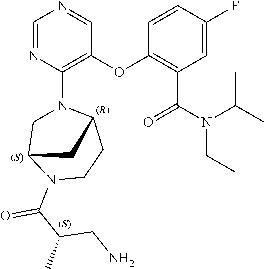

Colourless oil (30 mg, 54%).

m/z: ES⁺ [M+H]⁺=499.3.

### Example 1.096: N-ethyl-5-fluoro-N-isopropyl-2-((4-((1S,5R)-2-(3-methoxypropanoyl)-2,6-diazabicyclo[3.2.1]octan-6-yl)pyrimidin-5-yl)oxy)benzamide

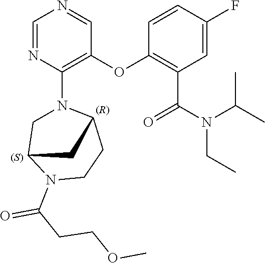

Colourless solid (23 mg, 69%).

m/z: ES⁺ [M+H]⁺=500.3.

### Example 1.097: N-ethyl-5-fluoro-N-isopropyl-2-((4-((1S,5R)-2-(O-methyl-L-seryl)-2,6-diazabicyclo[3.2.1]octan-6-yl)pyrimidin-5-yl)oxy)benzamide

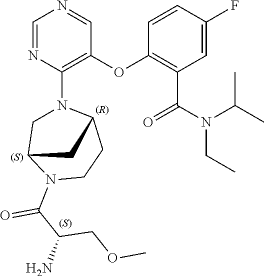

Colourless oil (25 mg, 44%).

m/z: ES⁺ [M+H]⁺=515.3.

### Example 1.098: 2-((4-((1S,5R)-2-((S)-4-aminopentanoyl)-2,6-diazabicyclo[3.2.1]octan-6-yl)pyrimidin-5-yl)oxy)-N-ethyl-5-fluoro-N-isopropylbenzamide

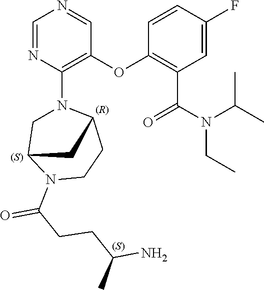

Colourless oil (34 mg, 59%).

m/z: ES⁺ [M+H]⁺=513.3.

### Example 1.099: N-ethyl-5-fluoro-N-isopropyl-2-((4-((1S,5R)-2-(3-(methylamino)propanoyl)-2,6-diazabicyclo[3.2.1]octan-6-yl)pyrimidin-5-yl)oxy)benzamide

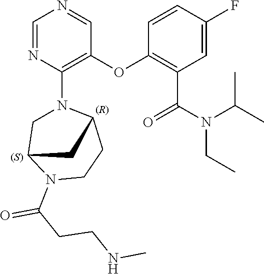

Colourless solid (23 mg, 41%).

m/z: ES⁺ [M+H]⁺=499.3.

### Example 1.100: 2-((4-((1S,5R)-2-((S)-3-aminobutanoyl)-2,6-diazabicyclo[3.2.1]octan-6-yl)pyrimidin-5-yl)oxy)-N-ethyl-5-fluoro-N-isopropylbenzamide

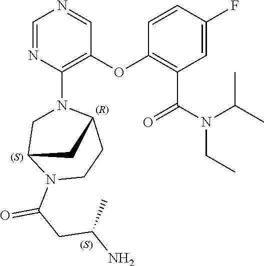

Colourless oil (12 mg, 22%).

m/z: ES⁺ [M+H]⁺=499.3.

### Example 1.101: N-ethyl-5-fluoro-N-isopropyl-2-((4-((1S,5R)-2-(O-methyl-D-seryl)-2,6-diazabicyclo[3.2.1]octan-6-yl)pyrimidin-5-yl)oxy)benzamide

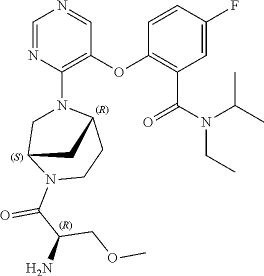

Colourless oil (6 mg, 10%).

m/z: ES⁺ [M+H]⁺=515.3.

### Example 1.102: 2-((4-((1S,5R)-2-(azetidine-3-carbonyl)-2,6-diazabicyclo[3.2.1]octan-6-yl)pyrimidin-5-yl)oxy)-N-ethyl-5-fluoro-N-isopropylbenzamide

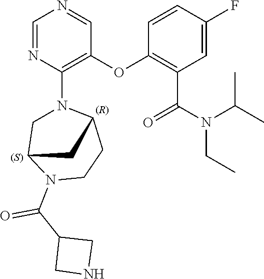

Colourless oil (12 mg, 24%).

m/z: ES⁺ [M+H]⁺=497.2.

### Example 1.103: N-ethyl-5-fluoro-N-isopropyl-2-((4-((1S,5R)-2-((1R,3R,5R)-3-methyl-2-azabicyclo[3.1.0]hexane-3-carbonyl)-2,6-diazabicyclo[3.2.1]octan-6-yl)pyrimidin-5-yl)oxy)benzamide and N-ethyl-5-fluoro-N-isopropyl-2-((4-((1S,5R)-2-((1R,3S,5R)-3-methyl-2-azabicyclo[3.1.0]hexane-3-carbonyl)-2,6-diazabicyclo[3.2.1]octan-6-yl)pyrimidin-5-yl)oxy)benzamide

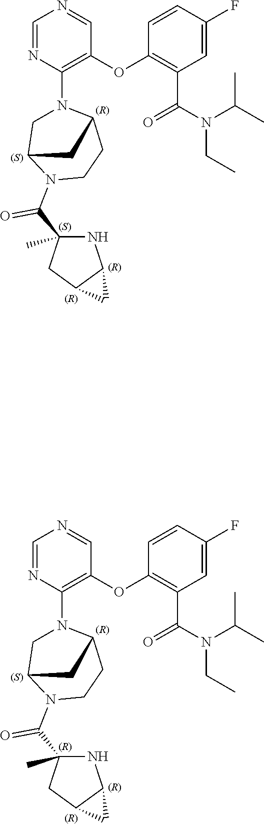

Colourless solid (6 mg, 22% over 2 steps).

¹H NMR (400 MHz, DMSO-d₆, 298 K) δ 8.36-8.30 (m, 1H), 7.97-7.81 (m, 1H), 7.22-7.10 (m, 2H), 7.11-6.93 (m, 1H), 5.51-5.01 (m, 1H), 4.98-4.25 (m, 1H), 4.22-4.10 (m, 1H), 4.01-3.55 (m, 3H), 3.44-3.33 (m, 1H), 3.30-3.15 (m, 2H), 3.10-2.80 (m, 1H), 2.70-2.60 (m, 1H), 2.21-2.14 (m, 1H), 1.95-1.75 (m, 3H), 1.74-1.45 (m, 2H), 1.41-1.31 (m, 4H), 1.30-1.21 (m, 2H), 1.20-0.99 (m, 8H), 0.53-0.30 (m, 1H), 0.10-0.05 (m, 1H).

m/z: ES⁺ [M+H]⁺=537.25.

### Example 1.104: N-ethyl-5-fluoro-N-isopropyl-2-((4-((1S,5R)-2-((1S,3R,5S)-3-methyl-2-azabicyclo[3.1.0]hexane-3-carbonyl)-2,6-diazabicyclo[3.2.1]octan-6-yl)pyrimidin-5-yl)oxy)benzamide and N-ethyl-5-fluoro-N-isopropyl-2-((4-((1S,5R)-2-((1S,3S,5S)-3-methyl-2-azabicyclo[3.1.0]hexane-3-carbonyl)-2,6-diazabicyclo[3.2.1]octan-6-yl)pyrimidin-5-yl)oxy)benzamide

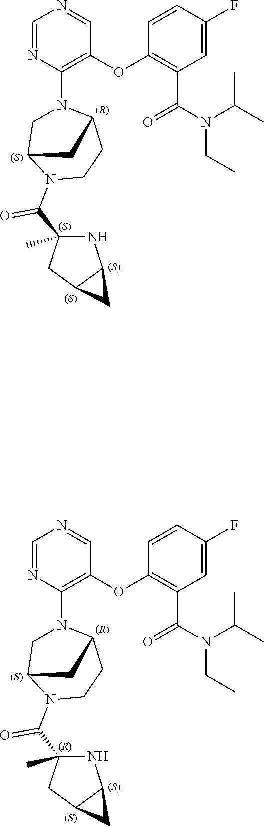

Off-white solid (10 mg, 22% over 2 steps).

¹H NMR (400 MHz, DMSO-d₆, 300 K) δ 8.44-8.27 (m, 1H), 7.95-7.68 (m, 1H), 7.50-7.19 (m, 2H), 7.19-6.74 (m, 1H), 5.50-4.98 (m, 1H), 4.98-4.54 (m, 1H), 4.54-4.01 (m, 1H), 4.01-3.55 (m, 3H), 3.50-2.95 (m, 4H), 2.66-2.57 (m, 1H), 2.51-2.34 (m, 2H), 2.07-1.45 (m, 5H), 1.45-0.88 (m, 12H), 0.54-0.31 (m, 1H), 0.10-0.09 (m, 1H) m/z: ES⁺ [M+H]⁺=537.20.

### Examples 1.105 and 1.106: 2-((4-((1S,5R)-2-((S)-2-azabicyclo[2.1.1]hexane-3-carbonyl)-2,6-diazabicyclo[3.2.1]octan-6-yl)pyrimidin-5-yl)oxy)-N-ethyl-5-fluoro-N-isopropylbenzamide and 2-((4-((1S,5R)-2-((R)-2-azabicyclo[2.1.1]hexane-3-carbonyl)-2,6-diazabicyclo[3.2.1]octan-6-yl)pyrimidin-5-yl)oxy)-N-ethyl-5-fluoro-N-isopropylbenzamide

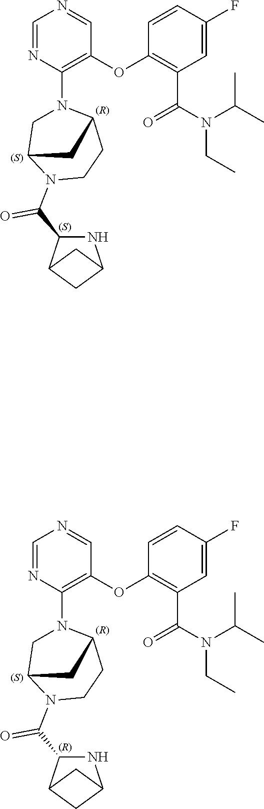

Separation by chiral HPLC gave two single stereoisomers:

Peak #1: Brown semi-solid (28 mg, 20% over 2 steps).

1H NMR (400 MHz, DMSO-d₆, 300 K) δ 8.40-8.25 (m, 1H), 7.89-7.68 (m, 1H), 7.33-7.18 (m, 2H), 7.13-6.93 (m, 1H), 5.26-5.03 (m, 1H), 4.83-4.61 (m, 1H), 4.45-3.92 (m, 2H), 3.90-3.55 (m, 4H), 3.31-3.04 (m, 2H), 2.99-2.74 (m, 1H), 2.12-1.84 (m, 2H), 1.84-1.63 (m, 3H), 2.12-1.89 (m, 1H), 1.43-1.25 (m, 1H), 1.25-1.14 (m, 2H), 1.14-0.88 (m, 10H). m/z: ES⁺ [M+H]⁺=523.30.

Peak #2: Colourless solid (21 mg, 14% over 2 steps).

1H NMR (400 MHz, DMSO-d₆, 300 K) δ 8.40-8.25 (m, 1H), 7.89-7.68 (m, 1H), 7.33-7.18 (m, 2H), 7.13-6.93 (m, 1H), 5.26-5.03 (m, 1H), 4.83-4.61 (m, 1H), 4.45-3.92 (m, 2H), 3.90-3.55 (m, 4H), 3.31-3.04 (m, 2H), 2.99-2.74 (m, 1H), 2.12-1.84 (m, 2H), 1.84-1.63 (m, 3H), 2.12-1.89 (m, 1H), 1.43-1.25 (m, 1H), 1.25-1.14 (m, 2H), 1.14-0.88 (m, 10H). m/z: ES⁺ [M+H]⁺=523.35.

### Example 1.107: N-ethyl-5-fluoro-2-((4-((1S,5R)-2-(hexahydro-1H-pyrrolizine-7a-carbonyl)-2,6-diazabicyclo[3.2.1]octan-6-yl)pyrimidin-5-yl)oxy)-N-isopropylbenzamide

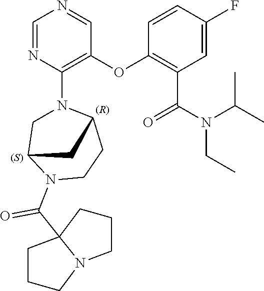

Colourless solid, trifluoroacetic acid salt (54 mg, 48%).

¹H NMR (400 MHz, DMSO-d₆, 300 K) δ 9.89 (br s, 1H), 8.62 (s, 1H), 8.10-8.00 (m, 1H), 7.45-7.15 (m, 3H), 5.18-4.85 (m, 3H), 4.05-3.84 (m, 3H), 3.84-3.61 (m, 2H), 3.55-3.40 (m, 4H), 3.28-3.09 (m, 3H), 3.05-2.90 (m, 1H), 2.46-2.23 (m, 3H), 2.25-2.05 (m, 2H), 2.04-1.67 (m, 6H), 1.22-0.93 (m, 9H). m/z: ES⁺ [M+H]⁺=551.3.

### Example 1.108: 2-((4-((1S,5R)-2-((R)-azepane-2-carbonyl)-2,6-diazabicyclo[3.2.1]octan-6-yl)pyrimidin-5-yl)oxy)-N-ethyl-5-fluoro-N-isopropylbenzamide

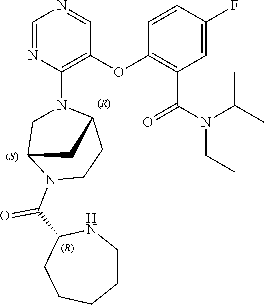

Off-white solid (64 mg, 53%).

¹H NMR (400 MHz, DMSO-d₆, 300 K) δ 8.38-8.30 (m, 1H), 8.26-8.10 (m, 1H), 7.90-7.80 (m, 1H), 7.35-7.18 (m, 2H), 7.10-6.90 (m, 1H), 5.10-5.06 (m, 1H), 4.90-4.80 (m, 1H), 4.70-4.80 (m, 1H), 4.60-4.70 (m, 1H), 4.50-4.40 (m, 1H), 4.30-4.40 (m, 1H), 4.20-4.00 (m, 1H), 4.00-3.90 (m, 1H), 3.90-3.80 (m, 1H), 3.80-3.70 (m, 2H), 3.40-3.30 (m, 1H), 3.00-2.90 (m, 1H), 2.90-2.70 (m, 1H), 2.00-1.76 (m, 3H), 1.70-1.40 (m, 8H), 1.40-0.90 (m, 9H). m/z: ES⁺ [M+H]⁺=539.25.

### Example 1.109: 2-((4-((1S,5R)-2-((S)-azepane-2-carbonyl)-2,6-diazabicyclo[3.2.1]octan-6-yl)pyrimidin-5-yl)oxy)-N-ethyl-5-fluoro-N-isopropylbenzamide

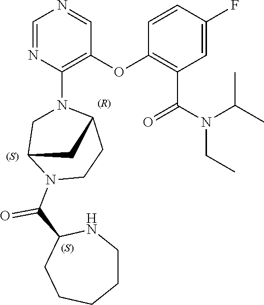

Yellow Solid (64 mg, 49% over 2 steps).

¹H NMR (400 MHz, DMSO-d₆, 300 K) δ 8.45-8.06 (m, 1H), 7.97-7.65 (m, 1H), 7.38-7.18 (m, 2H), 7.10-6.60 (m, 1H), 5.21-5.02 (m, 1H), 4.97-4.24 (m, 2H), 4.13-3.91 (m, 1H), 3.90-3.67 (m, 3H), 3.65-3.44 (m, 2H), 3.26-3.15 (m, 1H), 3.14-3.02 (m, 1H), 2.98-2.78 (m, 1H), 2.68-2.55 (m, 1H), 2.01-1.85 (m, 2H), 1.85-1.35 (m, 10H), 1.25-0.98 (m, 10H). m/z: ES⁺ [M+H]⁺=539.30.

### Example 1.110 and 1.111: N-ethyl-5-fluoro-N-isopropyl-2-((4-((1S,5R)-2-((R)-6-methyl-5-azaspiro[2.4]heptane-6-carbonyl)-2,6-diazabicyclo[3.2.1]octan-6-yl)pyrimidin-5-yl)oxy)benzamide and N-ethyl-5-fluoro-N-isopropyl-2-((4-((1S,5R)-2—((S)-6-methyl-5-azaspiro[2.4]heptane-6-carbonyl)-2,6-diazabicyclo[3.2.1]octan-6-yl)pyrimidin-5-yl)oxy)benzamide

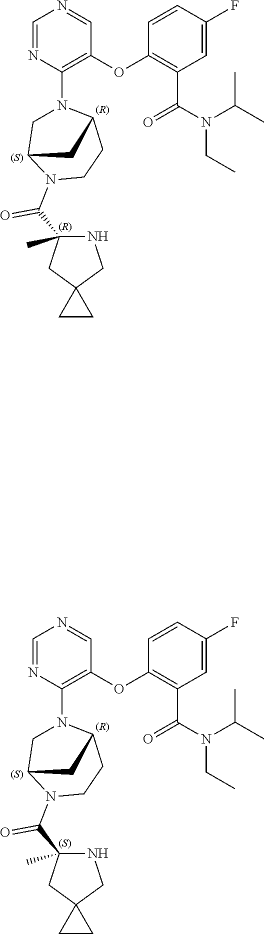

Separation by chiral HPLC gave two single stereoisomers:

Peak #1: Colourless solid (28 mg, 4% over 2 steps).

¹H NMR (400 MHz, DMSO-d₆, 300 K) δ 8.53-8.21 (m, 1H), 8.02-7.61 (m, 1H), 7.45-7.13 (m, 2H), 7.10-6.78 (m, 1H), 5.70-5.00 (m, 1H), 4.95-4.50 (m, 1H), 4.49-4.10 (m, 1H), 3.93-3.51 (m, 3H), 3.43-3.37 (m, 1H), 3.27-3.07 (m, 2H), 2.80-2.52 (m, 3H), 2.30-2.08 (m, 1H), 2.01-1.45 (m, 5H), 1.40-1.28 (m, 3H), 1.23-0.99 (m, 9H), 0.65-0.25 (m, 4H). m/z: ES⁺ [M+H]⁺=551.30.

Peak #2: Colourless solid (33 mg, 5% over 2 steps).

¹H NMR (400 MHz, DMSO-d₆, 300 K) δ 8.47-8.19 (m, 1H), 7.98-7.65 (m, 1H), 7.42-7.18 (m, 2H), 7.14-6.80 (m, 1H), 5.65-5.03 (m, 1H), 4.95-4.56 (m, 1H), 4.55-4.10 (m, 1H), 4.00-3.51 (m, 3H), 3.42-3.38 (m, 1H), 3.29-3.00 (m, 2H), 2.78-2.61 (m, 2H), 2.30-2.12 (m, 1H), 2.00-1.83 (m, 2H), 1.83-1.45 (m, 4H), 1.41-1.23 (m, 3H), 1.25-0.89 (m, 9H), 0.60-0.29 (m, 4H). m/z: ES⁺ [M+H]⁺=551.25.

### Example 1.112: N-ethyl-5-fluoro-N-isopropyl-2-((4-((1S,5R)-2-((1R,2R,5S)-2-methyl-3-azabicyclo[3.1.0]hexane-2-carbonyl)-2,6-diazabicyclo[3.2.1]octan-6-yl)pyrimidin-5-yl)oxy)benzamide and N-ethyl-5-fluoro-N-isopropyl-2-((4-((1S,5R)-2-((1R,2S,5S)-2-methyl-3-azabicyclo[3.1.0]hexane-2-carbonyl)-2,6-diazabicyclo[3.2.1]octan-6-yl)pyrimidin-5-yl)oxy)benzamide

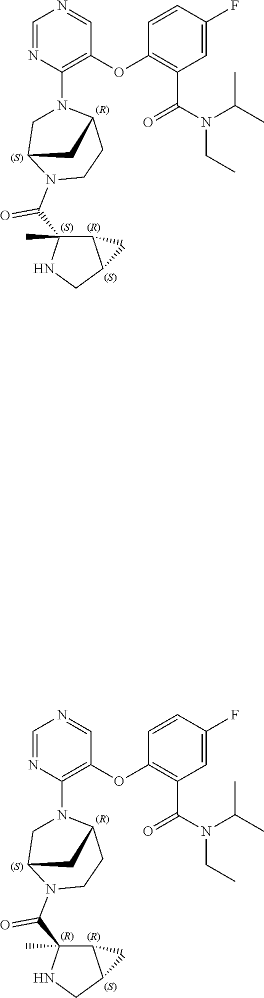

Colourless solid (7 mg, 16% over 2 steps)

¹H NMR (400 MHz, DMSO-d₆, 300 K) δ 8.45-8.20 (m, 1H), 7.91-7.69 (m, 1H), 7.41-7.13 (m, 2H), 7.10-6.81 (m, 1H), 6.26-5.82 (m, 1H), 5.41-4.49 (m, 2H), 4.48-4.01 (m, 1H), 3.95-3.48 (m, 3H), 3.32-3.28 (m, 1H), 3.29-3.05 (m, 2H), 2.79-2.61 (m, 3H), 2.10-1.96 (m, 1H), 1.95-1.75 (m, 2H), 1.70-1.50 (m, 2H), 1.30-1.15 (m, 6H), 1.14-0.96 (m, 8H), 0.41-0.19 (m, 2H). m/z: ES⁺ [M+H]⁺=537.25.

### Example 1.113: N-ethyl-5-fluoro-N-isopropyl-2-((4-((1S,5R)-2-((1S,2R,5R)-2-methyl-3-azabicyclo[3.1.0]hexane-2-carbonyl)-2,6-diazabicyclo[3.2.1]octan-6-yl)pyrimidin-5-yl)oxy)benzamide and N-ethyl-5-fluoro-N-isopropyl-2-((4-((1S,5R)-2-((1S,2S,5R)-2-methyl-3-azabicyclo[3.1.0]hexane-2-carbonyl)-2,6-diazabicyclo[3.2.1]octan-6-yl)pyrimidin-5-yl)oxy)benzamide

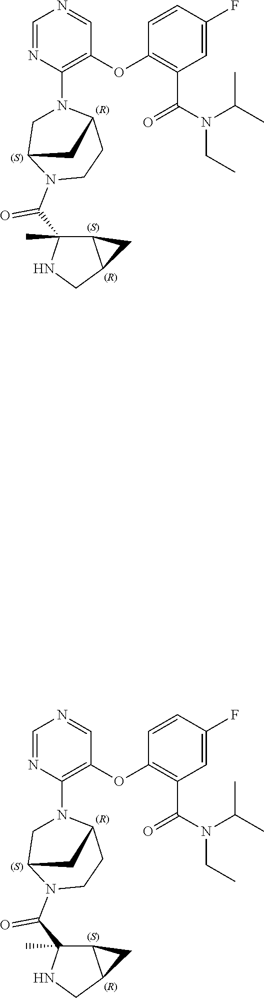

Off-white solid (4 mg, 2% over 2 steps).

¹H NMR (400 MHz, DMSO-d₆, 300 K) δ 8.50-8.12 (m, 1H), 7.95-7.62 (m, 1H), 7.40-7.12 (m, 2H), 7.10-6.70 (m, 1H), 6.50-5.65 (m, 1H), 5.55-4.95 (m, 1H), 4.95-4.55 (m, 1H), 4.50-4.05 (m, 1H), 3.95-3.55 (m, 3H), 3.50-3.35 (m, 1H), 3.30-2.95 (m, 2H), 2.80-2.70 (m, 1H), 2.50-2.60 (m, 1H), 2.05-1.95 (m, 1H), 1.87-1.65 (m, 2H), 1.65-1.50 (m, 2H), 1.35-1.20 (m, 4H), 1.20-1.15 (m, 1H), 1.20-0.92 (m, 9H), 0.40-0.15 (m, 2H). m/z: ES⁺ [M+H]⁺=537.30.

### Example 1.114: 2-((4-((1S,5R)-2-(2-(1H-imidazol-1-yl)acetyl)-2,6-diazabicyclo[3.2.1]octan-6-yl)pyrimidin-5-yl)oxy)-N-ethyl-5-fluoro-N-isopropylbenzamide

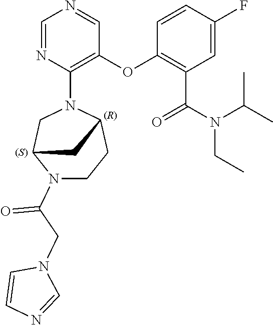

Off-white solid (34 mg, 33%).

¹H NMR (400 MHz, DMSO-d₆, 300 K) δ 8.40-8.30 (m, 1H), 7.95-7.80 (m, 1H), 7.55-7.45 (m, 1H), 7.35-7.20 (m, 2H), 7.08-6.93 (m, 2H), 6.85 (s, 1H), 5.15-5.00 (m, 2H), 4.95-4.80 (m, 1H), 4.72-4.60 (m, 1H), 4.50-4.08 (m, 1H), 3.83-3.76 (m, 3H), 3.76-3.65 (m, 1H), 3.25-3.10 (m, 2H), 2.95-2.80 (m, 1H), 2.00-1.90 (m, 2H), 1.85-1.69 (m, 2H), 1.25-1.00 (m, 9H). m/z: ES⁺ [M+H]⁺=522.40.

### Example 1.115: N-ethyl-5-fluoro-N-isopropyl-2-((4-((1S,5R)-2-(2-(4-methyl-1H-imidazol-1-yl)acetyl)-2,6-diazabicyclo[3.2.1]octan-6-yl)pyrimidin-5-yl)oxy)benzamide

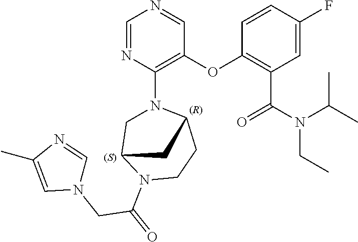

Colourless solid (20 mg, 16%).

¹H NMR (400 MHz, DMSO-d₆, 300 K) δ 8.44-8.26 (m, 1H), 7.95-7.71 (m, 1H), 7.69-7.448 (m, 1H), 7.41-7.38 (m, 2H), 7.14-6.92 (m, 1H), 6.96-6.81 (m, 1H), 5.12-4.94 (m, 2H), 4.93-4.76 (m, 1H), 4.69-4.32 (m, 1H), 4.19-3.67 (m, 3H), 3.37-2.68 (m, 4H), 2.21-2.08 (m, 3H), 1.99-1.78 (m, 2H), 1.71-7.56 (m, 2H), 1.23-0.99 (m, 9H). m/z: ES⁺ [M+H]⁺=535.62.

### Example 1.116: N-ethyl-5-fluoro-N-isopropyl-2-((4-((1S,5R)-2-(2-(4-methyl-1H-imidazol-1-yl)acetyl)-2,6-diazabicyclo[3.2.1]octan-6-yl)pyrimidin-5-yl)oxy)benzamide

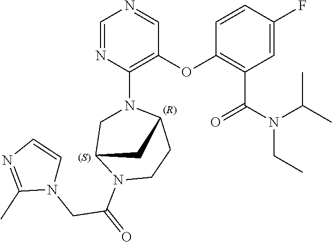

Colourless solid (20 mg, 16%).

¹H NMR (400 MHz, DMSO-d₆, 300 K) δ 8.45-8.23 (m, 1H), 7.96-7.72 (m, 1H), 7.41-7.21 (m, 2H), 7.16-6.85 (m, 2H), 6.74-6.58 (m, 1H), 5.12-4.91 (m, 2H), 4.91-4.60 (m, 2H), 4.45-3.57 (m, 4H), 3.30-2.64 (m, 2H), 2.23-2.09 (m, 3H), 1.99-1.81 (m, 2H), 1.75-1.59 (m, 2H), 1.30-0.90 (m, 9H). m/z: ES⁺ [M+H]⁺=535.62.

### Example 1.117: 2-((4-((1S,5R)-2-((R)-2-(1H-imidazol-1-yl)propanoyl)-2,6-diazabicyclo[3.2.1]octan-6-yl)pyrimidin-5-yl)oxy)-N-ethyl-5-fluoro-N-isopropylbenzamide

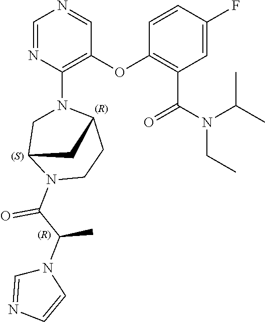

Colourless solid, TFA salt (92 mg, 58%₀).

¹H NMR (400 MHz, DMSO-d₆, 300 K) δ 9.30-9.20 (m, 1H), 8.54 (s, 1H), 8.20-7.80 (m, 1H), 7.78-7.60 (m, 1H), 7.60-7.40 (m, 2H), 7.40-7.00 (m, 1H), 5.95-5.83 (m, 1H), 5.20-5.09 (m, 1H), 5.00-4.80 (m, 2H), 4.40-4.20 (m, 1H), 4.00-3.80 (m, 3H), 3.60-3.40 (m, 1H), 3.30-3.20 (m, 1H), 2.90-2.70 (m, 1H), 2.02-1.80 (m, 3H), 1.87-1.70 (m, 4H), 1.30-0.80 m, 9H). m/z: ES⁺ [M+H]⁺=536.20.

### Example 1.118: 2-((4-((1S,5R)-2-((S)-2-(1H-imidazol-1-yl)propanoyl)-2,6-diazabicyclo[3.2.1]octan-6-yl)pyrimidin-5-yl)oxy)-N-ethyl-5-fluoro-N-isopropylbenzamide

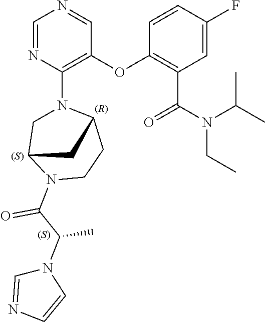

Colourless solid (45 mg, 45%).

¹H NMR (400 MHz, DMSO-d₆, 300 K) δ 8.38-8.26 (m, 1H), 7.91-7.65 (m, 2H), 7.33-7.10 (m, 3H), 7.09-6.81 (m, 3H), 5.71-5.45 (m, 1H), 5.31-4.05 (m, 3H), 3.91-3.49 (m, 3H), 3.43-3.3 (m, 3H), 2.91-2.73 (m, 1H), 2.65-1.75 (m, 2H), 1.73-1.33 (m, 4H), 1.31-0.92 (m, 9H). m/z: ES⁺ [M+H]⁺=536.30.

### Example 1.119: N-ethyl-5-fluoro-N-isopropyl-2-((4-((1S,5R)-2-(2-(5-methyl-1H-imidazol-1-yl)acetyl)-2,6-diazabicyclo[3.2.1]octan-6-yl)pyrimidin-5-yl)oxy)benzamide

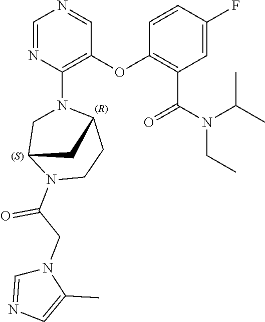

Colourless solid (11 mg, 12%).

¹H NMR (400 MHz, DMSO-d₆, 300 K) δ 8.46-8.28 (m, 1H), 7.98-7.76 (m, 2H), 7.45-7.31 (m, 2H), 7.20-6.98 (m, 1H), 6.96-6.81 (m, 1H), 5.12-4.94 (m, 2H), 4.93-4.76 (m, 1H), 4.69-4.32 (m, 1H), 4.19-3.67 (m, 3H), 3.37-2.68 (m, 4H), 2.21-2.08 (m, 3H), 1.99-1.78 (m, 2H), 1.71-7.56 (m, 2H), 1.23-0.99 (m, 9H). m/z: ES⁺ [M+H]⁺=535.62.

### Example 1.120: N-ethyl-5-fluoro-N-isopropyl-2-((4-((1S,5R)-2-(2-(1-methyl-1H-imidazol-4-yl)acetyl)-2,6-diazabicyclo[3.2.1]octan-6-yl)pyrimidin-5-yl)oxy)benzamide

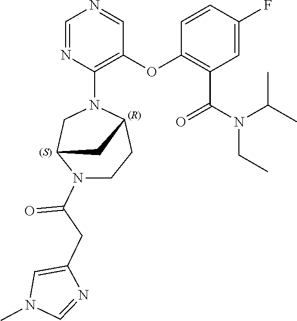

Off-white solid (16 mg, 30%).

¹H NMR (400 MHz, DMSO-d₆, 300 K) δ 8.35-8.25 (m, 1H), 7.83-7.67 (m, 1), 7.49 (s, 1H), 7.42-7.20 (m, 2H), 7.10-6.84 (m, 2H), 5.15-5.05 (m, 1H), 4.90-4.70 (m, 1H), 4.44-4.10 (m, 2H), 3.95-3.70 (m, 3H), 3.45-3.30 (m, 3H), 3.28-3.01 (m, 3H), 2.75-2.60 (m, 1H), 1.96-1.73 (m, 2H), 1.70-1.50 (m, 2H), 1.20-0.94 (m, 9H). m/z: ES⁺ [M+H]⁺=535.62.

### Example 1.121: N-ethyl-5-fluoro-N-isopropyl-2-((4-((1S,5R)-2-(2-(1-methyl-1H-imidazol-2-yl)acetyl)-2,6-diazabicyclo[3.2.1]octan-6-yl)pyrimidin-5-yl)oxy)benzamide

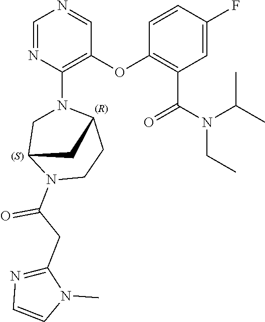

Off-white solid (22 mg, 25%).

¹H NMR (400 MHz, DMSO-d₆, 300 K) δ 8.70-8.60 (m, 1H), 8.05-7.95 (m, 1H), 7.25-7.15 (m, 2H), 7.05-7.00 (m, 2H), 7.00-6.92 (m, 1H), 5.22-5.12 (m, 2H), 4.80-4.20 (s, 1H), 4.19-3.82 (m, 5H), 3.75-3.60 (m, 3H), 3.33-3.20 (m, 1H), 3.18-3.10 (m, 1H), 2.05-1.90 (m, 2H), 1.21-1.12 (m, 2H), 1.10-0.96 (m, 9H). m/z: ES⁺ [M+H]⁺=536.62.

### Example 1.122: N-ethyl-5-fluoro-N-isopropyl-2-((4-((1S,5R)-2-(2-(1-methyl-1H-imidazol-5-yl)acetyl)-2,6-diazabicyclo[3.2.1]octan-6-yl)pyrimidin-5-yl)oxy)benzamide

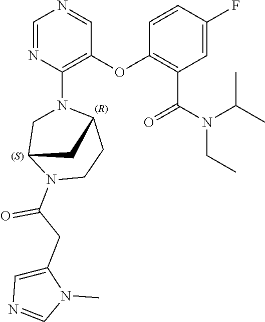

Colourless solid, TFA salt (41 mg, 63%).

¹H NMR (400 MHz, DMSO-d₆, 300 K) δ 9.03 (s, 1H), 8.55 (s, 1H), 8.18-7.70 (m, 1H), 7.60-7.42 (m, 1H), 7.41-7.06 (m, 2H), 5.28-4.92 (m, 1H), 4.88-4.78 (m, 1H), 4.08-4.06 (m, 1H), 4.04-4.02 (m, 1H), 3.88-3.86 (m, 1H), 3.83-3.81 (m, 2H), 3.70 (s, 3H), 3.41-3.32 (m, 1H), 3.29-3.09 (m, 2H), 3.08-2.74 (m, 1H), 2.10-1.91 (m, 2H), 1.88-1.52 (m, 2H), 1.25-0.98 (m, 9H). m/z: ES⁺ [M+H]⁺=536.30.

### Example 1.123: 2-((4-((1S,5R)-2-(2-(1H-1,2,4-triazol-1-yl)acetyl)-2,6-diazabicyclo[3.2.1]octan-6-yl)pyrimidin-5-yl)oxy)-N-ethyl-5-fluoro-N-isopropylbenzamide

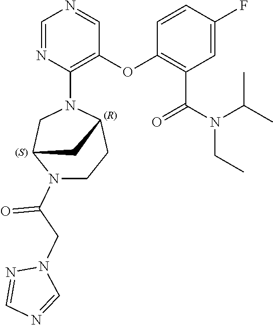

Colourless solid (23 mg, 65%).

m/z: ES⁺ [M+H]⁺=523.3.

### Example 1.124: N-ethyl-5-fluoro-N-isopropyl-2-((4-((1S,5R)-2-(1-methyl-1H-1,2,3-triazole-5-carbonyl)-2,6-diazabicyclo[3.2.1]octan-6-yl)pyrimidin-5-yl)oxy)benzamide

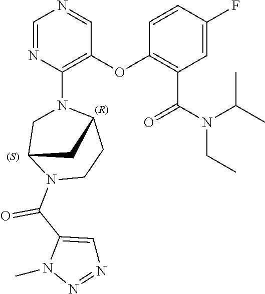

Colourless solid (21 mg, 59%).

m/z: ES⁺ [M+H]⁺=523.3.

### Example 1.125: N-ethyl-5-fluoro-N-isopropyl-2-((4-((1S,5R)-2-(1-methyl-1H-1,2,4-triazole-5-carbonyl)-2,6-diazabicyclo[3.2.1]octan-6-yl)pyrimidin-5-yl)oxy)benzamide

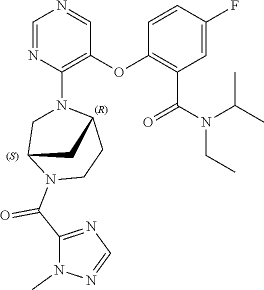

Colourless solid (17 mg, 50%).

m/z: ES⁺ [M+H]⁺=523.3.

### Example 1.126: N-ethyl-5-fluoro-N-isopropyl-2-((4-((1S,5R)-2-(pyrimidine-4-carbonyl)-2,6-diazabicyclo[3.2.1]octan-6-yl)pyrimidin-5-yl)oxy)benzamide

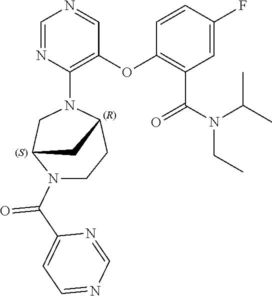

Colourless solid (18 mg, 51%).

m/z: ES⁺ [M+H]⁺=520.2.

### Example 1.127: N-ethyl-5-fluoro-2-((4-((1S,5R)-2-(5-fluoronicotinoyl)-2,6-diazabicyclo[3.2.1]octan-6-yl)pyrimidin-5-yl)oxy)-N-isopropylbenzamide

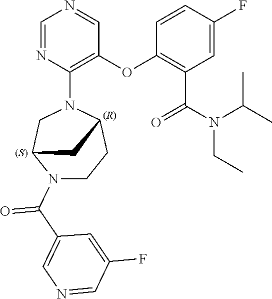

Colourless solid (20 mg, 56%).

m/z: ES⁺ [M+H]⁺=537.3.

### Example 1.128: N-ethyl-5-fluoro-N-isopropyl-2-((4-((1S,5R)-2-(pyridazine-4-carbonyl)-2,6-diazabicyclo[3.2.1]octan-6-yl)pyrimidin-5-yl)oxy)benzamide

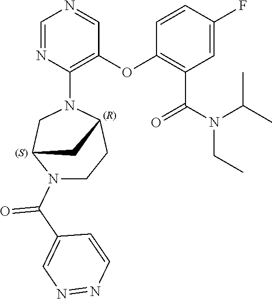

Colourless solid (17 mg, 48%).

m/z: ES⁺ [M+H]⁺=520.3.

### Example 1.129: N-ethyl-5-fluoro-N-isopropyl-2-((4-((1S,5R)-2-(pyrimidine-2-carbonyl)-2,6-diazabicyclo[3.2.1]octan-6-yl)pyrimidin-5-yl)oxy)benzamide

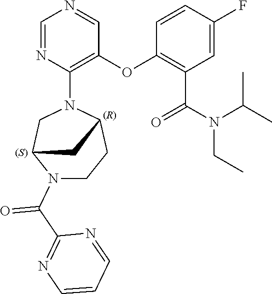

Colourless solid (14 mg, 41%).

m/z: ES⁺ [M+H]⁺=520.2.

### Example 1.130: N-ethyl-5-fluoro-N-isopropyl-2-((4-((1S,5R)-2-(pyrimidine-5-carbonyl)-2,6-diazabicyclo[3.2.1]octan-6-yl)pyrimidin-5-yl)oxy)benzamide

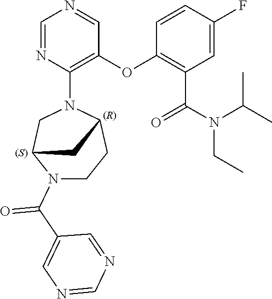

Colourless solid (17 mg, 48%).

m/z: ES⁺ [M+H]⁺=520.2.

### Example 1.131: N-ethyl-5-fluoro-N-isopropyl-2-((4-((1S,5R)-2-(pyridazine-3-carbonyl)-2,6-diazabicyclo[3.2.1]octan-6-yl)pyrimidin-5-yl)oxy)benzamide

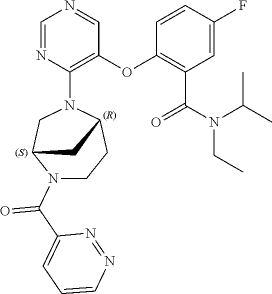

Colourless solid (22 mg, 62%).

m/z: ES⁺ [M+H]⁺=520.2.

### Example 1.132: N-ethyl-5-fluoro-N-isopropyl-2-((4-((1S,5R)-2-(pyrazine-2-carbonyl)-2,6-diazabicyclo[3.2.1]octan-6-yl)pyrimidin-5-yl)oxy)benzamide

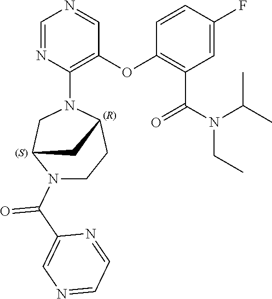

Colourless solid (14 mg, 40%).

m/z: ES⁺ [M+H]⁺=520.3.

### Example 1.133: 2-((4-((1S,5R)-2-(1H-1,2,4-triazole-5-carbonyl)-2,6-diazabicyclo[3.2.1]octan-6-yl)pyrimidin-5-yl)oxy)-N-ethyl-5-fluoro-N-isopropylbenzamide

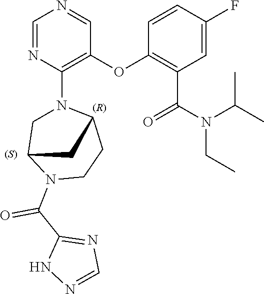

Colourless solid (34 mg, 99%).

m/z: ES⁺ [M+H]⁺=509.2.

### Example 1.134: N-ethyl-5-fluoro-N-isopropyl-2-((4-((1S,5R)-2-(1-methyl-1H-imidazole-2-carbonyl)-2,6-diazabicyclo[3.2.1]octan-6-yl)pyrimidin-5-yl)oxy)benzamide

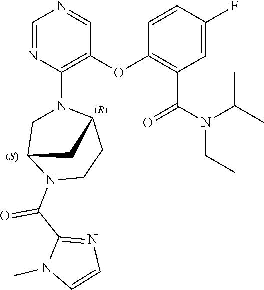

Colourless solid (13 mg, 38%).

m/z: ES⁺ [M+H]⁺=522.3.

### Example 1.135: 2-((4-((1S,5R)-2-(1H-imidazole-2-carbonyl)-2,6-diazabicyclo[3.2.1]octan-6-yl)pyrimidin-5-yl)oxy)-N-ethyl-5-fluoro-N-isopropylbenzamide

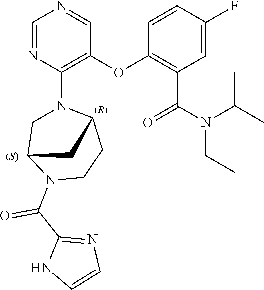

Colourless solid (13 mg, 39%).

m/z: ES⁺ [M+H]⁺=508.2.

### Example 1.136: N-ethyl-5-fluoro-N-isopropyl-2-((4-((1S,5R)-2-(oxazole-4-carbonyl)-2,6-diazabicyclo[3.2.1]octan-6-yl)pyrimidin-5-yl)oxy)benzamide

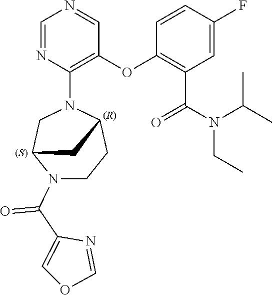

Colourless solid (16 mg, 47%).

m/z: ES⁺ [M+H]⁺=509.3.

### Example 1.137: 2-((4-((1S,5R)-2-(1H-pyrazole-5-carbonyl)-2,6-diazabicyclo[3.2.1]octan-6-yl)pyrimidin-5-yl)oxy)-N-ethyl-5-fluoro-N-isopropylbenzamide

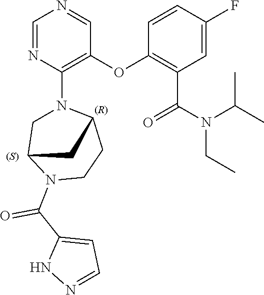

Colourless solid (13 mg, 40%).

m/z: ES⁺ [M+H]⁺=508.2.

### Example 1.138: 2-((4-((1S,5R)-2-(1H-imidazole-5-carbonyl)-2,6-diazabicyclo[3.2.1]octan-6-yl)pyrimidin-5-yl)oxy)-N-ethyl-5-fluoro-N-isopropylbenzamide

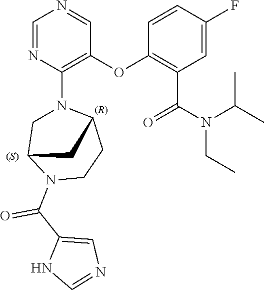

Colourless solid (12 mg, 36%).

m/z: ES⁺ [M+H]⁺=508.2.

### Example 1.139: N-ethyl-5-fluoro-N-isopropyl-2-((4-((1S,5R)-2-(1-methyl-1H-pyrazole-5-carbonyl)-2,6-diazabicyclo[3.2.1]octan-6-yl)pyrimidin-5-yl)oxy)benzamide

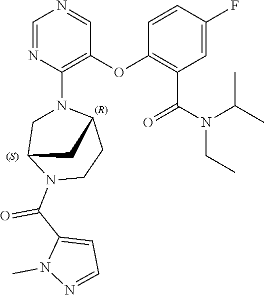

Colourless solid (19 mg, 56%).

m/z: ES⁺ [M+H]⁺=522.3.

### Example 1.140: N-ethyl-5-fluoro-2-((4-((1S,5R)-2-(2-hydroxy-2-methylpropanoyl)-2,6-diazabicyclo[3.2.1]octan-6-yl)pyrimidin-5-yl)oxy)-N-isopropylbenzamide

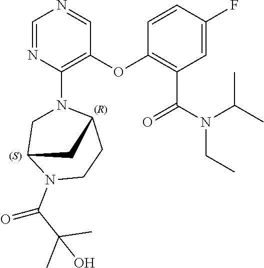

Colourless solid (62 mg, 51%).

¹H NMR (400 MHz, DMSO-d₆, 300 K) δ 8.40-8.25 (m, 1H), 7.90-7.71 (m, 1H), 7.35-7.18 (m, 2H), 7.15-6.87 (m, 1H), 5.79-4.98 (m, 2H), 4.90-4.05 (m, 2H), 3.95-3.50 (m, 3H), 3.35-3.06 (m, 2H), 2.67 (s, 1H), 1.92-1.47 (m, 4H), 1.37-1.22 (m, 6H), 1.20-0.96 (m, 9H). m/z: ES⁺ [M+H]⁺=500.25.

### Example 1.141: N-ethyl-5-fluoro-2-((4-((1S,5R)-2-((S)-2-hydroxypropanoyl)-2,6-diazabicyclo[3.2.1]octan-6-yl)pyrimidin-5-yl)oxy)-N-isopropylbenzamide

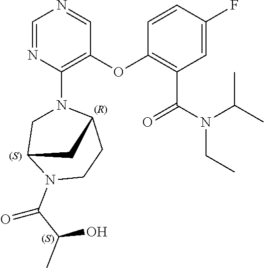

Colourless solid (29 mg, 25%).

¹H NMR (400 MHz, DMSO-d₆, 300 K) δ 8.37-8.30 (m, 1H), 7.90-7.75 (m, 1H), 7.35-7.20 (m, 2H), 7.15-6.97 (m, 1H), 5.10-4.95 (m, 1H), 4.90-4.75 (m, 2H), 4.64-4.19 (m, 2H), 4.00-3.78 (m, 3H), 3.65-3.50 (m, 1H), 3.25-3.05 (m, 2H), 2.00-1.18 (m, 4H), 1.20-0.98 (m, 12H). m/z: ES⁺ [M+H]⁺=486.56.

### Example 1.142: N-ethyl-5-fluoro-2-((4-((1S,5R)-2-((R)-2-hydroxypropanoyl)-2,6-diazabicyclo[3.2.1]octan-6-yl)pyrimidin-5-yl)oxy)-N-isopropylbenzamide

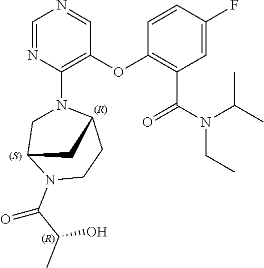

Colourless solid (21 mg, 17%).

¹H NMR (400 MHz, DMSO-d₆, 300 K) δ 8.40-8.20 (m, 1H), 7.96-7.74 (m, 1H), 7.52-7.16 (m, 2H), 7.16-6.72 (m, 1H), 5.22-4.98 (m, 1H), 4.88-4.78 (m, 1H), 4.68-4.60 (m, 2H), 4.00-3.58 (m, 4H), 3.48-3.42 (m, 1H), 3.32-2.98 (m, 2H), 2.08-1.32 (m, 4H), 1.20-1.12 (m, 4H), 1.12-1.08 (m, 4H), 1.08-0.90 (m, 4H). m/z: ES⁺ [M+H]⁺=486.56.

### Example 1.143: 2-((4-((1S,5R)-2-(2-(difluoromethoxy)acetyl)-2,6-diazabicyclo[3.2.1]octan-6-yl)pyrimidin-5-yl)oxy)-N-ethyl-5-fluoro-N-isopropylbenzamide

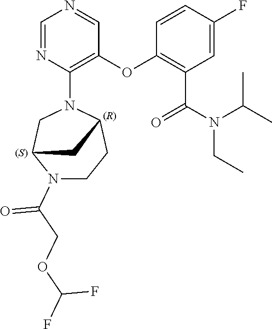

Colourless oil (4 mg, 11%).

m/z: ES⁺ [M+H]⁺=522.2.

### Example 1.144: N-Ethyl-5-fluoro-N-isopropyl-2-((4-((1S,5R)-2-(2-((R)-piperidin-3-yl)acetyl)-2,6-diazabicyclo[3.2.1]octan-6-yl)pyrimidin-5-yl)oxy)benzamide

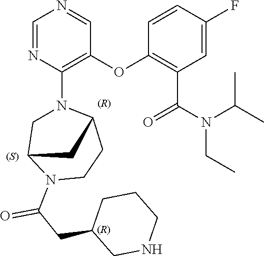

Colourless solid (43 mg, 35% over 2 steps).

¹H NMR (400 MHz, DMSO-d₆, 300 K) δ 8.36-8.28 (m, 1H), 7.88-7.73 (m, 1H), 7.34-7.18 (m, 2H), 6.90-6.82 (m, 1H), 5.56-4.20 (m, 3H), 3.95-3.67 (m, 3H), 3.66-3.46 (m, 1H), 3.43-3.37 (m, 1H), 3.22-2.98 (m, 2H), 2.93-2.59 (m, 2H), 2.47-2.34 (m, 1H), 2.31-2.00 (m, 3H), 1.98-1.64 (m, 5H), 1.64-1.44 (m, 2H), 1.41-1.24 (m, 1H), 1.56-0.80 (m, 10H). m/z: ES⁺ [M+H]⁺=539.50.

### Example 1.145: N-Ethyl-5-fluoro-N-isopropyl-2-((4-((1S,5R)-2-(2-((S)-piperidin-3-yl)acetyl)-2,6-diazabicyclo[3.2.1]octan-6-yl)pyrimidin-5-yl)oxy)benzamide

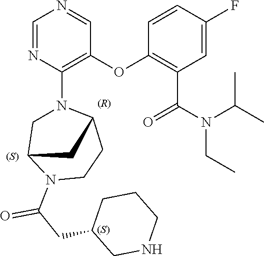

Colourless solid (32 mg, 30% over 2 steps).

¹H NMR (400 MHz, DMSO-d₆, 300 K) δ 8.40-8.24 (m, 1H), 7.85-7.74 (m, 1H), 7.33-7.18 (m, 2H), 7.09-6.90 (m, 1H), 5.19-4.77 (m, 1H), 4.73-4.10 (m, 2H), 3.93-3.66 (m, 3H), 3.65-3.46 (m, 2H), 3.28-3.05 (m, 2H), 2.92-2.58 (m, 2H), 2.45-2.31 (m, 1H), 2.28-2.04 (m, 3H), 1.97-1.45 (m, 7H), 1.38-1.24 (m, 1H), 1.23-0.96 (m, 10H). m/z: ES⁺ [M+H]⁺=539.15.

### Example 1.146 and 1.147: 2-((4-((1S,5R)-2-((S)-D-valyl)-2,6-diazabicyclo[3.2.1]octan-6-yl)pyrimidin-5-yl)oxy)-N-ethyl-5-fluoro-N-isopropylbenzamide and 2-((4-((1S,5R)-2-((R)-L-valyl)-2,6-diazabicyclo[3.2.1]octan-6-yl)pyrimidin-5-yl)oxy)-N-ethyl-5-fluoro-N-isopropylbenzamide

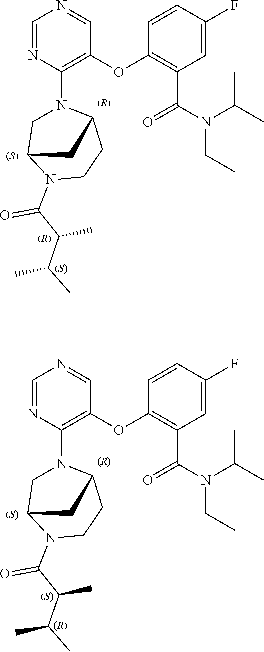

Separation by chiral HPLC gave two single stereoisomers:

Peak #1: Colourless solid (11 mg, 6% over 2 steps).

¹H NMR (400 MHz, DMSO-d₆, 300 K) 8.40-8.20 (m, 1H), 7.86-7.70 (m, 1H), 7.38-7.13 (m, 2H), 7.11-6.88 (m, 1H), 5.22-5.16 (m, 1H), 4.87-4.78 (m, 1H), 4.78-4.25 (m, 1H), 4.25-3.87 (m, 1H), 3.87-3.70 (m, 2H), 3.70-3.42 (m, 2H), 3.24-3.00 (m, 2H), 3.00-2.80 (m, 1H), 2.78-2.60 (m, 1H), 2.00-1.78 (m, 2H), 1.78-1.49 (m, 2H), 1.29-0.80 (m, 16H). m/z: ES⁺ [M+H]⁺=513.25.

Peak #2: Colourless solid (8 mg, 5% over 2 steps).

¹H NMR (400 MHz, DMSO-d₆, 300 K) δ 8.40-8.24 (m, 1H), 7.90-7.71 (m, 1H), 7.35-7.19 (m, 2H), 7.09-6.83 (m, 1H), 5.25-5.10 (m, 1H), 4.90-4.49 (m, 2H), 4.47-3.88 (m, 1H), 3.88-3.55 (m, 3H), 3.22-3.09 (m, 2H), 3.09-2.87 (m, 1H), 2.80-2.60 (m, 1H), 2.08-1.78 (m, 2H), 1.78-1.49 (m, 2H), 1.23-0.80 (m, 17H). m/z: ES⁺ [M+H]⁺=513.25.

### Example 1.148: 2-((4-((1S,5R)-2-((2R,5S)-2,5-dimethylpyrrolidine-2-carbonyl)-2,6-diazabicyclo[3.2.1]octan-6-yl)pyrimidin-5-yl)oxy)-N-ethyl-5-fluoro-N-isopropylbenzamide and 2-((4-((1S,5R)-2-((2S,5S)-2,5-dimethylpyrrolidine-2-carbonyl)-2,6-diazabicyclo[3.2.1]octan-6-yl)pyrimidin-5-yl)oxy)-N-ethyl-5-fluoro-N-isopropylbenzamide

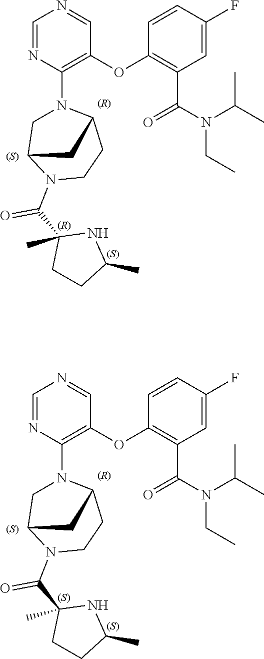

Colourless solid (16 mg, 28% over 2 steps).

¹H NMR (400 MHz, DMSO-d₆, 300 K) δ 8.36-8.30 (m, 1H), 7.90-7.73 (m, 1H), 7.33-7.16 (m, 2H), 7.08-6.92 (m, 1H), 5.28-5.06 (m, 1H), 4.87-4.20 (m, 2H), 3.87-3.52 (m, 2H), 3.27-3.06 (m, 3H), 2.43-2.26 (m, 1H), 1.94-1.78 (m, 2H), 1.73-1.37 (m, 4H), 3.09-2.66 (m, 4H), 1.29-1.22 (m, 3H), 1.20-1.14 (m, 2H), 1.13-1.08 (m, 4H), 1.08-0.94 (m, 6H). m/z: ES⁺ [M+H]⁺=539.15.

### Example 1.149: N-ethyl-5-fluoro-N-isopropyl-2-((4-((1S,5R)-2-((R)-3-methyl-2-azabicyclo[2.1.1]hexane-3-carbonyl)-2,6-diazabicyclo[3.2.1]octan-6-yl)pyrimidin-5-yl)oxy)benzamide and N-ethyl-5-fluoro-N-isopropyl-2-((4-((1S,5R)-2-((S)-3-methyl-2-azabicyclo[2.1.1]hexane-3-carbonyl)-2,6-diazabicyclo[3.2.1]octan-6-yl)pyrimidin-5-yl)oxy)benzamide

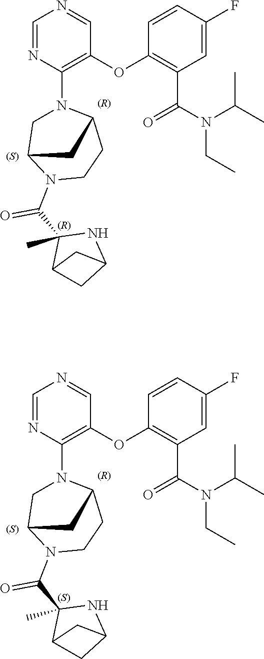

Colourless solid (9 mg, 10% over 2 steps).

¹H NMR (400 MHz, DMSO-d₆, 300 K) δ 8.40-8.25 (m, 1H), 7.93-7.67 (m, 1H), 7.37-7.16 (m, 2H), 7.14-6.88 (m, 1H), 5.48-5.03 (m, 1H), 4.94-4.59 (m, 1H), 4.52-4.10 (m, 1H), 4.04-3.60 (m, 3H), 3.0-3.54 (m, 4H), 3.55-3.68 (m, 3H), 2.75-2.61 (m, 1H), 2.04-1.78 (m, 2H), 1.74-1.48 (m, 4H), 1.45-1.17 (m, 5H), 1.15-0.84 (m, 9H). m/z: ES⁺ [M+H]⁺=537.35.

### Example 1.150: 2-((4-((1S,5R)-2-(1-azabicyclo[3.3.1]nonane-5-carbonyl)-2,6-diazabicyclo[3.2.1]octan-6-yl)pyrimidin-5-yl)oxy)-N-ethyl-5-fluoro-N-isopropylbenzamide

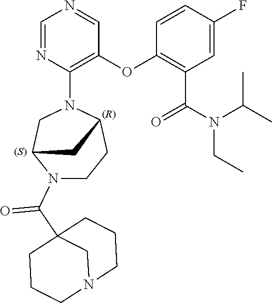

Colourless solid (25 mg, 13%).

¹H NMR (400 MHz, DMSO-d₆, 300K) δ 8.37-8.27 (m, 1H), 7.89-7.70 (m, 1H), 7.35-7.18 (m, 2H), 7.07-6.93 (m, 1H), 5.15-5.00 (m, 1H), 4.87-4.28 (m, 1H), 4.11-3.96 (m, 1H), 3.93-3.47 (m, 3H), 3.45-3.35 (m, 1H), 3.29-3.10 (m, 1H), 2.94-2.66 (m, 7H), 2.35-2.17 (m, 2H), 1.99-1.81 (m, 4H), 1.72-1.50 (m, 4H), 1.44-1.26 (m, 2H), 1.22-0.97 (m, 9H). m/z: ES⁺ [M+H]⁺=565.35.

### Example 1.151: N-ethyl-5-fluoro-N-isopropyl-2-((4-((1S,5R)-2-((3aR,6aR)-2-methyloctahydrocyclopenta[c]pyrrole-3a-carbonyl)-2,6-diazabicyclo[3.2.1]octan-6-yl)pyrimidin-5-yl)oxy)benzamide

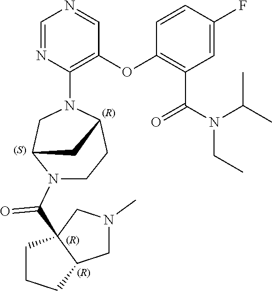

Light yellow solid, formic acid salt (30 mg, 15%)

¹H NMR (400 MHz, DMSO-d₆, 300 K) δ 8.32-8.22 (m, 2H), 7.83-7.77 (m, 1H), 7.28-7.22 (m, 2H), 7.10-6.89 (m, 1H), 5.10-4.80 (m, 1H), 4.30-4.18 (m, 1H), 3.79-3.43 (m, 5H), 3.38-2.66 (m, 5H), 2.40-2.31 (m, 4H), 2.05-1.86 (m, 4H), 1.68-1.62 (m, 5H), 1.56-1.41 (m, 2H), 1.15-1.06 (m, 9H). m/z: ES⁺ [M+H]⁺=565.36.

### Example 1.152: 2-((4-((1S,5R)-2-((3aR,6aR)-5-benzylhexahydro-1H-furo[3,4-c]pyrrole-3a-carbonyl)-2,6-diazabicyclo[3.2.1]octan-6-yl)pyrimidin-5-yl)oxy)-N-ethyl-5-fluoro-N-isopropylbenzamide and 2-((4-((1S,5R)-2-((3aS,6aS)-5-benzylhexahydro-1H-furo[3,4-c]pyrrole-3a-carbonyl)-2,6-diazabicyclo[3.2.1]octan-6-yl)pyrimidin-5-yl)oxy)-N-ethyl-5-fluoro-N-isopropylbenzamide

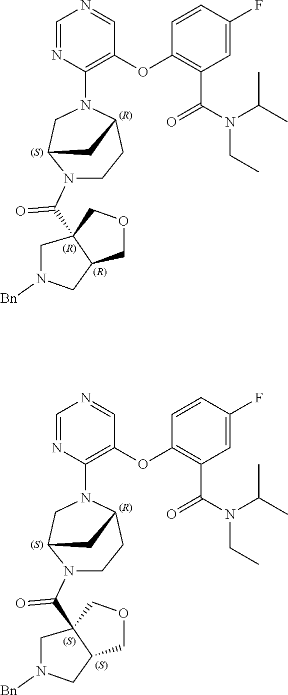

Colourless solid (55 mg, 21%).

¹H NMR (400 MHz, DMSO-d₆, 300 K) 8.37-8.28 (m, 1H), 7.87-7.81 (m, 1H), 7.35-7.19 (m, 7H), 7.10-6.99 (m, 1H), 5.11 (m, 1H), 4.82-4.17 (m, 2H), 4.02-3.69 (m, 5H), 3.65-3.52 (m, 4H), 3.51-3.42 (m, 2H), 3.39 (m, 1H), 3.24-3.13 (m, 1H), 2.86-2.75 (m, 2H), 2.32-2.10 (m, 2H), 2.05-1.85 (m, 2H), 1.61-1.32 (m, 2H), 1.19-0.95 (m, 9H). m/z: ES⁺ [M+H]⁺=643.35.

### Example 1.153: N-ethyl-5-fluoro-N-isopropyl-2-((4-((1S,5R)-2-((R)-2-(methoxymethyl)pyrrolidine-2-carbonyl)-2,6-diazabicyclo[3.2.1]octan-6-yl)pyrimidin-5-yl)oxy)benzamide and N-ethyl-5-fluoro-N-isopropyl-2-((4-((1S,5R)-2-((S)-2-(methoxymethyl)pyrrolidine-2-carbonyl)-2,6-diazabicyclo[3.2.1]octan-6-yl)pyrimidin-5-yl)oxy)benzamide

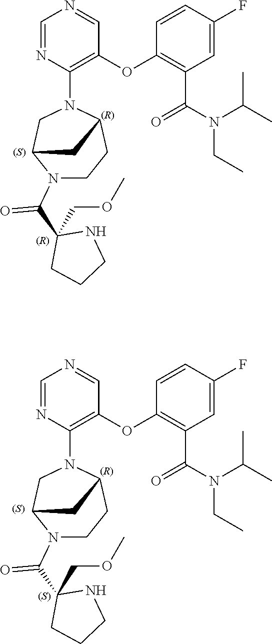

Colourless solid (36 mg, 17% over 2 steps). ¹H NMR (400 MHz, CDCl₃, 300 K) δ 8.49-8.35 (m, 1H), 7.93-7.79 (m, 1H), 7.07-6.94 (m, 2H), 6.84-6.64 (m, 1H), 5.50-2.66 (m, 16H), 2.20-1.53 (m, 8H), 1.36-1.00 (m, 9H). m/z: ES⁺ [M+H]⁺=555.4.

### Examples 1.154 and 1.155: 2-((4-((1S,5R)-2-((1R,5R)-3-azabicyclo[3.2.0]heptane-1-carbonyl)-2,6-diazabicyclo[3.2.1]octan-6-yl)pyrimidin-5-yl)oxy)-N-ethyl-5-fluoro-N-isopropylbenzamide and 2-((4-((1S,5R)-2-((1S,5S)-3-azabicyclo[3.2.0]heptane-1-carbonyl)-2,6-diazabicyclo[3.2.1]octan-6-yl)pyrimidin-5-yl)oxy)-N-ethyl-5-fluoro-N-isopropylbenzamide

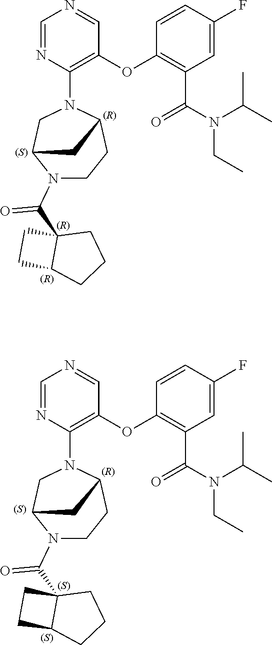

Colourless solid, HCl salt (140 mg, 60% over 2 steps). ¹H NMR (400 MHz, DMSO-d₆, 296.3 K) δ 10.41-9.89 (m, 2H), 8.66 (s, 1H), 8.15-7.85 (m, 1H), 7.54-7.12 (m, 3H), 5.43-4.65 (m, 2H), 4.53-3.52 (m, 6H), 3.52-2.77 (m, 5H), 2.45-2.14 (m, 3H), 2.12-1.67 (m, 6H), 1.35-0.65 (m, 9H). m/z: ES⁺ [M+H]⁺=537.30.

Separation by chiral HPLC gave two stereoisomers:

Peak #1: Rt=9.55 min, colourless solid (28 mg, 25%).

¹H NMR (400 MHz, DMSO-d₆, 296 K) δ 8.45-8.36 (m, 1H), 7.94-7.76 (m, 1H), 7.36-7.15 (m, 2H), 7.15-6.87 (m, 1H), 5.21-4.82 (m, 1H), 4.78-4.02 (m, 2H), 3.95-3.54 (m, 3H), 3.21-2.94 (m, 4H), 2.74-2.63 (m, 3H), 2.24-1.78 (m, 6H), 1.74-1.32 (m, 4H), 1.31-0.97 (m, 9H). m/z: ES⁺ [M+H]⁺=537.30.

Peak #2: Rt=12.93 min, colourless solid (40 mg, 36%).

¹H NMR (400 MHz, DMSO-d₆, 296 K) δ 8.45-8.36 (m, 1H), 7.94-7.76 (m, 1H), 7.36-7.15 (m, 2H), 7.15-6.87 (m, 1H), 6.51-4.78 (m, 2H), 4.76-4.02 (m, 2H), 3.95-3.54 (m, 3H), 3.21-2.94 (m, 4H), 2.74-2.63 (m, 3H), 2.24-1.78 (m, 6H), 1.74-1.32 (m, 4H), 1.31-0.67 (m, 9H). m/z: ES⁺ [M+H]⁺=537.25.

### Examples 2.01 to 2.33

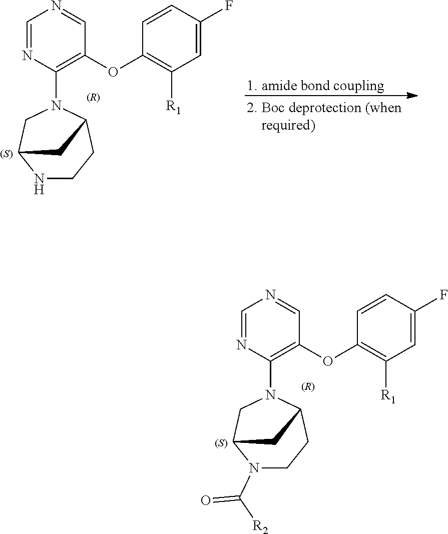

wherein R1 can be as defined anywhere herein

| | | Amide bond | | Boc | |
|---|---|---|---|---|---|
| | Carboxylic acid | coupling | Step#1 | deprotection | Step#2 |
| Ex. | reagent | method | purification | procedure | purification |
| 2.01 | (S)-1-(tert- | A1 | RPFC1, 10% to 100% | B2 | Prep-HPLC1; |
| | butoxycarbonyl)-2- | | MeCN gradient in 20 | | Gradient: 18% B to |
| | methylpyrrolidine- | | min | | 43% B in 7 min; |
| | 2-carboxylic acid | | | | Rt = 6.33 min |
| 2.02 | (S)-1-(tert- | A1 | RPFC1, 10% to 100% | B2 | Prep-HPLC1; |
| | butoxycarbonyl)-2- | | MeCN gradient in 20 | | Gradient: 20% B to |
| | ethylpyrrolidine- | | min | | 45% B in 7 min; |
| | 2-carboxylic acid | | | | Rt = 6.33 min |
| 2.03 | N-(tert- | A1 | Prep-TLC | B2 | Prep-HPLC1; |
| | butoxycarbonyl)- | | (DCM/MeOH 30:1) | | Gradient: 24% B to |
| | N-methylglycine | | | | 41% B in 8 min; |
| | | | | | Rt = 6.45 min |
| 2.04 | N-(tert- | A1 | RPFC1, 0% to 100% | B2 | RPFC2, 0% to |
| | butoxycarbonyl)- | | MeCN gradient in 15 | | 100% MeCN |
| | N-methylglycine | | min | | gradient in 20 min |
| 2.05 | (tert- | A1 | Prep-TLC | B2 | Prep-HPLC1; |
| | butoxycarbonyl)- | | (DCM/MeOH 30:1) | | Gradient: 20% B to |
| | L-proline | | | | 41% B in 7 min: |
| | | | | | Rt = 6.45 min |
| 2.06 | (tert- | A1 | RPFC1, 0% to 100% | B2 | RPFC2, 0% to |
| | butoxycarbonyl)- | | MeCN gradient in 15 | | 100% MeCN |
| | L-proline | | min | | gradient in 20 min |
| 2.07 | (S)-1-(tert- | A1 | Silica gel FC, eluting | B2 | RPFC1, 50% to |
| | butoxycarbonyl)- | | with PE/EtOAc (1:1) | | 100% MeCN |
| | 2-methylpyrrolidine- | | | | gradient in 30 min |
| | 2-carboxylic acid | | | | |
| 2.08 | dimethylglycine | A1 | Prep-HPLC14; | — | — |
| | | | Gradient: 13% B to | | |
| | | | 38% B in 8 min; | | |
| | | | Rt = 8.02 min | | |
| 2.09 | (S)-1-(tert- | A1 | RPFC1, 10% to 50% | B2 | Prep-HPLC1; |
| | butoxycarbonyl)- | | MeCN gradient in 10 | | Gradient: 29% B to |
| | 2-methylpyrrolidine- | | min | | 54% B in 7 min; |
| | 2-carboxylic acid | | | | Rt = 6.07 min |
| 2.10 | (S)-1-(tert- | A1 | Silica gel, | B1 | RPFC1, 10% to |
| | butoxycarbonyl)-2- | | DCM/MeOH (10:1) | | 50% MeCN |
| | methylpyrrolidine- | | | | gradient in 10 min |
| | 2-carboxylic acid | | | | |
| 2.11 | (S)-1-(tert- | A1 | Silica gel, | B1 | Prep-HPLC1; |
| | butoxycarbonyl)- | | DCM/MeOH (12:1) | | Gradient: 24% B to |
| | 2-ethylpyrrolidine- | | | | 38% B in 7 min; |
| | 2-carboxylic acid | | | | Rt = 6.43 min |
| 2.12 | 7-(tert- | A1 | RPFC1, 60% to 70% | B1 | Prep-HPLC10; |
| | butoxycarbonyl)-7- | | MeCN gradient in 10 | | Gradient: 5% B to |
| | azabicyclo[2.2.1] | | min | | 18% B in 8 min; |
| | heptane-1- | | | | Rt = 7.4 min |
| | carboxylic acid | | | | |
| 2.13 | (R)-3-((tert- | A1 | RPFC1, 0% to 100% | B1 | Prep-HPLC1; |
| | butoxycarbonyl)am- | | MeCN gradient in 30 | | Gradient: 17% B to |
| | ino)butanoic | | min | | 31% B in 7 min; |
| | acid | | | | Rt = 7.0 min |
| 2.14 | (R)-3-((tert-butoxy- | A1 | Prep-TLC | B2 | RPFC1, 0% to |
| | carbonyl)(methyl)am- | | (DCM/MeOH, 20:1) | | 100% MeCN |
| | ino)butanoic acid | | | | gradient in 10 min |
| 2.15 | tetrahydro-1H- | A1 | RPFC1, 10% to 50% | — | — |
| | pyrrolizine-7a(5H)- | | MeCN gradient in 10 | | |
| | carboxylic acid | | min | | |
| 2.16 | (S)-1-(tert- | A1 | RPFC1, 10% to 90% | B2 | Prep-HPLC1; |
| | butoxycarbonyl)-2- | | MeCN gradient in 10 | | Gradient: 22% B to |
| | methylpyrrolidine- | | min | | 48% B in 7 min; |
| | 2-carboxylic acid | | | | Rt = 6.22 min |
| 2.17 | (1S,3S,5S)-2-(tert- | A1 | RPFC1, 10% to 100% | B1 | Prep-HPLC1; |
| | butoxycarbonyl)-2- | | MeCN gradient in 20 | | Gradient: 22% B to |
| | azabicyclo[3.1.0] | | min | | 44% B in 7 min; |
| | hexane-3- | | | | Rt = 6.8 min |
| | carboxylic acid | | | | |
| 2.18 | (S)-1-(tert- | A1 | RPFC1, 40% to 90% | B1 | Prep-HPLC1; |
| | butoxycarbonyl)-2- | | MeCN gradient in 15 | | Gradient: 21% B to |
| | methylpyrrolidine- | | min | | 36% B in 7 min; |
| | 2-carboxylic acid | | | | Rt = 6.88 min |
| 2.19 | (S)-1-(tert- | A1 | — | B1 | Prep-HPLC6; |
| | butoxycarbonyl)-2- | | | | Gradient: 3% B to |
| | ethylpyrrolidine- | | | | 20% B in 7 min; |
| | 2-carboxylic acid | | | | Rt = 6.5 min |
| 2.20 | tetrahydro-1H- | A1 | Prep-HPLC17; | — | — |
| | pyrrolizine-7a(5H)- | | Gradient: 13% B to | | |
| | carboxylic acid | | 43% B in 10 min; | | |
| | | | Rt = 12.18 min | | |
| 2.21 | (3aRS,6aRS)-2- | A1 | RPFC1, 10% to 100% | B2 | Prep-HPLC1; |
| | (tert- | | MeCN gradient in 20 | | Gradient: 22% B to |
| | butoxycarbonyl)- | | min | | 48% B in 7 min; |
| | hexahydro- | | | | Rt = 6.22 min; |
| | cyclopenta[c]pyrrole- | | | | CHIRALPAK IC; |
| | 3a-carboxylic acid | | | | A: Hex(0.2% DEA), |
| | | | | | B: IPA:DCM = 1:1; |
| | | | | | gradient: isocratic; |
| | | | | | Rt = 9.97 min |
| 2.22 | (3aRS,6aRS)-2- | A1 | RPFC1, 10% to 100% | B2 | Prep-HPLC1; |
| | (tert- | | MeCN gradient in 20 | | Gradient: 22% B to |
| | butoxycarbonyl)- | | min | | 48% B in 7 min; |
| | hexahydro- | | | | Rt = 6.22 min; |
| | cyclopenta[c]pyrrole- | | | | CHIRALPAK IC; |
| | 3a-carboxylic acid | | | | A: Hex(0.2% DEA), |
| | | | | | B: IPA:DCM = 1:1; |
| | | | | | gradient: isocratic; |
| | | | | | Rt = 15.87 min |
| 2.23 | (1S,2S,5R)-3- | A1 | RPFC1, 50% to 70% | B1 | Prep-HPLC19; |
| | (tert- | | MeCN gradient in 10 | | Gradient: 2% B to |
| | butoxycarbonyl)-3- | | min; | | 22% B in 78 min; |
| | azabicyclo[3.1.0] | | | | Rt = 8.27 min |
| | hexane-2- | | | | |
| | carboxylic acid | | | | |
| 2.24 | imidazoleacetic | A1 | Prep-HPLC18; | — | — |
| | acid | | Gradient: 18% B to | | |
| | | | 48% B in 10 min; | | |
| | | | Rt = 10.38 min | | |
| 2.25 | dimethylglycine | A1 | Prep-HPLC1; | — | — |
| | | | Gradient: 20% B to | | |
| | | | 43% B in 7 min; | | |
| | | | Rt = 6.33 min | | |
| 2.26 | (S)-1-(tert- | A1 | RPFC5, 10% to 50% | B1 | Prep-HPLC1; |
| | butoxycarbonyl)-2- | | MeCN gradient in 10 | | Gradient: 17% B to |
| | methylpyrrolidine- | | min; | | 42% B in 8 min; |
| | 2-carboxylic acid | | | | Rt = 6.95 min |
| 2.27 | (S)-1-(tert- | A1 | RPFC1, 10% to 100% | B2 | Prep-HPLC1; |
| | butoxycarbonyl)-2- | | MeCN gradient in 20 | | Gradient: 12% B to |
| | methylpyrrolidine- | | min | | 42% B in 8 min; |
| | 2-carboxylic acid | | | | Rt = 7.4 min |
| 2.28 | (3aR,6aR)-2-(tert- | A1 | RPFC1, 10% to 100% | B2 | Trituration with |
| | butoxycarbonyl)hexa- | | MeCN gradient in 20 | | EtOAc |
| | hydrocyclopenta[c]pyr- | | min | | |
| | role-3a(1H)- | | | | |
| | carboxylic acid | | | | |
| 2.29 | (3aR,6aR)-2-(tert- | A1 | RPFC1, 10% to 100% | B2 | Trituration with |
| | butoxycarbonyl)hexa- | | MeCN gradient in 10 | | EtOAc |
| | hydrocyclopenta[c]pyr- | | min | | |
| | role-3a(1H)- | | | | |
| | carboxylic acid | | | | |
| 2.30 | (3aR,6aR)-2-(tert- | A1 | RPFC1, 10% to 100% | B2 | Precipitated from |
| | butoxycarbonyl)hexa- | | MeCN gradient in 10 | | dioxane solution, |
| | hydrocyclopenta[c]pyr- | | min | | then washed with |
| | role-3a(1H)- | | | | EtOAc |
| | carboxylic acid | | | | |
| 2.31 | (S)-1-(tert- | A1 | RPFC1, 10% to 100% | B2 | Triturated with |
| | butoxycarbonyl)-2- | | MeCN gradient in 20 | | EtOAc |
| | methylpyrrolidine- | | min | | |
| | 2-carboxylic acid | | | | |
| 2.32 | (S)-1-(tert- | A1 | RPFC1, 30% to 70% | B2 | 1. Precipitation |
| | butoxycarbonyl)-2- | | MeCN gradient in 25 | | from DCM/Dioxane. |
| | methylpyrrolidine- | | min | | 2. CHIRALPAK |
| | 2-carboxylic acid | | | | IH, isocratic |
| | | | | | Hex(0.2% IPAmine): |
| | | | | | (EtOH:DCM = |
| | | | | | 1:1) = 30:70; |
| | | | | | Rt = 4.32 min |
| 2.33 | (S)-1-(tert- | A1 | RPFC1, 30% to 70% | B2 | 1. Precipitation |
| | butoxycarbonyl)-2- | | MeCN gradient in 25 | | from DCM/Dioxane. |
| | methylpyrrolidine- | | min | | 2. CHIRALPAK |
| | 2-carboxylic acid | | | | IH, isocratic |
| | | | | | Hex(0.2% IPAmine): |
| | | | | | (EtOH:DCM = |
| | | | | | 1:1) = 30:70; |
| | | | | | Rt = 7.02 min |
| 2.34 | (S)-1-(tert- | A1 | Silica gel | B2 | Precipitation from |
| | butoxycarbonyl)-2- | | chromatography, | | EtOAc/MeOH |
| | methylpyrrolidine- | | PE/EtOAc (1:1) | | (10:1) |
| | 2-carboxylic acid | | | | |
| 2.35 | (S)-1-(tert- | A1 | RPFC1, 50% to 70% | B2 | 1. Prep-HPLC2, |
| | butoxycarbonyl)-2- | | MeCN gradient in 10 | | Gradient 47% B to |
| | methylpyrrolidine- | | min | | 72% B in 12 min; |
| | 2-carboxylic acid | | | | Rt = 10.8 min. |
| | | | | | 2. CHIRALPAKIK3 |
| | | | | | isocratic A: |
| | | | | | Hex(0.2% IPAmine), |
| | | | | | B: EtOH:DCM = 1:1; |
| | | | | | 25% B; Rt = 18.33 |
| 2.36 | (S)-1-(tert- | A1 | RPFC1, 50% to 70% | B2 | 1. Prep-HPLC2, |
| | butoxycarbonyl)-2- | | MeCN gradient in 10 | | Gradient 47% B to |
| | methylpyrrolidine- | | min | | 72% B in 12 min; |
| | 2-carboxylic acid | | | | Rt = 10.8 min. |
| | | | | | 2. CHIRALPAKIK3 |
| | | | | | isocratic A: |
| | | | | | Hex(0.2% IPAmine), |
| | | | | | B: EtOH:DCM = 1:1; |
| | | | | | 25% B; Rt = 22.35 |

### Example 2.01: N-ethyl-N-(5-fluoro-2-((4-((1S,5R)-2-((S)-2-methylpyrrolidine-2-carbonyl)-2,6-diazabicyclo[3.2.1]octan-6-yl)pyrimidin-5-yl)oxy)phenyl)isobutyramide

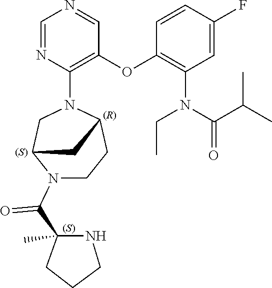

Colourless solid (54 mg, 34% over 2 steps).

¹H NMR (400 MHz, DMSO-d₆, 300 K) δ 8.55-8.20 (m, 1H), 7.98-7.65 (m, 1H), 7.52-7.35 (m, 1H), 7.30-7.09 (m, 1H), 7.05-6.82 (m, 1H), 4.95-5.85 (m, 1H), 4.95-4.65 (m, 1H), 4.60-4.15 (m, 1H), 4.05-3.90 (m, 1H), 3.85-3.73 (m, 1H), 3.70-3.50 (m, 1H), 3.50-3.35 (m, 1H), 3.30-2.95 (m, 2H), 2.85-2.75 (m, 1H), 2.65-2.55 (m, 1H), 2.45-2.30 (m, 1H), 2.20-2.05 (m, 1H), 2.05-1.75 (m, 2H), 1.72-1.52 (m, 5H), 1.32-1.15 (m, 3H), 1.15-1.00 (m, 4H), 1.00-0.92 (m, 3H), 0.95-0.85 (m, 3H). m/z: ES⁺ [M+H]⁺=525.30.

### Example 2.02: N-ethyl-N-(5-fluoro-2-((4-((1S,5R)-2-((S)-2-ethylpyrrolidine-2-carbonyl)-2,6-diazabicyclo[3.2.1]octan-6-yl)pyrimidin-5-yl)oxy)phenyl)isobutyramide

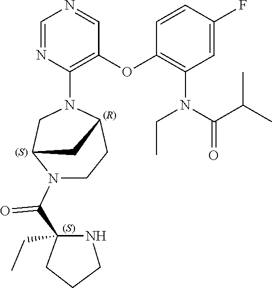

Off-white solid (33 mg, 42% over 2 steps).

¹H NMR (400 MHz, DMSO-d₆, 300 K) δ 8.50-8.22 (m, 1H), 7.95-7.65 (m, 1H), 7.60-7.35 (m, 1H), 7.35-7.10 (m, 1H), 7.10-6.82 (m, 1H), 5.35-5.05 (m, 1H), 4.95-4.60 (m, 1H), 4.52-4.15 (m, 1H), 4.10-3.90 (m, 1H), 3.87-3.65 (m, 1H), 3.65-3.40 (m, 1H), 3.30-3.15 (m, 1H), 3.15-2.95 (m, 1H), 2.95-2.72 (m, 2H), 2.60-2.50 (m, 1H), 2.40-2.35 (m, 1H), 2.20-2.05 (m, 1H), 1.98-1.72 (m, 2H), 1.70-1.52 (m, 7H), 1.15-1.05 (m, 3H), 1.02-0.95 (m, 3H), 0.95-0.82 (m, 3H), 0.80-0.55 (m, 3H). m/z: ES⁺ [M+H]⁺=539.35.

### Example 2.03: 1-((1S,5R)-6-(5-(2-(2-Cyclopropyl-5-methyl-1H-imidazol-1-yl)-4-fluorophenoxy)pyrimidin-4-yl)-2,6-diazabicyclo[3.2.1]octan-2-yl)-2-(methylamino)ethan-1-one

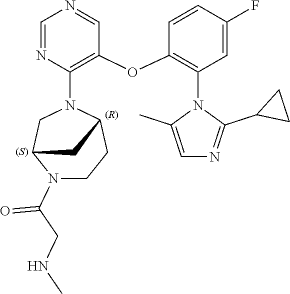

Colourless solid (40 mg, 43% over 2 steps).

¹H NMR (400 MHz, DMSO-d₆, 300 K) δ 8.34 (s, 1H), 7.93-7.88 (m, 1H), 7.60-7.56 (m, 1H), 7.43-7.39 (m, 1H), 7.31-7.07 (m, 1H), 6.57-6.55 (m, 1H), 5.28-3.78 (m, 3H), 3.77-3.40 (m, 3H), 3.28-3.10 (m, 1H), 3.07-2.88 (m, 1H), 2.79-2.60 (m, 1H), 2.24 (d, J=4.0 Hz, 3H), 2.08-1.92 (m, 3H), 1.89-1.65 (m, 2H), 1.61-1.42 (m, 2H), 0.88-0.57 (m, 4H). m/z: ES⁺ [M+H]⁺=492.25.

### Example 2.04: 1-((1S,5R)-6-(5-(2-(2-cyclopropyl-5-methyl-1H-imidazol-1-yl)-3,4-difluorophenoxy)pyrimidin-4-yl)-2,6-diazabicyclo[3.2.1]octan-2-yl)-2-(methylamino)ethan-1-one

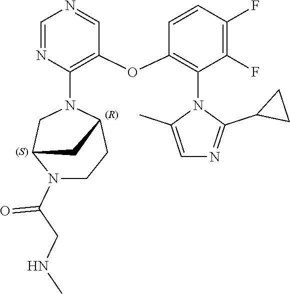

Colourless solid, formic acid salt (30 mg, 8% over 2 steps).

¹H NMR (400 MHz, DMSO-d₆, 300 K) δ 8.53-8.38 (m, 1H), 8.26-8.07 (m, 1H), 8.02-7.86 (m, 1H), 7.79-7.51 (m, 1H), 7.15-6.80 (m, 1H), 6.72-6.51 (m, 1H), 5.25-4.96 (m, 1H), 4.65-4.47 (m, 1H), 4.17-4.05 (m, 1H), 3.70-3.64 (m, 2H), 3.55-3.50 (m, 2H), 3.40-3.37 (m, 1H), 3.06-2.93 (m, 1H), 2.72-2.62 (m, 1H), 2.35-2.27 (m, 3H), 2.02-1.93 (m, 3H), 1.93-1.76 (m, 1H), 1.73-1.46 (m, 3H), 0.86-0.62 (m, 4H). m/z: ES⁺ [M+H]⁺=510.20.

### Example 2.05: (1S,5R)-2-(L-Prolyl)-6-(5-(2-(2-cyclopropyl-5-methyl-1H-imidazol-1-yl)-4-fluorophenoxy)pyrimidin-4-yl)-2,6-diazabicyclo[3.2.1]octane

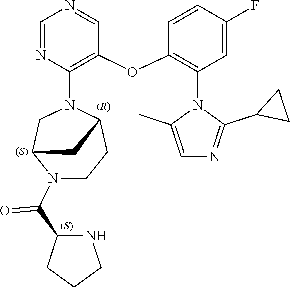

Colourless solid (39 mg, 39% over 2 steps).

¹H NMR (400 MHz, DMSO-d₆, 300 K) δ 8.33-8.32 (m, 1H), 7.91-7.89 (m, 1H), 7.59-7.56 (m, 1H), 7.44-7.39 (m, 1H), 7.21-7.16 (m, 1H), 6.57-6.56 (m, 1H), 5.11-3.88 (m, 4H), 3.79-3.43 (m, 3H), 3.29-3.12 (m, 1H), 3.03-2.89 (m, 1H), 2.79-2.58 (m, 3H), 2.18-1.94 (m, 4H), 1.92-1.68 (m, 4H), 1.65-1.30 (m, 5H), 0.99-0.71 (m, 4H). m/z: ES⁺ [M+H]⁺=518.25.

### Example 2.06: (1S,5R)-2-(L-prolyl)-6-(5-(2-(2-cyclopropyl-5-methyl-1H-imidazol-1-yl)-3,4-difluorophenoxy)pyrimidin-4-yl)-2,6-diazabicyclo[3.2.1]octane

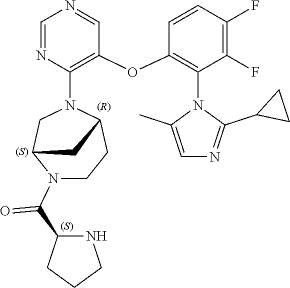

Yellow solid, formic acid salt (16 mg, 18% over 2 steps).

1H NMR (400 MHz, DMSO-d₆, 300 K) δ 8.50-8.32 (m, 1H), 8.32-8.12 (m, 1H), 8.09-7.88 (m, 1H), 7.75-7.59 (m, 1H), 7.12-6.91 (m, 1H), 6.72-6.51 (m, 1H), 5.22-5.00 (m, 1H), 4.77-4.47 (m, 2H), 4.20-3.99 (m, 2H), 3.76-3.67 (m, 2H), 3.08-2.98 (m, 2H), 2.87-2.69 (m, 2H), 2.18-1.93 (m, 4H), 1.93-1.80 (m, 1H), 1.78-1.67 (m, 2H), 1.67-1.42 (m, 4H), 0.89-0.63 (m, 4H). m/z: ES⁺ [M+H]⁺=536.15.

### Example 2.07: 5-fluoro-N-isopropyl-N-methyl-2-((4-((1S,5R)-2-((S)-2-methylpyrrolidine-2-carbonyl)-2,6-diazabicyclo[3.2.1]octan-6-yl)pyrimidin-5-yl)oxy)benzamide

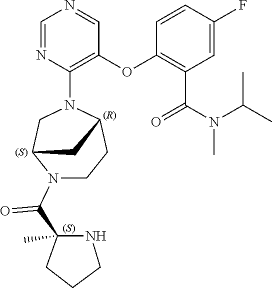

Yellow solid (35 mg, 16% over 2 steps).

1H NMR (400 MHz, DMSO-d₆, 300 K) δ 8.48-8.19 (m, 1H), 7.89-7.65 (m, 1H), 7.51-7.15 (m, 2H), 7.13-6.70 (m, 1H), 5.59-4.93 (m, 1H), 4.83-4.62 (m, 1H), 4.51-4.00 (m, 1H), 3.99-3.45 (m, 3H), 3.20-2.90 (m, 1H) 2.89-2.74 (m, 3H), 2.72-2.57 (m, 3H), 2.30-2.10 (m, 1H), 1.98-1.75 (m, 2H), 1.70-1.50 (m, 5H), 1.40-1.20 (m, 3H), 1.16-0.89 (m, 6H). m/z: ES⁺ [M+H]⁺=511.35.

### Example 2.08: 1-((1S,5R)-6-(5-(2-(2-cyclopropyl-1H-imidazol-1-yl)-4-fluorophenoxy)pyrimidin-4-yl)-2,6-diazabicyclo[3.2.1]octan-2-yl)-2-(dimethylamino)ethan-1-one

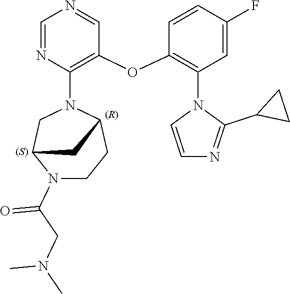

Yellow solid (30 mg, 19%).

¹H NMR (400 MHz, DMSO-d₆, 300 K) δ 8.35 (s, 1H), 7.93 (d, J=3.2 Hz, 1H), 7.65-7.55 (m, 1H), 7.44-7.33 (m, 1H), 7.25-7.20 (m, 1H), 7.20-7.10 (m, 1H), 6.83 (s, 1H), 5.10-4.80 (m, 1H), 4.65-4.50 (m, 1H), 4.58-3.85 (m, 1H), 3.75-3.63 (m, 2H), 3.03-2.93 (m, 3H), 2.13 (s, 6H), 1.95-1.80 (m, 1H), 1.75-1.60 (m, 2H), 1.60-1.50 (m, 2H), 0.90-0.75 (m, 4H). m/z: ES⁺ [M+H]⁺=492.20.

### Example 2.09: ((1S,5R)-6-(5-(4-fluoro-2-(2-isopropoxypyridin-3-yl)phenoxy)pyrimidin-4-yl)-2,6-diazabicyclo[3.2.1]octan-2-yl)((S)-2-methylpyrrolidin-2-yl)methanone

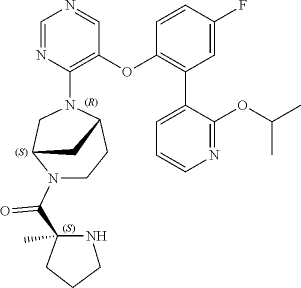

Colourless solid (26 mg, 45% over 2 steps).

¹H NMR (400 MHz, DMSO-d₆, 300 K) δ 8.33-8.22 (m, 1H), 8.20-8.12 (m, 1H), 7.83-7.68 (m, 1H), 7.70-7.63 (m, 1H), 7.31-7.22 (m, 2H), 7.12-7.06 (m, 1H), 7.02-6.94 (m, 1H), 5.36-5.01 (m, 2H), 4.64-4.45 (m, 1H), 4.39-4.03 (m, 1H), 3.78-3.62 (m, 1H), 3.01-2.77 (m, 2H), 2.70-2.53 (m, 1H), 2.34-2.07 (m, 2H), 1.85-1.73 (m, 1H), 1.73-1.47 (m, 5H), 1.44-1.28 (m, 1H), 1.27-1.15 (m, 3H), 1.14-1.02 (m, 6H). m/z: ES⁺ [M+H]⁺=546.65.

### Example 2.10: ((1S,5R)-6-(5-(2-(2-cyclopropyl-5-methyl-1H-imidazol-1-yl)-4-fluorophenoxy)pyrimidin-4-yl)-2,6-diazabicy clo[3.2.1]octan-2-yl)((S)-2-methylpyrrolidin-2-yl)methanone

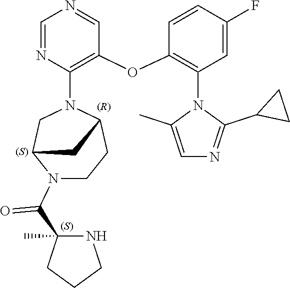

Colourless solid (29 mg, 38% over 2 steps).

¹H NMR (400 MHz, DMSO-d₆, 300 K) δ 8.34 (s, 1H), 7.99-7.83 (m, 1H), 7.68-7.51 (m, 1H), 7.51-7.32 (m, 1H), 7.41-7.01 (m, 1H), 6.69-6.48 (m, 1H), 5.44-4.98 (m, 1H), 4.73-4.47 (m, 1H), 4.43-4.01 (m, 1H), 3.89-3.57 (m, 1H), 3.58-3.41 (m, 1H), 3.14-2.90 (s, 1H), 2.89-2.75 (m, 1H), 2.72-2.56 (m, 1H), 2.30-2.10 (m, 1H), 2.00-1.90 (m, 3H), 1.89-1.71 (m, 2H), 1.70-1.31 (m, 7H), 1.21 (s, 3H), 0.83-0.58 (m, 4H). m/z: ES⁺ [M+H]⁺=532.20.

### Example 2.11: ((1S,5R)-6-(5-(2-(2-cyclopropyl-5-methyl-1H-imidazol-1-yl)-4-fluorophenoxy)pyrimidin-4-yl)-2,6-diazabicy clo[3.2.1]octan-2-yl)((S)-2-ethylpyrrolidin-2-yl)methanone

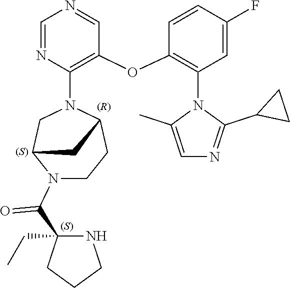

Off-white solid (33 mg, 42% over 2 steps).

¹H NMR (400 MHz, DMSO-d₆, 300 K) δ 8.35 (s, 1H), 7.91 (d, J=6.8 Hz, 1H), 7.68-7.52 (m, 1H), 7.50-7.31 (m, 1H), 7.29-6.88 (m, 1H), 6.63-6.40 (m, 1H), 5.35-5.01 (m, 1H), 4.95-4.40 (m, 1H), 4.39-4.13 (m, 1H), 3.90-3.60 (m, 1H), 3.59-3.36 (m, 2H), 3.12-2.82 (m, 1H), 2.80-2.72 (m, 1H), 2.65-2.52 (m, 1H), 2.20-2.02 (m, 1H), 2.01-1.92 (m, 3H), 1.91-1.75 (m, 1H), 1.75-1.32 (m, 9H), 0.85-0.60 (m, 7H). m/z: ES⁺ [M+H]⁺=546.35.

### Example 2.12: (7-azabicyclo[2.2.1]heptan-1-yl)((1S,5R)-6-(5-(2-(2-cyclopropyl-5-methyl-1H-imidazol-1-yl)-4-fluorophenoxy)pyrimidin-4-yl)-2,6-diazabicyclo[3.2.1]octan-2-yl)methanone

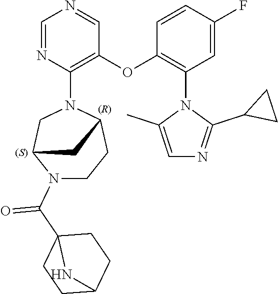

Off-white solid (22 mg, 26% over 2 steps).

¹H NMR (400 MHz, DMSO-d₆, 300 K) δ 8.34 (s, 1H), 7.91 (d, J=4.8 Hz, 1H), 7.67-7.49 (m, 1H), 7.48-7.31 (m, 1H), 7.30-6.91 (m, 1H), 6.56 (s, 1H), 6.00-4.95 (m, 1H), 4.90-4.03 (m, 2H), 3.81-3.64 (m, 1H), 3.61-3.40 (m, 2H), 3.10-2.89 (m, 1H), 2.66-2.54 (m, 1H), 2.01-1.90 (m, 3H), 1.88-1.63 (m, 6H), 1.62-1.45 (m, 5H), 1.43-1.28 (m, 2H), 0.84-0.58 (m, 4H). m/z: ES⁺ [M+H]⁺=544.30.

### Example 2.13: (3R)-3-amino-1-((1S,5R)-6-(5-(2-(2-cyclopropyl-5-methyl-1H-imidazol-1-yl)-4-fluorophenoxy)pyrimidin-4-yl)-2,6-diazabicyclo[3.2.1]octan-2-yl)butan-1-one

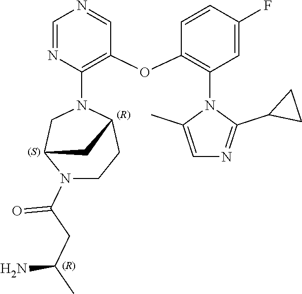

Off-white solid (46 mg, 38% over 2 steps).

¹H NMR (400 MHz, DMSO-d₆, 300 K) δ 8.34 (s, 1H), 8.02-7.80 (m, 1H), 7.71-7.50 (m, 1H), 7.49-7.30 (m, 1H), 7.29-7.02 (m, 1H), 6.69-6.45 (m, 1H), 5.21-4.96 (m 1H), 4.80-4.05 (m, 2H), 3.88-3.60 (m, 2H), 3.13-2.90 (m, 3H), 2.61-2.54 (m 1H), 2.36-2.30 (m 1H), 2.28-2.10 (m, 1H), 2.06-1.90 (m, 3H), 1.88-1.76 (m, 1H), 1.75-1.63 (m, 1H), 1.61-1.32 (m, 3H), 1.16-0.90 (m, 3H), 0.88-0.58 (m, 4H). m/z: ES⁺ [M+H]⁺=506.25.

### Example 2.14: (3R)-3-amino-1-((1S,5R)-(5-(2-(2-cyclopropyl-5-methyl-1H-imidazol-1-yl)-4-fluorophenoxy)pyrimidin-4-yl)-2,6-diazabicyclo[3.2.1]octan-2-yl)-3-(methylamino)butan-1-one

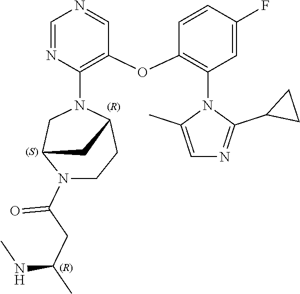

Off-white solid (29 mg, 36% over 2 steps).

¹H NMR (400 MHz, DMSO-d₆, 300 K) δ 8.39-8.21 (m, 1H), 7.98-7.81 (m, 1H), 7.66-7.49 (m, 1H), 7.47-7.30 (m, 1H), 7.29-7.00 (m, 1H), 6.66-6.45 (m, 1H), 5.19-4.99 (m, 1H), 4.79-4.02 (m, 2H), 3.75-3.62 (m, 1H), 3.55-3.41 (m, 1H), 3.12-2.96 (m, 1H), 2.89-2.72 (m, 1H), 2.71-2.56 (m, 1H), 2.47-2.34 (m, 1H), 2.29-2.18 (m, 3H), 2.18-2.04 (m, 1H), 2.03-1.92 (m, 3H), 1.89-1.65 (m, 2H), 1.64-1.32 (m, 3H), 1.13-0.84 (m, 3H), 0.83-0.62 (m, 4H). m/z: ES⁺ [M+H]⁺=520.25.

### Example 2.15: ((1S,5R)-6-(5-(2-(2-cyclopropyl-5-methyl-1H-imidazol-1-yl)-4-fluorophenoxy)pyrimidin-4-yl)-2,6-diazabicyclo[3.2.1]octan-2-yl)(tetrahydro-1H-pyrrolizin-7a(5H)-yl)methanone

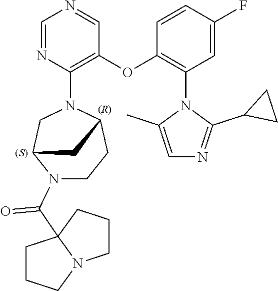

Off-white solid (22 mg, 35%).

¹H NMR (400 MHz, DMSO-d₆, 300 K) δ 8.34 (s, 1H), 7.91 (d, J=7.6 Hz 1H), 7.70-7.50 (m, 1H), 7.50-7.25 (m, 1H), 7.21-7.07 (m, 1H), 6.63-6.50 (m, 1H), 5.70-4.90 (m, 1H), 4.88-4.40 (m, 1H), 4.39-4.00 (m, 1H), 3.80-3.60 (m, 1H), 3.54-3.39 (m, 1H), 2.97-2.80 (m, 2H), 2.69-2.58 (m, 2H), 2.28-2.02 (m, 2H), 2.00-1.89 (m, 3H), 1.90-1.68 (m, 6H), 1.67-1.40 (m, 6H), 0.99-0.50 (m, 4H). m/z: ES⁺ [M+H]⁺=558.30.

### Example 2.16: ((1S,5S)-6-(5-(2-(4-cyclopropylpyridin-3-yl)-4-fluorophenoxy)pyrimidin-4-yl)-2,6-diazabicyclo[3.2.1]octan-2-yl)((S)-2-methylpyrrolidin-2-yl)methanone

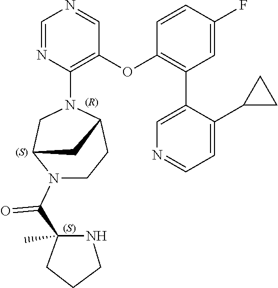

Off-white solid (9 mg, 19% over 2 steps).

1H NMR (400 MHz, DMSO-d₆, 300 K) δ 8.42-8.22 (m, 3H), 7.86 (s, 1H), 7.36-7.23 (m, 2H), 7.12-6.99 (m, 1H), 6.91-6.76 (m, 1H), 5.43-5.05 (m, 2H), 4.66-4.08 (m, 2H), 3.75-3.42 (m, 2H), 2.99-2.75 (m, 2H), 2.68-2.54 (m, 2H), 2.34-2.06 (m, 1H), 1.86-1.33 (m, 7H), 1.30-1.16 (m, 3H), 1.01-0.87 (m, 2H), 0.82-0.67 (m, 2H). m/z: ES⁺ [M+H]⁺=529.30.

### Example 2.17: ((1S,3S,5S)-2-azabicyclo[3.1.0]hexan-3-yl)((1S,5R)-6-(5-(2-(4-cyclopropylpyridin-3-yl)-4-fluorophenoxy)pyrimidin-4-yl)-2,6-diazabicyclo[3.2.1]octan-2-yl)methanone

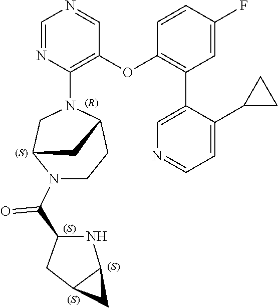

Off-white solid (22 mg, 9% over 2 steps).

¹H NMR (400 MHz, DMSO-d₆, 300 K) δ 8.41-8.36 (m, 1H), 8.36-8.32 (m, 1H), 8.30-8.26 (m, 1H), 7.93-7.72 (m, 1H), 7.39-7.22 (m, 2H), 7.16-6.94 (m, 1H), 6.91-6.78 (m, 1H), 5.15-4.98 (m, 1H), 4.61-4.46 (m, 1H), 4.30-.13 (m, 1H), 3.77-.51 (m, 2H), 2.99-2.77 (m, 1H), 2.71-2.59 (m, 1H), 2.34-2.32 (m, 1H), 1.88-1.74 (m, 2H), 1.72-1.62 (m, 2H), 1.59-1.50 (m, 1H), 1.46-1.29 (m, 3H), 1.03-0.90 (m, 2H), 0.83-0.71 (m, 2H), 0.58-0.39 (m, 1H), 0.26-0.11 (m, 1H). m/z: ES⁺ [M+H]⁺=527.20.

### Example 2.18: ((1S,5R)-6-(5-(2-(4-cyclopropylpyrimidin-5-yl)-4-fluorophenoxy)pyrimidin-4-yl)-2,6-diazabicyclo[3.2.1]octan-2-yl)((S)-2-methylpyrrolidin-2-yl)methanone

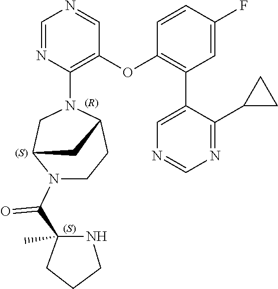

Off-white solid (9 mg, 11% over 2 steps).

¹H NMR (400 MHz, DMSO-d₆, 300 K) δ 9.05-8.86 (m, 1H), 8.62-8.54 (m, 1H), 8.31-8.22 (m, 1H), 8.01-7.82 (m, 1H), 7.55-7.42 (m, 1H), 7.41-7.25 (m, 1H), 7.13-6.85 (m, 1H), 5.62-4.79 (m, 1H), 4.68-3.89 (m, 2H), 3.86-3.43 (m, 2H), 3.05-2.86 (m, 2H), 2.67-2.58 (m, 1H), 2.26-2.10 (m, 1H), 1.98-1.70 (m, 2H), 1.74-1.35 (m, 6H), 1.35-1.15 (m, 3H), 1.14-0.87 (m, 4H). m/z: ES⁺ [M+H]⁺=530.30.

### Example 2.19: ((1S,5R)-6-(5-(2-(4-cyclopropylpyrimidin-5-yl)-4-fluorophenoxy)pyrimidin-4-yl)-2,6-diazabicyclo[3.2.1]octan-2-yl)((S)-2-ethylpyrrolidin-2-yl)methanone

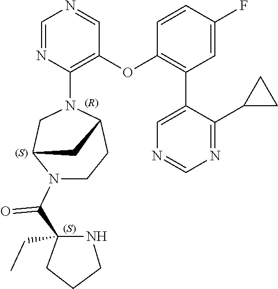

Off-white solid, trifluoroacetic acid salt (31 mg, 24% over 2 steps). 1H NMR (400 MHz, DMSO-d₆, 300 K) δ 9.10-8.90 (m, 1H), 8.65-8.54 (m, 1H), 8.51-8.30 (m, 1H), 8.1-7.88 (m, 1H), 7.52-7.25 (m, 2H), 7.21-6.91 (m, 1H), 5.28-5.01 (m, 1H), 4.78-4.48 (m, 1H), 4.25-4.13 (m, 1H), 3.98-3.63 (m, 2H), 3.26-3.17 (m, 2H), 3.15-2.72 (m, 2H), 2.45-2.16 (m, 2H), 2.14-2.01 (m, 1H), 2.00-1.72 (m, 6H), 1.71-1.41 (m, 2H), 1.15-0.95 (m, 4H), 0.94-0.54 (m, 3H). m/z: ES⁺ [M+H]⁺=544.15.

### Example 2.20: (1S,5R)-6-(5-(4-fluoro-2-(2-isopropyl-1H-imidazol-1-yl)phenoxy)pyrimidin-4-yl)-2,6-diazabicyclo[3.2.1]octan-2-yl)(tetrahydro-1H-pyrrolizin-7a(5H)-yl)methanone

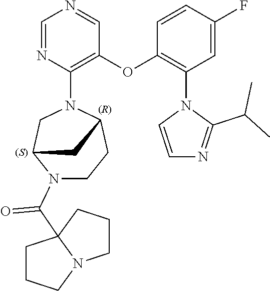

Colourless solid (22 mg, 25%).

¹H NMR (400 MHz, DMSO-d₆, 300 K) δ 8.32-8.30 (m, 1H), 8.00-7.90 (m, 1H), 7.80-7.60 (m, 1H), 7.50-7.39 (m, 1H), 7.25-7.21 (m, 1H), 7.18-7.14 (m, 1H), 7.01-6.90 (m, 1H), 5.50-4.92 (m, 1H), 4.82-4.32 (m, 1H), 4.25-4.14 (m, 1H), 3.82-3.69 (m, 1H), 3.05-2.96 (m, 2H), 2.90-2.83 (m, 1H), 2.75-2.63 (m, 2H), 2.60-2.50 (m, 2H), 2.30-2.12 (m, 2H), 1.92-1.73 (m, 5H), 1.70-1.47 (m, 5H), 1.32-1.12 (m, 6H). m/z: ES⁺ [M+H]⁺=546.30.

### Example 2.21 and 2.22: (1S,5R)-6-(5-(2-(2-cyclopropyl-5-methyl-1H-imidazol-1-yl)-4-fluorophenoxy)pyrimidin-4-yl)-2,6-diazabicyclo[3.2.1]octan-2-yl)((3aR,6aR)-hexahydrocyclopenta[c]pyrrol-3a(1H)-yl)methanone and (1S,5R)-6-(5-(2-(2-cyclopropyl-5-methyl-1H-imidazol-1-yl)-4-fluorophenoxy)pyrimidin-4-yl)-2,6-diazabicyclo[3.2.1]octan-2-yl)((3aS,6aS)-hexahydrocyclopenta[c]pyrrol-3a(1H)-yl)methanone

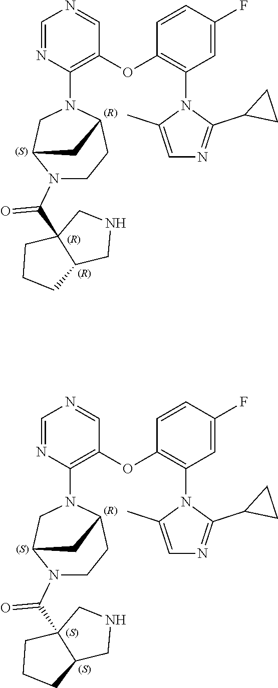

Separation by chiral HPLC gave two single stereoisomers:

Peak #1: colourless solid (17 mg, 20% over 2 steps).

1H NMR (400 MHz, DMSO-d₆, 300 K) δ 8.46-8.34 (m, 1H), 7.99-7.81 (m, 1H), 7.63-7.49 (m, 1H), 7.51-7.36 (m, 1H), 7.28-7.06 (m, 1H), 6.74-6.59 (m, 1H), 5.14-5.03 (m, 1H), 4.66-4.48 (m, 1H), 4.23-3.89 (m, 1H), 3.78-3.61 (m, 2H), 3.33-3.18 (m, 2H), 3.09-2.66 (m, 4H), 2.48-2.59 (m, 1H), 2.02-1.92 (m, 3H), 1.90-1.72 (m, 3H), 1.68-1.57 (m, 3H), 1.56-1.47 (m, 2H), 1.46-1.35 (m, 1H), 1.34-1.20 (m, 1H), 0.87-0.63 (m, 4H).

m/z: ES⁺ [M+H]⁺=558.15.

Peak #2: colourless solid (5 mg, 6% over 2 steps). 1H NMR (400 MHz, DMSO-d₆, 300 K) δ 8.46-8.34 (m, 1H), 7.99-7.81 (m, 1H), 7.63-7.49 (m, 1H), 7.51-7.36 (m, 1H), 7.28-7.06 (m, 1H), 6.74-6.59 (m, 1H), 5.14-5.03 (m, 1H), 4.66-4.48 (m, 1H), 4.23-3.89 (m, 1H), 3.78-3.61 (m, 2H), 3.33-3.18 (m, 2H), 3.09-2.66 (m, 4H), 2.48-2.59 (m, 1H), 2.02-1.92 (m, 3H), 1.90-1.72 (m, 3H), 1.68-1.57 (m, 3H), 1.56-1.47 (m, 2H), 1.46-1.35 (m, 1H), 1.34-1.20 (m, 1H), 0.87-0.63 (m, 4H). m/z: ES⁺ [M+H]⁺=558.10.

### Example 2.23: ((1S,2S,5R)-3-azabicyclo[3.1.0]hexan-2-yl)((1S,5R)-6-(5-(2-(2-cyclopropyl-5-methyl-1H-imidazol-1-yl)-4-fluorophenoxy)pyrimidin-4-yl)-2,6-diazabicyclo[3.2.1]octan-2-yl)methanone

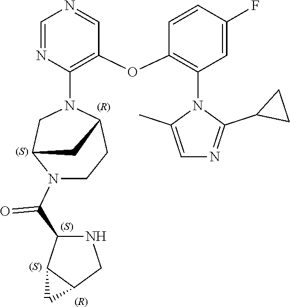

Light yellow oil, bis-formic acid salt (22 mg, 15% over 2 steps).

¹H NMR (400 MHz, DMSO-d₆, 300 K) δ 8.55-8.21 (m, 1H), 8.20-7.99 (m, 2H), 7.98-7.75 (m, 1H), 7.67-7.35 (m, 2H), 7.34-7.01 (m, 1H), 6.73-6.55 (m, 1H), 5.30-4.89 (m, 1H), 4.81-4.43 (m, 3H), 3.86-3.57 (m, 3H), 3.46-3.26 (m, 3H), 2.85-2.74 (m, 1H), 2.26-1.95 (m, 3H), 1.93-1.75 (m, 3H), 1.74-1.49 (m, 3H), 0.95-0.70 (m, 5H), 0.69-0.50 (m, 1H). m/z: ES⁺ [M+H]⁺=530.15.

### Example 2.24: 1-((1S,5R)-6-(5-(2-(2,5-dicyclopropyl-1H-imidazol-1-yl)-4-fluorophenoxy)pyrimidin-4-yl)-2,6-diazabicyclo[3.2.1]octan-2-yl)-2-(1H-imidazol-1-yl)ethan-1-one

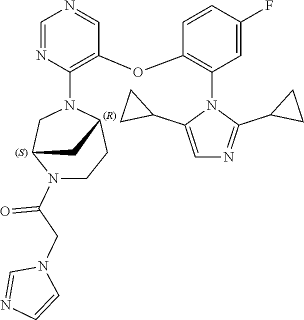

Colourless solid (51 mg, 24%).

¹H NMR (400 MHz, DMSO-d₆, 300 K) δ 8.36-8.35 (m, 1H), 8.00-7.90 (m, 1H), 7.64-7.62 (m, 1H), 7.51-7.50 (m, 1H), 7.43-7.41 (m, 1H), 7.35-7.16 (m, 1H), 7.02-7.00 (m, 1H), 6.85 (s, 1H), 6.49-6.47 (m, 1H), 5.14-4.95 (m, 2H), 4.84-4.83 (m, 1H), 4.67-4.65 (s, 1H), 4.20-3.44 (m, 3H), 3.19-2.58 (m, 1H), 1.89-1.80 (m, 1H), 1.73-1.65 (m, 2H), 1.58-1.30 (m, 3H), 0.89-0.62 (m, 6H), 0.57-0.41 (m, 2H). m/z: ES⁺ [M+H]⁺=555.25.

### Example 2.25: 1-((1S,5R)-6-(5-(2-(4-cyclopropylpyrimidin-5-yl)-4-fluorophenoxy)pyrimidin-4-yl)-2,6-diazabicyclo[3.2.1]octan-2-yl)-2-(dimethylamino)ethan-1-one

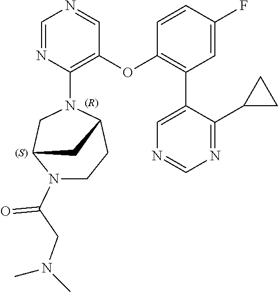

Off-white solid (17 mg, 18%).

1H NMR (400 MHz, DMSO-d₆, 300 K) δ 8.97 (s, 1H), 8.65-8.55 (m, 1H), 8.35-8.31 (m, 1H), 8.02-7.95 (m, 1H), 7.51-7.43 (m, 1H), 7.42-7.32 (m, 1H), 7.11-7.02 (m, 1H), 5.04-4.85 (m, 1H), 4.81-4.43 (m, 1H), 4.15-3.75 (m, 1H), 3.75-3.62 (m, 1H), 3.40-3.35 (m, 1H), 3.15-2.85 (m, 3H), 2.15-1.95 (m, 6H), 1.95-1.75 (m, 2H), 1.72-1.20 (m, 3H), 1.12-0.85 (m, 4H). m/z: ES⁺ [M+H]⁺=504.15.

### Example 2.26: ((1S,5R)-6-(5-(2-(2-cyclopropylpyridin-3-yl)-4-fluorophenoxy)pyrimidin-4-yl)-2,6-diazabicy clo[3.2.1]octan-2-yl)((S)-2-methylpyrrolidin-2-yl)methanone

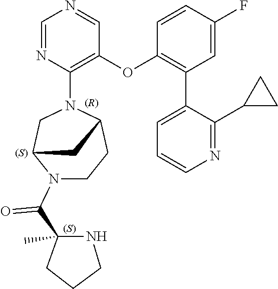

Colourless solid 11 mg, 11% over 2 steps).

¹H NMR (400 MHz, DMSO-d₆, 300 K) δ 8.40 (d, J=3.2 Hz, 1H), 8.27 (s, 1H), 7.95-7.73 (m, 1H), 7.65-7.51 (m, 1H), 7.39-7.24 (m, 2H), 7.23-7.11 (m, 1H), 7.10-6.90 (m, 1H), 5.49-5.00 (m, 1H), 4.70-4.48 (m, 1H), 4.47-4.00 (m, 1H), 3.80-3.60 (m, 1H), 3.60-3.40 (m, 1H), 3.30-3.25 (m, 1H), 3.10-2.73 (m, 2H), 2.63-2.54 (m, 1H), 2.30-2.10 (m, 1H), 1.88-1.70 (m, 2H), 1.69-1.32 (m, 6H), 1.24 (s, 3H), 0.92 (s, 2H), 0.89-0.70 (m 2H). m/z: ES⁺ [M+H]⁺=529.20.

### Example 2.27: ((1S,5R)-6-(5-(2-(3,5-dicyclopropyl-4H-1,2,4-triazol-4-yl)-4-fluorophenoxy)pyrimidin-4-yl)-2,6-diazabicyclo[3.2.1]octan-2-yl)((S)-2-methylpyrrolidin-2-yl)methanone

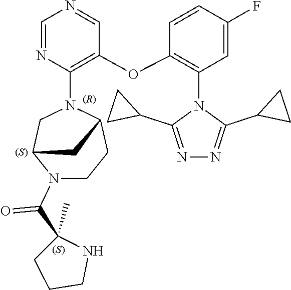

Yellow solid (15 mg, 12% over 2 steps).

¹H NMR (400 MHz, DMSO-d₆, 300K) δ 8.38 (s, 1H), 7.99 (s, 1H), 7.85-7.69 (m, 1H), 7.60-7.40 (m, 1H), 7.30-6.91 (m, 1H), 5.65-4.98 (m, 1H), 4.97-4.07 (m, 2H), 3.85-3.40 (m, 2H), 3.17-2.71 (m, 2H), 2.30-2.00 (m, 2H), 2.00-1.75 (m, 2H), 1.74-1.32 (m, 8H), 1.32-1.10 (m, 3H), 1.10-0.54 (m, 8H). m/z: ES⁺ [M+H]⁺=559.30.

### Example 2.28: ((1S,5R)-6-(5-(2-(3,5-dicyclopropyl-4H-1,2,4-triazol-4-yl)-4-fluorophenoxy)pyrimidin-4-yl)-2,6-diazabicyclo[3.2.1]octan-2-yl)((3aR,6aR)-hexahydrocyclopenta[c]pyrrol-3a(1H)-yl)methanone

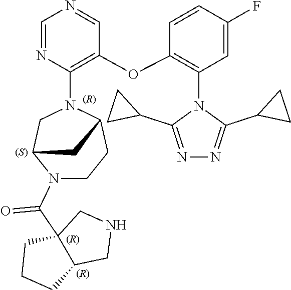

Yellow solid, HCl salt (39 mg, 32% over 2 steps).

¹H NMR (400 MHz, DMSO-d₆, 300K) δ 9.99-9.66 (m, 1H), 9.55-9.10 (m, 1H), 8.83-8.65 (m, 1H), 8.46-8.29 (m, 1H), 8.06-7.89 (m, 1H), 7.88-7.39 (m, 2H), 5.20-5.08 (m, 1H), 5.00-4.90 (m, 1H), 4.70-4.60 (m, 1H), 3.95-3.82 (m, 2H), 3.76-3.65 (m, 1H), 3.59-3.31 (m, 4H), 3.29-3.03 (m, 2H), 3.00-2.73 (m, 1H), 2.18-1.67 (m, 9H), 1.60-1.50 (m, 2H), 1.30-0.78 (m, 8H). m/z: ES⁺ [M+H]⁺=585.30.

### Example 2.29: N-ethyl-N-(5-fluoro-2-((4-((1S,5R)-2-((3aR,6aR)-octahydrocyclopenta[c]pyrrole-3a-carbonyl)-2,6-diazabicyclo[3.2.1]octan-6-yl)pyrimidin-5-yl)oxy)phenyl)isobutyramide

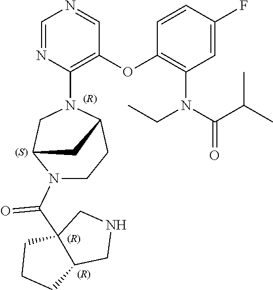

Brown solid, HCl salt (102 mg, 63% over 2 steps).

¹H NMR (400 MHz, DMSO-d₆, 300 K) δ 10.23-9.51 (m, 1H), 9.51-9.04 (m, 1H), 8.83-8.65 (m, 1H), 8.32-7.85 (m, 1H), 7.76-7.03 (m, 3H), 5.26-4.83 (m, 2H), 4.64-4.40 (m, 1H), 4.32-4.15 (m, 1H), 4.10-3.90 (m, 3H), 3.90-3.73 (m, 1H), 3.59-3.53 (m, 1H), 3.48-3.45 (m, 1H), 3.43-3.38 (m, 1H), 3.29-3.12 (m, 2H), 3.08-2.81 (m, 2H), 2.48-2.39 (m, 1H), 2.22-1.67 (m, 9H), 1.22-1.01 (m, 3H), 1.01-0.96 (m, 3H), 0.95-0.78 (m, 2H). m/z: ES⁺ [M+H]⁺=551.30.

### Example 2.30: ((1S,5R)-6-(5-(4-fluoro-2-(2-isopropyl-5-methyl-1H-imidazol-1-yl)phenoxy)pyrimidin-4-yl)-2,6-diazabicyclo[3.2.1]octan-2-yl)((3aR,6aR)-hexahydrocyclopenta[c]pyrrol-3a(1H)-yl)methanone

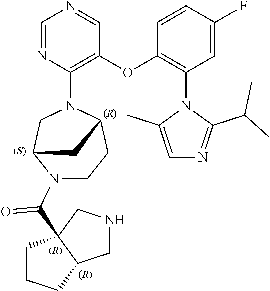

Light yellow solid, HCl salt (65 mg, 50% over 2 steps).

¹H NMR (400 MHz, DMSO-d₆, 300 K) δ 10.05-9.30 (m, 1H), 8.75-8.55 (m, 1H), 8.50-8.20 (m, 1H), 8.00-7.80 (m, 1H), 7.75-7.22 (m, 3H), 4.92-4.50 (m, 1H), 4.12-3.90 (m, 1H), 3.85-3.50 (m, 2H), 3.40-3.25 (m, 2H), 3.22-3.08 (m, 2H), 3.05-2.76 (m, 3H), 2.32-2.15 (m, 3H), 2.10-1.82 (m, 5H), 1.80-1.52 (m, 2H), 1.50-1.42 (m, 3H), 1.40-1.35 (m, 2H), 1.35-1.12 (m, 6H). m/z: ES⁺ [M+H]⁺=560.25.

### Example 2.31: ((1S,5R)-6-(5-(4-fluoro-2-(2-isopropyl-5-methyl-1H-imidazol-1-yl)phenoxy)pyrimidin-4-yl)-2,6-diazabicyclo[3.2.1]octan-2-yl)((S)-2-methylpyrrolidin-2-yl)methanone

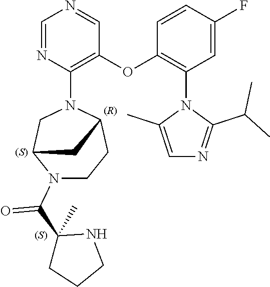

Off-white solid, HCl salt (63 mg, 41% over 2 steps).

¹H NMR (400 MHz, DMSO-d₆, 300 K) δ 10.10-9.80 (m, 1H), 8.92-8.75 (m, 1H), 8.70-8.55 (m, 1H), 8.49-8.32 (m, 1H), 8.05-7.95 (m, 1H), 7.975-7.45 (m, 3H), 4.92-4.50 (m, 1H), 4.12-3.90 (m, 1H), 3.85-3.50 (m, 2H), 3.40-3.15 (m, 2H), 3.12-2.76 (m, 3H), 2.32-2.15 (m, 3H), 2.10-1.82 (m, 3H), 1.80-1.52 (m, 2H), 1.50-1.42 (m, 3H), 1.40-1.35 (m, 3H), 1.35-1.00 (m, 6H). m/z: ES⁺ [M+H]⁺=534.30.

### Example 2.32 and 2.33: atropisomers of 6-(5-fluoro-2-((4-((1S,5R)-2-((S)-2-methylpyrrolidine-2-carbonyl)-2,6-diazabicyclo[3.2.1]octan-6-yl)pyrimidin-5-yl)oxy)phenyl)-5-isopropyl-1-methylpyridin-2(1H)-one

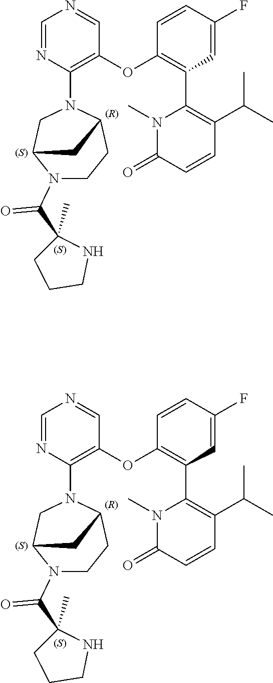

Mixture isolated as yellow solid, HCl salt (110 mg, 56% over 2 steps).

¹H NMR (400 MHz, DMSO-d₆, 300K) δ 10.04 (s, 1H), 8.98-8.63 (m, 2H), 8.30-8.00 (m, 1H), 7.77-7.40 (m, 4H), 6.63-6.45 (m, 1H), 5.22-4.75 (m, 2H), 4.34-3.47 (m, 3H), 3.45-2.70 (m, 6H), 2.43-1.16 (m, 12H), 1.15-0.78 (m, 6H). m/z: ES⁺ [M+H]⁺=561.25.

Separation by chiral HPLC gave two single stable atropisomers:

Peak #1: Rt1=4.32 min, colourless solid (16 mg, 99%).

¹H NMR (400 MHz, DMSO-d₆, 300K) δ 8.32 (s, 1H), 7.82 (s, 1H), 7.54 (d, J=9.6 Hz, 1H), 7.44-7.40 (m, 2H), 7.35-6.90 (m, 1H), 6.50 (d, J=9.6 Hz, 1H), 5.85-5.06 (m, 1H), 4.81-4.65 (m, 1H), 4.58-4.02 (m, 1H), 3.98-3.55 (m, 2H), 3.13 (s, 3H), 2.95-2.80 (m, 2H), 2.70-2.60 (m, 2H), 2.45-2.15 (m, 2H), 1.95-1.80 (m, 1H), 1.75-1.55 (m, 6H), 1.22 (s, 3H), 1.12 (d, J=6.8 Hz, 3H), 0.93 (d, J=6.8 Hz, 3H). m/z: ES⁺ [M+H]⁺=561.30.

Peak #2: Rt2=7.02 min, colourless solid (22 mg, 99%).

¹H NMR (400 MHz, DMSO-d₆, 300K) δ 8.32 (s, 1H), 7.82 (s, 1H), 7.54 (d, J=9.6 Hz, 1H), 7.44-7.40 (m, 2H), 7.35-6.90 (m, 1H), 6.50 (d, J=9.6 Hz, 1H), 5.55-5.06 (m, 1H), 4.81-4.65 (m, 1H), 4.35-4.10 (m, 1H), 3.98-3.55 (m, 2H), 3.13 (s, 3H), 2.95-2.80 (m, 2H), 2.70-2.60 (m, 2H), 2.45-2.15 (m, 2H), 1.95-1.80 (m, 1H), 1.75-1.55 (m, 6H), 1.22 (s, 3H), 1.12 (d, J=6.8 Hz, 3H), 0.93 (d, J=6.8 Hz, 3H). m/z: ES⁺ [M+H]⁺=561.30.

### Example 2.34 ((1S,5R)-6-(5-(4-fluoro-2-(4-isopropyl-1-methyl-1H-1,2,3-triazol-5-yl)phenoxy)pyrimidin-4-yl)-2,6-diazabicyclo[3.2.1]octan-2-yl)((S)-2-methylpyrrolidin-2-yl)methanone

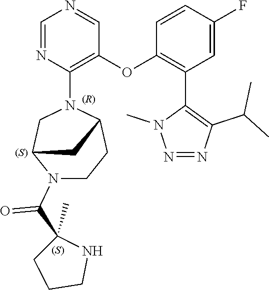

Off-white solid, HCl salt (37 mg, 34% over 2 steps).

¹H NMR (400 MHz, DMSO-d₆, 296 K) δ 10.30-9.79 (m, 1H), 9.30-8.08 (m, 3H), 7.70-7.20 (m, 3H), 5.28-4.09 (m, 2H), 3.87 (s, 3H), 3.73-3.45 (m, 1H), 3.34-3.17 (m, 2H), 3.10-2.72 (m, 2H), 2.32-2.12 (m, 2H), 2.07-1.72 (m, 6H), 1.70-1.45 (m, 5H), 1.29-1.03 (m, 6H). m/z: ES⁺ [M+H]⁺=535.15.

### Examples 2.35 and 2.36: atropisomers of ((1S,5R)-6-(5-(4-fluoro-2-(2-isopropyl-4,5-dimethyl-1H-imidazol-1-yl)phenoxy)pyrimidin-4-yl)-2,6-diazabicyclo[3.2.1]octan-2-yl)((S)-2-methylpyrrolidin-2-yl)methanone

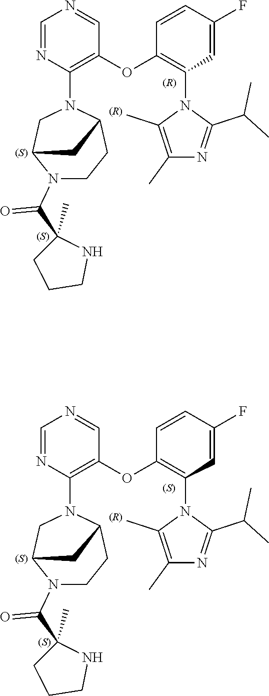

Separation by chiral HPLC gave two single stable atropisomers:

Peak #1: Rt=18.33 min, colourless solid (3.2 mg, 2%).

¹H NMR (400 MHz, DMSO-d₆, 295.6K) δ 8.32 (s, 1H), 7.81 (s, 1H), 7.58-7.49 (m, 1H), 7.45-7.35 (m, 1H), 7.29-7.08 (m, 1H), 5.55-4.91 (m, 1H), 4.79-4.45 (m, 1H), 4.40-3.99 (m, 1H), 3.88-3.45 (m, 2H), 3.30-3.25 (m, 1H), 3.08-2.75 (m, 1H), 2.69-2.52 (m, 2H), 2.38-2.15 (m, 1H), 2.20 (d, J=7.6 Hz, 3H), 1.98-1.80 (m, 5H), 1.78-1.42 (m, 6H), 1.30-1.19 (m, 3H), 1.09-1.01 (m, 6H). m/z: ES⁺ [M+H]⁺=548.30.

Peak #2: Rt=22.35 min, colourless solid (5.4 mg, 3%).

¹H NMR (400 MHz, DMSO-d₆, 296.0 K) δ 8.34 (s, 1H), 7.84 (s, 1H), 7.58-7.49 (m, 1H), 7.48-7.32 (m, 1H), 7.29-7.09 (m, 1H), 5.58-4.91 (m, 1H), 4.76-4.48 (m, 1H), 4.47-3.99 (m, 1H), 3.81-3.42 (m, 2H), 3.30-3.20 (m, 1H), 2.95-2.75 (m, 1H), 2.69-2.55 (m, 2H), 2.30-2.12 (m, 1H), 2.20 (d, J=7.6 Hz, 3H), 1.90-1.80 (m, 5H), 1.75-1.42 (m, 6H), 1.30-1.19 (m, 3H), 1.11-1.01 (m, 6H). m/z: ES⁺ [M+H]⁺=548.35.

### Examples 3.01 to 3.71

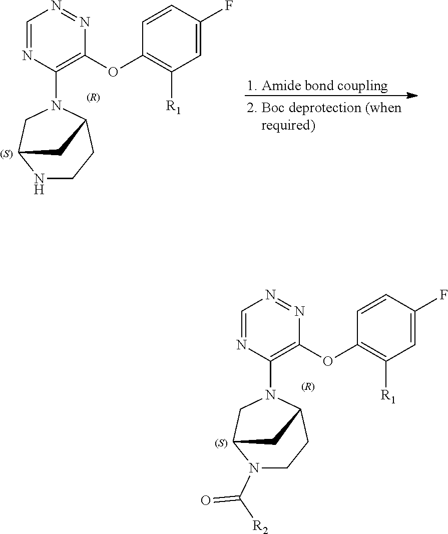

| | | Amide | | Boc | |
|---|---|---|---|---|---|
| | | coupling | Step#1 | deprotect. | Step#2 |
| Ex. | Carboxylic acid reagent | method | purification | procedure | purification |
| 3.01 | dimethylglycine | A1 | Prep-HPLC1; Gradient: | — | — |
| | | | 20% B to 40% B in 8 min; | | |
| | | | Rt = 6.98 min | | |
| 3.02 | dimethylglycine | A1 | Prep-HPLC1; Gradient: | — | — |
| | | | 12% B to 37% B in 7 min; | | |
| | | | Rt = 6.25 min | | |
| 3.03 | (tert-butoxycarbonyl)-L-proline | A1 | RPFC1, 10% to 50% | B2 | Prep-HPLC8; Gradient: |
| | | | MeCN gradient in 10 min | | 29% B to 54% B in 10 |
| | | | | | min; Rt = 10.0 min |
| 3.04 | (S)-1-(tert-butoxycarbonyl)-2- | A1 | RPFC1, 10% to 50% | B1 | Prep-HPLC1; Gradient: |
| | methylpyrrolidine-2-carboxylic | | MeCN gradient in 10 min | | 16% B to 41% B in 7 |
| | acid | | | | min; Rt-6.1 min |
| 3.05 | (S)-1-(tert-butoxycarbonyl)-2- | A1 | RPFC1, 10% to 100% | B2 | Prep-HPLC1; Gradient: |
| | methylpyrrolidine-2-carboxylic | | MeCN gradient in 20 min | | 15% B to 33% B in 8 |
| | acid | | | | min; Rt = 8.03 min |
| 3.06 | (S)-1-(tert-butoxycarbonyl)-2- | A1 | RPFC1, 10% to 80% | B2 | Prep-HPLC1; Gradient: |
| | methylpyrrolidine-2-carboxylic | | MeCN gradient in 20 min | | 13% B to 35% B in 7 |
| | acid | | | | min; Rt = 6.45 min |
| 3.07 | (S)-1-(tert-butoxycarbonyl)-2- | A1 | RPFC4, 10% to 50% | B2 | Prep-HPLC1; Gradient: |
| | ethylpyrrolidine-2-carboxylic | | MeCN gradient in 10 min | | 16% B to 35% B in 7 |
| | acid | | | | min; Rt = 7.72 min |
| 3.08 | (tert-butoxycarbonyl)-L-proline | A1 | RPFC1, 10% to 50% | B2 | RPFC1, 10% to 50% |
| | | | MeCN gradient in 10 min | | MeCN gradient in 10 |
| | | | | | min |
| 3.09 | (S)-1-(tert-butoxycarbonyl)-2- | A1 | RPFC3, 10% to 100% | B1 | Prep-HPLC1; Gradient: |
| | methylpyrrolidine-2-carboxylic | | MeCN gradient in 10 min | | 16% B to 41% B in 7 |
| | acid | | | | min; Rt = 6.35 min |
| 3.10 | (S)-1-(tert-butoxycarbonyl)-2- | A1 | Prep-HPLC1; Gradient: | B2 | Prep-HPLC6; Gradient: |
| | ethylpyrrolidine-2-carboxylic | | 16% B to 41% B in 7 min; | | 4% B to 28% B in 7 min; |
| | acid | | Rt = 6.1 min | | Rt = 6.4 min |
| 3.11 | (1S,3S,5S)-2-(tert- | A1 | RPFC1, 40% to 80% | B1 | Prep-HPLC1; Gradient: |
| | butoxycarbonyl)-2- | | MeCN gradient in 10 min | | 18% B to 43% B in 7 |
| | azabicyclo[3.1.0]hexane-3- | | | | min; Rt = 6.67 min |
| | carboxylic acid | | | | |
| 3.12 | (S)-1-(tert-butoxycarbonyl)-2- | A1 | RPFC1, 40% to 80% | B2 | Prep-HPLC1; Gradient: |
| | ethylpyrrolidine-2-carboxylic | | MeCN gradient in 10 min | | 20% B to 40% B in 7 |
| | acid | | | | min; Rt-6.8 min |
| 3.13 | (1S,3S,5S)-2-(tert- | A1 | RPFC1, 40% to 80% | B2 | Prep-HPLC1; Gradient: |
| | butoxycarbonyl)-2- | | MeCN gradient in 10 min | | 20% B to 40% B in 7 |
| | azabicyclo[3.1.0]hexane-3- | | | | min; Rt-7.0 min |
| | carboxylic acid | | | | |
| 3.14 | (S)-1-(tert-butoxycarbonyl)-2- | A5 | RPFC1, 40% to 80% | B1 | Prep-HPLC1; Gradient: |
| | methylpyrrolidine-2-carboxylic | | MeCN gradient in 10 min | | 16% B to 41% B in 7 |
| | acid | | | | min; Rt = 6.1 min |
| 3.15 | 7-(tert-butoxycarbonyl)-7- | A5 | RPFC1, 10% to 100% | B1 | Prep-HPLC1; Gradient: |
| | azabicyclo[2.2.1]heptane-1- | | MeCN gradient in 10 min | | 14% B to 39% B in 7 |
| | carboxylic acid | | | | min; Rt = 6.65 min |
| 3.16 | (S)-1-(tert-butoxycarbonyl)-2- | A1 | RPFC2, 10% to 50% | B2 | Prep-HPLC1; Gradient: |
| | ethylpyrrolidine-2-carboxylic | | MeCN gradient in 10 min | | 23% B to 48% B in 8 |
| | acid | | | | min; Rt = 7.28 min |
| 3.17 | (S)-1-(tert-butoxycarbonyl)-2- | A1 | RPFC1, 10% to 100% | B1 | Prep-HPLC1; Gradient: |
| | ethylpyrrolidine-2-carboxylic | | MeCN gradient in 25 min | | 16% B to 41% B in 8 |
| | acid | | | | min; Rt = 7.43 min |
| 3.18 | tetrahydro-1H-pyrrolizine- | A1 | Prep-HPLC1; Gradient: | | |
| | 7a(5H)-carboxylic acid | | 21% B to 38% B in 7 min; | — | — |
| | | | Rt = 6.1 min | | |
| 3.19 | (S)-1-(tert-butoxycarbonyl)-2- | A1 | RPFC1, 10% to 100% | B2 | Prep-HPLC6; Gradient: |
| | methylpyrrolidine-2-carboxylic | | MeCN gradient in 10 min | | 0.5% B to 15% B in 7 |
| | acid | | | | min; Rt = 3.9 min |
| 3.20 | (S)-1-(tert-butoxycarbonyl)-2- | A1 | RPFC3, 0% to 100% | B2 | Prep-HPLC21; Gradient: |
| | ethylpyrrolidine-2-carboxylic | | MeCN gradient in 30 min | | 6% B to 23% B in 7 min; |
| | acid | | | | Rt = 5.03 min |
| 3.21 | (S)-1-(tert-butoxycarbonyl)-2- | A1 | RPFC1, 30% to 70% | B1 | Prep-HPLC1; Gradient: |
| | methylpyrrolidine-2-carboxylic | | MeCN gradient in 10 min | | 16% B to 41% B in 7 |
| | acid | | | | min; Rt = 6.1 min |
| 3.27 | (1S,2S,5R)-3-(tert- | A1 | RPFC1, 40% to 80% | B2 | Prep-HPLC6; Gradient: |
| | butoxycarbonyl)-3- | | MeCN gradient in 10 min | | 2% B to 19% B in 7 min; |
| | azabicyclo[3.1.0]hexane-2- | | | | Rt = 6.3 min |
| | carboxylic acid | | | | |
| 3.23 | rac-(3aR,6aR)-2-(tert- | A1 | RPFC1, 10% to 100% | B2 | Prep-HPLC6; Gradient: |
| | butoxycarbonyl)hexahydro- | | MeCN gradient in 25 min | | 2% B to 14% B in 7 min; |
| | cyclopenta[c]pyrrole-3a(1H)- | | | | Rt = 5.9 min. |
| | carboxylic acid | | | | CHIRALPAK IM; |
| | | | | | A: Hex(0.2% IPamine), |
| | | | | | B: EtOAc:DCM = 1:1; |
| | | | | | gradient: isocratic 55% |
| | | | | | B; Rt = 7.34 min |
| 3.24 | rac-(3aR,6aR)-2-(tert- | A1 | RPFC1, 10% to 100% | B2 | Prep-HPLC6; Gradient: |
| | butoxycarbonyl)hexahydro- | | MeCN gradient in 25 min | | 2% B to 14% B in 7 min; |
| | cyclopenta[c]pyrrole-3a(1H)- | | | | Rt = 5.9 min. |
| | carboxylic acid | | | | CHIRALPAK IM; |
| | | | | | A: Hex(0.2% IPamine), |
| | | | | | B: EtOAc:DCM = 1:1; |
| | | | | | gradient: isocratic 55% |
| | | | | | B; Rt = 13.41 min |
| 3.25 | rac-(3aR,6aR)-2-(tert- | A1 | RPFC1, 10% to 50% | B2 | Prep-HPLC6; Gradient: |
| | butoxycarbonyl)- | | MeCN gradient in 10 min | | 5% B to 20% B in 7 min; |
| | hexahydrocyclopenta[c]pyrrole- | | | | Rt = 6.5 min. |
| | 3a(1H)-carboxylic acid | | | | CHIRALPAK IK; |
| | | | | | A: Hex(0.2% IPAmine), |
| | | | | | B: EtOH:DCM = 1:1; |
| | | | | | gradient: isocratic 50% |
| | | | | | B; Rt = 6.38 min |
| 3.26 | rac-(3aR,6aR)-2-(tert- | A1 | RPFC1, 10% to 50% | B2 | Prep-HPLC6; Gradient: |
| | butoxycarbonyl)- | | MeCN gradient in 10 min | | 5% B to 20% B in 7 min; |
| | hexahydrocyclopenta[c]pyrrole- | | | | Rt = 6.5 min. |
| | 3a(1H)-carboxylic acid | | | | CHIRALPAK IK; |
| | | | | | A: Hex(0.2% IPAmine), |
| | | | | | B: EtOH:DCM = 1:1; |
| | | | | | gradient: isocratic 50% |
| | | | | | B; Rt = 11.33 min |
| 3.27 | rac-(3aR,6aR)-2-(tert- | A1 | RPFC1, 10% to 100% | B2 | Prep-HPLC6; Gradient: |
| | butoxycarbonyl)hexahydro- | | MeCN gradient in 20 min | | 7% B to 29% B in 7 min; |
| | cyclopenta[c]pyrrole-3a(1H)- | | | | Rt = 5.13 min. |
| | carboxylic acid | | | | CHIRALPAK IG; |
| | | | | | A: Hex(0.5% 2M |
| | | | | | NH₃—MeOH), B: |
| | | | | | EtOH:DCM = 1:1; |
| | | | | | gradient: isocratic 45% |
| | | | | | B; Rt = 8.19 min |
| 3.28 | rac-(3aR,6aR)-2-(tert- | A1 | RPFC1, 10% to 100% | B2 | Prep-HPLC6; Gradient: |
| | butoxycarbonyl)hexahydro- | | MeCN gradient in 20 min | | 7% B to 29% B in 7 min; |
| | cyclopenta[c]pyrrole-3a(1H)- | | | | Rt = 5.13 min. |
| | carboxylic acid | | | | CHIRALPAK IG; |
| | | | | | A: Hex(0.5% 2M |
| | | | | | NH₃]MeOH), B: |
| | | | | | EtOH:DCM = 1:1; |
| | | | | | gradient: isocratic 45% |
| | | | | | B; Rt = 11.8 min |
| 3.29 | (1S,2S,5R)-3-(tert- | A1 | RPFC1, 10% to 100% | B2 | Prep-HPLC6; Gradient: |
| | butoxycarbonyl)-3- | | MeCN gradient in 20 min | | 6% B to 28% B in 7 min; |
| | azabicyclo[3.1.0]hexane-2- | | | | Rt = 5.15 min. |
| | carboxylic acid | | | | |
| 3.30 | 2-((R)-1-(tert- | A1 | Prep-TLC | B2 | CHIRALPAK IK; |
| | butoxycarbonyl)pyrrolidin-3- | | PE/EtOAc (1:1) | | A: Hex(0.2% DEA), B: |
| | yl)propanoic acid | | | | EtOH:DCM = 1:1; |
| | | | | | gradient: isocratic 60% |
| | | | | | B; Rt = 5.09 min |
| 3.31 | 2-((R)-1-(tert- | A1 | Prep-TLC | B2 | CHIRALPAK IK; |
| | butoxycarbonyl)pyrrolidin-3- | | PE/EtOAc (1:1) | | A: Hex(0.2% DEA), B: |
| | yl)propanoic acid | | | | EtOH:DCM = 1:1; |
| | | | | | gradient: isocratic 60% |
| | | | | | B; Rt = 8.67 min |
| 3.32 | hexahydropyrrolizine-7a- | A1 | Prep-HPLC1; Gradient: | — | — |
| | carboxylic acid | | 15% B to 35% B in 7 min | | |
| | | | Rt = 6.78 min | | |
| 3.33 | 7-(tert-butoxycarbonyl)-7- | A1 | RPFC1, 60% to 800% | B1 | Prep-HPLC1; Gradient: |
| | azabicyclo[2.2.1]heptane-1- | | MeCN gradient in 10 min | | 19% B to 44% B in 7 |
| | carboxylic acid | | | | min; Rt = 6.27 min |
| 3.34 | rac-(3aR,6aR)-2-(tert- | A1 | RPFC1, 10% to 100% | B2 | Prep-HPLC20; Gradient: |
| | butoxycarbonyl)hexahydro- | | MeCN gradient in 20 min | | 24% B to 43% B in 7 |
| | cyclopenta[c]pyrrole-3a(1H)- | | | | min; Rt = 7.0 min. |
| | carboxylic acid | | | | CHIRALPAK IM; |
| | | | | | A: Hex(0.2% IPAmine), |
| | | | | | B: EtOH:DCM = 1:1; |
| | | | | | gradient: isocratic 5% B; |
| | | | | | Rt = 8.24 min |
| 3.35 | rac-(3aR,6aR)-2-(tert- | A1 | RPFC1, 10% to 100% | B2 | Prep-HPLC20; Gradient: |
| | butoxycarbonyl)hexahydro- | | MeCN gradient in 20 min | | 24% B to 43% B in 7 |
| | cyclopenta[c]pyrrole-3a(1H)- | | | | min; Rt = 7.0 min. |
| | carboxylic acid | | | | CHIRALPAK IM; |
| | | | | | A: Hex(0.2% IPAmine), |
| | | | | | B: EtOH:DCM = 1:1; |
| | | | | | gradient: isocratic 5% B; |
| | | | | | Rt = 20.25 min |
| 3.36 | (S)-1-(tert-butoxycarbonyl)-2- | A1 | RPFC1, 10% to 90% | B1 | Prep-HPLC1; Gradient: |
| | ethylpyrrolidine-2-carboxylic | | MeCN gradient in 10 min | | 26% B to 41% B in 7 |
| | acid | | | | min; Rt = 7.12 min |
| 3.37 | (difluoromethoxy)acetic acid | A1 | Prep-HPLC6; Gradient: | | |
| | | | 2% B to 30% B in 8 min; | — | — |
| | | | Rt = 7.52 min | | |
| 3.38 | (S)-1-(tert-butoxycarbonyl)-2- | A1 | RPFC1, 0% to 100% | B2 | Prep-HPLC1; Gradient: |
| | ethylpyrrolidine-2-carboxylic | | MeCN gradient in 30 min | | 15% B to 40% B in 8 |
| | Jacic | | | | min; Rt = 7.8 min |
| 3.39 | (rac) 2-(tert- | A1 | RPFC1, 10% to 100% | B2 | — |
| | butoxycarbonyl)hexahydro- | | MeCN gradient in 20 min | | |
| | cyclopenta[c]pyrrole-3a(1H)- | | | | |
| | carboxylic acid | | | | |
| 3.40 | (3aR,6aR)-2-(tert- | A1 | RPFC1, 10% to 80% | B2 | Prep-HPLC6, Gradient |
| | butoxycarbonyl)hexahydro- | | MeCN gradient in 30 min | | 7% B to 37% B in 7 min; |
| | cyclopenta[c]pyrrole-3a(1H)- | | | | Rt = 6.50 min |
| | carboxylic acid | | | | |
| 3.41 | (rac) 2-(tert- | A1 | RPFC1, 10% to 100% | B2 | CHIRALPAKIM; |
| | butoxycarbonyl)hexahydro- | | MeCN gradient in 20 min | | isocratic A: Hex(0.2% |
| | cyclopenta[c]pyrrole-3a(1H)- | | | | IPAmine), B: |
| | carboxylic acid | | | | EtOH:DCM = 1:1; 60% B; |
| | | | | | Rt = 5.13 |
| 3.42 | (rac) 2-(tert- | A1 | RPFC1, 10% to 100% | B2 | CHIRALPAKIM; |
| | butoxycarbonyl)hexahydro- | | MeCN gradient in 20 min | | isocratic A: Hex(0.2% |
| | cyclopenta[c]pyrrole-3a(1H)- | | | | IPAmine), B: |
| | carboxylic acid | | | | EtOH:DCM = 1:1; 60% B; |
| | | | | | Rt = 9.87 |
| 3.43 | (S)-1-(tert-butoxycarbonyl)-2- | A1 | RPFC1, 10% to 90% | B2 | Prep-HPLC1; Gradient |
| | methylpyrrolidine-2-carboxylic | | MeCN gradient in 15 min | | 20% B to 32% B in 7 |
| | acid | | | | min; Rt = 6.98 min |
| 3.44 | (3aR,6aR)-2-(tert- | A1 | RPFC1, 0% to 80% MeCN | B2 | Prep-HPLC1; Gradient |
| | butoxycarbonyl)hexahydro- | | gradient in 30 min | | 13% B to 43% B in 8 |
| | cyclopenta[c]pyrrole-3a(1H)- | | | | min; Rt = 7.7 min |
| | carboxylic acid | | | | |
| 3.45 | (S)-1-(tert-butoxycarbonyl)-2- | A1 | Prep-TLC (DCM/MeOH | B2 | Trituration with EtOAc |
| | ethylpyrrolidine-2-carboxylic | | 10:1) | | |
| | acid | | | | |
| 3.46 | tetrahydro-1H-pyrrolizine- | A1 | Prep-HPLC1; Gradient | — | — |
| | 7a(5H)-carboxylic acid | | 14% B to 36% B in 7 min | | |
| | | | Rt = 6.42 min | | |
| 3.47 | (3aR,6aR)-2-(tert- | A1 | RPFC1, 10% to 100% | B1 | Prep-HPLC1; Gradient |
| | butoxycarbonyl)hexahydro- | | MeCN gradient in 25 min | | 17% B to 42% B in 7 |
| | cyclopenta[c]pyrrole-3a(1H)- | | | | min; Rt = 6.47 min |
| | carboxylic acid | | | | |
| 3.48 | (3aR,6aR)-2-(tert- | A1 | RPFC1, 10% to 100% | B2 | Prep-HPLC1; Gradient |
| | butoxycarbonyl)hexahydro- | | MeCN gradient in 20 min | | 5% B to 22% B in 8 min; |
| | cyclopenta[c]pyrrole-3a(1H)- | | | | Rt = 6.3 min |
| | carboxylic acid | | | | |
| 3.49 | (3aR,6aR)-2-(tert- | A1 | RPFC4, 70% to 80% | B1 | Prep-HPLC1; Gradient |
| | butoxycarbonyl)hexahydro- | | MeCN gradient in 10 min | | 25% B to 35% B in 7 |
| | cyclopenta[c]pyrrole-3a(1H)- | | | | min; Rt = 6.9 min |
| | carboxylic acid | | | | |
| 3.50 | (3aR,6aR)-2-(tert- | A1 | RPFC1, 10% to 100% | B2 | Prep-HPLC1; Gradient |
| | butoxycarbonyl)hexahydro- | | MeCN gradient in 20 min | | 12% B to 42% B in 8 |
| | cyclopenta[c]pyrrole-3a(1H)- | | | | min; Rt = 7.67 min |
| | carboxylic acid | | | | |
| 3.51 | (3aR,6aR)-2-(tert- | A1 | Prep-TLC (DCM/MeOH | B2 | Trituration with EtOAc |
| | butoxycarbonyl)hexahydro- | | 10:1) | | |
| | cyclopenta[c]pyrrole-3a(1H)- | | | | |
| | carboxylic acid | | | | |
| 3.52 | (S)-1-(tert-butoxycarbonyl)-2- | A1 | RPFC1, 40% to 80% | B2 | Prep-HPLC1, Gradient |
| | methylpyrrolidine-2-carboxylic | | MeCN in 15 min | | 19% B to 38% B in 7 |
| | acid | | | | min; Rt = 6.65 min |
| 3.53 | (3aR,6aR)-2-(tert- | A1 | RPFC1, 50% to 60% | B2 | Trituration with EtOAc |
| | butoxycarbonyl)hexahydro- | | MeCN gradient in 10 min | | |
| | cyclopenta[c]pyrrole-3a(1H)- | | | | |
| | carboxylic acid | | | | |
| 3.54 | (3aR,6aR)-2-(tert- | A1 | RPFC1, 0% to 100% | B2 | Trituration with EtOAc |
| | butoxycarbonyl)hexahydro- | | MeCN gradient in 10 min | | |
| | cyclopenta[c]pyrrole-3a(1H)- | | | | |
| | carboxylic acid | | | | |
| 3.55 | tetrahydro-1H-pyrrolizine- | A1 | Prep-HPLC2; Gradient | — | — |
| | 7a(5H)-carboxylic acid | | 36% B to 61% B in 10 min; | | |
| | | | Rt = 7.88 min | | |
| 3.56 | rac-(3aR,6aS)-2-(tert- | A1 | RPFC1, 10% to 100% | B1 | Prep-HPLC1, Gradient |
| | butoxycarbonyl)-6a-fluoro- | | MeCN gradient in 20 min | | 19% B to49% B in 8 |
| | hexahydrocyclopenta[c]pyr- | | | | min; Rt = 7.52 min |
| | role-3a(1H)-carboxylic acid | | | | |
| 3.57 | (3aR,6aR)-2-(tert- | A1 | RPFC1, 10% to 100% | B1 | 1. Prep-HPLC24, |
| | butoxycarbonyl)hexahydro- | | MeCN gradient in 20 min | | Gradient 22% B to 39% |
| | cyclopenta[c]pyrrole-3a(1H)- | | | | B in 7 min; Rt = 7.42 min. |
| | carboxylic acid | | | | 2. CHIRAL ART |
| | | | | | isocratic Hex(0.2%) |
| | | | | | IPAmine): (EtOH:DCM = |
| | | | | | 1:1) = 45:55; |
| | | | | | Rt = 10.73 min |
| 3.58 | (3aR,6aR)-2-(tert- | A1 | RPFC1, 10% to 100% | B1 | 1. Prep-HPLC24, |
| | butoxycarbonyl)hexahydro- | | MeCN gradient in 20 min | | Gradient 22% B to 39% |
| | cyclopenta[c]pyrrole-3a(1H)- | | | | B in 7 min; Rt = 7.42 min. |
| | carboxylic acid | | | | 2. CHIRAL ART |
| | | | | | isocratic Hex(0.2% |
| | | | | | IPAmine): (EtOH:DCM = |
| | | | | | 1:1) = 45:55; |
| | | | | | Rt = 21.37 min |
| 3.59 | (3aR,6aR)-2- | A1 | RPFC1; Gradient: 10% B | — | — |
| | methylhexahydrocyclopenta[c]pyr- | | to 90% B in 25 min; | | |
| | role-3a(1H)-carboxylic acid | | followed by Prep-HPLC6; | | |
| | | | Gradient 5% B to 28% B in | | |
| | | | 7 min; Rt = 6.5 min | | |
| 3.60 | (S)-1-(tert-butoxycarbonyl)-2- | A1 | RPFC1, 10% to 100% | B1 | Prep-HPLC11, Gradient |
| | ethylpyrrolidine-2-carboxylic | | MeCN gradient in 20 min | | 3% B to 27% B in 12 |
| | acid | | | | min; Rt = 11.77 min |
| 3.61 | (rac) 2-(tert- | A1 | RPFC1, 0% to 100% | B2 | CHIRALPAKIK; |
| | butoxycarbonyl)hexahydro- | | MeCN gradient in 30 min | | isocratic A: Hex(0.2% |
| | cyclopenta[c]pyrrole-3a(1H)- | | | | IPAmine), B: |
| | carboxylic acid | | | | EtOH:DCM = 1:1; 50% B; |
| | | | | | Rt = 6.33 |
| 3.62 | (rac) 2-(tert- | A1 | RPFC1, 0% to 100% | B2 | CHIRALPAKIK; |
| | butoxycarbonyl)hexahydro- | | MeCN gradient in 30 min | | isocratic A: Hex(0.2% |
| | cyclopenta[c]pyrrole-3a(1H)- | | | | IPAmine), B: |
| | carboxylic acid | | | | EtOH:DCM = 1:1; 50% B; |
| | | | | | Rt = 8.56 |
| 3.63 | (S)-1-(tert-butoxycarbonyl)-2- | A1 | RPFC1, 10% to 50% | B2 | Prep-HPLC1; Gradient: |
| | ethylpyrrolidine-2-carboxylic | | MeCN gradient in 10 min | | 17% B to 47% B in 8 |
| | acid | | | | min; Rt = 6.63 min |
| 3.64 | (3aR,6aR)-2-(tert- | A1 | RPFC1, 10% to 100% | B2 | Prep-HPLC6; Gradient: |
| | butoxycarbonyl)hexahydro- | | MeCN gradient in 20 min | | 3% B to 33% B in 7 min; |
| | cyclopenta[c]pyrrole-3a(1H)- | | | | Rt = 6.53 min |
| | carboxylic acid | | | | |
| 3.65 | (3aR,6aR)-2-(tert- | A1 | RPFC1, 10% to 70% | B2 | Prep = HPLC6; Gradient |
| | butoxycarbonyl)hexahydro- | | MeCN gradient in 20 min | | 4% B to 32% B in 7 min; |
| | cyclopenta[c]pyrrole-3a(1H)- | | | | Rt = 6.4 min |
| | carboxylic acid | | | | |
| 3.66 | (3aR,6aR)-2-(tert- | A1 | RPFC1, 10% to 100% | B2 | Prep-HPLC6; Gradient |
| | butoxycarbonyl)hexahydro- | | MeCN gradient in 20 min | | 6% B to 36% B in 7 min; |
| | cyclopenta[c]pyrrole-3a(1H)- | | | | Rt = 6.3 min |
| | carboxylic acid | | | | |
| 3.67 | (3aR,6aR)-2-methylhexahydro- | A1 | Prep-HPLC1; Gradient: | | |
| | cyclopenta[c]pyrrole- | | 29% B to 43% B in 7 min;- | — | — |
| | 3a(1H)-carboxylic acid | | Rt = 7.52 min | | |
| 3.68 | (S)-1-(tert-butoxycarbonyl)-2- | A1 | RPFC1, 10% to 100% | B2 | 1. Prep-HPLC1; |
| | ethylpyrrolidine-2-carboxylic | | MeCN gradient in 20 min | | Gradient: 2% B to 15% B |
| | acid | | | | in 7 min; Rt = 6.7 min |
| | | | | | 2. CHIRALPAK ID, |
| | | | | | isocratic Hex(0.2% |
| | | | | | IPAmine): (EtOH:DCM = |
| | | | | | 1:1) = 50:50; |
| | | | | | Rt = 6.35 min |
| 3.69 | (S)-1-(tert-butoxycarbonyl)-2- | A1 | RPFC1, 10% to 100% | B2 | 1. Prep-HPLC1; |
| | ethylpyrrolidine-2-carboxylic | | MeCN gradient in 20 min | | Gradient: 2% B to 15% B |
| | acid | | | | in 7 min; Rt = 6.7 min |
| | | | | | 2. CHIRALPAK ID, |
| | | | | | isocratic Hex(0.2% |
| | | | | | IPAmine): (EtOH:DCM = |
| | | | | | 1:1) = 50:50; |
| | | | | | Rt = 7.83 min |
| 3.70 | (3aR,6aR)-2-(tert- | A1 | RPFC1, 40% to 80% h | [B2 | 1. Prep-HPLC16, |
| | butoxycarbonyl)hexahydro- | | MeCN gradient in 20 min | | Gradient 2% B to 17% B |
| | cyclopenta[c]pyrrole-3a(1H)- | | | | in 7 min; Rt = 6.10 min. |
| | carboxylic acid | | | | 2. CHIRALPAK IK, |
| | | | | | isocratic Hex(0.2% |
| | | | | | IPAmine): (IPA:DCM = |
| | | | | | 1:1) = 50:50; |
| | | | | | Rt = 7.93 min |
| 3.71 | (3aR,6aR)-2-(tert- | A1 | RPFC1, 40% to 80% | B2 | 1. Prep-HPLC16, |
| | butoxycarbonyl)hexahydro- | | MeCN gradient in 20 min | | Gradient 2% B to 17% B |
| | cyclopenta[c]pyrrole-3a(1H)- | | | | in 7 min; Rt = 6.10 min. |
| | carboxylic acid | | | | 2. CHIRALPAK IK, |
| | | | | | isocratic Hex(0.2% |
| | | | | | IPAmine): (IPA:DCM = |
| | | | | | 1:1) = 50:50; |
| | | | | | Rt = 14.33 min |
| 3.72 | (1S,3S,5S)-2-(tert- | A1 | RPFC1, 10% to 50% | B2 | RPFC1, 10% to 50% |
| | butoxycarbonyl)-2- | | MeCN gradient in 10 min | | MeCN gradient in 10 |
| | azabicyclo[3.1.0]hexane-3- | | | | min |
| | carboxylic acid | | | | |
| 3.73 | (1S,2RS,5R)-3-(tert- | A1 | RPFC1, 10% to 100% | B2 | Prep-HPLC16, Gradient |
| | butoxycarbonyl)-2-methyl-3- | | MeCN gradient in 20 min | | 2% B to 18% B in 7 |
| | azabicyclo[3.1.0]hexane-2- | | | | min; Rt = 6.2 min |
| | carboxylic acid | | | | |
| 3.74 | (1R,2RS,5S)-3-(tert- | A1 | RPFC1, 10% to 80% | B2 | Prep-HPLC16, Gradient |
| | butoxycarbonyl)-2-methyl-3- | | MeCN gradient in 20 min | | 1% B to 14% B in 7 min; |
| | azabicyclo[3.1.0]hexane-2- | | | | Rt = 6.2 min |
| | carboxylic acid | | | | |
| 3.75 | rac-(1R,5R)-3-(tert-butoxy- | A1 | — | B1 | Prep-HPLC1, Gradient |
| | carbonyl)-3- | | | | 23% B to 41% B in 7 |
| | azabicyclo[3.1.0]hexane-1- | | | | min; Rt = 7.55 min |
| | carboxylic acid | | | | |
| 3.76 | hexahydropyrrolizine-7a- | A1 | RPFC2, 10% to 50% | — | — |
| | carboxylic acid | | MeCN gradient in 10 min | | |
| 3.77 | (S)-1-(tert-butoxycarbonyl)-2- | A1 | RPFC1, 50% to 70% | B2 | CHIRALPAKIK |
| | ethylpyrrolidine-2-carboxylic | | MeCN gradient in 10 min | | isocratic Hex(0.2% |
| | acid | | | | IPAmine): (IPA:DCM = |
| | | | | | 1:1) = 60:40; |
| | | | | | Rt = 10.02 min |
| 3.78 | (S)-1-(tert-butoxycarbonyl)-2- | A1 | RPFC1, 50% to 70% | B2 | CHIRALPAKIK |
| | ethylpyrrolidine-2-carboxylic | | MeCN gradient in 10 min | | isocratic Hex(0.2% |
| | acid | | | | IPAmine): (IPA:DCM = |
| | | | | | 1:1) = 60:40; |
| | | | | | Rt = 16.72 min |
| 3.79 | (S)-1-(tert-butoxycarbonyl)-2- | A5 | RPFC1, 30% to 80% | B2 | 1. Prep-HPLC16, |
| | methylpyrrolidine-2-carboxylic | | MeCN gradient in 10 min | | Gradient 5% B to 25% B |
| | acid | | | | in 7 min; Rt = 6.10 min. |
| | | | | | 2. CHIRALPAK IH, |
| | | | | | isocratic Hex(0.2% |
| | | | | | IPAmine): (IPA:DCM = |
| | | | | | 1:1) = 65:35; |
| | | | | | Rt = 3.94 min |
| 3.80 | (S)-1-(tert-butoxycarbonyl)-2- | A5 | RPFC1, 30% to 80% | B2 | 1. Prep-HPLC16, |
| | methylpyrrolidine-2-carboxylic | | MeCN gradient in 10 min | | Gradient 5% B to 25% B |
| | acid | | | | in 7 min; Rt = 6.10 min. |
| | | | | | 2. CHIRALPAK IH, |
| | | | | | isocratic Hex(0.2% |
| | | | | | IPAmine): (IPA:DCM = |
| | | | | | 1:1) = 65:35; |
| | | | | | Rt = 6.27 min |
| 3.81 | (3aR,6aR)-2-(tert- | A1 | Silica gel chromatography, | B2 | Prep-HPLC1, Gradient |
| | butoxycarbonyl)hexahydro- | | PE/EtOAc (1:1) | | 17% B to 42% B in 7 |
| | cyclopenta[c]pyrrole-3a(1H)- | | | | min; Rt = 6.37 min |
| | carboxylic acid | | | | |
| 3.82 | (3aR,6aR)-2-(tert- | A1 | RPFC1, 30% to 80% | B2 | Prep-HPLC6, Gradient |
| | butoxycarbonyl)hexahydro- | | MeCN gradient in 20 min | | 5% B to 24% B in 7 min; |
| | cyclopenta[c]pyrrole-3a(1H)- | | | | Rt = 7.32 min |
| | carboxylic acid | | | | |
| 3.83 | (3aR,6aR)-2-(tert- | A1 | RPFC1, 10% to 100% | B2 | Precipitation from |
| | butoxycarbonyl)hexahydro- | | MeCN gradient in 20 min | | EtOAc |
| | cyclopenta[c]pyrrole-3a(1H)- | | | | |
| | carboxylic acid | | | | |
| 3.84 | rac-(3aR,6aS)-2-(tert- | A1 | RPFC1, 30% to 90% | B2 | Precipitation from |
| | butoxycarbonyl)-6a-fluoro- | | MeCN gradient in 10 min | | EtOAc |
| | hexahydrocyclopenta[c]pyr- | | | | |
| | role-3a(1H)-carboxylic acid | | | | |
| 3.85 | (3aR,6aR)-2-(tert- | A1 | RPFC1, 20% to 70% | B1 | Prep-HPLC2, Gradient |
| | butoxycarbonyl)hexahydro- | | MeCN gradient in 10 min | | 51% B to 76% B in 12 |
| | cyclopenta[c]pyrrole-3a(1H)- | | | | min; Rt = 10.69 min |
| | carboxylic acid | | | | |
| 3.86 | (1S,2S,5R)-3-(tert- | A1 | RPFC4, 10% to 50% | B1 | Prep-HPLC1, Gradient |
| | butoxycarbonyl)-3- | | MeCN gradient in 10 min | | 13% B to 38% B in 7 |
| | azabicyclo[3.1.0]hexane-2- | | | | min; Rt = 6.85 min |
| | carboxylic acid | | | | |
| 3.87 | (3aR,6aR)-2-(tert- | A1 | RPFC1, 10% to 100% | B1 | Prep-HPLC1, Gradient |
| | butoxycarbonyl)hexahydro- | | MeCN gradient in 20 min | | 24% B to 54% B in 8 |
| | cyclopenta[c]pyrrole-3a(1H)- | | | | min; Rt = 7.97 min |
| | carboxylic acid | | | | |
| 3.88 | (S)-1-(tert-butoxycarbonyl)-2- | A1 | RPFC1, 10% to 100% | B1 | Prep-HPLC1, Gradient |
| | ethylpyrrolidine-2-carboxylic | | MeCN gradient in 20 min | | 29% B to 51% B in 7 |
| | acid | | | | min; Rt = 6.8 min |

### Example 3.01: 1-((1S,5R)-6-(6-(2-(4-cyclopropylpyrimidin-5-yl)-4-fluorophenoxy)-1,2,4-triazin-5-yl)-2,6-diazabicyclo[3.2.1]octan-2-yl)-2-(dimethylamino)ethan-1-one

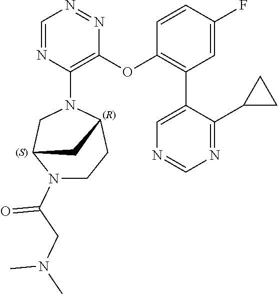

Colourless solid (6 mg, 6%).

¹H NMR (400 MHz, DMSO-d₆, 300 K) δ 8.80-8.36 (m, 1H), 7.85-7.25 (m, 3H), 6.92-7.01 (m, 1H), 6.89-6.60 (m, 1H), 5.30-4.57 (m, 2H), 4.36-3.50 (m, 3H), 3.25-3.05 (m, 2H), 2.87-2.55 (m, 1H), 2.17-1.99 (m, 6H), 1.98-1.45 (m, 5H), 0.96-0.58 (m, 4H). m/z: ES⁺ [M+H]⁺=493.35.

### Example 3.02: 1-((1S,5R)-6-(6-(2-(2-Cyclopropyl-1H-imidazol-1-yl)-4-fluorophenoxy)-1,2,4-triazin-5-yl)-2,6-diazabicyclo[3.2.1]octan-2-yl)-2-(dimethylamino)ethan-1-one

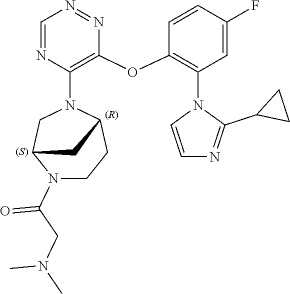

Colourless solid (56 mg, 39%).

¹H NMR (400 MHz, DMSO-d₆, 300 K) δ 8.80-8.36 (m, 1H), 7.85-7.25 (m, 3H), 6.92-7.01 (m, 1H), 6.89-6.60 (m, 1H), 5.30-4.57 (m, 2H), 4.36-3.50 (m, 3H), 3.25-3.05 (m, 2H), 2.87-2.55 (m, 1H), 2.17-1.99 (m, 6H), 1.98-1.45 (m, 5H), 0.96-0.58 (m, 4H). m/z: ES⁺ [M+H]⁺=493.35.

### Example 3.03: (1S,5R)-2-(L-prolyl)-6-(6-(2-(4,6-dimethylpyrimidin-5-yl)-4-fluorophenoxy)-1,2,4-triazin-5-yl)-2,6-diazabicyclo[3.2.1]octane

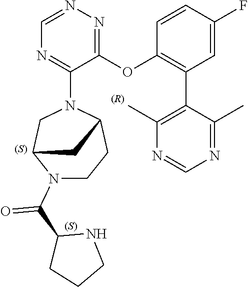

Colourless solid (16 mg, 10% over 2 steps).

¹H NMR (400 MHz, DMSO-d₆, 300 K) δ 8.90-8.74 (m, 1H), 8.55-8.35 (m, 1H), 7.63-7.49 (m, 1H), 7.47-7.35 (m, 2H), 5.20-5.07 (m, 1H), 4.80-4.58 (m, 2H), 4.25-4.07 (m, 1H), 3.94-3.64 (m, 2H), 3.64-3.48 (m, 1H), 3.14-2.95 (m, 2H), 2.93-2.79 (m, 1H), 2.79-2.62 (m, 1H), 2.27-2.00 (m, 6H), 2.01-1.51 (m, 6H), 1.51-1.19 (m, 1H). m/z: ES⁺ [M+H]⁺=505.20.

### Example 3.04: 5-Fluoro-N,N-diisopropyl-2-((5-((1S,5R)-2-((S)-2-methylpyrrolidine-2-carbonyl)-2,6-diazabicyclo[3.2.1]octan-6-yl)-1,2,4-triazin-6-yl)oxy)benzamide

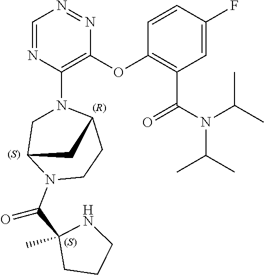

Colourless solid, formic acid salt (27 mg, 11% over 2 steps).

¹H NMR (400 MHz, DMSO-d6, 300 K) δ 8.60-8.47 (m, 1H), 8.31-8.11 (m, 1H), 7.75-7.22 (m, 3H), 5.33-4.71 (m, 2H), 4.39-3.79 (m, 3H), 3.79-3.50 (m, 2H), 3.50-3.12 (m, 2H), 3.09-2.71 (m, 3H), 2.37-1.62 (m, 8H), 1.50-1.31 (m, 5H), 1.30-0.99 (m, 6H), 0.88-0.72 (m, 1H), 0.72-0.50 (m, 2H). m/z: ES⁺ [M+H]⁺=540.45.

### Example 3.05: ((1S,5R)-6-(6-(2-(2-cyclopropyl-1H-imidazol-1-yl)-4-fluorophenoxy)-1,2,4-triazin-5-yl)-2,6-diazabicyclo[3.2.1]octan-2-yl)((S)-2-methylpyrrolidin-2-yl)methanone

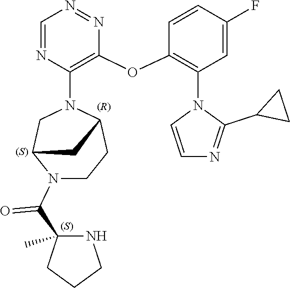

Off-white solid (20 mg, 7% over 2 steps).

¹H NMR (400 MHz, DMSO-d₆, 300 K) δ 8.65-8.50 (m, 1H), 7.72-7.60 (m, 1H), 7.53-7.40 (m, 2H), 7.05-6.95 (m, 1H), 6.79-6.71 (m, 1H), 5.50-5.18 (m, 1H), 4.80-4.70 (m, 1H), 4.50-4.20 (m, 1H), 4.05-3.90 (m, 1H), 3.85-3.70 (m, 1H), 3.30-2.83 (m, 2H), 2.80-2.70 (m, 1H), 2.35-2.20 (m, 1H), 2.00-1.80 (m, 2H), 1.70-1.50 (m, 6H), 1.27 (s, 3H), 0.82-0.66 (m, 4H). m/z: ES⁺ [M+H]⁺=519.20.

### Example 3.06: ((1S,5R)-6-(6-(4-fluoro-2-(1-isopropyl-1H-imidazol-5-yl)phenoxy)-1,2,4-triazin-5-yl)-2,6-diazabicyclo[3.2.1]octan-2-yl)((S)-2-methylpyrrolidin-2-yl)methanone

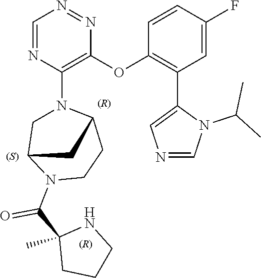

Colourless solid (73 mg, 32% over 2 steps).

¹H NMR (400 MHz, DMSO-d₆, 300 K) δ 8.55-8.40 (m, 1H), 7.95-7.70 (m, 1H), 7.62-7.49 (m, 1H), 7.48-7.20 (m, 2H), 6.80-6.60 (m, 1H), 5.71-5.03 (m, 1H), 4.93-4.61 (m, 1H), 4.59-4.03 (m, 2H), 4.02-3.69 (m, 1H), 3.68-3.49 (m, 1H), 3.18-2.92 (m, 1H), 2.91-2.77 (m, 1H), 2.72-2.58 (m, 1H), 2.35-2.18 (m, 1H), 2.15-1.80 (m, 2H), 1.77-1.47 (m, 5H), 1.45-1.35 (m, 1H), 1.35-1.10 (m, 9H). m/z: ES⁺ [M+H]⁺=521.25.

### Example 3.07: ((1S,5R)-6-(6-(4-fluoro-2-(2-isopropyl-1H-imidazol-1-yl)phenoxy)-1,2,4-triazin-5-yl) 2,6-diazabicyclo[3.2.1]octan-2-yl)((S)-2-methylpyrrolidin-2-yl)methanone

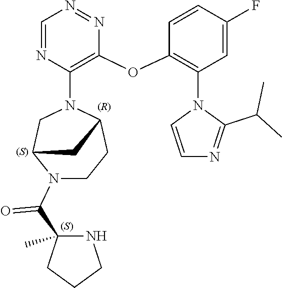

Colourless solid (15 mg, 14% over 2 steps).

¹H NMR (400 MHz, DMSO-d₆, 300 K) δ 8.55-8.40 (m, 1H), 7.72-7.60 (m, 2H), 7.59-7.45 (m, 1H), 7.05-6.95 (m, 1H), 6.85-6.70 (m, 1H), 5.70-5.00 (m, 1H), 4.75-4.65 (m, 1H), 4.60-4.00 (m, 1H), 3.98-3.68 (m, 1H), 3.60-3.35 (m, 1H), 3.20-2.55 (m, 4H), 2.34-2.10 (m, 1H), 2.04-1.80 (m, 2H), 1.80-1.40 (m, 6H), 1.28 (s, 3H), 1.20-0.88 (m, 6H). m/z: ES⁺ [M+H]⁺=521.27.

### Example 3.08: (1S,5R)-2-(L-prolyl)-6-(6-(2-(2-cyclopropyl-5-methyl-1H-imidazol-1-yl)-4-fluorophenoxy)-1,2,4-triazin-5-yl)-2,6-diazabicyclo[3.2.1]octane

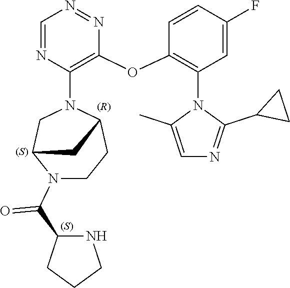

Colourless solid (43 mg, 34% over 2 steps).

¹H NMR (400 MHz, DMSO-d₆, 300 K) δ 8.64-8.32 (m, 1H), 7.78-7.60 (m, 2H), 7.59-7.46 (m, 1H), 6.61-6.19 (m, 1H), 5.25-4.34 (m, 2H), 4.28-3.69 (m, 3H), 3.68-3.46 (m, 1H), 3.19-2.84 (m, 2H), 2.76-2.53 (m, 2H), 2.09-1.79 (m, 6H), 1.79-1.50 (m, 5H), 1.48-1.29 (m, 1H), 0.82-0.28 (m, 4H). m/z: ES⁺ [M+H]⁺=519.25.

### Example 3.09: ((1S,5R)-6-(6-(2-(4-cyclopropylpyrimidin-5-yl)-4-fluorophenoxy)-1,2,4-triazin-5-yl)-2,6-diazabicyclo[3.2.1]octan-2-yl) ((S)-2-methylpyrrolidin-2-yl) methanone

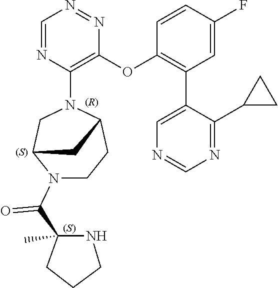

Colourless solid (74 mg, % over 3 steps).

¹H NMR (400 MHz, DMSO-d₆, 300 K) δ 8.93-8.86 (m, 1H), 8.48-8.40 (m, 2H), 7.58 (s, 1H), 7.48-7.43 (m, 2H), 5.33-5.08 (m, 1H), 4.83-4.68 (m, 1H), 4.45-4.19 (m, 1H), 3.89-3.86 (m, 1H), 3.72-3.53 (m, 1H), 3.53-3.50 (m, 1H), 3.32-2.83 (m, 2H), 2.67-2.61 (m, 1H), 2.37-2.07 (m, 1H), 1.90-1.30 (m, 8H), 1.26 (s, 3H), 1.10-0.85 (m, 3H), 0.75-0.56 (m, 1H). m/z: ES⁺ [M+H]⁺=531.25.

### Example 3.10: N-ethyl-N-(5-fluoro-2-((5-((1S,5R)-2-((S)-2-ethylpyrrolidine-2-carbonyl)-2,6-diazabicyclo[3.2.1]octan-6-yl)-1,2,4-triazin-6-yl)oxy)phenyl)isobutyramide

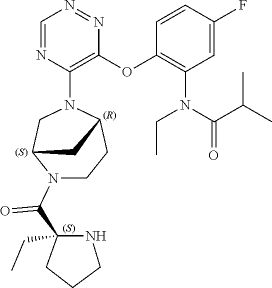

Colourless solid (31 mg, 22% over 2 steps).

¹H NMR (400 MHz, CDCl₃, 300 K) δ 8.47 (s, 1H), 7.45-7.33 (m, 1H), 7.26-7.15 (m, 1H), 7.09-7.00 (m, 1H), 5.85-5.50 (m, 4H), 5.10-5.00 (m, 1H), 4.20-3.80 (m, 4H), 3.50-3.20 (m, 2H), 3.11-2.91 (m, 2H), 2.60-2.35 (m, 1H), 2.30-2.00 (m, 4H), 2.00-1.70 (m, 4H), 1.10-0.98 (m, 9H), 0.90-0.75 (m, 2H), 0.67-0.62 (m, 1H). m/z: ES⁺ [M+H]⁺=540.50.

### Example 3.11: ((1S,3S,5S)-2-azabicyclo[3.1.0]hexan-3-yl)((1S,5R)-6-(6-(2-(4-cyclopropylpyrimidin-5-yl)-4-fluorophenoxy)-1,2,4-triazin-5-yl)-2,6-diazabicyclo[3.2.1]octan-2-yl)methanone

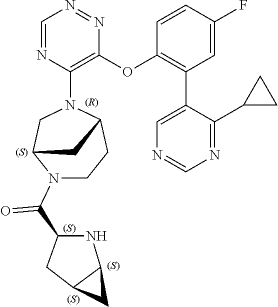

Colourless solid (8 mg, 5% over 2 steps).

¹H NMR (400 MHz, DMSO-d₆, 300 K) δ 8.80-8.98 (m, 1H), 8.85-8.48 (m, 2H), 7.68 (s, 1H), 7.40-751 (m, 2H), 5.52-4.68 (m, 2H), 4.35-3.80 (m, 2H), 3.50-3.44 (m, 2H), 3.03-2.70 (m, 1H), 2.96-2.62 (m, 1H), 2.71-2.28 (m, 2H), 2.00-1.13 (m, 7H), 1.10-0.82 (m, 3H), 0.78-0.55 (m, 2H), 0.28-0.13 (m, 1H). m/z: ES⁺ [M+H]⁺=529.40.

### Example 3.12: ((1S,5R)-6-(6-(2-(4-cyclopropylpyridin-3-yl)-4-fluorophenoxy)-1,2,4-triazin-5-yl)-2,6-diazabicyclo[3.2.1]octan-2-yl)((S)-2-ethylpyrrolidin-2-yl)methanone

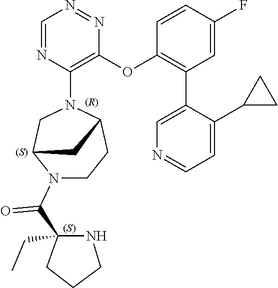

Colourless solid (66 mg, 34% over 2 steps).

¹H NMR (400 MHz, DMSO-d₆, 300 K) δ 8.50-8.25 (m, 2H), 8.16 (s, 1H), 7.55-7.53 (m, 1H), 7.46-7.33 (m, 2H), 6.80 (s, 1H), 5.21 (s, 1H), 4.67 (s, 1H), 4.29-4.04 (m, 1H), 3.78-3.73 (m, 1H), 3.46-3.44 (m, 1H), 2.80 (s, 2H), 2.61 (s, 1H), 2.10-1.90 (m, 1H), 1.83-1.75 (m, 2H), 1.73-1.48 (m, 7H), 1.44-1.19 (m, 1H), 0.93 (s, 1H), 0.83-0.50 (m, 6H). m/z: ES⁺ [M+H]⁺=544.25.

### Example 3.13: ((1S,3S,5S)-2-azabicyclo[3.1.0]hexan-3-yl)((1S,5R)-6-(6-(2-(4-cy clopropylpyridin-3-yl)-4-fluorophenoxy)-1,2,4-triazin-5-yl)-2,6-diazabicyclo[3.2.1]octan-2-yl)methanone

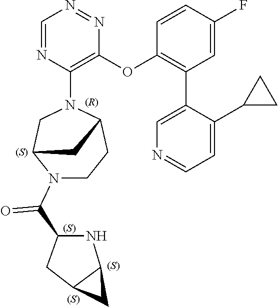

Colourless solid (45 mg, 40% over 2 steps).

¹H NMR (400 MHz, DMSO-d₆, 300 K) δ 8.44-8.41 (m, 1H), 8.35-8.13 (m, 1H), 8.16 (s, 1H), 7.54-7.52 (m, 1H), 7.45-7.41 (m, 1H), 7.40-7.33 (m, 1H), 6.92-6.79 (m, 1H), 5.13-4.47 (m, 2H), 4.37-4.10 (m, 1H), 4.05-3.77 (m, 1H), 3.74-3.37 (m, 2H), 2.62 (s, 2H), 2.38-2.32 (m, 1H), 1.94-1.63 (m, 3H), 1.62-1.17 (m, 4H), 0.89-0.85 (m, 1H), 0.59-0.50 (m, 4H), 0.21 (s, 1H). m/z: ES⁺ [M+H]⁺=528.20.

### Example 3.14: 6-(5-Fluoro-2-((5-((1S,5R)-2-((S)-2-methylpyrrolidine-2-carbonyl)-2,6-diazabicyclo[3.2.1]octan-6-yl)-1,2,4-triazin-6-yl)oxy)phenyl)-1-methylpyridin-2(1H)-one

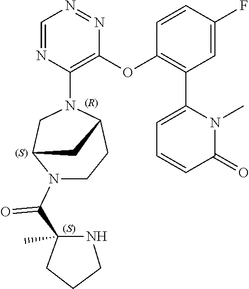

Colourless solid (41 mg, 26% over 2 steps).

¹H NMR (400 MHz, DMSO-d₆, 300 K) δ 8.44-8.39 (m, 1H), 7.76-7.64 (m, 1H), 7.47-7.19 (m, 4H), 6.26-6.12 (m, 1H), 5.60-5.20 (m, 1H), 5.08-4.68 (m, 1H), 4.57-3.95 (m, 2H), 3.94-3.68 (m, 1H), 3.66-3.49 (m, 1H), 343-3.36 (m, 3H), 3.20-2.93 (m, 1H), 2.89-2.75 (m, 1H), 2.66-2.60 (m, 1H), 1.98-1.82 (m, 2H), 1.85-1.55 (m, 6H), 1.38-1.23 (m, 3H). m/z: ES⁺ [M+H]⁺=520.35.

### Example 3.15: (7-azabicyclo[2.2.1]heptan-1-yl)((1S,5R)-6-(6-(2-(2-cyclopropyl-5-methyl-1H-imidazol-1-yl)-4-fluorophenoxy)-1,2,4-triazin-5-yl)-2,6-diazabicyclo[3.2.1]octan-2-yl)methanone

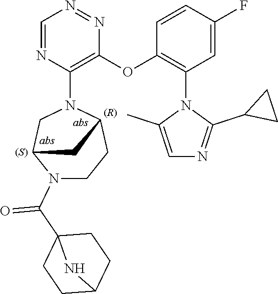

Colourless solid (18 mg, 13% over 2 steps).

¹H NMR (400 MHz, DMSO-d₆, 300 K) δ 8.49-8.46 (m, 1H), 7.72-7.70 (m, 1H), 7.69-7.62 (m, 1H), 7.56-7.52 (m, 1H), 6.54-6.43 (m, 1H), 6.14-6.08 (m, 1H), 5.25-4.77 (m, 1H), 4.72-4.43 (m, 1H), 4.21-3.85 (m, 1H), 3.76-3.54 (m, 3H), 3.15-2.55 (m, 2H), 2.07-1.83 (m, 4H), 1.75-1.56 (m, 8H), 1.36 (s, 3H), 0.73-0.44 (m, 4H). m/z: ES⁺ [M+H]⁺=545.25.

### Example 3.16: ((1S,5R)-6-(6-(2-(2-cyclopropylpyridin-3-yl)-4-fluorophenoxy)-1,2,4-triazin-5-yl)-2,6-diazabicyclo[3.2.1]octan-2-yl)((S)-2-ethylpyrrolidin-2-yl)methanone

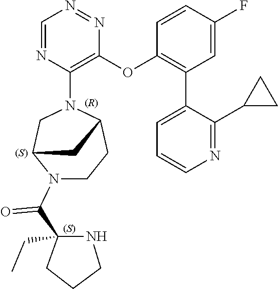

Colourless solid (19 mg, 18% over 2 steps).

¹H NMR (400 MHz, DMSO-d₆, 300 K) δ 8.64-8.12 (m, 2H), 7.62-7.50 (m, 1H), 7.48-7.23 (m, 3H), 7.18-6.86 (m, 1H), 5.42-4.93 (m, 1H), 4.86-4.60 (m, 1H), 4.62-4.18 (m, 1H), 4.16-4.00 (m, 1H), 4.00-3.60 (m, 1H), 3.60-3.42 (m, 1H), 2.90-2.76 (m, 1H), 2.62-2.56 (m, 1H), 2.30-2.08 (m, 1H), 1.98-1.72 (m, 2H), 1.70-1.42 (m, 8H), 1.60-1.08 (m, 1H), 0.96-0.60 (m, 6H), 0.64-0.20 (m, 1H). m/z: ES⁺ [M+H]⁺=544.45.

### Example 3.17: ((1S,5R)-6-(6-(2-(5-cyclopropylpyrimidin-4-yl)-4-fluorophenoxy)-1,2,4-triazin-5-yl)-2,6-diazabicyclo[3.2.1]octan-2-yl)((S)-2-ethylpyrrolidin-2-yl)methanone

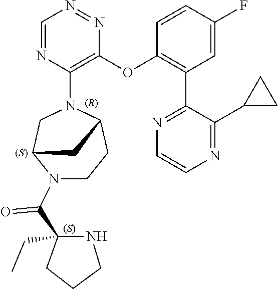

Off-white solid (6 mg, 6% over 2 steps).

¹H NMR (400 MHz, DMSO-d₆, 300 K) δ 9.00-8.91 (m, 1H), 8.45-8.41 (m, 2H), 7.59-7.55 (m, 1H), 7.49-7.44 (m, 2H), 5.23-5.00 (m, 1H), 4.75-4.69 (m, 1H), 4.23-4.16 (m, 1H), 3.96-3.74 (m, 1H), 3.48-3.44 (m, 1H), 2.80 (s, 2H), 2.60 (s, 1H), 2.33-2.12 (m, 1H), 1.86-1.75 (m, 2H), 1.62-1.61 (m, 8H), 1.44-1.43 (m, 1H), 0.87-0.75 (m, 7H). m/z: ES⁺ [M+H]⁺=545.25.

### Example 3.18: (1S,5R)-6-(6-[2-(5-cyclopropyl-2-isopropylimidazol-1-yl)-4-fluorophenoxy]-1,2,4-triazin-5-yl)-2-(hexahydropyrrolizine-7a-carbonyl)-2,6-diazabicyclo[3.2.1]octane

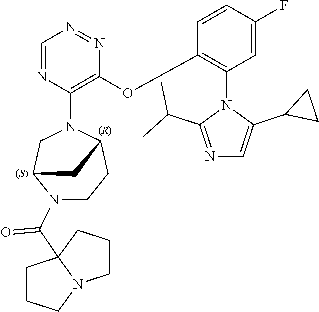

Colourless solid (31 mg, 29%).

1H NMR (400 MHz, DMSO-d₆, 300 K) δ 8.56-8.44 (m, 1H), 7.84-7.71 (m, 2H), 7.63-7.49 (m, 1H), 6.61-6.13 (m, 1H), 5.48-4.83 (m, 1H), 4.83-4.41 (m, 1H), 4.41-4.08 (m, 1H), 3.92-3.49 (m, 2H), 3.18-2.79 (m, 3H), 2.79-2.56 (m, 3H), 2.36-2.01 (m, 2H), 2.01-1.72 (m, 6H), 1.72-1.42 (m, 4H), 1.42-1.23 (m, 1H), 1.10-1.01 (m, 3H), 1.01-0.79 (m, 3H), 1.74-0.28 (m, 4H). m/z: ES⁺ [M+H]⁺=587.20.

### Example 3.19: ((1S,5R)-6-(6-(2-(2-(tert-butyl)-5-methyl-1H-imidazol-1-yl)-4-fluorophenoxy)-1,2,4-triazin-5-yl)-2,6-diazabicyclo[3.2.1]octan-2-yl)((S)-2-methylpyrrolidin-2-yl)methanone

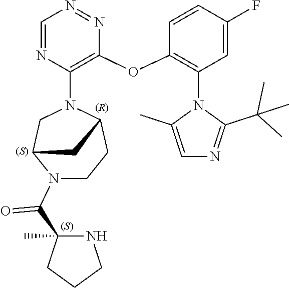

Colourless solid, formic acid salt (56 mg, 59% over 2 steps).

¹H NMR (400 MHz, DMSO-d₆, 300 K) δ 8.46-8.34 (m, 1H), 8.24 (s, 1H), 7.89-7.32 (m, 3H), 6.84-6.29 (m, 1H), 5.24-5.03 (m, 1H), 4.86-4.58 (m, 1H), 4.58-4.21 (m, 1H), 4.03-3.57 (m, 2H), 3.28-2.11 (m, 3H), 2.43-1.81 (m, 7H), 1.81-1.42 (m, 8H), 1.19-0.89 (m, 9H). m/z: ES⁺ [M+H]⁺=549.30.

### Example 3.20: ((1S,5R)-6-(6-(2-(3-cyclopropylpyrazin-2-yl)-4-fluorophenoxy)-1,2,4-triazin-5-yl)-2,6-diazabicyclo[3.2.1]octan-2-yl)((S)-2-ethylpyrrolidin-2-yl)methanone

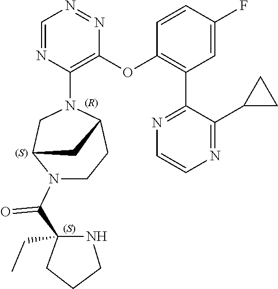

Colourless solid, bis formic acid salt (5 mg, 3% over 2 steps).

¹H NMR (400 MHz, DMSO-d₆, 300 K) δ 8.60-8.30 (m, 3H), 8.29-8.18 (m, 2H), 7.67-7.52 (m, 1H), 7.51-7.40 (m, 2H), 5.25-5.01 (m, 1H), 4.74-4.62 (m, 1H), 4.18-4.08 (m, 3H), 3.54-3.32 (m, 2H), 3.18-2.88 (m, 2H), 2.80-2.64 (m, 1H), 2.31-2.05 (m, 1H), 2.02-1.50 (m, 9H), 1.44-1.02 (m, 1H), 0.98-0.41 (m, 7H). m/z: ES⁺ [M+H]⁺=545.25.

### Example 3.21: ((1S,5R)-6-(6-(2-(5-cyclopropyl-2-methyl-1H-imidazol-1-yl)-4-fluorophenoxy)-1,2,4-triazin-5-yl)-2,6-diazabicyclo[3.2.1]octan-2-yl)((S)-2-methylpyrrolidin-2-yl)methanone

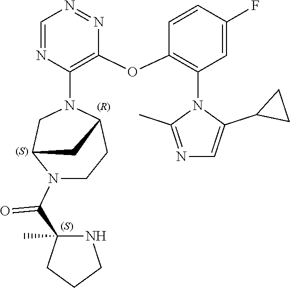

Colourless solid, trifluoroacetic acid salt (24 mg, 8% over 2 steps).

¹H NMR (400 MHz, DMSO-d₆, 300 K) δ 9.46-9.35 (m, 1H), 8.91-8.84 (m, 2H), 8.71-8.57 (m, 1H), 7.97-7.68 (m, 3H), 7.44-7.36 (m, 1H), 5.30-4.71 (m, 2H), 4.35-4.00 (m, 1H), 3.99-3.74 (m, 2H), 3.58-2.80 (m, 3H), 2.45-2.39 (m, 3H), 2.36-2.10 (m, 2H), 2.10-1.89 (m, 4H), 1.88-1.66 (m, 2H), 1.66-1.40 (m, 4H), 0.84-0.71 (m, 3H), 0.67-0.51 (m, 1H). m/z: ES⁺ [M+H]⁺=533.25.

### Example 3.22: 2-((5-((1S,5R)-2-((1S,2S,5R)-3-azabicyclo[3.1.0]hexane-2-carbonyl)-2,6-diazabicyclo[3.2.1]octan-6-yl)-1,2,4-triazin-6-yl)oxy)-5-fluoro-N-isopropyl-N-methylbenzamide

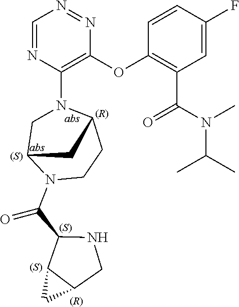

Colourless solid, formic acid salt (43 mg, 35% over 2 steps).

¹H NMR (400 MHz, DMSO-d₆, 300 K) δ 8.58-8.38 (m, 1H), 8.23-8.21 (m, 1H), 7.59-7.21 (m, 3H), 5.26-4.77 (m, 2H), 4.53-3.97 (m, 3H), 3.84-3.26 (m, 2H), 3.09-2.80 (m, 2H), 2.67-2.62 (m, 3H), 2.18-1.63 (m, 4H), 1.52-1.47 (m, 3H), 0.94-0.53 (m, 7H), 0.45-0.35 (m, 2H). m/z: ES⁺ [M+H]⁺=510.25.

### Example 3.23 and 3.24: (1S,5R)-6-(6-(2-(2-cyclopropyl-5-methyl-1H-imidazol-1-yl)-4-fluorophenoxy)-1,2,4-triazin-5-yl)-2,6-diazabicyclo[3.2.1]octan-2-yl)((3aS,6aS)-hexahydrocyclopenta[c]pyrrol-3a(1H)-yl)methanone and (1S,5R)-6-(6-(2-(2-cyclopropyl-5-methyl-1H-imidazol-1-yl)-4-fluorophenoxy)-1,2,4-triazin-5-yl)-2,6-diazabicyclo[3.2.1]octan-2-yl)((3aR,6aR)-hexahydrocyclopenta[c]pyrrol-3a(1H)-yl)methanone

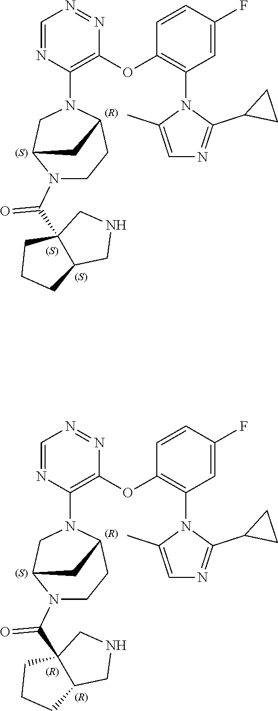

Separation by chiral HPLC gave two single stereoisomers:

Peak #1: light yellow solid (9 mg, 2% over 2 steps).

¹H NMR (400 MHz, DMSO-d₆, 300 K) δ 8.64-8.31 (m, 1H), 7.82-7.42 (m, 3H), 7.63-7.36 (m, 1H), 5.39-4.46 (m, 2H), 4.23-3.56 (m, 2H), 3.21-3.19 (m, 1H), 3.14-2.75 (m, 4H), 2.47-2.36 (m, 1H), 2.01-1.79 (m, 7H), 1.79-1.56 (m, 5H), 1.56-1.42 (m, 2H), 1.42-1.25 (m, 2H), 0.90-0.31 (m, 4H). m/z: ES⁺ [M+H]⁺=559.10.

Peak #2: colourless solid (10 mg, 2% over 2 steps).

¹H NMR (400 MHz, DMSO-d₆, 300 K) δ 8.64-8.31 (m, 1H), 7.82-7.42 (m, 3H), 7.63-7.36 (m, 1H), 5.39-4.36 (m, 3H), 4.31-3.91 (m, 1H), 3.90-3.73 (m, 1H), 3.61-3.59 (m, 1H), 3.14-2.75 (m, 3H), 2.01-1.79 (m, 8H), 1.79-1.56 (m, 4H), 1.56-1.27 (m, 4H), 1.27-1.21 (m, 1H), 0.81-0.67 (m, 2H), 0.63-0.32 (m, 2H). m/z: ES⁺ [M+H]⁺=559.25.

### Example 3.25 and 3.26: 5-fluoro-N-isopropyl-N-methyl-2-((5-((1S,5R)-2-((3aR,6aR)-octahydrocyclopenta[c]pyrrole-3a-carbonyl)-2,6-diazabicyclo[3.2.1]octan-6-yl)-1,2,4-triazin-6-yl)oxy)benzamide and 5-fluoro-N-isopropyl-N-methyl-2-((5-((1S,5R)-2-((3aS,6aS)-octahydrocyclopenta[c]pyrrole-3a-carbonyl)-2,6-diazabicyclo[3.2.1]octan-6-yl)-1,2,4-triazin-6-yl)oxy)benzamide

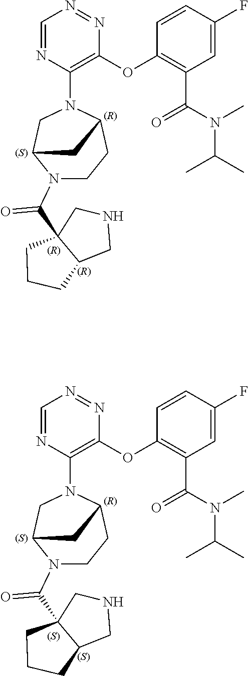

Separation by chiral HPLC gave two single stereoisomers:

Peak #1: Colourless solid (29 mg, 3% over 2 steps).

¹H NMR (400 MHz, DMSO-d₆, 300 K) δ 8.57-8.45 (m, 1H), 7.68-7.16 (m, 3H), 5.28-4.96 (m, 1H), 4.85-4.57 (m, 1H), 4.56-3.78 (m, 3H), 3.78-3.52 (m, 2H), 3.30-3.20 (m, 1H), 3.04-2.86 (m, 2H), 2.86-2.75 (m, 1H), 2.75-2.65 (m, 2H), 2.59-2.52 (m, 2H), 2.47-2.38 (m, 1H), 2.13-1.55 (m, 8H), 1.53-1.21 (m, 2H), 1.19-0.60 (m, 6H). m/z: ES⁺ [M+H]⁺=538.30.

Peak #2: Colourless solid (31 mg, 3% over 2 steps).

¹H NMR (400 MHz, DMSO-d₆, 300 K) δ 8.58-8.44 (m, 1H), 7.57-7.23 (m, 3H), 5.31-4.93 (m, 1H), 4.86-4.57 (m, 1H), 4.54-3.78 (m, 3H), 3.77-3.49 (m, 3H), 3.12-2.86 (m, 2H), 2.85-2.67 (m, 3H), 2.58-2.52 (m, 2H), 2.48-2.40 (m, 1H), 2.10-1.57 (m, 8H), 1.55-1.26 (m, 2H), 1.18-0.59 (m, 6H). m/z: ES⁺ [M+H]⁺=538.25.

### Example 3.27 and 3.28: N-ethyl-N-(5-fluoro-2-((5-((1S,5R)-2-((3aS,6aS)-octahydrocyclopenta[c]pyrrole-3a-carbonyl)-2,6-diazabicyclo[3.2.1]octan-6-yl)-1,2,4-triazin-6-yl)oxy)phenyl)isobutyramide and N-ethyl-N-(5-fluoro-2-((5-((1S,5R)-2-((3aR,6aR)-octahydrocyclopenta[c]pyrrole-3a-carbonyl)-2,6-diazabicyclo[3.2.1]octan-6-yl)-1,2,4-triazin-6-yl)oxy)phenyl)isobutyramide

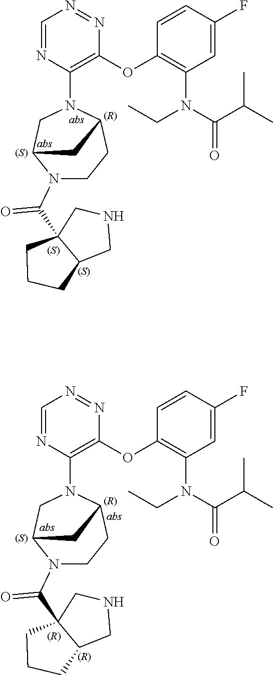

Separation by chiral HPLC gave two single stereoisomers:

Peak #1: off-white solid (61 mg, 8% over 2 steps).

¹H NMR (400 MHz, DMSO-d₆, 300 K) δ 8.70-8.54 (m, 1H), 7.88-7.10 (m, 3H), 5.18-4.65 (m, 2H), 4.35-4.12 (m, 1H), 3.94-3.85 (m, 2H), 3.72-3.53 (m, 2H), 3.11-2.81 (m, 4H), 2.52-2.44 (m, 2H), 2.02-1.64 (m, 9H), 1.43-1.15 (m, 2H), 1.04-0.88 (m, 6H), 0.76-0.65 (m, 3H). m/z: ES⁺ [M+H]⁺=552.30.

Peak #2: off-white solid (36 mg, 5% over 2 steps).

¹H NMR (400 MHz, DMSO-d₆, 300 K) δ 8.70-8.54 (m, 1H), 7.88-7.10 (m, 3H), 5.18-4.65 (m, 2H), 4.35-4.12 (m, 1H), 3.94-3.85 (m, 2H), 3.72-3.53 (m, 2H), 3.11-2.81 (m, 4H), 2.52-2.44 (m, 2H), 2.02-1.64 (m, 9H), 1.43-1.15 (m, 2H), 1.04-0.88 (m, 6H), 0.76-0.65 (m, 3H). m/z: ES⁺ [M+H]⁺=552.30.

### Example 3.29: N-(2-((5-((1S,5R)-2-((1S,2S,5R)-3-azabicyclo[3.1.0]hexane-2-carbonyl)-2,6-diazabicyclo[3.2.1]octan-6-yl)-1,2,4-triazin-6-yl)oxy)-5-fluorophenyl)-N-ethylisobutyramide

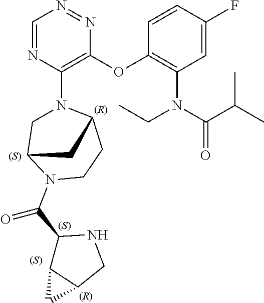

Off-white solid (26 mg, 18% over 2 steps).

¹H NMR (400 MHz, DMSO-d₆, 300 K) δ 8.59-8.49 (m, 1H), 8.20-8.05 (m, 1H), 7.58-7.35 (m, 3H), 5.18-4.95 (m, 2H), 4.02-3.99 (m, 2H), 3.98-3.90 (m, 2H), 3.66-3.55 (m, 1H), 3.44-3.32 (m, 1H), 3.16-3.06 (m, 1H), 2.91-2.60 (m, 2H), 2.42-2.30 (m, 1H), 2.20-1.95 (m, 4H), 1.93-1.51 (m, 2H), 0.98-0.91 (m, 6H), 0.79-0.68 (m, 3H), 0.47-0.31 (m, 2H). m/z: ES⁺ [M+H]⁺=524.15.

### Example 3.30 and 3.31: (2R)-1-((1S,5R)-6-(6-(2-(2,5-dicyclopropyl-1H-imidazol-1-yl)-4-fluorophenoxy)-1,2,4-triazin-5-yl)-2,6-diazabicyclo[3.2.1]octan-2-yl)-2-((R)-pyrrolidin-3-yl)propan-1-one and (2R)-1-((1S,5R)-6-(6-(2-(2,5-dicyclopropyl-1H-imidazol-1-yl)-4-fluorophenoxy)-1,2,4-triazin-5-yl)-2,6-diazabicyclo[3.2.1]octan-2-yl)-2-((S)-pyrrolidin-3-yl)propan-1-one

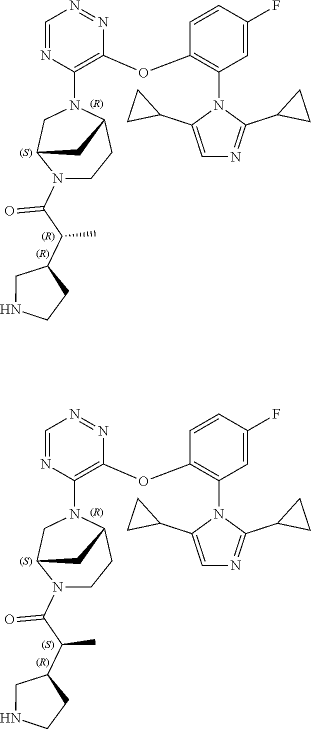

Separation by chiral HPLC gave two single stereoisomers:

Peak #1: Brown semi-solid (3 mg, 4% over 2 steps).

¹H NMR (400 MHz, DMSO-d₆, 300 K) δ 8.62-8.41 (m, 1H), 7.81-7.73 (m, 2H), 7.69-7.62 (m, 1H), 6.51-6.31 (m, 1H), 5.31-4.41 (m, 2H), 4.35-3.71 (m, 2H), 3.65-3.55 (m, 1H), 3.54-3.50 (m, 1H), 3.25-3.03 (m, 2H), 3.00-2.93 (m, 1H), 2.90-2.70 (m, 1H), 2.69-2.60 (m, 1H), 2.35-2.11 (m, 1H), 2.05-1.85 (m, 3H), 1.84-1.53 (m, 2H), 1.50-1.05 (m, 4H), 1.02-0.95 (m, 3H), 0.93-0.65 (m, 4H), 0.63-0.20 (m, 4H). m/z: ES⁺ [M+H]⁺=573.50.

Peak #2: Brown semi-solid (3 mg, 5% over 2 steps).

¹H NMR (400 MHz, DMSO-d₆, 300 K) δ 8.63-8.42 (m, 1H), 7.85-7.71 (m, 2H), 7.68-7.61 (m, 1H), 6.52-6.30 (m, 1H), 5.31-4.61 (m, 2H), 4.35-3.51 (m, 4H), 3.25-3.13 (m, 1H), 3.00-2.93 (m, 1H), 2.90-2.70 (m, 2H), 2.69-2.60 (m, 1H), 2.35-2.11 (m, 1H), 2.05-1.85 (m, 3H), 1.84-1.54 (m, 3H), 1.49-1.25 (m, 3H), 1.02-0.93 (m, 3H), 0.92-0.63 (m, 4H), 0.62-0.21 (m, 4H). m/z: ES⁺ [M+H]⁺=573.45.

### Example 3.32: ((1S,5R)-6-(6-(2-(2-cyclopropyl-5-methyl-1H-imidazol-1-yl)-4-fluorophenoxy)-1,2,4-triazin-5-yl)-2,6-diazabicyclo[3.2.1]octan-2-yl)(tetrahydro-1H-pyrrolizin-7a(5H)-yl)methanone

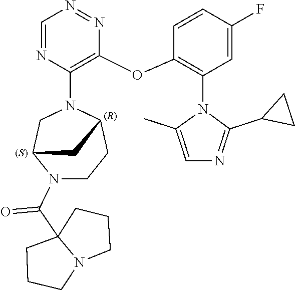

Colourless solid (20 mg, 25%).

¹H NMR (400 MHz, DMSO-d₆, 300 K) δ 8.58-8.24 (m, 1H), 7.79-7.60 (m, 2H), 7.59-7.48 (m, 1H), 6.66-6.36 (m, 1H), 5.60-4.71 (m, 1H), 4.71-4.15 (m, 1H), 3.99-3.70 (m, 1H), 3.70-3.50 (m, 1H), 3.50-3.39 (m, 1H), 3.00-2.85 (m, 2H), 2.67-2.59 (m, 2H), 2.30-2.09 (m, 2H), 2.00-1.70 (m, 10H), 1.70-1.52 (m, 4H), 1.50-1.28 (m, 1H), 0.80-0.38 (m, 4H). m/z: ES⁺ [M+H]⁺=559.25.

### Example 3.33: (7-azabicyclo[2.2.1]heptan-1-yl)((1S,5R)-6-(6-(2-(2,5-dicyclopropyl-1H-imidazol-1-yl)-4-fluorophenoxy)-1,2,4-triazin-5-yl)-2,6-diazabicyclo[3.2.1]octan-2-yl)methanone

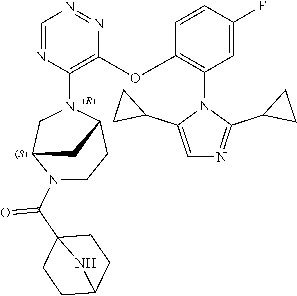

Colourless oil (21 mg, 33% over 2 steps).

¹H NMR (400 MHz, CDCl₃, 300 K) δ 8.75-8.20 (m, 1H), 7.75-7.35 (m, 1H), 7.35-7.29 (m 1H), 7.29-7.10 (m, 2H), 6.60-6.40 (m, 1H), 5.00-4.60 (m, 1H), 4.60-4.12 (m, 1H), 4.10-3.10 (m, 3H), 3.20-2.70 (m, 1H), 2.30-2.00 (m, 5H), 2.00-1.85 (m, 4H), 1.80-1.70 (m, 2H), 1.55-1.50 (m, 1H), 1.50-1.40 (m, 2H), 1.35-1.20 (m, 1H), 1.00-0.65 (m, 5H), 0.62-0.35 (m, 3H). m/z: ES⁺ [M+H]⁺=571.25.

### Example 3.34 and Example 3.35: ((1S,5R)-6-(6-(2-(2,5-dicyclopropyl-1H-imidazol-1-yl)-4-fluorophenoxy)-1,2,4-triazin-5-yl)-2,6-diazabicyclo[3.2.1]octan-2-yl)((3aR,6aR)-hexahydrocyclopenta[c]pyrrol-3a(1H)-yl)methanone and ((1S,5R)-6-(6-(2-(2,5-dicyclopropyl-1H-imidazol-1-yl)-4-fluorophenoxy)-1,2,4-triazin-5-yl)-2,6-diazabicyclo[3.2.1]octan-2-yl)((3aS,6aS)-hexahydrocyclopenta[c]pyrrol-3a(1H)-yl)methanone

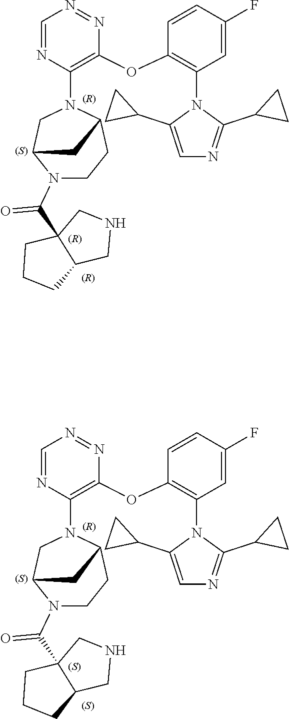

Separation by chiral HPLC gave two single stereoisomers:

Peak #1: Colourless solid (16 mg, 8% over 2 steps). 1H NMR (400 MHz, DMSO-d₆, 300 K) δ 8.58-8.42 (m, 1H), 7.78-7.63 (m, 2H), 7.56-7.40 (m, 1H), 6.41-6.30 (m, 1H), 5.20-5.10 (m, 1H), 4.80-4.74 (m, 2H), 3.90-3.70 (m, 1H), 3.70-3.58 (m, 2H), 3.12-2.70 (m, 3H), 2.46-2.28 (m, 1H), 2.01-1.80 (m, 4H), 1.77-1.50 (m, 4H), 1.50-1.15 (m, 5H), 0.82-0.23 (m, 9H). m/z: ES⁺ [M+H]⁺=585.30.

Peak #2: Colourless solid (18 mg, 9% over 2 steps).

¹H NMR (400 MHz, DMSO-d₆, 300 K) δ 8.58-8.42 (m, 1H), 7.78-7.63 (m, 2H), 7.56-7.40 (m, 1H), 6.41-6.30 (m, 1H), 5.20-5.10 (m, 1H), 4.80-4.74 (m, 2H), 3.90-3.70 (m, 1H), 3.70-3.58 (m, 2H), 3.30-3.22 (m, 1H), 3.12-2.70 (m, 4H), 2.01-1.80 (m, 4H), 1.77-1.50 (m, 4H), 1.50-1.15 (m, 5H), 0.82-0.23 (m, 8H). m/z: ES⁺ [M+H]⁺=585.30.

### Example 3.36: ((1S,5R)-6-(6-(2-(2,5-dicyclopropyl-1H-imidazol-1-yl)-4-fluorophenoxy)-1,2,4-triazin-5-yl)-2,6-diazabicyclo[3.2.1]octan-2-yl)((S)-2-ethylpyrrolidin-2-yl)methanone

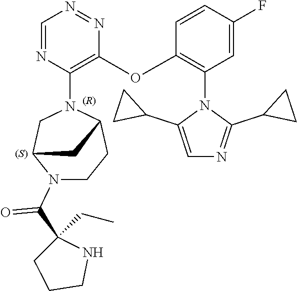

Colourless solid (4 mg, 7% over 2 steps).

¹H NMR (400 MHz, DMSO-d₆, 300 K) δ 8.55-8.45 (m, 1H), 7.80-7.66 (m, 2H), 7.60-7.46 (s, 1H), 6.55-6.35 (m, 1H), 5.30-5.07 (m, 1H), 4.80-4.66 (m, 1H), 4.36-4.05 (m, 1H), 3.95-3.83 (m, 1H), 3.80-3.65 (m, 1H), 3.05-2.75 (m, 2H), 2.67-2.55 (m, 1H), 2.16-2.01 (m, 1H), 1.95-1.79 (m, 2H), 1.75-1.49 (m, 7H), 1.34-1.14 (m, 3H). 0.94-0.64 (m, 7H). 0.54-0.32 (m, 4H). m/z: ES⁺ [M+H]⁺=573.55.

### Example 3.37: 1-((1S,5R)-6-(6-(2-(2,5-dicyclopropyl-1H-imidazol-1-yl)-4-fluorophenoxy)-1,2,4-triazin-5-yl)-2,6-diazabicyclo[3.2.1]octan-2-yl)-2-(difluoromethoxy)ethan-1-one

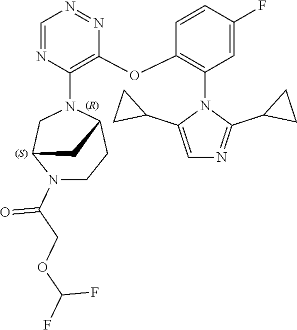

Off-white solid (6 mg, 14%).

¹H NMR (400 MHz, DMSO-d₆, 300 K) 68.70-8.41 (m, 1H), 7.97-7.41 (m, 3H), 7.12-6.51 (m, 1H), 6.50-6.11 (m, 1H), 5.21-4.50 (m, 3H), 4.49-4.05 (m, 1H), 3.95-3.70 (m, 1H), 3.65-3.51 (m, 2H), 3.25-2.85 (m, 1H), 2.11-1.51 (m, 4H), 1.45-1.21 (m, 2H), 0.80-0.20 (m, 8H). m/z: ES⁺ [M+H]⁺=556.15.

### Example 3.38: ((1S,5R)-6-(6-(2-(2-cyclopropyl-5-methyl-1H-imidazol-1-yl)-4-fluorophenoxy)-1,2,4-triazin-5-yl)-2,6-diazabicyclo[3.2.1]octan-2-yl)((S)-2-ethylpyrrolidin-2-yl)methanone

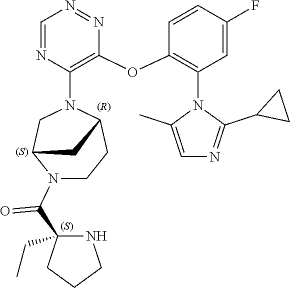

Colourless solid (15 mg, 6% over 2 steps).

¹H NMR (400 MHz, DMSO-d₆, 300 K) δ 8.62-8.40 (m, 1H), 7.83-7.31 (m, 3H), 6.65-6.28 (m, 1H), 5.52-5.11 (m, 1H), 4.95-4.40 (m, 1H), 4.41-4.05 (m, 1H), 4.41-3.66 (m, 1H), 3.65-3.47 (m, 1H), 3.15-2.78 (m, 2H), 2.77-2.57 (m, 2H), 2.19-2.04 (m, 1H), 1.99-1.80 (m, 5H), 1.71-1.55 (m, 7H), 1.47-1.18 (m, 1H), 0.84-0.76 (m, 3H), 0.74-0.62 (m, 2H), 0.61-0.35 (m, 2H). m/z: ES⁺ [M+H]⁺=547.45.

### Example 3.39: ((1S,5R)-6-(6-(2-(2-cyclopropyl-5-isopropyl-1H-imidazol-1-yl)-4-fluorophenoxy)-1,2,4-triazin-5-yl)-2,6-diazabicyclo[3.2.1]octan-2-yl)((3aS,6aS)-hexahydrocyclopenta[c]pyrrol-3a(1H)-yl)methanone and ((1S,5R)-6-(6-(2-(2-cyclopropyl-5-isopropyl-1H-imidazol-1-yl)-4-fluorophenoxy)-1,2,4-triazin-5-yl)-2,6-diazabicyclo[3.2.1]octan-2-yl)((3aR,6aR)-hexahydrocyclopenta[c]pyrrol-3a(1H)-yl)methanone

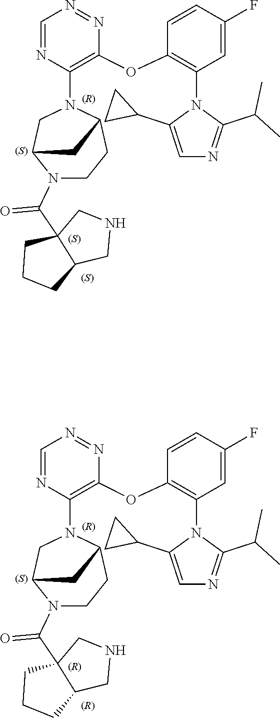

Yellow solid (120 mg, 66% over 2 steps).

m/z: ES⁺ [M+H]⁺=587.32.

### Example 3.40: ((1S,5R)-6-(6-(2-(4-cyclopropyl-1-isopropyl-1H-pyrazol-5-yl)-4-fluorophenoxy)-1,2,4-triazin-5-yl)-2,6-diazabicyclo[3.2.1]octan-2-yl)((3aR,6aR)-hexahydrocyclopenta[c]pyrrol-3a(1H)-yl)methanone

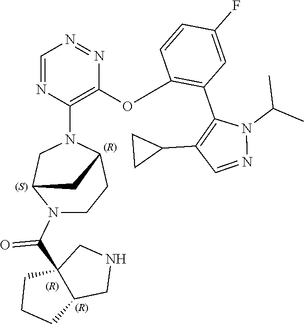

Colourless solid, formic acid salt (16 mg, 17% over 2 steps).

¹H NMR (400 MHz, DMSO-d₆, 300K) δ 8.55-8.43 (m, 1H), 8.26-8.25 (m, 1H), 7.66-7.54 (m, 1H), 7.45-7.41 (m, 2H), 7.15-7.07 (m, 1H), 5.22-4.68 (m, 2H), 4.59-3.98 (m, 3H), 3.95-3.80 (m, 1H), 3.67-3.47 (m, 2H), 3.12-2.99 (m, 4H), 2.97-2.66 (m, 1H), 1.78-1.67 (m, 7H), 1.50-1.46 (m, 1H), 1.40-1.36 (m, 1H), 1.34-1.14 (m, 4H), 1.12-0.90 (m, 3H), 0.64-0.42 (m, 1H), 0.34-0.25 (m, 3H). m/z: ES⁺ [M+H]⁺=587.30.

### Examples 3.41 and 3.42: ((1S,5R)-6-(6-(4-fluoro-2-(2-isopropyl-5-methyl-1H-imidazol-1-yl)phenoxy)-1,2,4-triazin-5-yl)-2,6-diazabicyclo[3.2.1]octan-2-yl)((3aS,6aS)-hexahydrocyclopenta[c]pyrrol-3a(1H)-yl)methanone and ((1S,5R)-6-(6-(4-fluoro-2-(2-isopropyl-5-methyl-1H-imidazol-1-yl)phenoxy)-1,2,4-triazin-5-yl)-2,6-diazabicyclo[3.2.1]octan-2-yl)((3aR,6aR)-hexahydrocyclopenta[c]pyrrol-3a(1H)-yl)methanone

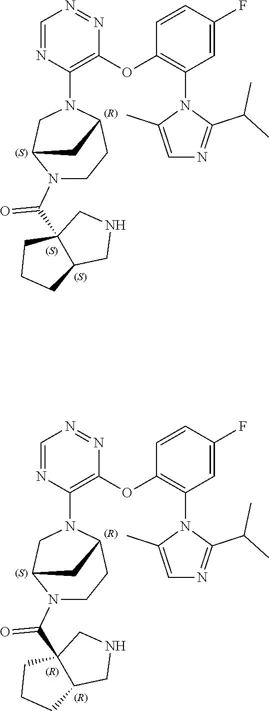

Separation by chiral HPLC gave two single stereoisomers:

Peak #1: light yellow solid (56 mg, 9% over 2 steps).

¹H NMR (400 MHz, DMSO-d₆, 300 K) δ 8.71-7.89 (m, 2H), 7.64-7.53 (m, 3H), 6.85-6.35 (m, 1H), 5.13-4.85 (m, 1H), 4.72-4.14 (m, 1H), 3.95-3.77 (m, 1H), 3.75-3.62 (m, 2H), 3.15-3.08 (m, 3H), 2.96-2.52 (m, 3H), 2.02-1.95 (m, 2H), 1.93-1.81 (m, 5H), 1.80-1.73 (m, 3H), 1.70-1.32 (m, 4H), 1.02-0.85 (m, 6H). m/z: ES⁺ [M+H]⁺=561.20.

Peak #2: colourless solid (56 mg, 9% over 2 steps).

¹H NMR (400 MHz, DMSO-d₆, 300 K) δ 8.71-7.89 (m, 1H), 7.64-7.53 (m, 3H), 6.85-6.35 (m, 1H), 5.13-4.85 (m, 2H), 4.14-3.77 (m, 2H), 3.75-3.62 (m, 2H), 3.15-3.00 (m, 2H), 2.96-2.52 (m, 4H), 2.02-1.70 (m, 8H), 1.70-1.32 (m, 6H), 1.02-0.85 (m, 6H). m/z: ES⁺ [M+H]⁺=561.20.

### Example 3.43: ((1S,5R)-6-(6-(4-fluoro-2-(2-methoxy-5-methyl-1H-imidazol-1-yl)phenoxy)-1,2,4-triazin-5-yl)-2,6-diazabicyclo[3.2.1]octan-2-yl)((S)-2-methylpyrrolidin-2-yl)methanone

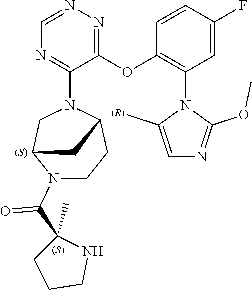

Colourless solid (39 mg, 59%).

¹H NMR (400 MHz, DMSO-d₆, 300 K) δ 8.57-8.38 (m, 1H), 7.70-7.39 (m, 3H), 6.33-6.11 (m, 1H), 5.68-5.10 (m, 1H), 4.94-4.64 (m, 1H), 4.62-4.16 (m, 1H), 4.01-3.52 (m, 5H), 3.25-2.90 (m, 1H), 2.88-2.80 (m, 1H), 2.70-2.58 (m, 1H), 2.40-2.18 (m, 1H), 2.05-1.73 (m, 6H), 1.73-1.48 (m, 5H), 1.42-1.19 (m, 3H). m/z: ES⁺ [M+H]⁺=523.25.

### Example 3.44: ((1S,5R)-6-(6-(2-(4,6-dimethylpyrimidin-5-yl)-4-fluorophenoxy)-1,2,4-triazin-5-yl)-2,6-diazabicyclo[3.2.1]octan-2-yl) ((3aR,6aR)-hexahydrocy clopenta[c]pyrrol-3a(1H)-yl)methanone

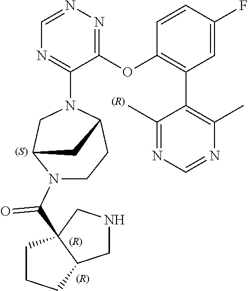

Colourless solid (30 mg, 45%).

¹H NMR (400 MHz, DMSO-d₆, 300 K) δ 8.81-8.77 (m, 1H), 8.45-8.42 (m, 1H), 7.56-7.50 (m, 1H), 7.49-7.37 (m, 2H), 4.88-4.65 (m, 2H), 4.55-4.10 (m, 1H), 3.84-3.65 (m, 3H), 3.49-3.33 (m, 1H), 3.11-3.02 (m, 2H), 2.87-2.83 (m, 1H), 2.33-2.21 (m, 2H), 2.18-2.01 (m, 6H), 1.75-1.43 (m, 8H), 1.37-1.23 (m, 2H). m/z: ES⁺ [M+H]⁺=545.25.

### Example 3.45: ((1S,5R)-6-(6-(2-(5-cyclopropyl-2,4-dimethyl-1H-imidazol-1-yl)-4-fluorophenoxy)-1,2,4-triazin-5-yl)-2,6-diazabicyclo[3.2.1]octan-2-yl)((S)-2-ethylpyrrolidin-2-yl)methanone

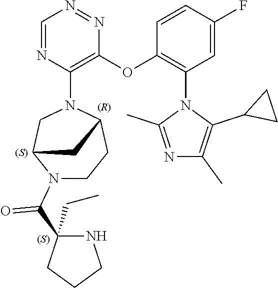

Yellow solid, bis-HCl salt (6 mg, 5% over 2 steps).

¹H NMR (400 MHz, DMSO-d₆, 300 K) δ 15.19-14.50 (m, 1H), 9.55-9.11 (m, 1H), 9.11-8.82 (m, 1H), 8.81-8.40 (m, 1H), 8.01-7.46 (m, 3H), 5.35-4.90 (m, 1H), 4.90-4.70 (m, 1H), 4.32-4.00 (m, 2H), 3.30-3.10 (m, 3H), 3.00-2.90 (m, 2H), 2.41-2.38 (m, 3H), 2.29-2.18 (m, 4H), 2.12-1.85 (m, 7H), 1.85-1.65 (m, 2H), 1.55-1.38 (m, 1H), 0.91-0.80 (m, 4H), 0.71-0.55 (m, 2H), 0.50-0.38 (m, 1H). m/z: ES⁺ [M+H]⁺=561.30.

### Example 3.46: ((1S,5R)-6-(6-(2-(5-cyclopropyl-2-methyl-1H-imidazol-1-yl)-4-fluorophenoxy)-1,2,4-triazin-5-yl)-2,6-diazabicyclo[3.2.1]octan-2-yl)(tetrahydro-1H-pyrrolizin-7a(5H)-yl)methanone

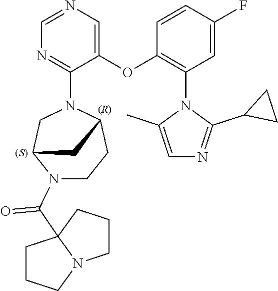

Off-white solid (26 mg, 38%) ¹H NMR (400 MHz, DMSO-d₆, 300 K) δ 8.55-8.46 (m, 1H), 7.68-7.60 (m, 2H), 7.59-7.50 (m, 1H), 6.46-6.36 (m, 1H), 5.65-5.00 (m, 1H), 4.75-4.57 (m, 1H), 4.40-4.24 (m, 1H), 3.96-3.70 (m, 1H), 3.65-3.52 (m, 1H), 3.05-2.92 (m, 2H), 2.72-2.60 (m, 2H), 2.30-2.17 (m, 2H), 2.05-1.90 (m, 4H), 1.90-1.70 (m, 6H), 1.70-1.55 (m, 4H), 1.45-1.30 (m, 1H), 0.62-0.46 (m, 3H), 0.46-0.37 (m, 1H). m/z: ES⁺ [M+H]⁺=559.25.

### Example 3.47: ((1S,5R)-6-(6-(2-(5-cyclopropyl-2-methyl-1H-imidazol-1-yl)-4-fluorophenoxy)-1,2,4-triazin-5-yl)-2,6-diazabicyclo[3.2.1]octan-2-yl)((3aR,6aR)-hexahydrocyclopenta[c]pyrrol-3a(1H)-yl)methanone

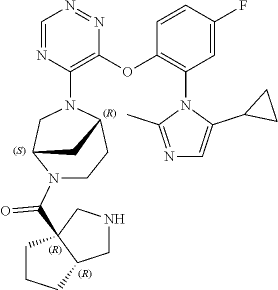

Colourless solid (14 mg, 10% over 2 steps)

¹H NMR (400 MHz, DMSO-d₆, 300 K) δ 8.51-8.47 (m, 1H), 7.67-7.62 (m, 2H), 7.55-7.54 (m, 1H), 6.44-6.34 (m, 1H), 5.14-4.60 (m, 2H), 4.16-3.77 (m, 2H), 3.64-3.57 (m, 1H), 3.04-2.67 (m, 4H), 2.55-2.40 (m, 1H), 2.05-1.75 (m, 7H), 1.72-1.63 (m, 5H), 1.44-1.26 (m, 3H), 0.71-0.43 (m, 3H), 0.43-0.38 (m, 1H). m/z: ES+ [M+H]⁺=559.30.

### Example 3.48: ((1S,5R)-6-(6-(2-(3-cyclopropyl-5-methyl-4H-1,2,4-triazol-4-yl)-4-fluorophenoxy)-1,2,4-triazin-5-yl)-2,6-diazabicyclo[3.2.1]octan-2-yl)((3aR,6aR)-hexahydrocyclopenta[c]pyrrol-3a(1H)-yl)methanone

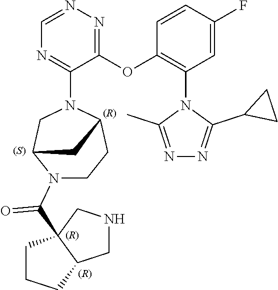

Off-white solid, formic acid salt (35 mg, 14% over 2 steps)

¹H NMR (400 MHz, DMSO-d₆, 300 K) δ 8.64-8.40 (m, 1H), 8.26 (s, 1H), 7.92-7.68 (m, 2H), 7.67-7.51 (m, 1H), 5.26-4.80 (m, 1H), 4.79-4.59 (m, 1H), 4.28-4.11 (m, 1H), 4.01-3.91 (m, 1H), 3.81-3.79 (m, 1H), 3.40-3.31 (m, 3H), 3.16-3.08 (m, 2H), 3.00-2.91 (m, 1H), 2.67-2.59 (m, 1H), 2.15-2.06 (m, 3H), 2.02-1.80 (m, 4H), 1.78-1.60 (m, 4H), 1.56-1.45 (m, 2H), 1.43-1.35 (m, 1H), 0.90-0.70 (m, 4H). m/z: ES⁺ [M+H]⁺=560.25.

### Example 3.49: N-ethyl-N-(5-fluoro-2-((5-((1S,5R)-2-((3aR,6aR)-octahydrocyclopenta[c]pyrrole-3a-carbonyl)-2,6-diazabicyclo[3.2.1]octan-6-yl)-1,2,4-triazin-6-yl)oxy)phenyl)cyclopropane carboxamide

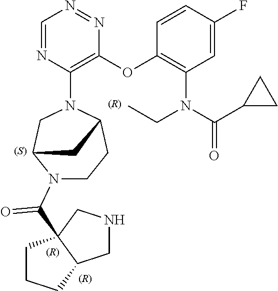

Off-white solid (27 mg, 20% over 2 steps)

¹H NMR (400 MHz, DMSO-d₆, 300 K) δ 8.60-8.40 (m, 1H), 7.71-7.55 (m, 1H), 7.54-7.43 (m, 1H), 7.43-7.35 (m, 1H), 5.31-4.93 (m, 1H), 4.90-4.51 (m, 1H), 4.48-3.89 (m, 2H), 3.89-3.50 (m, 3H), 3.30-3.13 (m, 2H), 3.12-2.87 (m, 2H), 2.85-2.70 (m, 1H), 2.45-2.35 (m, 1H), 2.10-1.58 (m, 9H), 1.50-1.19 (m, 3H), 1.05-0.85 (m, 3H), 0.75-0.50 (m, 3H), 0.50-0.21 (m, 1H). m/z: ES⁺ [M+H]⁺=550.30.

### Example 3.50: ((1S,5R)-6-(6-(2-(3,5-dicyclopropyl-4H-1,2,4-triazol-4-yl)-4-fluorophenoxy)-1,2,4-triazin-5-yl)-2,6-diazabicyclo[3.2.1]octan-2-yl)((3aR,6aR)-hexahydrocy clopenta[c]pyrrol-3a(1H)-yl)methanone

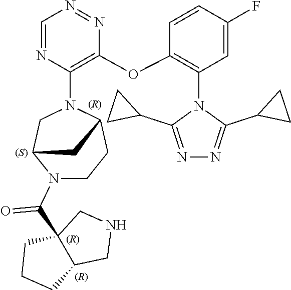

Colourless solid (49 mg, 29% over 2 steps)

¹H NMR (400 MHz, DMSO-d₆, 300 K) δ 8.62-8.32 (m, 1H), 7.91-7.81 (m, 1H), 7.80-7.70 (m, 1H), 7.68-7.49 (m, 1H), 5.40-4.31 (m, 2H), 4.01-3.43 (m, 3H), 3.31-3.00 (m, 3H), 3.00-2.60 (m, 3H), 2.48-2.35 (m, 1H), 2.09-1.38 (m, 11H), 1.38-1.20 (m, 1H), 0.95-0.50 (m, 8H). m/z: ES⁺ [M+H]⁺=586.30.

### Example 3.51: ((1S,5R)-6-(6-(2-(3,5-diisopropyl-4H-1,2,4-triazol-4-yl)-4-fluorophenoxy)-1,2,4-triazin-5-yl)-2,6-diazabicyclo[3.2.1]octan-2-yl)((3aR,6aR)-hexahydrocyclopenta[c]pyrrol-3a(1H)-yl)methanone

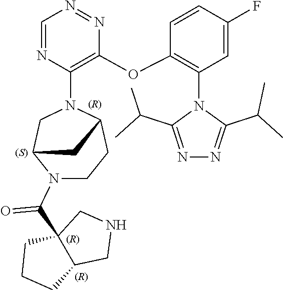

Off-white solid, HCl salt (82 mg, 46% over 2 steps).

¹H NMR (400 MHz, DMSO-d₆, 300 K) δ 9.89-9.49 (m, 1H), 9.49-9.10 (m, 1H), 8.90-8.65 (m, 1H), 8.01-7.95 (m, 1H), 7.95-7.80 (m, 1H), 7.80-7.60 (m, 1H), 5.25-5.15 (m, 1H), 4.95-4.80 (m, 1H), 4.62-4.20 (m, 1H), 4.10-3.83 (m, 1H), 3.83-3.55 (m, 1H), 3.55-3.37 (m, 3H), 3.37-3.09 (m, 2H), 3.09-2.90 (m, 1H), 2.90-2.70 (m, 2H), 2.20-1.66 (m, 8H), 1.60-1.45 (m, 2H), 1.31-0.92 (m, 13H). m/z: ES⁺ [M+H]⁺=590.35.

### Example 3.52: ((1S,5R)-6-(6-(4-fluoro-2-(2-isopropoxy-5-methyl-1H-imidazol-1-yl)phenoxy)-1,2,4-triazin-5-yl)-2,6-diazabicyclo[3.2.1]octan-2-yl)((S)-2-methylpyrrolidin-2-yl)methanone

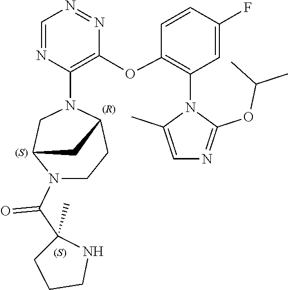

Colourless solid (4 mg, 6% over 2 steps %)

¹H NMR (400 MHz, DMSO-d₆, 300K) δ 8.5-8.43 (m, 1H), 7.68-7.40 (m, 3H), 6.33-6.14 (m, 1H), 5.31-5.16 (m, 1H), 4.87-4.63 (m, 2H), 4.42-4.21 (m, 1H), 3.98-3.72 (m, 1H), 3.69-3.61 (m, 1H), 3.25-3.03 (m, 1H), 2.95-2.84 (m, 1H), 2.66-2.63 (m, 1H), 2.35-2.23 (m, 1H), 1.87-1.82 (m, 4H), 1.80-1.75 (m, 1H), 1.66-1.61 (m, 5H), 1.30-1.28 (m, 3H), 1.07-1.04 (m, 3H), 0.99-0.91 (m, 2H), 0.87-0.84 (m, 1H). m/z: ES⁺ [M+H]⁺=551.25.

### Example 3.53: N-(5-fluoro-2-((5-((1S,5R)-2-((3aR,6aR)-octahydrocyclopenta[c]pyrrole-3a-carbonyl)-2,6-diazabicyclo[3.2.1]octan-6-yl)-1,2,4-triazin-6-yl)oxy)phenyl)-N-isopropylcyclopropane carboxamide

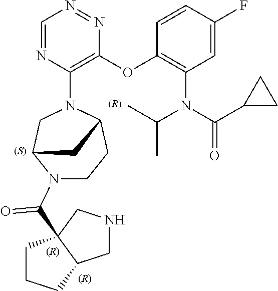

Off-white solid, HCl salt (61 mg, 30% over 2 steps)

¹H NMR (400 MHz, DMSO-d₆, 300 K) δ 10.14-9.72 (m, 1H), 9.71-9.10 (m, 1H), 9.08-8.75 (m, 1H), 7.91-7.61 (m, 1H), 7.60-6.87 (m, 2H), 5.38-5.10 (m, 1H), 5.08-4.47 (m, 2H), 4.46-4.07 (m, 1H), 4.04-3.83 (m, 1H), 3.72-3.55 (m, 1H), 3.42-3.27 (m, 2H), 3.25-3.05 (m, 1H), 3.01-2.85 (m, 1H), 2.17-1.95 (m, 3H), 1.93-1.88 (m, 2H), 1.82-1.67 (m, 3H), 1.62-1.37 (m, 2H), 1.27-1.04 (m, 5H), 1.01-0.80 (m, 3H), 0.77-0.39 (m, 5H). m/z: ES⁺ [M+H]⁺=564.30.

### Example 3.54: N-cyclopropyl-N-(5-fluoro-2-((5-((1S,5R)-2-((3aR,6aR)-octahydrocyclopenta[c]pyrrole-3a-carbonyl)-2,6-diazabicyclo[3.2.1]octan-6-yl)-1,2,4-triazin-6-yl)oxy)phenyl)cyclopropane carboxamide

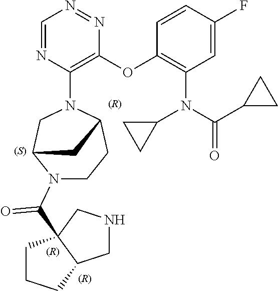

Colourless solid, HCl salt (53 mg, 39% over 2 steps).

¹H NMR (400 MHz, DMSO-d₆, 300 K) δ 9.82 (br s, 1H), 9.47 (br s, 1H), 9.05-8.70 (m, 1H), 7.91-6.97 (m, 3H), 5.43-4.84 (m, 2H), 4.83-3.75 (m, 4H), 3.68-3.36 (m, 4H), 3.30-3.11 (m, 2H), 3.01-2.83 (m, 1H), 2.22-1.69 (m, 8H), 1.66-1.32 (m, 2H), 1.11-0.12 (m, 8H). m/z: ES⁺ [M+H]⁺=562.30.

### Example 3.55: ((1S,5R)-6-(6-(4-fluoro-2-(2-isopropyl-5-methyl-1H-imidazol-1-yl)phenoxy)-1,2,4-triazin-5-yl)-2,6-diazabicyclo[3.2.1]octan-2-yl)(tetrahydro-1H-pyrrolizin-7a(5H)-yl)methanone

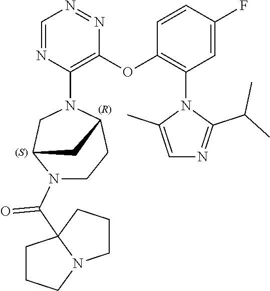

Colourless solid (29 mg, 34%).

¹H NMR (400 MHz, DMSO-d₆, 300 K) δ 8.54-8.45 (m, 1H), 7.69-7.50 (m, 3H), 6.75-6.45 (m, 1H), 5.43-5.02 (m, 1H), 4.98-4.13 (m, 2H), 3.98-3.60 (m, 1H), 3.52-3.40 (m, 2H), 3.05-2.91 (m, 2H), 2.61-2.52 (m, 3H), 2.19-2.13 (m, 2H), 1.92-1.75 (m, 9H), 1.63-1.55 (m, 4H), 1.07-0.85 (m, 6H). m/z: ES⁺ [M+H]⁺=561.30.

### Example 3.56: ((1S,5R)-6-(6-(2-(4-cyclopropyl-6-methylpyrimidin-5-yl)-4-fluorophenoxy)-1,2,4-triazin-5-yl)-2,6-diazabicyclo[3.2.1]octan-2-yl)((3aR,6aS)-6a-fluorohexahydrocyclopenta[c]pyrrol-3a(1H)-yl)methanone and ((1S,5R)-6-(6-(2-(4-cyclopropyl-6-methylpyrimidin-5-yl)-4-fluorophenoxy)-1,2,4-triazin-5-yl)-2,6-diazabicyclo[3.2.1]octan-2-yl)((3aS,6aR)-6a-fluorohexahydrocyclopenta[c]pyrrol-3a(1H)-yl)methanone

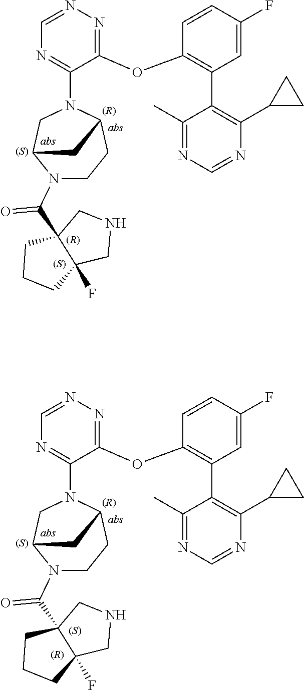

Colourless solid (34 mg, 19% over 2 steps).

¹H NMR (400 MHz, DMSO-d₆, 300 K) δ 8.90-8.63 (m, 1H), 8.63-8.22 (m, 1H), 7.78-7.58 (m, 1H), 7.58-7.23 (m, 2H), 5.30-4.32 (m, 2H), 4.23-3.31 (m, 6H), 3.01-2.58 (m, 3H), 2.49-2.15 (m, 5H), 2.14-1.79 (m, 4H), 1.79-1.31 (m, 5H), 1.15-0.48 (m, 4H). m/z: ES⁺ [M+H]⁺=561.30.

### Examples 3.57 and 3.58: atropisomers of ((1S,5R)-6-(6-(2-(4-cyclopropyl-6-methylpyrimidin-5-yl)-4-fluorophenoxy)-1,2,4-triazin-5-yl)-2,6-diazabicyclo[3.2.1]octan-2-yl)((3aR,6aR)-hexahydrocyclopenta[c]pyrrol-3a(1H)-yl)methanone

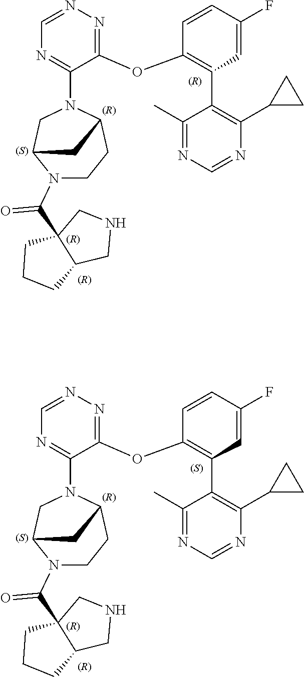

Mixture isolated as a colourless solid (46 mg, 53% over 2 steps).

¹H NMR (400 MHz, DMSO-d₆, 300 K) δ 8.91-8.63 (m, 1H), 8.63-8.22 (m, 1H), 7.78-7.56 (m, 1H), 7.56-7.38 (m, 2H), 5.38-4.06 (m, 2H), 4.06-3.48 (m, 2H), 3.39-3.28 (m, 2H), 3.11-2.76 (m, 3H), 2.48-2.27 (m, 2H), 2.26-2.01 (m, 5H), 2.00-1.27 (m, 10H), 1.04-0.85 (m, 3H), 0.85-0.48 (m, 1H). m/z: ES⁺ [M+H]⁺=571.15.

Separation by chiral HPLC gave two single stable atropisomers:

Peak #1: Rt1=10.73 min, colourless solid (46 mg, 54%).

¹H NMR (400 MHz, DMSO-d₆, 300 K) δ 8.88-8.63 (m, 1H), 8.63-8.22 (m, 1H), 7.78-7.56 (m, 1H), 7.56-7.28 (m, 2H), 5.38-4.26 (m, 2H), 4.19-3.68 (m, 2H), 3.68-3.42 (m, 1H), 3.31-3.23 (m, 1H), 3.11-2.72 (m, 4H), 2.51-2.26 (m, 2H), 2.25-2.11 (m, 3H), 1.97-1.73 (m, 4H), 1.73-1.58 (m, 4H), 1.58-1.23 (m, 3H), 1.01-0.25 (m, 4H). m/z: ES⁺ [M+H]⁺=571.25.

Peak #2: Rt2=21.37 min, colourless solid (61 mg, 71%).

¹H NMR (400 MHz, DMSO-d₆, 300 K) δ 8.88-8.63 (m, 1H), 8.63-8.22 (m, 1H), 7.78-7.56 (m, 1H), 7.56-7.28 (m, 2H), 5.38-4.31 (m, 2H), 4.22-3.88 (m, 1H), 3.88-3.69 (m, 1H), 3.52-3.47 (m, 1H), 3.35-3.28 (m, 1H), 3.14-2.91 (m, 2H), 2.91-2.77 (m, 1H), 2.71-2.58 (m, 1H), 2.51-2.47 (m, 1H), 2.21-2.09 (m, 3H), 2.01-1.79 (m, 4H), 1.79-1.58 (m, 5H), 1.58-1.42 (m, 2H), 1.42-1.33 (m, 1H), 1.02-0.88 (m, 3H), 0.88-0.65 (m, 1H). m/z: ES⁺ [M+H]⁺=571.20.

### Example 3.59: N-ethyl-N-(5-fluoro-2-((5-((1S,5R)-2-((3aR,6aR)-2-methyloctahydrocyclopenta[c]pyrrole-3a-carbonyl)-2,6-diazabicyclo[3.2.1]octan-6-yl)-1,2,4-triazin-6-yl)oxy)phenyl)isobutyramide

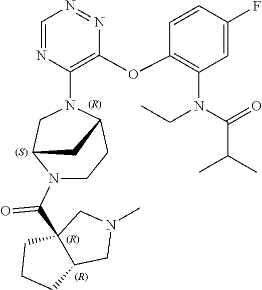

Colourless solid, formic acid salt (34 mg, 15%) as a colourless solid.

¹H NMR (400 MHz, DMSO-d₆, 300K) δ 8.63-8.45 (m, 1H), 8.17 (s, 1H), 7.65-7.23 (m, 3H), 5.30-4.93 (m, 1H), 4.92-4.60 (m, 1H), 4.33-3.49 (m, 5H), 3.34-3.04 (m, 2H), 2.95-2.67 (m, 2H), 2.66-2.55 (m, 1H), 2.47-2.32 (m, 2H), 2.32-2.12 (m, 4H), 2.09-1.54 (m, 8H), 1.54-1.33 (m, 2H), 1.08-0.88 (m, 6H), 0.81-0.57 (m, 3H). m/z: ES⁺ [M+H]⁺=566.25.

### Example 3.60: ((1S,5R)-6-(6-(2-(4-cyclopropyl-6-methylpyrimidin-5-yl)-4-fluorophenoxy)-1,2,4-triazin-5-yl)-2,6-diazabicyclo[3.2.1]octan-2-yl)((S)-2-ethylpyrrolidin-2-yl)methanone

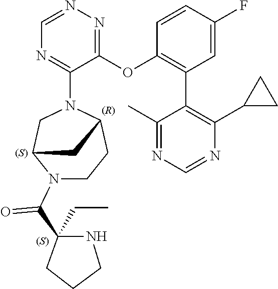

Colourless solid, TFA salt (12 mg, 16% over 2 steps).

¹H NMR (400 MHz, DMSO-d₆, 300 K) δ 8.91-8.79 (m, 2H), 7.78-7.56 (m, 1H), 7.56-7.41 (m, 2H), 5.38-4.72 (m, 2H), 4.41-3.89 (m, 2H), 3.81-3.68 (m, 2H), 3.42-2.88 (m, 3H), 2.46-2.09 (m, 6H), 2.08-1.75 (m, 6H), 1.74-1.39 (m, 2H), 1.12-0.77 (m, 7H). m/z: ES⁺ [M+H]⁺=559.35.

### Examples 3.61 and 3.62: N-ethyl-5-fluoro-N-isopropyl-2-((5-((1S,5R)-2-((3aS,6aS)-octahydrocyclopenta[c]pyrrole-3a-carbonyl)-2,6-diazabicyclo[3.2.1]octan-6-yl)-1,2,4-triazin-6-yl)oxy)benzamide and N-ethyl-5-fluoro-N-isopropyl-2-((5-((1S,5R)-2-((3aR,6aR)-octahydrocyclopenta[c]pyrrole-3a-carbonyl)-2,6-diazabicyclo[3.2.1]octan-6-yl)-1,2,4-triazin-6-yl)oxy)benzamide

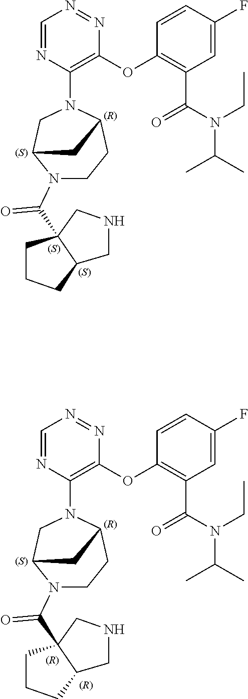

Separation by chiral HPLC gave two single stereoisomers:

Peak #1: colourless solid (28 mg, 4% over 2 steps).

¹H NMR (400 MHz, DMSO-d₆, 300 K) δ 8.50-8.47 (m, 1H), 7.47-7.46 (m, 1H), 7.39-7.34 (m, 2H), 5.45-5.11 (m, 1H), 4.85-4.63 (m, 1H), 4.37-3.95 (m, 2H), 3.93-3.57 (m, 3H), 3.47-3.35 (m, 2H), 3.28-3.01 (m, 1H), 2.97-2.83 (m, 2H), 2.79-2.63 (m, 1H), 2.45-2.38 (m, 1H), 2.19-1.53 (m, 9H), 1.50-1.39 (m, 1H), 1.38-1.26 (m, 1H), 1.19-1.03 (m, 4H), 1.02-0.92 (m, 3H), 0.86-0.53 (m, 2H). m/z: ES⁺ [M+H]⁺=522.30.

Peak #2: colourless solid (30 mg, 4% over 2 steps).

¹H NMR (400 MHz, DMSO-d₆, 300 K) δ 8.56-8.47 (m, 1H), 7.47-7.46 (m, 1H), 7.39-7.34 (m, 2H), 5.47-5.08 (m, 1H), 4.83-4.68 (m, 1H), 4.37-3.90 (m, 2H), 3.93-3.51 (m, 3H), 3.42-3.28 (m, 3H), 3.18-2.84 (m, 3H), 2.83-2.69 (m, 1H), 2.45-2.38 (m, 1H), 2.29-1.63 (m, 9H), 1.61-1.51 (m, 1H), 1.50-1.25 (m, 1H), 1.17-1.03 (m, 3H), 1.02-0.89 (m, 3H), 0.86-0.53 (m, 2H). m/z: ES⁺ [M+H]⁺=522.30.

### Example 3.63: N-ethyl-2-((5-((1S,5R)-2-((S)-2-ethylpyrrolidine-2-carbonyl)-2,6-diazabicyclo[3.2.1]octan-6-yl)-1,2,4-triazin-6-yl)oxy)-5-fluoro-N-isopropylbenzamide

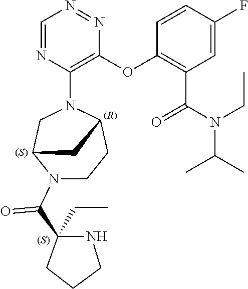

White solid (68 mg, 34% over 2 steps).

¹H NMR (400 MHz, DMSO-d₆, 300 K) 8.53-8.50 (m, 1H), 7.50-7.27 (m, 3H), 5.40-5.29 (m, 1H), 5.20-4.80 (m, 1H), 4.45-4.33 (m, 1H), 4.22-3.76 (m, 2H), 3.86-3.45 (m, 2H), 3.25-3.07 (m, 2H), 2.90-2.80 (m, 1H), 2.63-2.60 (m, 1H), 2.16-1.97 (m, 3H), 1.80-1.61 (m, 7H), 1.07-1.04 (m, 4H), 0.98-0.92 (m, 3H), 0.79-0.72 (m, 4H), 0.72-0.66 (m, 2H). m/z: ES⁺ [M+H]⁺=540.30.

### Example 3.64: ((1S,5R)-6-(6-(2-(4-cyclopropyl-1-methyl-1H-pyrazol-5-yl)-4-fluorophenoxy)-1,2,4-triazin-5-yl)-2,6-diazabicyclo[3.2.1]octan-2-yl)((3aR,6aR)-hexahydrocyclopenta[c]pyrrol-3a(1H)-yl)methanone

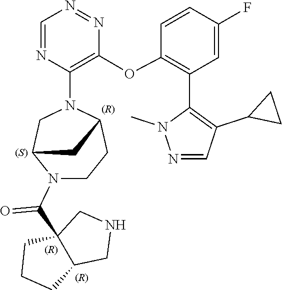

Off-white solid, formic acid salt (34 mg, 18% over 2 steps).

¹H NMR (400 MHz, DMSO-d₆, 300 K) δ 8.46-8.28 (m, 2H), 7.61-6.98 (m, 4H), 5.13-4.95 (m, 1H), 4.89-4.60 (m, 1H), 4.45-3.78 (m, 2H), 3.57-3.51 (m, 5H), 3.21-3.08 (m, 2H), 2.94-2.82 (m, 1H), 2.61-2.42 (m, 2H), 1.91-1.82 (m, 4H), 1.84-1.62 (m, 4H), 1.60-1.24 (m, 4H), 0.65-0.29 (m, 4H). m/z: ES⁺ [M+H]⁺=559.25.

### Example 3.65: N-(5-fluoro-2-((5-((1S,5R)-2-((3aR,6aR)-octahydrocyclopenta[c]pyrrole-3a-carbonyl)-2,6-diazabicyclo[3.2.1]octan-6-yl)-1,2,4-triazin-6-yl)oxy)phenyl)-N-isopropylisobutyramide

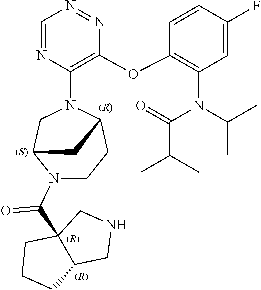

Colourless solid (37 mg, 23% over 2 steps).

¹H NMR (400 MHz, DMSO-d₆, 300 K) δ 8.62-8.48 (m, 1H), 8.28 (s, 1H), 7.70-7.59 (m, 1H), 7.47-7.33 (m, 2H), 5.18-4.64 (m, 3H), 4.55-4.49 (m, 1H), 4.33-3.77 (m, 3H), 3.71-3.56 (m, 2H), 3.30-3.10 (m, 2H), 3.00-2.94 (m, 1H), 2.63-2.58 (m, 1H), 2.39-2.24 (m, 1H), 2.02-1.67 (m, 8H), 1.49-1.36 (m, 2H), 1.14-1.10 (m, 3H), 0.94-0.87 (m, 6H), 0.68-0.50 (m, 3H). m/z: ES⁺ [M+H]⁺=566.25.

### Example 3.66: ((1S,5R)-6-(6-(4-fluoro-2-(4-isopropyl-1-methyl-1H-pyrazol-5-yl)phenoxy)-1,2,4-triazin-5-yl)-2,6-diazabicyclo[3.2.1]octan-2-yl)((3aR,6aR)-hexahydrocyclopenta[c]pyrrol-3a(1H)-yl)methanone

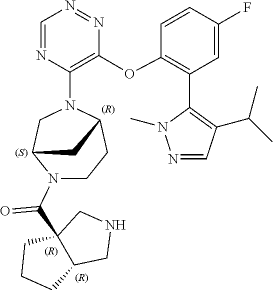

Off-white solid, formic acid salt (44 mg, 22% over 2 steps).

¹H NMR (400 MHz, DMSO-d₆, 300 K) δ 8.53-8.35 (m, 1H), 8.31 (s, 1H), 7.62-7.15 (m, 4H), 5.12-4.95 (m, 1H), 4.90-4.55 (m, 2H), 4.20-3.86 (m, 2H), 3.76-2.96 (m, 7H), 2.63-2.52 (m, 2H), 2.05-1.85 (m, 4H), 1.75-1.52 (m, 4H), 1.50-1.39 (m, 4H), 1.02-0.95 (m, 3H), 0.88-0.75 (m, 3H). m/z: ES⁺ [M+H]⁺=561.30.

### Example 3.67: ((1S,5R)-6-(6-(2-(4-cyclopropyl-6-methylpyrimidin-5-yl)-4-fluorophenoxy)-1,2,4-triazin-5-yl)-2,6-diazabicyclo[3.2.1]octan-2-yl)((3aR,6aR)-2-methylhexahydrocyclopenta[c]pyrrol-3a(1H)-yl)methanone

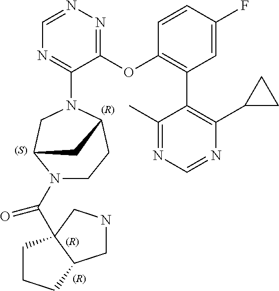

Colourless solid (43 mg, 32%).

¹H NMR (400 MHz, DMSO-d₆, 300K) δ 8.89-8.63 (m, 1H), 8.52-8.38 (m, 1H), 7.76-7.52 (m, 1H), 7.52-7.38 (m, 2H), 5.34-4.38 (m, 2H), 4.38-3.49 (m, 3H), 3.22-2.58 (m, 2H), 2.48-2.29 (m, 2H), 2.22-2.11 (m, 7H), 1.98-1.32 (m, 12H), 1.01-0.88 (m, 3H), 0.85-0.54 (m, 1H). m/z: ES⁺ [M+H]⁺=585.15.

### Examples 3.68 and 3.69: atropisomers of ((S)-2-ethylpyrrolidin-2-yl)((1S,5R)-6-(6-(4-fluoro-2-(2-isopropyl-5-methyl-1H-imidazol-1-yl)phenoxy)-1,2,4-triazin-5-yl)-2,6-diazabicyclo[3.2.1]octan-2-yl)methanone

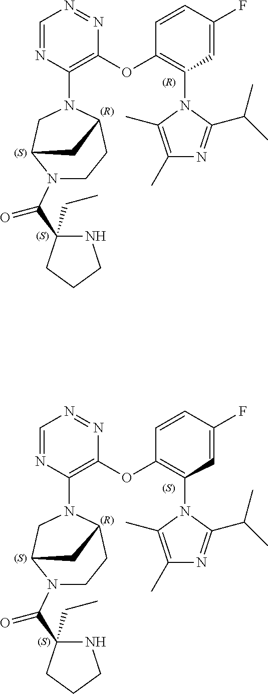

Mixture isolated as an off-white solid (50 mg, 33% over 2 steps).

m/z: ES⁺ [M+H]⁺=549.20.

Separation by chiral HPLC gave two single stable atropisomers:

Peak #1: Rt1=6.35 min, off-white solid (4 mg, 14%).

¹H NMR (400 MHz, DMSO-d₆, 300 K) δ 8.49-8.31 (m, 1H), 7.82-7.45 (m, 3H), 6.56 (s, 1H), 5.51-5.10 (m, 1H), 4.72-4.54 (m, 1H), 4.42-4.10 (m, 1H), 3.77-3.51 (m, 2H), 3.20-2.63 (m, 4H), 2.20-2.00 (m, 1H), 1.94-1.78 (m, 5H), 1.75-1.61 (m, 8H), 1.10-0.92 (m, 6H), 0.90-0.78 (m, 3H). m/z: ES⁺ [M+H]⁺=549.25.

Peak #2: Rt2=7.83 min, light yellow solid (7 mg, 24%).

¹H NMR (400 MHz, DMSO-d₆, 300 K) δ 8.49-8.05 (m, 1H), 7.82-7.45 (m, 3H), 6.82-6.56 (m, 1H), 5.51-4.92 (m, 1H), 4.72-4.54 (m, 1H), 4.42-4.10 (m, 1H), 3.77-3.51 (m, 2H), 3.20-2.63 (m, 4H), 2.20-2.15 (m, 1H), 1.94-1.81 (m, 10H), 1.80-1.61 (m, 2H), 1.15-1.10 (m, 1H), 1.08-0.95 (m, 3H), 0.90-0.78 (m, 6H). m/z: ES⁺ [M+H]⁺=549.25.

### Examples 3.70 and 3.71: atropisomers of ((1S,5R)-6-(6-(4-fluoro-2-(2-isopropyl-4,5-dimethyl-1H-imidazol-1-yl)phenoxy)-1,2,4-triazin-5-yl)-2,6-diazabicyclo[3.2.1]octan-2-yl)((3aR,6aR)-hexahydrocyclopenta[c]pyrrol-3a(1H)-yl)methanone

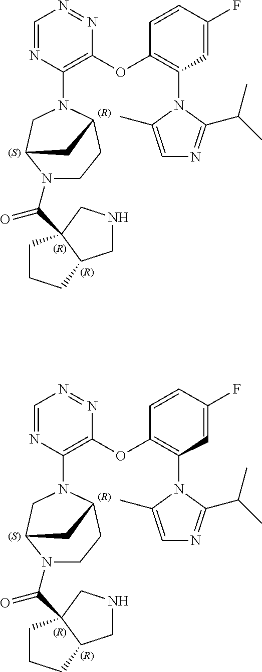

Mixture isolated as a colourless solid, bis(formic acid) salt (117 mg, 40% over 2 steps).

¹H NMR (400 MHz, DMSO-d₆, 300 K) δ 8.56-8.44 (m, 1H), 8.31 (s, 2H), 7.72-7.48 (m, 3H), 5.25-5.14 (m, 1H), 4.85-4.73 (m, 1H), 4.64-4.23 (m, 2H), 3.85-3.78 (m, 1H), 3.65-3.55 (m, 1H), 3.50-3.41 (m, 1H), 3.34-3.32 (m, 2H), 3.15-3.08 (m, 2H), 2.78 (m, 1H), 2.57-2.56 (m, 1H), 2.06-1.88 (m, 5H), 1.85-1.75 (m, 4H), 1.75-1.72 (m, 3H), 1.62-1.35 (m, 3H), 1.15-1.05 (m, 2H), 1.03-0.95 (m, 2H), 0.92-0.74 (m, 3H). m/z: ES⁺ [M+H]⁺=575.30.

Separation by chiral HPLC gave two single stable atropisomers:

Peak #1: Rt1=7.93 min, colourless solid (22 mg, 20%).

¹H NMR (400 MHz, DMSO-d₆, 300 K) δ 8.52-8.48 (m, 1H), 8.32 (s, 1H), 7.67-7.64 (m, 1H), 7.59-7.50 (m, 2H), 5.30-5.14 (m, 1H), 4.74-4.71 (m, 1H), 4.57-4.04 (m, 1H), 3.90-3.78 (m, 1H), 3.66-3.45 (m, 2H), 3.29-3.23 (m, 1H), 3.16-2.81 (m, 3H), 2.69-2.57 (m, 2H), 1.97-1.92 (m, 5H), 1.84-1.45 (m, 8H), 1.43-1.31 (m, 2H), 1.17-1.15 (m, 2H), 0.99-0.96 (m, 3H), 0.90-0.87 (m, 3H). m/z: ES⁺ [M+H]⁺=575.30.

Peak #2: Rt=14.33 min, colourless solid (10 mg, 8%).

¹H NMR (400 MHz, DMSO-d₆, 300 K) δ 8.53-8.49 (m, 1H), 8.27-8.23 (m, 2H), 7.73-7.66 (m, 1H), 7.62-7.60 (m, 1H), 7.56-7.52 (m, 1H), 5.12-4.85 (m, 2H), 4.82-4.64 (m, 1H), 4.62-4.34 (m, 1H), 4.22-3.87 (m, 1H), 3.68-2.58 (m, 2H), 3.21-3.11 (m, 2H), 2.96-2.92 (m, 1H), 2.65-2.45 (m, 3H), 2.10-1.92 (m, 2H), 1.89-1.82 (m, 3H), 1.80-1.75 (m, 5H), 1.74-1.58 (m, 4H), 1.58-1.38 (m, 2H), 1.10-0.95 (m, 3H), 0.84-0.77 (m, 1H), 0.72-0.67 (m, 2H). m/z: ES⁺ [M+H]⁺=575.30.

### Example 3.72 ((1S,3S,5S)-2-azabicyclo[3.1.0]hexan-3-yl)((1S,5R)-6-(6-((MP)-2-(2-cyclopropyl-5-methyl-1H-imidazol-1-yl)-4-fluorophenoxy)-1,2,4-triazin-5-yl)-2,6-diazabicyclo[3.2.1]octan-2-yl)methanone

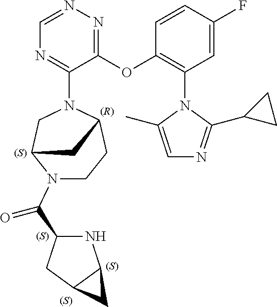

Colourless solid (33 mg, 22% over 2 steps).

¹H NMR (400 MHz, DMSO-d₆, 300 K) δ 8.69-8.28 (m, 1H), 7.89-7.60 (m, 2H), 7.59-7.42 (m, 1H), 6.65-6.25 (m, 1H), 5.27-4.79 (m, 1H), 4.78-4.55 (m, 1H), 4.49-3.81 (m, 2H), 3.80-3.48 (m, 2H), 3.12-2.79 (m, 1H), 2.66-2.58 (m, 1H), 2.47-2.38 (m, 1H), 2.01-1.86 (m, 3H), 1.85-1.71 (m, 3H), 1.70-1.51 (m, 2H), 1.50-1.21 (m, 2H), 1.05-0.63 (m, 3H), 0.62-0.35 (m, 3H), 0.30-0.15 (m, 1H). m/z: ES⁺ [M+H]⁺=531.25.

### Example 3.73 ((1S,5R)-6-(6-(2-(2-cyclopropyl-5-methyl-1H-imidazol-1-yl)-4-fluorophenoxy)-1,2,4-triazin-5-yl)-2,6-diazabicyclo[3.2.1]octan-2-yl)((1S,2S,5R)-2-methyl-3-azabicyclo[3.1.0]hexan-2-yl)methanone and ((1S,5R)-6-(6-(2-(2-cyclopropyl-5-methyl-1H-imidazol-1-yl)-4-fluorophenoxy)-1,2,4-triazin-5-yl)-2,6-diazabicyclo[3.2.1]octan-2-yl)((1S,2R,5R)-2-methyl-3-azabicyclo[3.1.0]hexan-2-yl)methanone

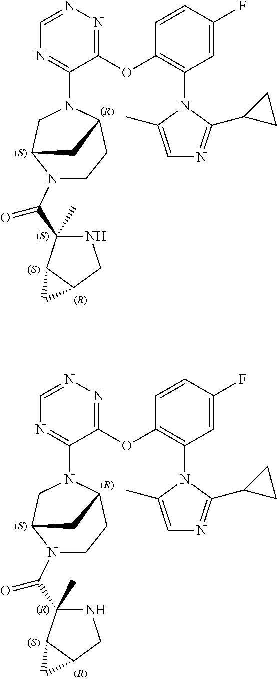

Mixture isolated as a colourless solid, formic acid salt (14 mg, 8% over 2 steps).

¹H NMR (400 MHz, DMSO-d₆, 295 K) δ 8.55-8.43 (m, 1H), 8.16 (s, 1H), 7.73-7.55 (m, 3H), 6.54-6.47 (m, 1H), 5.21-5.05 (m, 1H), 4.83-4.73 (m, 1H), 4.47-4.28 (m, 1H), 3.92-3.90 (m, 1H), 3.77-3.74 (m, 1H), 3.56-3.48 (m, 2H), 2.82-2.75 (m, 1H), 2.63-2.58 (m, 1H), 1.98-1.95 (m, 1H), 1.92-1.82 (m, 4H), 1.58-1.29 (m, 8H), 0.77-0.24 (m, 6H). m/z: ES⁺ [M+H]⁺=545.25.

### Example 3.74 ((1S,5R)-6-(6-((MP)-2-(2-cyclopropyl-5-methyl-1H-imidazol-1-yl)-4-fluorophenoxy)-1,2,4-triazin-5-yl)-2,6-diazabicyclo[3.2.1]octan-2-yl)((1R,2S,5S)-2-methyl-3-azabicyclo[3.1.0]hexan-2-yl)methanone and ((1S,5R)-6-(6-((MP)-2-(2-cyclopropyl-5-methyl-1H-imidazol-1-yl)-4-fluorophenoxy)-1,2,4-triazin-5-yl)-2,6-diazabicyclo[3.2.1]octan-2-yl)((1R,2R,5S)-2-methyl-3-azabicyclo[3.1.0]hexan-2-yl)methanone

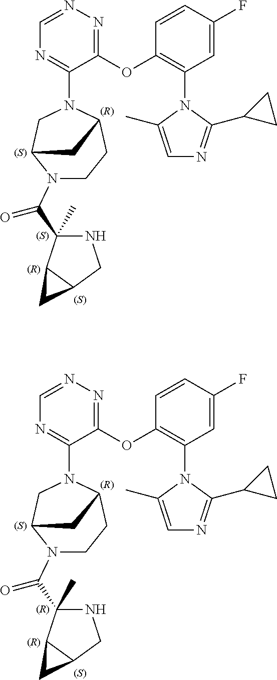

Mixture isolated as a colourless solid, formic acid salt (16 mg, 9% over 2 steps).

¹H NMR (400 MHz, DMSO-d₆, 295 K) δ 8.63-8.38 (m, 1H), 8.18 (s, 2H), 7.89-7.45 (m, 3H), 6.78-6.30 (m, 1H), 6.00-5.20 (m, 1H), 4.88-4.70 (m, 1H), 4.60-4.40 (m, 1H), 3.95-3.90 (m, 1H), 3.77-3.72 (m, 1H), 3.52-3.45 (m, 1H), 2.93-2.80 (m, 2H), 2.80-2.60 (m, 1H), 2.12-1.98 (m, 1H), 1.97-1.79 (m, 4H), 1.79-1.51 (m, 3H), 1.49-1.39 (m, 1H), 1.39-1.25 (m, 4H), 0.80-0.54 (m, 3H), 0.52-0.29 (m, 3H). m/z: ES⁺ [M+H]⁺=545.25.

### Example 3.75 ((1R,5R)-3-azabicyclo[3.1.0]hexan-1-yl)((1S,5R)-6-(6-(2-(2-cyclopropyl-5-methyl-1H-imidazol-1-yl)-4-fluorophenoxy)-1,2,4-triazin-5-yl)-2,6-diazabicyclo[3.2.1]octan-2-yl)methanone and ((1S,5S)-3-azabicyclo[3.1.0]hexan-1-yl)((1S,5R)-6-(6-(2-(2-cyclopropyl-5-methyl-1H-imidazol-1-yl)-4-fluorophenoxy)-1,2,4-triazin-5-yl)-2,6-diazabicyclo[3.2.1]octan-2-yl)methanone

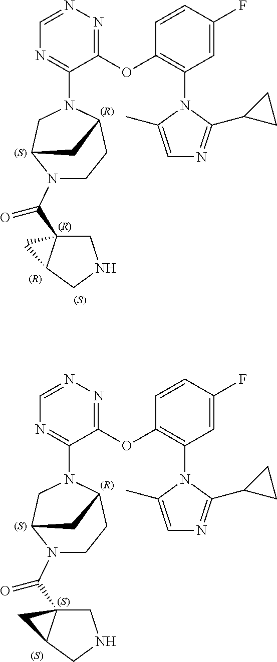

Mixture isolated as a colourless solid (14 mg, 11% over 2 steps).

¹H NMR (400 MHz, DMSO-d₆, 295 K) δ 8.14 (s, 1H), 7.92-7.86 (m, 1H), 7.62-7.53 (m, 1H), 7.45-7.33 (m, 1H), 7.31-7.06 (m, 1H), 6.65-6.48 (m, 1H), 5.12-4.73 (m, 1H), 4.72-4.35 (m, 1H), 4.17-3.82 (m, 1H), 3.78-3.62 (m, 1H), 3.50-3.37 (m, 1H), 3.09-2.83 (m, 1H), 2.82-2.52 (m, 3H), 2.42-2.31 (m, 2H), 2.09-1.91 (m, 3H), 1.87-1.75 (m, 1H), 1.74-1.47 (m, 5H), 0.91-0.65 (m, 6H). m/z: ES⁺ [M+H]⁺=530.25.

### Example 3.76 ((1S,5R)-6-(6-(2-(2,5-dicyclopropyl-1H-imidazol-1-yl)-4-fluorophenoxy)-1,2,4-triazin-5-yl)-2,6-diazabicyclo[3.2.1]octan-2-yl)(tetrahydro-1H-pyrrolizin-7a(5H)-yl)methanone

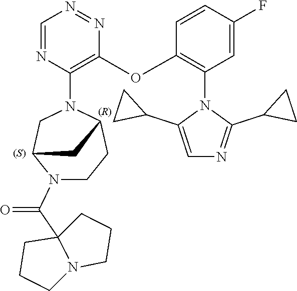

Yellow solid (7 mg, 18%).

¹H NMR (400 MHz, DMSO-d₆, 295 K) δ 8.53-8.45 (m, 1H), 8.27 (s, 2H), 7.80-7.65 (m, 2H), 7.65-7.50 (m, 1H), 6.50-6.30 (m, 1H), 5.20-5.00 (m, 1H), 4.80-4.70 (m, 1H), 4.50-4.40 (m, 1H), 4.25-4.15 (m, 1H), 3.80-3.52 (m, 3H), 3.30-3.20 (m, 2H), 3.10-2.90 (m, 2H), 2.49-2.19 (m, 1H), 2.19-2.06 (m, 2H), 2.06-1.96 (m, 2H), 1.96-1.62 (m, 5H), 1.60-1.39 (m, 2H), 0.85-0.60 (m, 4H), 0.60-0.30 (m, 4H). m/z: ES⁺ [M+H]⁺=585.30.

### Examples 3.77 and 3.78: atropisomers of ((1S,5R)-6-(6-(2-(2-cyclopropyl-4,5-dimethyl-1H-imidazol-1-yl)-4-fluorophenoxy)-1,2,4-triazin-5-yl)-2,6-diazabicyclo[3.2.1]octan-2-yl)((S)-2-ethylpyrrolidin-2-yl)methanone

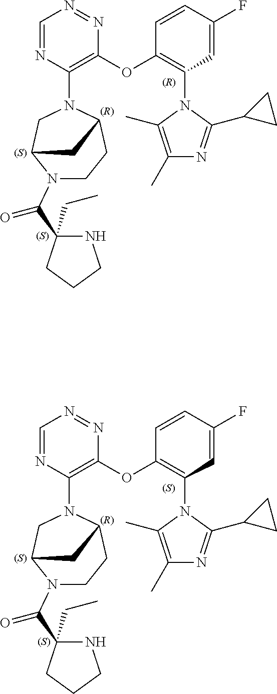

Separation by chiral HPLC gave two single stable atropisomers:

Peak #1: Rt=10.02 min, colourless solid (1.5 mg, 2%).

¹H NMR (400 MHz, CD₃OD, 296 K) δ 8.54-8.53 (m, 1H), 7.68-7.41 (m, 3H), 5.44-4.85 (m, 2H), 4.24-3.67 (m, 3H), 3.56-3.24 (m, 2H), 3.07-2.96 (m, 1H), 2.55-2.36 (m, 1H), 2.24-1.96 (m, 12H), 1.91-1.80 (m, 4H), 1.20-0.96 (m, 4H), 0.94-0.88 (m, 3H). m/z: ES⁺ [M+H]⁺=561.30.

Peak #2: Rt=16.72 min, colourless solid (2.5 mg, 4%).

¹H NMR (400 MHz, DMSO-d₆, 296 K) δ 8.54-8.47 (m, 1H), 7.72-7.68 (m, 1H), 7.60-7.51 (m, 2H), 5.55-5.23 (m, 1H), 4.34-4.22 (m, 2H), 4.02-3.54 (m, 2H), 3.13-2.82 (m, 2H), 2.68-2.61 (m, 1H), 2.35-2.15 (m, 1H), 1.96-1.86 (m, 4H), 1.86-1.79 (m, 3H), 1.79-1.50 (m, 8H), 1.45-1.32 (m, 1H), 0.82-0.75 (m, 3H), 0.71-0.65 (m, 2H), 0.55-0.34 (m, 2H). m/z: ES⁺ [M+H]⁺=561.30.

### Examples 3.79 and 3.80: atropisomers of 6-(5-fluoro-2-((5-((1S,5R)-2-((S)-2-methylpyrrolidine-2-carbonyl)-2,6-diazabicyclo[3.2.1]octan-6-yl)-1,2,4-triazin-6-yl)oxy)phenyl)-5-isopropyl-1-methylpyridin-2(1H)-one

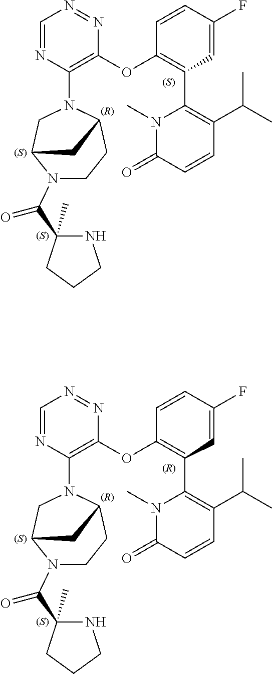

Mixture isolated as a colourless solid, formic acid salt (20 mg, 8% over 2 steps).

¹H NMR (400 MHz, DMSO-d₆, 296 K) δ 8.80-8.45 (m, 1H), 8.31 (s, 1H), 7.80-7.58 (m, 1H), 7.55-7.30 (m, 3H), 6.60-6.35 (m, 1H), 5.75-4.95 (m, 2H), 4.90-4.60 (m, 1H), 4.50-3.90 (m, 2H), 3.85-3.70 (m, 1H), 3.69-3.50 (m, 1H), 3.07 (s, 3H), 3.00-2.70 (m, 1H), 2.68-2.45 (m, 1H), 2.35-2.10 (m, 2H), 2.05-1.45 (m, 7H), 2.40-1.15 (m, 3H), 1.05-0.90 (m, 3H), 0.80-0.50 (m, 3H). m/z: ES⁺ [M+H]⁺=562.25.

Separation by chiral HPLC gave two single stable atropisomers:

Peak #1: Rt=3.94 min, colourless solid (4 mg, 35%).

¹H NMR (400 MHz, DMSO-d₆, 293 K) δ 8.68-8.17 (m, 3H), 7.68-7.41 (m, 2H), 7.67-7.31 (m, 1H), 7.31-7.10 (m, 1H), 6.70-6.35 (m, 1H), 5.41-5.04 (m, 1H), 4.73-4.34 (m, 2H), 4.34-4.02 (m, 1H), 3.98-3.54 (m, 2H), 3.48-3.26 (m, 1H), 3.20-3.15 (m, 4H), 3.25-3.17 (m, 1H), 2.97-2.76 (m, 1H), 2.46-1.80 (m, 7H), 1.80-1.43 (m, 5H), 1.08-1.85 (m, 3H), 0.78-0.41 (m, 3H). m/z: ES⁺ [M+H]⁺=562.30.

Peak #2: Rt=6.27 min, colourless solid (4 mg, 35%).

¹H NMR (400 MHz, DMSO-d₆, 293 K) δ 8.61-8.32 (m, 2H), 7.72-7.50 (m, 2H), 7.50-7.37 (m, 1H), 7.37-7.28 (m, 1H), 6.85-6.45 (m, 1H), 5.39-5.12 (m, 1H), 5.12-4.99 (m, 1H), 4.82-4.16 (m, 1H), 4.12-3.54 (m, 3H), 3.29-3.21 (m, 4H), 3.15-3.10 (m, 1H), 2.62-2.50 (m, 1H), 2.45-2.25 (m, 1H), 2.23-1.98 (m, 4H), 1.95-1.85 (m, 1H), 1.83-1.70 (m, 1H), 1.65-1.55 (m, 3H), 1.38-1.15 (m, 2H), 1.12-0.95 (m, 3H), 0.92-0.65 (m, 3H).

m/z: ES⁺ [M+H]⁺=562.30.

### Example 3.81 ((1S,5R)-6-(6-(4-fluoro-2-(4-isopropyl-1-methyl-1H-1,2,3-triazol-5-yl)phenoxy)-1,2,4-triazin-5-yl)-2,6-diazabicyclo[3.2.1]octan-2-yl)((3aR,6aR)-hexahydrocyclopenta[c]pyrrol-3a(1H)-yl)methanone

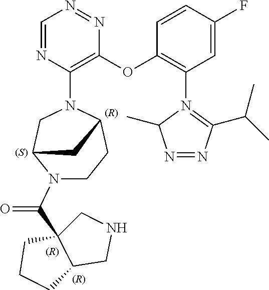

Yellow solid (14 mg, 18% over 2 steps).

¹H NMR (400 MHz, DMSO-d₆, 293.9 K) δ 8.54-8.39 (m, 1H), 7.69-7.42 (m, 3H), 5.26-4.35 (m, 3H), 4.20-3.85 (m, 1H), 3.82-3.70 (m, 3H), 3.68-3.47 (m, 2H), 3.29-3.04 (m, 2H), 2.97-2.63 (m, 4H), 2.45-2.30 (m, 1H), 2.09-1.70 (m, 5H), 1.67-1.50 (m, 3H), 1.48-1.23 (m, 2H), 1.14-0.93 (m, 6H). m/z: ES⁺ [M+H]⁺=562.30.

### Example 3.82 N-cyclopropyl-N-ethyl-5-fluoro-2-((5-((1S,5R)-2-((3aR,6aR)-octahydrocyclopenta[c]pyrrole-3a-carbonyl)-2,6-diazabicyclo[3.2.1]octan-6-yl)-1,2,4-triazin-6-yl)oxy)benzamide

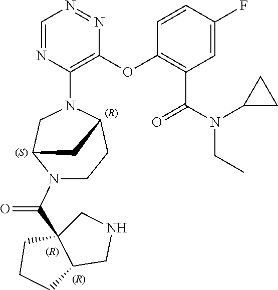

Colourless solid (20 mg, 13% over 2 steps).

¹H NMR (400 MHz, DMSO-d₆, 296.5 K) δ 8.65-8.40 (m, 1H), 8.27 (s, 1H), 7.50-7.45 (m, 2H), 7.44-7.25 (m, 1H), 5.25-4.85 (m, 1H), 4.85-4.40 (m, 1H), 4.39-4.20 (m, 2H), 4.15-3.95 (m, 1H), 3.90-3.70 (m, 2H), 3.65-3.43 (m, 2H), 3.41-3.15 (m, 1H), 3.05-2.95 (m, 2H), 2.90-2.70 (m, 1H), 2.65-2.40 (m, 1H), 2.3-2.15 (m, 1H), 2.12-1.80 (m, 5H), 1.79-1.55 (m, 4H), 1.52-1.30 (m, 2H), 1.10-0.85 (m, 3H), 0.75-0.45 (m, 3H).

m/z: ES⁺ [M+H]⁺=550.30.

### Example 3.83 ((1S,5R)-6-(6-(4-fluoro-2-(4-isopropyl-6-methylpyrimidin-5-yl)phenoxy)-1,2,4-triazin-5-yl)-2,6-diazabicyclo[3.2.1]octan-2-yl)((3aR,6aR)-hexahydrocyclopenta[c]pyrrol-3a(1H)-yl)methanone

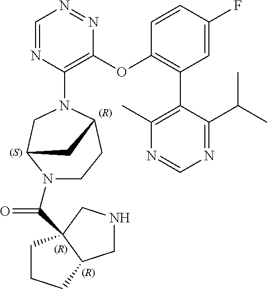

Light brown solid, HCl salt (134 mg, 40% over 2 steps).

¹H NMR (400 MHz, DMSO-d₆, 295.1 K) δ 9.26-8.89 (m, 2H), 7.91-7.68 (m, 1H), 7.68-7.32 (m, 2H), 5.38-4.33 (m, 2H), 4.33-4.12 (m, 1H), 3.92-3.71 (m, 3H), 3.48-3.39 (m, 3H), 3.31-3.22 (m, 1H), 3.19-3.03 (m, 1H), 3.03-2.92 (m, 2H), 2.89-2.77 (m, 1H), 2.33-2.26 (m, 2H), 2.10-1.82 (m, 6H), 1.82-1.71 (m, 2H), 1.63-1.54 (m, 2H), 1.32-0.75 (m, 6H). m/z: ES⁺ [M+H]⁺=573.25.

### Example 3.84 N-ethyl-N-(5-fluoro-2-((5-((1S,5R)-2-((3aR,6aS)-6a-fluorooctahydrocyclopenta[c]pyrrole-3a-carbonyl)-2,6-diazabicyclo[3.2.1]octan-6-yl)-1,2,4-triazin-6-yl)oxy)phenyl)isobutyramide and N-ethyl-N-(5-fluoro-2-((5-((1S,5R)-2-((3aS,6aR)-6a-fluorooctahydrocyclopenta[c]pyrrole-3a-carbonyl)-2,6-diazabicyclo[3.2.1]octan-6-yl)-1,2,4-triazin-6-yl)oxy)phenyl)isobutyramide

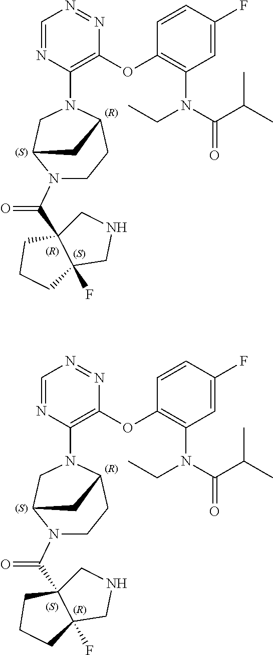

Colourless solid (32 mg, 22% over 2 steps).

¹H NMR (400 MHz, DMSO-d₆, 293.4 K) δ 10.20-10.00 (m, 1H), 9.50-9.05 (m, 1H), 9.00-8.80 (m, 1H), 7.76-7.15 (m, 3H), 5.10-4.90 (m, 2H), 4.76-4.20 (m, 2H), 4.15-3.85 (m, 3H), 3.80-3.40 (m, 3H), 3.21-2.94 (m, 3H), 2.60-2.20 (m, 4H), 2.19-2.00 (m, 2H), 1.95-1.78 (m, 4H), 1.10-0.90 (m, 6H), 0.85-0.65 (m, 4H). m/z: ES⁺ [M+H]⁺=570.30.

### Example 3.85 ((1S,5R)-6-(6-(2-(ethyl(3-isopropyloxetan-3-yl)amino)-4-fluorophenoxy)-1,2,4-triazin-5-yl)-2,6-diazabicyclo[3.2.1]octan-2-yl)((3aR,6aR)-hexahydrocyclopenta[c]pyrrol-3a(1H)-yl)methanone

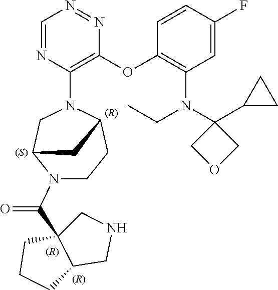

Light brown solid (11 mg, 18% over 2 steps).

¹H NMR (400 MHz, DMSO-d₆, 294.3 K) δ 8.58-8.39 (m, 1H), 7.42-7.28 (m, 1H), 7.25-7.10 (m, 1H), 7.09-6.92 (m, 1H), 5.29-4.99 (m, 1H), 4.85-4.62 (m, 1H), 4.58-4.51 (m, 1H), 4.49-4.38 (m, 1H), 4.37-4.20 (m, 2H), 4.17-3.91 (m, 1H), 3.89-3.59 (m, 1H), 3.58-3.43 (m, 1H), 3.28-2.72 (m, 6H), 2.48-2.38 (m, 3H), 2.21-2.09 (m, 1H), 2.08-1.82 (m, 4H), 1.81-1.58 (m, 4H), 1.48-1.18 (m, 2H), 1.08-0.90 (m, 3H), 0.89-0.62 (m, 6H). m/z: ES⁺ [M+H]⁺=580.25.

### Example 3.86 ((1S,2S,5R)-3-azabicyclo[3.1.0]hexan-2-yl)((1S,5R)-6-(6-(2-(2-cyclopropyl-5-methyl-1H-imidazol-1-yl)-4-fluorophenoxy)-1,2,4-triazin-5-yl)-2,6-diazabicyclo[3.2.1]octan-2-yl)methanone

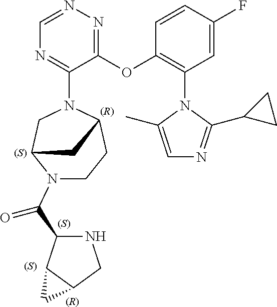

Colourless solid (50 mg, 43% over 2 steps).

¹H NMR (400 MHz, DMSO-d₆, 295.6 K) δ 8.70-8.40 (m, 1H), 7.80-7.60 (m, 2H), 7.59-7.40 (m, 1H), 6.70-6.05 (m, 1H), 5.20-4.80 (m, 1H), 4.76-4.40 (m, 1H), 4.20-3.80 (m, 2H), 3.80-3.70 (m, 1H), 3.70-3.60 (m, 1H), 3.60-3.50 (m, 1H), 3.20-2.90 (m, 1H), 2.80-2.70 (m, 1H), 2.68-2.60 (m, 1H), 2.00-1.80 (m, 5H), 1.70-1.60 (m, 2H), 1.50-1.20 (m, 3H), 0.80-0.70 (m, 3H), 0.68-0.60 (m, 1H), 0.56-0.40 (m, 1H), 0.43-0.30 (m, 1H). m/z: ES⁺ [M+H]⁺=531.25.

### Example 3.87 ((1S,5R)-6-(6-(2-(3-(ethyl(isopropyl)amino)oxetan-3-yl)-4-fluorophenoxy)-1,2,4-triazin-5-yl)-2,6-diazabicyclo[3.2.1]octan-2-yl)((3aR,6aR)-hexahydrocyclopenta[c]pyrrol-3a(1H)-yl)methanone

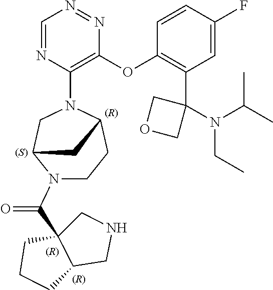

Off-white solid (40 mg, 25% over 2 steps).

¹H NMR (400 MHz, DMSO-d₆, 293.4 K) δ 8.54-8.24 (m, 1H), 7.60-7.50 (m, 1H), 7.50-7.35 (m, 1H), 7.35-7.25 (m, 1H), 5.18-4.73 (m, 6H), 4.18-3.53 (m, 4H), 3.21-3.10 (m, 3H), 3.09-2.81 (m, 4H), 2.75-2.52 (m, 1H), 2.25-1.82 (m, 4H), 1.80-1.52 (m, 5H), 1.45-1.25 (m, 2H), 0.73-0.58 (m, 9H). m/z: ES⁺ [M+H]⁺=580.35.

### Example 3.88 ((1S,5R)-6-(6-(2-(3-(ethyl(isopropyl)amino)oxetan-3-yl)-4-fluorophenoxy)-1,2,4-triazin-5-yl)-2,6-diazabicyclo[3.2.1]octan-2-yl)((S)-2-ethylpyrrolidin-2-yl)methanone

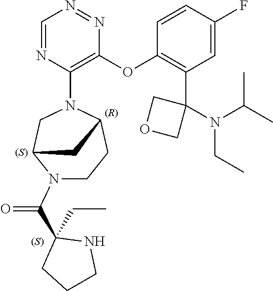

Off-white solid (27 mg, 18% over 2 steps).

¹H NMR (400 MHz, DMSO-d₆, 293.4 K) δ 8.54-8.24 (m, 1H), 7.60-7.50 (m, 1H), 7.50-7.35 (m, 1H), 7.35-7.25 (m, 1H), 5.40-5.20 (m, 1H), 5.18-4.62 (m, 5H), 4.45-3.53 (m, 3H), 3.21-2.81 (m, 5H), 2.75-2.52 (m, 2H), 2.25-1.82 (m, 3H), 1.80-1.52 (m, 7H), 0.73-0.58 (m, 12H). m/z: ES⁺ [M+H]⁺=568.40.

### Examples 4.01 to 4.04

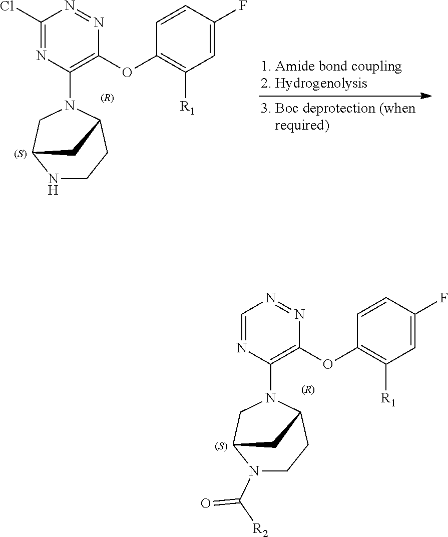

| | | | | Boc deprotection + | |
|---|---|---|---|---|---|
| | Carboxylic acid | Coupling | Step#1 | hydrogenolysis | Step#2 + 3 |
| Ex. | reagent | method | purification | procedure | purification |
| 4.01 | (S)-1-(tert- | A1 | Silica gel, | D1 + B2 | 2. Silica gel, PE/EtOAc 1:1 |
| | butoxycarbonyl)-2- | | PE/EtOAc 1:1 | | 3. Prep-HPLC1; Gradient: 15% |
| | methylpyrrolidine- | | | | B to 38% B in 7 min; Rt = 6.03 |
| | 2-carboxylic acid | | | | min |
| 4.02 | (S)-1-(tert- | A1 | Silica gel, | D1 + B2 | 2. Silica gel, PE/EtOAc 1:1 |
| | butoxycarbonyl)-2- | | PE/EtOAc 1:1 | | 3. Prep-HPLC5; Gradient: 10% |
| | methylpyrrolidine- | | | | B to 33% B in 7 min; Rt = 6.1 min |
| | 2-carboxylic acid | | | | |
| 4.03 | (S)-1-(tert- | A1 | RPFC1, 0% to | D1 + B2 | 2. RPFC1, 0% to 100% MeCN |
| | butoxycarbonyl)-2- | | 100% MeCN | | gradient in 20 min |
| | methylpyrrolidine- | | gradient in 10 | | 3. Prep-HPLC1; Gradient: 16% |
| | 2-carboxylic acid | | min | | B to 39% B in 7 min; Rt = 6.56 |
| | | | | | min |
| 4.04 | (S)-1-(tert- | A1 | RPFC1, 20% | D1 + B1 | 1. RPFC1, 20% to 100% MeCN |
| | butoxycarbonyl)-2- | | to 100% | | gradient in 25 min |
| | methylpyrrolidine- | | MeCN | | 2. Prep-HPLC1; gradient 24% B |
| | 2-carboxylic acid | | gradient in 25 | | to 49% B in 7 min; Rt = 5.72 min |
| | | | min | | |

### Example 4.01: N-methyl-N-(5-fluoro-2-((5-((1S,5R)-2-((S)-2-methylpyrrolidine-2-carbonyl)-2,6-diazabicyclo[3.2.1]octan-6-yl)-1,2,4-triazin-6-yl)oxy)phenyl)isobutyramide

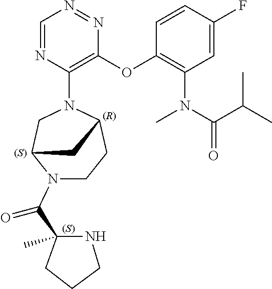

Off-white solid (20 mg, 9% over 3 steps).

¹H NMR (400 MHz, DMSO-d₆) δ 8.60-8.46 (m, 1H), 7.56 (s, 2H), 7.43-7.21 (m, 1H), 5.78-5.12 (m, 1H), 5.12-4.77 (m, 1H), 4.70-4.20 (m, 1H), 4.19-3.90 (m, 1H), 3.88-3.60 (m, 1H), 3.13-2.95 (m, 3H), 2.91-2.71 (m, 2H), 2.68-2.58 (m, 1H), 2.47-2.38 (m, 1H), 2.31-2.15 (m, 1H), 2.09-1.49 (m, 7H), 1.28 (s, 3H), 1.00-0.85 (m, 3H), 0.84-0.60 (m, 3H). (ES, m/z): [M+H]⁺=512.30.

### Example 4.02: N-ethyl-N-(5-fluoro-2-((5-((1S,5R)-2-((S)-2-methylpyrrolidine-2-carbonyl)-2,6-diazabicyclo[3.2.1]octan-6-yl)-1,2,4-triazin-6-yl)oxy)phenyl)isobutyramide

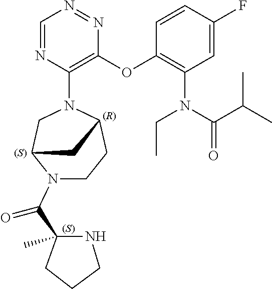

Off-white solid, formic acid (23 mg, 7% over 3 steps).

¹H NMR (400 MHz, DMSO-d₆) δ 8.60-8.47 (m, 1H), 8.28 (s, 1H), 7.66-7.52 (m, 1H), 7.51-7.22 (m, 2H), 5.55-5.15 (m, 1H), 5.08-4.72 (m, 1H), 4.33 (s, 1H), 4.12 (s, 1H), 3.99-3.75 (m, 3H), 3.20-3.00 (m, 2H), 2.91 (s, 2H), 2.78-2.65 (m, 1H), 2.41 (s, 1H), 2.15-1.88 (m, 2H), 1.88-1.5 (m, 6H), 1.33 (s, 3H), 1.10-0.90 (m, 7H), 0.85-0.55 (m, 3H). (ES, m/z): [M+H]⁺ 526.45.

### Example 4.03: N-ethyl-5-fluoro-N-isopropyl-2-((5-((1S,5R)-2-((S)-2-methylpyrrolidine-2-carbonyl)-2,6-diazabicyclo[3.2.1]octan-6-yl)-1,2,4-triazin-6-yl)oxy)benzamide

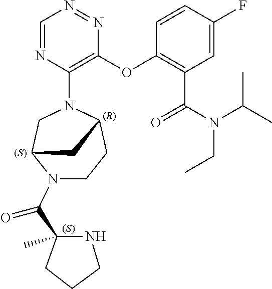

Colourless solid (23 mg, 10% over 3 steps).

¹H NMR (400 MHz, DMSO-d₆, 300 K) δ 8.60-8.45 (m, 1H), 7.63-7.40 (s, 1H), 7.41-7.20 (m, 2H), 5.82-5.25 (m, 1H), 5.15-4.82 (m, 1H), 4.75-4.33 (m, 1H), 4.25-3.93 (m, 1H), 3.85-3.35 (m, 2H), 3.20-2.90 (m, 2H), 2.90-2.75 (m, 2H), 2.75-2.65 (m, 1H), 2.3-2.05 (m, 1H), 2.05-1.81 (m, 3H), 1.80-1.43 (m, 5H), 1.30-1.21 (m, 3H), 1.10-0.85 (m, 7H), 0.80-0.50 (m, 2H). m/z: ES⁺ [M+H]⁺=526.30.

### Example 4.04: ((1S,5R)-6-(6-(2-(2-cyclopropyl-4-methylpyridin-3-yl)-4-fluorophenoxy)-1,2,4-triazin-5-yl)-2,6-diazabicyclo[3.2.1]octan-2-yl)((S)-2-methylpyrrolidin-2-yl)methanone

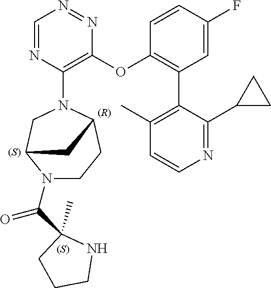

Colourless solid (40 mg, 14% over 3 steps).

¹H NMR (400 MHz, DMSO-d₆, 300 K) δ 8.62-8.40 (m, 1H), 8.25-8.04 (m, 1H), 7.57-7.55 (m, 1H), 7.40-7.05 (m, 2H), 6.98-6.68 (m, 1H), 5.43-4.96 (m, 1H), 4.76-4.64 (m, 1H), 4.52-4.19 (m, 1H), 3.98-3.69 (m, 2H), 3.25-2.62 (m, 4H), 2.21 (s, 1H), 1.97-1.94 (m, 3H), 1.82-1.54 (m, 8H), 1.33-1.12 (m, 3H), 0.93-0.29 (m, 4H). m/z: ES⁺ [M+H]⁺=544.25.

### Examples 5.01 to 5.61

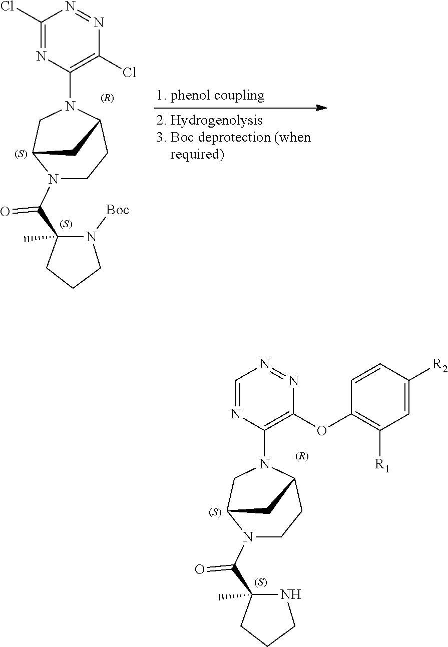

| | | Phenol coupling + | | Boc | |
|---|---|---|---|---|---|
| | | hydrogenolysis | Step#1 + 2 | deprotect. | Step#3 |
| Ex. | Phenol | procedure | purification | procedure | purification |
| 5.01 | N-ethyl-2-hydroxy-N- | C1 + D1 | 1. RPFC1, 10% to 50% | B2 | Prep-HPLC10; |
| | isopropylbenzamide | | MeCN gradient in 10 min | | Gradient: |
| | | | 2. RPFC1, 10% to 50% | | 10% B to 30% B |
| | | | MeCN gradient in 10 min | | in 8 min; Rt = 7.1 |
| | | | | | min |
| 5.02 | 2-(5- | C1 + D1 | 1. RPFC1, 10% to 100% | B2 | Prep-HPLC1; |
| | cyclopropylpyrimidin- | | MeCN gradient in 20 min | | Gradient: |
| | 4-yl)-4-fluorophenol | | 2. RPFC1, 20% to 100% | | 2% B to 20% B |
| | | | MeCN gradient in 25 min | | in 8 min; |
| | | | | | Rt = 7.38 min |
| 5.03 | 2-(4- | C1 + D1 | 1. RPFC1, 10% to 50% | B1 | RPFC1, 10% to |
| | cyclopropylpyrimidin- | | MeCN gradient in 10 min | | 50% MeCN |
| | 5-yl)phenol | | 2. RPFC1, 10% to 50% | | gradient in 10 |
| | | | MeCN gradient in 10 min | | min |
| 5.04 | N,N-dicyclopropyl-5- | C1 + D1 | 1. RPFC1, 10% to 50% | B1 | Prep-HPLC6; |
| | fluoro-2- | | MeCN gradient in 10 min | | Gradient: |
| | hydroxybenzamide | | 2. RPFC1, 10% to 50% | | 2% B to 18% B |
| | | | MeCN gradient in 10 min | | in 8 min; |
| | | | | | Rt = 7.23 min |
| 5.05 | 5-fluoro-2-hydroxy- | C1 + D1 | 1. RPFC1, 10% to 50% | B1 | Prep-HPLC6; |
| | N,N- | | MeCN gradient in 10 min | | Gradient: |
| | dimethylbenzamide | | 2. RPFC1, 10% to 50% | | 1% B to 15% B |
| | | | MeCN gradient in 10 min | | in 7 min; Rt = 5.7 |
| | | | | | min |
| 5.06 | 4-fluoro-2-(2- | C1 + D1 | 1. RPFC3, 0% to 100% | B1 | Prep-HPLC1; |
| | isopropyl-5-methyl- | | MeCN gradient in 30 min | | Gradient: |
| | 1H-imidazol-1- | | 2. RPFC3, 0% to 100% | | 14% B to 39% B |
| | yl)phenol | | MeCN gradient in 30 min | | in 7 min; Rt = 6.8 |
| | | | | | min |
| 5.07 | 4-fluoro-2-(2,5- | C1 + D1 | 1. RPFC3, 10% to 70% | B1 | Prep-HPLC1; |
| | dimethyl-1H- | | MeCN gradient in 30 min | | Gradient: |
| | imidazol-1-yl)phenol | | 2. NPFC1, 10:1 | | 10% B to 35% B |
| | | | DCM/MeOH isocratic | | in 8 min; Rt = 7.9 |
| | | | | | min |
| 5.08 | 5-fluoro-2-hydroxy- | C1 + D1 | 1. RPFC1, 10% to 50% | B2 | Prep-HPLC1; |
| | N-isopropyl-N- | | MeCN gradient in 10 min | | Gradient: 15% B |
| | methylbenzamide | | 2. RPFC1, 10% to 50% | | to 32% B in 7 |
| | | | MeCN gradient in 10 min | | min; Rt = 7.53 |
| | | | | | min |
| 5.09 | 2-(2- | C1 + D1 | 1. RPFC1, 10% to 50% | B2 | Prep-HPLC1; |
| | cyclopropylpyridin-3- | | MeCN gradient in 10 min | | Gradient: |
| | yl)-4-fluorophenol | | 2. RPFC1, 10% to 50% | | 20% B to 45% B |
| | | | MeCN gradient in 10 min | | in 8 min; |
| | | | | | Rt = 7.48 min |
| 5.10 | 2-(4- | C1 + D1 | 1. RPFC1, 40% to 70% | B1 | Prep-HPLC1; |
| | cyclopropylpyridin-3- | | MeCN gradient in 7 min | | Gradient: |
| | yl)-4-fluorophenol | | 2. RPFC1, 30% to 70% | | 16% B to 41% B |
| | | | MeCN gradient in 7 min | | in 7 min; Rt = 6.1 |
| | | | | | min |
| 5.11 | 2- | C1 + D1 | 1. RPFC1, 10% to 100% | B2 | Prep-HPLC1; |
| | (cyclopropylmethoxy)- | | MeCN gradient in 25 min | | Gradient: |
| | 4-fluorophenol | | 2. — | | 21% B to 46% B |
| | | | | | in 7 min; |
| | | | | | Rt = 6.45 min |
| 5.12 | (5-fluoro-2- | C1 + D1 | 1. RPFC1, 10% to 90% | B1 | Prep-HPLC1; |
| | hydroxyphenyl)(pyrrolidin-1- | | MeCN gradient in 20 min | | Gradient: |
| | yl)methanone | | 2. RPFC1, 10% to 90% | | 8% B to 33% B |
| | | | MeCN gradient in 10 min | | in 8 min; Rt = |
| | | | | | 8.22 min |
| 5.13 | (5-fluoro-2- | C1 + D1 | 1. RPFC1, 10% to 90% | B1 | Prep-HPLC6; |
| | hydroxyphenyl)(mor- | | MeCN gradient in 20 min | | Gradient: |
| | pholino)methanone | | 2. RPFC1, 10% to 90% | | 2% B to 18% B |
| | | | MeCN gradient in 20 min | | in 7 min; Rt = |
| | | | | | 5.88 min |
| 5.14 | (3,3-dimethyl-4- | C1 + D1 | 1. RPFC1, 10% to 100% | B1 | Prep-HPLC6; |
| | morpholinyl)(5- | | MeCN gradient in 20 min | | Gradient: |
| | fluoro-2- | | 2. RPFC1, 10% to 100% | | 5% B to 22% B |
| | hydroxyphenyl)methanone | | MeCN gradient in 20 min | | in 7 min; Rt = 6.4 |
| | | | | | min |
| 5.15 | ((1S,4S)-2-oxa-5- | C1 + D1 | 1. RPFC1, 10% to 100% | B2 | Prep-HPLC6; |
| | azabicyclo[2.2.1]heptan- | | MeCN gradient in 20 min | | Gradient: |
| | 5-yl)(5-fluoro-2- | | 2. RPFC1, 10% to 100% | | 2% B to 18% B |
| | hydroxyphenyl)methanone | | MeCN gradient in 20 min | | in 7 min; Rt = 6.2 |
| | | | | | min |
| 5.16 | ((1S,4S)-2-oxa-5- | C1 + D1 | 1. RPFC1, 30% to 50% | B2 | Prep-HPLC1; |
| | azabicyclo[2.2.1]heptan- | | MeCN gradient in 10 min | | Gradient: |
| | 5-yl)(5-fluoro-2- | | 2. RPFC1, 30% to 50% | | 9% B to 27% B |
| | hydroxyphenyl)methanone | | MeCN gradient in 10 min | | in 7 min; |
| | | | | | Rt = 7.43 min |
| 5.17 | ((3S,5S)-3,5- | C1 + D1 | 1. RPFC1, 10% to 50% | B2 | Prep-HPLC6; |
| | dimethylmorpholino) | | MeCN gradient in 10 min | | Gradient: |
| | (5-fluoro-2- | | 2. RPFC1, 10% to 50% | | 25% B to 50% B |
| | hydroxyphenyl)methanone | | MeCN gradient in 10 min | | in 7 min; |
| | | | | | Rt = 6.98 min |
| 5.18 | 2-(2-cyclopropyl-5- | C1 + D1 | 1. RPFC1, 10% to 50% | B1 | RPFC1, 10% to |
| | methyl-1H-imidazol- | | MeCN gradient in 10 min | | 50% MeCN |
| | 1-yl)-4-fluorophenol | | 2. RPFC1, 10% to 50% | | gradient in 10 |
| | | | MeCN gradient in 10 min | | min |
| 5.19 | 2-(3- | C1 + D1 | 1. RPFC4, 40% to 70% | B2 | Prep-HPLC7; |
| | cyclopropylimidazol- | | MeCN gradient in 10 min | | Gradient: 42% B |
| | 4-yl)-4-fluorophenol | | 2. RPFC4, 40% to 70% | | to 67% B in 10 |
| | | | MeCN gradient in 10 min | | min; Rt = 9.53 |
| | | | | | min |
| 5.20 | 6-cyclopropyl-1-(5- | C1 + D1 | 1. RPFC5, 10% to 50% | B1 | Prep-HPLC1; |
| | fluoro-2- | | MeCN gradient in 10 min | | Gradient: 32% B |
| | hydroxyphenyl)pyridin- | | 2. RPFC5, 10% to 50% | | to 48% B in 7 |
| | 2-one | | MeCN gradient in 10 min | | min; Rt = 6.37 |
| | | | | | min |
| 5.21 | 4-fluoro-2-(5- | C1 + D1 | 1. RPFC1, 30% to 70% | B1 | Prep-HPLC1; |
| | methoxy-2- | | MeCN gradient in 10 min | | Gradient: 9% B |
| | methylimidazol-1- | | 2. RPFC1, 30% to 70% | | to 34% B in 8 |
| | yl)phenol | | MeCN gradient in 10 min | | min; Rt = 7.43 |
| | | | | | min |
| 5.22 | 4-fluoro-2-(5- | C1 + D1 | 1. RPFC1, 30% to 70% | B1 | Prep-HPLC1; |
| | isopropoxy-2- | | MeCN gradient in 10 min | | Gradient: 9% B |
| | methylimidazol-1- | | 2. RPFC1, 30% to 70% | | to 34% B in 8 |
| | yl)phenol | | MeCN gradient in 10 min | | min; Rt = 7.40 |
| | | | | | min |
| 5.23 | (S)-1-(tert- | C1 + D1 | 1. RPFC1, 10% to 100% | DB2 | Prep-HPLC6; |
| | butoxycarbonyl)-2- | | MeCN gradient in 25 min | | Gradient: 1% B |
| | methylpyrrolidine-2- | | 2. RPFC1, 10% to 100% | | to 15% B in 6 |
| | carboxylic acid | | MeCN gradient in 20 min | | min; Rt = 7.77 |
| | | | | | min |
| 5.24 | 2-(4,6- | C1 + D1 | 1. RPFC1, 10% to 90% | B1 | Prep-HPLC6; |
| | dicyclopropylpyrimidin- | | MeCN gradient in 20 min | | Gradient: |
| | 5-yl)-4- | | 2. RPFC1, 10% to 90% | | 3% B to 33% B |
| | fluorophenol | | MeCN gradient in 10 min | | in 7 min; Rt = 6.5 |
| | | | | | min |
| 5.25 | 5-fluoro-2-hydroxy- | C1 + D1 | 1. RPFC1, 10% to 100% | B1 | Prep-HPLC1; |
| | N-isopropyl-N-(2,2,2- | | MeCN gradient in 20 min | | Gradient: 19% B |
| | trifluoroethyl)benzamide | | 2. RPFC1, 10% to 90% | | to 44% B in 7 |
| | | | MeCN gradient in 20 min | | min; Rt = 6.35 |
| | | | | | min |
| 5.26 | 2-(4-cyclopropyl-6- | C1 + D1 | 1. RPFC1, 10% to 90% | B1 | Prep-HPLC5; |
| | methylpyrimidin-5- | | MeCN gradient in 25 min | | Gradient: 5% B |
| | yl)-4-fluorophenol | | 2. RPFC1, 10% to 90% | | to 25% B in 7 |
| | | | MeCN gradient in 20 min | | min; Rt = 6.5 min |
| 5.27 | 4-fluoro-2-(5-methyl- | C1 + D1 | 1. FC, PE/EtOAc (1:1) | B2 | Prep-HPLC22; |
| | 2-(trifluoromethyl)- | | 2. RPFC3, 40% to 50% | | Gradient: 9% B |
| | 1H-imidazol-1- | | MeCN gradient in 10 min | | to 39% B in 15 |
| | yl)phenol | | | | min; Rt = 12.7 |
| | | | | | min |
| 5.28 | 2-(2-cyclopropyl-5- | C1 + D1 | 1. RPFC1, 10% to 100% | B2 | Prep-HPLC4; |
| | methoxy-1H- | | MeCN gradient in 20 min | | Gradient: 1% B |
| | imidazol-1-yl)-4- | | 2. RPFC1, 10% to 100% | | to 13% B in 7 |
| | fluorophenol | | MeCN gradient in 20 min | | min; Rt = 6.82 |
| | | | | | min |
| 5.29 | 4-fluoro-2-(2,4,5- | C1 + D1 | 1. RPFC2, 60% to 70% | B2 | Prep-HPLC22; |
| | trimethyl-1H- | | MeCN gradient in 10 min | | Gradient: 0% B |
| | imidazol-1-yl)phenol | | 2. RPFC4, 60% to 90% | | to 20% B in 9 |
| | | | MeCN gradient in 25 min | | min; Rt = 9.07 |
| | | | | | min |
| 5.30 | 2-(2,5-dicyclopropyl- | C1 + D1 | 1. RPFC1, 0% to 100% | B1 | Prep-HPLC1; |
| | 1H-pyrrol-1-yl)-4- | | MeCN gradient in 30 min | | Gradient: 33% B |
| | fluorophenol | | 2. RPFC1, 0% to 100% | | to 53% B in 7 |
| | | | MeCN gradient in 30 min | | min; Rt = 6.57 |
| | | | | | min |
| 5.31 | 2-(4-cyclopropyl-2- | C1 + D1 | 1. RPFC1, 0% to 100% | B2 | Prep-HPLC1; |
| | methylpyridin-3-yl)- | | MeCN gradient in 20 min | | Gradient: 2% B |
| | 4-fluorophenol | | 2. RPFC1, 0% to 100% | | to 15% B in 5 |
| | | | MeCN gradient in 20 min | | min; Rt = 4.2 min |
| 5.32 | 5-cyclopropyl-6-(5- | C1 + D1 | 1. RPFC1, 20% to 80% | B2 | Prep-HPLC23; |
| | fluoro-2- | | MeCN gradient in 20 min | | Gradient: 12% B |
| | hydroxyphenyl)-1- | | 2. RPFC1, 10% to 80% | | to 37% B in 5 |
| | methylpyridin-2(1H)- | | MeCN gradient in 20 min | | min; Rt = 4.56 |
| | one | | | | min |
| 5.33 | 2-(3,5-dicyclopropyl- | C1 + D1 | 1. RPFC1, 10% to 100% | B2 | Prep-HPLC23; |
| | 4H-1,2,4-triazol-4- | | MeCN gradient in 20 min | | Gradient: 9% B |
| | yl)-4-fluorophenol | | 2. Prep-TLC (DCM/MeOH | | to 34% B in 5 |
| | | | 10:1) | | min; Rt = 4.5 min |
| 5.34 | 2-(3,5-diethyl-4H- | C1 + D1 | 1. RPFC1, 10% to 100% | B2 | Prep-HPLC1; |
| | 1,2,4-triazol-4-yl)-4- | | MeCN gradient in 30 min | | Gradient: 13% B |
| | fluorophenol | | 2. RPFC1, 10% to 100% | | to 31% B in 8 |
| | | | MeCN gradient in 20 min | | min; Rt = 7.52 |
| | | | | | min |
| 5.35 | 2-(5-cyclopropyl-2- | C1 + D1 | 1. RPFC1, 0% to 100% | B2 | Prep-HPLC1; |
| | (trifluoromethyl)-1H- | | MeCN gradient in 20 min | | Gradient: 26% B |
| | imidazol-1-yl)-4- | | 2. Prep-TLC (PE/EtOAC | | to 56% B in 8 |
| | fluorophenol | | 1:1) | | min; Rt = 6.68 |
| | | | | | min |
| 5.36 | 2-(5-cyclopropyl-2- | C1 + D1 | 1. RPFC1, 10% to 100% | B1 | Prep-HPLC1; |
| | methyl-1H-imidazol- | | MeCN gradient in 25 min | | Gradient: 17% B |
| | 1-yl)-4-fluorophenol | | 2. RPFC1, 10% to 100% | | to 47% B in 8 |
| | | | MeCN gradient in 25 min | | min; Rt = 7.28 |
| | | | | | min |
| 5.37 | 2-(5-cyclopropyl-3- | C1 + D2 | 1. FC (PE/EtOAc 1:1) | B2 | Prep-HPLC1; |
| | methylisoxazol-4-yl)- | | 2. No purification | | Gradient: 24% B |
| | 4-fluorophenol | | | | to 54% B in 8 |
| | | | | | min; Rt = 6.53 |
| | | | | | min |
| 5.38 | 4-fluoro-2-(1- | C1 + D1 | 1. Prep-TLC (PE/EtOAC | B2 | Prep-HPLC16; |
| | isopropyl-4-methyl- | | 2:1) | | Gradient: 2% B |
| | 1H-1,2,3-triazol-5- | | 2. Prep-TLC (DCM/MeOH | | to 25% B in 7 |
| | yl)phenol | | 20:1) | | min; Rt = 6.12 |
| | | | | | min |
| 5.39 | 2-(3-cyclopropyl-5- | C1 + D1 | 1. RPFC1, 10% to 100% | B2 | Prep-HPLC1; |
| | methyl-4H-1,2,4- | | MeCN gradient in 20 min | | Gradient: 8% B |
| | triazol-4-yl)-4- | | 2. Prep-TLC (DCM/MeOH | | to 38% B in 8 |
| | fluorophenol | | 10:1) | | min; Rt = 7.05 |
| | | | | | min |
| 5.40 | 4-fluoro-2-(1- | C1 + D1 | 1. RPFC1, 10% to 100% | B2 | Trituration with |
| | isopropyl-4-methyl- | | MeCN gradient in 20 min | | EtOAc |
| | 1H-pyrazol-5- | | 2. RPFC1, 10% to 100% | | |
| | yl)phenol | | MeCN gradient in 20 min | | |
| 5.41 | 2-(2-cyclopropyl-5- | C1 + D1 | 1. RPFC1, 10% to 90% | B1 | Prep-HPLC17; |
| | isopropoxy-1H- | | gradient in 20 min | | Gradient 20% B |
| | imidazol-1-yl)-4- | | 2. RPFC1, 10% to 80% | | to 45% B in 10 |
| | fluorophenol | | gradient in 25 min | | min; Rt = 9.97 |
| | | | | | min |
| 5.42 | 4-fluoro-2-(5-(1- | C1 + D1 | 1. RPFC1, 0% to 100% | B1 | Prep-HPLC1; |
| | fluorocyclopropyl)-2- | | MeCN gradient in 15 min | | Gradient 16% B |
| | methyl-1H-imidazol- | | 2. RPFC1, 40% to 80% | | to 41% B in 7 |
| | 1-yl)phenol | | MeCN gradient in 10 min | | min; Rt = 6.87 |
| | | | | | min |
| 5.43 | 2-(4-cyclopropyl-1- | C1 + D1 | 1. RPFC1, 10% to 90% | B2 | RPFC1; |
| | methyl-1H-pyrazol-5- | | MeCN gradient in 30 min | | Gradient 10% B |
| | yl)-4-fluorophenol | | 2. Prep-TLC (DCM/MeOH | | to 90% B in 10 |
| | | | 10:1) | | min; Rt = 9.31 |
| | | | | | min |
| 5.44 | 4-fluoro-2-(4- | C1 + D1 | 1. RPFC1, 10% to 90% | B2 | Prep-HPLC1; |
| | isopropyl-1-methyl- | | MeCN gradient in 30 min | | Gradient 21% B |
| | 1H-pyrazol-5- | | 2. Prep-TLC | | to 51% B in 8 |
| | yl)phenol | | (DCM/MeOH = 10/1) | | min; Rt = 7.68 |
| | | | | | min |
| 5.45 | 4-fluoro-2-(5- | C1 + D1 | 1. RPFC1, 30% to 70% | B2 | Trituration with |
| | isopropyl-3- | | MeCN gradient in 10 min | | EtOAc |
| | methylisoxazol-4- | | 2. RPFC1, 30% to 70% | | |
| | yl)phenol | | MeCN gradient in 10 min | | |
| 5.46 | 4-fluoro-2-(4- | C1 + D1 | 1. RPFC1, 10% to 100% | B2 | Prep-HPLC1; |
| | isopropyl-1-methyl- | | MeCN gradient in 20 min | | Gradient 15% B |
| | 1H-imidazol-5- | | 2. RPFC1, 10% to 100% | | to 45% B in 8 |
| | yl)phenol | | MeCN gradient in 20 min | | min; Rt = 8.85 |
| | | | | | min |
| 5.47 | 2-(3- | C1 + D1 | 1. RPFC3, 0% to 100% | B1 | Prep-HPLC1; |
| | (ethyl(isopropyl)ami- | | MeCN gradient in 15 min | | Gradient 25% B |
| | no)oxetan-3-yl)-4- | | 2. RPFC3, 0% to 100% | | to 45% B in 7 |
| | fluorophenol | | MeCN gradient in 25 min | | min; Rt = 6.62 |
| | | | | | min |
| 5.48 | 4-fluoro-2-(1,3,4- | C1 + D1 | 1. RPFC1, 10% to 100% | B2 | Prep-HPLC6; |
| | trimethyl-1H- | | MeCN gradient in 20 min | | Gradient 5% B |
| | pyrazol-5-yl)phenol | | 2. RPFC1, 10% to 100% | | to 24% B in 7 |
| | | | MeCN gradient in 20 min | | min; Rt = 6.5 |
| | | | | | min |
| 5.49 | 2-(4-cyclopropyl-1,3- | C1 + D1 | 1. RPFC1, 10% to 100% | B2 | Prep-HPLC6; |
| | dimethyl-1H-pyrazol- | | MeCN gradient in 20 min | | Gradient 24% B |
| | 5-yl)-4-fluorophenol | | 2. RPFC1, 10% to 100% | | to 47% B in 10 |
| | | | MeCN gradient in 20 min | | min; Rt = 9.18 |
| | | | | | min |
| 5.50 | 2-(1-cyclopropyl-4- | C1 + D1 | 1. RPFC1, 0% to 100% | B2 | Prep-HPLC6; |
| | methyl-1H-pyrazol-5- | | MeCN gradient in 30 min | | Gradient 5% B |
| | yl)-4-fluorophenol | | 2. RPFC1, 0% to 70% | | to 26% B in 7 |
| | | | MeCN gradient in 20 min | | min; Rt = 6.5 |
| | | | | | min |
| 5.51 | 4-fluoro-2-(5- | C1 + D1 | 1. RPFC1, 10% to 100% | B2 | Prep-HPLC1; |
| | isopropyl-1,3- | | MeCN gradient in 20 min | | Gradient 20% B |
| | dimethyl-1H-pyrazol- | | 2. RPFC1, 10% to 100% | | to 50% B in 8 |
| | 4-yl)phenol | | MeCN gradient in 20 min | | min; Rt = 7.42 |
| | | | | | min |
| 5.52 | 4-fluoro-2-(3- | C1 + D1 | 1. RPFC1, 10% to 100% | B2 | Prep-HPLC1; |
| | isopropyl-1,5- | | MeCN gradient in 25 min | | Gradient 20% B |
| | dimethyl-1H-pyrazol- | | 2. RPFC1, 10% to 100% | | to 50% B in 8 |
| | 4-yl)phenol | | MeCN gradient in 25 min | | min; Rt = 7.38 |
| | | | | | min |
| 5.53 | 2-(3,5-diisopropyl-1- | C1 + D1 | 1. RPFC1, 10% to 100% | B2 | Trituration with |
| | (tetrahydro-2H- | | MeCN gradient in 20 min | | EtOAc |
| | pyran-2-yl)-1H- | | 2. RPFC1, 10% to 100% | | |
| | pyrazol-4-yl)-4- | | MeCN gradient in 20 min | | |
| | fluorophenol | | | | |
| 5.54 | 4-fluoro-2-(5-(1- | C1 + D1 | 1. RPFC4, 0% to 100% | B2 | Prep-HPLC4; |
| | hydroxycyclopropyl)- | | MeCN gradient in 15 min | | Gradient 3% B |
| | 2-methyl-1H- | | 2. RPFC1, 40% to 80% | | to 20% B in 7 |
| | imidazol-1-yl)phenol | | MeCN gradient in 10 min | | min; Rt = 6.3 |
| | | | | | min |
| 5.55 | 4-fluoro-2-(2- | C1 + D1 | 1. RPFC1, 40% to 70% | B2 | 1. Prep- |
| | isopropyl-4,5- | | MeCN gradient in 20 min | | HPLC16; |
| | dimethyl-1H- | | 2. RPFC1, 40% to 70% | | Gradient 2% B |
| | imidazol-1-yl)phenol | | MeCN gradient in 20 min | | to 12% B in 6.5 |
| | | | | | min; Rt = 9.18 |
| | | | | | min |
| | | | | | 2. CHIRALPAKIK3, |
| | | | | | isocratic |
| | | | | | Hex(0.2% |
| | | | | | IPAmine): |
| | | | | | (IPA:DCM = 1:1) = |
| | | | | | 70:30; Rt = 28.25 |
| | | | | | min |
| 5.56 | 4-fluoro-2-(2- | C1 + D1 | 1. RPFC1, 40% to 70% | B2 | 1. Prep- |
| | isopropyl-4,5- | | MeCN gradient in 20 min | | HPLC16; |
| | dimethyl-1H- | | 2. RPFC1, 40% to 70% | | Gradient 2% B |
| | imidazol-1-yl)phenol | | MeCN gradient in 20 min | | to 12% B in 6.5 |
| | | | | | min; Rt = 9.18 |
| | | | | | min |
| | | | | | 2. |
| | | | | | CHIRALPAK3IK, |
| | | | | | isocratic |
| | | | | | Hex(0.2% |
| | | | | | IPAmine): |
| | | | | | (IPA:DCM = 1:1) = |
| | | | | | 70:30; Rt = 33.48 |
| | | | | | min |
| 5.57 | 4-fluoro-2-(5- | C1 + D1 | 1. RPFC1, 10% to 100% | B2 | Prep-HPLC6; |
| | isopropyl-2-methyl- | | MeCN gradient in 15 min | | Gradient 1% B |
| | 1H-imidazol-1- | | 2. RPFC1, 10% to 100% | | to 15% B in 7 |
| | yl)phenol | | MeCN gradient in 20 min | | min; Rt = 4.92 |
| | | | | | min |
| 5.58 | 6-(5-fluoro-2- | C1 + D1 | 1. RPFC1, 10% to 100% | B2 | 1. Prep-HPLC6; |
| | hydroxyphenyl)-1- | | MeCN gradient in 20 min | | Gradient 4% B |
| | isopropyl-5- | | 2. RPFC1, 10% to 100% | | to 28% B in 7 |
| | methylpyridin-2(1H)- | | MeCN gradient in 20 min | | min; Rt = 6.47 |
| | one | | | | min |
| | | | | | 2. CHIRALPAKIK |
| | | | | | isocratic |
| | | | | | Hex(0.2% |
| | | | | | IPAmine): |
| | | | | | (EtOH:DCM = 1:1) = |
| | | | | | 70:30; |
| | | | | | Rt = 6.09 min |
| 5.59 | 6-(5-fluoro-2- | C1 + D1 | 1. RPFC1, 10% to 100% | B2 | 1. Prep-HPLC6; |
| | hydroxyphenyl)-1- | | MeCN gradient in 20 min | | Gradient 4% B |
| | isopropyl-5- | | 2. RPFC1, 10% to 100% | | to 28% B in 7 |
| | methylpyridin-2(1H)- | | MeCN gradient in 20 min | | min; Rt = 6.47 |
| | one | | | | min |
| | | | | | 2. CHIRALPAKIK |
| | | | | | isocratic |
| | | | | | Hex(0.2% |
| | | | | | IPAmine): |
| | | | | | (EtOH:DCM = 1:1) = |
| | | | | | 70:30; |
| | | | | | Rt = 10.79 min |
| 5.60 | 4-fluoro-2-(2- | C1 + D1 | 1. RPFC1, 40% to 70% | B2 | 1. Prep- |
| | isopropyl-4,5- | | MeCN gradient in 20 min | | HPLC25; |
| | dimethyl-1H- | | 2. RPFC1, 40% to 70% | | Gradient 2% B |
| | imidazol-1-yl)phenol | | MeCN gradient in 10 min | | to 15% B in 7 |
| | | | | | min; Rt = 6.1 |
| | | | | | min. |
| | | | | | 2. CHIRALPAKIK |
| | | | | | isocratic |
| | | | | | Hex(0.2% |
| | | | | | IPAmine): |
| | | | | | (IPA:DCM = 1:1) = |
| | | | | | 60:40; Rt = 8.02 min |
| 5.61 | 4-fluoro-2-(2- | C1 + D1 | 1. RPFC1, 40% to 70% | B2 | 1. Prep- |
| | isopropyl-4,5- | | MeCN gradient in 20 min | | HPLC25; |
| | dimethyl-1H- | | 2. RPFC1, 40% to 70% | | Gradient 2% B |
| | imidazol-1-yl)phenol | | MeCN gradient in 10 min | | to 15% B in 7 |
| | | | | | min; Rt = 6.1 |
| | | | | | min. |
| | | | | | 2. CHIRALPAKIK |
| | | | | | isocratic |
| | | | | | Hex(0.2% |
| | | | | | IPAmine): |
| | | | | | (IPA:DCM = 1:1) = |
| | | | | | 60:40; Rt = 11.25 |
| | | | | | min |
| 5.62 | 1-(5-fluoro-2- | C1 + D1 | 1. RPFC1, 50% to 70% | B1 | Prep-HPLC1; |
| | hydroxyphenyl)-6- | | MeCN gradient in 10 min | | Gradient 29% B |
| | isopropylpyridin- | | 2. RPFC1, 60% to 70% | | to 50% B in 7 |
| | 2(1H)-one | | MeCN gradient in 10 min | | min; Rt = 6.38 |
| | | | | | min. |
| 5.63 | 4-fluoro-2-(3- | C1 + D1 | 1. RPFC1, 30% to 80% | B2 | Prep-HPLC6; |
| | isopropyl-5- | | MeCN gradient in 20 min | | Gradient 7% B |
| | methylisoxazol-4- | | 2. RPFC1, 30% to 80% | | to 31% B in 7 |
| | yl)phenol | | MeCN gradient in 10 min | | min; Rt = 6.9 |
| | | | | | min. |
| 5.64 | 2-(1,4-diisopropyl- | C1 + D1 | 1. RPFC1, 10% to 100% | B2 | Prep-HPLC1; |
| | 1H-imidazol-5-yl)-4- | | MeCN gradient in 20 min | | Gradient 20% B |
| | fluorophenol | | 2. RPFC1, 10% to 100% | | to 41% B in 7 |
| | | | MeCN gradient in 20 min | | min; Rt = 6.78 |
| | | | | | min. |
| 5.65 | 2-(2-ethyl-5- | C1 + D1 | 1. RPFC1, 10% to 100% | B2 | Prep-HPLC6; |
| | isopropyl-1H- | | MeCN gradient in 20 min | | Gradient 2% B |
| | imidazol-1-yl)-4- | | 2. RPFC1, 10% to 100% | | to 16% B in 7 |
| | fluorophenol | | MeCN gradient in 20 min | | min; Rt = 6.1 |
| | | | | | min. |
| 5.66 | 4-fluoro-2-(4- | C1 + D2 | 1. Silica gel column | B2 | Precipitation |
| | isopropyl-1-methyl- | | chromatography, | | from EtOAc. |
| | 1H-1,2,3-triazol-5- | | DCM/MeOH (7:1) | | |
| | yl)phenol | | 2. RPFC1, 60% to 70% | | |
| | | | MeCN gradient in 10 min | | |
| 5.67 | 4-fluoro-2-(4- | C1 + D1 | 1. RPFC1, 10% to 100% | B2 | Prep-HPLC1; |
| | isopropyl-1,3- | | MeCN gradient in 20 min | | Gradient 23% B |
| | dimethyl-1H-pyrazol- | | 2. RPFC1, 10% to 100% | | to 43% B in 7 |
| | 5-yl)phenol | | MeCN gradient in 20 min | | min; Rt = 6.67 |
| | | | | | min. |
| 5.68 | 4-fluoro-2-(1- | C1 + D2 | 1. RPFC1, 10% to 100% | B2 | Prep-HPLC6; |
| | isopropyl-3,4- | | MeCN gradient in 25 min | | Gradient 5% B |
| | dimethyl-1H-pyrazol- | | 2. — | | to 29% B in 7 |
| | 5-yl)phenol | | | | min; Rt = 6.7 |
| | | | | | min. |
| 5.69 | 4-fluoro-2-(3- | C1 + D1 | 1. RPFC1, 10% to 50% | B2 | Prep-HPLC1; |
| | (isopropyl(methyl)ami- | | MeCN gradient in 10 min | | Gradient 32% B |
| | no)oxetan-3- | | 2. RPFC1, 10% to 100% | | to 62% B in 8 |
| | yl)phenol | | MeCN gradient in 10 min | | min; Rt = 7.58 |
| | | | | | min. |
| 5.70 | N-ethyl-N-(2-(5- | C1 + D1 | 1. RPFC1, 10% to 100% | B2 | Prep-HPLC6; |
| | fluoro-2- | | MeCN gradient in 20 min | | Gradient 3% B |
| | hydroxyphenyl)propan- | | 2. RPFC1, 10% to 100% | | to 22% B in 7 |
| | 2-yl)acetamide | | MeCN gradient in 20 min | | min; Rt = 6.97 |
| | | | | | min. |
| 5.71 | 2-(3- | C1 + D1 | 1. RPFC1, 10% to 100% | B1 | Prep-HPLC11; |
| | (dimethylamino)oxetan- | | MeCN gradient in 10 min | | Gradient 2% B |
| | 3-yl)-4- | | 2. RPFC1, 10% to 100% | | to 15% B in 7 |
| | fluorophenol | | MeCN gradient in 10 min | | min; Rt = 6.1 |
| | | | | | min. |
| 5.72 | 4-fluoro-2-(5- | C1 + D1 | 1. RPFC1, 10% to 100% | B2 | Prep-HPLC24; |
| | isopropyl-4-methyl- | | MeCN gradient in 25 min | | Gradient 2% B |
| | 1H-imidazol-1- | | 2. RPFC1, 10% to 100% | | to 10% B in 7 |
| | yl)phenol | | MeCN gradient in 25 min | | min; Rt = 6.0 |
| | | | | | min. |
| 5.73 | 4-fluoro-2-(2- | C1 + D1 | 1. RPFC1, 10% to 100% | B2 | Trituration with |
| | isopropyl-4-methyl- | | MeCN gradient in 20 min | | EtOAc. |
| | 1H-imidazol-1- | | 2. Prep TLC, DCM/MeOH | | |
| | yl)phenol | | 10:1 | | |
| 5.74 | 4-(5-fluoro-2- | C1 + D2 | 1. RPFC1, 10% to 500% | B2 | Prep-HPLC6; |
| | hydroxyphenyl)-3- | | MeCN gradient in 10 min | | Gradient 4% B |
| | isopropyl-1,5- | | 2. — | | to 30% B in 7 |
| | dimethylpyridin- | | | | min; Rt = 7.3 |
| | 2(1H)-one | | | | min. |
| 5.75 | 4-(5-fluoro-2- | C1 + D1 | 1. RPFC1, 10% to 50% | B2 | 1. Prep-HPLC6; |
| | hydroxyphenyl)-5- | | MeCN gradient in 10 min | | Gradient 5% B |
| | isopropyl-1,3- | | 2. RPFC1, 10% to 100% | | to 28% B in 7 |
| | dimethylpyridin- | | MeCN gradient in 10 min | | min; Rt = 6.37 |
| | 2(1H)-one | | | | min. |
| | | | | | 2. |
| | | | | | CHIRALPAKIK3 |
| | | | | | Isocratic A: |
| | | | | | Hex(0.2% |
| | | | | | IPAmine), B: |
| | | | | | iPA:DCM = 1:1; |
| | | | | | 60% B; Rt = |
| | | | | | 12.38 min. |
| 5.76 | 4-(5-fluoro-2- | C1 + D1 | 1. RPFC1, 10% to 50% | B2 | 1. Prep-HPLC6; |
| | hydroxyphenyl)-5- | | MeCN gradient in 10 min | | Gradient 5% B |
| | isopropyl-1,3- | | 2. RPFC1, 10% to 100% | | to 28% B in 7 |
| | dimethylpyridin- | | MeCN gradient in 10 min | | min; Rt = 6.37 |
| | 2(1H)-one | | | | min. |
| | | | | | 2. |
| | | | | | CHIRALPAKIK3 |
| | | | | | Isocratic A: |
| | | | | | Hex(0.2% |
| | | | | | IPAmine), B: |
| | | | | | iPA:DCM = 1:1; |
| | | | | | 60% B; Rt = |
| | | | | | 16.72 min. |
| 5.77 | 6-(5-fluoro-2- | C1 + D1 | 1. RPFC1, 10% to 100% | B2 | Prep-HPLC1; |
| | hydroxyphenyl)-5- | | MeCN gradient in 25 min | | Gradient 11% B |
| | isopropyl-1- | | 2. RPFC1, 10% to 100% | | to 36% B in 7 |
| | methylpyrimidin- | | MeCN gradient in 25 min | | min; Rt = 6.88 |
| | 2(1H)-one | | | | min. |
| 5.78 | 5-(5-fluoro-2- | C1 + D1 | 1. RPFC1, 20% to 50% | B2 | Prep-HPLC6; |
| | hydroxyphenyl)-6- | | MeCN gradient in 10 min | | Gradient 7% B |
| | isopropyl-1- | | 2. RPFC1, 30% to 50% | | to 37% B in 7 |
| | methylpyrazin-2(1H)- | | MeCN gradient in 10 min | | min; Rt = 6.1 |
| | one | | | | min. |
| 5.79 | 4-fluoro-2-(5- | C1 + D1 | 1. RPFC1, 10% to 100% | B2 | 1. Prep-HPLC1; |
| | isopropyl-2,4- | | MeCN gradient in 10 min | | Gradient 23% B |
| | dimethyl-1H- | | 2. Prep TLC, DCM/MeOH | | to 41% B in 8 |
| | imidazol-1-yl)phenol | | 10:1 | | min; Rt = 7.17 |
| | | | | | min. |
| | | | | | 2. |
| | | | | | CHIRALPAKIK3 |
| | | | | | Isocratic A: |
| | | | | | Hex(0.2% |
| | | | | | IPAmine), B: |
| | | | | | iPA:DCM = 1:1; |
| | | | | | 40% B; Rt = |
| | | | | | 11.25 min. |
| 5.80 | 4-fluoro-2-(5- | C1 + D1 | 1. RPFC1, 10% to 100% | B2 | 1. Prep-HPLC1; |
| | isopropyl-2,4- | | MeCN gradient in 10 min | | Gradient 23% B |
| | dimethyl-1H- | | 2. Prep TLC, DCM/MeOH | | to 41% B in 8 |
| | imidazol-1-yl)phenol | | 10:1 | | min; Rt = 7.17 |
| | | | | | min. |
| | | | | | 2. |
| | | | | | CHIRALPAKIK3 |
| | | | | | Isocratic A: |
| | | | | | Hex(0.2% |
| | | | | | IPAmine), B: |
| | | | | | iPA:DCM = 1:1; |
| | | | | | 40% B; Rt = |
| | | | | | 14.33 min. |
| 5.81 | 3,4-difluoro-2-(2- | C1 + D1 | 1. RPFC1, 75% to 80% | B2 | 1. Prep-HPLC6; |
| | isopropyl-4,5- | | MeCN gradient in 10 min | | Gradient 2% B |
| | dimethyl-1H- | | 2. RPFC1, 70% to 80% | | to 15% B in 8 |
| | imidazol-1-yl)phenol | | MeCN gradient in 10 min | | min; Rt = 6.5 |
| | | | | | min. |
| | | | | | 2. |
| | | | | | CHIRALART2 |
| | | | | | Isocratic A: |
| | | | | | Hex(0.2% |
| | | | | | IPAmine), B: |
| | | | | | iPA:DCM = 1:1; |
| | | | | | 50% B; Rt = |
| | | | | | 20.22 min. |
| 5.82 | 3,4-difluoro-2-(2- | C1 + D1 | 1. RPFC1, 75% to 80% | B2 | 1. Prep-HPLC6; |
| | isopropyl-4,5- | | MeCN gradient in 10 min | | Gradient 2% B |
| | dimethyl-1H- | | 2. RPFC1, 70% to 80% | | to 15% B in 8 |
| | imidazol-1-yl)phenol | | MeCN gradient in 10 min | | min; Rt = 6.5 |
| | | | | | min. |
| | | | | | 2. |
| | | | | | CHIRALART2 |
| | | | | | Isocratic A: |
| | | | | | Hex(0.2% |
| | | | | | IPAmine), B: |
| | | | | | iPA:DCM = 1:1; |
| | | | | | 50% B; Rt = |
| | | | | | 28.37 min. |
| 5.83 | 2-(2,4-diisopropyl- | C1 + D1 | 1. Silica gel column | B2 | Prep-HPLC6; |
| | 1H-imidazol-1-yl)-4- | | chromatography, | | Gradient 2% B |
| | fluorophenol | | DCM/MeOH (10:1) | | to 13% B in 7 |
| | | | 2. RPFC1, 70% to 80% | | min; Rt = 6.1 |
| | | | MeCN gradient in 10 min | | min. |
| 5.84 | rac-2-(1- | C1 + D2 | 1. RPFC1, 10% to 100% | B2 | Trituration with |
| | (dimethylamino)- | | MeCN gradient in 10 min | | EtOAc. |
| | 2,2,2-trifluoroethyl)- | | 2. Prep TLC, DCM/MeOH | | |
| | 4-fluorophenol | | 10:1 | | |
| 5.85 | 2-(2-(tert-butyl)-4,5- | C1 + D2 | 1. RPFC1, 10% to 100% | B2 | Prep-HPLC6; |
| | dimethyl-1H- | | MeCN gradient in 10 min | | Gradient 2% B |
| | imidazol-1-yl)-4- | | 2. RPFC1, 10% to 50% | | to 12% B in 7 |
| | fluorophenol | | MeCN gradient in 10 min | | min; Rt = 6.9 |
| | | | | | min. |
| 5.86 | 2-(4-ethyl-2- | C1 + D1 | 1. RPFC1, 60% to 80% | B2 | Prep-HPLC6; |
| | isopropyl-5-methyl- | | MeCN gradient in 10 min | | Gradient 2% B |
| | 1H-imidazol-1-yl)-4- | | 2. RPFC1, 60% to 70% | | to 12% B in 7 |
| | fluorophenol | | MeCN gradient in 10 min | | min; Rt = 6.63 |
| | | | | | min. |
| 5.87 | 2-(4-ethyl-5- | C1 + D1 | 1. RPFC4, 30% to 90% | B2 | 1. Prep-HPLC6; |
| | isopropyl-2-methyl- | | MeCN gradient in 10 min | | Gradient 2% B |
| | 1H-imidazol-1-yl)-4- | | 2. RPFC1, 40% to 90% | | to 18% B in 7 |
| | fluorophenol | | MeCN gradient in 10 min | | min; Rt = 6.9 |
| | | | | | min. |
| | | | | | 2. |
| | | | | | CHIRALPAKIK3 |
| | | | | | Isocratic A: |
| | | | | | Hex(0.2% |
| | | | | | IPAmine), B: |
| | | | | | iPA:DCM = 1:1; |
| | | | | | 30% B; Rt = |
| | | | | | 24.46 min. |
| 5.88 | 2-(4-ethyl-5- | C1 + D1 | 1. RPFC4, 30% to 90% | B2 | 1. Prep-HPLC6; |
| | isopropyl-2-methyl- | | MeCN gradient in 10 min | | Gradient 2% B |
| | 1H-imidazol-1-yl)-4- | | 2. RPFC1, 40% to 90% | | to 18% B in 7 |
| | fluorophenol | | MeCN gradient in 10 min | | min; Rt = 6.9 |
| | | | | | min. |
| | | | | | 2. |
| | | | | | CHIRALPAKIK3 |
| | | | | | Isocratic A: |
| | | | | | Hex(0.2% |
| | | | | | IPAmine), B: |
| | | | | | iPA:DCM = 1:1; |
| | | | | | 30% B; Rt = |
| | | | | | 28.66 min. |
| 5.89 | 4-fluoro-2-(2- | C1 + D1 | 1. RPFC1, 10% to 50% | B2 | Prep-HPLC6; |
| | isopropyl-5,6- | | MeCN gradient in 10 min | | Gradient 2% B |
| | dihydrocyclopenta[d] | | 2. RPFC1, 10% to 50% | | to 17% B in 7 |
| | imidazol-1(4H)- | | MeCN gradient in 10 min | | min; Rt = 5.8 |
| | yl)phenol | | | | min. |
| 5.90 | 4-fluoro-2-(2-methyl- | C1 + D1 | 1. RPFC1, 10% to 100% | B2 | Trituration with |
| | 6,7-dihydro-5H- | | MeCN gradient in 20 min | | EtOAc. |
| | pyrrolo[1,2- | | 2. RPFC1, 10% to 100% | | |
| | a]imidazol-3- | | MeCN gradient in 25 min | | |
| | yl)phenol | | | | |
| 5.91 | 4-fluoro-2-(2- | C1 + D1 | 1. RPFC1, 10% to 100% | B2 | Prep-HPLC1; |
| | isopropyl-6,7- | | MeCN gradient in 20 min | | Gradient 17% B |
| | dihydro-5H- | | 2. RPFC1, 10% to 100% | | to 47% B in 8 |
| | pyrrolo[1,2- | | MeCN gradient in 25 min | | min; Rt = 7.4 |
| | a]imidazol-3- | | | | min. |
| | yl)phenol | | | | |
| 5.92 | 2-(5-ethyl-2- | C1 + D1 | 1. RPFC1, 10% to 100% | B2 | 1. Prep-HPLC6; |
| | isopropyl-4-methyl- | | MeCN gradient in 20 min | | Gradient 2% B |
| | 1H-imidazol-1-yl)-4- | | 2. RPFC1, 10% to 100% | | to 13% B in 7 |
| | fluorophenol | | MeCN gradient in 20 min | | min; Rt = 6.8 |
| | | | | | min. |
| | | | | | 2. |
| | | | | | CHIRALPAKIK3 |
| | | | | | Isocratic A: |
| | | | | | Hex(0.2% |
| | | | | | IPAmine), B: |
| | | | | | iPA:DCM = 1:1; |
| | | | | | 30% B; Rt = |
| | | | | | 19.90 min. |
| 5.93 | 2-(5-ethyl-2- | C1 + D1 | 1. RPFC1, 10% to 100% | B2 | 1. Prep-HPLC6; |
| | isopropyl-4-methyl- | | MeCN gradient in 20 min | | Gradient 2% B |
| | 1H-imidazol-1-yl)-4- | | 2. RPFC1, 10% to 100% | | to 13% B in 7 |
| | fluorophenol | | MeCN gradient in 20 min | | min; Rt = 6.8 |
| | | | | | min. |
| | | | | | 2. |
| | | | | | CHIRALPAKIK3 |
| | | | | | Isocratic A: |
| | | | | | Hex(0.2% |
| | | | | | IPAmine), B: |
| | | | | | iPA:DCM = 1:1; |
| | | | | | 30% B; Rt = |
| | | | | | 29.29 min. |
| 5.94 | 2-(2,5-diisopropyl-4- | C1 + D1 | 1. RPFC1, 10% to 100% | B2 | 1. Prep-HPLC6; |
| | methyl-1H-imidazol- | | MeCN gradient in 30 min | | Gradient 2% B |
| | 1-yl)-4-fluorophenol | | 2. RPFC1, 10% to 100% | | to 15% B in 7 |
| | | | MeCN gradient in 30 min | | min; Rt = 6.9 |
| | | | | | min. |
| | | | | | 2. |
| | | | | | CHIRALPAKIK3 |
| | | | | | Isocratic A: |
| | | | | | MtBE(0.5% 2M |
| | | | | | NH₃—MeOH), B: |
| | | | | | iPA:DCM = 1:1; |
| | | | | | 25% B; Rt = |
| | | | | | 13.0 min. |
| 5.95 | 2-(2,5-diisopropyl-4- | C1 + D1 | 1. RPFC1, 10% to 100% | B2 | 1. Prep-HPLC6; |
| | methyl-1H-imidazol- | | MeCN gradient in 30 min | | Gradient 2% B |
| | 1-yl)-4-fluorophenol | | 2. RPFC1, 10% to 100% | | to 15% B in 7 |
| | | | MeCN gradient in 30 min | | min; Rt = 6.9 |
| | | | | | min. |
| | | | | | 2. |
| | | | | | CHIRALPAKIK3 |
| | | | | | Isocratic A: |
| | | | | | MtBE(0.5% 2M |
| | | | | | NH₃—MeOH), B: |
| | | | | | iPA:DCM = 1:1; |
| | | | | | 25% B; Rt = |
| | | | | | 15.64 min. |
| 5.96 | 5-(5-fluoro-2- | C1 + D1 | 1. RPFC1, 10% to 100% | B2 | 1. Prep-HPLC6; |
| | hydroxyphenyl)-4- | | MeCN gradient in 20 min | | Gradient 5% B |
| | isopropyl-1,6- | | 2. RPFC1, 10% to 100% | | to 28% B in 7 |
| | dimethylpyridin- | | MeCN gradient in 20 min | | min; Rt = 6.5 |
| | 2(1H)-one | | | | min. |
| | | | | | 2. |
| | | | | | CHIRALPAKIH |
| | | | | | Isocratic A: |
| | | | | | Hex(0.2% |
| | | | | | IPAmine), B: |
| | | | | | EtOH:DCM = 1:1; |
| | | | | | 35% B; Rt = |
| | | | | | 4.28 min. |
| 5.97 | 5-(5-fluoro-2- | C1 + D1 | 1. RPFC1, 10% to 100% | B2 | 1. Prep-HPLC6; |
| | hydroxyphenyl)-4- | | MeCN gradient in 20 min | | Gradient 5% B |
| | isopropyl-1,6- | | 2. RPFC1, 10% to 100% | | to 28% B in 7 |
| | dimethylpyridin- | | MeCN gradient in 20 min | | min; Rt = 6.5 |
| | 2(1H)-one | | | | min. |
| | | | | | 2. |
| | | | | | CHIRALPAKIH |
| | | | | | Isocratic A: |
| | | | | | Hex(0.2% |
| | | | | | IPAmine), B: |
| | | | | | EtOH:DCM = 1:1; |
| | | | | | 35% B; Rt = |
| | | | | | 8.77 min. |
| 5.98 | 1-(5-fluoro-2- | C1 + D1 | 1. Silica gel column | B2 | Prep-HPLC6; |
| | hydroxyphenyl)-2,6- | | chromatography, PE/EtOAc | | Gradient 1% B |
| | dimethylpyridin- | | (1:1) | | to 15% B in 8 |
| | 4(1H)-one | | 2. RPFC1, 10% to 50% | | min; Rt = 7.8 |
| | | | MeCN gradient in 10 min | | min. |
| 5.99 | 1-(5-fluoro-2- | C1 + D1 | 1. RPFC1, 10% to 50% | B2 | Prep-HPLC6; |
| | hydroxyphenyl)-2,6- | | MeCN gradient in 20 min | | Gradient 5% B |
| | diisopropylpyridin- | | 2. — | | to 21% B in 7 |
| | 4(1H)-one | | | | min; Rt = 6.8 |
| | | | | | min. |
| 5.100 | 4-fluoro-2-(4- | C1 + D1 | 1. RPFC1, 10% to 100% | B2 | Trituration with |
| | isopropyl-1,2- | | MeCN gradient in 20 min | | EtOAc. |
| | dimethyl-1H- | | 2. Silica gel column | | |
| | imidazol-5-yl)phenol | | chromatography, | | |
| | | | DCM/MeOH (10:1) | | |
| 5.101 | 4-fluoro-2-(1- | C1 + D1 | 1. RPFC1, 10% to 100% | B2 | Prep-HPLC1; |
| | isopropyl-2,5- | | MeCN gradient in 20 min | | Gradient 15% B |
| | dimethyl-1H- | | 2. RPFC1, 10% to 100% | | to 40% B in 7 |
| | imidazol-4-yl)phenol | | MeCN gradient in 20 min | | min; Rt = 6.08 |
| | | | | | min. |
| 5.102 | 6-(5-fluoro-2- | C1 + D1 | 1. RPFC1, 10% to 100% | B2 | 1. Prep-HPLC1; |
| | hydroxyphenyl)-5- | | MeCN gradient in 25 min | | Gradient 20% B |
| | isopropyl-1,4- | | 2. Silica gel column | | to 42% B in 7 |
| | dimethylpyridin- | | chromatography, | | min; Rt = 6.97 |
| | 2(1H)-one | | DCM/MeOH (10:1) | | min. |
| | | | | | 2. |
| | | | | | CHIRALPAKIF |
| | | | | | Isocratic A: |
| | | | | | Hex(0.2% |
| | | | | | IPAmine), B: |
| | | | | | EtOH:DCM = 1:1; |
| | | | | | 40% B; Rt = |
| | | | | | 13.25 min. |
| 5.103 | 6-(5-fluoro-2- | C1 + D1 | 1. RPFC1, 10% to 100% | B2 | 1. Prep-HPLC1; |
| | hydroxyphenyl)-5- | | MeCN gradient in 25 min | | Gradient 20% B |
| | isopropyl-1,4- | | 2. Silica gel column | | to 42% B in 7 |
| | dimethylpyridin- | | chromatography, | | min; Rt = 6.97 |
| | 2(1H)-one | | DCM/MeOH (10:1) | | min. |
| | | | | | 2. |
| | | | | | CHIRALPAKIF |
| | | | | | Isocratic A: |
| | | | | | Hex(0.2% |
| | | | | | IPAmine), B: |
| | | | | | EtOH:DCM = 1:1; |
| | | | | | 40% B; Rt = |
| | | | | | 17.33 min. |
| 5.104 | 5-(5-fluoro-2- | C1 + D1 | 1. RPFC1, 10% to 100% | B2 | Prep-HPLC9; |
| | hydroxyphenyl)-6- | | MeCN gradient in 35 min | | Gradient 20% B |
| | isopropyl-1- | | 2. RPFC1, 10% to 90% | | to 45% B in 15 |
| | methylpyridin-2(1H)- | | MeCN gradient in 25 min | | min; Rt = 14.5 |
| | one | | | | min. |
| 5.105 | 4-fluoro-2-(5- | C1 + D1 | 1. RPFC1, 10% to 100% | B2 | Precipitation |
| | isopropyl-6- | | MeCN gradient in 25 min | | from EtOAc. |
| | methylpyridazin-4- | | 2. RPFC1, 10% to 100% | | |
| | yl)phenol | | MeCN gradient in 25 min | | |
| 5.106 | 4-fluoro-2-(5- | C1 + D1 | 1. RPFC1, 10% to 50% | B2 | 1. Prep-HPLC2; |
| | isopropyl-3- | | MeCN gradient in 10 min | | Gradient 20% B |
| | methylpyridazin-4- | | 2. RPFC1, 10% to 70% | | to 42% B in 7 |
| | yl)phenol | | MeCN gradient in 10 min | | min; Rt = 6.97 |
| | | | | | min. |
| | | | | | 2. CHIRALART |
| | | | | | Isocratic A: |
| | | | | | MTBE (with |
| | | | | | 0.5% NH₃ (2M |
| | | | | | in MeOH), B: |
| | | | | | IPA:DCM = 1:1; |
| | | | | | 70% B; Rt = |
| | | | | | 11.77 min. |
| 5.107 | 4-fluoro-2-(5- | C1 + D1 | 1. RPFC1, 10% to 50% | B2 | 1. Prep-HPLC2; |
| | isopropyl-3- | | MeCN gradient in 10 min | | Gradient 20% B |
| | methylpyridazin-4- | | 2. RPFC1, 10% to 70% | | to 42% B in 7 |
| | yl)phenol | | MeCN gradient in 10 min | | min; Rt = 6.97 |
| | | | | | min. |
| | | | | | 2. CHIRALART |
| | | | | | Isocratic A: |
| | | | | | MTBE (with |
| | | | | | 0.5% NH₃ (2M |
| | | | | | in MeOH), B: |
| | | | | | IPA:DCM = 1:1; |
| | | | | | 70% B; Rt = |
| | | | | | 18.31 min. |
| 5.108 | 4-fluoro-2-(1- | C1 + D1 | 1. RPFC1, 10% to 100% | B2 | Prep-HPLC2; |
| | isopropyl-2-methyl- | | MeCN gradient in 20 min | | Gradient 41% B |
| | 1H-imidazol-5- | | 2. RPFC1, 10% to 100% | | to 66% B in 10 |
| | yl)phenol | | MeCN gradient in 20 min | | min; Rt = 10.52 |
| | | | | | min. |
| 5.109 | 4-fluoro-2-(1- | C1 + D1 | 1. RPFC1, 10% to 50% | B2 | Prep-HPLC6; |
| | isopropyl-2,4- | | MeCN gradient in 10 min | | Gradient 2% B |
| | dimethyl-1H- | | 2. RPFC1, 10% to 50% | | to 13% B in 10 |
| | imidazol-5-yl)phenol | | MeCN gradient in 10 min | | min; Rt = 5.28 |
| | | | | | min. |
| 5.110 | 2-cyclopropyl-1-(5- | C1 + D1 | 1. RPFC1, 30% to 100% | B2 | Prep-HPLC6; |
| | fluoro-2- | | MeCN gradient in 20 min | | Gradient 6% B |
| | methoxyphenyl)-5- | | 2. RPFC1, 30% to 90% | | to 36% B in 8 |
| | (trifluoromethyl)-1H- | | MeCN gradient in 10 min | | min; Rt = 7.13 |
| | imidazole | | | | min. |
| 5.111 | (S)-4-fluoro-2- | C1 + D1 | 1. RPFC1, 10% to 85% | B2 | Prep-HPLC6; |
| | ((tetrahydrofuran-3- | | MeCN gradient in 30 min | | Gradient 1% B |
| | yl)oxy)phenol | | 2. RPFC1, 10% to 100% | | to 24% B in 8 |
| | | | MeCN gradient in 30 min | | min; Rt = 6.48 |
| | | | | | min. |
| 5.112 | (R)-4-fluoro-2- | C1 + D1 | 1. Prep-TLC PE/EtOAc | B2 | Trituration with |
| | ((tetrahydrofuran-3- | | (3:1) | | EtOAc. |
| | yl)oxy)phenol | | 2. — | | |
| 5.113 | 5-(5-fluoro-2- | C1 + D1 | 1. — | B2 | Prep-HPLC6; |
| | hydroxyphenyl)-6- | | 2. — | | Gradient 4% B |
| | isopropyl-4- | | | | to 25% B in 7 |
| | methylpyridin-2(1H)- | | | | min; Rt = 6.8 |
| | one | | | | min. |
| 5.114 | 4-fluoro-2-(1- | C1 + D1 | 1. RPFC1, 80% to 90% | B2 | Prep-HPLC1; |
| | isopropyl-4,5- | | MeCN gradient in 10 min | | Gradient 16% B |
| | dimethyl-1H- | | 2. RPFC1, 70% to 80% | | to 41% B in 7 |
| | imidazol-2-yl)phenol | | MeCN gradient in 10 min | | min; Rt = 6.33 |
| | | | | | min. |
| 5.115 | 4-fluoro-2-(5- | C1 + D1 | 1. RPFC1, 10% to 100% | B2 | Prep-HPLC6; |
| | isopropyl-1-methyl- | | MeCN gradient in 20 min | | Gradient 1% B |
| | 1H-imidazol-4- | | 2. RPFC1, 10% to 100% | | to 19% B in 8 |
| | yl)phenol | | MeCN gradient in 20 min | | min; Rt = 7.22 |
| | | | | | min. |
| 5.116 | N-ethyl-N-(5-fluoro- | C1 + D1 | 1. RPFC1, 10% to 100% | B2 | Prep-HPLC6; |
| | 2- | | MeCN gradient in 10 min | | Gradient 1% B |
| | hydroxybenzyl)acetamide | | 2. RPFC1, 10% to 100% | | to 25% B in 8 |
| | | | MeCN gradient in 10 min | | min; Rt = 6.03 |
| | | | | | min. |
| 5.117 | N-(5-fluoro-2- | C1 + D1 | 1. RPFC1, 10% to 100% | B2 | Prep-HPLC6; |
| | hydroxybenzyl)-N- | | MeCN gradient in 20 min | | Gradient 3% B |
| | isopropylacetamide | | 2. RPFC1, 10% to 100% | | to 20% B in 7 |
| | | | MeCN gradient in 20 min | | min; Rt = 6.5 |
| | | | | | min. |
| 5.118 | 1-(5-fluoro-2- | C1 + D1 | 1. Silica gel column | B2 | Prep-HPLC1; |
| | hydroxybenzyl)pyrrolidin- | | chromatography, PE/EtOAc | | Gradient 9% B |
| | 2-one | | (1:1) | | to 34% B in 7 |
| | | | 2. Prep-TLC DCM/MeOH | | min; Rt = 6.43 |
| | | | (20:1) | | min. |
| 5.119 | (R)-1-(5-fluoro-2- | C1 + D1 | 1. Prep-TLC PE/EtOAc | B2 | Prep-HPLC1; |
| | hydroxybenzyl)-5- | | (3:1) | | Gradient 11% B |
| | methylpyrrolidin-2- | | 2. RPFC1, 0% to 100% | | to 41% B in 8 |
| | one | | MeCN gradient in 30 min | | min; Rt = 7.3 |
| | | | | | min. |
| 5.120 | 4-fluoro-2-(5- | C1 + D1 | 1. RPFC1, 10% to 100% | B2 | Prep-HPLC6; |
| | isopropyl-1,2- | | MeCN gradient in 20 min | | Gradient 2% B |
| | dimethyl-1H- | | 2. RPFC2, 0% to 80% | | to 12% B in 7 |
| | imidazol-4-yl)phenol | | MeCN gradient in 20 min | | min; Rt = 6.9 |
| | | | | | min. |
| 5.121 | 4-fluoro-2-(1- | C1 + D1 | 1. RPFC1, 10% to 100% | B2 | Prep-HPLC6; |
| | isopropyl-2-methyl- | | MeCN gradient in 10 min | | Gradient 2% B |
| | 1H-imidazol-4- | | 2. RPFC1, 10% to 100% | | to 20% B in 7 |
| | yl)phenol | | MeCN gradient in 20 min | | min; Rt = 6.2 |
| | | | | | min. |

### Example 5.01: N-ethyl-N-isopropyl-2-((5-((1S,5R)-2-((S)-2-methylpyrrolidine-2-carbonyl)-2,6-diazabicyclo[3.2.1]octan-6-yl)-1,2,4-triazin-6-yl)oxy)benzamide

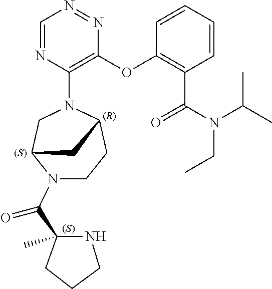

Colourless solid (31 mg, 23% over 3 steps).

¹H NMR (400 MHz, DMSO-d₆,295 K) δ 8.61-8.47 (m, 1H), 8.42-8.28 (m, 1H), 7.61-7.08 (m, 4H), 5.23-5.09 (m, 2H), 4.88-4.68 (m, 1H), 3.94-3.54 (m, 2H), 3.43-3.27 (m, 2H), 3.11-2.68 (m, 4H), 2.39-2.14 (m, 1H), 2.01-1.82 (m, 7H), 1.62-1.38 (m, 3H), 1.25-0.50 (m, 9H). m/z: ES⁺ [M+H]⁺=507.64.

### Example 5.02: ((1S,5R)-6-(6-(2-(5-cyclopropylpyrimidin-4-yl)-4-fluorophenoxy)-1,2,4-triazin-5-yl)-2,6-diazabicyclo[3.2.1]octan-2-yl)((S)-2-methylpyrrolidin-2-yl)methanone

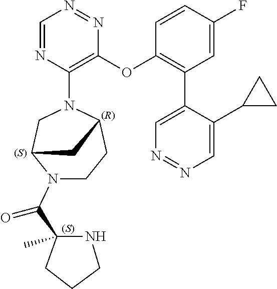

Colourless solid (29 mg, 17% over 3 steps).

¹H NMR (400 MHz, DMSO-d₆, 295 K) δ 8.95-8.93 (m, 1H), 8.45-8.41 (m, 2H), 8.30 (s, 1H), 7.59-7.56 (m, 1H), 7.49-7.44 (m, 2H), 5.30-5.17 (m, 1H), 4.77-4.69 (m, 2H), 4.12 (s, 1H), 3.98-3.74 (m, 1H), 3.57-3.53 (m, 1H), 3.20-3.11 (s, 1H), 2.99-2.57 (m, 1H), 2.23 (s, 1H), 2.07-1.65 (m, 8H), 1.40 (s, 3H), 0.75 (s, 4H). m/z: ES⁺ [M+H]⁺=531.26.

### Example 5.03: ((1S,5R)-6-(6-(2-(5-cyclopropylpyrimidin-4-yl)-4-fluorophenoxy)-1,2,4-triazin-5-yl)-2,6-diazabicyclo[3.2.1]octan-2-yl)((S)-2-methylpyrrolidin-2-yl)methanone

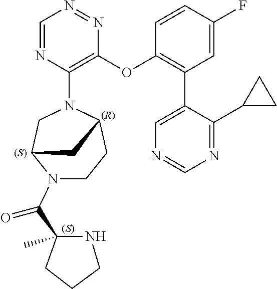

Colourless solid (30 mg, 8% over 3 steps).

¹H NMR (400 MHz, DMSO-d₆, 295 K) δ 8.92 (s, 1H), 8.87 (s, 1H), 8.50 (s, 1H), 8.44 (s, 1H), 8.39 (s, 1H), 7.71 (s, 1H), 7.60-7.51 (t, 2H), 5.24-4.45 (t, 2H), 4.45-3.60 (m, 4H), 3.25-3.02 (t, 2H), 2.62 (s, 1H), 2.23 (s, 1H), 2.23-1.62 (m, 7H), 1.68-1.51 (m, 3H), 1.38-0.80 (m, 3H), 0.80-0.22 (s, 1H). m/z: ES⁺ [M+H]⁺=513.27.

### Example 5.04: N,N-dicyclopropyl-5-fluoro-2-((5-((1S,5R)-2-((S)-2-methylpyrrolidine-2-carbonyl)-2,6-diazabicyclo[3.2.1]octan-6-yl)-1,2,4-triazin-6-yl)oxy)benzamide

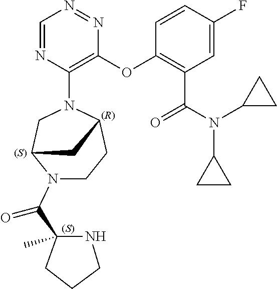

Colourless solid, formic acid salt (26 mg, 30% over 3 steps).

¹H NMR (400 MHz, DMSO-d₆, 295 K) δ 8.61-8.50 (m, 1H), 8.29 (s, 1H), 7.51-7.29 (m, 3H), 5.26 (s, 1H), 5.08-4.75 (m, 1H), 4.33-4.15 (m, 1H), 4.16-3.92 (m, 3H), 3.68-3.47 (m, 1H), 3.36-2.93 (m, 2H), 2.86-2.71 (m, 1H), 2.69-2.56 (m, 1H), 2.32-2.16 (m, 1H), 1.98-1.81 (m, 2H), 1.88-1.60 (m, 5H), 1.48-1.32 (m, 3H), 0.82-0.41 (m, 8H).

m/z: ES⁺ [M+H]⁺=535.62.

### Example 5.05: N,N-dimethyl-5-fluoro-2-((5-((1S,5R)-2-((S)-2-methylpyrrolidine-2-carbonyl)-2,6-diazabicyclo[3.2.1]octan-6-yl)-1,2,4-triazin-6-yl)oxy)benzamide

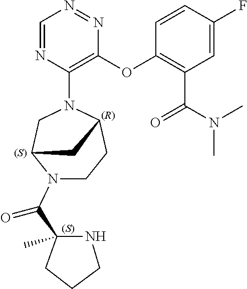

Colourless solid, formic acid salt (26 mg, 30% over 3 steps).

¹H NMR (400 MHz, DMSO-d₆, 295 K) δ 8.61-8.50 (m, 1H), 8.29 (s, 1H), 7.51-7.29 (m, 3H), 5.26 (s, 1H), 5.08-4.75 (m, 1H), 4.33-4.15 (m, 1H), 4.16-3.92 (m, 3H), 3.68-3.47 (m, 1H), 3.36-2.93 (m, 2H), 2.86-2.71 (m, 1H), 2.69-2.56 (m, 1H), 2.32-2.16 (m, 1H), 1.98-1.81 (m, 2H), 1.88-1.60 (m, 5H), 1.48-1.32 (m, 3H), 0.82-0.41 (m, 8H).

m/z: ES⁺ [M+H]⁺=535.62.

### Example 5.06: N-ethyl-5-fluoro-N-isopropyl-2-((4-((1S,5R)-2-((S)-2-methylpyrrolidine-2-carbonyl)-2,6-diazabicyclo[3.2.1]octan-6-yl)pyrimidin-5-yl)oxy)benzamide

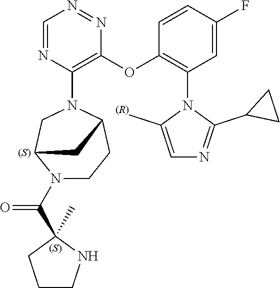

Colourless solid, (26 mg, 17% over 3 steps).

¹H NMR (400 MHz, DMSO-d₆, 300 K) δ 8.62-8.22 (m, 1H), 7.81-7.60 (m, 2H), 7.59-7.35 (m, H), 6.75-6.38 (m, 1H), 5.80-4.92 (m, 1H), 4.90-4.60 (m, 1H), 4.59-4.03 (m, H), 3.93-3.63 (m, H), 3.62-3.42 (m, 1H), 3.18-2.92 (m, H), 2.90-2.77 (m, 2H), 2.76-2.55 (m, 2H), 2.38-2.15 (m, 1H), 2.03-1.79 (m, 5H), 1.77-1.50 (m, 5H), 1.31-1.16 (m, 3H), 1.15-0.91 (m, 5H), 0.89-0.74 (m, 1H). m/z: ES⁺ [M+H]⁺=535.25.

### Example 5.07: ((1S,5R)-6-(6-(2-(2,5-dimethyl-1H-imidazol-1-yl)-4-fluorophenoxy)-1,2,4-triazin-5-yl)-2,6-diazabicyclo[3.2.1]octan-2-yl)((S)-2-methylpyrrolidin-2-yl)methanone

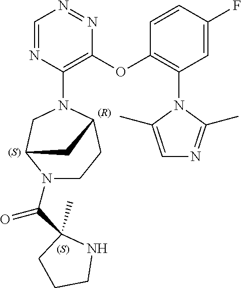

Colourless solid, trifluoroacetate salt (11 mg, 9% over 3 steps).

¹H NMR (400 MHz, DMSO-d₆, 300 K) δ 8.47 (d, J=21.2 Hz, 1H), 7.80-7.40 (m, 3H), 6.65-6.37 (m, 1H), 5.60-4.98 (m, 1H), 4.80-4.58 (m, 1H), 4.57-4.03 (m, 1H), 4.02-3.75 (m, 1H), 3.61-3.52 (m, 1H), 2.95-2.79 (m, 3H), 2.71-2.63 (m, 1H), 2.55-2.53 (m, 1H), 2.30-2.11 (m, 1H), 2.08-1.95 (m, 3H), 1.95-1.81 (m, 4H), 1.78-1.52 (m, 5H), 1.39-1.13 (m, 3H). m/z: ES⁺ [M+H]⁺=507.25.

### Example 5.08: N-(5-fluoro-2-((5-((1S,5R)-2-((S)-2-methylpyrrolidine-2-carbonyl)-2,6-diazabicyclo[3.2.1]octan-6-yl)-1,2,4-triazin-6-yl)oxy)phenyl)-N-methylisobutyramide

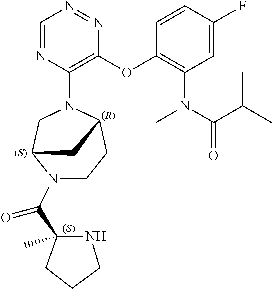

Colourless solid, trifluoroacetate salt (54 mg, 39% over 3 steps).

¹H NMR (400 MHz, DMSO-d₆, 300 K) δ 8.56-8.46 (m, 1H), 7.56-7.24 (m, 3H), 5.70-5.13 (m, 1H), 5.12-4.72 (m, 1H), 4.71-4.22 (m, 2H), 4.21-3.96 (m, 1H), 3.95-3.52 (m, 2H), 3.25-2.96 (m, 1H), 2.90-2.78 (m, 1H), 2.74-2.68 (m, 1H), 2.68-2.58 (m, 1H), 2.57-2.52 (m, 2H), 2.32-2.17 (m, 1H), 2.10-1.74 (m, 3H), 1.74-1.47 (m, 4H), 1.35-1.20 (m, 3H), 1.18-0.61 (m, 6H). m/z: ES⁺ [M+H]⁺=512.25.

### Example 5.09: ((1S,5R)-6-(6-(2-(2-cyclopropylpyridin-3-yl)-4-fluorophenoxy)-1,2,4-triazin-5-yl)-2,6-diazabicyclo[3.2.1]octan-2-yl)((S)-2-methylpyrrolidin-2-yl)methanone

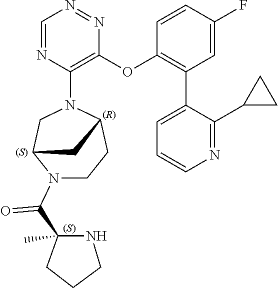

Colourless solid (13 mg, 8% over 3 steps).

¹H NMR (400 MHz, DMSO-d₆, 295 K) δ 8.60-8.20 (m, 2H), 7.56-7.40 (m, 1H), 7.40-7.32 (m, 3H), 7.20-7.00 (m, 1H), 5.96-4.98 (m, 1H), 4.94-4.70 (m, 1H), 4.60-3.70 (m, 1H), 3.60-3.50 (m, 1H), 2.80-2.70 (m, 1H), 2.70-2.68 (m, 2H), 2.40-2.30 (m, 2H), 2.00-1.60 (m, 8H), 1.40-1.20 (m, 4H), 1.00-0.70 (m, 3H). m/z: ES⁺ [M+H]⁺=530.25.

### Example 5.10: ((1S,5R)-6-(6-(2-(2-cyclopropylpyridin-3-yl)-4-fluorophenoxy)-1,2,4-triazin-5-yl)-2,6-diazabicyclo[3.2.1]octan-2-yl)((S)-2-methylpyrrolidin-2-yl)methanone

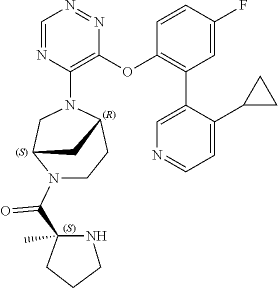

Colourless solid (68 mg, 19% over 3 steps).

¹H NMR (400 MHz, DMSO-d₆, 295 K) δ 8.42-8.30 (m, 2H), 8.21-8.09 (m, 1H), 7.63-7.49 (m, 1H), 7.48-7.32 (m, 2H), 6.90-6.68 (m, 1H), 5.58-5.00 (m, 1H), 4.84-4.00 (m, 2H), 3.93-3.62 (m, 1H), 3.59-3.43 (m, 1H), 2.94-2.78 (m, 2H), 2.71-2.68 (m, 2H), 2.30-2.12 (m, 1H), 2.10-1.72 (m, 2H), 1.72-1.35 (m, 6H), 1.35-1.15 (m, 3H), 1.08-0.81 (m, 1H), 0.81-0.45 (m, 3H). m/z: ES⁺ [M+H]⁺=530.25.

### Example 5.11: ((1S,5R)-6-(6-(2-(cyclopropylmethoxy)-4-fluorophenoxy)-1,2,4-triazin-5-yl)-2,6-diazabicyclo[3.2.1]octan-2-yl)((S)-2-methylpyrrolidin-2-yl)methanone

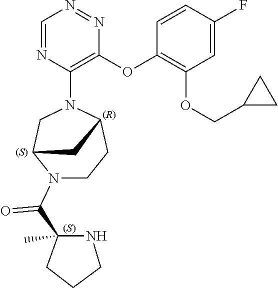

Colourless solid (52 mg, 37% over 3 steps).

¹H NMR (400 MHz, DMSO-d₆, 295 K) δ 8.51 (d, J=11.8 Hz, 1H), 7.33 (dt,J=8.7, 5.1 Hz, 1H), 7.05 (dd, J=10.7, 2.9 Hz, 1H), 6.82 (td, J=8.5, 2.9 Hz, 1H), 5.41 (d, J=113.3 Hz, 1H), 5.20-4.73 (m, 1H), 4.42 (d, J=59.8 Hz, 1H), 4.27-3.97 (m, 1H), 3.89-3.55 (m, 3H), 3.33 (s, 2H), 2.84 (q, J=6.1, 5.5 Hz, 1H), 2.63 (h, J=7.3 Hz, 1H), 2.25 (d, J=11.5 Hz, 1H), 2.06 (s, 2H), 1.85-1.43 (m, 5H), 1.28 (d, J=6.3 Hz, 3H), 1.02-0.86 (m, 1H), 0.38 (t, J=7.6 Hz, 2H), 0.08 (dq, J=14.7, 5.5, 5.0 Hz, 2H). m/z: ES⁺ [M+H]⁺=483.25.

### Example 5.12: (5-fluoro-2-((5-((1S,5R)-2-((S)-2-methylpyrrolidine-2-carbonyl)-2,6-diazabicyclo[3.2.1]octan-6-yl)-1,2,4-triazin-6-yl)oxy)phenyl)(pyrrolidin-1-yl)methanone

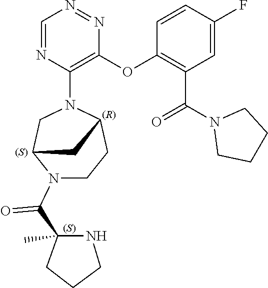

Colourless solid (11 mg, 14% over 3 steps).

¹H NMR (400 MHz, DMSO-d₆, 295 K) δ 8.80-8.46 (m, 1H), 7.55-7.25 (m, 3H), 5.92-5.01 (m, 1H), 5.09-4.80 (m, 1H), 4.70-4.44 (m, 1H), 4.42-4.04 (m, 1H), 3.86-3.50 (m, 1H), 3.25-3.05 (m, 5H), 2.87-2.75 (m, 1H), 2.67-2.55 (m, 1H), 2.3-2.18 (m, 1H), 2.11-1.89 (m, 2H), 1.85-1.55 (m, 10H), 1.22-1.38 (m, 3H). m/z: ES⁺ [M+H]⁺=510.25.

### Example 5.13: (5-fluoro-2-((5-((1S,5R)-2-((S)-2-methylpyrrolidine-2-carbonyl)-2,6-diazabicyclo[3.2.1]octan-6-yl)-1,2,4-triazin-6-yl)oxy)phenyl)(morpholino)methanone

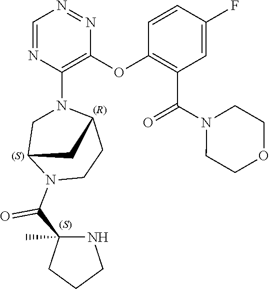

Colourless solid, TFA salt (12 mg, 12% over 3 steps).

¹H NMR (400 MHz, DMSO-d₆, 295 K) δ 9.20-9.06 (s, 1H), 8.80-8.66 (s, 1H), 8.60-8.46 (m, 1H), 7.65-7.45 (m, 3H), 5.30-5.07 (m, 1H), 4.80-4.66 (m, 1H), 4.46-3.80 (m, 2H), 3.65-3.45 (m, 6H), 3.25-3.15 (m, 4H), 3.11-3.05 (m, 2H), 2.27-2.17 (m, 2H), 2.16-1.78 (m, 6H), 1.68-1.59 (m, 3H), 1.34-1.24 (m, 1H). m/z: ES⁺ [M+H]⁺=526.25.

### Example 5.14: ((1S,5R)-6-(6-(2-(3,3-dimethylmorpholine-4-carbonyl)-4-fluorophenoxy)-1,2,4-triazin-5-yl)-2,6-diazabicyclo[3.2.1]octan-2-yl)((S)-2-methylpyrrolidin-2-yl)methanone

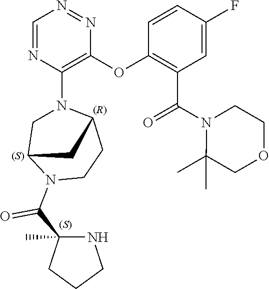

Colourless solid, TFA salt (35 mg, 15% over 3 steps).

¹H NMR (400 MHz, DMSO-d₆, 295 K) δ 9.36-9.28 (m, 1H), 8.98-8.79 (m, 1H), 8.70-8.60 (m, 1H), 7.51-7.00 (m, 3H), 5.32-5.06 (m, 1H), 4.94-4.69 (m, 1H), 4.42-4.19 (m, 1H), 3.90-3.81 (m, 2H), 3.44-2.93 (m, 8H), 2.40-1.70 (m, 9H), 1.68-1.55 (m, 3H), 1.44-0.88 (m, 6H). m/z: ES⁺ [M+H]⁺=554.30.

### Example 5.15: ((1S,5R)-6-(6-(2-((1S,4S)-2-oxa-5-azabicyclo[2.2.1]heptane-5-carbonyl)-4-fluorophenoxy)-1,2,4-triazin-5-yl)-2,6-diazabicyclo[3.2.1]octan-2-yl)((S)-2-methylpyrrolidin-2-yl)methanone

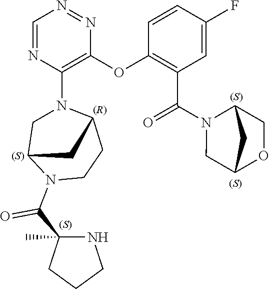

Colourless solid, formic acid salt (24 mg, 18% over 3 steps).

¹H NMR (400 MHz, DMSO-d₆, 295 K) δ 8.65-8.42 (m, 1H), 8.40-8.10 (s, 1H), 7.60-7.32 (m, 3H), 5.40-5.25 (m, 1H), 5.20-5.03 (m, 1H), 4.89-4.62 (m, 1H), 4.60-4.42 (m, 1H), 4.40-4.05 (m, 3H), 3.92-3.85 (m, 1H), 3.39-3.25 (m, 2H), 3.20-3.05 (m, 2H), 2.98-2.70 (m, 3H), 2.35-2.05 (m, 1H), 2.05-1.92 (m, 2H), 1.85-1.62 (m, 6H), 1.60-1.35 (m, 4H). m/z: ES⁺ [M+H]⁺=538.30.

### Example 5.16: ((1S,5R)-6-(6-(2-((1R,4R)-2-oxa-5-azabicyclo[2.2.1]heptane-5-carbonyl)-4-fluorophenoxy)-1,2,4-triazin-5-yl)-2,6-diazabicyclo[3.2.1]octan-2-yl)((S)-2-methylpyrrolidin-2-yl)methanone

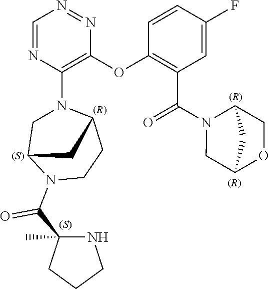

Colourless solid(36 mg, 21% over 3 steps).

¹H NMR (400 MHz, DMSO-d₆, 300 K) δ 9.07-8.85 (m, 1H), 8.65-8.52 (m, 1H), 8.36-8.22 (m, 1H), 8.14-7.75 (m, 1H), 7.52-7.41 (m, 1H), 7.38-7.31 (m, 1H), 7.18-6.98 (m, 1H), 5.54-4.95 (m, 1H), 4.94-3.96 (m, 2H), 3.84-3.59 (m, 1H), 3.58-3.45 (m, 1H), 3.02-2.76 (m, 2H), 2.62-2.55 (m, 1H), 2.22-2.13 (m, 1H), 1.97-1.84 (m, 1H), 1.83-1.75 (m, 1H), 1.74-1.35 (m, 6H), 1.34-1.14 (m, 3H), 1.13-0.85 (m, 5H). m/z: ES⁺ [M+H]⁺=538.30.

### Example 5.17: ((1S,5R)-6-(6-(2-((3S,5S)-3,5-dimethylmorpholine-4-carbonyl)-4-fluorophenoxy)-1,2,4-triazin-5-yl)-2,6-diazabicyclo[3.2.1]octan-2-yl)((S)-2-methylpyrrolidin-2-yl)methanone

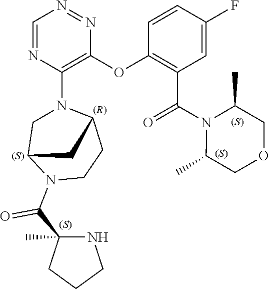

Colourless solid, formic acid salt (49 mg, 27% over 3 steps).

¹H NMR (400 MHz, DMSO-d₆, 295 K) δ 8.59-8.50 (m, 1H), 8.32-8.21 (m, 1H), 7.53-7.36 (m, 3H), 5.45-5.20 (m, 1H), 5.15-5.03 (m, 1H), 4.86-4.75 (m, 1H), 4.37-4.23 (m, 1H), 4.16-4.03 (m, 1H), 3.94-3.48 (m, 5H), 3.45-3.05 (m, 3H), 2.99-2.90 (m, 1H), 2.82-2.70 (m, 1H), 2.31-2.19 (m, 1H), 2.12-1.58 (m, 7H), 1.46-1.31 (m, 3H), 1.26-0.98 (m, 6H). m/z: ES⁺ [M+H]⁺=554.20.

### Example 5.18: ((1S,5R)-6-(6-(2-(2-cyclopropyl-5-methyl-1H-imidazol-1-yl)-4-fluorophenoxy)-1,2,4-triazin-5-yl)-2,6-diazabicyclo[3.2.1]octan-2-yl)((S)-2-methylpyrrolidin-2-yl)methanone

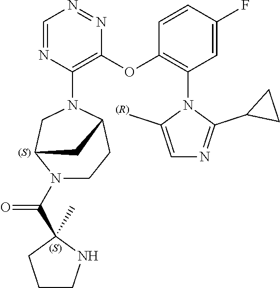

Colourless solid (35 mg, 26% over 3 steps).

¹H NMR (400 MHz, DMSO-d₆, 297 K) δ 8.60-8.30 (m, 1H), 7.81-7.59 (m, 2H), 7.59-7.42 (m, 1H), 6.61-6.25 (m, 1H), 5.75-4.95 (m, 1H), 4.90-4.63 (m, 1H), 4.60-4.03 (m, 1H), 4.02-3.64 (m, 1H), 3.64-3.40 (m, 1H), 3.20-2.54 (m, 4H), 2.39-2.13 (m, 1H), 2.09-1.79 (m, 5H), 1.68-1.48 (m, 5H), 1.47-1.32 (m, 1H), 1.28 (s, 3H), 0.79-0.37 (m, 4H). m/z: ES⁺ [M+H]⁺=533.20.

### Example 5.19: ((1S,5R)-6-(6-(2-(1-cyclopropyl-1H-imidazol-5-yl)-4-fluorophenoxy)-1,2,4-triazin-5-yl)-2,6-diazabicyclo[3.2.1]octan-2-yl)((S)-2-methylpyrrolidin-2-yl)methanone

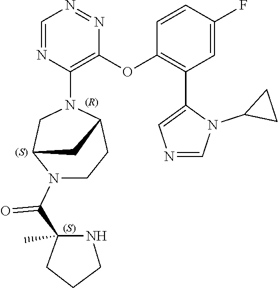

Light green solid, formic acid salt (42 mg, 14% over 3 steps).

¹H NMR (400 MHz, DMSO-d₆, 300 K) δ 9.92-9.62 (m, 1H), 8.95-8.78 (m, 1H), 8.58-8.12 (m, 2H), 7.84-7.01 (m, 4H), 5.44-4.55 (m, 2H), 4.44-3.52 (m, 4H), 3.23-2.81 (m, 2H), 2.41-1.59 (m, 11H), 1.57-0.64 (m, 4H). m/z: ES⁺ [M+H]⁺=519.20.

### Example 5.20: 6-cyclopropyl-1-(5-fluoro-2-((5-((1S,5R)-2-((S)-2-methylpyrrolidine-2-carbonyl)-2,6-diazabicyclo[3.2.1]octan-6-yl)-1,2,4-triazin-6-yl)oxy)phenyl)pyridin-2(1H)-one

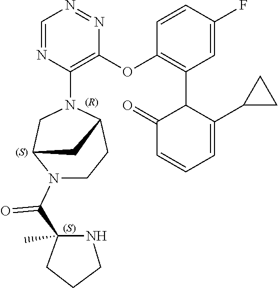

Colourless solid (20 mg, 39% over 3 steps).

¹H NMR (400 MHz, DMSO-d₆, 300 K) δ 8.61-8.45 (m, 1H), 7.78-7.60 (m, 1H), 7.52-7.39 (m, 1H), 7.38-7.28 (m, 1H), 7.28-7.10 (m, 1H), 7.10-7.00 (m, 1H), 6.90-6.77 (m, 1H), 5.68-5.15 (m, 1H), 5.14-4.73 (m, 1H), 4.61-4.28 (m, 1H), 4.28-3.58 (m, 2H), 3.31-3.08 (m, 1H), 2.98-2.75 (m, 1H), 2.75-2.58 (m, 1H), 2.35-2.15 (m, 1H), 2.08-1.88 (m, 3H), 1.88-1.70 (m, 2H), 1.70-1.50 (m, 4H), 1.39-1.15 (m, 3H), 0.88-0.70 (m, 2H), 0.70-0.38 (m, 2H). m/z: ES⁺ [M+H]⁺=546.25.

### Example 5.21: ((1S,5R)-6-(6-(4-fluoro-2-(5-methoxy-2-methyl-1H-imidazol-1-yl)phenoxy)-1,2,4-triazin-5-yl)-2,6-diazabicyclo[3.2.1]octan-2-yl)((S)-2-methylpyrrolidin-2-yl)methanone

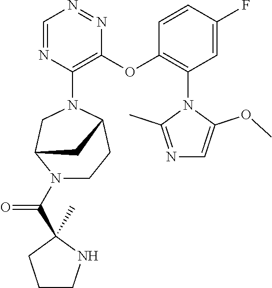 [SMILES: COc1cnc(C)n1-c1cc(F)ccc1Oc1nncnc1N1C[C@@H]2C[C@H]1CCN2C(=O)[C@]1(C)CCCN1]

Colourless solid (27 mg, 32% over 3 steps).

¹H NMR (400 MHz, DMSO-d₆, 297 K) δ 8.52-8.40 (m, 1H), 7.71-7.55 (m, 2H), 7.55-7.46 (m, 1H), 6.14-5.98 (m, 1H), 5.64-5.14 (m, 1H), 4.87-4.65 (m, 1H), 4.62-3.70 (m, 3H), 3.67-3.48 (m, 4H), 2.91-2.80 (m, 1H), 2.72-2.59 (m, 1H), 2.33-2.19 (m, 1H), 2.04-1.82 (m, 5H), 1.82-1.49 (m, 6H), 1.36-1.22 (m, 3H). m/z: ES⁺ [M+H]⁺=523.45.

### Example 5.22: ((1S,5R)-6-(6-(4-fluoro-2-(5-isopropoxy-2-methyl-1H-imidazol-1-yl)phenoxy)-1,2,4-triazin-5-yl)-2,6-diazabicyclo[3.2.1]octan-2-yl)((S)-2-methylpyrrolidin-2-yl)methanone

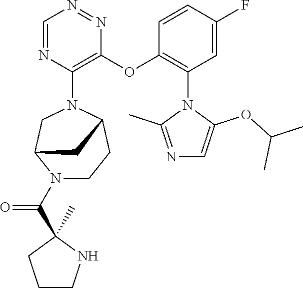 [SMILES: Cc1ncc(OC(C)C)n1-c1cc(F)ccc1Oc1nncnc1N1C[C@@H]2C[C@H]1CCN2C(=O)[C@]1(C)CCCN1]

Colourless solid (20 mg, 30% over 3 steps).

¹H NMR (400 MHz, DMSO-d₆, 297 K) δ 8.55-8.39 (m, 1H), 7.71-7.56 (m, 2H), 7.55-7.45 (m, 1H), 6.20-6.03 (m, 1H), 5.65-5.10 (m, 1H), 4.97-4.70 (m, 1H), 4.60-4.00 (m, 2H), 4.00-3.50 (m, 2H), 3.22-2.92 (m, 1H), 2.90-2.57 (m, 3H), 2.32-2.18 (m, 1H), 2.05-1.79 (m, 5H), 1.76-1.48 (m, 5H), 1.35-1.20 (m, 3H), 1.07-0.86 (m, 6H). m/z: ES⁺ [M+H]⁺=551.15.

### Example 5.23: ((1S,5R)-6-(6-(2-(2,5-dicyclopropyl-1H-imidazol-1-yl)-4-fluorophenoxy)-1,2,4-triazin-5-yl)-2,6-diazabicyclo[3.2.1]octan-2-yl)((S)-2-methylpyrrolidin-2-yl)methanone

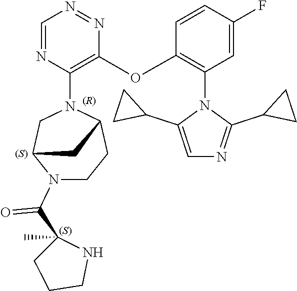

Colourless solid, formic acid salt (28 mg, 14% over 3 steps).

¹H NMR (400 MHz, DMSO-d₆, 300 K) δ 8.59-8.41 (m, 1H), 8.40-8.10 (m, 1H), 7.80-7.61 (m, 2H), 7.60-7.50 (m, 1H), 6.49-6.31 (m, 1H), 5.15-5.00 (m, 1H), 4.80-4.70 (m, 1H), 4.50-3.45 (m, 3H), 3.40-2.52 (m, 3H), 2.40-2.17 (m, 1H), 2.10-1.50 (m, 7H), 1.49-1.17 (m, 5H), 0.80-0.21 (m, 8H). m/z: ES⁺ [M+H]⁺=559.30.

### Example 5.24: ((1S,5R)-6-(6-(2-(4,6-dicyclopropylpyrimidin-5-yl)-4-fluorophenoxy)-1,2,4-triazin-5-yl)-2,6-diazabicyclo[3.2.1]octan-2-yl)((S)-2-methylpyrrolidin-2-yl)methanone

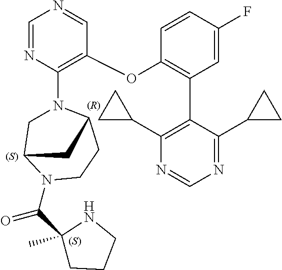

Colourless solid, trifluoroacetate salt (4 mg, 9% over 3 steps).

¹H NMR (400 MHz, DMSO-d₆, 300 K) δ 8.88-8.70 (m, 1H), 8.66-8.56 (m, 1H), 8.50-8.30 (m, 1H), 7.34-7.25 (m, 1H), 7.24-7.15 (m, 1H), 5.30-5.07 (m, 1H), 5.00-4.66 (m, 1H), 4.06-3.60 (m, 4H), 3.05-2.95 (m, 1H), 2.85-2.65 (m, 1H), 2.11-2.05 (m, 2H), 2.03-1.97 (m, 2H), 1.94-1.87 (m, 4H), 1.84-1.69 (m, 3H), 1.68-1.59 (m, 1H), 1.14-0.94 (m, 2H). 0.94-0.87 (m, 4H). 0.76-0.66 (m, 1H). 0.22-0.16 (m, 3H). m/z: ES⁺ [M+H]⁺=571.35.

### Example 5.25: N,N-dimethyl-5-fluoro-2-((5-((1S,5R)-2-((S)-2-methylpyrrolidine-2-carbonyl)-2,6-diazabicyclo[3.2.1]octan-6-yl)-1,2,4-triazin-6-yl)oxy)benzamide

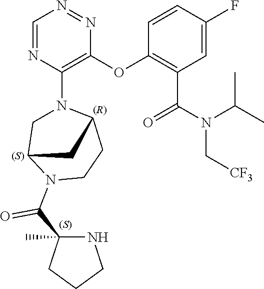

Colourless solid (27 mg, 18% over 3 steps).

¹H NMR (400 MHz, DMSO-d₆, 295 K) δ 8.53-8.46 (m, 1H), 7.50-7.36 (m, 3H), 5.30-5.17 (m, 1H), 5.10-4.96 (m, 1H), 4.86-4.70 (m, 1H), 4.66-4.26 (m, 3H), 4.24-3.95 (m, 3H), 3.85-3.80 (m, 1H), 3.65-3.50 (m, 1H), 2.95-2.83 (m, 1H), 2.78-2.65 (m, 1H), 2.36-2.22 (m, 1H), 2.10-1.69 (m, 3H), 1.66-1.55 (m, 4H). 1.34-1.24 (m, 3H). m/z: ES⁺ [M+H]⁺=580.45.

### Example 5.26: ((1S,5R)-6-(6-(2-(4-cyclopropyl-6-methylpyrimidin-5-yl)-4-fluorophenoxy)-1,2,4-triazin-5-yl)-2,6-diazabicyclo[3.2.1]octan-2-yl)((S)-2-methylpyrrolidin-2-yl)methanone

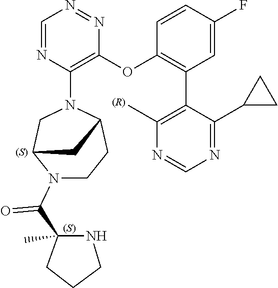

Colourless solid (63 mg, 14% over 3 steps).

¹H NMR (400 MHz, DMSO-d₆, 300 K) δ 8.80-8.72 (m, 1H), 8.52-8.43 (m, 1H), 8.23 (s, 1H), 7.61-7.55 (m, 1H), 7.47-7.42 (m, 2H), 5.16-5.08 (m, 1H), 4.82-4.4.50 (m, 2H), 4.15-4.11 (m, 2H), 3.92-3.89 (m, 1H), 3.71-3.59 (m, 1H), 3.56-3.50 (m, 1H), 3.25-2.90 (m, 2H), 2.82-2.77 (m, 1H), 2.23-2.14 (m, 4H), 1.98-1.81 (m, 4H), 1.75-1.71 (m, 1H), 1.61-1.59 (m, 2H), 1.38-1.23 (m, 3H), 0.93-0.91 (m, 3H), 0.74-0.70 (m, 1H). m/z: ES⁺ [M+H]⁺=545.15.

### Example 5.27: ((1S,5R)-6-(6-(4-fluoro-2-(5-methyl-2-(trifluoromethyl)-1H-imidazol-1-yl)phenoxy)-1,2,4-triazin-5-yl)-2,6-diazabicyclo[3.2.1]octan-2-yl)((S)-2-methylpyrrolidin-2-yl)methanone

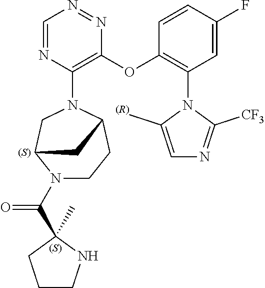

Colourless solid, trifluoroacetic acid salt (28 mg, 15% over 3 steps).

¹H NMR (400 MHz, DMSO-d₆, 300 K) δ 8.60-8.46 (m, 1H), 7.82-7.74 (m, 1H), 7.74-7.67 (m, 1H), 7.66-7.53 (m, 1H), 7.09-6.90 (m, 1H), 5.35-5.07 (m, 1H), 4.80-4.48 (m, 1H), 4.31-4.10 (m, 1H), 3.98-3.74 (m, 2H), 3.72-3.63 (m, 1H), 3.62-3.58 (m, 1H), 3.18-3.09 (m, 1H), 3.00-2.90 (m, 1H), 2.88-2.71 (m, 1H), 2.30-2.13 (m, 1H), 2.04-1.89 (m, 4H), 1.89-1.50 (m, 5H), 1.37 (s, 3H). m/z: ES⁺ [M+H]⁺=561.15.

### Example 5.28: ((1S,5R)-6-(6-(2-(2-cyclopropyl-5-methoxy-1H-imidazol-1-yl)-4-fluorophenoxy)-1,2,4-triazin-5-yl)-2,6-diazabicyclo[3.2.1]octan-2-yl)((S)-2-methylpyrrolidin-2-yl)methanone

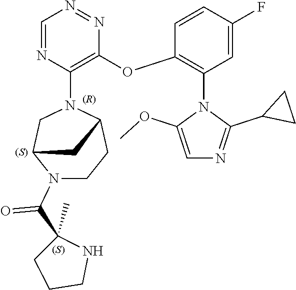

Colourless solid (16 mg, 12% over 3 steps).

¹H NMR (400 MHz, DMSO-d₆, 297 K) δ 8.68-8.45 (m, 1H), 8.27 (s, 1H), 7.77-7.59 (m, 2H), 7.54 (d, J=8.5 Hz, 1H), 6.07 (d, J=14.3 Hz, 1H), 5.24 (s, 1H), 4.73 (d, J=16.4 Hz, 1H), 4.25 (dd, J=30.6, 11.3 Hz, 1H), 3.94-3.80 (m, 1H), 3.69 (s, 3H), 3.13 (d, J=40.0 Hz, 2H), 2.96 (dt, J=11.5, 6.0 Hz, 1H), 2.81-2.68 (m, 1H), 2.28 (q, J=14.2, 12.3 Hz, 1H), 2.01-1.65 (m, 7H), 1.38 (s, 4H), 0.70 (q, J=7.6 Hz, 2H), 0.59-0.31 (m, 2H). m/z: ES⁺ [M+H]⁺=549.25.

### Example 5.29: ((1S,5R)-6-(6-(4-fluoro-2-(2,4,5-trimethyl-1H-imidazol-1-yl)phenoxy)-1,2,4-triazin-5-yl)-2,6-diazabicyclo[3.2.1]octan-2-yl)((S)-2-methylpyrrolidin-2-yl)methanone

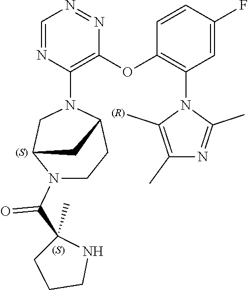

Colourless solid (40 mg, 29% over 3 steps).

¹H NMR (400 MHz, CDCl₃, 300 K) δ 8.54 (m, 1H), 8.38 (m, 1H), 7.45 (m, 1H), 7.35-7.30 (m, 1H), 7.15-7.09 (m, 1H), 5.45-5.30 (m, 1H), 4.95-4.80 (m, 1H), 4.60-4.30 (m, 9H), 4.00-3.87 (m, 2H), 3.73-3.70 (m, 1H), 3.60-3.50 (m, 1H), 3.25-3.15 (m, 1H), 3.05-2.95 (m, 1H), 2.30-2.15 (m, 3H), 2.12-2.00 (m, 4H), 1.92-1.82 (m, 2H), 1.81-1.74 (m, 2H). m/z: ES⁺ [M+H]⁺=521.20.

### Example 5.30: ((1S,5R)-6-(6-(2-(2,5-dicyclopropyl-1H-pyrrol-1-yl)-4-fluorophenoxy)-1,2,4-triazin-5-yl)-2,6-diazabicyclo[3.2.1]octan-2-yl)((S)-2-methylpyrrolidin-2-yl)methanone

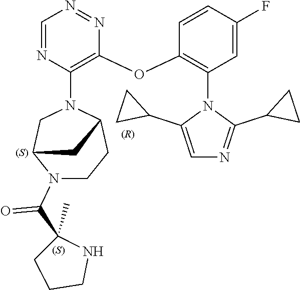

Colourless solid (36 mg, 11% over 3 steps).

¹H NMR (400 MHz, DMSO-d₆, 300 K) δ 8.51-8.46 (m, 1H), 7.67 (s, 1H), 7.56-7.48 (m, 2H), 5.57-5.50 (m, 1H), 5.46-5.37 (m, 1H), 5.12 (s, 1H), 4.72-4.23 (m, 2H), 3.92-3.47 (m, 2H), 2.84 (s, 2H), 2.65-2.63 (m, 1H), 2.22-2.20 (m, 1H), 1.89-1.81 (m, 2H), 1.68-1.59 (m, 6H), 1.28 (s, 5H), 0.59-0.52 (m, 2H), 0.49-0.13 (m, 6H). m/z: ES⁺ [M+H]⁺=558.25.

### Example 5.31: ((1S,5R)-6-(6-(2-(4-cyclopropyl-2-methylpyridin-3-yl)-4-fluorophenoxy)-1,2,4-triazin-5-yl)-2,6-diazabicyclo[3.2.1]octan-2-yl)((S)-2-methylpyrrolidin-2-yl)methanone

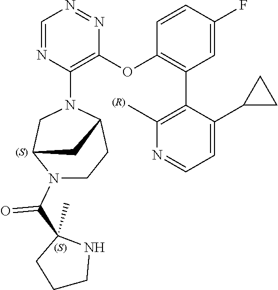

Colourless solid (13 mg, 16% over 3 steps).

¹H NMR (400 MHz, DMSO-d₆, 300 K) δ 8.62-8.45 (m, 1H), 8.42-8.09 (m, 2H), 7.69-7.51 (m, 1H), 7.51-7.42 (m, 1H), 7.42-7.11 (m, 1H), 6.78-6.41 (m, 1H), 5.17-5.08 (m, 1H), 4.86-4.67 (m, 1H), 4.43-3.71 (m, 2H), 3.71-3.23 (m, 1H), 3.23-2.62 (m, 3H), 5.58-2.42 (m, 1H), 2.32-2.10 (m, 4H), 2.10-1.72 (m, 5H), 1.72-1.21 (m, 6H), 1.09-0.84 (m, 1H), 0.81-0.28 (m, 3H). m/z: ES⁺ [M+H]⁺=544.30.

### Example 5.32: 5-cyclopropyl-6-(5-fluoro-2-((5-((1S,5R)-2-((S)-2-methylpyrrolidine-2-carbonyl)-2,6-diazabicyclo[3.2.1]octan-6-yl)-1,2,4-triazin-6-yl)oxy)phenyl)-1-methylpyridin-2(1H)-one

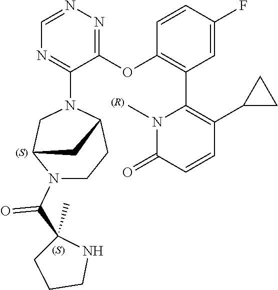

Colourless solid (13 mg, 9% over 3 steps).

¹H NMR (400 MHz, DMSO-d₆, 300K) δ 8.55-8.37 (m, 1H), 7.62-7.60 (m, 1H), 7.56-7.46 (m, 2H), 6.98-6.94 (m, 1H), 6.35-6.31 (m, 1H), 5.32-5.10 (m, 1H), 4.96-4.70 (m, 1H), 4.47-4.05 (m, 1H), 4.00-3.73 (m, 1H), 3.58-3.54 (m, 2H), 3.10-3.07 (m, 3H), 2.83-2.68 (m, 2H), 2.67-2.58 (m, 1H), 2.23-2.21 (m, 1H), 1.95-1.89 (m, 2H), 1.76-1.36 (m, 6H), 1.25-1.22 (m, 2H), 1.21-1.14 (m, 1H), 0.71-0.57 (m, 1H), 0.54-0.29 (m, 3H). m/z: ES⁺ [M+H]⁺=560.30.

### Example 5.33: ((1S,5R)-6-(6-(2-(3,5-dicyclopropyl-4H-1,2,4-triazol-4-yl)-4-fluorophenoxy)-1,2,4-triazin-5-yl)-2,6-diazabicyclo[3.2.1]octan-2-yl)((S)-2-methylpyrrolidin-2-yl)methanone

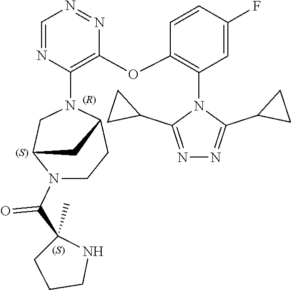

Colourless solid (31 mg, 17% over 3 steps).

¹H NMR (400 MHz, DMSO-d₆, 300K) δ 8.60-8.35 (m, 1H), 7.98-7.70 (m, 2H), 7.68-7.55 (m, 1H), 5.65-5.04 (m, 1H), 4.85-4.70 (m, 1H), 4.55-3.83 (m, 2H), 3.82-3.56 (m, 2H), 3.19-2.90 (m, 1H), 2.90-2.79 (m, 1H), 2.78-2.59 (m, 1H), 2.40-2.18 (m, 1H), 2.01-1.80 (m, 2H), 1.80-1.56 (m, 4H), 1.56-1.40 (m, 3H), 1.28 (s, 3H), 1.00-0.41 (m, 8H). m/z: ES⁺ [M+H]⁺=560.25.

### Example 5.34: ((1S,5R)-6-(6-(2-(3,5-diethyl-4H-1,2,4-triazol-4-yl)-4-fluorophenoxy)-1,2,4-triazin-5-yl)-2,6-diazabicyclo[3.2.1]octan-2-yl)((S)-2-methylpyrrolidin-2-yl)methanone

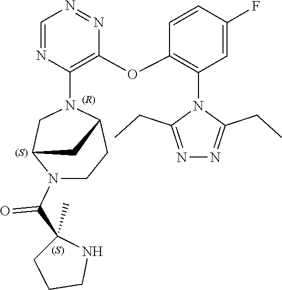

Colourless solid (39 mg, 20% over 3 steps).

¹H NMR (400 MHz, DMSO-d₆, 300K) δ 8.60-8.24 (m, 1H), 7.81-7.76 (m, 1H), 7.76-7.66 (m, 1H), 7.65-7.57 (m, 1H), 5.83-5.06 (m, 1H), 4.80-4.62 (m, 1H), 4.62-4.05 (m, 1H), 3.99-3.70 (m, 1H), 3.69-3.48 (m, 1H), 3.24-2.69 (m, 3H), 2.68-2.59 (m, 1H), 2.50-2.35 (m, 4H), 2.31-2.19 (m, 1H), 2.00-1.84 (m, 1H), 1.83-1.38 (m, 6H), 1.28 (s, 3H), 1.16-1.00 (m, 6H). m/z: ES⁺ [M+H]⁺=536.25.

### Example 5.35: ((1S,5R)-6-(6-(2-(5-cyclopropyl-2-(trifluoromethyl)-1H-imidazol-1-yl)-4-fluoro phenoxy)-1,2,4-triazin-5-yl)-2,6-diazabicyclo[3.2.1]octan-2-yl)((S)-2-ethylpyrrolidin-2-yl)methanone

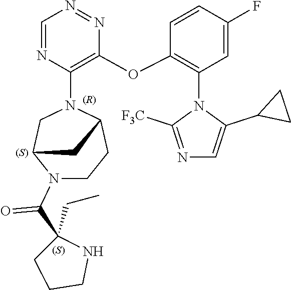

Colourless solid (27 mg, 13% over 3 steps).

¹H NMR (400 MHz, DMSO-d₆, 300K) δ 8.68-8.32 (m, 1H), 7.85-7.72 (m, 1H), 7.72-7.63 (m, 1H), 7.63-7.50 (m, 1H), 7.31-7.01 (m, 1H), 5.25 (br s, 1H), 4.89-4.47 (m, 1H), 4.39-4.12 (m, 1H), 3.91-3.75 (m, 1H), 3.63-3.50 (m, 2H), 3.16-2.99 (m, 1H), 2.86-2.83 (m, 1H), 2.63-2.60 (m, 1H), 2.26-2.05 (s, 1H), 1.95-1.73 (m, 3H), 1.71-1.35 (m, 7H), 0.89-0.62 (m, 5H), 0.62-0.41 (m, 2H). m/z: ES⁺ [M+H]⁺=601.25.

### Example 5.36: ((1S,5R)-6-(6-(2-(5-cyclopropyl-2-methyl-1H-imidazol-1-yl)-4-fluorophenoxy)-1,2,4-triazin-5-yl)-2,6-diazabicyclo[3.2.1]octan-2-yl)((S)-2-ethylpyrrolidin-2-yl)methanone

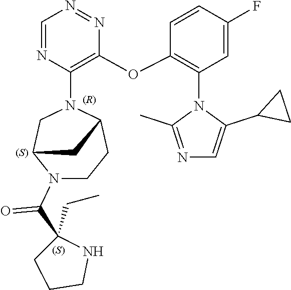

Colourless solid (37 mg, 26% over 3 steps).

¹H NMR (400 MHz, DMSO-d₆, 300 K) δ 8.52-8.46 (m, 1H), 7.68-7.60 (m, 2H), 7.55-7.51 (m, 1H), 6.46-6.34 (m, 1H), 5.35-5.25 (m, 1H), 4.74-4.59 (m, 1H), 4.27-4.26 (m, 1H), 3.92-3.90 (m, 1H), 3.77-3.51 (m, 1H), 3.20-2.80 (m, 3H), 2.74-2.62 (m, 2H), 2.30-2.16 (m, 1H), 2.04-1.88 (m, 3H), 1.78-1.75 (m, 1H), 1.75-1.60 (m, 8H), 1.42-1.31 (m, 1H), 0.78 (m, 3H), 0.66-0.43 (m, 3H), 0.43-0.38 (m, 1H). m/z: ES⁺ [M+H]⁺=547.30.

### Example 5.37: ((1S,5R)-6-(6-(2-(5-cyclopropyl-3-methylisoxazol-4-yl)-4-fluorophenoxy)-1,2,4-triazin-5-yl)-2,6-diazabicyclo[3.2.1]octan-2-yl)((S)-2-ethylpyrrolidin-2-yl)methanone

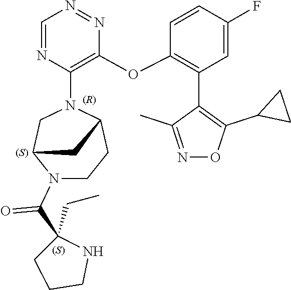

Off-white solid (16 mg, 23% over 3 steps).

¹H NMR (400 MHz, DMSO-d₆, 300K) δ 8.58-8.32 (m 1H), 7.65-7.47 (m, 1H), 7.44-7.24 (m, 2H), 5.48-5.07 (m, 1H), 4.99-4.60 (m, 1H), 4.42-4.02 (m, 2H), 3.89-3.73 (m, 1H), 2.85-2.75 (m, 2H), 2.64-2.57 (m, 1H), 2.24-2.07 (m, 2H), 2.05-1.86 (m, 4H), 1.82-1.47 (m, 9H), 0.97-0.59 (m, 7H). m/z: ES⁺ [M+H]⁺=548.20.

### Example 5.38: ((1S,5R)-6-(6-(4-fluoro-2-(1-isopropyl-4-methyl-1H-1,2,3-triazol-5-yl)phenoxy)-1,2,4-triazin-5-yl)-2,6-diazabicyclo[3.2.1]octan-2-yl)((S)-2-methylpyrrolidin-2-yl)methanone

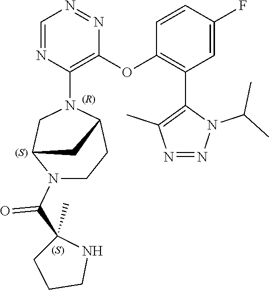

Colourless solid, bis(formic acid) salt (62 mg, 22% over 3 steps).

¹H NMR (400 MHz, DMSO-d₆, 300K) δ 8.69-8.40 (m, 1H), 8.47 (s, 2H), 7.85-7.30 (m, 3H), 5.25-5.18 (m, 1H), 4.92-4.59 (m, 1H), 4.50-4.00 (m, 2H), 3.99-3.68 (m, 1H), 3.67-3.45 (m, 1H), 3.36-2.65 (m, 3H), 2.35-2.18 (m, 1H), 2.02 (s, 3H), 1.98-1.51 (m, 8H), 1.50-1.32 (m, 6H), 1.30-1.20 (m, 3H). m/z: ES⁺ [M+H]⁺=536.25.

### Example 5.39: ((1S,5R)-6-(6-(2-(3-cyclopropyl-5-methyl-4H-1,2,4-triazol-4-yl)-4-fluorophenoxy)-1,2,4-triazin-5-yl)-2,6-diazabicyclo[3.2.1]octan-2-yl)((S)-2-methylpyrrolidin-2-yl)methanone

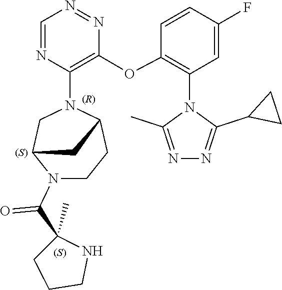

Colourless solid (26 mg, 13% over 3 steps).

¹H NMR (400 MHz, DMSO-d₆, 300K) δ 8.60-8.35 (m, 1H), 7.89-7.70 (m, 2H), 7.68-7.55 (m, 1H), 5.77-5.00 (m, 1H), 5.00-4.65 (m, 1H), 4.60-4.20 (m, 1H), 4.05-3.55 (m, 2H), 3.20-2.70 (m, 3H), 2.66-2.59 (m, 1H), 2.30-2.18 (m, 1H), 2.18-2.02 (m, 3H), 2.02-1.40 (m, 8H), 1.38-1.18 (m, 3H), 0.90-0.60 (m, 4H). m/z: ES⁺ [M+H]⁺=534.25.

### Example 5.40: ((1S,5R)-6-(6-(4-fluoro-2-(1-isopropyl-4-methyl-1H-pyrazol-5-yl)phenoxy)-1,2,4-triazin-5-yl)-2,6-diazabicyclo[3.2.1]octan-2-yl)((S)-2-methylpyrrolidin-2-yl)methanone

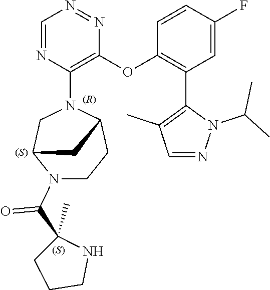

Light yellow solid, HCl salt (38 mg, 26% over 3 steps).

¹H NMR (400 MHz, DMSO-d₆, 300 K) δ 9.80-9.50 (m, 1H), 9.00-8.80 (m, 1H), 7.72-7.51 (m, 1H), 7.49-7.32 (m, 2H), 7.25-7.10 (m, 2H), 4.92-4.50 (m, 1H), 4.12-3.90 (m, 1H), 3.85-3.50 (m, 2H), 3.40-3.15 (m, 2H), 3.12-2.56 (m, 3H), 2.32-2.15 (m, 3H), 2.10-1.82 (m, 5H), 1.80-1.52 (m, 2H), 1.50-1.42 (m, 2H), 1.37-1.30 (m, 2H), 1.35-1.00 (m, 6H). m/z: ES⁺ [M+H]⁺=535.25.

### Example 5.41: ((1S,5R)-6-(6-(2-(2-cyclopropyl-5-isopropoxy-1H-imidazol-1-yl)-4-fluorophenoxy)-1,2,4-triazin-5-yl)-2,6-diazabicyclo[3.2.1]octan-2-yl)((S)-2-methylpyrrolidin-2-yl)methanone

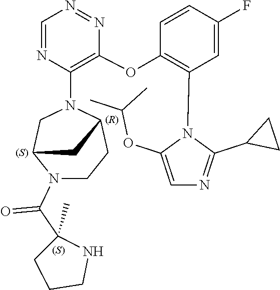

Colourless oil (11 mg, 32% over 3 steps).

¹H NMR (400 MHz, CDCl₃, 300K) δ 8.65-8.45 (m, 1H), 7.65-7.45 (m, 2H), 7.25-6.70 (m, 2H), 6.25-5.90 (m, 1H), 5.60-5.10 (m, 1H), 5.00-4.60 (m, 1H), 4.12-4.0 (m, 1H), 4.0-3.8 (m, 1H), 3.8-3.3 (m, 2H) 3.25-2.85 (m, 2H), 2.65-2.20 (m, 1H), 2.20-1.95 (m, 6H), 1.80-1.70 (m, 2H), 1.70-1.60 (m, 2H), 1.50-1.40 (m, 2H), 1.20-1.10 (m, 4H), 1.05-0.90 (m, 1H), 0.90-0.70 (m, 3H), 0.60-0.40 (m, 1H), 0.10-0.05 (m, 1H).

m/z: ES⁺ [M+H]⁺=577.25.

### Example 5.42: ((S)-2-ethylpyrrolidin-2-yl)((1S,5R)-6-(6-(4-fluoro-2-(5-(1-fluorocyclopropyl)-2-methyl-1H-imidazol-1-yl)phenoxy)-1,2,4-triazin-5-yl)-2,6-diazabicyclo[3.2.1]octan-2-yl)methanone

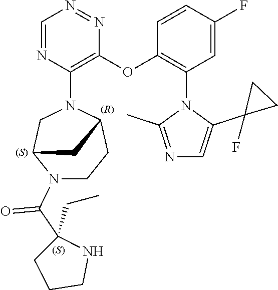

Colourless solid (3 mg, 2% over 3 steps).

¹H NMR (400 MHz, DMSO-d₆, 300K) 68.57-8.44 (m, 1H), 7.78-7.43 (m, 3H), 7.15-6.85 (m, 1H), 5.24-5.19 (m, 1H), 4.67-4.59 (m, 1H), 4.23 (s, 1H), 3.77 (s, 1H), 3.53 (s, 1H), 3.13-2.72 (m, 2H), 2.15 (s, 1H), 2.12-1.80 (m, 5H), 1.60-1.54 (m, 8H), 1.01-0.94 (m, 4H), 0.78-0.74 (m, 4H). m/z: ES⁺ [M+H]⁺=565.30.

### Example 5.43: ((1S,5R)-6-(6-(2-(4-cyclopropyl-1-methyl-1H-pyrazol-5-yl)-4-fluorophenoxy)-1,2,4-triazin-5-yl)-2,6-diazabicyclo[3.2.1]octan-2-yl)((S)-2-methylpyrrolidin-2-yl)methanone

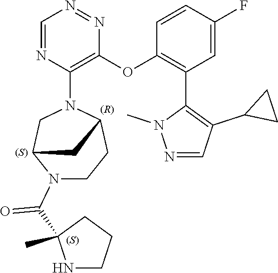

Colourless solid (50 mg, 12% over 3 steps).

¹H NMR (400 MHz, DMSO-d₆, 300K) δ 8.73-8.23 (m, 1H), 7.83-7.27 (m, 3H), 7.20-6.81 (m, 1H), 5.65-5.12 (m, 1H), 5.71-4.98 (m, 1H), 4.96-4.52 (m, 1H), 4.52-4.05 (m, 1H), 4.03-3.53 (m, 2H), 3.50-3.35 (m, 3H), 3.35-3.30 (m, 1H), 3.21-2.78 (m, 2H), 2.75-2.63 (m, 1H), 2.35-2.24 (m, 1H), 2.15-1.80 (m, 2H), 1.77-1.42 (m, 5H), 1.27 (s, 4H), 0.75-0.65 (m, 1H), 0.56-0.14 (m, 3H). m/z: ES⁺ [M+H]⁺=533.20.

### Example 5.44: ((1S,5R)-6-(6-(4-fluoro-2-(4-isopropyl-1-methyl-1H-pyrazol-5-yl)phenoxy)-1,2,4-triazin-5-yl)-2,6-diazabicyclo[3.2.1]octan-2-yl)((S)-2-methylpyrrolidin-2-yl)methanone

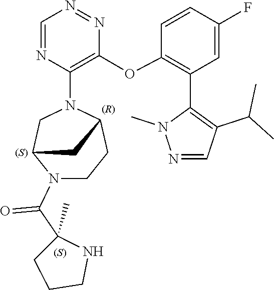

Colourless solid (53 mg, 17% over 3 steps).

¹H NMR (400 MHz, DMSO-d₆, 300K) δ 8.62-8.30 (m, 1H), 7.71-7.56 (m, 1H), 7.55-7.45 (m, 1H), 7.45-7.11 (m, 2H), 7.57-5.00 (m, 1H), 4.85-4.51 (m, 1H), 4.50-4.02 (m, 1H), 4.00-3.65 (m, 1H), 4.62-3.36 (m, 4H), 3.30-2.81 (m, 3H), 2.80-2.68 (m, 1H), 2.46-2.35 (m, 1H), 2.31-2.14 (m, 1H), 1.99-1.80 (m, 2H), 1.78-1.45 (m, 5H), 1.40-1.22 (m, 3H), 1.09-0.76 (m, 6H). m/z: ES⁺ [M+H]⁺=535.25

### Example 5.45: ((S)-2-ethylpyrrolidin-2-yl)((1S,5R)-6-(6-(4-fluoro-2-(5-isopropyl-3-methylisoxazol-4-yl)phenoxy)-1,2,4-triazin-5-yl)-2,6-diazabicyclo[3.2.1]octan-2-yl)methanone

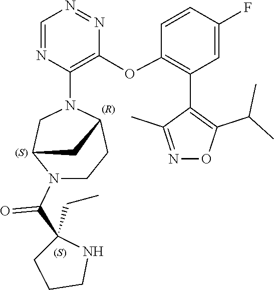

Colourless solid, HCl salt (35 mg, 23% over 3 steps)

¹H NMR (400 MHz, DMSO-d₆, 300K) δ 9.75 (s, 1H), 9.04-8.83 (m, 2H), 7.76-7.54 (m, 1H), 7.54-7.27 (m, 2H), 5.35-5.23 (m, 1H), 5.15-5.80 (m, 2H), 4.32-4.14 (m, 1H), 4.06-3.88 (m, 1H), 3.85-3.67 (m, 1H), 3.55-3.32 (m, 1H), 3.04-2.87 (m, 2H), 2.43-1.70 (m, 13H), 1.29-0.97 (m, 6H), 0.97-0.78 (m, 3H). m/z: ES⁺ [M+H]⁺=550.30.

### Example 5.46: ((1S,5R)-6-(6-(4-fluoro-2-(4-isopropyl-1-methyl-1H-imidazol-5-yl)phenoxy)-1,2,4-triazin-5-yl)-2,6-diazabicyclo[3.2.1]octan-2-yl)((S)-2-methylpyrrolidin-2-yl)methanone

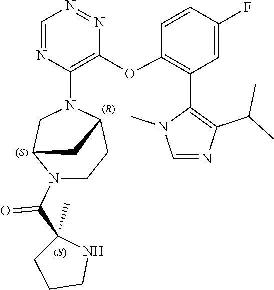

Colourless solid (7 mg, 4% over 3 steps).

¹H NMR (400 MHz, DMSO-d₆, 300K) δ 8.49-8.43 (m, 1H), 7.52-7.30 (m, 4H), 5.47-5.19 (m, 1H), 4.83-4.24 (m, 2H), 3.95-3.76 (m, 1H), 3.10-3.05 (m, 3H), 2.83-2.54 (m, 4H), 2.25-2.04 (m, 2H), 1.94-1.85 (m, 1H), 1.82-1.62 (m, 5H), 1.56-1.49 (m, 3H), 1.38-1.35 (m, 3H), 1.34-0.95 (m, 3H), 0.84-0.80 (m, 2H). m/z: ES⁺ [M+H]⁺=535.30.

### Example 5.47: ((1S,5R)-6-(6-(2-(3-(ethyl(isopropyl)amino)oxetan-3-yl)-4-fluorophenoxy)-1,2,4-triazin-5-yl)-2,6-diazabicyclo[3.2.1]octan-2-yl)((S)-2-methylpyrrolidin-2-yl)methanone

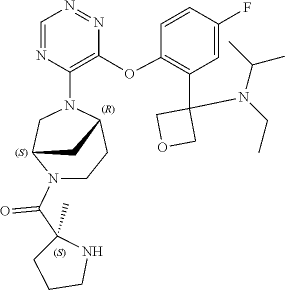

Colourless solid (17 mg, 8% over 3 steps)

¹H NMR (400 MHz, DMSO-d₆, 300K) δ 8.50-8.32 (m, 1H), 8.09-7.88 (m, 1H), 7.75-7.59 (m, 1H), 7.12-6.91 (m, 1H), 5.22-5.00 (m, 3H), 5.00-4.70 (m, 3H), 4.70-4.47 (m, 1H), 4.20-3.75 (m, 2H), 3.08-2.98 (m, 5H), 2.87-2.69 (m, 2H), 2.18-1.93 (m, 1H), 1.93-1.80 (m, 2H), 1.78-1.67 (m, 5H), 1.67-1.42 (m, 3H), 0.89-0.63 (m, 9H). m/z: ES⁺ [M+H]⁺=554.30.

### Example 5.48: ((1S,5R)-6-(6-(4-fluoro-2-(1,3,4-trimethyl-1H-pyrazol-5-yl)phenoxy)-1,2,4-triazin-5-yl)-2,6-diazabicyclo[3.2.1]octan-2-yl)((S)-2-methylpyrrolidin-2-yl)methanone

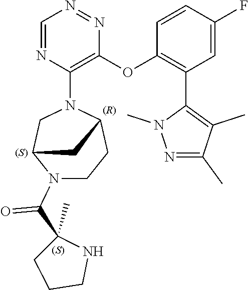

Colourless solid (27 mg, 24% over 3 steps).

¹H NMR (400 MHz, DMSO-d₆, 300K) δ 8.55-8.45 (m, 1H), 8.30 (s, 2H), 7.55-7.35 (m, 4H), 5.35-5.05 (m, 1H), 4.71-4.65 (m, 1H), 4.31-4.24 (m, 2H), 3.95-3.77 (m, 3H), 3.15-2.84 (m, 3H), 2.24-2.00 (m, 3H), 1.90-1.88 (m, 1H), 1.72-1.54 (m, 6H), 1.38-1.35 (m, 1H), 1.31-1.25 (m, 2H), 1.04-1.02 (m, 5H). m/z: ES⁺ [M+H]⁺=521.20.

### Example 5.49: ((1S,5R)-6-(6-(2-(4-cyclopropyl-1,3-dimethyl-1H-pyrazol-5-yl)-4-fluorophenoxy)-1,2,4-triazin-5-yl)-2,6-diazabicyclo[3.2.1]octan-2-yl)((S)-2-methylpyrrolidin-2-yl)methanone

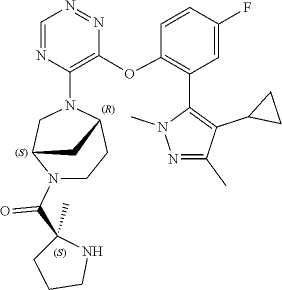

Colourless solid (23 mg, 20% over 3 steps).

¹H NMR (400 MHz, DMSO-d₆, 300K) δ 8.53-8.40 (m, 1H), 8.24 (s, 1H), 7.55-7.35 (m, 3H), 5.36-5.20 (m, 1H), 4.72-4.64 (m, 1H), 4.52-4.10 (m, 2H), 3.90-3.85 (m, 1H), 3.77-3.55 (m, 3H), 3.39-3.10 (m, 1H), 2.95-2.70 (m, 2H), 2.42-2.02 (m, 4H), 1.95-1.69 (m, 5H), 1.65-1.40 (m, 1H), 1.38-1.32 (m, 2H), 1.04-0.65 (m, 4H), 0.51-0.43 (m, 2H), 0.40-0.18 (m, 2H). m/z: ES⁺ [M+H]⁺=547.20.

### Example 5.50: ((1S,5R)-6-(6-(2-(1-cyclopropyl-4-methyl-1H-pyrazol-5-yl)-4-fluorophenoxy)-1,2,4-triazin-5-yl)-2,6-diazabicyclo[3.2.1]octan-2-yl)((S)-2-methylpyrrolidin-2-yl)methanone

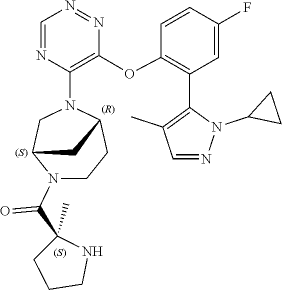

Colourless solid (21 mg, 4% over 3 steps).

¹H NMR (400 MHz, DMSO-d₆, 300K) δ 8.50-8.46 (m, 1H), 7.59 (s, 1H), 7.54-7.33 (m, 2H), 7.22-7.14 (m, 1H), 5.36-5.25 (m, 1H), 4.80-4.61 (m, 1H), 4.25 (s, 2H), 3.99-3.69 (m, 2H), 3.58-3.45 (m, 1H), 3.08-2.70 (m, 2H), 2.78-2.60 (m, 1H), 2.32-2.24 (m, 1H), 2.08-1.40 (m, 10H), 1.32 (s, 3H), 0.88 (s, 2H), 0.75 (s, 1H), 0.56 (s, 1H). m/z: ES⁺ [M+H]⁺=533.20.

### Example 5.51: ((1S,5R)-6-(6-(4-fluoro-2-(5-isopropyl-1,3-dimethyl-1H-pyrazol-4-yl)phenoxy)-1,2,4-triazin-5-yl)-2,6-diazabicyclo[3.2.1]octan-2-yl)((S)-2-methylpyrrolidin-2-yl)methanone

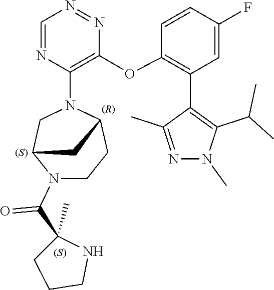

Off-white solid (124 mg, 28% over 3 steps).

¹H NMR (400 MHz, DMSO-d₆, 300 K) δ 8.50-8.45 (m, 1H), 7.48-6.95 (m, 3H), 5.95-4.95 (m, 2H), 4.70-4.25 (m, 2H), 4.95-3.56 (m, 5H), 3.18-2.84 (m, 3H), 2.62-2.25 (m, 2H), 2.05-1.59 (m, 10H), 1.45-1.28 (m, 3H), 1.07-0.92 (m, 6H). m/z: ES⁺ [M+H]⁺=549.25.

### Example 5.52: ((1S,5R)-6-(6-(4-fluoro-2-(3-isopropyl-1,5-dimethyl-1H-pyrazol-4-yl)phenoxy)-1,2,4-triazin-5-yl)-2,6-diazabicyclo[3.2.1]octan-2-yl)((S)-2-methylpyrrolidin-2-yl)methanone

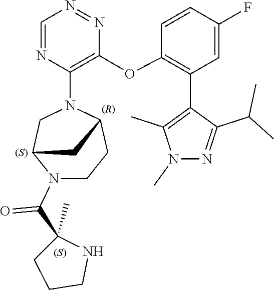

Colourless solid (102 mg, 18% over 3 steps).

¹H NMR (400 MHz, DMSO-d₆, 300 K) δ 8.62-8.29 (m, 1H), 7.65-7.42 (m, 1H), 7.40-7.20 (m, 1H), 7.20-6.98 (m, 1H), 5.75-4.98 (m, 1H), 4.89-4.54 (m, 1H), 4.56-4.06 (m, 1H), 5.05-3.71 (m, 1H), 3.71-3.48 (m, 4H), 3.18-2.75 (m, 2H), 2.69-2.51 (m, 2H), 2.31-2.16 (m, 1H), 2.02-1.74 (m, 5H), 1.74-1.45 (m, 5H), 1.45-1.21 (m, 4H), 1.12-0.78 (m, 6H). m/z: ES⁺ [M+H]⁺=549.30.

### Example 5.53: ((1S,5R)-6-(6-(2-(3,5-diisopropyl-1H-pyrazol-4-yl)-4-fluorophenoxy)-1,2,4-triazin-5-yl)-2,6-diazabicyclo[3.2.1]octan-2-yl)((S)-2-methylpyrrolidin-2-yl)methanone

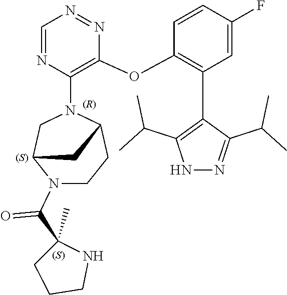

Light orange solid, HCl salt (156 mg, 62% over 3 steps).

¹H NMR (400 MHz, DMSO-d₆, 300 K) δ 12.00-11.50 (br s, 1H), 9.21-8.81 (m, 1H), 8.03-7.55 (m, 2H), 7.55-7.32 (m, 1H), 7.32-7.18 (m, 1H), 5.45-4.62 (m, 2H), 4.37-4.09 (m, 1H), 4.02-3.97 (m, 2H), 3.85-3.67 (m, 2H), 3.51-2.87 (m, 3H), 2.87-2.63 (m, 2H), 2.41-2.19 (m, 2H), 2.18-1.77 (m, 5H), 1.72-1.53 (m, 3H), 1.441-0.68 (m, 12H). m/z: ES⁺ [M+H]⁺=563.20.

### Example 5.54 ((S)-2-ethylpyrrolidin-2-yl)((1S,5R)-6-(6-(4-fluoro-2-(5-(1-hydroxycyclopropyl)-2-methyl-1H-imidazol-1-yl)phenoxy)-1,2,4-triazin-5-yl)-2,6-diazabicyclo[3.2.1]octan-2-yl)methanone

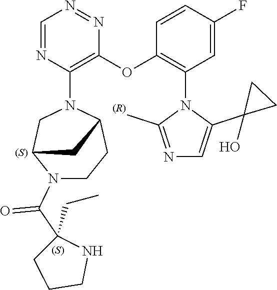

Colourless solid (7 mg, 10% over 3 steps).

¹H NMR (400 MHz, DMSO-d₆, 300K) 68.57-8.44 (m, 1H), 7.78-7.43 (m, 3H), 7.15-6.85 (m, 1H), 5.24-5.19 (m, 1H), 4.67-4.59 (m, 1H), 4.23 (s, 1H), 3.77 (s, 1H), 3.53 (s, 1H), 3.13-2.72 (m, 2H), 2.15 (s, 1H), 2.12-1.80 (m, 5H), 1.60-1.54 (m, 8H), 1.01-0.94 (m, 4H), 0.78-0.74 (m, 4H). m/z: ES⁺ [M+H]⁺=565.30.

### Examples 5.55 and 5.56: atropisomers of ((1S,5R)-6-(6-(4-fluoro-2-(2-isopropyl-4,5-dimethyl-1H-imidazol-1-yl)phenoxy)-1,2,4-triazin-5-yl)-2,6-diazabicyclo[3.2.1]octan-2-yl)((S)-2-methylpyrrolidin-2-yl)methanone

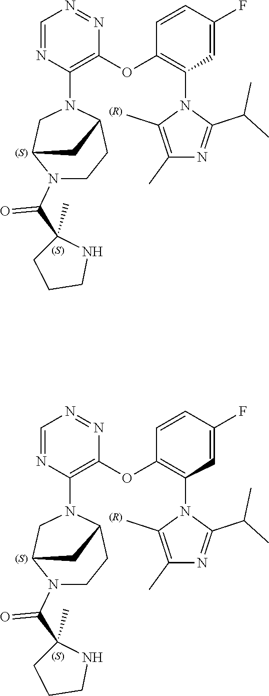

Separation by chiral HPLC gave two single stable atropisomers:

Peak #1 (structure shown on left above): Rt1=28.25 min, colourless solid (27 mg, 3%).

¹H NMR (400 MHz, DMSO-d₆, 300 K) δ 8.52-8.49 (m, 1H), 7.67-7.51 (m, 3H), 5.59-5.19 (m, 1H), 4.90-4.72 (m, 1H), 4.43-4.30 (m, 1H), 3.78-3.75 (m, 1H), 3.59-3.56 (m, 1H), 3.16-2.86 (m, 4H), 2.85-2.64 (m, 2H), 2.46-2.25 (m, 1H), 1.97-1.93 (m, 4H), 1.80-1.72 (m, 4H), 1.72-1.60 (m, 4H), 1.28 (s, 3H), 1.12-0.82 (m, 6H).

m/z: ES⁺ [M+H]⁺=549.30.

Peak #2 (structure shown on right above): Rt2=33.48 min, colourless solid (22 mg, 2%).

¹H NMR (400 MHz, DMSO-d₆, 300 K) δ 8.52-8.49 (m, 1H), 7.67-7.51 (m, 3H), 5.59-5.19 (m, 1H), 4.90-4.72 (m, 1H), 4.43-4.30 (m, 1H), 3.78-3.75 (m, 1H), 3.59-3.56 (m, 1H), 3.16-2.86 (m, 4H), 2.85-2.64 (m, 2H), 2.46-2.25 (m, 1H), 1.97-1.93 (m, 4H), 1.80-1.72 (m, 4H), 1.72-1.60 (m, 4H), 1.28 (s, 3H), 1.12-0.82 (m, 6H).

m/z: ES⁺ [M+H]⁺=549.30.

### Example 5.57: ((1S,5R)-6-(6-(4-fluoro-2-(5-isopropyl-2-methyl-1H-imidazol-1-yl)phenoxy)-1,2,4-triazin-5-yl)-2,6-diazabicyclo[3.2.1]octan-2-yl)((S)-2-methylpyrrolidin-2-yl)methanone

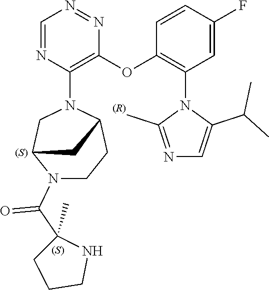

Brown solid, formic acid salt (11 mg, 13%).

¹H NMR (400 MHz, DMSO-d₆, 300 K) δ 8.61-8.42 (m, 1H), 8.29 (s, 1H), 7.81-7.62 (m, 2H), 7.62-7.45 (m, 1H), 6.73-6.37 (m, 1H), 5.28-4.09 (m, 3H), 3.99-3.76 (m, 1H), 3.68-3.57 (m, 1H), 3.57-2.58 (m, 4H), 2.41-2.12 (m, 3H), 2.09-1.80 (m, 7H), 1.79-1.42 (m, 5H), 1.12-0.80 (m, 6H). m/z: ES⁺ [M+H]⁺=535.25.

### Examples 5.58 and 5.59: atropisomers of 6-(5-fluoro-2-((5-((1S,5R)-2-((S)-2-methylpyrrolidine-2-carbonyl)-2,6-diazabicyclo[3.2.1]octan-6-yl)-1,2,4-triazin-6-yl)oxy)phenyl)-1-isopropyl-5-methylpyridin-2(1H)-one

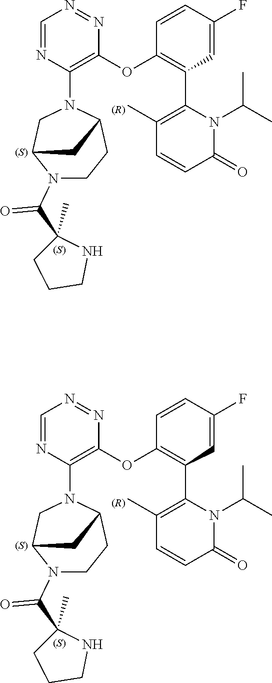

Separation by chiral HPLC gave two single stable atropisomers:

Peak #1: Rt1=6.09 min, colourless solid (47 mg, 4%). ¹H NMR (400 MHz, DMSO-d₆, 300 K) δ 8.54-8.45 (m, 1H), 7.69-7.05 (m, 4H), 6.36-6.18 (m, 1H), 5.75-5.05 (m, 1H), 4.95-4.17 (m, 2H), 3.97-3.45 (m, 3H), 3.15-2.68 (m, 3H), 2.45-2.15 (m, 2H), 1.95-1.67 (m, 10H), 1.45-1.05 (m, 9H). m/z: ES⁺ [M+H]⁺=562.30.

Peak #2: Rt2=10.79 min, colourless solid (58 mg, 5%).

¹H NMR (400 MHz, DMSO-d₆, 300 K) δ 8.54-8.45 (m, 1H), 7.69-7.05 (m, 4H), 6.36-6.18 (m, 1H), 5.75-5.05 (m, 1H), 4.95-4.17 (m, 2H), 3.97-3.45 (m, 3H), 3.15-2.68 (m, 3H), 2.45-2.15 (m, 2H), 1.95-1.67 (m, 10H), 1.45-1.05 (m, 9H). m/z: ES⁺ [M+H]⁺=562.30.

### Examples 5.60 and 5.61: atropisomers of ((S)-2-ethylpyrrolidin-2-yl)((1S,5R)-6-(6-(4-fluoro-2-(2-isopropyl-4,5-dimethyl-1H-imidazol-1-yl)phenoxy)-1,2,4-triazin-5-yl)-2,6-diazabicyclo[3.2.1]octan-2-yl)methanone

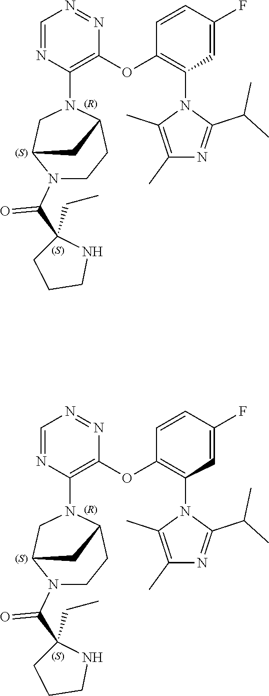

Mixture isolated as a yellow solid, bis(formic acid) salt (167 mg, 15% over 3 steps).

¹H NMR (400 MHz, DMSO-d₆, 300 K) δ 8.58-8.38 (m, 1H), 8.23 (s, 2H), 7.91-7.38 (m, 3H), 5.36-4.89 (m, 2H), 4.85-4.75 (m, 1H), 4.41-4.12 (m, 1H), 3.97-3.80 (m, 2H), 3.79-3.40 (m, 1H), 3.37-3.03 (m, 2H), 2.90-2.80 (m, 1H), 2.35-2.27 (m, 1H), 2.01-1.92 (m, 5H), 1.91-1.34 (m, 10H), 1.37-0.47 (m, 9H). m/z: ES⁺ [M+H]⁺=563.30.

Separation by chiral HPLC gave two single stable atropisomers:

Peak #1: Rt1=8.02 min, colourless solid (43 mg, 44%).

¹H NMR (400 MHz, DMSO-d₆, 300 K) δ 8.52-8.49 (m, 1H), 8.29 (s, 1H), 7.67-7.61 (m, 1H), 7.63-7.48 (m, 2H), 5.35-5.23 (m, 1H), 4.74-4.71 (m, 1H), 4.30-4.17 (m, 1H), 3.95-3.77 (m, 1H), 3.72-3.54 (m, 1H), 3.35-3.02 (m, 1H), 2.90-2.78 (m, 1H), 2.66-2.54 (m, 2H), 2.27-2.18 (m, 1H), 2.02-1.78 (m, 6H), 1.85-1.65 (m, 9H), 1.17-1.15 (m, 1H), 1.09-0.84 (m, 6H), 0.82-0.76 (m, 3H). m/z: ES⁺ [M+H]⁺=563.35.

Peak #2: Rt2=11.25 min, colourless solid (31 mg, 29%).

¹H NMR (400 MHz, DMSO-d₆, 300 K) δ 8.53-8.48 (m, 1H), 8.35 (s, 2H), 7.70-7.66 (m, 1H), 7.61-7.59 (m, 1H), 7.56-7.51 (m, 1H), 5.33-5.23 (m, 1H), 4.33-4.21 (m, 1H), 3.93-3.79 (m, 1H), 3.65-3.52 (m, 1H), 3.29-3.22 (m, 1H), 3.02-2.86 (m, 1H), 2.70-2.59 (m, 1H), 2.35-2.17 (m, 1H), 2.10-2.01 (m, 1H), 2.00-1.91 (m, 2H), 1.81-1.79 (m, 4H), 1.65-1.57 (m, 6H), 1.17-1.16 (m, 5H), 1.04-0.98 (m, 3H), 0.86-0.76 (m, 6H). m/z: ES⁺ [M+H]⁺=563.30.

### Example 5.62 1-(5-fluoro-2-((5-((1S,5R)-2-((S)-2-methylpyrrolidine-2-carbonyl)-2,6-diazabicyclo[3.2.1]octan-6-yl)-1,2,4-triazin-6-yl)oxy)phenyl)-6-isopropylpyridin-2(1H)-one

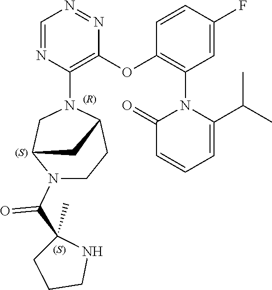

Colourless solid (36 mg, 15% over 3 steps).

¹H NMR (400 MHz, DMSO-d₆, 297.4 K) δ 8.60-8.40 (m, 1H), 7.60-7.45 (m, 1H), 7.18-7.10 (m, 1H), 7.10-6.90 (m, 1H), 6.90-6.70 (m, 1H), 6.60-6.40 (m, 1H), 5.50-5.00 (m, 1H), 5.00-4.70 (m, 1H), 4.50-3.90 (m, 1H), 3.90-3.40 (m, 2H), 3.30-2.90 (m, 2H), 2.90-2.70 (m, 2H), 2.20-2.00 (m, 2H), 2.00-1.60 (m, 7H), 1.60-1.20 (m, 4H), 1.20-1.00 (m, 6H). m/z: ES⁺ [M+H]⁺=548.50.

### Example 5.63 ((S)-2-ethylpyrrolidin-2-yl)((1S,5R)-6-(6-(4-fluoro-2-(3-isopropyl-5-methylisoxazol-4-yl)phenoxy)-1,2,4-triazin-5-yl)-2,6-diazabicyclo[3.2.1]octan-2-yl)methanone

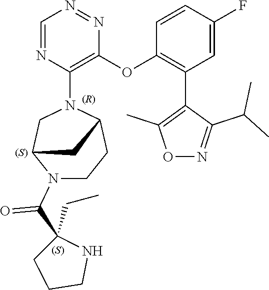

Colourless solid (6 mg, 7% over 3 steps).

¹H NMR (400 MHz, DMSO-d₆, 293.5 K) δ 8.65-8.40 (m, 1H), 8.31 (s, 1H), 7.75-7.45 (m, 1H), 7.43-7.20 (m, 2H), 5.40-5.10 (m, 1H), 4.95-4.70 (m, 1H), 4.40-4.20 (m, 1H), 4.15-3.95 (m, 1H), 3.85-3.70 (m, 1H), 3.65-3.50 (m, 1H), 3.30-3.10 (m, 1H), 2.90-2.70 (m, 3H), 2.57-2.55 (m, 1H), 2.35-2.05 (m, 4H), 1.95-1.75 (m, 2H), 1.70-1.35 (m, 7H), 1.20-0.90 (m, 5H), 0.85-0.65 (m, 3H). m/z: ES⁺ [M+H]⁺=550.25.

### Example 5.64 ((1S,5R)-6-(6-(2-(1,4-diisopropyl-1H-imidazol-5-yl)-4-fluorophenoxy)-1,2,4-triazin-5-yl)-2,6-diazabicyclo[3.2.1]octan-2-yl)((S)-2-methylpyrrolidin-2-yl)methanone

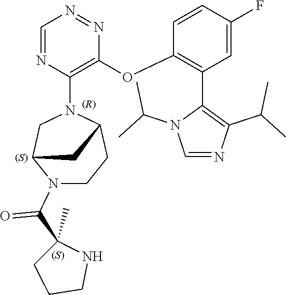

Off-white solid (54 mg, 20% over 3 steps).

¹H NMR (400 MHz, DMSO-d₆, 294.4 K) δ 8.53-8.47 (m, 1H), 7.75-7.31 (m, 4H), 5.51-4.90 (m, 1H), 4.82-4.69 (m, 1H), 4.55-4.00 (m, 1H), 3.95-3.47 (m, 4H), 3.35-2.83 (m, 2H), 2.75-2.63 (m, 2H), 2.52-2.15 (m, 1H), 2.08-1.81 (m, 2H), 1.75-1.45 (m, 5H), 1.43-1.27 (m, 5H), 1.17-0.74 (m, 10H). m/z: ES⁺ [M+H]⁺=563.30.

### Example 5.65 ((1S,5R)-6-(6-(2-(2-ethyl-5-isopropyl-1H-imidazol-1-yl)-4-fluorophenoxy)-1,2,4-triazin-5-yl)-2,6-diazabicyclo[3.2.1]octan-2-yl)((S)-2-methylpyrrolidin-2-yl)methanone

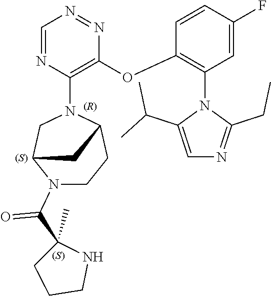

Off-white solid (88 mg, 36% over 3 steps).

¹H NMR (400 MHz, DMSO-d₆, 293.2 K) δ 8.57-8.45 (m, 1H), 8.24 (s, 2H), 7.76-7.51 (m, 3H), 6.62-6.56 (m, 1H), 5.35-5.10 (m, 1H), 4.73-4.64 (m, 1H), 4.40-4.02 (m, 1H), 3.84-3.15 (m, 2H), 3.10-2.88 (m, 3H), 2.75-2.08 (m, 5H), 1.95-1.70 (m, 5H), 1.66-1.30 (m, 5H), 1.10-0.79 (m, 9H). m/z: ES⁺ [M+H]⁺=549.25.

### Example 5.66 ((1S,5R)-6-(6-(4-fluoro-2-(4-isopropyl-1-methyl-1H-1,2,3-triazol-5-yl)phenoxy)-1,2,4-triazin-5-yl)-2,6-diazabicyclo[3.2.1]octan-2-yl)((S)-2-methylpyrrolidin-2-yl)methanone

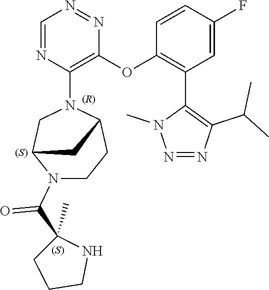

Colourless solid (12 mg, 5% over 3 steps).

¹H NMR (400 MHz, DMSO-d₆, 295.5 K) δ 10.12-9.95 (m, 1H), 9.12-8.99 (m, 1H), 8.95-8.75 (m, 1H), 7.92-7.70 (m, 1H), 7.67-7.50 (m, 2H), 5.32-4.78 (m, 2H), 4.30-3.86 (m, 2H), 3.84-3.73 (m, 3H), 3.60-3.30 (m, 1H), 3.24-3.15 (m, 1H), 3.13-2.80 (m, 1H), 2.80-2.70 (m, 1H), 2.38-2.13 (m, 2H), 2.13-1.73 (m, 6H), 1.70-1.40 (m, 4H), 1.22-0.89 (m, 6H). m/z: ES⁺ [M+H]⁺=536.30.

### Example 5.67 ((1S,5R)-6-(6-(4-fluoro-2-(4-isopropyl-1,3-dimethyl-1H-pyrazol-5-yl)phenoxy)-1,2,4-triazin-5-yl)-2,6-diazabicyclo[3.2.1]octan-2-yl)((S)-2-methylpyrrolidin-2-yl)methanone

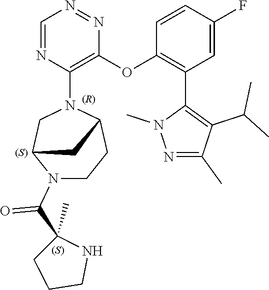

Colourless solid (102 mg, 24% over 3 steps).

¹H NMR (400 MHz, DMSO-d₆, 293.7 K) δ 8.55-8.45 (m, 1H), 7.61-7.27 (m, 3H), 5.63-5.16 (m, 1H), 4.95-4.74 (m, 1H), 4.67-4.26 (m, 1H), 4.02-3.34 (m, 5H), 3.21-2.71 (m, 3H), 2.65-2.03 (m, 6H), 1.91-1.85 (m, 2H), 1.80-1.42 (m, 5H), 1.59-1.10 (m, 3H), 1.05-0.72 (m, 6H). m/z: ES⁺ [M+H]⁺=549.20.

### Example 5.68 ((1S,5R)-6-(6-(4-fluoro-2-(1-isopropyl-3,4-dimethyl-1H-pyrazol-5-yl)phenoxy)-1,2,4-triazin-5-yl)-2,6-diazabicyclo[3.2.1]octan-2-yl)((S)-2-methylpyrrolidin-2-yl)methanone

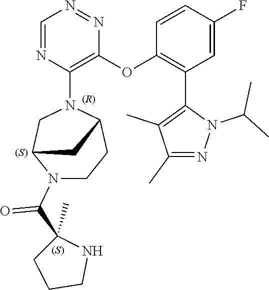

Colourless solid (98 mg, 23% over 3 steps).

¹H NMR (400 MHz, DMSO-d₆, 294.6 K) δ 8.53-8.47 (m, 1H), 8.27 (s, 1H), 7.58-7.56 (m, 1H), 7.52-7.45 (m, 1H), 7.40-7.30 (m, 1H), 5.25-5.16 (m, 1H), 4.74-4.53 (m, 1H), 4.24-3.85 (m, 3H), 3.81-3.78 (m, 1H), 3.01-2.81 (m, 3H), 2.35-2.25 (m, 1H), 2.16-2.00 (m, 3H), 1.90-1.66 (m, 8H), 1.62-1.56 (m, 2H), 1.41 (s, 3H), 1.34-1.23 (m, 2H), 1.13-1.07 (m, 2H), 0.99-0.90 (m, 3H). m/z: ES⁺ [M+H]⁺=549.25.

### Example 5.69 ((1S,5R)-6-(6-(4-fluoro-2-(3-(isopropyl(methyl)amino)oxetan-3-yl)phenoxy)-1,2,4-triazin-5-yl)-2,6-diazabicyclo[3.2.1]octan-2-yl)((S)-2-methylpyrrolidin-2-yl)methanone

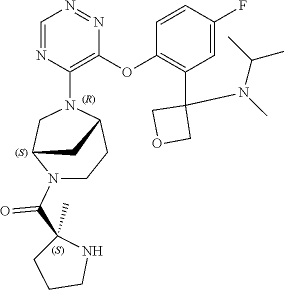

Brown solid, TFA salt (46 mg, 7% over 3 steps)

¹H NMR (400 MHz, DMSO-d₆, 293.4 K) δ 8.72-8.46 (m, 1H), 7.49-7.19 (m, 3H), 5.79-5.14 (m, 1H), 5.14-4.63 (m, 5H), 4.63-4.20 (m, 1H), 4.18-3.90 (m, 1H), 3.90-3.62 (m, 1H), 3.31-3.05 (m, 1H), 3.08-2.89 (m, 1H), 2.89-2.72 (m, 2H), 2.69-2.58 (m, 1H), 2.42-2.16 (m, 4H), 2.14-1.79 (m, 3H), 1.79-1.46 (m, 4H), 1.46-1.17 (m, 3H), 0.89-0.65 (m, 6H). m/z: ES⁺ [M+H]⁺=510.25.

### Example 5.70 N-ethyl-N-(2-(5-fluoro-2-((5-((1S,5R)-2-((S)-2-methylpyrrolidine-2-carbonyl)-2,6-diazabicyclo[3.2.1]octan-6-yl)-1,2,4-triazin-6-yl)oxy)phenyl)propan-2-yl)acetamide

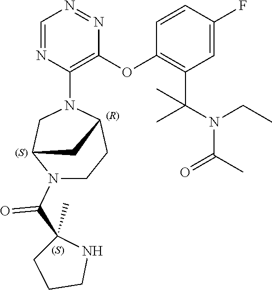

Colourless solid, TFA salt (7 mg, 5% over 3 steps)

¹H NMR (400 MHz, DMSO-d₆, 296.0 K) δ 8.56-8.43 (m, 1H), 8.31 (s, 1H), 7.42-6.71 (m, 3H), 5.38-4.12 (m, 4H), 3.91-2.82 (m, 7H), 2.38-1.72 (m, 9H), 1.72-1.46 (m, 11H), 1.22-0.90 (m, 3H). m/z: ES⁺ [M+H]⁺=540.30.

### Example 5.71 ((1S,5R)-6-(6-(2-(3-(dimethylamino)oxetan-3-yl)-4-fluorophenoxy)-1,2,4-triazin-5-yl)-2,6-diazabicyclo[3.2.1]octan-2-yl)((S)-2-methylpyrrolidin-2-yl)methanone

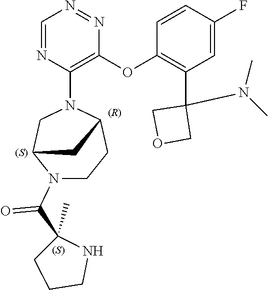

Brown solid, TFA salt (46 mg, 15% over 3 steps)

¹H NMR (400 MHz, DMSO-d₆, 293.4 K) δ 9.65-9.12 (m, 1H), 8.96-8.79 (m, 1H), 8.79-8.60 (m, 1H), 7.63-7.32 (m, 3H), 5.21-4.77 (m, 8H), 4.46-4.13 (m, 1H), 3.99-3.71 (m, 2H), 3.60-3.39 (m, 1H), 3.31-3.01 (m, 3H), 2.85-2.57 (m, 6H), 2.38-1.70 (m, 8H), 1.70-1.49 (m, 3H). m/z: ES⁺ [M+H]⁺=512.20.

### Example 5.72 ((1S,5R)-6-(6-(4-fluoro-2-(5-isopropyl-4-methyl-1H-imidazol-1-yl)phenoxy)-1,2,4-triazin-5-yl)-2,6-diazabicyclo[3.2.1]octan-2-yl)((S)-2-methylpyrrolidin-2-yl)methanone

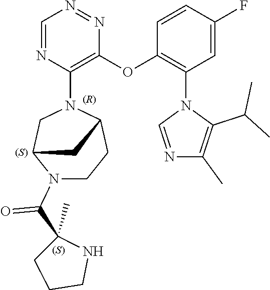

Colourless solid (16 mg, % over 3 steps).

¹H NMR (400 MHz, DMSO-d₆, 294.2 K) δ 8.53-8.49 (m, 1H), 7.67-7.64 (m, 1H), 7.55-7.53 (m, 2H), 7.51-7.30 (m, 1H), 5.50-5.05 (m, 1H), 4.78-4.65 (m, 1H), 4.50-4.30 (m, 1H), 3.99-3.77 (m, 1H), 3.59-3.56 (m, 1H), 3.29-3.25 (m, 1H), 3.00-2.91 (m, 1H), 2.89-2.88 (m, 1H), 2.71-2.62 (m, 1H), 2.35-2.24 (m, 1H), 2.12-2.07 (m, 3H), 1.91-1.90 (m, 2H), 1.70-1.66 (m, 5H), 1.37-1.32 (m, 4H), 1.23-0.99 (m, 6H). m/z. ES⁺ [M+H]⁺=535.30.

### Example 5.73 ((1S,5R)-6-(6-(4-fluoro-2-(2-isopropyl-4-methyl-1H-imidazol-1-yl)phenoxy)-1,2,4-triazin-5-yl)-2,6-diazabicyclo[3.2.1]octan-2-yl)((S)-2-methylpyrrolidin-2-yl)methanone

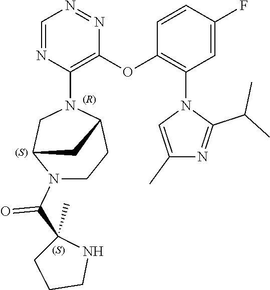

Colourless solid (42 mg, 22% over 3 steps).

¹H NMR (400 MHz, DMSO-d₆, 294.6K) δ 15.29 (br s, 1H), 9.80-9.29 (m, 1H), 8.97-8.80 (m, 1H), 8.80-8.58 (m, 1H), 8.00-7.77 (m, 2H), 7.77-7.68 (m, 1H), 7.68-7.30 (m, 1H), 5.35-4.95 (m, 1H), 4.94-4.78 (m, 1H), 4.20-4.08 (m, 2H), 3.99-3.86 (m, 1H), 3.80-3.71 (m, 1H), 3.51-3.28 (m, 1H), 3.27-2.89 (m, 4H), 2.30-2.25 (m, 3H), 2.25-2.16 (m, 1H), 2.10-1.70 (m, 6H), 1.69-1.60 (m, 3H), 1.51-1.28 (m, 3H), 1.28-0.96 (m, 3H). m/z: ES⁺ [M+H]⁺=535.25.

### Example 5.74 4-(5-fluoro-2-((5-((1S,5R)-2-((S)-2-methylpyrrolidine-2-carbonyl)-2,6-diazabicyclo[3.2.1]octan-6-yl)-1,2,4-triazin-6-yl)oxy)phenyl)-3-isopropyl-1,5-dimethylpyridin-2(1H)-one

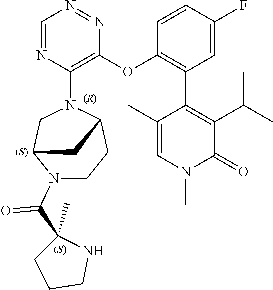

Colourless solid, formic acid salt (17 mg, 16% over 3 steps).

¹H NMR (400 MHz, DMSO-d₆, 296.2 K) δ 8.59-8.42 (m, 1H), 8.21 (s, 1H), 7.67-7.42 (m, 2H), 7.41-7.27 (m, 1H), 7.26-6.98 (m, 1H), 5.38-5.02 (m, 1H), 4.99-4.61 (m, 1H), 4.59-4.05 (m, 2H), 3.95-3.45 (m, 4H), 3.44-3.15 (m, 5H), 3.02-2.88 (m, 2H), 2.83-2.69 (m, 1H), 2.29-2.12 (m, 1H), 1.99-1.89 (m, 2H), 1.88-1.47 (m, 8H), 1.40-1.29 (m, 3H), 1.19-1.01 (m, 3H), 0.98-0.72 (m, 2H). m/z: ES⁺ [M+H]⁺=576.30.

### Examples 5.75 and 5.76: atropisomers of 4-(5-fluoro-2-((5-((1S,5R)-2-((S)-2-methylpyrrolidine-2-carbonyl)-2,6-diazabicyclo[3.2.1]octan-6-yl)-1,2,4-triazin-6-yl)oxy)phenyl)-5-isopropyl-1,3-dimethylpyridin-2(1H)-one

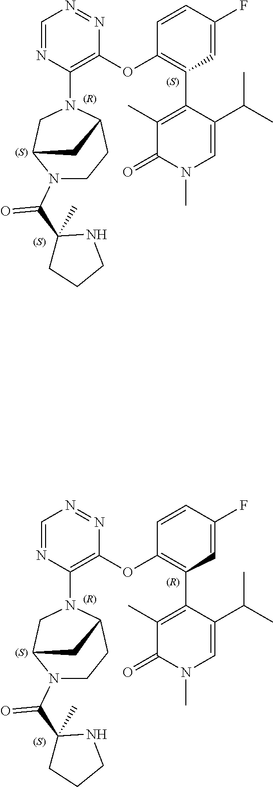

Separation by chiral HPLC gave two single stable atropisomers:

Peak #1: Rt=12.38 min, colourless solid (87 mg, 1.8%).

¹H NMR (400 MHz, DMSO-d₆, 295.0 K) δ 8.74-8.39 (m, 1H), 7.63-7.52 (m, 1H), 7.47-7.32 (m, 2H), 7.28-7.15 (m, 1H), 5.52-4.92 (m, 1H), 4.85-4.46 (m, 1H), 4.32-4.04 (m, 1H), 3.99-3.53 (m, 3H), 3.50-3.23 (m, 4H), 3.01-3.72 (m, 2H), 2.70-2.55 (m, 1H), 2.33-2.18 (m, 2H), 1.97-1.45 (m, 9H), 1.38-1.2 (m, 3H), 1.02-0.49 (m, 6H). m/z: ES⁺ [M+H]⁺=576.35.

Peak #2: Rt=16.72 min, colourless solid (107 mg, 2%).

¹H NMR (400 MHz, DMSO-d₆, 295.4 K) δ 8.74-8.39 (m, 1H), 8.32-8.07 (m, 1H), 7.63-7.52 (m, 1H), 7.47-7.32 (m, 1H), 7.28-7.15 (m, 1H), 5.52-4.92 (m, 1H), 4.85-4.46 (m, 1H), 4.32-4.04 (m, 1H), 3.99-3.53 (m, 3H), 3.50-3.23 (m, 4H), 3.01-2.72 (m, 2H), 2.70-2.55 (m, 1H), 2.33-2.18 (m, 2H), 1.97-1.45 (m, 9H), 1.38-1.2 (m, 3H), 1.02-0.49 (m, 6H). m/z: ES⁺ [M+H]⁺=576.30

### Example 5.77 6-(5-fluoro-2-((5-((1S,5R)-2-((S)-2-methylpyrrolidine-2-carbonyl)-2,6-diazabicyclo[3.2.1]octan-6-yl)-1,2,4-triazin-6-yl)oxy)phenyl)-5-isopropyl-1-methylpyrimidin-2(1H)-one

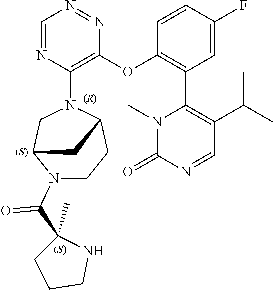

Colourless solid (7 mg, 2% over 3 steps).

¹H NMR (400 MHz, DMSO-d₆, 296.0 K) δ 8.66-8.63 (m, 1H), 8.54-8.50 (m, 1H), 7.65-7.53 (m, 3H), 5.76-5.18 (m, 1H), 4.95-4.85 (m, 1H), 4.73-4.22 (m, 1H), 3.96-3.75 (m, 2H), 3.12-2.83 (m, 6H), 2.75-2.64 (m, 1H), 2.32-2.07 (m, 2H), 2.05-1.85 (m, 2H), 1.75-1.62 (m, 5H), 1.26 (s, 3H), 1.10-0.95 (m, 3H), 0.95-0.75 (m, 3H). m/z: ES⁺ [M+H]⁺=563.15.

### Example 5.78 5-(5-fluoro-2-((5-((1S,5R)-2-((S)-2-methylpyrrolidine-2-carbonyl)-2,6-diazabicyclo[3.2.1]octan-6-yl)-1,2,4-triazin-6-yl)oxy)phenyl)-6-isopropyl-1-methylpyrazin-2(1H)-one

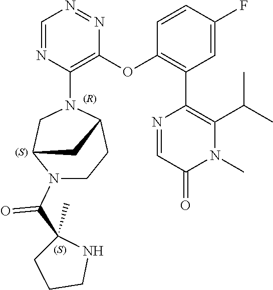

Colourless solid (7 mg, 7% over 3 steps).

¹H NMR (400 MHz, DMSO-d₆, 295.7 K) δ 8.57-8.37 (m, 1H), 8.26 (s, 1H), 8.15-7.85 (m, 1H), 7.58-7.50 (m, 1H), 7.49-7.39 (m, 1H), 7.38-7.29 (m, 1H), 5.57-4.95 (m, 1H), 4.86-4.52 (m, 1H), 4.50-4.01 (m, 1H), 3.98-3.88 (m, 3H), 3.87-3.69 (m, 1H), 3.52-3.48 (m, 1H), 2.98-2.73 (m, 3H), 2.71-2.60 (m, 1H), 2.28-2.15 (m, 1H), 1.93-1.35 (m, 7H), 1.33-1.16 (m, 4H), 1.14-0.83 (m, 6H). m/z: ES⁺ [M+H]⁺=563.30.

### Examples 5.79 and 5.80 atropisomers of ((1S,5R)-6-(6-(4-fluoro-2-(5-isopropyl-2,4-dimethyl-1H-imidazol-1-yl)phenoxy)-1,2,4-triazin-5-yl)-2,6-diazabicyclo[3.2.1]octan-2-yl)((S)-2-methylpyrrolidin-2-yl)methanone

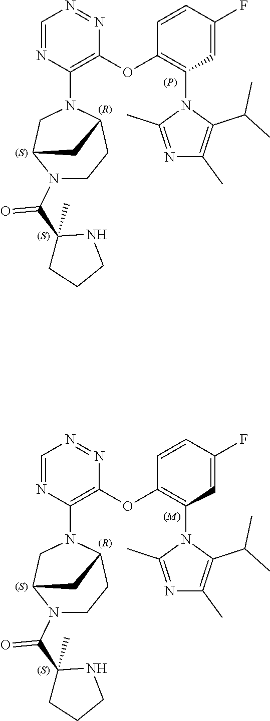

Separation by chiral HPLC gave two single stable atropisomers:

Peak #1: Rt=11.25 min, colourless solid (116 mg, 3%).

¹H NMR (400 MHz, DMSO-d₆, 294.0 K) δ 8.62-8.34 (m, 1H), 7.82-7.65 (m, 1H), 7.65-7.41 (m, 2H), 5.85-4.92 (m, 1H), 4.80-4.56 (m, 1H), 4.56-4.04 (m, 1H), 3.95-3.66 (m, 1H), 3.61-3.40 (m, 1H), 3.21-2.98 (m, 1H), 2.98-2.75 (m, 1H), 2.70-2.60 (m, 1H), 2.40-2.19 (m, 2H), 2.12-2.00 (m, 2H), 2.00-1.97 (m, 6H), 1.79-1.34 (m, 6H), 1.34-1.10 (m, 4H), 1.05-0.95 (m, 2H), 0.90-0.80 (m, 1H), 0.80-0.51 (m, 2H). m/z: ES⁺ [M+H]⁺=549.35.

Peak #2: Rt=14.33 min, colourless solid (198 mg, 5%).

¹H NMR (400 MHz, DMSO-d₆, 300.5 K) δ 8.59-8.34 (m, 1H), 7.78-7.60 (m, 1H), 7.60-7.39 (m, 2H), 5.55-4.87 (m, 1H), 4.87-4.61 (m, 1H), 4.61-4.08 (m, 1H), 3.90-3.40 (m, 3H), 2.95-2.78 (m, 1H), 2.72-2.60 (m, 1H), 2.46-2.40 (m, 1H), 2.30-2.20 (m, 1H), 2.12-2.00 (m, 3H), 2.00-1.85 (m, 5H), 1.76-1.50 (m, 5H), 1.38-1.19 (m, 4H), 1.05-0.88 (m, 5H), 0.75-0.54 (m, 1H). m/z: ES⁺ [M+H]⁺=549.30

### Examples 5.81 and 5.82 atropisomers of ((1S,5R)-6-(6-(3,4-difluoro-2-(2-isopropyl-4,5-dimethyl-1H-imidazol-1-yl)phenoxy)-1,2,4-triazin-5-yl)-2,6-diazabicyclo[3.2.1]octan-2-yl)((S)-2-methylpyrrolidin-2-yl)methanone

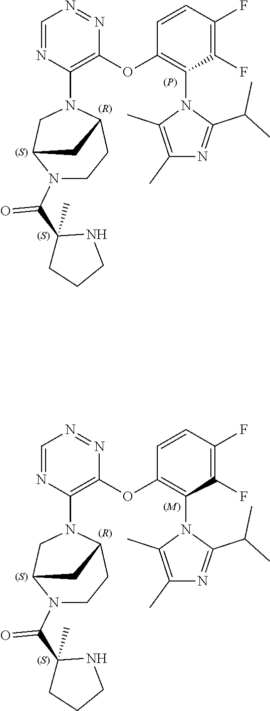

Separation by chiral HPLC gave two single stable atropisomers:

Peak #1: Rt=20.22 min, off-white solid (17 mg, 3%).

¹H NMR (400 MHz, DMSO-d₆, 295.4 K) δ 8.62-8.41 (m, 1H), 8.25 (s, 0.61H), 7.95-7.70 (m, 1H), 7.69-7.40 (m, 1H), 5.50-5.10 (m, 1H), 4.83-4.69 (m, 1H), 4.35-4.20 (m, 1H), 3.80-3.75 (m, 1H), 3.65-3.58 (m, 1H), 3.52-3.40 (m, 1H), 3.38-3.18 (m, 1H), 3.05-2.83 (m, 2H), 2.80-2.66 (m, 1H), 2.61-2.52 (m, 1H), 2.38-2.18 (m, 1H), 2.02-1.92 (m, 4H), 1.85-1.80 (m, 2H), 1.78-1.55 (m, 6H), 1.36 (s, 3H), 1.00-0.89 (m, 5H). m/z: ES⁺ [M+H]⁺=567.30.

Peak #2: Rt=28.37 min, off-white solid (17 mg, 3%).

¹H NMR (400 MHz, DMSO-d₆, 296.3 K) δ 8.78-8.39 (m, 1H), 8.00-7.70 (m, 1H), 7.69-7.50 (m, 1H), 5.72-4.91 (m, 1H), 4.90-4.45 (m, 1H), 4.44-4.10 (m, 1H), 4.00-3.70 (m, 1H), 3.67-3.51 (m, 1H), 3.10-2.80 (m, 1H), 2.78-2.58 (m, 1H), 2.30-2.19 (m, 1H), 2.18-1.96 (m, 2H), 1.95-1.90 (m, 2H), 1.89-1.79 (m, 5H), 1.78-1.51 (m, 6H), 1.50-1.08 (m, 4H), 1.05-0.91 (m, 3H), 0.90-0.68 (m, 3H). m/z: ES⁺ [M+H]⁺=567.30

### Example 5.83 ((1S,5R)-6-(6-(2-(2,4-diisopropyl-1H-imidazol-1-yl)-4-fluorophenoxy)-1,2,4-triazin-5-yl)-2,6-diazabicyclo[3.2.1]octan-2-yl)((S)-2-methylpyrrolidin-2-yl)methanone

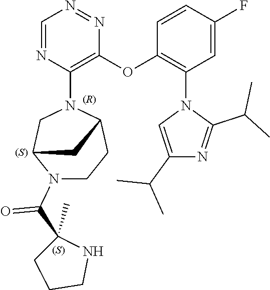

Colourless solid (39 mg, 7% over 3 steps).

¹H NMR (400 MHz, DMSO-d₆, 296.1 K) δ 8.55-8.43 (m, 1H), 8.26 (s, 2H), 7.67-7.46 (m, 3H), 6.60-6.40 (m, 1H), 5.22-4.90 (m, 1H), 4.83-4.64 (m, 1H), 4.30-3.89 (m, 1H), 3.81-3.61 (m, 1H), 3.18-2.99 (m, 1H), 2.94-2.80 (m, 1H), 2.77-2.56 (m, 3H), 2.47-2.20 (m, 2H), 2.00-1.55 (m, 7H), 1.53-1.34 (m, 4H), 1.17-0.88 (m, 12H). m/z: ES⁺ [M+H]⁺=563.25.

### Example 5.84 ((1S,5R)-6-(6-(2-((RS)-1-(dimethylamino)-2,2,2-trifluoroethyl)-4-fluorophenoxy)-1,2,4-triazin-5-yl)-2,6-diazabicyclo[3.2.1]octan-2-yl)((S)-2-methylpyrrolidin-2-yl)methanone

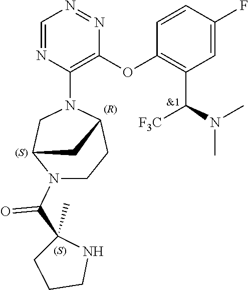

Brown solid, HCl salt (73 mg, 47% over 3 steps)

¹H NMR (400 MHz, DMSO-d₆, 300.6 K) δ 9.91-9.54 (m, 1H), 9.12-8.98 (m, 1H), 8.97-8.70 (m, 1H), 7.81-7.39 (m, 3H), 5.41-4.80 (m, 3H), 4.51-4.21 (m, 1H), 4.18-3.60 (m, 2H), 3.60-3.35 (m, 1H), 3.34-3.00 (m, 3H), 2.60-2.55 (m, 1H), 2.48-2.42 (m, 2H), 2.35-2.25 (m, 2H), 2.25-1.75 (m, 8H), 1.75-1.46 (m, 3H). m/z: ES⁺ [M+H]⁺=538.30.

### Example 5.85 ((1S,5R)-6-(6-(2-(2-(tert-butyl)-4,5-dimethyl-1H-imidazol-1-yl)-4-fluorophenoxy)-1,2,4-triazin-5-yl)-2,6-diazabicyclo[3.2.1]octan-2-yl)((S)-2-methylpyrrolidin-2-yl)methanone

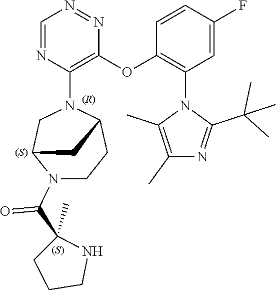

Colourless solid, formic acid salt (74 mg, 29% over 3 steps).

¹H NMR (400 MHz, DMSO-d₆, 295.3 K) δ 8.65-8.43 (m, 1H), 8.29 (s, 1H), 7.98-6.98 (m, 3H), 5.35-4.96 (m, 1H), 4.95-4.06 (m, 2H), 3.93-3.81 (m, 1H), 3.71-3.56 (m, 1H), 3.41-2.77 (m, 4H), 2.31-1.80 (m, 9H), 1.79-1.38 (m, 8H), 1.18-0.79 (m, 9H).

m/z: ES⁺ [M+H]⁺=563.25.

### Example 5.86 ((1S,5R)-6-(6-(2-(4-ethyl-2-isopropyl-5-methyl-1H-imidazol-1-yl)-4-fluorophenoxy)-1,2,4-triazin-5-yl)-2,6-diazabicyclo[3.2.1]octan-2-yl)((S)-2-methylpyrrolidin-2-yl)methanone

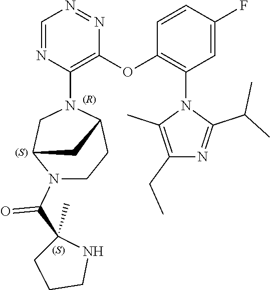

Colourless solid, formic acid salt (30 mg, 19% over 3 steps).

¹H NMR (400 MHz, DMSO-d₆, 294.3 K) δ 8.60-8.45 (m, 1H), 8.35-8.15 (m, 1H), 7.80-7.10 (m, 3H), 5.40-5.10 (m, 1H), 4.80-4.70 (m, 1H), 4.40-4.30 (m, 1H), 4.20-4.10 (m, 1H), 3.80-3.75 (m, 1H), 3.65-3.55 (m, 1H), 3.45-3.35 (m, 1H), 3.20-2.90 (m, 2H). 2.85-2.70 (m, 1H). 2.60-2.50 (m, 1H). 2.40-2.10 (m, 3H). 2.10-1.95 (m, 2H). 2.00-1.45 (m, 8H). 1.45-1.25 (m, 3H). 1.10-0.75 (m, 8H). m/z: ES⁺ [M+H]⁺=563.30.

### Examples 5.87 and 5.88 atropisomers of ((1S,5R)-6-(6-(2-(4-ethyl-5-isopropyl-2-methyl-1H-imidazol-1-yl)-4-fluorophenoxy)-1,2,4-triazin-5-yl)-2,6-diazabicyclo[3.2.1]octan-2-yl)((S)-2-methylpyrrolidin-2-yl)methanone

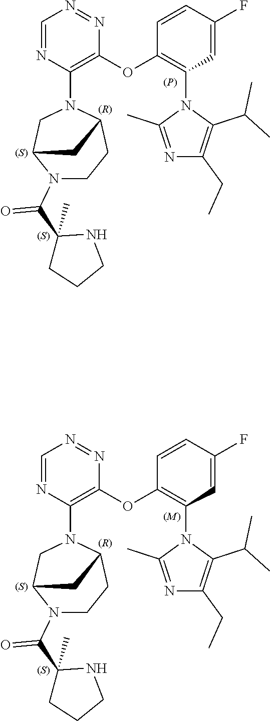

Separation by chiral HPLC gave two single stable atropisomers:

Peak #1: Rt=24.46 min, colourless solid (3 mg, 3%).

¹H NMR (400 MHz, DMSO-d₆, 295.6K) δ 8.55-8.52 (m, 1H), 7.74-7.54 (m, 3H), 5.60-5.16 (m, 1H), 4.79-4.34 (m, 2H), 3.77-3.36 (m, 2H), 2.90-2.64 (m, 3H), 2.46-2.08 (m, 4H), 2.00-1.91 (m, 4H), 1.90-1.78 (m, 1H), 1.78-1.51 (m, 5H), 1.32-1.24 (m, 4H), 1.10-0.95 (m, 6H), 0.88-0.72 (m, 3H). m/z: ES⁺ [M+H]⁺=577.35.

Peak #2: Rt=28.66 min, colourless solid (3 mg, 3%).

¹H NMR (400 MHz, DMSO-d₆, 295.7 K) δ 8.55-8.51 (m, 1H), 7.71-7.68 (m, 1H), 7.64-7.53 (m, 2H), 5.50-5.16 (m, 1H), 4.78-4.25 (m, 2H), 3.73-3.57 (m, 2H), 3.11-2.97 (m, 1H), 2.90-2.80 (m, 1H), 2.67-2.63 (m, 1H), 2.50-2.44 (m, 2H), 2.33-2.24 (m, 1H), 2.10-2.02 (m, 3H), 1.89-1.70 (m, 5H), 1.28-1.05 (m, 4H), 1.05-0.96 (m, 9H), 0.91-0.78 (m, 3H). m/z: ES⁺ [M+H]⁺=577.30.

### Example 5.89 ((1S,5R)-6-(6-(4-fluoro-2-(2-isopropyl-5,6-dihydrocyclopenta[d]imidazol-1(4H)-yl)phenoxy)-1,2,4-triazin-5-yl)-2,6-diazabicyclo[3.2.1]octan-2-yl)((S)-2-methylpyrrolidin-2-yl)methanone

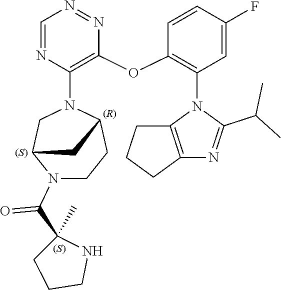

Colourless solid, formic acid salt (48 mg, 20% over 3 steps).

¹H NMR (400 MHz, DMSO-d₆, 297.3 K) δ 8.56-8.41 (m, 1H), 8.27 (s, 1H), 7.72-7.57 (m, 2H), 7.54-7.46 (m, 1H), 5.35-5.20 (m, 1H), 4.82-4.69 (m, 2H), 4.35-4.19 (m, 1H), 3.94-3.30 (m, 3H), 3.09-2.93 (m, 1H), 2.91-2.87 (m, 1H), 2.69-2.58 (m, 1H), 2.48-2.37 (m, 3H), 2.37-2.12 (m, 4H), 1.98-1.51 (m, 8H), 1.51-1.42 (m, 3H), 1.21-1.06 (m, 1H), 1.07-1.00 (m, 1H), 1.00-0.81 (m, 3H). m/z: ES⁺ [M+H]⁺=561.30.

### Example 5.90 ((1S,5R)-6-(6-(4-fluoro-2-(2-methyl-6,7-dihydro-5H-pyrrolo[1,2-a]imidazol-3-yl)phenoxy)-1,2,4-triazin-5-yl)-2,6-diazabicyclo[3.2.1]octan-2-yl)((S)-2-methylpyrrolidin-2-yl)methanone

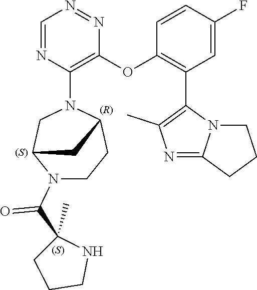

Brown solid, HCl salt (92 mg, 39% over 3 steps)

¹H NMR (400 MHz, DMSO-d₆, 296.7 K) δ 15.06 (s, 1H), 9.94 (s, 1H), 9.02-8.98 (m, 1H), 8.89 (s, 1H), 7.77-7.67 (m, 1H), 7.62-7.57 (m, 2H), 5.27-5.15 (m, 1H), 4.99-4.88 (m, 1H), 4.34-4.25 (m, 1H), 4.09-4.02 (m, 1H), 3.91-3.87 (m, 1H), 3.82-3.87 (m, 1H), 3.70-3.71 (m, 1H), 3.66-3.67 (m, 1H), 3.41-3.49 (m, 1H), 3.20-3.29 (m, 1H), 3.09-3.12 (m, 3H), 2.64-2.65 (m, 2H), 2.15-2.18 (m, 3H), 1.97-2.07 (m, 6H), 1.65 (s, 3H). m/z: ES⁺ [M+H]⁺=533.30.

### Example 5.91 ((1S,5R)-6-(6-(4-fluoro-2-(2-isopropyl-6,7-dihydro-5H-pyrrolo[1,2-a]imidazol-3-yl)phenoxy)-1,2,4-triazin-5-yl)-2,6-diazabicyclo[3.2.1]octan-2-yl)((S)-2-methylpyrrolidin-2-yl)methanone

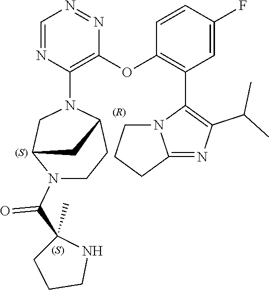

Colourless solid (39 mg, 39% over 3 steps)

¹H NMR (400 MHz, DMSO-d₆, 296.8 K) δ 8.67-8.31 (m, 1H), 7.65-7.42 (m, 1H), 7.42-7.31 (m, 1H), 7.31-7.21 (m, 1H), 5.82-4.98 (m, 1H), 4.98-4.65 (m, 1H), 4.65-4.07 (m, 1H), 4.06-3.43 (m, 4H), 3.12-2.68 (m, 3H), 2.71-2.58 (m, 4H), 2.47-2.36 (m, 1H), 2.30-2.13 (m, 1H), 2.09-1.80 (m, 2H), 1.80-1.38 (m, 6H), 1.31-1.22 (m, 3H), 1.03-0.85 (m, 6H). m/z: ES⁺ [M+H]⁺=561.30.

### Examples 5.92 and 5.93 atropisomers of ((1S,5R)-6-(6-(2-(5-ethyl-2-isopropyl-4-methyl-1H-imidazol-1-yl)-4-fluorophenoxy)-1,2,4-triazin-5-yl)-2,6-diazabicyclo[3.2.1]octan-2-yl)((S)-2-methylpyrrolidin-2-yl)methanone

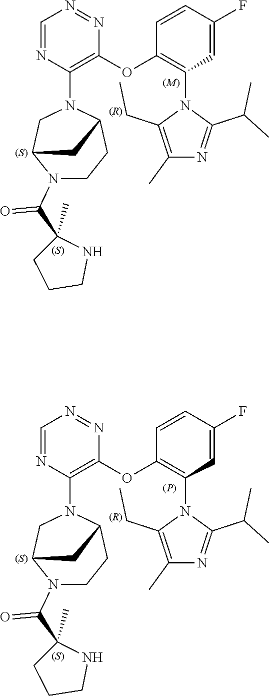

Separation by chiral HPLC gave two single stable atropisomers:

Peak #1: Rt=19.90 min, colourless solid (9 mg, 3%).

¹H NMR (400 MHz, DMSO-d₆, 294.2 K) δ 8.72-8.41 (m, 1H), 7.59-7.32 (m, 3H), 5.75-4.90 (m, 1H), 4.80-4.50 (m, 1H), 4.45-4.04 (m, 1H), 3.95-3.50 (m, 2H), 3.15-2.98 (m, 1H), 2.88-2.56 (m, 2H), 2.32-2.15 (m, 2H), 2.10-1.82 (m, 5H), 1.80-1.52 (m, 6H), 1.50-1.42 (m, 3H), 1.30-0.90 (m, 4H), 0.90-0.72 (m, 6H). m/z: ES⁺ [M+H]⁺=563.35.

Peak #2: Rt=29.29 min, colourless solid (13 mg, 4%).

¹H NMR (400 MHz, DMSO-d₆, 294.2 K) δ 8.72-8.41 (m, 1H), 7.89-7.52 (m, 3H), 5.75-4.90 (m, 1H), 4.80-4.04 (m, 2H), 3.95-3.50 (m, 2H), 3.15-2.80 (m, 2H), 2.72-2.56 (m, 3H), 2.32-2.15 (m, 3H), 2.10-1.82 (m, 5H), 1.80-1.52 (m, 5H), 1.50-1.22 (m, 3H), 1.10-0.90 (m, 3H), 0.90-0.72 (m, 6H). m/z: ES⁺ [M+H]⁺=563.35.

### Examples 5.94 and 5.95 atropisomers of ((1S,5R)-6-(6-(2-(2,5-diisopropyl-4-methyl-1H-imidazol-1-yl)-4-fluorophenoxy)-1,2,4-triazin-5-yl)-2,6-diazabicyclo[3.2.1]octan-2-yl)((S)-2-methylpyrrolidin-2-yl)methanonee

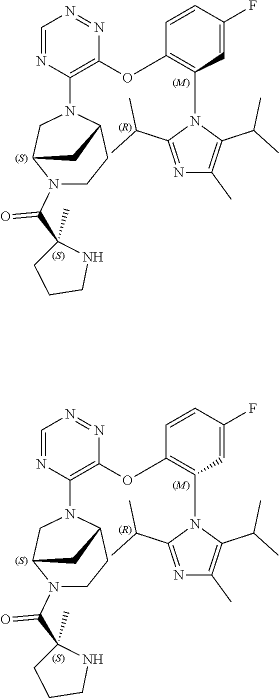

Separation by chiral HPLC gave two single stable atropisomers:

Peak #1: Rt=13.0 min, colourless solid (15 mg, 2%).

¹H NMR (400 MHz, DMSO-d₆, 294.1 K) δ 8.55-8.52 (m, 1H), 7.74-7.54 (m, 3H), 5.60-5.16 (m, 1H), 4.79-4.34 (m, 2H), 3.77-3.36 (m, 2H), 2.90-2.64 (m, 3H), 2.46-2.08 (m, 3H), 2.00-1.91 (m, 4H), 1.78-1.51 (m, 6H), 1.32-1.24 (m, 4H), 1.10-1.06 (m, 3H), 0.99-0.95 (m, 5H), 0.88-0.83 (m, 2H), 0.81-0.72 (m, 2H). m/z: ES⁺ [M+H]⁺=577.35.

Peak #2: Rt=15.64 min, colourless solid (19 mg, 3%).

¹H NMR (400 MHz, DMSO-d₆, 294.8 K) δ 8.55-8.51 (m, 1H), 7.71-7.68 (m, 1H), 7.64-7.61 (m, 1H), 7.57-7.53 (m, 1H), 5.50-5.16 (m, 1H), 4.78-4.52 (m, 2H), 3.73-3.57 (m, 2H), 3.11-2.97 (m, 1H), 2.90-2.80 (m, 1H), 2.67-2.63 (m, 1H), 2.50-2.44 (m, 2H), 2.33-2.24 (m, 1H), 2.10-2.02 (m, 3H), 1.89-1.83 (m, 2H), 1.83-1.70 (m, 5H), 1.28-1.04 (m, 4H), 0.99-0.96 (m, 9H), 0.91-0.78 (m, 3H). m/z: ES⁺ [M+H]⁺=577.30.

### Examples 5.96 and 5.97 atropisomers of 5-(5-fluoro-2-((5-((1S,5R)-2-((S)-2-methylpyrrolidine-2-carbonyl)-2,6-diazabicyclo[3.2.1]octan-6-yl)-1,2,4-triazin-6-yl)oxy)phenyl)-4-isopropyl-1,6-dimethylpyridin-2(1H)-one

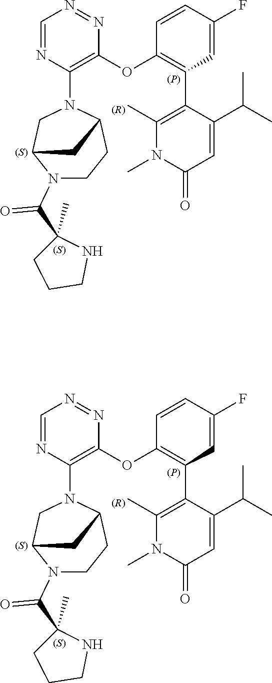

Separation by chiral HPLC gave two single stable atropisomers:

Peak #1: Rt=4.28 min, colourless solid (87 mg, 10%).

¹H NMR (400 MHz, DMSO-d₆, 297.4 K) 68.52-8.42 (m, 1H), 8.25 (s, 1H), 7.62-7.51 (m, 1H), 7.49-7.32 (m, 2H), 6.20 (s, 1H), 5.35-5.02 (m, 1H), 4.72-4.50 (m, 1H), 4.30-4.15 (m, 1H), 3.92-3.75 (m, 2H), 3.59-3.52 (m, 1H), 3.52-3.35 (m, 2H), 3.05-2.55 (m, 4H), 2.35-2.15 (m, 2H), 2.14-1.85 (m, 4H), 1.80-1.42 (m, 6H), 1.45-1.20 (m, 3H), 1.10-0.55 (m, 6H). m/z: ES⁺ [M+H]⁺=576.30.

Peak #2: Rt=8.77 min, colourless solid (90 mg, 10%).

¹H NMR (400 MHz, DMSO-d₆, 297.4 K) δ 8.52-7.42 (m, 1H), 8.25 (s, 1H), 7.62-7.51 (m, 1H), 7.49-7.32 (m, 2H), 6.35-6.10 (m, 1H), 5.35-5.02 (m, 1H), 4.72-4.50 (m, 1H), 4.30-4.15 (m, 1H), 3.92-3.75 (m, 2H), 3.59-3.50 (m, 1H), 3.38 (s, 3H), 3.05-2.85 (m, 2H), 2.75-2.68 (m, 1H), 2.35-2.15 (m, 2H), 2.14-1.82 (m, 5H), 1.80-1.42 (m, 5H), 1.45-1.20 (m, 3H), 0.95-0.55 (m, 6H). m/z: ES⁺ [M+H]⁺=576.35.

### Example 5.98 1-(5-fluoro-2-((5-((1S,5R)-2-((S)-2-methylpyrrolidine-2-carbonyl)-2,6-diazabicyclo[3.2.1]octan-6-yl)-1,2,4-triazin-6-yl)oxy)phenyl)-2,6-dimethylpyridin-4(1H)-one

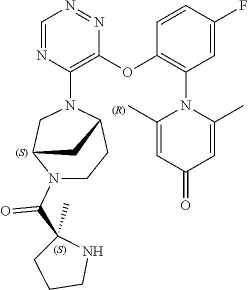

Colourless solid, formic acid salt (17 mg, 23% over 3 steps).

¹H NMR (400 MHz, DMSO-d₆, 297.0 K) δ 8.68-8.47 (m, 1H), 8.21 (br s, 2.5H), 7.88-7.61 (m, 2H), 7.60-7.40 (m, 1H), 6.11-5.82 (m, 2H), 5.50-5.12 (m, 1H), 4.72 (s, 1H), 4.40-4.25 (m, 1H), 4.01-3.91 (m, 1H), 3.84-3.75 (m, 1H), 3.62-3.65 (m, 1H), 2.92-2.86 (m, 1H), 2.75-2.71 (m, 1H), 2.29-2.12 (m, 1H), 2.02-1.83 (m, 8H), 1.82-1.55 (m, 6H), 1.25 (s, 3H). m/z: ES⁺ [M+H]⁺=534.25.

### Example 5.99 1-(5-fluoro-2-((5-((1S,5R)-2-((S)-2-methylpyrrolidine-2-carbonyl)-2,6-diazabicyclo[3.2.1]octan-6-yl)-1,2,4-triazin-6-yl)oxy)phenyl)-2,6-diisopropylpyridin-4(1H)-one

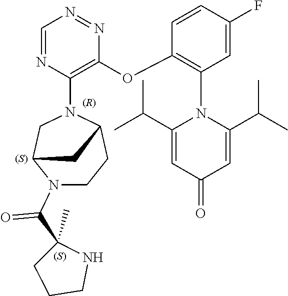

Colourless solid, formic acid salt (10 mg, 10% over 3 steps).

¹H NMR (400 MHz, DMSO-d₆, 296.6K) δ 10.09-9.63 (1H), 8.91-8.75 (m, 2H), 8.25-8.10 (m, 1H), 8.09-7.89 (m, 1H), 7.88-7.61 (m, 1H), 7.59-7.34 (m, 2H), 5.25-4.89 (m, 1H), 4.88-4.69 (m, 1H), 4.31-4.18 (m, 1H), 3.91-3.85 (m, 2H), 3.81-3.79 (m, 1H), 3.25-3.17 (m, 1H), 3.08-3.05 (m, 1H), 2.78-2.61 (m, 2H), 2.38-2.11 (m, 2H), 2.10-1.63 (m, 5H), 1.62-1.41 (m, 4H), 1.31-1.09 (m, 9H), 1.08-0.92 (m, 3H).

m/z: ES⁺ [M+H]⁺=590.30.

### Example 5.100 ((1S,5R)-6-(6-(4-fluoro-2-(4-isopropyl-1,2-dimethyl-1H-imidazol-5-yl)phenoxy)-1,2,4-triazin-5-yl)-2,6-diazabicyclo[3.2.1]octan-2-yl)((S)-2-methylpyrrolidin-2-yl)methanone

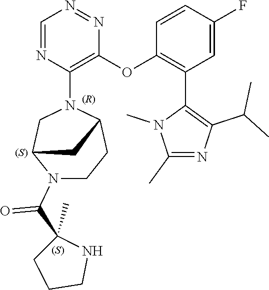

Colourless solid (67 mg, 25% over 3 steps).

¹H NMR (400 MHz, DMSO-d₆, 299.2 K) 69.01-8.86 (m, 1H), 7.65-7.51 (m, 2H), 7.51-7.32 (m, 2H), 5.50-4.80 (m, 2H), 4.51-4.20 (m, 1H), 4.20-3.78 (m, 2H), 3.63-3.55 (m, 1H), 3.50-3.36 (m, 3H), 3.31-3.18 (m, 1H), 3.18-3.05 (m, 1H), 2.88-2.61 (m, 1H), 2.61-2.58 (m, 4H), 2.40-2.15 (m, 2H), 2.15-1.75 (m, 6H), 1.75-1.50 (m, 3H), 1.31-1.20 (m, 2H), 1.20-1.13 (m, 2H), 1.13-0.99 (m, 2H). m/z: ES⁺ [M+H]⁺=549.30.

### Example 5.101 ((1S,5R)-6-(6-(4-fluoro-2-(1-isopropyl-2,5-dimethyl-1H-imidazol-4-yl)phenoxy)-1,2,4-triazin-5-yl)-2,6-diazabicyclo[3.2.1]octan-2-yl)((S)-2-methylpyrrolidin-2-yl)methanone

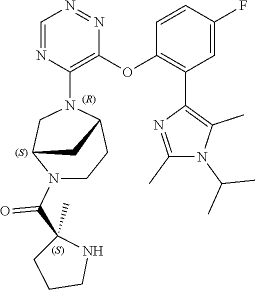

Colourless solid (39 mg, 6% over 3 steps).

¹H NMR (400 MHz, DMSO-d₆, 298.9 K) δ 8.50-8.29 (m, 1H), 7.52-7.30 (m, 1H), 7.29-7.18 (m, 1H), 7.19-7.10 (m, 1H), 5.42-5.31 (m, 1H), 5.16-4.78 (m, 1H), 4.56-4.29 (m, 2H), 4.10-3.80 (m, 1H), 3.88-3.69 (m, 1H), 3.60-3.46 (m, 1H), 2.98-2.81 (m, 2H), 2.66-2.59 (m, 1H), 2.25-2.21 (m, 1H), 2.18 (s, 3H), 2.06 (s, 3H), 2.00-1.90 (m, 1H), 1.78-1.49 (m, 6H), 1.35-1.20 (m, 9H). m/z: ES⁺ [M+H]⁺=549.30.

### Examples 5.102 and 5.103 atropisomers of 6-(5-fluoro-2-((5-((1S,5R)-2-((S)-2-methylpyrrolidine-2-carbonyl)-2,6-diazabicyclo[3.2.1]octan-6-yl)-1,2,4-triazin-6-yl)oxy)phenyl)-5-isopropyl-1,4-dimethylpyridin-2(1H)-one

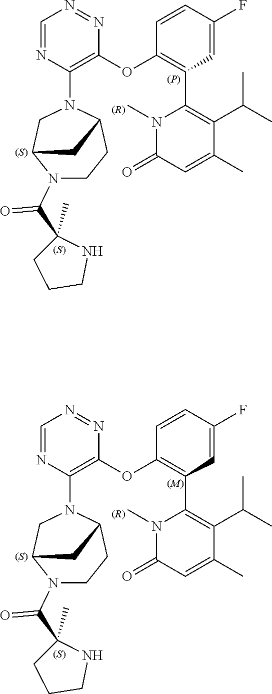

Separation by chiral HPLC gave two single stable atropisomers:

Peak #1: Rt=13.25 min, colourless solid (47 mg, 5%).

¹H NMR (400 MHz, DMSO-d₆, 298.7 K) δ 8.60-8.50 (m, 1H), 7.65-7.51 (m, 1H), 7.48-7.46 (m, 2H), 6.34-6.21 (m, 1H), 5.25-5.11 (m, 1H), 4.77-4.42 (m, 1H), 4.28-4.01 (m, 2H), 3.95-3.79 (m, 1H), 3.71-3.60 (m, 1H), 3.50-3.46 (m, 1H), 3.01-2.97 (m, 6H), 2.89-2.66 (m, 1H), 2.23 (s, 3H), 2.08-1.53 (m, 7H), 1.47 (s, 3H), 1.04 (d, J=7.2 Hz, 3H), 0.85-0.75 (m, 3H). m/z: ES⁺ [M+H]⁺=576.30.

Peak #2: Rt=17.33 min, colourless solid (27 mg, 3%).

¹H NMR (400 MHz, DMSO-d₆, 298.1 K) δ 8.60-8.50 (m, 1H), 8.30 (s, 2H), 7.64-7.62 (m, 1H), 7.52-7.50 (m, 1H), 7.48-7.43 (m, 1H), 6.33-6.26 (m, 1H), 5.16-4.73 (m, 2H), 4.20-4.00 (m, 2H), 3.77-3.67 (m, 3H), 3.32-3.29 (m, 2H), 3.13-2.89 (m, 4H), 2.70-2.67 (m, 1H), 2.18-2.16 (m, 4H), 2.07-1.97 (m, 2H), 1.92-1.76 (m, 2H), 1.88-1.46 (m, 4H), 1.32-1.15 (m, 1H), 1.03 (s, 3H), 0.91-0.70 (m, 3H). m/z: ES⁺ [M+H]⁺=576.35.

### Example 5.104 5-(5-fluoro-2-((5-((1S,5R)-2-((S)-2-methylpyrrolidine-2-carbonyl)-2,6-diazabicyclo[3.2.1]octan-6-yl)-1,2,4-triazin-6-yl)oxy)phenyl)-6-isopropyl-1-methylpyridin-2(1H)-one

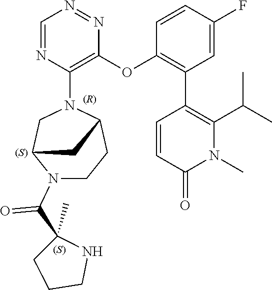

Colourless solid (15 mg, 4% over 3 steps).

¹H NMR (400 MHz, DMSO-d₆, 296.2 K) δ 9.30 (br s, 1H), 8.88 (br s, 1H), 8.65 (s, 1H), 7.65-7.60 (m, 1H), 7.55-7.50 (m, 1H), 7.45-7.40 (m, 1H), 7.39-7.30 (m, 1H), 6.40-6.25 (m, 1H), 5.30-5.20 (m, 1H), 5.10-4.70 (m, 2H), 4.51-4.15 (m, 1H), 4.10-3.65 (m, 2H), 3.60-3.50 (m, 3H), 3.45-2.80 (m, 3H), 2.50-2.15 (m, 2H), 2.10-1.50 (m, 9H), 1.45-1.15 (m, 3H), 1.12-0.60 (m, 3H). m/z: ES⁺ [M+H]⁺=562.25.

### Example 5.105 ((1S,5R)-6-(6-(4-fluoro-2-(5-isopropyl-6-methylpyridazin-4-yl)phenoxy)-1,2,4-triazin-5-yl)-2,6-diazabicyclo[3.2.1]octan-2-yl)((S)-2-methylpyrrolidin-2-yl)methanone

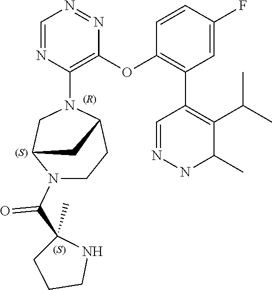

Light-yellow solid (40 mg, 30% over 3 steps).

¹H NMR (400 MHz, DMSO-d₆, 297.4 K) δ 10.14-9.98 (m, 1H), 9.28-9.02 (m, 2H), 8.88-8.86 (m, 1H), 7.80-7.78 (m, 1H), 7.77-7.62 (m, 1H), 7.47-7.45 (m, 1H), 5.25-5.18 (m, 1H), 4.99-4.94 (m, 1H), 4.27-3.89 (m, 3H), 3.55-3.15 (m, 4H), 3.07-2.95 (m, 3H), 2.84-2.81 (m, 1H), 2.29-2.18 (m, 2H), 2.08-1.77 (m, 6H), 1.66 (s, 3H), 1.28-1.12 (m, 6H). m/z: ES⁺ [M+H]⁺=547.30.

### Examples 5.106 and 5.107 atropisomers of ((1S,5R)-6-(6-(4-fluoro-2-(5-isopropyl-3-methylpyridazin-4-yl)phenoxy)-1,2,4-triazin-5-yl)-2,6-diazabicyclo[3.2.1]octan-2-yl)((S)-2-methylpyrrolidin-2-yl)methanone

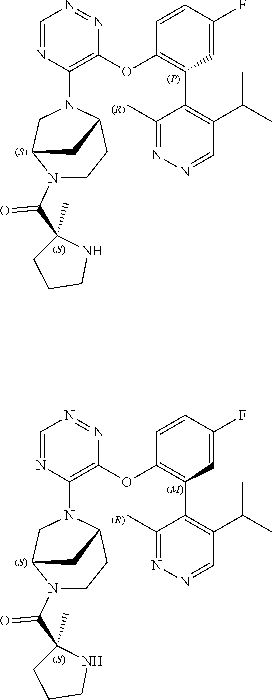

Separation by chiral HPLC gave two single stable atropisomers:

Peak #1: Rt=11.77 min, colourless solid (18 mg, 6%).

¹H NMR (400 MHz, DMSO-d₆, 296.7 K) δ 9.16 (s, 1H), 8.59-8.32 (m, 1H), 7.87-7.62 (m, 1H), 7.47-7.32 (m, 2H), 5.59-5.20 (m, 1H), 4.83-4.63 (m, 1H), 4.64-4.21 (m, 2H), 3.87-3.72 (m, 1H), 3.68-3.52 (m, 1H), 3.06-2.85 (m, 1H), 2.76-2.53 (m, 2H), 2.36-2.21 (m, 3H), 2.19-2.07 (m, 1H), 1.97-1.92 (m, 1H), 1.84-1.52 (m, 6H), 1.43-1.21 (m, 3H), 1.19-0.96 (m, 7H). m/z: ES⁺ [M+H]⁺=547.25.

Peak #2: Rt=18.31 min, colourless solid (15 mg, 5%).

¹H NMR (400 MHz, DMSO-d₆, 296.7 K) δ 9.15 (s, 1H), 8.59-8.32 (m, 1H), 7.77-7.62 (m, 1H), 7.47-7.32 (m, 2H), 5.59-5.20 (m, 1H), 4.83-4.73 (m, 1H), 4.64-4.11 (m, 2H), 3.87-3.68 (m, 1H), 3.62-3.52 (m, 1H), 2.96-2.85 (m, 2H), 2.76-2.53 (m, 2H), 2.36-2.21 (m, 3H), 2.19-2.07 (m, 1H), 1.97-1.92 (m, 1H), 1.84-1.52 (m, 6H), 1.33-1.21 (m, 3H), 1.19-1.03 (m, 3H), 1.00-0.87 (m, 3H). m/z: ES⁺ [M+H]⁺=547.25.

### Example 5.108 ((1S,5R)-6-(6-(4-fluoro-2-(1-isopropyl-2-methyl-1H-imidazol-5-yl)phenoxy)-1,2,4-triazin-5-yl)-2,6-diazabicyclo[3.2.1]octan-2-yl)((S)-2-methylpyrrolidin-2-yl)methanone

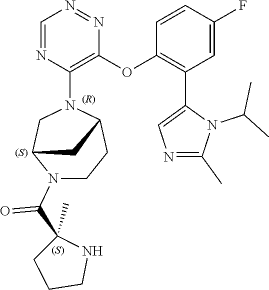

Colourless solid (63 mg, 29% over 3 steps).

¹H NMR (400 MHz, DMSO-d₆, 295.9 K) δ 8.50-8.30 (m, 1H), 7.56-7.50 (m, 1H), 7.59-7.38 (m, 1H), 7.36-7.30 (m, 1H), 6.50 (s, 1H), 5.60-5.20 (m, 1H), 4.89-4.80 (m, 1H), 4.31-4.10 (m, 2H), 3.93-3.72 (m, 1H), 3.70-3.58 (m, 1H), 3.16-3.04 (m, 1H), 2.99-2.89 (m, 1H), 2.67-2.60 (m, 1H), 2.33-2.23 (m, 3H), 2.30-2.420 (m, 1H), 1.96-1.84 (m, 2H), 1.77-1.37 (m, 5H), 1.38-1.24 (m, 10H). m/z: ES⁺ [M+H]⁺=535.30.

### Example 5.109 ((1S,5R)-6-(6-(4-fluoro-2-(1-isopropyl-2,4-dimethyl-1H-imidazol-5-yl)phenoxy)-1,2,4-triazin-5-yl)-2,6-diazabicyclo[3.2.1]octan-2-yl)((S)-2-methylpyrrolidin-2-yl)methanone

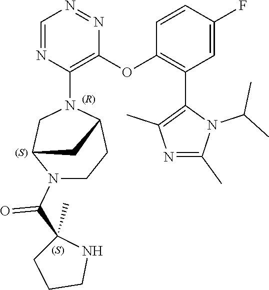

Colourless solid, formic acid salt (73 mg, 14% over 3 steps).

¹H NMR (400 MHz, DMSO-d₆, 294.2 K) δ 8.60-8.40 (m, 1H), 8.25 (s, 2H), 7.69-7.52 (m, 1H), 7.46-7.34 (m, 1H), 7.32-7.24 (m, 1H), 5.41-5.30 (m, 1H), 5.10-5.00 (m, 1H), 4.89-4.70 (m, 1H), 4.21-3.70 (m, 4H), 3.33-2.72 (m, 3H), 2.30-2.18 (m, 4H), 2.06-1.79 (m, 9H), 1.43-1.23 (m, 3H), 1.33-1.03 (m, 6H), 1.00-0.90 (m, 1H). m/z: ES⁺ [M+H]⁺=549.25.

### Example 5.110 ((1S,5R)-6-(6-(2-(2-cyclopropyl-5-(trifluoromethyl)-1H-imidazol-1-yl)-4-fluorophenoxy)-1,2,4-triazin-5-yl)-2,6-diazabicyclo[3.2.1]octan-2-yl)((S)-2-ethylpyrrolidin-2-yl)methanone

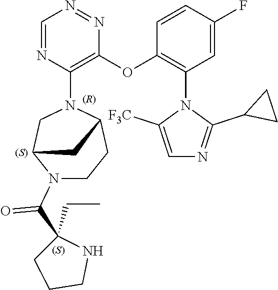

Off-white solid, formic acid salt (11 mg, 20% over 3 steps).

¹H NMR (400 MHz, DMSO-d₆, 295.6K) δ 8.72-8.49 (m, 1H), 8.23 (s, 1H), 7.89-7.72 (m, 2H), 7.70-7.25 (m, 1H), 5.42-5.24 (m, 2H), 4.97-4.64 (m, 2H), 4.34-4.12 (m, 1H), 3.86-3.57 (m, 2H), 2.94-2.63 (m, 2H), 2.44-2.12 (m, 1H), 2.04-1.32 (m, 11H), 1.01-0.77 (m, 5H), 0.67-0.35 (m, 2H). m/z: ES⁺ [M+H]⁺=601.25.

### Example 5.111 ((1S,5R)-6-(6-(4-fluoro-2-(((S)-tetrahydrofuran-3-yl)oxy)phenoxy)-1,2,4-triazin-5-yl)-2,6-diazabicyclo[3.2.1]octan-2-yl)((S)-2-methylpyrrolidin-2-yl)methanone

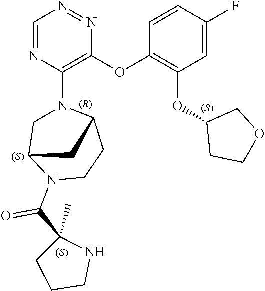

Off-white solid, formic acid salt (46 mg, 29% over 3 steps).

¹H NMR (400 MHz, DMSO-d₆, 295.5 K) δ 8.81-8.47 (m, 1H), 8.27 (s, 1H), 7.48-7.25 (m, 1H), 7.25-7.11 (m, 1H), 6.97-6.73 (m, 1H), 5.41-5.30 (m, 1H), 5.16-4.75 (m, 2H), 4.48-4.13 (m, 1H), 4.13-3.85 (m, 1H), 3.85-3.62 (m, 3H), 3.53-3.38 (m, 2H), 3.30-3.10 (m, 2H), 3.10-2.92 (m, 1H), 2.92-2.73 (m, 1H), 2.35-2.21 (m, 1H), 2.21-1.96 (m, 3H), 1.96-1.63 (m, 6H), 1.55-1.31 (m, 3H). m/z: ES⁺ [M+H]⁺=499.20.

### Example 5.112 ((1S,5R)-6-(6-(4-fluoro-2-(((R)-tetrahydrofuran-3-yl)oxy)phenoxy)-1,2,4-triazin-5-yl)-2,6-diazabicyclo[3.2.1]octan-2-yl)((S)-2-methylpyrrolidin-2-yl)methanone

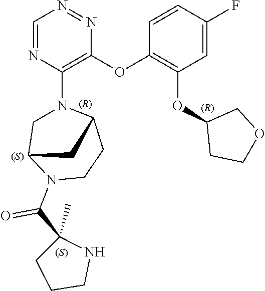

Off-white solid, HCl salt (34 mg, 16% over 3 steps).

¹H NMR (400 MHz, DMSO-d₆, 296.3 K) δ 9.99 (br s, 1H), 9.23-9.00 (m, 1H), 8.98-8.69 (m, 1H), 7.62-7.35 (m, 1H), 7.34-7.08 (m, 1H), 7.02-6.74 (m, 1H), 5.44-5.28 (m, 1H), 5.21-4.83 (m, 2H), 4.50-4.26 (m, 1H), 4.19-3.93 (m, 1H), 3.90-3.74 (m, 2H), 3.71-3.33 (m, 4H), 3.30-3.15 (m, 1H), 3.14-2.88 (m, 1H), 2.34-2.26 (m, 1H), 2.23-1.74 (m, 9H), 1.72-1.52 (m, 3H). m/z: ES⁺ [M+H]⁺=499.15.

### Example 5.113 5-(5-fluoro-2-((5-((1S,5R)-2-((S)-2-methylpyrrolidine-2-carbonyl)-2,6-diazabicyclo[3.2.1]octan-6-yl)-1,2,4-triazin-6-yl)oxy)phenyl)-6-isopropyl-4-methylpyridin-2(1H)-one

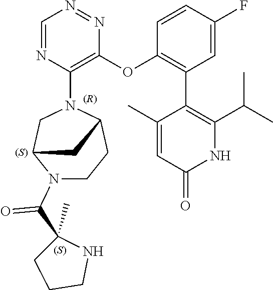

Off-white solid, formic acid salt (24 mg, 14% over 3 steps).

¹H NMR (400 MHz, DMSO-d₆, 295.6 K) δ 8.72-8.49 (m, 1H), 8.30-8.21 (m, 1H), 7.89-7.72 (m, 2H), 7.70-7.25 (m, 1H), 6.20-6.00 (m, 1H), 5.42-5.24 (m, 1H), 4.97-4.64 (m, 2H), 4.34-4.12 (m, 2H), 3.86-3.57 (m, 2H), 2.94-2.63 (m, 2H), 2.44-2.12 (m, 1H), 2.04-1.32 (m, 11H), 1.01-0.77 (m, 6H), 0.67-0.35 (m, 3H). m/z: ES⁺ [M+H]⁺=562.30.

### Example 5.114 ((1S,5R)-6-(6-(4-fluoro-2-(1-isopropyl-4,5-dimethyl-1H-imidazol-2-yl)phenoxy)-1,2,4-triazin-5-yl)-2,6-diazabicyclo[3.2.1]octan-2-yl)((S)-2-methylpyrrolidin-2-yl)methanone

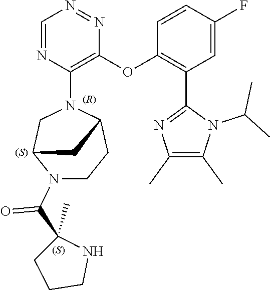

Colourless solid (22 mg, 12% over 3 steps).

¹H NMR (400 MHz, DMSO-d₆, 297.5 K) δ 8.30-8.51 (m, 1H), 7.60-7.48 (m, 1H), 7.47-7.37 (m, 1H), 7.32-7.19 (m, 1H), 5.62-5.02 (m, 1H), 5.02-4.66 (m, 1H), 4.66-3.89 (m, 2H), 3.89-3.42 (m, 1H), 3.12-2.70 (m, 3H), 2.70-2.58 (m, 2H), 2.30-2.07 (m, 4H), 1.98-1.85 (m, 4H), 1.85-1.45 (m, 6H), 1.35-1.08 (m, 9H). m/z: ES⁺ [M+H]⁺=549.30.

### Example 5.115 ((1S,5R)-6-(6-(4-fluoro-2-(5-isopropyl-1-methyl-1H-imidazol-4-yl)phenoxy)-1,2,4-triazin-5-yl)-2,6-diazabicyclo[3.2.1]octan-2-yl)((S)-2-methylpyrrolidin-2-yl)methanone

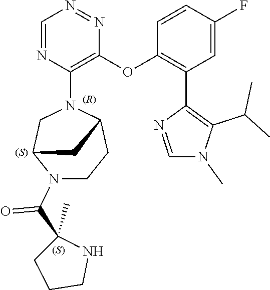

Light-yellow solid, formic acid salt (30 mg, 27% over 3 steps).

¹H NMR (400 MHz, DMSO-d₆, 295.3 K) 68.52-8.05 (m, 2H), 7.62-7.51 (m, 2H), 7.49-7.32 (m, 1H), 7.25-7.10 (m, 1H), 5.42-5.02 (m, 1H), 4.92-4.50 (m, 1H), 4.12-3.98 (m, 1H), 3.92-3.70 (m, 1H), 3.65-3.55 (m, 4H), 3.42-2.56 (m, 5H), 2.32-2.15 (m, 1H), 2.10-1.52 (m, 7H), 1.50-1.42 (m, 3H), 1.22-0.95 (m, 6H). m/z: ES⁺ [M+H]⁺=535.30.

### Example 5.116 N-(5-fluoro-2-((5-((1S,5R)-2-((S)-2-methylpyrrolidine-2-carbonyl)-2,6-diazabicyclo[3.2.1]octan-6-yl)-1,2,4-triazin-6-yl)oxy)benzyl)-N-isopropylacetamide

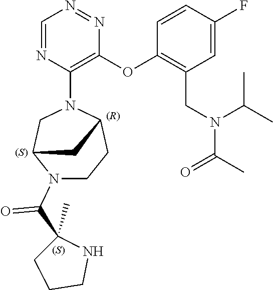

Off-white solid, formic acid salt (107 mg, 29% over 3 steps)

¹H NMR (400 MHz, DMSO-d₆, 295.6 K) δ 8.52-7.40 (m, 1H), 8.29 (s, 1H), 7.49-7.10 (m, 2H), 7.00-6.88 (m, 1H), 5.35-5.25 (m, 1H), 5.20-4.82 (m, 1H), 4.78-4.02 (m, 5H), 3.98-3.62 (m, 1H), 3.60-2.92 (m, 2H), 2.85-2.70 (m, 1H), 2.40-2.25 (m, 1H), 2.12-2.09 (m, 3H), 2.07-1.98 (m, 2H), 1.90-1.52 (m, 6H), 1.50-1.42 (m, 3H), 1.15-0.88 (m, 6H). m/z: ES⁺ [M+H]⁺=526.20.

### Example 5.117 N-ethyl-N-(5-fluoro-2-((5-((1S,5R)-2-((S)-2-methylpyrrolidine-2-carbonyl)-2,6-diazabicyclo[3.2.1]octan-6-yl)-1,2,4-triazin-6-yl)oxy)benzyl)acetamide

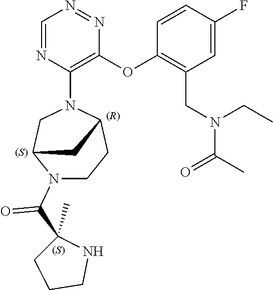

Off-white solid, formic acid salt (30 mg, 20% over 3 steps)

¹H NMR (400 MHz, DMSO-d₆, 296.9 K) δ 8.60-8.40 (m, 1H), 8.25 (s, 1H), 7.42-6.99 (m, 3H), 5.38-5.20 (m, 1H), 5.12-4.90 (m, 1H), 4.56-4.40 (m, 2H), 4.35-4.20 (m, 2H), 4.15-3.60 (m, 2H), 3.31-3.09 (m, 2H), 3.05-2.96 (m, 1H), 2.85-2.70 (m, 1H), 2.35-2.20 (m, 1H), 2.15-2.00 (m, 2H), 2.00-1.95 (m, 2H), 1.90-1.84 (m, 2H), 1.81-1.64 (m, 5H), 1.39 (s, 3H), 1.10-1.01 (m, 2H), 0.98-0.87 (m, 1H). m/z: ES⁺ [M+H]⁺=512.25.

### Example 5.118 1-(5-fluoro-2-((5-((1S,5R)-2-((S)-2-methylpyrrolidine-2-carbonyl)-2,6-diazabicyclo[3.2.1]octan-6-yl)-1,2,4-triazin-6-yl)oxy)benzyl)pyrrolidin-2-one

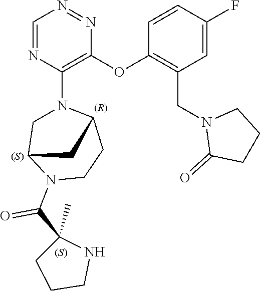

Colourless solid (32 mg, 14% over 3 steps)

¹H NMR (400 MHz, DMSO-d₆, 297.1 K) δ 8.50-8.40 (m, 1H), 7.48-7.10 (m, 3H), 5.68-4.62 (m, 2H), 4.62-4.02 (m, 4H), 3.95-3.33 (m, 2H), 3.21-3.05 (m, 2H), 3.00-2.72 (m, 2H), 2.71-2.59 (m, 1H), 2.32-1.87 (m, 5H), 1.86-1.50 (m, 7H), 1.49-1.11 (m, 3H). m/z: ES⁺ [M+H]⁺=510.25.

### Example 5.119 (R)-1-(5-fluoro-2-((5-((1S,5R)-2-((S)-2-methylpyrrolidine-2-carbonyl)-2,6-diazabicyclo[3.2.1]octan-6-yl)-1,2,4-triazin-6-yl)oxy)benzyl)-5-methylpyrrolidin-2-one

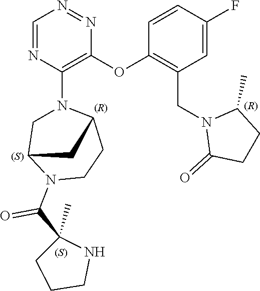

Colourless solid (25 mg, 26% over 3 steps)

¹H NMR (400 MHz, DMSO-d₆, 295.9 K) δ 8.66-8.26 (m, 1H), 7.55-7.02 (m, 3H), 5.61-5.20 (m, 1H), 4.98-4.67 (m, 2H), 4.61-4.05 (m, 2H), 4.01-3.58 (m, 2H), 3.45-3.33 (m, 1H), 3.21-2.93 (m, 1H), 2.92-2.78 (m, 1H), 2.74-2.58 (m, 1H), 2.31-2.11 (m, 2H), 2.10-1.83 (m, 4H), 1.83-1.49 (m, 6H), 1.45-1.34 (m, 1H), 1.31-1.20 (m, 3H), 1.10-0.94 (m, 3H). m/z: ES⁺ [M+H]⁺=524.25.

### Example 5.120 ((1S,5R)-6-(6-(4-fluoro-2-(5-isopropyl-1,2-dimethyl-1H-imidazol-4-yl)phenoxy)-1,2,4-triazin-5-yl)-2,6-diazabicyclo[3.2.1]octan-2-yl)((S)-2-methylpyrrolidin-2-yl)methanone

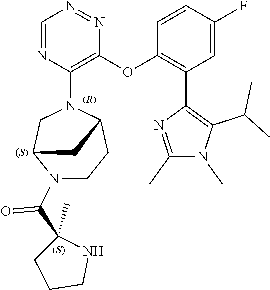

Colourless solid, formic acid salt (2 mg, 1% over 3 steps).

¹H NMR (400 MHz, DMSO-d₆, 296.3 K) 68.49-8.32 (m, 1H), 8.32-8.21 (m, 1.6H), 7.51-7.32 (m, 1H), 7.32-7.21 (m, 1H), 7.13-7.01 (m, 1H), 5.38-4.97 (m, 1H), 4.97-4.78 (m, 1H), 4.39-3.87 (m, 2H), 3.72-3.61 (m, 2H), 3.49-3.41 (m, 3H), 3.33-2.77 (m, 4H), 2.25-2.11 (m, 5H), 2.11-1.83 (m, 4H), 2.83-1.63 (m, 2H), 1.63-1.51 (m, 3H), 1.15-0.96 (m, 6H). m/z: ES⁺ [M+H]⁺=549.20.

### Example 5.121 ((1S,5R)-6-(6-(4-fluoro-2-(1-isopropyl-2-methyl-1H-imidazol-4-yl)phenoxy)-1,2,4-triazin-5-yl)-2,6-diazabicyclo[3.2.1]octan-2-yl)((S)-2-methylpyrrolidin-2-yl)methanone

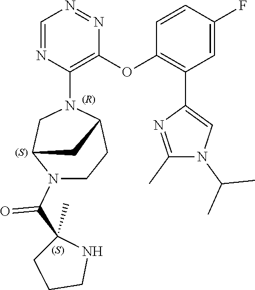

Colourless solid, formic acid salt (96 mg, 34% over 3 steps).

¹H NMR (400 MHz, DMSO-d₆, 296.5 K) δ 8.57-8.43 (m, 1H), 8.24 (s, 2H), 7.81-7.63 (m, 1H), 7.42 (s, 1H), 7.32-7.21 (m, 1H), 7.12-7.06 (m, 1H), 5.33-4.80 (m, 2H), 4.36-3.65 (m, 4H), 3.10-2.72 (m, 3H), 2.45-2.19 (m, 4H), 2.12-1.56 (m, 7H), 1.62-1.28 (m, 3H), 1.27-1.20 (m, 6H). m/z: ES⁺ [M+H]⁺=535.25.

### Example 6.01: N-ethyl-5-fluoro-N-isopropyl-2-((4-((1S,5R)-2-((R)-4-methylmorpholine-3-carbonyl)-2,6-diazabicyclo[3.2.1]octan-6-yl)pyrimidin-5-yl)oxy)benzamide

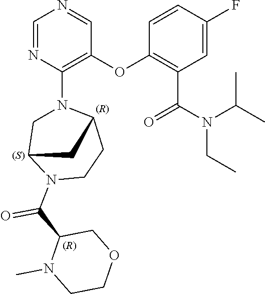

The reductive amination of N-ethyl-5-fluoro-N-isopropyl-2-((4-((1S,5R)-2-((R)-morpholine-3-carbonyl)-2,6-diazabicyclo[3.2.1]octan-6-yl)pyrimidin-5-yl)oxy)benzamide (60 mg, 0.11 mmol) was performed according to general procedure H1. The reaction mixture was concentrated, and the residue was purified using method Prep-HPLC1 (Gradient: 17% B to 40% B in 7 min; RT1(min): 6.63) to afford N-ethyl-5-fluoro-N-isopropyl-2-((4-((1S,5R)-2-((R)-4-methylmorpholine-3-carbonyl)-2,6-diazabicyclo[3.2.1]octan-6-yl)pyrimidin-5-yl)oxy)benzamide (37 mg, 59%) as a colourless solid.

¹H NMR (400 MHz, DMSO-d₆, 300 K) δ 8.51-8.14 (m, 1H), 7.92-7.62 (m, 1H), 7.40-7.10 (m, 2H), 7.09-6.80 (m, 1H), 5.57-5.06 (m, 1H), 4.95-4.55 (m, 1H), 4.54-4.15 (m, 1H), 3.98-3.62 (m, 4H), 3.60-3.41 (m, 3H), 3.32-3.11 (m, 3H), 3.09-3.01 (m, 1H), 2.85-2.68 (m, 1H), 2.22-2.03 (m, 1H), 1.99-1.78 (m, 2H), 1.77-1.42 (m, 2H), 1.25-0.95 (m, 9H). m/z: ES⁺ [M+H]⁺=541.30.

### Example 6.02: 2-((4-((1S,5R)-2-((S)-1,2-dimethylpyrrolidine-2-carbonyl)-2,6-diazabicyclo[3.2.1]octan-6-yl)pyrimidin-5-yl)oxy)-N-ethyl-5-fluoro-N-isopropylbenzamide

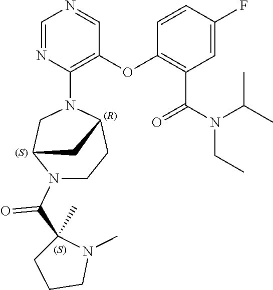

The reductive amination of N-ethyl-5-fluoro-N-isopropyl-2-((4-((1S,5R)-2-((S)-2-methylpyrrolidine-2-carbonyl)-2,6-diazabicyclo[3.2.1]octan-6-yl)pyrimidin-5-yl)oxy)benzamide (120 mg, 0.23 mmol) was performed according to general procedure H1. The reaction mixture was purified using method Prep-HPLC1 (Gradient: 30% B to 55% B in 7 min; RT1(min): 5.5) to afford the title compound (41 mg, 33%) as a colourless solid.

¹H NMR (400 MHz, DMSO-d₆, 300 K) δ 8.38-8.23 (m, 1H), 7.89-7.69 (m, 1H), 7.36-7.13 (m, 2H), 7.05-6.90 (m, 1H), 5.52 (br s, 1H), 4.86-4.02 (m, 2H), 3.90-3.51 (m, 3H), 3.44-3.37 (m, 1H), 3.27-3.10 (m, 2H), 3.05-2.90 (m, 1H), 2.55-2.43 (m, 1H), 2.25-2.05 (m, 4H), 1.95-1.80 (m, 4H), 1.70-1.45 (m, 3H), 1.25-1.17 (m, 1H), 1.14-0.92 (m, 11H). m/z: ES⁺ [M+H]⁺=539.25.

### Example 6.03: ((1S,5R)-6-(5-(2-(2-cyclopropyl-5-methyl-1H-imidazol-1-yl)-4-fluorophenoxy)pyrimidin-4-yl)-2,6-diazabicyclo[3.2.1]octan-2-yl)((S)-1,2-dimethylpyrrolidin-2-yl)methanone

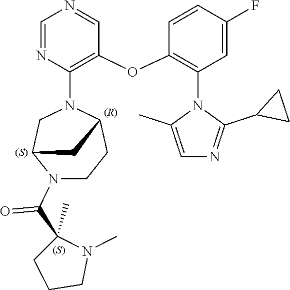

The reductive amination of (6-(5-(2-(2-cyclopropyl-5-methyl-1H-imidazol-1-yl)-4-fluorophenoxy)pyrimidin-4-yl)-2,6-diazabicyclo[3.2.1]octan-2-yl)((S)-2-methylpyrrolidin-2-yl)methanone (30 mg, 0.06 mmol) was performed according to general procedure H1, with the addition of AcOH (0.01 mL). The reaction mixture was diluted with water (10 mL), and extracted with DCM (3×10 mL). The combined organic layers were washed with brine (9 mL), dried over Na₂SO₄, and after evaporation, the residue was purified using method Prep-HPLC1 (Gradient: 26% B to 51% B in 8 min; RT1(min): 7.23). This yielded ((1S,5R)-6-(5-(2-(2-cyclopropyl-5-methyl-1H-imidazol-1-yl)-4-fluorophenoxy)pyrimidin-4-yl)-2,6-diazabicyclo[3.2.1]octan-2-yl)((S)-1,2-dimethylpyrrolidin-2-yl)methanone (8 mg, 24%) as a colourless solid.

¹H NMR (400 MHz, DMSO-d₆, 300 K) 68.33 (s, 1H), 7.90 (s, 1H), 7.59 (dd, J=8.8 Hz, 2.8 Hz, 1H), 7.51-7.32 (m, 1H), 7.30-6.89 (m, 1H), 6.72-6.45 (m, 1H), 6.20-4.88 (m, 1H), 4.87-3.88 (m, 2H), 3.85-3.56 (m, 1H), 3.28-3.27 (m, 3H), 3.08-2.78 (m, 1H), 2.48-2.38 (m, 1H), 2.25-2.00 (m, 4H), 1.95 (s, 3H), 1.90-1.70 (m, 3H), 1.70-1.59 (m, 2H), 1.58-1.42 (m, 2H), 1.02 (s, 3H), 0.88-0.58 (m, 4H). m/z: ES⁺ [M+H]⁺=545.25.

### Example 6.04: ((1S,5R)-6-(6-(2-(2-cyclopropyl-5-methyl-1H-imidazol-1-yl)-4-fluorophenoxy)-1,2,4-triazin-5-yl)-2,6-diazabicyclo[3.2.1]octan-2-yl)((S)-1,2-dimethylpyrrolidin-2-yl)methanone

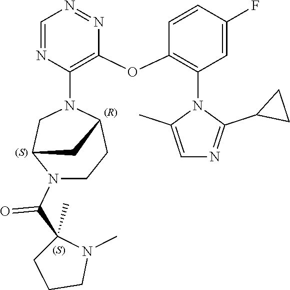

The reductive amination of ((1S,5R)-6-(6-(2-(2-cyclopropyl-5-methyl-1H-imidazol-1-yl)-4-fluorophenoxy)-1,2,4-triazin-5-yl)-2,6-diazabicyclo[3.2.1]octan-2-yl)((S)-2-methylpyrrolidin-2-yl)methanone (90 mg, 0.17 mmol) was performed according to general procedure H1, with the addition of AcOH (0.1 mL). The reaction mixture was filtered; the filter cake was washed with MeOH (2 mL), and the filtrate was concentrated under reduced pressure. The residue was purified by using method RPCF1 (10% to 50% gradient in 10 min), affording ((1S,5R)-6-(6-(2-(2-cyclopropyl-5-methyl-1H-imidazol-1-yl)-4-fluorophenoxy)-1,2,4-triazin-5-yl)-2,6-diazabicyclo[3.2.1]octan-2-yl)((S)-1,2-dimethylpyrrolidin-2-yl)methanone (53 mg, 58%) as a colourless solid.

¹H NMR (400 MHz, DMSO-d₆, 300 K) δ 8.33 (s, 1H), 7.90 (s, 1H), 7.59 (dd, J=8.8 Hz, 2.8 Hz, 1H), 7.51-7.32 (m, 1H), 7.30-6.89 (m, 1H), 6.72-6.45 (m, 1H), 6.20-4.88 (m, 1H), 4.87-3.88 (m, 2H), 3.85-3.56 (m, 1H), 3.28-3.27 (m, 3H), 3.08-2.78 (m, 1H), 2.48-2.38 (m, 1H), 2.25-2.00 (m, 4H), 1.95 (s, 3H), 1.90-1.70 (m, 3H), 1.70-1.59 (m, 2H), 1.58-1.42 (m, 2H), 1.02 (s, 3H), 0.88-0.58 (m, 4H). m/z: ES⁺ [M+H]⁺=545.15.

### Example 6.05: N-ethyl-5-fluoro-N-isopropyl-2-((4-((1S,5R)-2-(2-((R)-1-methylpyrrolidin-2-yl)acetyl)-2,6-diazabicyclo[3.2.1]octan-6-yl)pyrimidin-5-yl)oxy)benzamide

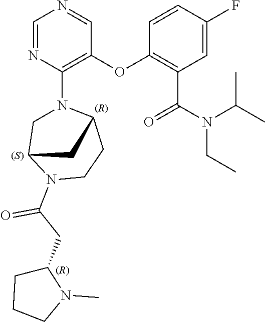

The reductive amination of N-ethyl-5-fluoro-N-isopropyl-2-((4-((1S,5R)-2-(2-((R)-pyrrolidin-2-yl)acetyl)-2,6-diazabicyclo[3.2.1]octan-6-yl)pyrimidin-5-yl)oxy)benzamide (105 mg, 0.2 mmol) was performed according to general procedure H1. The reaction mixture was concentrated under reduced pressure, the residue was extracted with DCM (2×5 mL) and the combined organic layers were washed with brine, dried over Na₂SO₄, filtered and concentrated under reduced pressure. The crude product was purified using method Prep-HPLC1 (Gradient: 18% B to 39% B in 7 min; RT1(min): 7.67) to afford N-ethyl-5-fluoro-N-isopropyl-2-((4-((1S,5R)-2-(2-((R)-1-methylpyrrolidin-2-yl)acetyl)-2,6-diazabicyclo[3.2.1]octan-6-yl)pyrimidin-5-yl)oxy)benzamide (32 mg, 29%) as a colourless solid.

¹H NMR (400 MHz, DMSO-d₆, 300 K) δ 8.34-8.31 (m, 1H), 7.84-7.76 (m, 1H), 7.29-7.22 (m, 2H), 7.08-6.91 (m, 1H), 5.15-4.66 (m, 2H), 4.22-3.73 (m, 3H), 3.66-3.37 (m, 1H), 3.39-3.34 (m, 1H), 3.20-3.00 (m, 2H), 2.90-2.88 (m, 1H), 2.89-2.61 (m, 1H), 2.50-2.30 (m, 1H), 2.28-2.10 (m, 3H), 2.08-2.04 (m, 2H), 1.92-1.88 (m, 3H), 1.78-1.48 (m, 4H), 1.33-1.20 (m, 1H), 1.17-0.98 (m, 9H). m/z: ES⁺ [M+H]⁺=539.3.

### Example 6.06: N-ethyl-5-fluoro-N-isopropyl-2-((4-((1S,5R)-2-(2-(1-methylazetidin-3-yl)acetyl)-2,6-diazabicyclo[3.2.1]octan-6-yl)pyrimidin-5-yl)oxy)benzamide

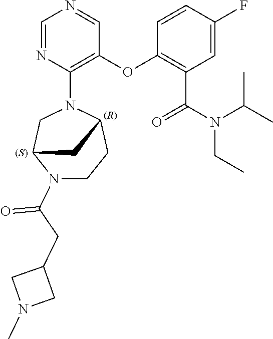

A mixture of 2-((4-((1S,5R)-2-(2-(azetidin-3-yl)acetyl)-2,6-diazabicyclo[3.2.1]octan-6-yl)pyrimidin-5-yl)oxy)-N-ethyl-5-fluoro-N-isopropylbenzamide (60 mg, 0.12 mmol, 1.0 equiv) and methyl trinfluoromethanesulfonate (57 mg, 0.35 mmol, 3.0 equiv) in HFIP (2 mL) was stirred at room temperature for 2 h under nitrogen and then concentrated to give a crude residue that was purified using method RPCF2 (10% to 50% gradient in 10 min) to afford N-ethyl-5-fluoro-N-isopropyl-2-((4-((1S,5R)-2-(2-(1-methylazetidin-3-yl)acetyl)-2,6-diazabicyclo[3.2.1]octan-6-yl)pyrimidin-5-yl)oxy)benzamide (10 mg, 11%) as a colourless oil.

¹H NMR (400 MHz, DMSO-d₆, 300 K) δ 8.95-8.55 (m, 1H), 8.37-8.05 (m, 1H), 7.62-6.95 (m, 3H), 5.35-4.95 (m, 1H), 4.82-4.65 (m, 1H), 4.35-4.15 (m, 1H), 3.98-3.92 (m, 2H), 3.85-3.72 (m, 5H), 3.72-3.65 (m, 2H), 3.25-3.15 (m, 3H), 3.05-2.75 (m, 2H), 2.55-2.45 (m, 2H), 2.10-1.72 (m, 4H), 1.21-1.12 (m, 3H), 1.10-1.00 (m, 5H), 0.99-0.85 (m, 1H). m/z: ES⁺ [M+H]⁺=525.4.

### Example 6.07: ((1S,5R)-6-(6-(2-(2-cyclopropyl-5-methyl-1H-imidazol-1-yl)-4-fluorophenoxy)-1,2,4-triazin-5-yl)-2,6-diazabicyclo[3.2.1]octan-2-yl)(7-methyl-7-azabicyclo[2.2.1]heptan-1-yl)methanone

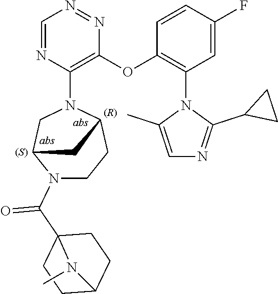

The reductive amination of (7-azabicyclo[2.2.1]heptan-1-yl)((1S,5R)-6-(6-(2-(2-cyclopropyl-5-methyl-1H-imidazol-1-yl)-4-fluorophenoxy)-1,2,4-triazin-5-yl)-2,6-diazabicyclo[3.2.1]octan-2-yl)methanone (70 mg, 0.129 mmol, 1.0 equiv) was performed according to general procedure H1. The crude product was diluted with MeOH (1 mL) then was purified using method Prep-HPLC1 (Gradient: 25% B to 50% B in 7 min; RT1(min): 6.27) to afford ((1S,5R)-6-(6-(2-(2-cyclopropyl-5-methyl-1H-imidazol-1-yl)-4-fluorophenoxy)-1,2,4-triazin-5-yl)-2,6-diazabicyclo[3.2.1]octan-2-yl)(7-methyl-7-azabicyclo[2.2.1]heptan-1-yl)methanone (5 mg, 7%) as a colourless solid.

¹H NMR (400 MHz, DMSO-d₆, 300 K) δ 8.48 (s, 1H), 7.68-7.53 (m, 3H), 6.64-6.14 (m, 2H), 5.21 (s, 1H), 4.79-4.74 (m, 1H), 4.49-4.19 (m, 1H), 3.91-3.42 (m, 2H), 3.03-2.60 (m, 1H), 2.10-1.96 (m, 9H), 1.88-1.84 (m, 3H), 1.75-1.62 (m, 4H), 1.41-1.31 (m, 3H), 0.77-0.25 (m, 4H). m/z: ES⁺ [M+H]⁺=559.05.

### Example 6.08: ((1S,5R)-6-(6-(2-(2,5-dicyclopropyl-1H-imidazol-1-yl)-4-fluorophenoxy)-1,2,4-triazin-5-yl)-2,6-diazabicyclo[3.2.1]octan-2-yl)((S)-1,2-dimethylpyrrolidin-2-yl)methanone

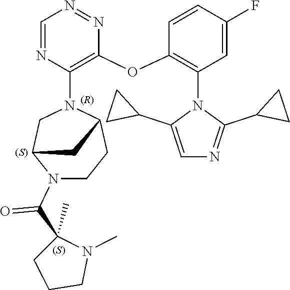

The reductive amination of ((1S,5R)-6-(6-(2-(2,5-dicyclopropyl-1H-imidazol-1-yl)-4-fluorophenoxy)-1,2,4-triazin-5-yl)-2,6-diazabicyclo[3.2.1]octan-2-yl)((S)-2-methylpyrrolidin-2-yl)methanone (30 mg, 0.05 mmol) was performed according to general procedure H1. The residue was purified using method RPFC5 (Gradient: 1% B to 15% B in 7 min) to afford ((1S,5R)-6-(6-(2-(2,5-dicyclopropyl-1H-imidazol-1-yl)-4-fluorophenoxy)-1,2,4-triazin-5-yl)-2,6-diazabicyclo[3.2.1]octan-2-yl)((S)-1,2-dimethylpyrrolidin-2-yl)methanone (7 mg, 23%) as a colourless solid.

¹H NMR (400 MHz, DMSO-d₆, 300 K) δ 8.62-8.32 (m, 1H), 7.85-7.60 (m, 2H), 8.60-7.36 (m, 1H), 6.55-6.23 (m, 1H), 6.12-4.90 (m, 1H), 4.85-4.54 (m, 1H), 4.53-3.95 (m, 1H), 3.90-3.66 (m, 1H), 3.63-3.46 (m, 1H), 3.02-2.57 (m, 2H), 2.41-2.43 (m, 1H), 2.23-1.99 (m, 4H), 1.95-1.71 (m, 4H), 1.71-1.49 (m, 3H), 1.48-1.23 (m, 2H), 1.08-0.94 (m, 3H), 0.79-0.26 (m, 8H). m/z: ES⁺ [M+H]⁺=573.30.

### Example 6.09 and 6.10: ((1S,5R)-6-(6-(2-(2,5-dicyclopropyl-1H-imidazol-1-yl)-4-fluorophenoxy)-1,2,4-triazin-5-yl)-2,6-diazabicyclo[3.2.1]octan-2-yl)((3aR,6aR)-2-methylhexahydrocyclopenta[c]pyrrol-3a(1H)-yl)methanone and ((1S,5R)-6-(6-(2-(2,5-dicyclopropyl-1H-imidazol-1-yl)-4-fluorophenoxy)-1,2,4-triazin-5-yl)-2,6-diazabicyclo[3.2.1]octan-2-yl)((3aS,6aS)-2-methylhexahydrocyclopenta[c]pyrrol-3a(1H)-yl)methanone

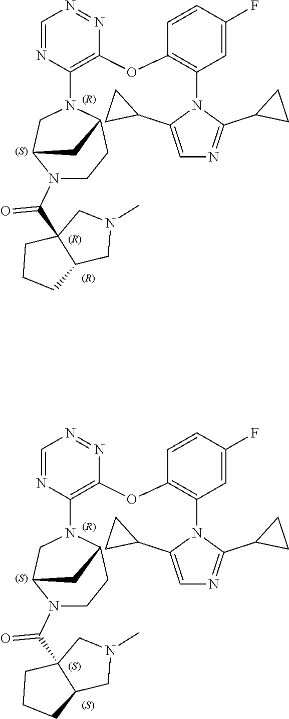

The reductive amination of ((1S,5R)-6-(6-(2-(2,5-dicyclopropyl-1H-imidazol-1-yl)-4-fluorophenoxy)-1,2,4-triazin-5-yl)-2,6-diazabicyclo[3.2.1]octan-2-yl)((3aRS,6aRS)-hexahydrocyclopenta[c]pyrrol-3a(1H)-yl)methanone (50 mg, 0.09 mmol) was performed according to general procedure H1. The residue was purified using method Prep-HPLC1 (Gradient: 25% B to 50% B in 7 min; RT1(min): 6.38) to afford ((1S,5R)-6-(6-(2-(2,5-dicyclopropyl-1H-imidazol-1-yl)-4-fluorophenoxy)-1,2,4-triazin-5-yl)-2,6-diazabicyclo[3.2.1]octan-2-yl)((3aRS,6aRS)-2-methylhexahydrocyclopenta[c]pyrrol-3a(1H)-yl)methanone (25 mg, 49%) as a colourless solid. The mixture was separated using method CHIRALPAKIM (A: Hex(0.2% IPAmine), B: EtOH:DCM=1:1; gradient: isocratic 50% B).

Peak #1: Rt1=15.7 min, colourless solid (25 mg, 49%).

¹H NMR (400 MHz, DMSO-d₆, 300 K) δ 8.60-8.46 (s, 1H), 7.80-7.67 (m, 2H), 7.65-7.45 (m, 1H), 6.60-5.77 (m, 1H), 5.20-4.46 (m, 2H), 4.36-3.80 (m, 2H), 3.65-3.45 (m, 2H), 3.25-3.05 (m, 1H), 3.03-2.67 (m, 1H), 2.37-2.27 (m, 2H), 2.26-2.08 (m, 4H), 1.98-1.78 (m, 3H), 1.76-1.48 (m, 5H), 1.46-1.36 (m, 4H), 1.24-1.14 (m, 1H). 0.84-0.64 (m, 3H). 0.59-0.45 (m, 4H). m/z: ES⁺ [M+H]⁺=599.30.

Peak #2: Rt2=18.7 min, colourless solid (4 mg, 9%).

¹H NMR (400 MHz, DMSO-d₆, 300 K) δ 8.60-8.46 (s, 1H), 7.80-7.67 (m, 2H), 7.65-7.45 (m, 1H), 6.60-5.77 (m, 1H), 5.20-4.46 (m, 2H), 4.46-3.50 (m, 3H), 3.45-3.35 (m, 1H), 3.25-2.95 (m, 1H), 2.90-2.67 (m, 2H), 2.47-2.27 (m, 1H), 2.26-2.08 (m, 4H), 1.98-1.55 (m, 8H), 1.46-1.36 (m, 4H), 0.84-0.64 (m, 4H). 0.59-0.45 (m, 4H). m/z: ES⁺ [M+H]⁺=599.30.

### Example 6.11: ((1S,5R)-6-(6-(2-(2,5-dicyclopropyl-1H-imidazol-1-yl)-4-fluorophenoxy)-1,2,4-triazin-5-yl)-2,6-diazabicyclo[3.2.1]octan-2-yl)((S)-1-ethyl-2-methylpyrrolidin-2-yl)methanone

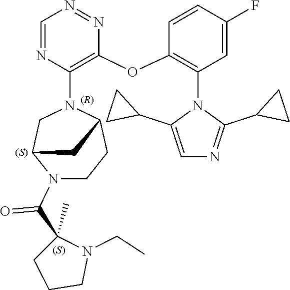

The reductive amination of ((1S,5R)-6-(6-(2-(2,5-dicyclopropyl-1H-imidazol-1-yl)-4-fluorophenoxy)-1,2,4-triazin-5-yl)-2,6-diazabicyclo[3.2.1]octan-2-yl)((S)-2-methylpyrrolidin-2-yl)methanone (25 mg, 0.045 mmol) was performed according to general procedure H1. The residue was purified using method Prep-HPLC6 (Gradient: 2% B to 30% B in 8 min) to afford ((1S,5R)-6-(6-(2-(2,5-dicyclopropyl-1H-imidazol-1-yl)-4-fluorophenoxy)-1,2,4-triazin-5-yl)-2,6-diazabicyclo[3.2.1]octan-2-yl)((S)-1,2-dimethylpyrrolidin-2-yl)methanone (2 mg, 9%) as an off-white solid.

¹H NMR (400 MHz, DMSO-d₆, 300 K) δ 8.70-8.61 (m, 1H), 7.77-7.61 (m, 2H), 7.60-7.51 (m, 1H), 6.50-6.41 (m, 1H), 6.35-4.90 (m, 1H), 4.85-4.45 (m, 1H), 4.44-3.90 (m, 1H), 3.89-3.75 (m, 1H), 3.70-3.45 (m, 1H), 3.21-3.01 (m, 1H), 2.45-2.21 (m, 3H), 2.10-1.75 (m, 5H), 1.70-1.25 (m, 6H), 1.11-0.95 (m, 6H), 0.85-0.61 (m, 3H), 0.60-0.25 (m, 5H). m/z: ES⁺ [M+H]⁺=587.30.

### Examples 6.12 and 6.13: N-ethyl-5-fluoro-N-isopropyl-2-(4-((1S,5R)-2-((R)-2-((R)-1-methylpyrrolidin-3-yl)propanoyl)-2,6-diaza-bicyclo[3.2.1]octan-6-yl)pyrimidin-5-yloxy)benzamide and N-ethyl-5-fluoro-N-isopropyl-2-(4-((1S,5R)-2-((S)-2-((R)-1-methylpyrrolidin-3-yl)propanoyl)-2,6-diaza-bicyclo[3.2.1]octan-6-yl)pyrimidin-5-yloxy)benzamide

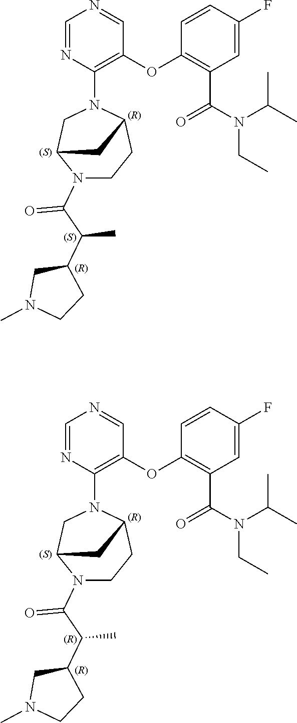

The reductive amination of N-ethyl-5-fluoro-N-isopropyl-2-((4-((1S,5R)-2-((RS)-2-((R)-pyrrolidin-3-yl)propanoyl)-2,6-diazabicyclo[3.2.1]octan-6-yl)pyrimidin-5-yl)oxy)benzamide (25 mg, 0.045 mmol) was performed according to general procedure H1. The residue was purified by prep-TLC (PE/DCM, 1:1) to afford N-ethyl-5-fluoro-N-isopropyl-2-((4-((1S,5R)-2-((RS)-2-((R)-1-methylpyrrolidin-3-yl)propanoyl)-2,6-diazabicyclo[3.2.1]octan-6-yl)pyrimidin-5-yl)oxy)benzamide, which was separated by chiral HPLC using method CHIRALPAKIM (A: Hex(0.2% IPamine), B: EtOAc:DCM=1:1; gradient: isocratic 30%).

Peak #1: Rt1=6.45 min, colourless solid (29 mg, 3%).

¹H NMR (400 MHz, DMSO-d₆, 300 K) δ 8.42-8.21 (m, 1H), 7.95-7.75 (m, 1H), 7.35-7.15 (m, 2H), 7.10-6.92 (m, 1H), 5.21-4.51 (m, 2H), 4.45-4.05 (m, 1H), 3.95-3.75 (m, 3H), 3.74-3.65 (m, 1H), 3.60-3.55 (m, 1H), 3.40-3.33 (m, 2H), 3.30-3.25 (m, 1H), 3.21-3.05 (m, 1H), 2.92-2.70 (m, 4H), 2.41-2.11 (m, 5H), 2.05-1.80 (m, 3H), 1.74-1.45 (m, 3H), 1.25-1.20 (m, 1H), 1.15-1.05 (m, 3H), 1.04-0.95 (m, 5H), 0.97-0.82 (m, 2H).

m/z: ES⁺ [M+H]⁺=553.55.

Peak #2: Rt2=10.29 min, colourless solid (14 mg, 2%).

¹H NMR (400 MHz, DMSO-d₆, 300 K) δ 8.42-8.21 (m, 1H), 7.95-7.75 (m, 1H), 7.35-7.15 (m, 2H), 7.10-6.92 (m, 1H), 5.21-4.81 (m, 1H), 4.80-4.51 (m, 1H), 4.45-4.05 (m, 1H), 3.95-3.75 (m, 3H), 3.34-3.05 (m, 3H), 2.92-2.50 (m, 3H), 2.41-2.11 (m, 6H), 2.05-1.40 (m, 5H), 1.30-1.15 (m, 2H), 1.14-1.00 (m, 8H), 0.97-0.82 (m, 3H). m/z: ES⁺ [M+H]⁺=553.55.

### Example 6.14: N-ethyl-5-fluoro-N-isopropyl-2-((5-((1S,5R)-2-((3aS,6aS)-2-methyloctahydrocyclopenta[c]pyrrole-3a-carbonyl)-2,6-diazabicyclo[3.2.1]octan-6-yl)-1,2,4-triazin-6-yl)oxy)benzamide

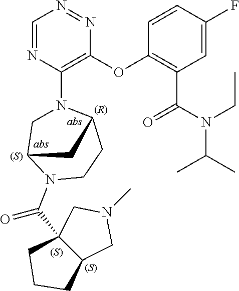

The reductive amination of N-ethyl-5-fluoro-N-isopropyl-2-((5-((1S,5R)-2-((3aS,6aS)-octahydrocyclopenta[c]pyrrole-3a-carbonyl)-2,6-diazabicyclo[3.2.1]octan-6-yl)-1,2,4-triazin-6-yl)oxy)benzamide (160 mg, 0.29 mmol, 1.0 equiv) was performed according to general procedure H1. The reaction was quenched with sat.

NH₄Cl (50 mL), and extracted with EtOAc (3×100 mL). Thecombined organic fractions were dried over Na₂SO₄, filtered and concentrated under reduced pressure to give a residue that was purified using method Prep-HPLC1 (Gradient: 23% B to 53% B in 8 min; Rt=6.57 min) to afford N-ethyl-5-fluoro-N-isopropyl-2-((5-((1S,5R)-2-((3aS,6aS)-2-methyloctahydrocyclopenta[c]pyrrole-3a-carbonyl)-2,6-diazabicyclo[3.2.1]octan-6-yl)-1,2,4-triazin-6-yl)oxy)benzamide (36 mg, 22%) as a colourless solid.

¹H NMR (400 MHz, DMSO-d₆, 300 K) δ 8.65-8.47 (m, 1H), 7.47-7.40 (m, 1H), 7.39-7.34 (m, 2H), 5.32-4.89 (m, 1H), 4.83-4.68 (m, 1H), 4.27-3.57 (m, 4H), 3.52-3.47 (m, 1H), 3.24-2.91 (m, 2H), 2.89-2.64 (m, 2H), 2.53-2.36 (m, 1H), 2.29-2.14 (m, 4H), 2.09-1.84 (m, 3H), 1.81-1.62 (m, 5H), 1.63-1.34 (m, 2H), 1.16-1.02 (m, 4H), 1.01-0.84 (m, 4H), 0.80-0.62 (m, 2H). m/z: ES⁺ [M+H]⁺=566.30.

### Example 6.15: ((1S,5R)-6-(6-(2-(2,5-dicyclopropyl-1H-imidazol-1-yl)-4-fluorophenoxy)-1,2,4-triazin-5-yl)-2,6-diazabicyclo[3.2.1]octan-2-yl)((3aR,6aR)-2-methylhexahydrocyclopenta[c]pyrrol-3a(1H)-yl)methanone and ((1S,5R)-6-(6-(2-(2,5-dicyclopropyl-1H-imidazol-1-yl)-4-fluorophenoxy)-1,2,4-triazin-5-yl)-2,6-diazabicyclo[3.2.1]octan-2-yl)((3aS,6aS)-2-methylhexahydrocyclopenta[c]pyrrol-3a(1H)-yl)methanone

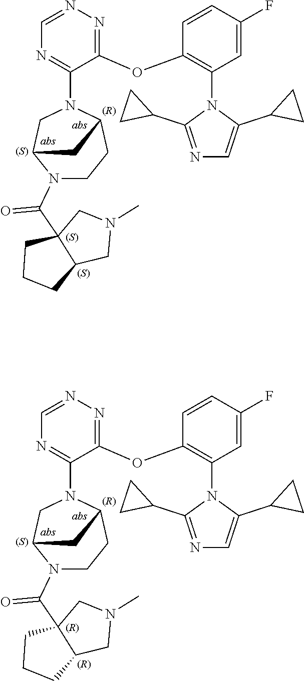

The reductive amination of the mixture of ((1S,5R)-6-(6-(2-(2,5-dicyclopropyl-1H-imidazol-1-yl)-4-fluorophenoxy)-1,2,4-triazin-5-yl)-2,6-diazabicyclo[3.2.1]octan-2-yl)((3aS,6aS)-2-methylhexahydrocyclopenta[c]pyrrol-3a(1H)-yl)methanone and ((1S,5R)-6-(6-(2-(2,5-dicyclopropyl-1H-imidazol-1-yl)-4-fluorophenoxy)-1,2,4-triazin-5-yl)-2,6-diazabicyclo[3.2.1]octan-2-yl)((3aR,6aR)-2-methylhexahydrocyclopenta[c]pyrrol-3a(1H)-yl)methanone (10 mg, 0.02 mmol) was performed according to general procedure H1. The residue was purified using method Prep-HPLC6 to afford the title mixture as a colourless solid (2 mg, 22%).

¹H NMR (400 MHz, DMSO-d₆, 300K) δ 8.60-8.46 (m, 1H), 7.80-7.67 (m, 2H), 7.65-7.45 (m, 1H), 6.60-5.77 (m, 1H), 5.20-4.46 (m, 2H), 4.46-3.50 (m, 3H), 3.45-3.35 (m, 1H), 3.25-2.95 (m, 1H), 2.90-2.67 (m, 2H), 2.47-2.27 (m, 1H), 2.26-2.08 (m, 4H), 1.98-1.55 (m, 8H), 1.46-1.36 (m, 4H), 0.84-0.64 (m, 4H). 0.59-0.45 (m, 4H). m/z: ES⁺ [M+H]⁺=599.35.

### Example 6.16 and 6.17: ((1S,5R)-6-(6-(2-(2-cyclopropyl-5-methyl-1H-imidazol-1-yl)-4-fluorophenoxy)-1,2,4-triazin-5-yl)-2,6-diazabicyclo[3.2.1]octan-2-yl)((3aR,6aR)-2-methylhexahydrocyclopenta[c]pyrrol-3a(1H)-yl)methanone and ((1S,5R)-6-(6-(2-(2-cyclopropyl-5-methyl-1H-imidazol-1-yl)-4-fluorophenoxy)-1,2,4-triazin-5-yl)-2,6-diazabicyclo[3.2.1]octan-2-yl)((3aS,6aS)-2-methylhexahydrocyclopenta[c]pyrrol-3a(1H)-yl)methanone

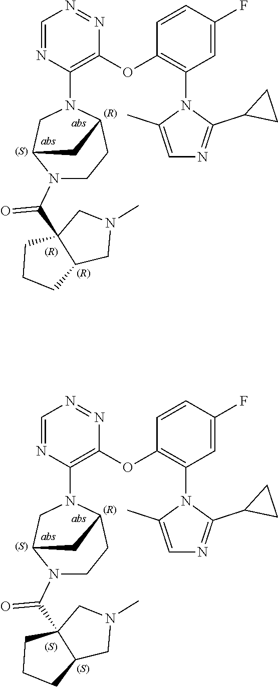

The reductive amination of ((1S,5R)-6-(6-(2-(2-cyclopropyl-5-methyl-1H-imidazol-1-yl)-4-fluorophenoxy)-1,2,4-triazin-5-yl)-2,6-diazabicyclo[3.2.1]octan-2-yl)((3a,6a)-hexahydrocyclopenta[c]pyrrol-3a(1H)-yl)methanone (300 mg, 0.54 mmol, 1.0 equiv) was performed according to general procedure H1. The resulting mixture was diluted with water (10 mL), and extracted with EtOAc (3×10 mL). The combined organic layers were washed with brine (10 mL), dried over Na₂SO₄, and concentrated under reduced pressure. The residue was purified using method RPFC2 (40% to 50% gradient in 10 min) to afford ((1S,5R)-6-(6-(2-(2-cyclopropyl-5-methyl-1H-imidazol-1-yl)-4-fluorophenoxy)-1,2,4-triazin-5-yl)-2,6-diazabicyclo[3.2.1]octan-2-yl)((3aRS,6aRS)-2-methylhexahydrocyclopenta[c]pyrrol-3a(1H)-yl)methanone (100 mg, 33%) as a light yellow oil. m/z: ES⁺ [M+H]⁺=573.30.

Separation by chiral HPLC (Method: CHIRALPAKIM) gave two single stereoisomers:

Peak #1 (RT1: 22.12 min): Off-white solid (28 mg, 7% over 4 steps)

¹H NMR (400 MHz, DMSO-d₆) δ 8.61-8.40 (m, 1H), 8.31 (s, 1H), 7.81-7.59 (m, 2H), 7.58-7.45 (m, 1H), 6.65-6.12 (m, 1H), 5.25-5.03 (m, 1H), 4.86-4.60 (m, 1H), 4.25-3.95 (m, 1H), 3.80-3.67 (m, 1H), 3.59-3.50 (m, 1H), 3.43-3.38 (m, 2H), 3.29-3.21 (m, 1H), 2.80-2.69 (m, 1H), 2.44-2.40 (m, 1H), 2.29-2.19 (m, 1H), 2.19-2.07 (m, 3H), 1.95-1.78 (m, 6H), 1.73-1.53 (m, 4H), 1.51-1.31 (m, 3H), 1.30-1.19 (m, 1H), 1.19-1.10 (m, 2H), 0.78-0.63 (m, 1H), 0.62-0.38 (m, 1H). m/z: ES⁺ [M+H]⁺=573.25.

Peak #2 (RT2: 27.23 min): Off-white solid (18 mg, 4% over 4 steps)

¹H NMR (400 MHz, DMSO-d₆) δ 8.65-8.30 (m, 1H), 7.81-7.60 (m, 2H), 7.58-7.40 (m, 1H), 6.58-6.25 (m, 1H), 5.50-4.00 (m, 2H), 3.98-3.40 (m, 2H), 3.40-3.30 (m, 1H), 3.20-2.90 (m, 1H), 2.88-2.65 (m, 2H), 2.55-2.35 (m, 2H), 2.25-2.10 (m, 4H), 1.99-1.78 (m, 5H), 1.75-1.48 (m, 8H), 1.47-1.20 (m, 2H), 0.90-0.38 (m, 3H). m/z: ES⁺ [M+H]⁺=573.25.

### Example 6.18: ((1S,5R)-6-(6-(2-(5-cyclopropyl-2-methyl-1H-imidazol-1-yl)-4-fluorophenoxy)-1,2,4-triazin-5-yl)-2,6-diazabicyclo[3.2.1]octan-2-yl)((3aR,6aR)-2-methylhexahydrocyclopenta[c]pyrrol-3a(1H)-yl)methanone

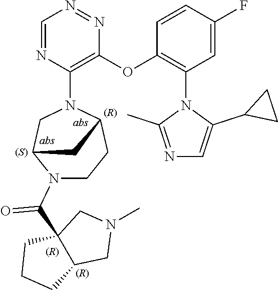

The reductive amination of ((1S,5R)-6-(6-(2-(5-cyclopropyl-2-methyl-1H-imidazol-1-yl)-4-fluorophenoxy)-1,2,4-triazin-5-yl)-2,6-diazabicyclo[3.2.1]octan-2-yl)((3aR,6aR)-hexahydrocyclopenta[c]pyrrol-3a(1H)-yl)methanone (95 mg, 0.17 mmol, 1.0 equiv) was performed according to general procedure H1. The reaction was concentrated under reduced pressure, quenched with water at room temperature and was extracted with DCM. The combined organic layers were washed with brine, dried over Na₂SO₄, and concentrated under reduced pressure. The crude product was purified using method Prep-HPLC1 (Gradient: 28% B to 44% B in 7 min; Rt=6.93 min) to afford ((1S,5R)-6-(6-(2-(5-cyclopropyl-2-methyl-1H-imidazol-1-yl)-4-fluorop-henoxy)-1,2,4-triaz-in-5-yl)-2,6-diazabicyclo[3.2.1]octan-2-yl)((3aR,6aR)-2-methylhexahydrocyclopenta[c]pyrrol-3a(1H)-yl)methanone (27 mg, 28%) as a colourless solid.

¹H NMR (400 MHz, DMSO-d₆, 300 K) δ 8.50-8.46 (m, 1H), 7.68-7.60 (m, 2H), 7.60-7.50 (m, 1H), 6.44-6.39 (m, 1H), 5.15-4.62 (m, 2H), 4.20-3.56 (m, 3H), 3.39-3.35 (m, 1H), 3.14-2.90 (m, 1H), 2.71-2.67 (m, 1H), 2.50-2.41 (m, 1H), 2.21-2.02 (m, 4H), 1.99-1.87 (m, 7H), 1.75-1.62 (m, 5H), 1.44-1.31 (m, 3H), 0.61-0.58 (m, 3H), 0.58-0.37 (m, 1H). m/z: ES⁺ [M+H]⁺=573.35.

### Example 6.19 and 6.20: ((1S,5R)-6-(6-(4-fluoro-2-(2-isopropyl-5-methyl-1H-imidazol-1-yl)phenoxy)-1,2,4-triazin-5-yl)-2,6-diazabicyclo[3.2.1]octan-2-yl)((3aS,6aS)-2-methylhexahydrocyclopenta[c]pyrrol-3a(1H)-yl)methanone and ((1S,5R)-6-(6-(4-fluoro-2-(2-isopropyl-5-methyl-1H-imidazol-1-yl)phenoxy)-1,2,4-triazin-5-yl)-2,6-diazabicyclo[3.2.1]octan-2-yl)((3aR,6aR)-2-methylhexahydrocyclopenta[c]pyrrol-3a(1H)-yl)methanone

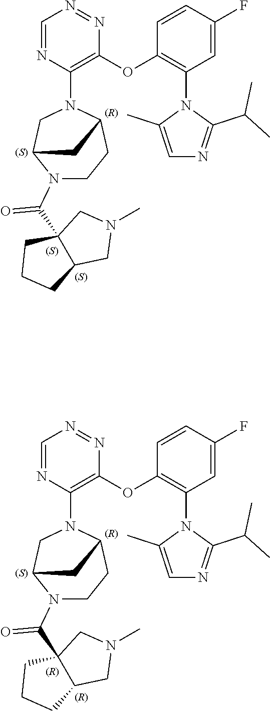

The single stereoisomers of examples 3.23 and 3.24 were subjected to reductive amination according to general procedure H1.

Peak #1: after 1 h reaction time, the resulting mixture was diluted with water (100 mL) and extracted with EtOAc (3×100 mL), dried over Na₂SO₄, and concentrated under reduced pressure. The crude product was purified using method Prep-HPLC8 (Gradient (B %): 27% B to 40% B in 7 min; RT1(min): 7.4) to give an off-white solid (44 mg, 48%).

¹H NMR (400 MHz, DMSO-d₆, 300 K) δ 8.55-8.46 (m, 1H), 7.74-7.56 (m, 3H), 6.72-6.57 (m, 1H), 5.12-4.61 (m, 2H), 4.23-3.76 (m, 3H), 3.55-3.45 (m, 1H), 3.02-2.97 (m, 1H), 2.95-2.19 (m, 9H), 1.98-1.87 (m, 5H), 1.71-1.62 (m, 5H), 1.59-1.40 (m, 2H), 1.05-0.79 (m, 6H). m/z: ES⁺ [M+H]⁺=575.25.

Peak #2: after 1 h reaction time, the resulting mixture was diluted with water (100 mL) and extracted with EtOAc (3×100 mL), dried over Na₂SO₄, and concentrated under reduced pressure. The crude product was purified using method Prep-HPLC1 (Gradient (B %): 24% B to 45% B in 7 min; RT1(min): 6.5) to give an off-white solid (55 mg, 60%).

¹H NMR (400 MHz, DMSO-d₆, 300 K) δ 8.55-8.46 (m, 1H), 7.74-7.56 (m, 3H), 6.72-6.57 (m, 1H), 5.12-4.61 (m, 2H), 4.23-3.76 (m, 2H), 3.55-3.45 (m, 2H), 3.02-2.97 (m, 1H), 2.95-2.19 (m, 9H), 1.98-1.87 (m, 6H), 1.71-1.62 (m, 4H), 1.59-1.40 (m, 2H), 1.05-0.79 (m, 6H). m/z: ES⁺ [M+H]⁺=575.35.

### Example 6.21: N-ethyl-5-fluoro-N-isopropyl-2-((5-((1S,5R)-2-((3aR,6aR)-2-methyloctahydrocyclopenta[c]pyrrole-3a-carbonyl)-2,6-diazabicyclo[3.2.1]octan-6-yl)-1,2,4-triazin-6-yl)oxy)benzamide

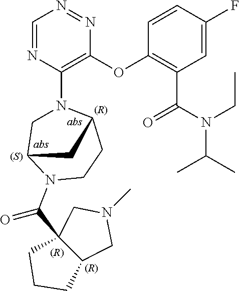

The reductive amination of N-ethyl-5-fluoro-N-isopropyl-2-((5-((1S,5R)-2-((3aR,6aR)-octahy drocy clopenta[c]pyrrole-3a-carbonyl)-2,6-diazabicyclo[3.2.1]octan-6-yl)-1,2,4-triazin-6-yl)oxy)benzamide (170 mg, 0.31 mmol, 1.0 equiv) was performed according to general procedure H1. The reaction was quenched with sat.

NH₄Cl (50 mL), and extracted with EtOAc (3×100 mL). The combined organic fractions were dried over Na₂SO₄, filtered and concentrated under reduced pressure to give a residue that was purified using method Prep-HPLC1 (Gradient: 23% B to 53% B in 8 min; Rt=6.58 min) to afford N-ethyl-5-fluoro-N-isopropyl-2-((5-((1S,5R)-2-((3aR,6aR)-2-methyloctahy drocy clopenta[c]pyrrole-3a-carbonyl)-2,6-diazabicyclo[3.2.1]octan-6-yl)-1,2,4-triazin-6-yl)oxy)benzamide (32 mg, 18%) as a colourless solid.

¹H NMR (400 MHz, DMSO-d₆, 300 K) δ 8.65-8.47 (m, 1H), 7.47-7.40 (m, 1H), 7.39-7.34 (m, 2H), 5.37-5.00 (m, 1H), 4.83-4.68 (m, 1H), 4.37-3.52 (m, 4H), 3.58-3.47 (m, 1H), 3.28-2.95 (m, 2H), 2.92-2.61 (m, 2H), 2.49-2.35 (m, 1H), 2.29-2.11 (m, 4H), 2.17-1.55 (m, 9H), 1.53-1.34 (m, 2H), 1.16-1.02 (m, 4H), 1.01-0.94 (m, 3H), 0.80-0.62 (m, 2H). m/z: ES⁺ [M+H]⁺=566.30.

### Examples 6.22 and 6.23: ((1S,5R)-6-(5-(2-(2-cyclopropyl-5-methyl-1H-imidazol-1-yl)-4-fluorophenoxy)pyrimidin-4-yl)-2,6-diazabicyclo[3.2.1]octan-2-yl)((3aR,6aR)-2-methylhexahydrocyclopenta[c]pyrrol-3a(1H)-yl)methanone and ((1S,5R)-6-(5-(2-(2-cyclopropyl-5-methyl-1H-imidazol-1-yl)-4-fluorophenoxy)pyrimidin-4-yl)-2,6-diazabicyclo[3.2.1]octan-2-yl)((3aS,6aS)-2-methylhexahydrocyclopenta[c]pyrrol-3a(1H)-yl)methanone

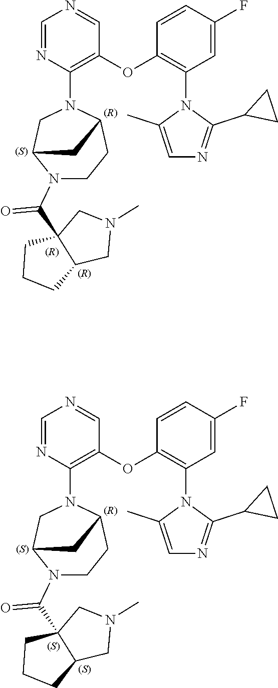

The reductive amination of ((1S,5R)-6-(5-(2-(2-cyclopropyl-5-methyl-1H-imidazol-1-yl)-4-fluorophenoxy)pyrimidin-4-yl)-2,6-diazabicyclo[3.2.1]octan-2-yl)((3aRS,6aRS)-hexahydrocyclopenta[c]pyrrol-3a(1H)-yl)methanone (50 mg, 0.090 mmol, 1.0 equiv) was performed according to general procedure H1. The resulting mixture was concentrated under reduced pressure. The resulting mixture was extracted with DCM. The combined organic layers were washed with brine, dried over Na₂SO₄, and concentrated under reduced pressure. The residue was purified using method RPFC1 (10% to 80% gradient in 25 min) to afford ((1S,5R)-6-(5-(2-(2-cyclopropyl-5-methyl-1H-imidazol-1-yl)-4-fluorophenoxy)pyrimidin-4-yl)-2,6-diazabicyclo[3.2.1]octan-2-yl)((3aRS,6aRS)-2-methylhexahydrocyclopenta[c]pyrrol-3a(1H)-yl)methanone (1.0 g, 79%) as a light yellow solid. m/z: ES⁺ [M+H]⁺=572.30.

Separation by Chiral-Prep-HPLC (Column: CHIRALPAK IM, Mobile Phase A: Hex(0.5% 2M NH₃-MeOH), Mobile Phase B: EtOH/DCM=1/1; Gradient (B %): 20% B to 20% B in 40 min) gave ((1S,5R)-6-(5-(2-(2-cyclopropyl-5-methyl-1H-imidazol-1-yl)-4-fluorophenoxy)pyrimidin-4-yl)-2,6-diazabicyclo[3.2.1]octan-2-yl)((3aR,6aR)-2-methylhexahydrocyclopenta[c]pyrrol-3a(1H)-yl)methanone (217 mg, 22%) as an off-white solid.

¹H NMR (400 MHz, DMSO-d₆, 300K) δ 8.34 (s, 1H), 7.95-7.80 (m, 1H), 7.65-7.55 (m, 1H), 7.45-7.35 (m, 1H), 7.25-7.10 (m, 1H), 6.65-6.55 (m, 1H), 5.06-4.57 (m, 2H), 4.15-3.75 (m, 1H), 3.67-3.45 (m, 2H), 3.32-3.01 (m, 1H), 2.92-2.66 (m, 3H), 2.50-2.17 (m, 5H), 1.99-1.93 (m, 6H), 1.84-1.64 (m, 6H), 1.52-1.45 (m, 2H), 1.38-0.73 (m, 4H). m/z: ES⁺ [M+H]⁺=572.30.

### Examples 6.24, 6.25, 6.26 and 6.27: stereoisomers of ((1S,5R)-6-(6-(2-(2-cyclopropyl-5-isopropyl-1H-imidazol-1-yl)-4-fluorophenoxy)-1,2,4-triazin-5-yl)-2,6-diazabicyclo[3.2.1]octan-2-yl)((3aS,6aS)-2-methylhexahydrocyclopenta[c]pyrrol-3a(1H)-yl)methanone and ((1S,5R)-6-(6-(2-(2-cyclopropyl-5-isopropyl-1H-imidazol-1-yl)-4-fluorophenoxy)-1,2,4-triazin-5-yl)-2,6-diazabicyclo[3.2.1]octan-2-yl)((3aR,6aR)-2-methylhexahydrocyclopenta[c]pyrrol-3a(1H)-yl)methanone

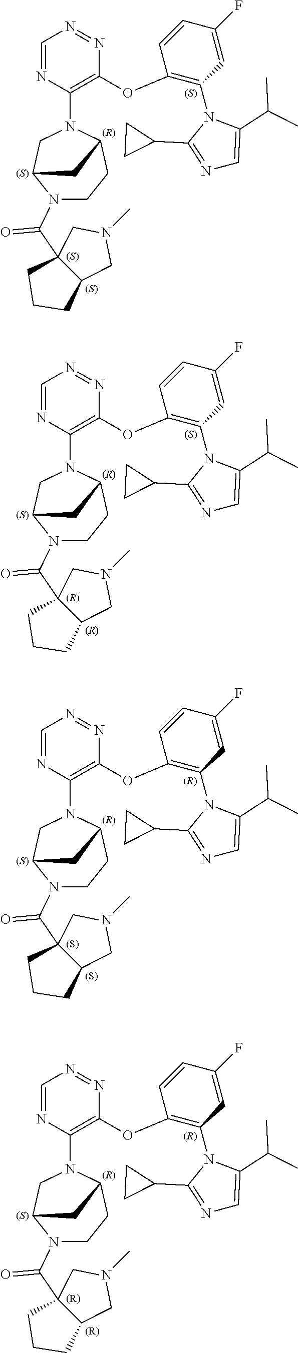

The reductive amination of the mixture of ((1S,5R)-6-(6-(2-(2-cyclopropyl-5-isopropyl-1H-imidazol-1-yl)-4-fluorophenoxy)-1,2,4-triazin-5-yl)-2,6-diazabicyclo[3.2.1]octan-2-yl)((3aS,6aS)-hexahydrocyclopenta[c]pyrrol-3a(1H)-yl)methanone and ((1S,5R)-6-(6-(2-(2-cyclopropyl-5-isopropyl-1H-imidazol-1-yl)-4-fluorophenoxy)-1,2,4-triazin-5-yl)-2,6-diazabicyclo[3.2.1]octan-2-yl)((3aR,6aR)-hexahydrocyclopenta[c]pyrrol-3a(1H)-yl)methanone (120 mg, 0.21 mmol) was performed according to general procedure H1. The residue was purified using method Prep-HPLC6 to afford the title mixture as a light yellow formic acid (5 mg, 5%).

¹H NMR (400 MHz, DMSO-d₆, 300 K) δ 8.561-8.40 (m, 1H), 8.40-7.81 (m, 1H), 784-7.29 (m, 3H), 6.91-6.16 (m, 1H), 5.31-3.97 (m, 3H), 3.62-3.43 (m, 3H), 3.43-2.78 (m, 5H), 2.78-2.56 (m, 2H), 2.12-1.37 (m, 11H), 1.37-1.19 (m, 1H), 1.19-1.50 (m, 8H), 1.50-1.09 (m, 3H). m/z: ES⁺ [M+H]⁺=601.30.

Further separation of 100 mg of the above mixture by chiral HPLC (CHIRALPAK IM isocratic Hex(0.2% IPAmine): (EtOH:DCM=1:1)=70: 30) gave four single stable stereoisomers:

Peak #1: Rt1=13.07 min, colourless solid (5 mg, 5%).

¹H NMR (400 MHz, DMSO-d₆, 300 K) δ 8.56-8.21 (m, 1H), 7.84-7.68 (m, 2H), 7.63-7.47 (m, 1H), 6.65-6.21 (m, 1H), 5.19-4.70 (m, 1H), 4.70-4.13 (m, 1H), 3.86-3.77 (m, 1H), 3.69-3.48 (m, 1H), 3.29-3.11 (m, 1H), 3.02-2.83 (m, 2H), 2.67-2.62 (m, 1H), 2.38-2.21 (m, 4H), 2.02-1.58 (m, 9H), 1.58-1.38 (m, 3H), 1.38-1.12 (m, 2H), 1.11-1.00 (m, 1H), 1.00-0.92 (m, 2H), 0.89-0.76 (m, 3H), 0.71-0.48 (m, 2H), 0.48-0.31 (m, 2H). m/z: ES⁺ [M+H]⁺=601.35.

Peak #2: Rt2=15.04 min, colourless solid (7 mg, 7%).

¹H NMR (400 MHz, DMSO-d₆, 300 K) δ 8.56-8.21 (m, 1H), 7.84-7.68 (m, 2H), 7.63-7.47 (m, 1H), 6.65-6.21 (m, 1H), 5.21-4.49 (m, 2H), 4.33-4.01 (m, 1H), 3.93-3.58 (m, 2H), 3.22-3.11 (m, 1H), 3.02-2.63 (m, 3H), 2.63-2.59 (m, 1H), 2.49-2.41 (m, 1H), 2.38-2.17 (m, 3H), 2.01-1.36 (m, 10H), 1.36-1.15 (m, 2H), 1.11-0.92 (m, 3H), 0.92-0.70 (m, 3H), 0.70-0.31 (m, 4H). m/z: ES⁺ [M+H]⁺=601.35.

Peak #3: Rt3=18.27 min, colourless solid (7 mg, 7%).

¹H NMR (400 MHz, DMSO-d₆, 300 K) δ 8.56-8.21 (m, 1H), 7.84-7.68 (m, 2H), 7.63-7.47 (m, 1H), 6.65-6.31 (m, 1H), 5.21-4.49 (m, 2H), 4.33-4.01 (m, 1H), 3.93-3.78 (m, 1H), 3.22-3.11 (m, 1H), 2.82-2.63 (m, 1H), 2.58-2.49 (m, 2H), 2.32-2.06 (m, 4H), 2.06-1.77 (m, 4H), 1.77-1.52 (m, 4H), 1.52-1.38 (m, 3H), 1.38-1.14 (m, 3H), 1.11-1.01 (m, 3H), 1.01-0.88 (m, 3H), 0.68-0.54 (m, 1H), 0.54-0.22 (m, 3H). m/z: ES⁺ [M+H]⁺=601.30.

Peak #4: Rt4=20.87 min, colourless solid (3 mg, 3%).

¹H NMR (400 MHz, DMSO-d₆, 300 K) δ 8.56-8.21 (m, 1H), 7.84-7.68 (m, 2H), 7.63-7.47 (m, 1H), 6.65-6.21 (m, 1H), 5.29-4.33 (m, 2H), 4.21-3.62 (m, 3H), 3.19-3.02 (m, 1H), 3.02-2.52 (m, 4H), 2.48-2.19 (m, 4H), 2.02-1.58 (m, 8H), 1.58-1.38 (m, 3H), 1.38-1.12 (m, 1H), 1.06-0.97 (m, 3H), 0.97-0.74 (m, 3H), 0.71-0.66 (m, 1H), 0.66-0.31 (m, 3H). m/z: ES⁺ [M+H]⁺=601.35.

### Example 6.28 ((1S,5R)-6-(6-(2-(2-cyclopropyl-5-methyl-1H-imidazol-1-yl)-4-fluorophenoxy)-1,2,4-triazin-5-yl)-2,6-diazabicyclo[3.2.1]octan-2-yl)((1R,5R)-3-methyl-3-azabicyclo[3.1.0]hexan-1-yl)methanone and ((1S,5R)-6-(6-(2-(2-cyclopropyl-5-methyl-1H-imidazol-1-yl)-4-fluorophenoxy)-1,2,4-triazin-5-yl)-2,6-diazabicyclo[3.2.1]octan-2-yl)((1S,5S)-3-methyl-3-azabicyclo[3.1.0]hexan-1-yl)methanone

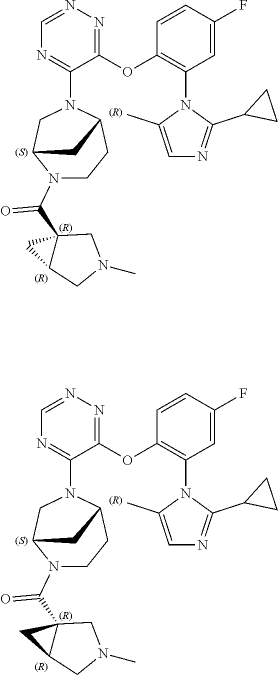

The reductive amination of the mixture of ((1R,5R)-3-azabicyclo[3.1.0]hexan-1-yl)((1S,5R)-6-(6-(2-(2-cyclopropyl-5-methyl-1H-imidazol-1-yl)-4-fluorophenoxy)-1,2,4-triazin-5-yl)-2,6-diazabicyclo[3.2.1]octan-2-yl)methanone and ((1S,5S)-3-azabicyclo[3.1.0]hexan-1-yl)((1S,5R)-6-(6-(2-(2-cyclopropyl-5-methyl-1H-imidazol-1-yl)-4-fluorophenoxy)-1,2,4-triazin-5-yl)-2,6-diazabicyclo[3.2.1]octan-2-yl)methanone (80 mg, 0.15 mmol) was performed according to general procedure H1. The residue was purified using method Prep-HPLC18 to afford the title mixture as a colourless solid (36 mg, 44%).

¹H NMR (400 MHz, DMSO-d₆, 300 K) δ 8.14-8.14 (s, 1H), 7.92-7.86 (m, 1H), 7.62-7.53 (m, 1H), 7.45-7.33 (m, 1H), 7.31-7.06 (m, 1H), 6.65-6.48 (m, 1H), 5.12-4.73 (m, 1H), 4.72-4.35 (m, 1H), 4.17-3.82 (m, 1H), 3.78-3.62 (m, 1H), 3.50-3.37 (m, 1H), 3.09-2.83 (m, 1H), 2.82-2.52 (m, 3H), 2.42-2.31 (m, 2H), 2.09-1.91 (m, 4H), 1.87-1.75 (m, 2H), 1.74-1.47 (m, 5H), 0.91-0.65 (m, 6H). m/z: ES⁺ [M+H]⁺=630.31.

### Example 6.29 ((1S,5R)-6-(5-(2-(2-cyclopropyl-5-methyl-1H-imidazol-1-yl)-4-fluorophenoxy)pyrimidin-4-yl)-2,6-diazabicyclo[3.2.1]octan-2-yl)((3aR,6aR)-2-methylhexahydrocyclopenta[c]pyrrol-3a(1H)-yl)methanone and ((1S,5R)-6-(5-(2-(2-cyclopropyl-5-methyl-1H-imidazol-1-yl)-4-fluorophenoxy)pyrimidin-4-yl)-2,6-diazabicyclo[3.2.1]octan-2-yl)((3aS,6aS)-2-methylhexahydrocyclopenta[c]pyrrol-3a(1H)-yl)methanone

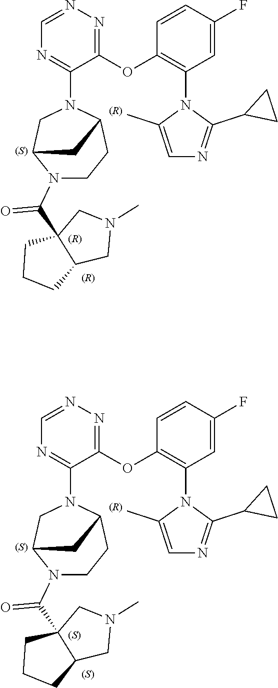

The reductive amination of the mixture of ((1S,5R)-6-(5-(2-(2-cyclopropyl-5-methyl-1H-imidazol-1-yl)-4-fluorophenoxy)pyrimidin-4-yl)-2,6-diazabicyclo[3.2.1]octan-2-yl)((3aR,6aR)-hexahydrocyclopenta[c]pyrrol-3a(1H)-yl)methanone and ((1S,5R)-6-(5-(2-(2-cyclopropyl-5-methyl-1H-imidazol-1-yl)-4-fluorophenoxy)pyrimidin-4-yl)-2,6-diazabicyclo[3.2.1]octan-2-yl)((3aS,6aS)-hexahydrocyclopenta[c]pyrrol-3a(1H)-yl)methanone (50 mg, 0.09 mmol) was performed according to general procedure H1. The residue was purified using method RPFC1 to afford the title mixture as a colourless solid (8 mg, 16%).

¹H NMR (400 MHz, DMSO-d₆, 300 K) δ 8.36-8.32 (m, 1H), 7.91-7.89 (m, 1H), 7.60-7.57 (m, 1H), 7.44-7.39 (m, 1H), 7.18 (s, 1H), 6.58-6.55 (m, 1H), 5.20-4.09 (m, 3H), 3.70 (s, 2H), 3.67-3.60 (m, 2H), 3.00 (s, 1H), 2.74-2.60 (m, 1H), 2.51-2.50 (m, 1H), 2.43-2.28 (m, 1H), 2.25-2.07 (m, 4H), 1.96-1.95 (m, 3H), 1.84 (s, 2H), 1.75-1.52 (m, 6H), 1.43-1.23 (m, 2H), 0.76-0.74 (m, 4H). m/z: ES⁺ [M+H]⁺=572.30.

### Example 6.30 ((1S,5R)-6-(5-(2-(2-cyclopropyl-5-methyl-1H-imidazol-1-yl)-4-fluorophenoxy)pyrimidin-4-yl)-2,6-diazabicyclo[3.2.1]octan-2-yl)(7-methyl-7-azabicyclo[2.2.1]heptan-1-yl)methanone

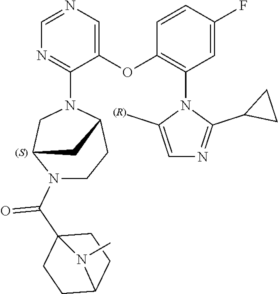

The reductive amination of (7-azabicyclo[2.2.1]heptan-1-yl)((1S,5R)-6-(5-(2-(2-cyclopropyl-5-methyl-1H-imidazol-1-yl)-4-fluorophenoxy)pyrimidin-4-yl)-2,6-diazabicyclo[3.2.1]octan-2-yl)methanone (70 mg, 0.13 mmol) was performed according to general procedure H1. The residue was purified using method Prep-HPLC1 to afford the title compound as a colourless solid (20 mg, 28%).

¹H NMR (400 MHz, DMSO-d₆, 300 K) δ 8.34 (s, 1H), 8.02-7.85 (m, 1H), 7.68-7.53 (m, 1H), 7.47-7.35 (m, 1H), 7.32-7.11 (m, 1H), 6.69-6.51 (m, 1H), 6.20-5.10 (m, 1H), 4.73-4.40 (m, 1H), 3.82-3.50 (m, 2H), 3.29-3.19 (m, 1H), 2.82-2.59 (m, 1H), 2.30-2.02 (m, 3H), 2.08-1.88 (m, 6H), 1.86-1.59 (m, 4H), 1.66-1.43 (m, 5H), 1.37-1.18 (m, 2H), 0.88-0.61 (m, 4H). m/z: ES⁺ [M+H]⁺=558.67.

### Example 7.01: N-ethyl-5-fluoro-N-isopropyl-2-((4-((1S,5R)-2-(2-methyl-2-(methylamino)propanoyl)-2,6-diazabicyclo[3.2.1]octan-6-yl)pyrimidin-5-yl)oxy)benzamide

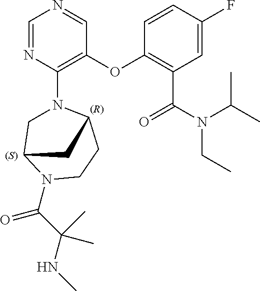

### Step 1: (9H-fluoren-9-yl)methyl (1-((1S,5R)-6-(5-(2-(ethyl(isopropyl)carbamoyl)-4-fluorophenoxy)pyrimidin-4-yl)-2,6-diazabicyclo[3.2.1]octan-2-yl)-2-methyl-1-oxopropan-2-yl)(methyl)carbamate

To a stirred solution of 2-((4-((1S,5R)-2,6-diazabicyclo[3.2.1]octan-6-yl)pyrimidin-5-yl)oxy)-N-ethyl-5-fluoro-N-isopropylbenzamide (120 mg, 0.29 mmol, 1.0 equiv) and 2-((((9H-fluoren-9-yl)methoxy) carbonyl)(methyl)amino)-2-methylpropanoic acid (98 mg, 0.29 mmol, 1.0 equiv) in DCM (2 mL) was treated with DIEA (113 mg, 0.87 mmol, 3.0 equiv) and CMPI (89 mg, 0.35 mmol, 1.2 equiv) at room temperature. The resulting mixture was stirred at room temperature for 3 h. The resulting mixture was diluted with water (20 mL) and was extracted with DCM (2×30 mL). The combined organic layers were washed with brine (2×10 mL), dried over sodium sulfate. After filtration, the filtrate was concentrated under reduced pressure. The residue was purified using method RFPC1 (10% to 100% gradient in 25 min), affording (9H-fluoren-9-yl)methyl (1-((1S,5R)-6-(5-(2-(ethyl(isopropyl)carbamoyl)-4-fluorophenoxy)pyrimidin-4-yl)-2,6-diazabicyclo[3.2.1]octan-2-yl)-2-methyl-1-oxopropan-2-yl)(methyl)carbamate (75 mg, 35%) as an off-white solid. m/z: ES⁺ [M+H]⁺=625.35.

### Step 2: N-ethyl-5-fluoro-N-isopropyl-2-((4-((1S,5R)-2-(2-methyl-2-(methylamino)propanoyl)-2,6-diazabicyclo[3.2.1]octan-6-yl)pyrimidin-5-yl)oxy)benzamide

A stirred solution of (9H-fluoren-9-yl)methyl (1-((1S,5R)-6-(5-(2-(ethyl(isopropyl)carbamoyl)-4-fluorophenoxy)pyrimidin-4-yl)-2,6-diazabicyclo[3.2.1]octan-2-yl)-2-methyl-1-oxopropan-2-yl)(methyl) carbamate (60 mg, 0.08 mmol, 1.0 equiv) in MeOH (1 mL) was treated with piperidine (14 mg, 0.16 mmol, 2.0 equiv) at room temperature for 2 h. The resulting mixture was concentrated under reduced pressure. The crude product was purified using method Prep-HPLC2 (Gradient: 48% B to 73% B in 10 min; RT1(min): 9.68). This resulted in N-ethyl-5-fluoro-N-isopropyl-2-((4-((1S,5R)-2-(2-methyl-2-(methylamino)propanoyl)-2,6-diazabicyclo[3.2.1]octan-6-yl)pyrimidin-5-yl)oxy)benzamide (8 mg, 19%) as a colourless solid.

¹H NMR (400 MHz, DMSO-d₆, 296 K) δ 8.34-8.31 (m, 1H), 7.83-7.76 (m, 1H), 7.31-7.23 (m, 2H), 6.99-6.93 (m, 1H), 6.60-5.00 (m, 1H), 4.82-4.55 (m, 1H), 4.46-4.04 (m, 1H), 3.81-3.68 (m, 3H), 3.26-3.15 (m, 2H), 2.34-2.08 (m, 3H), 2.09-1.57 (m, 5H), 1.20-1.06 (m, 17H). m/z: ES⁺ [M+H]⁺=513.29.

### Example 7.02: 2-((4-((1S,5R)-2-acetyl-2,6-diazabicyclo[3.2.1]octan-6-yl)pyrimidin-5-yl)oxy)-N-ethyl-5-fluoro-N-isopropylbenzamide

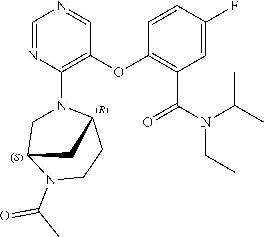

A solution of 2-((4-((1S,5R)-2,6-diazabicyclo[3.2.1]octan-6-yl)pyrimidin-5-yl)oxy)-N-ethyl-5-fluoro-N-isopropylbenzamide (100 mg, 0.24 mmol), acetic anhydride (49 mg, 0.48 mmol) and NEt₃ (122 mg, 1.2 mmol) in DCM (2 mL) was stirred at room temperature for 2 h under nitrogen. The resulting mixture was diluted with water (15 mL), and extracted with DCM (2×30 mL). The combined organic layers were washed with brine (2×20 mL), and dried over Na₂SO₄. After filtration, the filtrate was concentrated under reduced pressure, and the crude product was purified using method Prep-HPLC1 (Gradient: 18% B to 39% B in 7 min; RT1(min): 6.5) to afford 2-((4-((1S,5R)-2-acetyl-2,6-diazabicyclo[3.2.1]octan-6-yl)pyrimidin-5-yl)oxy)-N-ethyl-5-fluoro-N-isopropylbenzamide (60 mg, 54%) as a colourless solid.

¹H NMR (400 MHz, DMSO-d₆, 296 K) δ 8.40-8.25 (m, 1H), 7.90-7.61 (m, 1H), 7.39-7.16 (m, 2H), 7.18-6.65 (m, 1H), 5.21-4.98 (m, 3H), 4.90-4.28 (m, 2H), 4.21-3.74 (m, 2H), 3.70-3.57 (m, 1H), 3.56-3.45 (m, 1H), 3.28-3.18 (m, 1H), 3.17-3.05 (m, 1H), 2.05-1.99 (m, 1H), 1.95-1.81 (m, 4H), 1.79-1.52 (m, 2H), 1.19-1.16 (m, 1H), 1.15-1.09 (m, 4H), 1.08-0.98 (m, 4H). m/z: ES⁺ [M+H]⁺=456.20.

### Example 7.03: N-ethyl-5-fluoro-2-((4-((1S,5R)-2-(2-(3-hydroxyazetidin-1-yl)acetyl)-2,6-diazabicyclo[3.2.1]octan-6-yl)pyrimidin-5-yl)oxy)-N-isopropylbenzamide

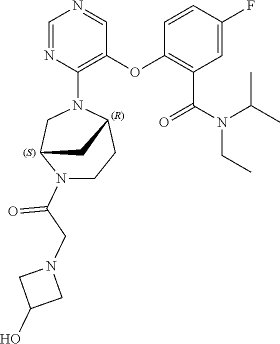

### Step 1: 2-((4-((1S,5R)-2-(2-Bromoacetyl)-2,6-diazabicyclo[3.2.1]octan-6-yl)pyrimidin-5-yl)oxy)-N-ethyl-5-fluoro-N-isopropylbenzamide

A mixture of 2-((4-((1S,5R)-2,6-diazabicyclo[3.2.1]octan-6-yl)pyrimidin-5-yl)oxy)-N-ethyl-5-fluoro-N-isopropylbenzamide (100 mg, 0.24 mmol, 1.0 equiv) and 2-bromoacetyl bromide (98 mg, 0.48 mmol, 2.0 equiv) and Na₂CO₃ (50 mg, 0.48 mmol, 2.0 eq) in DCM (2 mL) was stirred at room temperature for 2 h under air atmosphere. The crude resulting mixture was used in the next step directly without further purification. m/z: ES⁺ [M+H]⁺=534.43.

### Step 2: N-ethyl-5-fluoro-2-((4-((1S,5R)-2-(2-(3-hydroxyazetidin-1-yl)acetyl)-2,6-diazabicyclo[3.2.1]octan-6-yl)pyrimidin-5-yl)oxy)-N-isopropylbenzamide

A mixture of 2-((4-((1S,5R)-2-(2-bromoacetyl)-2,6-diazabicyclo[3.2.1]octan-6-yl)pyrimidin-5-yl)oxy)-N-ethyl-5-fluoro-N-isopropylbenzamide (70 mg, 0.13 mmol, 1.0 equiv), azetidin-3-ol (48 mg, 0.66 mmol, 5 equiv) and Na₂CO₃ (28 mg, 0.26 mmol, 2.0 equiv) in DCM (2 mL) was stirred at room temperature for 5 h under air atmosphere. The crude product (50 mg) was purified using method Prep-HPLC1 (Gradient: 11% B to 35% B in 7 min; RT1(min): 6.4) to afford N-ethyl-5-fluoro-2-((4-((1S,5R)-2-(2-(3-hydroxyazetidin-1-yl)acetyl)-2,6-diazabicyclo[3.2.1]octan-6-yl)pyrimidin-5-yl)oxy)-N-isopropylbenzamide (17 mg, 24%) as a colourless solid.

¹H NMR (400 MHz, DMSO-d₆, 297 K) δ 8.41-8.17 (m, 1H), 7.89-7.71 (m, 1H), 7.45-7.20 (m, 2H), 7.20-6.87 (m, 1H), 5.31-5.22 (m, 1H), 5.12-4.57 (m, 2H), 4.45-4.11 (m, 2H), 3.83-3.78 (m, 1H), 3.78-3.65 (m, 1H), 3.61-3.52 (m, 3H), 3.43-3.40 (m, 1H), 3.30-3.22 (m, 2H), 3.18-3.02 (m, 2H), 2.85-2.71 (m, 2H), 2.01-1.77 (m, 2H), 1.77-1.49 (m, 2H), 1.24-1.18 (m, 1H), 1.18-0.98 (m, 8H). m/z: ES⁺ [M+H]⁺=526.61.

### Example 7.04: 5-fluoro-N-isopropyl-2-((4-((1S,5R)-2-((S)-2-methylpyrrolidine-2-carbonyl)-2,6-diazabicyclo[3.2.1]octan-6-yl)pyrimidin-5-yl)oxy)benzamide

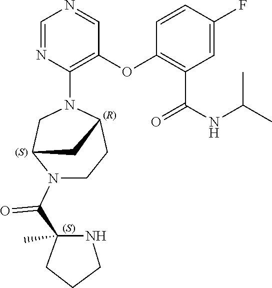

### Step 1: tert-butyl (S)-2-((1S,5R)-6-(5-(4-fluoro-2-(isopropylcarbamoyl)phenoxy)pyrimidin-4-yl)-2,6-diazabicyclo[3.2.1]octane-2-carbonyl)-2-methylpyrrolidine-1-carboxylate

2-((4-((1S,5R)-2-((S)-1-(tert-butoxycarbonyl)-2-methylpyrrolidine-2-carbonyl)-2,6-diaza bicyclo[3.2.1]octan-6-yl)pyrimidin-5-yl)oxy)-5-fluorobenzoic acid (80 mg, 0.14 mmol, 1.0 equiv) and i-PrNH₂ (18 mg, 0.31 mmol, 2.1 equiv) were coupled following general procedure A1. The resulting mixture was diluted with water (30 mL) and extracted with EtOAc (3×30 mL). The combined organic layers were washed with brine (30 mL), dried over Na₂SO₄, and concentrated under reduced pressure. The crude product was purified using method RPFC1 (Gradient: 40% B to 70% B in 10) to afford tert-butyl (S)-2-((1S,5R)-6-(5-(4-fluoro-2-(isopropylcarbamoyl)phenoxy)pyrimidin-4-yl)-2,6-diazabicyclo[3.2.1]octane-2-carbonyl)-2-methylpyrrolidine-1-carboxylate (80 mg, 93%) as a yellow solid. m/z: ES⁺ [M+H]⁺=597.30.

### Step 2: 5-fluoro-N-isopropyl-2-((4-((1S,5R)-2-((S)-2-methylpyrrolidine-2-carbonyl)-2,6-diazabicyclo[3.2.1]octan-6-yl)pyrimidin-5-yl)oxy)benzamide

The Boc deprotection of tert-butyl (S)-2-((1S,5R)-6-(5-(4-fluoro-2-(isopropylcarbamoyl)phenoxy)pyrimidin-4-yl)-2,6-diazabicyclo[3.2.1]octane-2-carbonyl)-2-methylpyrrolidine-1-carboxylate (490 mg, 0.979 mmol) was performed according to general procedure B1. The mixture was concentrated under reduced pressure and purified using method HPLC1 (Gradient: 32% B to 48% B in 7 min; RT1(min): 6.37) to afford 5-fluoro-N-isopropyl-2-((4-((1S,5R)-2-((S)-2-methylpyrrolidine-2-carbonyl)-2,6-diazabicyclo[3.2.1]octan-6-yl)pyrimidin-5-yl)oxy)benzamide (39 mg, 68%) as a colourless solid.

¹H NMR (400 MHz, DMSO-d₆, 297 K) δ 8.28 (s, 1H), 8.28-8.19 (m, 1H), 7.71 (s, 1H), 7.41-7.22 (m, 2H), 7.12-6.93 (m, 1H), 6.51-6.01 (m, 1H), 4.92-4.71 (m, 1H), 4.61-4.01 (m, 1H), 4.00-3.89 (m, 1H), 3.88-3.65 (m, 2H), 2.98-2.69 (m, 2H), 2.66-2.57 (m, 1H), 2.21-2.11 (m, 1H), 2.08-1.79 (m, 2H), 1.78-1.42 (m, 5H), 1.23 (s, 3H), 1.07-1.01 (m, 6H). m/z: ES⁺ [M+H]⁺=497.25.

### Example 7.05: N-ethyl-5-fluoro-N-isopropyl-2-((4-((1S,5R)-2-((3aR,6aS)-octahydrocyclopenta[b]pyrrole-3a-carbonyl)-2,6-diazabicyclo[3.2.1]octan-6-yl)pyrimidin-5-yl)oxy)benzamide and N-ethyl-5-fluoro-N-isopropyl-2-((4-((1S,5R)-2-((3aS,6aR)-octahydrocyclopenta[b]pyrrole-3a-carbonyl)-2,6-diazabicyclo[3.2.1]octan-6-yl)pyrimidin-5-yl)oxy)benzamide

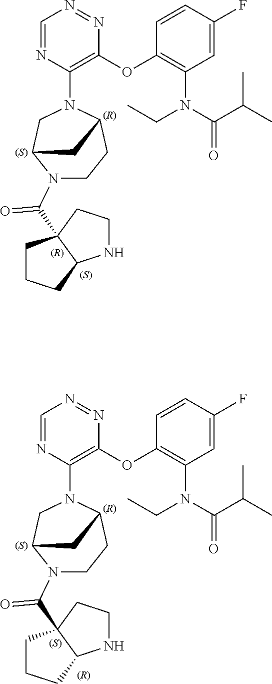

### Step 1: 2-((4-((1S,5R)-2-((3aRS,6aSR)-1-benzyloctahydrocyclopenta[b]pyrrole-3a-carbonyl)-2,6-diazabicyclo[3.2.1]octan-6-yl)pyrimidin-5-yl)oxy)-N-ethyl-5-fluoro-N-isopropylbenzamide

The coupling of rac-(3aR,6aS)-1-Benzylhexa-hydrocyclopenta[b]pyrrole-3a(1H)-carboxylic acid (84 mg, 0.34 mmol, 1.0 equiv) and 2-((4-((1S,5R)-2,6-diazabicyclo[3.2.1]octan-6-yl)pyrimidin-5-yl)oxy)-N-ethyl-5-fluoro-N-isopropyl-benzamide (154 mg, 0.34 mmol, 1.0 equiv) was performed according to general procedure A1. The residue was purified using method RPFC1 (10% to 100% gradient in 25 min) to afford 2-((4-((1S,5R)-2-((3aRS,6aSR)-1-benzyloctahydrocyclopenta[b]pyrrole-3a-carbonyl)-2,6-diazabicyclo[3.2.1]octan-6-yl)pyrimidin-5-yl)oxy)-N-ethyl-5-fluoro-N-isopropylbenzamide (100 mg, 46%) as a light yellow solid. m/z: ES⁺ [M+H]⁺=641.35.

### Step 2: N-ethyl-5-fluoro-N-isopropyl-2-((4-((1S,5R)-2-((3aR,6aS)-octahydrocyclopenta[b]pyrrole-3a-carbonyl)-2,6-diazabicyclo[3.2.1]octan-6-yl)pyrimidin-5-yl)oxy)benzamide and N-ethyl-5-fluoro-N-isopropyl-2-((4-((1S,5R)-2-((3aS,6aR)-octahydrocyclopenta[b]pyrrole-3a-carbonyl)-2,6-diazabicyclo[3.2.1]octan-6-yl)pyrimidin-5-yl)oxy)benzamide

To a solution of 2-((4-((1S,5R)-2-((3aRS,6aSR)-1-benzyloctahydrocyclopenta[b]pyrrole-3a-carbonyl)-2,6-diazabicyclo[3.2.1]octan-6-yl)pyrimidin-5-yl)oxy)-N-ethyl-5-fluoro-N-isopropylbenzamide (80 mg, 0.13 mmol, 1.0 equiv) in EtOAc (2 mL) was added Pd/C (10% wt, 100 mg, wet) under nitrogen. The mixture was stirred at room temperature for 9 h under hydrogen, and then filtered through a Celite pad and concentrated under reduced pressure. The crude product was purified using method prep-HPLC1 (Gradient: 21% B to 35% B in 7 min; Rt=7.4 min) to afford the mixture of N-ethyl-5-fluoro-N-isopropyl-2-((4-((1S,5R)-2-((3aR,6aS)-octahydrocyclopenta[b]pyrrole-3a-carbonyl)-2,6-diazabicyclo[3.2.1]octan-6-yl)pyrimidin-5-yl)oxy)benzamide and N-ethyl-5-fluoro-N-isopropyl-2-((4-((1S,5R)-2-((3aS,6aR)-octahydrocyclopenta[b]pyrrole-3a-carbonyl)-2,6-diazabicyclo[3.2.1]octan-6-yl)pyrimidin-5-yl)oxy)benzamide (17 mg, 24%) as a colourless solid.

¹H NMR (400 MHz, DMSO-d₆, 300 K) δ 8.32-8.31 (m, 1H), 7.84-7.76 (m, 1H), 7.33-7.22 (m, 2H), 7.00 (s, 1H), 5.22-4.55 (m, 2H), 4.50-3.77 (m, 5H), 3.41-3.37 (m, 1H), 3.26-3.12 (m, 2H), 2.82-2.80 (m, 1H), 2.64-2.61 (m, 1H), 2.07-1.59 (m, 11H), 1.40 (m, 2H), 1.15-0.98 (m, 9H). m/z: ES⁺ [M+H]⁺=551.30.

### Example 7.06: N-ethyl-5-fluoro-N-isopropyl-2-((4-((1S,5R)-2-(octahydropyrrolo[3,4-c]pyrrole-3a-carbonyl)-2,6-diazabicyclo[3.2.1]octan-6-yl)pyrimidin-5-yl)oxy)benzamide

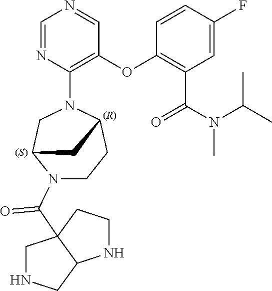

A solution of (3aS,6aS)-tert-butyl 6a-((1S,5R)-6-(5-(2-(ethyl(isopropyl)carbamoyl)-4-fluorophenoxy)pyrimidin-4-yl)-2,6-diaza-bicyclo[3.2.1]octane-2-carbonyl)-hexahydropyrrolo[3,4-c]pyrrole-2(1H)-carboxylate (70 mg, 0.11 mmol, 1.0 equiv) in HCl in dioxane (4 M, 3.0 mL) was stirred at room temperature for 1h. The mixture was basified to pH 9 with sat. NaHCO₃ and the resulting mixture was extracted with EtOAc (2×30 mL). The combined organic layers were washed with brine (60 mL), dried over Na₂SO₄, and concentrated under reduced pressure. The crude product purified by Prep-HPLC1 (12% B to 34% B in 8 min; Rt=7.68) to afford N-ethyl-5-fluoro-N-isopropyl-2-((4-((1S,5R)-2-(octahydropyrrolo[3,4-c]pyrrole-3a-carbonyl)-2,6-diazabicyclo[3.2.1]octan-6-yl)pyrimidin-5-yl)oxy)benzamide (19 mg, 33%/oyield) as a colourless solid.

¹HNMR (400 MHz, DMSO-d₆, 300K) δ 8.47-8.24 (m, 1H), 7.79-7.75 (m, 1H), 7.27-7.23 (m, 2H), 7.02-6.89 (m, 1H), 4.97-4.84 (m, 1H), 4.65-4.38 (m, 1H), 4.27-4.16 (m, 3H), 3.78-3.75 (m, 3H), 3.54-3.49 (m, 2H), 3.43-3.36 (m, 1H), 3.31-3.25 (m, 1H), 3.24-3.09 (m, 2H), 2.79-2.67 (m, 3H), 2.46-2.32 (m, 1H), 1.98-1.88 (m, 2H), 1.79-1.48 (m, 2H), 1.29-0.94 (m, 9H). 0.06-0.01 (m, 2H). m/z: ES⁺ [M+H]⁺=552.25.

### Example 7.07 and 7.08: N-ethyl-5-fluoro-N-isopropyl-2-((4-((1S,5R)-2-((3aR,7aR)-octahydro-1H-isoindole-3a-carbonyl)-2,6-diazabicyclo[3.2.1]octan-6-yl)pyrimidin-5-yl)oxy)benzamide and N-ethyl-5-fluoro-N-isopropyl-2-((4-((1S,5R)-2-((3aS,7aS)-octahydro-1H-isoindole-3a-carbonyl)-2,6-diazabicyclo[3.2.1]octan-6-yl)pyrimidin-5-yl)oxy)benzamide

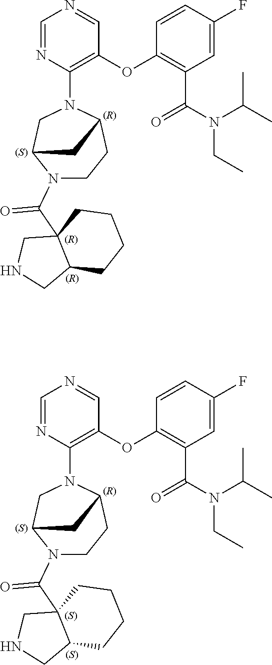

### Step 1: 2-(4-((1S,5R)-2-((3aRS,7aRS)-2-benzyl-octahydro-1H-isoindole-3a-carbonyl)-2,6-diaza-bicyclo[3.2.1]octan-6-yl)pyrimidin-5-yloxy)-N-ethyl-5-fluoro-N-isopropylbenzamide

2-((4-((1S,5R)-2,6-diazabicyclo[3.2.1]octan-6-yl)pyrimidin-5-yl)oxy)-N-ethyl-5-fluoro-N-isopropylbenzamide hydrochloride (250 mg, 0.56 mmol, 1.0 equiv) and rac-(3aR,7aR)-2-benzyl-octahydro-3aH-isoindole-3a-carboxylic acid (160 mg, 0.61 mmol, 1.1 equiv) were coupled according to general procedure A1. The residue was purified by RPFC1 (30% to 80% gradient in 20 min) to afford 2-(4-((1S,5R)-2-((3aRS,7aRS)-2-benzyl-octahydro-1H-isoindole-3a-carbonyl)-2,6-diaza-bicyclo[3.2.1]octan-6-yl)pyrimidin-5-yloxy)-N-ethyl-5-fluoro-N-isopropylbenzamide (270 mg, 74%) as a colourless solid. m/z: ES⁺ [M+H]⁺=655.10.

### Step 2: N-ethyl-5-fluoro-N-isopropyl-2-((4-((1S,5R)-2-((3aR,7aR)-octahydro-1H-isoindole-3a-carbonyl)-2,6-diazabicyclo[3.2.1]octan-6-yl)pyrimidin-5-yl)oxy)benzamide and N-ethyl-5-fluoro-N-isopropyl-2-((4-((1S,5R)-2-((3aS,7aS)-octahydro-1H-isoindole-3a-carbonyl)-2,6-diazabicyclo[3.2.1]octan-6-yl)pyrimidin-5-yl)oxy)benzamide

A solution of 2-(4-((1S,5R)-2-((3aRS,7aRS)-2-benzyl-octahydro-1H-isoindole-3a-carbonyl)-2,6-diazabicyclo[3.2.1]octan-6-yl)pyrimidin-5-yloxy)-N-ethyl-5-fluoro-N-isopropylbenzamide (160 mg, 0.24 mmol, 1.0 equiv) and Pd/C (100 mg, 10% wt, wet) in MeOH (1.0 mL) was stirred at room temperature for 8 h under hydrogen. The mixture was then filtered through a Celite pad and concentrated under reduced pressure. The crude product was purified by Prep-HPLC6 (Gradient 6% B to 22% B in 5 min; Rt=4.93 min) to afford N-ethyl-5-fluoro-N-isopropyl-2-((4-((1S,5R)-2-((3aR,7aR)-octahydro-1H-isoindole-3a-carbonyl)-2,6-diazabicyclo[3.2.1]octan-6-yl)pyrimidin-5-yl)oxy)benzamide and N-ethyl-5-fluoro-N-isopropyl-2-((4-((1S,5R)-2-((3aS,7aS)-octahydro-1H-isoindole-3a-carbonyl)-2,6-diazabicyclo[3.2.1]octan-6-yl)pyrimidin-5-yl)oxy)benzamide (formate salt, 33 mg, 22%) as a colourless solid. m/z: ES⁺ [M+H]⁺=565.35.

### Step 3: N-ethyl-5-fluoro-N-isopropyl-2-((4-((1S,5R)-2-((3aR,7aR)-octahydro-1H-isoindole-3a-carbonyl)-2,6-diazabicyclo[3.2.1]octan-6-yl)pyrimidin-5-yl)oxy)benzamide and N-ethyl-5-fluoro-N-isopropyl-2-((4-((1S,5R)-2-((3aS,7aS)-octahydro-1H-isoindole-3a-carbonyl)-2,6-diazabicyclo[3.2.1]octan-6-yl)pyrimidin-5-yl)oxy)benzamide

Separation of a 100 mg mixture of N-ethyl-5-fluoro-N-isopropyl-2-((4-((1S,5R)-2-((3aR,7aR)-octahydro-1H-isoindole-3a-carbonyl)-2,6-diazabicyclo[3.2.1]octan-6-yl)pyrimidin-5-yl)oxy)benzamide and N-ethyl-5-fluoro-N-isopropyl-2-((4-((1S,5R)-2-((3aS,7aS)-octahydro-1H-isoindole-3a-carbonyl)-2,6-diazabicyclo[3.2.1]octan-6-yl)pyrimidin-5-yl)oxy)benzamide by CHIRALPAKIF (Mobile Phase A: Hex(0.2% IPAmine), Mobile Phase B: EtOH:DCM=1:1; Isocratic Gradient 40% B for 18 min) gave two single stereoisomers:

Peak #1: Rt=10.18 min, colourless solid (22 mg, 22%).

¹HNMR (400 MHz, DMSO-d6, 300K) δ 8.43-8.24 (m, 1H), 7.90-7.71 (m, 1H), 7.34-7.19 (m, 2H), 7.08-6.90 (m, 1H), 5.26-4.26 (m, 3H), 3.98-3.64 (m, 3H), 3.59-3.50 (m, 1H), 3.44-3.32 (m, 1H), 3.30-3.09 (m, 2H), 3.07-2.88 (m, 2H), 2.80-2.64 (m, 2H), 2.46-2.36 (m, 1H), 2.03-1.67 (m, 4H), 1.65-1.22 (m, 7H), 1.22-0.80 (m, 10H). m/z: ES⁺ [M+H]⁺=565.35.

Peak #2: Rt=12.86 min, colourless solid (35 mg, 35%).

¹H NMR (400 MHz, DMSO-d₆, 300K) δ 8.43-8.24 (m, 1H), 7.90-7.71 (m, 1H), 7.34-7.19 (m, 2H), 7.08-6.90 (m, 1H), 5.26-4.26 (m, 3H), 3.98-3.64 (m, 3H), 3.59-3.50 (m, 1H), 3.44-3.32 (m, 1H), 3.30-3.09 (m, 2H), 3.07-2.88 (m, 2H), 2.80-2.64 (m, 2H), 2.46-2.36 (m, 1H), 2.03-1.67 (m, 4H), 1.65-1.22 (m, 7H), 1.22-0.80 (m, 10H). m/z: ES⁺ [M+H]⁺=565.35.

### Example 7.09 and 7.10: N-ethyl-5-fluoro-2-((4-((1S,5R)-2-((3aR,6aR)-hexahydro-1H-furo[3,4-c]pyrrole-3a-carbonyl)-2,6-diazabicyclo[3.2.1]octan-6-yl)pyrimidin-5-yl)oxy)-N-isopropylbenzamide and N-ethyl-5-fluoro-2-((4-((1S,5R)-2-((3aS,6aS)-hexahydro-1H-furo[3,4-c]pyrrole-3a-carbonyl)-2,6-diazabicyclo[3.2.1]octan-6-yl)pyrimidin-5-yl)oxy)-N-isopropylbenzamide

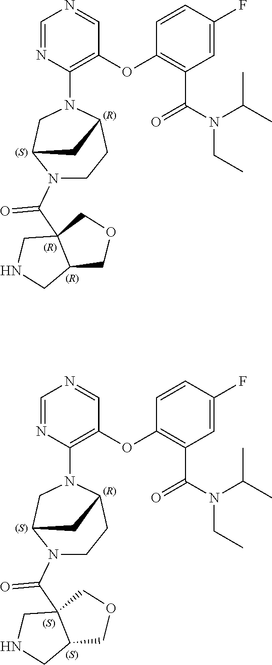

### Step 1: N-ethyl-5-fluoro-2-((4-((1S,5R)-2-((3aR,6aR)-hexahydro-1H-furo[3,4-c]pyrrole-3a-carbonyl)-2,6-diazabicyclo[3.2.1]octan-6-yl)pyrimidin-5-yl)oxy)-N-isopropylbenzamide and N-ethyl-5-fluoro-2-((4-((1S,5R)-2-((3aS,6aS)-hexahydro-1H-furo[3,4-c]pyrrole-3a-carbonyl)-2,6-diazabicyclo[3.2.1]octan-6-yl)pyrimidin-5-yl)oxy)-N-isopropylbenzamide

A solution of 2-((4-((1S,5R)-2-((3aRS,6aRS)-5-benzylhexahydro-1H-furo[3,4-c]pyrrole-3a-carbonyl)-2,6-diazabicyclo[3.2.1]octan-6-yl)pyrimidin-5-yl)oxy)-N-ethyl-5-fluoro-N-isopropylbenzamide (100 mg, 0.16 mmol, 1.0 equiv) and Pd/C (10% wt, 80 mg, wet) in 25 mL MeOH were stirred at room temperature for 4 h under hydrogen. The mixture was then filtered through a pad of Celite and concentrated under reduced pressure. The crude product was purified by Prep-HPLC1 (Gradient 21% B to 33% B in 7 min; Rt=7.55) to afford N-ethyl-5-fluoro-2-((4-((1S,5R)-2-((3aR,6aR)-hexahydro-1H-furo[3,4-c]pyrrole-3a-carbonyl)-2,6-diazabicyclo[3.2.1]octan-6-yl)pyrimidin-5-yl)oxy)-N-isopropylbenzamide and N-ethyl-5-fluoro-2-((4-((1S,5R)-2-((3aS,6aS)-hexahydro-1H-furo[3,4-c]pyrrole-3a-carbonyl)-2,6-diazabicyclo[3.2.1]octan-6-yl)pyrimidin-5-yl)oxy)-N-isopropylbenzamide (16 mg, 19%) as an off-white solid. m/z: ES⁺ [M+H]⁺=553.25.

### Step 2: N-ethyl-5-fluoro-2-((4-((1S,5R)-2-((3aR,6aR)-hexahydro-1H-furo[3,4-c]pyrrole-3a-carbonyl)-2,6-diazabicyclo[3.2.1]octan-6-yl)pyrimidin-5-yl)oxy)-N-isopropylbenzamide and N-ethyl-5-fluoro-2-((4-((1S,5R)-2-((3aS,6aS)-hexahydro-1H-furo[3,4-c]pyrrole-3a-carbonyl)-2,6-diazabicyclo[3.2.1]octan-6-yl)pyrimidin-5-yl)oxy)-N-isopropylbenzamide

Separation of a 100 mg mixture of of N-ethyl-5-fluoro-2-((4-((1S,5R)-2-((3aR,6aR)-hexahydro-1H-furo[3,4-c]pyrrole-3a-carbonyl)-2,6-diazabicyclo[3.2.1]octan-6-yl)pyrimidin-5-yl)oxy)-N-isopropylbenzamide and N-ethyl-5-fluoro-2-((4-((1S,5R)-2-((3aS,6aS)-hexahydro-1H-furo[3,4-c]pyrrole-3a-carbonyl)-2,6-diazabicyclo[3.2.1]octan-6-yl)pyrimidin-5-yl)oxy)-N-isopropylbenzamide was achieved by CHIRALART2 (isocratic 50% EtOH/DCM in 14.5 min) to afford:

Peak #1: Rt=7.69 min, colourless solid (18 mg, 18%)

¹H NMR (400 MHz, DMSO-d₆, 300 K) δ 8.36-8.29 (m, 1H), 7.95-7.83 (m, 1H), 7.35-7.02 (m, 3H), 5.11-4.82 (m, 1H), 4.80-4.05 (m, 2H), 4.02-3.75 (m, 5H), 3.59-3.42 (m, 4H), 3.25-3.13 (m, 3H), 3.05-2.90 (m, 1H), 2.80-2.52 (m, 3H), 2.12-1.87 (m, 2H), 1.82-1.52 (m, 2H), 1.19-0.96 (m, 9H). m/z: ES⁺ [M+H]⁺=553.25.

Peak #2: Rt=12.06 min, colourless solid (27 mg, 27%) ¹H NMR (400 MHz, DMSO-d₆, 300 K) δ 8.36-8.29 (m, 1H), 7.95-7.83 (m, 1H), 7.35-7.02 (m, 3H), 5.11-4.82 (m, 1H), 4.80-4.05 (m, 2H), 4.02-3.75 (m, 6H), 3.59-3.42 (m, 3H), 3.25-3.13 (m, 4H), 3.05-2.52 (m, 3H), 2.12-1.87 (m, 2H), 1.82-1.52 (m, 2H), 1.19-0.96 (m, 9H). m/z: ES⁺ [M+H]⁺=553.30.

### Example 7.11 N-(5-fluoro-2-((5-((1S,5R)-2-((3aR,6aR)-octahydrocyclopenta[c]pyrrole-3a-carbonyl)-2,6-diazabicyclo[3.2.1]octan-6-yl)-1,2,4-triazin-6-yl)oxy)phenyl)isobutyramide

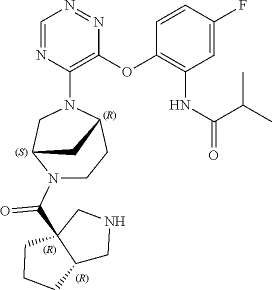

### Step 1: tert-butyl (3aR,6aR)-3a-((1S,5R)-6-(6-(2-(N-benzylisobutyramido)-4-fluorophenoxy)-3-chloro-1,2,4-triazin-5-yl)-2,6-diazabicyclo[3.2.1]octane-2-carbonyl)hexahydrocyclopenta[c]pyrrole-2(1H)-carboxylate

N-(2-((5-((1S,5R)-2,6-diazabicyclo[3.2.1]octan-6-yl)-3-chloro-1,2,4-triazin-6-yl)oxy)-5-fluorophenyl)-N-benzylisobutyramide (200 mg, 0.39 mmol, 1.0 equiv) and (3aR,6aR)-2-(tert-butoxycarbonyl)hexahydrocyclopenta[c]pyrrole-3a(1H)-carboxylic acid (109 mg, 0.43 mmol, 1.1 equiv) were coupled according to general procedure A1. The resulting mixture was diluted with water (50 mL) and extracted with EtOAc (2×50 mL). The combined organic layers were washed with water (2×50 mL), dried over Na₂SO₄, and concentrated under reduced pressure. The residue was purified using method RPFC1 (10% to 100% gradient in 20 min) to afford tert-butyl (3aR,6aR)-3a-((1S,5R)-6-(6-(2-(N-benzylisobutyramido)-4-fluorophenoxy)-3-chloro-1,2,4-triazin-5-yl)-2,6-diazabicyclo[3.2.1]octane-2-carbonyl)hexahydrocyclopenta[c]pyrrole-2(1H)-carboxylate (220 mg, 75%) as a yellow solid. m/z: ES⁺ [M+H]⁺=624.15.

### Step 2: N-(5-fluoro-2-((5-((1S,5R)-2-((3aR,6aR)-octahydrocyclopenta[c]pyrrole-3a-carbonyl)-2,6-diazabicyclo[3.2.1]octan-6-yl)-1,2,4-triazin-6-yl)oxy)phenyl)isobutyramide

A suspension of tert-butyl (3aR,6aR)-3a-((1S,5R)-6-(6-(2-(N-benzylisobutyramido)-4-fluorophenoxy)-3-chloro-1,2,4-triazin-5-yl)-2,6-diazabicyclo[3.2.1]octane-2-carbonyl)hexahydrocyclopenta[c]pyrrole-2(1H)-carboxylate (180 mg, 0.24 mmol, 1.0 equiv) and Pd/C (10% wt, 26 mg, wet) in acetic acid (15 mL) and HCl (4.0 M in EtOAc, 5 mL) was hydrogenated at 80° C. for 2 h. The mixture was then cooled to room temperature, filtered through a Celite pad and concentrated under reduced pressure. The residue was purified using method Prep-HPLC6 (Gradient 3% B to 27% B in 7 min; Rt=6.2 min) to afford N-(5-fluoro-2-((5-((1S,5R)-2-((3aR,6aR)-octahydrocyclopenta[c]pyrrole-3a-carbonyl)-2,6-diazabicyclo[3.2.1]octan-6-yl)-1,2,4-triazin-6-yl)oxy)phenyl)isobutyramide (14 mg, 10%) as an off-white solid.

¹H NMR (400 MHz, DMSO-d₆, 300 K) δ 293.7 K) δ 9.55-9.35 (m, 1H), 8.51-8.35 (m, 1H), 8.29 (s, 1H), 7.90-7.75 (m, 1H), 7.40-7.30 (m, 1H), 7.10-7.00 (m, 1H), 5.32-5.10 (m, 1H), 4.95-4.63 (m, 1H), 4.35-3.88 (m, 4H), 3.62-2.58 (m, 6H), 2.04-1.68 (m, 9H), 1.47-1.30 (m, 2H), 1.09-0.92 (m, 6H). m/z: ES⁺ [M+H]⁺=524.25.

### Example 8. Menin-KMT2Apeptide Inhibition Assays

Proteins: Wild-type and M327B

Compound-mediated disruption of the interaction between menin protein and a KMT2A peptide was measured using a Fluorescence Polarisation assay. Assay buffer (Tris pH 8.0, 50 mM, NaC2 120 mM, CA-630 0.005, DTT 2 mM) was prepared and, along with test compounds in DMSO, was transferred to a 384-well assay plate. The final DMSO concentration was 100 (v/v). Menin protein (wild-type or M327I mutant) solution was added to a final concentration of 4 nM. The assay plate was incubated for 30 min at 25° C. KMT2A peptide solution was then added to a final concentration of 1 nM and the assay plate was incubated for 60 min at 25° C. Assay plates were read on a BMG plate reader (460-520). The results are shown in Table B; key: A: <0.1 μM; B: <1 μM; C: <10 μM; D: >10 μM.

TABLE B
Fluorescence polarization (FP) data.

| | WT | M327I |
|---|---|---|
| Example | Menin | Menin |
| 1.001 | A | B |
| 1.002 | A | B |
| 1.003 | B | C |
| 1.004 | B | B |
| 1.005 | B | C |
| 1.006 | C | D |
| 1.007 | C | C |
| 1.008 | B | C |
| 1.009 | A | B |
| 1.010 | B | B |
| 1.011 | A | A |
| 1.012 | B | C |
| 1.013 | A | A |
| 1.014 | A | B |
| 1.015 | A | B |
| 1.016 | A | B |
| 1.017 | B | B |
| 1.018 | B | C |
| 1.019 | B | C |
| 1.020 | A | C |
| 1.021 | A | B |
| 1.022 | B | C |
| 1.023 | B | C |
| 1.024 | B | C |
| 1.025 | A | C |
| 1.026 | A | B |
| 1.027 | B | C |
| 1.028 | A | C |
| 1.029 | A | A |
| 1.030 | A | B |
| 1.031 | A | A |
| 1.032 | A | A |
| 1.033 | A | C |
| 1.034 | A | C |
| 1.035 | B | C |
| 1.036 | A | A |
| 1.037 | A | B |
| 1.038 | A | B |
| 1.039 | A | A |
| 1.040 | A | B |
| 1.041 | A | B |
| 1.042 | A | B |
| 1.043 | A | A |
| 1.044 | A | A |
| 1.045 | B | B |
| 1.046 | B | B |
| 1.047 | B | B |
| 1.048 | A | B |
| 1.049 | B | C |
| 1.050 | A | B |
| 1.051 | A | A |
| 1.052 | B | C |
| 1.053 | B | C |
| 1.054 | B | D |
| 1.055 | C | D |
| 1.056 | B | C |
| 1.057 | B | C |
| 1.058 | B | C |
| 1.059 | C | D |
| 1.060 | C | D |
| 1.061 | A | C |
| 1.062 | A | B |
| 1.063 | A | A |
| 1.064 | C | D |
| 1.065 | B | C |
| 1.066 | A | A |
| 1.067 | A | B |
| 1.068 | A | A |
| 1.069 | B | B |
| 1.070 | A | A |
| 1.071 | A | B |
| 1.072 | A | B |
| 1.073 | A | B |
| 1.074 | A | B |
| 1.075 | A | B |
| 1.076 | A | B |
| 1.077 | A | A |
| 1.078 | A | C |
| 1.079 | B | B |
| 1.080 | A | A |
| 1.081 | B | C |
| 1.082 | A | B |
| 1.083 | A | B |
| 1.084 | A | A |
| 1.085 | C | C |
| 1.086 | A | B |
| 1.087 | B | C |
| 1.088 | A | B |
| 1.089 | A | A |
| 1.090 | B | B |
| 1.091 | B | C |
| 1.092 | A | A |
| 1.093 | A | B |
| 1.094 | A | B |
| 1.095 | B | B |
| 1.096 | B | C |
| 1.097 | B | C |
| 1.098 | A | B |
| 1.099 | A | B |
| 1.100 | A | B |
| 1.101 | B | C |
| 1.102 | B | C |
| 1.103 | B | B |
| 1.104 | A | A |
| 1.105 | B | C |
| 1.106 | A | A |
| 1.107 | A | A |
| 1.108 | A | C |
| 1.109 | A | B |
| 1.110 | A | B |
| 1.111 | A | B |
| 1.112 | A | B |
| 1.113 | A | A |
| 1.114 | A | B |
| 1.115 | A | B |
| 1.116 | A | B |
| 1.117 | B | C |
| 1.118 | B | C |
| 1.119 | A | B |
| 1.120 | B | B |
| 1.121 | B | C |
| 1.122 | A | B |
| 1.123 | A | B |
| 1.124 | B | D |
| 1.125 | C | D |
| 1.126 | B | C |
| 1.127 | B | D |
| 1.128 | B | D |
| 1.129 | C | D |
| 1.130 | C | D |
| 1.131 | C | D |
| 1.132 | C | D |
| 1.133 | D | D |
| 1.134 | C | D |
| 1.135 | C | D |
| 1.136 | B | C |
| 1.137 | B | C |
| 1.138 | C | D |
| 1.139 | B | D |
| 1.140 | D | D |
| 1.141 | C | C |
| 1.142 | C | D |
| 1.143 | A | B |
| 1.144 | A | B |
| 1.145 | A | B |
| 1.146 | A | B |
| 1.147 | A | A |
| 1.148 | A | A |
| 1.149 | A | A |
| 1.150 | A | B |
| 1.151 | A | A |
| 1.152 | B | C |
| 1.153 | A | A |
| 1.154 | A | A |
| 1.155 | B | C |
| 2.01 | A | A |
| 2.02 | A | A |
| 2.03 | A | A |
| 2.04 | A | B |
| 2.05 | A | A |
| 2.06 | A | A |
| 2.07 | A | A |
| 2.08 | A | B |
| 2.09 | A | A |
| 2.10 | A | A |
| 2.11 | A | A |
| 2.12 | A | A |
| 2.13 | A | A |
| 2.14 | A | A |
| 2.15 | A | A |
| 2.16 | A | A |
| 2.17 | A | A |
| 2.18 | A | A |
| 2.19 | A | A |
| 2.20 | A | A |
| 2.21 | A | A |
| 2.22 | B | B |
| 2.23 | A | A |
| 2.24 | A | A |
| 2.25 | A | A |
| 2.26 | A | A |
| 2.27 | A | A |
| 2.28 | A | A |
| 2.29 | A | A |
| 2.30 | A | A |
| 2.31 | A | A |
| 2.32 | A | A |
| 2.33 | A | B |
| 2.34 | A | A |
| 2.35 | A | A |
| 2.36 | A | A |
| 3.01 | A | B |
| 3.02 | A | B |
| 3.03 | A | B |
| 3.04 | A | A |
| 3.05 | A | A |
| 3.06 | A | A |
| 3.07 | A | A |
| 3.08 | A | A |
| 3.09 | A | A |
| 3.10 | A | A |
| 3.11 | A | A |
| 3.12 | A | A |
| 3.13 | A | A |
| 3.14 | A | B |
| 3.15 | A | A |
| 3.16 | A | A |
| 3.17 | A | A |
| 3.18 | A | A |
| 3.19 | A | A |
| 3.20 | A | A |
| 3.21 | A | A |
| 3.22 | A | A |
| 3.22 | A | A |
| 3.23 | A | A |
| 3.25 | A | A |
| 3.26 | B | C |
| 3.27 | B | B |
| 3.28 | A | A |
| 3.29 | A | A |
| 3.30 | A | A |
| 3.31 | A | A |
| 3.32 | A | A |
| 3.33 | A | A |
| 3.34 | A | A |
| 3.35 | A | A |
| 3.36 | A | A |
| 3.37 | A | A |
| 3.38 | A | A |
| 3.40 | A | A |
| 3.41 | A | A |
| 3.42 | A | A |
| 3.43 | A | A |
| 3.44 | A | A |
| 3.45 | A | A |
| 3.46 | A | A |
| 3.47 | A | A |
| 3.48 | A | A |
| 3.49 | A | A |
| 3.50 | A | A |
| 3.51 | A | A |
| 3.52 | A | A |
| 3.53 | A | A |
| 3.54 | A | A |
| 3.55 | A | A |
| 3.56 | A | A |
| 3.57 | A | A |
| 3.58 | A | A |
| 3.59 | A | A |
| 3.60 | A | A |
| 3.61 | A | A |
| 3.62 | B | C |
| 3.63 | A | A |
| 3.64 | A | A |
| 3.65 | A | A |
| 3.66 | A | A |
| 3.67 | A | A |
| 3.68 | A | A |
| 3.69 | A | A |
| 3.70 | A | A |
| 3.71 | A | A |
| 3.72 | A | A |
| 3.73 | A | A |
| 3.74 | A | A |
| 3.75 | A | B |
| 3.76 | A | A |
| 3.77 | A | A |
| 3.78 | A | A |
| 3.79 | A | A |
| 3.80 | A | B |
| 3.81 | A | A |
| 3.82 | A | A |
| 3.83 | A | A |
| 3.84 | A | A |
| 3.85 | A | A |
| 3.86 | A | A |
| 3.87 | A | A |
| 3.88 | A | A |
| 4.01 | A | A |
| 4.02 | A | A |
| 4.03 | A | A |
| 4.04 | A | A |
| 5.01 | A | A |
| 5.02 | A | A |
| 5.03 | A | A |
| 5.04 | A | B |
| 5.05 | B | B |
| 5.06 | A | A |
| 5.07 | A | A |
| 5.08 | A | A |
| 5.09 | A | A |
| 5.10 | A | A |
| 5.11 | A | A |
| 5.12 | A | B |
| 5.13 | A | A |
| 5.14 | A | B |
| 5.15 | A | A |
| 5.16 | A | B |
| 5.17 | A | B |
| 5.18 | A | A |
| 5.19 | A | A |
| 5.20 | B | C |
| 5.21 | A | A |
| 5.22 | A | A |
| 5.23 | A | A |
| 5.24 | A | A |
| 5.25 | A | B |
| 5.26 | A | A |
| 5.27 | A | A |
| 5.28 | A | A |
| 5.29 | A | A |
| 5.30 | A | A |
| 5.31 | A | A |
| 5.32 | A | A |
| 5.33 | A | A |
| 5.34 | A | A |
| 5.35 | A | A |
| 5.36 | A | A |
| 5.37 | A | A |
| 5.38 | A | A |
| 5.39 | A | A |
| 5.40 | A | A |
| 5.41 | A | A |
| 5.42 | A | A |
| 5.43 | A | A |
| 5.44 | A | A |
| 5.45 | A | A |
| 5.46 | A | A |
| 5.47 | A | A |
| 5.48 | A | A |
| 5.49 | A | A |
| 5.50 | A | A |
| 5.51 | A | A |
| 5.52 | A | A |
| 5.53 | A | A |
| 5.54 | B | C |
| 5.55 | A | A |
| 5.56 | A | A |
| 5.57 | A | A |
| 5.58 | A | A |
| 5.59 | A | A |
| 5.60 | A | A |
| 5.61 | A | A |
| 5.62 | B | C |
| 5.63 | A | A |
| 5.64 | A | A |
| 5.65 | A | A |
| 5.66 | A | A |
| 5.67 | A | A |
| 5.68 | A | A |
| 5.69 | A | A |
| 5.70 | C | C |
| 5.71 | A | B |
| 5.72 | A | A |
| 5.73 | A | A |
| 5.74 | A | A |
| 5.75 | A | A |
| 5.76 | A | A |
| 5.77 | A | A |
| 5.78 | A | A |
| 5.79 | A | A |
| 5.80 | A | A |
| 5.81 | A | A |
| 5.82 | A | A |
| 5.83 | A | A |
| 5.84 | A | B |
| 5.85 | A | A |
| 5.86 | A | A |
| 5.87 | A | A |
| 5.88 | A | A |
| 5.89 | A | A |
| 5.90 | A | A |
| 5.91 | A | A |
| 5.92 | A | A |
| 5.93 | A | A |
| 5.94 | A | A |
| 5.95 | A | A |
| 5.96 | A | A |
| 5.97 | A | A |
| 5.98 | A | A |
| 5.99 | A | A |
| 5.100 | A | A |
| 5.101 | B | C |
| 5.102 | A | A |
| 5.103 | A | A |
| 5.104 | A | A |
| 5.105 | A | A |
| 5.106 | A | B |
| 5.107 | A | A |
| 5.108 | A | A |
| 5.109 | A | A |
| 5.110 | A | A |
| 5.111 | A | A |
| 5.112 | A | A |
| 5.113 | A | A |
| 5.114 | A | A |
| 5.115 | A | A |
| 5.116 | B | C |
| 5.117 | A | B |
| 5.118 | A | B |
| 5.119 | A | A |
| 5.120 | A | A |
| 5.121 | B | B |
| 6.01 | B | C |
| 6.02 | A | A |
| 6.03 | A | A |
| 6.04 | A | A |
| 6.05 | A | C |
| 6.06 | D | D |
| 6.07 | A | A |
| 6.08 | A | A |
| 6.11 | A | A |
| 6.12 | A | A |
| 6.13 | A | C |
| 6.14 | B | C |
| 6.15 | A | A |
| 6.16 | A | A |
| 6.17 | A | A |
| 6.18 | A | A |
| 6.19 | A | B |
| 6.20 | A | A |
| 6.21 | A | A |
| 6.22 | A | A |
| 6.23 | A | A |
| 6.24 | A | B |
| 6.25 | A | A |
| 6.26 | A | A |
| 6.27 | A | A |
| 6.28 | A | B |
| 6.29 | A | A |
| 6.30 | A | A |
| 7.01 | A | B |
| 7.02 | C | C |
| 7.03 | A | B |
| 7.04 | A | B |
| 7.05 | A | A |
| 7.06 | A | B |
| 7.07 | A | B |
| 7.08 | A | A |
| 7.09 | B | C |
| 7.10 | A | A |
| 7.11 | A | B |

### Example 9. Proliferation Assays

The anti-proliferative effect of menin/KMT2A protein:protein interaction inhibitors was assessed in human leukaemia cell lines over the course of three days treatment. Cells were maintained in the appropriate culture medium including 10% FBS at 37° C. with 5%0 CO₂. Cells were seeded in flat bottom 384-well plates at a density of between 1600 and 3200 cells per well in 40 μL of medium, and left overnight at 37° C. Compounds were serially diluted in DMSO using a Tecan (EV0200) liquid handler, then 40 nL was dispensed using an Echo 665 into the cell plate. Cells were left for 72 hours at 37° C., then cell proliferation was assessed using Cell Titre Glo. To do this, the Cell Titre Glo reagent and the cell plate were first equilibrated to room temperature before 30 μL of Cell Titre Glo reagent was added to each well of the cell plate. After adding Cell Titre Glo, the plate was then incubated at room temperature for 30 minutes before luminescence was detected on the Envision using the Ultra Sensitive detection setting.

Percentage inhibition was calculated using the following equation:

$$\% inhibition = 100 * ( HC - Sample ) / ( HC - LC ) , where$$

$$HC = DMSO treated cell and LC = 1 μM staurosporine .$$

Subsequently, the half maximal growth-inhibitory concentration (EC₅₀) was calculated using XLFit (equation 201):

$$Fit = ( A + ( ( B - A ) / ( 1 + ( ( x / C \hat{} D ) ) ) ) , where$$

$$A = bottom , B = top , C = {EC}_{50} and D = slope$$

The results are shown in Table A; key: A: <0.1 μM; B: <1 μM; C: <10 μM; D: >10 μM.

TABLE A
Proliferation assay data

| | MV4-11 | MV4-11 |
|---|---|---|
| Example | WT | M327I |
| 1.011 | B | B |
| 1.013 | B | B |
| 1.029 | A | B |
| 1.031 | A | A |
| 1.032 | A | B |
| 1.036 | A | B |
| 1.039 | A | B |
| 1.043 | A | A |
| 1.044 | B | C |
| 1.051 | A | B |
| 1.063 | A | A |
| 1.066 | A | B |
| 1.068 | A | B |
| 1.070 | A | C |
| 1.077 | B | B |
| 1.080 | A | A |
| 1.084 | C | D |
| 1.089 | B | C |
| 1.092 | A | B |
| 1.104 | A | B |
| 1.106 | A | B |
| 1.107 | B | C |
| 1.113 | A | A |
| 1.148 | A | B |
| 1.149 | B | B |
| 1.151 | A | A |
| 1.153 | A | B |
| 1.154 | A | B |
| 2.01 | A | B |
| 2.02 | A | A |
| 2.03 | B | B |
| 2.05 | A | B |
| 2.06 | B | B |
| 2.07 | A | A |
| 2.09 | A | B |
| 2.10 | A | A |
| 2.11 | A | A |
| 2.12 | A | A |
| 2.13 | A | B |
| 2.14 | A | B |
| 2.15 | A | A |
| 2.16 | A | A |
| 2.18 | A | A |
| 2.19 | A | A |
| 2.20 | A | B |
| 2.21 | A | A |
| 2.23 | A | A |
| 2.24 | A | B |
| 2.25 | B | |
| 2.26 | A | A |
| 2.27 | B | A |
| 2.28 | A | A |
| 2.29 | A | A |
| 2.30 | A | A |
| 2.31 | A | A |
| 2.32 | A | A |
| 2.34 | A | A |
| 2.35 | A | A |
| 2.36 | A | A |
| 3.04 | A | B |
| 3.05 | A | B |
| 3.06 | A | B |
| 3.07 | A | A |
| 3.08 | A | A |
| 3.09 | A | A |
| 3.10 | A | A |
| 3.15 | A | A |
| 3.17 | A | A |
| 3.18 | A | A |
| 3.19 | A | A |
| 3.20 | A | A |
| 3.21 | A | A |
| 3.22 | A | B |
| 3.22 | B | B |
| 3.23 | A | A |
| 3.25 | A | A |
| 3.27 | A | A |
| 3.29 | A | A |
| 3.32 | A | A |
| 3.33 | A | A |
| 3.34 | B | B |
| 3.35 | A | A |
| 3.36 | A | A |
| 3.37 | B | B |
| 3.38 | A | A |
| 3.40 | A | A |
| 3.41 | B | B |
| 3.42 | A | A |
| 3.43 | A | B |
| 3.44 | A | B |
| 3.45 | A | A |
| 3.46 | A | B |
| 3.47 | A | A |
| 3.48 | A | A |
| 3.49 | A | A |
| 3.50 | A | A |
| 3.51 | A | A |
| 3.52 | A | B |
| 3.53 | A | A |
| 3.54 | A | A |
| 3.55 | A | B |
| 3.56 | A | A |
| 3.57 | A | A |
| 3.58 | A | A |
| 3.59 | A | A |
| 3.60 | A | A |
| 3.61 | A | A |
| 3.63 | A | B |
| 3.64 | A | A |
| 3.65 | A | A |
| 3.66 | A | A |
| 3.67 | A | A |
| 3.68 | A | A |
| 3.69 | A | A |
| 3.70 | A | A |
| 3.71 | A | A |
| 3.72 | A | A |
| 3.73 | A | A |
| 3.74 | A | B |
| 3.75 | C | B |
| 3.76 | A | A |
| 3.77 | A | A |
| 3.78 | A | A |
| 3.79 | A | A |
| 3.80 | B | C |
| 3.81 | A | A |
| 3.82 | A | B |
| 3.83 | A | A |
| 3.84 | B | B |
| 3.85 | A | B |
| 3.86 | A | A |
| 3.87 | A | A |
| 3.88 | A | A |
| 4.01 | A | B |
| 4.02 | A | A |
| 4.03 | A | B |
| 4.04 | A | A |
| 5.01 | B | B |
| 5.02 | A | B |
| 5.03 | A | B |
| 5.06 | A | A |
| 5.07 | B | B |
| 5.08 | A | B |
| 5.09 | A | A |
| 5.10 | A | A |
| 5.11 | A | B |
| 5.13 | B | B |
| 5.15 | B | C |
| 5.18 | A | A |
| 5.19 | B | B |
| 5.21 | B | B |
| 5.22 | A | B |
| 5.23 | A | A |
| 5.24 | A | A |
| 5.26 | A | A |
| 5.27 | A | A |
| 5.28 | A | A |
| 5.29 | B | C |
| 5.30 | A | A |
| 5.31 | A | A |
| 5.32 | A | A |
| 5.33 | A | A |
| 5.34 | B | B |
| 5.35 | A | A |
| 5.36 | A | A |
| 5.37 | A | A |
| 5.38 | A | A |
| 5.39 | A | B |
| 5.40 | A | A |
| 5.41 | B | B |
| 5.42 | B | B |
| 5.43 | A | A |
| 5.44 | A | A |
| 5.45 | A | A |
| 5.46 | A | A |
| 5.47 | A | A |
| 5.48 | A | B |
| 5.49 | A | A |
| 5.50 | A | A |
| 5.51 | A | A |
| 5.52 | A | A |
| 5.53 | A | A |
| 5.55 | A | B |
| 5.56 | A | A |
| 5.57 | A | A |
| 5.58 | B | B |
| 5.59 | A | A |
| 5.60 | A | B |
| 5.61 | A | A |
| 5.63 | A | B |
| 5.64 | A | A |
| 5.65 | A | A |
| 5.66 | A | A |
| 5.67 | A | A |
| 5.68 | A | A |
| 5.69 | A | A |
| 5.71 | B | C |
| 5.72 | A | A |
| 5.73 | A | A |
| 5.74 | A | A |
| 5.75 | A | A |
| 5.76 | B | B |
| 5.77 | A | A |
| 5.78 | A | A |
| 5.79 | A | A |
| 5.80 | B | B |
| 5.81 | B | C |
| 5.82 | A | A |
| 5.83 | A | A |
| 5.84 | B | B |
| 5.85 | A | A |
| 5.86 | A | A |
| 5.87 | A | A |
| 5.88 | B | B |
| 5.89 | A | A |
| 5.90 | A | B |
| 5.91 | A | A |
| 5.92 | A | A |
| 5.93 | A | A |
| 5.94 | A | A |
| 5.95 | A | A |
| 5.96 | A | A |
| 5.97 | C | C |
| 5.98 | C | C |
| 5.99 | A | B |
| 5.100 | A | A |
| 5.102 | A | A |
| 5.103 | B | B |
| 5.104 | A | A |
| 5.105 | A | A |
| 5.106 | B | C |
| 5.107 | A | A |
| 5.108 | A | A |
| 5.109 | A | A |
| 5.110 | A | A |
| 5.111 | A | B |
| 5.112 | B | B |
| 5.113 | A | A |
| 5.114 | A | A |
| 5.115 | A | A |
| 5.117 | C | C |
| 5.118 | B | C |
| 5.119 | A | B |
| 5.120 | A | A |
| 6.02 | A | B |
| 6.03 | A | A |
| 6.04 | A | A |
| 6.07 | A | A |
| 6.08 | A | A |
| 6.11 | A | A |
| 6.12 | B | B |
| 6.15 | A | A |
| 6.16 | A | A |
| 6.17 | A | B |
| 6.18 | A | A |
| 6.20 | A | A |
| 6.21 | A | A |
| 6.22 | A | A |
| 6.24 | A | A |
| 6.25 | A | B |
| 6.26 | A | A |
| 6.27 | A | B |
| 6.28 | B | C |
| 6.29 | A | A |
| 6.30 | A | A |
| 7.05 | A | A |
| 7.08 | A | B |
| 7.10 | A | B |
| 7.11 | C | C |

### Example 9. Menin-KMT2A Peptide Inhibition Assays

Proteins: Wild-type and M327I

Compound-mediated disruption of the interaction between menin protein and a KMT2A peptide was measured using a Fluorescence Polarisation assay. Assay buffer (Tris pH 8.0, 50 mM, NaCl₂ 120 mM, CA-630 0.005%, DTT 2 mM) was prepared and, along with test compounds in DMSO, was transferred to a 384-well assay plate. The final DMSO concentration was 1% (v/v). Menin protein (wild-type or M327I mutant) solution was added to a final concentration of 4 nM. The assay plate was incubated for 30 min at 25° C. KMT2A peptide solution was then added to a final concentration of 1 nM and the assay plate was incubated for 60 min at 25° C. Assay plates were read on a BMG plate reader (460-520). The results are shown in Table B; key: A: <0.1 μM; B: <1 μM; C: <10 μM; D: >10 μM.

TABLE B
Fluorescence polarization (FP) data.

| | WT | M327I |
|---|---|---|
| Example | Menin | Menin |
| 1.001 | A | B |
| 1.002 | A | B |
| 1.003 | B | C |
| 1.004 | B | B |
| 1.005 | B | C |
| 1.006 | C | D |
| 1.007 | C | C |
| 1.008 | B | C |
| 1.009 | A | B |
| 1.010 | B | B |
| 1.011 | A | A |
| 1.012 | B | C |
| 1.013 | A | A |
| 1.014 | A | B |
| 1.015 | A | B |
| 1.016 | A | B |
| 1.017 | B | B |
| 1.018 | B | C |
| 1.019 | B | C |
| 1.020 | A | C |
| 1.021 | A | B |
| 1.022 | B | C |
| 1.023 | B | C |
| 1.024 | B | C |
| 1.025 | A | C |
| 1.026 | A | B |
| 1.027 | B | C |
| 1.028 | A | C |
| 1.029 | A | A |
| 1.030 | A | B |
| 1.031 | A | A |
| 1.032 | A | A |
| 1.033 | A | C |
| 1.034 | A | C |
| 1.035 | B | C |
| 1.036 | A | A |
| 1.037 | A | B |
| 1.038 | A | B |
| 1.039 | A | A |
| 1.040 | A | B |
| 1.041 | A | B |
| 1.042 | A | B |
| 1.043 | A | A |
| 1.044 | A | A |
| 1.045 | B | B |
| 1.046 | B | B |
| 1.047 | B | B |
| 1.048 | A | B |
| 1.049 | B | C |
| 1.050 | A | B |
| 1.051 | A | A |
| 1.052 | B | C |
| 1.053 | B | C |
| 1.054 | B | D |
| 1.055 | C | D |
| 1.056 | B | C |
| 1.057 | B | C |
| 1.058 | B | C |
| 1.059 | C | D |
| 1.060 | C | D |
| 1.061 | A | C |
| 1.062 | A | B |
| 1.063 | A | A |
| 1.064 | C | D |
| 1.065 | B | C |
| 1.066 | A | A |
| 1.067 | A | B |
| 1.068 | A | A |
| 1.069 | B | B |
| 1.070 | A | A |
| 1.071 | A | B |
| 1.072 | A | B |
| 1.073 | A | B |
| 1.074 | A | B |
| 1.075 | A | B |
| 1.076 | A | B |
| 1.077 | A | A |
| 1.078 | A | C |
| 1.079 | B | B |
| 1.080 | A | A |
| 1.081 | B | C |
| 1.082 | A | B |
| 1.083 | A | B |
| 1.084 | A | A |
| 1.085 | C | C |
| 1.086 | A | B |
| 1.087 | B | C |
| 1.088 | A | B |
| 1.089 | A | A |
| 1.090 | B | B |
| 1.091 | B | C |
| 1.092 | A | A |
| 1.093 | A | B |
| 1.094 | A | B |
| 1.095 | B | B |
| 1.096 | B | C |
| 1.097 | B | C |
| 1.098 | A | B |
| 1.099 | A | B |
| 1.100 | A | B |
| 1.101 | B | C |
| 1.102 | B | C |
| 1.103 | B | B |
| 1.104 | A | A |
| 1.105 | B | C |
| 1.106 | A | A |
| 1.107 | A | A |
| 1.108 | A | C |
| 1.109 | A | B |
| 1.110 | A | B |
| 1.111 | A | B |
| 1.112 | A | B |
| 1.113 | A | A |
| 1.114 | A | B |
| 1.115 | A | B |
| 1.116 | A | B |
| 1.117 | B | C |
| 1.118 | B | C |
| 1.119 | A | B |
| 1.120 | B | B |
| 1.121 | B | C |
| 1.122 | A | B |
| 1.123 | A | B |
| 1.124 | B | D |
| 1.125 | C | D |
| 1.126 | B | C |
| 1.127 | B | D |
| 1.128 | B | D |
| 1.129 | C | D |
| 1.130 | C | D |
| 1.131 | C | D |
| 1.132 | C | D |
| 1.133 | D | D |
| 1.134 | C | D |
| 1.135 | C | D |
| 1.136 | B | C |
| 1.137 | B | C |
| 1.138 | C | D |
| 1.139 | B | D |
| 1.140 | D | D |
| 1.141 | C | C |
| 1.142 | C | D |
| 1.143 | A | B |
| 1.144 | A | B |
| 1.145 | A | B |
| 1.146 | A | B |
| 1.147 | A | A |
| 1.148 | A | A |
| 1.149 | A | A |
| 2.01 | A | A |
| 2.02 | A | A |
| 2.03 | A | A |
| 2.04 | A | B |
| 2.05 | A | A |
| 2.06 | A | A |
| 2.07 | A | A |
| 2.08 | A | B |
| 2.09 | A | A |
| 2.10 | A | A |
| 2.11 | A | A |
| 2.12 | A | A |
| 2.13 | A | A |
| 2.14 | A | A |
| 2.15 | A | A |
| 2.16 | A | A |
| 2.17 | A | A |
| 2.18 | A | A |
| 2.19 | A | A |
| 2.20 | A | A |
| 2.21 | A | A |
| 2.22 | B | B |
| 2.23 | A | A |
| 2.24 | A | A |
| 2.25 | A | A |
| 2.26 | A | A |
| 3.01 | A | B |
| 3.02 | A | B |
| 3.03 | A | B |
| 3.04 | A | A |
| 3.05 | A | A |
| 3.06 | A | A |
| 3.07 | A | A |
| 3.08 | A | A |
| 3.09 | A | A |
| 3.10 | A | A |
| 3.11 | A | A |
| 3.12 | A | A |
| 3.13 | A | A |
| 3.14 | A | B |
| 3.15 | A | A |
| 3.16 | A | A |
| 3.17 | A | A |
| 3.18 | A | A |
| 3.19 | A | A |
| 3.20 | A | A |
| 3.21 | A | A |
| 3.22 | A | A |
| 3.22 | A | A |
| 3.23 | A | A |
| 3.25 | A | A |
| 3.26 | B | C |
| 3.27 | A | A |
| 3.28 | B | B |
| 3.29 | A | A |
| 3.30 | A | A |
| 3.31 | A | A |
| 3.32 | A | A |
| 3.33 | A | A |
| 3.34 | A | A |
| 3.35 | A | A |
| 3.36 | A | A |
| 3.37 | A | A |
| 3.38 | A | A |
| 4.01 | A | A |
| 4.02 | A | A |
| 4.03 | A | A |
| 5.01 | A | A |
| 5.02 | A | A |
| 5.03 | A | A |
| 5.04 | A | B |
| 5.05 | B | B |
| 5.06 | A | A |
| 5.07 | A | A |
| 5.08 | A | A |
| 5.09 | A | A |
| 5.10 | A | A |
| 5.11 | A | A |
| 5.12 | A | B |
| 5.13 | A | A |
| 5.14 | A | B |
| 5.15 | A | A |
| 5.16 | A | B |
| 5.17 | A | B |
| 5.18 | A | A |
| 5.19 | A | A |
| 5.20 | B | C |
| 5.21 | A | A |
| 5.22 | A | A |
| 5.23 | A | A |
| 5.24 | A | A |
| 5.25 | A | B |
| 5.26 | A | A |
| 5.27 | A | A |
| 5.28 | A | A |
| 5.29 | A | A |
| 6.01 | B | C |
| 6.02 | A | A |
| 6.03 | A | A |
| 6.04 | A | A |
| 6.05 | A | C |
| 6.06 | D | D |
| 6.07 | A | A |
| 6.08 | A | A |
| 6.11 | A | A |
| 6.12 | A | A |
| 6.13 | A | C |
| 7.01 | A | B |
| 7.02 | C | C |
| 7.03 | A | B |
| 7.04 | A | B |

### Example 10. Menin-KMT2A Peptide Inhibition Assay

Proteins: Wild-type, T349M, M327V, M327I, S160T, S160C and G331R

Assay buffer (Tris pH 8.0, 50 mM, NaCl₂ 120 mM, CA-630 0.005%, DTT 2 mM) was prepared and, along with test compounds in DMSO, was transferred to a 384-well assay plate. The final DMSO concentration was 1% (v/v). Menin protein solution was added to a final concentration of 8 nM (wild-type, T349M, M327V, M327I, S160T, S160C) or 12 nM (G331R). The assay plate was spun down for 60 sec at 1000 rpm. KMT2A peptide solution was then added to a final concentration of 4 nM and the assay plate was incubated for 60 min at room temperature. The assay plate was read on a Envision plate reader (Mirror: 681, Excitation: 104, Emission: 208 S channel, vertical, 209 P channel, horizontal).

Data analysis was done by first calculating the % inhibition at each compound concentration following this equation: % inhibition=(Ratio sample−Ratio 0% inhibition)/(Ratio 100% inhibition−Ratio 0% inhibition)*100.

The compound IC₅₀s were calculated using XLfit using the following equations:

$$fit = ( A + ( ( B - A ) / ( 1 + ( ( C / x ) \hat{} D ) ) ) ) , res = ( y - fit ) , where$$

$$A = Bottom , B = Top , C = relative {IC}_{50} and D = Hill Slope .$$

The results are shown in Table C; key: A: <0.1 μM; B: <1 μM; C: <10 μM; D: >10 μM.

TABLE C
Cross-mutant FP assay data

| Exam- | | | | | | | |
|---|---|---|---|---|---|---|---|
| ple | WT | G331R | M327I | M327V | S160C | S160T | T349M |
| 1.013 | A | A | B | B | B | B | B |
| 1.029 | A | A | A | A | B | B | A |
| 1.032 | A | A | B | B | B | B | A |
| 1.043 | A | A | A | A | A | A | A |
| 1.063 | A | A | A | A | A | B | A |
| 1.080 | A | A | A | A | A | A | A |
| 2.10 | A | A | A | A | A | A | A |
| 2.11 | A | A | A | A | A | A | A |
| 2.25 | A | B | B | B | B | B | B |
| 3.07 | A | A | A | A | A | A | A |
| 3.28 | A | A | A | A | A | A | A |
| 3.42 | A | A | A | A | A | A | A |
| 3.49 | A | A | A | A | A | A | A |
| 4.02 | A | A | A | A | A | B | A |
| 5.01 | A | B | B | B | B | B | B |
| 5.02 | A | A | A | A | A | B | A |
| 5.03 | A | A | A | A | A | A | A |
| 5.18 | A | A | A | A | A | A | A |
| 5.19 | A | A | A | A | A | B | A |
| 5.26 | A | A | A | A | A | A | A |
| 5.33 | A | A | A | A | A | A | A |
| 5.56 | A | A | A | A | A | A | A |
| 6.02 | A | A | A | A | B | B | A |
| 6.24 | A | A | A | A | A | A | A |

### Example 11. Subcutaneous (SC) Tumor Model

### Methods:

### Test Agents and Controls

Example 1.063 was formulated in 10% kleptose HPB and 5% Tween 80 made up in water (final pH not less than 5) to the appropriate concentration. Mice were dosed with 10 μL of a compound solution (at the concentrations of 1, 2.5, 5 and 10 mg/mL) per gram of body weight. Doses were adjusted by individual body weight each day. Working stocks of compound were prepared fresh before every treatment. Compound was stored as a solid at room temperature.

### Animals

Female BALB/c nude mice (WT SC and M327I SC) were used when they were approximately 6 to 8 weeks old and weighed between 18-22 g. All animals were quarantined after shipping and allowed to acclimate to recover from shipping-related stress for a minimum of 7 days prior to the study. The general health of the animals was evaluated by a veterinarian and animals with abnormalities were excluded prior to the study. Mice were kept in laminar flow rooms at a constant temperature (23° C.+/−3° C.) and humidity (40%-70%) with 3-5 mice per cage. Autoclaved water and irradiated food was provided ad libitum and animals were maintained on a 12 hour light dark cycle with fluorescent lighting. Cages and bedding were autoclaved before use and both were changed weekly.

### Tumour Model and Cell Culture Method

Human AML MV-4-11 cells (both WT and M327I) were cultured at 37° C. and 5% CO₂ in complete culture media (IMDM supplemented with 10% FBS). Cells in exponential growth were harvested and counted for tumour inoculation. Each mouse was inoculated subcutaneously into the right flank with a single cell suspension of tumour cells. For WT SC, each mouse received 1×10⁷ cells in 100 μL IMDM with Matrigel mixture (1:1 ratio) without serum. For M327I SC, each mouse received 2×10⁷ cells in 200 μL IMDM with Matrigel mixture (1:1 ratio) without serum.

### Study Designs

Example 1.063 was administered orally (PO), twice daily (BID).

The study was initiated at Day 0 with tumour cell inoculation. Mice bearing WT SC tumours were randomly assigned to groups on day 10 post-tumour inoculation according to tumour volume (100-150 mm³) such that the average starting tumour size was the same for each treatment group (n=6-8 mice per group). Treatment with vehicle or Example 1.063 (10, 25, 50 and 100 mg/kg) was initiated on the same day, with twice daily oral dosing for 21 days. Treatment completed on day 31 post inoculation, and the mice were kept in observation to assess tumour burden for 7 more days.

Mice bearing M327I SC tumours were randomly assigned to groups on day 7 post-tumour inoculation according to tumour volume (150-200 mm³) such that the average starting tumour size was the same for each treatment group (n=6 mice per group). Treatment with vehicle or Example 1.063 (10, 25, 50 and 100 mg/kg) was initiated on the same day, with twice daily oral dosing for 28 days. Treatment completed on day 35 post inoculation.

### Animal Monitoring

SC tumour volumes were measured using calipers, on each animal, twice weekly throughout the study period.

### Calculations

Tumour volume (TV) was calculated using the formula:

TV (mm³)=a×b²/2, where “a” and “b” are long and short diameters of a tumour, respectively. Tumour volume data was graphed as the mean tumour volume+/−SEM (mm³).

### Data Analysis

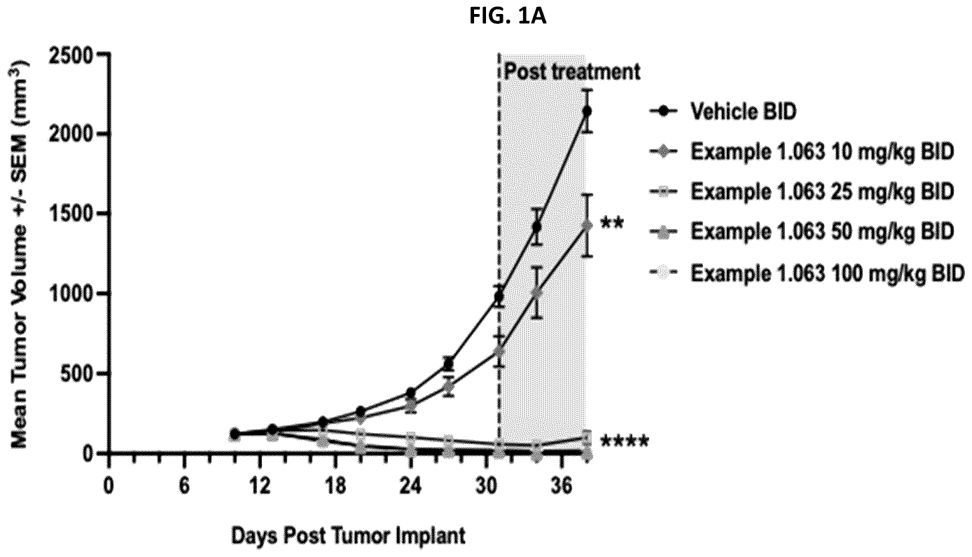

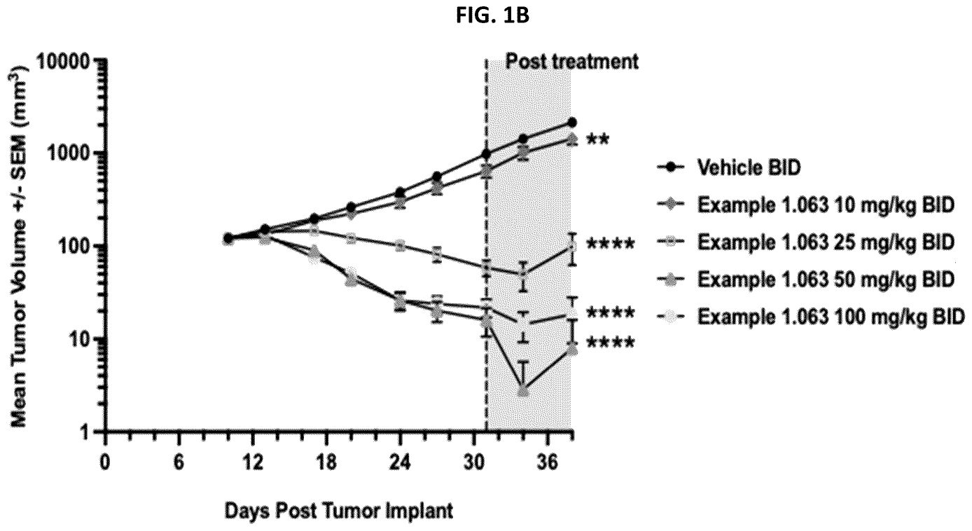

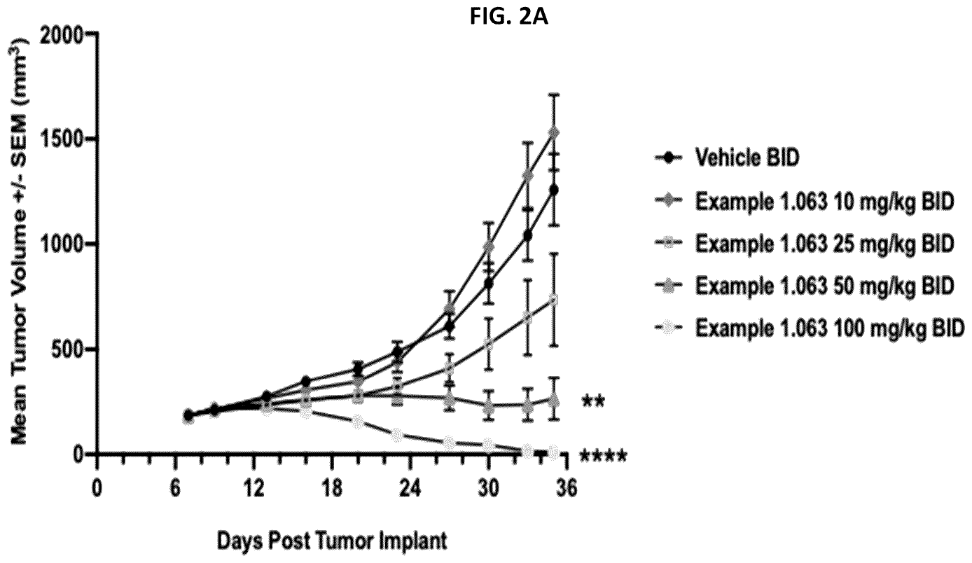

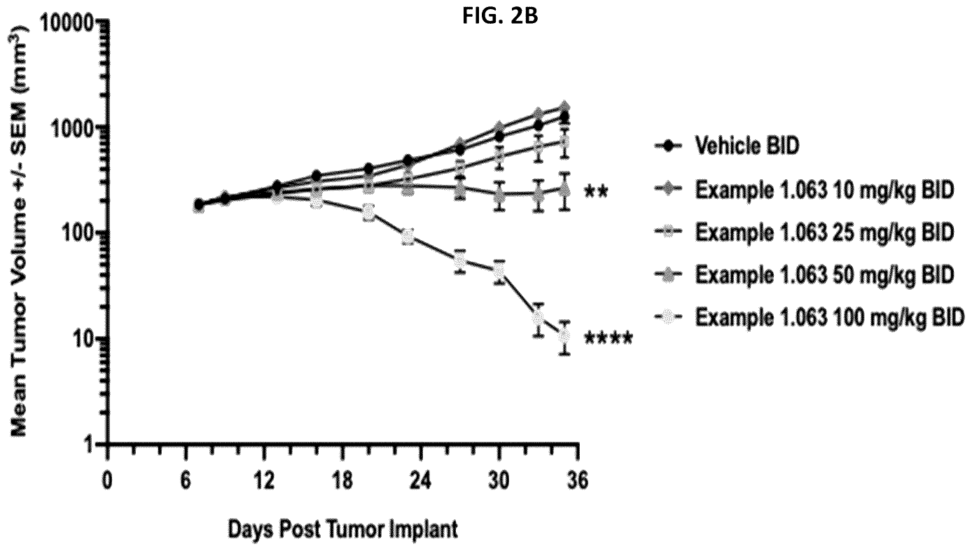

Tumor volume was graphed using Prism Software (Version 10.3.1). Statistical significance was evaluated for Example 1.063 treated groups compared with the vehicle treated controls on the last day of the study, when all mice in all groups remained alive. Differences between groups were considered statistically significant when p≤0.05. Statistical significance for animal tumour volume was calculated using a two-way Anova with Tukey's multiple comparisons testing. The results are shown in FIGS. 1 and 2.

What is claimed is:

1. A compound of Formula (I): 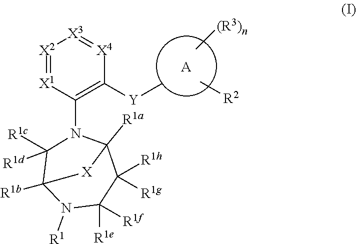
or a pharmaceutically acceptable salt thereof; or an atropisomer thereof; of a pharmaceutically acceptable salt of an atropisomer thereof; wherein:
R¹ is H, a nitrogen protecting group, or 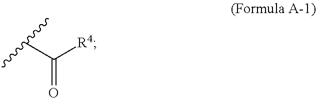
X is CH₂, CH(CH₃), or C(CH₃)₂;
each of R^{1a} and R^{1b} is independently selected from the group consisting of H and CH₃;
each of R^{1c}, R^{1d}, R^{1e}, R^{1f}, R^{1g}, and R^{1h} is independently selected from the group consisting of H, halo, C₁-C₂ alkyl, and C₁-C₂ haloalkyl;
one, two, or three of X¹, X², X³, and X⁴ are N, and the other(s) are an independently selected CR⁵,
Y is O, S, S(O), or SO₂;
Ring A is:
C₆₋₁₀ aryl; or
heteroaryl of 5-10 ring atoms, wherein 1-4 ring atoms are heteroatoms, each independently selected from the group consisting of N, N(H), N(R^{d}), O, and S(O)₀₋₂;
R² is: 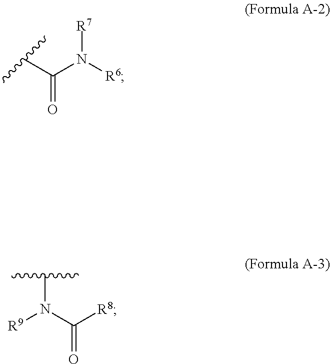
R¹⁰; —C₁-C₄ alkylene-NR^{e}R^{f}; or —OR⁹;
each occurrence of R³ is, independently, selected from the group consisting of: halo; C₁₋₄ alkyl; C₂₋₄ alkynylC₁₋₄ alkoxy; C₁₋₄ haloalkoxy; —S(O)₁₋₂(C₁₋₄ alkyl); and cyano;
n is 0, 1 or 2;
R⁴ is:
C₁₋₈ alkyl or C₂₋₈ alkenyl, each of which is optionally substituted with 1-3 independently selected R^{a},
L₁-C₃₋₁₀ cycloalkyl or L₁-C₃₋₁₀ cycloalkenyl, each of which is optionally substituted with 1-4 substituents independently selected from the group consisting of oxo, R^{b}, and R^{g};
L₁-heterocyclyl or L₁-heterocycloalkenyl of 3-12 ring atoms, wherein 1-3 ring atoms are heteroatoms, each independently selected from the group consisting of N, N(H), N(R^{d}), N(O⁻), O, and S(O)₀₋₂, and wherein the heterocyclyl or heterocycloalkenyl is optionally substituted with 1-4 substituents independently selected from the group consisting of oxo, R^{b}, and R^{g};
L₁-heteroaryl of 5-12 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of oxo, R^{c}, and R^{g}; or
L₁-C₆₋₁₀ aryl optionally substituted with 1-4 substituents independently selected from the group consisting of oxo, R^{c}, and R^{g};
L₁ is a bond or C₁₋₄ alkylene;
R⁵ is H, C₁₋₂ alkoxy or C₁₋₂ alkyl;
each of R⁶ and R⁷ is independently selected from the group consisting of,
H;
C₁₋₆ alkyl optionally substituted with 1-4 independently selected R^{a}; and
C₃₋₈ cycloalkyl optionally substituted with substituents independently selected from the group consisting of oxo, R^{b}, and R^{g}; or
R⁶ and R⁷ together with the nitrogen atom to which each is attached forms a heterocyclyl of 3-8 ring atoms, wherein 1-3 ring atoms are heteroatoms, each independently selected from the group consisting of N, N(H), N(R^{d}), O, and S(O)₀₋₂, and wherein the heterocyclyl is optionally substituted with 1-4 substituents independently selected from the group consisting of oxo, R^{b}, and R^{g};
each of R⁸ and R⁹ is independently selected from the group consisting of,
H;
C₁₋₆ alkyl optionally substituted with 1-4 independently selected R^{a}; and
C₃₋₈ cycloalkyl optionally substituted with substituents independently selected from the group consisting of oxo, R^{b}, and R^{g};
R¹⁰ is:
L₁-heteroaryl of 5-12 ring atoms or heterocyclyl of 5-12 ring atoms, wherein 1-4 ring atoms are heteroatoms, each independently selected from the group consisting of N, N(H), N(R^{d}), O, and S(O)₀₋₂, and wherein each is optionally substituted with 1-4 substituents independently selected from the group consisting of oxo, R^{c}, and R^{g};
L₁-C₆₋₁₀ aryl optionally substituted with 1-4 substituents independently selected from the group consisting of oxo, R^{c}, and R^{g};
C₁₋₆ alkoxy;
C₃₋₈ cycloalkyl optionally substituted with substituents independently selected from the group consisting of oxo, R^{b}, and R^{g}; and
L₁-heterocyclyl or L₁-heterocycloalkenyl of 3-12 ring atoms, wherein 1-3 ring atoms are heteroatoms, each independently selected from the group consisting of N, N(H), N(R^{d}), N(O⁻), O, and S(O)₀₋₂, and wherein the heterocyclyl or heterocycloalkenyl is optionally substituted with 1-4 substituents independently selected from the group consisting of oxo, R^{b}, and R^{g};
each occurrence of R^{a} is independently selected from the group consisting of —OH;-halo; —NR^{e}R^{f}; C₁₋₄ alkoxy; C₁₋₄ haloalkoxy; —C(═O)O(C₁₋₄ alkyl); —C(═O)(C₁₋₄ alkyl); —OC(═O)(C₁₋₄ alkyl); —C(═O)OH; —CONR′R″; —S(O)₁₋₂NR′R″; —S(O)₁₋₂(C₁₋₄ alkyl); and cyano;
each occurrence of R^{b} and R^{c} is independently selected from the group consisting of halo; cyano; C₁₋₁₀ alkyl which is optionally substituted with 1-6 independently selected R^{a}; C₂₋₆ alkenyl; C₂₋₆ alkynyl; C₁₋₄ alkoxy; C₁₋₄ haloalkoxy; —S(O)₁₋₂(C₁₋₄ alkyl); —S(O)(═NH)(C₁₋₄ alkyl); —NR^{e}R^{f}; —OH; —S(O)₁₋₂NR′R″; —C₁₋₄ thioalkoxy; —NO₂; —C(═O)(C₁₋₁₀ alkyl); —C(═O)O(C₁₋₄ alkyl); —OC(═O)(C₁₋₄ alkyl); —C(═O)OH; —C(═O)NR′R″; —NR′C(═O)(C₁₋₄ alkyl) and —SF₅;
each occurrence of R^{d} is independently selected from the group consisting of: C₁₋₆ alkyl optionally substituted with 1-3 independently selected R^{a}; —C(O)(C₁₋₄ alkyl); —C(O)O(C₁₋₄ alkyl); —CONR′R″; —S(O)₁₋₂NR′R″; —S(O)₁₋₂(C₁₋₄ alkyl); —OH; benzyl, and C₁₋₄ alkoxy;
each occurrence of R^{e} and R^{f} is independently selected from the group consisting of: H; C₁₋₆ alkyl optionally substituted with 1-3 substituents each independently selected from the group consisting of NR′R″, —OH, halo, C₁₋₄ alkoxy, and C₁₋₄ haloalkoxy; —C(O)(C₁₋₄ alkyl); —C(O)O(C₁₋₄ alkyl); —CONR′R″; —S(O)₁₋₂NR′R″; —S(O)₁₋₂(C₁₋₄ alkyl); —OH; phenyl; and C₁₋₄ alkoxy;
each occurrence of R⁹ is independently selected from the group consisting of
C₃₋₁₂ cycloalkyl or C₃₋₁₂ cycloalkenyl, each of which is optionally substituted with 1-4 substituents independently selected from the group consisting of oxo, R^{b}, and R^{h};
heterocyclyl or heterocycloalkenyl of 3-12 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo, R^{b}, and R^{h};
heteroaryl of 5-12 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of oxo, R^{e}, and R^{b}; and
C₆₋₁₀ aryl optionally substituted with 1-4 substituents independently selected from the group consisting of oxo, R^{e}, and R^{h};
each occurrence of R^{h} is independently selected from the group consisting of
C₃₋₁₂ cycloalkyl or C₃₋₁₂ cycloalkenyl, each of which is optionally substituted with 1-4 Rⁱ;
heterocyclyl or heterocycloalkenyl of 3-12 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 Rⁱ;
heteroaryl of 5-12 ring atoms, wherein 1-3 ring atoms are heteroatoms, each independently selected from the group consisting of N, N(H), N(R^{d}), O, and S(O)₀₋₂, and wherein the heteroaryl is optionally substituted with 1-4 Rⁱ; and
C₆0.1₀ aryl optionally substituted with 1-4 Rⁱ;
each occurrence of R′ is independently selected from the group consisting of C₁₋₆ alkyl; C₁₋₄ haloalkyl; C₁₋₄ alkoxy; C₁₋₄ haloalkoxy; and halo; and
each occurrence of R′ and R″ is independently selected from the group consisting of H; —OH; and C₁₋₄ alkyl.

2. The compound of claim 1, wherein each of R^{1a} and R^{1b} is H;

3. The compound of claim 1 or 2, wherein X is CH₂.

4. The compound of any one of claims 1-3, wherein each of R^{1c}, R^{1d}, R^{1e}, R^{1f}, R^{1g}, and R^{1h} is H.

5. The compound of any one of claims 1-4, wherein the compound has Formula (I-A):

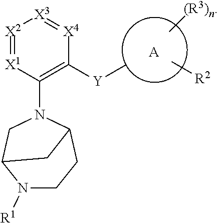

6. The compound of claim 1, wherein the compound has Formula (I-B):

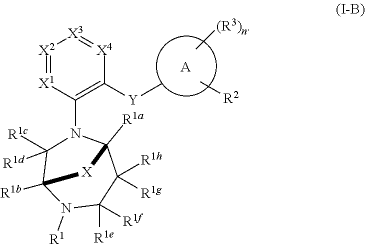

7. The compound of claim 6, wherein each of R^{1a} and R^{1b} is H;

8. The compound of claim 6 or 7, wherein X is CH₂.

9. The compound of any one of claims 6-8, wherein each of R^{1c}, R^{1d}, R^{1e}, R^{1f}, R^{1g}, and R^{1h} is H.

10. The compound of any one of claims 6-9, wherein the compound has Formula (I-A):

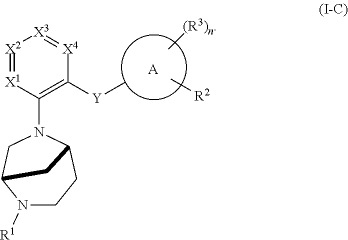

11. The compound of any one of claims 1-10, wherein, wherein R¹ is

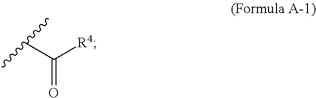

12. The compound of any one of claims 1-11, wherein R⁴ is:
C₁₋₈ alkyl or C₂₋₈ alkenyl, each of which is optionally substituted with 1-3 independently selected R^{a},
L₁-C₃₋₁₀ cycloalkyl or C₃₋₁₀ cycloalkenyl, each of which is optionally substituted with 1-4 substituents independently selected from the group consisting of oxo, R^{b}, and R^{g};
L₁-heterocyclyl or L₁-heterocycloalkenyl of 3-12 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo, R^{b}, and R^{g}; or
L₁-heteroaryl of 5-12 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of oxo, R^{c}, and R^{g}.

13. The compound of any one of claims 1-12, wherein R⁴ is:
C₁₋₈ alkyl, which is optionally substituted with 1-3 independently selected R^{a}, or
L₁-C₃₋₁₀ cycloalkyl, which is optionally substituted with 1-4 substituents independently selected from the group consisting of oxo, R^{b}, and R^{g};
L₁-heterocyclyl of 3-12 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo, R^{b}, and R^{g}; or
L₁-heteroaryl of 5-12 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of oxo, R^{c}, and R^{g}.

14. The compound of any one of claims 1-13, wherein R⁴ in Formula A-1 is L₁-heterocyclyl or L₁-heterocycloalkenyl of 3-12 ring atoms, wherein 1-3 ring atoms are heteroatoms, each independently selected from the group consisting of N, N(H), N(R^{d}), O, and S(O)₀₋₂, and wherein the heterocyclyl or heterocycloalkenyl is optionally substituted with 1-4 substituents independently selected from the group consisting of oxo, R^{b}, and R^{g};

15. The compound of any one of claims 1-14, wherein R⁴ in Formula A-1 is L₁-heterocyclyl of 3-12 ring atoms, wherein 1-3 ring atoms are heteroatoms, each independently selected from the group consisting of N(H), N(R^{d}), O, and S(O)₀₋₂, and wherein the heterocyclyl is optionally substituted with 1-4 substituents independently selected from the group consisting of oxo, R^{b}, and R^{g};

16. The compound of any one of claims 1-15, wherein R⁴ in Formula A-1 is L₁-heterocyclyl of 3-8 ring atoms, wherein 1-2 ring atoms are heteroatoms, each independently selected from the group consisting of N(H), N(R^{d}), O, and S(O)₀₋₂, and wherein the heterocyclyl is optionally substituted with 1-4 substituents independently selected from the group consisting of oxo, R^{b}, and R^{g};

17. The compound of any one of claims 1-15, wherein R⁴ in Formula A-1 is L₁-heterocyclyl of 3-8 ring atoms, wherein 1-2 ring atoms are heteroatoms, each independently selected from the group consisting of N(H), N(R^{d}), and O, and wherein the heterocyclyl is optionally substituted with 1-4 substituents independently selected from the group consisting of oxo, R^{b}, and R^{g};

18. The compound of any one of claims 12-17, wherein the heterocyclyl in Formula A-1 is monocyclic.

19. The compound of claim 18, wherein R⁴ in Formula A-1 is L₁-monocyclic heterocyclyl of 4-6 ring atoms, wherein 1-2 ring atoms are heteroatoms, each independently selected from the group consisting of N(H), N(R^{d}), O, and S(O)₀₋₂, which is optionally substituted with 1-4 substituents independently selected from the group consisting of oxo, R^{b}, and R^{g};

20. The compound of any one of claim 18 or 19, wherein R⁴ in Formula A-1 is L₁-monocyclic heterocyclyl of 4-6 ring atoms, wherein 1-2 ring atoms are heteroatoms, each independently selected from the group consisting of N(H), N(R^{d}), and O, which is optionally substituted with 1-4 substituents independently selected from the group consisting of oxo, R^{b}, and R^{g};

21. The compound of any one of claims 18-20, wherein R⁴ in Formula A-1 is selected from the group consisting of L₁-oxetanyl, L₁-azetidinyl, L₁-thietanyl, L₁-thietanyl-1,1-dioxide, L₁-piperidinyl, L₁-piperazinyl, L₁-pyrrolidinyl, L₁-dioxanyl, L₁-morpholinyl, L₁-tetrahydrofuranyl, L₁-tetrehydrothienyl, and L₁-tetrehydrothienyl-1,1-dioxide, each of which is optionally substituted with 1-4 substituents independently selected from the group consisting of oxo, R^{b}, and R^{g}; optionally wherein R⁴ is L₁-pyrrolidinyl, which is optionally substituted with 1-4 substituents independently selected from the group consisting of oxo, R^{b}, and R^{g}; optionally wherein R⁴ is 2-methylpyrolidin-2-yl.

22. The compound of any one of claims 12-17, wherein the heterocyclyl in Formula A-1 is bicyclic.

23. The compound of claim 22, wherein R⁴ in Formula A-1 is L₁-bicyclic heterocyclyl of 4-12 ring atoms, wherein 1-3 ring atoms are heteroatoms, each independently selected from the group consisting of N, N(H), N(R^{d}), O, and S(O)₀₋₂, and wherein the heterocyclyl is optionally substituted with 1-4 substituents independently selected from the group consisting of oxo, R^{b}, and R^{g}.

24. The compound of claim 22 or 23, wherein R⁴ in Formula A-1 is L₁-bicyclic heterocyclyl of 6-10 ring atoms, wherein 1-3 ring atoms are heteroatoms, each independently selected from the group consisting of N, N(H), N(R^{d}), O, and S(O)₀₋₂, and wherein the heterocyclyl is optionally substituted with 1-4 substituents independently selected from the group consisting of oxo, R^{b}, and R^{g}.

25. The compound of any one of claims 1-14 and 22-24, wherein R⁴ in Formula A-1 is L₁-bicyclic heterocyclyl of 6-8 ring atoms, wherein 1-3 ring atoms are heteroatoms, each independently selected from the group consisting of N, N(H), N(R^{d}), O, and S(O)₀₋₂, and wherein the heterocyclyl is optionally substituted with 1-4 substituents independently selected from the group consisting of oxo, R^{b}, and R^{g}.

26. The compound of any one of claims 1-14 and 22-25, wherein R⁴ in Formula A-1 is selected from the group consisting of L₁-1-azabicyclo[1.1.1]pentanyl, L₁-2-azabicyclo[3.1.0]hexanyl, L₁-3-azabicyclo[3.1.0]hexanyl, L₁-2-azabicyclo[2.1.1]hexanyl, L₁-5-azabicyclo[2.1.1]hexanyl, L₁-3-azabicyclo[3.2.0]heptanyl, L₁-octahydrocyclopenta[c]pyrrolyl, L₁-3-azabicyclo[4.1.0]heptanyl, L₁-7-azabicyclo[2.2.1]heptanyl, L₁-6-azabicyclo[3.1.1]heptanyl, L₁-7-azabicyclo[4.2.0]octanyl, L₁-2-azabicyclo[2.2.2]octanyl, L₁-3-azabicyclo[3.2.1]octanyl, L₁-3-azabicyclo[3.3.0]octanyl, L₁-hexahydro-1H-pyrrolizinyl, L₁-5-azaspiro[2.4]heptanyl, L₁-4-azaspiro[2.4]heptanyl, L₁-azaspiro[2.5]octanyl, L₁-1-azaspiro[3.5]nonanyl, L₁-2-azaspiro[3.5]nonanyl, L₁-7-azaspiro[3.5]nonanyl, L₁-2-azaspiro[4.4]nonanyl, L₁-6-azaspiro[2.6]nonanyl, L₁-1,7-diazaspiro[4.5]decanyl, L₁-7-azaspiro[4.5]decanyl L₁-2,5-diazaspiro[3.6]decanyl, and L₁-3-azaspiro[5.5]undecanyl, each of which is optionally substituted with 1-4 substituents independently selected from the group consisting of oxo, R^{b}, and R^{g}.

27. The compound of any one of claims 1-14 and 22-26, wherein R⁴ in Formula A-1 is selected from the group consisting of L₁-2-azabicyclo[3.1.0]hexanyl, L₁-3-azabicyclo[3.1.0]hexanyl, L₁-2-azabicyclo[2.1.1]hexanyl, L₁-5-azabicyclo[2.1.1]hexanyl, L₁-5-azaspiro[2.4]heptanyl, L₁-3-azabicyclo[3.3.0]octanyl, L₁-3-azabicyclo[3.3.0]octanyl, L₁-hexahydro-1H-pyrrolizinyl, and L₁-4-azaspiro[2.4]heptanyl, each of which is optionally substituted with 1-4 substituents independently selected from the group consisting of oxo, R^{b}, and R^{g}.

28. The compound of any one of claims 22-27, wherein: R⁴ in Formula A-1 is selected from the group consisting of: 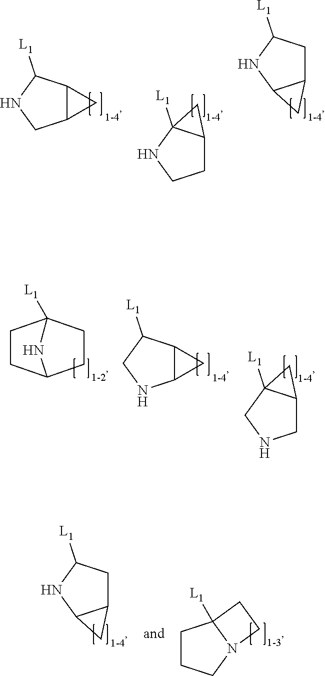
each of which is optionally substituted with 1-4 substituents independently selected from the group consisting of oxo, R^{b}, and R^{g}.

29. The compound of any one of claims 22-28, wherein R⁴ in Formula A-1 is selected from the group consisting of 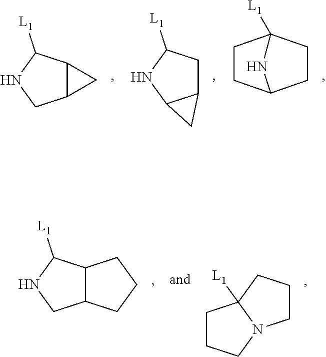
each of which is optionally substituted with 1-4 substituents independently selected from the group consisting of oxo, R^{b}, and R^{g}.

30. The compound of any one of claims 22-28, wherein R⁴ in Formula A-1 has one of the following formulas: 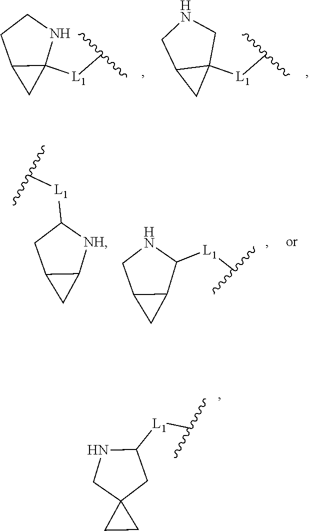
each of which is optionally substituted with 1-4 substituents independently selected from the group consisting of oxo, R^{b}, and R^{g}.

31. The compound of any one of claims 12-30, wherein each occurrence of R^{b} is, independently, selected from the group consisting of: halo; C₁₋₁₀ alkyl which is optionally substituted with 1-6 independently selected R^{a}; C₁₋₄ alkoxy; C₁₋₄ haloalkoxy; —NR^{e}R^{f}; —OH; and —C₁₋₄ thioalkoxy.

32. The compound of any one of claims 12-31, wherein each occurrence of R^{b} is, independently, selected from the group consisting of CH₃, OCH₃, F, CH₂F, and OH.

33. The compound of any one of claims 1-11, wherein R⁴ in Formula A-1 is C₁₋₈ alkyl, which is optionally substituted with 1-3 independently selected R^{a}.

34. The compound of any one of claims 1-11, and 33 wherein R⁴ in Formula A-1 is C₁₋₄ alkyl, which is optionally substituted with 1-2 independently selected R^{a}.

35. The compound of any one of claims 1-11 and 33-34, wherein R⁴ in Formula A-1 is C₁₋₄ alkyl, which is optionally substituted with 1 R^{a}.

36. The compound of any one of claims 1-11 and 33-35, wherein each occurrence of R^{a} is NR^{e}R^{f}; optionally wherein one of R^{e} and R^{f} is H, and the other is C₁-C₃ alkyl.

37. The compound of any one of claims 1-11 and 33-36, wherein each occurrence of R^{a} is NH₂ or NH(CH₃).

38. The compound of any one of claims 1-11 and 33 wherein R⁴ is unsubstituted C₁₋₄ alkyl.

39. The compound of any one of claims 33-38, wherein the substituted or unsubstituted C₁₋₄ alkyl is a straight chain C₁₋₄ alkyl.

40. The compound of any one of claims 23-28, wherein the substituted or unsubstituted C₁₋₄ alkyl is a branched chain C₁₋₄ alkyl.

41. The compound of claim 40, wherein the branched chain C₁₋₄ alkyl comprises —*CH(CH₃)—.

42. The compound of claim 41, wherein the *C has the (R)-configuration.

43. The compound of claim 41, wherein the *C has the (S)-configuration.

44. The compound of any one of claims 1-11, wherein R⁴ in Formula A-1 is L₁-C₃₋₁₀ cycloalkyl, which is optionally substituted with 1-4 substituents independently selected from the group consisting of oxo, R^{b}, and R^{g}.

45. The compound of any one of claims 1-11 and 44, wherein R⁴ in Formula A-1 is L₁-C₃₋₆ cycloalkyl, which is optionally substituted with 1-4 substituents independently selected from the group consisting of oxo, R^{b}, and R^{g}.

46. The compound of any one of claims 1-11 and 44-45, wherein R⁴ in Formula A-1 is L₁-cyclopropyl or L₁-cyclobutyl, each of which is optionally substituted with 1-4 substituents independently selected from the group consisting of oxo, R^{b}, and R^{g}.

47. The compound of any one of claims 1-11, wherein R⁴ in Formula A-1 is L₁-heteroaryl of 5-12 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of oxo, R^{c}, and R^{g}.

48. The compound of any one of claims 1-11 and 47, wherein R⁴ in Formula A-1 is L₁-heteroaryl of 5-6 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of oxo, R^{c}, and R^{g}.

49. The compound of any one of claims 1-11 and 47-48, wherein R⁴ in Formula A-1 is L₁-thienyl, L₁-pyridinyl, L₁-furyl, L₁-oxazolyl, L₁-oxadiazolyl, L₁-pyrrolyl, L₁-imidazolyl, L₁-triazolyl, L₁-thiodiazolyl, L₁-pyrazolyl, L₁-isoxazolyl, L₁-thiadiazolyl, L₁-pyrazinyl, L₁-pyrimidinyl, L₁-pyridazinyl, and L₁-triazinyl.

50. The compound of any one of claims 1-49, wherein L₁ is a bond.

51. The compound of claim 50, wherein a ring carbon atom serves as the point of connection of the cycloalkyl, cycloalkenyl, heterocyclyl, heterocycloalkenyl, heteroaryl, or aryl ring to the C═O in Formula A-1.

52. The compound of claim 50 or 51, wherein R¹ has the formula: 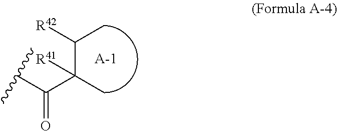
wherein:
R⁴¹ is H or R^{b}; and
R⁴² is H or R^{d}; and
Ring A-1 is an optionally substituted heterocyclyl or an optionally substituted heterocycloalkenyl having 4-8 total ring atoms; optionally having 4-6 total ring atoms, or optionally having five total ring atoms.

53. The compound of claim 52, wherein R⁴¹ is R^{b}.

54. The compound of claim 52 or 53, wherein R⁴¹ is C₁-C₂ alkyl.

55. The compound of any one of claims 52-54, wherein R⁴¹ is CH₃.

56. The compound of any one of claims 52-54, wherein R⁴¹ is H.

57. The compound of any one of claims 52-55, wherein R¹ is:

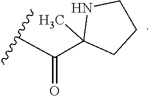 [SMILES: *C(=O)C1(C)CCCN1]

58. The compound of claim 57, wherein the carbon atom attached to the CH₃ has the (R)-configuration.

59. The compound of claim 57, wherein the carbon atom attached to the CH₃ has the (S)-configuration.

60. The compound of any one of claims 1-49, wherein L₁ is C₁₋₄ alkylene.

61. The compound of claim 60, wherein a ring carbon atom serves as the point of connection of the cycloalkyl, cycloalkenyl, heterocyclyl, heterocycloalkenyl, heteroaryl, or aryl ring to L₁.

62. The compound of claim 60 or 61, wherein the C₁₋₄ alkylene is a straight chain alkylene.

63. The compound of any one of claims 60-62, wherein C₁₋₄ alkylene is a branched chain alkylene.

64. The compound of claim 63, wherein the branched chain alkylene comprises CH(CH₃)—.

65. The compound of claim 64, wherein the *C has the (R)-configuration.

66. The compound of claim 64, wherein the *C has the (S)-configuration.

67. The compound of any one of claims 1-66, wherein R² is:

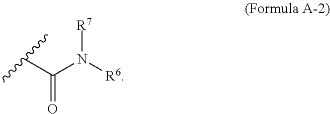

68. The compound of claim 67, wherein each of R⁶ and R⁷ is an independently selected C₁₋₄ alkyl.

69. The compound of claim 67 or 68, wherein one of R⁶ and R⁷ is —CH₂CH₃, and the other is —CH(CH₃)₂.

70. The compound of any one of claims 1-66, wherein R² is:

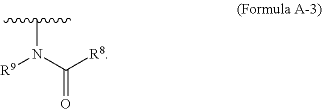

71. The compound of claim 70, wherein each of R⁸ and R⁹ is an independently selected C₁₋₄ alkyl.

72. The compound of claim 70 or 71, wherein one of R⁶ and R⁷ is —CH₂CH₃, and the other is —CH(CH₃)₂.

73. The compound of any one of claims 1-66, wherein R² is R¹⁰.

74. The compound of claim 73, wherein R¹⁰ is L₁-heteroaryl of 5-12 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of oxo, R^{c}, and R^{g}.

75. The compound of claim 73 or 74, wherein R¹⁰ is L₁-heteroaryl of 5-6 ring atoms, wherein 1-4 ring atoms are heteroatoms, each independently selected from the group consisting of N, N(H), N(R^{d}), O, and S(O)₀₋₂, and wherein the heteroaryl is optionally substituted with 1-4 substituents independently selected from the group consisting of oxo, R^{c}, and R^{g}.

76. The compound of any one of claims 63-65, wherein R¹⁰ is selected from the group consisting of L₁-thienyl, L₁-pyridinyl, L₁-furyl, L₁-oxazolyl, L₁-oxadiazolyl, L₁-pyrrolyl, L₁-imidazolyl, L₁-triazolyl, L₁-thiodiazolyl, L₁-pyrazolyl, L₁-isoxazolyl, L₁-thiadiazolyl, L₁-pyrazinyl, L₁-pyrimidinyl, L₁-pyridazinyl, and L₁-triazinyl, each of which is optionally substituted with 1-4 substituents independently selected from the group consisting of oxo, R^{c}, and R^{g}.

77. The compound of any one of claims 74-76, wherein R¹⁰ is selected from the group consisting of

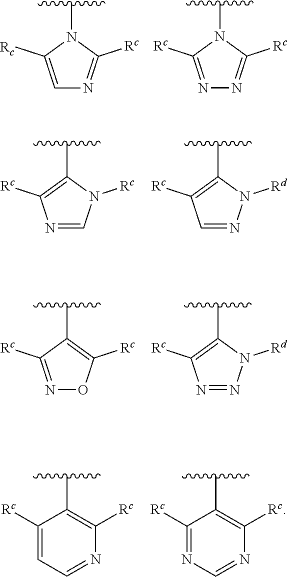

78. The compound of claim 76, wherein each R^{c} is, independently, C₁₋₄ alkyl, C₁₋₄ haloalkyl, or C₃₋₆ cycloalkyl.

79. The compound of claim 76, wherein each R^{d} is, independently, C₁₋₄ alkyl or C₃₋₆ cycloalkyl.

80. The compound of any one of claims 74-79, wherein R¹⁰ is selected from the group consisting of 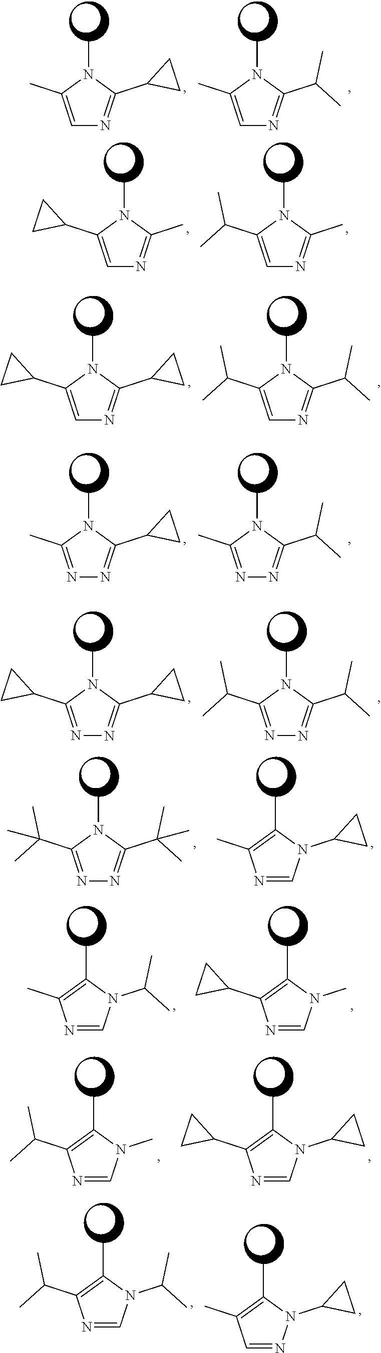 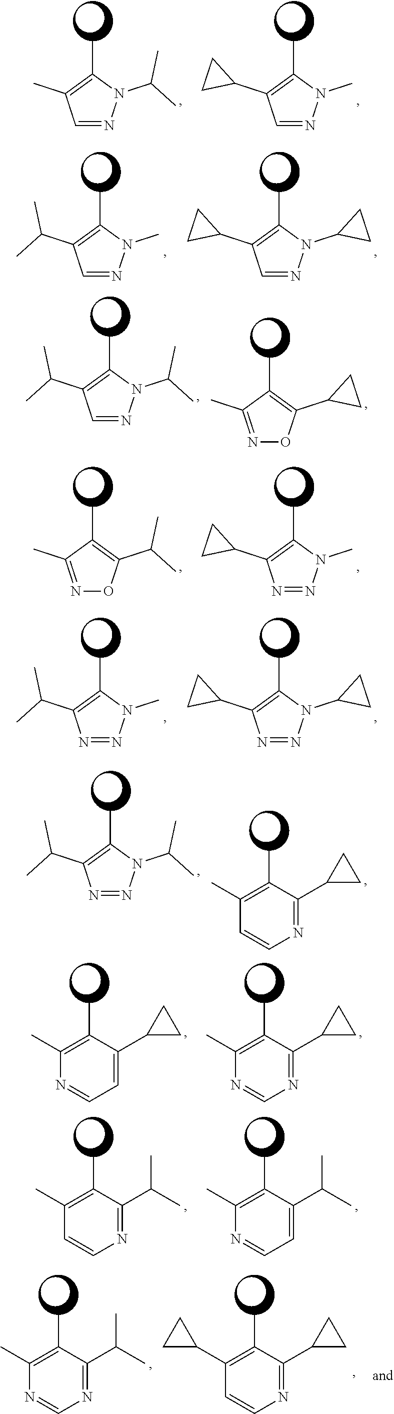 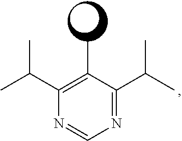
wherein the shaded circle represents the point of attachment to ring A.

81. The compound of any one of claims 1-80, wherein two or three of X¹, X², X³, and X⁴ are N, and the others are an independently selected CR⁵.

82. The compound of any one of claims 1-81, wherein two of X¹, X², X³, and X⁴ are N, and the others are an independently selected CR⁵.

83. The compound of any one of claims 1-82, wherein X¹ and X³ are N, and X² and X⁴ are an independently selected CR⁵.

84. The compound of claim 83, wherein each occurrence of R⁵ is H.

85. The compound of any one of claims 1-81, wherein three of X¹, X², X³, and X⁴ are N and the other is CR⁵.

86. The compound of any one of claims 1-81, wherein X¹, X³, and X⁴ are N and X² is CR⁵.

87. The compound of claim 86, wherein R⁵ is H.

88. The compound of any one of claims 1-87, wherein ring A is phenyl.

89. The compound of any one of claims 1-88, wherein ring A has the

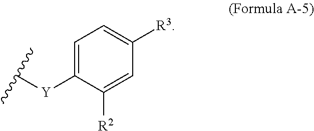

90. The compound of any one of claims 1-88, wherein n is 1.

91. The compound of any one of claims 1-90, wherein R³ is halo.

92. The compound of any one of claims 1-91, wherein R³ is fluoro.

93. The compound of any one of claims 1-92, wherein Y is O.

94. The compound of claim 1, wherein the compound has the formula: 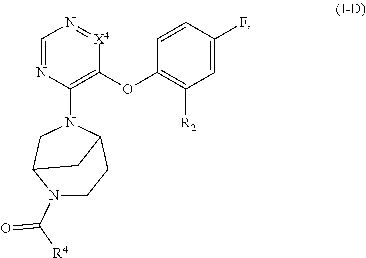
optionally wherein X⁴ is CH.

95. The compound of claim 1, wherein the compound has the formula: 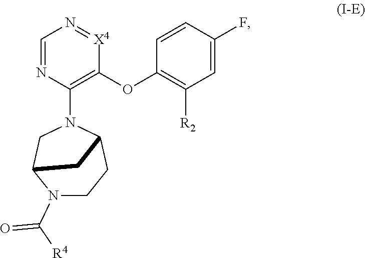
optionally wherein X⁴ is CH.

96. The compound of any one of claims 1-95, wherein the compound is a compound selected from the group consisting of the compounds exemplified in Examples 1.10-5.29, or a pharmaceutically acceptable salt thereof, or an atropisomer thereof, of a pharmaceutically acceptable salt of an atropisomer thereof.

97. A pharmaceutical composition comprising a compound of Formula (I), or a pharmaceutically acceptable salt thereof; or an atropisomer thereof; of a pharmaceutically acceptable salt of an atropisomer thereof as claimed in any one of claims 1-96; and a pharmaceutically acceptable carrier.

98. A method of inhibiting menin-MLL-interaction in a sample, the method comprising contacting the sample with a compound, or a pharmaceutically acceptable salt thereof, or an atropisomer thereof, or a pharmaceutically acceptable salt of an atropisomer thereof, as claimed in any one of claims 1-96.

99. A method for inhibiting menin-MLL interaction in a patient in need thereof, the method comprising administering to the patient a therapeutically effective amount of a compound of Formula (I), or a pharmaceutically acceptable salt thereof, or an atropisomer thereof, or a pharmaceutically acceptable salt of an atropisomer thereof, as claimed in any one of claims 1-96.

100. The method of claim 99, wherein the patient has cancer.

101. The method of claim 100, wherein the cancer is selected from the group consisting of acute lymphoblastic leukaemia, acute myeloid leukaemia, childhood medulloblastoma, chronic lymphocytic leukaemia, diffuse large B cell lymphoma, follicular lymphoma, glioblastoma, liver cancer, myelodysplastic syndrome, pancreatic cancer, prostate cancer, renal cell carcinoma, and triple negative breast cancer.

102. The method of claim 99, wherein the patient has diabetes.

103. The method of claim 102, wherein the diabetes is type 1 diabetes or type 2 diabetes.

104. A method of treating cancer in a patient in need of such treatment, the method comprising administering a compound of Formula (I), or a pharmaceutically acceptable salt thereof; or an atropisomer thereof; of a pharmaceutically acceptable salt of an atropisomer thereof, as claimed in any one of claims 1-96.

105. A method of treating diabetes in a patient in need of such treatment, the method comprising administering a compound of Formula (I), or a pharmaceutically acceptable salt thereof; or an atropisomer thereof; of a pharmaceutically acceptable salt of an atropisomer thereof, as claimed in any one of claims 1-96.
